(19) United States
(12) Patent Application Publication
(10) Pub. No.: US 2023/0165970 A1
(43) Pub. Date: Jun. 1, 2023

(54) COMPOSITIONS AND METHODS FOR THE TREATMENT OF BACTERIAL INFECTIONS

(71) Applicant: Cidara Therapeutics, Inc., San Diego, CA (US)

(72) Inventors: Suzanne AKERS-RODRIGUEZ, La Mesa, CA (US); James M. BALKOVEC, Martinsville, NJ (US); Daniel C. BENSEN, Carlsbad, CA (US); Timothy BLIZZARD, Princeton, NJ (US); Allen BORCHARDT, San Diego, CA (US); Thomas P. BRADY, San Diego, CA (US); Zhi-Yong CHEN, La Jolla, CA (US); Quyen-Quyen Thuy DO, San Diego, CA (US); Simon DOEHRMANN, San Diego, CA (US); Joanne M. FORTIER, San Diego, CA (US); Wanlong JIANG, San Diego, CA (US); Thanh LAM, San Diego, CA (US); James LEVIN, San Marcos, CA (US); Alain NONCOVICH, La Jolla, CA (US); Leslie W. TARI, Solana Beach, CA (US)

(21) Appl. No.: 16/956,011

(22) PCT Filed: Dec. 19, 2018
(86) PCT No.: PCT/US2018/066546

§ 371 Date: Jun. 19, 2020

Related U.S. Application Data
(60) Provisional application No. 62/725,083, filed on Aug. 30, 2018.
(60) Provisional application No. 62/696,758, filed on Jul. 11, 2018.
(60) Provisional application No. 62/608,510, filed on Dec. 20, 2017.

(51) Int. Cl. A61K 47/68; A61P 31/04

(52) U.S. Cl. CPC A61K 47/6811; A61K 47/6889; A61P 31/04

(57) ABSTRACT

Compositions and methods for the treatment of bacterial infections include conjugates containing an Fc domain covalently linked to one or more monomers of cyclic heptapeptides or one or more dimers of cyclic heptapeptides. In particular, conjugates can be used in the treatment of bacterial infections caused by Gram-negative bacteria.

### SEQUENCE LISTING

The instant application contains a Sequence Listing which has been submitted electronically in ASCII format and is hereby incorporated by reference in its entirety. Said ASCII copy, created on Apr. 19, 2021 is named 50945-035004_Sequence_Listing_4.19.21_ST25 and is 82,452 bytes in size.

### BACKGROUND

The need for novel antibacterial treatments for bacterial infections is significant and especially critical in the medical field. Antibacterial resistance is a serious global healthcare threat. Polymyxins are a class of antibiotics that exhibit potent antibacterial activities against Gram-negative bacteria. However, the use of polymyxins as an antibiotic has been limited due to the associated toxicity and adverse effects (e.g., nephrotoxicity). Moreover, mcr-1, a plasmid-borne gene conferring bacterial resistance to polymyxins, has a high potential for dissemination and further threatens the efficacy of this class of antibiotics.

Because of the shortcomings of existing antibacterial treatments, combined with the emergence of multidrug-resistant Gram-negative bacteria, there is a need in the art for improved antibacterial therapies having greater efficacy, bioavailability, and reduced toxicity.

### SUMMARY

The disclosure relates to conjugates, compositions, and methods for inhibiting bacterial growth (e.g., Gram-negative bacterial growth) and for the treatment of bacterial infections (e.g., Gram-negative bacterial infections). In particular, such conjugates contain monomers or dimers of cyclic heptapeptides conjugated to Fc domains. The monomers or dimers of cyclic heptapeptides in the conjugates bind to lipopolysaccharides (LPS) in the cell membrane of Gram-negative bacteria to disrupt and permeabilize the cell membrane, leading to cell death and/or sensitization of the Gram-negative bacteria to other antibiotics, and the Fc domains in the conjugates bind to FcγRs (e.g., FcRn, FcγRI, FcγRIIa, FcγRIIc, FcγRIIIa, and FcγRIIIb) on immune cells, e.g., neutrophils, to activate phagocytosis and effector functions, such as antibody-dependent cell-mediated cytotoxicity (ADCC), thus leading to the engulfment and destruction of bacterial cells by immune cells and further enhancing the antibacterial activity of the conjugates.

In one aspect, the disclosure features a conjugate described by formula (1):

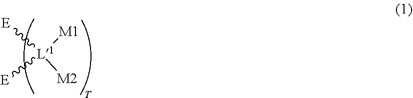

wherein each M1 includes a first cyclic heptapeptide including a linking nitrogen and each M2 includes a second cyclic heptapeptide including a linking nitrogen; each E includes an Fc domain monomer (e.g., an Fc domain monomer having the sequence of any one of SEQ ID NOs: 1-14, 1-29, or 1-31); L′¹ in each M2-L′¹-M1 is a linker covalently attached to a sulfur atom of a hinge cysteine in each E and to the linking nitrogen in each of M1 and M2; T is an integer from 1, 2, 3, 4, or 5, the two squiggly lines connected to the two Es indicate that each M2-L′¹-M1 is covalently attached to a pair of sulfur atoms of two hinge cysteines in the two Es, or a pharmaceutically acceptable salt thereof. When T is greater than 1 (e.g., T is 2, 3, 4, or 5), each M2-L′¹-M1 may be independently selected (e.g., independently selected from any of the M2-L′¹-M1 structures described herein).

In another aspect, the invention features a conjugate described by formula (2):

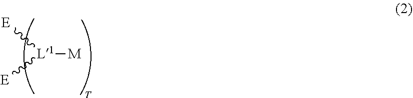

wherein each M includes a cyclic heptapeptide including a linking nitrogen; each E includes an Fc domain monomer (e.g., an Fc domain monomer having the sequence of any one of SEQ ID NOs: 1-14, 1-29, or 1-31); L′¹ in each L′¹-M is a linker covalently attached to a sulfur atom in a hinge cysteine in E and to the linking nitrogen in M; T is 1, 2, 3, 4, or 5, the two squiggly lines connected to the two Es indicate that each L′¹-M is covalently attached to a pair of sulfur atoms of two hinge cysteines in the two Es, or a pharmaceutically acceptable salt thereof.

In some embodiments, each E includes an Fc domain monomer having the sequence of any one of SEQ ID Nos: 1-14, 1-29, or 1-31.

In some embodiments of these aspects, each E comprises the sequence

(SEQ ID NO: 10)
MVRSDKTHTCPPCPPC*KC*PAPELLGGPSVFLFPPKPKD
TLMISRTPEVTCVVVDVSHEDPEVKFNWYVDGVEVHNAKTK
PREEQYNSTYRVVSVLTVLHQDWLNGKEYKCKVSNKALPA
PIEKTISKAKGQPREPQVYTLPPSREEMTKNQVSLTCLVK
GFYPSDIAVEWESNGQPENNYKTTPPVLDSDGSFFLYSKL
TVDKSRWQQGNVFSCSVMHEALHNHYTQKSLSLSPGKHHH
HHH.

In some embodiments, at least one of the pair of sulfur atoms is the sulfur atom corresponding to (e.g., the sulfur atom of) a hinge cysteine of SEQ ID NO: 10, i.e., Cys10, Cys13, Cys16, or Cys18 of SEQ ID NO: 10. In some embodiments, the pair of sulfur atoms are the sulfur atoms corresponding to (e.g., the sulfur atoms of) Cys10 and Cys13 in SEQ ID NO: 10, Cys10 and Cys16 in SEQ ID NO: 10, Cys 30 and Cys18 in SEQ ID NO: 10, Cys13 and Cys 38 in SEQ ID NO: 10, Cys13 and Cys 38 in SEQ ID NO: 10, and/or Cys 36 and Cys 38 in SEQ ID NO: 10.

In some embodiments, when T is 2, the pair of sulfur atoms are (e.g., the sulfur atoms corresponding to) Cys10 and Cys13 in SEQ ID NO: 10 or Cys 36 and Cys 38 in SEQ ID NO: 10.

In some embodiments, the pair of sulfur atoms include one sulfur atom of a cysteine from each E, i.e., L′¹-M along with the sulfur atoms to which it is attached forms a bridge between two Fc domains (e.g., two Fc domains comprising the sequence of SEQ ID NO: 10). In some embodiments, the pair of sulfur atoms are the sulfur atom corresponding to (e.g., the sulfur atom of) Cys10 of SEQ ID NO: 10 from one E and the sulfur atom corresponding to (e.g., the sulfur atom of) Cys10 of SEQ ID NO: 10 from another E. In some embodiments, the pair of sulfur atoms are the sulfur atom corresponding to (e.g., the sulfur atom of) Cys13 of SEQ ID NO: 10 from one E and the sulfur atom corresponding to (e.g., the sulfur atom of) Cys13 of SEQ ID NO: 10 from another E. In some embodiments, the pair of sulfur atoms are the sulfur atom corresponding to (e.g., the sulfur atom of) Cys16 of SEQ ID NO: 10 from one E and the sulfur atom corresponding to (e.g., the sulfur atom of) Cys16 of SEQ ID NO: 10 from another E. In some embodiments, the pair of sulfur atoms are the sulfur atom corresponding to (e.g., the sulfur atom of) Cys18 of SEQ ID NO: 10 from one E and the sulfur atom corresponding to (e.g., the sulfur atom of) Cys18 of SEQ ID NO: 10 from another E.

In some embodiments, when T is 2, the pairs of sulfur atoms are the sulfur atom corresponding to (e.g., the sulfur atom of) Cys10 of SEQ ID NO: 10 from one E and the sulfur atom corresponding to (e.g., the sulfur atom of) Cys10 of SEQ ID NO: 10 from another E and the sulfur atom corresponding to (e.g., the sulfur atom of) Cys13 of SEQ ID NO: 10 from one E and the sulfur atom corresponding to (e.g., the sulfur atom of) Cys13 of SEQ ID NO: 10 from another E. In some embodiments, when T is 2, the pairs of sulfur atoms are the sulfur atom corresponding to (e.g., the sulfur atom of) Cys10 of SEQ ID NO: 10 from one E and the sulfur atom corresponding to (e.g., the sulfur atom of) Cys10 of SEQ ID NO: 10 from another E and the sulfur atom corresponding to (e.g., the sulfur atom of) Cys16 of SEQ ID NO: 10 from one E and the sulfur atom corresponding to (e.g., the sulfur atom of) Cys16 of SEQ ID NO: 10 from another E. In some embodiments, when T is 2, the pairs of sulfur atoms are the sulfur atom corresponding to (e.g., the sulfur atom of) Cys10 of SEQ ID NO: 10 from one E and the sulfur atom corresponding to (e.g., the sulfur atom of) Cys10 of SEQ ID NO: 10 from another E and the sulfur atom corresponding to (e.g., the sulfur atom of) Cys18 of SEQ ID NO: 10 from one E and the sulfur atom corresponding to (e.g., the sulfur atom of) Cys18 of SEQ ID NO: 10 from another E.

In some embodiments, when T is 2, the pairs of sulfur atoms are the sulfur atom corresponding to (e.g., the sulfur atom of) Cys13 of SEQ ID NO: 10 from one E and the sulfur atom corresponding to (e.g., the sulfur atom of) Cys13 of SEQ ID NO: 10 from another E and the sulfur atom corresponding to (e.g., the sulfur atom of) Cys16 of SEQ ID NO: 10 from one E and the sulfur atom corresponding to (e.g., the sulfur atom of) Cys16 of SEQ ID NO: 10 from another E. In some embodiments, when T is 2, the pairs of sulfur atoms are the sulfur atom corresponding to (e.g., the sulfur atom of) Cys13 of SEQ ID NO: 10 from one E and the sulfur atom corresponding to (e.g., the sulfur atom of) Cys13 of SEQ ID NO: 10 from another E and the sulfur atom corresponding to (e.g., the sulfur atom of) Cys18 of SEQ ID NO: 10 from one E and the sulfur atom corresponding to (e.g., the sulfur atom of) Cys18 of SEQ ID NO: 10 from another E.

In some embodiments, when T is 2, the pairs of sulfur atoms are the sulfur atom corresponding to (e.g., the sulfur atom of) Cys16 of SEQ ID NO: 10 from one E and the sulfur atom corresponding to (e.g., the sulfur atom of) Cys16 of SEQ ID NO: 10 from another E and the sulfur atom corresponding to (e.g., the sulfur atom of) Cys18 of SEQ ID NO: 10 from one E and the sulfur atom corresponding to (e.g., the sulfur atom of) Cys18 of SEQ ID NO: 10 from another E.

In some embodiments, when T is 3, the pairs of sulfur atoms are the sulfur atom corresponding to (e.g., the sulfur atom of) Cys10 of SEQ ID NO: 10 from one E and the sulfur atom corresponding to (e.g., the sulfur atom of) Cys10 of SEQ ID NO: 10 from another E; the sulfur atom corresponding to (e.g., the sulfur atom of) Cys13 of SEQ ID NO: 10 from one E and the sulfur atom corresponding to (e.g., the sulfur atom of) Cys13 of SEQ ID NO: 10 from another E; and the sulfur atom corresponding to (e.g., the sulfur atom of) Cys16 of SEQ ID NO: 10 from one E and the sulfur atom corresponding to (e.g., the sulfur atom of) Cys16 of SEQ ID NO: 10 from another E. In some embodiments, when T is 3, the pairs of sulfur atoms are the sulfur atom corresponding to (e.g., the sulfur atom of) Cys10 of SEQ ID NO: 10 from one E and the sulfur atom corresponding to (e.g., the sulfur atom of) Cys10 of SEQ ID NO: 10 from another E; the sulfur atom corresponding to (e.g., the sulfur atom of) Cys13 of SEQ ID NO: 10 from one E and the sulfur atom corresponding to (e.g., the sulfur atom of) Cys13 of SEQ ID NO: 10 from another E; and the sulfur atom corresponding to (e.g., the sulfur atom of) Cys18 of SEQ ID NO: 10 from one E and the sulfur atom corresponding to (e.g., the sulfur atom of) Cys18 of SEQ ID NO: 10 from another E. In some embodiments, when T is 3, the pairs of sulfur atoms are the sulfur atom corresponding to (e.g., the sulfur atom of) Cys10 of SEQ ID NO: 10 from one E and the sulfur atom corresponding to (e.g., the sulfur atom of) Cys10 of SEQ ID NO: 10 from another E; the sulfur atom corresponding to (e.g., the sulfur atom of) Cys18 of SEQ ID NO: 10 from one E and the sulfur atom corresponding to (e.g., the sulfur atom of) Cys18 of SEQ ID NO: 10 from another E; and the sulfur atom corresponding to (e.g., the sulfur atom of) Cys16 of SEQ ID NO: 10 from one E and the sulfur atom corresponding to (e.g., the sulfur atom of) Cys16 of SEQ ID NO: 10 from another E. In some embodiments, when T is 3, the pairs of sulfur atoms are the sulfur atom corresponding to (e.g., the sulfur atom of) Cys13 of SEQ ID NO: 10 from one E and the sulfur atom corresponding to (e.g., the sulfur atom of) Cys13 of SEQ ID NO: 10 from another E; the sulfur atom corresponding to (e.g., the sulfur atom of) Cys18 of SEQ ID NO: 10 from one E and the sulfur atom corresponding to (e.g., the sulfur atom of) Cys18 of SEQ ID NO: 10 from another E; and the sulfur atom corresponding to (e.g., the sulfur atom of) Cys16 of SEQ ID NO: 10 from one E and the sulfur atom corresponding to (e.g., the sulfur atom of) Cys16 of SEQ ID NO: 10 from another E.

In some embodiments, when T is 3, the pairs of sulfur atoms are the sulfur atom corresponding to (e.g., the sulfur atom of) Cys10 of SEQ ID NO: 10 from one E and the sulfur atom corresponding to (e.g., the sulfur atom of) Cys10 of SEQ ID NO: 10 from another E; the sulfur atom corresponding to (e.g., the sulfur atom of) Cys13 of SEQ ID NO: 10 from one E and the sulfur atom corresponding to (e.g., the sulfur atom of) Cys13 of SEQ ID NO: 10 from another E; the sulfur atom corresponding to (e.g., the sulfur atom of) Cys16 of SEQ ID NO: 10 from one E and the sulfur atom corresponding to (e.g., the sulfur atom of) Cys16 of SEQ ID NO: 10 from another E; and the sulfur atom corresponding to (e.g., the sulfur atom of) Cys18 of SEQ ID NO: 10 from one E and the sulfur atom corresponding to (e.g., the sulfur atom of) Cys18 of SEQ ID NO: 10 from another E.

In some embodiments, the conjugate has the structure:

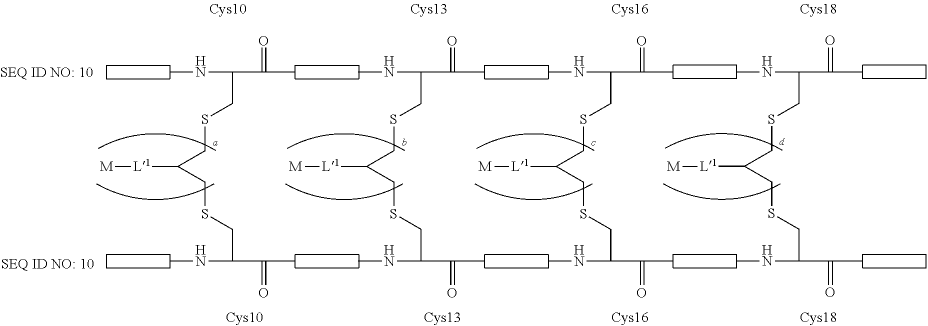

wherein each of a, b, c, and d is, independently, 0 or 1 and wherein when a, b, c, or d is 0, the two sulfur atoms form a disulfide bond.

In some embodiments, a is 1 and b, c, and d are 0. In some embodiments, a and b are 1 and c and d are 0. In some embodiments, a and c are 1 and b and d are 0. In some embodiments, a and d are 1 and b and c are 0. In some embodiments, a, b, and c are 1 and d is 0. In some embodiments, a, b, and d are 1 and c is 0. In some embodiments, a, c, and d are 1 and b is 0. In some embodiments, b and c are 1 and a and d are 0. In some embodiments, b and d are 1 and a and c are 0. In some embodiments, b, c, and d are 1 and a is 0. In some embodiments, c and d are 1 and a and b are 0. In some embodiments, a, b, c, and d are 1.

In some embodiments of these aspects, each E comprises the sequence

(SEQ ID NO: 4)
MVRSDKTHTCPPCPAPELLGGPSVFLFPPKPKDTLMISRT
PEVTCVVDVSHEDPEVKFNWYVDGVEVHNAKTKPREEQYN
STYRVVSVLTVLHQDWLNGKEYKCKVSNKALPAPIEKTIS
KAKGQPREPQVYTLPPSREEMTKNQVSLTCLVKGFYPSDI
AVEWESNGQPENNYKTTPPVLDSDGSFFLYSKLTVDKSRW
QQGNVFSCSVMHEALHNHYTQKSLSLSPGK.

In some embodiments, at least one of the pair of sulfur atoms is the sulfur atom corresponding to (e.g., the sulfur atom of) a hinge cysteine of SEQ ID NO: 4, i.e., Cys10 and/or Cys13. In some embodiments, the pair of sulfur atoms are the sulfur atoms corresponding to (e.g., the sulfur atoms of) Cys10 and Cys13 in SEQ ID NO: 4.

In some embodiments, the pair of sulfur atoms include one sulfur atom of a cysteine from each E, i.e., L′¹-M along with the sulfur atoms to which it is attached forms a bridge between two Fc domains (e.g., two Fc domains comprising the sequence of SEQ ID NO: 4). In some embodiments, the pair of sulfur atoms are the sulfur atom corresponding to (e.g., the sulfur atom of) Cys10 of SEQ ID NO: 4 from one E and the sulfur atom corresponding to (e.g., the sulfur atom of) Cys10 of SEQ ID NO: 4 from another E. In some embodiments, the pair of sulfur atoms are the sulfur atom corresponding to (e.g., the sulfur atom of) Cys13 of SEQ ID NO: 4 from one E and the sulfur atom corresponding to (e.g., the sulfur atom of) Cys13 of SEQ ID NO: 4 from another E. In some embodiments, when T is 2, the pairs of sulfur atoms are the sulfur atom corresponding to (e.g., the sulfur atom of) Cys10 of SEQ ID NO: 4 from one E and the sulfur atom corresponding to (e.g., the sulfur atom of) Cys10 of SEQ ID NO: 4 from another E and the sulfur atom corresponding to (e.g., the sulfur atom of) Cys13 of SEQ ID NO: 4 from one E and the sulfur atom corresponding to (e.g., the sulfur atom of) Cys13 of SEQ ID NO: 4 from another E.

In some embodiments, the conjugate has the structure:

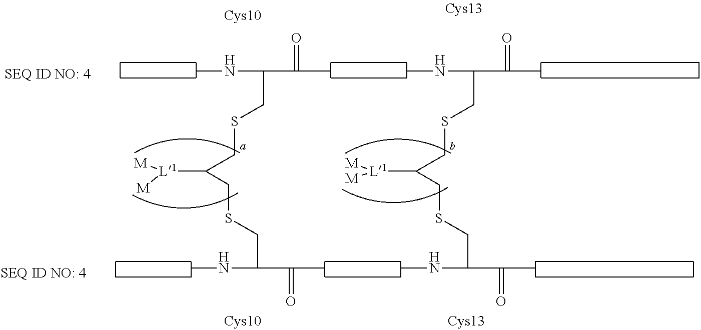

wherein each of a and b is, independently, 0 or 1 and wherein when a or b is 0, the two sulfur atoms form a disulfide bond. In some embodiments, a is 1 and b is 0. In some embodiments, a is 0 and b is 1. In some embodiments, a and b are 1.

In some embodiments, each M-L′¹ has the structure:

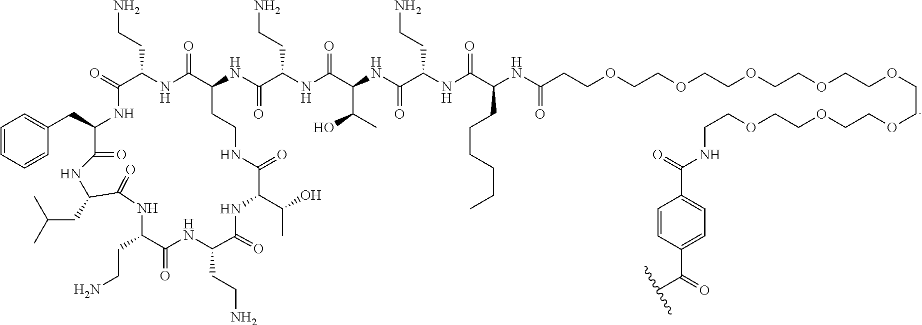 [SMILES: *C(=O)c1ccc(C(=O)NCCOCCOCCOCCOCCOCCOCCOCCOCCC(=O)N[C@@H](CCCCCC)C(=O)N[C@@H](CCN)C(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)cc1]

In some embodiments, each M-L′¹ has the structure:

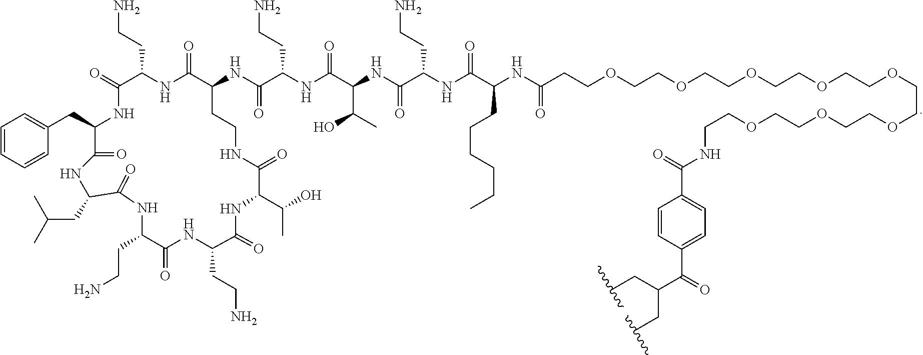 [SMILES: *CC(C*)C(=O)c1ccc(C(=O)NCCOCCOCCOCCOCCOCCOCCOCCOCCC(=O)N[C@@H](CCCCCC)C(=O)N[C@@H](CCN)C(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)cc1]

In some embodiments, each M-L′¹ has the structure:

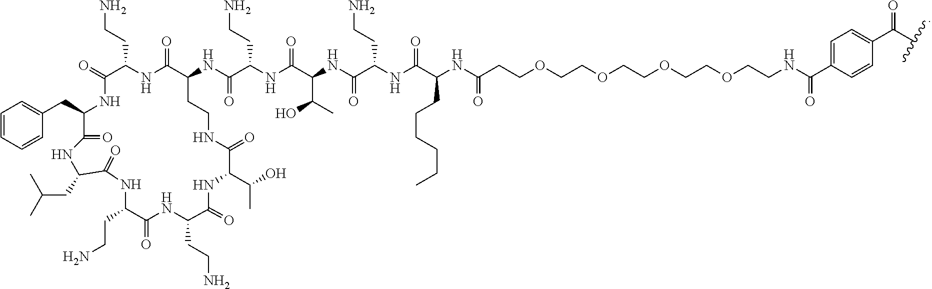 [SMILES: *C(=O)c1ccc(C(=O)NCCOCCOCCOCCOCCC(=O)N[C@@H](CCCCCC)C(=O)N[C@@H](CCN)C(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)cc1]

In some embodiments, each M-L′¹ has the structure:

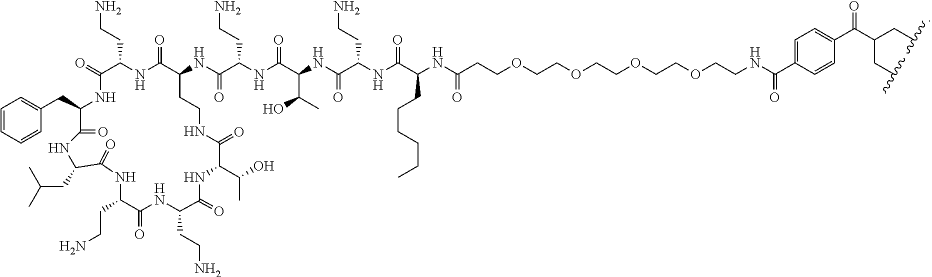 [SMILES: *CC(C*)C(=O)c1ccc(C(=O)NCCOCCOCCOCCOCCC(=O)N[C@@H](CCCCCC)C(=O)N[C@@H](CCN)C(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)cc1]

In some embodiments of these aspects, each E comprises the sequence

(SEQ ID NO: 8)
MVRSDKTHTCPPCPAPELLGGPSVFLFPPKPKDTLMISRT
PEVTCVVDVSHEDPEVKFNWYVDGVEVHNAKTKPREEQYN
STYRVVSVLTVLHQDWLNGKEYKCKVSNKALPAPIEKTIS
KAKGQPREPQVYTLPPSREEMTKNQVSLTCLVKGFYPSDI
AVEWESNGQPENNYKTTPPVLDSDGSFFLYSKLTVDKSRW
QQGNVFSCSVMHEALHNHYTQKSLSLSPGKHHHHHH.

In some embodiments, at least one of the pair of sulfur atoms is the sulfur atom corresponding to (e.g., the sulfur atom of) a hinge cysteine of SEQ ID NO: 8, i.e., Cys10 and/or Cys13. In some embodiments, the pair of sulfur atoms are the sulfur atoms corresponding to (e.g., the sulfur atoms of) Cys10 and Cys13 in SEQ ID NO: 8.

In some embodiments, the pair of sulfur atoms include one sulfur atom of a cysteine from each E, i.e., L′¹-M along with the sulfur atoms to which it is attached forms a bridge between two Fc domains (e.g., two Fc domains comprising the sequence of SEQ ID NO: 8). In some embodiments, the pair of sulfur atoms are the sulfur atom corresponding to (e.g., the sulfur atom of) Cys10 of SEQ ID NO: 8 from one E and the sulfur atom corresponding to (e.g., the sulfur atom of) Cys10 of SEQ ID NO: 8 from another E. In some embodiments, the pair of sulfur atoms are the sulfur atom corresponding to (e.g., the sulfur atom of) Cys13 of SEQ ID NO: 8 from one E and the sulfur atom corresponding to (e.g., the sulfur atom of) Cys13 of SEQ ID NO: 8 from another E. In some embodiments, when T is 2, the pairs of sulfur atoms are the sulfur atom corresponding to (e.g., the sulfur atom of) Cys10 of SEQ ID NO: 8 from one E and the sulfur atom corresponding to (e.g., the sulfur atom of) Cys10 of SEQ ID NO: 8 from another E and the sulfur atom corresponding to (e.g., the sulfur atom of) Cys13 of SEQ ID NO: 8 from one E and the sulfur atom corresponding to (e.g., the sulfur atom of) Cys13 of SEQ ID NO: 8 from another E.

In some embodiments, the conjugate has the structure:

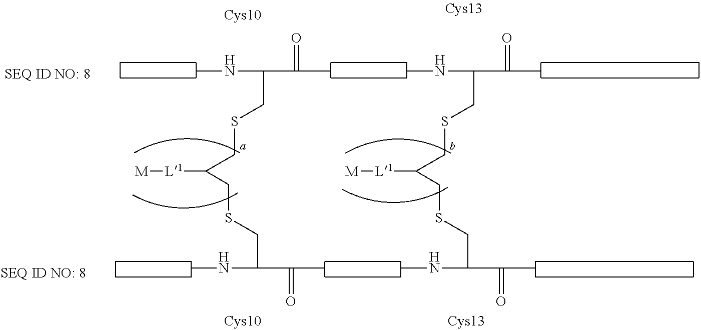

wherein each of a and b is, independently, 0 or 1 and wherein when a or b is 0, the two sulfur atoms form a disulfide bond. In some embodiments, a is 1 and b is 0. In some embodiments, a is 0 and b is 1. In some embodiments, a and b are 1.

In some embodiments of these aspects, the conjugate has the structure:

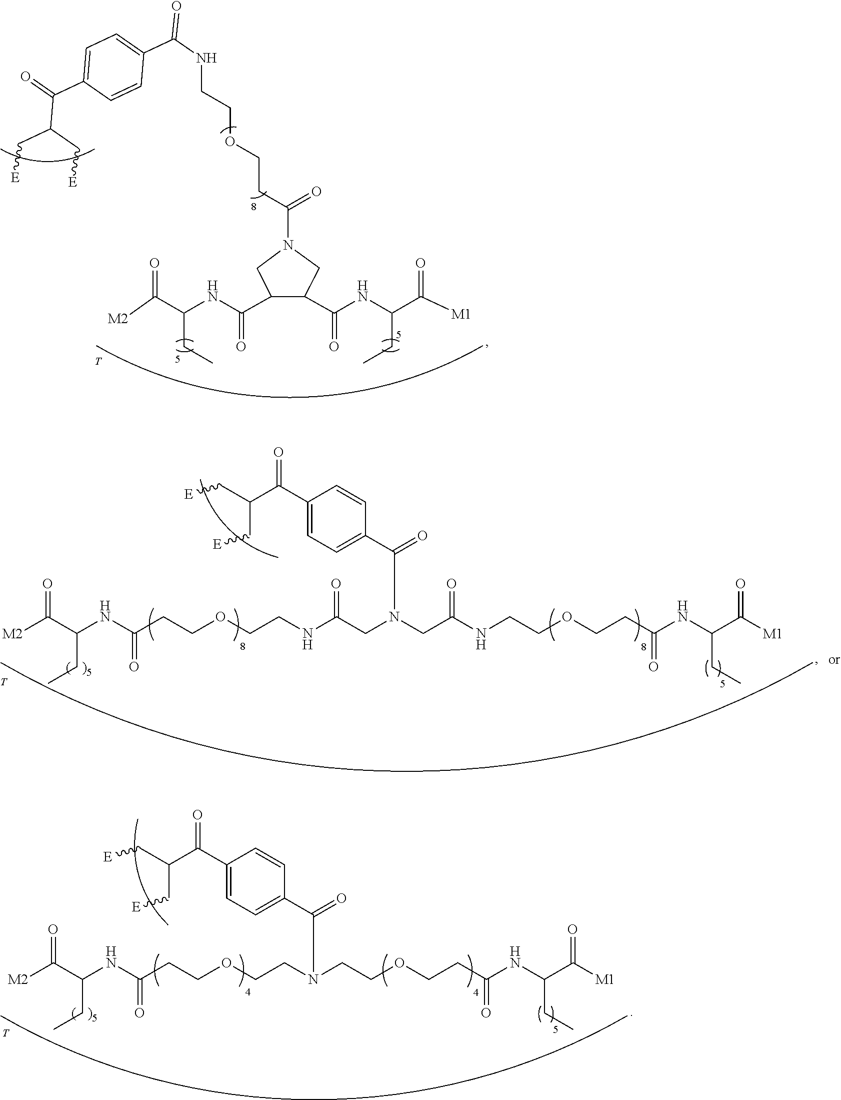

In some embodiments of these aspects, the conjugate has the structure:

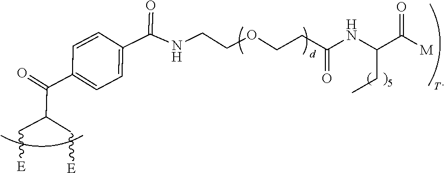

In another aspect, the invention also features a population of conjugates described in the previous two aspects, in which the average value of T is 1 to 5.

In another aspect, the invention features a conjugate described by formula (3):

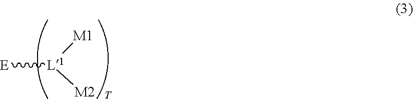

wherein each M1 includes a first cyclic heptapeptide including a linking nitrogen and each M2 includes a second cyclic heptapeptide including a linking nitrogen; E includes an Fc domain monomer (e.g., an Fc domain monomer having the sequence of any one of SEQ ID NOs: 1-14, 1-29, or 1-31); L′¹ in each M2-L′¹-M1 is a linker covalently attached to a sulfur atom of a hinge cysteine in E and to the linking nitrogen in each of M1 and M2; T is 1, 2, 3, 4, or 5, the squiggly line connected to the E indicates that each M2-L′¹-M1 is covalently attached to a sulfur atom of a hinge cysteine in E, or a pharmaceutically acceptable salt thereof. When T is greater than 1 (e.g., T is 2, 3, 4, or 5), each M2-L′¹-M1 may be independently selected (e.g., independently selected from any of the M2-L′¹-M1 structures described herein).

In some embodiments, each E includes an Fc domain monomer having the sequence of any one of SEQ ID NOs: 1-14, 1-29, or 1-31.

In some embodiments of these aspects, each E comprises the sequence

(SEQ ID NO: 10)
MVRSDKTHTCPPCPPC*KC*PAPELLGGPSVFLFPPKPKD
TLMISRTPEVTCVVVDVSHEDPEVKFNWYVDGVEVHNAKTK
PREEQYNSTYRVVSVLTVLHQDWLNGKEYKCKVSNKALPA
PIEKTISKAKGQPREPQVYTLPPSREEMTKNQVSLTCLVK
GFYPSDIAVEWESNGQPENNYKTTPPVLDSDGSFFLYSKL
TVDKSRWQQGNVFSCSVMHEALHNHYTQKSLSLSPGKHHH
HHH.

In some embodiments, at least one of the pair of sulfur atoms is the sulfur atom corresponding to (e.g., the sulfur atom of) a hinge cysteine of SEQ ID NO: 10, i.e., Cys10, Cys13, Cys16, or Cys18 of SEQ ID NO: 10. In some embodiments, the pair of sulfur atoms are the sulfur atoms corresponding to (e.g., the sulfur atoms of) Cys10 and Cys13 in SEQ ID NO: 10, Cys10 and Cys16 in SEQ ID NO: 10, Cys 30 and Cys18 in SEQ ID NO: 10, Cys13 and Cys 38 in SEQ ID NO: 10, Cys13 and Cys 38 in SEQ ID NO: 10, and/or Cys 36 and Cys 38 in SEQ ID NO: 10.

In some embodiments, when T is 2, the pair of sulfur atoms are the sulfur atoms corresponding to (e.g., the sulfur atoms of) Cys10 and Cys13 in SEQ ID NO: 10 and Cys 38 and Cys 38 in SEQ ID NO: 10.

In some embodiments, the pair of sulfur atoms include one sulfur atom of a cysteine from each E, i.e., L′¹-M along with the sulfur atoms to which it is attached forms a bridge between two Fc domains (e.g., two Fc domains comprising the sequence of SEQ ID NO: 10). In some embodiments, the pair of sulfur atoms are the sulfur atom corresponding to (e.g., the sulfur atom of) Cys10 of SEQ ID NO: 10 from one E and the sulfur atom corresponding to (e.g., the sulfur atom of) Cys10 of SEQ ID NO: 10 from another E. In some embodiments, the pair of sulfur atoms are the sulfur atom corresponding to (e.g., the sulfur atom of) Cys13 of SEQ ID NO: 10 from one E and the sulfur atom corresponding to (e.g., the sulfur atom of) Cys13 of SEQ ID NO: 10 from another E. In some embodiments, the pair of sulfur atoms are the sulfur atom corresponding to (e.g., the sulfur atom of) Cys16 of SEQ ID NO: 10 from one E and the sulfur atom corresponding to (e.g., the sulfur atom of) Cys16 of SEQ ID NO: 10 from another E. In some embodiments, the pair of sulfur atoms are the sulfur atom corresponding to (e.g., the sulfur atom of) Cys18 of SEQ ID NO: 10 from one E and the sulfur atom corresponding to (e.g., the sulfur atom of) Cys18 of SEQ ID NO: 10 from another E.

In some embodiments, when T is 2, the pairs of sulfur atoms are the sulfur atom corresponding to (e.g., the sulfur atom of) Cys10 of SEQ ID NO: 10 from one E and the sulfur atom corresponding to (e.g., the sulfur atom of) Cys10 of SEQ ID NO: 10 from another E and the sulfur atom corresponding to (e.g., the sulfur atom of) Cys13 of SEQ ID NO: 10 from one E and the sulfur atom corresponding to (e.g., the sulfur atom of) Cys13 of SEQ ID NO: 10 from another E. In some embodiments, when T is 2, the pairs of sulfur atoms are the sulfur atom corresponding to (e.g., the sulfur atom of) Cys10 of SEQ ID NO: 10 from one E and the sulfur atom corresponding to (e.g., the sulfur atom of) Cys10 of SEQ ID NO: 10 from another E and the sulfur atom corresponding to (e.g., the sulfur atom of) Cys16 of SEQ ID NO: 10 from one E and the sulfur atom corresponding to (e.g., the sulfur atom of) Cys16 of SEQ ID NO: 10 from another E. In some embodiments, when T is 2, the pairs of sulfur atoms are the sulfur atom corresponding to (e.g., the sulfur atom of) Cys10 of SEQ ID NO: 10 from one E and the sulfur atom corresponding to (e.g., the sulfur atom of) Cys10 of SEQ ID NO: 10 from another E and the sulfur atom corresponding to (e.g., the sulfur atom of) Cys18 of SEQ ID NO: 10 from one E and the sulfur atom corresponding to (e.g., the sulfur atom of) Cys18 of SEQ ID NO: 10 from another E.

In some embodiments, when T is 2, the pairs of sulfur atoms are the sulfur atom corresponding to (e.g., the sulfur atom of) Cys13 of SEQ ID NO: 10 from one E and the sulfur atom corresponding to (e.g., the sulfur atom of) Cys13 of SEQ ID NO: 10 from another E and the sulfur atom corresponding to (e.g., the sulfur atom of) Cys16 of SEQ ID NO: 10 from one E and the sulfur atom corresponding to (e.g., the sulfur atom of) Cys16 of SEQ ID NO: 10 from another E. In some embodiments, when T is 2, the pairs of sulfur atoms are the sulfur atom corresponding to (e.g., the sulfur atom of) Cys13 of SEQ ID NO: 10 from one E and the sulfur atom corresponding to (e.g., the sulfur atom of) Cys13 of SEQ ID NO: 10 from another E and the sulfur atom corresponding to (e.g., the sulfur atom of) Cys18 of SEQ ID NO: 10 from one E and the sulfur atom corresponding to (e.g., the sulfur atom of) Cys18 of SEQ ID NO: 10 from another E.

In some embodiments, when T is 2, the pairs of sulfur atoms are the sulfur atom corresponding to (e.g., the sulfur atom of) Cys16 of SEQ ID NO: 10 from one E and the sulfur atom corresponding to (e.g., the sulfur atom of) Cys16 of SEQ ID NO: 10 from another E and the sulfur atom corresponding to (e.g., the sulfur atom of) Cys18 of SEQ ID NO: 10 from one E and the sulfur atom corresponding to (e.g., the sulfur atom of) Cys18 of SEQ ID NO: 10 from another E.

In some embodiments, when T is 3, the pairs of sulfur atoms are the sulfur atom corresponding to (e.g., the sulfur atom of) Cys10 of SEQ ID NO: 10 from one E and the sulfur atom corresponding to (e.g., the sulfur atom of) Cys10 of SEQ ID NO: 10 from another E; the sulfur atom corresponding to (e.g., the sulfur atom of) Cys13 of SEQ ID NO: 10 from one E and the sulfur atom corresponding to (e.g., the sulfur atom of) Cys13 of SEQ ID NO: 10 from another E; and the sulfur atom corresponding to (e.g., the sulfur atom of) Cys16 of SEQ ID NO: 10 from one E and the sulfur atom corresponding to (e.g., the sulfur atom of) Cys16 of SEQ ID NO: 10 from another E. In some embodiments, when T is 3, the pairs of sulfur atoms are the sulfur atom corresponding to (e.g., the sulfur atom of) Cys10 of SEQ ID NO: 10 from one E and the sulfur atom corresponding to (e.g., the sulfur atom of) Cys10 of SEQ ID NO: 10 from another E; the sulfur atom corresponding to (e.g., the sulfur atom of) Cys13 of SEQ ID NO: 10 from one E and the sulfur atom corresponding to (e.g., the sulfur atom of) Cys13 of SEQ ID NO: 10 from another E; and the sulfur atom corresponding to (e.g., the sulfur atom of) Cys18 of SEQ ID NO: 10 from one E and the sulfur atom corresponding to (e.g., the sulfur atom of) Cys18 of SEQ ID NO: 10 from another E. In some embodiments, when T is 3, the pairs of sulfur atoms are the sulfur atom corresponding to (e.g., the sulfur atom of) Cys10 of SEQ ID NO: 10 from one E and the sulfur atom corresponding to (e.g., the sulfur atom of) Cys10 of SEQ ID NO: 10 from another E; the sulfur atom corresponding to (e.g., the sulfur atom of) Cys18 of SEQ ID NO: 10 from one E and the sulfur atom corresponding to (e.g., the sulfur atom of) Cys18 of SEQ ID NO: 10 from another E; and the sulfur atom corresponding to (e.g., the sulfur atom of) Cys16 of SEQ ID NO: 10 from one E and the sulfur atom corresponding to (e.g., the sulfur atom of) Cys16 of SEQ ID NO: 10 from another E. In some embodiments, when T is 3, the pairs of sulfur atoms are the sulfur atom corresponding to (e.g., the sulfur atom of) Cys13 of SEQ ID NO: 10 from one E and the sulfur atom corresponding to (e.g., the sulfur atom of) Cys13 of SEQ ID NO: 10 from another E; the sulfur atom corresponding to (e.g., the sulfur atom of) Cys18 of SEQ ID NO: 10 from one E and the sulfur atom corresponding to (e.g., the sulfur atom of) Cys18 of SEQ ID NO: 10 from another E; and the sulfur atom corresponding to (e.g., the sulfur atom of) Cys16 of SEQ ID NO: 10 from one E and the sulfur atom corresponding to (e.g., the sulfur atom of) Cys16 of SEQ ID NO: 10 from another E.

In some embodiments, when T is 3, the pairs of sulfur atoms are the sulfur atom corresponding to (e.g., the sulfur atom of) Cys10 of SEQ ID NO: 10 from one E and the sulfur atom corresponding to (e.g., the sulfur atom of) Cys10 of SEQ ID NO: 10 from another E; the sulfur atom corresponding to (e.g., the sulfur atom of) Cys13 of SEQ ID NO: 10 from one E and the sulfur atom corresponding to (e.g., the sulfur atom of) Cys13 of SEQ ID NO: 10 from another E; the sulfur atom corresponding to (e.g., the sulfur atom of) Cys16 of SEQ ID NO: 10 from one E and the sulfur atom corresponding to (e.g., the sulfur atom of) Cys16 of SEQ ID NO: 10 from another E; and the sulfur atom corresponding to (e.g., the sulfur atom of) Cys18 of SEQ ID NO: 10 from one E and the sulfur atom corresponding to (e.g., the sulfur atom of) Cys18 of SEQ ID NO: 10 from another E.

In some embodiments, the conjugate has the structure:

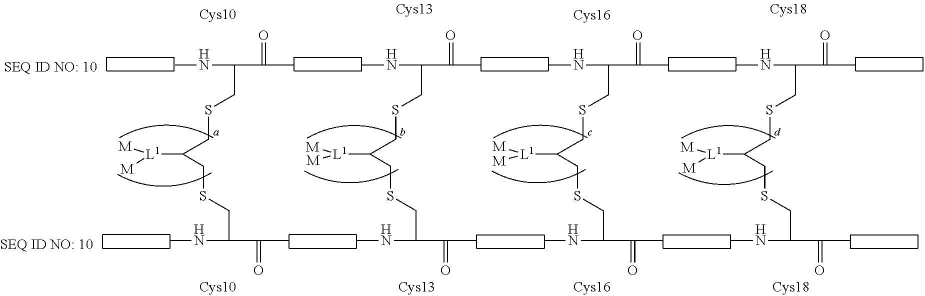

wherein each of a, b, c, and d is, independently, 0 or 1 and wherein when a, b, c, or d is 0, the two sulfur atoms form a disulfide bond.

In some embodiments, each M-L¹-M has the structure:

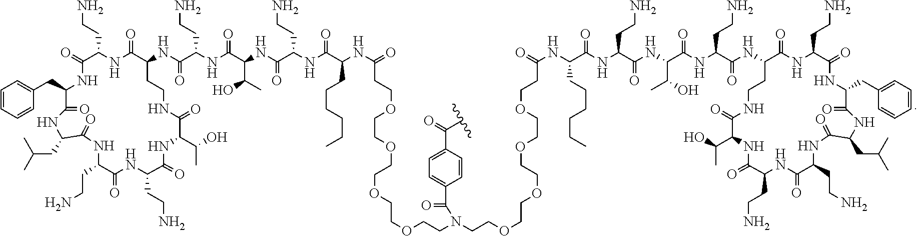 [SMILES: *C(=O)c1ccc(C(=O)N(CCOCCOCCOCCOCCC(=O)N[C@@H](CCCCCC)C(=O)N[C@@H](CCN)C(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)CCOCCOCCOCCOCCC(=O)N[C@@H](CCCCCC)C(=O)N[C@@H](CCN)C(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)cc1]

In some embodiments, each M-L¹-M has the structure:

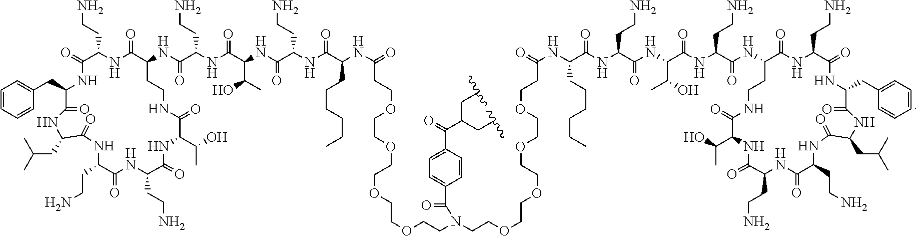 [SMILES: *CC(C*)C(=O)c1ccc(C(=O)N(CCOCCOCCOCCOCCC(=O)N[C@@H](CCCCCC)C(=O)N[C@@H](CCN)C(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)CCOCCOCCOCCOCCC(=O)N[C@@H](CCCCCC)C(=O)N[C@@H](CCN)C(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)cc1]

In some embodiments, each M-L-M has the structure:

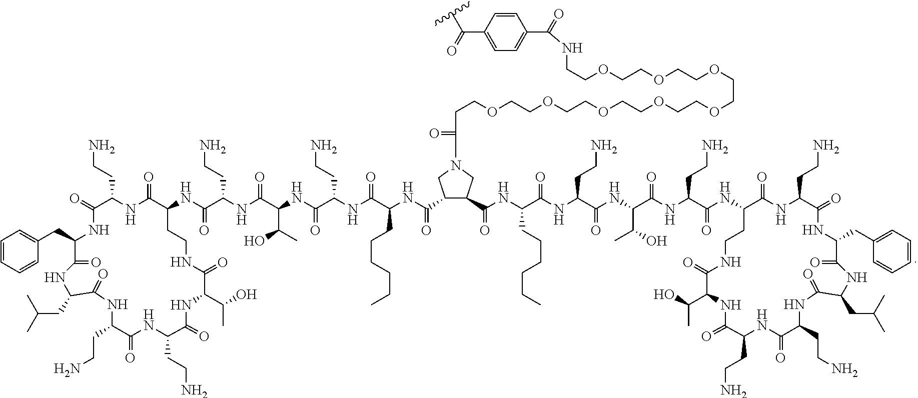 [SMILES: *C(=O)c1ccc(C(=O)NCCOCCOCCOCCOCCOCCOCCOCCOCCC(=O)N2C[C@@H](C(=O)N[C@@H](CCCCCC)C(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)[C@H](C(=O)N[C@@H](CCCCCC)C(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)C2)cc1]

In some embodiments, each M-L¹-M has the structure:

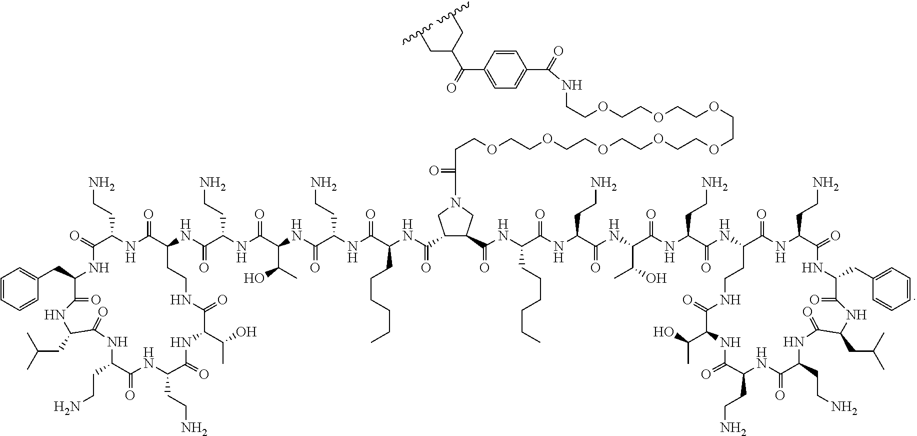 [SMILES: *CC(C*)C(=O)c1ccc(C(=O)NCCOCCOCCOCCOCCOCCOCCOCCOCCC(=O)N2C[C@@H](C(=O)N[C@@H](CCCCCC)C(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)[C@H](C(=O)N[C@@H](CCCCCC)C(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)C2)cc1]

In some embodiments, a is 1 and b, c, and d are 0. In some embodiments, a and b are 1 and c and d are 0. In some embodiments, a and c are 1 and b and d are 0. In some embodiments, a and d are 1 and b and c are 0. In some embodiments, a, b, and c are 1 and d is 0. In some embodiments, a, b, and d are 1 and c is 0. In some embodiments, a, c, and d are 1 and b is 0. In some embodiments, b and c are 1 and a and d are 0. In some embodiments, b and d are 1 and a and c are 0. In some embodiments, b, c, and d are 1 and a is 0. In some embodiments, c and d are 1 and a and b are 0. In some embodiments, a, b, c, and d are 1.

In some embodiments of these aspects, each E comprises the sequence

(SEQ ID NO: 4)
MVRSDKTHTCPPCPAPELLGGPSVFLFPPKPKDTLMISRT
PEVTCVVVVDVSHEDPEVKFNWYVDGVEVHNAKTKPREEQY
NSTYRVVSVLTVLHQDWLNGKEYKCKVSNKALPAPIEKTI
SKAKGQPREPQVYTLPPSREEMTKNQVSLTCLVKGFYPSD
IAVEWESNGQPENNYKTTPPVLDSDGSFFLYSKLTVDKSR
WQQGNVFSCSVMHEALHNHYTQKSLSLSPGK.

In some embodiments, at least one of the pair of sulfur atoms is the sulfur atom corresponding to (e.g., the sulfur atom of) a hinge cysteine of SEQ ID NO: 4, i.e., Cys10 and/or Cys13. In some embodiments, the pair of sulfur atoms are the sulfur atoms corresponding to (e.g., the sulfur atoms of) Cys10 and Cys13 in SEQ ID NO: 4.

In some embodiments, the pair of sulfur atoms include one sulfur atom of a cysteine from each E, i.e., L′¹-M along with the sulfur atoms to which it is attached forms a bridge between two Fc domains (e.g., two Fc domains comprising the sequence of SEQ ID NO: 4). In some embodiments, the pair of sulfur atoms are the sulfur atom corresponding to (e.g., the sulfur atom of) Cys10 of SEQ ID NO: 4 from one E and the sulfur atom corresponding to (e.g., the sulfur atom of) Cys10 of SEQ ID NO: 4 from another E. In some embodiments, the pair of sulfur atoms are the sulfur atom corresponding to (e.g., the sulfur atom of) Cys13 of SEQ ID NO: 4 from one E and the sulfur atom corresponding to (e.g., the sulfur atom of) Cys13 of SEQ ID NO: 4 from another E. In some embodiments, when T is 2, the pairs of sulfur atoms are the sulfur atom corresponding to (e.g., the sulfur atom of) Cys10 of SEQ ID NO: 4 from one E and the sulfur atom corresponding to (e.g., the sulfur atom of) Cys10 of SEQ ID NO: 4 from another E and the sulfur atom corresponding to (e.g., the sulfur atom of) Cys13 of SEQ ID NO: 4 from one E and the sulfur atom corresponding to (e.g., the sulfur atom of) Cys13 of SEQ ID NO: 4 from another E.

In some embodiments, the conjugate has the structure:

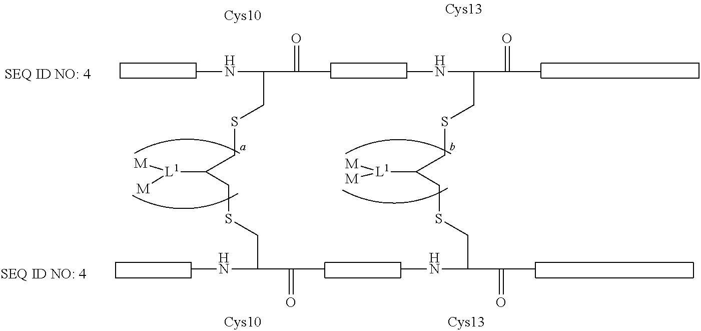

wherein each of a and b is, independently, 0 or 1 and wherein when a or b is 0, the two sulfur atoms form a disulfide bond. In some embodiments, a is 1 and b is 0. In some embodiments, a is 0 and b is 1. In some embodiments, a and b are 1.

In some embodiments of these aspects, each E comprises the sequence

(SEQ ID NO: 8)
MVRSDKTHTCPPCPAPELLGGPSVFLFPPKPKDTLMISRT
PEVTCVVVDVSHEDPEVKFNWYVDGVEVHNAKTKPREEQYN
STYRVVSVLTVLHQDWLNGKEYKCKVSNKALPAPIEKTISK
AKGQPREPQVYTLPPSREEMTKNQVSLTCLVKGFYPSDIA
VEWESNGQPENNYKTTPPVLDSDGSFFLYSKLTVDKSRWQ
QGNVFSCSVMHEALHNHYTQKSLSLSPGKHHHHHH.

In some embodiments, at least one of the pair of sulfur atoms is the sulfur atom corresponding to (e.g., the sulfur atom of) a hinge cysteine of SEQ ID NO: 8, i.e., Cys10 and/or Cys13. In some embodiments, the pair of sulfur atoms are the sulfur atoms corresponding to (e.g., the sulfur atoms of) Cys10 and Cys13 in SEQ ID NO: 8.

In some embodiments, the pair of sulfur atoms include one sulfur atom of a cysteine from each E, i.e., L′¹-M along with the sulfur atoms to which it is attached forms a bridge between two Fc domains (e.g., two Fc domains comprising the sequence of SEQ ID NO: 8). In some embodiments, the pair of sulfur atoms are the sulfur atom corresponding to (e.g., the sulfur atom of) Cys10 of SEQ ID NO: 8 from one E and the sulfur atom corresponding to (e.g., the sulfur atom of) Cys10 of SEQ ID NO: 8 from another E. In some embodiments, the pair of sulfur atoms are the sulfur atom corresponding to (e.g., the sulfur atom of) Cys13 of SEQ ID NO: 8 from one E and the sulfur atom corresponding to (e.g., the sulfur atom of) Cys13 of SEQ ID NO: 8 from another E. In some embodiments, when T is 2, the pairs of sulfur atoms are the sulfur atom corresponding to (e.g., the sulfur atom of) Cys10 of SEQ ID NO: 8 from one E and the sulfur atom corresponding to (e.g., the sulfur atom of) Cys10 of SEQ ID NO: 8 from another E and the sulfur atom corresponding to (e.g., the sulfur atom of) Cys13 of SEQ ID NO: 8 from one E and the sulfur atom corresponding to (e.g., the sulfur atom of) Cys13 of SEQ ID NO: 8 from another E.

In some embodiments, the conjugate has the structure:

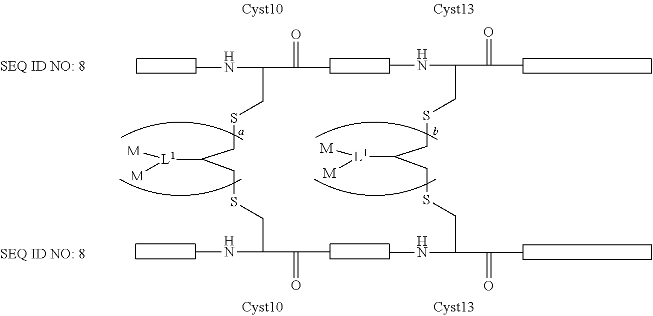

wherein each of a and b is, independently, 0 or 1 and wherein when a or b is 0, the two sulfur atoms form a disulfide bond. In some embodiments, a is 1 and b is 0. In some embodiments, a is 0 and b is 1. In some embodiments, a and b are 1.

In some embodiments, each M-L¹-M has the structure:

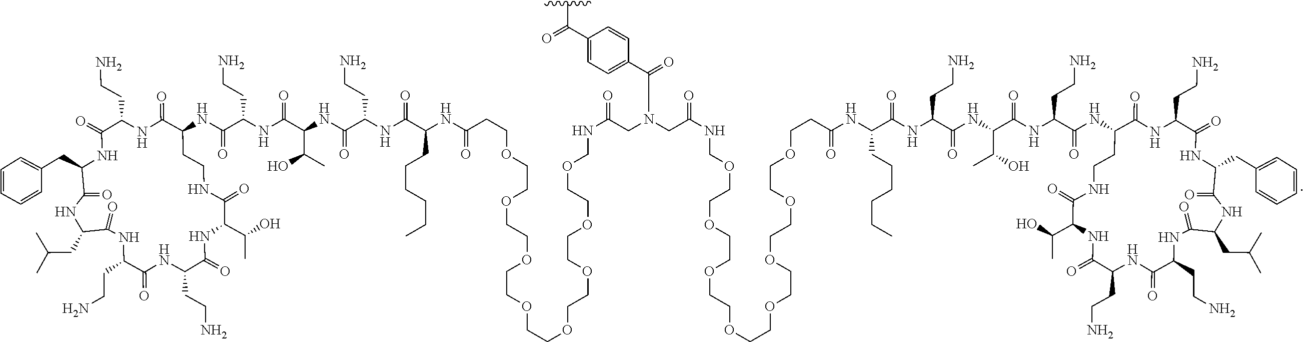

In some embodiments, each M-L¹-M has the structure:

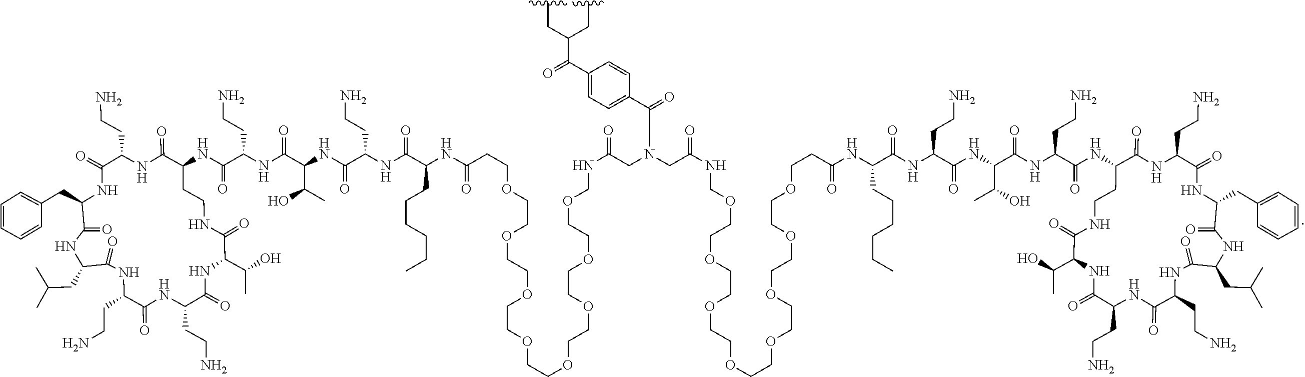

In another aspect, the invention features a conjugate described by formula (4):

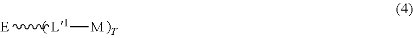

wherein each M includes a cyclic heptapeptide including a linking nitrogen; E includes an Fc domain monomer (e.g., an Fc domain monomer having the sequence of any one of SEQ ID NOs: 1-14, 1-29, or 1-31); L′¹ in each L′¹-M is a linker covalently attached to a sulfur atom of a hinge cysteine in E and to the linking nitrogen in M; T is 1, 2, 3, 4, or 5; the squiggly line connected to E indicates that each L′¹-M is covalently attached to the sulfur atom corresponding to (e.g., the sulfur atom of) the hinge cysteine in E, or a pharmaceutically acceptable salt thereof.

In some embodiments, each E includes an Fc domain monomer having the sequence of any one of SEQ ID Nos: 1-14, 1-29, or 1-31.

In some embodiments of these aspects, each E comprises the sequence

(SEQ ID NO: 4)
MVRSDKTHTCPPCPAPELLGGPSVFLFPPKPKDTLMISRT
PEVTCVVVDVSHEDPEVKFNWYVDGVEVHNAKTKPREEQYN
STYRVVSVLTVLHQDWLNGKEYKCKVSNKALPAPIEKTIS
KAKGQPREPQVYTLPPSREEMTKNQVSLTCLVKGFYPSDI
AVEWESNGQPENNYKTTPPVLDSDGSFFLYSKLTVDKSRW
OQGNVFSCSVMHEALHNHYTQKSLSLSPGK.

In some embodiments, at least one of the sulfur atoms is the sulfur atom corresponding to (e.g., the sulfur atom of) a hinge cysteine of SEQ ID NO: 4, i.e., Cys10 and/or Cys13. In some embodiments, the sulfur atoms is the sulfur atom corresponding to (e.g., the sulfur atom of) Cys10 in SEQ ID NO: 4. In some embodiments, the sulfur atom corresponding to (e.g., the sulfur atom of) Cys10 in SEQ ID NO: 4.

In some embodiments, the conjugate has the structure:

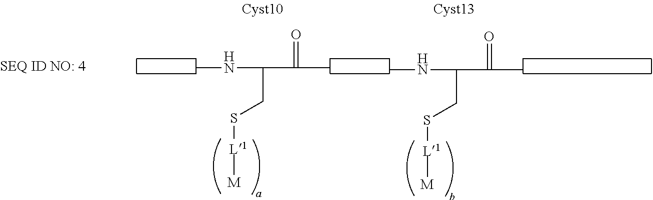

wherein each of a and b is, independently, 0 or 1 and wherein when a or b is 0, the sulfur atoms is a thiol. In some embodiments, a is 1 and b is 0. In some embodiments, a is 0 and b is 1. In some embodiments, a and b are 1.

In some embodiments, the conjugate has the structure:

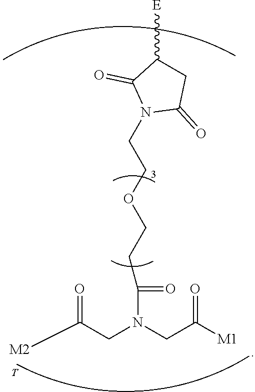

In some embodiments, the conjugate has the structure:

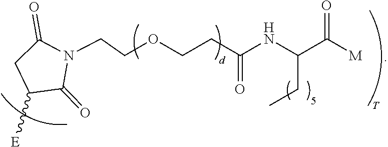

In another aspect, the invention features a population of conjugates described in the previous two aspects, wherein the average value of T is 1 to 5. In some embodiments, the average value of T is 1 to 2.

In another aspect, the invention features a conjugate described by formula (3):

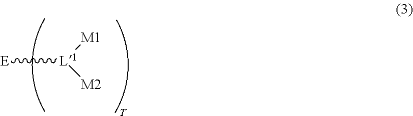

wherein each M1 includes a first cyclic heptapeptide including a linking nitrogen and each M2 includes a second cyclic heptapeptide including a linking nitrogen; E includes an Fc domain monomer (e.g., an Fc domain monomer having the sequence of any one of SEQ ID NOs: 1-14, 1-29, or 1-31); L′¹ in each M2-L′¹-M1 is a linker covalently attached to a nitrogen atom of a surface exposed lysine in E and to the linking nitrogen in each of M1 and M2; T is 1, 2, 3, 4, or 5, the squiggly line connected to the E indicates that each M2-L′¹-M1 is covalently attached to the nitrogen atom of a surface exposed lysine in E, or a pharmaceutically acceptable salt thereof.

In some embodiments, each E includes an Fc domain monomer having the sequence of any one of SEQ ID Nos: 1-14, 1-29, or 1-31.

In some embodiments of these aspects, each E comprises the sequence

(SEQ ID NO: 4)
MVRSDKTHTCPPCPAPELLGGPSVFLFPPKPKDTLMISRT
PEVTCVVVDVSHEDPEVKFNWYVDGVEVHNAKTKPREEQY
NSTYRVVSVLTVLHQDWLNGKEYKCKVSNKALPAPIEKTI
SKAKGQPREPQVYTLPPSREEMTKNQVSLTCLVKGFYPSD
IAVEWESNGQPENNYKTTPPVLDSDGSFFLYSKLTVDKSR
WOQGNVFSCSVMHEALHNHYTQKSLSLSPGK.

In some embodiments, at least one of the sulfur atoms is the sulfur atom of a hinge cysteine of SEQ ID NO: 4, i.e., Cys10 and/or Cys13. In some embodiments, the sulfur atom is sulfur atom corresponding to (e.g., the sulfur atom of) Cys10 in SEQ ID NO: 4. In some embodiments, the sulfur atom is the sulfur atom corresponding to (e.g., the sulfur atom of) Cys10 in SEQ ID NO: 4.

In some embodiments, the conjugate has the structure:

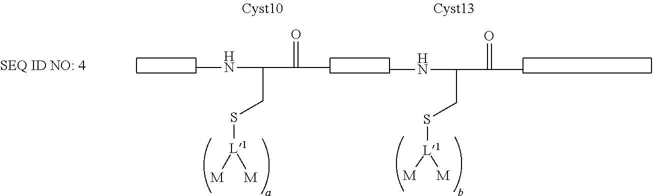

wherein each of a and b is, independently, 0 or 1 and wherein when a or b is 0, the sulfur atoms is a thiol. In some embodiments, a is 1 and b is 0. In some embodiments, a is 0 and b is 1. In some embodiments, a and b are 1.

In some embodiments, M-L′¹-M has the structure:

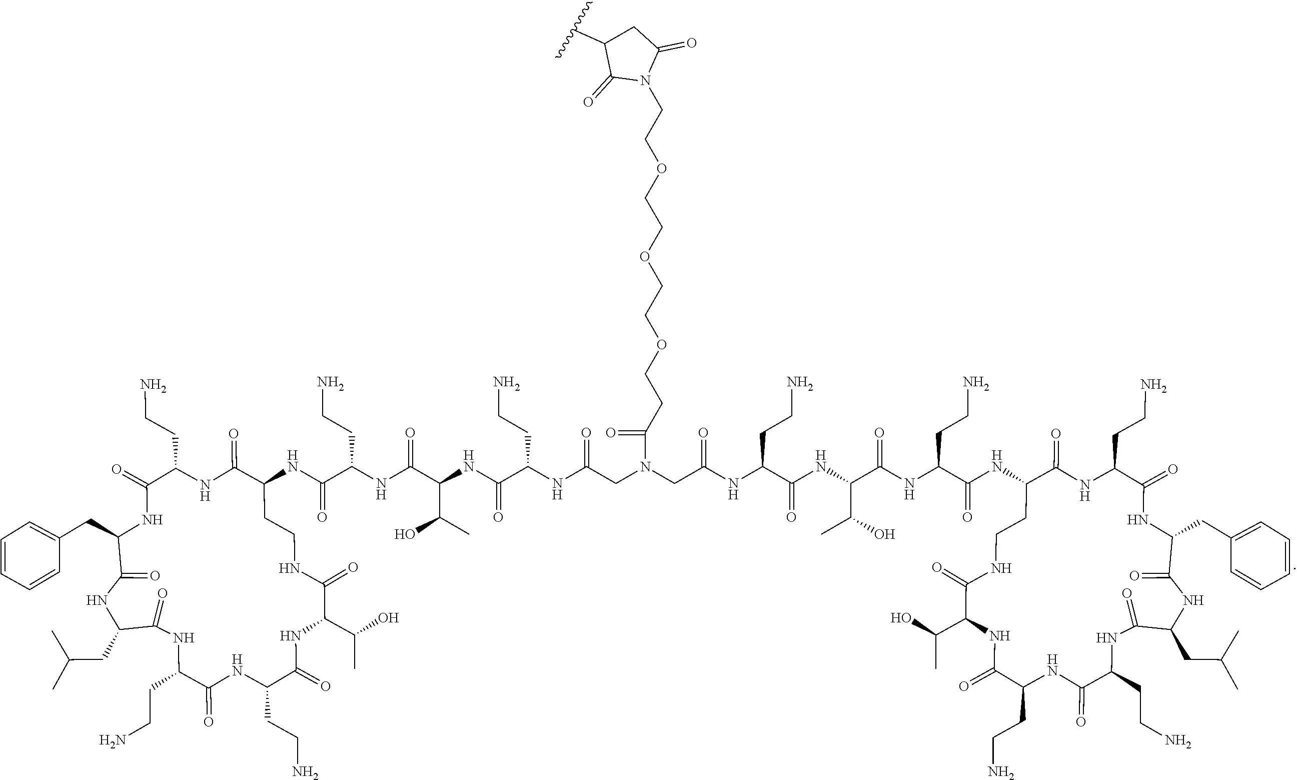 [SMILES: *C1CC(=O)N(CCOCCOCCOCCC(=O)N(CC(=O)N[C@@H](CCN)C(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)CC(=O)N[C@@H](CCN)C(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)C1=O]

In another aspect, the invention features, a conjugate described by formula (4):

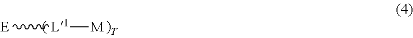

wherein each M includes a cyclic heptapeptide including a linking nitrogen; E includes an Fc domain monomer (e.g., an Fc domain monomer having the sequence ofany one of SEQ ID NOs: 1-14, 1-29, or 1-31); L′¹ in each L′¹-M is a linker covalently attached to a nitrogen atom of a surface exposed lysine in E and to the linking nitrogen in M; T is an integer from 1 to 11, the squiggly line connected to E indicates that each L′¹-M is covalently attached to the nitrogen atom of a surface exposed lysine in E, or a pharmaceutically acceptable salt thereof.

In some embodiments, each E includes an Fc domain monomer having the sequence of any one of SEQ ID NOs: 1-14, 1-29, or 1-31.

In some embodiments. E includes the sequence of

(SEQ ID NO: 10)
MVRSDKTHTCPPCPPC*KC*PAPELLGGPSVFLFPPKPKD
TLMISRTPEVTCVVVDVSHEDPEVKFNWYVDGVEVHNAKTK
PREEQYNSTYRVVSVLTVLHQDWLNGKEYKCKVSNKALPA
PIEKTISKAKGQPREPQVYTLPPSREEMTKNQVSLTCLVK
GFYPSDIAVEWESNGQPENNYKTTPPVLDSDGSFFLYSKL
TVDKSRWQQGNVFSCSVMHEALHNHYTQKSLSLSPGKHHH
HHH

In some embodiments of the previous two aspects, the nitrogen atom is the nitrogen of a surface exposed lysine, e.g., the nitrogen atom corresponding to (e.g. the nitrogen atom of) Lys35, Lys63, Lys77, Lys79, Lys106, Lys123, Lys129, Lys181, Lys203, Lys228, or Lys236 of SEQ ID NO: 10. In some embodiments, the nitrogen atom is the nitrogen atom corresponding to (e.g., the nitrogen atom of) Lys65, Lys79, Lys108, Lys230, and/or Lys238 of SEQ ID NO: 10.

In some embodiments, the conjugate has the structure:

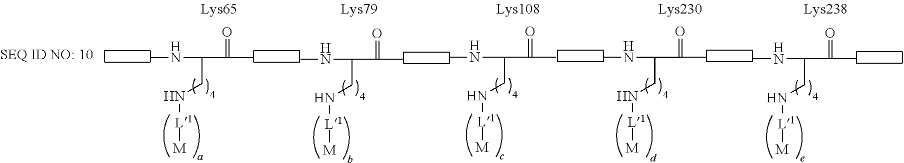

wherein each of a, b, c, d, and e is, independently, 0 or 1 and wherein when a, b, c, d, or e is 0, the two nitrogen atom is NH2. In some embodiments, a is 1 and b, c, d, and e are 0. In some embodiments, b is 1 and a, c, d, and e are 0. In some embodiments, c is 1 and a, b, d, and e are 0. In some embodiments, d is 1 and a, b, c, and e are 0. In some embodiments, e is 1 and a, b, c, and d are 0. In some embodiments, a and b are 1 and c, d, and e are 0. In some embodiments, a and c are 1 and b, d, and e are 0. In some embodiments, a and d are 1 and b, c, and e are 0. In some embodiments, a and e are 1 and b, c, and d are 0. In some embodiments, b and c are 1 and a, d, and e are 0. In some embodiments, b and d are 1 and a, c, and e are 0. In some embodiments, b and e are 1 and a, c, and d are 0. In some embodiments, c and d are 1 and a, b, and e are 0. In some embodiments, c and e are 1 and a, b, and d are 0. In some embodiments, d and e are 1 and a, b, and c are 0. In some embodiments, a, b, and c are 1 and d and e are 0. In some embodiments, a, b, and d are 1 and c and e are 0. In some embodiments, a, b, and e are 1 and c and d are 0. In some embodiments, a, c, and d are 1 and b and e are 0. In some embodiments, a, c, and e are 1 and b and d are 0. In some embodiments, a, d, and e are 1 and b and c are 0. In some embodiments, b, c, and d are 1 and a and e are 0. In some embodiments, b, d, and e are 1 and a and c are 0. In some embodiments, c, d, and e are 1 and a and b are 0.

In some embodiments, the conjugate has the structure

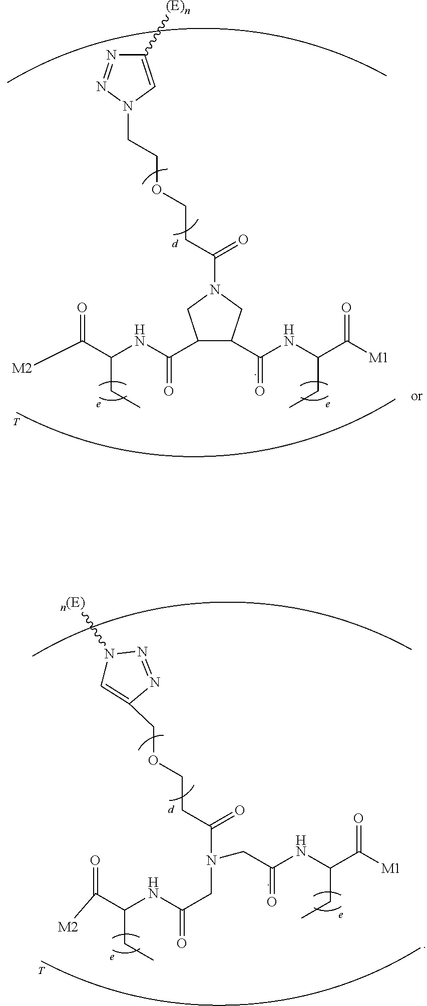

wherein each e and each d is independently selected from an integer of 1 to 10 (e.g., 1, 2, 3, 4, 5, 6, 7, 8, 9, or 10).

In some embodiments, the conjugate has the structure

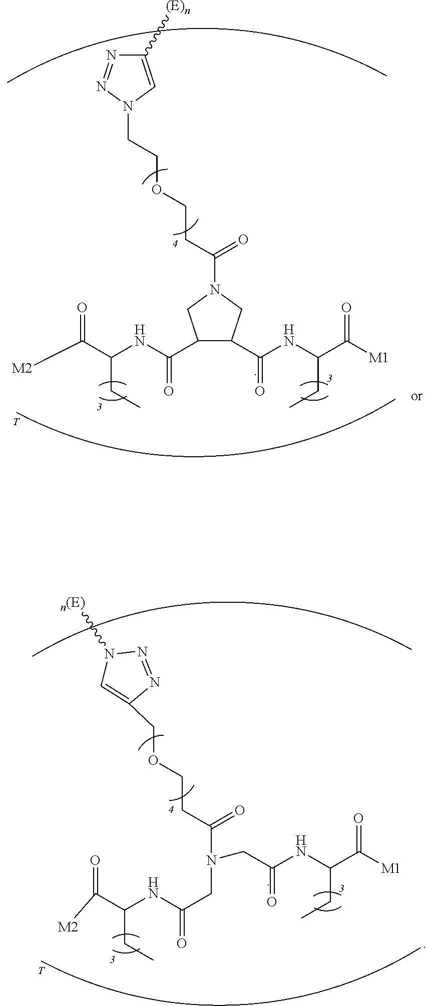

In some embodiments, the conjugate has the structure

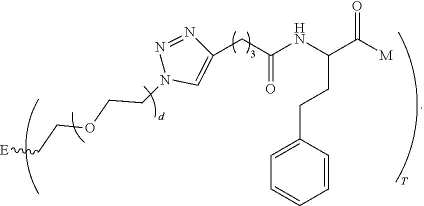

In another aspect, the invention features a population of conjugates described in the previous two aspects, wherein the average value of T is 1 to 5. In some embodiments, the average value of T is 1 to 2.

In some embodiments of the conjugates described herein, the conjugate forms a homodimer including an Fc domain.

In some embodiments of the conjugates described herein, E homodimerizes with another E to form an Fc domain.

In another aspect, the invention features a conjugate including (i) a first cyclic heptapeptide; (ii) a second cyclic heptapeptide; (iii) an Fc domain monomer or an Fc domain; and (iv) a linker covalently attached to the first cyclic heptapeptide, the second cyclic heptapeptide, and the Fc domain monomer or the Fc domain.

In another aspect, the invention features a conjugate described by formula (D-Ia):

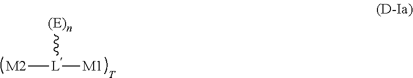

wherein each M1 includes a first cyclic heptapeptide including a linking nitrogen and each M2 includes a second cyclic heptapeptide including a linking nitrogen; L′ in each M2-L′-M1 is a linker covalently attached to the Fc domain monomer and to the linking nitrogen in each of M1 and M2; each E is an Fc domain monomer; n is 1 or 2; T is an integer from 1 to 20, e.g., 1 to 15, or a pharmaceutically acceptable salt thereof. When T is greater than 1 (e.g., T is 2, 3, 4, 5, 6, 7, 8, 9, 10, 11, 12, 13, 14, 15, 16, 17, 18, 19, or 20), each M2-L′-M1 may be independently selected (e.g., independently selected from any of the M2-L′-M1 structures described herein).

In some embodiments, E includes the sequence of

(SEQ ID NO: 10)
MVRSDKTHTCPPCPPC*KC*PAPELLGGPSVFLFPPKPKD
TLMISRTPEVTCVVDVSHEDPEVKFNWYVDGVEVHNAKTK
PREEQYNSTYRVVSVLTVLHQDWLNGKEYKCKVSNKALPA
PIEKTISKAKGQPREPQVYTLPPSREEMTKNQVSLTCLVK
GFYPSDIAVEWESNGQPENNYKTTPPVLDSDGSFFLYSKL
TVDKSRWQQGNVFSCSVMHEALHNHYTQKSLSLSPGKHHH
HHH

In some embodiments of the previous two aspects, the nitrogen atom is the nitrogen of a surface exposed lysine, e.g., the nitrogen atom corresponding to (e.g., the nitrogen atom of) Lys35, Lys63, Lys77, Lys79, Lys106, Lys123, Lys129, Lys181, Lys203, Lys228, or Lys236 of SEQ ID NO: 10. In some embodiments, the nitrogen atom is the nitrogen atom corresponding to (e.g., the nitrogen atom of) Lys65, Lys79, Lys108, Lys230, and/or Lys238 of SEQ ID NO:10.

In some embodiments, the conjugate has the structure:

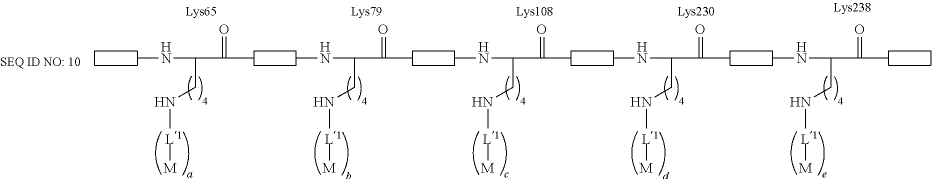

wherein each of a, b, c, d, and e is, independently, 0 or 1 and wherein when a, b, c, d, or e is 0, the two nitrogen atom is NH2. In some embodiments, a is 1 and b, c, d, and e are 0. In some embodiments, b is 1 and a, c, d, and e are 0. In some embodiments, c is 1 and a, b, d, and e are 0. In some embodiments, d is 1 and a, b, c, and e are 0. In some embodiments, e is 1 and a, b, c, and d are 0. In some embodiments, a and b are 1 and c, d, and e are 0. In some embodiments, a and c are 1 and b, d, and e are 0. In some embodiments, a and d are 1 and b, c, and e are 0. In some embodiments, a and e are 1 and b, c, and d are 0. In some embodiments, b and c are 1 and a, d, and e are 0. In some embodiments, b and d are 1 and a, c, and e are 0. In some embodiments, b and e are 1 and a, c, and d are 0. In some embodiments, c and d are 1 and a, b, and e are 0. In some embodiments, c and e are 1 and a, b, and d are 0. In some embodiments, d and e are 1 and a, b, and c are 0. In some embodiments, a, b, and c are 1 and d and e are 0. In some embodiments, a, b, and d are 1 and c and e are 0. In some embodiments, a, b, and e are 1 and c and d are 0. In some embodiments, a, c, and d are 1 and b and e are 0. In some embodiments, a, c, and e are 1 and b and d are 0. In some embodiments, a, d, and e are 1 and b and c are 0. In some embodiments, b, c, and d are 1 and a and e are 0. In some embodiments, b, d, and e are 1 and a and c are 0. In some embodiments, c, d, and e are 1 and a and b are 0.

In some embodiments, L′¹-M has the structure:

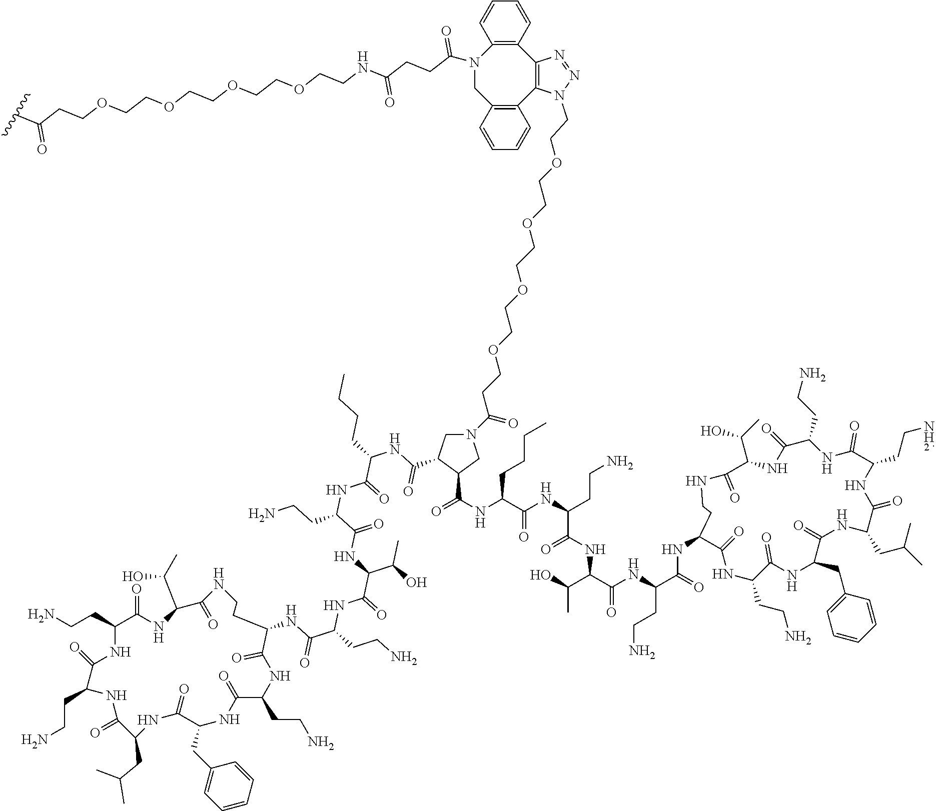

In some embodiments of this aspect, when n is 2. E dimerizes to form an Fc domain, and the conjugate is described by formula (D-Ib):

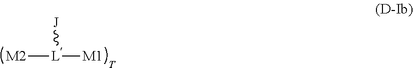

wherein J is an Fc domain; and T is an integer from 2 to 20, e.g., 2 to 15, or a pharmaceutically acceptable salt thereof.

In some embodiments of this aspect, L′ in each M2-L′-M1 is described by formula (D-L):

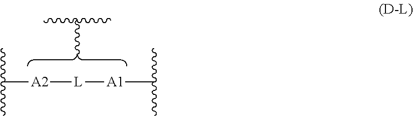

wherein L is a remainder of L′; A1 is a 1-5 amino acid peptide covalently attached to the linking nitrogen in each M1 or is absent; and A2 is a 1-5 amino acid peptide covalently attached to the linking nitrogen in each M2 or is absent.

In some embodiments of this aspect, the conjugate is described by formula (D-IIa):

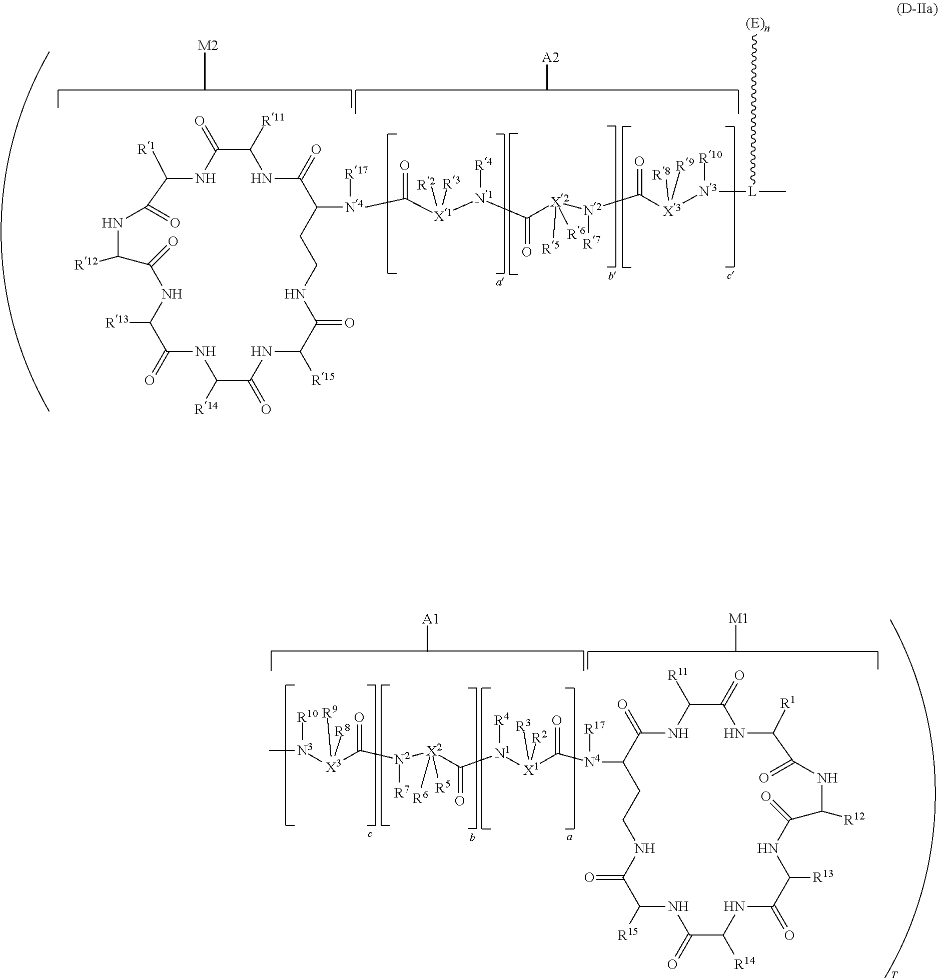

wherein L is a remainder of L′; each of R¹, R¹², R′¹, and R′¹² is, independently, a lipophilic moiety, a polar moiety, or H; each of R¹¹, R¹³, R¹⁴, R′¹¹, R′¹³, and R′¹⁴ is, independently, optionally substituted C1-C5 alkamino, a polar moiety, a positively charged moiety, or H; each of R¹⁵ and R′¹⁵ is, independently, a lipophilic moiety or a polar moiety; each of R², R³, R⁴, R⁵, R⁶, R⁷, R⁸, R⁹, R¹⁰, R′², R′³, R′⁴, R′⁵, R′⁶, R′⁷, R′⁸, R′⁹, and R′¹⁰ is, independently, a lipophilic moiety, a positively charged moiety, a polar moiety, H, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; or two R groups on the same or adjacent atoms join to form an optionally substituted 3-8 membered ring (e.g., 5-8 membered ring); each or R¹⁷ and R′¹⁷ is, independently. H, C1-C3 alkyl, or C2-C3 alkamino; each of a′, b′, c′, a, b, and c is, independently, 0 or 1; each of N¹, N², N³, N⁴, N′¹, N′², N′³, and N′⁴ is a nitrogen atom; and each of X¹, X², X³, X′¹, X′², and X′³ is, independently, a carbon atom or a nitrogen atom, wherein if X¹ is a nitrogen atom then R³ is absent, if X′¹ is a nitrogen atom then R′³ is absent, if X² is a nitrogen atom then R⁵ is absent, if X′² is a nitrogen atom then R′⁵ is absent, if X³ is a nitrogen atom then R⁹ is absent, and if X′³ is a nitrogen atom then R′⁹ is absent; or a pharmaceutically acceptable salt thereof.

In some embodiments, the conjugate includes at least one optionally substituted 3-8 membered ring (e.g., 5-8 membered ring) formed by joining (i) R², R³, and X¹; (ii) R³, R⁴, N¹, and X¹; (iii) R⁵, R⁶, and X²; (iv) R⁶, R⁷, N², and X²; (v) R⁸, R⁹, and X³; (vi) R⁹, R¹⁰, N³, and X³; (F(Mvii) R′², R′³, and X′¹; (viii) R′³, R′⁴, N′¹, and X′¹; (ix) R′⁵, R′⁶, and X′²; (x) R′⁶, R′⁷, N′², and X′²; (xi) R′⁸, R′⁹, and X′³; or (xii) R′⁹, R′¹⁰, N′³, and X′³.

In some embodiments of this aspect, the conjugate is described by formula (D-IIb):

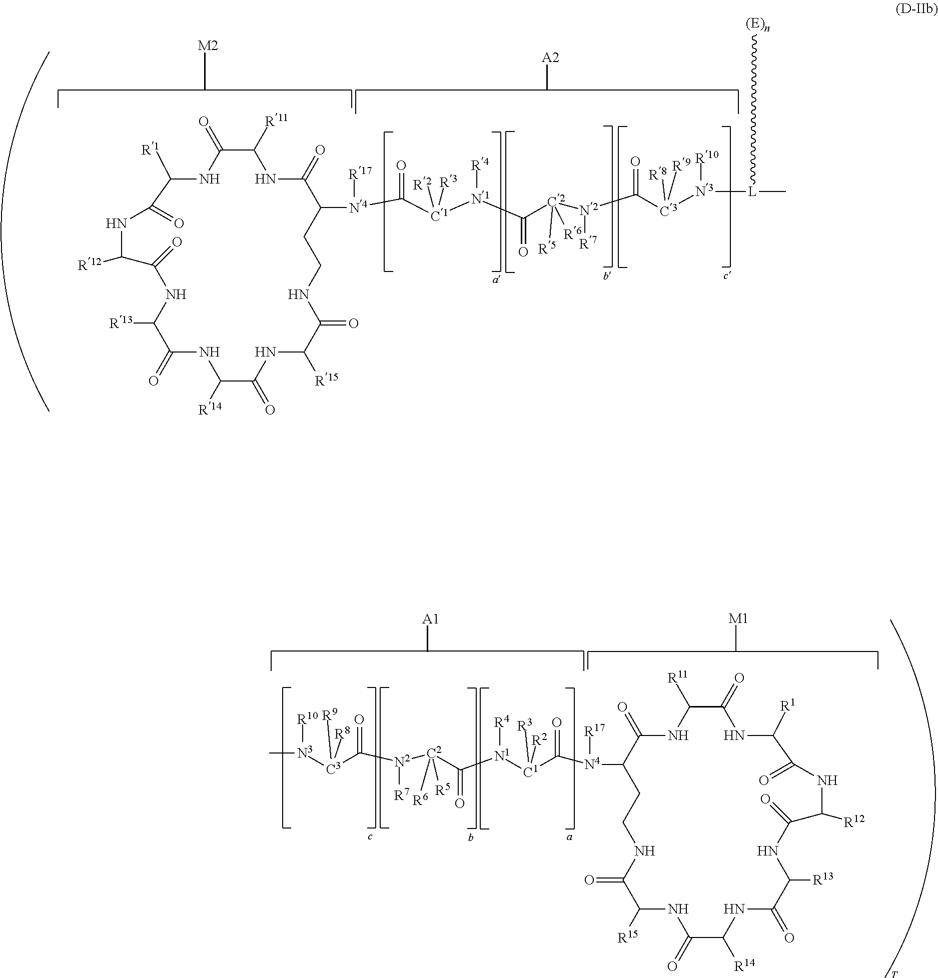

wherein L is a remainder of L′; each of R¹, R¹², R′¹, and R′¹² is, independently, a lipophilic moiety, a polar moiety, or H; each of R¹¹, R¹³, R¹⁴, R′¹¹, R′¹³, and R′¹⁴ is, independently, optionally substituted C1-C5 alkamino, a polar moiety, a positively charged moiety, or H; each of R′¹⁵ and R′¹⁵ is, independently, a lipophilic moiety or a polar moiety; each of R², R³, R⁴, R⁵, R⁶, R⁷, R⁸, R⁹, R¹⁰, R′², R′³, R′⁴, R′⁵, R′⁶, R′⁷, R′⁸, R′⁹, and R′¹⁰ is, independently, a lipophilic moiety, a positively charged moiety, a polar moiety, H, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; or two R groups on the same or adjacent atoms join to form an optionally substituted 3-8 membered ring (e.g. 5-8 membered ring); each or R¹⁷ and R′¹⁷ is, independently, H, C1-C3 alkyl, or C2-C3 alkamino; each of a′, b′, c′, a, b, and c is, independently, 0 or 1; each of N¹, N², N³, N⁴, N′¹, N′², N′³, and N′⁴ is a nitrogen atom; each of C¹, C², C³, C′¹, C′², and C′³ is a carbon atom, or a pharmaceutically acceptable salt thereof.

In some embodiments, the conjugate includes at least one optionally substituted 3-8 membered ring (e.g., 5-8 membered ring) formed by joining (i) R², R³, and C¹; (ii) R³, R⁴, N¹, and C¹; (iii) R⁵, R⁶, and C²; (iv) R⁶, R⁷, N², and C²; (v) R⁸, R⁹, and C³; (vi) R⁹, R¹⁰, N³, and C³; (vii) R′², R′³, and C′¹; (viii) R′³, R′⁴, N′¹, and C′¹; (ix) R′⁵, R′⁶, and C′²; (x) R′⁶, R′⁷, N′², and C′²; (xi) R′⁸, R′⁹, and C′³; or (xii) R′⁹, R′¹⁰, N′³, and C′³.

In some embodiments, the conjugate is described by formula (D-IIc):

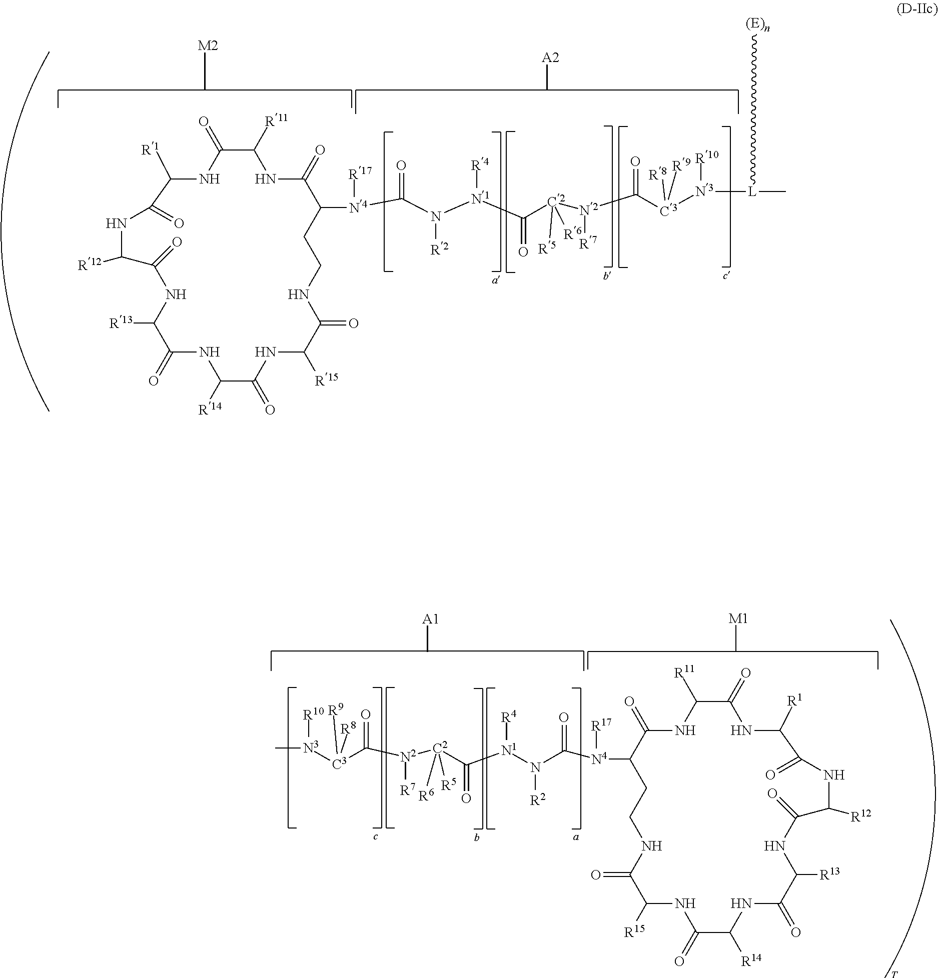

wherein L is a remainder of L′; each of R¹, R¹², R′¹, and R′¹² is, independently, a lipophilic moiety, a polar moiety, or H; each of R¹¹, R¹³, R¹⁴, R′¹¹, R′¹³, and R′¹⁴ is, independently, optionally substituted C1-C5 alkamino, a polar moiety, a positively charged moiety, or H; each of R¹⁵ and R′¹⁵ is, independently, a lipophilic moiety or a polar moiety; each of R², R⁴, R⁵, R⁸, R⁷, R⁸, R⁹, R¹⁰, R′², R′⁴, R′⁵, R′⁶, R′⁷, R′⁸, R′⁹, and R′¹⁰ is, independently, a lipophilic moiety, a positively charged moiety, a polar moiety, H, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; or two R groups on the same or adjacent atoms join to form an optionally substituted 3-8 membered ring (e.g., 5-8 membered ring); each or R¹⁷ and R′¹⁷ is, independently, H, C1-C3 alkyl, or C2-C3 alkamino; each of a′, b′, c′, a, b, and c is, independently, 0 or 1; each of N¹, N², N³, N⁴, N′¹, N′², N′³, and N′⁴ is a nitrogen atom; and each of C², C³, C′², and C′³ is a carbon atom, or a pharmaceutically acceptable salt thereof.

In some embodiments, the conjugate is described by formula (D-IId):

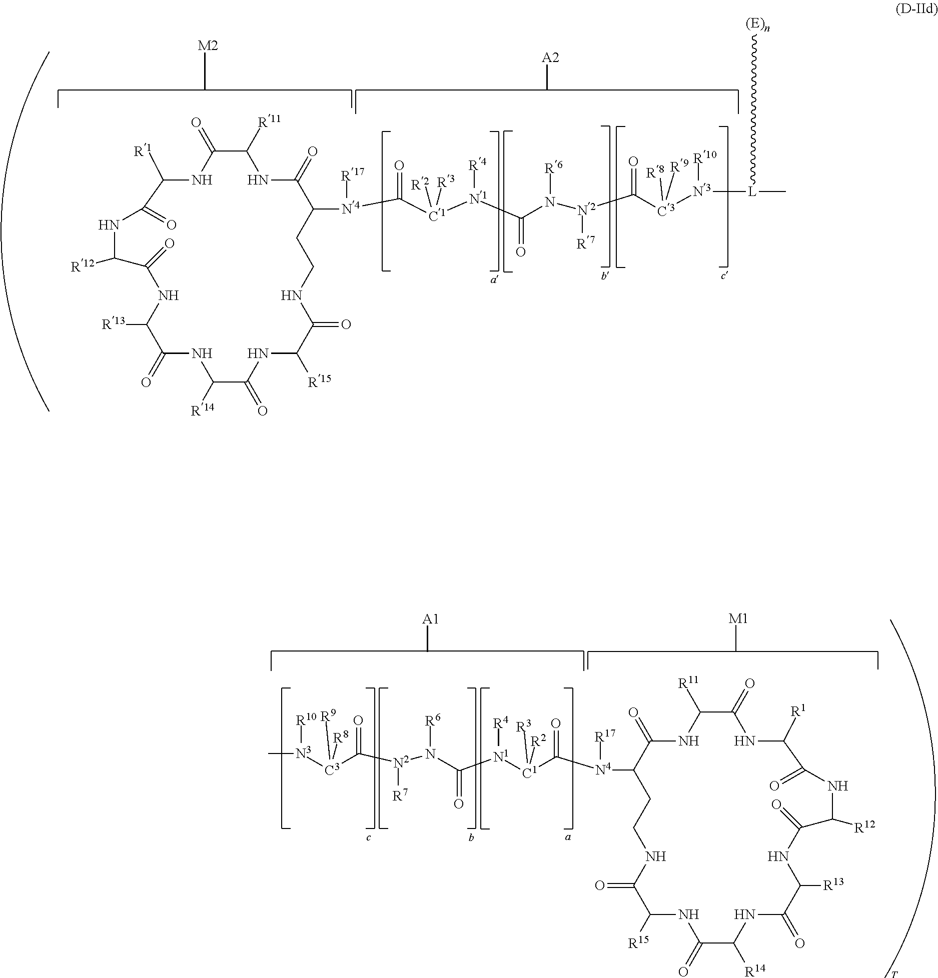

wherein L is a remainder of L′; each of R¹, R¹², R′¹, and R′¹² is, independently, a lipophilic moiety, a polar moiety, or H; each of R¹¹, R¹³, R¹⁴, R′¹¹, R′¹³, and R′¹⁴ is, independently, optionally substituted C1-C5 alkamino, a polar moiety, a positively charged moiety, or H; each of R¹⁵ and R′¹⁵ is, independently, a lipophilic moiety or a polar moiety; each of R², R³, R⁴, R⁶, R⁷, R⁸, R⁹, R¹⁰, R′², R′³, R′⁴, R′⁵, R′⁷, R′⁸, R′⁹, and R′¹⁰ is, independently, a lipophilic moiety, a positively charged moiety, a polar moiety, H, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; or two R groups on the same or adjacent atoms join to form an optionally substituted 3-8 membered ring (e.g., 5-8 membered ring); each or R¹⁷ and R′¹⁷ is, independently, H, C1-C3 alkyl, or C2-C3 alkamino; each of a′, b′, c′, a, b, and c is, independently, 0 or 1; each of N¹, N², N³, N⁴, N′¹, N′², N′³, and N′⁴ is a nitrogen atom; and each of C¹, C³, C′¹, and C′³ is a carbon atom, or a pharmaceutically acceptable salt thereof.

In some embodiments, the conjugate is described by formula (D-IIe):

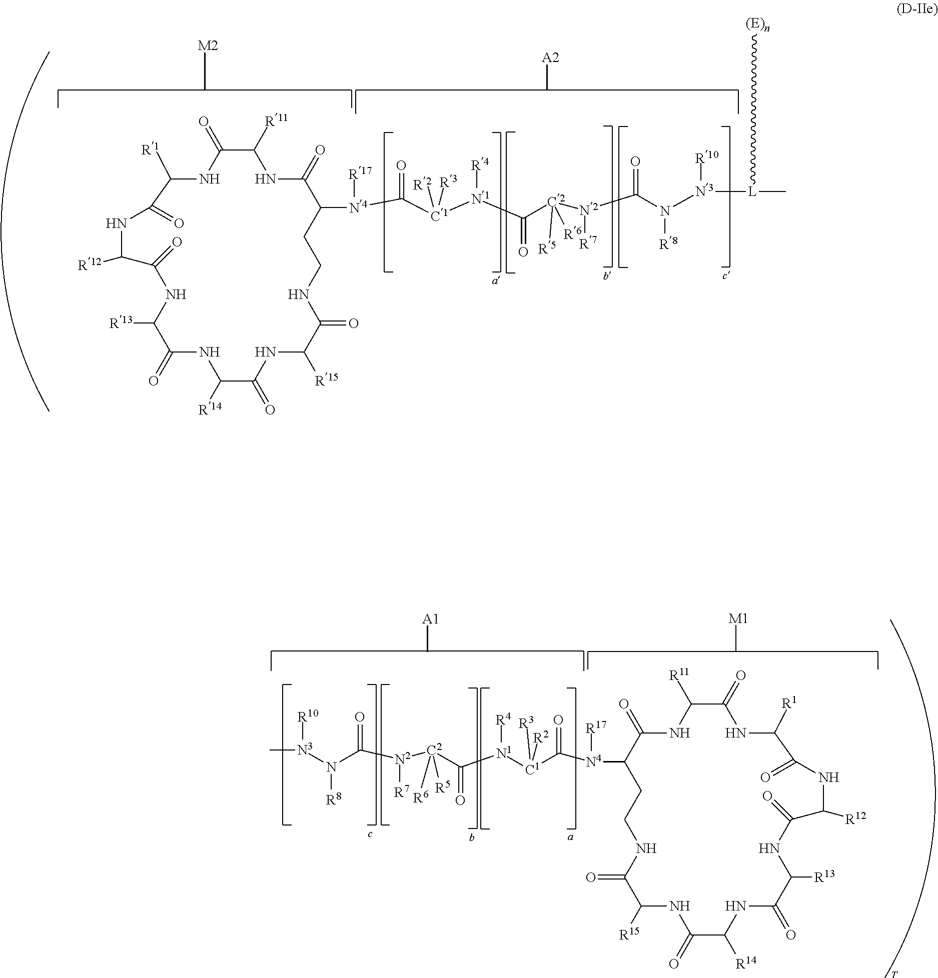

wherein L is a remainder of L′; each of R¹, R¹², R′¹, and R′¹² is, independently, a lipophilic moiety, a polar moiety, or H; each of R¹¹, R¹³, R¹⁴, R′¹¹, R′¹³, and R′¹⁴ is, independently, optionally substituted C1-C5 alkamino, a polar moiety, a positively charged moiety, or H; each of R¹⁵ and R′¹⁵ is, independently, a lipophilic moiety or a polar moiety; each of R², R³, R⁴, R⁵, R⁶, R⁷, R⁸, R¹⁰, R′², R′³, R′⁴, R′⁵, R′⁶, R′⁷, R′⁸, and R′¹⁰ is, independently, a lipophilic moiety, a positively charged moiety, a polar moiety. H, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C8-C35 alkaryl, or optionally substituted C8-C35 heteroalkaryl; or two R groups on the same or adjacent atoms join to form an optionally substituted 3-8 membered ring (e.g., 5-8 membered ring); each or R¹⁷ and R′¹⁷ is, independently, H, C1-C3 alkyl, or C2-C3 alkamino; each of a′, b′, c′, a, b, and c is, independently, 0 or 1; each of N¹, N², N³, N⁴, N′¹, N′², N′³, and N′⁴ is a nitrogen atom; and each of C¹, C², C′¹, and C′² is a carbon atom, or a pharmaceutically acceptable salt thereof.

In some embodiments, the conjugate is described by formula (D-IIIa):

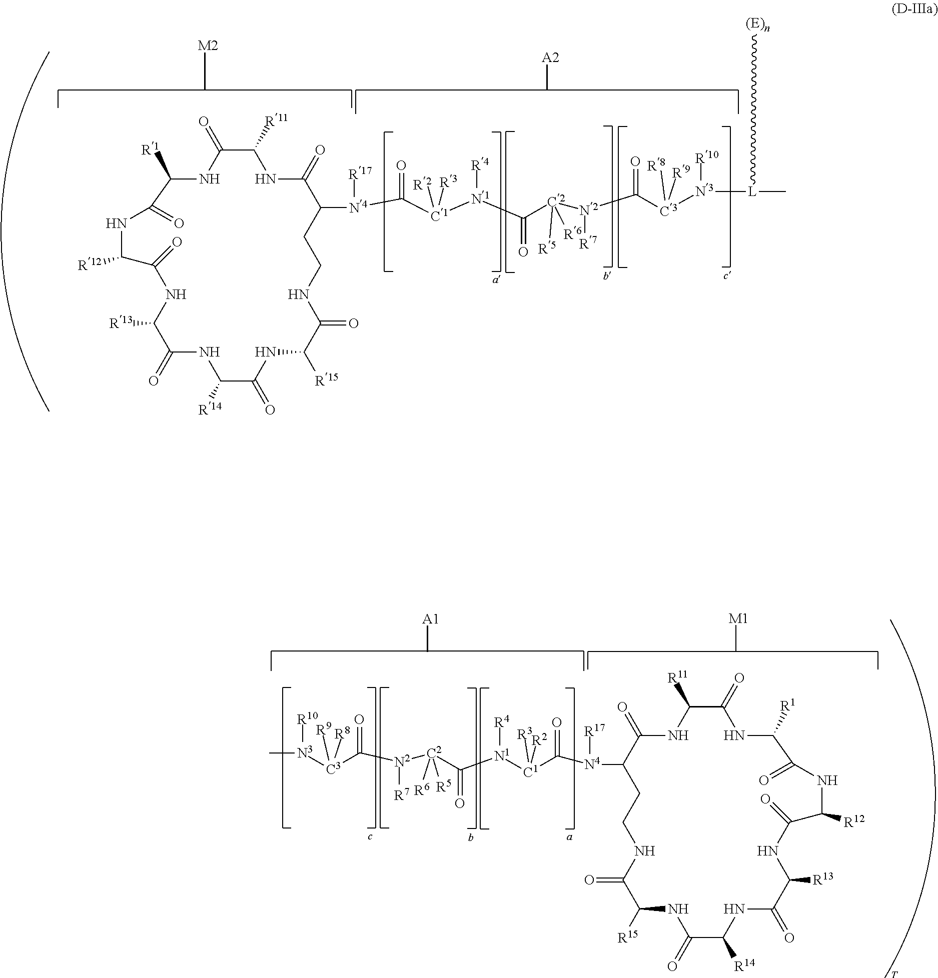

or a pharmaceutically acceptable salt thereof.

In some embodiments, the conjugate is described by formula (D-IIIb):

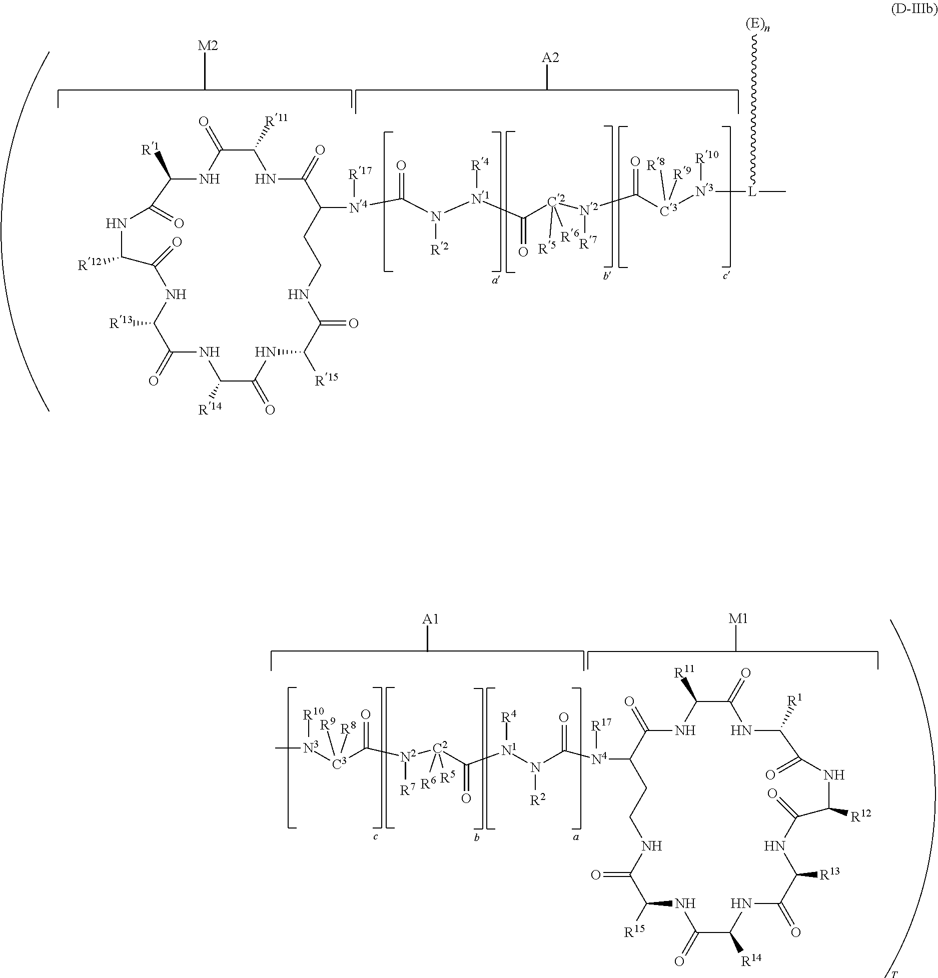

or a pharmaceutically acceptable salt thereof.

In some embodiments, the conjugate is described by formula (D-IIIc):

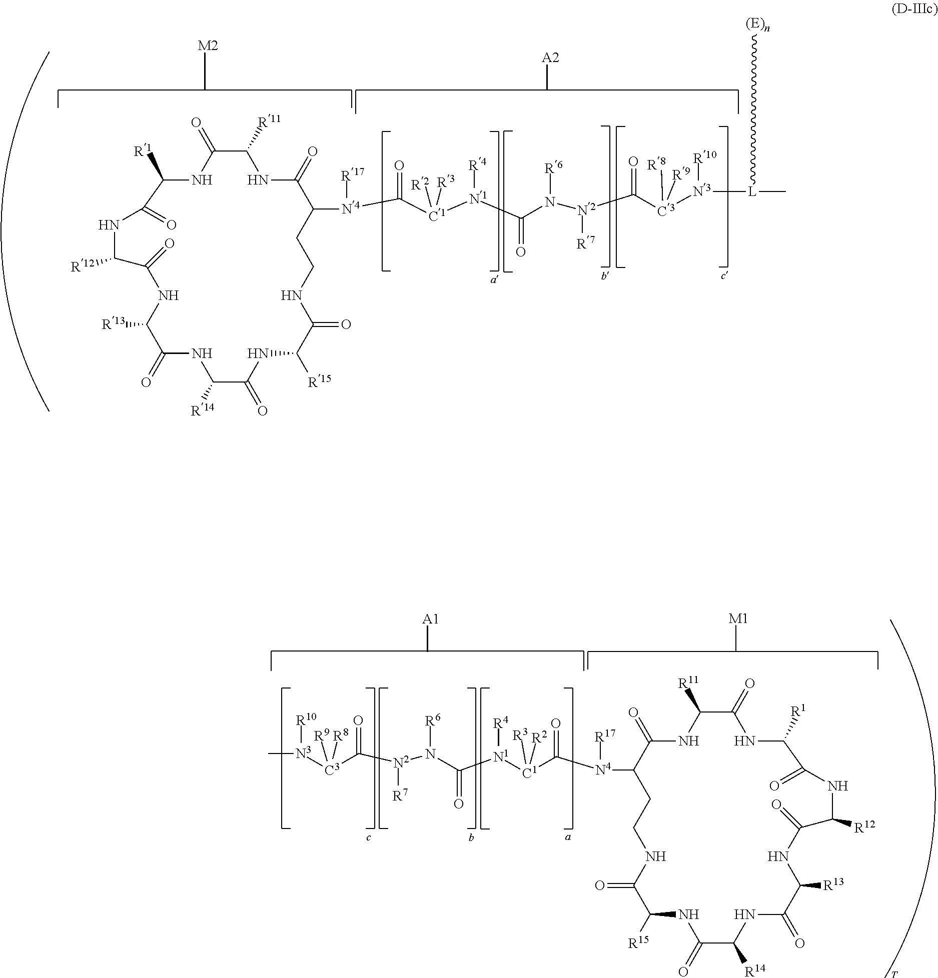

or a pharmaceutically acceptable salt thereof.

In some embodiments, the conjugate is described by formula (D-IIId):

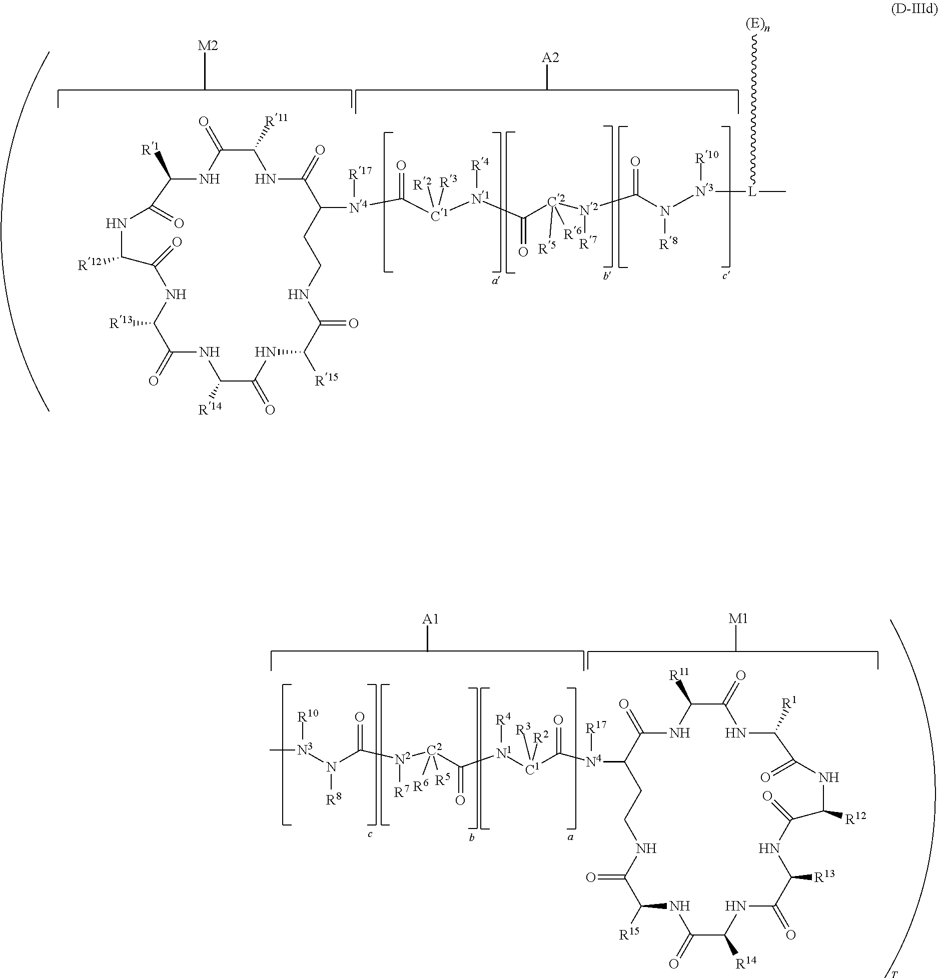

or a pharmaceutically acceptable salt thereof.

In some embodiments, each of R¹, R¹², R′¹, and R′¹² is, independently, a lipophilic moiety; each of R¹¹, R¹³, R¹⁴, R′¹¹, R′¹³, and R′¹⁴ is, independently, optionally substituted C1-C5 alkamino, a polar moiety, or a positively charged moiety; and/or each of R¹⁵ and R′¹⁵ is, independently, a polar moiety.

In some embodiments, each of R¹ and R¹² is a lipophilic moiety. In some embodiments, each of R′¹ and R′¹² is a lipophilic moiety. In some embodiments, each lipophilic moiety is, independently, optionally substituted C1-C20 alkyl, optionally substituted C5-C15 aryl, optionally substituted C6-C35 alkaryl, or optionally C5-C10 substituted heteroaryl. In some embodiments, each lipophilic moiety is, independently, C1-C8 alkyl, methyl substituted C2-C4 alkyl, (C1-C10)alkylene(C6)aryl, phenyl substituted (C1-C10)alkylene(C6)aryl, or alkyl substituted C4-C9 heteroaryl. In some embodiments, each lipophilic moiety is, independently, benzyl, isobutyl, sec-butyl, isopropyl, n-propyl, methyl, biphenylmethyl, n-octyl, or methyl substituted indolyl.

In some embodiments, each of R¹¹, R¹³, R¹⁴, R′¹¹, R′¹³, and R′¹⁴ is independently optionally substituted C1-C5 alkamino (e.g., CH₂CH₂NH₂).

In some embodiments, each of R¹⁵ and R′¹⁵ is a polar moiety. In some embodiments, each polar moiety includes a hydroxyl group, a carboxylic acid group, an ester group, or an amide group. In some embodiments, each polar moiety is hydroxyl substituted C1-C4 alkyl. In some embodiments, each polar moiety is CHCH₃OH.

In some embodiments, each of R¹⁷ and R′¹⁷ is H. In some embodiments, each of R¹⁷ and R′¹⁷ is methyl.

In some embodiments of this aspect, the conjugate is described by formula (D-IVa):

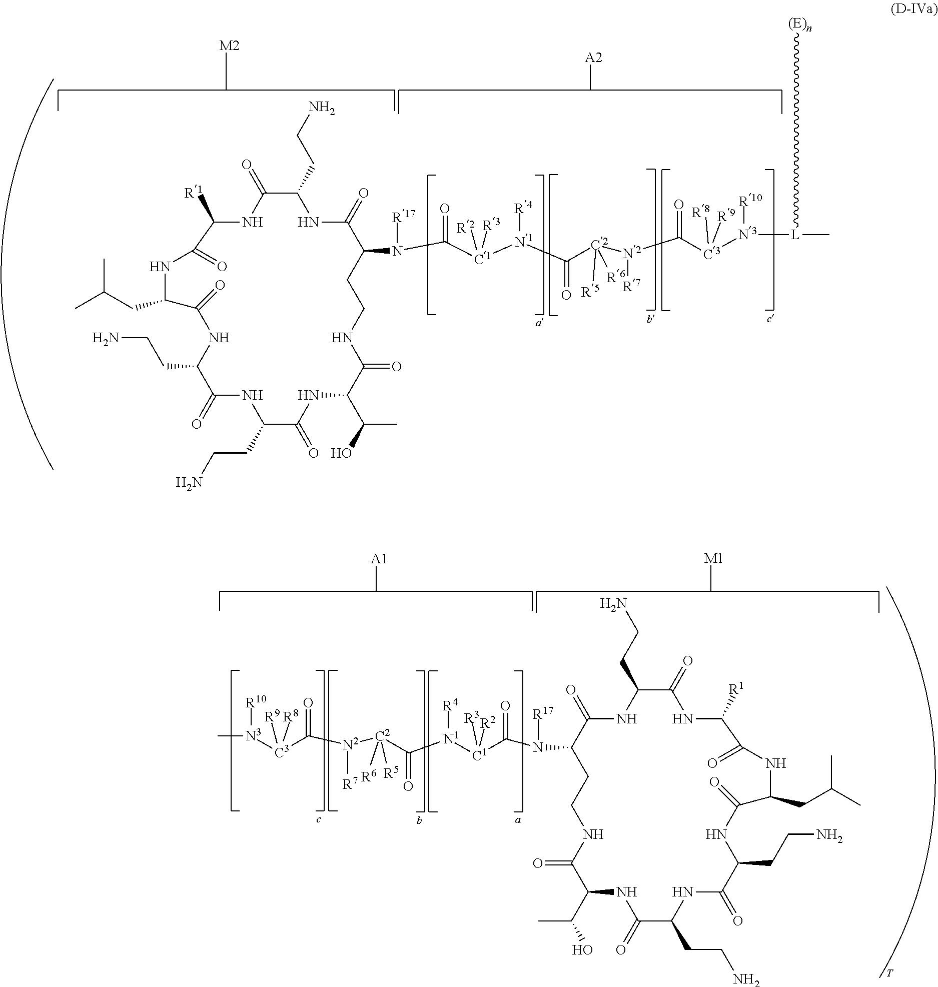

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In some embodiments, the conjugate is described by formula (D-IVa-1):

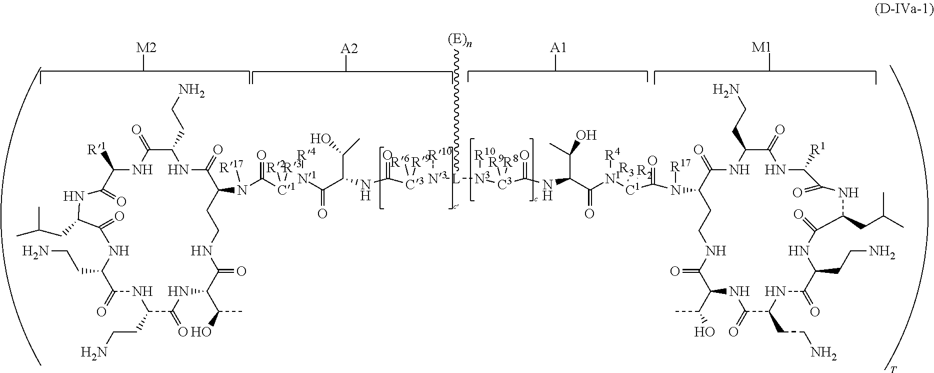

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In some embodiments, the conjugate is described by formula (D-IVa-2):

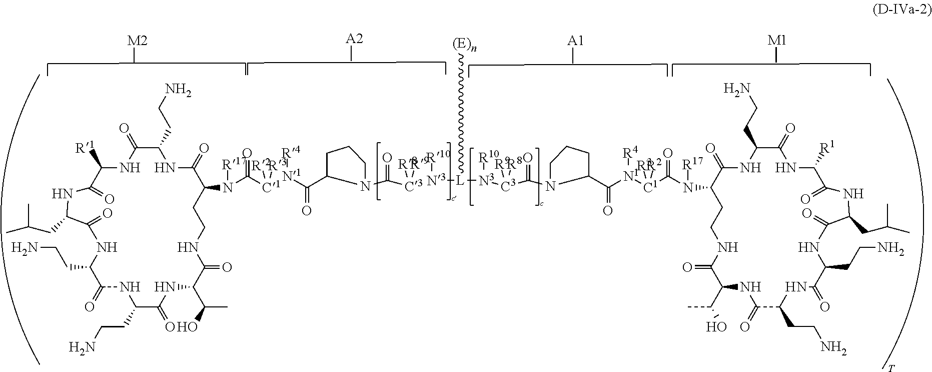

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In some embodiments, the conjugate is described by formula (D-IVa-2a):

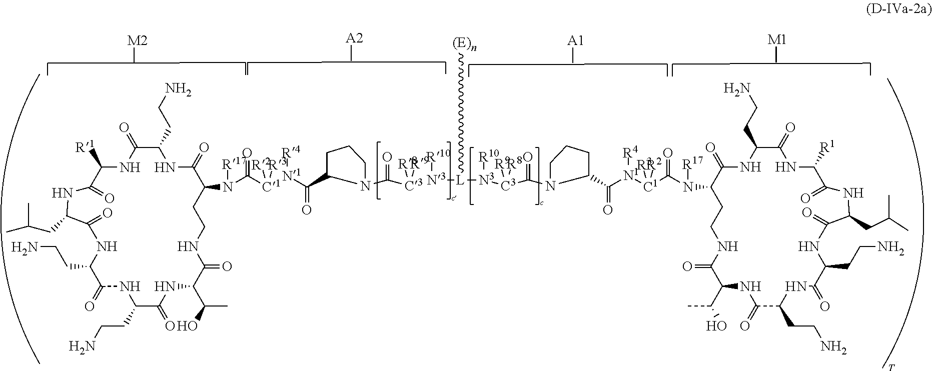

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In some embodiments, the conjugate is described by formula (DIV-2)

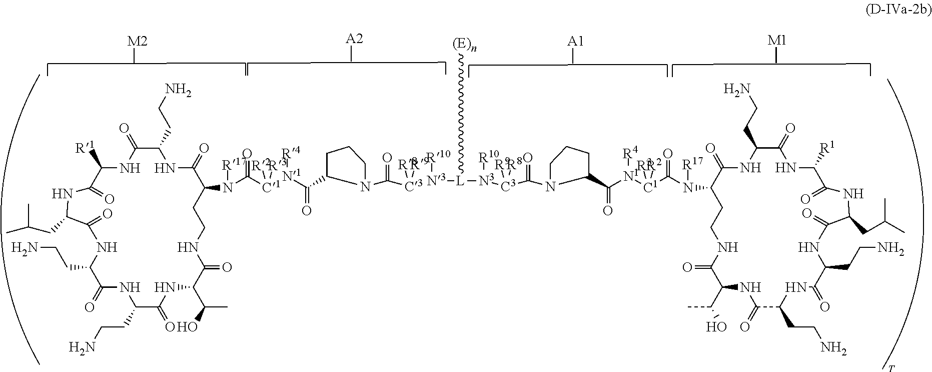

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In some embodiments, the conjugate is described by formula (D-IVa-3):

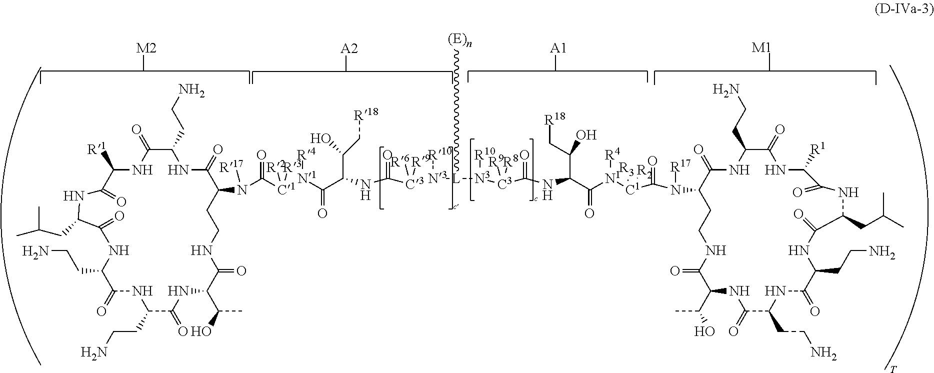

wherein each of R¹ and R′¹ is, independently, optionally substituted benzyl. CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R′¹⁶ and R¹⁶ is, independently, a lipophilic moiety, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments, each of R′¹⁶ and R¹⁶ is, independently, C1-C6 alkyl or benzyl.

In some embodiments, the conjugate is described by formula (D-IVb):

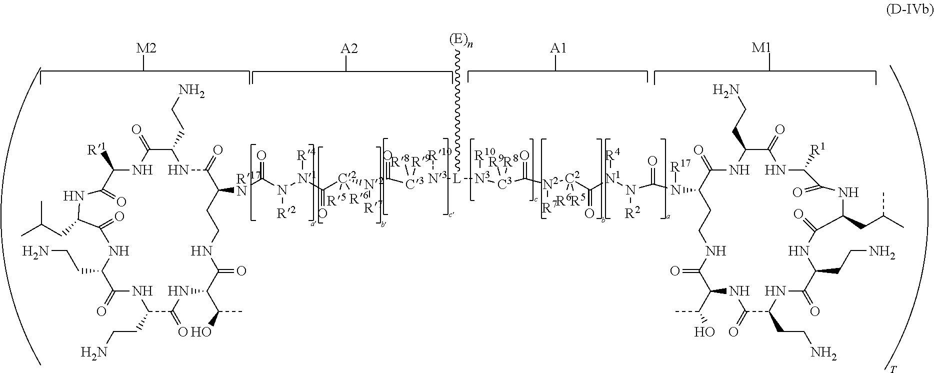

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In some embodiments, the conjugate is described by formula (D-IVb-1):

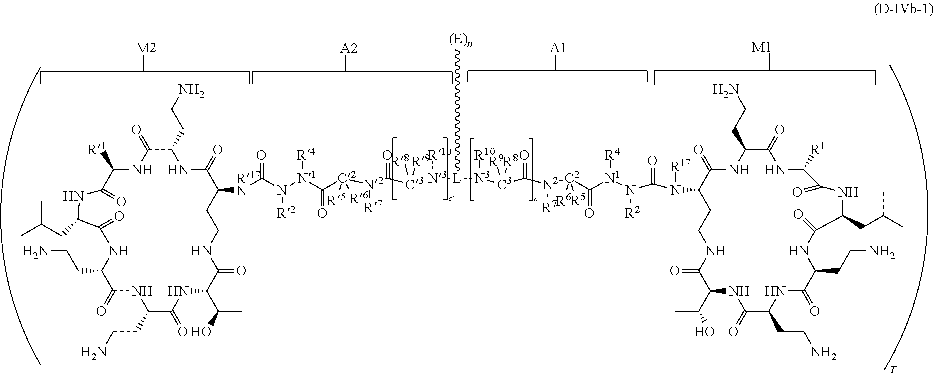

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In some embodiments, each of R² and R′² is, independently, H, C1-C8 alkyl, or C2-C8 alkamino.

In some embodiments, the conjugate is described by formula (D-IVc):

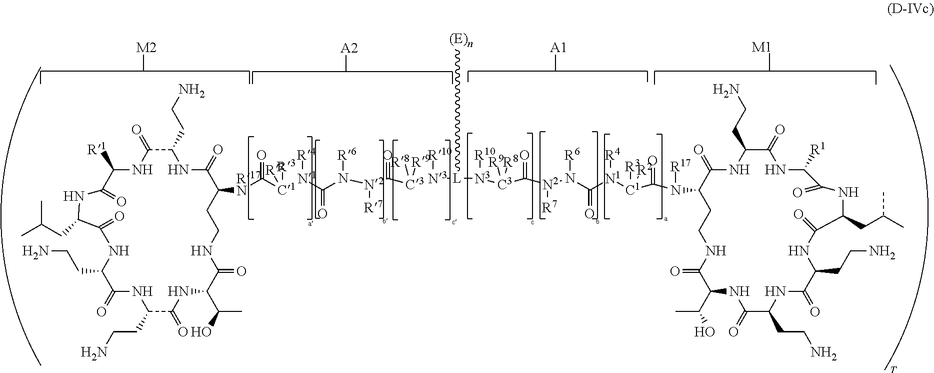

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In some embodiments, the conjugate is described by formula (D-IVc-1):

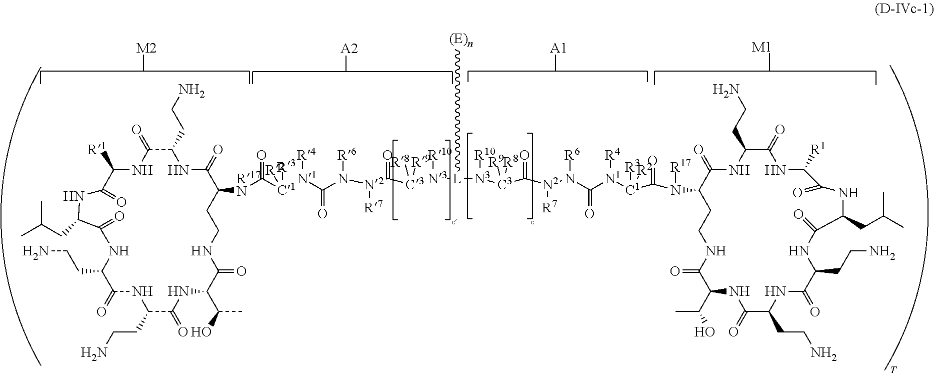

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In some embodiments, each of R⁶ and R′⁶ is, independently, H, C1-C8 alkyl, or C2-C8 alkamino.

In some embodiments, the conjugate is described by formula (D-IVd):

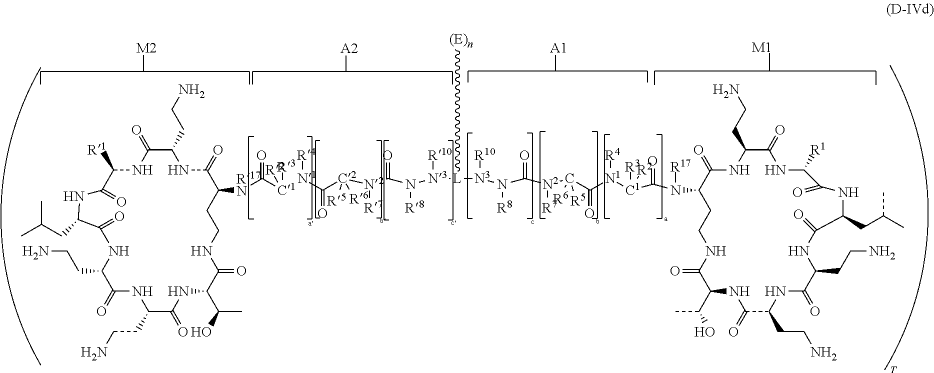

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In some embodiments, each of R⁸ and R′⁸ is, independently, H, C1-C8 alkyl, or C2-C8 alkamino. In some embodiments, each of R¹⁷ and R′¹⁷ is H. In some embodiments, each of R¹⁷ and R′⁷ is methyl.

In some embodiments, the conjugate is described by formula (D-V):

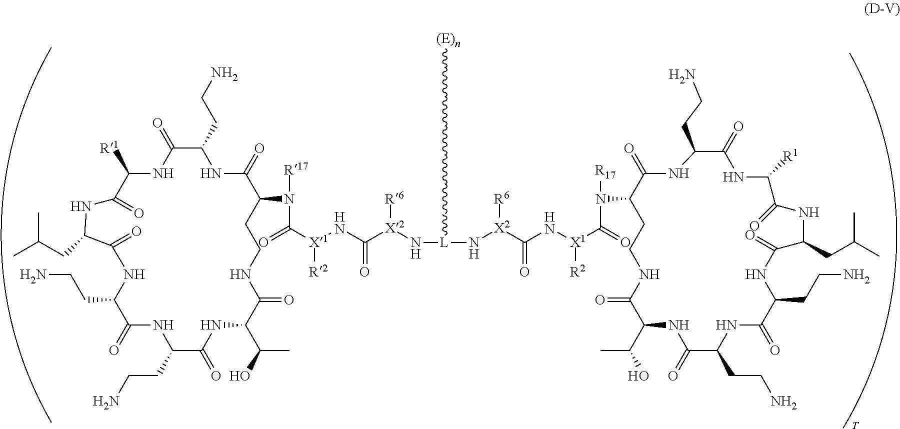

wherein each of R¹ and R′¹ is, independently, optionally substituted benzyl. CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R², R⁸, R′², and R′⁸ is, independently, H, a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments, the conjugate is described by formula (D-Va):

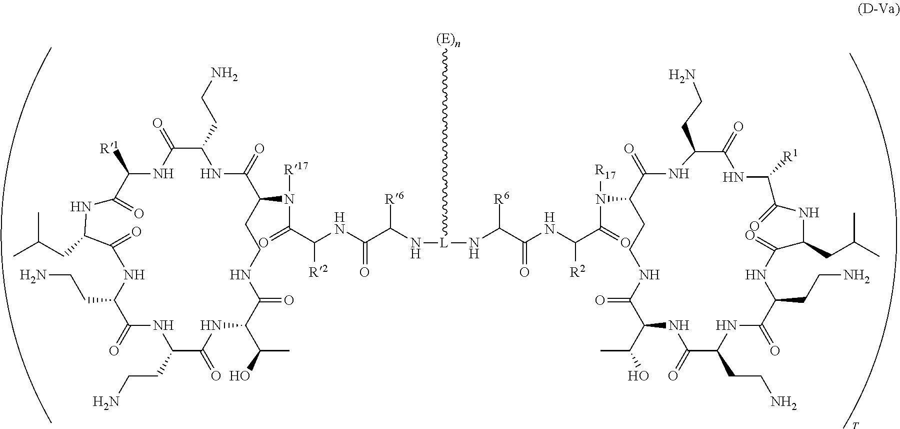

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; and each of R², R⁶, R′², and R′⁶ is, independently. H, a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments, the conjugate is described by formula (D-Vb):

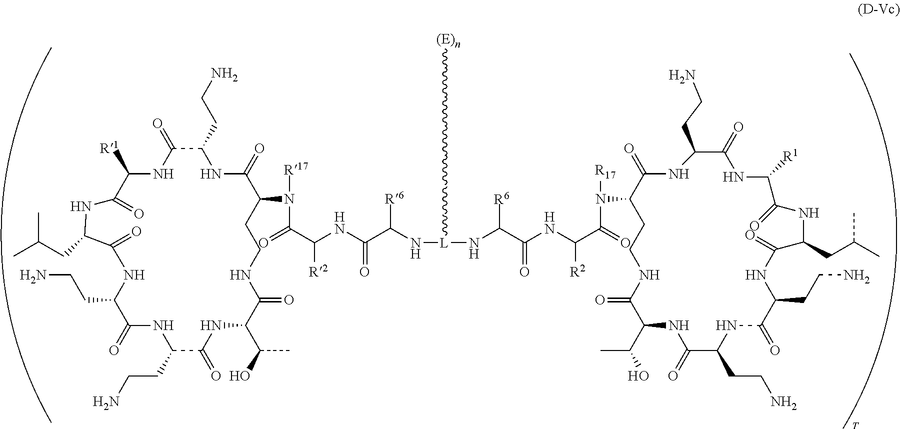

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; and each of R², R⁶, R′², and R′⁶ is, independently, H, a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments, the conjugate is described by formula (D-Vc):

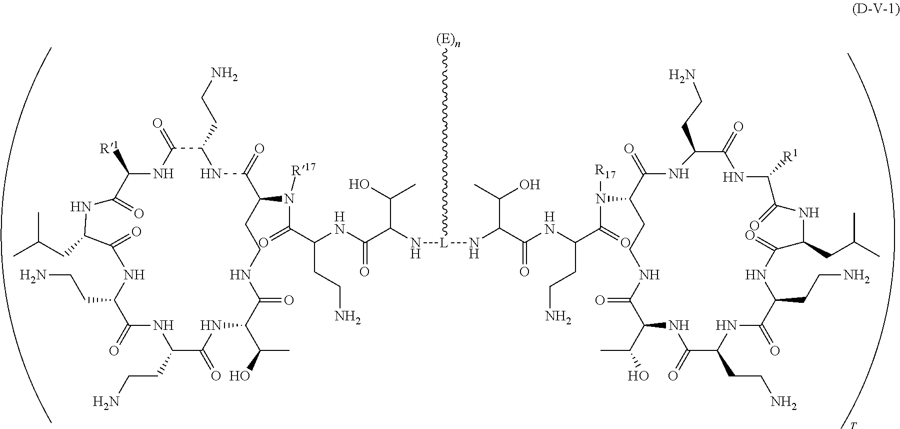

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; and each of R², R⁶, R′², and R′⁶ is, independently, H, a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments, the conjugate is described by formula (D-V-1):

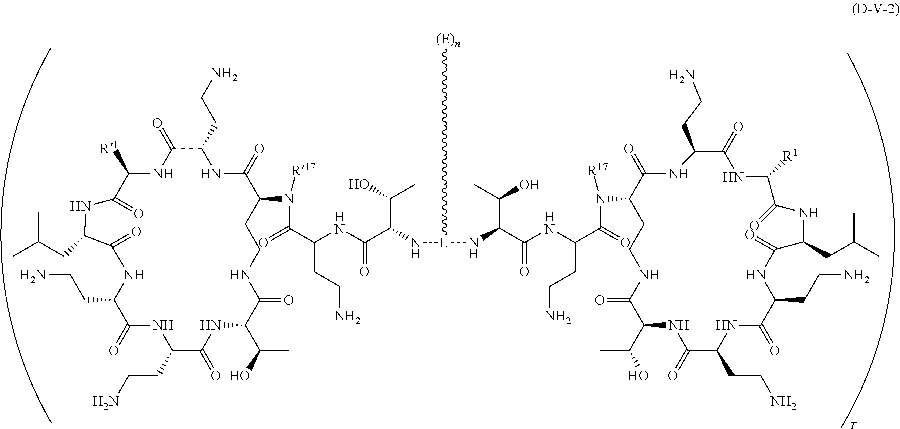

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In some embodiments, the conjugate is described by formula (D-V-2):

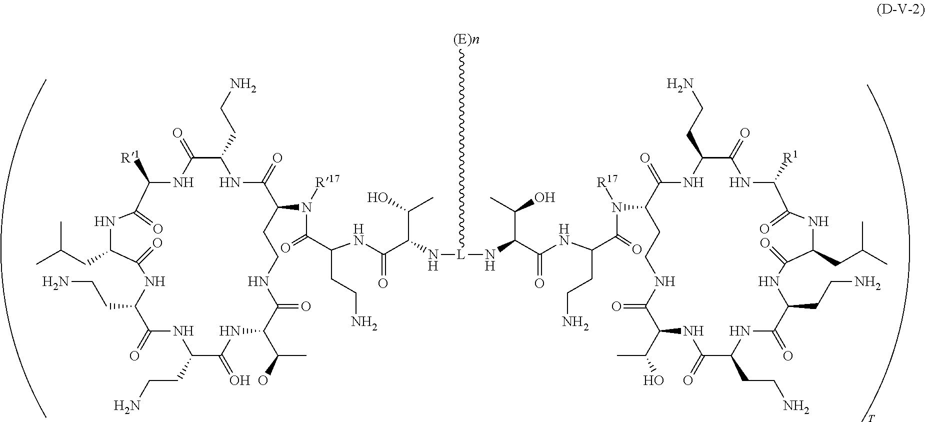

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In some embodiments, the conjugate is described by formula (D-V-3):

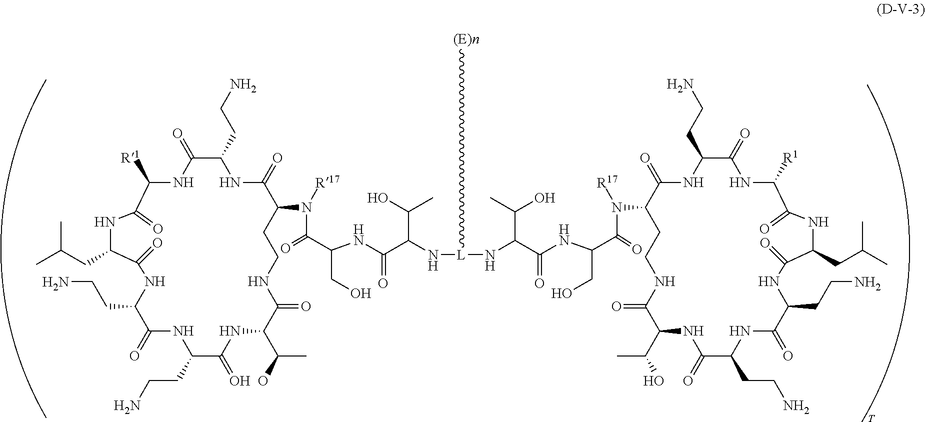

wherein each of R¹ and R′¹ is (independently, optionally substituted benzyl, CH₂CH(CH₃)₂ cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In some embodiments, the conjugate is described by formula (D-V-4):

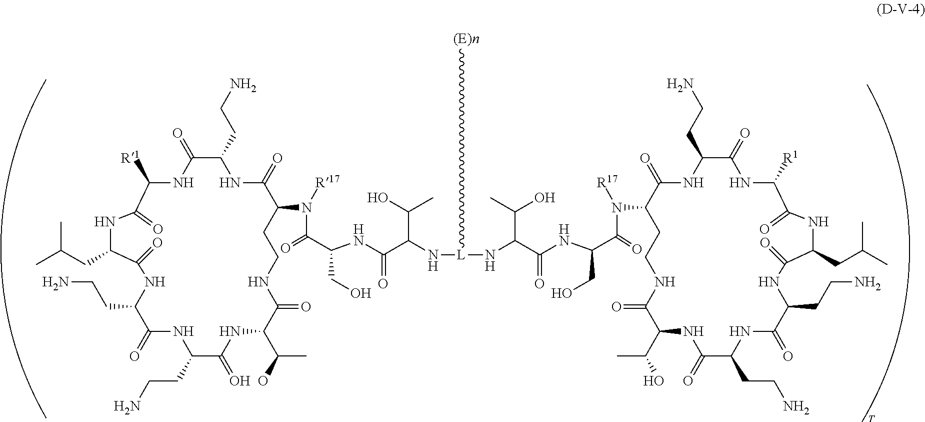

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In some embodiments, the conjugate is described by formula (D-V-5):

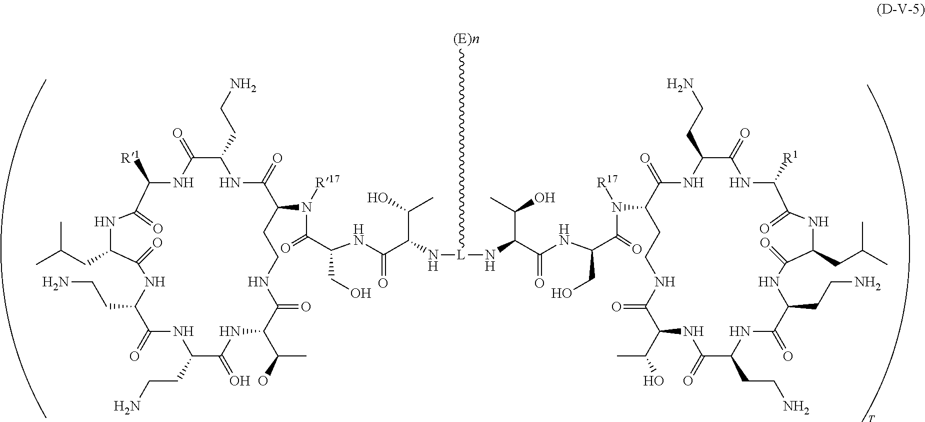

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In some embodiments, the conjugate is described by formula (D-V-6):

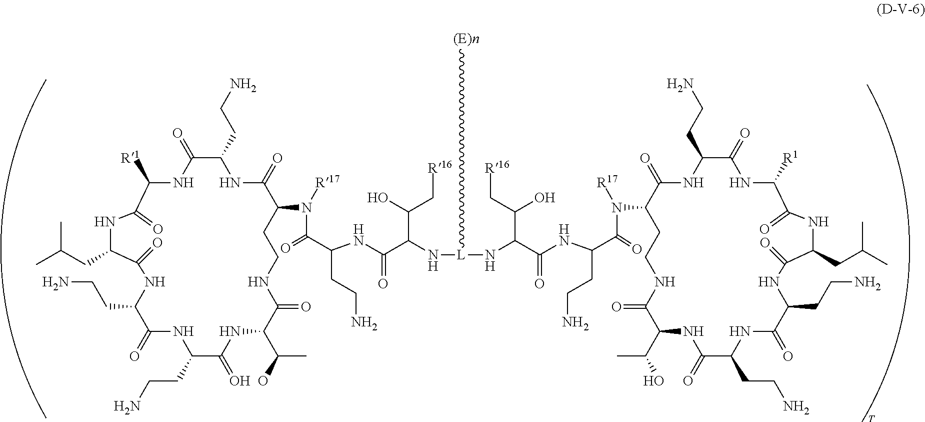

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R′¹⁶ and R¹⁶ is, independently, a lipophilic moiety, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments, the conjugate is described by formula (D-V-7a):

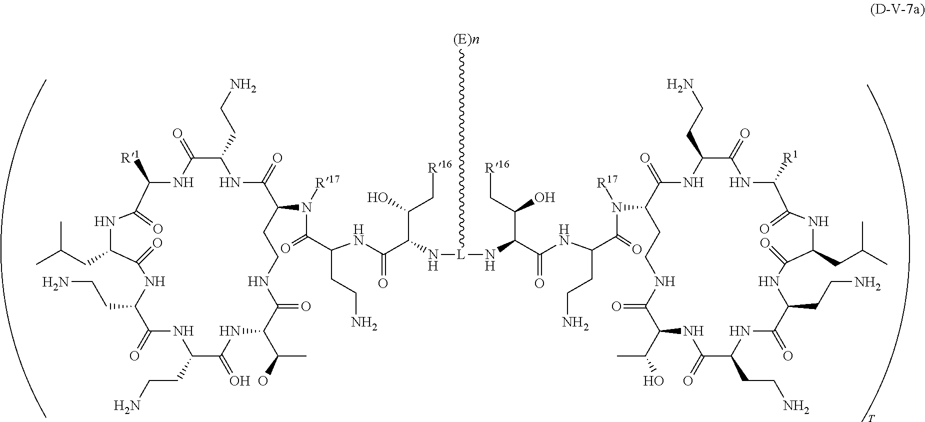

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R′¹⁶ and R¹⁶ is, independently, a lipophilic moiety, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments, the conjugate is described by formula (D-V-7a-1):

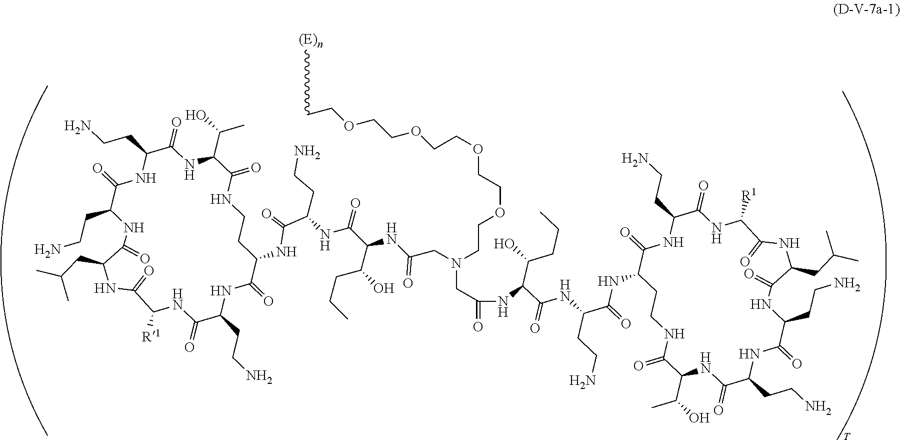

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In some embodiments, the conjugate is described by formula (D-V-7a-2):

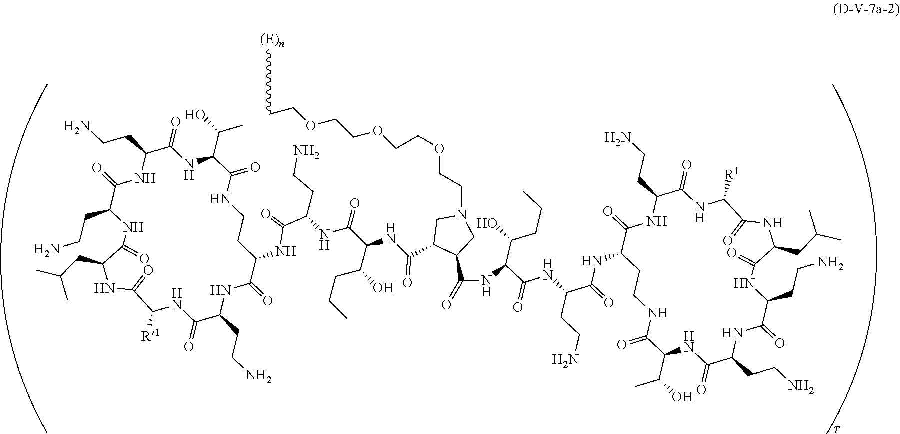

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-V-7a-3):

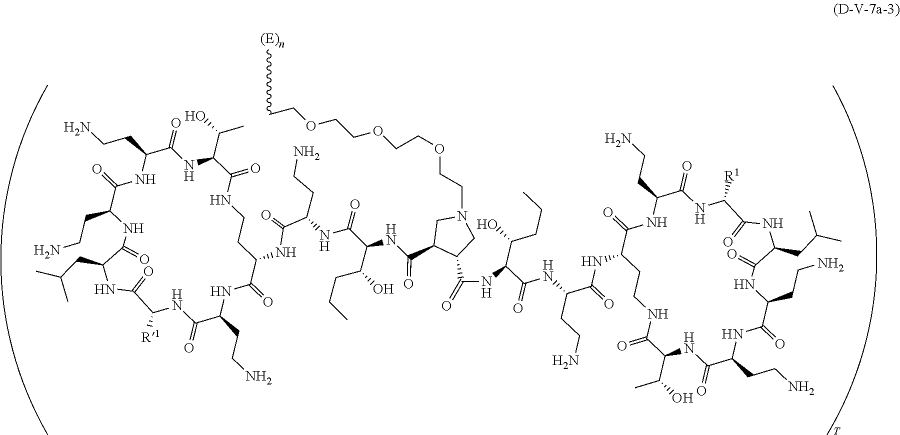

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-V-7b):

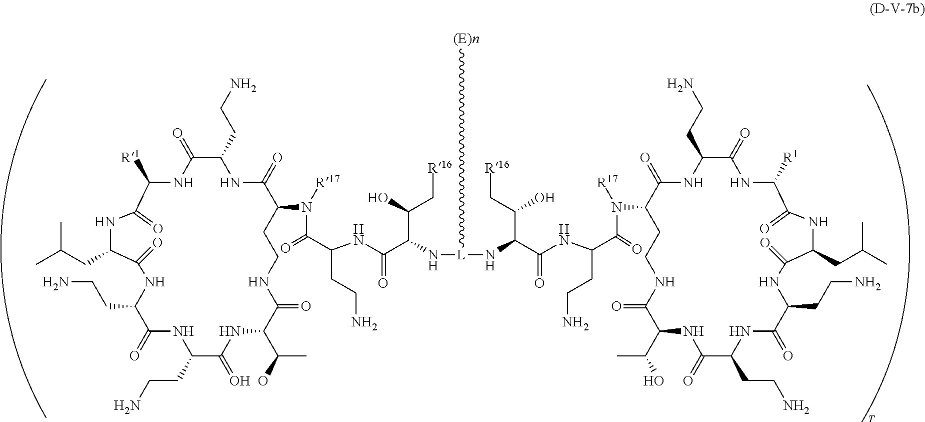

wherein each of R¹ and R′¹ is, independently, optionally substituted benzyl, CH₂CH(CH₃)₂, cyclopropylmethyl, cyclopentylmethyl, cyclohexylmethyl, napthylmethyl, optionally substituted thiophenylmethyl, or optionally substituted furanylmethyl; and each of R′¹⁶ and R¹⁶ is, independently, a lipophilic moiety, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; or a pharmaceutically acceptable salt thereof.

In some embodiments, the conjugate is described by formula (D-V-8):

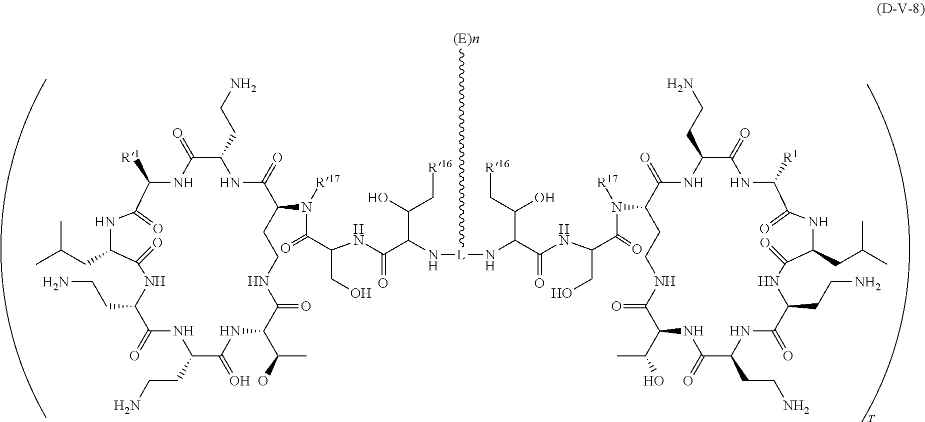

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R′¹⁶ and R¹⁶ is, independently, a lipophilic moiety, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments, the conjugate is described by formula (D-V-9):

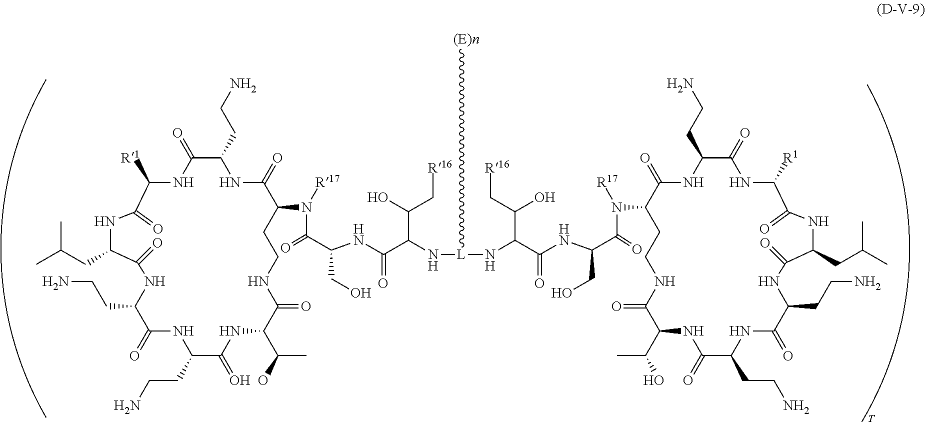

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R′¹⁶ and R¹⁶ is, independently, a lipophilic moiety, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments, the conjugate is described by formula (D-V-10):

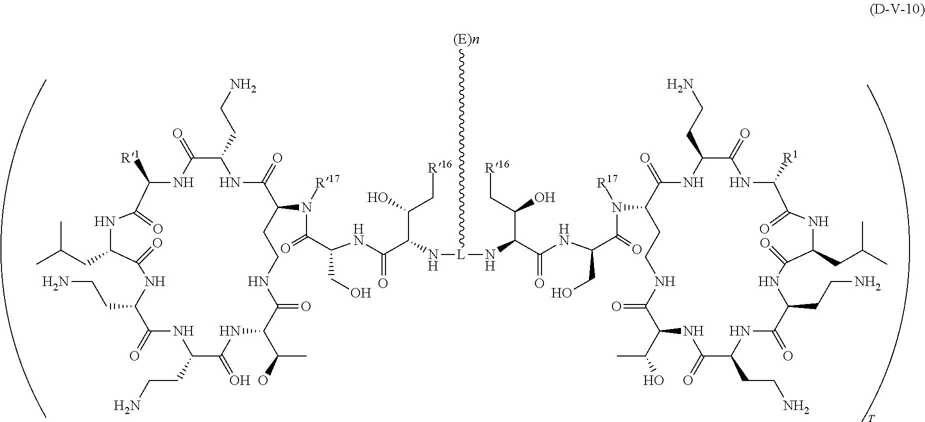

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R′¹⁶ and R¹⁶ is, independently, a lipophilic moiety, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments, each of R′¹⁶ and R¹⁶ is, independently, C1-C6 alkyl or benzyl.

In some embodiments, the conjugate is described by formula (D-V-11):

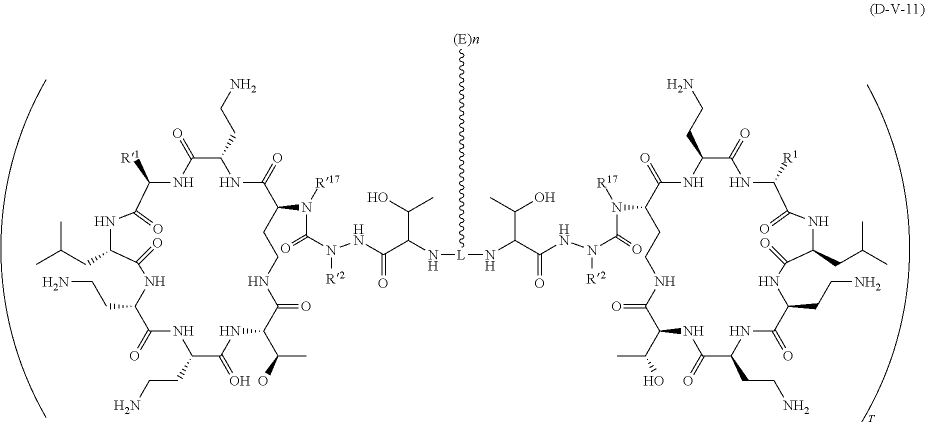

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; and each of R′² and R² is, independently, H, C1-C8 alkyl, or C2-C8 alkamino; or a pharmaceutically acceptable salt thereof.

In some embodiments, the conjugate is described by formula (D-V-12):

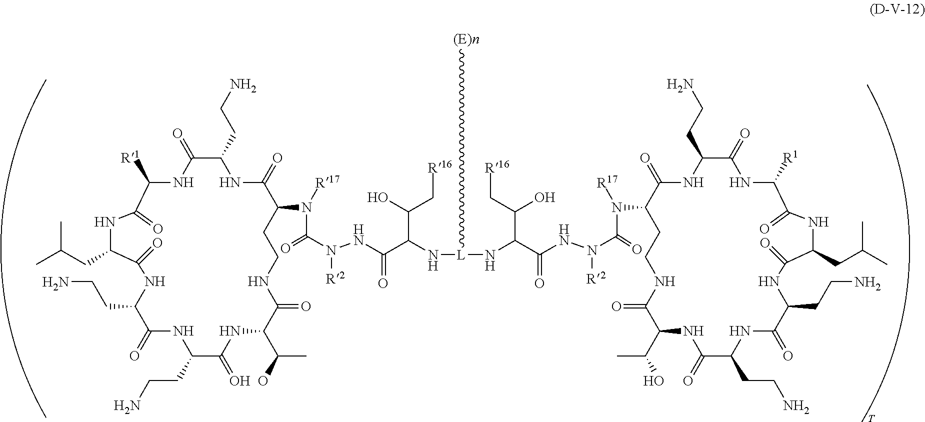

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R′² and R² is, independently, H, C1-C8 alkyl, or C2-C8 alkamino; and each of R′¹⁶ and R¹⁶ is, independently, a lipophilic moiety, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; or a pharmaceutically acceptable salt thereof.

In some embodiments, each of R′¹⁶ and R¹⁶ is, independently, C1-C6 alkyl, benzyl, or phenethyl.

In some embodiments, the conjugate is described by formula (D-V-13):

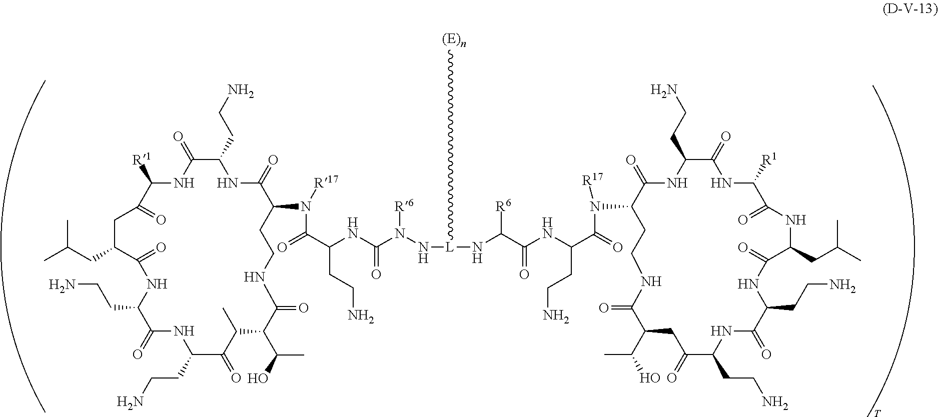

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; and each of R′⁶ and R⁶ is, independently, H, C1-C8 alkyl, or C2-C8 alkamino; or a pharmaceutically acceptable salt thereof.

In some embodiments, the conjugate is described by formula (D-V-14):

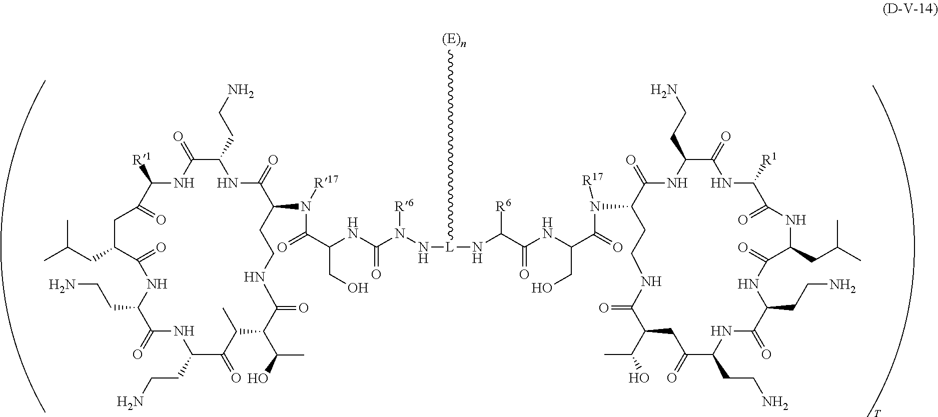

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; and each of R′⁶ and R⁶ is, independently, H, C1-C8 alkyl, or C2-C8 alkamino; or a pharmaceutically acceptable salt thereof.

In some embodiments of this aspect, the conjugate is described by formula (D-V):

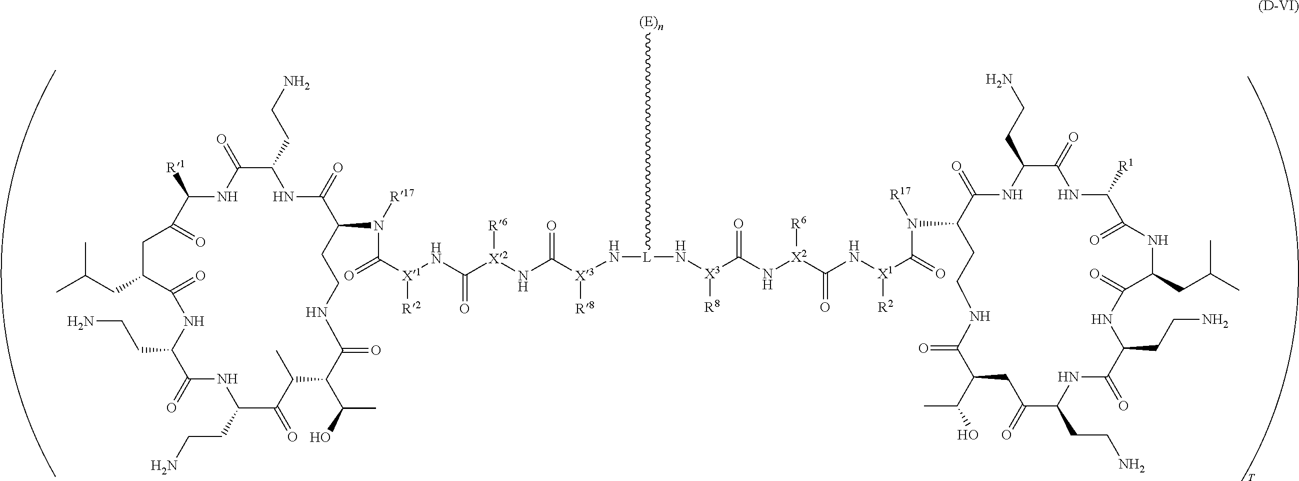

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R², R⁶, R⁸, R′², R′⁶, and R′⁸ is, independently, H, a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments, the conjugate is described by formula (D-VIa):

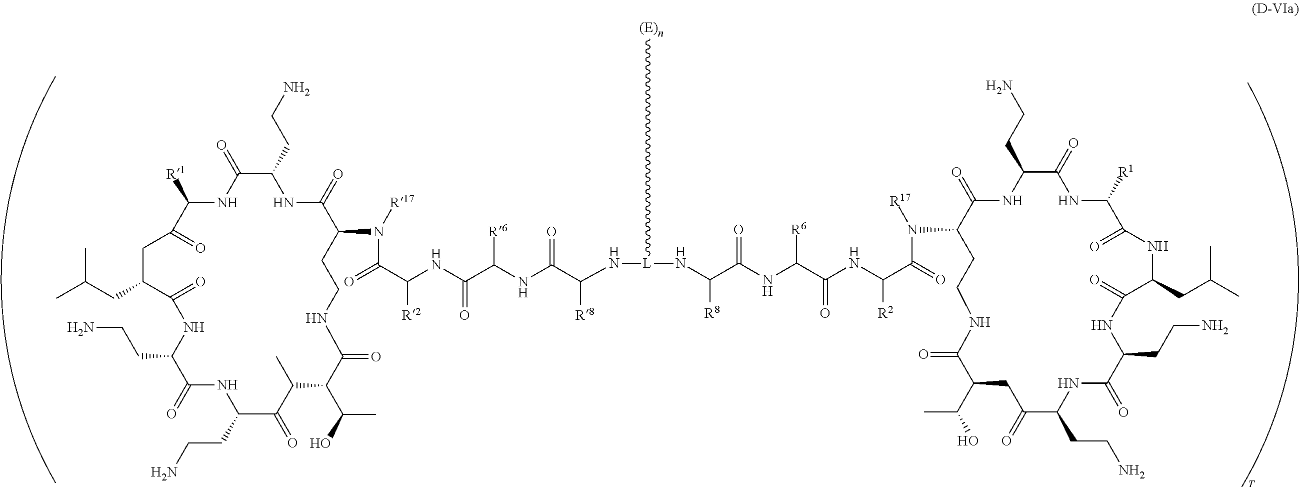

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; and each of R², R⁶, R⁸, R′², R′⁶, and R′⁸ is, independently. H, a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments, the conjugate is described by formula (D-VIb):

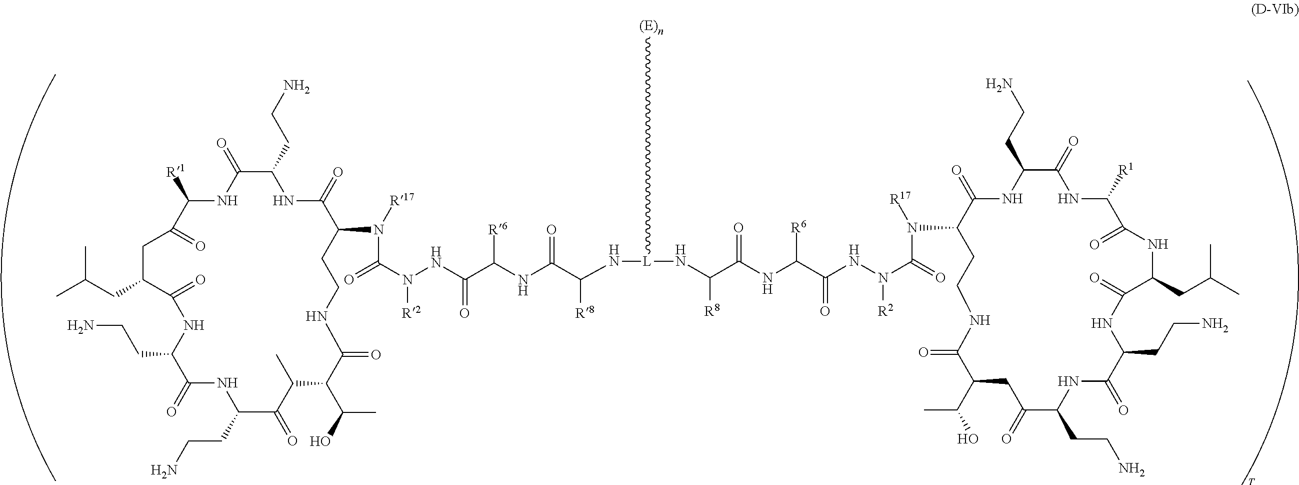

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; and each of R², R⁶, R⁸, R′², R′⁶, and R′⁸ is, independently, H, a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments the conjugate is described by formula (D-VIc):

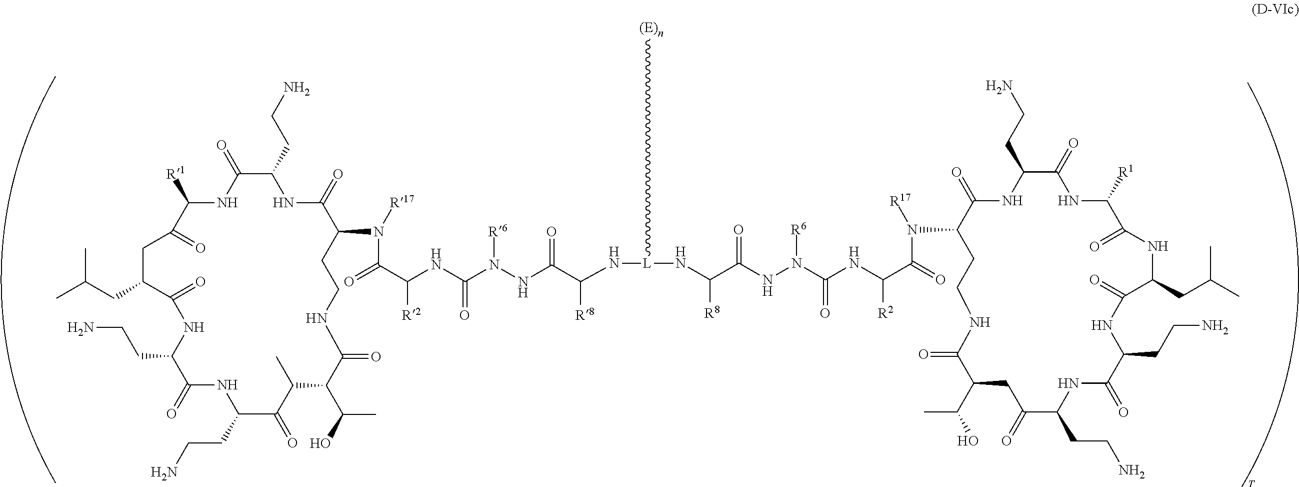

wherein each of R¹ and R′¹ is, independently, optionally substituted benzyl. CH₂CH(CH₃)₂, cyclopropylmethyl, cyclopentylmethyl, cyclohexylmethyl, naphthylmethyl, optionally substituted thiophenylmethyl, or optionally substituted furanylmethyl; and each of R², R⁶, R⁸, R¹², R′⁶, and R′⁸ is, independently, H, a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments, the conjugate is described by formula (D-Vid):

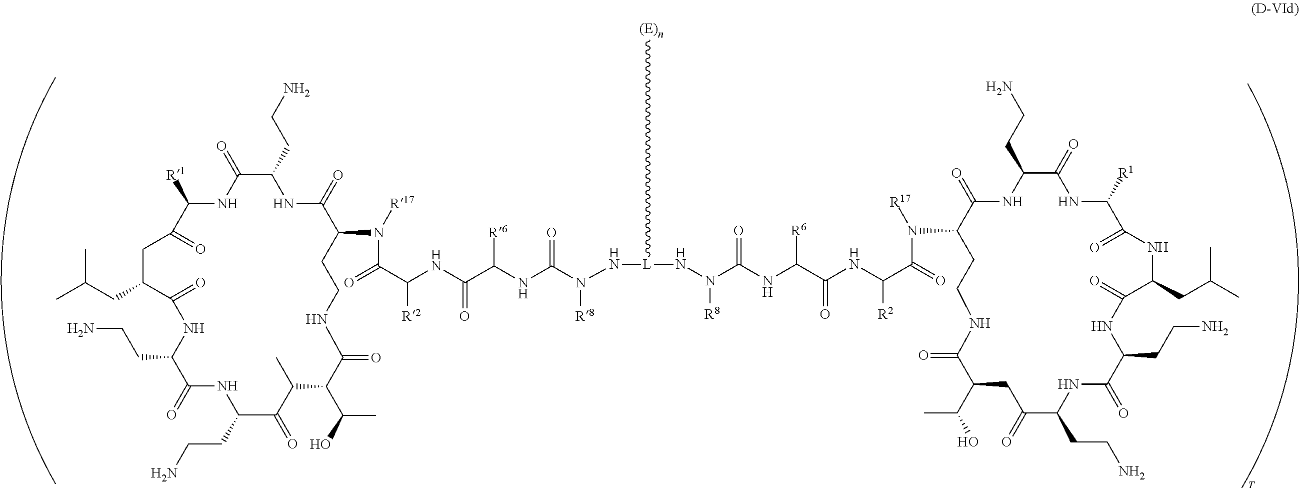

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; and each of R², R⁶, R⁸, R′², R′⁶, and R′⁸ is, independently, H, a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments, the conjugate is described by formula (D-VI-1):

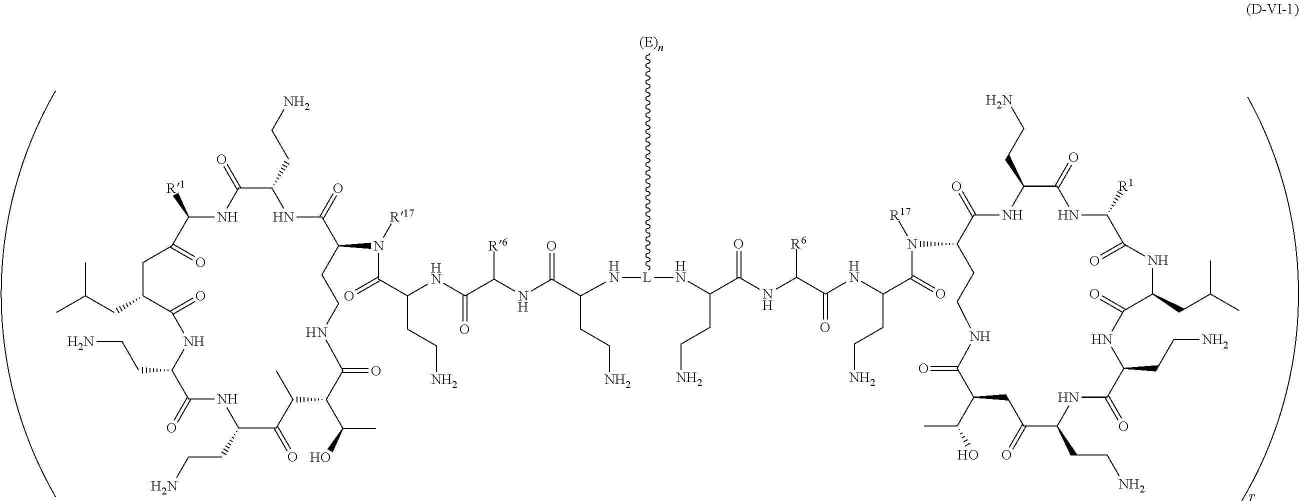

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by the formula (D-V1-1a):

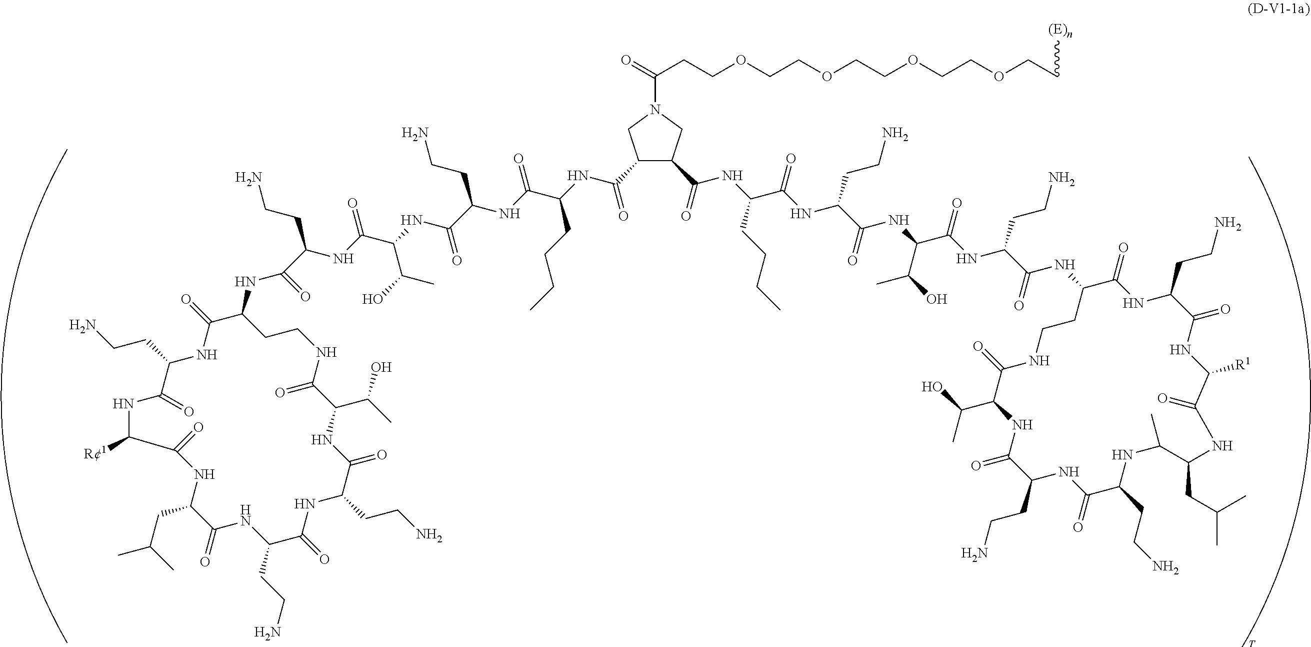

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-2):

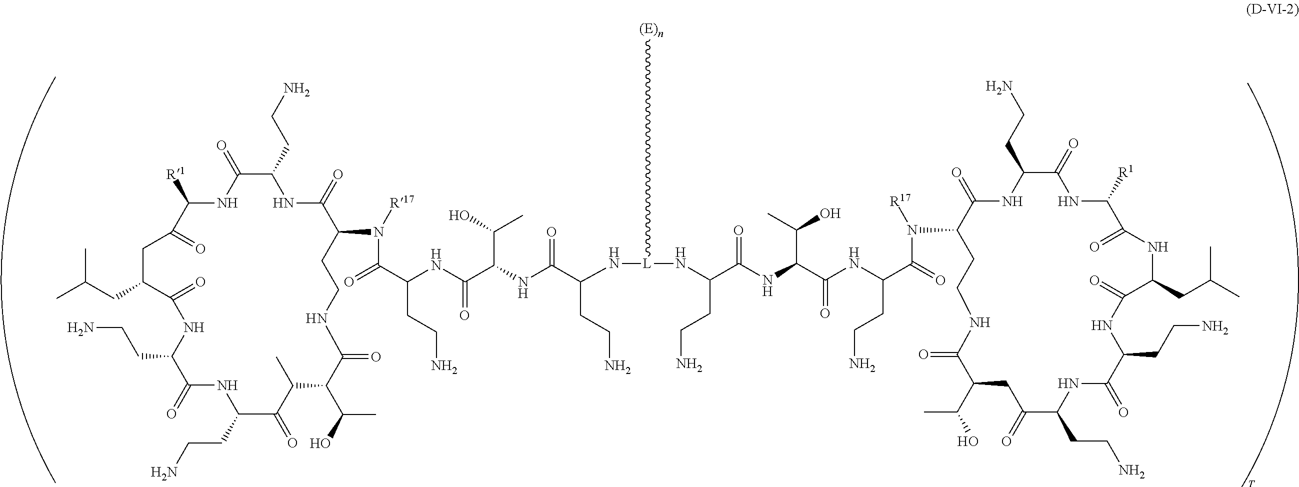

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-2a):

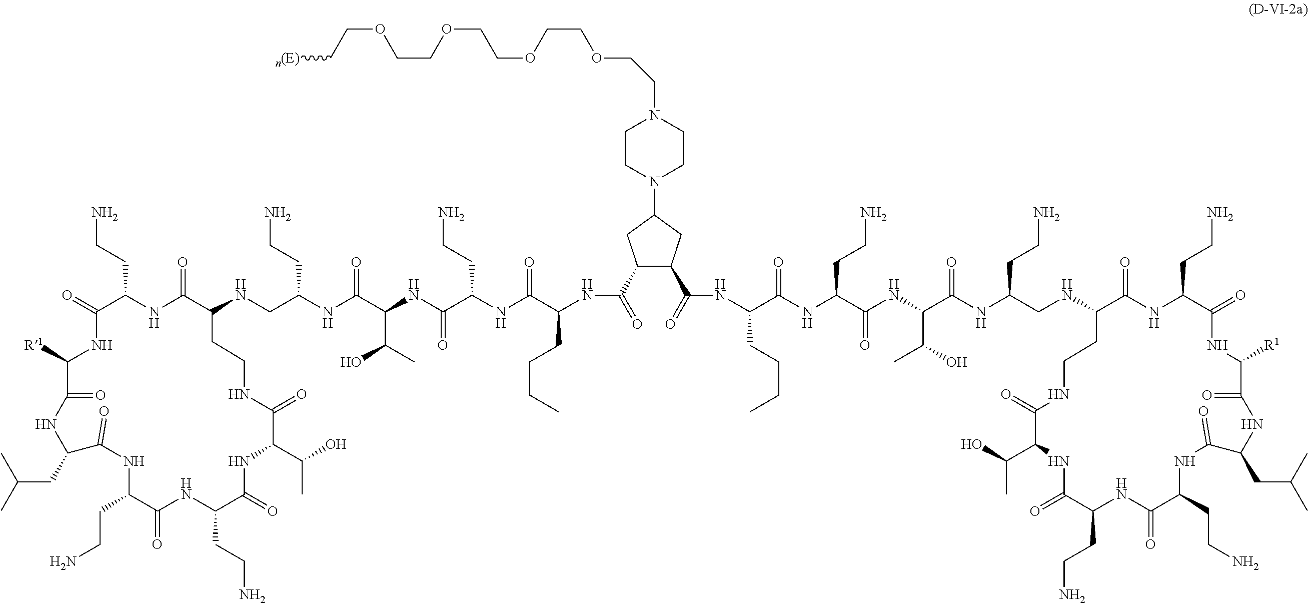

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-2b):

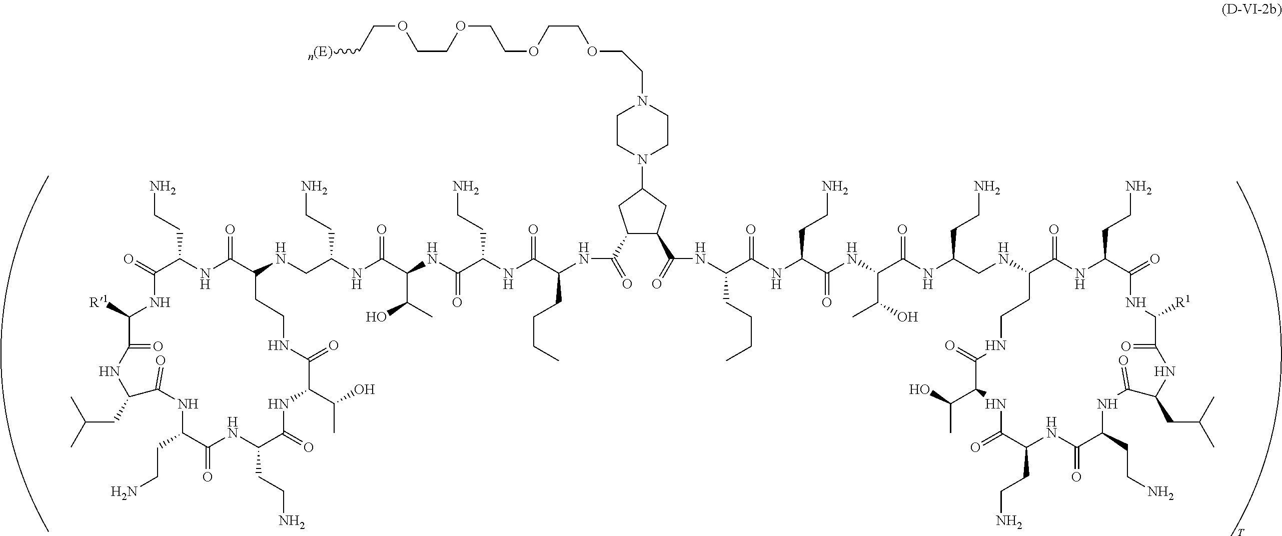

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-2c):

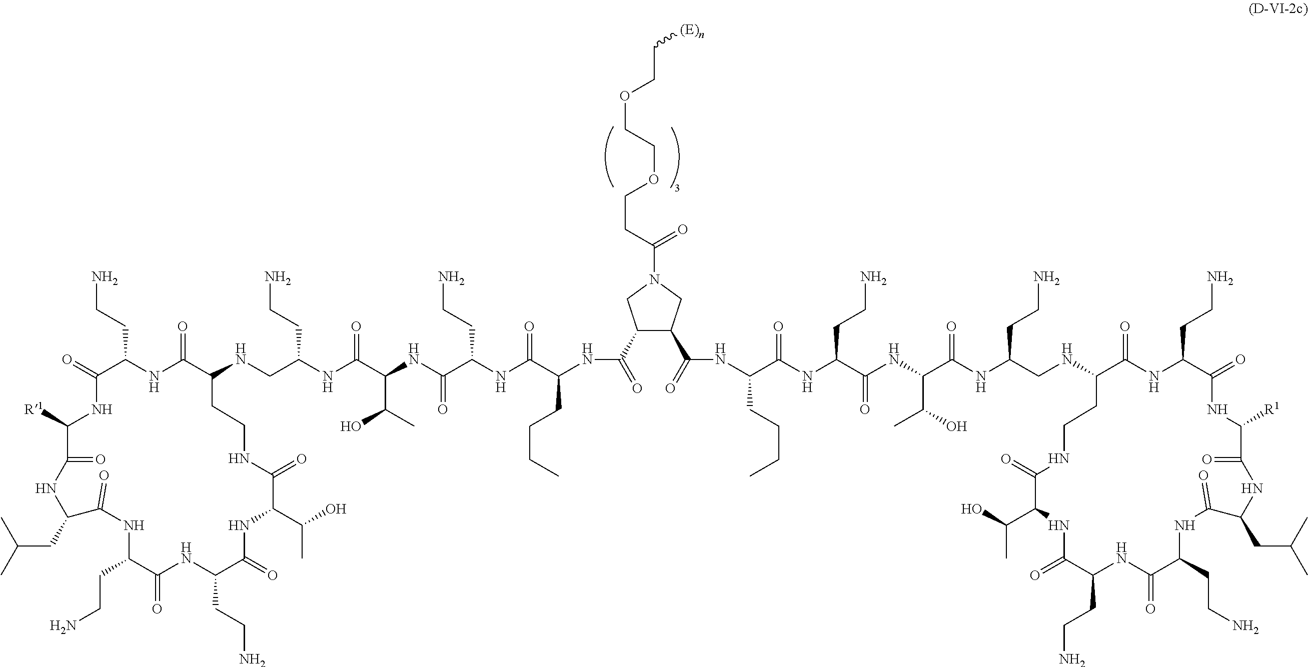

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-2d):

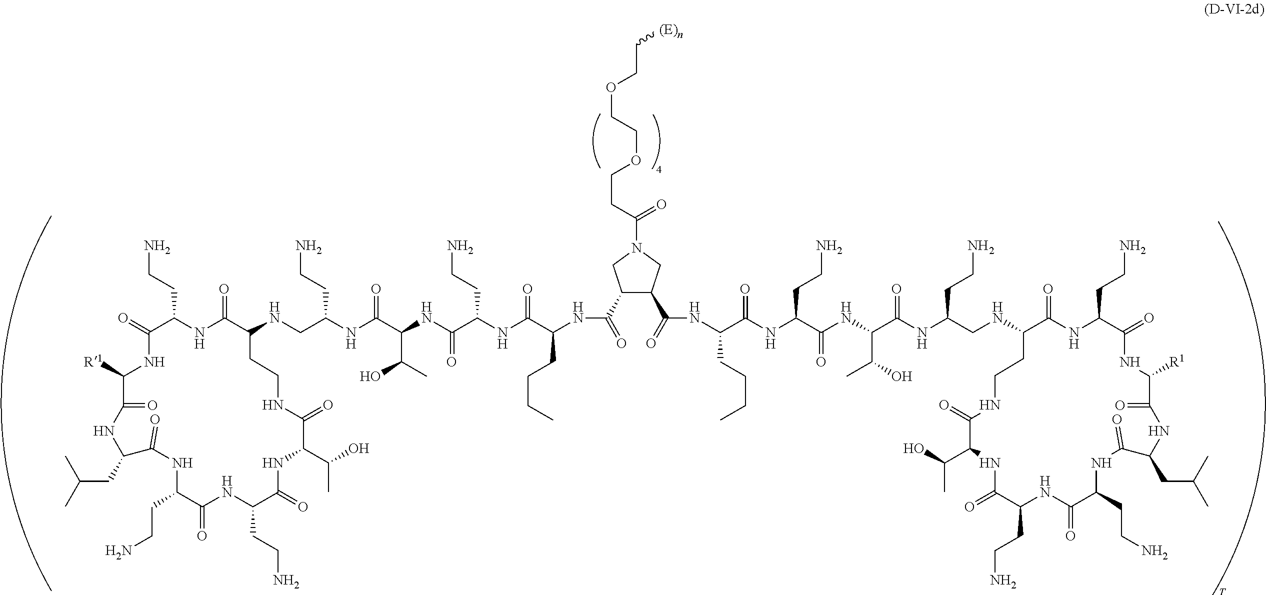

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-2e):

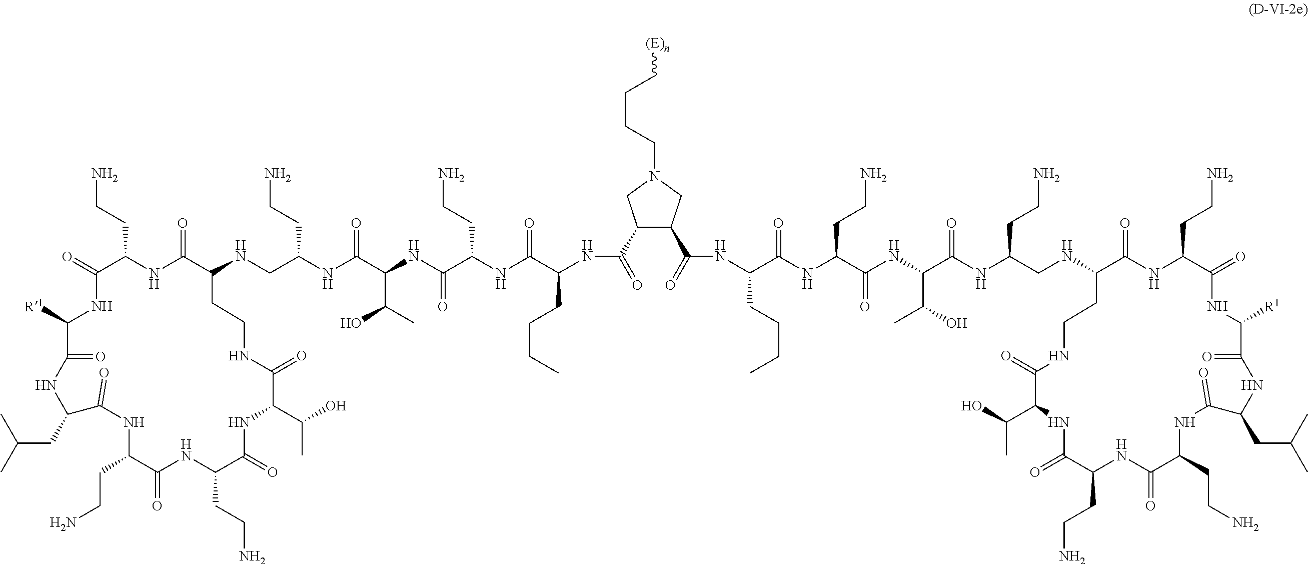

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-2f):

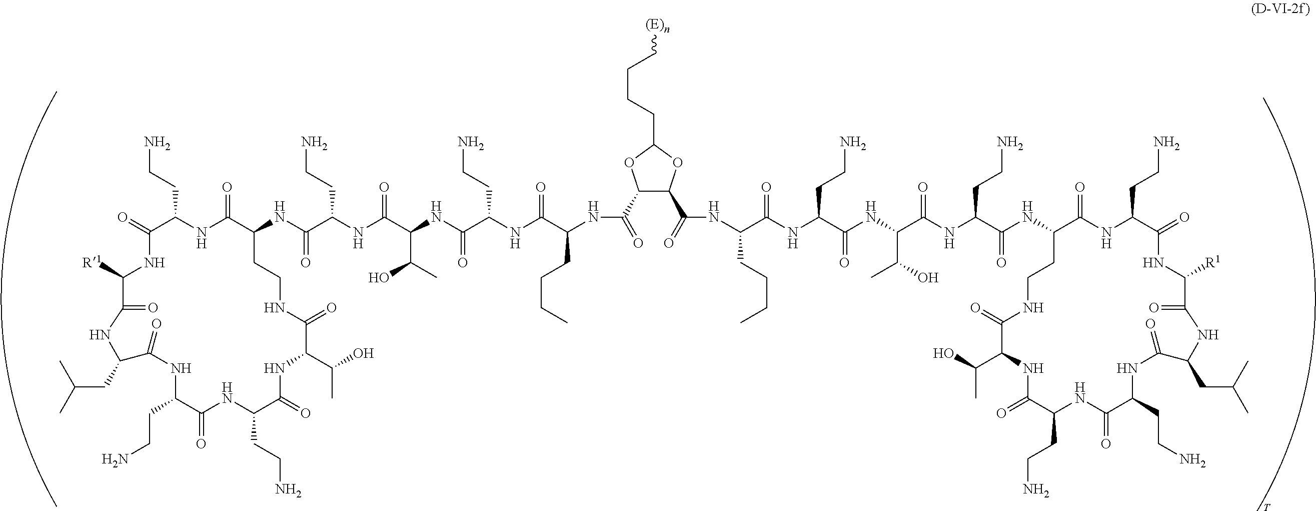

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-2g):

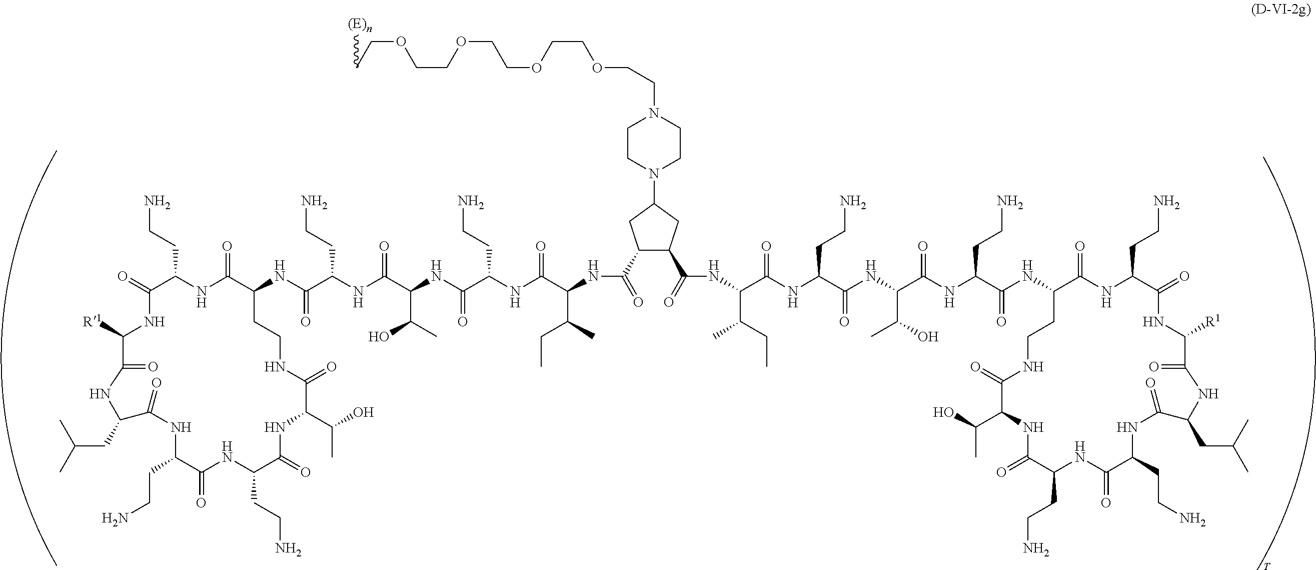

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments the conjugate is described by formula (D-VI-2h):

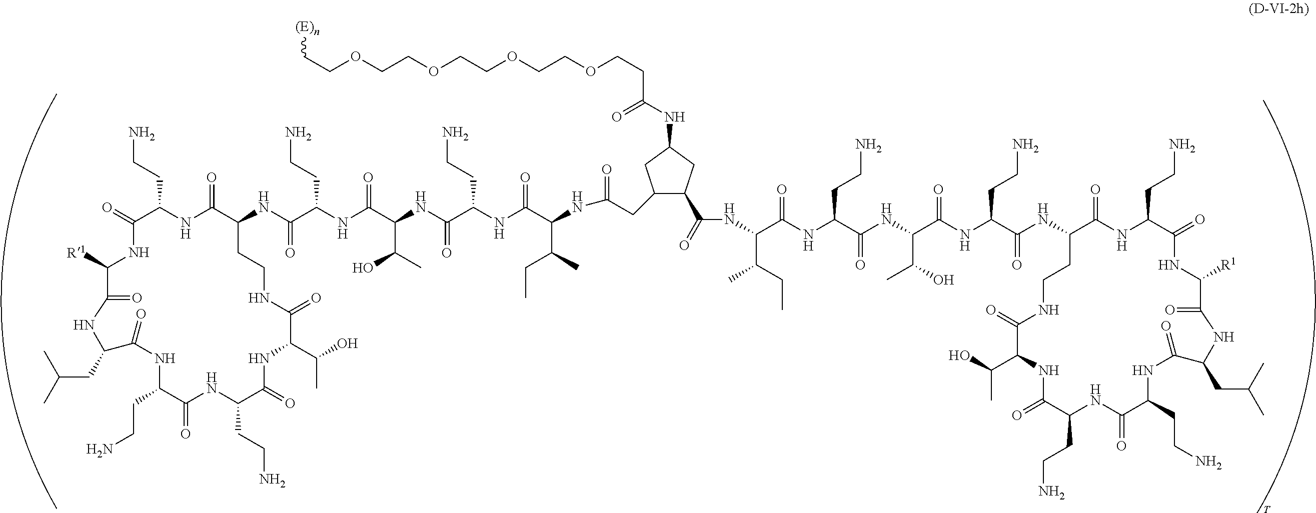

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-2i):

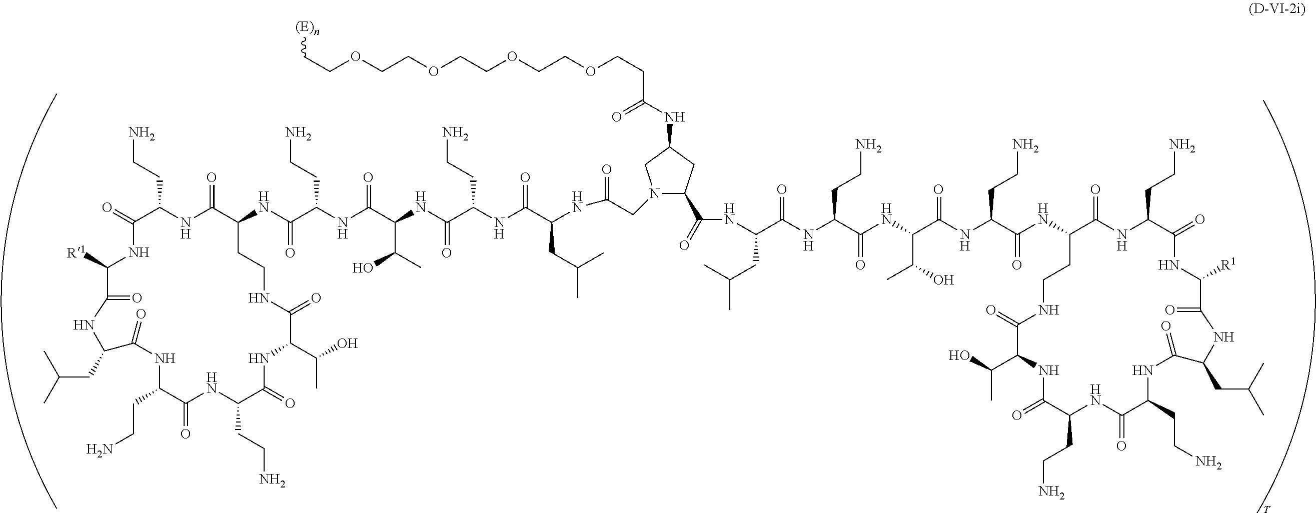

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-2j):

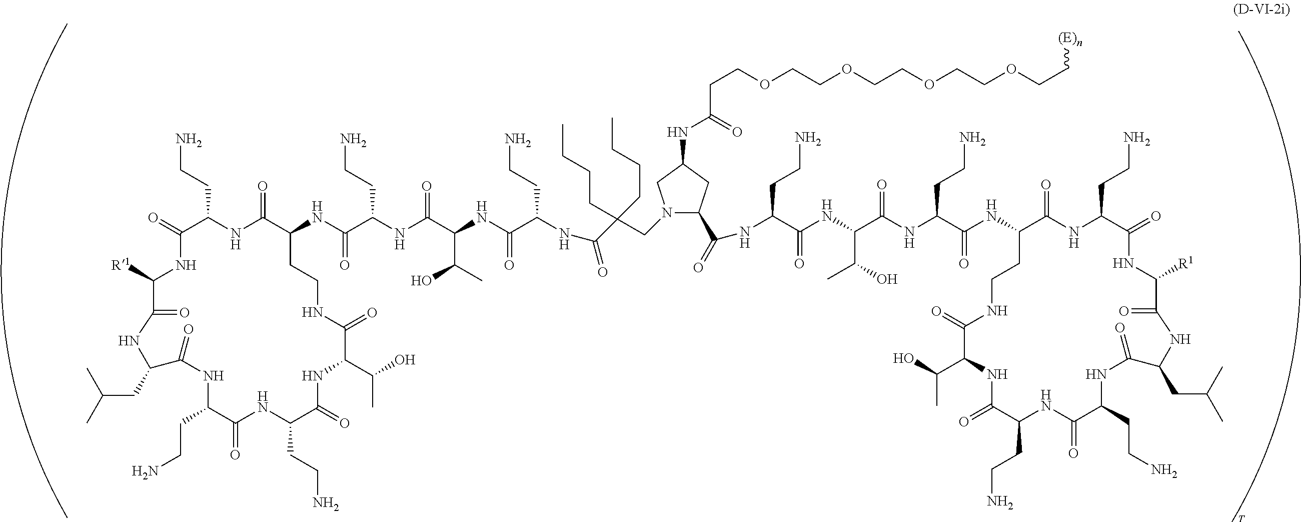

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-2k):

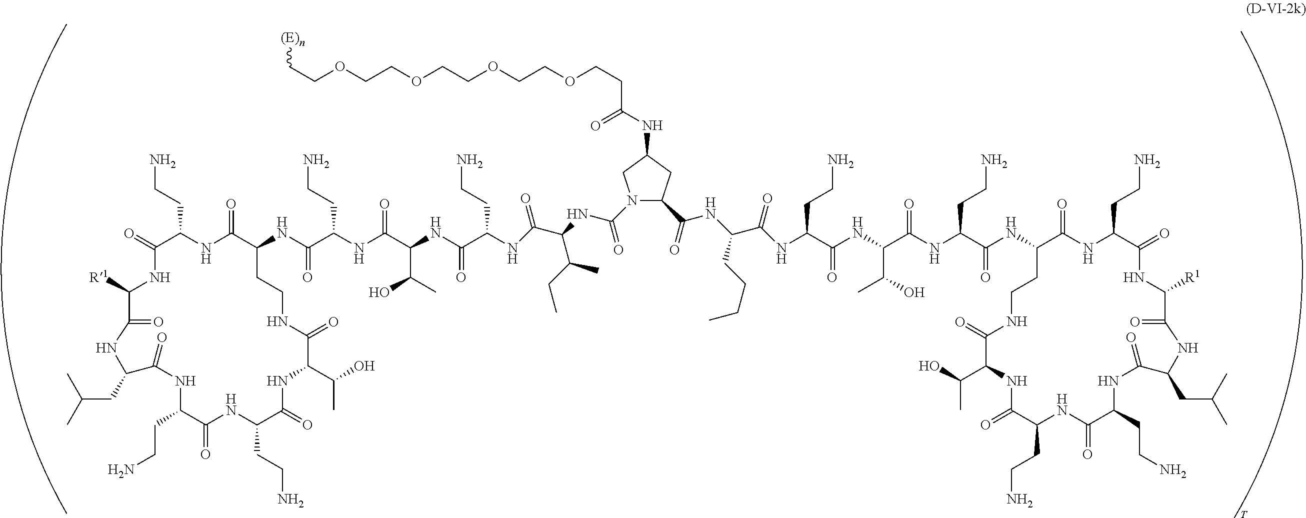

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-2I):

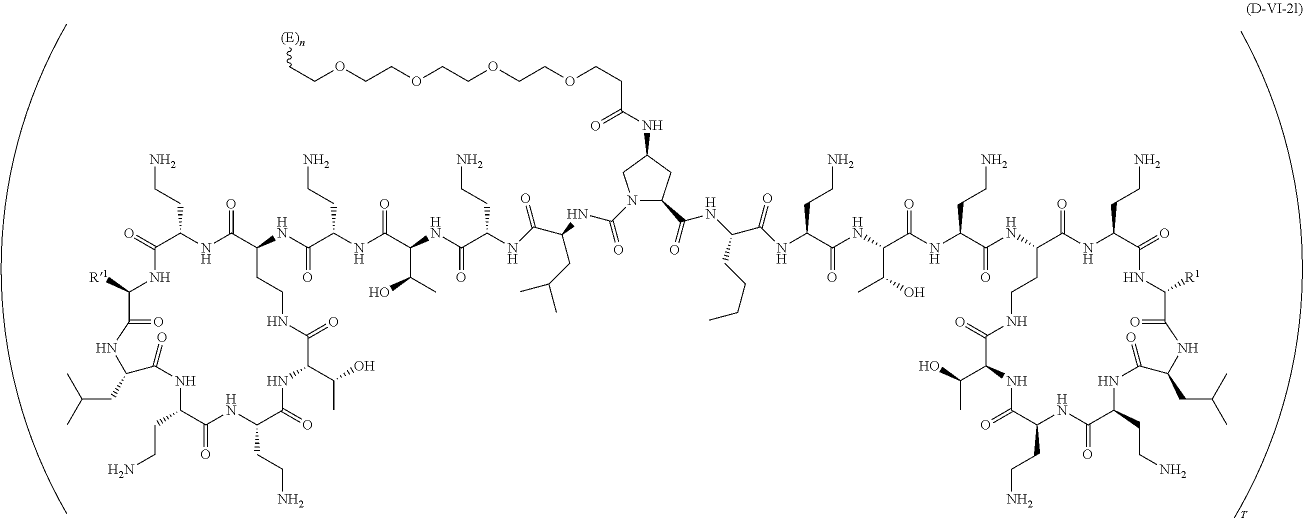

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-2m):

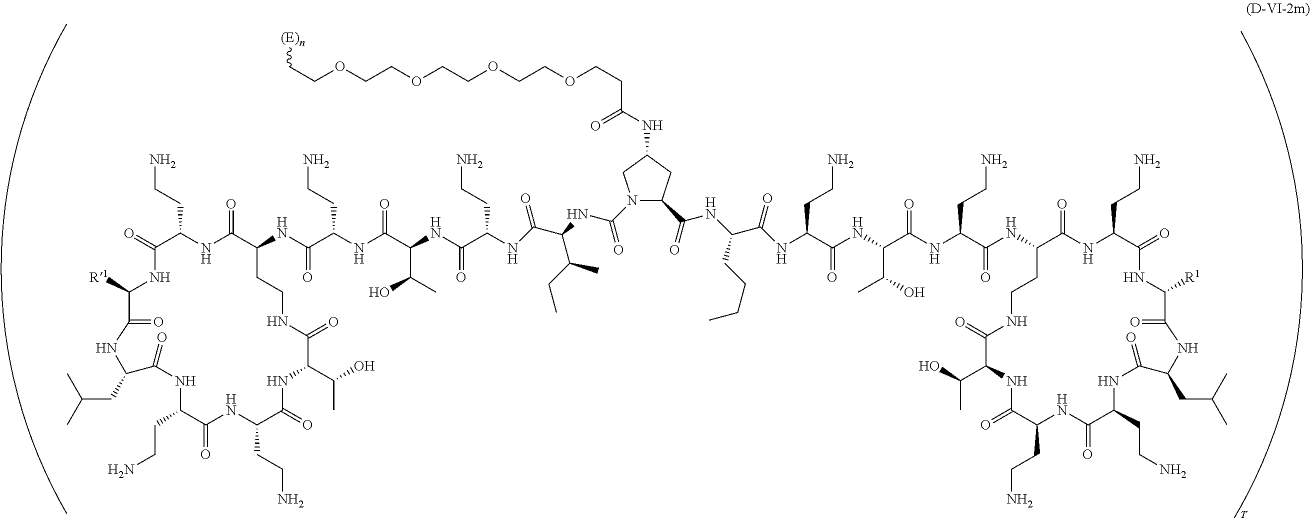

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-2n):

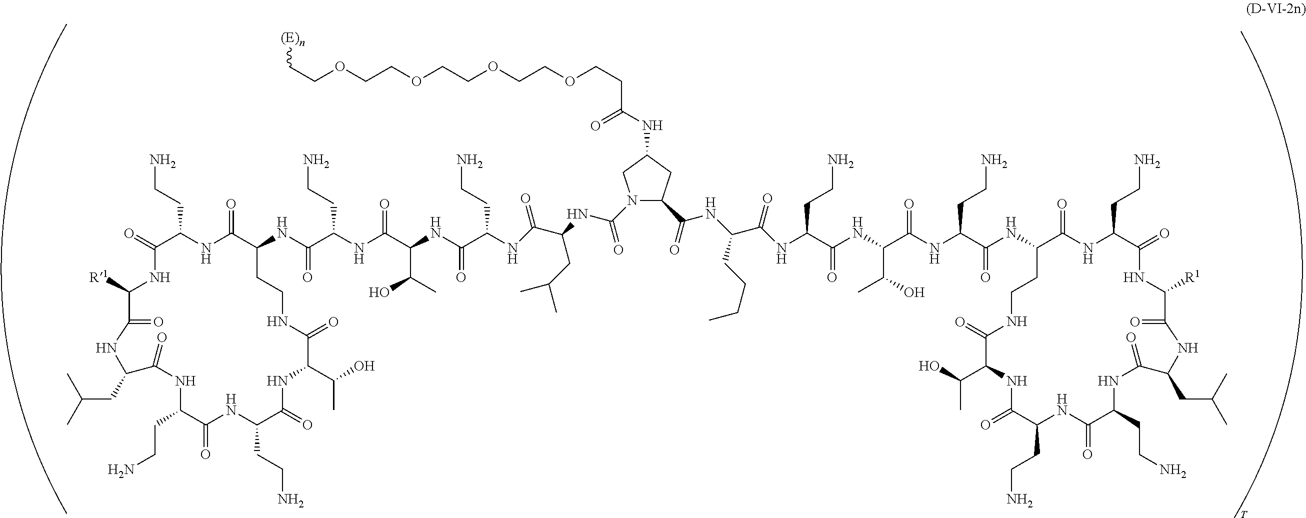

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-2o):

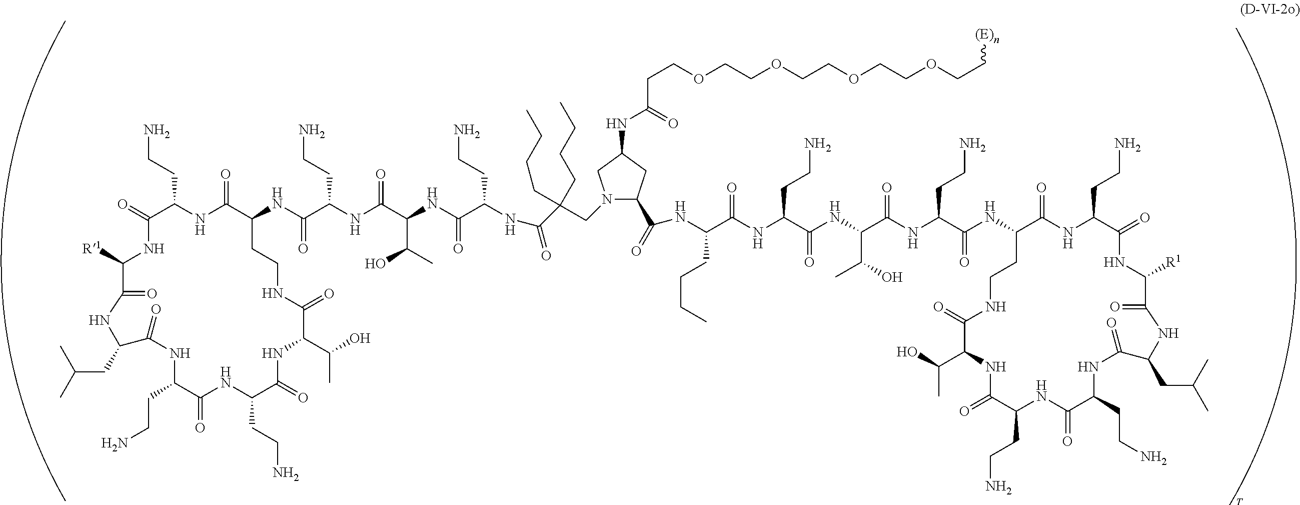

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-2p):

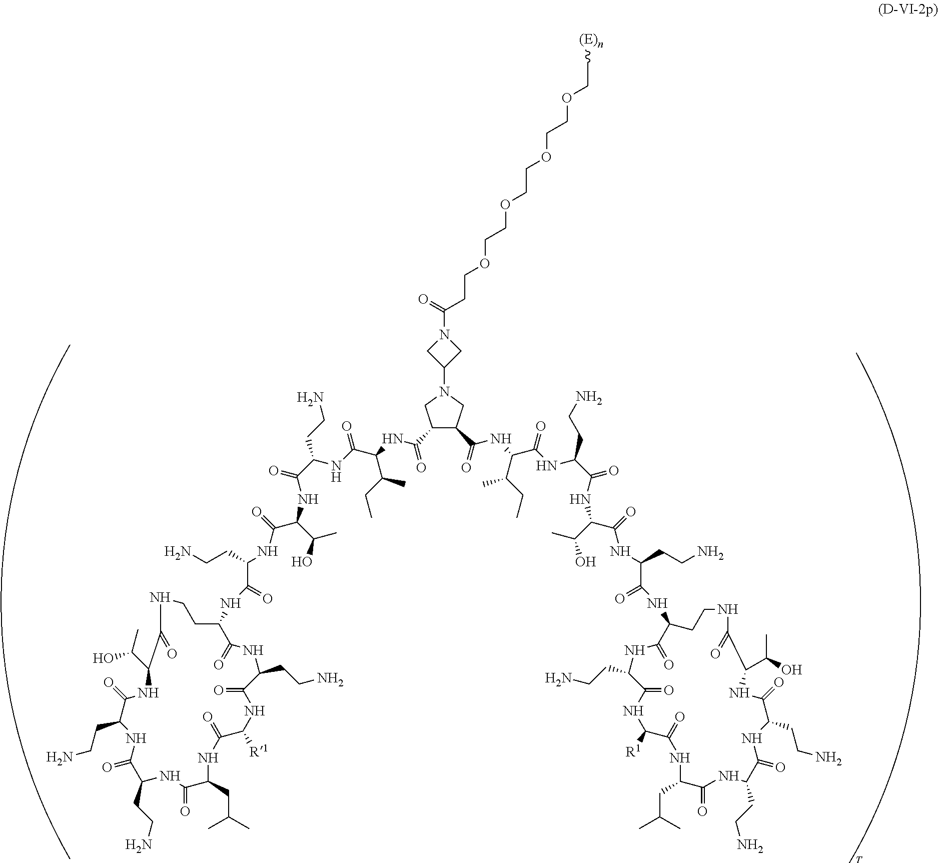

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-2q):

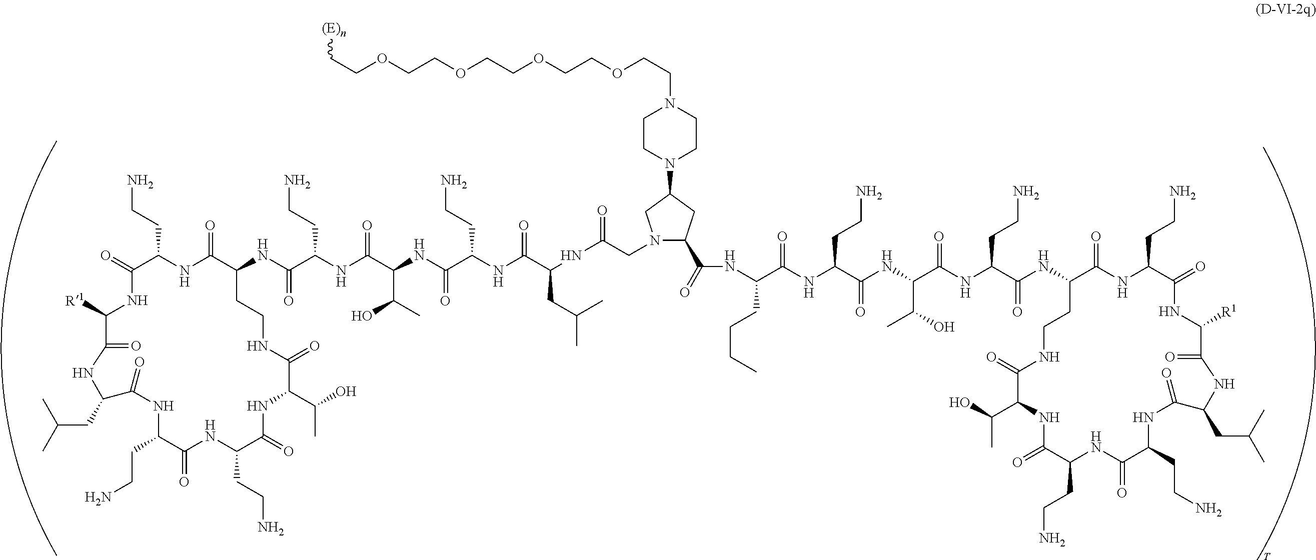

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-2r):

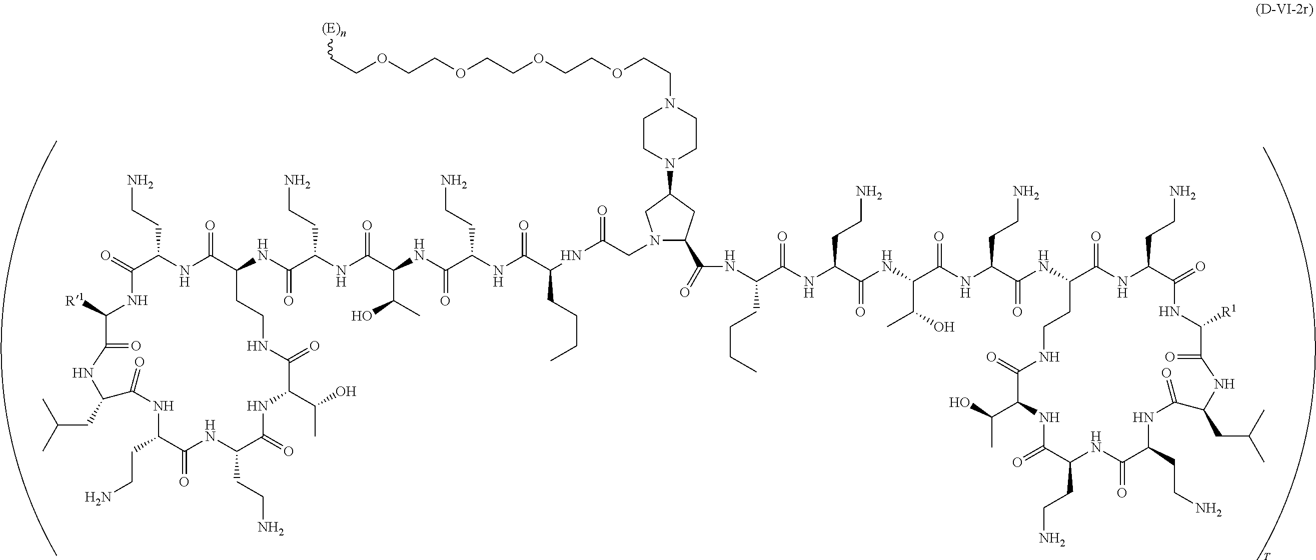

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-2s):

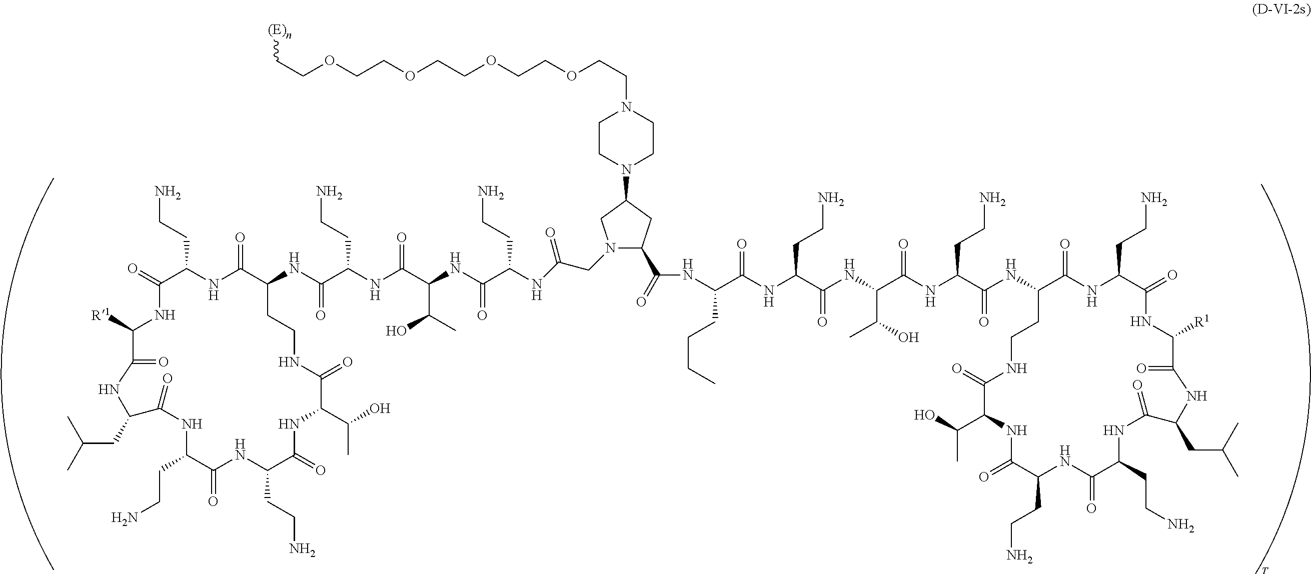

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-2t):

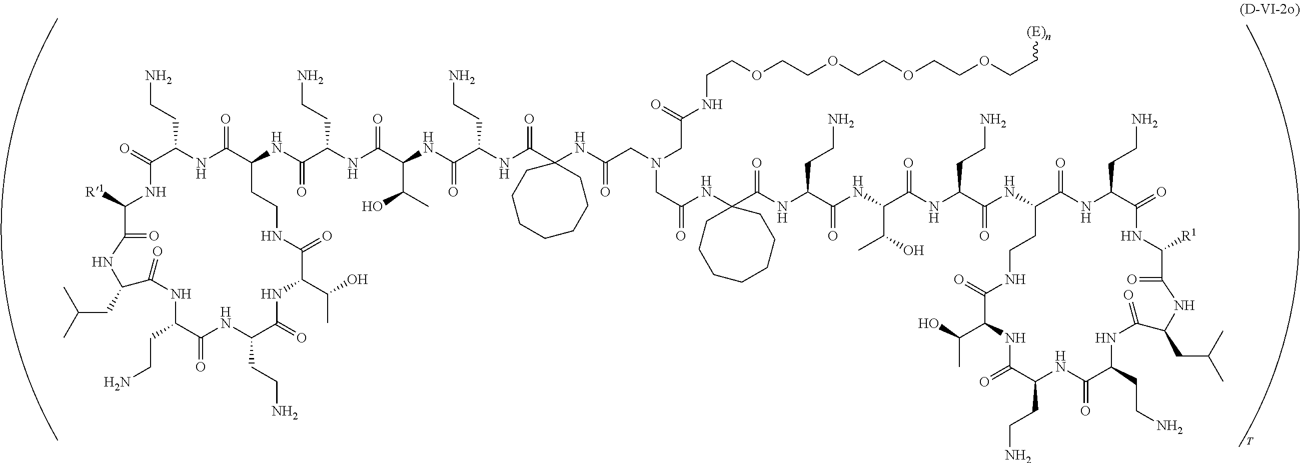

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-2u):

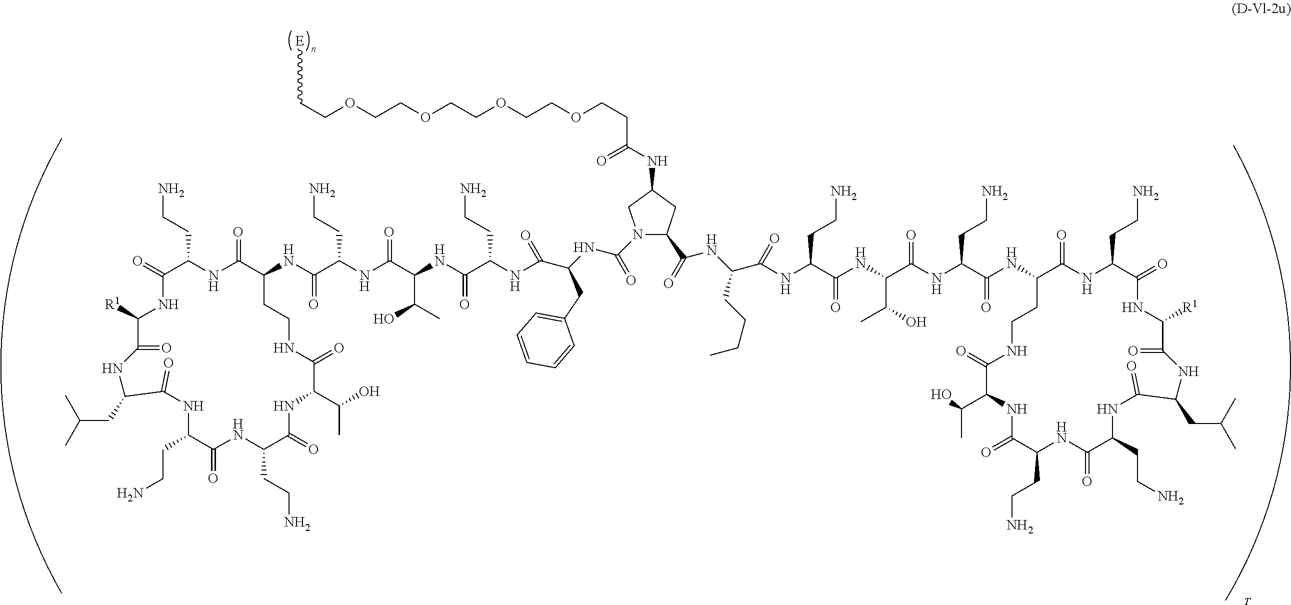

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-2v):

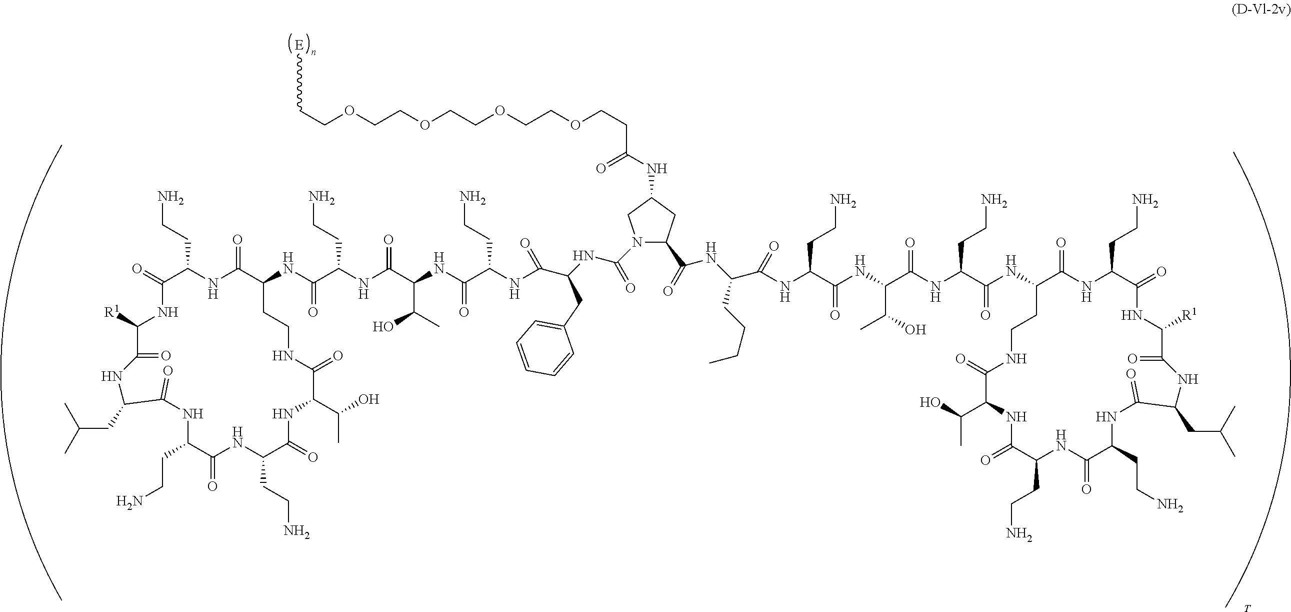

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-2w):

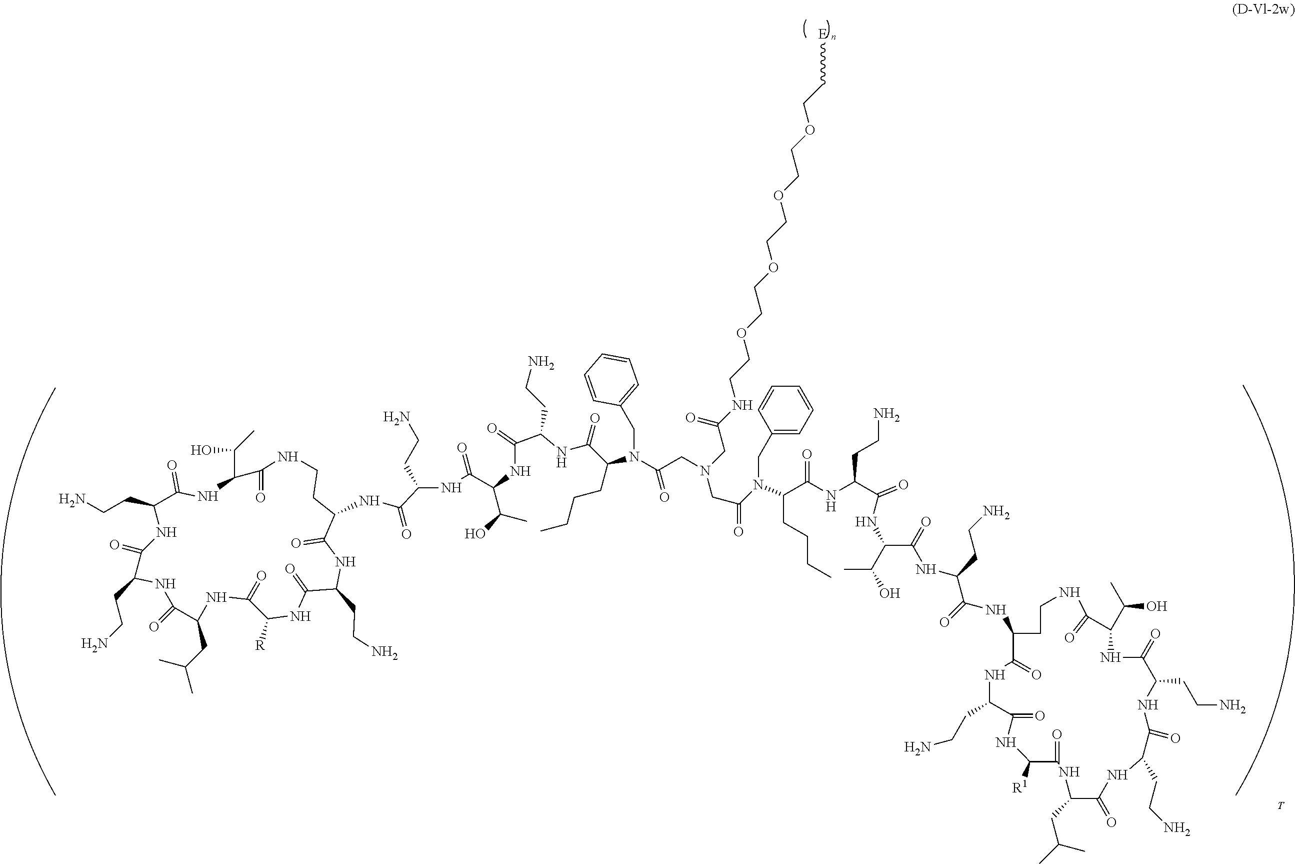

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-2x):

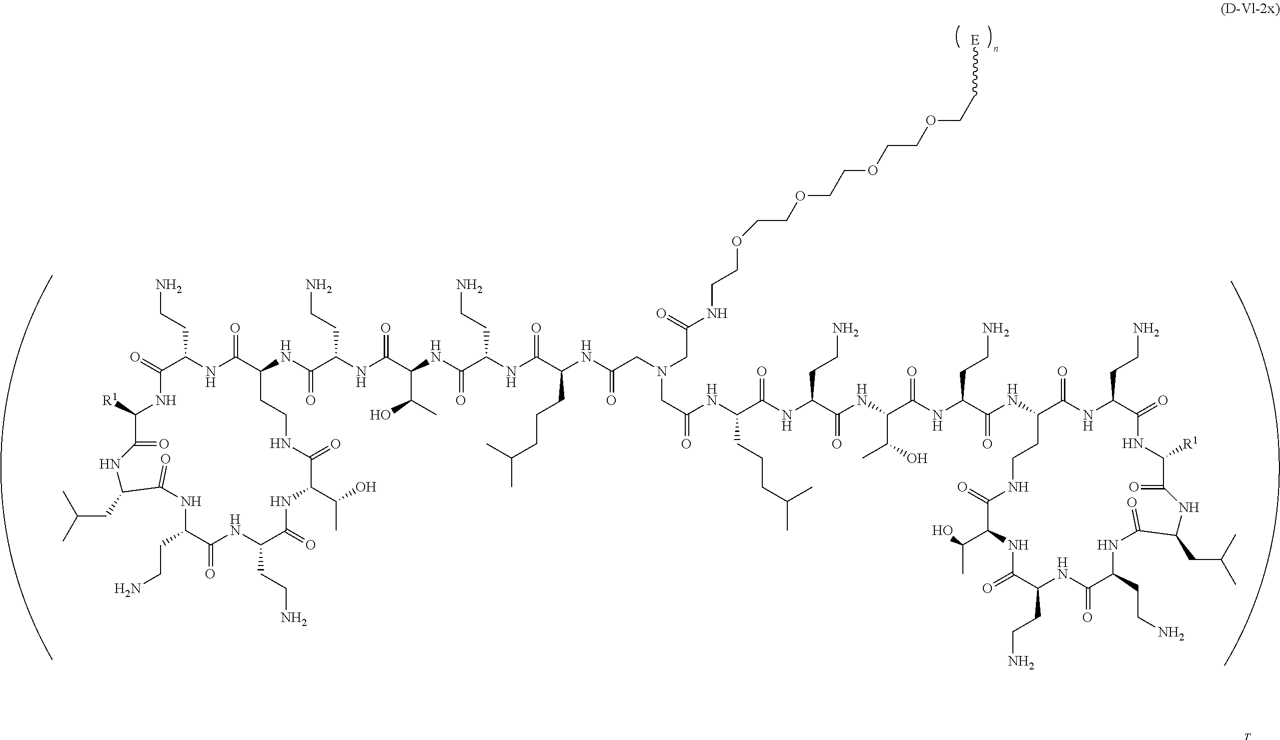

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-2y):

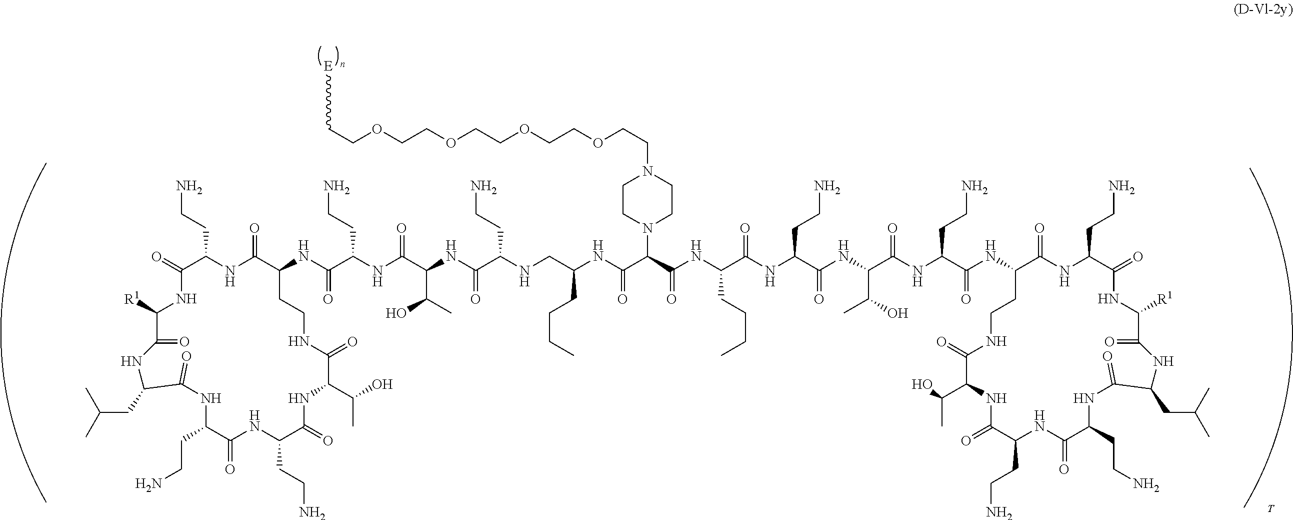

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-2z):

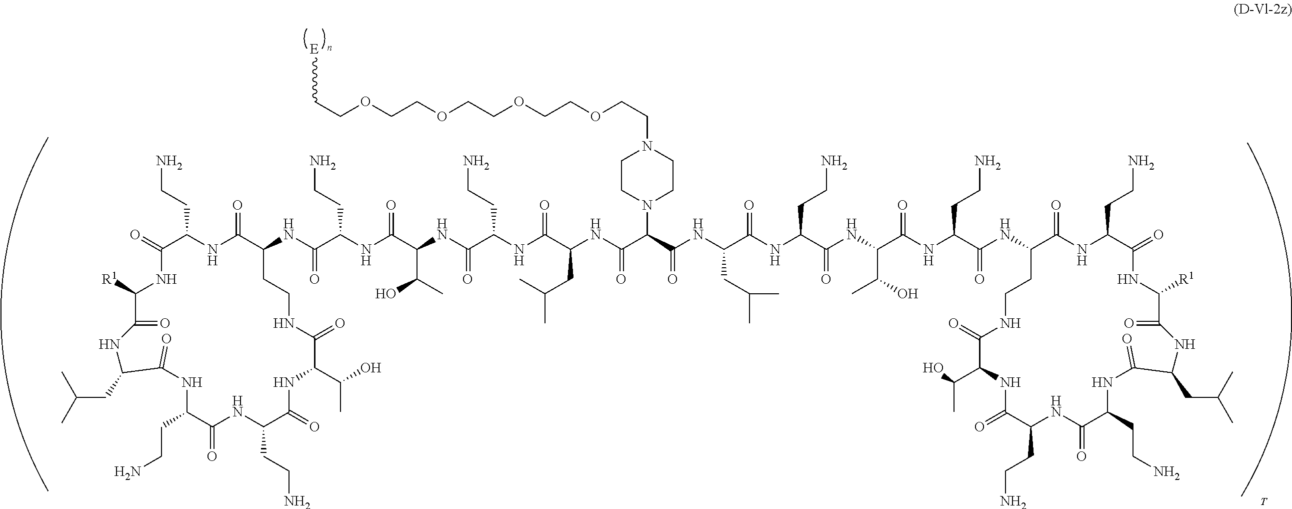

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-2aa):

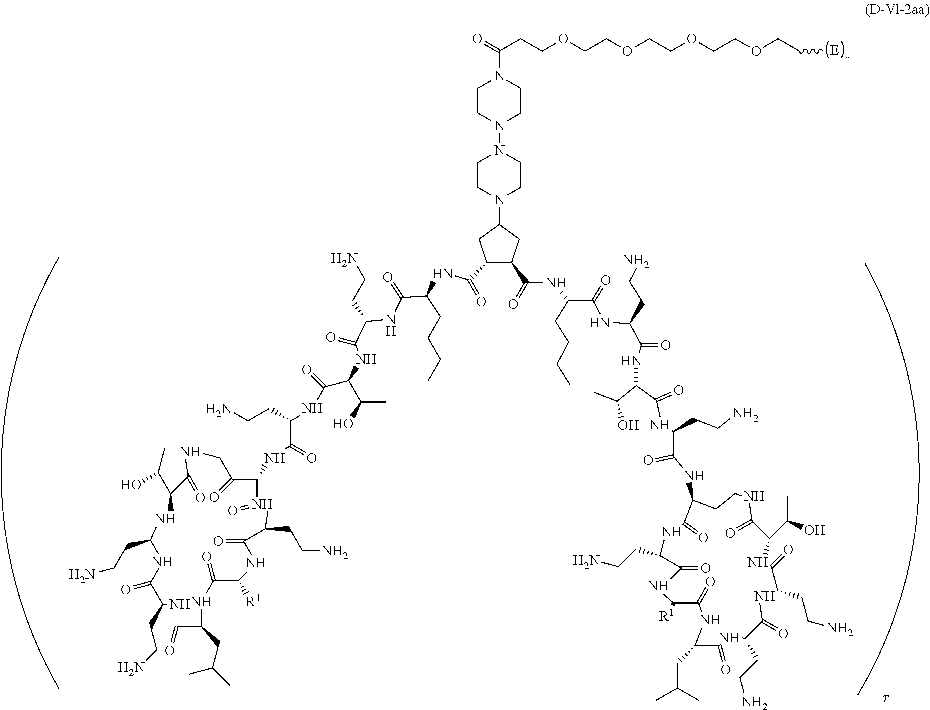

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-2ab):

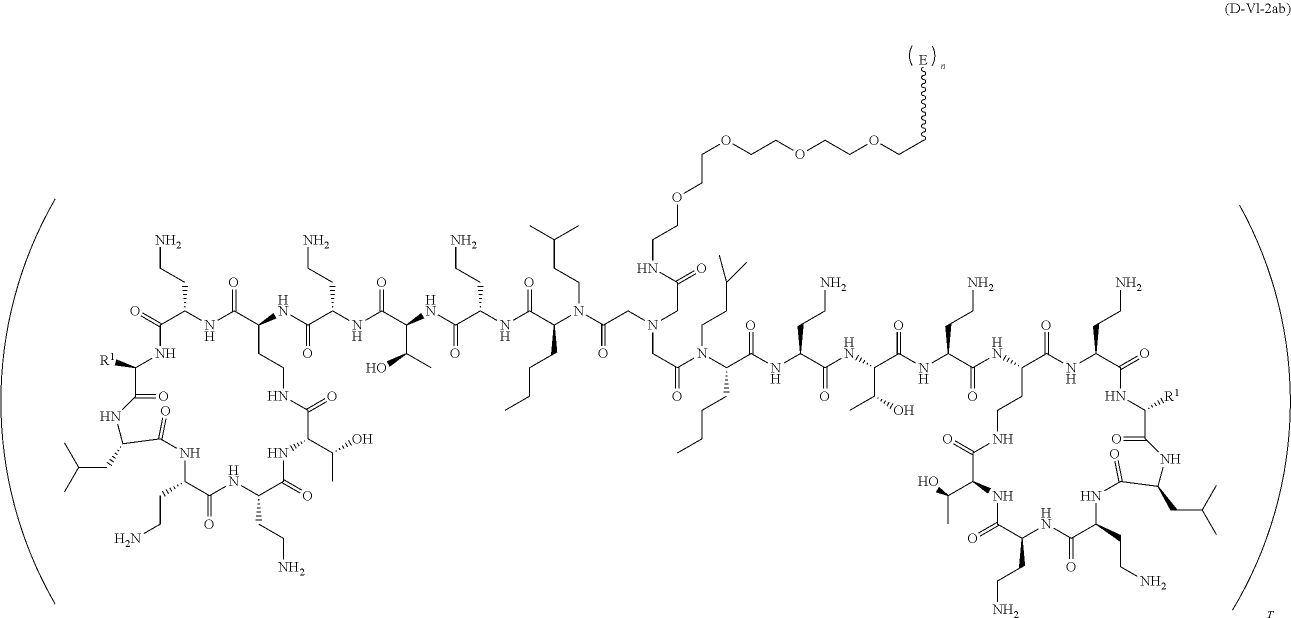

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-2ac):

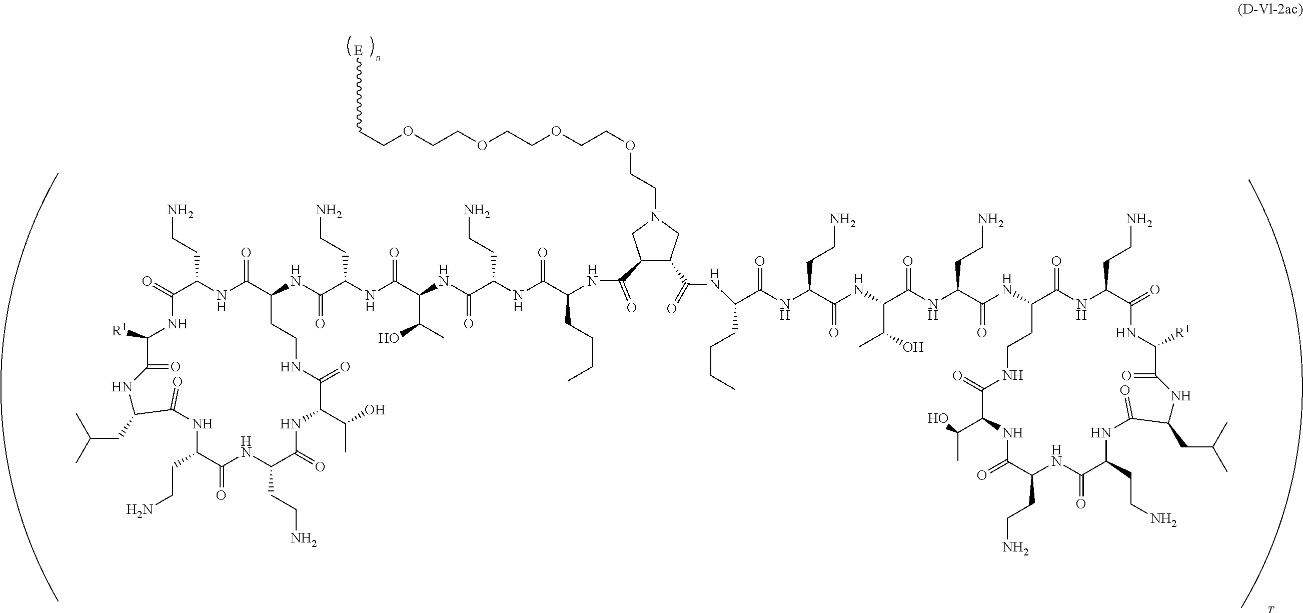

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-2ad):

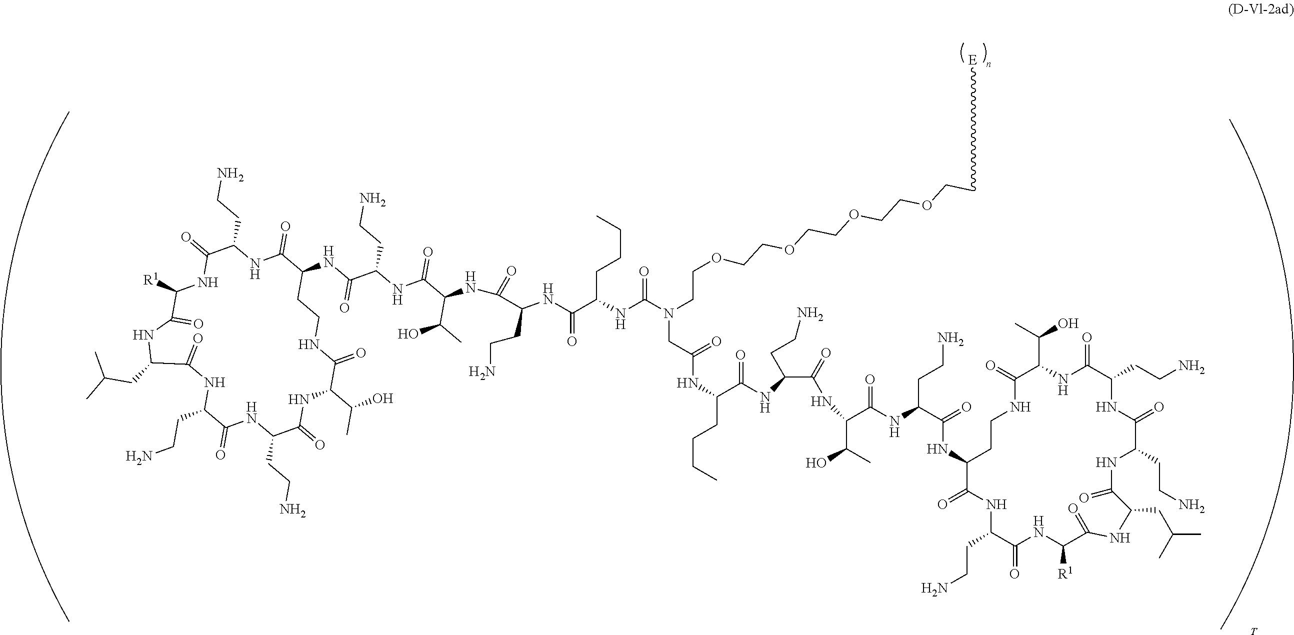

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-V1-2ae):

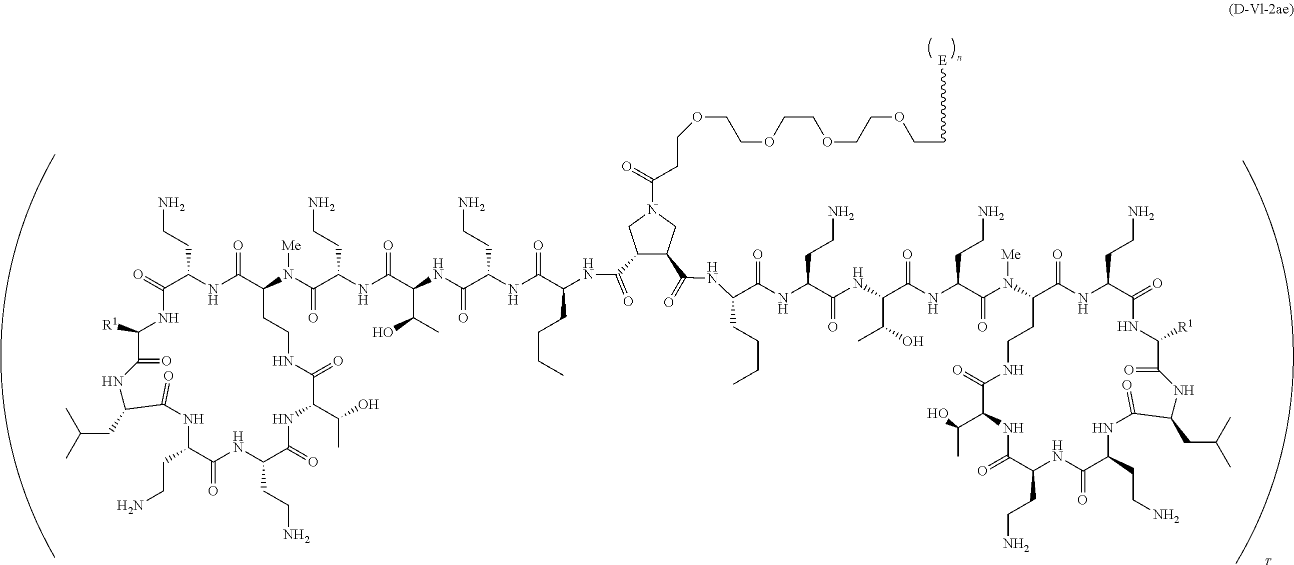

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-2af):

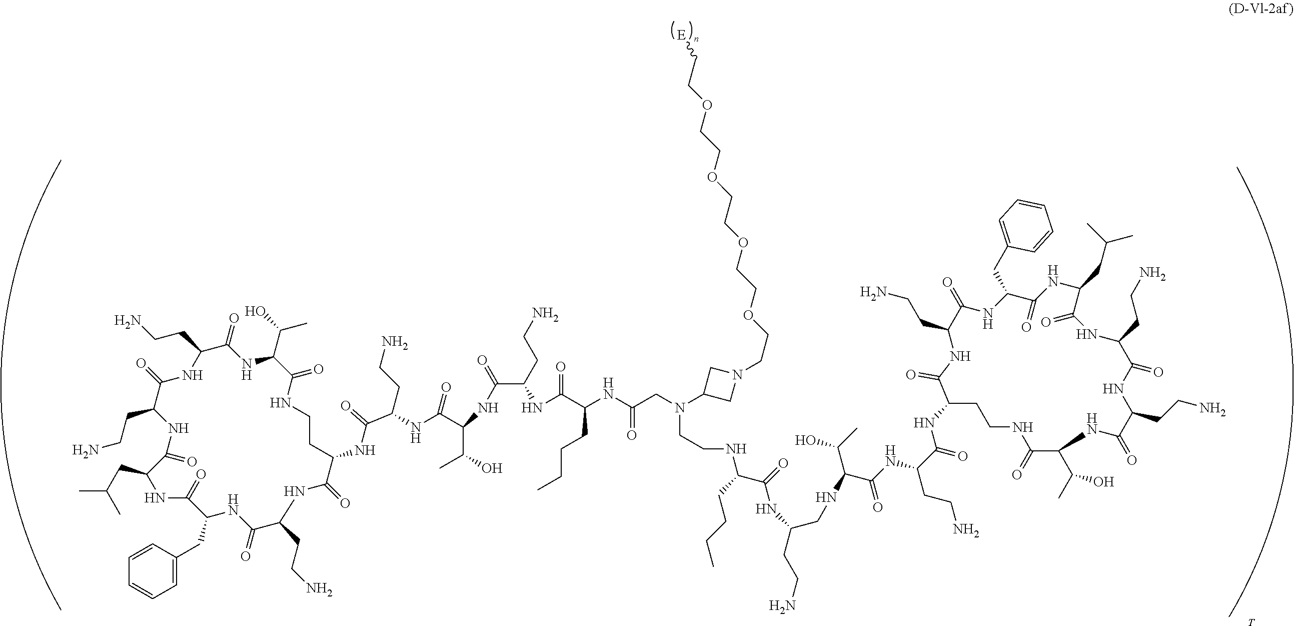

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-2ag):

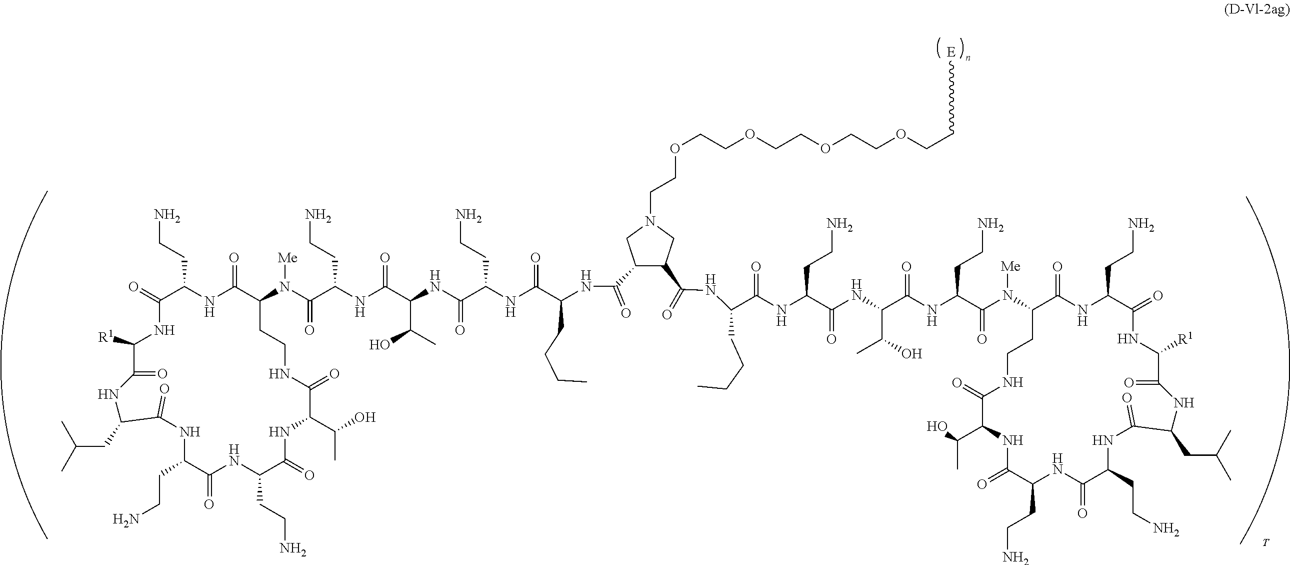

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-2ah):

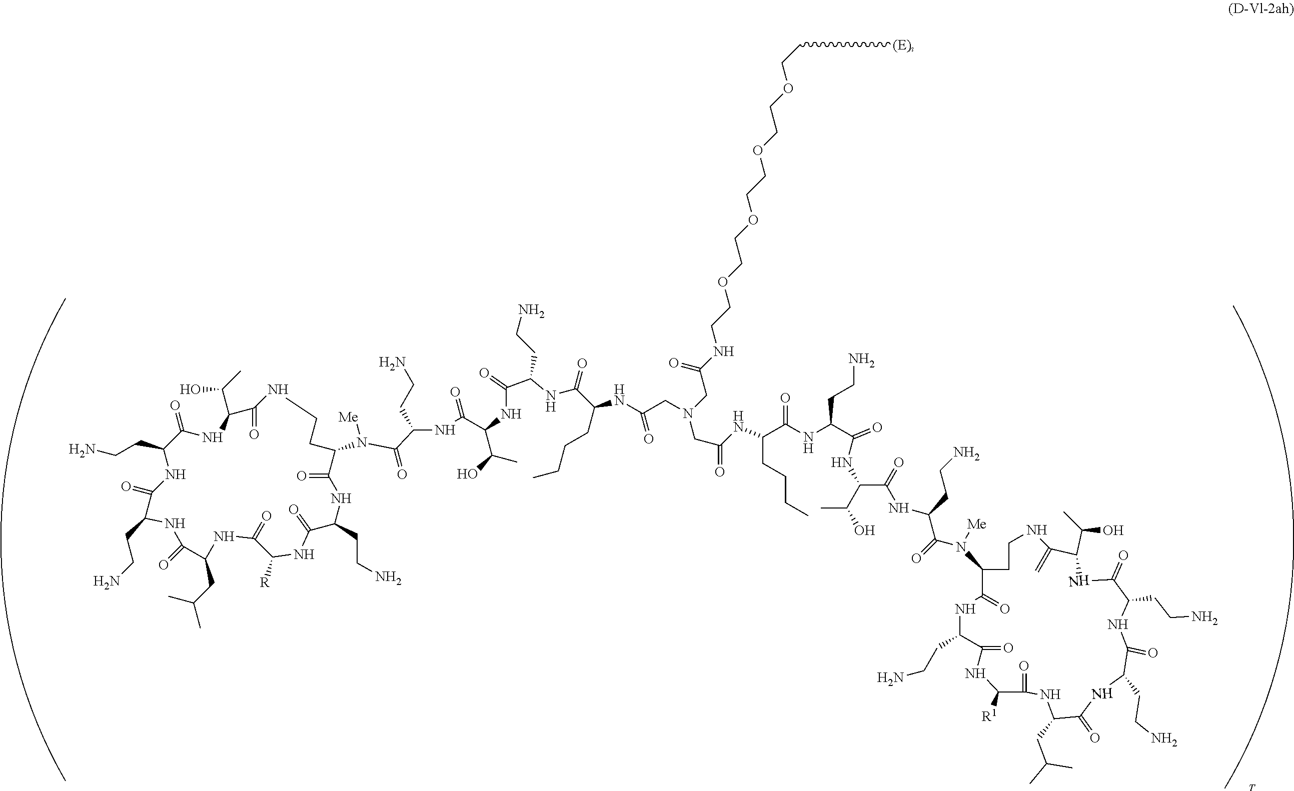

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-A-2ai):

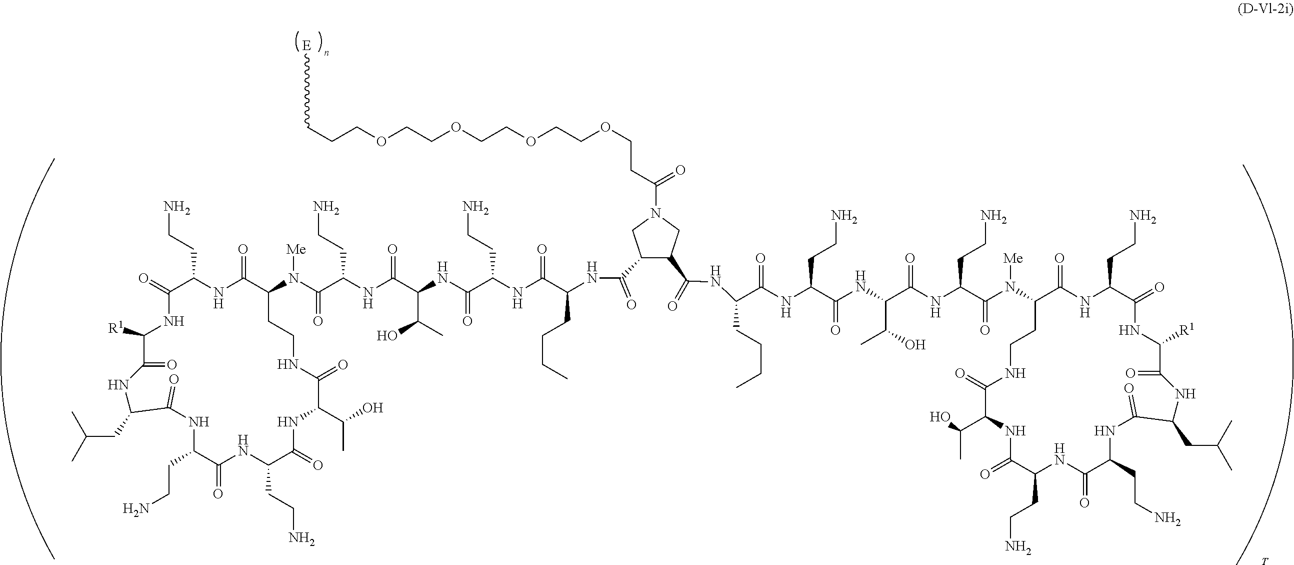

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-3):

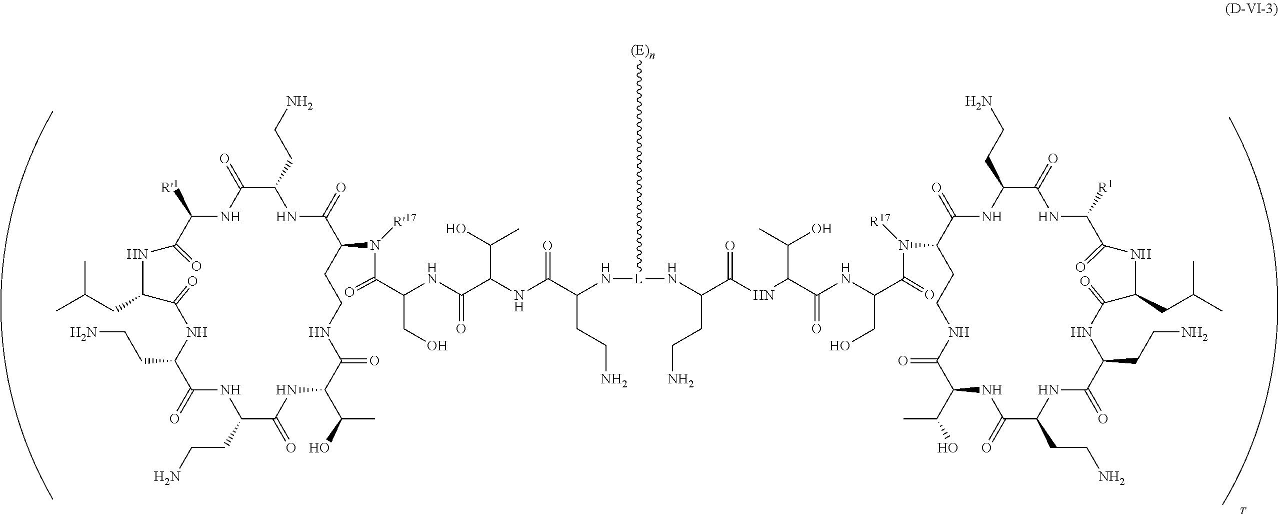

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-4):

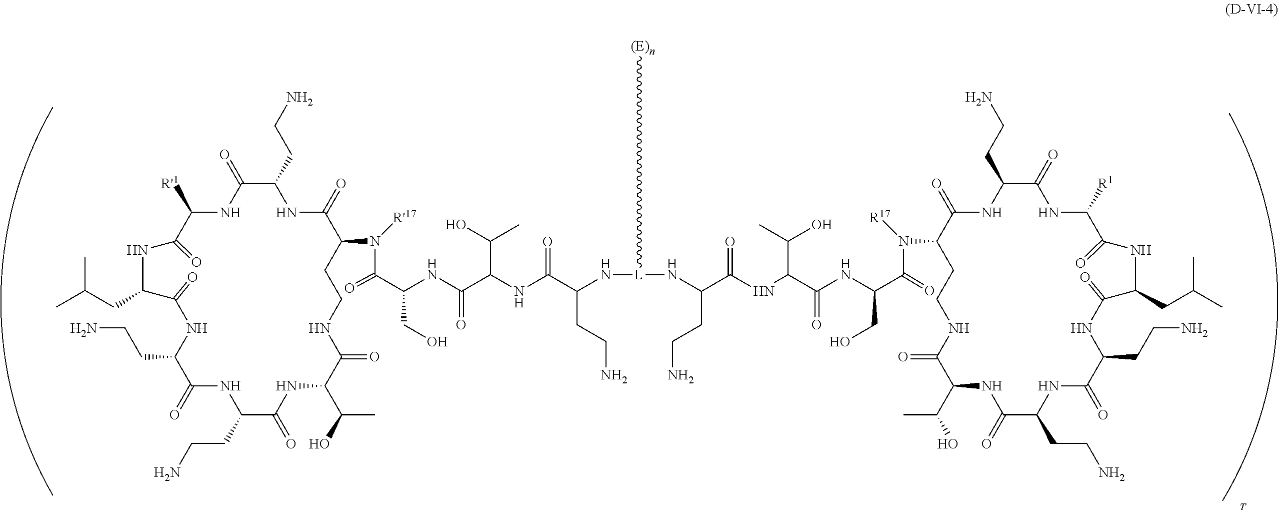

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-5):

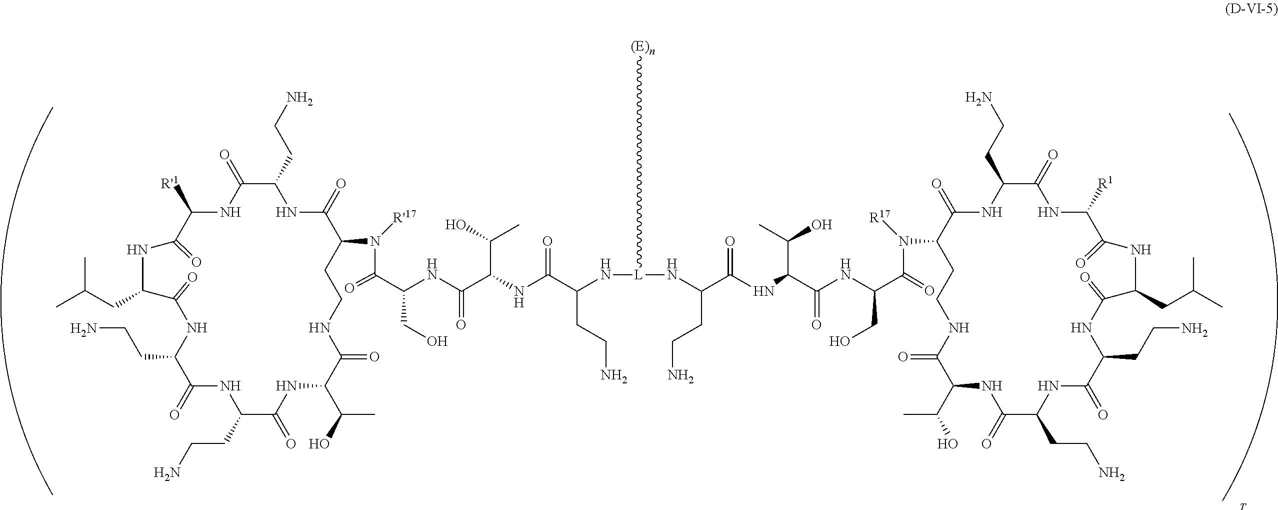

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-6):

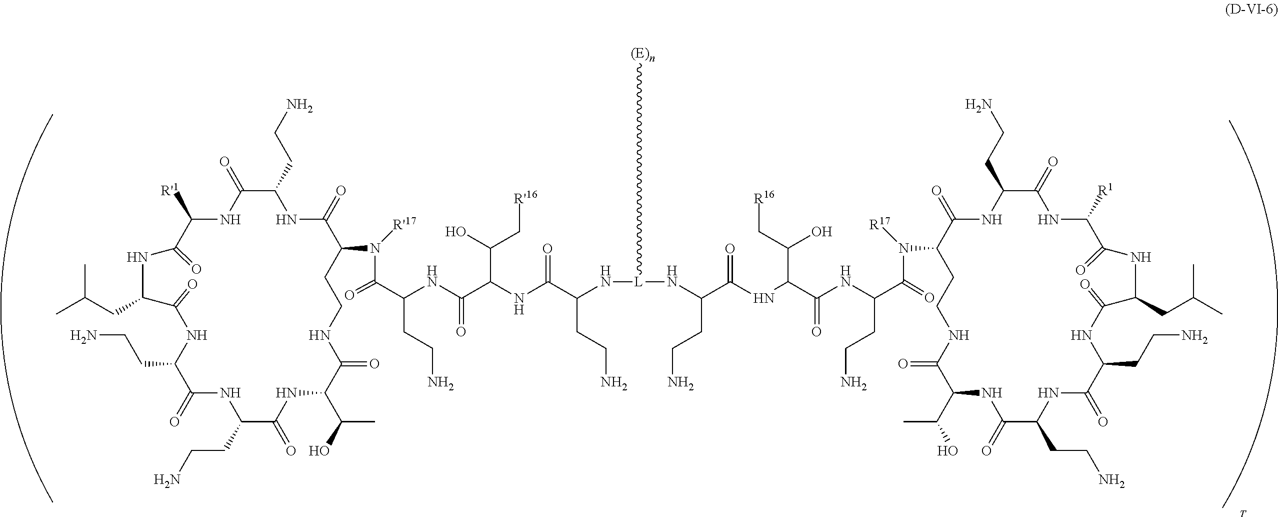

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R′¹⁶ and R¹⁶ is, independently, a lipophilic moiety, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments, the conjugate is described by formula (D-VI-6a):

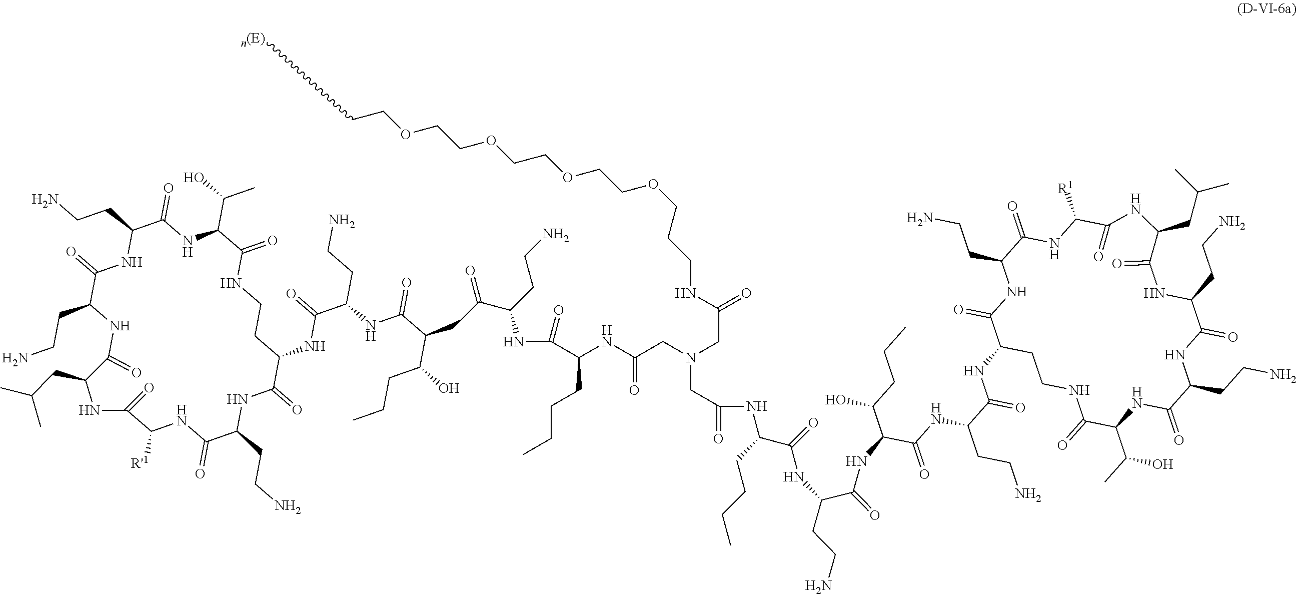

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-6b):

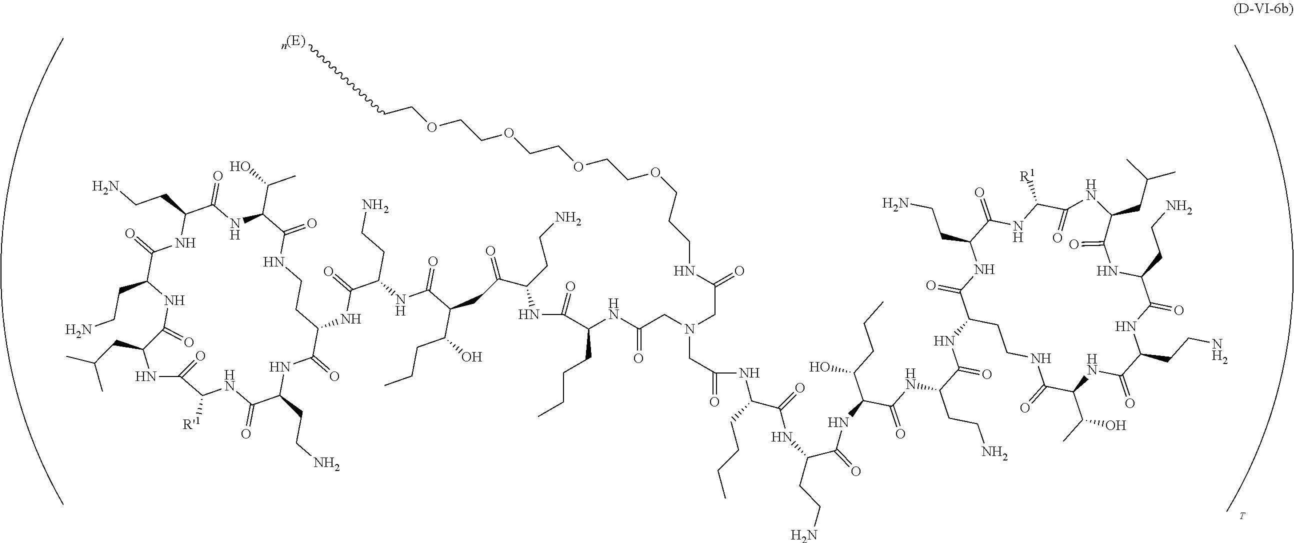

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-6c):

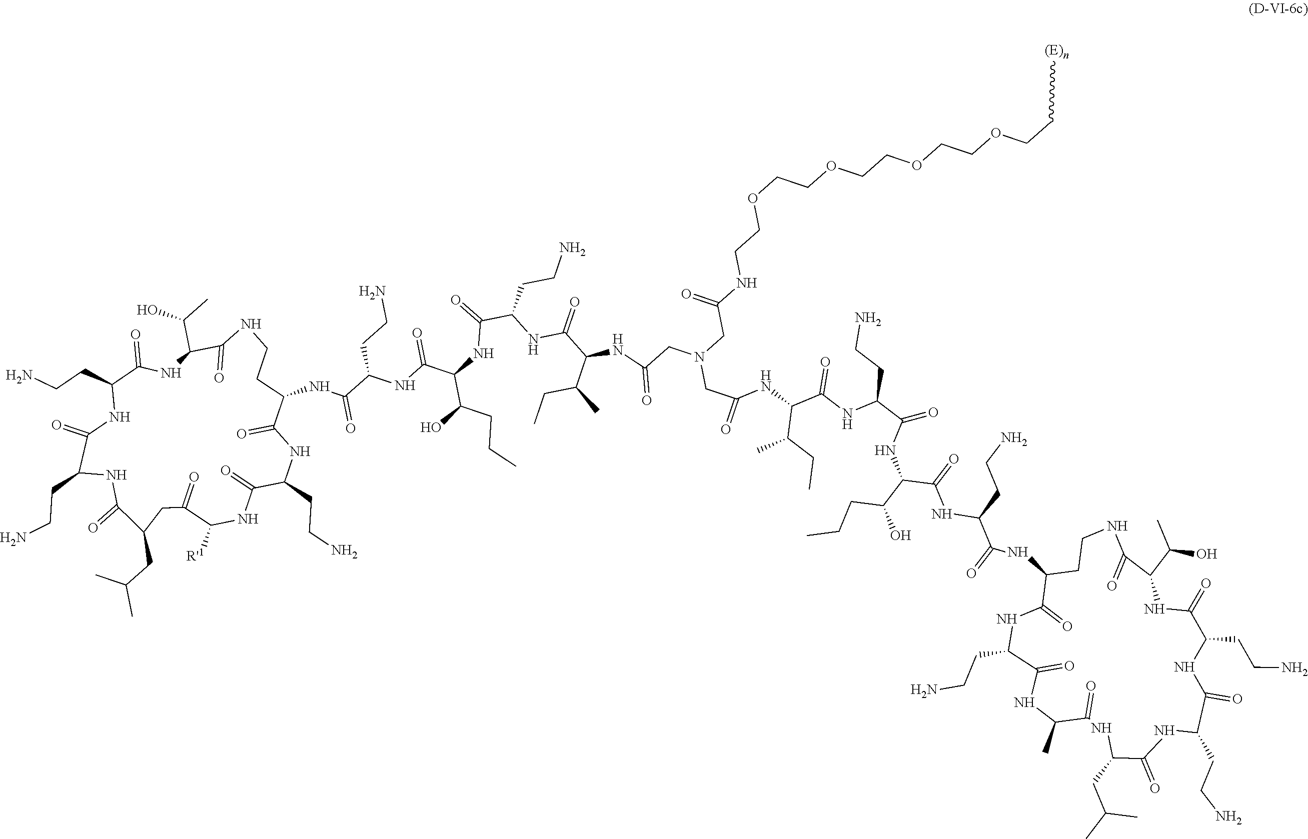

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In some embodiments, the conjugate is described by formula (D-VI-6d):

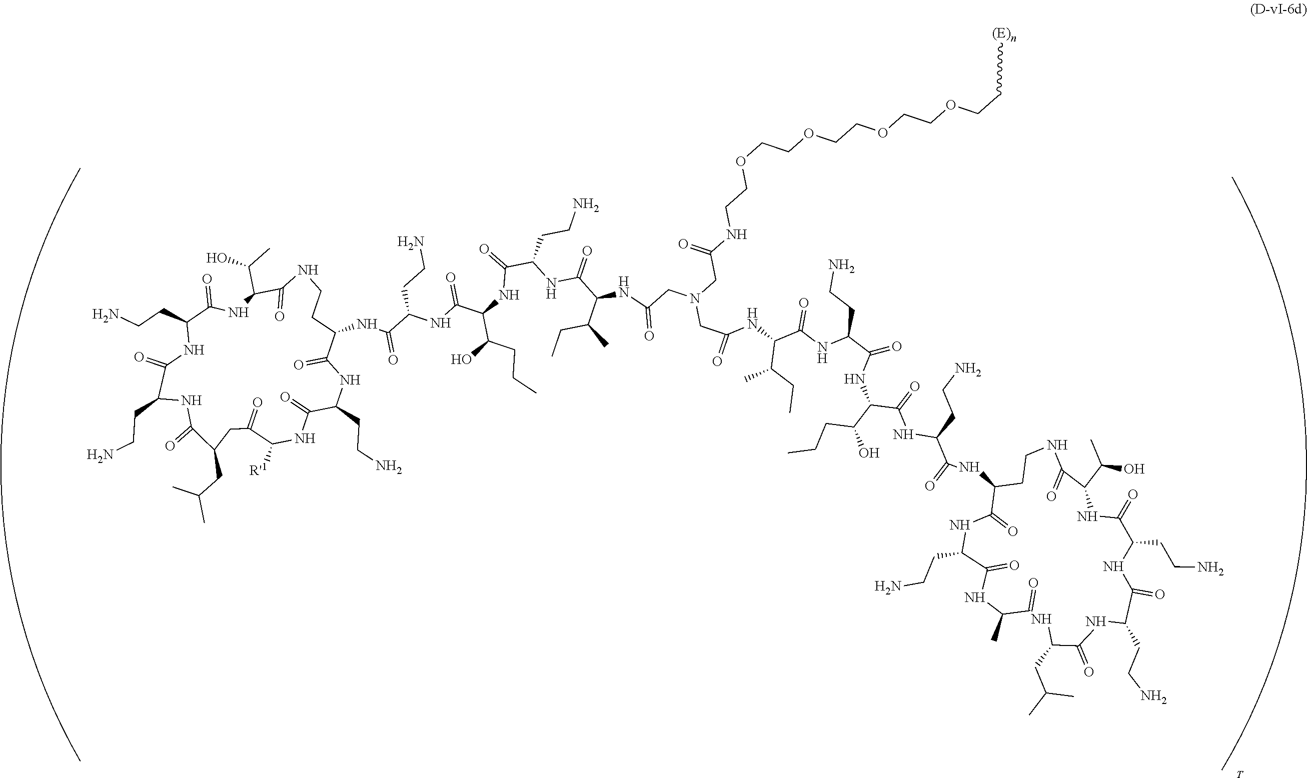

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-6e):

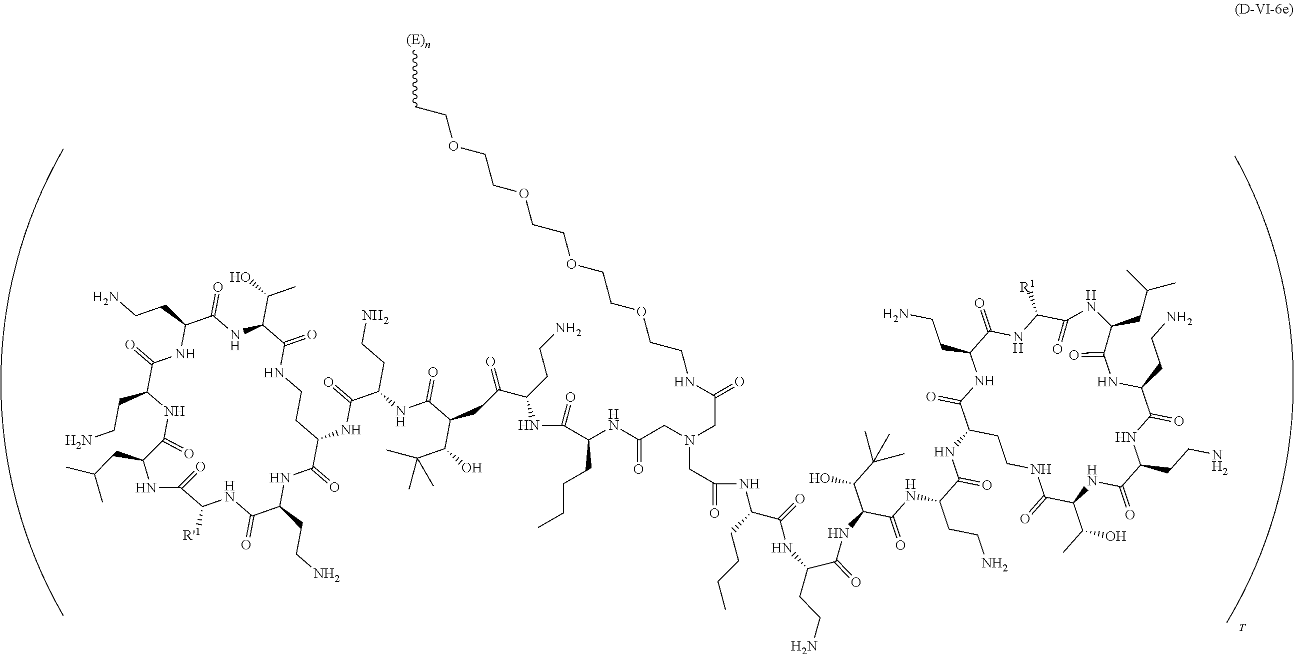

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-6f):

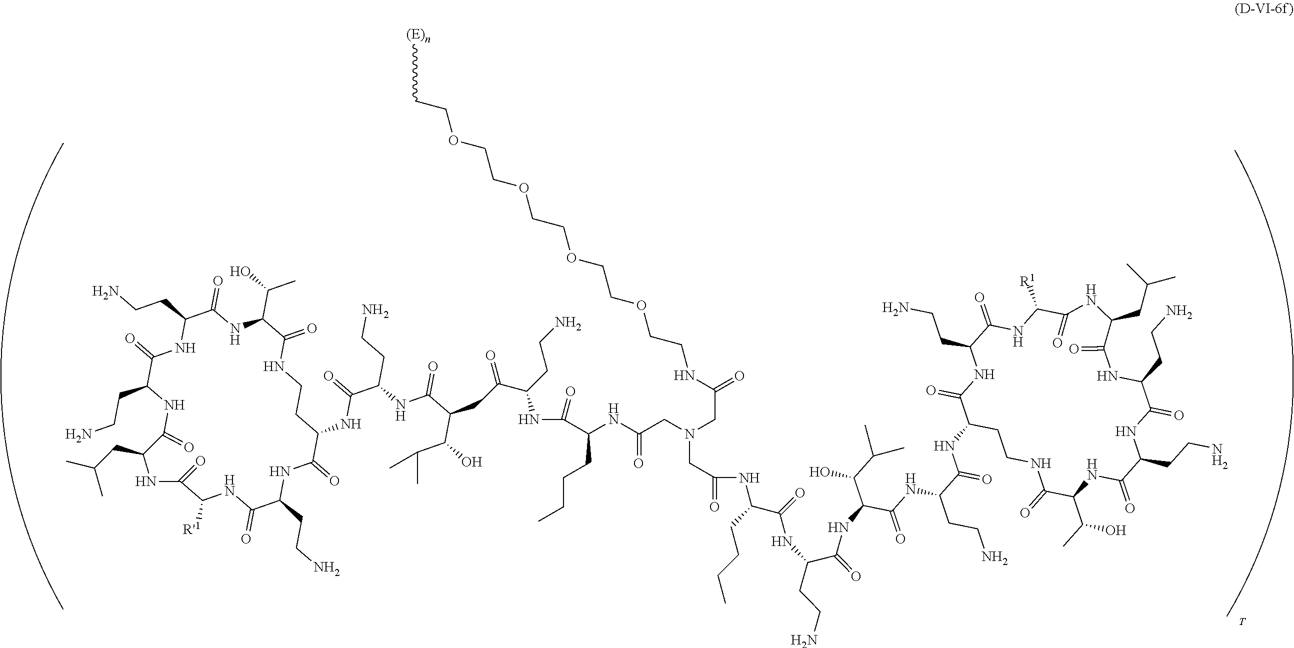

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In some embodiments, the conjugate is described by formula (D-VI-6g):

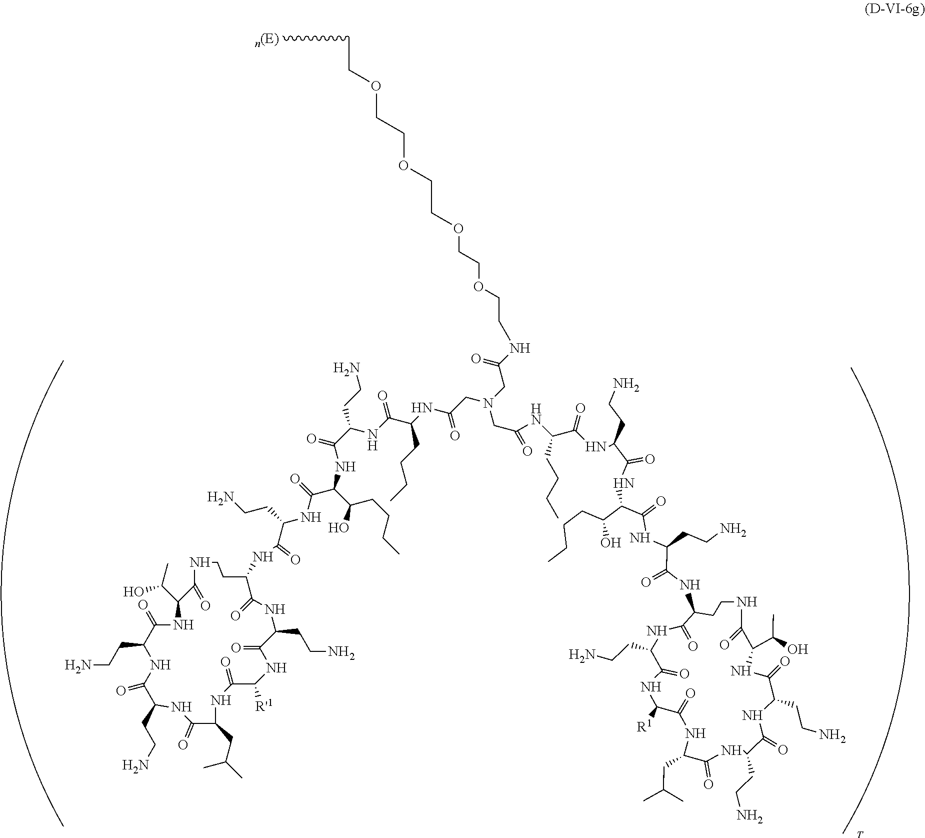

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In some embodiments, the conjugate is described by formula (D-VI-6h):

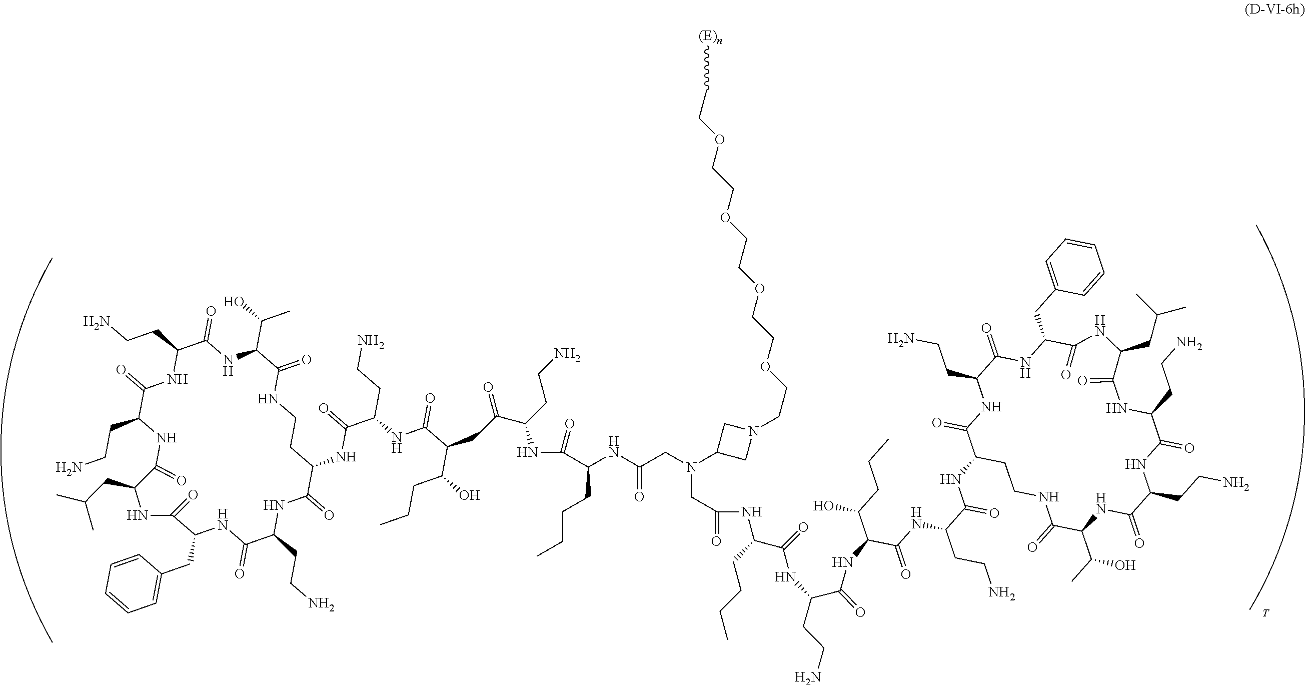

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-V-6i):

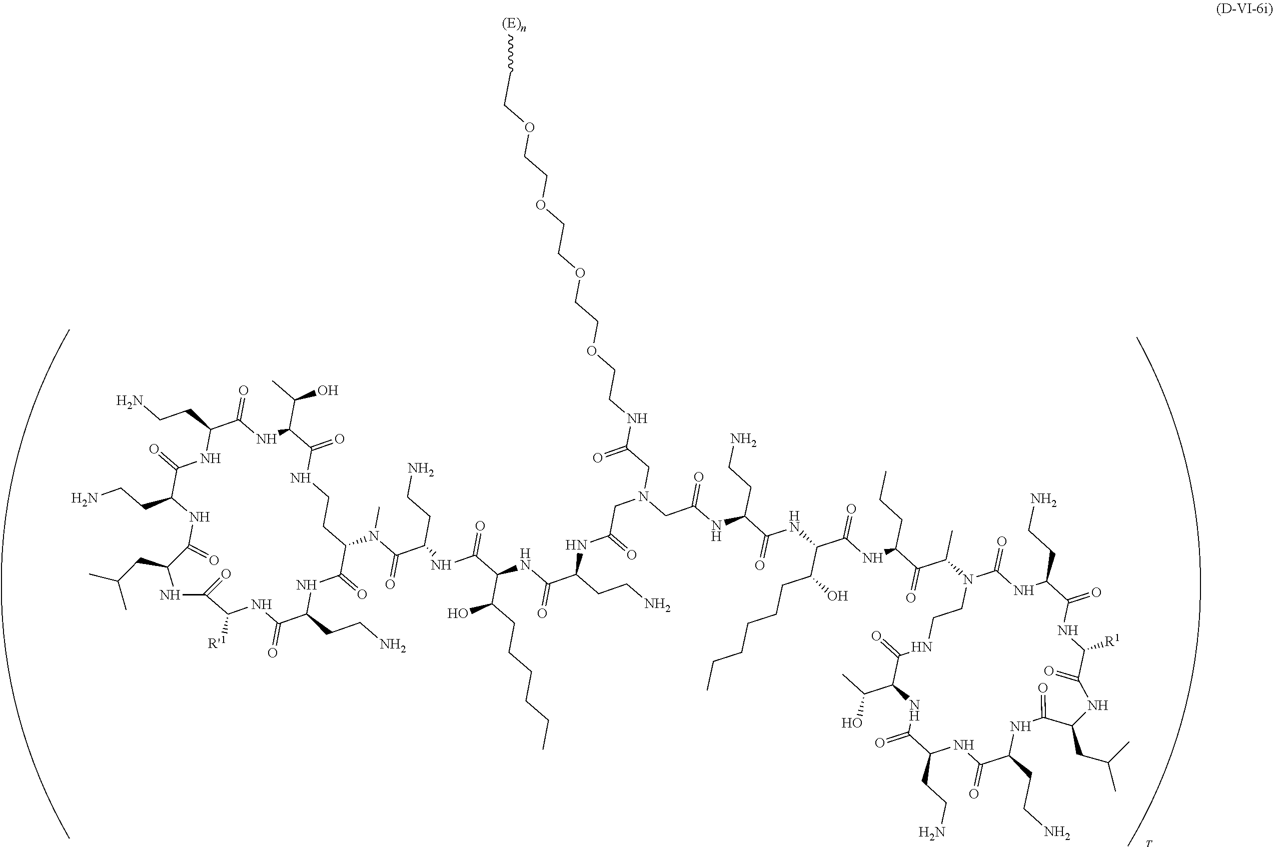

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-6j):

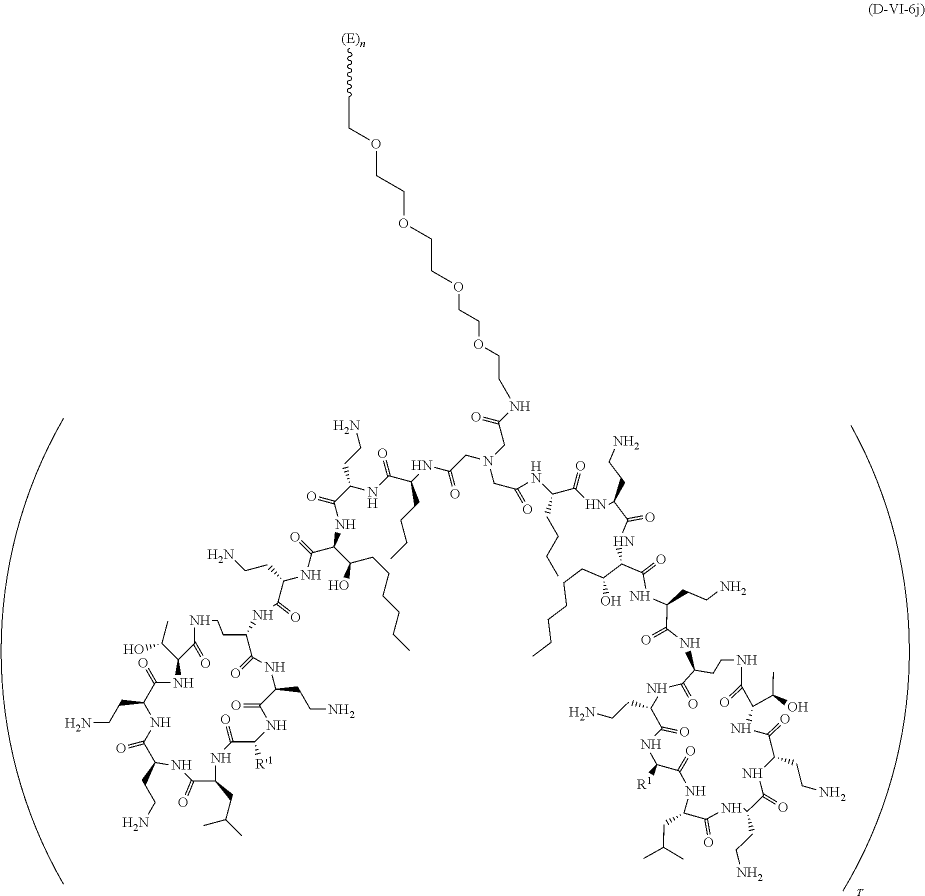

wherein each of R¹ and R′¹ is, independently, optionally substituted benzyl. CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-8k):

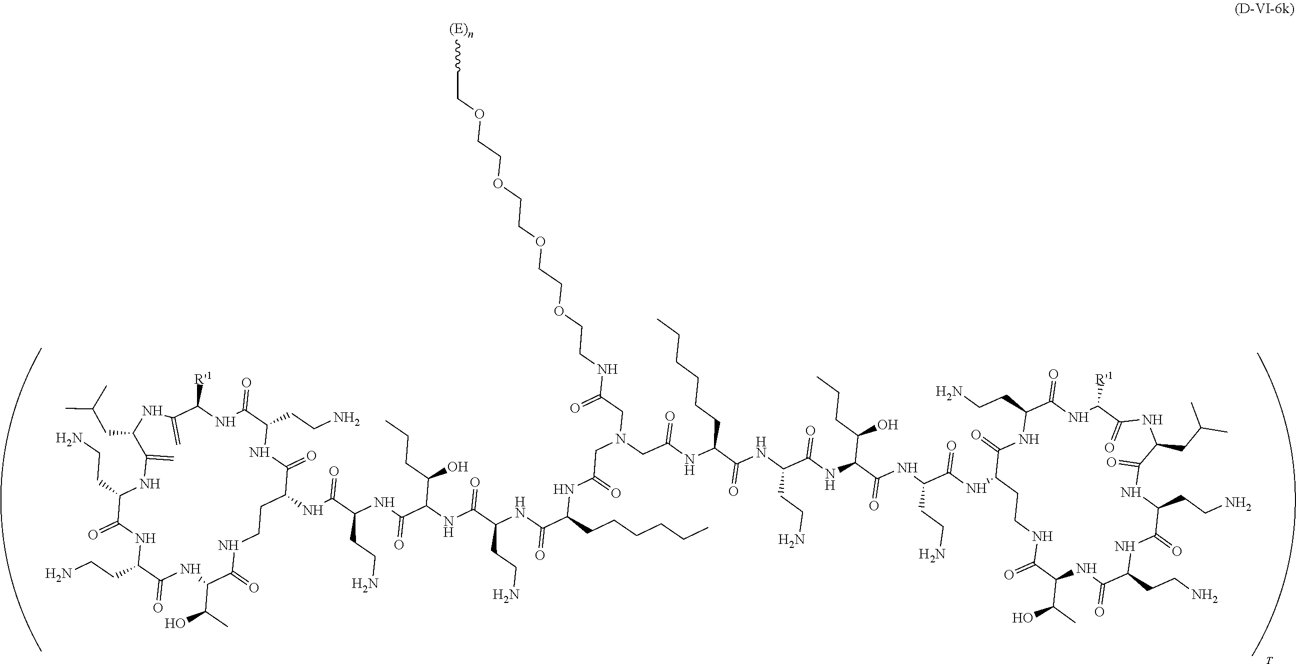

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-6l):

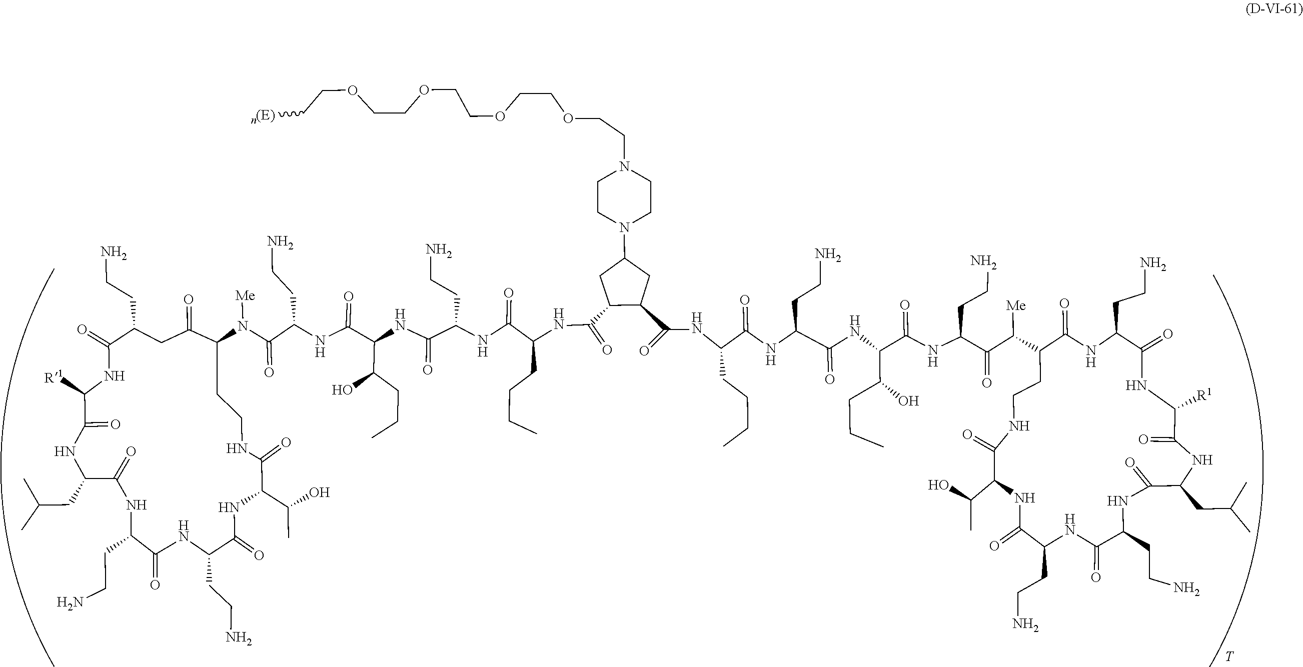

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-6m):

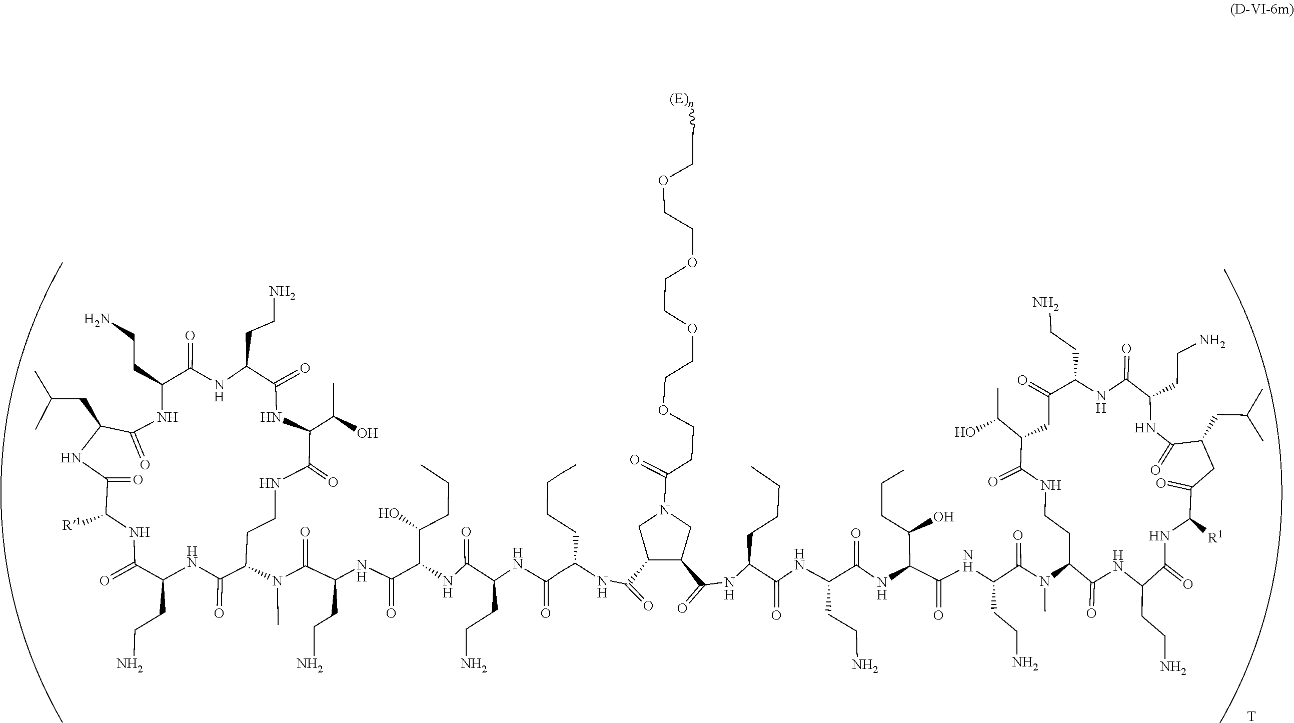

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-6n):

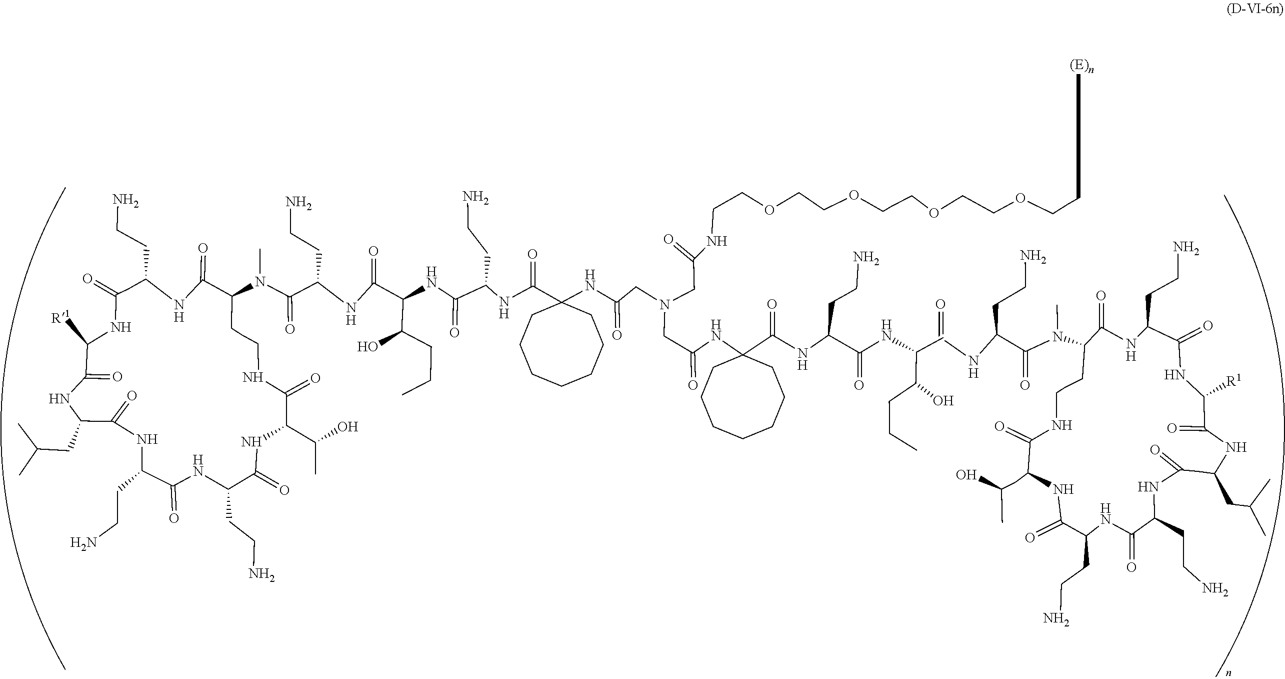

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-7):

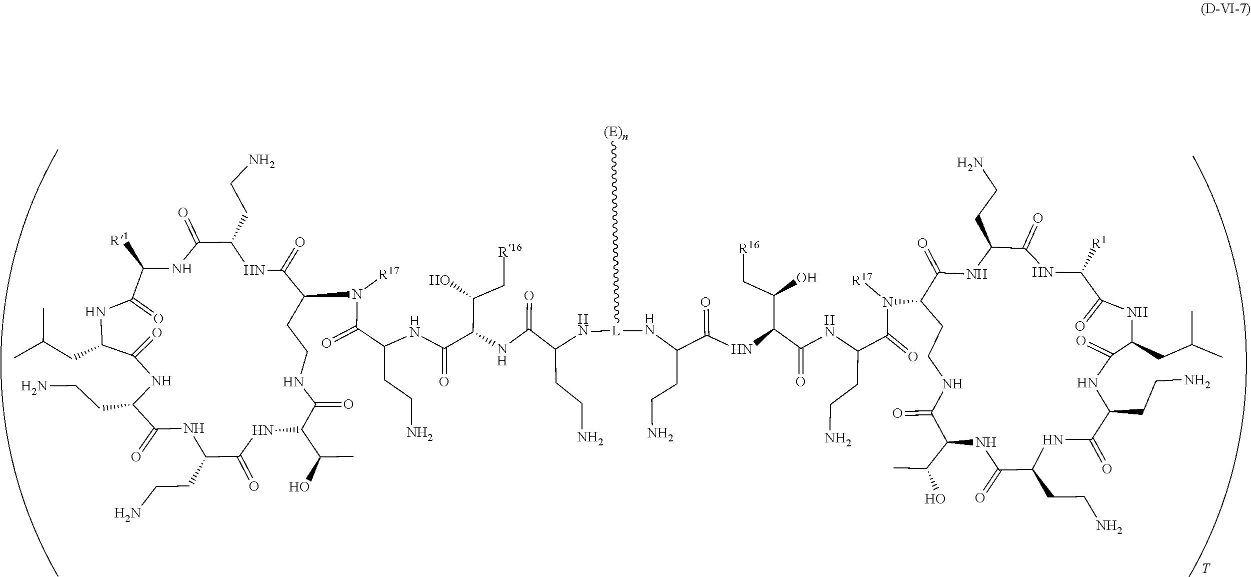

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R′¹⁶ and R¹⁶ is, independently, a lipophilic moiety, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments, the conjugate is described by formula (D-VI-8):

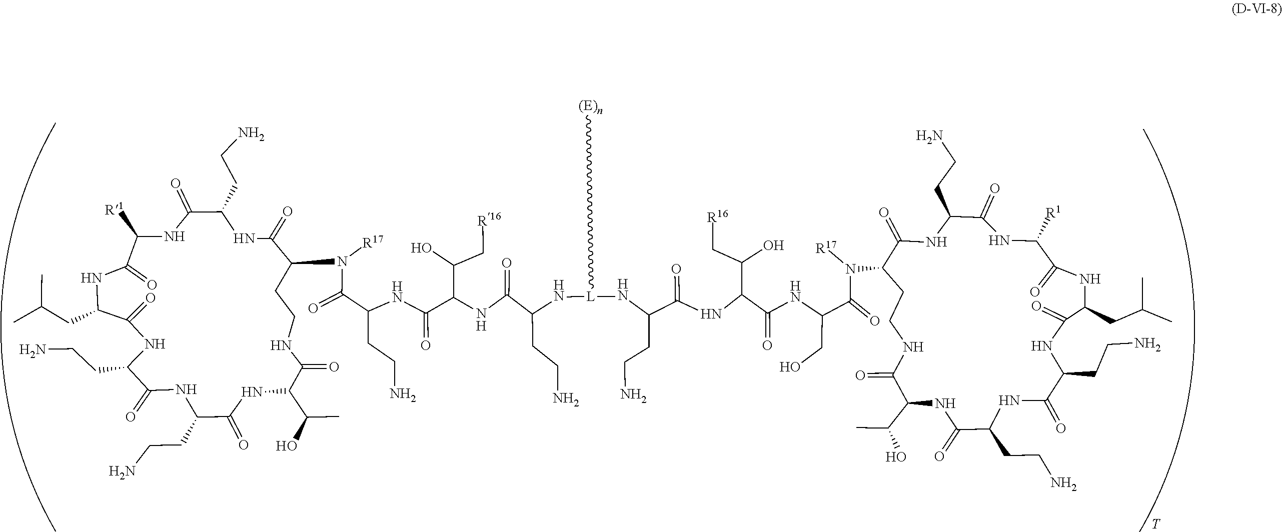

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R′¹⁶ and R¹⁶ is, independently, a lipophilic moiety, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments, the conjugate is described by formula (D-VI-9):

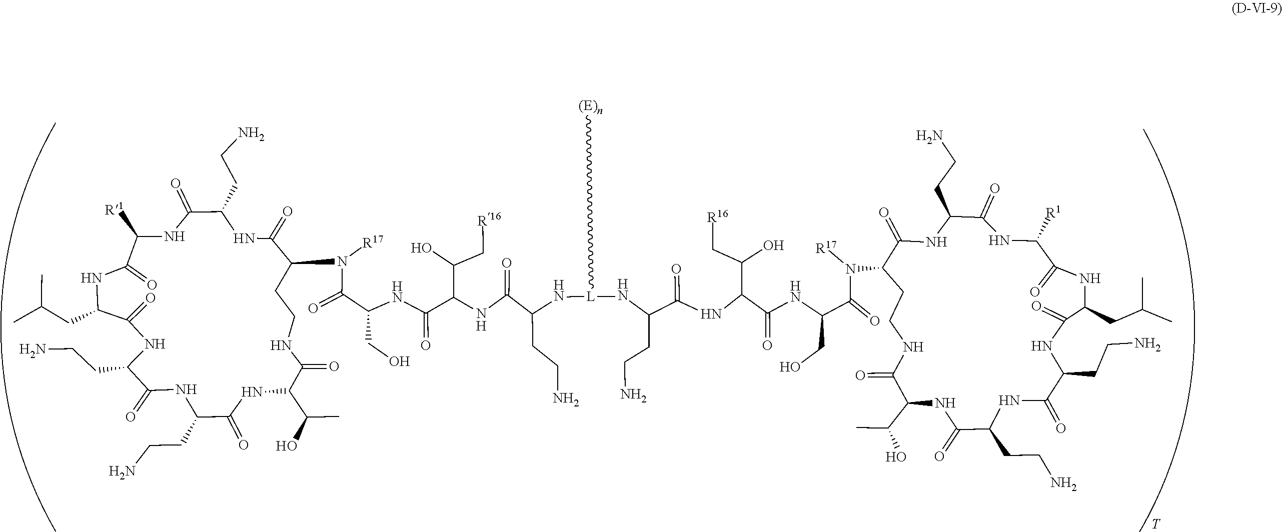

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R′¹⁶ and R¹⁶ is, independently, a lipophilic moiety, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments, the conjugate is described by formula (D-VI-10):

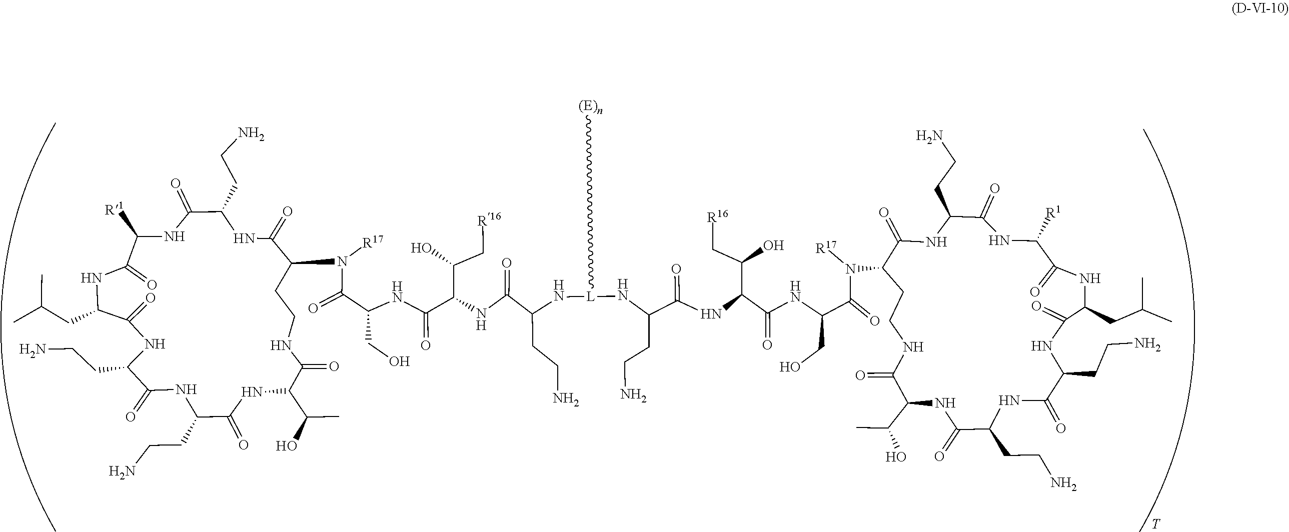

wherein each of R¹ and R′¹ is, independently, optionally substituted benzyl. CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R′¹⁶ and R¹⁶ is, independently, a lipophilic moiety, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments, each of R′¹⁶ and R¹⁶ is, independently, C1-C6 alkyl or benzyl.

In some embodiments, the conjugate is described by formula (D-VI-11):

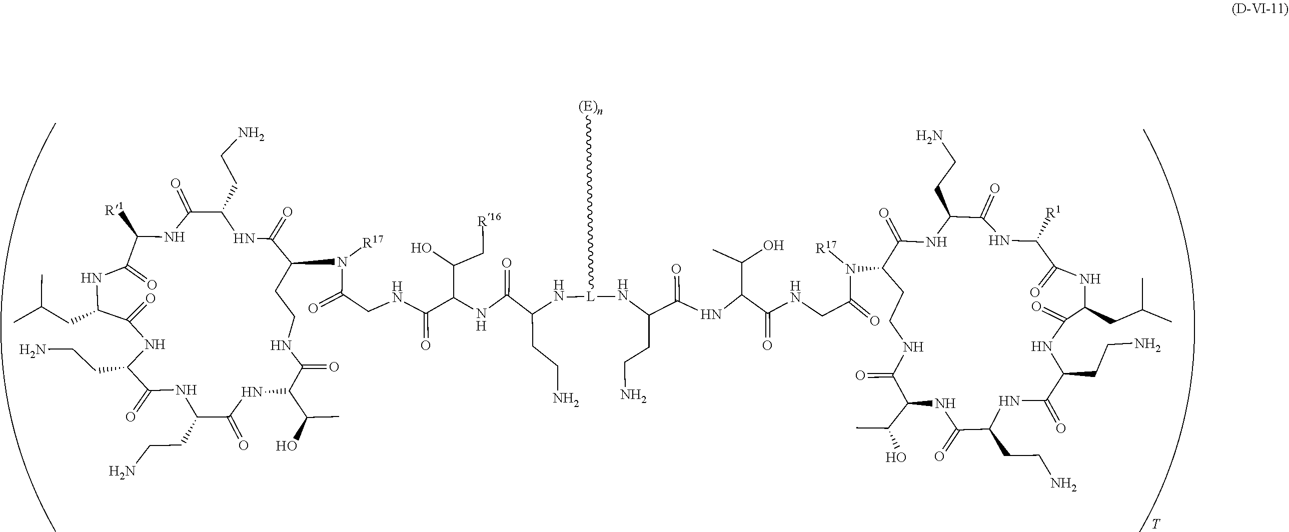

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-12):

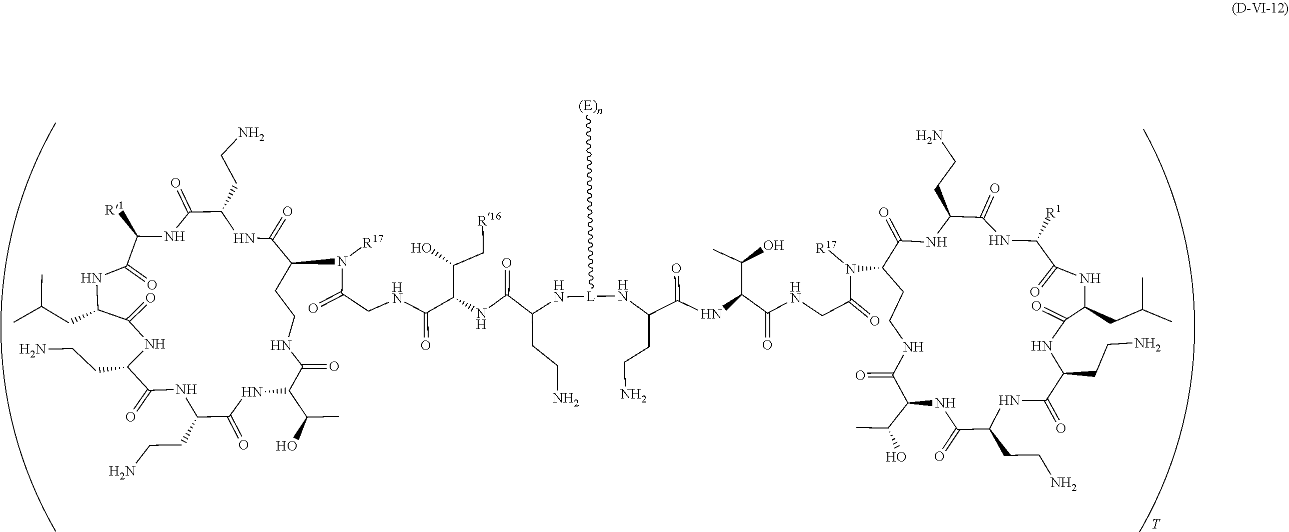

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-12a):

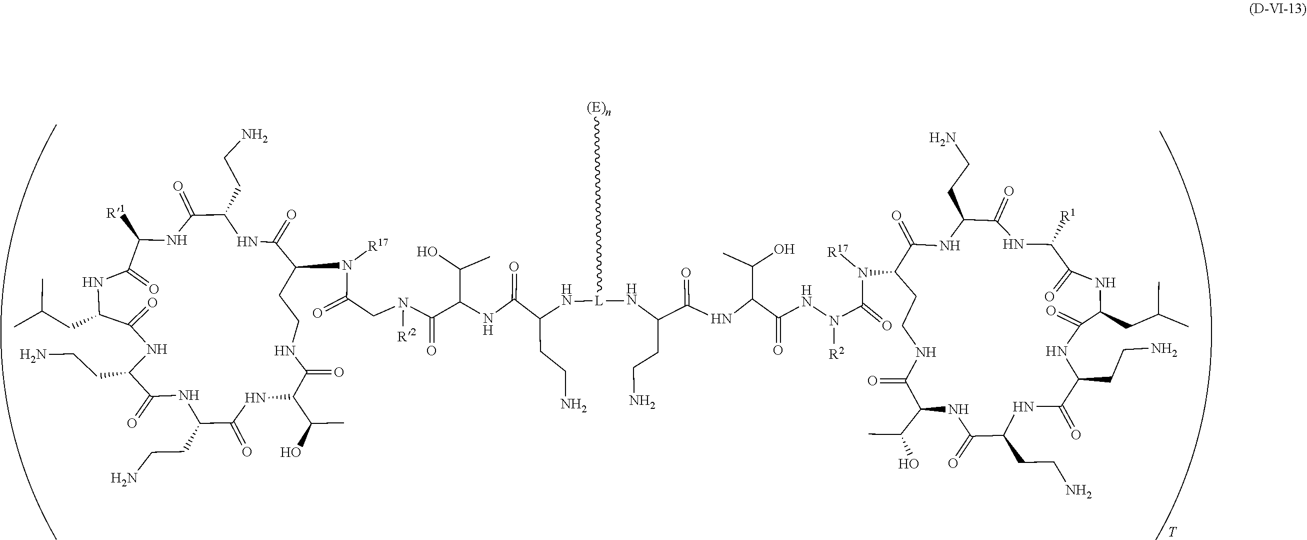

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

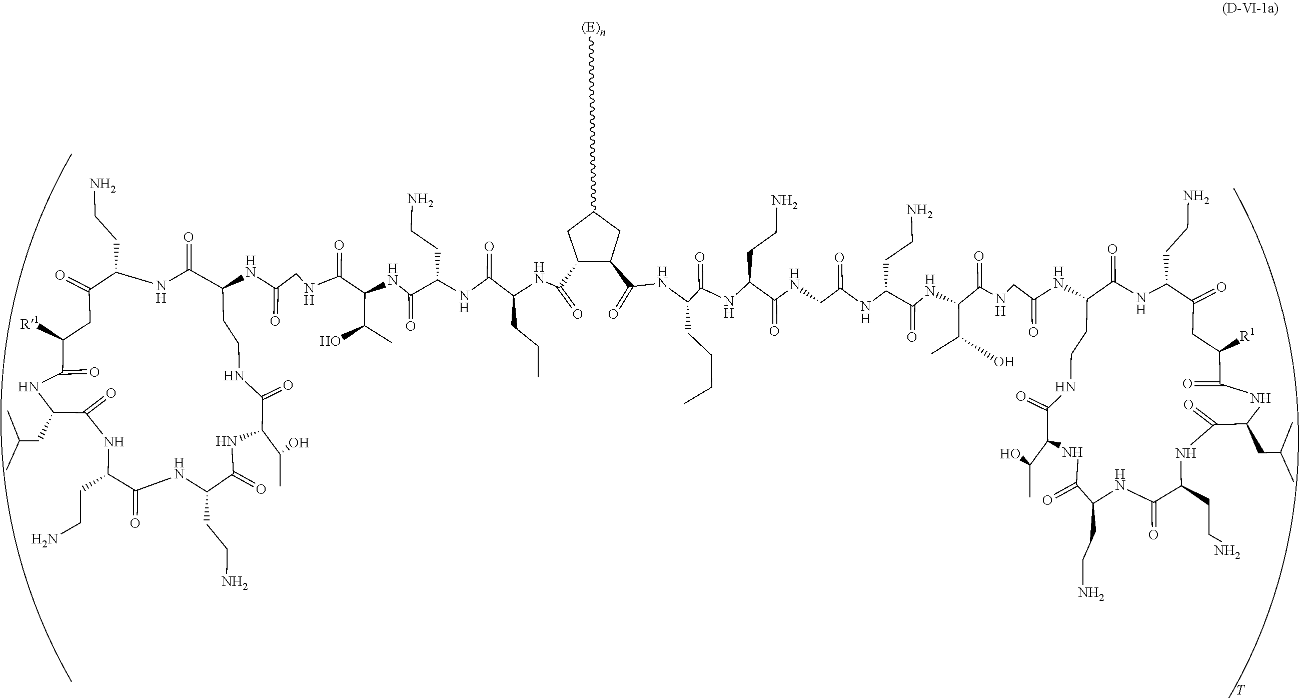

wherein each of R¹ and R′¹ is, independently, optionally substituted benzyl, CH₂CH(CH₃)₂ cyclopropylmethyl, cyclopentylmethyl, cyclohexylmethyl, naphthylmethyl, optionally substituted thiophenylmethyl, or optionally substituted furanylmethyl; and each of R′² and R² is, independently, H, C1-C8 alkyl, or C2-C8 alkamino; or a pharmaceutically acceptable salt thereof.

In some embodiments, the conjugate is described by formula (D-VI-14):

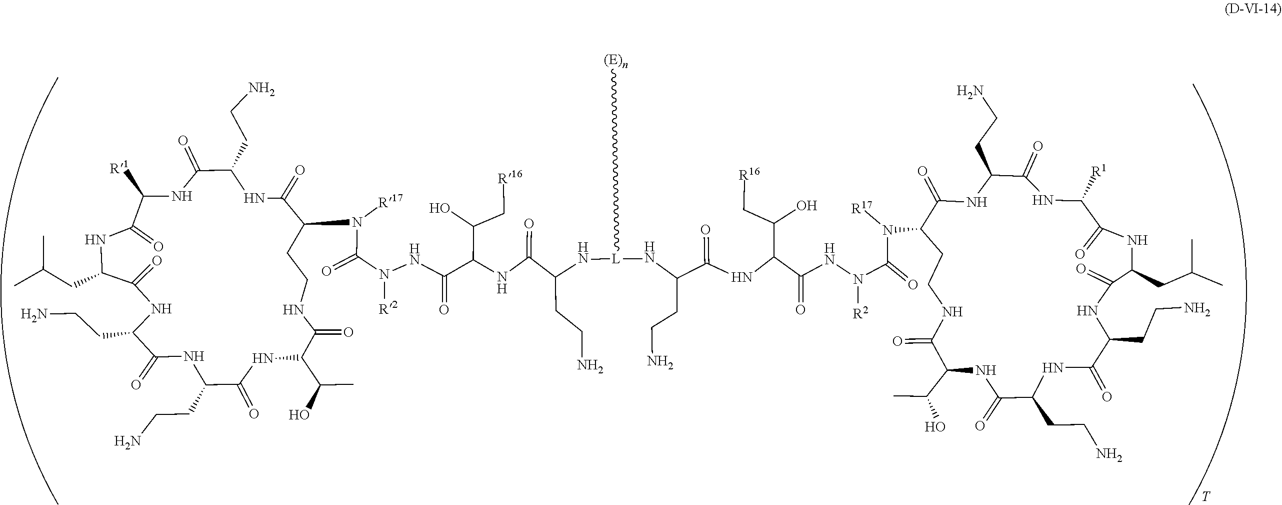

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R′² and R² is, independently, H, C1-C8 alkyl, or C2-C8 alkamino; and each of R′¹⁶ and R¹⁶ is, independently, a lipophilic moiety, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; or a pharmaceutically acceptable salt thereof.

In some embodiments, each of R′¹⁶ and R¹⁶ is, independently, C1-C6 alkyl, benzyl, or phenethyl.

In some embodiments, the conjugate is described by formula (D-VI-14a):

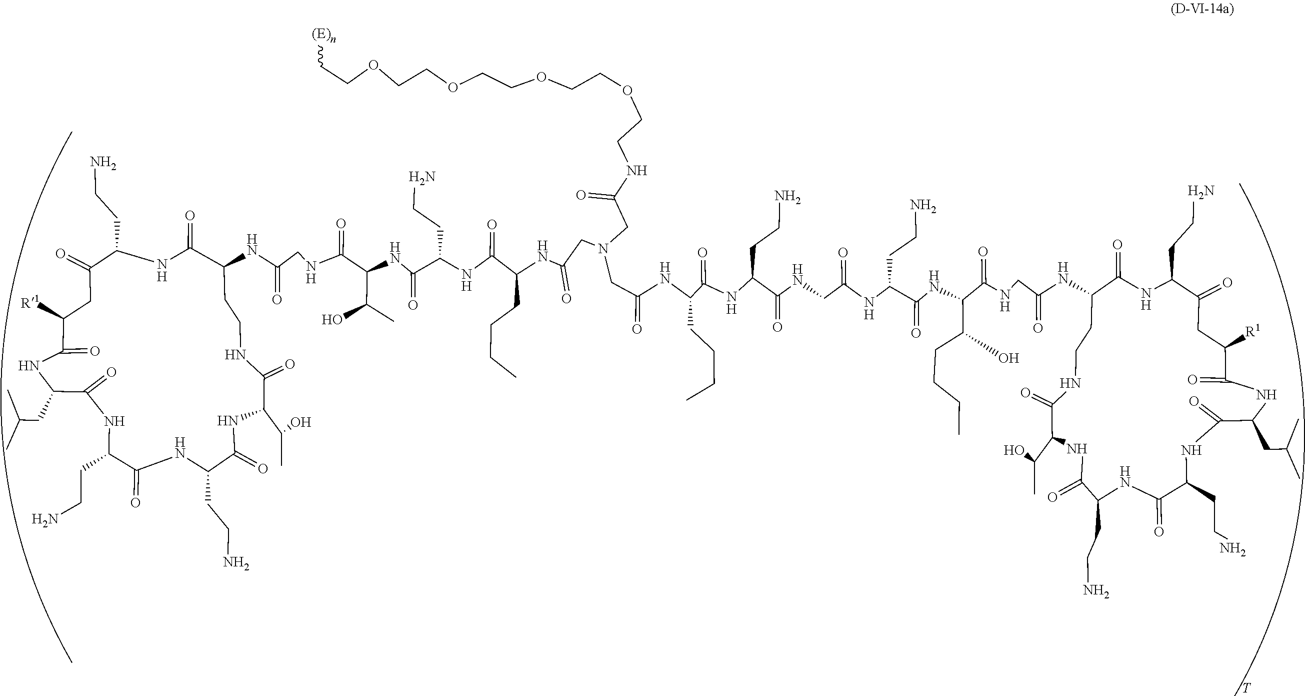

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-15):

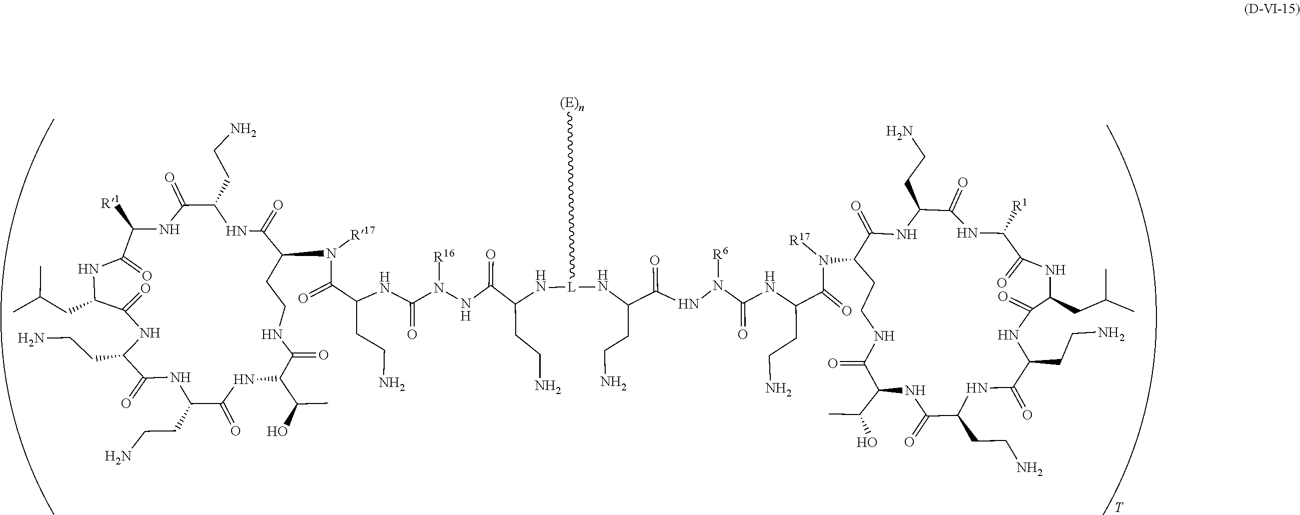

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; and each of R′⁶ and R⁶ is, independently, H, C1-C8 alkyl, or C2-C8 alkamino; or a pharmaceutically acceptable salt thereof.

In some embodiments, the conjugate is described by the formula (D-VI-15a):

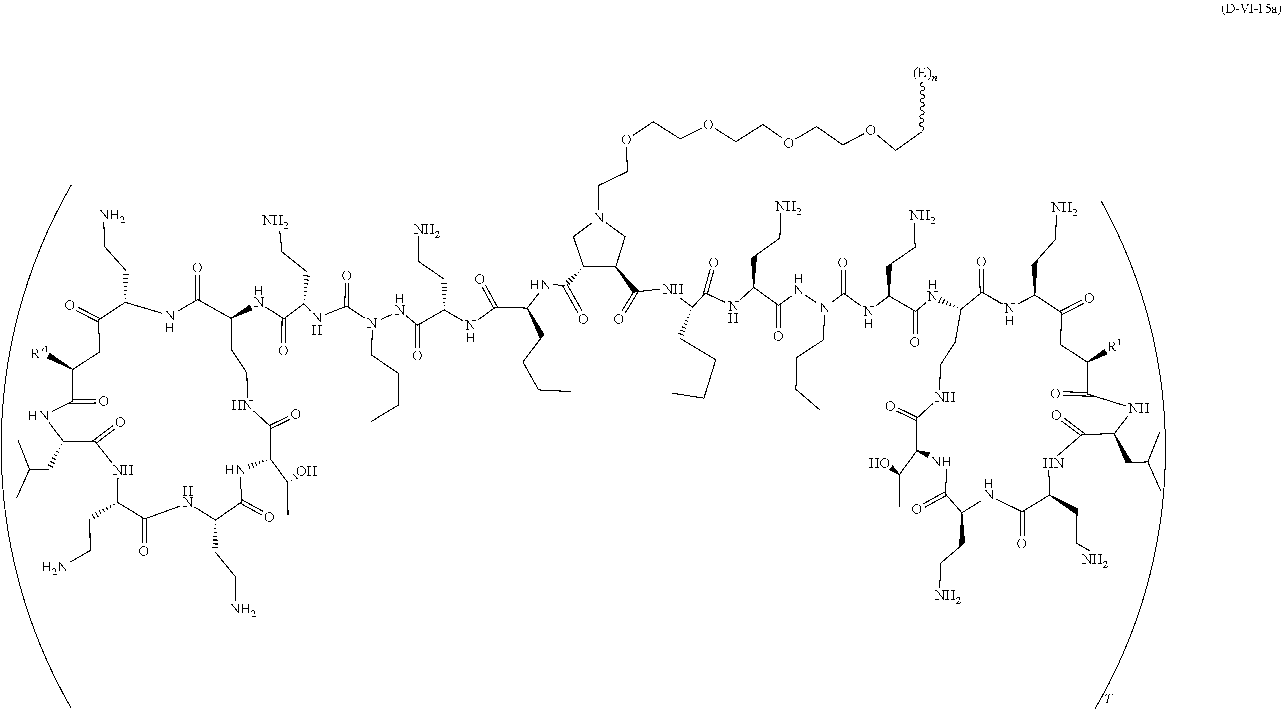

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-VI-16):

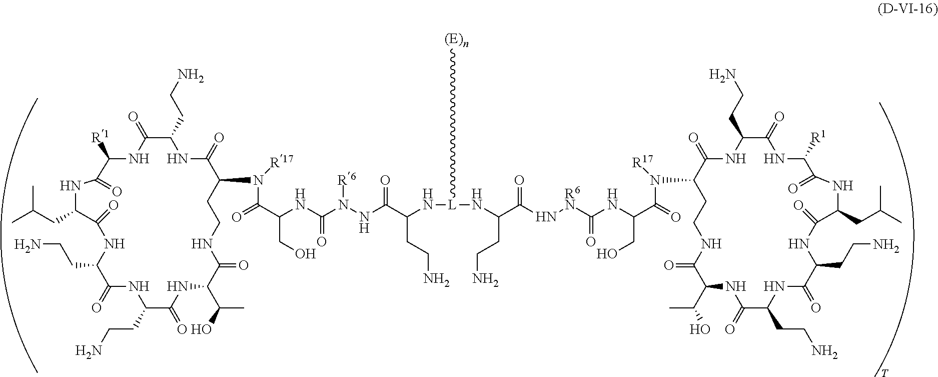

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; and each of R′⁶ and R⁶ is, independently, H. C1-C8 alkyl, or C2-C8 alkamino; or a pharmaceutically acceptable salt thereof.

In some embodiments of this aspect, the conjugate is described by formula (D-VII):

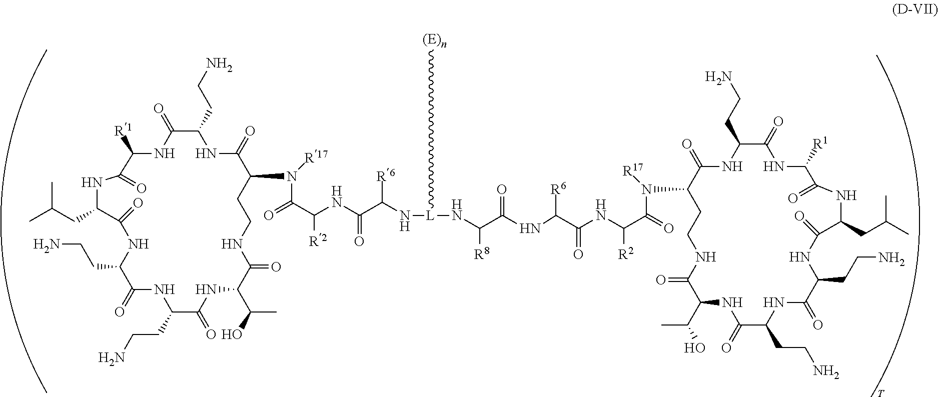

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R², R⁶, R⁸, R′², and R′⁶ is, independently, a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments of this aspect, the conjugate is described by formula (D-VIII):

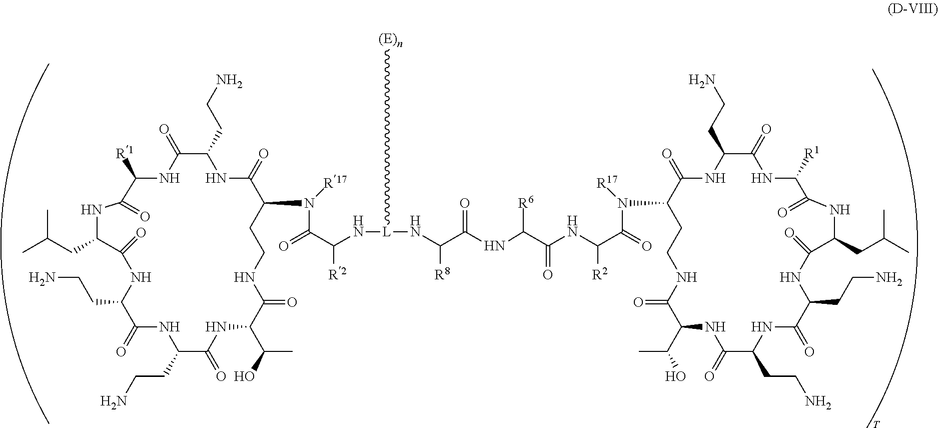

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R², R⁶, R⁸, and R′² is, independently, a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments of this aspect, the conjugate is described by formula (D-IX):

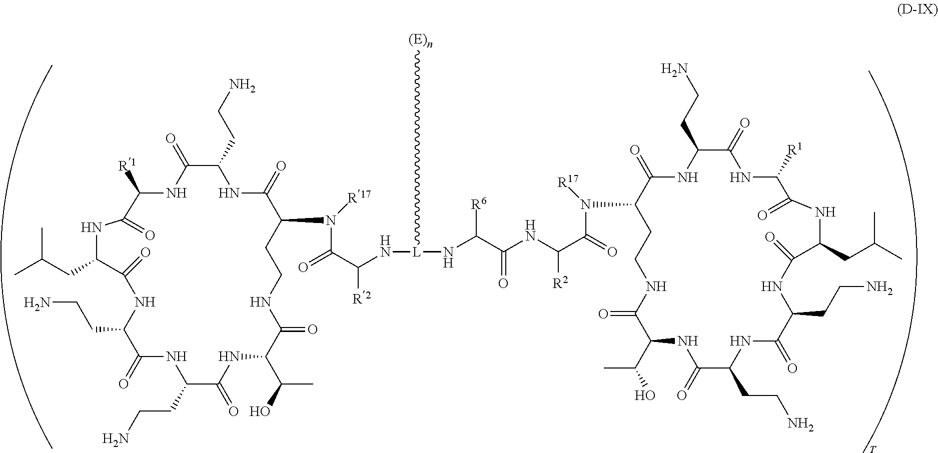

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R², R⁶, and R′² is, independently, a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments, the conjugate is described by formula (D-IX-1):

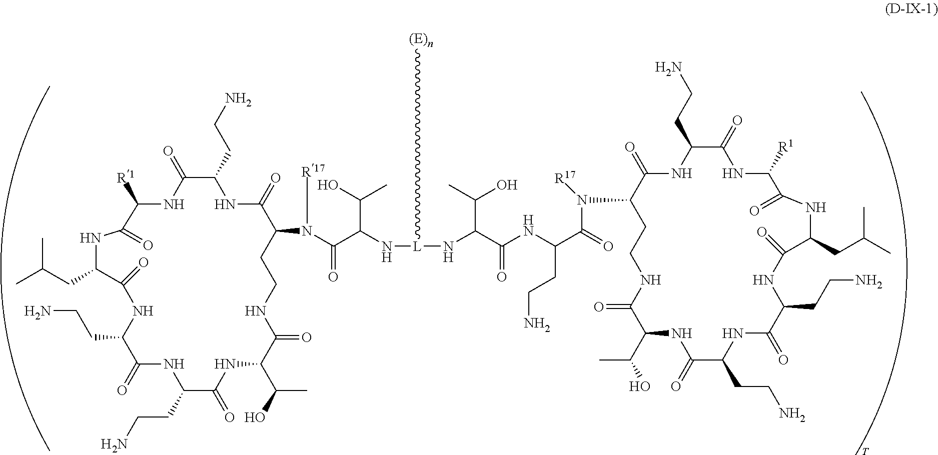

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-IX-2):

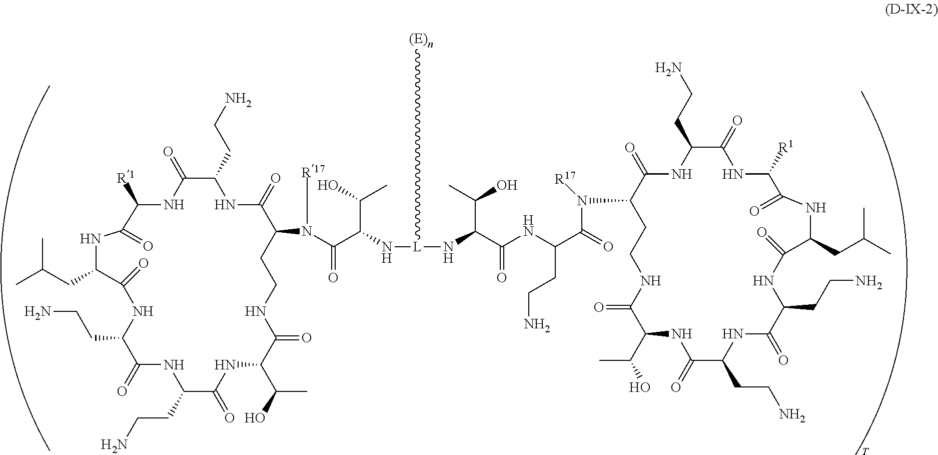

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In some embodiments, the conjugate is described by formula (D-IX-3):

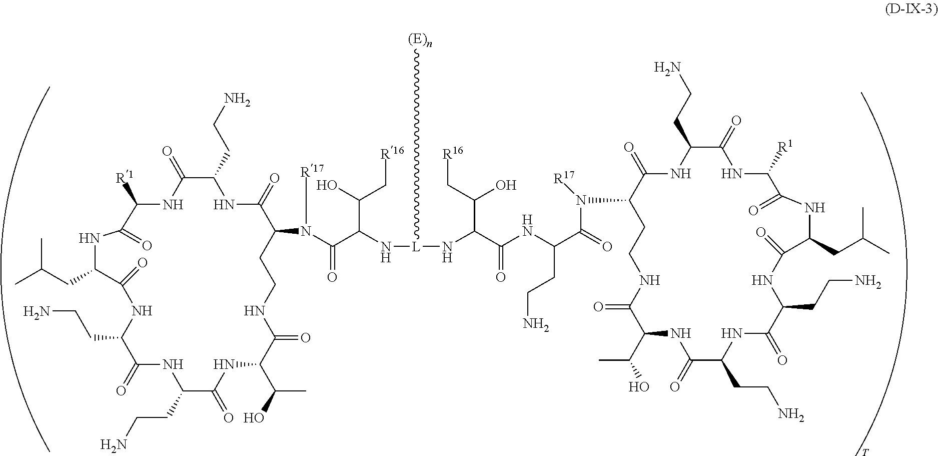

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R′¹⁶ and R¹⁶ is, independently, a lipophilic moiety, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments, the conjugate is described by formula (D-IX-4):

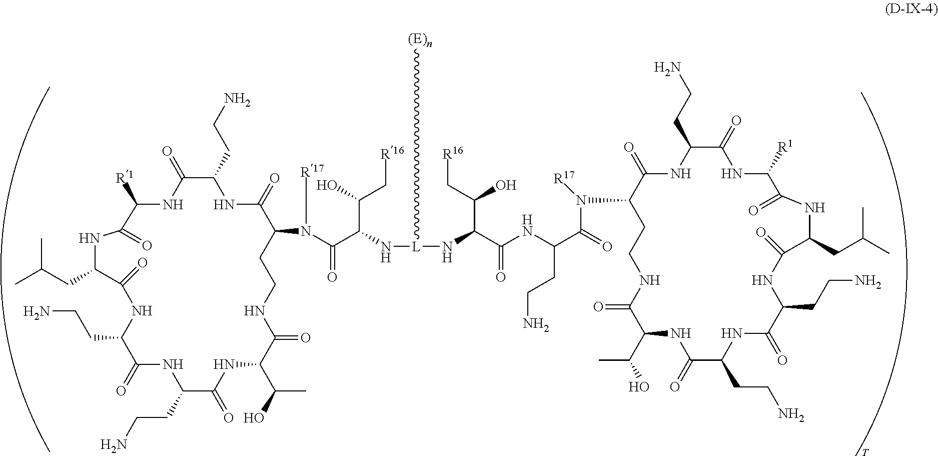

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R′¹⁶ and R¹⁶ is, independently, a lipophilic moiety, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments, each of R′¹⁶ and R¹⁶ is, independently, C1-C6 alkyl or benzyl.

In some embodiments of this aspect, the conjugate is described by formula (D-X):

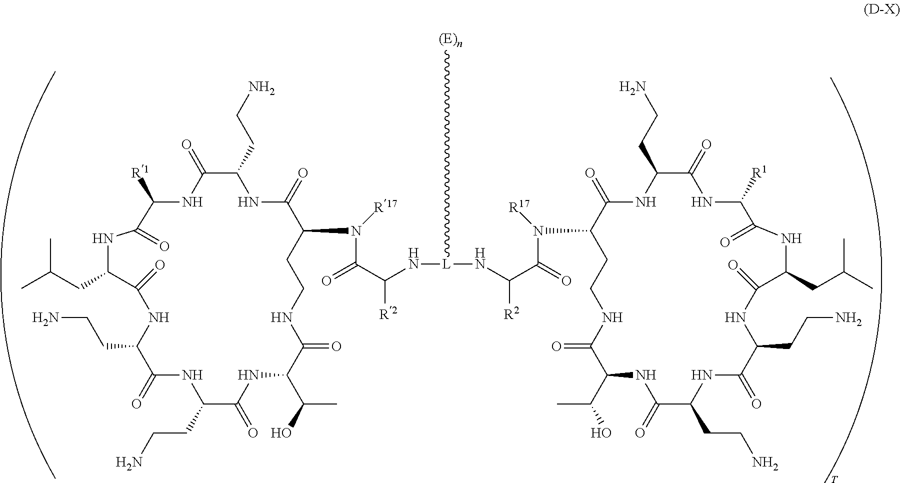

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R² and R′² is, independently, a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments of this aspect, the conjugate is described by formula (D-XI):

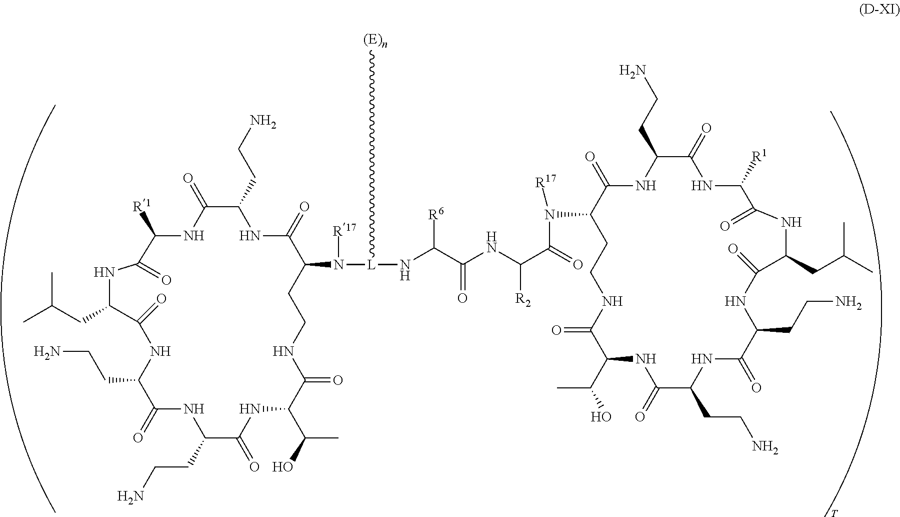

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R² and R⁶ is, independently, a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments, the conjugate is described by formula (D-XI-1):

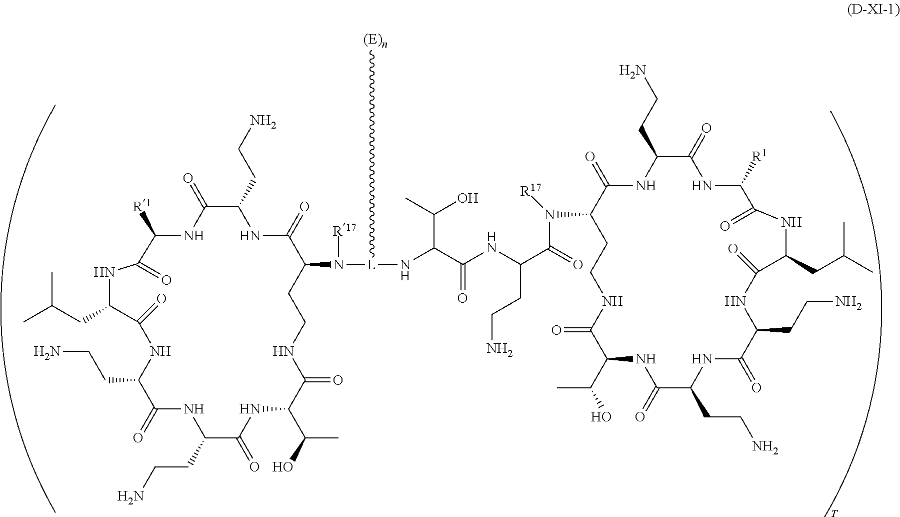

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-XI-2):

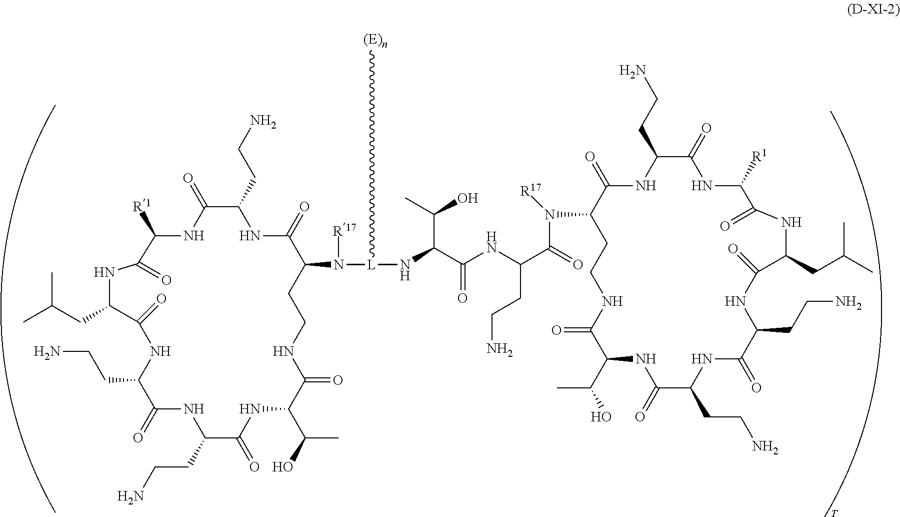

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-XI-3):

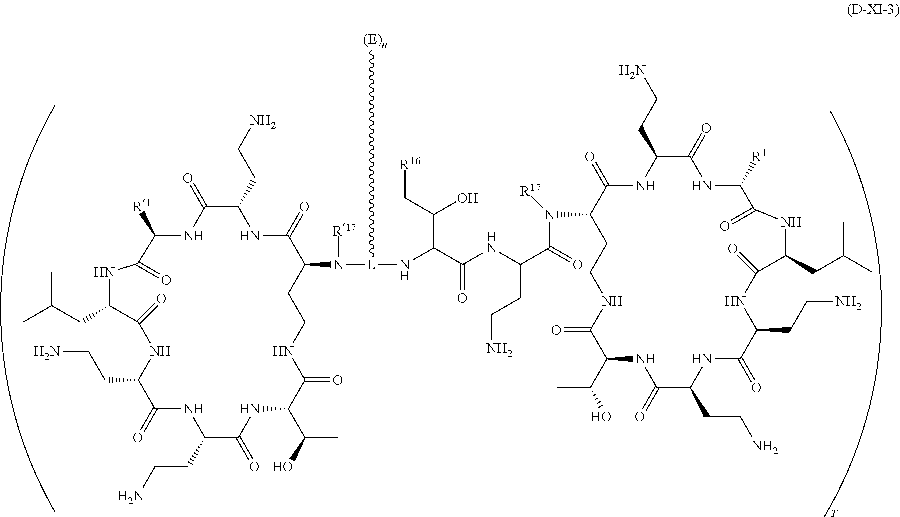

wherein each of R¹ and R′¹ is, independently, optionally substituted benzyl. CH₂CH(CH₃)₂, cyclopropylmethyl, cyclopentylmethyl, cyclohexylmethyl, naphthylmethyl, optionally substituted thiophenylmethyl, or optionally substituted furanylmethyl; R¹⁶ is a lipophilic moiety, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments, the conjugate is described by formula (D-XI-4):

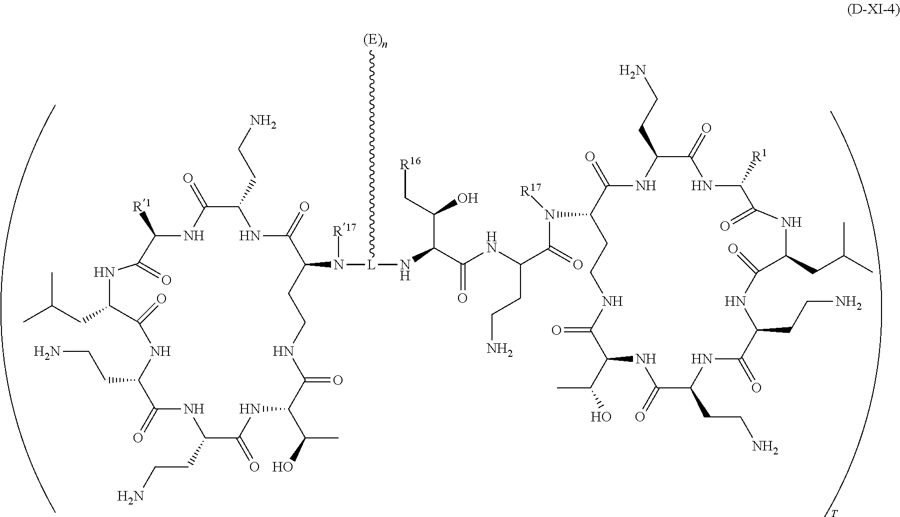

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; R¹⁶ is a lipophilic moiety, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments, R¹⁶ is C1-C6 alkyl or benzyl.

In some embodiments of this aspect, the conjugate is described by formula (D-XII):

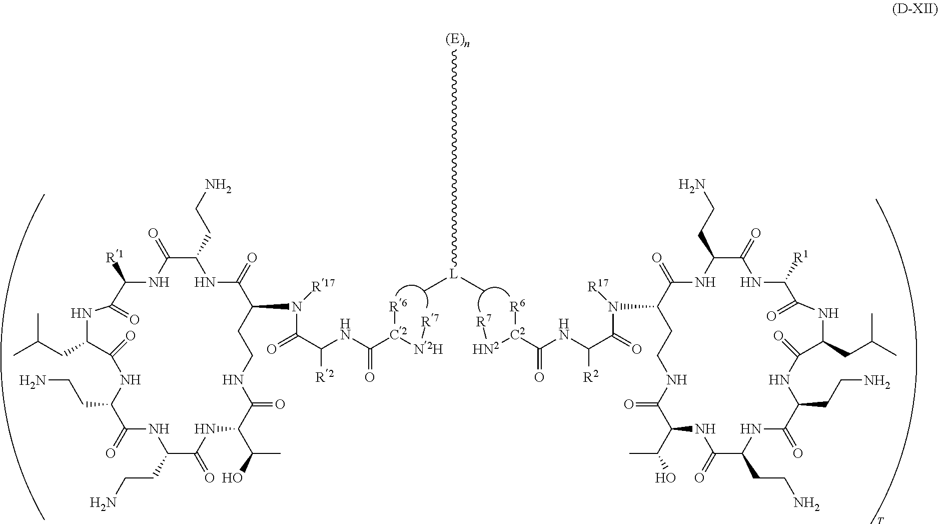

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R² and R′² is, independently, a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; R⁶, R⁷, N², and C² together form an optionally substituted 5-8 membered ring including optionally substituted C3-C7 heterocycloalkyl including an N heteroatom and additional 0-2 heteroatoms independently selected from N, O, and S, or optionally substituted C2-C7 heteroaryl including an N heteroatom and additional 0-2 heteroatoms independently selected from N, O, and S; R′⁶, R′⁷, N′², and C′² together form an optionally substituted 5-8 membered ring including optionally substituted C3-C7 heterocycloalkyl including an N heteroatom and additional 0-2 heteroatoms independently selected from N, O, and S, or optionally substituted C2-C7 heteroaryl including an N heteroatom and additional 0-2 heteroatoms independently selected from N, O, and S; or a pharmaceutically acceptable salt thereof.

In some embodiments, the conjugate is described by formula (D-XIIa):

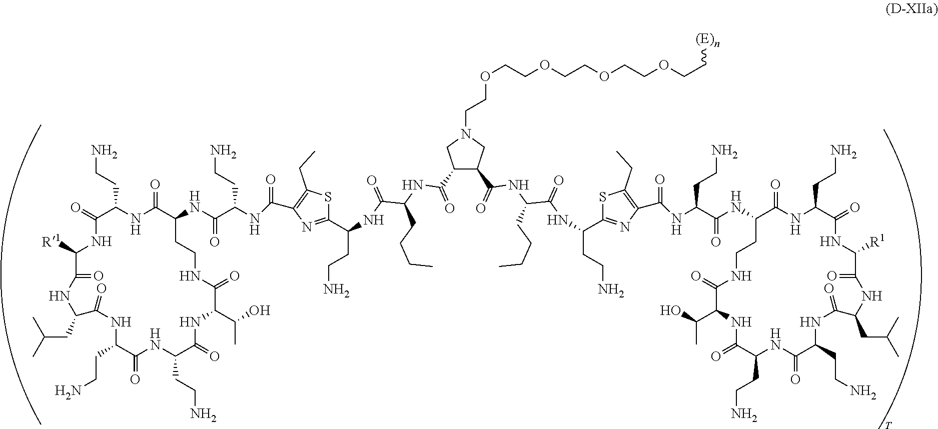

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (D-XII-1):

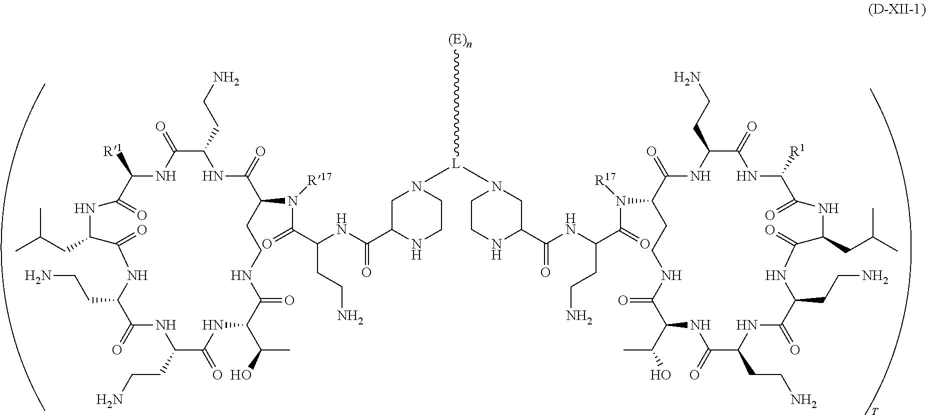

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments of this aspect, the conjugate is described by formula (D-XIII):

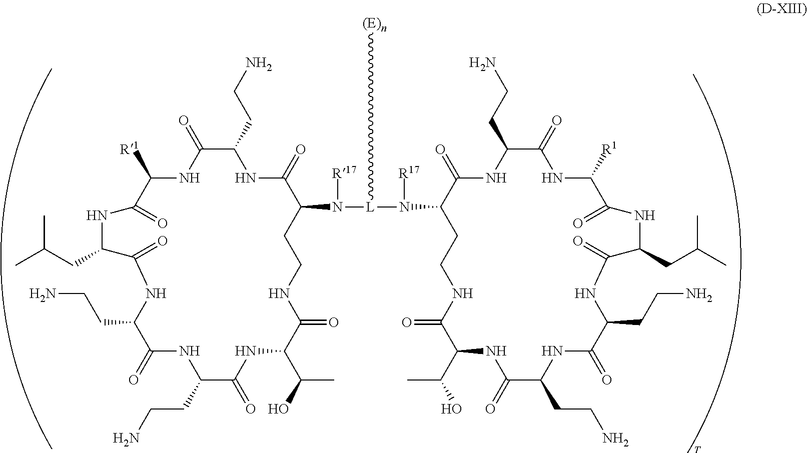

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In some embodiments, the conjugate is described by formula (D-XIIIa):

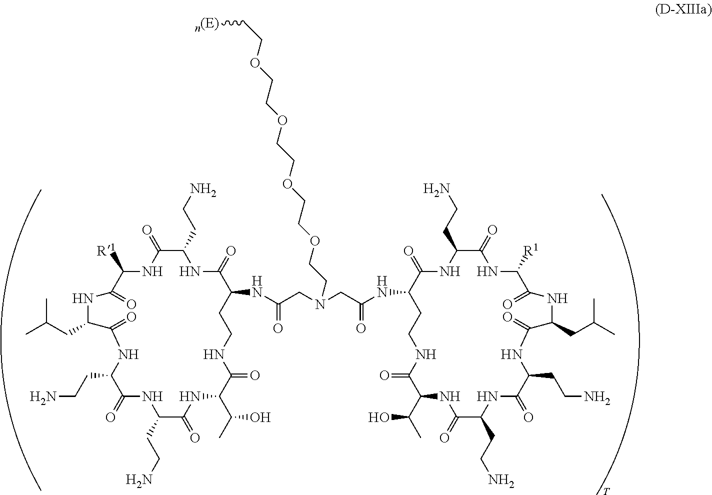

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In some embodiments, R² is optionally substituted C1-C5 alkamino. In some embodiments, R′² is optionally substituted C1-C5 alkamino (e.g., CH₂NH₂ or CH₂CH₂NH₂).

In some embodiments, R² is a polar moiety. In some embodiments, R′² is a polar moiety. In some embodiments, R⁶ is a polar moiety. In some embodiments, R′⁶ is a polar moiety. In some embodiments, the polar moiety includes a hydroxyl group, a carboxylic acid group, an ester group, or an amide group. In some embodiments, the polar moiety is hydroxyl substituted C1-C4 alkyl. In some embodiments, the polar moiety is CHCH₃OH or CH₂OH.

In some embodiments, R⁸ is optionally substituted C1-C5 alkamino. In some embodiments, R′⁸ is optionally substituted C1-C5 alkamino (e.g., CH₂NH₂ or CH₂CH₂NH₂). In some embodiments, R⁸ is optionally substituted C5-C15 aryl. In some embodiments, R′⁸ is optionally substituted C5-C15 aryl. In some embodiments, the optionally substituted C5-C15 aryl is naphthyl.

In some embodiments, R⁸, R⁷, N², and C² together form a 5- or 6-membered ring including C4-C5 heterocycloalkyl including an N heteroatom and additional 0 or 1 heteroatom independently selected from N, O, and S; and wherein R′⁶, R′⁷, N′², and C′² together form a 5- or 6-membered ring including C4-C5 heterocycloalkyl including an N heteroatom and additional 0 or 1 heteroatom independently selected from N, O, and S.

In some embodiments, each of R¹⁷ and R′¹⁷ is H. In some embodiments, each of R¹⁷ and R′¹⁷ is methyl.

In some embodiments, each of R¹ and R′¹ is, independently, benzyl or CH₂CH(CH₃)₂. In some embodiments, R¹ and R′¹ are each benzyl. In some embodiments, R¹ and R′¹ are each CH₂CH(CH₃)₂.

In some embodiments, L′, L, or L¹ includes one or more optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, optionally substituted C2-C15 heteroarylene, O, S, NRⁱ, P, carbonyl, thiocarbonyl, sulfonyl, phosphate, phosphoryl, or imino, wherein Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C3-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl.

In some embodiments, the backbone of L′, L, or L¹ consists of one or more optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, optionally substituted C2-C15 heteroarylene, O, S, NRⁱ, P, carbonyl, thiocarbonyl, sulfonyl, phosphate, phosphoryl, or imino, wherein Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C3-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl.

In some embodiments, L′, L, or L′¹ is oxo substituted. In some embodiments, the backbone of L′, L, or L¹ includes no more than 250 atoms. In some embodiments, L′, L, or L¹ is capable of forming an amide, a carbamate, a sulfonyl, or a urea linkage. In some embodiments, L or L¹ is a bond.

In some embodiments, each L is described by formula (D-L-I):

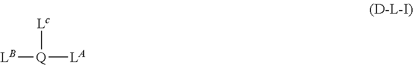

wherein L^{A} is described by formula G^{A1}-(Z^{A1})_{g1}-(Y^{A1})ₕ₁-(Z^{A2})ᵢ₁-(Y^{A2})-(Z^{A3})ₗ₁-(Y^{A4})ₗ₁-(Z^{A4})ₘ₁-(Y^{A4})ₙ₁-(Z^{A5})_{O1}-G^{A2}; L^{B} is described by formula G^{B1}-(Z^{B1})_{g2}-(Y^{B1})ₕ₂-(Z^{B2})ᵢ₂-(Y^{B2})ⱼ₂-(Z^{B3})ₖ₂-(Y^{B3})ₗ₂-(Z^{B4})ₘ₂-(Y^{B4})ₙ₂-(Z^{B5})_{O2}-G^{B2}; L^{C} is described by formula G^{C1}-(Z^{C1})_{g3}-(Y^{C1})ₕ₃-(Z^{C2})ᵢ₃-(Y^{C2})ⱼ₃-(Z^{C3})ₖ₃-(Y^{C3})ₗ₃-(Z^{C4})ₘ₃-(Y^{C4})ₙ₃-(Z^{C5})_{O}_{₃}-G^{C2}; G^{A1} is a bond attached to Q in formula (L-I); G^{A2} is a bond attached to A1 or M1 if A1 is absent; G^{B1} is a bond attached to Q in formula (L-I); G^{B2} is a bond attached to A2 or M2 if A2 is absent; G^{C1} is a bond attached to Q in formula (L-I); G^{C2} is a bond attached to E; each of Z^{A1}, Z^{A2}, Z^{A3}, Z^{A4}, Z^{A5}, Z^{B1}, Z^{B2}, Z^{B3}, Z^{B4}, Z^{B5}, Z^{C1}, Z^{C2}, Z^{C3}, Z^{C4}, and Z^{C5} is, independently, optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene; each of Y^{A1}, Y^{A2}, Y^{A3}, Y^{A4}, Y^{B1}, Y^{B2}, Y^{B3}, Y^{B4}, Y^{C1}, Y^{C2}, Y^{C3}, and Y^{C4} is, independently, O, S, NRⁱ, P, carbonyl, thiocarbonyl, sulfonyl, phosphate, phosphoryl, or imino; Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C3-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl; each of g1, h1, i1, j1, k1, l1, m1, n1, o1, g2, h2, i2, j2, k2, l2, m2, n2, o2, g3, h3, i3, j3, k3, l3, m3, n3, and 03 is, independently, 0 or 1; Q is a nitrogen atom, optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene.

In some embodiments, depending on the structure and chemical formula of L^{C}, L^{C} may have two points of attachment to the Fc domain (e.g., two G^{C2}).

In some embodiments, L is

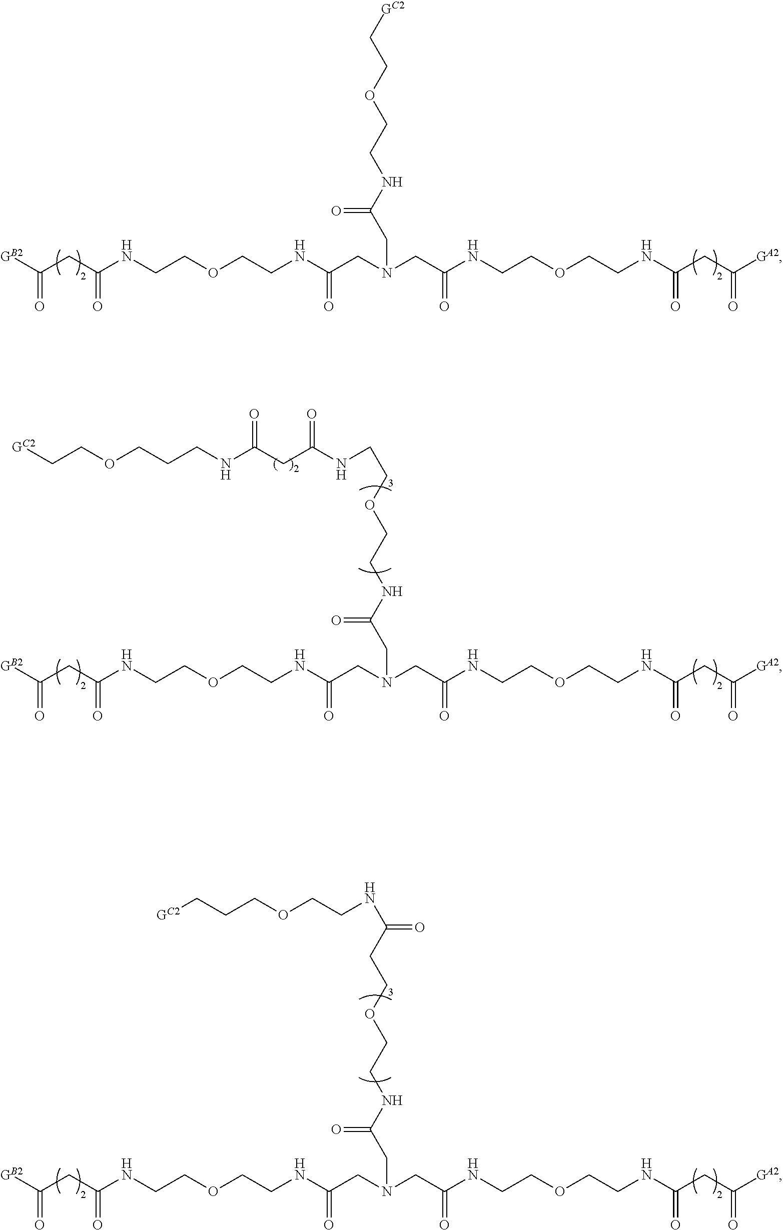

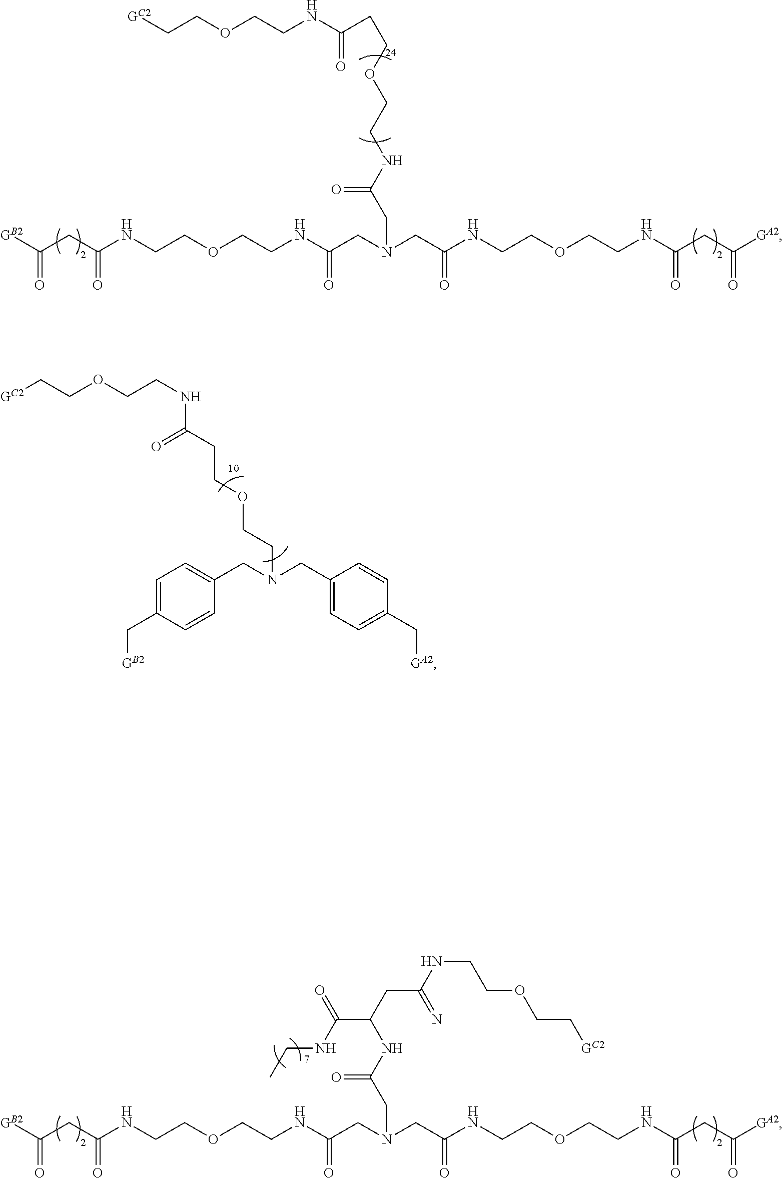

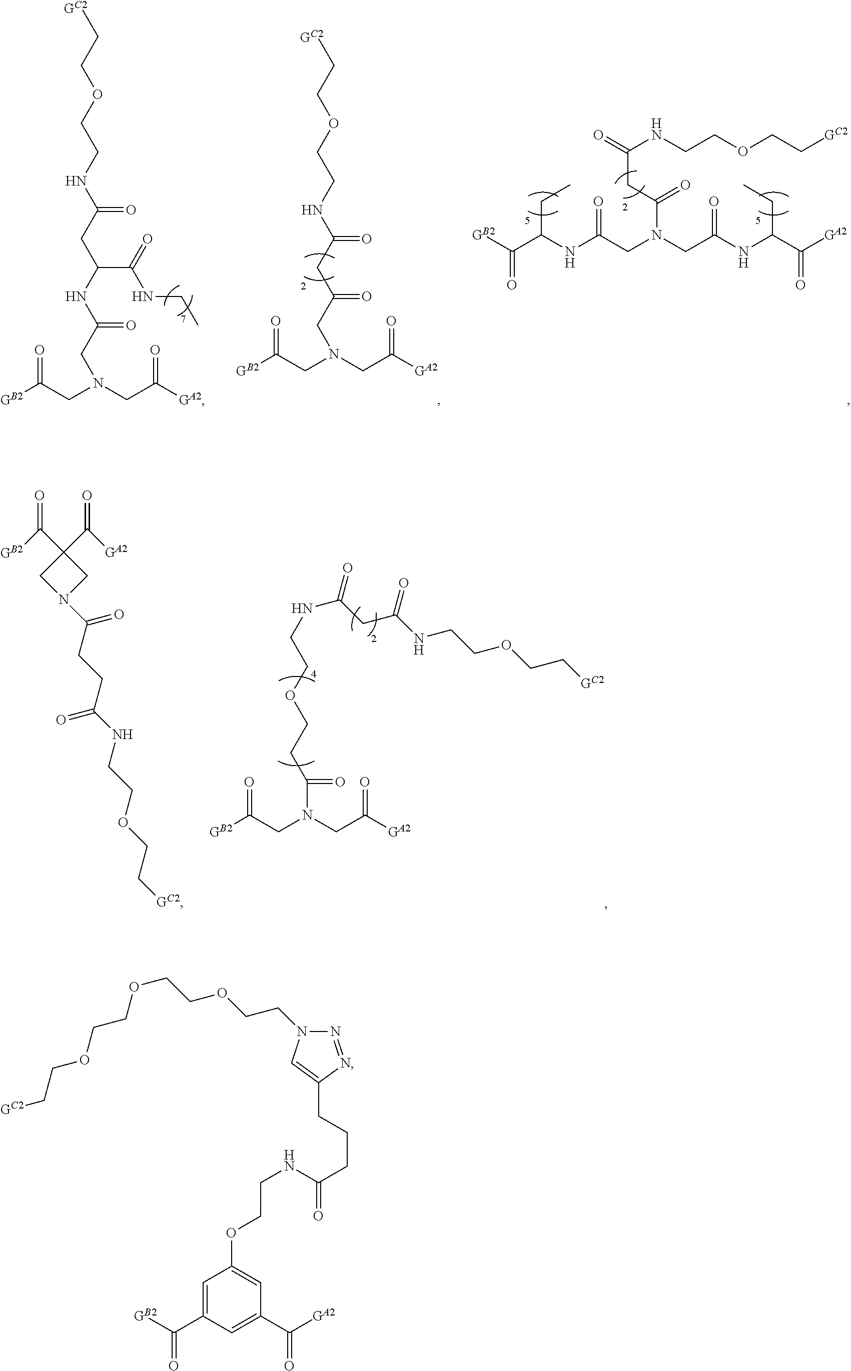

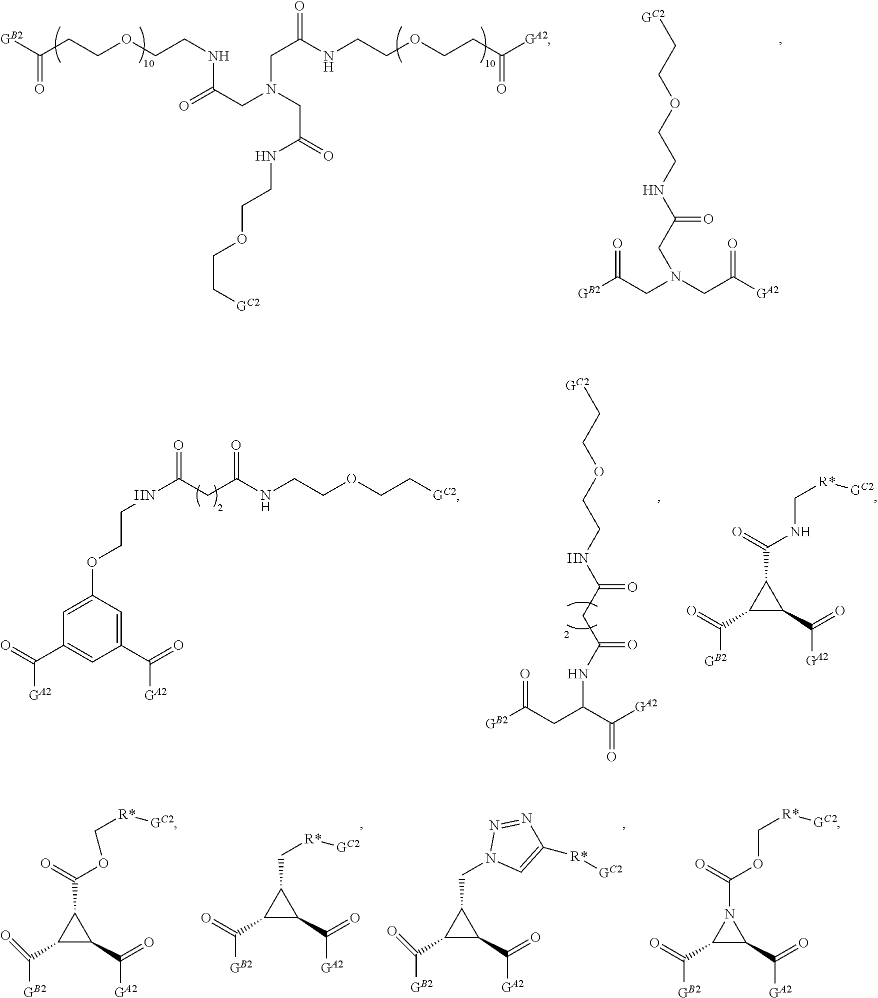

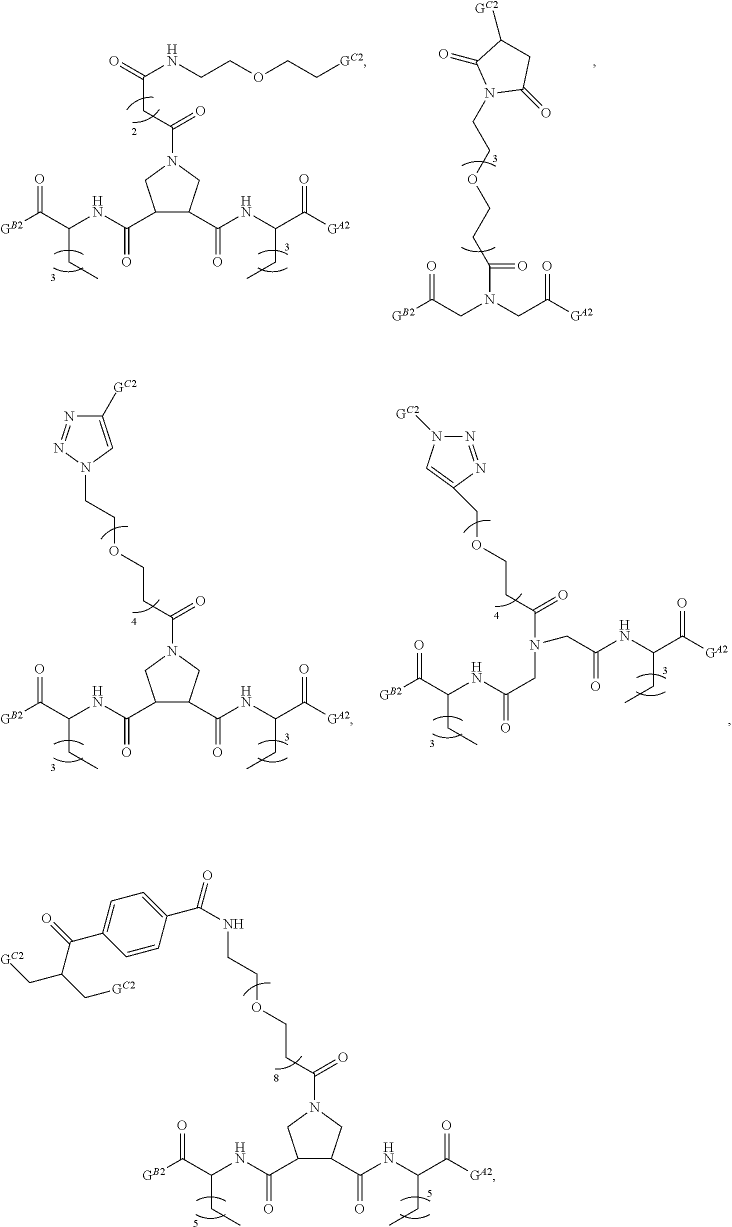

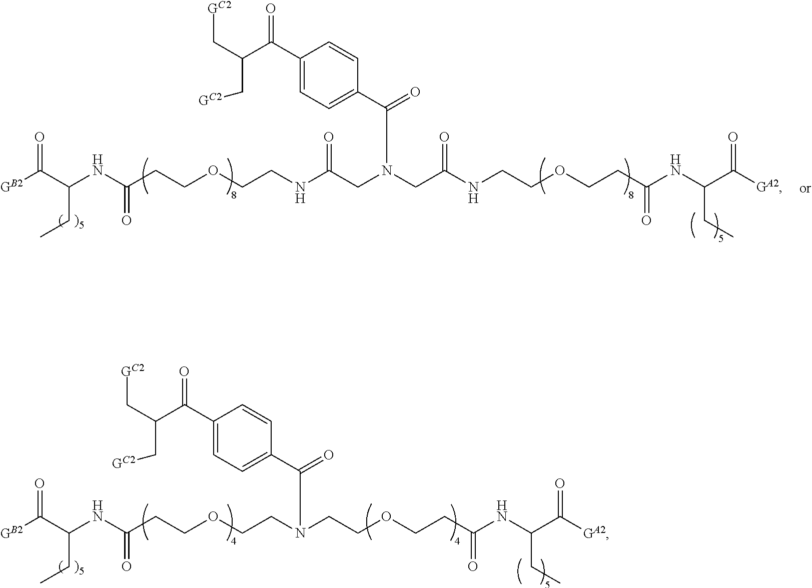

wherein R* is a bond or includes one or more of optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, optionally substituted C2-C15 heteroarylene, O, S, NRⁱ, P, carbonyl, thiocarbonyl, sulfonyl, phosphate, and imino, and wherein Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C3-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl.

In some embodiments of this aspect, E has the sequence of any one of SEQ ID NOs: 1-14, 1-29, or 1-31. In some embodiments of this aspect, when n is 2, E dimerizes to form an Fc domain.

In some embodiments of this aspect, the conjugate is described by formula (D-XIV):

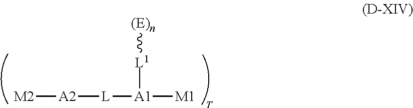

wherein each A1 is a 1-5 amino acid peptide covalently attached to the linking nitrogen in each M1; each A2 is a 1-5 amino acid peptide covalently attached to the linking nitrogen in each M2, or is absent; each of L and L¹ is a remainder of L′; or a pharmaceutically acceptable salt thereof.

In some embodiments, the conjugate is described by formula (D-XXIX)

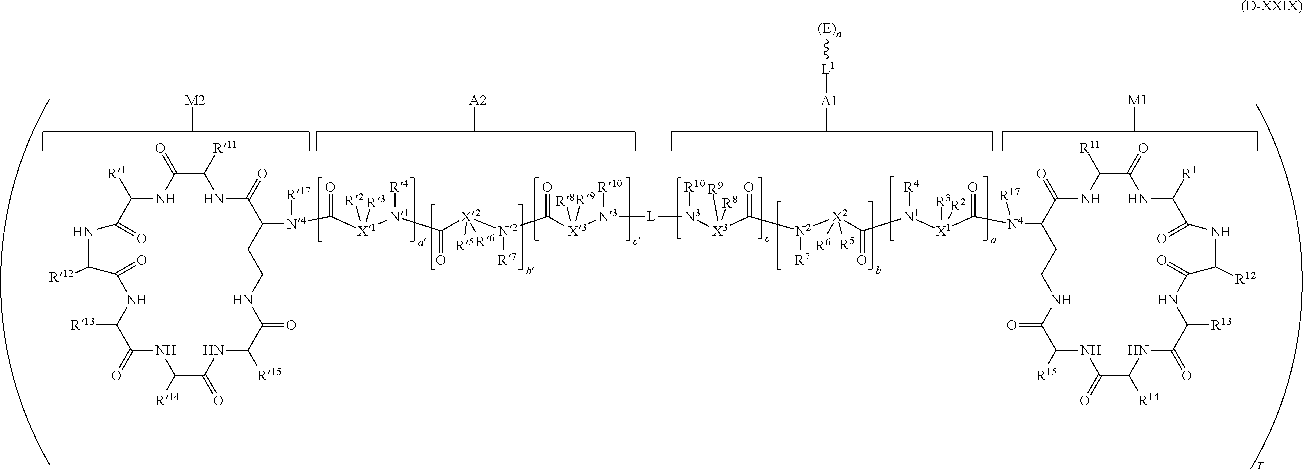

wherein each of L and L¹ is a remainder of L′; each of R¹, R¹², R′¹, and R′¹² is, independently, a lipophilic moiety, a polar moiety, or H; each of R¹¹, R¹³, R¹⁴, R′¹¹, R′¹³, and R′¹⁴ is, independently, optionally substituted C1-C5 alkamino, a polar moiety, a positively charged moiety, or H; each of R¹⁵ and R′¹⁵ is, independently, a lipophilic moiety or a polar moiety; each of R², R³, R⁴, R⁵, R⁶, R⁷, R⁸, R⁹, R¹⁰, R′², R′³, R′⁴, R′⁵, R′⁶, R′⁷, R′⁸, R′⁹, and R′¹⁰ is, independently, a lipophilic moiety, a positively charged moiety, a polar moiety, H, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; or two R groups on the same or adjacent atoms join to form an optionally substituted 3-8 membered ring (e.g., 5-8 membered ring); each or R¹⁷ and R′¹⁷ is, independently, H, C1-C3 alkyl, or C2-C3 alkamino; each of a′, b′, c′, b, and c is, independently, 0 or 1; each of N¹, N², N³, N⁴, N′¹, N′², N′³, and N′⁴ is a nitrogen atom; each of C¹, C³, C′¹, and C′³ is a carbon atom; and each of X¹, X², X³, X′¹, X′², and X′³ is, independently, a carbon atom or a nitrogen atom, wherein if X¹ is a nitrogen atom then R³ is absent, if X′¹ is a nitrogen atom then R′³ is absent, if X² is a nitrogen atom then R⁵ is absent, if X′² is a nitrogen atom then R′⁵ is absent, if X³ is a nitrogen atom then R⁹ is absent, and if X′³ is a nitrogen atom then R′⁹ is absent; or a pharmaceutically acceptable salt thereof.

In some embodiments, the conjugate includes at least one optionally substituted 3-8 membered ring (e.g., 5-8 membered ring) formed by joining (i) R², R³, and X¹; (ii) R³, R⁴, N¹, and X¹; (iii) R⁵, R⁶, and X²; (iv) R⁶, R⁷, N², and X²; (v) R⁸, R⁹, and X³; (vi) R⁹, R¹⁰, N³, and X³; (vii) R′², R′³, and X′¹; (viii) R′³, R′⁴, N′¹, and X′¹; (ix) R′⁵, R′⁶, and X′²; (x) R′⁶, R′⁷, N′², and X′²; (xi) R′⁸, R′⁹, and X′³; or (xii) R′⁹, R′¹⁰, N′³, and X′³.

In some embodiments, the conjugate is described by formula (D-XXIXa):

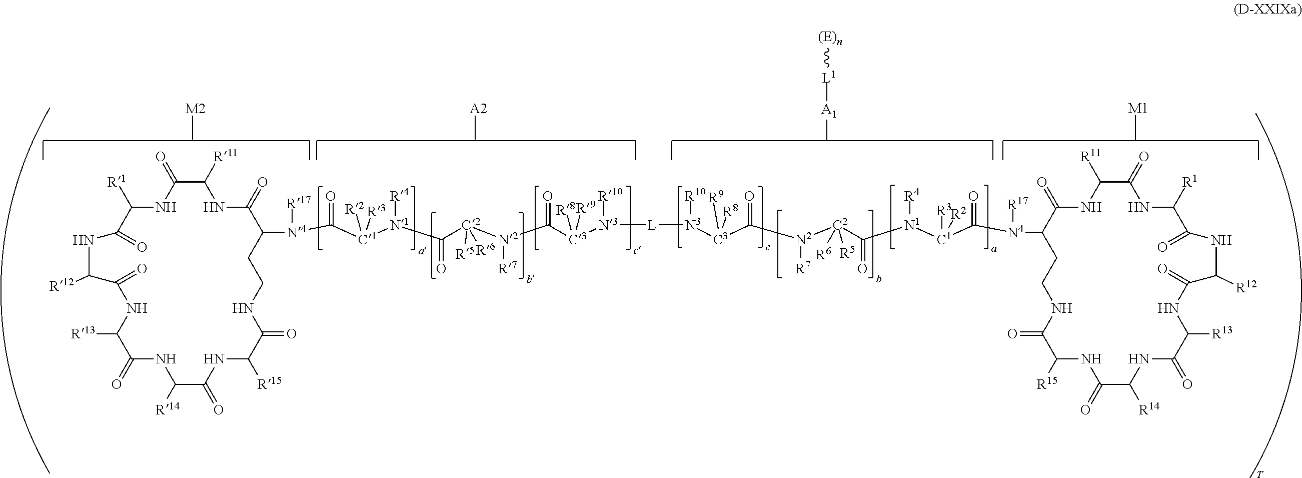

wherein each of L and L¹ is a remainder of L′; each of R¹, R¹², R′¹, and R′¹² is, independently, a lipophilic moiety, a polar moiety, or H; each of R¹¹, R¹³, R¹⁴, R′¹¹, R′¹³, and R′¹⁴ is, independently, optionally substituted C1-C5 alkamino, a polar moiety, a positively charged moiety, or H; each of R¹⁵ and R′¹⁵ is, independently, a lipophilic moiety or a polar moiety; each of R², R³, R⁴, R⁵, R⁶, R⁷, R⁸, R⁹, R¹⁰, R′², R′³, R′⁴, R′⁵, R′⁶, R′⁷, R′⁸, R′⁹, and R′¹⁰ is, independently, a lipophilic moiety, a positively charged moiety, a polar moiety, H, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; or two R groups on the same or adjacent atoms join to form an optionally substituted 3-8 membered ring (e.g., 5-8 membered ring); each of a′, b′, c′, b, and c is, independently, 0 or 1; each of N¹, N², N³, N⁴, N′¹, N′², N′³, and N′⁴ is a nitrogen atom; each of C¹, C², C³, C′¹, C′², and C′³ is a carbon atom, or a pharmaceutically acceptable salt thereof.

In some embodiments, the conjugate includes at least one optionally substituted 3-8 membered ring (e.g., 5-8 membered ring) formed by joining (i) R², R³, and C¹; (ii) R³, R⁴, N¹, and C¹; (iii) R⁵, R⁶, and C²; (iv) R⁶, R⁷, N², and C²; (v) R⁸, R⁹, and C³; (vi) R⁹, R¹⁰, N³, and C³; (vii) R′², R′³, and C′¹; (viii) R′³, R′⁴, N′¹, and C′¹; (ix) R′⁵, R′⁶, and C′²; (x) R′⁶, R′⁷, N′², and C′²; (xi) R′⁸, R′⁹, and C′³; or (xii) R′⁹, R′¹⁰, N′³, and C′³.

In some embodiments, the conjugate is described by formula (D-XXX):

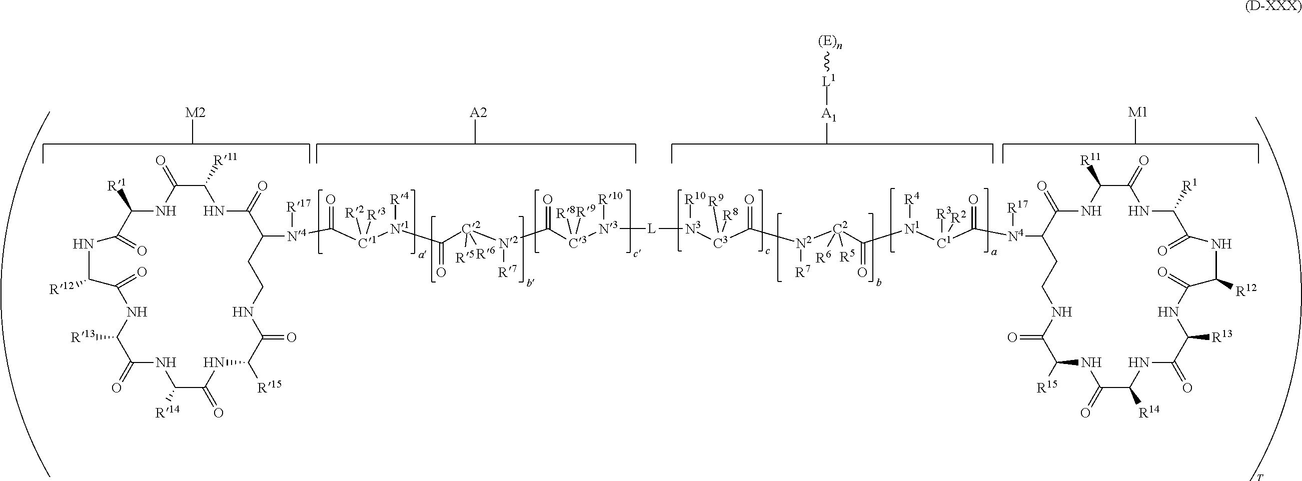

or a pharmaceutically acceptable salt thereof.

In some embodiments, each of R¹, R¹², R′¹, and R′¹² is, independently, a lipophilic moiety; each of R¹¹, R¹³, R¹⁴, R′¹¹, R′¹³, and R′¹⁴ is, independently, optionally substituted C1-C5 alkamino, a polar moiety, or a positively charged moiety; and/or each of R¹⁵ and R′¹⁵ is, independently, a polar moiety. In some embodiments, each of R¹ and R¹² is a lipophilic moiety. In some embodiments, each of R′¹ and R′¹² is a lipophilic moiety. In some embodiments, each lipophilic moiety is, independently, optionally substituted C1-C20 alkyl, optionally substituted C5-C15 aryl, optionally substituted C6-C35 alkaryl, or optionally C5-C10 substituted heteroaryl. In some embodiments, each lipophilic moiety is, independently, C1-C8 alkyl, methyl substituted C2-C4 alkyl, (C1-C10)alkylene(C6)aryl, phenyl substituted (C1-C10)alkylene(C6)aryl, or alkyl substituted C4-C9 heteroaryl. In some embodiments, each lipophilic moiety is, independently, benzyl, isobutyl, sec-butyl, isopropyl, n-propyl, methyl, biphenylmethyl, n-octyl, or methyl substituted indolyl.

In some embodiments, each of R¹¹, R¹³, R¹⁴, R′¹¹, R′¹³, and R′¹⁴ is independently optionally substituted C1-C5 alkamino (e.g., CH₂CH₂NH₂).

In some embodiments, each of R¹⁵ and R′¹⁵ is a polar moiety. In some embodiments, each polar moiety comprises a hydroxyl group, a carboxylic acid group, an ester group, or an amide group. In some embodiments, each polar moiety is hydroxyl substituted C1-C4 alkyl. In some embodiments, each polar moiety is CHCH₃OH.

In some embodiments of this aspect, the conjugate is described by formula (D-XXXI):

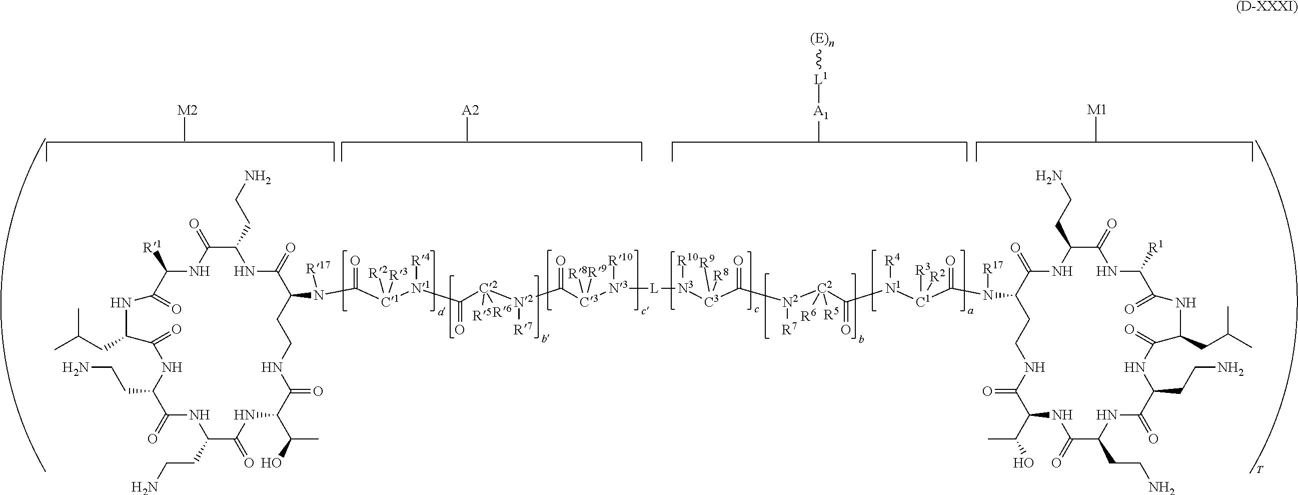

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In some embodiments, the conjugate is described by formula (D-XXXI-1):

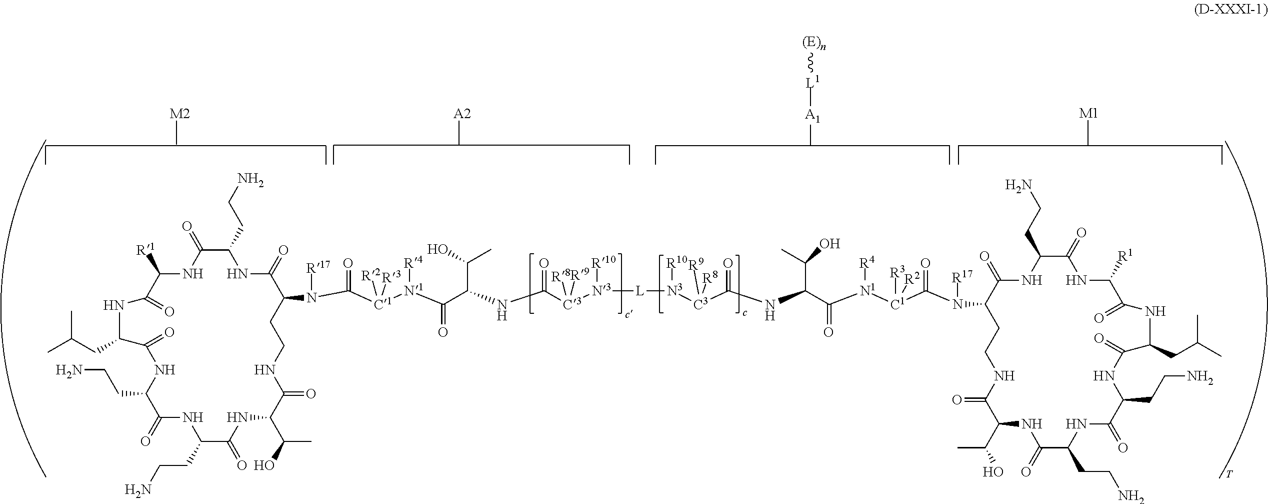

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In some embodiments, the conjugate is described by formula (D-XXXI-2):

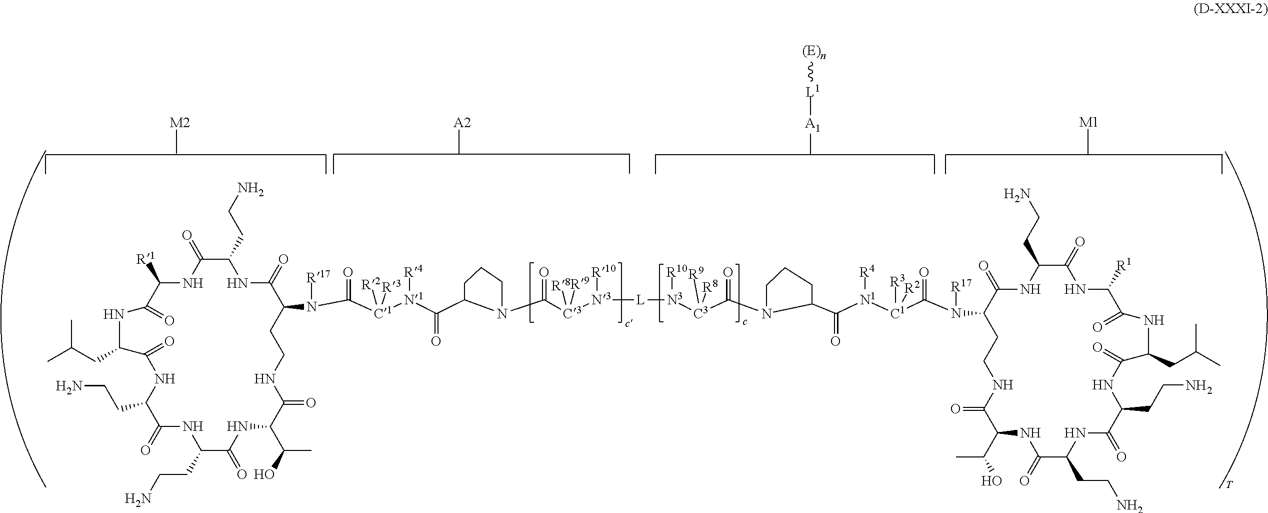

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In some embodiments, the conjugate is described by formula (D-XXXI-2a):

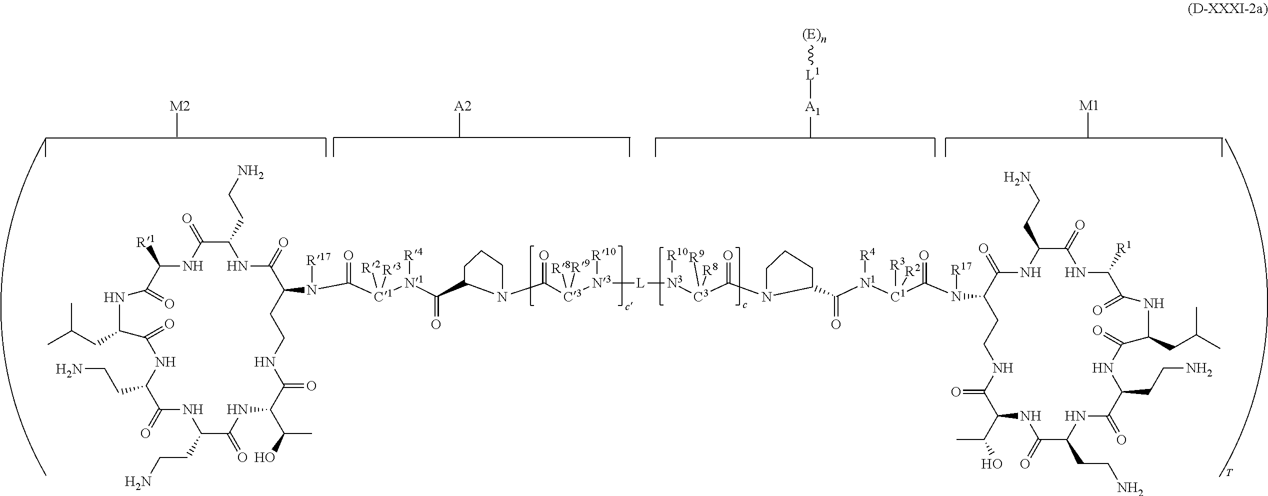

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In some embodiments, the conjugate is described by formula (D-XXXI-2b):

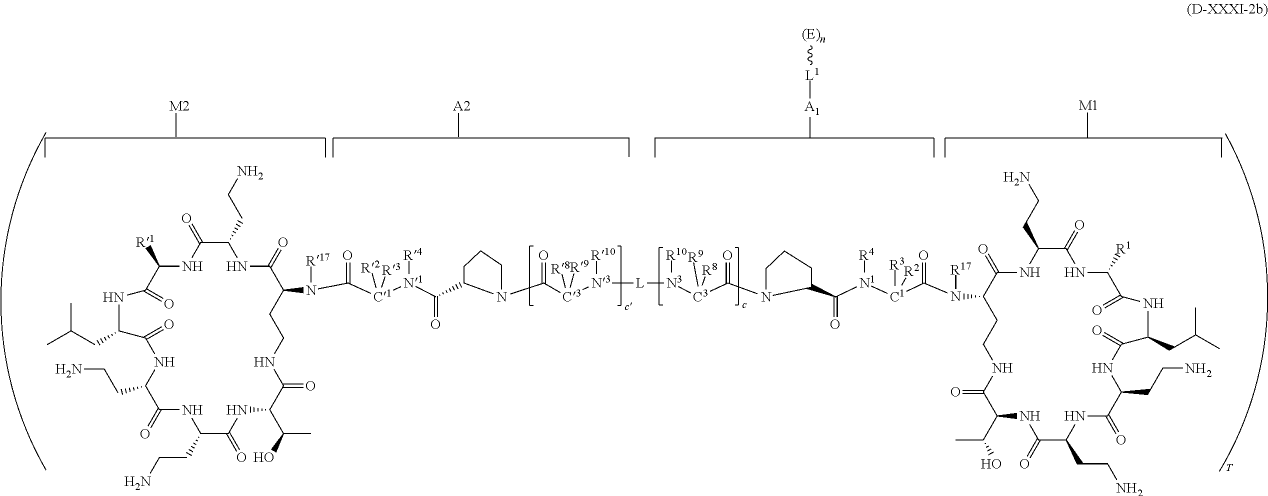

wherein each of R¹ and R′¹ is, independently, optionally substituted benzyl. CH₂CH(CH₃)₂, cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In some embodiments, the conjugate is described by formula (D-XXXI-3):

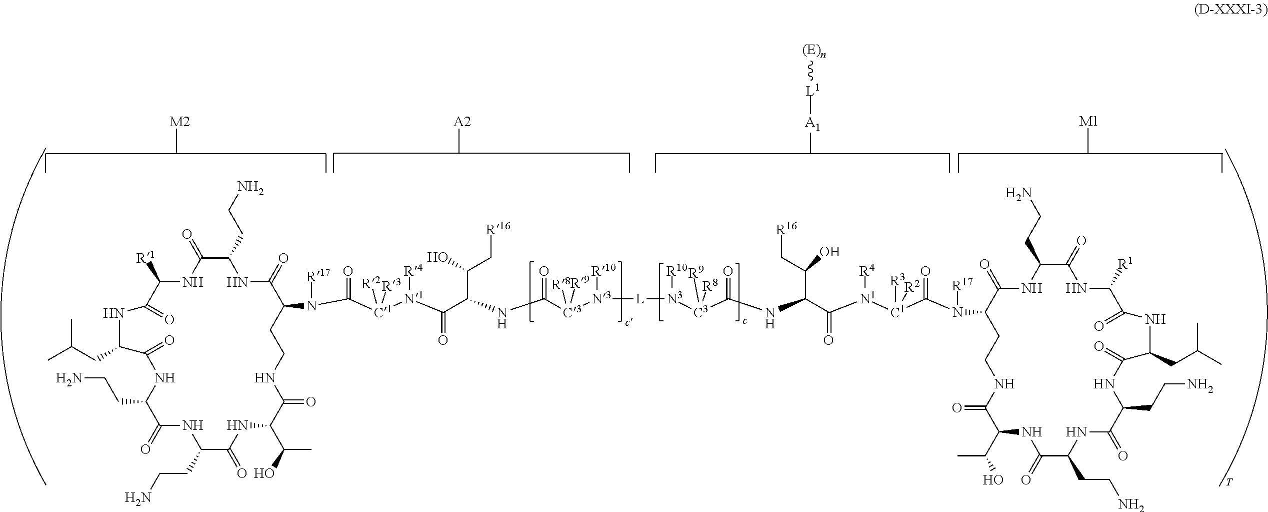

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R′¹⁶ and R¹⁶ is, independently, a lipophilic moiety, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments, each of R′¹⁶ and R¹⁶ is, independently, C1-C6 alkyl, benzyl, or phenethyl.

In some embodiments of this aspect the conjugate is described by formula (D-XXXII):

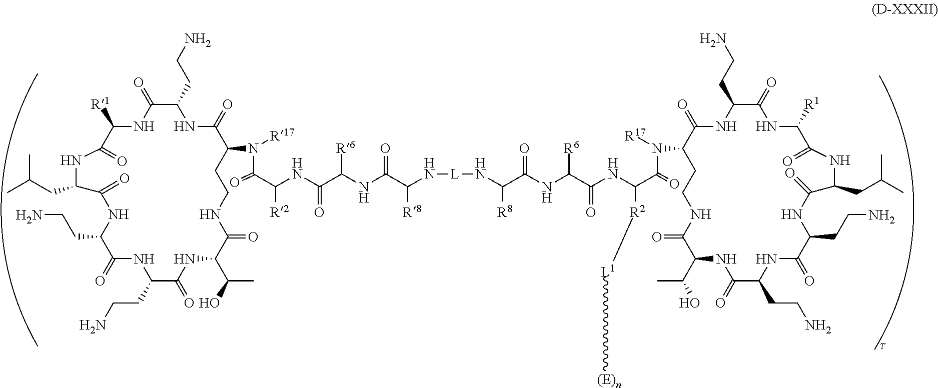

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R², R⁶, R⁸, R′², R′⁶, and R′⁸ is, independently, H. a lipophilic moiety, a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments of this aspect, the conjugate is described by formula (D-XXXIII):

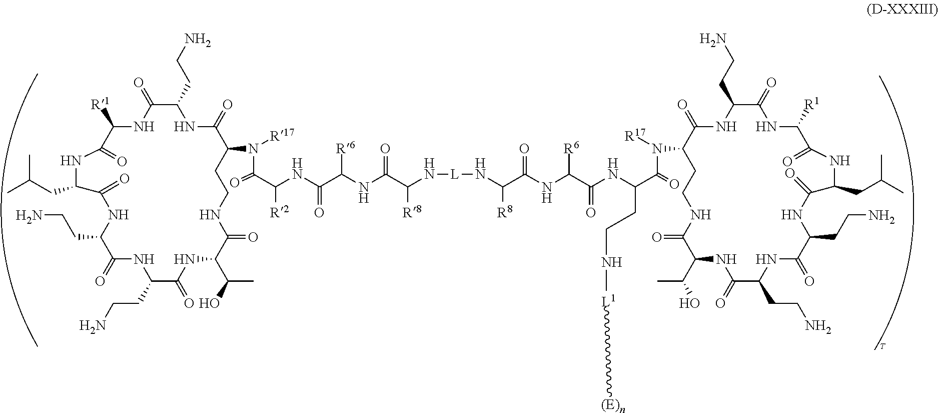

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R⁶, R⁸, R′², R′⁶, and R′⁸ is, independently, H, a lipophilic moiety, a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments of this aspect, the conjugate is described by formula (D-XXXIV):

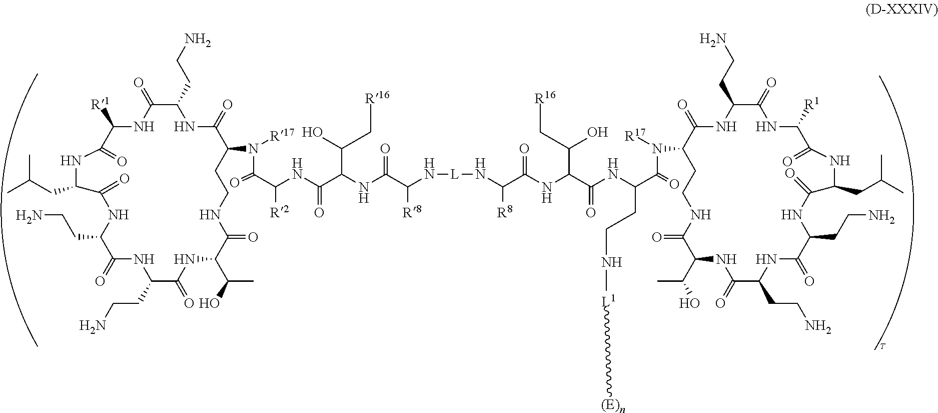

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R⁸, R′², and R′⁸ is, independently, H, a lipophilic moiety, a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; each of R′¹⁸ and R¹⁸ is, independently, H, a lipophilic moiety, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments, R′² is H, CH₂OH, CH₂CH₂NH, or CH₂CH₂CH₂CH(NH₂)COOH. In some embodiments, each of R⁸ and R′⁸ is CH₂CH₂NH₂. In some embodiments, each of R¹⁶ and R′¹⁶ is, independently, H, C1-C6 alkyl, benzyl, or phenethyl.

In some embodiments, the conjugate is described by formula (D-XXXIV-1):

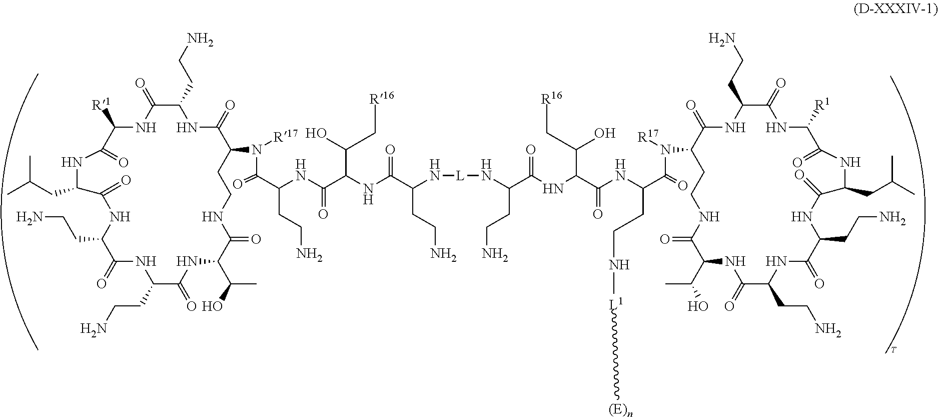

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R¹⁶ and R′¹⁶ is, independently, H, C1-C6 alkyl, benzyl, or phenethyl, or a pharmaceutically acceptable salt thereof.

In some embodiments, the conjugate is described by formula (D-XXXIV-2):

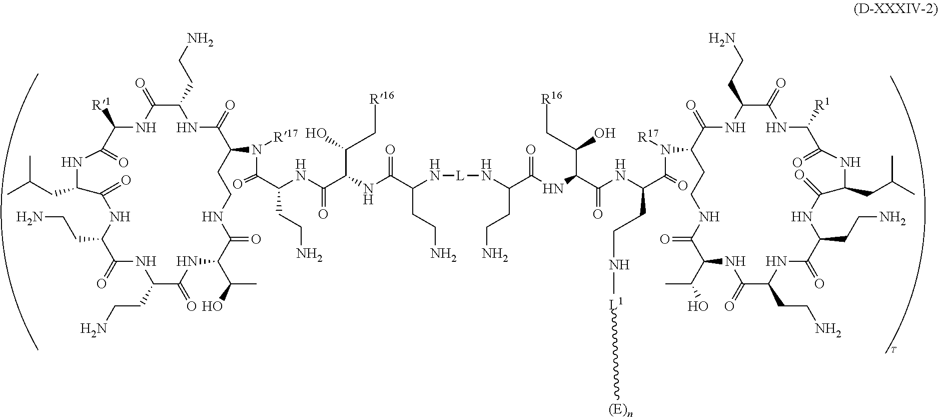

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R¹⁶ and R′¹⁶ is, independently, H, C1-C6 alkyl, benzyl, or phenethyl, or a pharmaceutically acceptable salt thereof.

In some embodiments, the conjugate is described by formula (D-XXXIV-3):

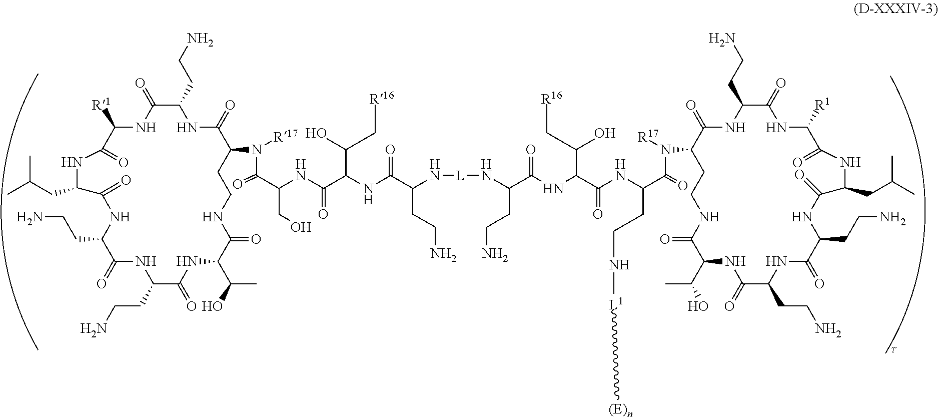

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R¹⁶ and R′¹⁶ is, independently, H, C1-C6 alkyl, benzyl, or phenethyl, or a pharmaceutically acceptable salt thereof.

In some embodiments, the conjugate is described by formula (D-XXXIV-5):

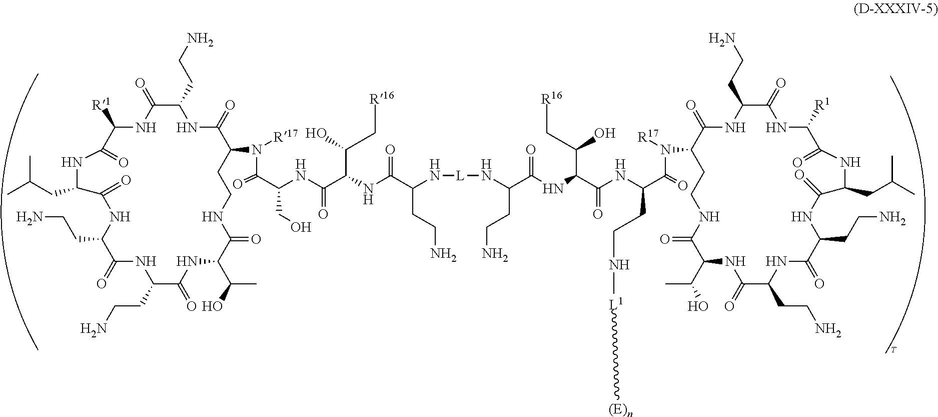

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R¹⁶ and R′¹⁶ is, independently, H, C1-C6 alkyl, benzyl, or phenethyl, or a pharmaceutically acceptable salt thereof.

In some embodiments, the conjugate is described by formula (D-XXXIV-6):

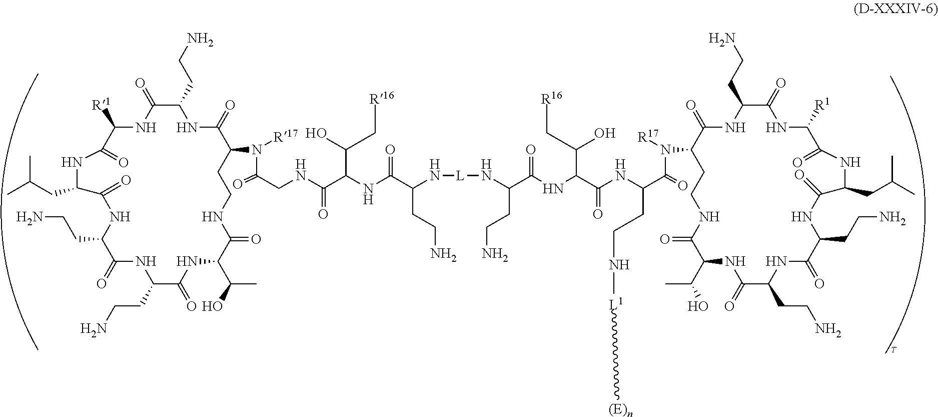

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R¹⁶ and R′¹⁶ is, independently, H, C1-C6 alkyl, benzyl, or phenethyl, or a pharmaceutically acceptable salt thereof.

In some embodiments, the conjugate is described by formula (D-XXXIV-7):

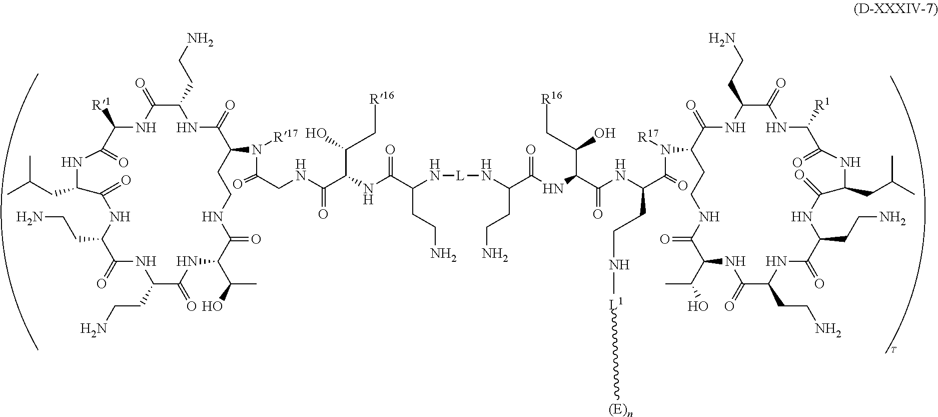

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R¹⁶ and R′¹⁶ is, independently, H, C1-C6 alkyl, benzyl, or phenethyl, or a pharmaceutically acceptable salt thereof.

In some embodiments, the conjugate is described by formula (D-XXXIV-8):

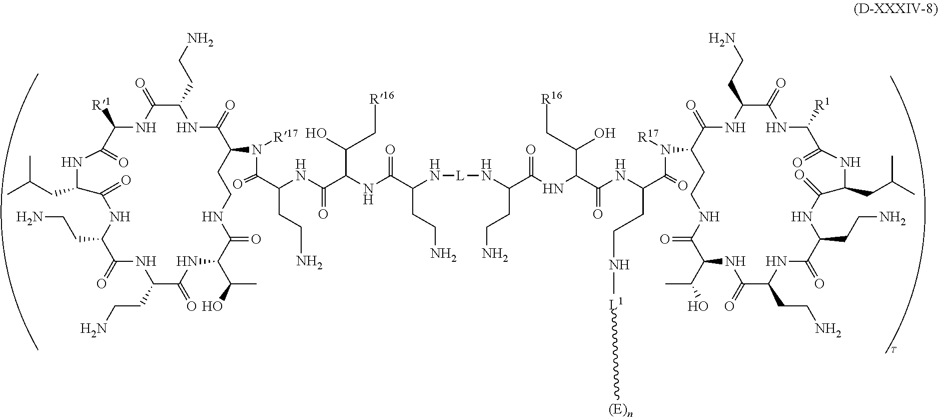

wherein each of R¹ and R′¹ is, independently, optionally substituted benzyl. CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R¹⁶ and R′¹⁶ is, independently. H, C1-C6 alkyl, benzyl, or phenethyl, or a pharmaceutically acceptable salt thereof.

In some embodiments, the conjugate is described b formula D-XXXIV-9):

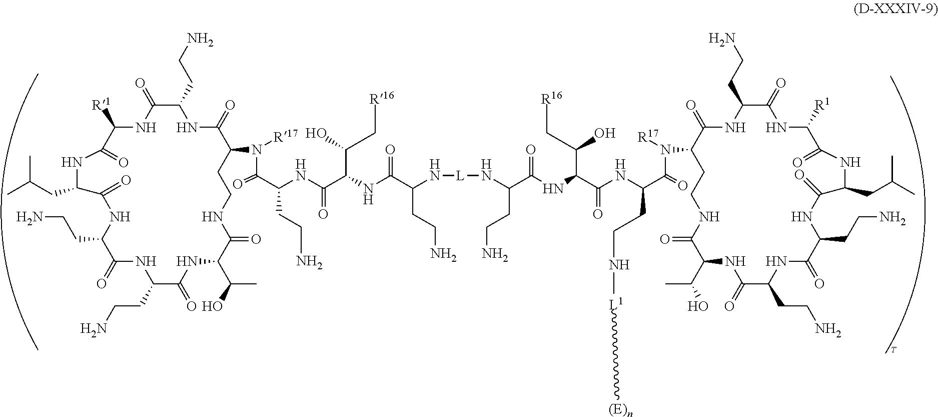

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R¹⁶ and R′¹⁶ is, independently. H, C1-C6 alkyl, benzyl, or phenethyl, or a pharmaceutically acceptable salt thereof.

In some embodiments, the conjugate is described by formula (D-XXXV):

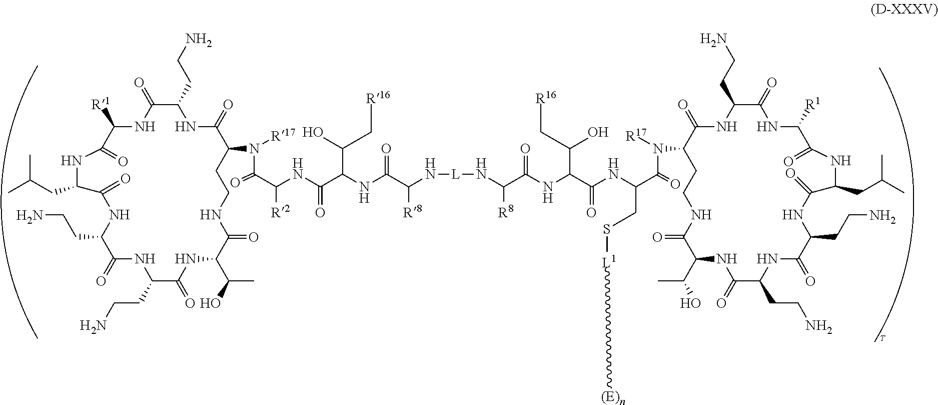

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; and each of R⁶, R⁸, R′², R′⁶, and R′⁸ is, independently, H, a lipophilic moiety, a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments, the conjugate is described by formula (D-XXXVI):

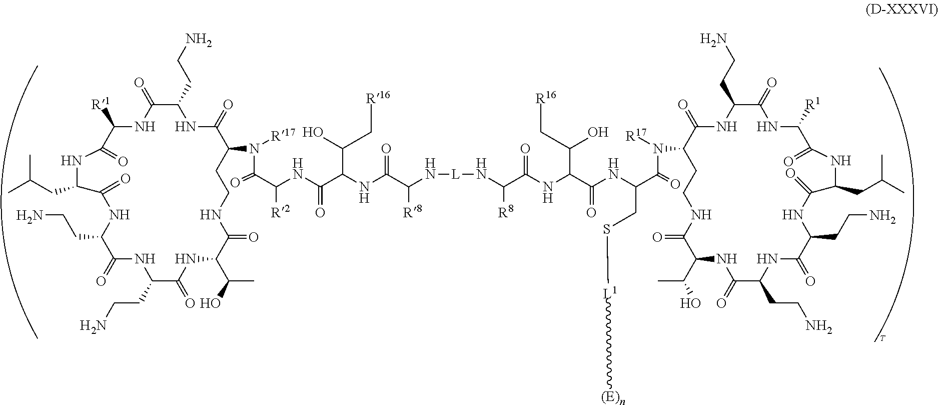

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R⁸, R′², and R′⁸ is, independently, H, a lipophilic moiety, a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; and each of R′¹⁶ and R¹⁶ is, independently, H, a lipophilic moiety, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; or a pharmaceutically acceptable salt thereof.

In some embodiments, R^{′2} is H, CH₂OH, CH₂CH₂NH, or CH₂CH₂CH₂CH(NH₂)COOH. In some embodiments, each of R⁸ and R′⁸ is CH₂CH₂NH₂. In some embodiments, each of R¹⁶ and R′¹⁶ is, independently, H, C1-C6 alkyl, benzyl, or phenethyl.

In some embodiments, the conjugate is described by formula (D-XXXVI-1):

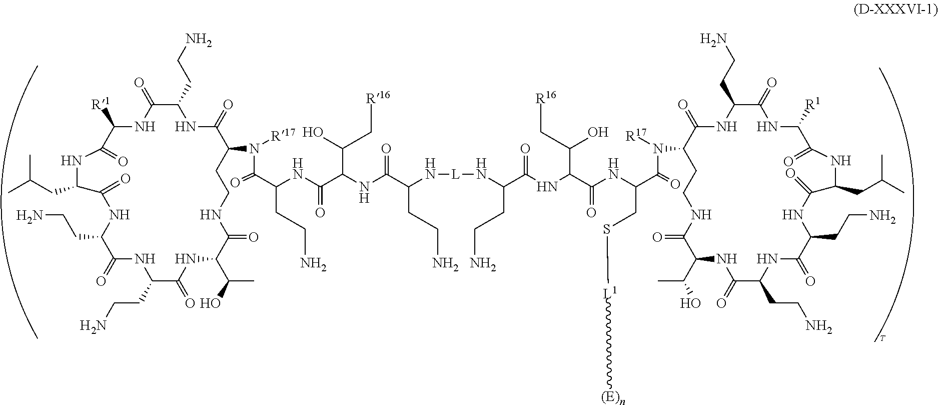

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; and each of R¹⁶ and R′¹⁶ is, independently, H, C1-C6 alkyl, benzyl, or phenethyl; or a pharmaceutically acceptable salt thereof.

In some embodiments, the conjugate is described by formula (D-XXXVI-2):

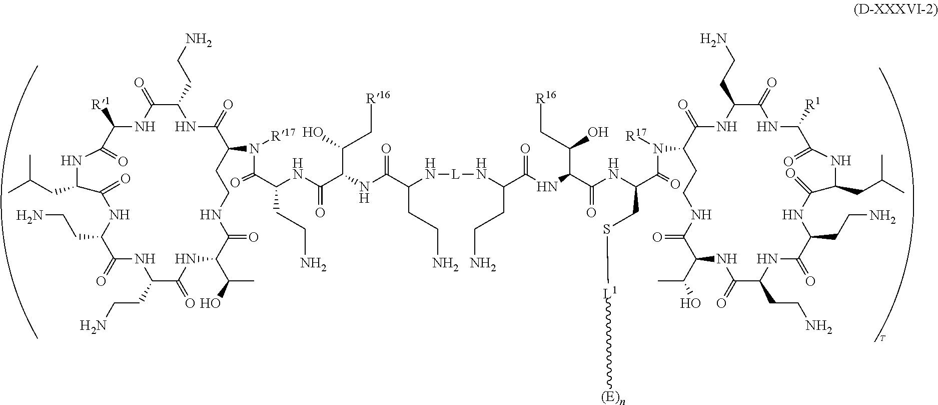

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; and each of R¹⁶ and R′¹⁶ is, independently, H, C1-C6 alkyl, benzyl, or phenethyl; or a pharmaceutically acceptable salt thereof.

In some embodiments, the conjugate is described by formula (D-XXXVI-2a):

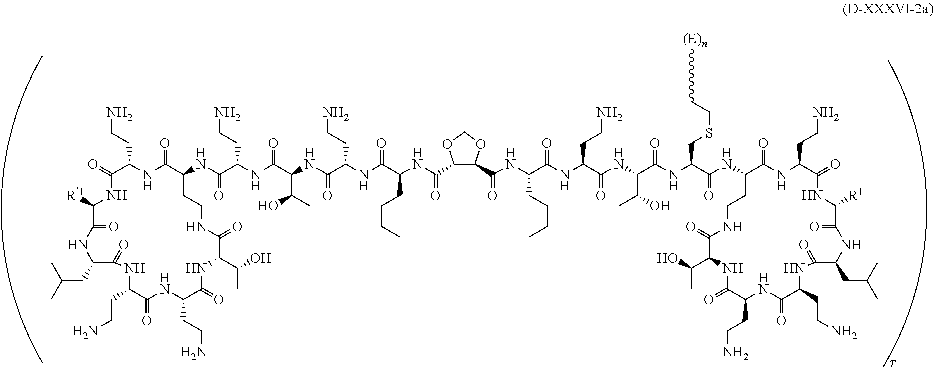

wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, each of R¹⁷ and R′¹⁷ is H. In some embodiments, each of R¹⁷ and R′¹⁷ is methyl.

In some embodiments, each of R¹ and R′¹ is, independently, benzyl or CH₂CH(CH₃)₂. In some embodiments, R¹ and R′¹ are each benzyl. In some embodiments, R¹ and R′¹ are each CH₂CH(CH₃)₂.

In some embodiments, L′, L, or L¹ includes one or more optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, optionally substituted C2-C15 heteroarylene, O, S, NRⁱ, P, carbonyl, thiocarbonyl, sulfonyl, phosphate, phosphoryl, or imino, wherein Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C3-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl.

In some embodiments, the backbone of L′, L, or L¹ consists of one or more optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, optionally substituted C2-C15 heteroarylene, O, S, NRⁱ, P, carbonyl, thiocarbonyl, sulfonyl, phosphate, phosphoryl, or imino, wherein Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C3-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl.

In some embodiments, L′, L, or L′¹ is oxo substituted. In some embodiments, the backbone of L′, L, or L¹ includes no more than 250 atoms. In some embodiments, L′, L, or L¹ is capable of forming an amide, a carbamate, a sulfonyl, or a urea linkage. In some embodiments, L or L¹ is a bond.

In some embodiments, each L is described by formula (D-L-II):

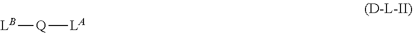

wherein L^{A} is described by formula G^{A1}-(Z^{A1})_{g1}-(Y^{A1})ₕ₁-(Z^{A2})ᵢ₁-(Y^{A2})ⱼ₁-(Z^{A2})ₖ₁-(Y^{A3})ₗ₁-(Z^{A4})ₘ₁-(Y^{A4})ₙ₁-(Z^{A5})_{O1}-G^{A2}; L^{B} is described by formula G^{B1}-(Z^{B1})_{g2}-(Y^{B1})ₕ₂-(Z^{B2})ᵢ₂-(Y^{B2})ⱼ₂-(Z^{B3})ₖ₂-(Y^{B3})ₗ₂-(Z^{B4})ₘ₂-(Y^{B4})ₙ₂-(Z^{B5})ₒ₂-G^{B2}; G^{A1} is a bond attached to Q; G^{A2} is a bond attached to A1; G^{B1} is a bond attached to Q; G^{B2} is a bond attached to A2 or M2 if A2 is absent; each of Z^{A1}, Z^{A2}, Z^{A3}, Z^{A4}, Z^{A5}, Z^{B1}, Z^{B2}, Z^{B3}, Z^{B4}, and Z^{B5} is independently, optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene; each of Y^{A1}, Y^{A2}, Y^{A3}, Y^{A4}, Y^{B1}, Y^{B2}, Y^{B3}, and Y^{B4} is, independently, O, S, NRⁱ, P, carbonyl, thiocarbonyl, sulfonyl, phosphate, phosphoryl, or imino; Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C3-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl; each of g1, h1, i1, j1, k1, l1, m1, n1, o1, g2, h2, i2, j2, k2, l2, m2, n2, and o2 is independently, 0 or 1; Q is a nitrogen atom, optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene.

In some embodiments, depending on the structure and chemical formula of L^{C}. L^{C} may have two points of attachment to the Fc domain (e.g., two G^{C2}).

In some embodiments, L is

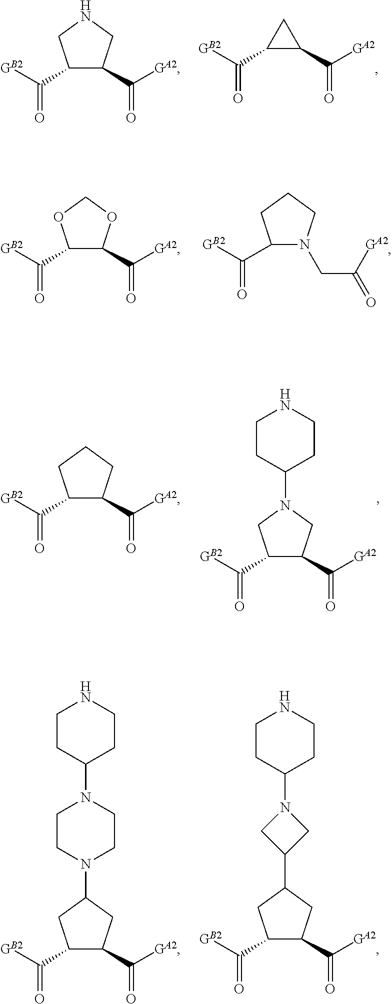

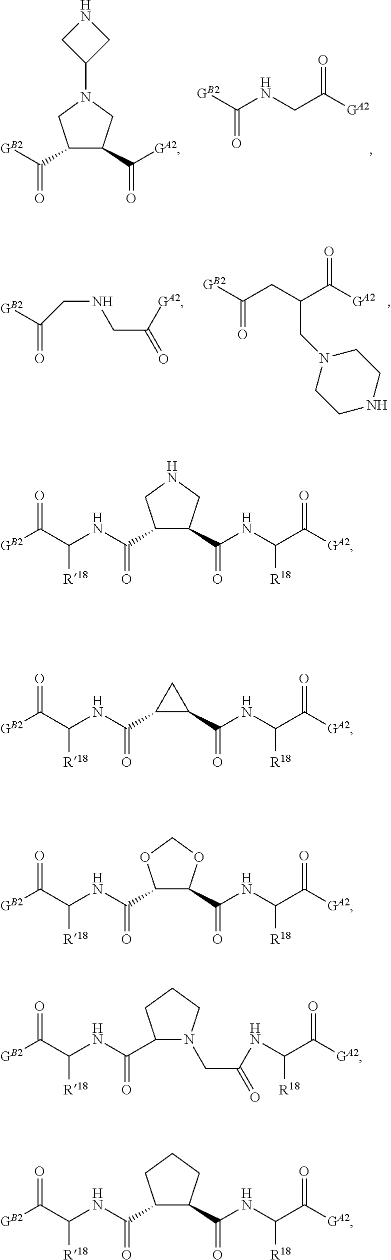

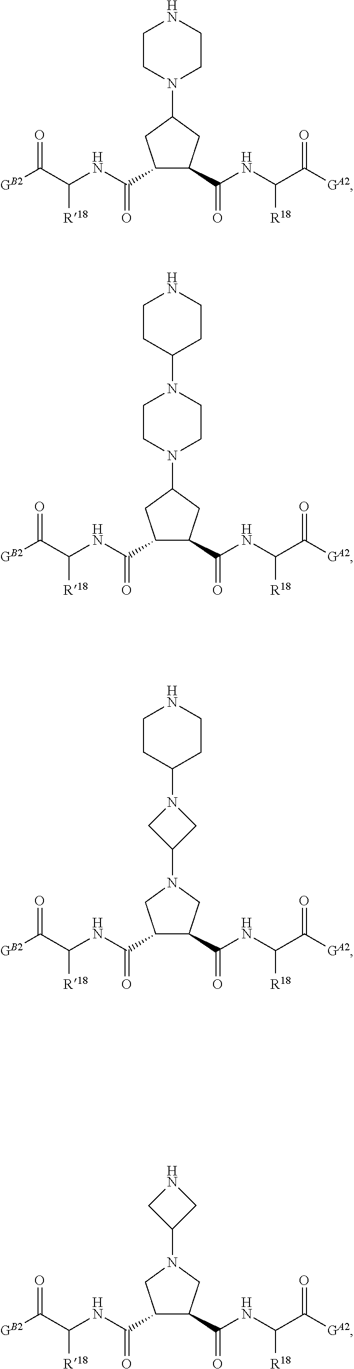

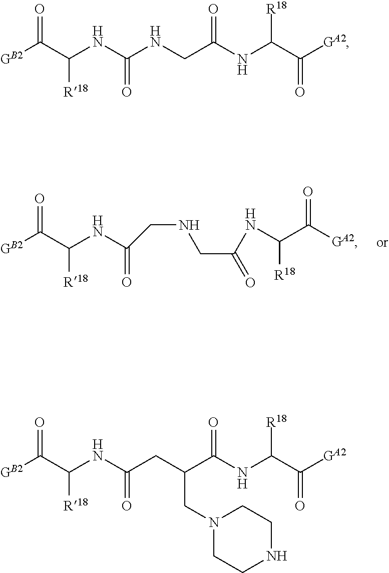

wherein each of R′¹⁸ and R¹⁸ is, independently, H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C3-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl.

In some embodiments, each of R′¹⁸ and R¹⁸ is, independently, butyl, cyclohexyl, isopropyl, or isobutyl.

In some embodiments, each L¹ is described by formula (D-L-Ill):

J¹-(S¹)_{g}-(T¹)ₕ-(S²)ᵢ-(T²)ⱼ-(S³)ₖ-(T³)ₗ-(S⁴)ₘ-(T⁴)ₙ-(S⁵)ₒ-J² (D-L-III)

wherein J¹ is a bond attached to A1; J² is a bond attached to E; each of S¹, S², S³, S⁴, and S⁵ is, independently, optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene; each of T¹, T², T³, T4 is, independently, O, S. NRⁱ, P, carbonyl, thiocarbonyl, sulfonyl, phosphate, phosphoryl, or imino; Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C3-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl; and each of g, h, i, j, k, l, m, n, and o is, independently, 0 or 1, or a pharmaceutically acceptable salt thereof.

In some embodiments, L¹ is

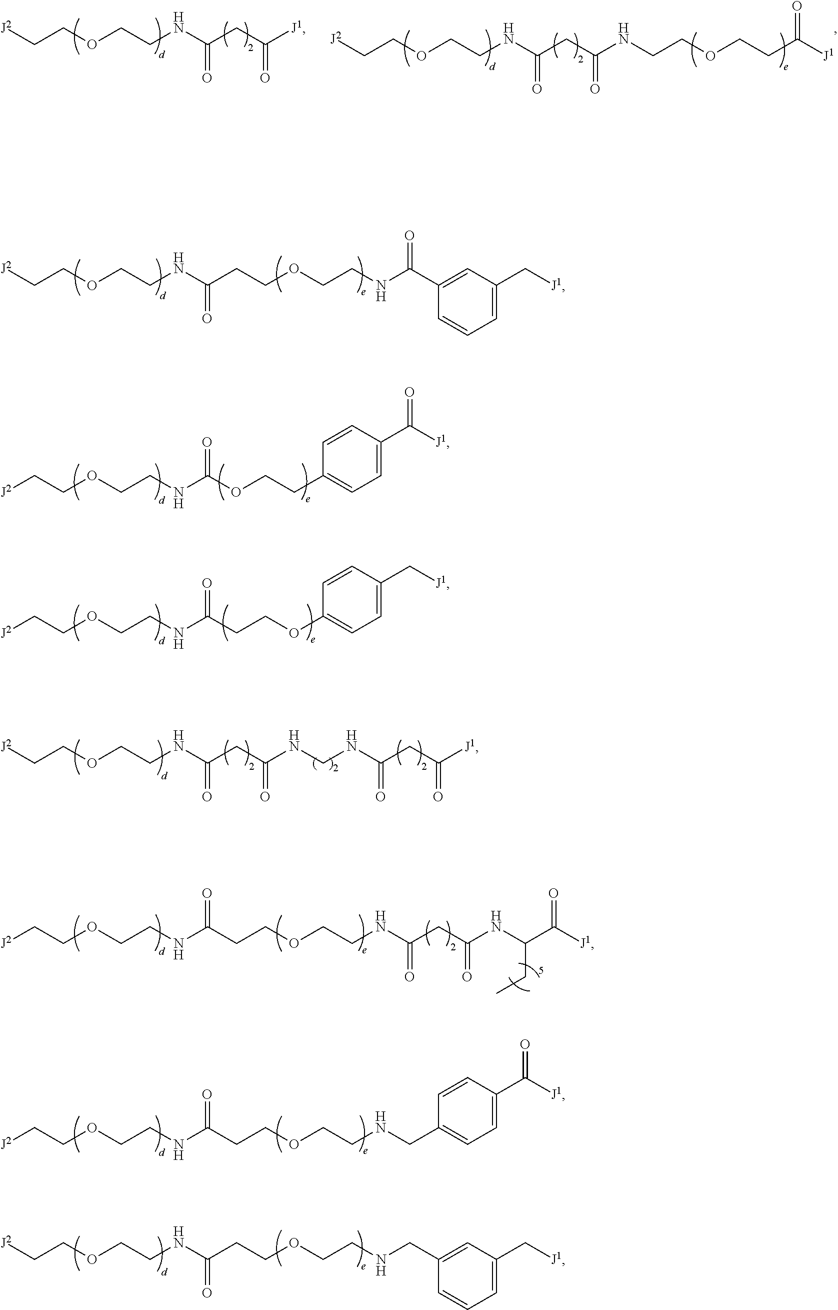

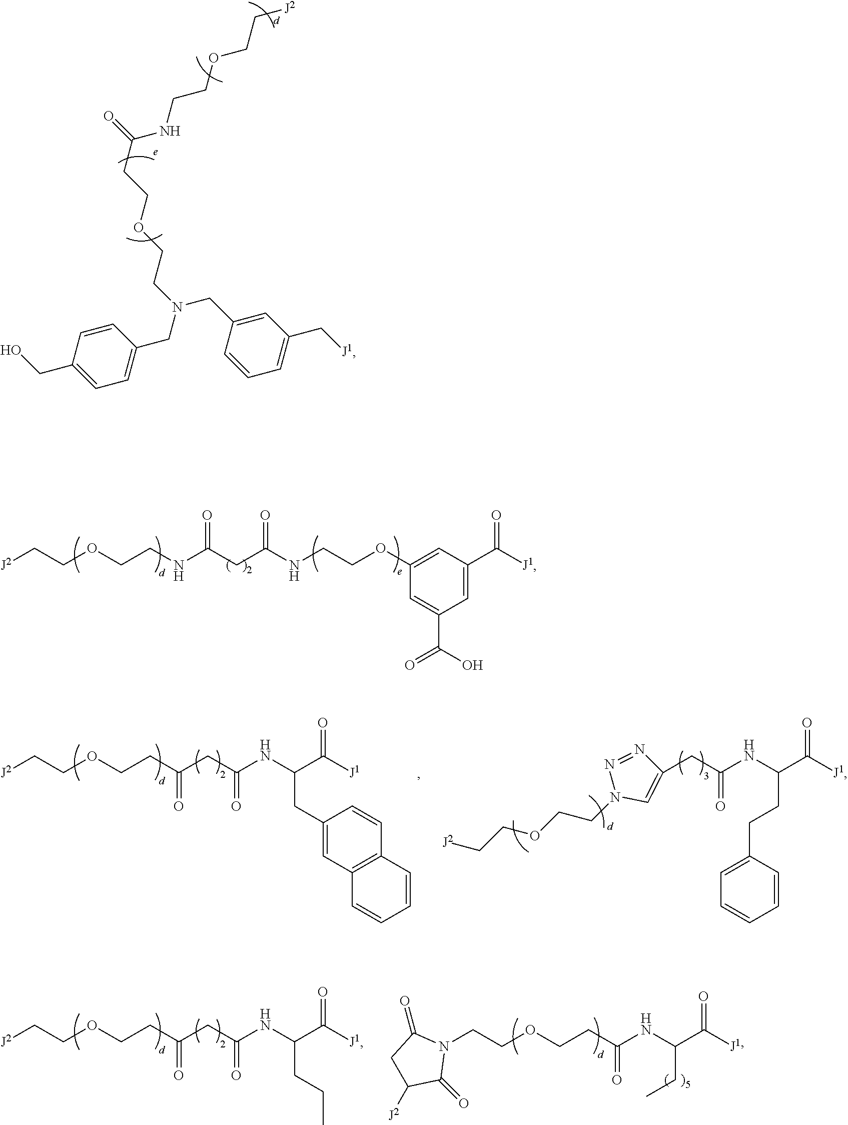

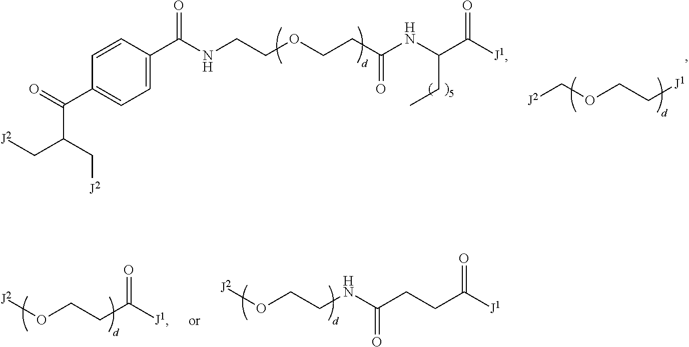

wherein each of d and e is, independently, an integer from 1 to 26, or a pharmaceutically acceptable salt thereof.

In some embodiments. E has the sequence of any one of SEQ ID NOs: 1-14, 1-29, or 1-31. In some embodiments where n is 2. E dimerizes to form an Fc domain.

In another aspect, the invention features a conjugate of formula (D-XV):

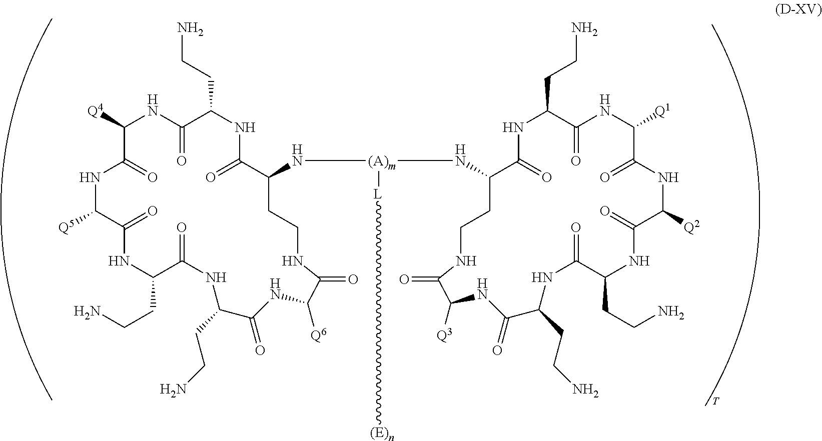

wherein: each A is an independently selected amino acid; each E is an Fc domain monomer, each L is a linker that, when each m is 2, 3, 4, or 5, is bound to any of A and covalently attached to E; each m is 0, 1, 2, 3, 4, or 5; n is 1 or 2; T is an integer from 1 to 20, e.g., from 1 to 15; and Q¹, Q², Q³, Q⁴, Q⁵ and Q⁶ are each independently selected from the side chain of an amino acid; or a pharmaceutically acceptable salt thereof.

In another aspect, the invention features a conjugate of formula (D-XVI):

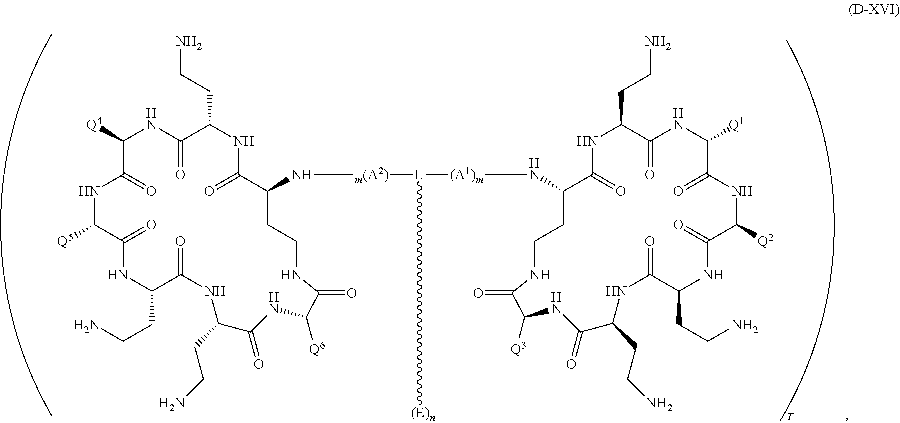

wherein: each A¹ and A² is an independently selected amino acid; each L is a linker that, when each m is 1, 2, 3, 4, or 5, is bound to a nitrogen atom in any A¹ and a nitrogen atom in any A² and covalently linked to E; each E is an Fc domain monomer; each m is independently 0, 1, 2, 3, 4, or 5; n is 1 or 2; T is an integer from 1 to 20, e.g., from 1 to 15; and Q¹, Q², Q³, Q⁴, Q⁵ and Q⁶ are each independently selected from the side chain of an amino acid; or a pharmaceutically acceptable salt thereof.

In some embodiments of this aspect, each A¹ and A² is independently selected from glycine, arginine, asparagine, glutamine, 3-(2H-tetrazol-5-yl)alanine, 3-aminoalanine, piperazine-2-carboxylic acid, 2,4-diaminobutyric acid, 3-hydroxyproline, threonine, 2-amino-4-phenylbutyric acid, 3-(2-naphthyl)alanine, 2-piperazinecarboxylic acid, 2-aminooctanoic acid, serine, 2-aminohexanoic acid, 4-amino-4-piperidinyl carboxylic acid, methionine, methionine sulfoxide, methionine sulfone, S-methylcysteine, S-ethylcysteine, S-propylhomocysteine, cyclopropylalanine, 3-fluoroalanine, 2-amino-5-methylhexanoic acid, 2-amino-5-methylhex-4-enoic acid, alpha-t-butylglycine, and alpha-neopentylglycine; and each m is independently selected from 1, 2, 3, 4, or 5; or a pharmaceutically acceptable salt thereof.

In some embodiments of this aspect, each m is independently 2, 3, or 4, or a pharmaceutically acceptable salt thereof.

In some embodiments of this aspect, each A¹ and A² is independently selected from glycine, arginine, asparagine, glutamine, 3-(2H-tetrazol-5-yl)alanine, 3-aminoalanine, piperazine-2-carboxylic acid, 2,4-diaminobutyric acid, 3-hydroxyproline, threonine, 2-amino-4-phenylbutyric acid, 3-(2-naphthyl)alanine, 2-piperazinecarboxylic acid, 2-aminooctanoic acid, serine, 2-aminohexanoic acid, 4-amino-4-piperidinyl carboxylic acid, methionine, methionine sulfoxide, methionine sulfone, S-methylcysteine, S-ethylcysteine, S-propylhomocysteine, cyclopropylalanine, 3-fluoroalanine, 2-amino-5-methylhexanoic acid, 2-amino-5-methylhex-4-enoic acid, alpha-t-butylglycine, and alpha-neopentylglycine; each m is independently 1, 2, 3 or 4; the combination of Q¹, Q², Q³, Q⁴, Q⁵ and Q⁶ is selected from one of

| Q¹ | Q² | Q³ | Q⁴ | Q⁵ | Q⁶ |
|---|---|---|---|---|---|
| 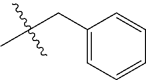 [SMILES: *Cc1ccccc1] | 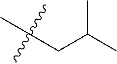 [SMILES: *CC(C)C] | 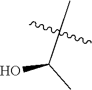 [SMILES: *[C@@H](C)O] | 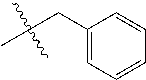 [SMILES: *Cc1ccccc1] | 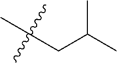 [SMILES: *CC(C)C] | 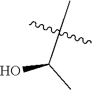 [SMILES: *[C@@H](C)O] |
| 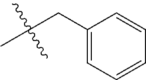 [SMILES: *Cc1ccccc1] | 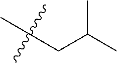 [SMILES: *CC(C)C] | 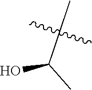 [SMILES: *[C@@H](C)O] | 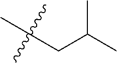 [SMILES: *CC(C)C] | 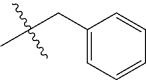 [SMILES: *Cc1ccccc1] | 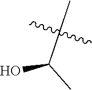 [SMILES: *[C@@H](C)O] |
| 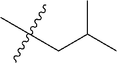 [SMILES: *CC(C)C] | 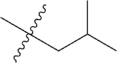 [SMILES: *CC(C)C] | 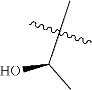 [SMILES: *[C@@H](C)O] | 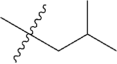 [SMILES: *CC(C)C] | 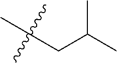 [SMILES: *CC(C)C] | 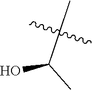 [SMILES: *[C@@H](C)O] |

or a pharmaceutically acceptable salt thereof.

In another aspect, the invention features a conjugate of formula (D-XVII):

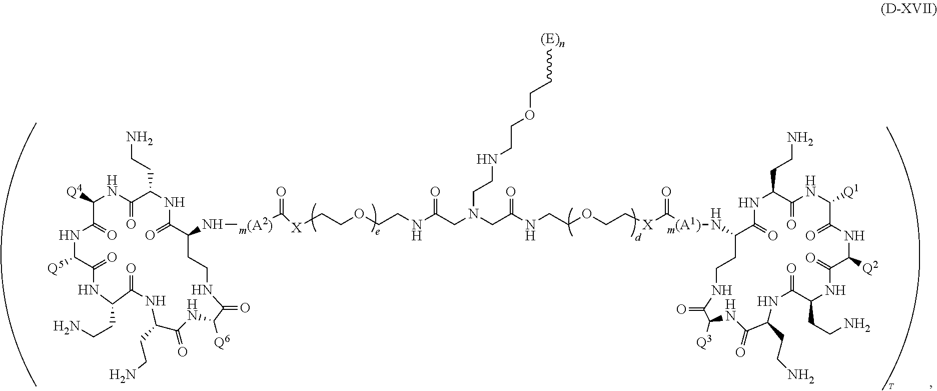

wherein: each A¹ and A² is an independently selected amino acid; each X is absent or is —CH₂CH₂C(O)NH—; each E is an Fc domain monomer; each m is independently 0, 1, 2, 3, 4, or 5; n is 1 or 2; Q¹, Q², Q³, Q⁴, Q⁵ and Q⁶ are each independently selected from the side chain of an amino acid; each d is an integer from 0 to 10; each e is an integer from 0 to 10; and T is an integer from 1 to 20, e.g., from 1 to 15, or a pharmaceutically acceptable salt thereof.

In some embodiments of this aspect, each A¹ and A² is independently selected from glycine, arginine, asparagine, glutamine, 3-(2H-tetrazol-5-yl)alanine, 3-aminoalanine, piperazine-2-carboxylic acid, 2,4-diaminobutyric acid, 3-hydroxyproline, threonine, 2-amino-4-phenylbutyric acid, 3-(2-naphthyl)alanine, 2-piperazinecarboxylic acid, 2-aminooctanoic acid, serine, 2-aminohexanoic acid, 4-amino-4-piperidinyl carboxylic acid, methionine, methionine sulfoxide, methionine sulfone, S-methylcysteine, S-ethylcysteine, S-propylhomocysteine, cyclopropylalanine, 3-fluoroalanine, 2-amino-5-methylhexanoic acid, 2-amino-5-methylhex-4-enoic acid, alpha-t-butylglycine, and alpha-neopentylglycine; each m is 2 or 3; each d is an integer from 1 to 10; each e is an integer from 1 to 10; or a pharmaceutically acceptable salt thereof.

In some embodiments of this aspect, each A¹ and A² is independently selected from 2,4-diaminobutyric acid and threonine; each m is 2 or 3; each d is an integer from 1 to 10; and each e is an integer from 1 to 10; or a pharmaceutically acceptable salt thereof.

In some embodiments of this aspect, each m is 2, or a pharmaceutically acceptable salt thereof. In some embodiments, each m is 3, or a pharmaceutically acceptable salt thereof.

In some embodiments of this aspect, each d is 10; each e is 10; and each X is absent; or a pharmaceutically acceptable salt thereof.

In some embodiments of this aspect, each m is 2; each d is 1; each e is 1; and each X is —CH₂CH₂C(O)NH—; or a pharmaceutically acceptable salt thereof.

In another aspect, the invention features a conjugate of formula (D-XVIII):

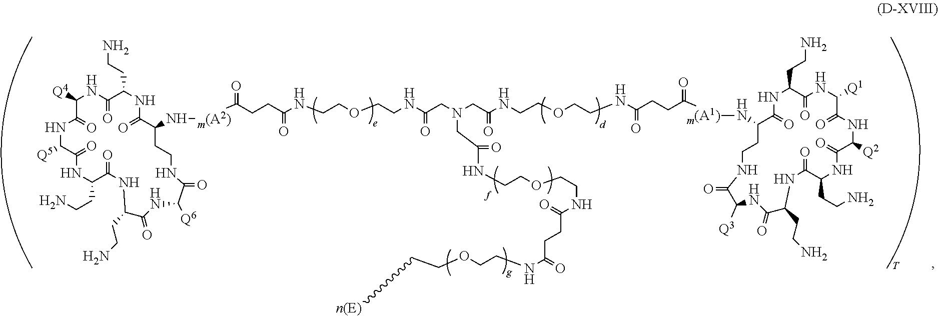

wherein: each A¹ and A² is an independently selected amino acid; each E is an Fc domain monomer; each m is independently 0, 1, 2, 3, 4, or 5; n is 1 or 2; Q¹, Q², Q³, Q⁴, Q⁵ and Q⁶ are each independently selected from the side chain of an amino acid; each d is an integer from 0 to 10; each e is an integer from 0 to 10; each f is an integer from 0 to 10; each g is an integer from 0 to 10; and T is an integer from 1 to 20, e.g., from 1 to 15, or a pharmaceutically acceptable salt thereof.

In some embodiments of this aspect, each A¹ and A² is independently selected from glycine, arginine, asparagine, glutamine, 3-(2H-tetrazol-5-yl)alanine, 3-aminoalanine, piperazine-2-carboxylic acid, 2,4-diaminobutyric acid, 3-hydroxyproline, threonine, 2-amino-4-phenylbutyric acid, 3-(2-naphthyl)alanine, 2-piperazinecarboxylic acid, 2-aminooctanoic acid, serine, 2-aminohexanoic acid, 4-amino-4-piperidinyl carboxylic acid, methionine, methionine sulfoxide, methionine sulfone. S-methylcysteine, S-ethylcysteine, S-propylhomocysteine, cyclopropylalanine, 3-fluoroalanine, 2-amino-5-methylhexanoic acid, 2-amino-5-methylhex-4-enoic acid, alpha-t-butylglycine, and alpha-neopentylglycine; each m is 2; each d is 1; each e is 1; each f is 3; and each g is 1; or a pharmaceutically acceptable salt thereof.

In some embodiments of this aspect, each A¹ and A² is independently selected from 2,4-diaminobutyric acid and threonine; each m is 2; each d is 1; each e is 1; each f is 3; and each g is 1; or a pharmaceutically acceptable salt thereof.

In another aspect, the invention features a conjugate of formula (D-XIX):

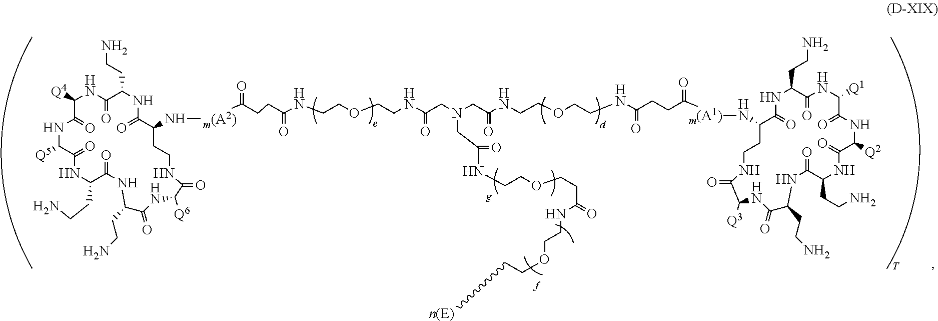

wherein: each A¹ and A² is an independently selected amino acid; each E is an Fc domain monomer; each m is independently 0, 1, 2, 3, 4, or 5; n is 1 or 2; Q¹, Q², Q³, Q⁴, Q⁵ and Q⁶ are each independently selected from the side chain of an amino acid; each d is an integer from 0 to 10; each e is an integer from 0 to 10; each f is an integer from 0 to 10; each g is an integer from 0 to 25; and T is an integer from 1 to 20, e.g., from 1 to 15, or a pharmaceutically acceptable salt thereof.

In some embodiments, each A¹ and A² is independently selected from glycine, arginine, asparagine, glutamine, 3-(2H-tetrazol-5-yl)alanine, 3-aminoalanine, piperazine-2-carboxylic acid, 2,4-diaminobutyric acid, 3-hydroxyproline, threonine, 2-amino-4-phenylbutyric acid, 3-(2-naphthyl)alanine, 2-piperazinecarboxylic acid, 2-aminooctanoic acid, serine, 2-aminohexanoic acid, 4-amino-4-piperidinyl carboxylic acid, methionine, methionine sulfoxide, methionine sulfone, S-methylcysteine, S-ethylcysteine, S-propylhomocysteine, cyclopropylalanine, 3-fluoroalanine, 2-amino-5-methylhexanoic acid, 2-amino-5-methylhex-4-enoic acid, alpha-t-butylglycine, and alpha-neopentylglycine; each m is 2; each d is 1; each e is 1; each f is 1; and each g is 8 to 25; or a pharmaceutically acceptable salt thereof.

In some embodiments of this aspect, each A¹ and A² is independently selected from 2,4-diaminobutyric acid and threonine; each m is 2; each d is 1; each e is 1; each f is 1; and each g is 8 to 25; or a pharmaceutically acceptable salt thereof.

In another aspect, the invention features a conjugate of formula (D-XX):

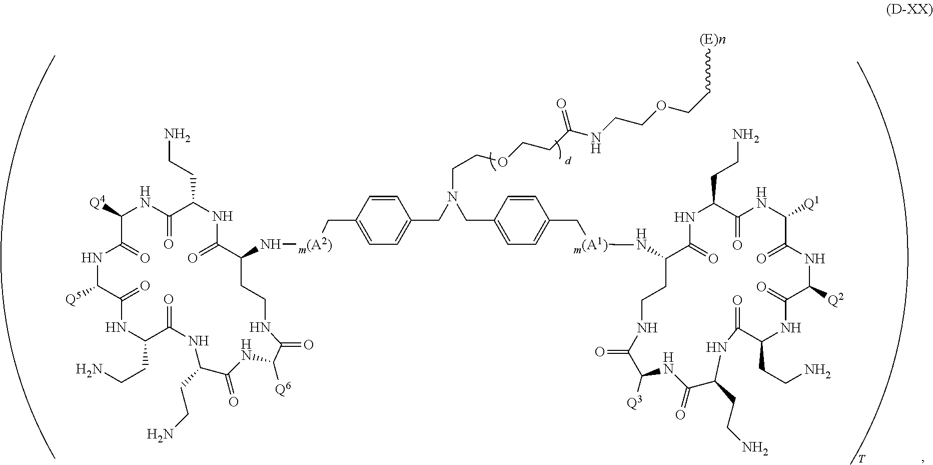

wherein: each A¹ and A² is an independently selected amino acid; each E is an Fc domain monomer; each m is independently 0, 1, 2, 3, 4, or 5; n is independently 1 or 2; Q¹, Q², Q³, Q⁴, Q⁵ and Q⁶ are each independently selected from the side chain of an amino acid; each d is an integer from 0 to 15; and T is an integer from 1 to 20, e.g., from 1 to 15, or a pharmaceutically acceptable salt thereof.

In some embodiments of this aspect, each A¹ and A² is independently selected from glycine, arginine, asparagine, glutamine, 3-(2H-tetrazol-5-yl)alanine, 3-aminoalanine, piperazine-2-carboxylic acid, 2,4-diaminobutyic acid, 3-hydroxyproline, threonine, 2-amino-4-phenylbutyric acid, 3-(2-naphthyl)alanine, 2-piperazinecarboxylic acid, 2-aminooctanoic acid, serine, 2-aminohexanoic acid, 4-amino-4-piperidinyl carboxylic acid, methionine, methionine sulfoxide, methionine sulfone, S-methylcysteine, S-ethylcysteine, S-propylhomocysteine, cyclopropylalanine, 3-fluoroalanine, 2-amino-5-methylhexanoic acid, 2-amino-5-methylhex-4-enoic acid, alpha-t-butylglycine, and alpha-neopentylglycine; each m is 2; and each d is 10; or a pharmaceutically acceptable salt thereof.

In some embodiments of this aspect, each A¹ and A² is independently selected from 2,4-diaminobutyric acid and piperazine-2-carboxylic acid; each m is 2; and each d is 10; or a pharmaceutically acceptable salt thereof.

In another aspect, the invention features a conjugate of formula (D-XXI):

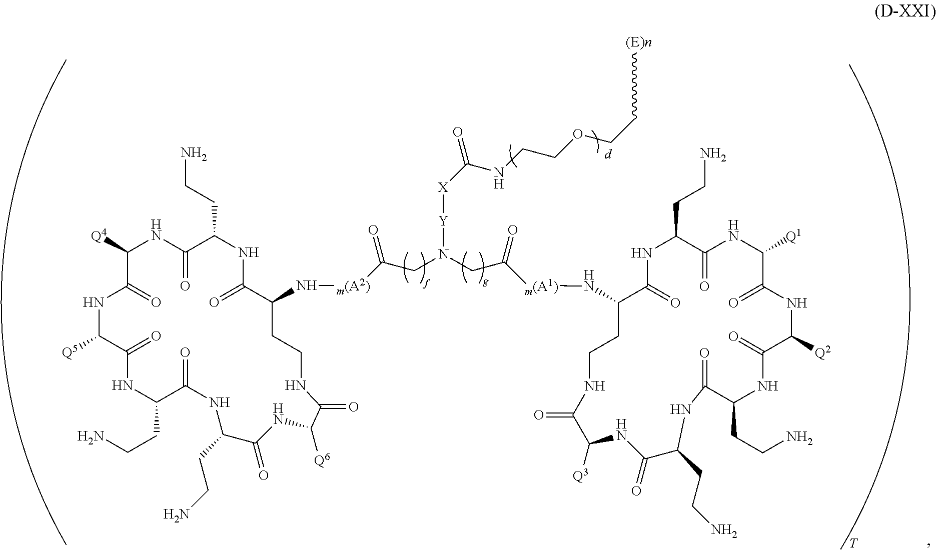

wherein: each A¹ and A² is an independently selected amino acid; each E is an Fc domain monomer; each Y is

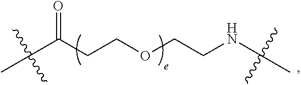

—C(O)CH₂CH₂—, —CH₂—, or is absent; each X is —C(O)CH₂CH₂— or is absent; each m is independently 0, 1, 2, 3, 4, or 5; n is 1 or 2; Q¹, Q², Q³, Q⁴, Q⁵ and Q⁶ are each independently selected from the side chain of an amino acid; each d is an integer from 0 to 15; each e is an integer from 1 to 10; each f is an integer from 1 to 5; each g is an integer from 1 to 5; and T is an integer from 1 to 20, e.g., from 1 to 15, or a pharmaceutically acceptable salt thereof.

In some embodiments of this aspect, each A¹ and A² is independently selected from glycine, arginine, asparagine, glutamine, 3-(2H-tetrazol-5-yl)alanine, 3-aminoalanine, piperazine-2-carboxylic acid, 2,4-diaminobutyic acid, 3-hydroxyproline, threonine, 2-amino-4-phenylbutyric acid, 3-(2-naphthyl)alanine, 2-piperazinecarboxylic acid, 2-aminooctanoic acid, serine, 2-aminohexanoic acid, 4-amino-4-piperidinyl carboxylic acid, methionine, methionine sulfoxide, methionine sulfone, S-methylcysteine, S-ethylcysteine, S-propylhomocysteine, cyclopropylalanine, 3-fluoroalanine, 2-amino-5-methylhexanoic acid, 2-amino-5-methylhex-4-enoic acid, alpha-t-butylglycine, and alpha-neopentylglycine; each m is 2, 3 or 4; each d is 1; when each Y is

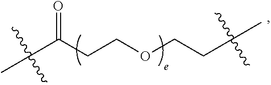

each e is 4; each f is 1 or 2; and each g is 1 or 2; or a pharmaceutically acceptable salt thereof.

In some embodiments of this aspect, each A¹ and A² is independently selected from 2,4-diaminobutyric acid, 3-aminoalanine, 2-piperazinecarboxylic acid, 2-aminohexanoic acid, 2-aminooctanoic acid, methionine, and threonine; each m is 2, 3 or 4: when each Y is

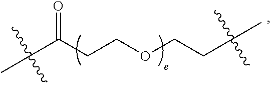

each e is 4; each d is 1; each f is 1 or 2; each g is 1 or 2; or a pharmaceutically acceptable salt thereof.

In some embodiments of this aspect, each A¹ and A² is independently selected from glycine, arginine, asparagine, glutamine, 3-(2H-tetrazol-5-yl)alanine, 3-aminoalanine, piperazine-2-carboxylic acid, 2,4-diaminobutyric acid, 3-hydroxyproline, threonine, 2-amino-4-phenylbutyric acid, 3-(2-naphthyl)alanine, 2-piperazinecarboxylic acid, 2-aminooctanoic acid, serine, 2-aminohexanoic acid, 4-amino-4-piperidinyl carboxylic acid, methionine, methionine sulfoxide, methionine sulfone, S-methylcysteine, S-ethylcysteine, S-propylhomocysteine, cyclopropylalanine, 3-fluoroalanine, 2-amino-5-methylhexanoic acid, 2-amino-5-methylhex-4-enoic acid, alpha-t-butylglycine, and alpha-neopentylglycine; each m is 3 or 4; each d is 1; each Y is absent; each f is 1; and each g is 1; or a pharmaceutically acceptable salt thereof.

In some embodiments of this aspect, each A¹ and A² is independently selected from 2,4-diaminobutyric acid and threonine; and each m is 3; or a pharmaceutically acceptable salt thereof.

In some embodiments of this aspect, each A¹ and A² is independently selected from glycine, arginine, asparagine, glutamine, 3-(2H-tetrazol-5-yl)alanine, 3-aminoalanine, piperazine-2-carboxylic acid, 2,4-diaminobutyric acid, 3-hydroxyproline, threonine, 2-amino-4-phenylbutyric acid, 3-(2-naphthyl)alanine, 2-piperazinecarboxylic acid, 2-aminooctanoic acid, serine, 2-aminohexanoic acid, 4-amino-4-piperidinyl carboxylic acid, methionine, methionine sulfoxide, methionine sulfone, S-methylcysteine, S-ethylcysteine, S-propylhomocysteine, cyclopropylalanine, 3-fluoroalanine, 2-amino-5-methylhexanoic acid, 2-amino-5-methylhex-4-enoic acid, alpha-t-butylglycine, and alpha-neopentylglycine; each m is 2, 3 or 4; each d is 1; each Y is —C(O)CH₂CH₂—; each f is 1 or 2; and each g is 1 or 2; or a pharmaceutically acceptable salt thereof.

In some embodiments of this aspect, each A¹ and A² is independently selected from glycine, arginine, asparagine, glutamine, 3-(2H-tetrazol-5-yl)alanine, 2,4-diaminobutyric acid, 3-aminoalanine, 2-aminohexanoic acid, 2-piperazinecarboxylic acid, 2-aminooctanoic acid, methionine, methionine sulfoxide, methionine sulfone, S-methylcysteine, S-ethylcysteine, S-propylhomocysteine, cyclopropylalanine, 3-fluoroalanine, 2-amino-5-methylhexanoic acid, 2-amino-5-methylhex-4-enoic acid, alpha-t-butylglycine, and alpha-neopentylglycine, or a pharmaceutically acceptable salt thereof.

In some embodiments, each m is 4, each d is 1, each f is 1, and each g is 1, or a pharmaceutically acceptable salt thereof. In some embodiments, each m is 3, each f is 2, and each g is 1, or a pharmaceutically acceptable salt thereof.

In some embodiments, each A¹ and A² is independently selected from glycine, arginine, asparagine, glutamine, 3-(2H-tetrazol-5-yl)alanine, 3-aminoalanine, piperazine-2-carboxylic acid, 2,4-diaminobutyric acid, 3-hydroxyproline, threonine, 2-amino-4-phenylbutyric acid, 3-(2-naphthyl)alanine, 2-piperazinecarboxylic acid, 2-aminooctanoic acid, serine, 2-aminohexanoic acid, 4-amino-4-piperidinyl carboxylic acid, methionine, methionine sulfoxide, methionine sulfone, S-methylcysteine, S-ethylcysteine, S-propylhomocysteine, cyclopropylalanine, 3-fluoroalanine, 2-amino-5-methylhexanoic acid, 2-amino-5-methylhex-4-enoic acid, alpha-t-butylglycine, and alpha-neopentylglycine; each m is 2, 3 or 4; each d is 1; each Y is —CH₂—; each f is 1 or 2; and each g is 1 or 2; or a pharmaceutically acceptable salt thereof.

In some embodiments of this aspect, each A¹ and A² is independently selected from 2,4-diaminobutyric acid, 2-aminooctanoic acid and threonine; each f is 1; and each g is 1; or a pharmaceutically acceptable salt thereof.

In some embodiments, each A¹ and A² is independently selected from 2,4-diaminobutyric acid, 2-aminohexanoic acid, 2-aminooctanoic acid and threonine, or a pharmaceutically acceptable salt thereof; each m is 4; each f is 1; and each g is 1; or a pharmaceutically acceptable salt thereof.

In some embodiments of this aspect, each A¹ and A² is independently selected from 2,4-diaminobutyric acid, 2-aminohexanoic acid, 2-aminooctanoic acid and threonine, or a pharmaceutically acceptable salt thereof; each m is 3; each f is 1; and each g is 1; or a pharmaceutically acceptable salt thereof.

In another aspect, the invention features a conjugate of formula (D-XXII):

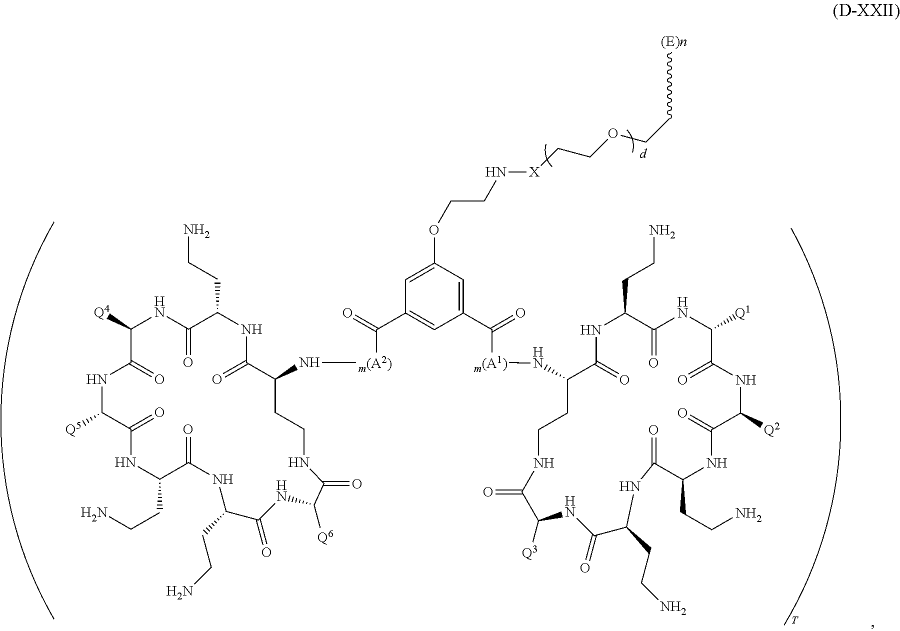

wherein: each A¹ and A² is an independently selected amino acid; each X is —C(O)CH₂CH₂CH₂—Y— or —C(O)CH₂CH₂C(O)NH—; each Y is heteroaryl; each E is an Fc domain monomer; each m is independently 0, 1, 2, 3, 4, or 5; n is 1 or 2; T is an integer from 1 to 20, e.g., from 1 to 15; Q¹, Q², Q³, Q⁴, Q⁵ and Q⁶ are each independently selected from the side chain of an amino acid; and each d is an integer from 0 to 15; or a pharmaceutically acceptable salt thereof.

In some embodiments of this aspect, each A¹ and A² is independently selected from glycine, arginine, asparagine, glutamine, 3-(2H-tetrazol-5-yl)alanine, 3-aminoalanine, piperazine-2-carboxylic acid, 2,4-diaminobutyric acid, 3-hydroxyproline, threonine, 2-amino-4-phenylbutyric acid, 3-(2-naphthyl)alanine, 2-piperazinecarboxylic acid, 2-aminooctanoic acid, serine, 2-aminohexanoic acid, 4-amino-4-piperidinyl carboxylic acid, methionine, methionine sulfoxide, methionine sulfone, S-methylcysteine, S-ethylcysteine, S-propylhomocysteine, cyclopropylalanine, 3-fluoroalanine, 2-amino-5-methylhexanoic acid, 2-amino-5-methylhex-4-enoic acid, alpha-t-butylglycine, and alpha-neopentylglycine; each m is 2 or 3; each d is an integer from 1 to 3; or a pharmaceutically acceptable salt thereof.

In some embodiments, each A¹ and A² is independently selected from 2,4-diaminobutyric acid, 3-(2-naphthyl)alanine, and threonine, or a pharmaceutically acceptable salt thereof.

In some embodiments, each X is —C(O)CH₂CH₂CH₂—Y—; each m is 3; each d is 3; and each Y is 1,4-triazololyl; or a pharmaceutically acceptable salt thereof.

In some embodiments, each X is —C(O)CH₂CH₂C(O)—; each m is 2; and each d is 1; or a pharmaceutically acceptable salt thereof.

In another aspect, the invention features a conjugate of formula (D-XXIII):

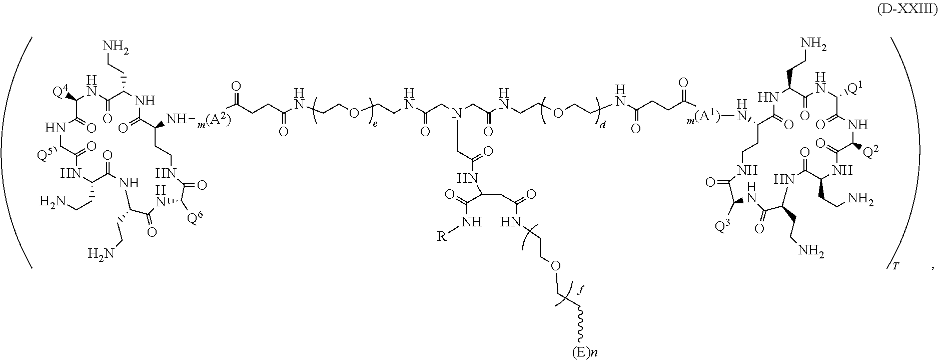

wherein: each A¹ and A² is an independently selected amino acid; each E is an Fc domain monomer, each R is C1-C20 alkyl; each m is independently 0, 1, 2, 3, 4, or 5; n is 1 or 2; Q¹, Q², Q³, Q⁴, Q⁵ and Q⁶ are each independently selected from the side chain of an amino acid; each d is an integer from 0 to 20; each e is an integer from 0 to 20; each f is an integer from 0 to 20: and T is an integer from 1 to 20, e.g., from 1 to 15, or a pharmaceutically acceptable salt thereof.

In some embodiments of this aspect, each A¹ and A² is independently selected from glycine, arginine, asparagine, glutamine, 3-(2H-tetrazol-5-yl)alanine, 3-aminoalanine, piperazine-2-carboxylic acid, 2,4-diaminobutyic acid, 3-hydroxyproline, threonine, 2-amino-4-phenylbutyric acid, 3-(2-naphthyl)alanine, 2-piperazinecarboxylic acid, 2-aminooctanoic acid, serine, 2-aminohexanoic acid, 4-amino-4-piperidinyl carboxylic acid, methionine, methionine sulfoxide, methionine sulfone, S-methylcysteine, S-ethylcysteine, S-propylhomocysteine, cyclopropylalanine, 3-fluoroalanine, 2-amino-5-methylhexanoic acid, 2-amino-5-methylhex-4-enoic acid, alpha-t-butylglycine, and alpha-neopentylglycine; each m is 2 or 3; each d is an integer from 1 to 3; each e is an integer from 1 to 3; each f is an integer from 1 to 3; and each R is C1-C10 alkyl; or a pharmaceutically acceptable salt thereof.

In some embodiments, each A¹ and A² is independently selected from 2,4-diaminobutyic acid and threonine; each m is 2; each d is 1; each e is 1; each f is 1; each R is C1-C10 alkyl; and or a pharmaceutically acceptable salt thereof.

In another aspect a conjugate of formula (D-XXIV):

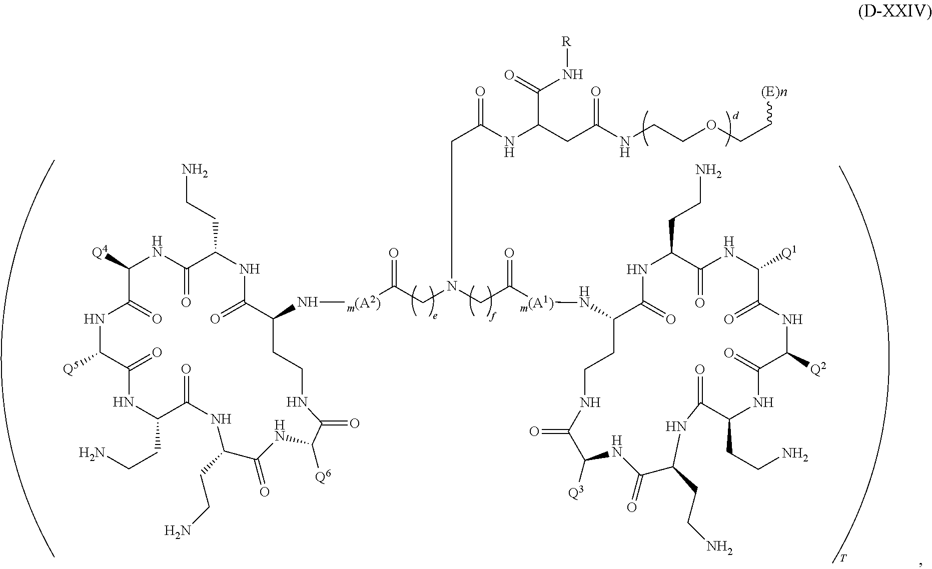

wherein: each A¹ and A² is an independently selected amino acid; each E is an Fc domain monomer; each R is C1-C20 alkyl; each m is independently 0, 1, 2, 3, 4, or 5; n is 1 or 2; Q¹, Q², Q³, Q⁴, Q⁵ and Q⁶ are each independently selected from the side chain of an amino acid; each d is an integer from 1 to 20; each e is an integer from 1 to 20; each f is an integer from 1 to 20; and T is an integer from 1 to 20, e.g., from 1 to 15, or a pharmaceutically acceptable salt thereof.

In some embodiments of this aspect, each A¹ and A² is independently selected from glycine, arginine, asparagine, glutamine, 3-(2H-tetrazol-5-yl)alanine, 3-aminoalanine, piperazine-2-carboxylic acid, 2,4-diaminobutyric acid, 3-hydroxyproline, threonine, 2-amino-4-phenylbutyric acid, 3-(2-naphthyl)alanine, 2-piperazinecarboxylic acid, 2-aminooctanoic acid, serine, 2-aminohexanoic acid, 4-amino-4-piperidinyl carboxylic acid, methionine, methionine sulfoxide, methionine sulfone. S-methylcysteine, S-ethylcysteine, S-propylhomocysteine, cyclopropylalanine, 3-fluoroalanine, 2-amino-5-methylhexanoic acid, 2-amino-5-methylhex-4-enoic acid, alpha-t-butylglycine, and alpha-neopentylglycine; each m is 2, 3, or 4; each d is an integer from 1 to 3; each e is an integer from 1 to 3; each f is an integer from 1 to 3; each R is C1-C10 alkyl; and or a pharmaceutically acceptable salt thereof.

In some embodiments, each A¹ and A² is independently selected from 2,4-diaminobutyic acid, 2-aminooctanoic acid, and threonine; each m is 2, 3, or 4; each d is 1; each e is 1; each f is 1; and each R is C1-C10 alkyl; or a pharmaceutically acceptable salt thereof.

In some embodiments, each m is 2, or a pharmaceutically acceptable salt thereof. In some embodiments, each m is 3, or a pharmaceutically acceptable salt thereof. In some embodiments, each m is 4, or a pharmaceutically acceptable salt thereof.

In another aspect, the invention features a conjugate of formula (D-XXV):

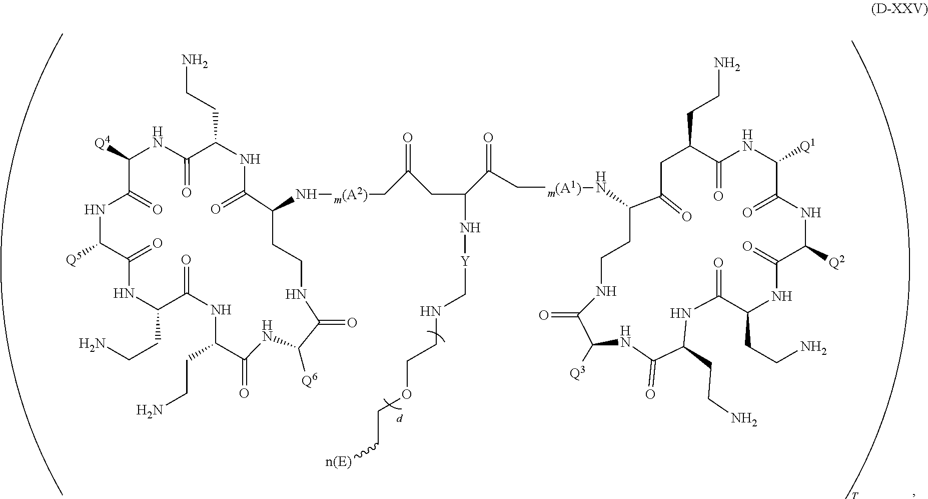

wherein: each A¹ and A² is an independently selected amino acid; each E is an Fc domain monomer; each Y is

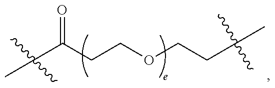

—C(O)CH₂CH₂—, —CH₂—, or is absent; each e is an integer from 1 to 10; each m is independently 0, 1, 2, 3, 4, or 5; n is 1 or 2; Q¹, Q², Q³, Q⁴, Q⁵ and Q⁶ are each independently selected from the side chain of an amino acid; each d is an integer from 0 to 15; and T is an integer from 1 to 20, e.g., from 1 to 15, or a pharmaceutically acceptable salt thereof.

In some embodiments of this aspect, each Y is —C(O)CH₂CH₂—, or a pharmaceutically acceptable salt thereof.

In some embodiments of this aspect, each A¹ and A² is independently selected from glycine, arginine, asparagine, glutamine, 3-(2H-tetrazol-5-yl)alanine, 3-aminoalanine, piperazine-2-carboxylic acid, 2,4-diaminobutyic acid, 3-hydroxyproline, threonine, 2-amino-4-phenylbutyric acid, 3-(2-naphthyl)alanine, 2-piperazinecarboxylic acid, 2-aminooctanoic acid, serine, 2-aminohexanoic acid, 4-amino-4-piperidinyl carboxylic acid, methionine, methionine sulfoxide, methionine sulfone, S-methylcysteine, S-ethylcysteine, S-propylhomocysteine, cyclopropylalanine, 3-fluoroalanine, 2-amino-5-methylhexanoic acid, 2-amino-5-methylhex-4-enoic acid, alpha-t-butylglycine, and alpha-neopentylglycine; each m is 2, 3, or 4; and each d is an integer from 1 to 3; or a pharmaceutically acceptable salt thereof.

In some embodiments, each A¹ and A² is independently selected from 2,4-diaminobutyic acid, 2-aminooctanoic acid, and threonine; and each d is 1; or a pharmaceutically acceptable salt thereof.

In some embodiments, each m is 2, or a pharmaceutically acceptable salt thereof. In some embodiments, each m is 3, or a pharmaceutically acceptable salt thereof. In some embodiments, each m is 4, or a pharmaceutically acceptable salt thereof.

In another aspect, the invention features a conjugate of formula (D-XXVI):

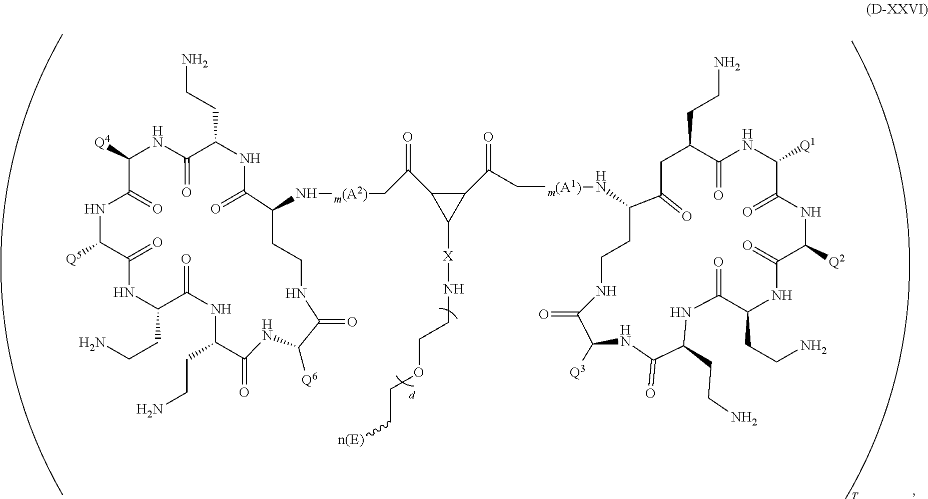

wherein: each A¹ and A² is an independently selected amino acid; each E is an Fc domain monomer; each X is —CH₂— or —C(O)—; each m is independently 0, 1, 2, 3, 4, or 5; n is 1 or 2; Q¹, Q², Q³, Q⁴, Q⁵ and Q⁶ are each independently selected from the side chain of an amino acid; each d is an integer from 0 to 15; and T is an integer from 1 to 20, e.g., from 1 to 15, or a pharmaceutically acceptable salt thereof.

In some embodiments of this aspect, each A¹ and A² is independently selected from glycine, arginine, asparagine, glutamine, 3-(2H-tetrazol-5-yl)alanine, 3-aminoalanine, piperazine-2-carboxylic acid, 2,4-diaminobutyic acid, 3-hydroxyproline, threonine, 2-amino-4-phenylbutyric acid, 3-(2-naphthyl)alanine, 2-piperazinecarboxylic acid, 2-aminooctanoic acid, serine, 2-aminohexanoic acid, 4-amino-4-piperidinyl carboxylic acid, methionine, methionine sulfoxide, methionine sulfone, S-methylcysteine, S-ethylcysteine, S-propylhomocysteine, cyclopropylalanine, 3-fluoroalanine, 2-amino-5-methylhexanoic acid, 2-amino-5-methylhex-4-enoic acid, alpha-t-butylglycine, and alpha-neopentylglycine; each m is 2, 3, or 4; and each d is an integer from 1 to 3; or a pharmaceutically acceptable salt thereof.

In some embodiments, each A¹ and A² is independently selected from 2,4-diaminobutyic acid, 2-aminooctanoic acid, and threonine; and each d is 1; or a pharmaceutically acceptable salt thereof.

In some embodiments, each m is 2, or a pharmaceutically acceptable salt thereof. In some embodiments, each m is 3, or a pharmaceutically acceptable salt thereof. In some embodiments, each m is 4, or a pharmaceutically acceptable salt thereof.

In another aspect, the invention features a conjugate of formula (D-XXVII):

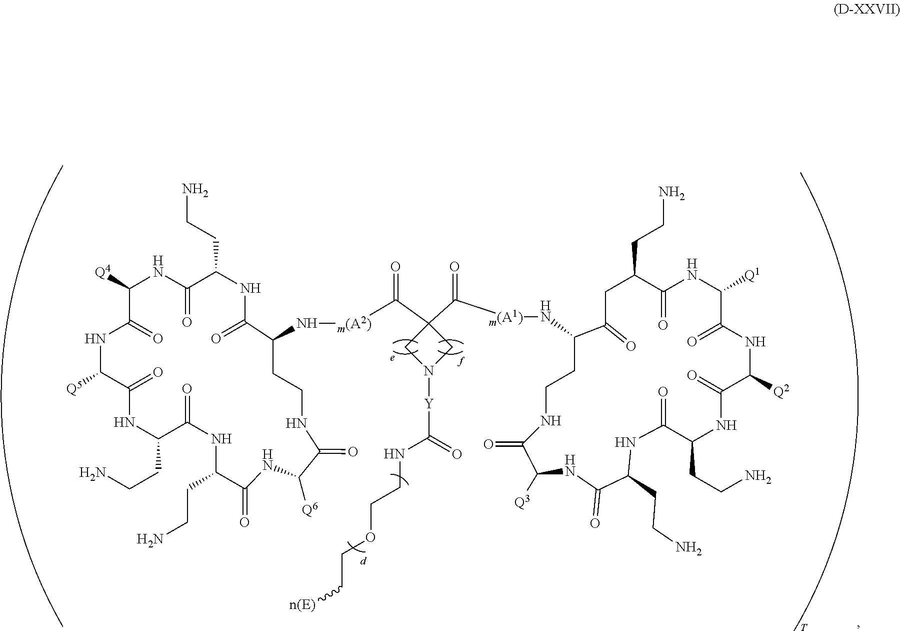

wherein: each A¹ and A² is an independently selected amino acid; each E is an Fc domain monomer; each Y is

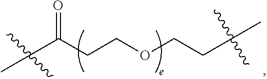

—C(O)CH₂CH₂—, —CH₂—, or is absent; each m is independently 0, 1, 2, 3, 4, or 5; n is 1 or 2; Q¹, Q², Q³, Q⁴, Q⁵ and Q⁶ are each independently selected from the side chain of an amino acid; each d is an integer from 0 to 15; each e is an integer from 1 to 3; each f is an integer from 1 to 3; and T is an integer from 1 to 20, e.g., from 1 to 15, or a pharmaceutically acceptable salt thereof.

In some embodiments of this aspect, each Y is —C(O)CH₂CH₂—, or a pharmaceutically acceptable salt thereof.

In some embodiments of this aspect, each A¹ and A² is independently selected from glycine, arginine, asparagine, glutamine, 3-(2H-tetrazol-5-yl)alanine, 3-aminoalanine, piperazine-2-carboxylic acid, 2,4-diaminobutyic acid, 3-hydroxyproline, threonine, 2-amino-4-phenylbutyric acid, 3-(2-naphthyl)alanine, 2-piperazinecarboxylic acid, 2-aminooctanoic acid, serine, 2-aminohexanoic acid, 4-amino-4-piperidinyl carboxylic acid, methionine, methionine sulfoxide, methionine sulfone, S-methylcysteine, S-ethylcysteine, S-propylhomocysteine, cyclopropylalanine, 3-fluoroalanine, 2-amino-5-methylhexanoic acid, 2-amino-5-methylhex-4-enoic acid, alpha-t-butylglycine, and alpha-neopentylglycine; each m is 2, 3, or 4; each d is an integer from 1 to 3; or a pharmaceutically acceptable salt thereof.

In some embodiments, each A¹ and A² is independently selected from 2,4-diaminobutyic acid, 2-aminooctanoic acid, and threonine; and each d is 1; and each e is 1 or 2; or a pharmaceutically acceptable salt thereof.

In some embodiments, each m is 2, or a pharmaceutically acceptable salt thereof. In some embodiments, each m is 3, or a pharmaceutically acceptable salt thereof. In some embodiments, each m is 4, or a pharmaceutically acceptable salt thereof.

In another aspect, the invention features a conjugate of formula (D-XXVIII):

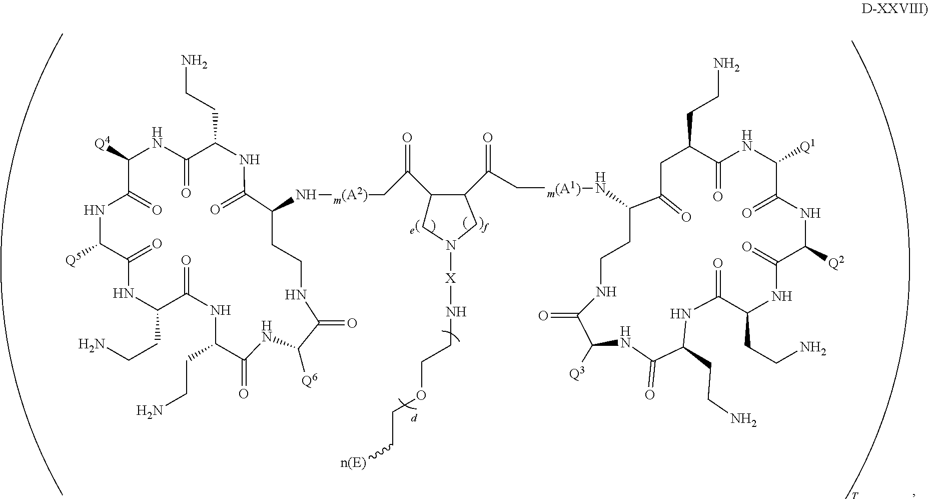

wherein: each A¹ and A² is an independently selected amino acid; each E is an Fc domain monomer; each X is independently selected from

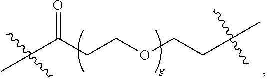

—C(O)CH₂CH₂C(O)—, —CH₂CH₂NHC(O)CH₂CH₂—, —C(O)—, and —CH₂—, or is absent; each m is independently 0, 1, 2, 3, 4, or 5; n is 1 or 2; Q¹, Q², Q³, Q⁴, Q⁵ and Q⁶ are each independently selected from the side chain of an amino acid; each d is an integer from 1 to 15; each g is independently an integer from 1 to 15; each e is independently from 1 to 15; each f is independently an integer from 1 to 15; and T is an integer from 1 to 20, e.g., from 1 to 15, or a pharmaceutically acceptable salt thereof.

In some embodiments of this aspect, the conjugate is of the formula (D-XXVIII-1):

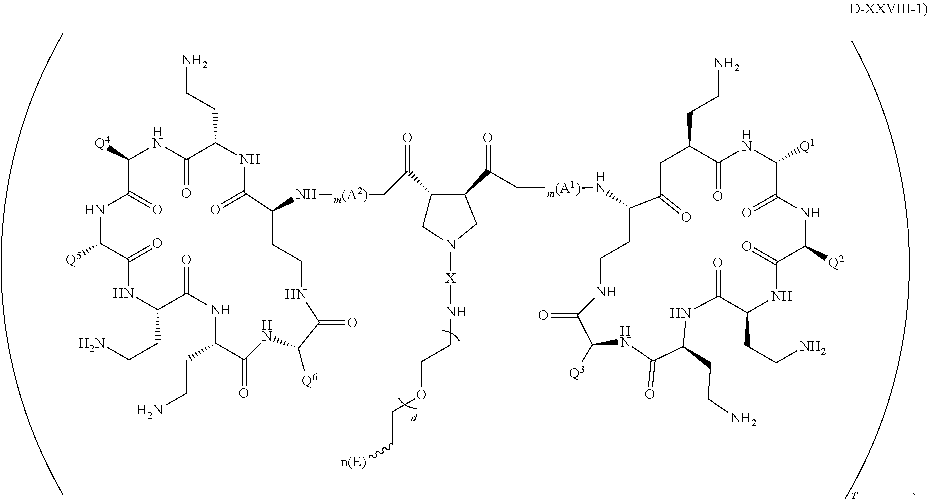

or a pharmaceutically acceptable salt thereof.

In some embodiments, the conjugate is of the formula (D-XXVIII-2):

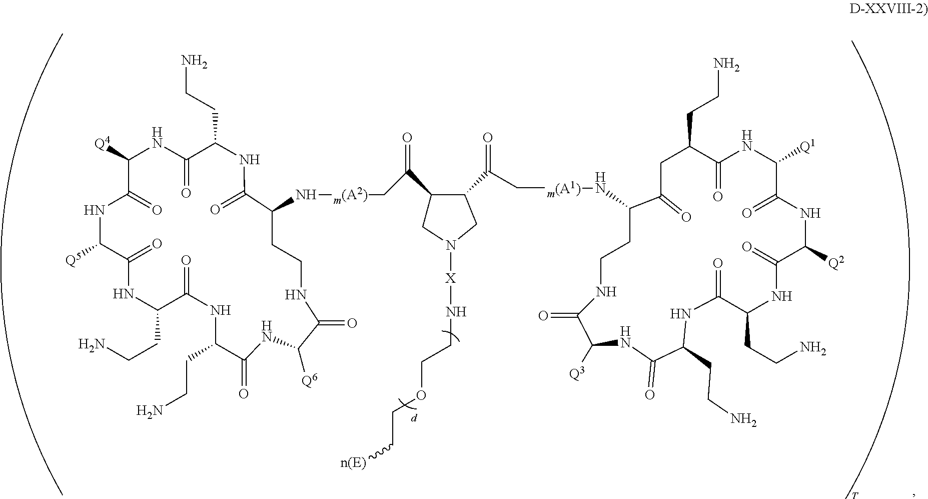

or a pharmaceutically acceptable salt thereof.

In each A¹ and A² is independently selected from glycine, arginine, asparagine, glutamine, 3-(2H-tetrazol-5-yl)alanine, 3-aminoalanine, piperazine-2-carboxylic acid, 2,4-diaminobutyric acid, 3-hydroxyproline, threonine, 2-amino-4-phenylbutyric acid, 3-(2-naphthyl)alanine, 2-piperazinecarboxylic acid, 2-aminooctanoic acid, serine, 2-aminohexanoic acid, 4-amino-4-piperidinyl carboxylic acid, methionine, methionine sulfoxide, methionine sulfone, S-methylcysteine, S-ethylcysteine, S-propylhomocysteine, cyclopropylalanine, 3-fluoroalanine, 2-amino-5-methylhexanoic acid, 2-amino-5-methylhex-4-enoic acid, alpha-t-butylglycine, and alpha-neopentylglycine; each m is 2, 3, or 4; each d is an integer from 1 to 5; each e is 1; each X is —C(O)CH₂CH₂C(O)—; or a pharmaceutically acceptable salt thereof.

In some embodiments of this aspect, each A¹ and A² is independently selected from 2,4-diaminobutyric acid, 2-aminohexanoic acid, and threonine; and each d is 1; or a pharmaceutically acceptable salt thereof.

In some embodiments, each m is 2, or a pharmaceutically acceptable salt thereof. In some embodiments, each m is 3, or a pharmaceutically acceptable salt thereof. In some embodiments, each m is 4, or a pharmaceutically acceptable salt thereof.

In some embodiments of the conjugates described herein, Q¹, Q², Q³, Q⁴, Q⁵ and Q⁶ are each independently selected from the side chain of a natural amino acid, or a pharmaceutically acceptable salt thereof. In some embodiments, at least one of Q¹, Q², Q³, Q⁴, Q⁵ and Q⁶ is selected from the side chain of a non-natural amino acid, or a pharmaceutically acceptable salt thereof. In some embodiments, at least two of Q¹, Q², Q³, Q⁴, Q⁵ and Q⁶ are independently selected from the side chain of a non-natural amino acid, or a pharmaceutically acceptable salt thereof. In some embodiments, at least three of Q¹, Q², Q³, Q⁴, Q⁵ and Q⁶ are independently selected from the side chain of a non-natural amino acid, or a pharmaceutically acceptable salt thereof. In some embodiments, at least four of Q¹, Q², Q³, Q⁴, Q⁵ and Q⁶ are independently selected from the side chain of a non-natural amino acid, or a pharmaceutically acceptable salt thereof. In some embodiments, at least five of Q¹, Q², Q³, Q⁴, Q⁵ and Q⁶ are independently selected from the side chain of a non-natural amino acid, or a pharmaceutically acceptable salt thereof. In some embodiments, each of Q¹, Q², Q³, Q⁴, Q⁵ and Q⁶ is independently selected from the side chain of a non-natural amino acid, or a pharmaceutically acceptable salt thereof.

In some embodiments, each Q¹, Q², Q³, Q⁴, Q⁵ and Q⁶ is independently selected from the side chain of serine, threonine, cysteine, glycine, proline, alanine, valine, isoleucine, leucine, methionine, phenylalanine, tyrosine, and tryptophan, or a pharmaceutically acceptable salt thereof.

In some embodiments, each of Q¹, Q², Q³, Q⁴, Q⁵ and Q⁶ is independently selected from C1-C4 alkyl, C1-C2 hydroxyalkyl, C1-C5 alkamino, and C6-C35 alkaryl, or a pharmaceutically acceptable salt thereof.

In some embodiments, each of Q¹, Q², Q³, Q⁴, Q⁵ and Q⁶ is independently selected from 2-methyl-1-propyl, 2-propyl, 1-hydroxyethyl, butyl, benzyl, hydroxymethyl, propyl, 2-butyl, methyl, and 2-aminoethyl, or a pharmaceutically acceptable salt thereof.

In some embodiments, the combination of Q¹, Q², Q³, Q⁴, Q⁵ and Q⁶ is selected from one of

| Q¹ | Q² | Q³ | Q⁴ | Q⁵ | Q⁶ |
|---|---|---|---|---|---|
| 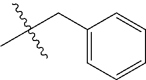 [SMILES: *Cc1ccccc1] | 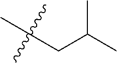 [SMILES: *CC(C)C] | 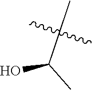 [SMILES: *[C@@H](C)O] | 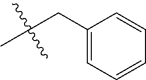 [SMILES: *Cc1ccccc1] | 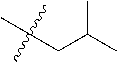 [SMILES: *CC(C)C] | 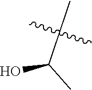 [SMILES: *[C@@H](C)O] |
| 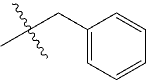 [SMILES: *Cc1ccccc1] | 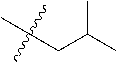 [SMILES: *CC(C)C] | 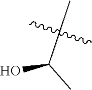 [SMILES: *[C@@H](C)O] | 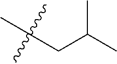 [SMILES: *CC(C)C] | 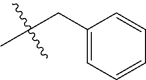 [SMILES: *Cc1ccccc1] | 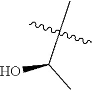 [SMILES: *[C@@H](C)O] |
| 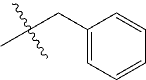 [SMILES: *Cc1ccccc1] | 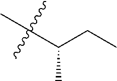 [SMILES: *[C@@H](C)CC] | 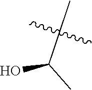 [SMILES: *[C@@H](C)O] | 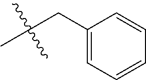 [SMILES: *Cc1ccccc1] | 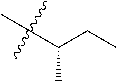 [SMILES: *[C@@H](C)CC] | 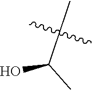 [SMILES: *[C@@H](C)O] |
| 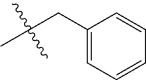 [SMILES: *Cc1ccccc1] | 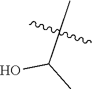 [SMILES: *C(C)O] | 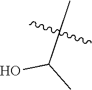 [SMILES: *C(C)O] | 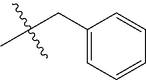 [SMILES: *Cc1ccccc1] | 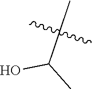 [SMILES: *C(C)O] | 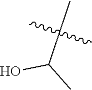 [SMILES: *C(C)O] |
| 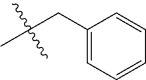 [SMILES: *Cc1ccccc1] | 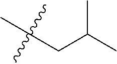 [SMILES: *CC(C)C] | 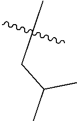 [SMILES: *CC(C)C] | 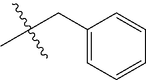 [SMILES: *Cc1ccccc1] | 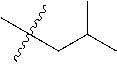 [SMILES: *CC(C)C] | 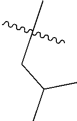 [SMILES: *CC(C)C] |
| 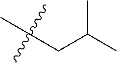 [SMILES: *CC(C)C] | 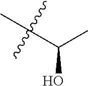 [SMILES: *[C@@H](C)O] | 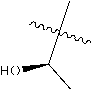 [SMILES: *[C@@H](C)O] | 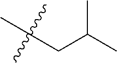 [SMILES: *CC(C)C] | 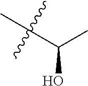 [SMILES: *[C@@H](C)O] | 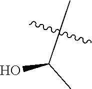 [SMILES: *[C@@H](C)O] |
| 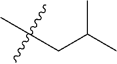 [SMILES: *CC(C)C] | 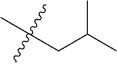 [SMILES: *CC(C)C] | 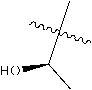 [SMILES: *[C@@H](C)O] | 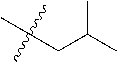 [SMILES: *CC(C)C] | 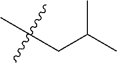 [SMILES: *CC(C)C] | 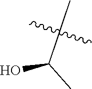 [SMILES: *[C@@H](C)O] |
| 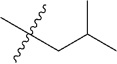 [SMILES: *CC(C)C] | 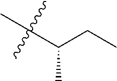 [SMILES: *[C@@H](C)CC] | 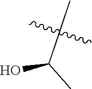 [SMILES: *[C@@H](C)O] | 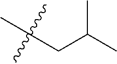 [SMILES: *CC(C)C] | 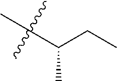 [SMILES: *[C@@H](C)CC] | 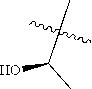 [SMILES: *[C@@H](C)O] |
| 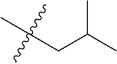 [SMILES: *CC(C)C] | 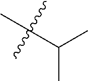 [SMILES: *C(C)C] | 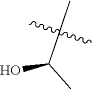 [SMILES: *[C@@H](C)O] | 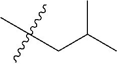 [SMILES: *CC(C)C] | 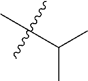 [SMILES: *C(C)C] | 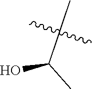 [SMILES: *[C@@H](C)O] |
| 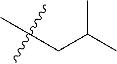 [SMILES: *CC(C)C] | 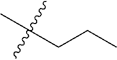 [SMILES: *CCC] | 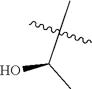 [SMILES: *[C@@H](C)O] | 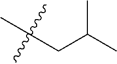 [SMILES: *CC(C)C] | 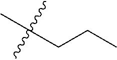 [SMILES: *CCC] | 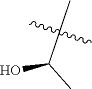 [SMILES: *[C@@H](C)O] |
| 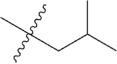 [SMILES: *CC(C)C] | —CH₃ | 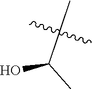 [SMILES: *[C@@H](C)O] | 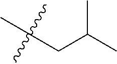 [SMILES: *CC(C)C] | —CH₃ | 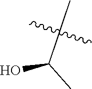 [SMILES: *[C@@H](C)O] |
| 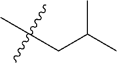 [SMILES: *CC(C)C] | —CH₂OH | 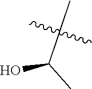 [SMILES: *[C@@H](C)O] | 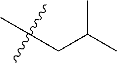 [SMILES: *CC(C)C] | —CH₂OH | 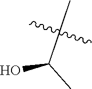 [SMILES: *[C@@H](C)O] |
| 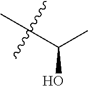 [SMILES: *[C@@H](C)O] | 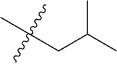 [SMILES: *CC(C)C] | 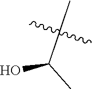 [SMILES: *[C@@H](C)O] | 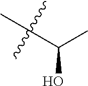 [SMILES: *[C@@H](C)O] | 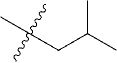 [SMILES: *CC(C)C] | 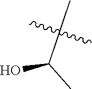 [SMILES: *[C@@H](C)O] |
| 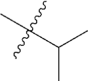 [SMILES: *C(C)C] | 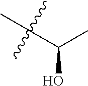 [SMILES: *[C@@H](C)O] | 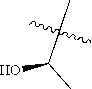 [SMILES: *[C@@H](C)O] | 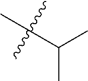 [SMILES: *C(C)C] | 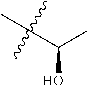 [SMILES: *[C@@H](C)O] | 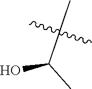 [SMILES: *[C@@H](C)O] |
| 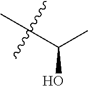 [SMILES: *[C@@H](C)O] | 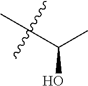 [SMILES: *[C@@H](C)O] | 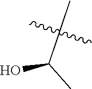 [SMILES: *[C@@H](C)O] | 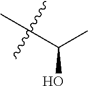 [SMILES: *[C@@H](C)O] | 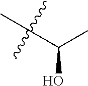 [SMILES: *[C@@H](C)O] | 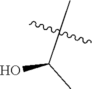 [SMILES: *[C@@H](C)O] |
| 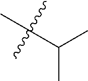 [SMILES: *C(C)C] | 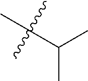 [SMILES: *C(C)C] | 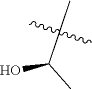 [SMILES: *[C@@H](C)O] | 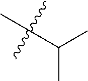 [SMILES: *C(C)C] | 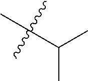 [SMILES: *C(C)C] | 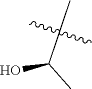 [SMILES: *[C@@H](C)O] |
| 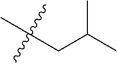 [SMILES: *CC(C)C] | —CH₂CH₂NH₂ | 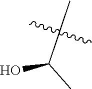 [SMILES: *[C@@H](C)O] | 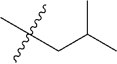 [SMILES: *CC(C)C] | —CH₂CH₂NH₂ | 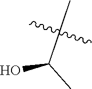 [SMILES: *[C@@H](C)O] |

or a pharmaceutically acceptable salt thereof.

In another aspect, the invention features a conjugate described by formula (M-Ia):

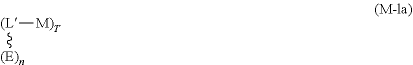

wherein each M includes a cyclic heptapeptide including a linking nitrogen atom; each E is an Fc domain monomer, L′ in each L′-M is a linker, wherein L′ is covalently attached to the linking nitrogen atom in M and to E; n is 1 or 2; and T is an integer from 1 to 20, e.g., from 1 to 15; or a pharmaceutically acceptable salt thereof.

In some embodiments, when n is 2, E dimerizes to form an Fc domain, and the conjugate is described by formula (M-Ib):

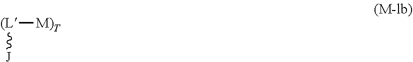

wherein J is an Fc domain; and T is an integer from 2 to 20, e.g., 2 to 15, or a pharmaceutically acceptable salt thereof.

In some embodiments. L¹ in each L′-M is described by formula (M-L′):

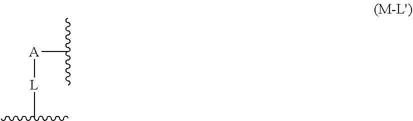

wherein L is a remainder of L′; and A is 1 to b amino acids, wherein at least one A is covalently attached to the linking nitrogen atom in M, or a pharmaceutically acceptable salt thereof.

In some embodiments, the conjugate is described by formula (M-II):

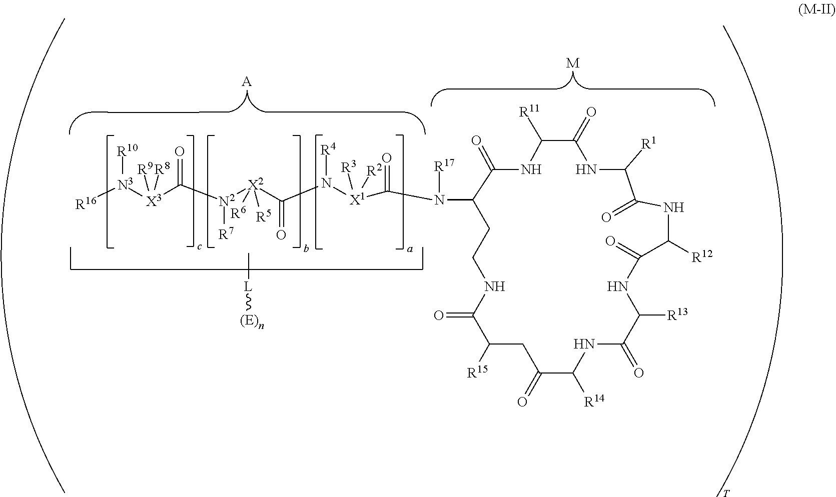

wherein each L is a remainder of L′ and is covalently attached to or may take the place of R², R³, R⁴, R⁵, R⁶, R⁷, R⁸, R⁹, R¹⁰, or R¹⁶; each of R¹ and R¹² is, independently, a lipophilic moiety, a polar moiety, or H; each of R¹¹, R¹³, and R¹⁴ is, independently, optionally substituted C1-C5 alkamino, a polar moiety, a positively charged moiety, or H; each R¹⁵ is a lipophilic moiety or a polar moiety; each of R², R³, R⁴, R⁵, R⁶, R⁷, R⁸, R⁹, and R¹⁰ is, independently, a lipophilic moiety, a positively charged moiety, a polar moiety, H, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; or two R groups on the same or adjacent atoms join to form an optionally substituted 3-8 membered ring (e.g., 5-8 membered ring); each R¹⁶ is H, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or is absent; R¹⁷ is H, C1-C3 alkyl, or C2-C3 alkamino; each of a, b, and c is, independently, 0 or 1; and each of X¹, X², and X³ is a carbon atom or a nitrogen atom, wherein if X¹ is a nitrogen atom then R³ is absent, if X² is a nitrogen atom then R⁵ is absent, and if X³ is a nitrogen atom then R⁹ is absent; or a pharmaceutically acceptable salt thereof.

In some embodiments, the conjugate includes at least one optionally substituted 3-8 membered ring (e.g., 5-8 membered ring) formed by joining (i) R², R³, and X¹; (ii) R³, R⁴, N¹, and X¹; (iii) R⁵, R⁶, and X²; (iv) R⁶, R⁷, N², and X²; (v) R⁸, R⁹, and X³; or (vi) R⁹, R¹⁰, N³, and X³, or a pharmaceutically acceptable salt thereof.

In some embodiments of this aspect, the conjugate is described by formula (M-IIa):

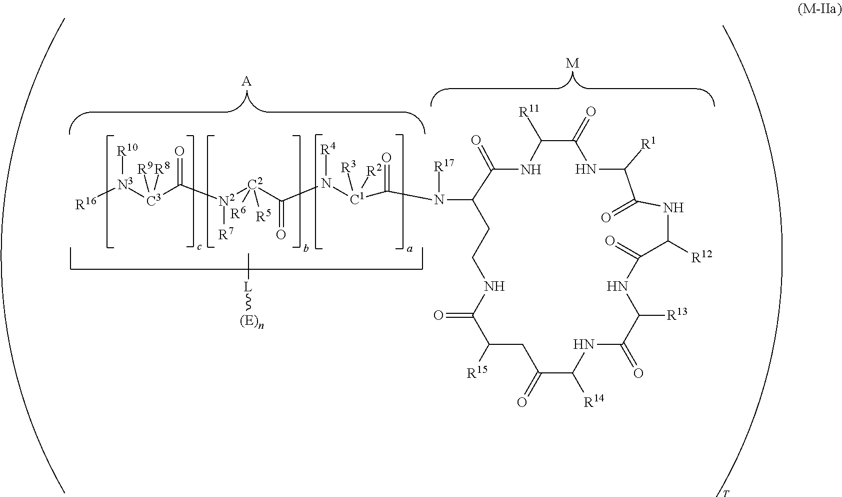

wherein each L is a remainder of L′ and is covalently attached to or may take the place of R², R³, R⁴, R⁵, R⁶, R⁷, R⁸, R⁹, R¹⁰, or R¹⁶; each of R¹ and R¹² is, independently, a lipophilic moiety, a polar moiety, or H; each of R¹¹, R¹³, and R¹⁴ is, independently, optionally substituted C1-C5 alkamino, a polar moiety, a positively charged moiety, or H; each R¹⁵ is a lipophilic moiety or a polar moiety; each of R², R³, R⁴, R⁵, R⁶, R⁷, R⁸, R⁹, and R¹⁰ is, independently, a lipophilic moiety, a positively charged moiety, a polar moiety, H, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; or two R groups on the same or adjacent atoms join to form an optionally substituted 3-8 membered ring (e.g., 5-8 membered ring); each R¹⁶ is H, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or is absent; R¹⁷ is H, C1-C3 alkyl, or C2-C3 alkamino; and each of a, b, and c is, independently, 0 or 1; or a pharmaceutically acceptable salt thereof.

In some embodiments of this aspect, the conjugate includes at least one optionally substituted 3-8 membered ring (e.g., 5-8 membered ring) formed by joining (i) R², R³, and C¹; (ii) R³, R⁴, N¹, and C¹; (iii) R⁵, R⁶, and C²; (iv) R⁸, R⁷, N², and C²; (v) R⁸, R⁹, and C³; or (vi) R⁹, R¹⁰, N³, and C³, or a pharmaceutically acceptable salt thereof.

In some embodiments, the conjugate is described by formula (M-IIb):

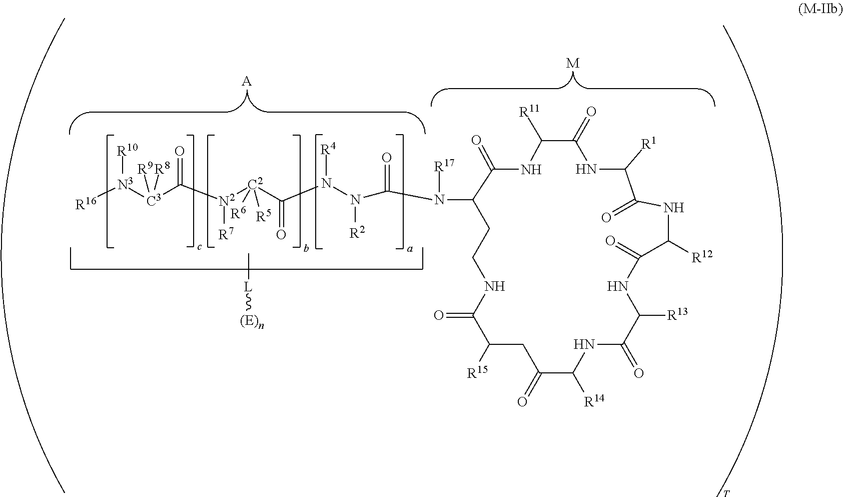

wherein each L is a remainder of L′ and is covalently attached to or may take the place of R², R⁴, R⁵, R⁶, R⁷, R⁸, R⁹, R¹⁰, or R¹⁶; each of R¹ and R¹² is, independently, a lipophilic moiety, a polar moiety, or H; each of R¹¹, R¹³, and R¹⁴ is, independently, optionally substituted C1-C5 alkamino, a polar moiety, a positively charged moiety, or H; each R¹⁵ is a lipophilic moiety or a polar moiety; each of R², R³, R⁴, R⁵, R⁶, R⁷, R⁸, R⁹, and R¹⁰ is, independently, hydrogen, a lipophilic moiety, a positively charged moiety, a polar moiety, H, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; or two R groups on the same or adjacent atoms join to form an optionally substituted 3-8 membered ring (e.g., 5-8 membered ring); each R¹⁶ is H, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or is absent; R¹⁷ is H, C1-C3 alkyl, or C2-C3 alkamino; and each of a, b, and c is, independently, 0 or 1; or a pharmaceutically acceptable salt thereof.

In some embodiments, the conjugate is described by formula (M-IIc):

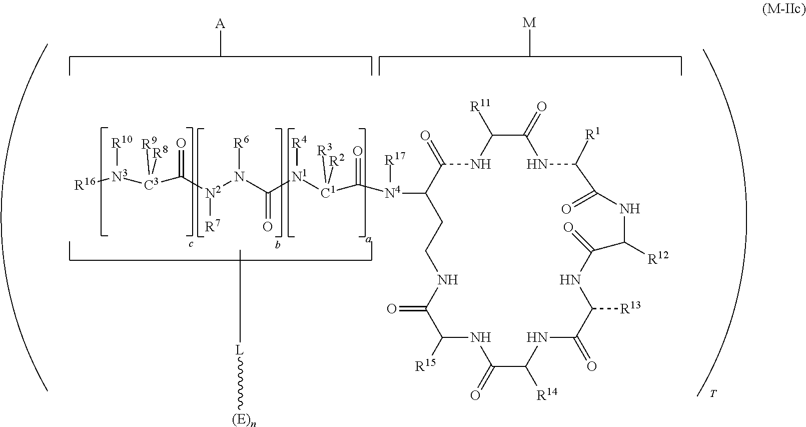

wherein each L is a remainder of L′ and is covalently attached to or may take the place of R², R³, R⁴, R⁵, R⁶, R⁷, R⁸, R⁹, R¹⁰, or R¹⁸; each of R¹ and R¹² is, independently, a lipophilic moiety, a polar moiety, or H; each of R¹¹, R¹³, and R¹⁴ is, independently, optionally substituted C1-C5 alkamino, a polar moiety, a positively charged moiety, or H; each R¹⁵ is a lipophilic moiety or a polar moiety; each of R², R³, R⁴, R⁵, R⁷, R⁸, R⁹, and R¹⁰ is, independently, a lipophilic moiety, a positively charged moiety, a polar moiety, H, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; or two R groups on the same or adjacent atoms join to form an optionally substituted 3-8 membered ring (e.g., 5-8 membered ring); each R¹⁸ is H, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or is absent; R¹⁷ is H, C1-C3 alkyl, or C2-C3 alkamino; and each of a, b, and c is, independently, 0 or 1; or a pharmaceutically acceptable salt thereof.

In some embodiments, the conjugate is described by formula (M-IId):

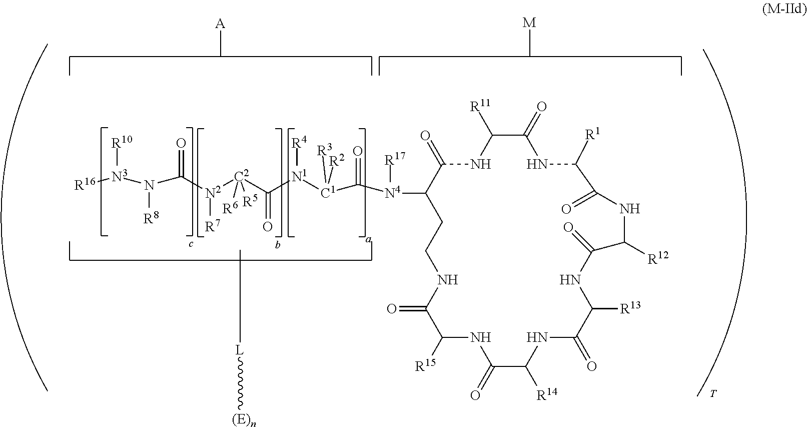

wherein each L is a remainder of L′ and is covalently attached to or may take the place of R², R³, R⁴, R⁵, R⁶, R⁷, R⁸, R⁹, R¹⁰, or R¹⁶; each of R¹ and R¹² is, independently, a lipophilic moiety, a polar moiety, or H; each of R¹¹, R¹³, and R¹⁴ is, independently, optionally substituted C1-C5 alkamino, a polar moiety, a positively charged moiety, or H; each R's is a lipophilic moiety or a polar moiety; each of R², R³, R⁴, R⁵, R⁶, R⁷, R⁸, and R¹⁰ is, independently, a lipophilic moiety, a positively charged moiety, a polar moiety, H, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; or two R groups on the same or adjacent atoms join to form an optionally substituted 3-8 membered ring (e.g., 5-8 membered ring); each R¹⁸ is H, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or is absent; R¹⁷ is H, C1-C3 alkyl, or C2-C3 alkamino; and each of a, b, and c is, independently, 0 or 1; or a pharmaceutically acceptable salt thereof.

In some embodiments, the conjugate is described by formula (M-IIIa):

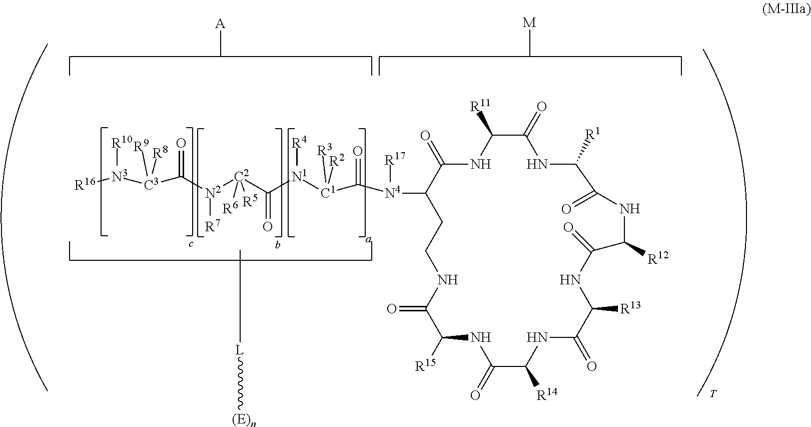

or a pharmaceutically acceptable salt thereof.

In some embodiments, the conjugate is described by formula (M-IIIb):

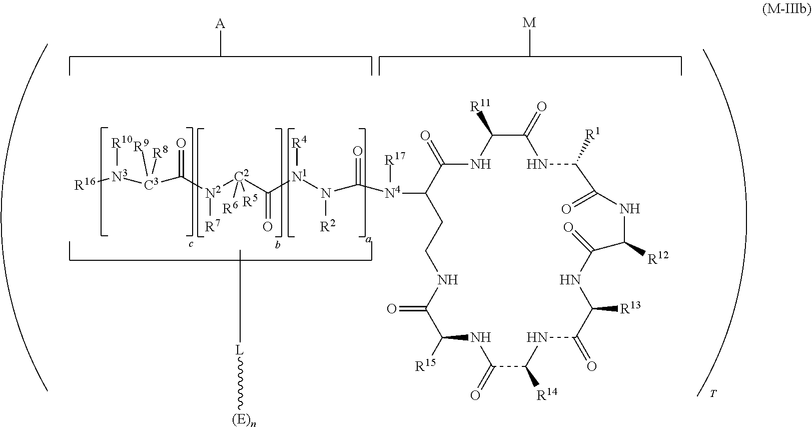

or a pharmaceutically acceptable salt thereof.

In some embodiments, the conjugate is described by formula (M-IIIc):

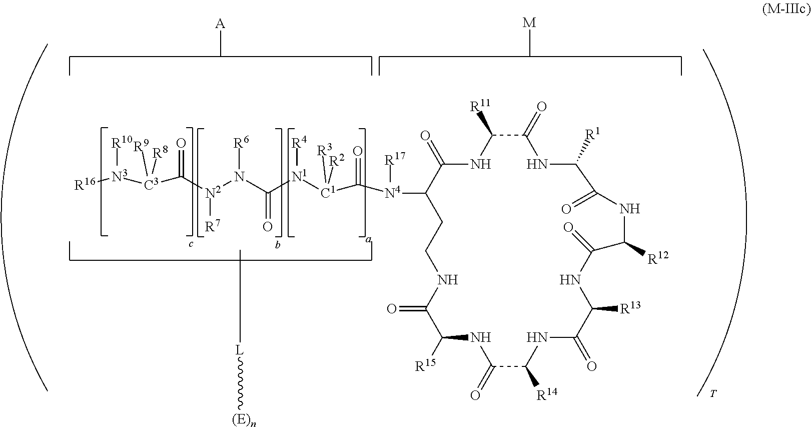

or a pharmaceutically acceptable salt thereof.

In some embodiments, the conjugate is described by formula (M-IIId):

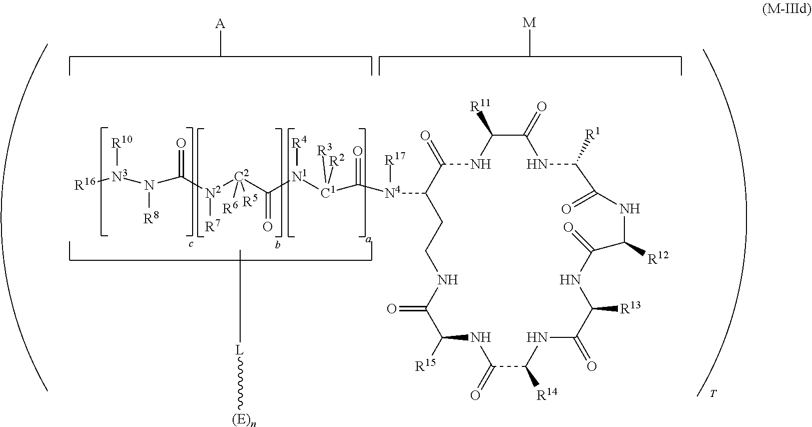

or a pharmaceutically acceptable salt thereof.

In some embodiments, each of R¹ and R¹² is a lipophilic moiety; each of R¹¹, R¹³, and R¹⁴ is, independently, optionally substituted C1-C5 alkamino, a polar moiety, or a positively charged moiety; and each R¹⁵ is a polar moiety; or a pharmaceutically acceptable salt thereof.

In some embodiments, each lipophilic moiety is independently selected from optionally substituted C1-C20 alkyl, optionally substituted C5-C15 aryl, optionally substituted C6-C35 alkaryl, or optionally C5-C10 substituted heteroaryl, or a pharmaceutically acceptable salt thereof.

In some embodiments, each lipophilic moiety is, independently, C1-C8 alkyl, methyl substituted C2-C4 alkyl, (C1-C10)alkylene(C6)aryl, phenyl substituted (C1-C10)alkylene(C6)aryl, or alkyl substituted C4-C9 heteroaryl, or a pharmaceutically acceptable salt thereof.

In some embodiments, each lipophilic moiety is, independently, benzyl, isobutyl, sec-butyl, isopropyl, n-propyl, methyl, biphenylmethyl, n-octyl, or methyl substituted indolyl, or a pharmaceutically acceptable salt thereof.

In some embodiments, each of R¹¹, R¹³, and R¹⁴ is independently optionally substituted C1-C5 alkamino, or a pharmaceutically acceptable salt thereof.

In some embodiments, each of R¹¹, R¹³, and R¹⁴ is CH₂CH₂NH₂, or a pharmaceutically acceptable salt thereof.

In some embodiments, each R¹⁵ is a polar moiety, or a pharmaceutically acceptable salt thereof. In some embodiments, the polar moiety comprises C1-C4 hydroxyalkyl, a carboxylic acid group, an ester group, or an amide group, or a pharmaceutically acceptable salt thereof. In some embodiments, the polar moiety is C1-C4 hydroxyalkyl, or a pharmaceutically acceptable salt thereof. In some embodiments, the polar moiety is CHCH₃OH, or a pharmaceutically acceptable salt thereof.

In some embodiments, R¹⁷ is H. In some embodiments, R¹⁷ is methyl.

In some embodiments of this aspect, the conjugate is described by formula (M-IVa):

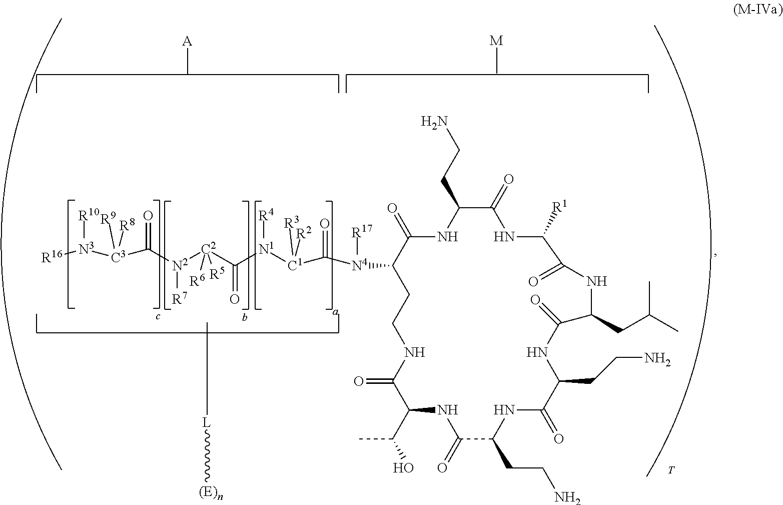

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In some embodiments, the conjugate is described by formula (M-IVa-1):

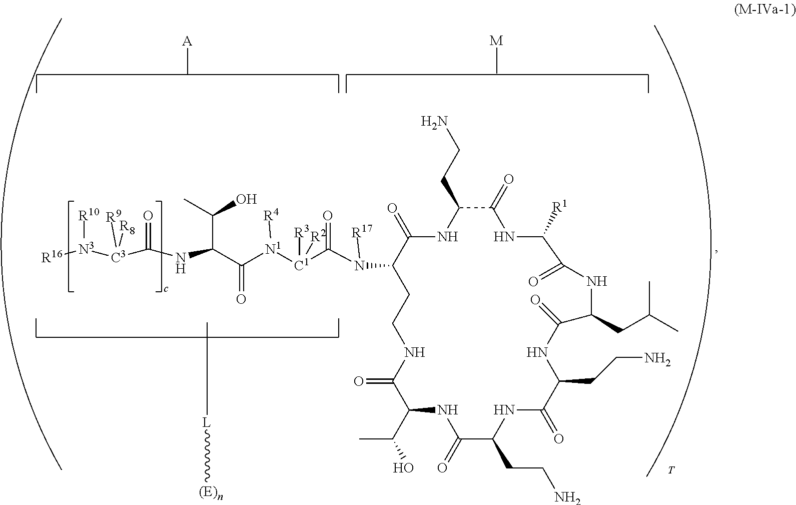

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In some embodiments, the conjugate is described by formula (M-IVa-2):

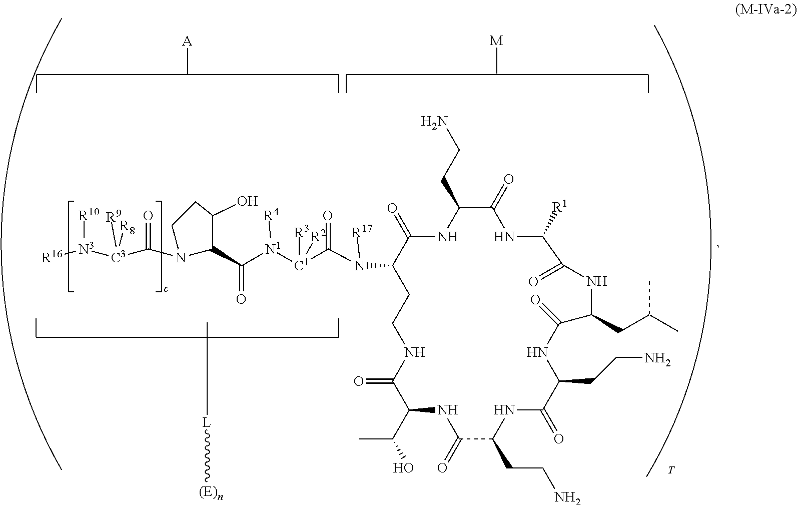

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In some embodiments, the conjugate is described by formula (M-IVa-3):

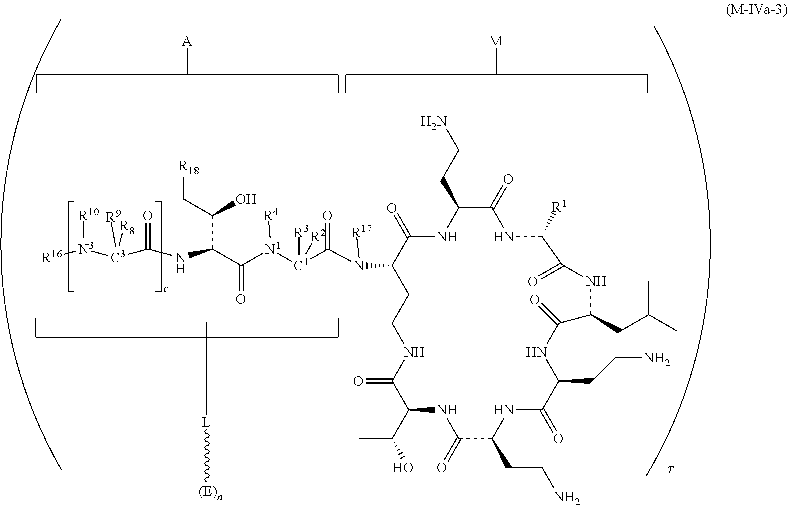

wherein each R¹ is optionally substituted benzyl, CH₂C(C₃)₂, cyclopropylmethyl, cyclopentylmethyl, cyclohexylmethyl, naphthylmethyl, optionally substituted thiophenylmethyl, or optionally substituted furanylmethyl; and R¹⁸ is a lipophilic moiety, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; or a pharmaceutically acceptable salt thereof.

In some embodiments, R¹⁸ is C1-C6 alkyl or benzyl.

In some embodiments, the conjugate is described by formula (M-IVb):

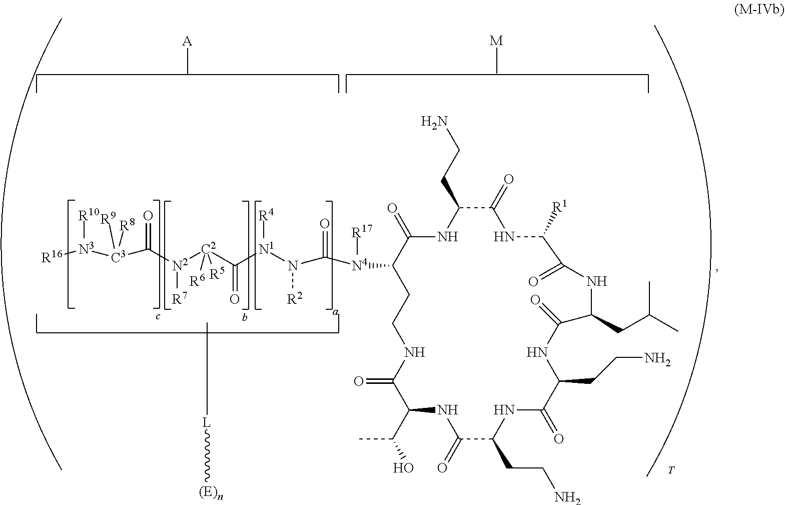

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In so embodiments the conjugate is described by formula (M-IVb-1):

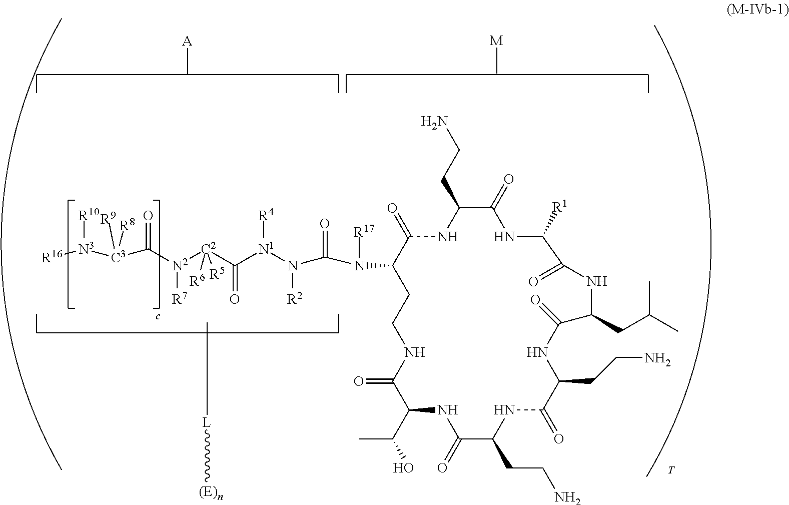

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In some embodiments, R² is H or C1-C8 alkyl.

In some embodiments, the conjugate is described by formula (M-IVc):

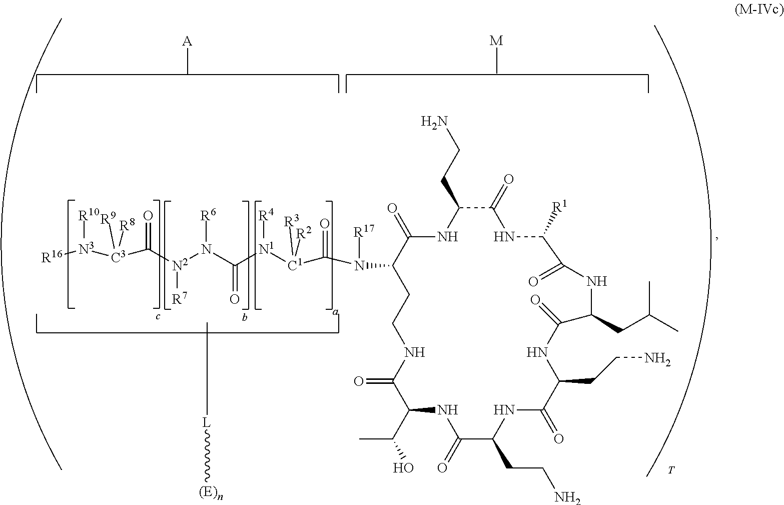

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In some embodiments, the conjugate is described by formula (M-IVc-1):

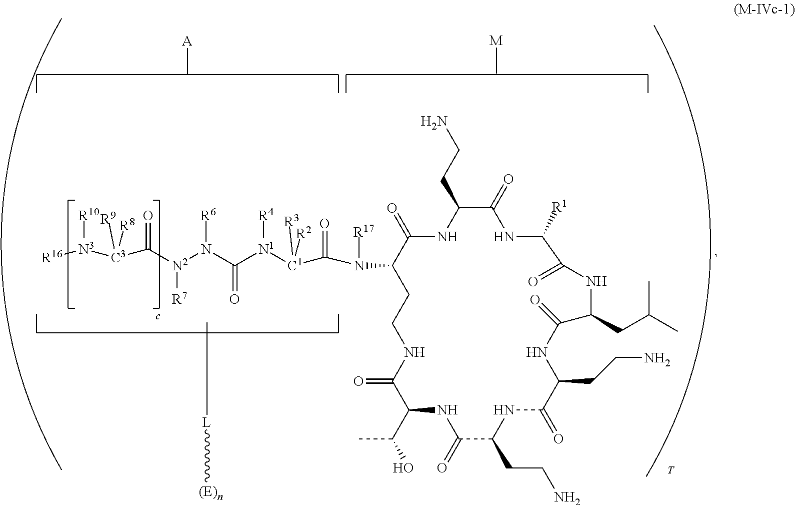

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In some embodiments, R⁶ is H or C1-C8 alkyl.

In some embodiments, the conjugate is described by formula (M-IVd):

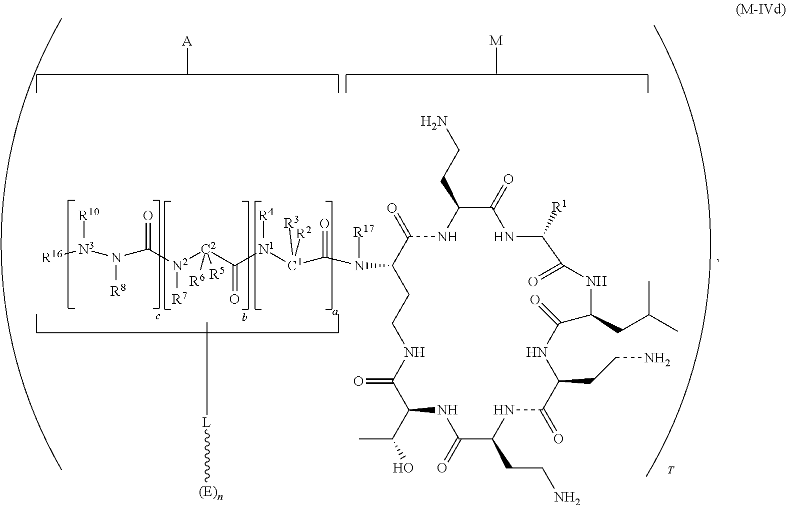

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl, or a pharmaceutically acceptable salt thereof.

In some embodiments, R⁸ is H or C1-C8 alkyl.

In some embodiments, R¹⁷ is H. In some embodiments, R¹⁷ is methyl.

In some embodiments, the conjugate is described by formula (M-V):

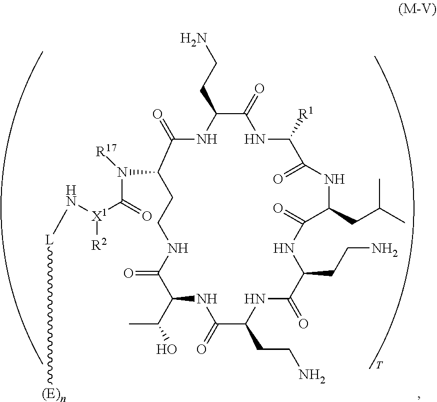

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethy; and each R² is H, a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; or a pharmaceutically acceptable salt thereof.

In some embodiments, the conjugate is described by formula (M-Va):

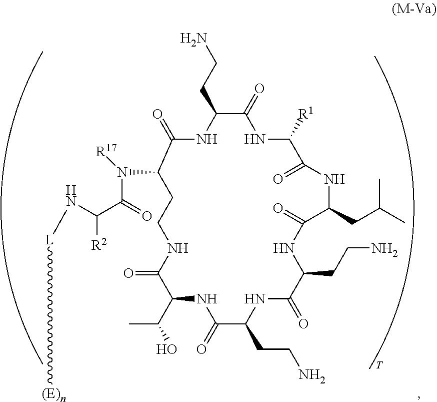

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; and each R² is H, a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; or a pharmaceutically acceptable salt thereof.

In some embodiments, the conjugate is described by formula (M-Vb):

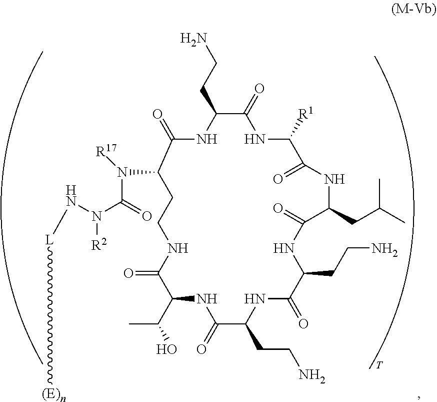

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; and each R² is H, a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; or a pharmaceutically acceptable salt thereof.

In some embodiments, R² is H or C1-C8 alkyl.

In some embodiments, the conjugate is described by formula (M-V-1):

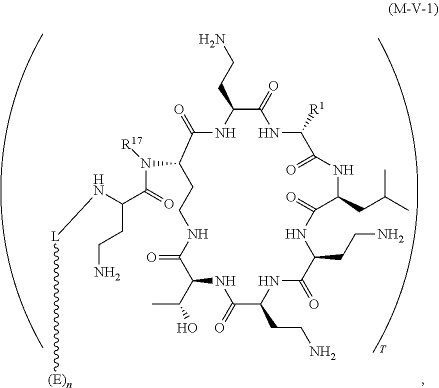

or a pharmaceutically acceptable salt thereof.

In some embodiments, the conjugate is described by formula (M-VI):

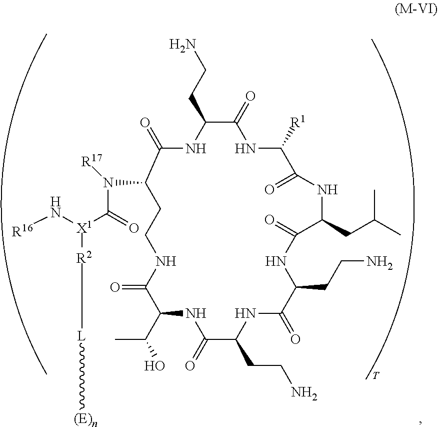

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each R² is optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene; and each R^{IG} is H, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; or a pharmaceutically acceptable salt thereof.

In some embodiments, the conjugate is described by formula (M-VIa):

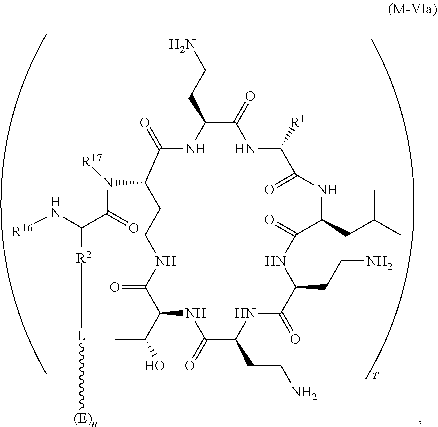

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (M-VIb):

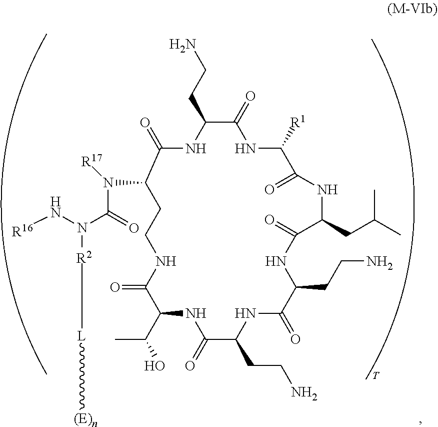

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (M-VI-1):

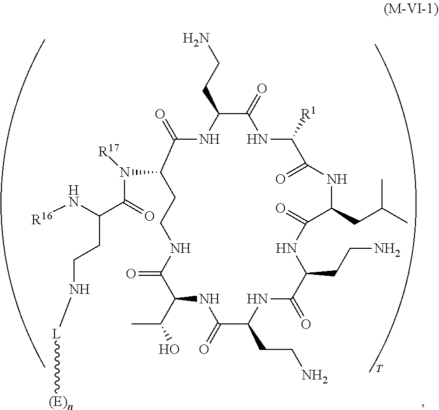

wherein each R¹⁶ is H, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; or a pharmaceutically acceptable salt thereof.

In some embodiments of this aspect, conjugate is described by formula (M-VII):

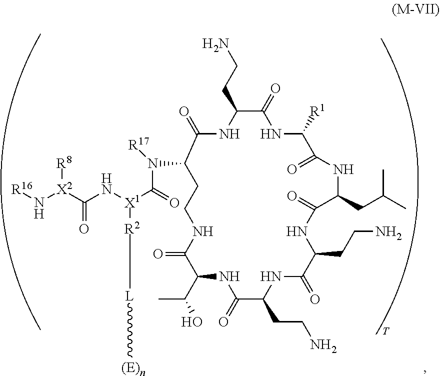

wherein each R1 is optionally substituted benzyl, CH2CH(CH3)2, cyclopropylmethyl, cyclopentylmethyl, cyclohexylmethyl, naphthylmethyl, optionally substituted thiophenylmethyl, or optionally substituted furanylmethyl; each R² is optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene; each Ra is a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; and each R¹⁶ is H, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; or a pharmaceutically acceptable salt thereof.

In some embodiments, the conjugate is described by formula (M-VIIa):

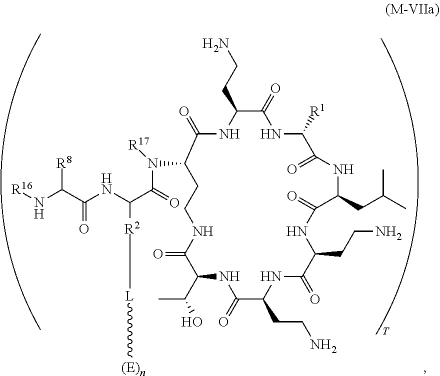

wherein each R1 is optionally substituted benzyl, CH2CH(CH3)2, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (M-VIIb):

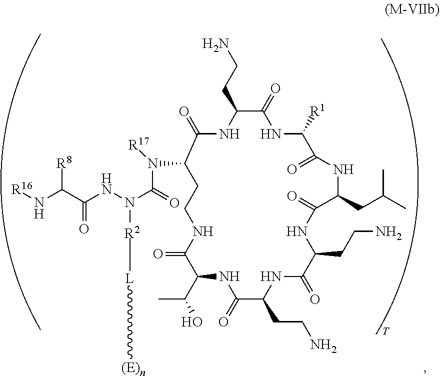

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (M-VIIc):

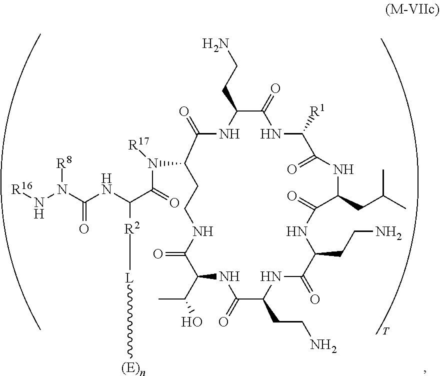

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (M-VII-1):

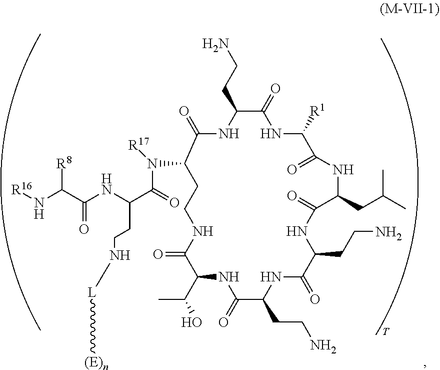

or a pharmaceutically acceptable salt thereof.

In some embodiments, the conjugate is described by formula (M-VII-2):

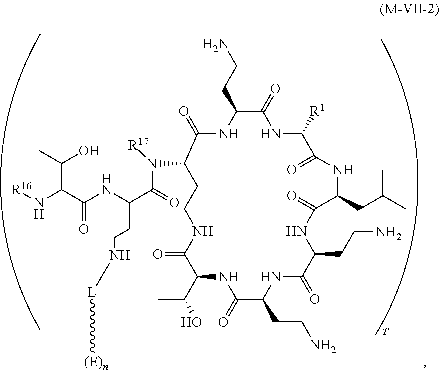

wherein each R¹⁶ is H, optionally substituted C1-C20 alkyl, or optionally substituted C1-C20 heteroalkyl, or a pharmaceutically acceptable salt thereof.

In some embodiments, the conjugate is described by formula (M-VII-3):

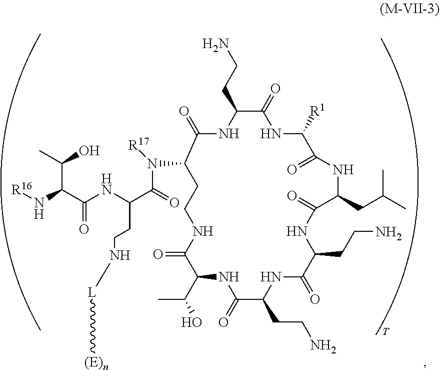

or a pharmaceutically acceptable salt thereof.

In some embodiments, the conjugate is described by formula (M-VII-4):

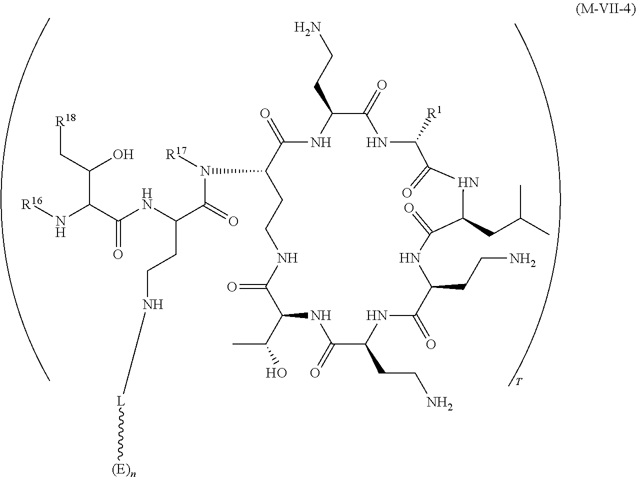

wherein each R¹⁶ is H, optionally substituted C1-C20 alkyl, or optionally substituted C1-C20 heteroalkyl; and R¹⁸ is a lipophilic moiety, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; or a pharmaceutically acceptable salt thereof.

In some embodiments, the conjugate is described by formula (M-VII-5):

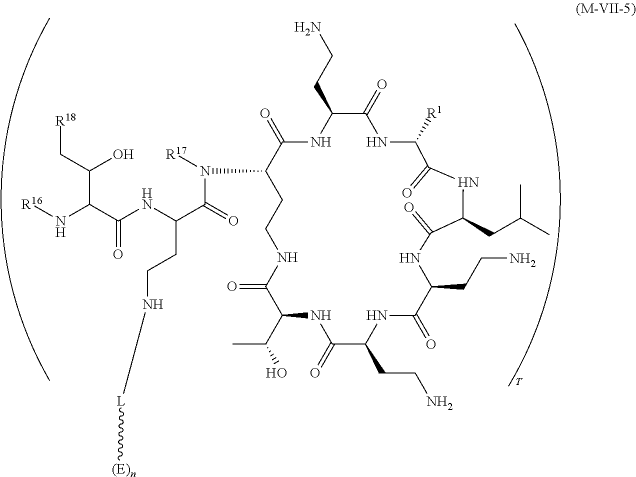

or a pharmaceutically acceptable salt thereof.

In some embodiments of this aspect, the conjugate is described by formula (M-VIII):

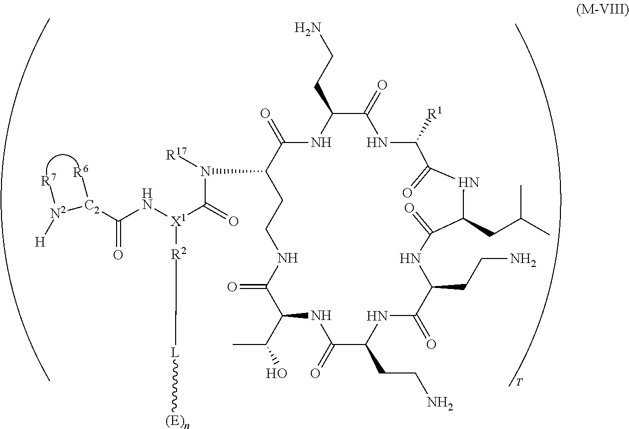

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; and each R² is optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene; each R⁶, R⁷, N², and C² together form an optionally substituted 5-8 membered ring comprising optionally substituted C3-C7 heterocycloalkyl comprising a nitrogen atom and additional 0-2 heteroatoms independently selected from N, O, and S, or optionally substituted C2-C7 heteroaryl comprising a nitrogen atom and additional 0-2 heteroatoms independently selected from N, O, and S; or a pharmaceutically acceptable salt thereof.

In some embodiments, the conjugate is described by formula (M-VIIIa):

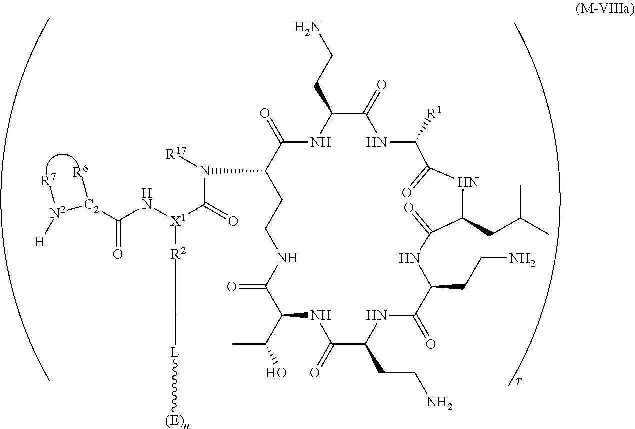

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (M-VIIIb):

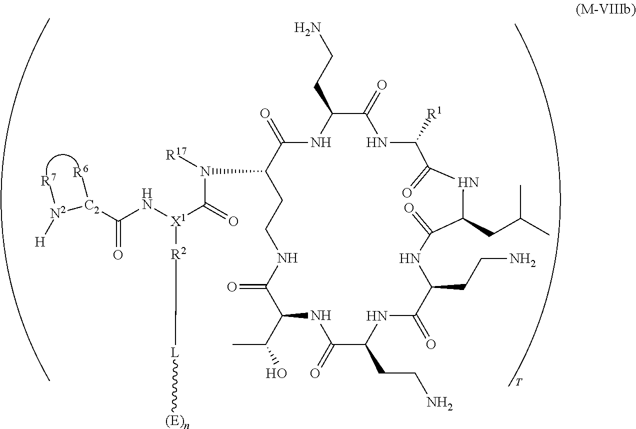

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (M-VIII-1):

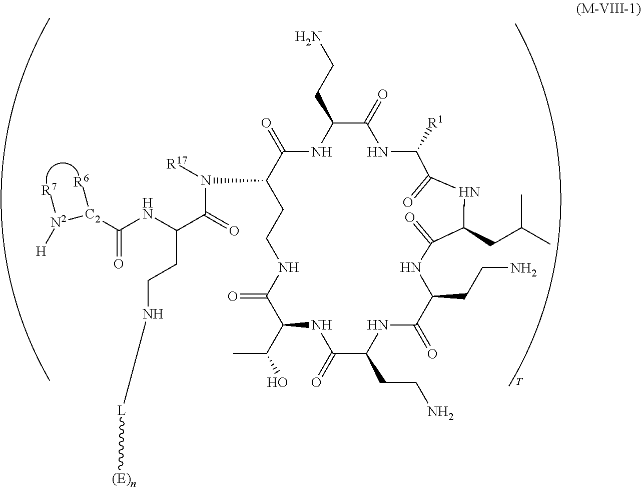

or a pharmaceutically acceptable salt thereof.

In some embodiments, the conjugate is described by formula (M-VIII-2):

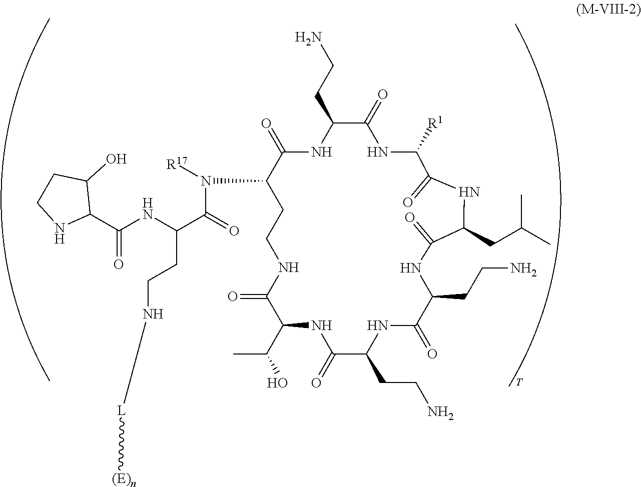

or a pharmaceutically acceptable salt thereof.

In some embodiments, the conjugate is described by formula (M-VIII-3):

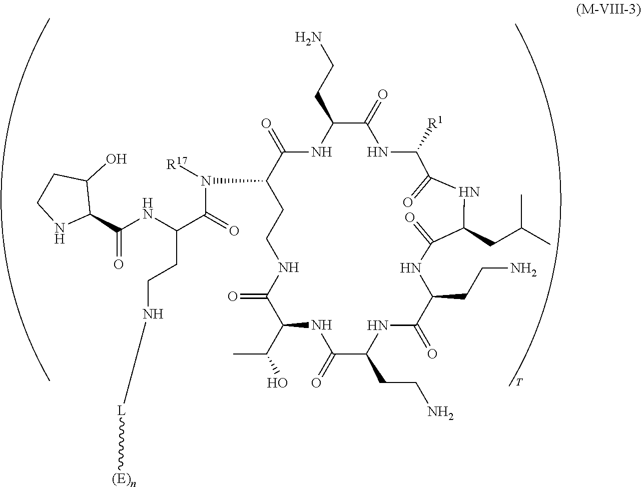

or a pharmaceutically acceptable salt thereof.

In some embodiments, the conjugate is described by formula (M-VIII-4):

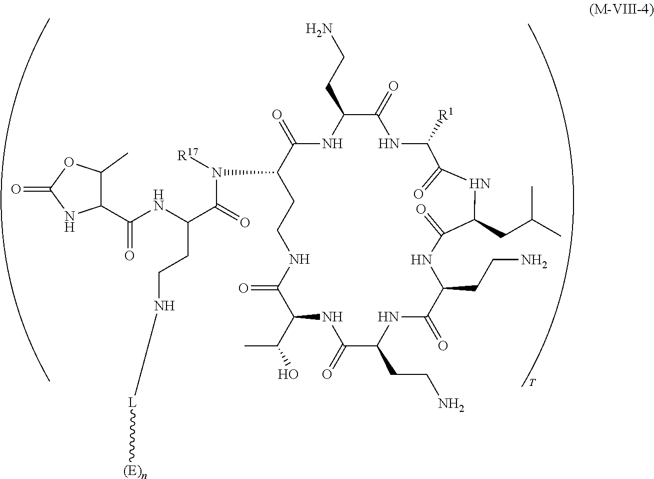

or a pharmaceutically acceptable salt thereof.

In some embodiments, the conjugate is described by formula (M-VIII-5):

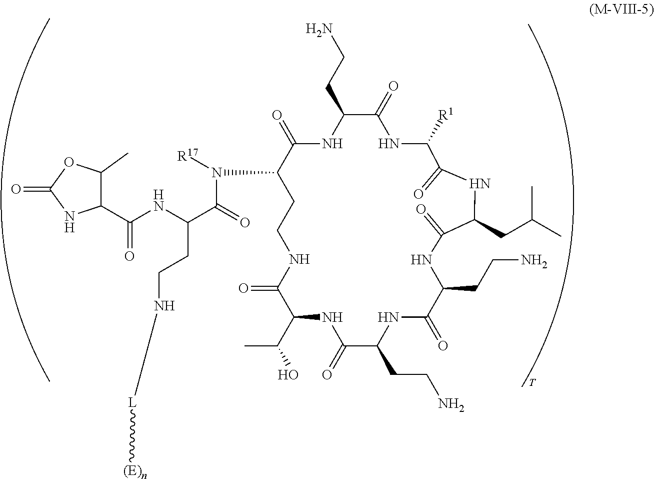

or a pharmaceutically acceptable salt thereof.

In some embodiments, the conjugate is described by formula (M-IX):

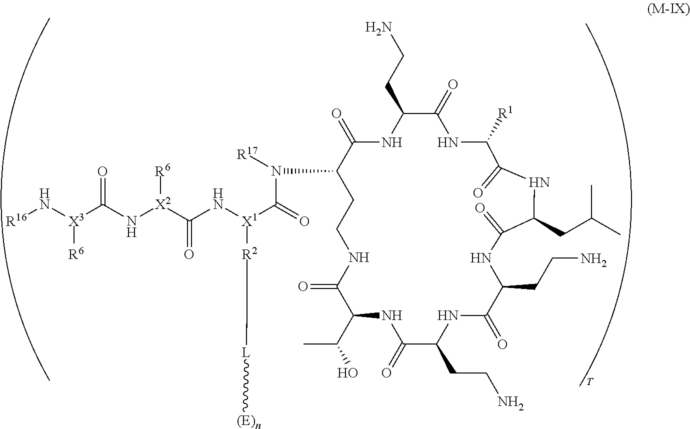

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each R² is optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene; each of R⁶ and R⁸ is, independently, a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; and each R^{t}e is H, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; or a pharmaceutically acceptable salt thereof.

In some embodiments, the conjugate is described by formula (M-IXa):

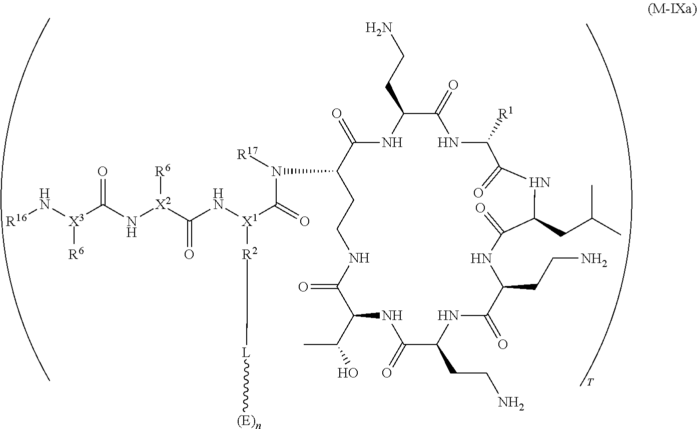

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (M-IXb):

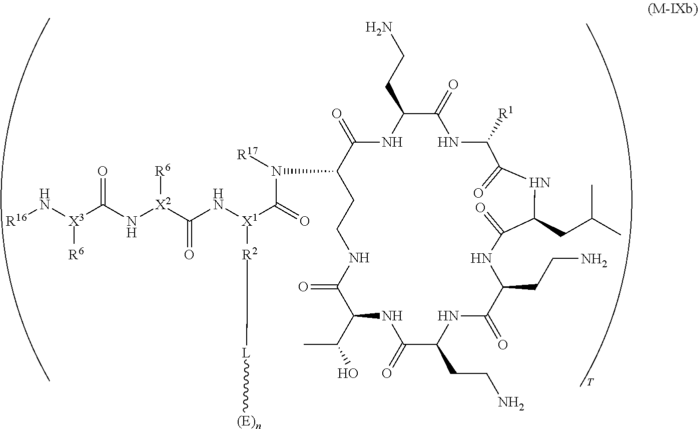

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (M-IXc):

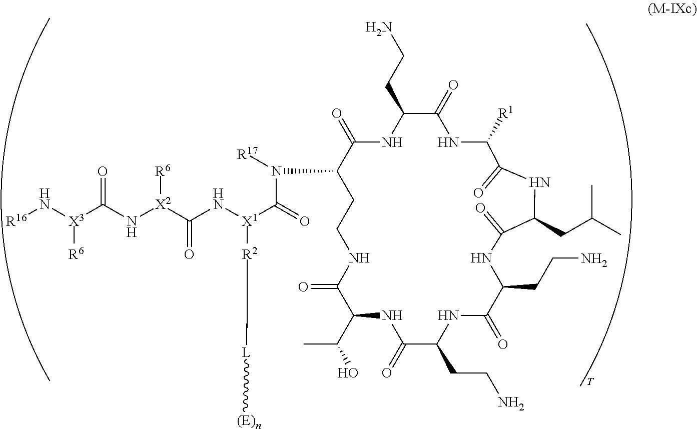

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (M-IXd):

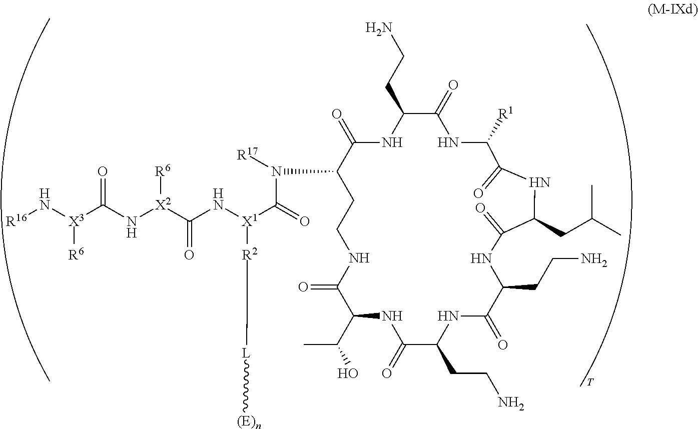

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (M-IX-1):

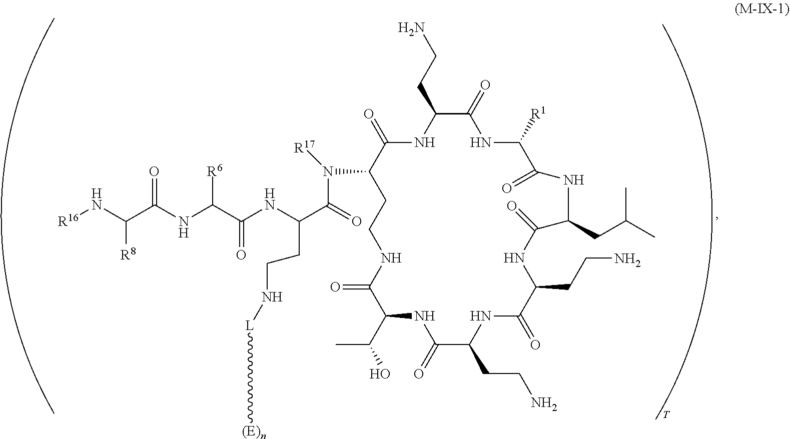

or a pharmaceutically acceptable salt thereof.

In some embodiments, the conjugate is described b formula (M-IX-2):

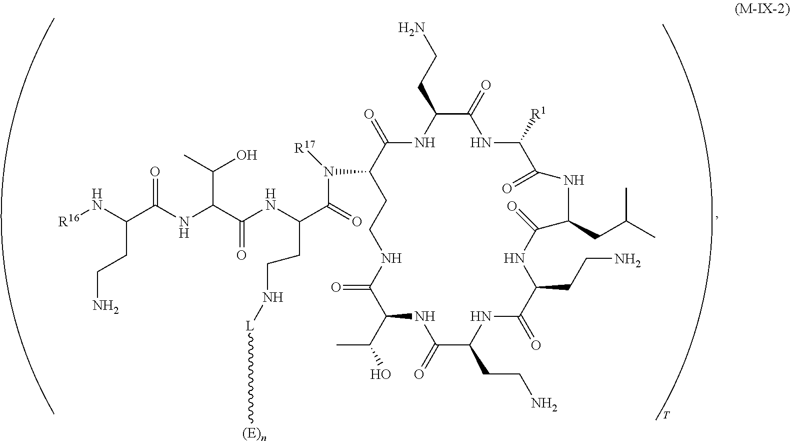

wherein each R¹⁶ is H, optionally substituted C1-C20 alkyl, or optionally substituted C1-C20 heteroalkyl; or a pharmaceutically acceptable salt thereof.

In some embodiments the conjugate is described by formula (M-IX-2a):

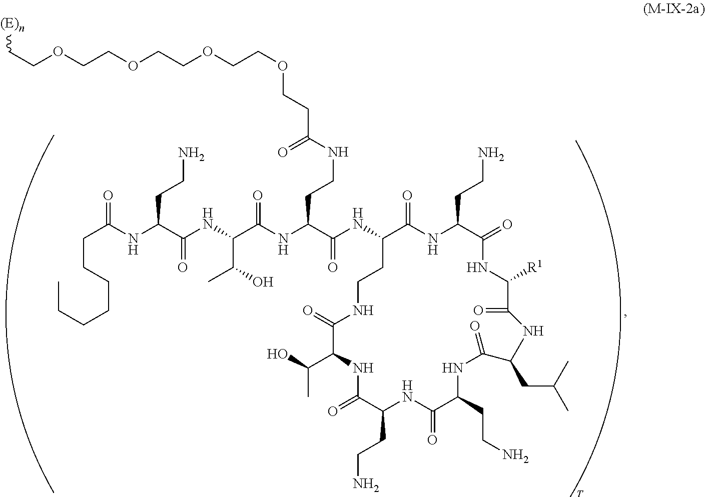

or a pharmaceutically acceptable salt thereof.

In some embodiments, the conjugate is described by formula (M-IX-3):

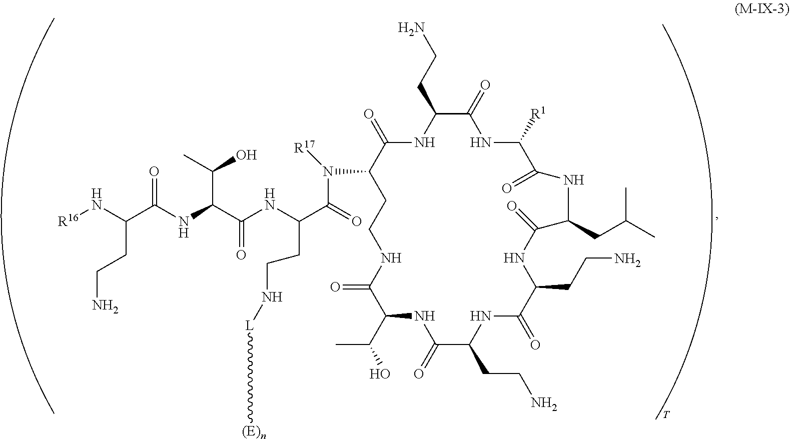

wherein each R¹⁶ is H, optionally substituted C1-C20 alkyl, or optionally substituted C1-C20 heteroalkyl; or a pharmaceutically acceptable salt thereof.

In some embodiments, the conjugate is described by formula (M-IX-4):

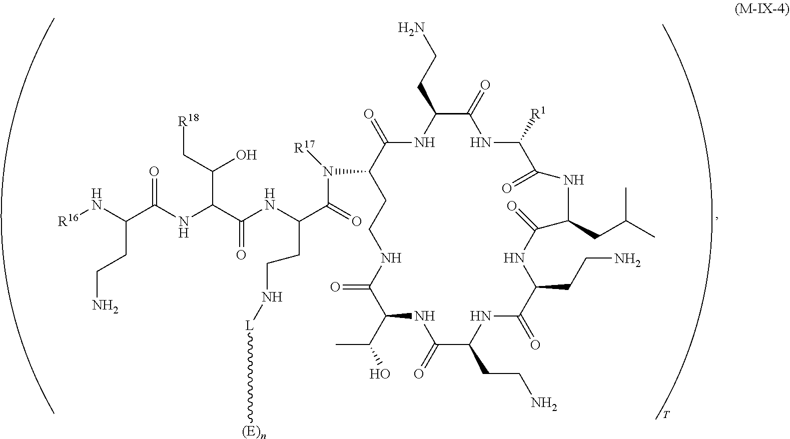

wherein each R¹⁶ is H, optionally substituted C1-C20 alkyl, or optionally substituted C1-C20 heteroalkyl; and R¹⁸ is a lipophilic moiety, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; or a pharmaceutically acceptable salt thereof.

In some embodiments, the conjugate is described b formula (M-IX-5):

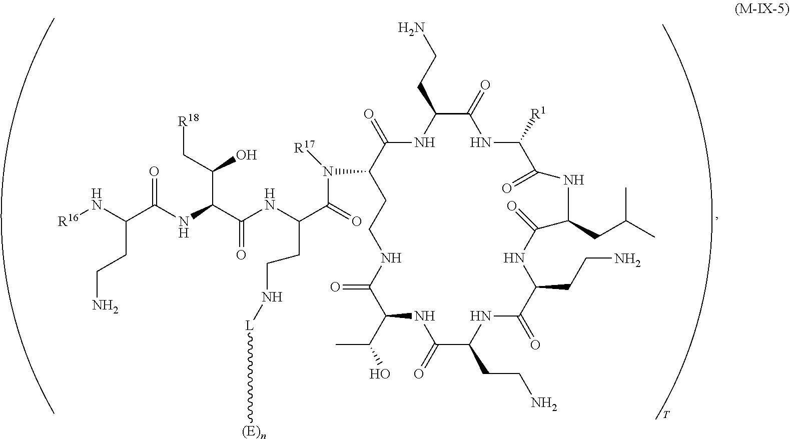

or a pharmaceutically acceptable salt thereof.

In some embodiments of this aspect, the conjugate is described by formula (M-X):

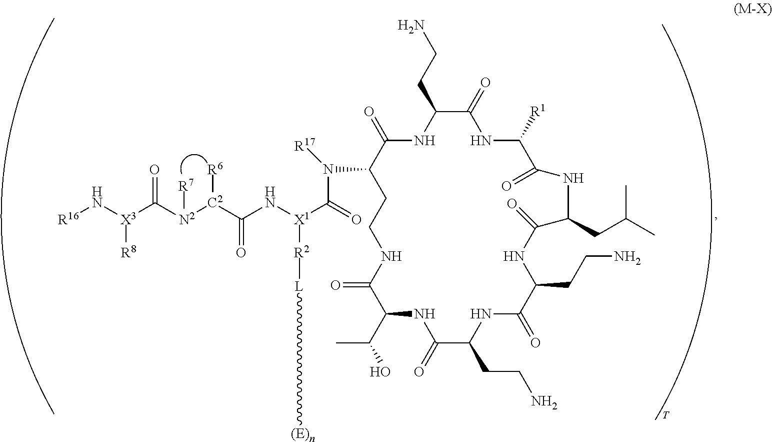

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each R² is optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene; each R⁶, R⁷, N², and C² together form an optionally substituted 5-8 membered ring comprising optionally substituted C3-C7 heterocycloalkyl comprising a nitrogen atom and additional 0-2 heteroatoms independently selected from N, O, and S, or optionally substituted C2-C7 heteroaryl comprising a nitrogen atom and additional 0-2 heteroatoms independently selected from N, O, and S; each R⁶ is a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; each R¹⁶ is H, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments, the conjugate is described by formula (M-Xa):

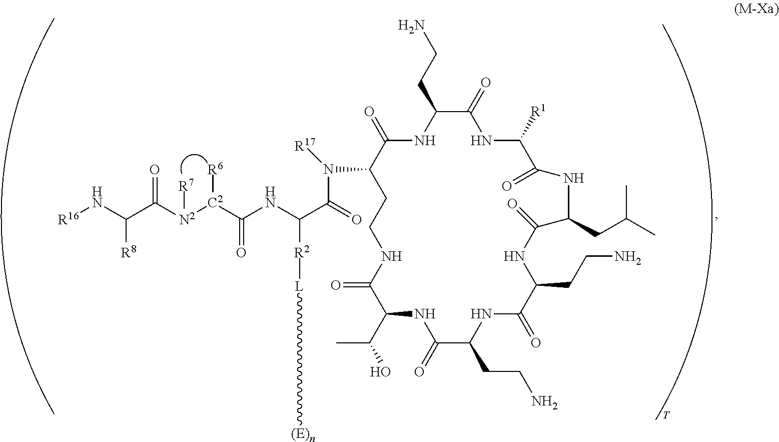

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (M-Xb):

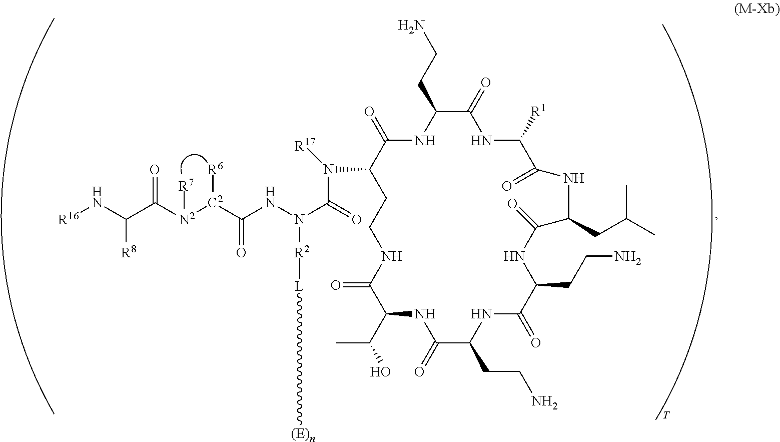

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments, the conjugate is described by formula (M-Xc):

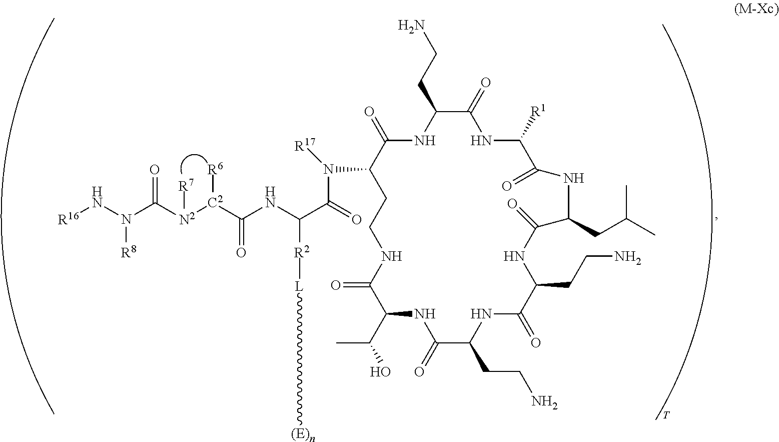

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; or a pharmaceutically acceptable salt thereof.

In some embodiments the conjugate is described by formula (M-X-1):

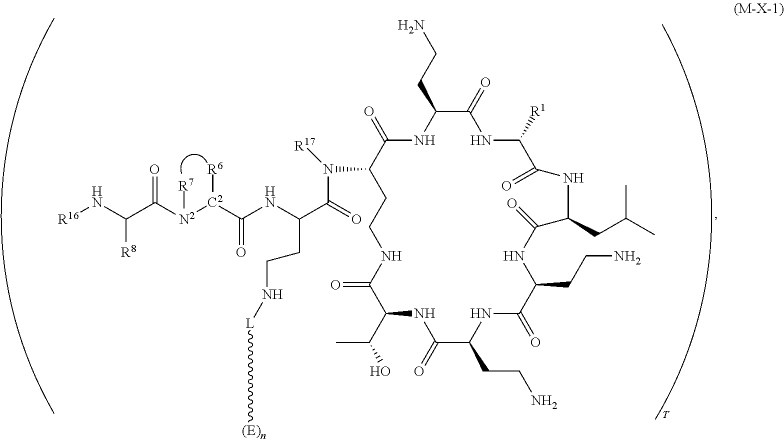

or a pharmaceutically acceptable salt thereof.

In some embodiments, the conjugate is described by formula (M-XI):

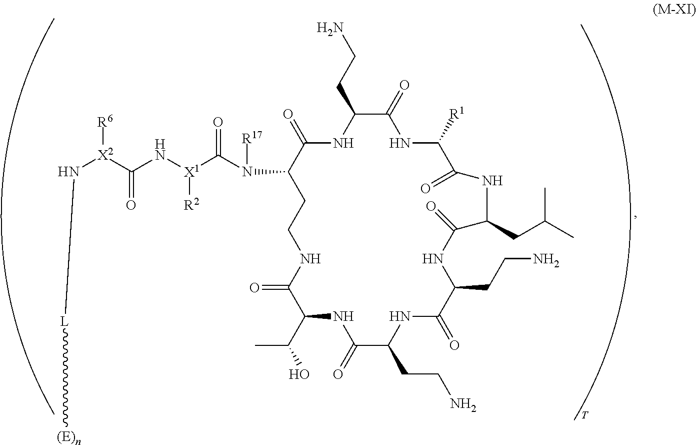

wherein each R¹ is optionally substituted benzyl. CH₂CH(CH₃)₂, cyclopropylmethyl, cyclopentylmethyl, cyclohexylmethyl, naphthylmethyl, optionally substituted thiophenylmethyl, or optionally substituted furanylmethyl; and each of R² and R⁶ is, independently, a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments, the conjugate is described by formula (M-XIa):

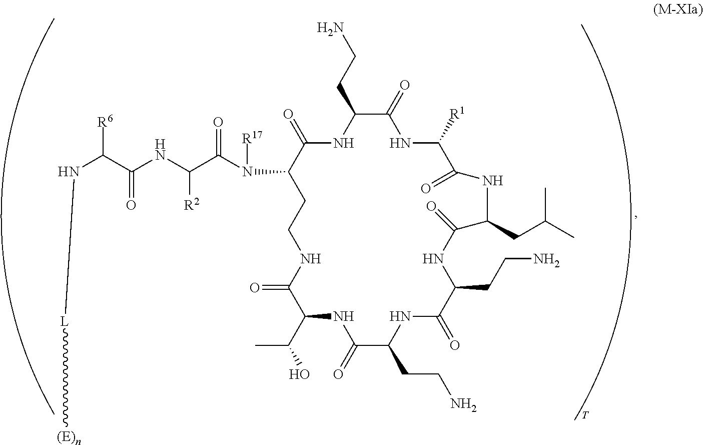

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; and or a pharmaceutically acceptable salt thereof.

In some embodiments, the conjugate is described by formula (M-XIb):

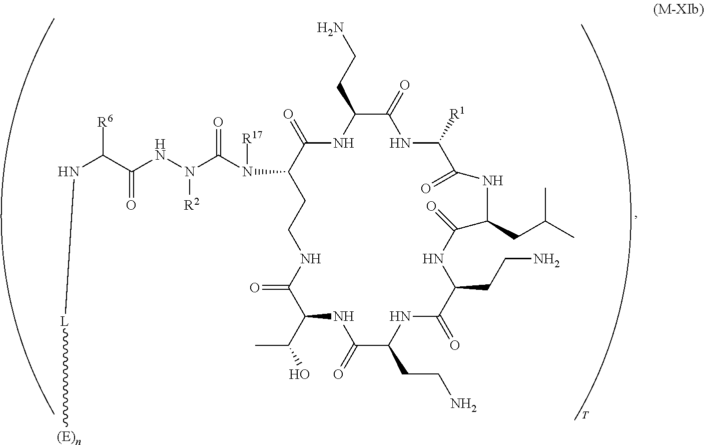

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; and or a pharmaceutically acceptable salt thereof.

In some embodiments, the conjugate is described by formula (M-XIc):

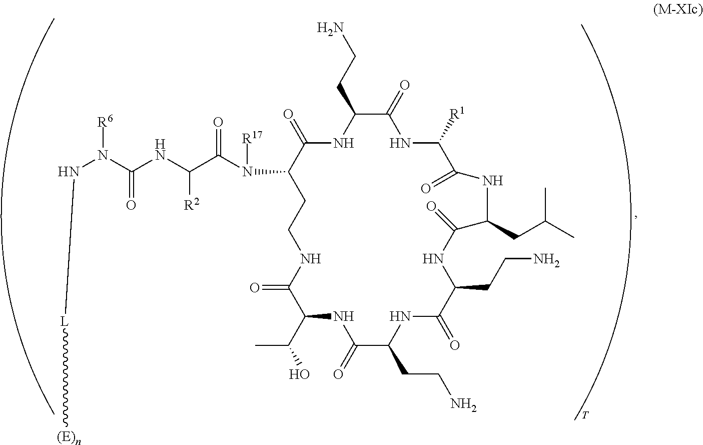

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; and or a pharmaceutically acceptable salt thereof.

In some embodiments, the conjugate is described by formula (M-XI-1):

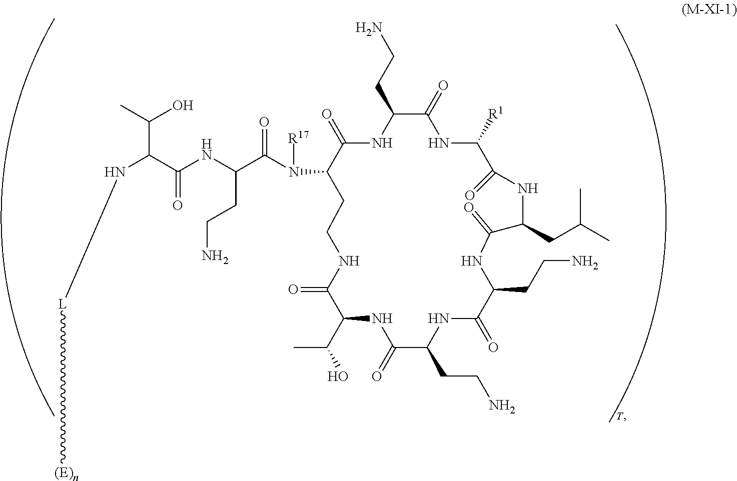

or a pharmaceutically acceptable salt thereof.

In some embodiments, the conjugate is described by formula (M-XI-2):

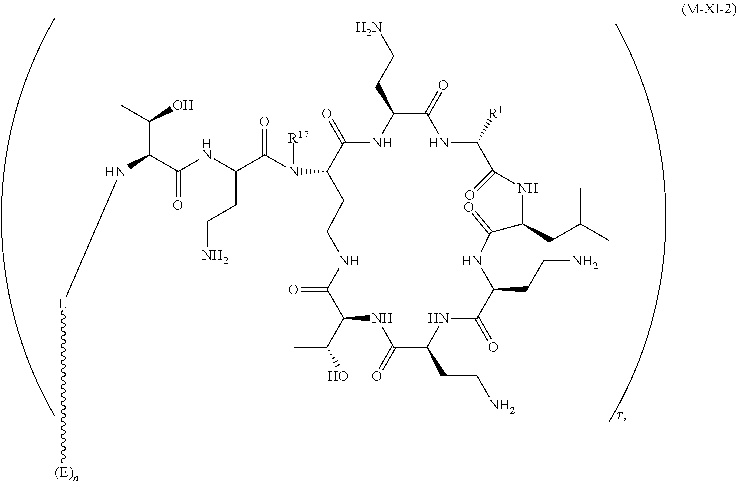

or a pharmaceutically acceptable salt thereof.

In some embodiments, the conjugate is described by formula (M-XI-3):

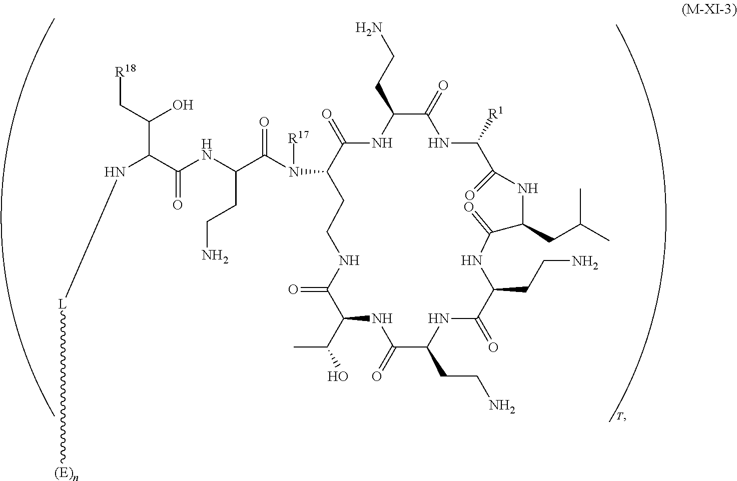

wherein R¹⁸ is a lipophilic moiety, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; or a pharmaceutically acceptable salt thereof.

In some embodiments, the conjugate is described by formula (M-XI-4):

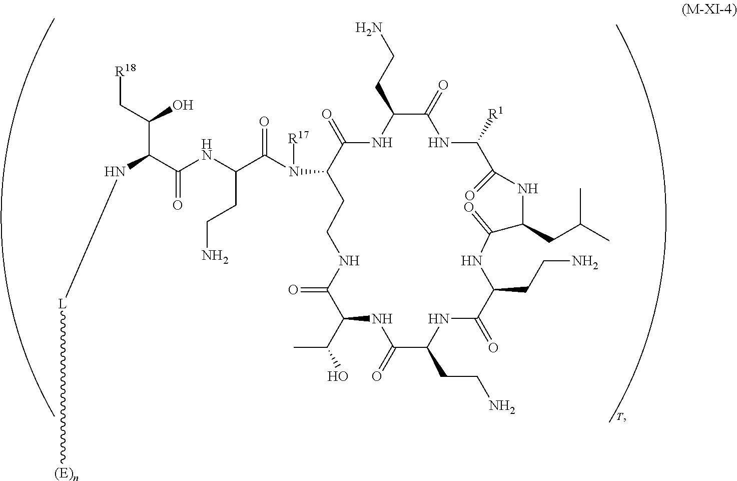

or a pharmaceutically acceptable salt thereof.

In some embodiments, the conjugate is described by formula (M-XI-5):

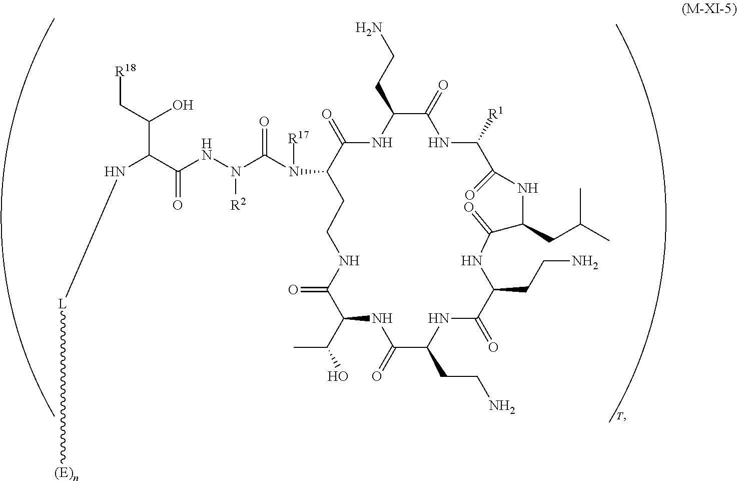

wherein R¹⁸ is a lipophilic moiety, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; and R² is H or C1-C8 alkyl; or a pharmaceutically acceptable salt thereof.

In some embodiments, the conjugate is described by formula (M-XI-6):

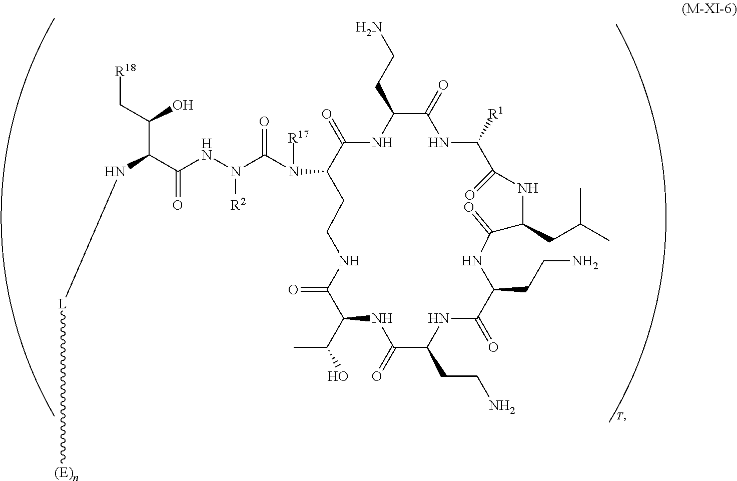

or a pharmaceutically acceptable salt thereof.

In some embodiments, the conjugate is described by formula (M-XII):

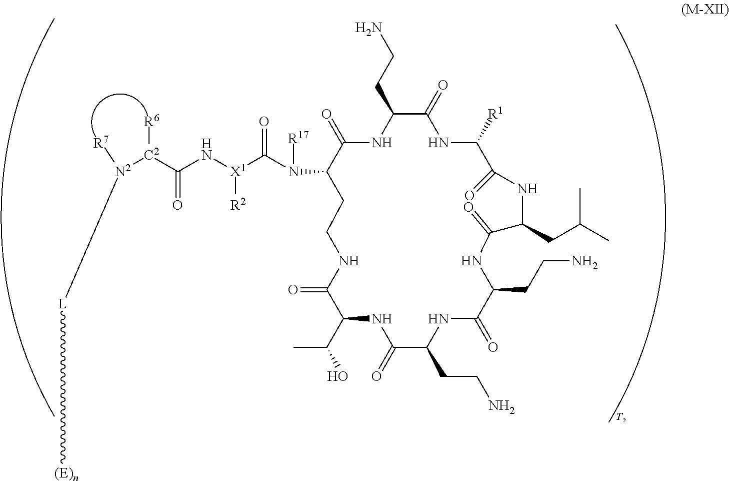

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each R² is a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; and each R⁶, R⁷, N², and C² together form an optionally substituted 5-8 membered ring comprising optionally substituted C3-C7 heterocycloalkyl comprising a nitrogen atom and additional 0-2 heteroatoms independently selected from N, O, and S, or optionally substituted C2-C7 heteroaryl comprising a nitrogen atom and additional 0-2 heteroatoms independently selected from N, O, and S; or a pharmaceutically acceptable salt thereof.

In some embodiments, the conjugate is described by formula (M-XIIa):

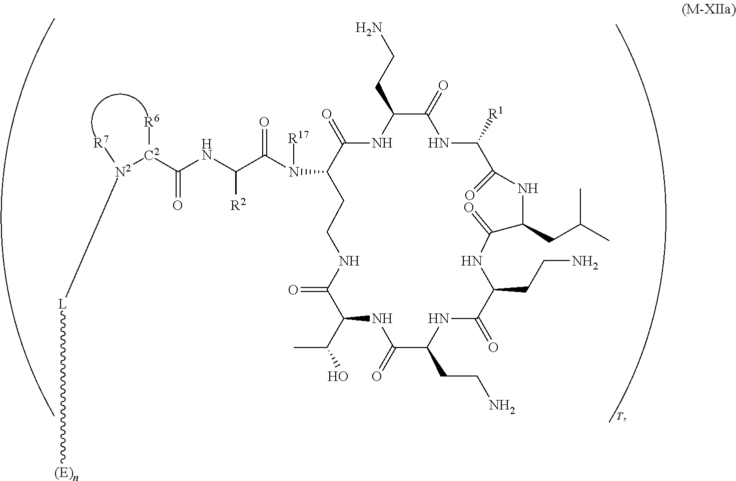

or a pharmaceutically acceptable salt thereof.

In some embodiments, the conjugate is described by formula (M-XIIb):

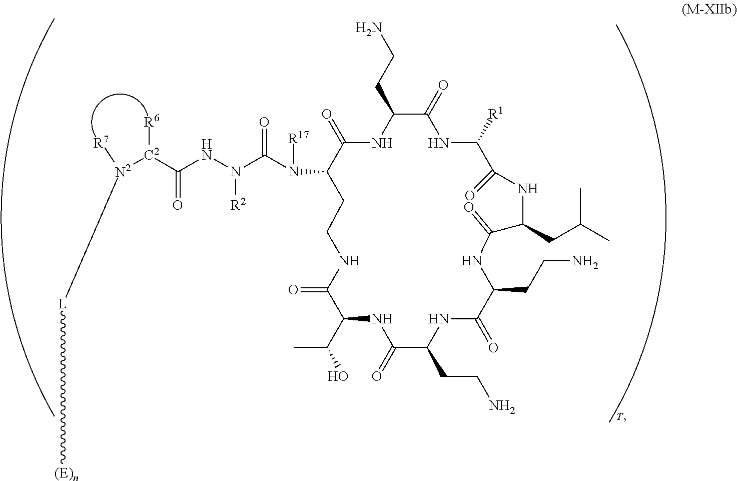

or a pharmaceutically acceptable salt thereof.

In some embodiments of this aspect, the conjugate is described by formula (M-XIII):

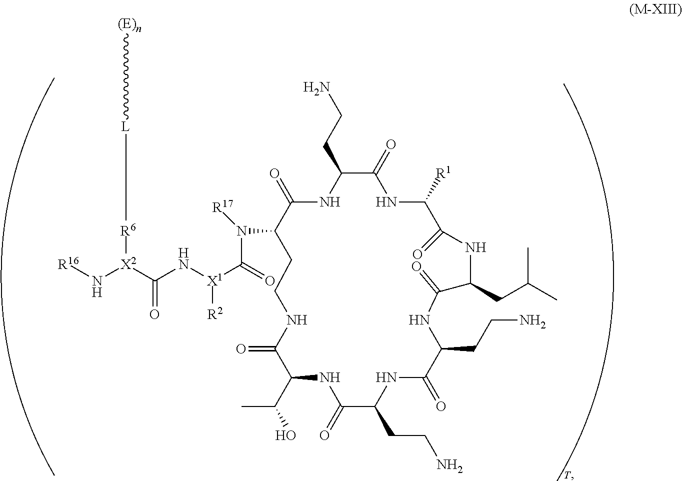

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each R² is a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; each R^{a} is optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene; and each R^{IG} is H, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; or a pharmaceutically acceptable salt thereof.

In some embodiments, the conjugate is described by formula (M-XIIIa):

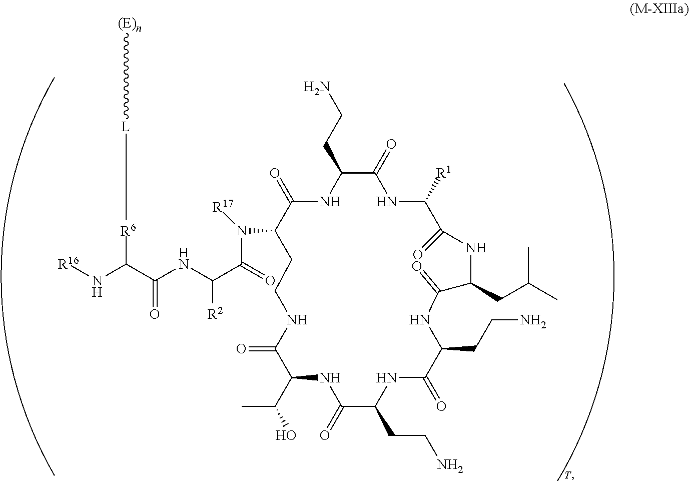

or a pharmaceutically acceptable salt thereof.

In some embodiments, the conjugate is described by formula (M-XIIIb):

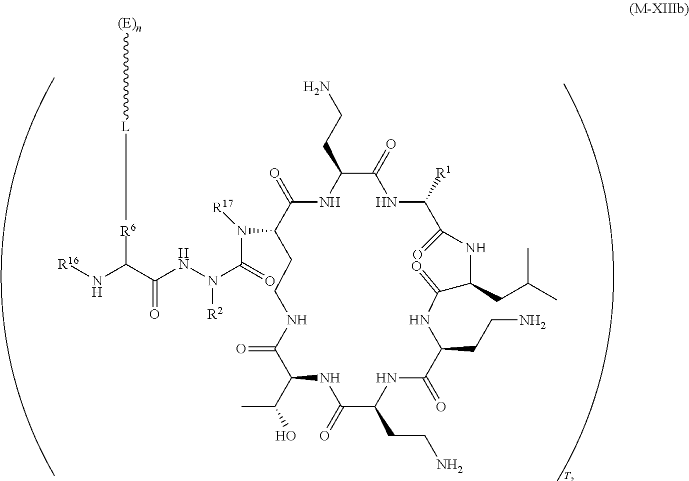

or a pharmaceutically acceptable salt thereof.

In some embodiments, the conjugate is described by formula (M-XIIIc):

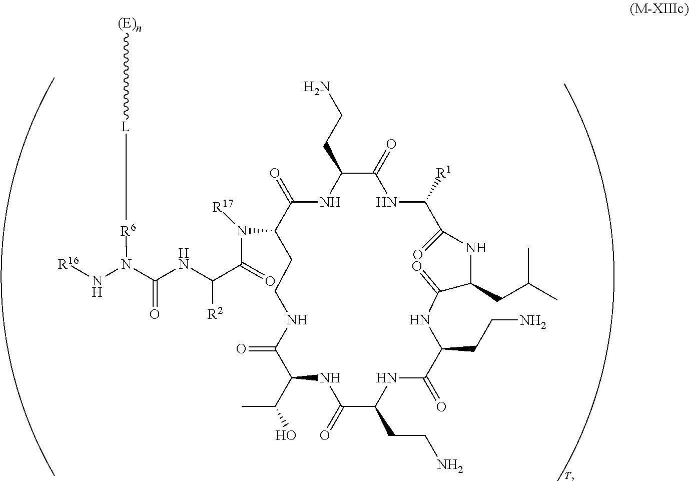

or a pharmaceutically acceptable salt thereof.

In some embodiments, the conjugate is described by formula (M-XIII-1):

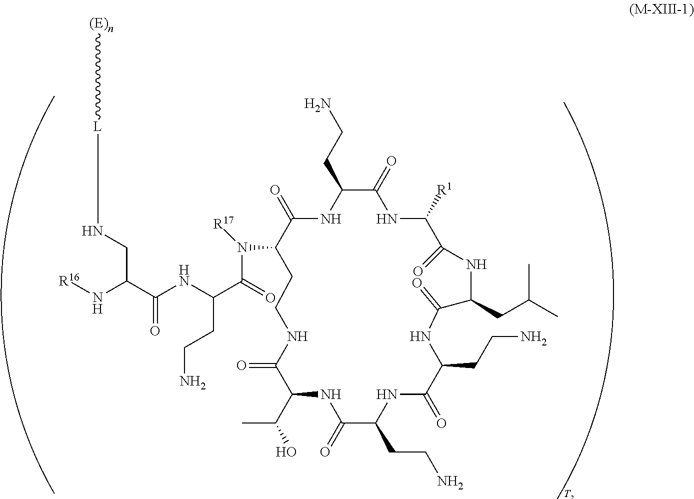

wherein each R¹⁶ is H or optionally substituted C6-C35 heteroalkaryl; or a pharmaceutically acceptable salt thereof.

In some embodiments, the conjugate is described by formula (M-XIII-2):

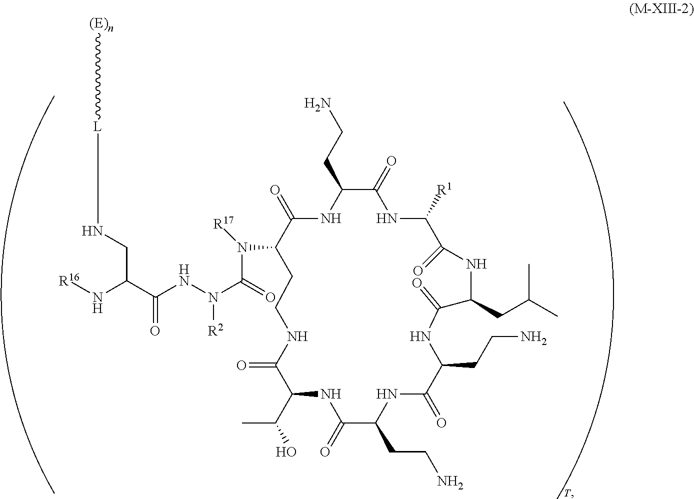

wherein each R¹⁶ is H or optionally substituted C6-C35 heteroalkaryl; and R² is H or C1-C8 alkyl; or a pharmaceutically acceptable salt thereof.

In some embodiments of this aspect, the conjugate is described b formula (M-XIV):

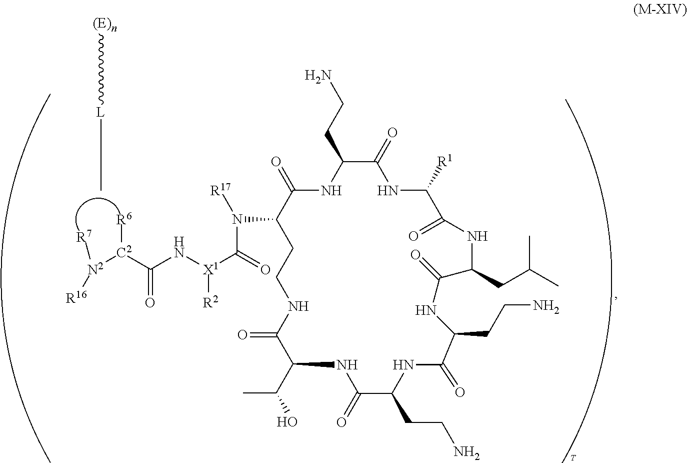

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each R² is a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; each R⁶, R⁷, N², and C² together form an optionally substituted 5-8 membered ring comprising optionally substituted C3-C7 heterocycloalkyl comprising a nitrogen atom and additional 0-2 heteroatoms independently selected from N, O, and S. or optionally substituted C2-C7 heteroaryl comprising a nitrogen atom and additional 0-2 heteroatoms independently selected from N, O, and S; and each R¹⁶ is H, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; or a pharmaceutically acceptable salt thereof.

In some embodiments, the conjugate is described by formula (M-XIVa):

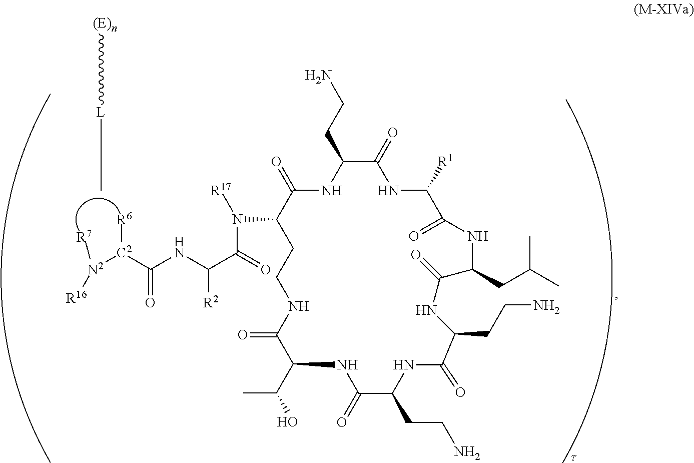

or a pharmaceutically acceptable salt thereof.

In some embodiments, the conjugate is described by formula (M-XIVb):

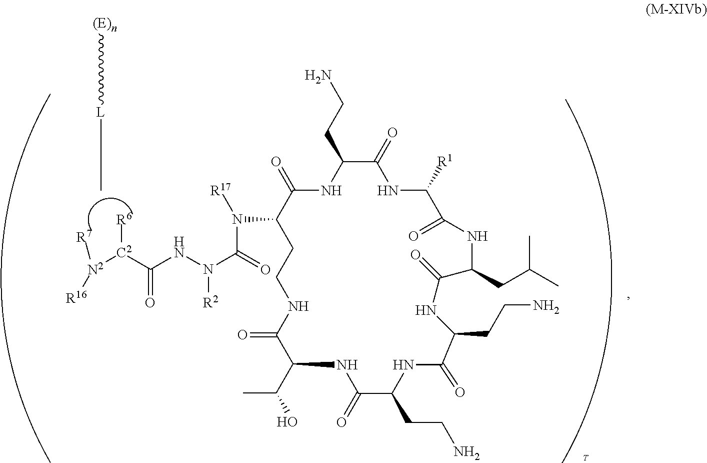

or a pharmaceutically acceptable salt thereof.

In some embodiments, the conjugate is described by formula (M-XIV-1):

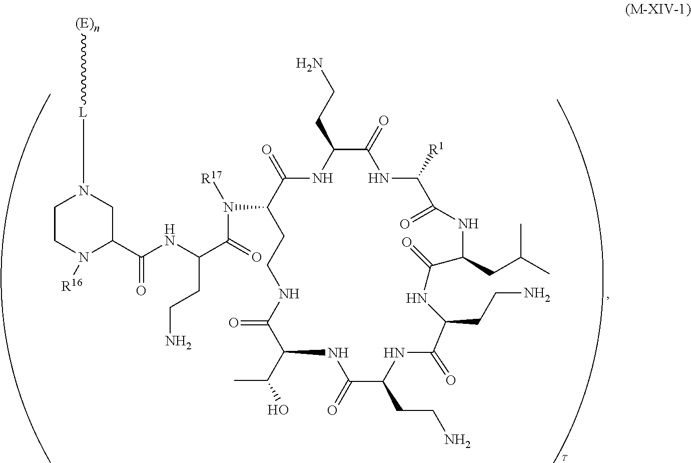

wherein each R¹⁶ is H or optionally substituted C6-C35 heteroalkaryl; or a pharmaceutically acceptable salt thereof.

In some embodiments of this aspect, the conjugate is described by formula (M-XV):

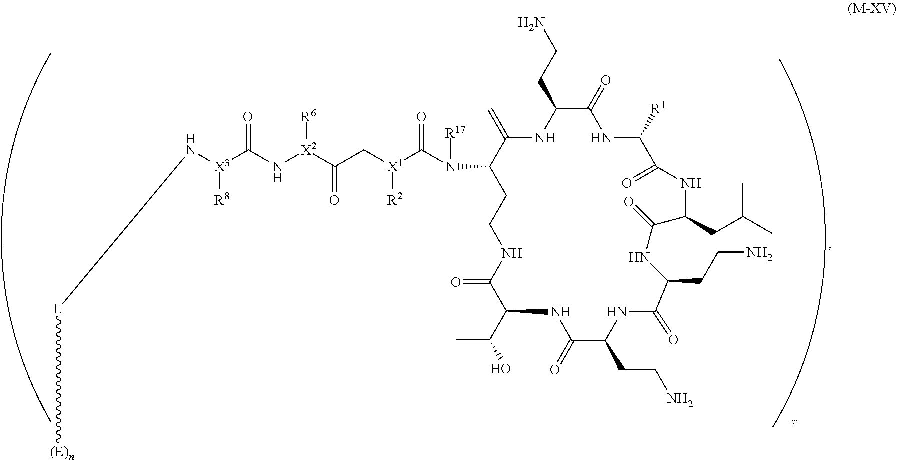

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; and each of R², R⁶, and R⁸ is, independently, H, a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; or a pharmaceutically acceptable salt thereof.

In some embodiments, the conjugate is described by formula (M-XVa):

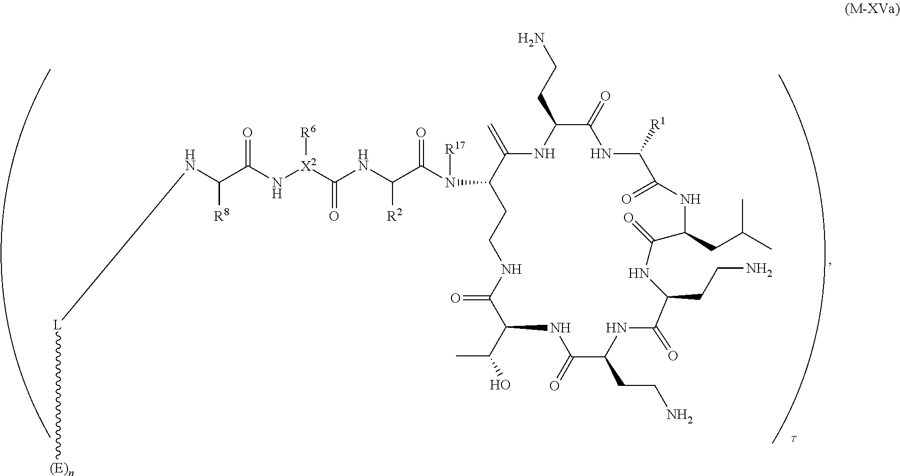

or a pharmaceutically acceptable salt thereof.

In some embodiments, the conjugate is described by formula (M-XVb):

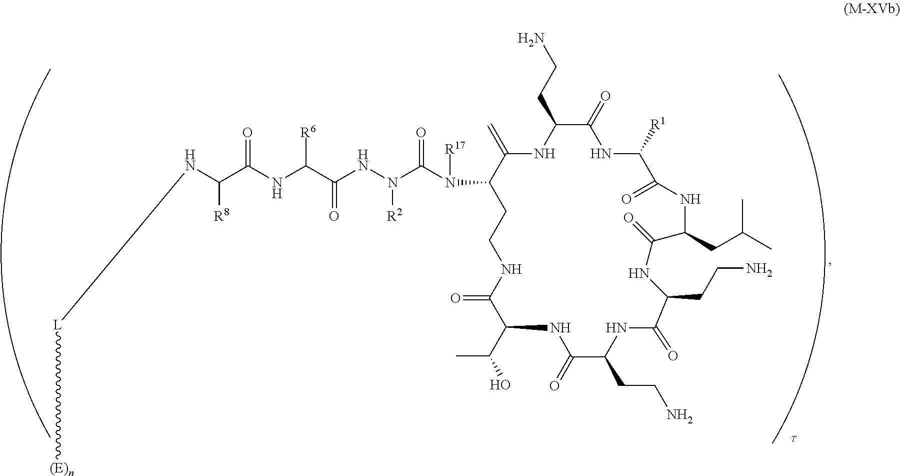

or a pharmaceutically acceptable salt thereof.

In some embodiments, the conjugate is described by formula (M-XVc):

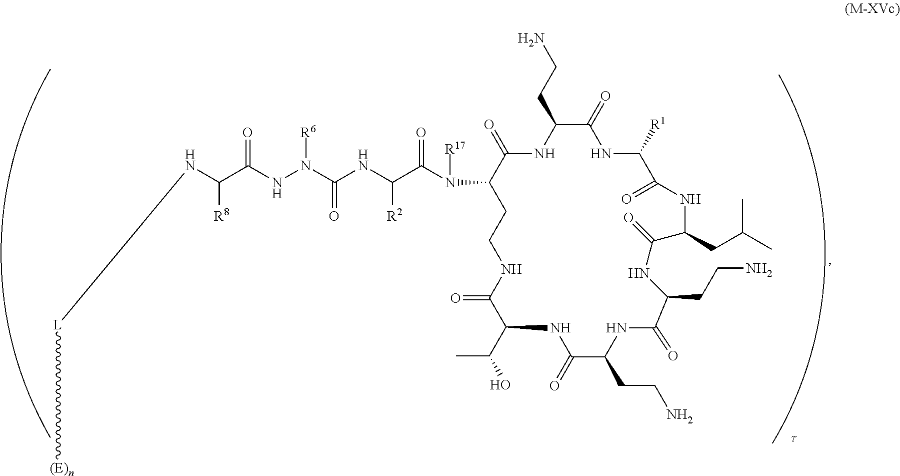

or a pharmaceutically acceptable salt thereof.

In some embodiments, the conjugate is described by formula (M-XVd):

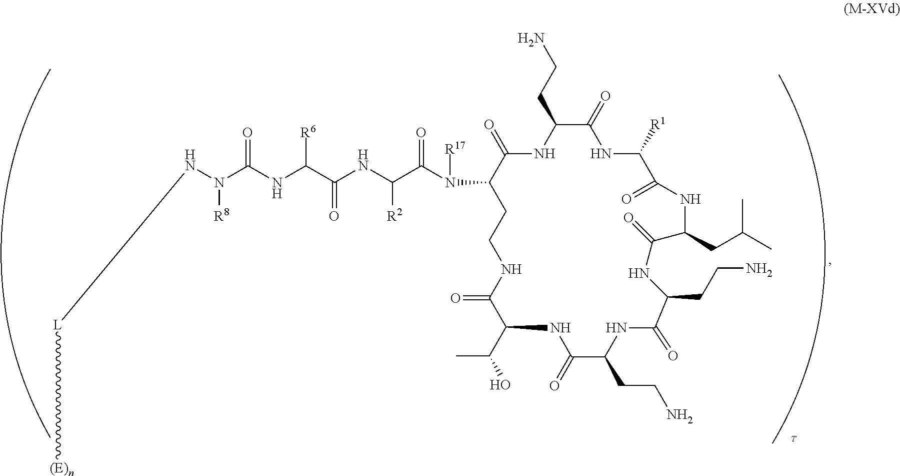

or a pharmaceutically acceptable salt thereof.

In some embodiments, the conjugate is described by formula (M-XV-1):

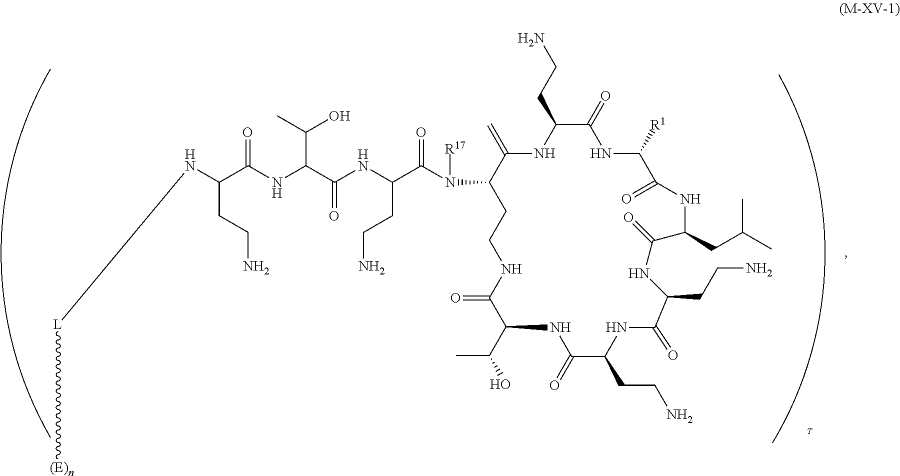

or a pharmaceutically acceptable salt thereof.

In some embodiments, the conjugate is described by formula (M-XV-1a):

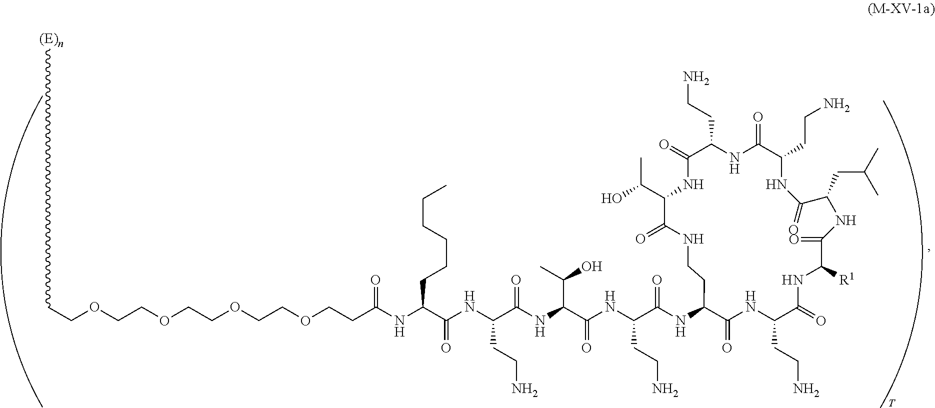

or a pharmaceutically acceptable salt thereof.

In some embodiments. R¹ is benzyl, CH₂CH(CH₃)₂, or cyclohexylmethyl.

In some embodiments, the conjugate is described by formula (M-XV-1 b):

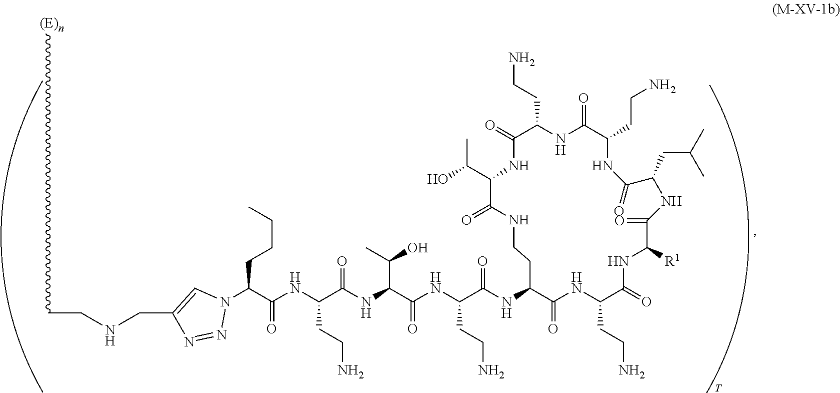

or a pharmaceutically acceptable salt thereof.

In some embodiments, the conjugate is described by formula (M-XV-1c):

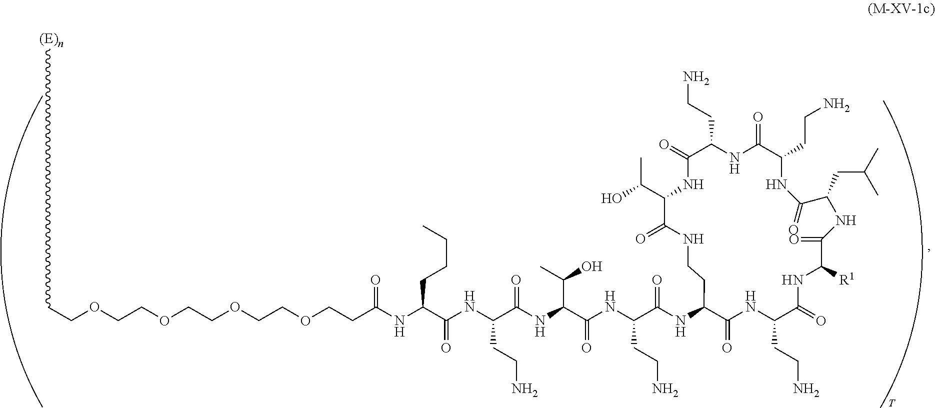

or a pharmaceutically acceptable salt thereof.

In some embodiments, the conjugate is described by formula (M-XV-1d):

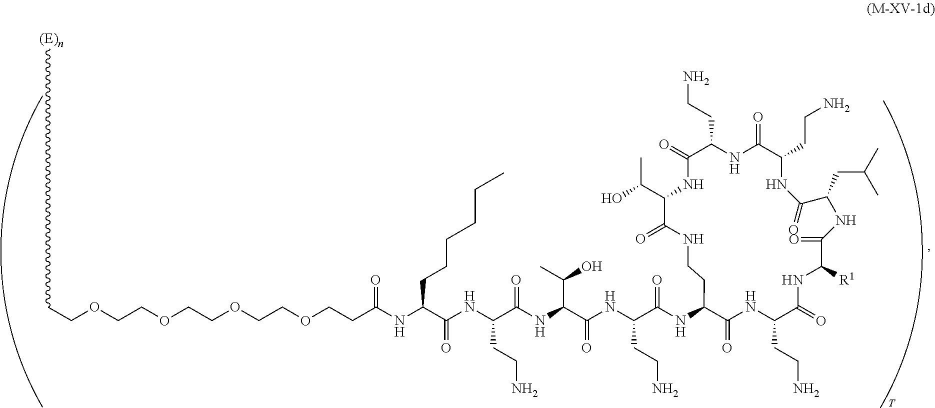

or a pharmaceutically acceptable salt thereof.

In some embodiments, the conjugate is described by formula (M-XV-2):

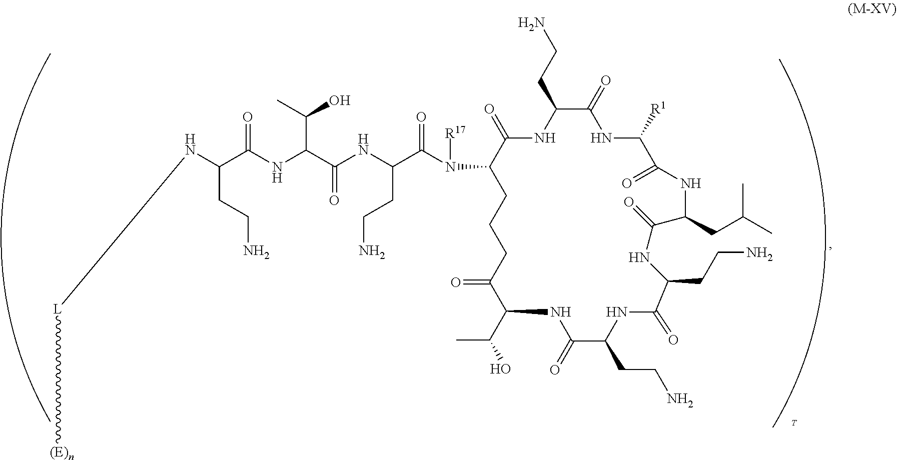

or a pharmaceutically acceptable salt thereof.

In some embodiments, the conjugate is described by formula (M-XV-3):

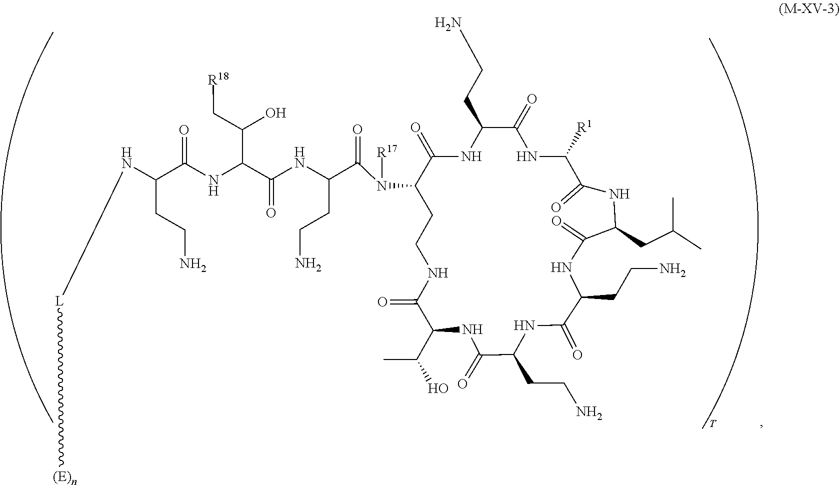

wherein R¹⁸ is a lipophilic moiety, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; or a pharmaceutically acceptable salt thereof.

In some embodiments, the conjugate is described by formula (M-XV-3a):

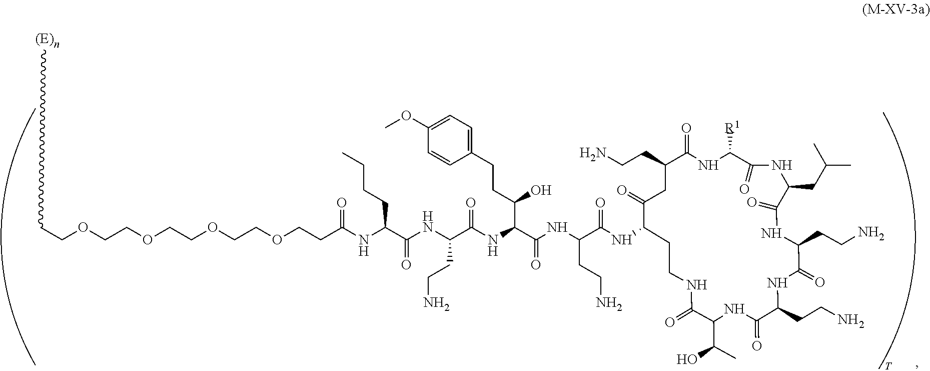

or a pharmaceutically acceptable salt thereof.

In some embodiments, the conjugate is described by formula (M-XV-3b):

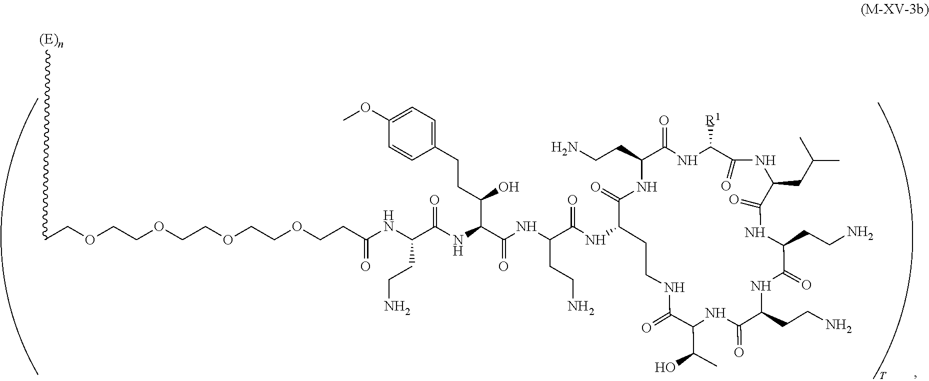

or a pharmaceutically acceptable salt thereof.

In some embodiments, the conjugate is described by formula (M-XV-3c):

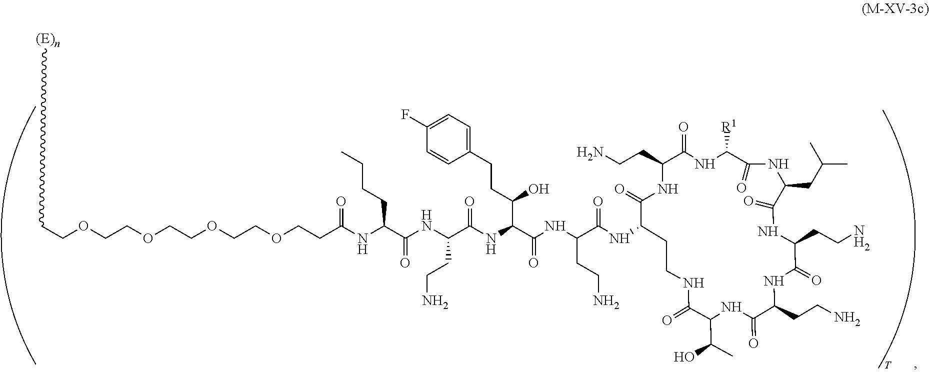

or a pharmaceutically acceptable salt thereof.

In some embodiments, the conjugate is described by formula (M-XV-3d):

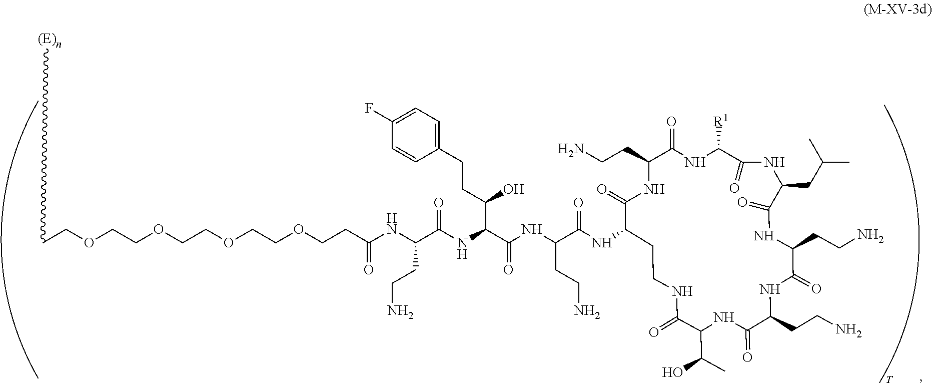

or a pharmaceutically acceptable salt thereof.

In some embodiments, the conjugate is described by formula (M-XV-3e):

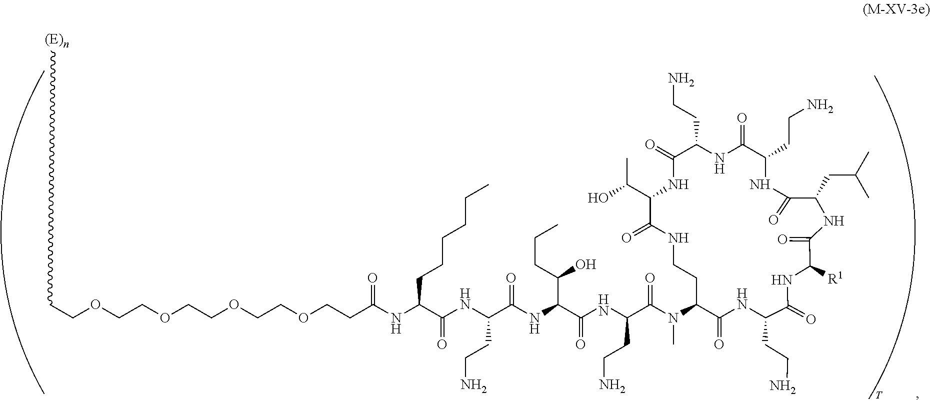

or a pharmaceutically acceptable salt thereof.

In some embodiments, the conjugate is described by formula (M-XV-3f):

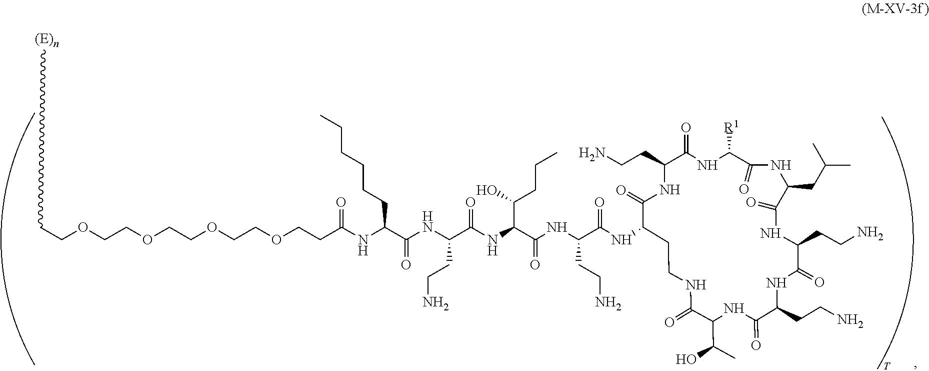

or a pharmaceutically acceptable salt thereof.

In some embodiments, the conjugate is described by formula (M-XV-3g):

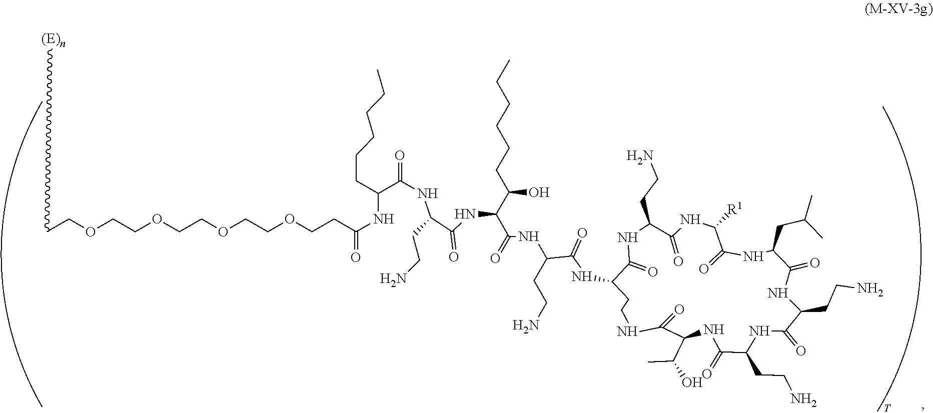

or a pharmaceutically acceptable salt thereof.

In some embodiments, the conjugate is described by formula (M-XV-3h):

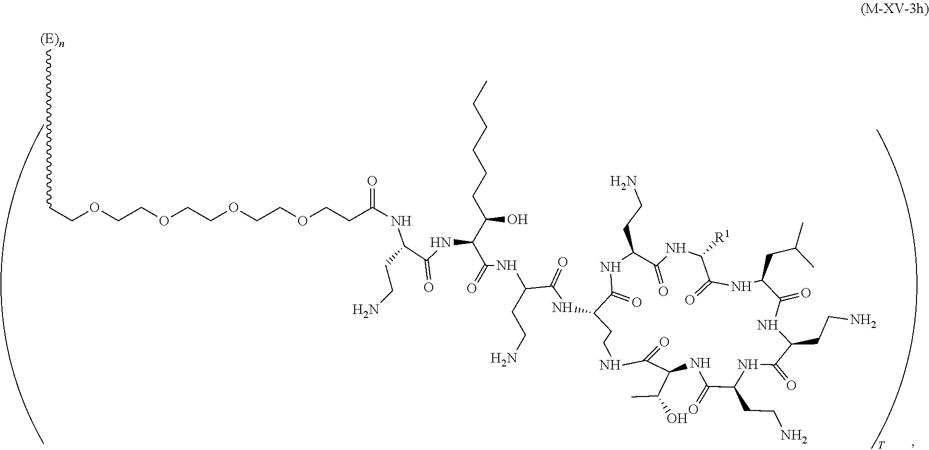

or a pharmaceutically acceptable salt thereof.

In some embodiment, the conjugate is described by formula (M-X-3i)

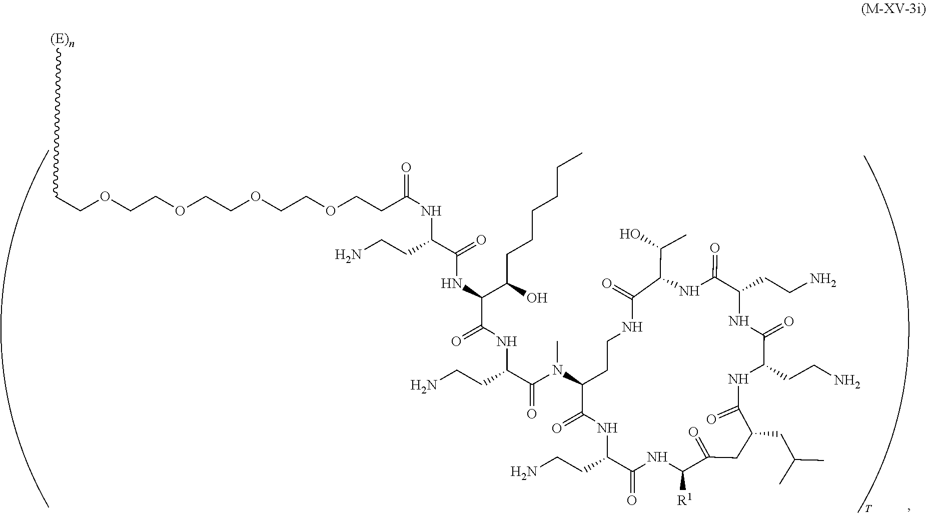

or a pharmaceutically acceptable salt thereof.

In some embodiments, the conjugate is described by formula (M-XV-3j):

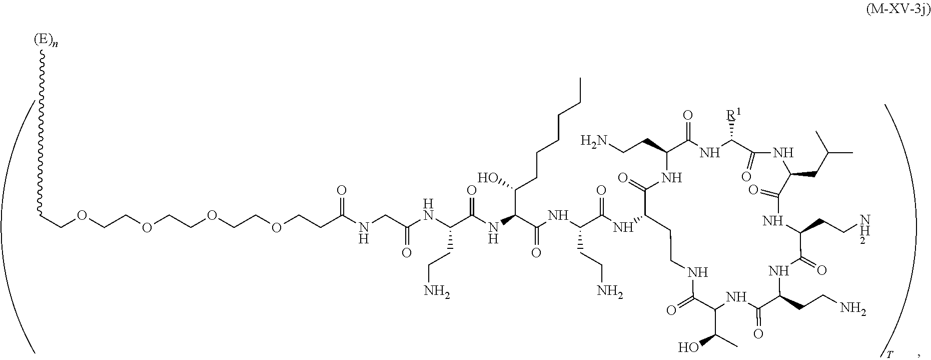

or a pharmaceutically acceptable salt thereof.

In some embodiments, the conjugate is described by formula (M-XV-3k):

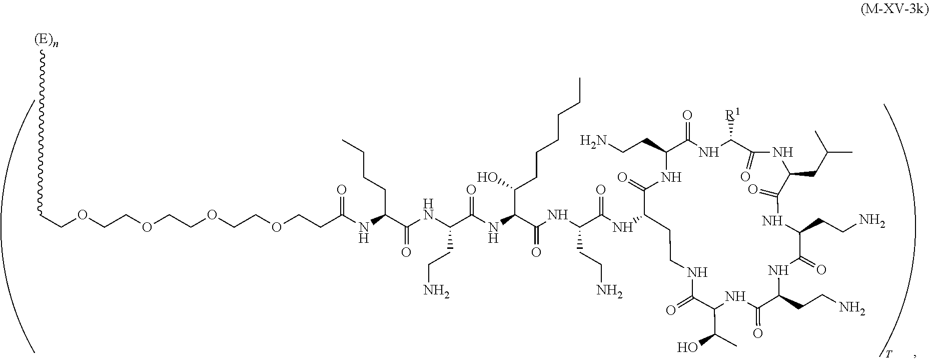

or a pharmaceutically acceptable salt thereof.

In some embodiments, the conjugate is described by formula (M-XV-3l):

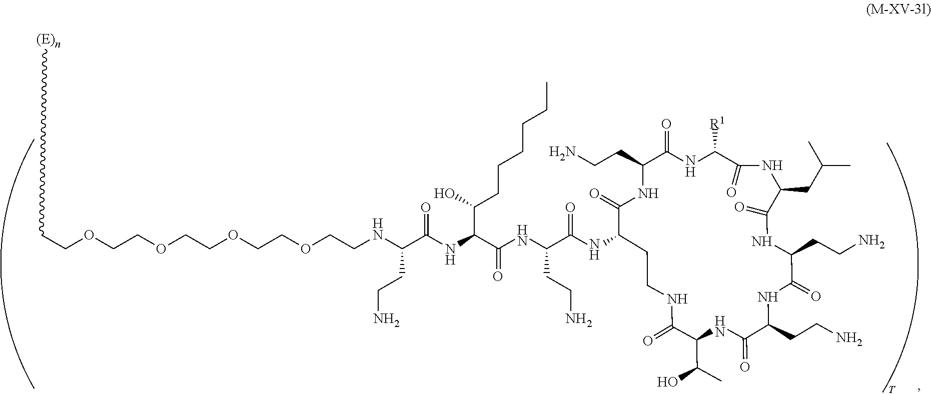

or a pharmaceutically acceptable salt thereof.

In some embodiments, the conjugate is described by formula (M-XV-3m):

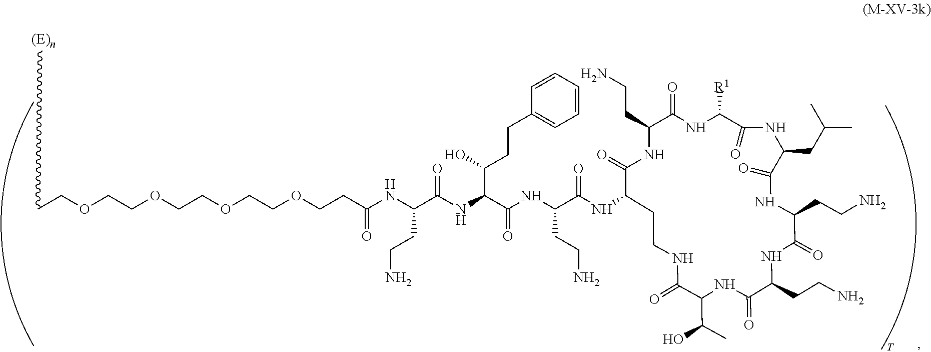

or a pharmaceutically acceptable salt thereof.

In some embodiments, the conjugate is described by formula (M-XV-3n):

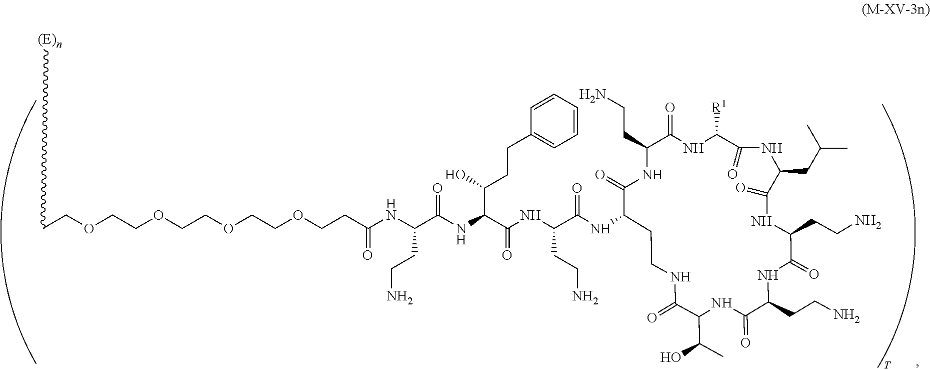

or a pharmaceutically acceptable salt thereof.

In some embodiments, the conjugate is described by formula (M-XV-3o):

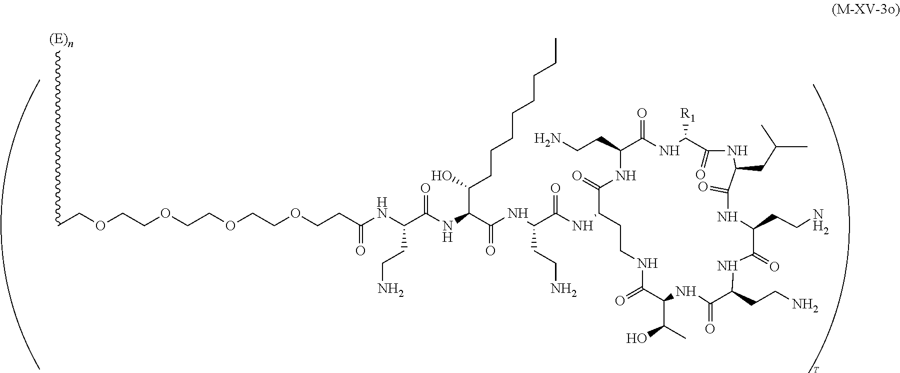

or a pharmaceutically acceptable salt thereof.

In some embodiments, the conjugate is described by formula (M-XV-3p):

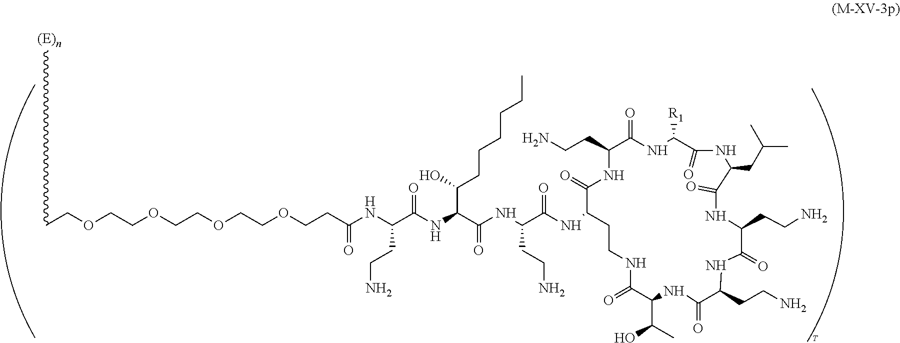

or a pharmaceutically acceptable salt thereof.

In some embodiments, the conjugate is described by formula (M-XV-3q):

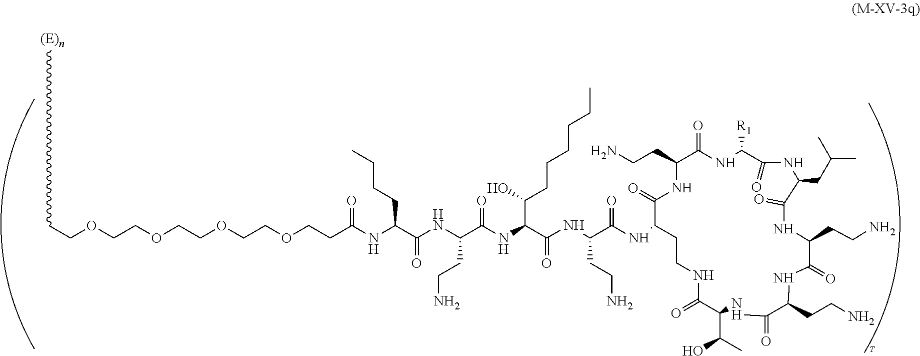

or a pharmaceutically acceptable salt thereof.

In some embodiments, the conjugate is described by formula (M-XV-3r):

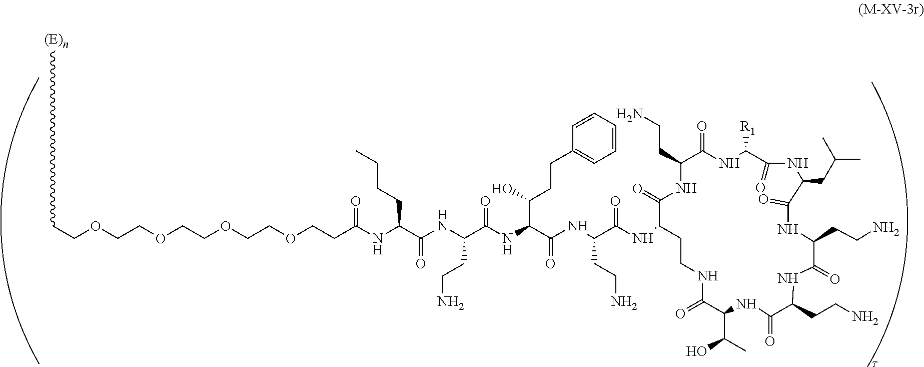

or a pharmaceutically acceptable salt thereof.

In some embodiments, the conjugate is described by formula (M-XV-3s):

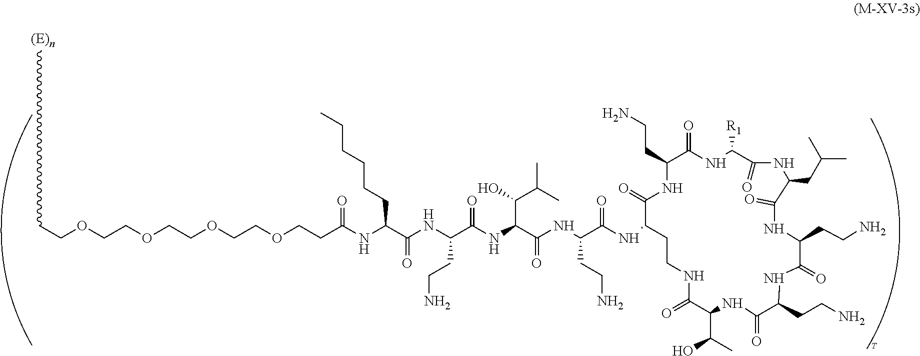

or a pharmaceutically acceptable salt thereof.

In some embodiments, the conjugate is described by formula (M-XV-3t):

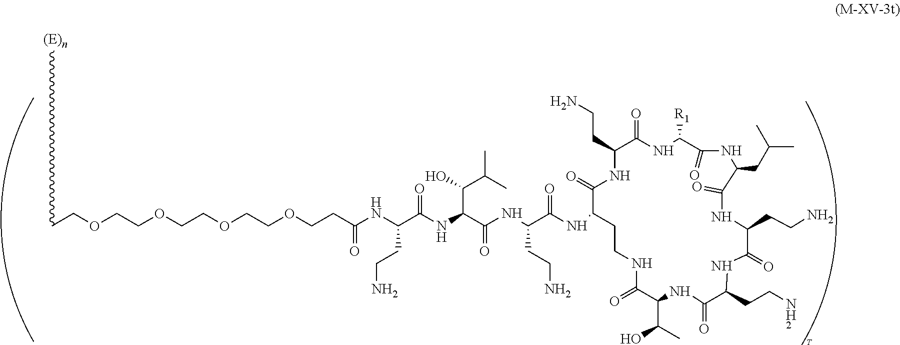

or a pharmaceutically acceptable salt thereof.

In some embodiments, the conjugate is described by formula (M-XV-3u):

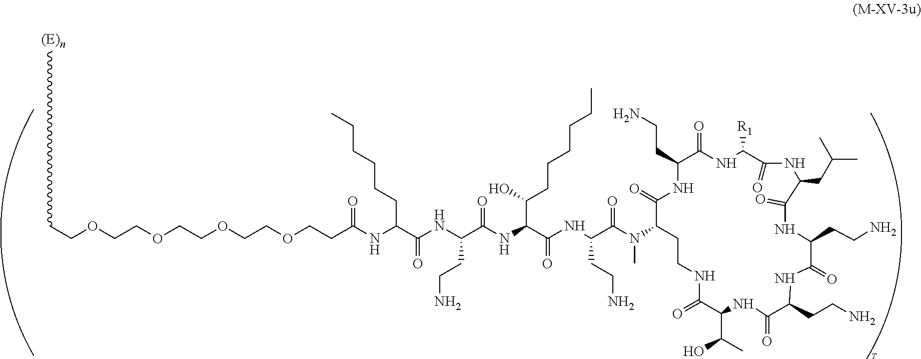

or a pharmaceutically acceptable salt thereof.

In some embodiments, the conjugate is described by formula (M-XV-3v):

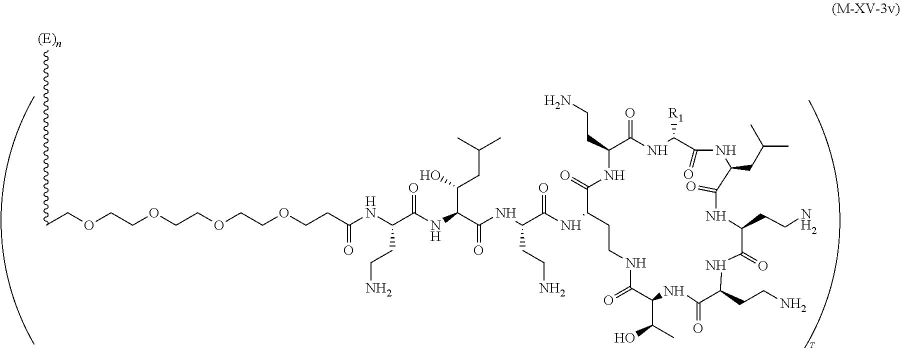

or a pharmaceutically acceptable salt thereof.

In some embodiments, the conjugate is described by formula (M-XV-3w):

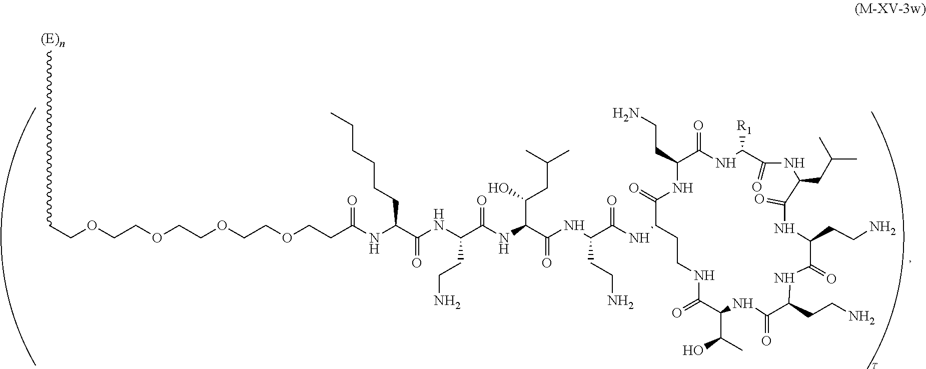

or a pharmaceutically acceptable salt thereof.

In some embodiments, the conjugate is described by formula (M-XV-3x):

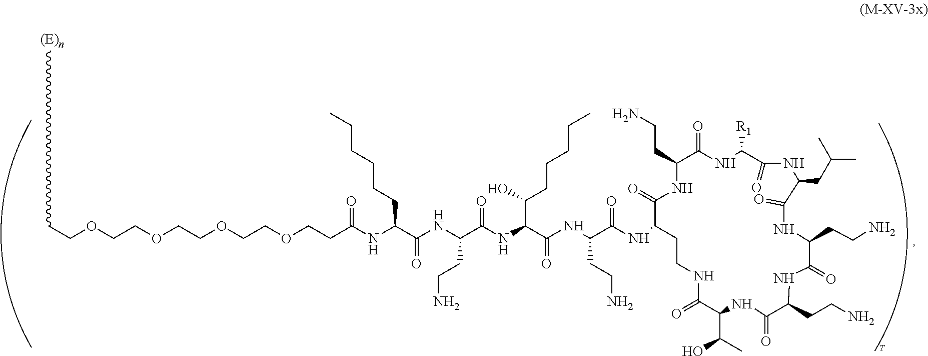

or a pharmaceutically acceptable salt thereof.

In some embodiments, the conjugate is described by formula (M-XV-3y):

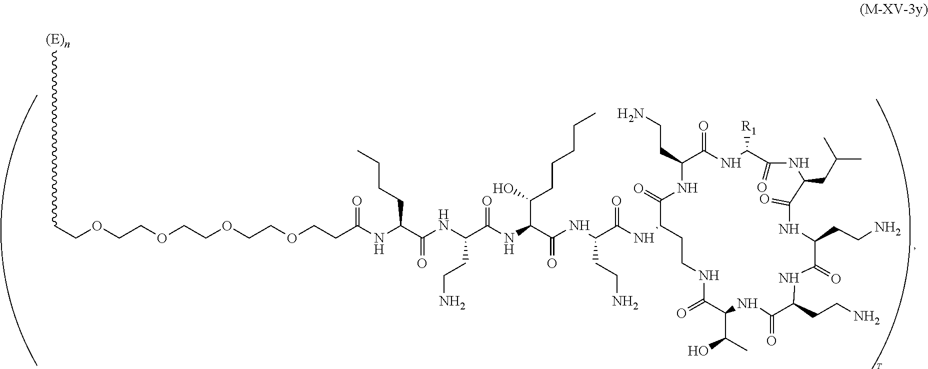

or a pharmaceutically acceptable salt thereof.

In some embodiments, the conjugate is described by formula (M-XV-3z):

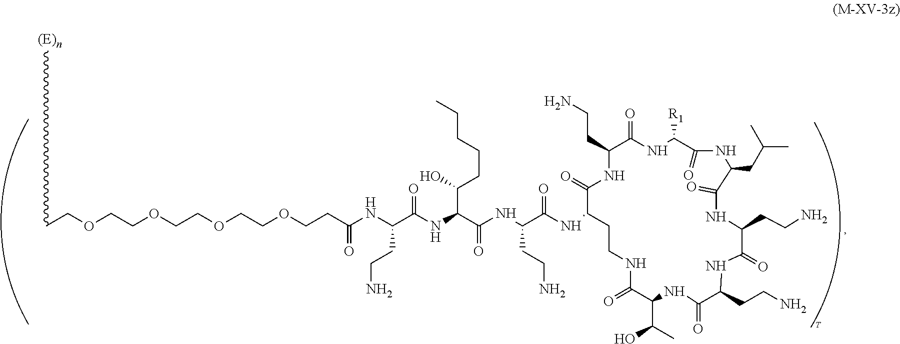

or a pharmaceutically acceptable salt thereof.

In some embodiments, the conjugate is described by formula (M-XV-3aa):

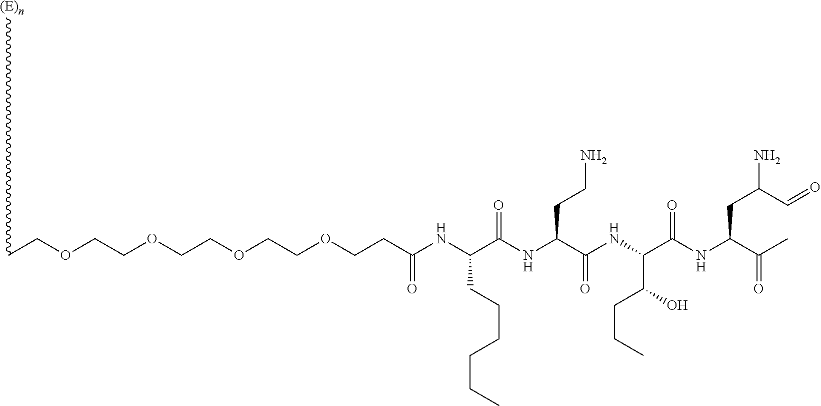

or a pharmaceutically acceptable salt thereof.

In some embodiments, the conjugate is described by formula (M-XV-3ab):

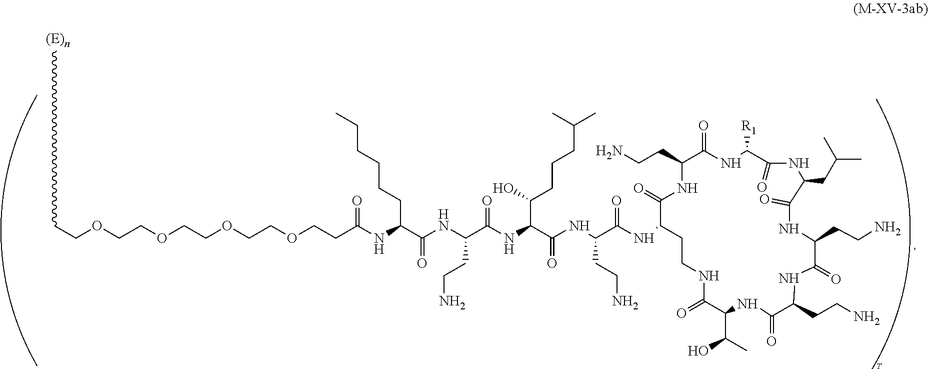

or a pharmaceutically acceptable salt thereof.

In some embodiments, the conjugate is described by formula (M-XV-3ac):

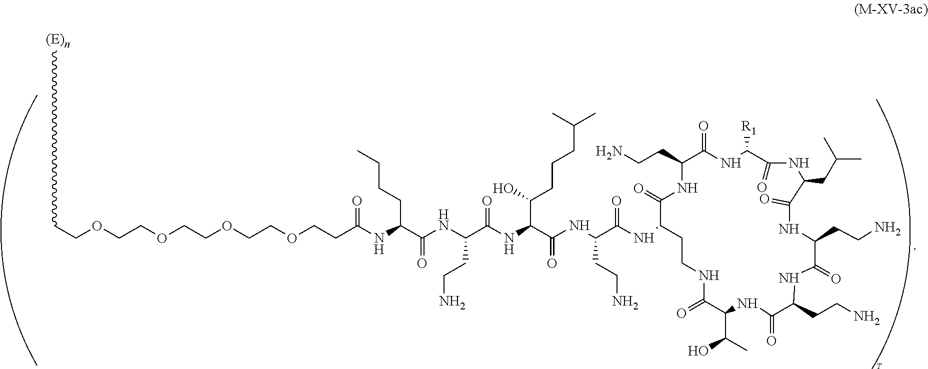

or a pharmaceutically acceptable salt thereof.

In some embodiments, the conjugate is described by formula (M-XV-3ad):

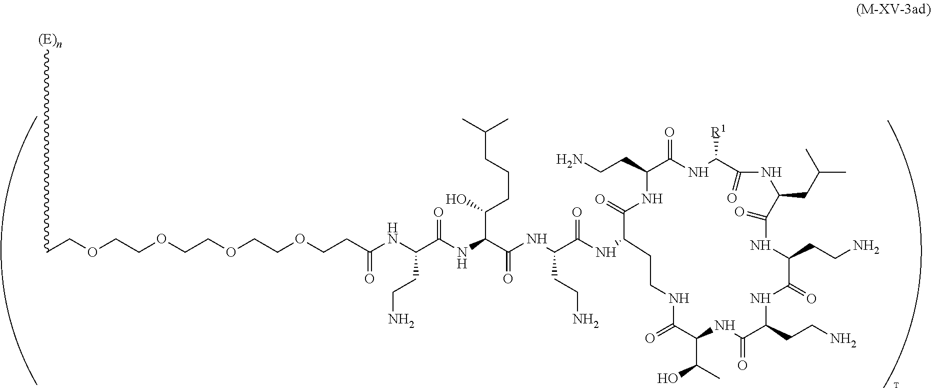

or a pharmaceutically acceptable salt thereof.

In some embodiments, the conjugate is described by formula (M-XV-3ae):

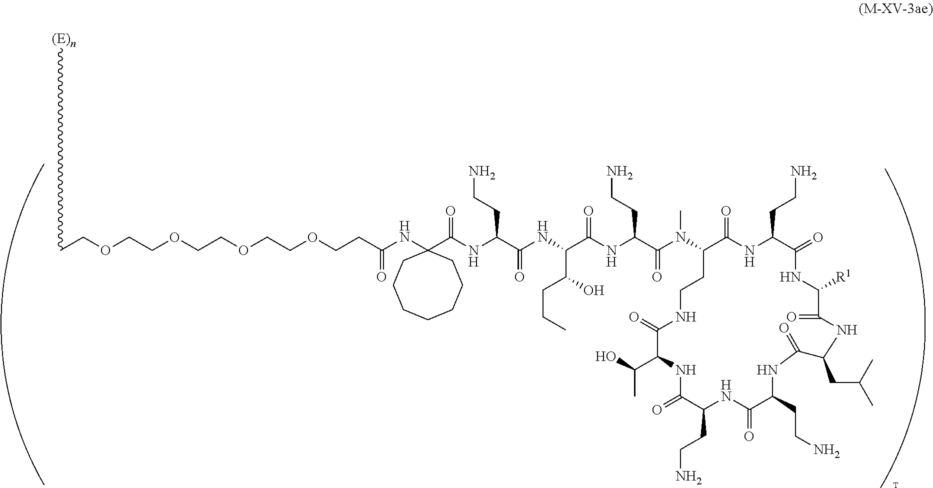

or a pharmaceutically acceptable salt thereof.

In some embodiments, the conjugate is described by formula (M-XV-4):

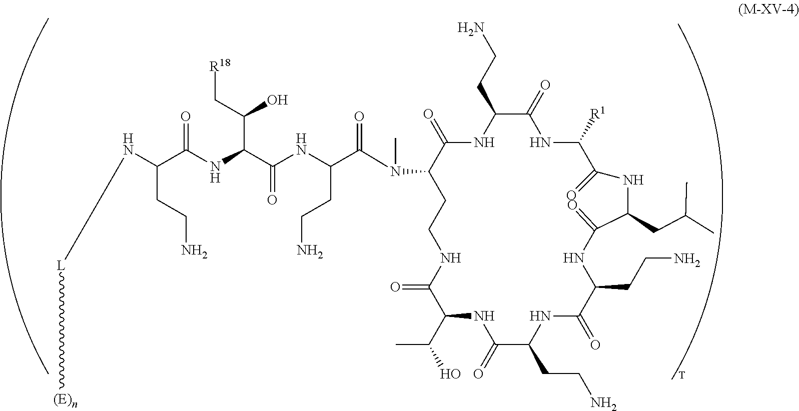

or a pharmaceutically acceptable salt thereof.

In some embodiments, the conjugate is described by formula (M-XV-5):

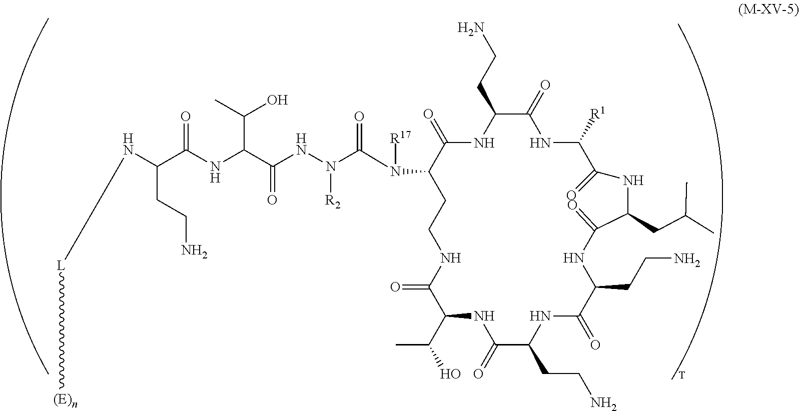

wherein R² is H or C1-C8 alkyl; or a pharmaceutically acceptable salt thereof.

In some embodiments, the conjugate is described by formula (M-XV-5a):

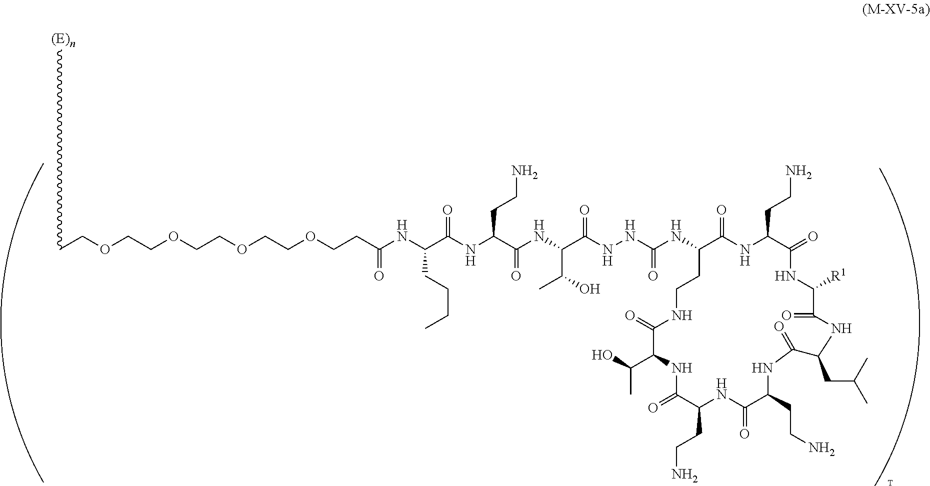

or a pharmaceutically acceptable salt thereof.

In some embodiments, the conjugate is described by formula (M-XV-6):

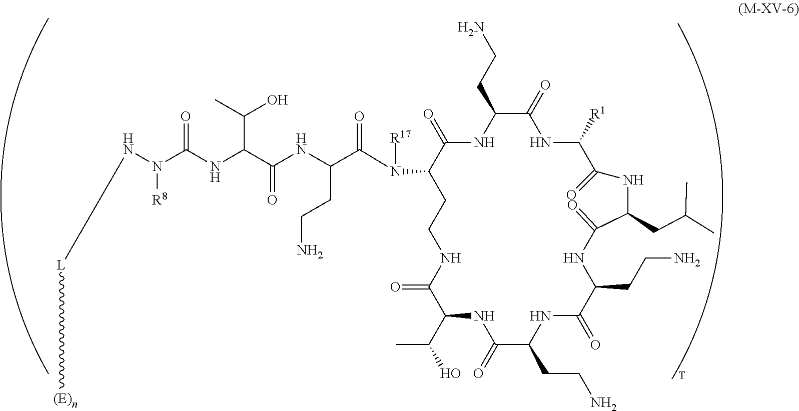

wherein R⁸ is H or C1-C8 alkyl; or a pharmaceutically acceptable salt thereof.

In some embodiments, the conjugate is described by formula (M-XV-7):

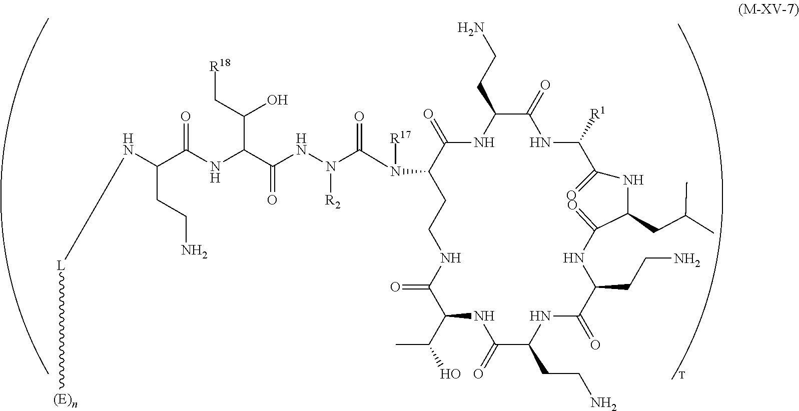

wherein R⁸ is a lipophilic moiety, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C8-C35 alkaryl, or optionally substituted C6-C35 heteroalkaryl; R² is H or C1-C8 alkyl; or a pharmaceutically acceptable salt thereof.

In some embodiments, the conjugate is described by formula (M-XV-7a):

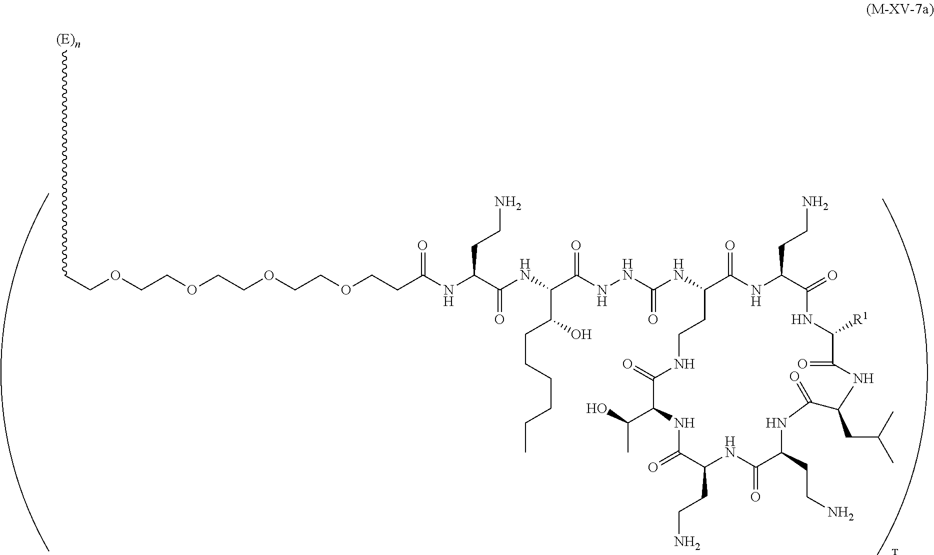

or a pharmaceutically acceptable salt thereof.

In some embodiments, the conjugate is described by formula (M-XV-7b):

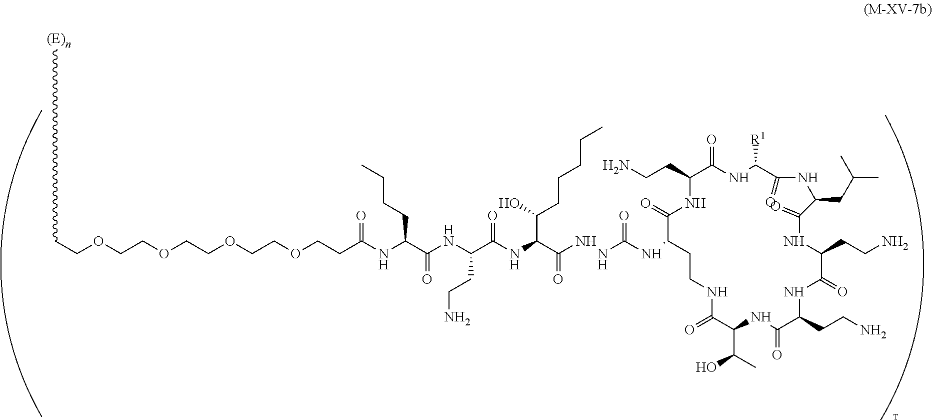

or a pharmaceutically acceptable salt thereof.

In some embodiments, the conjugate is described by formula (M-XV-8):

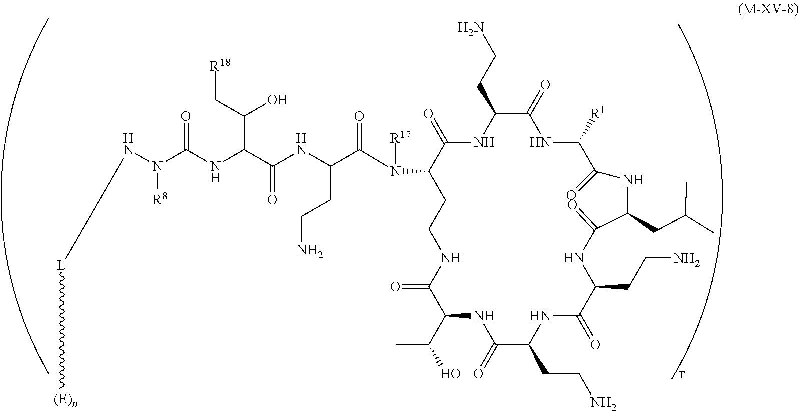

wherein R¹⁸ is a lipophilic moiety, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; and R⁸ is H or C1-C8 alkyl; or a pharmaceutically acceptable salt thereof.

In some embodiments, the conjugate is described by formula (M-XV-9):

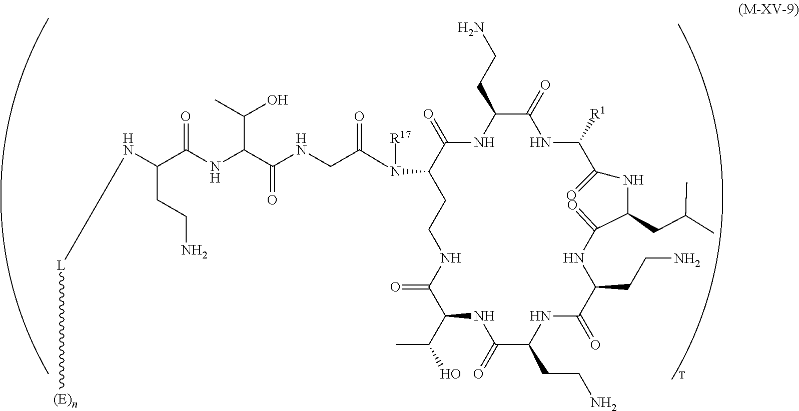

or a pharmaceutically acceptable salt thereof.

In some embodiments, the conjugate is described by formula (M-XV-9a):

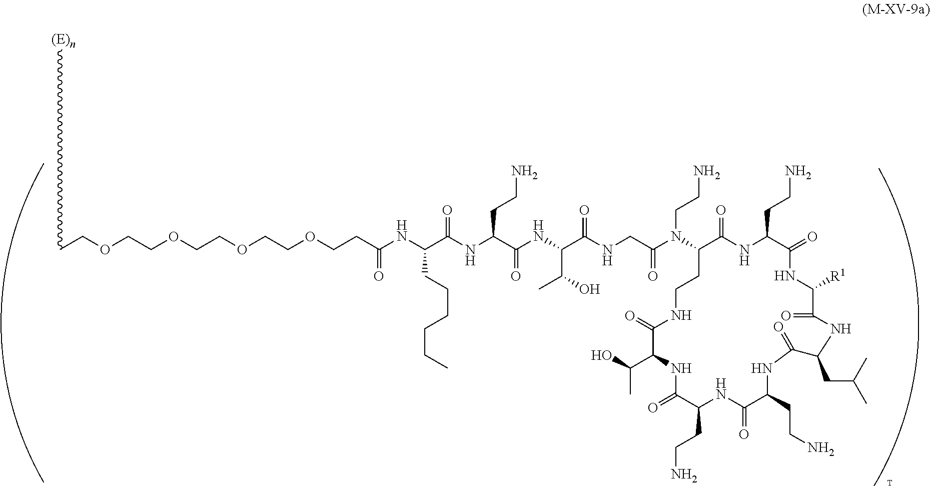

or a pharmaceutically acceptable salt thereof.

In some embodiments of this aspect, the conjugate is described by formula (M-XVI):

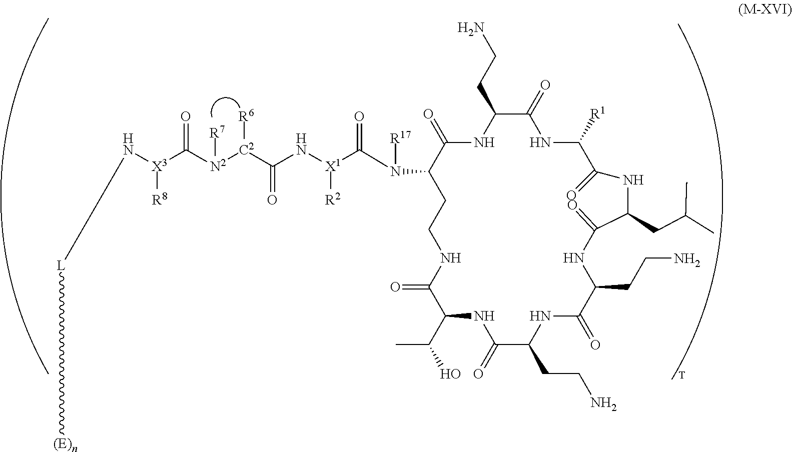

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R² and R⁸ is, independently, a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; and each R⁶, R⁷, N², and C² together form an optionally substituted 5-8 membered ring comprising optionally substituted C3-C7 heterocycloalkyl comprising a nitrogen atom and additional 0-2 heteroatoms independently selected from N, O, and S, or optionally substituted C2-C7 heteroaryl comprising a nitrogen atom and additional 0-2 heteroatoms independently selected from N, O, and S; or a pharmaceutically acceptable salt thereof.

In some embodiments, the conjugate is described b formula M-XVIa):

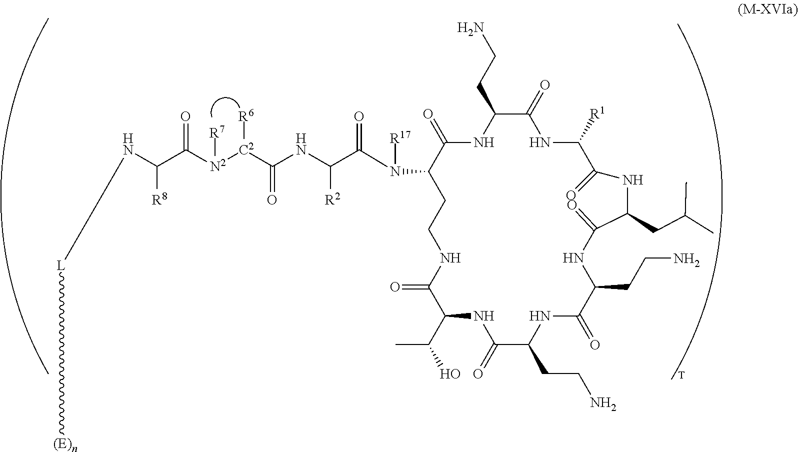

or a pharmaceutically acceptable salt thereof.

In some embodiments, the conjugate is described by formula (M-XVIb):

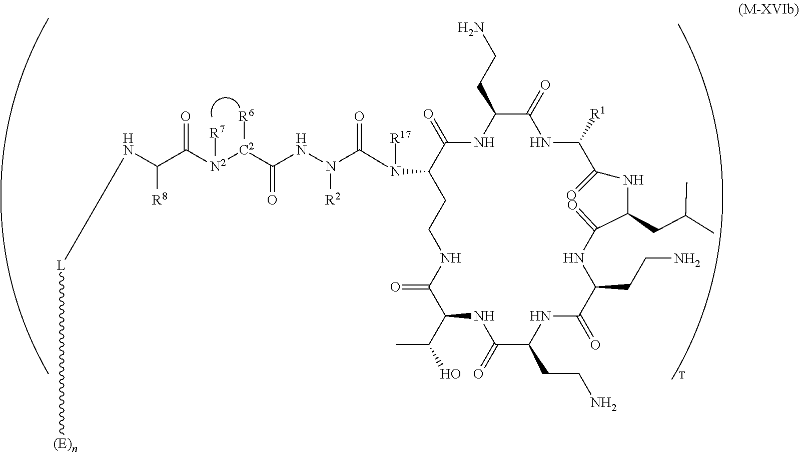

or a pharmaceutically acceptable salt thereof.

In some embodiments, the conjugate is described by formula (M-XVIc):

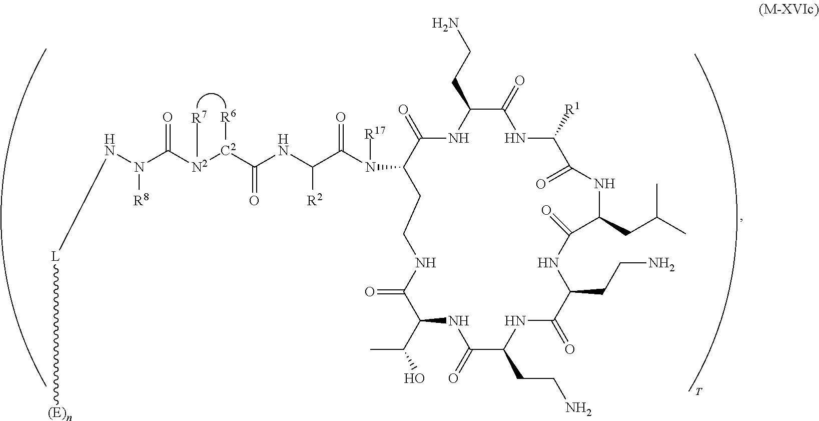

or a pharmaceutically acceptable salt thereof.

In some embodiments of this aspect, the conjugate is described by formula (M-XVII):

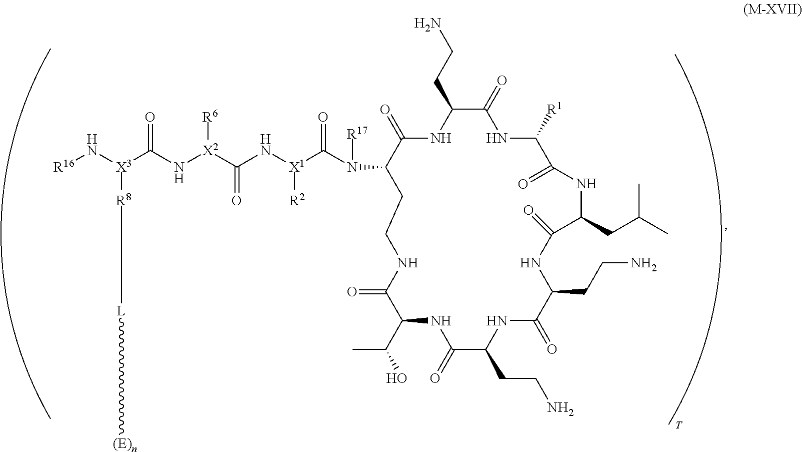

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R² and R⁸ is, independently, a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; each R⁸ is optionally substituted C1-C20 alkylene, optionally substituted C1-C20 heteroalkylene, optionally substituted C3-C20 cycloalkylene, optionally substituted C3-C20 heterocycloalkylene, optionally substituted C4-C20 heterocycloalkenylene, optionally substituted C8-C20 heterocycloalkynylene, optionally substituted C5-C15 arylene, or optionally substituted C2-C15 heteroarylene; and each R¹⁶ is H, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments, the conjugate is described by formula (M-XVIIa):

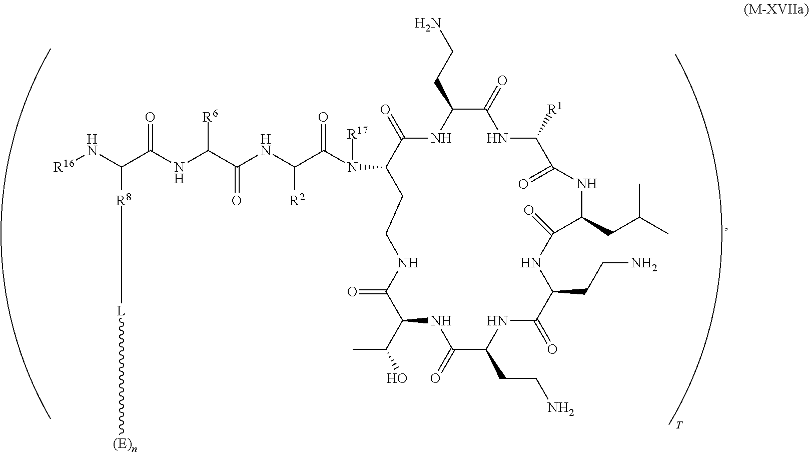

or a pharmaceutically acceptable salt thereof.

In some embodiments, the conjugate is described by formula (M-XVIIb):

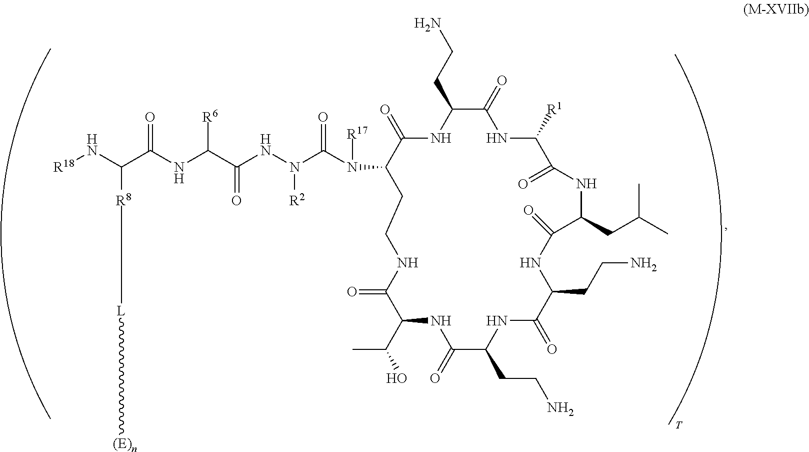

or a pharmaceutically acceptable salt thereof.

In some embodiments, the conjugate is described by formula (M-XVIIc):

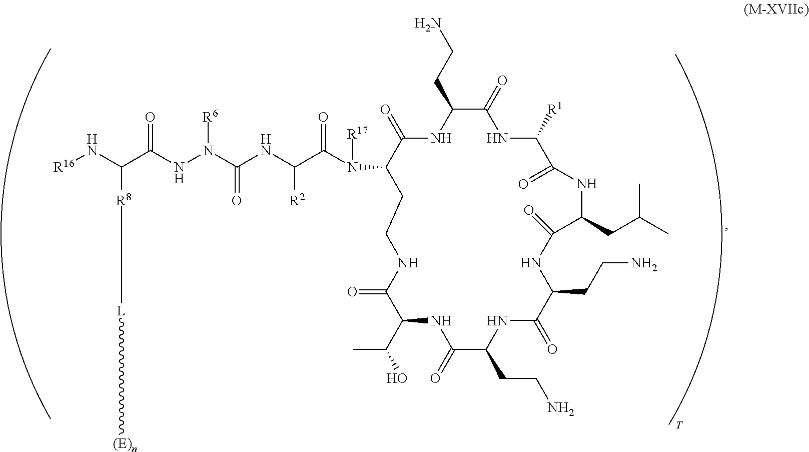

or a pharmaceutically acceptable salt thereof.

In some embodiments, the conjugate is ae described by formula (M-XVIId):

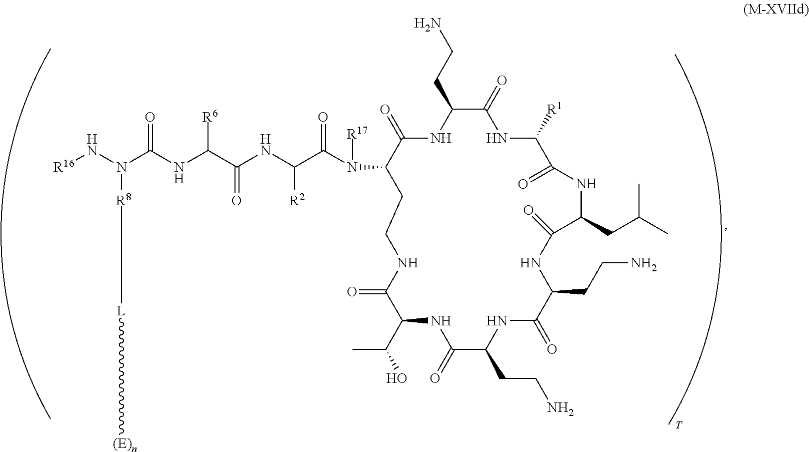

or a pharmaceutically acceptable salt thereof.

In some embodiments, the conjugate is described by formula (M-XVII-1):

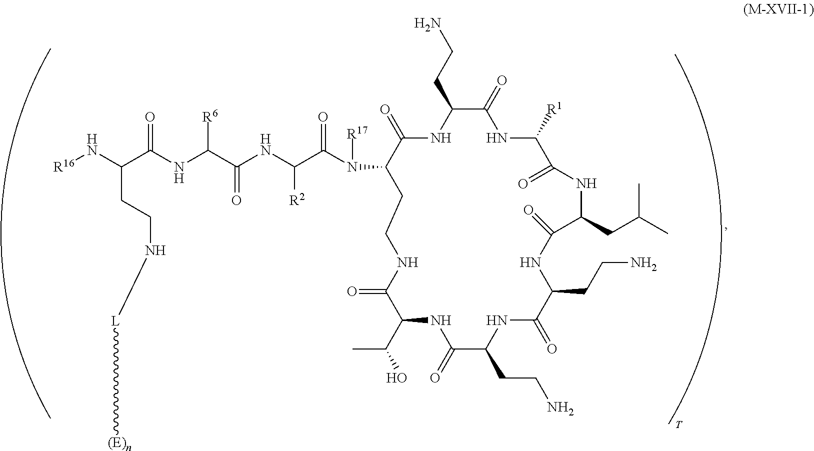

or a pharmaceutically acceptable salt thereof.

In some embodiments, the conjugate is described by formula (M-XVII-2):

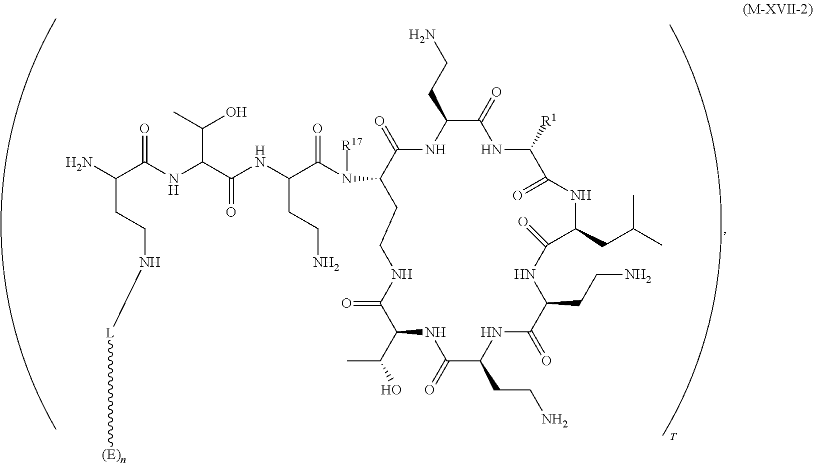

or a pharmaceutically acceptable salt thereof.

In some embodiments the conjugate is described by formula M-XVII-3):

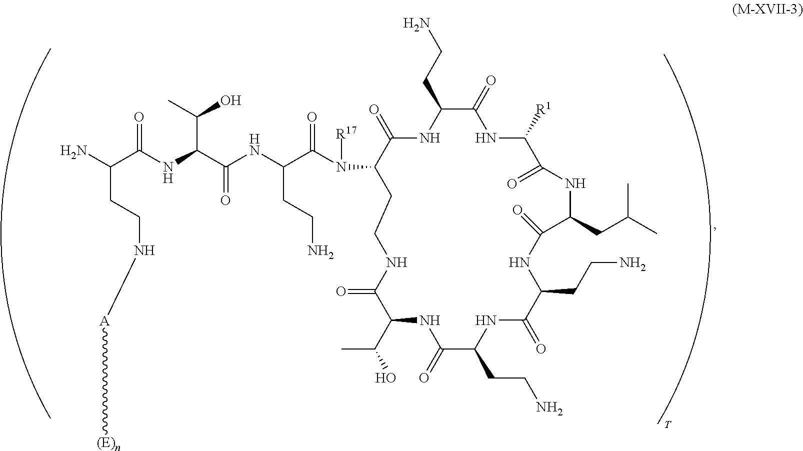

or a pharmaceutically acceptable salt thereof.

In some embodiments, the conjugate is described by formula (M-XVII-4):

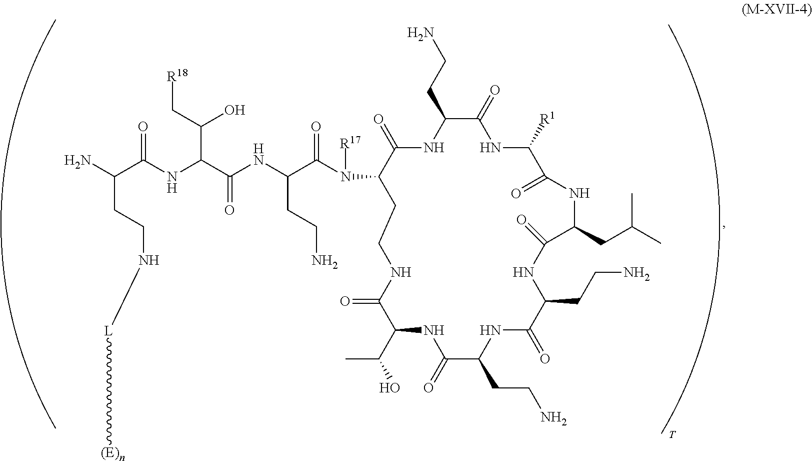

wherein R¹⁸ is a lipophilic moiety, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; or a pharmaceutically acceptable salt thereof.

In some embodiments, the conjugate is described by formula (M-XVII-5):

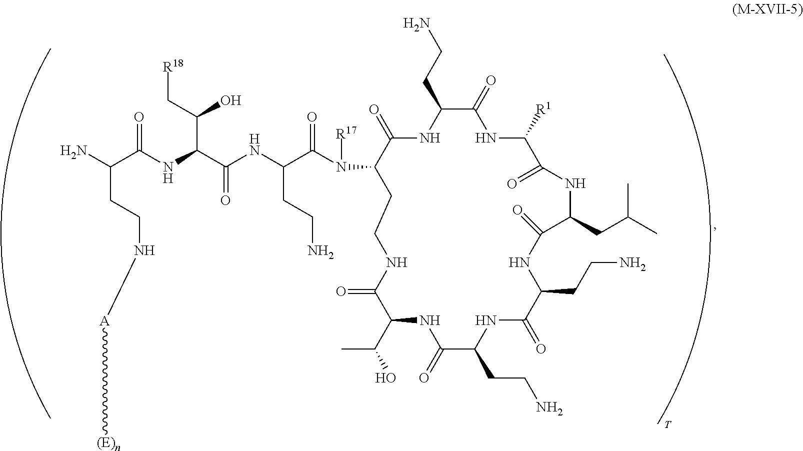

or a pharmaceutically acceptable salt thereof.

In some embodiments, the conjugate is described by formula (M-XVII-6):

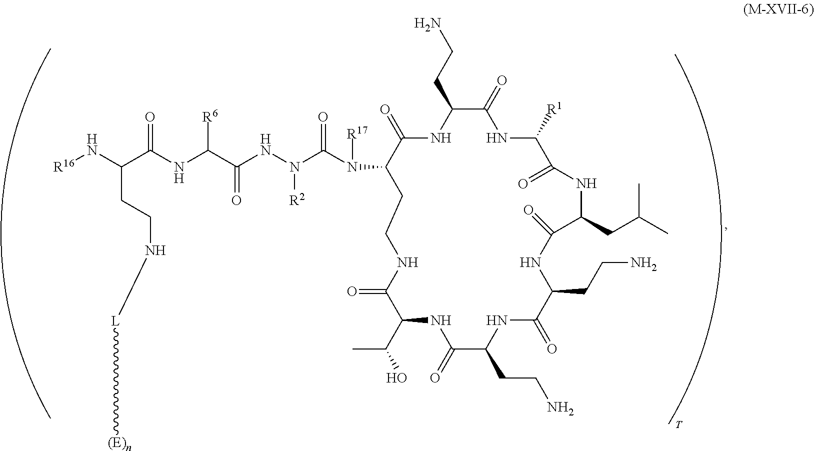

wherein R² is H or C1-C8 alkyl; or a pharmaceutically acceptable salt thereof.

In some embodiments, the conjugate is described by formula (M-XVII-7):

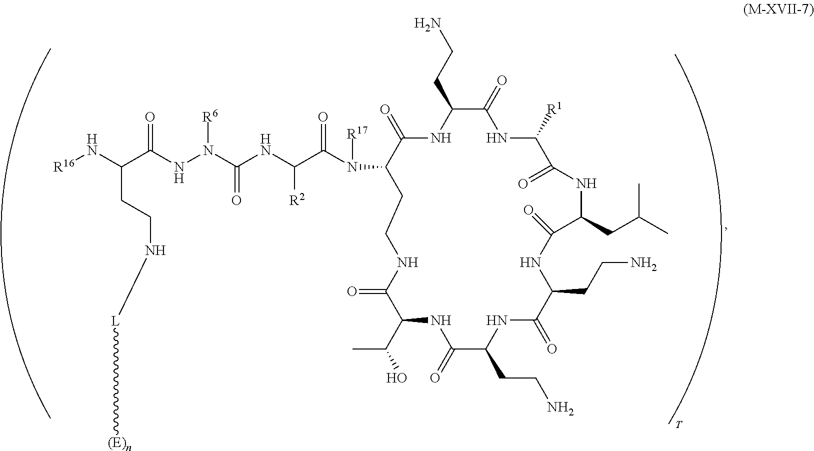

wherein R⁶ is H or C1-C8 alkyl; or a pharmaceutically acceptable salt thereof.

In some embodiments, the conjugate is described by formula (M-XVII-8):

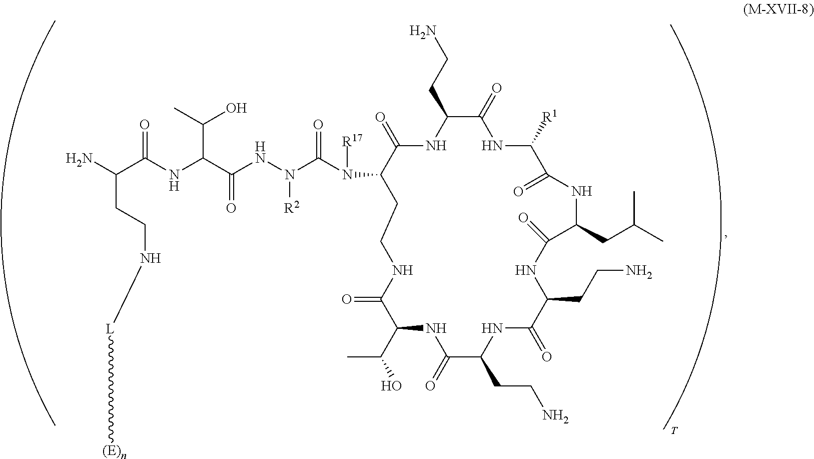

wherein R² is H or C1-C8 alkyl; or a pharmaceutically acceptable salt thereof.

In some embodiments, the conjugate is described by formula (M-XVII-9):

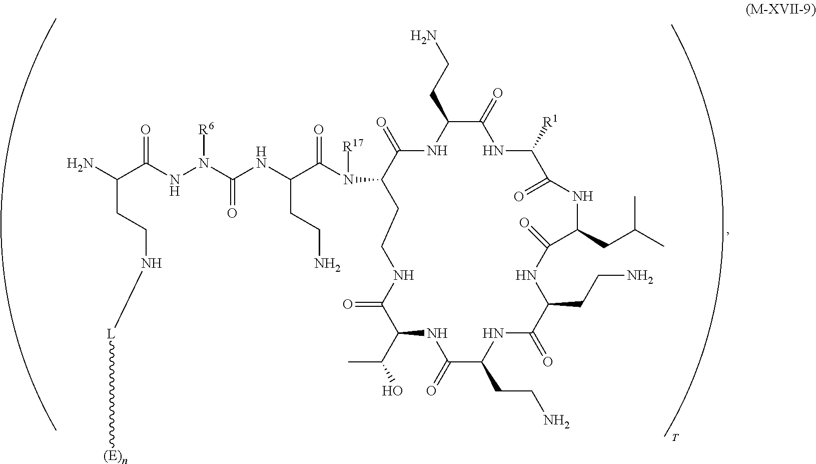

wherein R⁶ is H or C1-C8 alkyl; or a pharmaceutically acceptable salt thereof.

In some embodiments, the conjugate is described by formula (M-XVII-10):

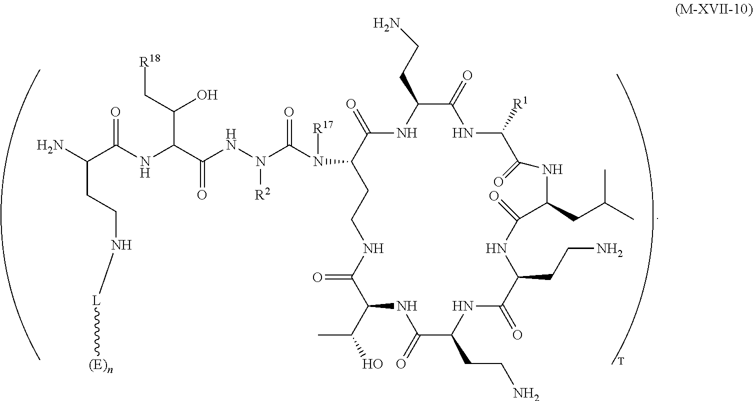

wherein R² is H or C1-C8 alkyl; and R¹⁸ is a lipophilic moiety, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; or a pharmaceutically acceptable salt thereof.

In some embodiments, the conjugate is described by formula (M-XVII-11):

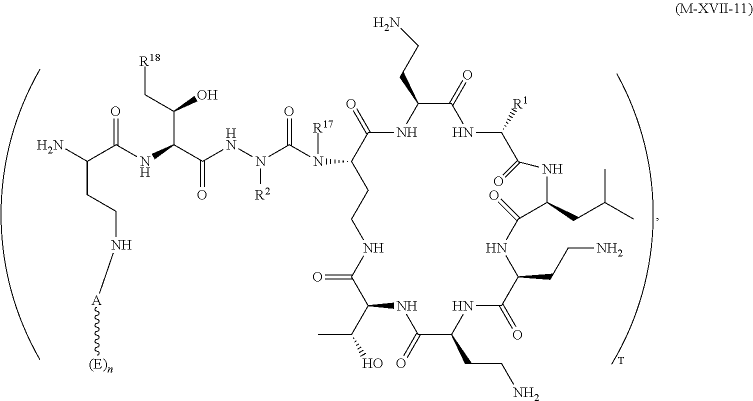

or a pharmaceutically acceptable salt thereof.

In some embodiments, the conjugate is described by formula (M-XVIII):

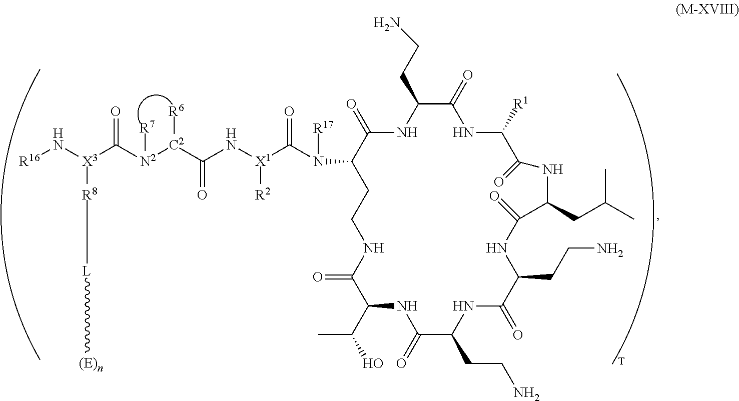

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each R² is a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; each R⁶, R⁷, N², and C² together form an optionally substituted 5-8 membered ring comprising optionally substituted C3-C7 heterocycloalkyl comprising a nitrogen atom and additional 0-2 heteroatoms independently selected from N, O, and S, or optionally substituted C2-C7 heteroaryl comprising a nitrogen atom and additional 0-2 heteroatoms independently selected from N, O, and S; each R⁸ is optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene; and each R¹⁶ is H, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or a pharmaceutically acceptable salt thereof.

In some embodiments, the conjugate is described by formula (M-XVIIIa):

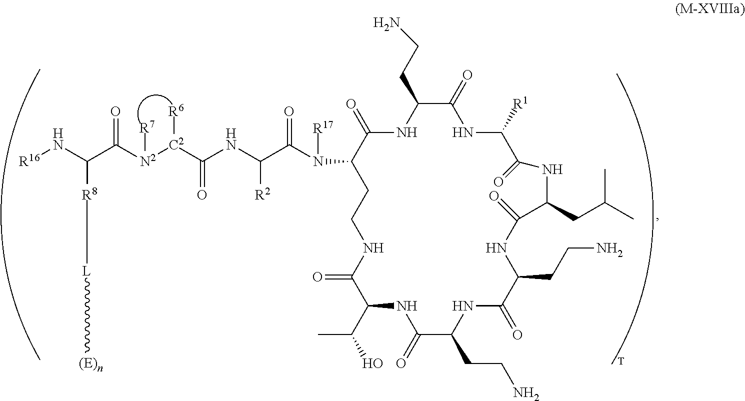

or a pharmaceutically acceptable salt thereof.

In some embodiments, the conjugate is described by formula (M-XVIIIb):

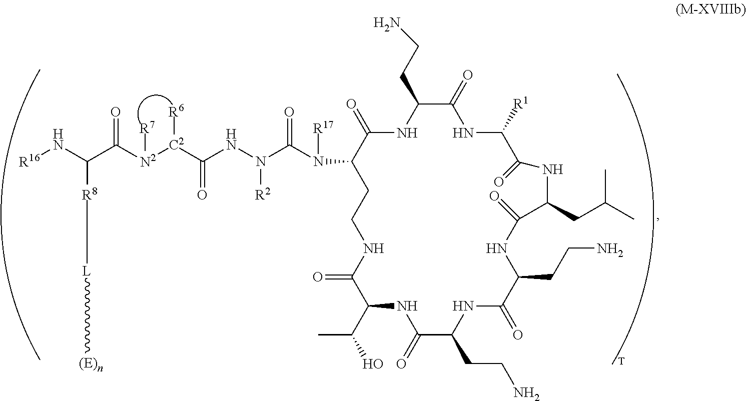

or a pharmaceutically acceptable salt thereof.

In some embodiments, the conjugate is described by formula (M-XVIIIc):

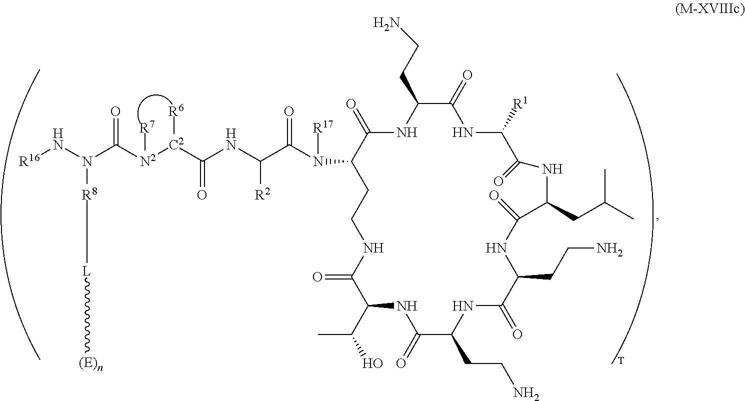

or a pharmaceutically acceptable salt thereof.

In some embodiments, the conjugate is described by formula (M-XXVII):

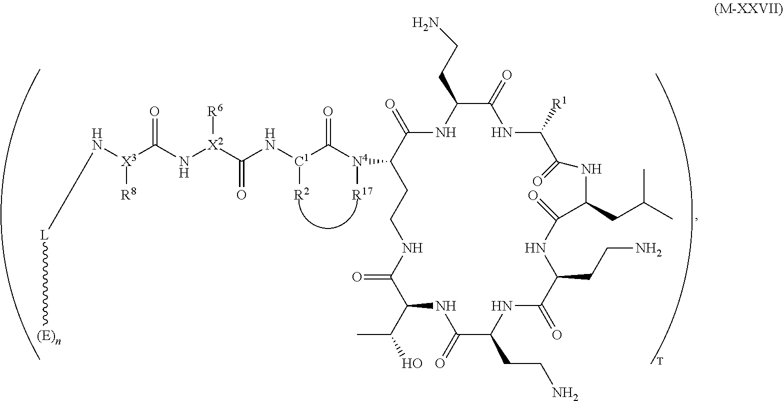

wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; each of R⁶ and R⁸ is, independently, a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; and each R², R¹⁷, N⁴, and C¹ together form an optionally substituted 5-8 membered ring comprising optionally substituted C3-C7 heterocycloalkyl comprising a nitrogen atom and additional 0-2 heteroatoms independently selected from N, O, and S, or optionally substituted C2-C7 heteroaryl comprising a nitrogen atom and additional 0-2 heteroatoms independently selected from N, O, and S; or a pharmaceutically acceptable salt thereof.

In some embodiments, the conjugate is described by formula (M-XXVIIa):

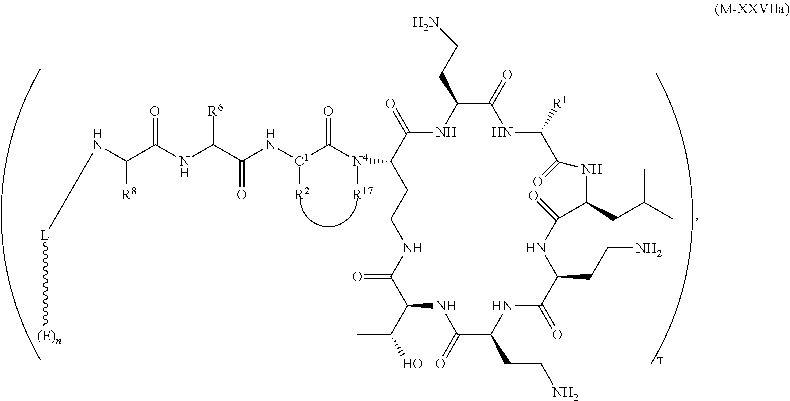

or a pharmaceutically acceptable salt thereof.

In some embodiments, the conjugate is described by formula (M-XXVIIb):

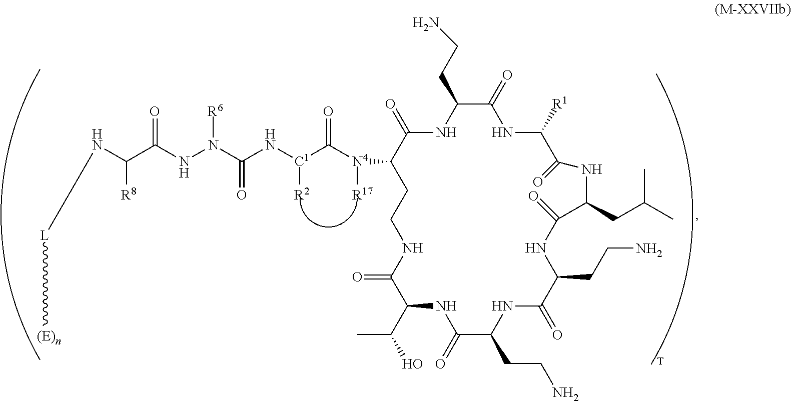

or a pharmaceutically acceptable salt thereof.

In some embodiments, the conjugate is described by formula (M-XXVIIc):

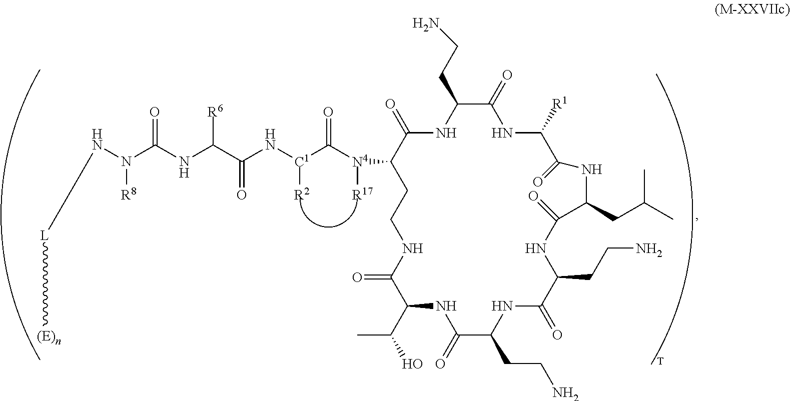

or a pharmaceutically acceptable salt thereof.

In some embodiments, the conjugate is described by formula (M-XXVII-1):

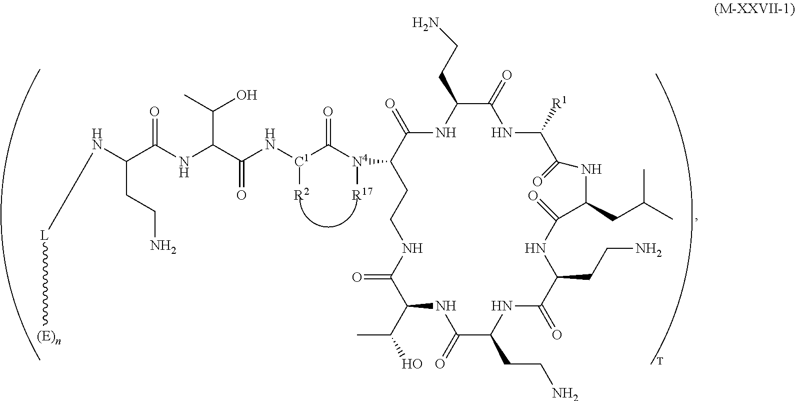

or a pharmaceutically acceptable salt thereof.

In some embodiments, the conjugate is described by formula (M-XXVI-1a):

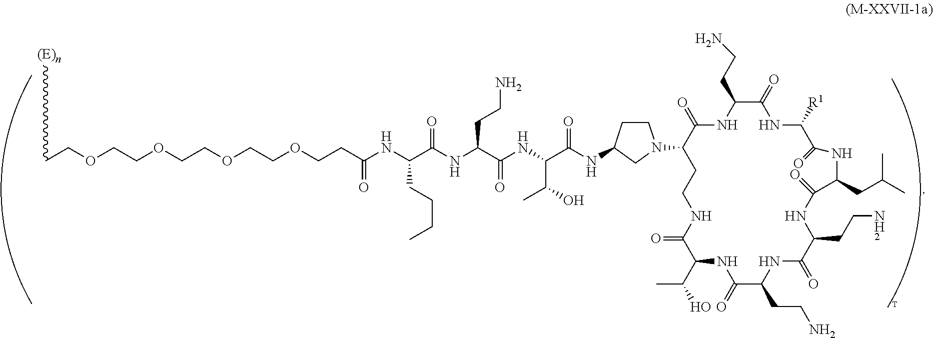

or a pharmaceutically acceptable salt thereof.

In some embodiments, each R² is optionally substituted C1-C5 alkamino (e.g., CH₂NH₂ or CH₂CH₂NH₂) or a polar moiety, or a pharmaceutically acceptable salt thereof. In some embodiments, each R^{e} is a polar moiety, or a pharmaceutically acceptable salt thereof. In some embodiments, the polar moiety comprises C1-C4 hydroxylalkyl group, a carboxylic acid group, an ester group, or an amide group, or a pharmaceutically acceptable salt thereof. In some embodiments, the polar moiety is C1-C4 hydroxyalkyl, or a pharmaceutically acceptable salt thereof. In some embodiments, the polar moiety is CHCH₃OH or CH₂OH, or a pharmaceutically acceptable salt thereof.

In some embodiments, each R⁸ is optionally substituted C1-C5 alkamino or optionally substituted C5-C15 aryl, or a pharmaceutically acceptable salt thereof. In some embodiments, each R⁸ is CH₂NH₂ or CH₂CH₂NH₂, or a pharmaceutically acceptable salt thereof. In some embodiments, each R⁸ is naphthyl, or a pharmaceutically acceptable salt thereof.

In some embodiments, each R⁶, R⁷, N², and C² together form a 5- or 8-membered ring comprising C4-C5 heterocycloalkyl comprising a nitrogen atom and additional 0 or 1 heteroatom independently selected from N, O, and S, or a pharmaceutically acceptable salt thereof.

In some embodiments, R¹⁷ is H. In some embodiments, R⁷ is methyl. In some embodiments, R¹ is benzyl or CH₂CH(CH₃)₂.

In some embodiments, each of R¹ and R′¹ is, independently, benzyl or CH₂CH(CH₃)₂. In some embodiments, R¹ and R′¹ are each benzyl. In some embodiments, R¹ and R′¹ are each CH₂CH(CH₃)₂.

In some embodiments of this aspect, when n is 2, E dimerizes to form an Fc domain.

In some embodiments, each L′ or L comprises one or more optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, optionally substituted C2-C15 heteroarylene, O, S, NRⁱ, P, carbonyl, thiocarbonyl, sulfonyl, phosphate, phosphoryl, or imino; and Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C3-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl; or a pharmaceutically acceptable salt thereof.

In some embodiments, the backbone of each L′ or L consists of one or more optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, optionally substituted C2-C15 heteroarylene, O, S, NRⁱ, P, carbonyl, thiocarbonyl, sulfonyl, phosphate, phosphoryl, or imino; and Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C3-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl; or a pharmaceutically acceptable salt thereof.

In some embodiments, each L′ or L is oxo substituted, or a pharmaceutically acceptable salt thereof. In some embodiments, the backbone of each L′ or L comprises no more than 120 atoms, or a pharmaceutically acceptable salt thereof. In some embodiments, each L′ or L is capable of forming an amide, a carbamate, a sulfonyl, or a urea linkage, or a pharmaceutically acceptable salt thereof. In some embodiments, each L is a bond, or a pharmaceutically acceptable salt thereof.

In some embodiments, each L is described by formula (M-L-1):

J¹-(Q¹)_{g}-(T¹)ₕ-(Q²)ᵢ-(T²)ⱼ-(Q³)ₖ-(T³)ₗ-(Q⁴)ₘ-(T⁴)ₙ-(Q⁵)ₒ-J²

wherein: J¹ is a bond attached to A or M if A is absent; J² is a bond attached to E; each of Q¹, Q², Q³, Q⁴, and Q⁵ is, independently, optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene; each of T¹, T², T³, T⁴ is, independently, O, S, NRⁱ, P, carbonyl, thiocarbonyl, sulfonyl, phosphate, phosphoryl, or imino; Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C3-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl; and each of g, h, i, j, k, l, m, n, and o is, independently, 0 or 1; or a pharmaceutically acceptable salt thereof.

In some embodiments, depending on the structure and chemical formula of L-I, J² may have two points of attachment to the Fc domain (e.g., two J²).

In some embodiments, L is

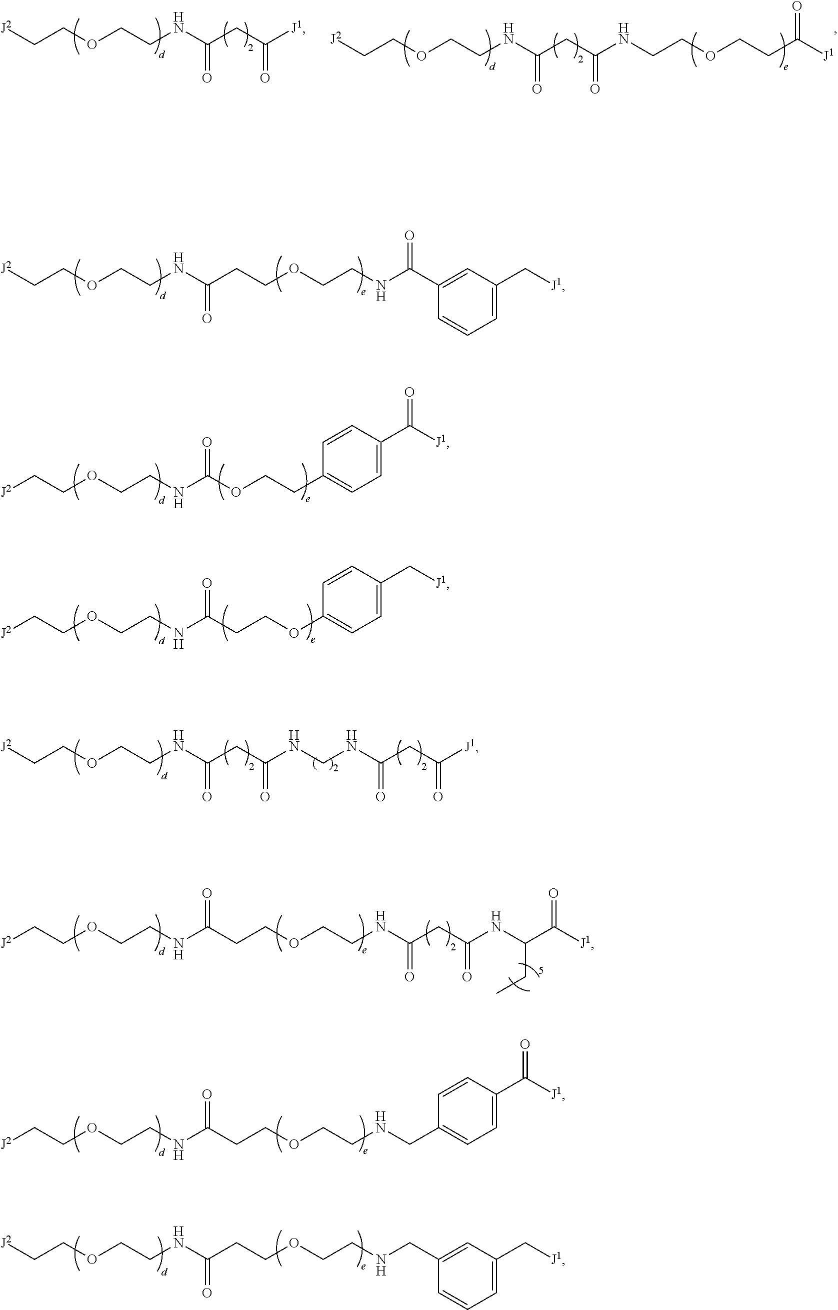

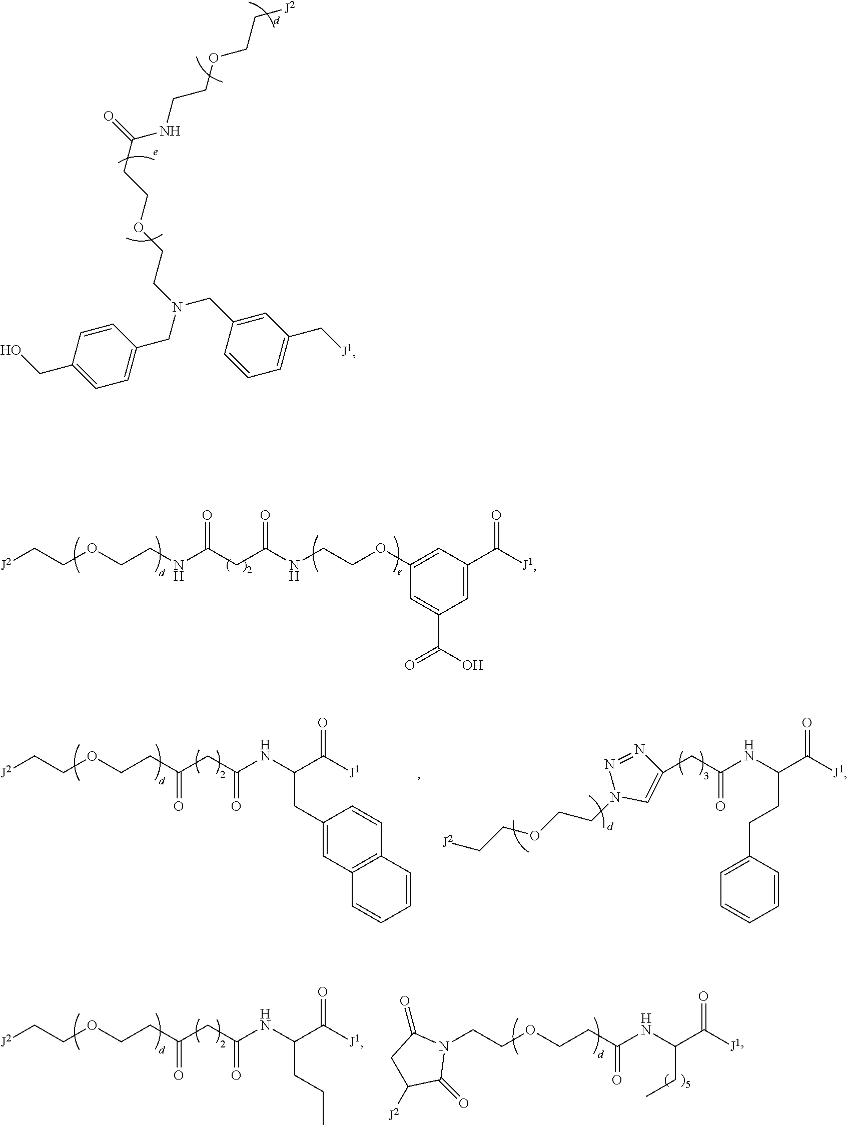

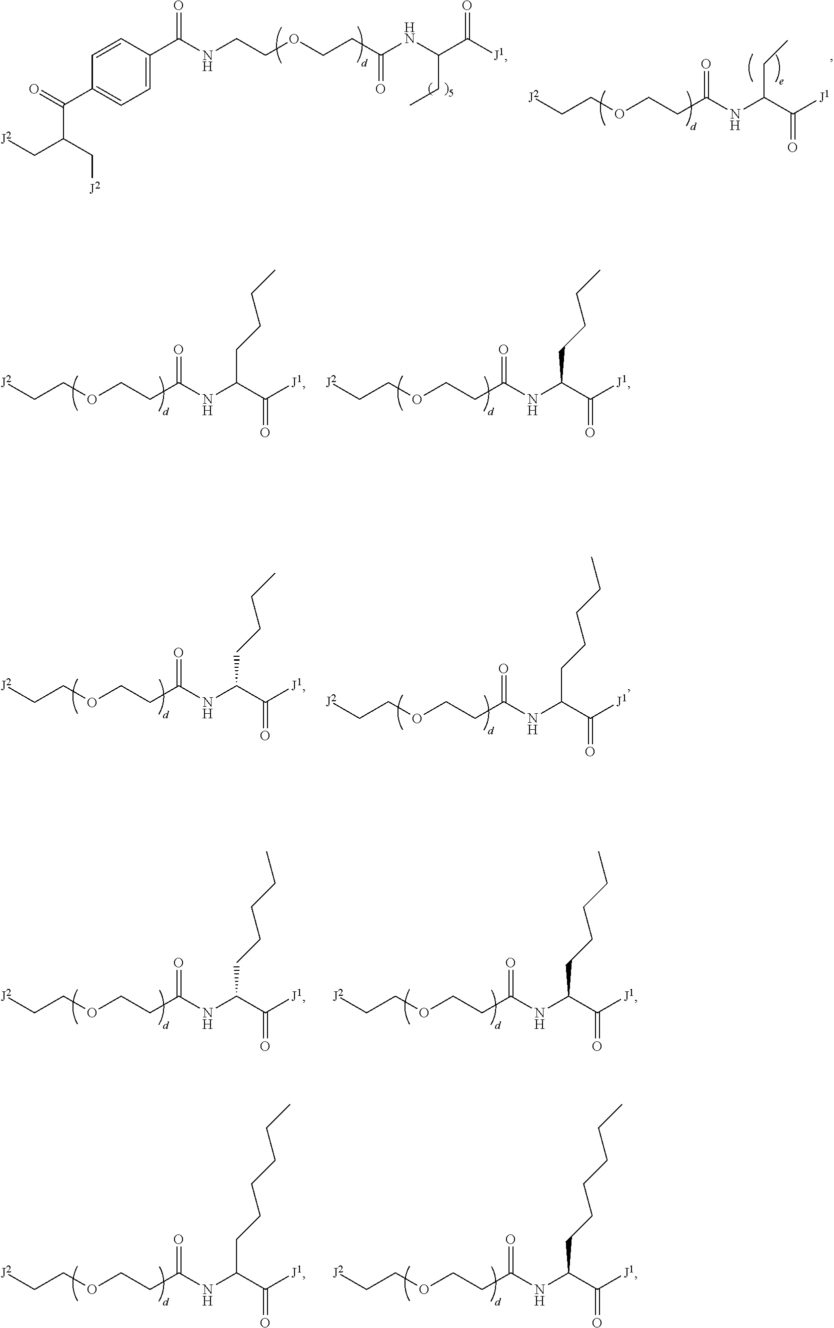

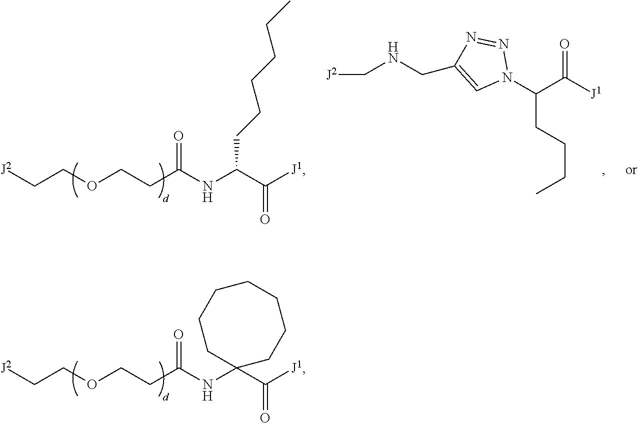

wherein each of d and e is, independently, an integer from 1 to 26; or a pharmaceutically acceptable salt thereof.

In another aspect, the invention features a conjugate of formula M-XIX):

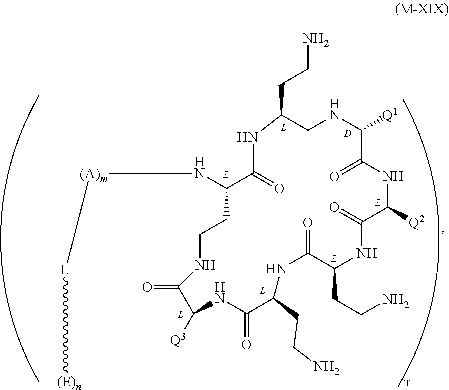

wherein:each A is an independently selected amino acid; each L is a linker that, when each m is 2, 3, 4, or 5, is bound to any of A; each E is an Fc domain monomer; each m is 0, 1, 2, 3, 4, or 5; n is 1 or 2; Tis an integer from 1 to 20, e.g., from 1 to 15; and Q¹, Q², and Q³ are each independently selected from the side chain of an amino acid; or a pharmaceutically acceptable salt thereof.

In some embodiments of this aspect, Q¹, Q², and Q³ are each independently selected from the side chain of a natural amino acid, or a pharmaceutically acceptable salt thereof.

In some embodiments, at least one of Q¹, Q², and Q³ is selected from the side chain of a non-natural amino acid, or a pharmaceutically acceptable salt thereof. In some embodiments, at least two of Q¹, Q², and Q³ are independently selected from the side chain of a non-natural amino acid, or a pharmaceutically acceptable salt thereof. In some embodiments, each of Q¹, Q², and Q³ is independently selected from the side chain of a non-natural amino acid, or a pharmaceutically acceptable salt thereof.

In some embodiments, each of Q¹, Q², and Q³ is independently selected from the side chain of serine, threonine, cysteine, glycine, proline, alanine, valine, isoleucine, leucine, methionine, phenylalanine, tyrosine, and tryptophan, or a pharmaceutically acceptable salt thereof.

In some embodiments, each of Q¹, Q², and Q³ is independently selected from C1-C4 alkyl, C1-C2 hydroxyalkyl, C1-C5 alkamino, and C6-C35 alkaryl, or a pharmaceutically acceptable salt thereof.

In some embodiments, each of Q¹, Q², and Q³ is independently selected from 2-methyl-1-propyl, 2-propyl, 1-hydroxyethyl, butyl, benzyl, hydroxymethyl, propyl, 2-butyl, methyl, and 2-aminoethyl, or a pharmaceutically acceptable salt thereof.

In some embodiments, the combination of Q¹, Q², and Q³ is selected from:

| Q¹ | Q² | Q³ |
|---|---|---|
| 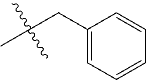 [SMILES: *Cc1ccccc1] | 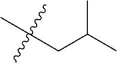 [SMILES: *CC(C)C] | 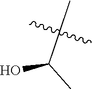 [SMILES: *[C@@H](C)O] |
| 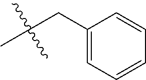 [SMILES: *Cc1ccccc1] | 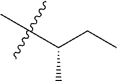 [SMILES: *[C@@H](C)CC] | 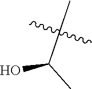 [SMILES: *[C@@H](C)O] |
| 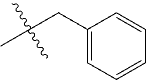 [SMILES: *Cc1ccccc1] | 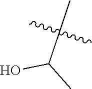 [SMILES: *C(C)O] | 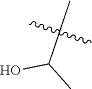 [SMILES: *C(C)O] |
| 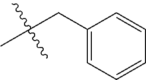 [SMILES: *Cc1ccccc1] | 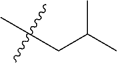 [SMILES: *CC(C)C] | 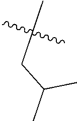 [SMILES: *CC(C)C] |
| 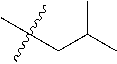 [SMILES: *CC(C)C] | 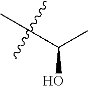 [SMILES: *[C@@H](C)O] | 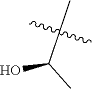 [SMILES: *[C@@H](C)O] |
| 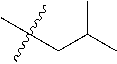 [SMILES: *CC(C)C] | 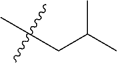 [SMILES: *CC(C)C] | 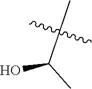 [SMILES: *[C@@H](C)O] |
| 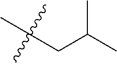 [SMILES: *CC(C)C] | 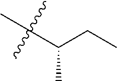 [SMILES: *[C@@H](C)CC] | 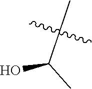 [SMILES: *[C@@H](C)O] |
| 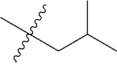 [SMILES: *CC(C)C] | 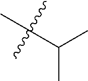 [SMILES: *C(C)C] | 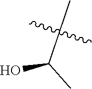 [SMILES: *[C@@H](C)O] |
| 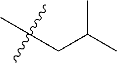 [SMILES: *CC(C)C] | 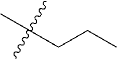 [SMILES: *CCC] | 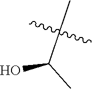 [SMILES: *[C@@H](C)O] |
| 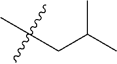 [SMILES: *CC(C)C] | —CH₃ | 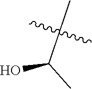 [SMILES: *[C@@H](C)O] |
| 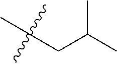 [SMILES: *CC(C)C] | —CH₂OH | 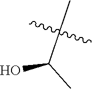 [SMILES: *[C@@H](C)O] |
| 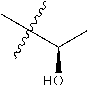 [SMILES: *[C@@H](C)O] | 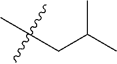 [SMILES: *CC(C)C] | 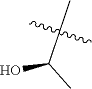 [SMILES: *[C@@H](C)O] |
| 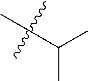 [SMILES: *C(C)C] | 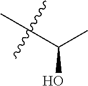 [SMILES: *[C@@H](C)O] | 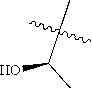 [SMILES: *[C@@H](C)O] |
| 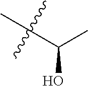 [SMILES: *[C@@H](C)O] | 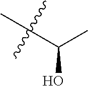 [SMILES: *[C@@H](C)O] | 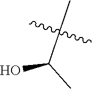 [SMILES: *[C@@H](C)O] |
| 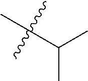 [SMILES: *C(C)C] | 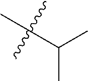 [SMILES: *C(C)C] | 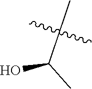 [SMILES: *[C@@H](C)O] |
| 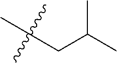 [SMILES: *CC(C)C] | —CH₂CH₂NH₂ | 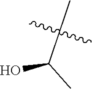 [SMILES: *[C@@H](C)O] |

or a pharmaceutically acceptable salt thereof.

In some embodiments, each m is 1, 2 or 3, or a pharmaceutically acceptable salt thereof. In some embodiments, each m is 1, 2, 3, or 4; or a pharmaceutically acceptable salt thereof.

In some embodiments, each A is independently selected from glycine, 3-aminoalanine, piperazine-2-carboxylic acid, 2,4-diaminobutyric acid, 3-hydroxyproline, threonine and 2-amino-4-phenylbutyric acid; and each m is 1, 2, 3, 4, or 5; or a pharmaceutically acceptable salt thereof.

In some embodiments, each m is 1, 2, or 3, or a pharmaceutically acceptable salt thereof.

In some embodiments, each A is independently selected from glycine, 3-aminoalanine, piperazine-2-carboxylic acid, 2,4-diaminobutyric acid, 3-hydroxyproline, threonine and 2-amino-4-phenylbutyric acid; and each m is 1, 2, 3, 4, or 5; or a pharmaceutically acceptable salt thereof.

In some embodiments, each m is 1, 2, or 3; or a pharmaceutically acceptable salt thereof.

In some embodiments of this aspect, when n is 2, E dimerizes to form an Fc domain, or a pharmaceutically acceptable salt thereof.

In another aspect, the invention features a conjugate of formula (M-XX):

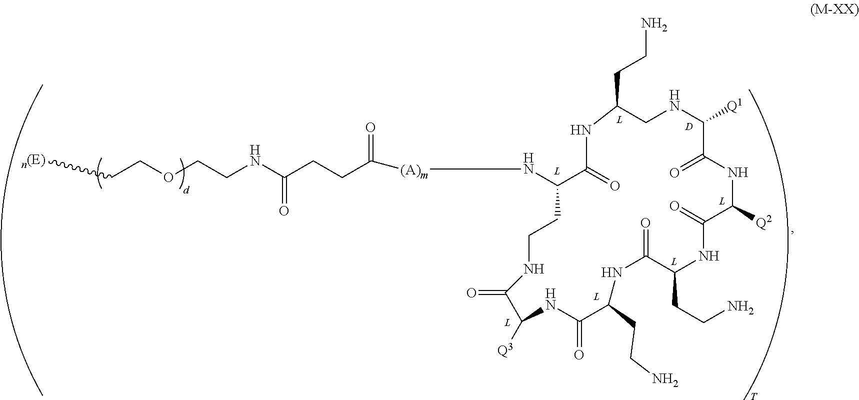

wherein: each A is an independently selected amino acid; each E is an Fc domain monomer; each m is 0, 1, 2, 3, 4, or 5; n is 1 or 2; Q¹, Q², and Q³ are each independently selected from the side chain of an amino acid; each d is 0 to 20; and T is an integer from 1 to 20, e.g., from 1 to 15; or a pharmaceutically acceptable salt thereof.

In some embodiments of this aspect, each m is 1, 2 or 3, or a pharmaceutically acceptable salt thereof. In some embodiments, each m is 1, 2, 3, or 4; or a pharmaceutically acceptable salt thereof.

In some embodiments, each A is independently selected from glycine, 3-aminoalanine, piperazine-2-carboxylic acid, 2,4-diaminobutyric acid, 3-hydroxyproline, threonine, 3-(2-naphthyl)alanine, and 2-amino-4-phenylbutyric acid; and each m is 1, 2, 3, 4, or 5; or a pharmaceutically acceptable salt thereof.

In some embodiments, each m is 1, 2, or 3, or a pharmaceutically acceptable salt thereof.

In some embodiments, each A is independently selected from glycine, 3-aminoalanine, piperazine-2-carboxylic acid, 2,4-diaminobutyic acid, 3-hydroxyproline, threonine, 3-(2-naphthyl)alanine, and 2-amino-4-phenylbutyric acid; each m is 1, 2, 3, 4, or 5; or a pharmaceutically acceptable salt thereof.

In some embodiments, each m is 1, 2, or 3; or a pharmaceutically acceptable salt thereof.

In some embodiments, each d is 0 to 10, or a pharmaceutically acceptable salt thereof. In some embodiments, each d is 0 to 5, or a pharmaceutically acceptable salt thereof. In some embodiments, each d is 0 or 1, or a pharmaceutically acceptable salt thereof.

In some embodiments, each A is independently selected from glycine, 3-aminoalanine, piperazine-2-carboxylic acid, 2,4-diaminobutyric acid, 3-hydroxyproline, threonine, 3-(2-naphthyl)alanine, and 2-amino-4-phenylbutyric acid, and 2-aminopentanoic acid; each m is 1, 2, 3, or 4: Q¹, Q², and Q³ are each independently selected from 2-methyl-1-propyl, 2-propyl, 1-hydroxyethyl, butyl, benzyl, hydroxymethyl, propyl, 2-butyl, methyl, and 2,4-diaminobutyric acid; and each d is 0 to 10; or a pharmaceutically acceptable salt thereof.

In some embodiments, Q¹, Q², and Q³ are each independently selected from 2-methyl-1-propyl, benzyl, and 1-hydroxyethyl, or a pharmaceutically acceptable salt thereof.

In some embodiments, each A is independently selected from glycine, 2,4-diaminobutyic acid, 3-hydroxyproline, 3-(2-naphthyl)alanine, and threonine, and 2-aminopentanoic acid; each m is 2 to 4; each d is 0 or 1; or a pharmaceutically acceptable salt thereof.

In another aspect, the invention features a conjugate of formula (M-XXI):

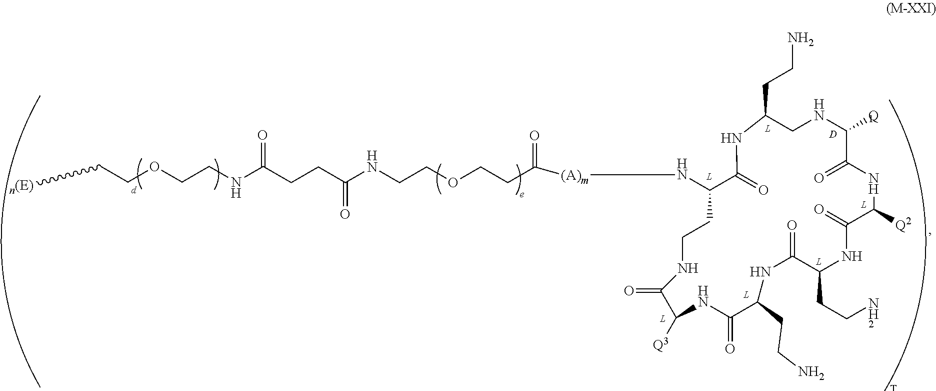

wherein: each A is an independently selected amino acid; each E is an Fc domain monomer; each m is 0, 1, 2, 3, 4, or 5; n is 1 or 2; Q1, Q2, and Q3 are each independently selected from the side chain of an amino acid; each d is an integer from 1 to 26; each e is an integer from 1 to 26; and T is an integer from 1 to 20, e.g., from 1 to 15; or a pharmaceutically acceptable salt thereof.

In some embodiments of this aspect, each m is 1, 2 or 3, or a pharmaceutically acceptable salt thereof. In some embodiments, each m is 1, 2, 3, or 4; or a pharmaceutically acceptable salt thereof.

In some embodiments, each A is independently selected from glycine, 3-aminoalanine, piperazine-2-carboxylic acid, 2,4-diaminobutyric acid, 3-hydroxyproline, threonine and 2-amino-4-phenylbutyric acid; and each m is 1, 2, 3, 4, or 5; or a pharmaceutically acceptable salt thereof.

In some embodiments, each m is 1, 2, or 3, or a pharmaceutically acceptable salt thereof.

In some embodiments, each A is independently selected from glycine, 3-aminoalanine, piperazine-2-carboxylic acid, 2,4-diaminobutyic acid, 3-hydroxyproline, threonine and 2-amino-4-phenylbutyric acid; and each m is 1, 2, 3, 4, or 5; or a pharmaceutically acceptable salt thereof.

In some embodiments, each m is 1, 2, or 3; or a pharmaceutically acceptable salt thereof.

In some embodiments, each d is an integer from 1 to 10, and each e is an integer from 1 to 10, or a pharmaceutically acceptable salt thereof.

In some embodiments, each d is an integer from 1 to 5, and each e is an integer from 1 to 6, or a pharmaceutically acceptable salt thereof. In some embodiments, each d is 1, and each e is an integer from 1 to 6, or a pharmaceutically acceptable salt thereof. In some embodiments, each d is 1, and each e is 6, or a pharmaceutically acceptable salt thereof.

In some embodiments of this aspect, each A is independently selected from glycine, 3-aminoalanine, piperazine-2-carboxylic acid, 2,4-diaminobutyric acid, 3-hydroxyproline, threonine and 2-amino-4-phenylbutyric acid; each m is 1, 2 or 3; Q¹, Q², and Q³ are each independently selected from 2-methyl-1-propyl, 2-propyl, 1-hydroxyethyl, butyl, benzyl, hydroxymethyl, propyl, 2-butyl, methyl, and 2,4-diaminobutyric acid; each d is an integer from 1 to 10; and each e is an integer from 1 to 10; or a pharmaceutically acceptable salt thereof.

In some embodiments, Q¹, Q², and Q³ are each independently selected from 2-methyl-1-propyl and 1-hydroxyethyl, or a pharmaceutically acceptable salt thereof.

In some embodiments, each A is independently selected from 2,4-diaminobutyric acid and threonine; each m is 2; each d is 1; and each e is 6; or a pharmaceutically acceptable salt thereof.

In another aspect, the invention features a conjugate of formula (M-XXII):

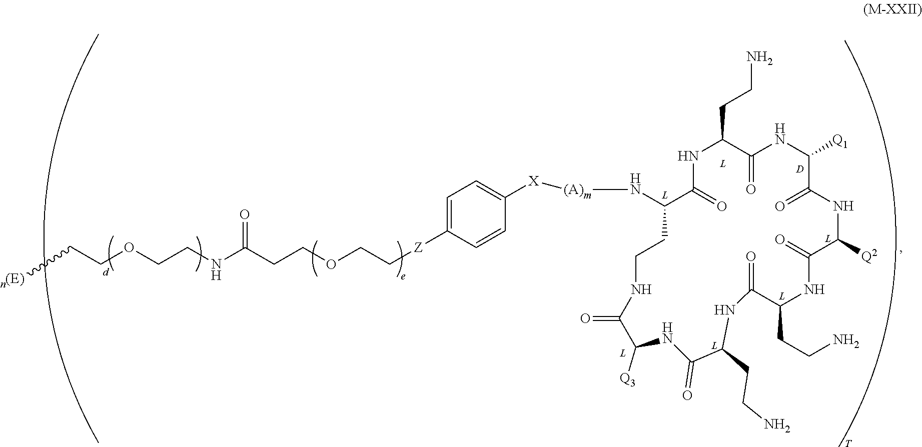

wherein: each A is an independently selected amino acid; each X is —CH₂— or —C(O)—; each Z is —C(O)NH—, —NHC(O)—, —CH₂NH—, or O; each E is an Fc domain monomer; each m is 0, 1, 2, 3, 4, or 5; n is 1 or 2; Q1, Q2, and Q3 are each independently selected from the side chain of an amino acid; each d is an integer from 1 to 26; each e is an integer from 1 to 26; and T is an integer from 1 to 20, e.g., from 1 to 15; or a pharmaceutically acceptable salt thereof.

In some embodiments, each m is 1, 2 or 3, or a pharmaceutically acceptable salt thereof. In some embodiments, each m is 1, 2, 3, or 4; or a pharmaceutically acceptable salt thereof.

In some embodiments, each A is independently selected from glycine, 2-aminoalanine, 3-aminoalanine, piperazine-2-carboxylic acid, 2,4-diaminobutyric acid, 3-hydroxyproline, threonine and 2-amino-4-phenylbutyric acid; and each m is 1, 2, 3, 4, or 5; or a pharmaceutically acceptable salt thereof.

In some embodiments, each m is 1, 2, or 3, or a pharmaceutically acceptable salt thereof.

In some embodiments, each A is independently selected from glycine, 2-aminoalanine, 3-aminoalanine, piperazine-2-carboxylic acid, 2,4-diaminobutyric acid, 3-hydroxyproline, threonine and 2-amino-4-phenylbutyric acid; each m is 1, 2, 3, 4, or 5; or a pharmaceutically acceptable salt thereof.

In some embodiments, each m is 1, 2, or 3; or a pharmaceutically acceptable salt thereof.

In some embodiments, each d is 1, and each e is 6 to 24, or a pharmaceutically acceptable salt thereof.

In some embodiments, each A is independently selected from glycine, 2-aminoalanine, 3-aminoalanine, piperazine-2-carboxylic acid, 2,4-diaminobutyric acid, 3-hydroxyproline, threonine and 2-amino-4-phenylbutyric acid; each m is 1, 2 or 3; Q¹, Q², and Q³ are each independently selected from 2-methyl-1-propyl, 2-propyl, 1-hydroxyethyl, butyl, benzyl, hydroxymethyl, propyl, 2-butyl, methyl, and 2,4-diaminobutyric acid; each d is an integer from 1 to 10; and each e is an integer from 1 to 10; or a pharmaceutically acceptable salt thereof.

In some embodiments, Q¹, Q², and Q³ are each independently selected from benzyl, 2-methyl-1-propyl and 1-hydroxyethyl, or a pharmaceutically acceptable salt thereof.

In some embodiments, each A is independently selected from 2-aminoalanine, 2,4-diaminobutyric acid, 3-hydroxyproline, piperazine-2-carboxylic acid, and threonine; each Z is, —CH₂NH—, —C(O)NH— or —NHC(O)—; each m is 2 or 3; each d is 1; and each e is 4 to 26; or a pharmaceutically acceptable salt thereof.

In some embodiments, each A is independently selected from glycine, 2-aminoalanine, 2,4-diaminobutyric acid, 3-hydroxyproline, and threonine; each Z is —CH₂NH—, or O; each m is 2; each d is 1; and each e is 4 to 26; or a pharmaceutically acceptable salt thereof.

In another aspect, a conjugate of formula (M-XXIV):

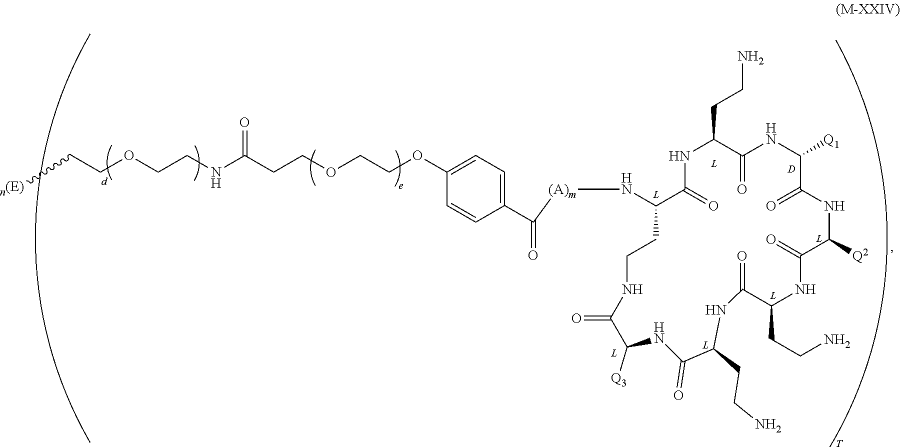

wherein: each A is an independently selected amino acid; each E is an Fc domain monomer; each m is 0, 1, 2, 3, 4, or 5; n is 1 or 2; Q1, Q2, and Q3 are each independently selected from the side chain of an amino acid; and each d is an integer from 1 to 26; each e is an integer from 1 to 26; and T is an integer from 1 to 20, e.g., from 1 to 15; or a pharmaceutically acceptable salt thereof.

In some embodiments, each m is 1, 2 or 3, or a pharmaceutically acceptable salt thereof. In some embodiments, each m is 1, 2, 3, or 4; or a pharmaceutically acceptable salt thereof.

In some embodiments, each A is independently selected from glycine, 3-aminoalanine, piperazine-2-carboxylic acid, 2,4-diaminobutyic acid, 3-hydroxyproline, threonine and 2-amino-4-phenylbutyric acid; and each m is 1, 2, 3, 4, or 5; or a pharmaceutically acceptable salt thereof.

In some embodiments, each m is 1, 2, or 3, or a pharmaceutically acceptable salt thereof.

In some embodiments, each A is independently selected from glycine, 3-aminoalanine, piperazine-2-carboxylic acid, 2,4-diaminobutyric acid, 3-hydroxyproline, threonine and 2-amino-4-phenylbutyric acid; each m is 1, 2, or 3; or a pharmaceutically acceptable salt thereof.

In some embodiments, each d is an integer from 1 to 10, and each e is an integer from 1 to 10, or a pharmaceutically acceptable salt thereof. In some embodiments, each d is an integer from 1 to 5, and each e is an integer from 1 to 5, or a pharmaceutically acceptable salt thereof. In some embodiments, each d is 1, and each e is 4, or a pharmaceutically acceptable salt thereof.

In some embodiments, each A is independently selected from glycine, 3-aminoalanine, piperazine-2-carboxylic acid, 2,4-diaminobutyric acid, 3-hydroxyproline, threonine and 2-amino-4-phenylbutyric acid; each m is 1, 2 or 3; Q¹, Q², and Q³ are each independently selected from 2-methyl-1-propyl, 2-propyl, 1-hydroxyethyl, butyl, benzyl, hydroxymethyl, propyl, 2-butyl, methyl, and 2,4-diaminobutyric acid; each d is an integer from 1 to 10; and each e is an integer from 1 to 10; or a pharmaceutically acceptable salt thereof.

In some embodiments, Q¹, Q², and Q³ are each independently selected from benzyl, 2-methyl-1-propyl and 1-hydroxyethyl, or a pharmaceutically acceptable salt thereof.

In some embodiments, each A is independently selected from 2,4-diaminobutyric acid and threonine; each m is 2; each d is 1; and each e is 4; or a pharmaceutically acceptable salt thereof.

In another aspect, the invention features a conjugate of formula (M-XXV):

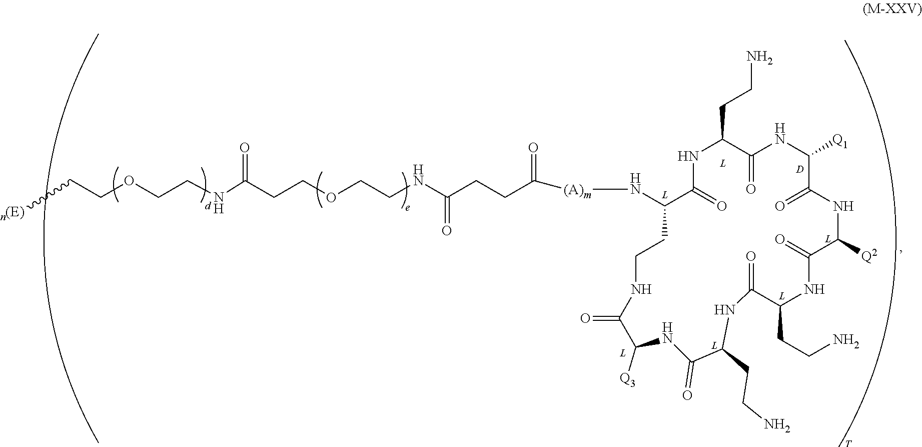

wherein: each A is an independently selected amino acid; each E is an Fc domain monomer; each m is 0, 1, 2, 3, 4, or 5; n is 1 or 2; Q1, Q2, and Q3 are each independently selected from the side chain of an amino acid; each d is an integer from 1 to 26; each e is an integer from 1 to 26; T is an integer from 1 to 20, e.g., from 1 to 15; or a pharmaceutically acceptable salt thereof.

In some embodiments, each m is 1, 2 or 3, or a pharmaceutically acceptable salt thereof. In some embodiments, each m is 1, 2, 3, or 4; or a pharmaceutically acceptable salt thereof.

In some embodiments, each A is independently selected from glycine, 3-aminoalanine, piperazine-2-carboxylic acid, 2,4-diaminobutyic acid, 3-hydroxyproline, threonine, 2-aminooctanoic acid, and 2-amino-4-phenylbutyric acid; and each m is 1, 2, 3, 4, or 5; or a pharmaceutically acceptable salt thereof.

In some embodiments, each m is 1, 2, or 3, or a pharmaceutically acceptable salt thereof.

In some embodiments, each A is independently selected from glycine, 3-aminoalanine, piperazine-2-carboxylic acid, 2,4-diaminobutyic acid, 3-hydroxyproline, threonine, 2-aminooctanoic acid, and 2-amino-4-phenylbutyric acid; each m is 1, 2, 3, or 4; or a pharmaceutically acceptable salt thereof.

In some embodiments, each d is an integer from 1 to 10, and each e is an integer from 1 to 10, or a pharmaceutically acceptable salt thereof. In some embodiments, each d is an integer from 1 to 5, and each e is an integer from 1 to 5, or a pharmaceutically acceptable salt thereof.

In some embodiments, each m is 4, each d is 1, and each e is 4, or a pharmaceutically acceptable salt thereof.

In some embodiments, each A is independently selected from glycine, 3-aminoalanine, piperazine-2-carboxylic acid, 2,4-diaminobutyric acid, 3-hydroxyproline, threonine, 2-aminooctanoic acid, and 2-amino-4-phenylbutyric acid; each m is 1, 2, 3, or 4; Q¹, Q², and Q³ are each independently selected from 2-methyl-1-propyl, 2-propyl, 1-hydroxyethyl, butyl, benzyl, hydroxymethyl, propyl, 2-butyl, methyl, and 2,4-diaminobutyric acid; each d is 1; and each e is an integer from 1 to 5; or a pharmaceutically acceptable salt thereof.

In some embodiments, Q¹, Q², and Q³ are each independently selected from benzyl, 2-methyl-1-propyl and 1-hydroxyethyl, or a pharmaceutically acceptable salt thereof.

In some embodiments, each A is independently selected from 2,4-diaminobutyric acid, 2-aminooctanoic acid, and threonine; each m is 4; each d is 1; and each e is 4; or a pharmaceutically acceptable salt thereof.

In another aspect, the invention features a conjugate of formula (M-XXVI):

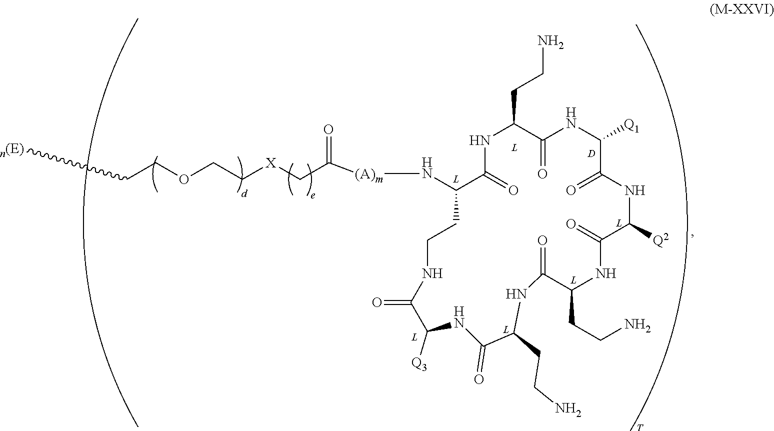

wherein: each A is an independently selected amino acid; each X is heteroaryl or heterocyclyl; each E is an Fc domain monomer; each m is 0, 1, 2, 3, 4, or 5; n is 1 or 2; Q1, Q2, and Q3 are each independently selected from the side chain of an amino acid; each d is an integer from 1 to 26; each e is an integer from 1 to 26; and T is an integer from 1 to 20, e.g., from 1 to 15; or a pharmaceutically acceptable salt thereof.

In some embodiments, each m is 1, 2 or 3, or a pharmaceutically acceptable salt thereof. In some embodiments, each m is 1, 2, 3, or 4; or a pharmaceutically acceptable salt thereof.

In some embodiments, each A is independently selected from glycine, 3-aminoalanine, piperazine-2-carboxylic acid, 2,4-diaminobutyric acid, 3-hydroxyproline, threonine, 2-aminooctanoic acid, 3-(2-naphthyl)alanine, and 2-amino-4-phenylbutyric acid; and each m is 1, 2, 3, 4, or 5; or a pharmaceutically acceptable salt thereof.

In some embodiments, each m is 1, 2, 3, or 4, or a pharmaceutically acceptable salt thereof.

In some embodiments, each A is independently selected from glycine, 3-aminoalanine, piperazine-2-carboxylic acid, 2,4-diaminobutyric acid, 3-hydroxyproline, threonine, 2-aminooctanoic acid, 3-(2-naphthyl)alanine, and 2-amino-4-phenylbutyric acid; each m is 1, 2, 3, or 4; or a pharmaceutically acceptable salt thereof.

In some embodiments, each d is an integer from 1 to 10, and each e is an integer from 1 to 10, or a pharmaceutically acceptable salt thereof. In some embodiments, each d is an integer from 1 to 5, and each e is an integer from 1 to 5, or a pharmaceutically acceptable salt thereof. In some embodiments, each m is 4, each d is 1, and each e is 4, or a pharmaceutically acceptable salt thereof.

In some embodiments, each A is independently selected from glycine, 3-aminoalanine, piperazine-2-carboxylic acid, 2,4-diaminobutyic acid, 3-hydroxyproline, threonine, 2-aminooctanoic acid, 3-(2-naphthyl)alanine, and 2-amino-4-phenylbutyric acid; each X is heteroaryl; each m is 1, 2, 3, or 4; Q¹, Q², and Q³ are each independently selected from 2-methyl-1-propyl, 2-propyl, 1-hydroxyethyl, butyl, benzyl, hydroxymethyl, propyl, 2-butyl, methyl, and 2,4-diaminobutyric acid; each d is an integer from 1 to 5; and each e is an integer from 1 to 5; or a pharmaceutically acceptable salt thereof.

In some embodiments, each A is independently selected from 2,4-diaminobutyric acid, threonine, 2-aminooctanoic acid, and 2-amino-4-phenylbutyric acid; each m is 2, 3, or 4; and each e is an integer from 1 to 3; or a pharmaceutically acceptable salt thereof.

In some embodiments, each X is triazolyl, or a pharmaceutically acceptable salt thereof. In some embodiments, each X is 1,4-triazolyl, or a pharmaceutically acceptable salt thereof. In some embodiments, Q¹, Q², and Q³ are each independently selected from benzyl, 2-methyl-1-propyl and 1-hydroxyethyl, or a pharmaceutically acceptable salt thereof.

In some embodiments, each A is independently selected from 2,4-diaminobutyric acid, 2-aminooctanoic acid, and threonine; each m is 4; each d is 1; and each e is 3; or a pharmaceutically acceptable salt thereof.

In some embodiments of the conjugates described herein, Q¹, Q², and Q³ are each independently selected from the side chain of a natural amino acid, or a pharmaceutically acceptable salt thereof. In some embodiments, at least one of Q¹, Q², and Q³ is selected from the side chain of a non-natural amino acid, or a pharmaceutically acceptable salt thereof. In some embodiments, at least two of Q¹, Q², and Q³ are independently selected from the side chain of a non-natural amino acid, or a pharmaceutically acceptable salt thereof. In some embodiments, each of Q¹, Q², and Q³ is independently selected from the side chain of a non-natural amino acid, or a pharmaceutically acceptable salt thereof. In some embodiments, each of Q¹, Q², and Q³ is independently selected from the side chain of serine, threonine, cysteine, glycine, proline, alanine, valine, isoleucine, leucine, methionine, phenylalanine, tyrosine, and tryptophan, or a pharmaceutically acceptable salt thereof. In some embodiments, each of Q¹, Q², and Q³ is independently selected from C1-C4 alkyl, C1-C2 hydroxyalkyl, C1-C5 alkamino, and C6-C35 alkaryl, or a pharmaceutically acceptable salt thereof.

In some embodiments, each of Q¹, Q², and Q³ is independently selected from 2-methyl-1-propyl, 2-propyl, 1-hydroxyethyl, butyl, benzyl, hydroxymethyl, propyl, 2-butyl, methyl, and 2-aminoethyl, or a pharmaceutically acceptable salt thereof.

In some embodiments, the combination of Q¹, Q², and Q³ is selected from:

| Q¹ | Q² | Q³ |
|---|---|---|
| 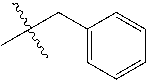 [SMILES: *Cc1ccccc1] | 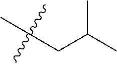 [SMILES: *CC(C)C] | 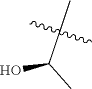 [SMILES: *[C@@H](C)O] |
| 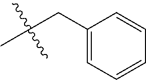 [SMILES: *Cc1ccccc1] | 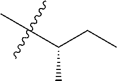 [SMILES: *[C@@H](C)CC] | 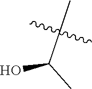 [SMILES: *[C@@H](C)O] |
| 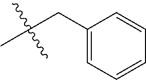 [SMILES: *Cc1ccccc1] | 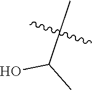 [SMILES: *C(C)O] | 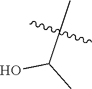 [SMILES: *C(C)O] |
| 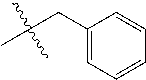 [SMILES: *Cc1ccccc1] | 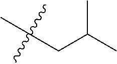 [SMILES: *CC(C)C] | 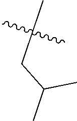 [SMILES: *CC(C)C] |
| 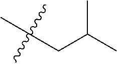 [SMILES: *CC(C)C] | 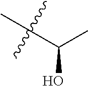 [SMILES: *[C@@H](C)O] | 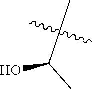 [SMILES: *[C@@H](C)O] |
| 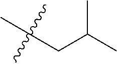 [SMILES: *CC(C)C] | 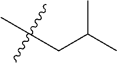 [SMILES: *CC(C)C] | 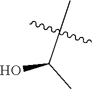 [SMILES: *[C@@H](C)O] |
| 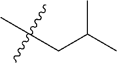 [SMILES: *CC(C)C] | 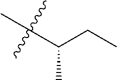 [SMILES: *[C@@H](C)CC] | 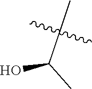 [SMILES: *[C@@H](C)O] |
| 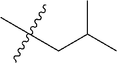 [SMILES: *CC(C)C] | 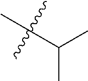 [SMILES: *C(C)C] | 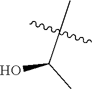 [SMILES: *[C@@H](C)O] |
| 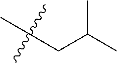 [SMILES: *CC(C)C] | 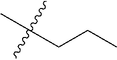 [SMILES: *CCC] | 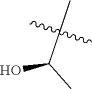 [SMILES: *[C@@H](C)O] |
| 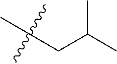 [SMILES: *CC(C)C] | —CH₃ | 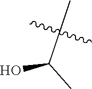 [SMILES: *[C@@H](C)O] |
| 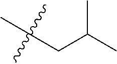 [SMILES: *CC(C)C] | —CH₂OH | 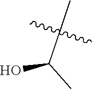 [SMILES: *[C@@H](C)O] |
| 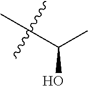 [SMILES: *[C@@H](C)O] | 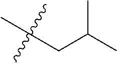 [SMILES: *CC(C)C] | 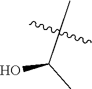 [SMILES: *[C@@H](C)O] |
| 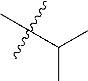 [SMILES: *C(C)C] | 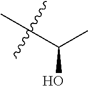 [SMILES: *[C@@H](C)O] | 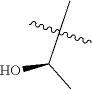 [SMILES: *[C@@H](C)O] |
| 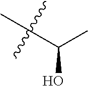 [SMILES: *[C@@H](C)O] | 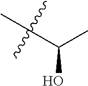 [SMILES: *[C@@H](C)O] | 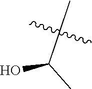 [SMILES: *[C@@H](C)O] |
| 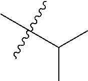 [SMILES: *C(C)C] | 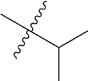 [SMILES: *C(C)C] | 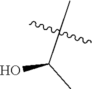 [SMILES: *[C@@H](C)O] |
| 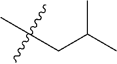 [SMILES: *CC(C)C] | —CH₂CH₂NH₂ | 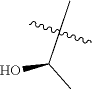 [SMILES: *[C@@H](C)O] |

or a pharmaceutically acceptable salt thereof.

In some embodiments of the conjugates described herein, when n is 2, E dimerizes to form an Fc domain, or a pharmaceutically acceptable salt thereof.

In some embodiments of any of the aspects of a conjugate described herein, a concentration of the conjugate, or a pharmaceutically acceptable salt thereof, that activates an immune cell is less than or equal to 10,000 nM. In some embodiments, a concentration of the conjugate, or a pharmaceutically acceptable salt thereof, that activates an immune cell is less than or equal to equal to 1,000 nM. In some embodiments, a concentration of the conjugate, or a pharmaceutically acceptable salt thereof, that activates an immune cell is less than or equal to equal to 100 nM.

In another aspect, the invention features a pharmaceutical composition including a conjugate described herein, or a pharmaceutically acceptable salt thereof, and a pharmaceutically acceptable excipient. In some embodiments, the pharmaceutical composition further includes an antibacterial agent.

In some embodiments, the antibacterial agent is selected from the group consisting of linezolid, tedizolid, posizolid, radezolid, retapamulin, valnemulin, tiamulin, azamulin, lefamulin, plazomicin, amikacin, gentamicin, gamithromycin, kanamycin, neomycin, netilmicin, tobramycin, paromomycin, streptomycin, spectinomycin, geldanamycin, herbimycin, rifaximin, loracarbef, ertapenem, doripenem, imipenem/cilastatin, meropenem, cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil, ceftobiprole, teicoplanin, vancomycin, telavancin, dalbavancin, oritavancin, clindamycin, lincomycin, daptomycin, solithromycin, azithromycin, clarithromycin, dirithromycin, erythromycin, roxithromycin, troleandomycin, telithromycin, spiramycin, aztreonam, furazolidone, nitrofurantoin, amoxicillin, ampicillin, azlocillin, carbenicillin, cloxacillin, dicloxacillin, flucloxacillin, mezlocillin, methicillin, nafcillin, oxacillin, penicillin g, penicillin v, piperacillin, penicillin g, temocillin, ticarcillin, amoxicillin clavulanate, ampicillin/sulbactam, piperacillin/tazobactam, ticarcillin/clavulanate, bacitracin, ciprofloxacin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfacetamide, sulfadiazine, silver sulfadiazine, sulfadimethoxine, sulfamethizole, sulfamethoxazole, sulfanilimide, sulfasalazine, sulfisoxazole, trimethoprim-sulfamethoxazole (tmp-smx), sulfonamidochrysoidine, eravacycline, demeclocycline, doxycycline, minocycline, oxytetracycline, tetracycline, clofazimine, dapsone, capreomycin, cycloserine, ethambutol (bs), ethionamide, isoniazid, pyrazinamide, rifampicin, rifabutin, rifapentine, streptomycin, arsphenamine, chloramphenicol, fosfomycin, fusidic acid, metronidazole, mupirocin, platensimycin, quinupristin/dalfopristin, thiamphenicol, tigecycline, tinidazole, and trimethoprim, prodrugs thereof, and pharmaceutically acceptable salts thereof. In some embodiments, a prodrug of tedizolid is tedizolid phosphate.

In some embodiments, the antibacterial agent is tedizolid, azithromycin, meropenem, amikacin, levofloxacin, rifampicin, linezolid, erythromycin, or solithromycin. In some embodiments, the antibacterial agent is tedizolid, azithromycin, meropenem, amikacin, or levofloxacin.

In another aspect, the invention features a method of protecting against or treating a bacterial infection in a subject, the method including administering to the subject a conjugate described herein. In some embodiments, the method further includes administering to the subject an antibacterial agent.

In another aspect, the invention features a method of protecting against or treating a bacterial infection in a subject, the method including administering to the subject (1) a conjugate described herein and (2) an antibacterial agent.

In another aspect, the invention features a method of inducing immune cell activation of the immune response in a subject having a bacterial infection, the method including administering to the subject a conjugate described herein. In some embodiments, the method further includes administering to the subject an antibacterial agent.

In another aspect, the invention features a method of inducing immune cell activation of the immune response in a subject having a bacterial infection, the method including administering to the subject (1) a conjugate described herein and (2) an antibacterial agent.

In some embodiments of the methods, the conjugate and the antibacterial agent are administered substantially simultaneously. In some embodiments, the conjugate and the antibacterial agent are administered separately. In some embodiments, the conjugate is administered first, followed by administering of the antibacterial agent alone. In some embodiments, the antibacterial agent is administered first, followed by administering of the conjugate alone. In some embodiments, the conjugate and the antibacterial agent are administered substantially simultaneously, followed by administering of the conjugate or the antibacterial agent alone. In some embodiments, the conjugate or the antibacterial agent is administered first, followed by administering of the conjugate and the antibacterial agent substantially simultaneously.

In some embodiments of the methods, administering the conjugate and the antibacterial agent together lowers the MIC of each of the conjugate and the antibacterial agent relative to the MIC of each of the conjugate and the antibacterial agent when each is used alone.

In some embodiments of the methods, the conjugate and/or the antibacterial agent is administered intramuscularly, intravenously, intradermally, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctival, intravesicularlly, mucosally, intrapericardially, intraumbilically, intraocularally, orally, locally, by inhalation, by injection, or by infusion.

In another aspect, the invention features a method of preventing, stabilizing, or inhibiting the growth of bacteria, or killing bacteria, including contacting the bacteria or a site susceptible to bacterial growth with a conjugate described herein.

In some embodiments, the method further includes contacting the bacteria or the site susceptible to bacterial growth with an antibacterial agent.

In another aspect, the invention features a method of preventing, stabilizing, or inhibiting the growth of bacteria, or killing bacteria, including contacting the bacteria or a site susceptible to bacterial growth with (1) a conjugate described herein and (2) an antibacterial agent.

In some embodiments of the methods described herein, the antibacterial agent is selected from the group consisting of linezolid, tedizolid, posizolid, radezolid, retapamulin, valnemulin, tiamulin, azamulin, lefamulin, plazomicin, amikacin, gentamicin, gamithromycin, kanamycin, neomycin, netilmicin, tobramycin, paromomycin, streptomycin, spectinomycin, geldanamycin, herbimycin, rifaximin, loracarbef, ertapenem, doripenem, imipenem/cilastatin, meropenem, cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil, ceftobiprole, teicoplanin, vancomycin, telavancin, dalbavancin, oritavancin, clindamycin, lincomycin, daptomycin, solithromycin, azithromycin, clarithromycin, dirithromycin, erythromycin, roxithromycin, troleandomycin, telithromycin, spiramycin, aztreonam, furazolidone, nitrofurantoin, amoxicillin, ampicillin, azlocillin, carbenicillin, cloxacillin, dicloxacillin, flucloxacillin, mezlocillin, methicillin, nafcillin, oxacillin, penicillin g, penicillin v, piperacillin, penicillin g, temocillin, ticarcillin, amoxicillin clavulanate, ampicillin/sulbactam, piperacillin/tazobactam, ticarcillin/clavulanate, bacitracin, ciprofloxacin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfacetamide, sulfadiazine, silver sulfadiazine, sulfadimethoxine, sulfamethizole, sulfamethoxazole, sulfanilimide, sulfasalazine, sulfisoxazole, trimethoprim-sulfamethoxazole (tmp-smx), sulfonamidochrysoidine, eravacycline, demeclocycline, doxycycline, minocycline, oxytetracycline, tetracycline, clofazimine, dapsone, capreomycin, cycloserine, ethambutol(bs), ethionamide, isoniazid, pyrazinamide, rifampicin, rifabutin, rifapentine, streptomycin, arsphenamine, chloramphenicol, fosfomycin, fusidic acid, metronidazole, mupirocin, platensimycin, quinupristin/dalfopristin, thiamphenicol, tigecycline, tinidazole, and trimethoprim, prodrugs thereof, and pharmaceutically acceptable salts thereof. In some embodiments, a prodrug of tedizolid is tedizolid phosphate.

In some embodiments of the methods, the antibacterial agent is tedizolid, azithromycin, meropenem, amikacin, levofloxacin, rifampicin, linezolid, erythromycin, or solithromycin. In some embodiments of the methods, the antibacterial agent is tedizolid, azithromycin, meropenem, amikacin, or levofloxacin.

In some embodiments of the methods, the bacterial infection is caused by Gram-negative bacteria. In some embodiments, the bacterial infection is caused by a resistant strain of bacteria. In some embodiments, the resistant strain of bacteria possesses the mcr-1 gene, the mcr-2 gene, the mcr-3 gene, the mcr-4 gene, the mcr-5 gene, the mcr-6 gene, the mcr-7 gene, the mcr-8 gene, and/or a chromosomal mutation conferring polymyxin resistance. In some embodiments, the resistant strain of bacteria possesses the mcr-1 gene. In some embodiments, the resistant strain of bacteria possesses the mcr-2 gene. In some embodiments, the resistant strain of bacteria possesses the mcr-3 gene. In some embodiments, the resistant strain of bacteria possesses the mcr-4 gene. In some embodiments, the resistant strain of bacteria possesses the mcr-5 gene. In some embodiments, the resistant strain of bacteria possesses the mcr-6 gene. In some embodiments, the resistant strain of bacteria possesses the mcr-7 gene. In some embodiments, the resistant strain of bacteria possesses the mcr-8 gene. In some embodiments, the resistant strain of bacteria possesses a chromosomal mutation conferring polymyxin resistance. In some embodiments, the resistant strain of bacteria is a resistant strain of E. coli.

In another aspect, the invention features a method of preventing lipopolysaccharides (LPS) in Gram-negative bacteria from activating an immune system in a subject, including administering to the subject a conjugate described herein. In some embodiments, the method prevents LPS from activating a macrophage. In some embodiments, the method prevents LPS-induced nitric oxide (NO) production from a macrophage.

### Definitions

The term “cyclic heptapeptide” or “cycloheptapeptide,” as used herein, refers to certain compounds that bind to lipopolysaccharides (LPS) in the cell membrane of Gram-negative bacteria to disrupt and permeabilize the cell membrane, leading to cell death and/or sensitization to other antibiotics. Cyclic heptapeptides or cycloheptapeptides comprise seven natural or non-natural α-amino acid residues, such as D- or L-amino acid residues, in a closed ring. Generally, cyclic heptapeptides are formed by linking the α-carboxyl group of one amino acid to the α-amino group or the γ-amino group of another amino acid and cyclizing. The cyclic heptapeptide comprises a heterocycle comprising carbon and nitrogen ring members, which may be substituted, for example, with amino acid side chains. One nitrogen from an α-amino group in the cyclic heptapeptide, however, is not a ring member and is branched from a ring member of the heterocycle. Thus, this nitrogen is directly attached to a ring member, such as a carbon atom (e.g., an α-carbon atom). This nitrogen atom serves as an attachment point for the cyclic heptapeptide to a linker and/or to a peptide (e.g., a peptide including 1-5 amino acid residue(s)), and thus is referred to herein as a “linking nitrogen.” The linking nitrogen is directly attached to the ring of the cyclic heptapeptide and is not derived from a side chain, such as an ethylamine side chain. The linking nitrogens in a conjugate of, e.g., formula (II) or (III), are N⁴ and N′⁴.

In some embodiments, a peptide including one or more (e.g., 1-5; 1, 2, 3, 4, or 5) amino acid residues (e.g., natural and/or non-natural amino acid residues) may be covalently attached to a linking nitrogen (e.g., N⁴ and/or N′⁴, the nitrogen from an α-amino group) in the cyclic heptapeptide ring. Cyclic heptapeptides may be derived from polymyxins (e.g., naturally existing polymyxins and non-natural polymyxins) and/or octapeptins (e.g., naturally existing octapeptins and non-natural octapeptins). Examples of naturally existing polymyxins include, but are not limited to, polymyxin B₁, polymyxin B₂, polymyxin B₃, polymyxin B₄, polymyxin B₅, polymyxin B₆, polymyxin B₁-Ile, polymyxin B₂-Ile, polymyxin C₁, polymyxin C₂, polymyxin S₁, polymyxin T₁, polymyxin T2, polymyxin A₁, polymyxin A₂, polymyxin D₁, polymyxin D₂, polymyxin E₁ (colistin A), polymyxin E₂ (colistin B), polymyxin E₃, polymyxin E₄, polymyxin E₇, polymyxin E₁-Ile, polymyxin E₁-Val, polymyxin E₁-Nva, polymyxin E₂-Ile, polymyxin E₂-Val, polymyxin E₂-Nva, polymyxin E₈-Ile, polymyxin M₁, and polymyxin M₂. In other embodiments, a cyclic heptapeptide may be entirely synthetic and prepared by standard peptide methodology as known in the art.

As used herein, the term “Fc domain monomer” refers to a polypeptide chain that includes at least a hinge domain and second and third antibody constant domains (C_{H}2 and C_{H}3) or functional fragments thereof (e.g., fragments that that capable of (i) dimerizing with another Fc domain monomer to form an Fc domain, and (ii) binding to an Fc receptor. The Fc domain monomer can be any immunoglobulin antibody isotype, including IgG, IgE, IgM, IgA, or IgD (e.g., IgG). Additionally, the Fc domain monomer can be an IgG subtype (e.g., IgG1, IgG2a, IgG2b. IgG3, or IgG4) (e.g., IgG1). An Fc domain monomer does not include any portion of an immunoglobulin that is capable of acting as an antigen-recognition region, e.g., a variable domain or a complementarity determining region (CDR). Fc domain monomers in the conjugates as described herein can contain one or more changes from a wild-type Fc domain monomer sequence (e.g., 1-10, 1-8, 1-6, 1-4 amino acid substitutions, additions, or deletions) that alter the interaction between an Fc domain and an Fc receptor. Examples of suitable changes are known in the art.

As used herein, the term “Fc domain” refers to a dimer of two Fc domain monomers that is capable of binding an Fc receptor. In the wild-type Fc domain, the two Fc domain monomers dimerize by the interaction between the two C_{H}3 antibody constant domains, in some embodiments, one or more disulfide bonds form between the hinge domains of the two dimerizing Fc domain monomers.

The term “covalently attached” refers to two parts of a conjugate that are linked to each other by a covalent bond formed between two atoms in the two parts of the conjugate. For example, in the conjugate described herein (e.g., a conjugate of any one of formulas (I)-(XXXVI)), when a′ is 0, L is covalently attached to N′⁴, which means that when a′ is 0, an atom in L forms a covalent bond with N′⁴ in the conjugate. Similarly, when a′ is 1 and b′ is 0, L is covalently attached to N′⁴; when b′ is 1, and c′ is 0, L is covalently attached to N′²; when c′ is 1, L is covalently attached to N′³; when a is 0, L is covalently attached to N⁴; when a is 1 and b is 0, L is covalently attached to N¹; when b is 1, and c is 0, L is covalently attached to N²; and when c is 1, L is covalently attached to N³.

The terms “linker,” “L′,” “L,” and “L′,” as used herein, refer to a covalent linkage or connection between two or more components in a conjugate (e.g., between two cyclic heptapeptides in a conjugate described herein, between a cyclic heptapeptide and an Fc domain in a conjugate described herein, and between a dimer of two cyclic peptapeptide and an Fc domain in a conjugate described herein). In some embodiments, a conjugate described herein may contain a linker that has a trivalent structure (e.g., a trivalent linker). A trivalent linker has three arms, in which each arm is covalently linked to a component of the conjugate (e.g., a first arm conjugated to a first cyclic heptapeptide, a second arm conjugated to a second cyclic heptapeptide, and a third arm conjugated to an Fc domain).

Molecules that may be used as linkers include at least two functional groups, which may be the same or different, e.g., two carboxylic acid groups, two amine groups, two sulfonic acid groups, a carboxylic acid group and a maleimide group, a carboxylic acid group and an alkyne group, a carboxylic acid group and an amine group, a carboxylic acid group and a sulfonic acid group, an amine group and a maleimide group, an amine group and an alkyne group, or an amine group and a sulfonic acid group. The first functional group may form a covalent linkage with a first component in the conjugate and the second functional group may form a covalent linkage with the second component in the conjugate. In some embodiments of a trivalent linker, two arms of a linker may contain two dicarboxylic acids, in which the first carboxylic acid may form a covalent linkage with the first cyclic heptapeptide in the conjugate and the second carboxylic acid may form a covalent linkage with the second cyclic heptapeptide in the conjugate, and the third arm of the linker may for a covalent linkage with an Fc domain in the conjugate. Examples of dicarboxylic acids are described further herein. In some embodiments, a molecule containing one or more maleimide groups may be used as a linker, in which the maleimide group may form a carbon-sulfur linkage with a cysteine in a component (e.g., an Fc domain) in the conjugate. In some embodiments, a molecule containing one or more alkyne groups may be used as a linker, in which the alkyne group may form a 1,2,3-triazole linkage with an azide in a component (e.g., an Fc domain) in the conjugate. In some embodiments, a molecule containing one or more azide groups may be used as a linker, in which the azide group may form a 1,2,3-triazole linkage with an alkyne in a component (e.g., an Fc domain) in the conjugate. In some embodiments, a molecule containing one or more bis-sulfone groups may be used as a linker, in which the bis-sulfone group may form a linkage with an amine group a component (e.g., an Fc domain) in the conjugate. In some embodiments, a molecule containing one or more sulfonic acid groups may be used as a linker, in which the sulfonic acid group may form a sulfonamide linkage with a component in the conjugate. In some embodiments, a molecule containing one or more isocyanate groups may be used as a linker, in which the isocyanate group may form a urea linkage with a component in the conjugate. In some embodiments, a molecule containing one or more haloalkyl groups may be used as a linker, in which the haloalkyl group may form a covalent linkage, e.g., C—N and C—O linkages, with a component in the conjugate.

In some embodiments, a linker provides space, rigidity, and/or flexibility between the two or more components. In some embodiments, a linker may be a bond, e.g., a covalent bond. The term “bond” refers to a chemical bond, e.g., an amide bond, a disulfide bond, a C—O bond, a C—N bond, a N—N bond, a C—S bond, or any kind of bond created from a chemical reaction, e.g., chemical conjugation. In some embodiments, a linker includes no more than 250 atoms. In some embodiments, a linker includes no more than 250 non-hydrogen atoms. In some embodiments, the backbone of a linker includes no more than 250 atoms. The “backbone” of a linker refers to the atoms in the linker that together form the shortest path from one part of a conjugate to another part of the conjugate (e.g., the shortest path linking a first cyclic heptapeptide and a second cyclic heptapeptide). The atoms in the backbone of the linker are directly involved in linking one part of a conjugate to another part of the conjugate (e.g., linking a first cyclic heptapeptide and a second cyclic heptapeptide). For examples, hydrogen atoms attached to carbons in the backbone of the linker are not considered as directly involved in linking one part of the conjugate to another part of the conjugate.

In some embodiments, a linker may comprise a synthetic group derived from, e.g., a synthetic polymer (e.g., a polyethylene glycol (PEG) polymer). In some embodiments, a linker may comprise one or more amino acid residues, such as D- or L-amino acid residues. In some embodiments, a linker may be a residue of an amino acid sequence (e.g., a 1-25 amino acid, 1-10 amino acid, 1-9 amino acid, 1-8 amino acid, 1-7 amino acid, 1-6 amino acid, 1-5 amino acid, 1-4 amino acid, 1-3 amino acid, 1-2 amino acid, or 1 amino acid sequence). In some embodiments, a linker may comprise one or more, e.g., 1-100, 1-50, 1-25, 1-10, 1-5, or 1-3, optionally substituted alkylene, optionally substituted heteroalkylene (e.g., a PEG unit), optionally substituted alkenylene, optionally substituted heteroalkenylene, optionally substituted alkynylene, optionally substituted heteroalkynylene, optionally substituted cycloalkylene, optionally substituted heterocycloalkylene, optionally substituted cycloalkenylene, optionally substituted heterocycloalkenylene, optionally substituted cycloalkynylene, optionally substituted heterocycloalkynylene, optionally substituted arylene, optionally substituted heteroarylene (e.g., pyridine), O, S, NRⁱ (Rⁱ is H, optionally substituted alkyl, optionally substituted heteroalkyl, optionally substituted alkenyl, optionally substituted heteroalkenyl, optionally substituted alkynyl, optionally substituted heteroalkynyl, optionally substituted cycloalkyl, optionally substituted heterocycloalkyl, optionally substituted cycloalkenyl, optionally substituted heterocycloalkenyl, optionally substituted cycloalkynyl, optionally substituted heterocycloalkynyl, optionally substituted aryl, or optionally substituted heteroaryl), P, carbonyl, thiocarbonyl, sulfonyl, phosphate, phosphoryl, or imino. For example, a linker may comprise one or more optionally substituted C1-C20 alkylene, optionally substituted C1-C20 heteroalkylene (e.g., a PEG unit), optionally substituted C2-C20 alkenylene (e.g., C2 alkenylene), optionally substituted C2-C20 heteroalkenylene, optionally substituted C2-C20 alkynylene, optionally substituted C2-C20 heteroalkynylene, optionally substituted C3-C20 cycloalkylene (e.g., cyclopropylene, cyclobutylene), optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene (e.g., C6 arylene), optionally substituted C2-C15 heteroarylene (e.g., imidazole, pyridine), O, S, NRⁱ (Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C3-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl), P, carbonyl, thiocarbonyl, sulfonyl, phosphate, phosphoryl, or imino.

The term “lipophilic moiety,” as used herein, refers to a portion, substituent, or functional group of a compound that is, in general, hydrophobic and non-polar. A moiety is lipophilic if it has a hydrophobicity determined using a cLogP value of greater than 0, such as about 0.25 or greater, about 0.5 or greater, about 1 or greater, about 2 or greater, 0.25-5, 0.5-4 or 2-3. As used herein, the term “cLogP” refers to the calculated partition coefficient of a molecule or portion of a molecule. The partition coefficient is the ratio of concentrations of a compound in a mixture of two immiscible phases at equilibrium (e.g., octanol and water) and measures the hydrophobicity or hydrophilicity of a compound. A variety of methods are available in the art for determining cLogP. For example, in some embodiments, cLogP can be determined using quantitative structure-property relationship algorithms known in the art (e.g., using fragment based prediction methods that predict the log P of a compound by determining the sum of its non-overlapping molecular fragments). Several algorithms for calculating cLogP are known in the art including those used by molecular editing software such as CHEMDRAW® Pro, Version 12.0.2.1092 (Camrbridgesoft, Cambridge, Mass.) and MARVINSKETCH® (ChemAxon, Budapest, Hungary). A moiety is considered lipophilic if it has a cLogP value described above in at least one of the above methods. A lipophilic moiety having the stated cLogP value will be considered lipophilic, even though it may have a positive charge or a polar substituent.

In some embodiments, a lipophilic moiety contains entirely hydrocarbons. In some embodiments, a lipophilic moiety may contain one or more, e.g., 1-4, 1-3, 1, 2, 3, or 4, heteroatoms independently selected from N, O, and S (e.g., an indolyl), or one or more, e.g., 1-4, 1-3, 1, 2, 3, or 4, halo groups, which, due to the structure of the moiety and/or small differences in electronegativity between the heteroatoms or halo groups and the hydrocarbons, do not induce significant chemical polarity into the lipophilic moiety. Thus, in some embodiments, a lipophilic moiety having, e.g., 1-4, 1-3, 1, 2, 3, or 4, heteroatoms and/or, e.g., 1-4, 1-3, 1, 2, 3, or 4, halo atoms may still be considered non-polar. In some embodiments, a lipophilic moiety may be optionally substituted alkyl, optionally substituted alkenyl, optionally substituted alkynyl, optionally substituted aryl, optionally substituted heteroalkyl, optionally substituted heteroalkenyl, optionally substituted heteroalkynyl, or optionally substituted heteroaryl, or halo forms thereof, wherein the optional substituents are also lipophilic (such as alkyl, alkenyl, alkynyl, heteroalkyl, heteroalkenyl, heteroalkynyl, aryl, or heteroaryl) or are not lipophilic but do not change the overall lipophilic character of the moiety, i.e., the moiety has a cLogP value of greater than 0. For example, octanol contains a polar group, OH, but is still a lipophilic moiety. In some embodiments, a lipophilic moiety may be benzyl, isobutyl, sec-butyl, isopropyl, n-propyl, methyl, biphenylmethyl, n-octyl, or substituted indolyl (e.g., alkyl substituted indolyl). In some embodiments, a lipophilic moiety may be the side chain of a hydrophobic amino acid residue, e.g., leucine, isoleucine, alanine, phenylalanine, valine, and proline, or groups such as methyl, ethyl, propyl, isopropyl, butyl, isobutyl, and pyrrolidinyl. In some embodiments, lipophilic moieties of the conjugates described herein may interact with the hydrophobic portions of lipid A (e.g., fatty acid side chains of lipid A) when the conjugates bind to the membrane of bacterial cells (e.g., Gram-negative bacterial cells). Due to its position on the cyclic heptapeptide, one or more of R¹, R¹², R¹⁵, R′¹, R′¹², and R¹⁵ may be a lipophilic moiety.

The term “positively charged moiety,” as used herein, refers to a portion, substituent, or functional group of a compound that contains at least one positive charge. In some embodiments, a positively charged moiety contains one or more (e.g., 1-4, 1-3, 1, 2, 3, or 4) heteroatoms independently selected from N, O, and S, for example. In some embodiments, a positively charged moiety may possess a pH-dependent positive charge, e.g., the moiety becomes a positively charged moiety at physiological pH (e.g., pH 7), such as —NH₃⁺, —(CH₂)₄NH₂, —(CH₂)₃NH₂, —(CH₂)₂NH₂, —CH₂NH₂, —(CH₂)₄N(CH₃)₂, —(CH₂)₃N(CH₃)₂, —(CH₂)₂N(CH₃)₂, —CH₂N(CH₃)₂, —(CH₂)₄NH(CH₃), —(CH₂)₃NH(CH₃), —(CH₂)₂NH(CH₃), and —CH₂NH(CH₃). In some embodiments, a positively charged moiety may be optionally substituted alkamino, optionally substituted heteroalkyl (e.g., optionally substituted heteroalkyl containing 1-3 nitrogens; —(CH₂)₄-guanidinium, —(CH₂)₃-guanidinium, —(CH₂)₂-guanidinium, —CH₂-guanidinium), optionally substituted heterocycloalkyl (e.g., optionally substituted heterocycloalkyl containing 1-3 nitrogens), or optionally substituted heteroaryl (e.g., optionally substituted heteroaryl containing 1-3 nitrogens; —(CH₂)₄-imidazole, —(CH₂)₂-imidazole, —(CH₂)₂-imidazole, —CH₂-imidazole). In some embodiments, a positively charged moiety may be pH independent such as —CH₂N(CH₃)₃⁺, —(CH₂)₂N(CH₃)₃⁺, —(CH₂)₃N(CH₃)₃⁺, or —(CH₂)₄N(CH₃)₃⁺. Thus, substituents may transform an otherwise lipophilic moiety such as optionally substituted alkyl, optionally substituted alkenyl, optionally substituted alkynyl, optionally substituted aryl, optionally substituted heteroalkyl, optionally substituted heteroalkenyl, optionally substituted heteroalkynyl, or optionally substituted heteroaryl, or halo forms thereof, to a positively charged moiety with the addition of a substituent that imparts a positive charge or a pH dependent positive charge, such as guanidinyl, —NH₃⁺, —NH₂, —NH(CH₃), —N(CH₃)₂, and/or —N(CH₃)₃⁺. In some embodiments, a positively charged moiety may be the side chain of an amino acid residue (e.g., a natural or non-natural amino acid residue, such as a D- or L-amino acid residue, that is positively charged at physiological pH (e.g., pH 7), such as the side chain of a basic amino acid residue (e.g., arginine, lysine, histidine, omithine, diaminobuteric acid, or diaminopropionic acid). In some embodiments, positively charged moieties of the conjugates described herein interact with the negatively charged portions of lipid A (e.g., phosphates of lipid A) when the conjugates bind to the membrane of bacterial cells (e.g., Gram-negative bacterial cells). Due to its position on the cyclic heptapeptide, one or more of R¹¹, R¹³, R¹⁴, R′¹¹, R′¹³, and R′¹⁴ may be a positively charged moiety.

The term “polar moiety,” as used herein, refers to a portion, substituent, or functional group of a compound that has a chemical polarity induced by atoms with different electronegativity. The polarity of a polar moiety is dependent on the electronegativity between atoms within the moiety and the asymmetry of the structure of the moiety. In some embodiments, a polar moiety contains one or more (e.g., 1-4, 1-3, 1, 2, 3, or 4) heteroatoms independently selected from N, O, and S, which may induce chemical polarity in the moiety by having different electronegativity from carbon and hydrogen. In general, a polar moiety interacts with other polar or charged molecules. In some embodiments, a polar moiety may be optionally substituted alkamino, optionally substituted heteroalkyl (e.g., N- and/or O-containing heteroalkyl; —(CH₂)₄-carboxylic acid, —(CH₂)₃-carboxylic acid, —(CH₂)₂-carboxylic acid, —CH₂-carboxylic acid), optionally substituted heterocycloalkyl (e.g., N- and/or O-containing heterocycloalkyl), or optionally substituted heteroaryl (e.g., N- and/or O-containing heteroaryl). In some embodiments, a polar moiety may —CH(CH₃)OH, —CH₂OH, —(CH₂)₂CONH₂, —CH₂CONH₂, —CH₂COOH, or —(CH₂)₂COOH. Thus, substituents may transform an otherwise lipophilic moiety such optionally substituted alkyl, optionally substituted alkenyl, optionally substituted alkynyl, optionally substituted aryl, optionally substituted heteroalkyl, optionally substituted heteroalkenyl, optionally substituted heteroalkynyl, or optionally substituted heteroaryl, or halo forms thereof, to a polar moiety with the addition of a substituent that imparts polarity, such as —OH, —COOH, —COOR, or —CONR₂, in which R is H or C1-C4 alkyl. In some embodiments, a polar moiety may be the side chain or a polar or charged amino acid residue (e.g., threonine, serine, glutamine, asparagine, arginine, lysine histidine, aspartic acid, and glutamic acid). In some embodiments, a polar moiety is the side chain of threonine. In some embodiments, polar moieties of the conjugates described herein interact with the negatively charged portions of lipid A (e.g., phosphates of lipid A) when the conjugates bind to the membrane of bacterial cells (e.g., Gram-negative bacterial cells). Due to its position on the cyclic heptapeptide, one or more of R¹, R¹², R¹⁵, R′¹, R′¹², and R′¹⁵ may be a polar moiety.

The term “polymyxin core.” as used herein means a cyclic heptapeptide having the structure:

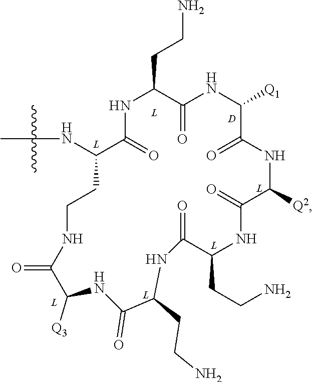

wherein Q¹, Q² and Q³ are as follows:

| Q¹ | Q² | Q³ |
|---|---|---|
| 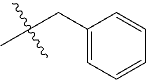 [SMILES: *Cc1ccccc1] | 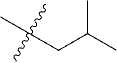 [SMILES: *CC(C)C] | 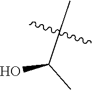 [SMILES: *[C@@H](C)O] |
| 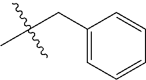 [SMILES: *Cc1ccccc1] | 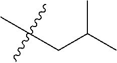 [SMILES: *CC(C)C] | 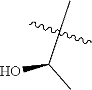 [SMILES: *[C@@H](C)O] |
| 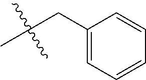 [SMILES: *Cc1ccccc1] | 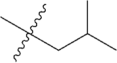 [SMILES: *CC(C)C] | 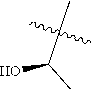 [SMILES: *[C@@H](C)O] |
| 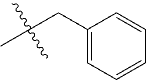 [SMILES: *Cc1ccccc1] | 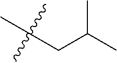 [SMILES: *CC(C)C] | 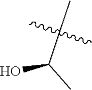 [SMILES: *[C@@H](C)O] |
| 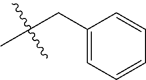 [SMILES: *Cc1ccccc1] | 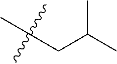 [SMILES: *CC(C)C] | 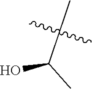 [SMILES: *[C@@H](C)O] |
| 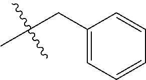 [SMILES: *Cc1ccccc1] | 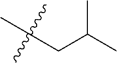 [SMILES: *CC(C)C] | 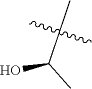 [SMILES: *[C@@H](C)O] |
| 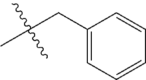 [SMILES: *Cc1ccccc1] | 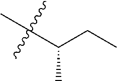 [SMILES: *[C@@H](C)CC] | 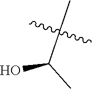 [SMILES: *[C@@H](C)O] |
| 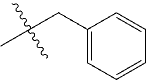 [SMILES: *Cc1ccccc1] | 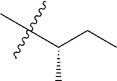 [SMILES: *[C@@H](C)CC] | 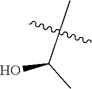 [SMILES: *[C@@H](C)O] |
| 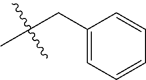 [SMILES: *Cc1ccccc1] | 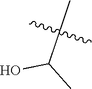 [SMILES: *C(C)O] | 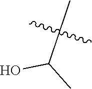 [SMILES: *C(C)O] |
| 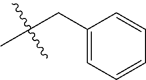 [SMILES: *Cc1ccccc1] | 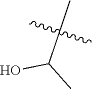 [SMILES: *C(C)O] | 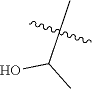 [SMILES: *C(C)O] |
| 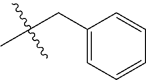 [SMILES: *Cc1ccccc1] | 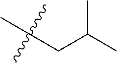 [SMILES: *CC(C)C] | 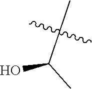 [SMILES: *[C@@H](C)O] |
| 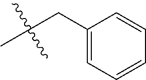 [SMILES: *Cc1ccccc1] | 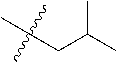 [SMILES: *CC(C)C] | 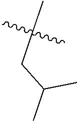 [SMILES: *CC(C)C] |
| 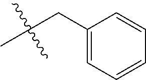 [SMILES: *Cc1ccccc1] | 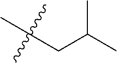 [SMILES: *CC(C)C] | 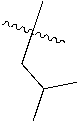 [SMILES: *CC(C)C] |
| 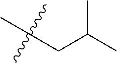 [SMILES: *CC(C)C] | 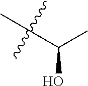 [SMILES: *[C@@H](C)O] | 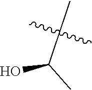 [SMILES: *[C@@H](C)O] |
| 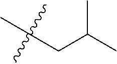 [SMILES: *CC(C)C] | 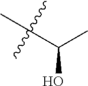 [SMILES: *[C@@H](C)O] | 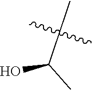 [SMILES: *[C@@H](C)O] |
| 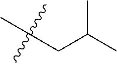 [SMILES: *CC(C)C] | 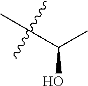 [SMILES: *[C@@H](C)O] | 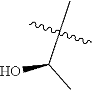 [SMILES: *[C@@H](C)O] |
| 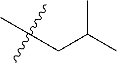 [SMILES: *CC(C)C] | 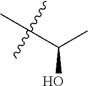 [SMILES: *[C@@H](C)O] | 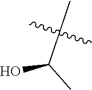 [SMILES: *[C@@H](C)O] |
| 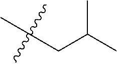 [SMILES: *CC(C)C] | 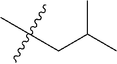 [SMILES: *CC(C)C] | 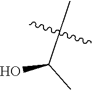 [SMILES: *[C@@H](C)O] |
| 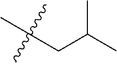 [SMILES: *CC(C)C] | 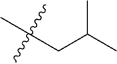 [SMILES: *CC(C)C] | 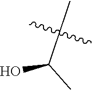 [SMILES: *[C@@H](C)O] |
| 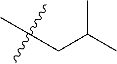 [SMILES: *CC(C)C] | 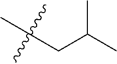 [SMILES: *CC(C)C] | 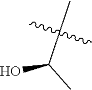 [SMILES: *[C@@H](C)O] |
| 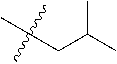 [SMILES: *CC(C)C] | 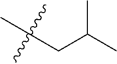 [SMILES: *CC(C)C] | 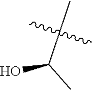 [SMILES: *[C@@H](C)O] |
| 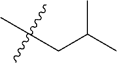 [SMILES: *CC(C)C] | 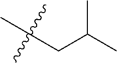 [SMILES: *CC(C)C] | 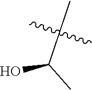 [SMILES: *[C@@H](C)O] |
| 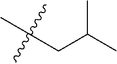 [SMILES: *CC(C)C] | 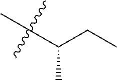 [SMILES: *[C@@H](C)CC] | 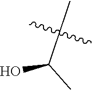 [SMILES: *[C@@H](C)O] |
| 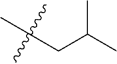 [SMILES: *CC(C)C] | 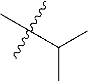 [SMILES: *C(C)C] | 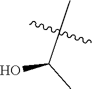 [SMILES: *[C@@H](C)O] |
| 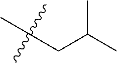 [SMILES: *CC(C)C] | 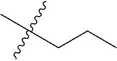 [SMILES: *CCC] | 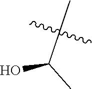 [SMILES: *[C@@H](C)O] |
| 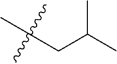 [SMILES: *CC(C)C] | 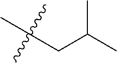 [SMILES: *CC(C)C] | 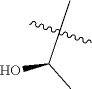 [SMILES: *[C@@H](C)O] |
| 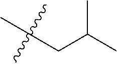 [SMILES: *CC(C)C] | 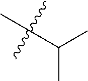 [SMILES: *C(C)C] | 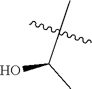 [SMILES: *[C@@H](C)O] |
| 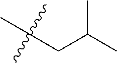 [SMILES: *CC(C)C] | 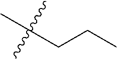 [SMILES: *CCC] | 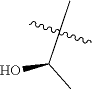 [SMILES: *[C@@H](C)O] |
| 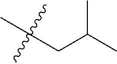 [SMILES: *CC(C)C] | 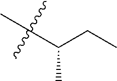 [SMILES: *[C@@H](C)CC] | 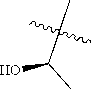 [SMILES: *[C@@H](C)O] |
| 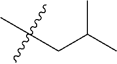 [SMILES: *CC(C)C] | 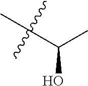 [SMILES: *[C@@H](C)O] | 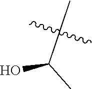 [SMILES: *[C@@H](C)O] |
| 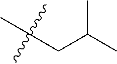 [SMILES: *CC(C)C] | 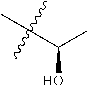 [SMILES: *[C@@H](C)O] | 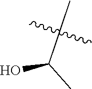 [SMILES: *[C@@H](C)O] |
| 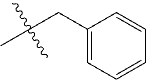 [SMILES: *Cc1ccccc1] | 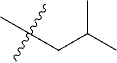 [SMILES: *CC(C)C] | 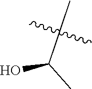 [SMILES: *[C@@H](C)O] |
| 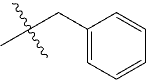 [SMILES: *Cc1ccccc1] | 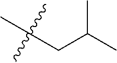 [SMILES: *CC(C)C] | 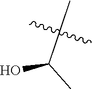 [SMILES: *[C@@H](C)O] |
| 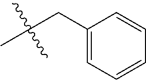 [SMILES: *Cc1ccccc1] | 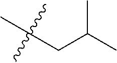 [SMILES: *CC(C)C] | 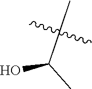 [SMILES: *[C@@H](C)O] |
| 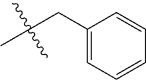 [SMILES: *Cc1ccccc1] | 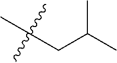 [SMILES: *CC(C)C] | 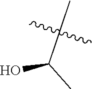 [SMILES: *[C@@H](C)O] |
| 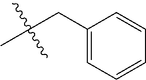 [SMILES: *Cc1ccccc1] | 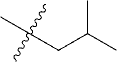 [SMILES: *CC(C)C] | 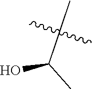 [SMILES: *[C@@H](C)O] |
| 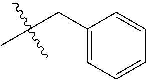 [SMILES: *Cc1ccccc1] | 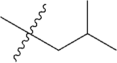 [SMILES: *CC(C)C] | 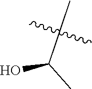 [SMILES: *[C@@H](C)O] |
| 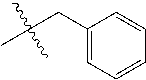 [SMILES: *Cc1ccccc1] | 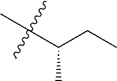 [SMILES: *[C@@H](C)CC] | 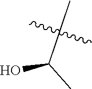 [SMILES: *[C@@H](C)O] |
| 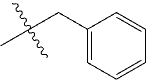 [SMILES: *Cc1ccccc1] | 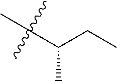 [SMILES: *[C@@H](C)CC] | 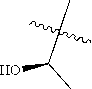 [SMILES: *[C@@H](C)O] |
| 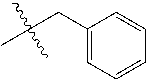 [SMILES: *Cc1ccccc1] | 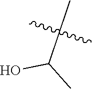 [SMILES: *C(C)O] | 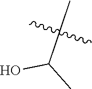 [SMILES: *C(C)O] |
| 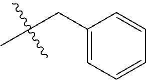 [SMILES: *Cc1ccccc1] | 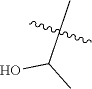 [SMILES: *C(C)O] | 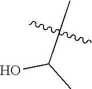 [SMILES: *C(C)O] |
| 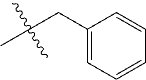 [SMILES: *Cc1ccccc1] | 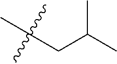 [SMILES: *CC(C)C] | 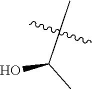 [SMILES: *[C@@H](C)O] |
| 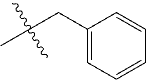 [SMILES: *Cc1ccccc1] | 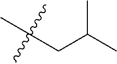 [SMILES: *CC(C)C] | 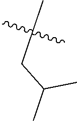 [SMILES: *CC(C)C] |
| 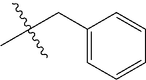 [SMILES: *Cc1ccccc1] | 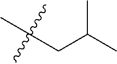 [SMILES: *CC(C)C] | 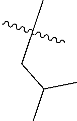 [SMILES: *CC(C)C] |
| 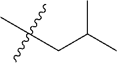 [SMILES: *CC(C)C] | 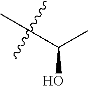 [SMILES: *[C@@H](C)O] | 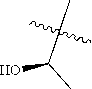 [SMILES: *[C@@H](C)O] |
| 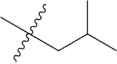 [SMILES: *CC(C)C] | 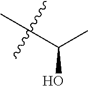 [SMILES: *[C@@H](C)O] | 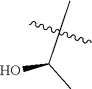 [SMILES: *[C@@H](C)O] |
| 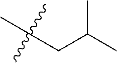 [SMILES: *CC(C)C] | 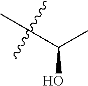 [SMILES: *[C@@H](C)O] | 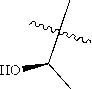 [SMILES: *[C@@H](C)O] |
| 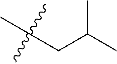 [SMILES: *CC(C)C] | 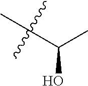 [SMILES: *[C@@H](C)O] | 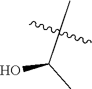 [SMILES: *[C@@H](C)O] |
| 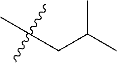 [SMILES: *CC(C)C] | 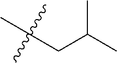 [SMILES: *CC(C)C] | 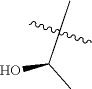 [SMILES: *[C@@H](C)O] |
| 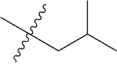 [SMILES: *CC(C)C] | 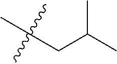 [SMILES: *CC(C)C] | 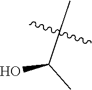 [SMILES: *[C@@H](C)O] |
| 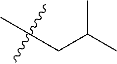 [SMILES: *CC(C)C] | 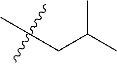 [SMILES: *CC(C)C] | 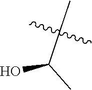 [SMILES: *[C@@H](C)O] |
| 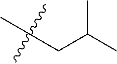 [SMILES: *CC(C)C] | 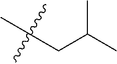 [SMILES: *CC(C)C] | 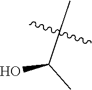 [SMILES: *[C@@H](C)O] |
| 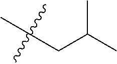 [SMILES: *CC(C)C] | 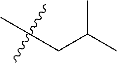 [SMILES: *CC(C)C] | 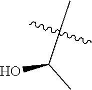 [SMILES: *[C@@H](C)O] |
| 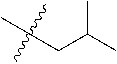 [SMILES: *CC(C)C] | 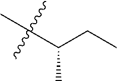 [SMILES: *[C@@H](C)CC] | 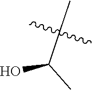 [SMILES: *[C@@H](C)O] |
| 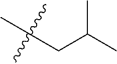 [SMILES: *CC(C)C] | 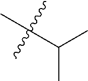 [SMILES: *C(C)C] | 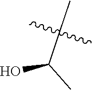 [SMILES: *[C@@H](C)O] |
| 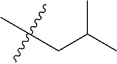 [SMILES: *CC(C)C] | 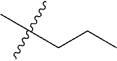 [SMILES: *CCC] | 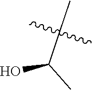 [SMILES: *[C@@H](C)O] |
| 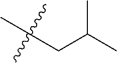 [SMILES: *CC(C)C] | 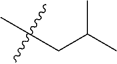 [SMILES: *CC(C)C] | 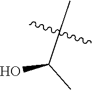 [SMILES: *[C@@H](C)O] |
| 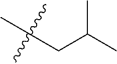 [SMILES: *CC(C)C] | 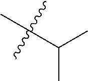 [SMILES: *C(C)C] | 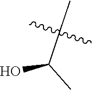 [SMILES: *[C@@H](C)O] |
| 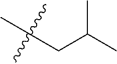 [SMILES: *CC(C)C] | 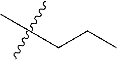 [SMILES: *CCC] | 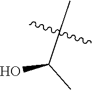 [SMILES: *[C@@H](C)O] |
| 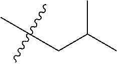 [SMILES: *CC(C)C] | 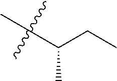 [SMILES: *[C@@H](C)CC] | 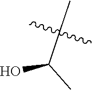 [SMILES: *[C@@H](C)O] |
| 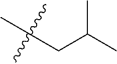 [SMILES: *CC(C)C] | 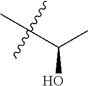 [SMILES: *[C@@H](C)O] | 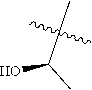 [SMILES: *[C@@H](C)O] |
| 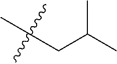 [SMILES: *CC(C)C] | 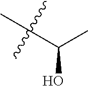 [SMILES: *[C@@H](C)O] | 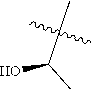 [SMILES: *[C@@H](C)O] |
| D-Leu | L-Thr | 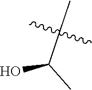 [SMILES: *[C@@H](C)O] |
| D-Leu | L-Ala | 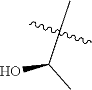 [SMILES: *[C@@H](C)O] |
| D-Leu | L-Val | 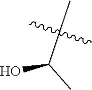 [SMILES: *[C@@H](C)O] |
| D-Nle | L-Thr | 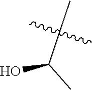 [SMILES: *[C@@H](C)O] |
| D-Leu | L-Ser | 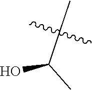 [SMILES: *[C@@H](C)O] |
| D-Thr | L-Leu | 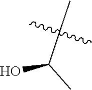 [SMILES: *[C@@H](C)O] |
| D-Val | L-Thr | 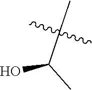 [SMILES: *[C@@H](C)O] |
| D-Thr | L-Thr | 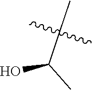 [SMILES: *[C@@H](C)O] |
| D-Val | L-Val | 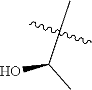 [SMILES: *[C@@H](C)O] |
| D-Leu | L-Thr | 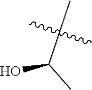 [SMILES: *[C@@H](C)O] |
| D-Leu | L-Ala | 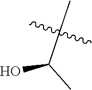 [SMILES: *[C@@H](C)O] |
| D-Leu | L-Abu | 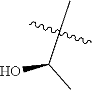 [SMILES: *[C@@H](C)O] |

wherein “D-Nle” is D-norleucine, “L-Abu” is L-2-aminobutyric acid, and “ [symbol] ” refers to the point of attachment of the polymyxin core to the remainder of the conjugates disclosed herein, including the second polymyxin core (in conjugates that contain an Fc domain covalently linked to one or more dimers of polymyxin cores), the linker, and the Fc domain of the conjugates disclosed herein.

The terms “alkyl,” “alkenyl,” and “alkynyl,” as used herein, include straight-chain and branched-chain monovalent substituents, as well as combinations of these, containing only C and H when unsubstituted. When the alkyl group includes at least one carbon-carbon double bond or carbon-carbon triple bond, the alkyl group can be referred to as an “alkenyl” or “alkynyl” group respectively. The monovalency of an alkyl, alkenyl, or alkynyl group does not include the optional substituents on the alkyl, alkenyl, or alkynyl group. For example, if an alkyl, alkenyl, or alkynyl group is attached to a compound, monovalency of the alkyl, alkenyl, or alkynyl group refers to its attachment to the compound and does not include any additional substituents that may be present on the alkyl, alkenyl, or alkynyl group. In some embodiments, the alkyl or heteroalkyl group may contain, e.g., 1-20, 1-18, 1-16, 1-14, 1-12, 1-10, 1-8, 1-6, 1-4, or 1-2 carbon atoms (e.g., C1-C20, C1-C18, C1-C16, C1-C14, C1-C12, C1-C10, C1-C8, C1-C6, C1-C4, or C1-C2). In some embodiments, the alkenyl, heteroalkenyl, alkynyl, or heteroalkynyl group may contain, e.g., 2-20, 2-18, 2-16, 2-14, 2-12, 2-10, 2-8, 2-6, or 2-4 carbon atoms (e.g., C2-C20, C2-C18, C2-C16. C2-C14, C2-C12, C2-C10, C2-C8, C2-C6, or C2-C4). Examples include, but are not limited to, methyl, ethyl, isobutyl, sec-butyl, tert-butyl, 2-propenyl, and 3-butynyl.

The term “cycloalkyl,” as used herein, represents a monovalent saturated or unsaturated non-aromatic cyclic alkyl group. A cycloalkyl may have, e.g., three to twenty carbons (e.g., a C3-C7, C3-C8, C3-C9, C3-C10, C3-C11, C3-C12, C3-C14, C3-C16, C3-C18, or C3-C20 cycloalkyl). Examples of cycloalkyls include, but are not limited to, cyclopropyl, cyclobutyl, cyclopentyl, cyclohexyl, and cycloheptyl. When the cycloalkyl group includes at least one carbon-carbon double bond, the cycloalkyl group can be referred to as a “cycloalkenyl” group. A cycloalkenyl may have, e.g., four to twenty carbons (e.g., a C4-C7, C4-C8, C4-C9, C4-C10, C4-C11, C4-C12, C4-C14, C4-C16, C4-C18, or C4-C20 cycloalkenyl). Exemplary cycloalkenyl groups include, but are not limited to, cyclopentenyl, cyclohexenyl, and cycloheptenyl. When the cycloalkyl group includes at least one carbon-carbon triple bond, the cycloalkyl group can be referred to as a “cycloalkynyl” group. A cycloalkynyl may have, e.g., eight to twenty carbons (e.g., a C8-C9, C8-C10, C8-C11, C8-C12, C8-C14, C8-C16, C8-C18, or C8-C20 cycloalkynyl). The term “cycloalkyl” also includes a cyclic compound having a bridged multicyclic structure in which one or more carbons bridges two non-adjacent members of a monocyclic ring, e.g., bicyclo[2.2.1.]heptyl and adamantane. The term “cycloalkyl” also includes bicyclic, tricyclic, and tetracyclic fused ring structures, e.g., decalin and spiro cyclic compounds.

The term “aryl,” as used herein, refers to any monocyclic or fused ring bicyclic or tricyclic system which has the characteristics of aromaticity in terms of electron distribution throughout the ring system, e.g., phenyl, naphthyl, or phenanthrene. In some embodiments, a ring system contains 5-15 ring member atoms or 5-10 ring member atoms. An aryl group may have, e.g., five to fifteen carbons (e.g., a C5-C6, C5-C7, C5-C8, C5-C9, C5-C10, C5-C11, C5-C12, C5-C13, C5-C14, or C5-C15 aryl). The term “heteroaryl” also refers to such monocyclic or fused bicyclic ring systems containing one or more, e.g., 1-4, 1-3, 1, 2, 3, or 4, heteroatoms selected from O, S and N. A heteroaryl group may have, e.g., two to fifteen carbons (e.g., a C2-C3, C2-C4, C2-C5, C2-C6, C2-C7, C2-C8, C2-C9. C2-C10, C2-C11, C2-C12, C2-C13, C2-C14, or C2-C15 heteroaryl). The inclusion of a heteroatom permits inclusion of 5-membered rings to be considered aromatic as well as 6-membered rings. Thus, typical heteroaryl systems include, e.g., pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, isoxazolyl, benzoxazolyl, benzoisoxazolyl, and imidazolyl. Because tautomers are possible, a group such as phthalimido is also considered heteroaryl. In some embodiments, the aryl or heteroaryl group is a 5- or 6-membered aromatic rings system optionally containing 1-2 nitrogen atoms. In some embodiments, the aryl or heteroaryl group is an optionally substituted phenyl, pyridyl, indolyl, pyrimidyl, pyridazinyl, benzothiazolyl, benzimidazolyl, pyrazolyl, imidazolyl, isoxazolyl, thiazolyl, or imidazopyridinyl. In some embodiments, the aryl group is phenyl. In some embodiments, an aryl group may be optionally substituted with a substituent such an aryl substituent, e.g., biphenyl.

The term “alkaryl,” refers to an aryl group that is connected to an alkylene, alkenylene, or alkynylene group. In general, if a compound is attached to an alkaryl group, the alkylene, alkenylene, or alkynylene portion of the alkaryl is attached to the compound. In some embodiments, an alkaryl is C6-C35 alkaryl (e.g., C6-C16, C6-C14, C6-C12. C6-C10, C6-C9, C8-C8, C7, or C6 alkaryl), in which the number of carbons indicates the total number of carbons in both the aryl portion and the alkylene, alkenylene, or alkynylene portion of the alkaryl. Examples of alkaryls include, but are not limited to, (C1-C8)alkylene(C8-C12)aryl, (C2-C8)alkenylene(C6-C12)aryl, or (C2-C8)alkynylene(C6-C12)aryl. In some embodiments, an alkaryl is benzyl or phenethyl. In a heteroalkaryl, one or more heteroatoms selected from N, O, and S may be present in the alkylene, alkenylene, or alkynylene portion of the alkaryl group and/or may be present in the aryl portion of the alkaryl group. In an optionally substituted alkaryl, the substituent may be present on the alkylene, alkenylene, or alkynylene portion of the alkaryl group and/or may be present on the aryl portion of the alkaryl group.

The term “amino,” as used herein, represents —N(R^{x})₂ or —N⁺(R^{x})₃, where each R^{x} is, independently, H, alkyl, alkenyl, alkynyl, aryl, alkaryl, cycloalkyl, or two R^{x} combine to form a heterocycloalkyl. In some embodiment, the amino group is —NH₂.

The term “alkamino,” as used herein, refers to an amino group, described herein, that is attached to an alkylene (e.g., C1-C5 alkylene), alkenylene (e.g., C2-C5 alkenylene), or alkynylene group (e.g., C2-C5 alkenylene). In general, if a compound is attached to an alkamino group, the alkylene, alkenylene, or alkynylene portion of the alkamino is attached to the compound. The amino portion of an alkamino refers to —N(R^{x})₂ or —N⁺(R^{x})₃, where each R^{x} is, independently, H, alkyl, alkenyl, alkynyl, aryl, alkaryl, cycloalkyl, or two R^{x} combine to form a heterocycloalkyl. In some embodiment, the amino portion of an alkamino is —NH₂. An example of an alkamino group is C1-C5 alkamino, e.g., C2 alkamino (e.g., CH₂CH₂NH₂ or CH₂CH₂N(CH₃)₂). In a heteroalkamino group, one or more, e.g., 1-4, 1-3, 1, 2, 3, or 4, heteroatoms selected from N, O, and S may be present in the alkylene, alkenylene, or alkynylene portion of the heteroalkamino group. In some embodiments, an alkamino group may be optionally substituted. In a substituted alkamino group, the substituent may be present on the alkylene, alkenylene, or alkynylene portion of the alkamino group and/or may be present on the amino portion of the alkamino group.

The term “alkamide,” as used herein, refers to an amide group that is attached to an alkylene (e.g., C1-C5 alkylene), alkenylene (e.g., C2-C5 alkenylene), or alkynylene (e.g., C2-C5 alkenylene) group. In general, if a compound is attached to an alkamide group, the alkylene, alkenylene, or alkynylene portion of the alkamide is attached to the compound. The amide portion of an alkamide refers to —C(O)—N(R^{x})₂, where each R^{x} is, independently, H, alkyl, alkenyl, alkynyl, aryl, alkaryl, cycloalkyl, or two R^{x} combine to form a heterocycloalkyl. In some embodiment, the amide portion of an alkamide is —C(O)NH₂. An alkamide group may be —(CH₂)₂—C(O)NH₂ or —CH₂—C(O)NH₂. In a heteroalkamide group, one or more, e.g., 1-4, 1-3, 1, 2, 3, or 4, heteroatoms selected from N, O, and S may be present in the alkylene, alkenylene, or alkynylene portion of the heteroalkamide group. In some embodiments, an alkamide group may be optionally substituted. In a substituted alkamide group, the substituent may be present on the alkylene, alkenylene, or alkynylene portion of the alkamide group and/or may be present on the amide portion of the alkamide group.

The terms “alkylene,” “alkenylene,” and “alkynylene,” as used herein, refer to divalent groups having a specified size. In some embodiments, an alkylene may contain, e.g., 1-20, 1-18, 1-16, 1-14, 1-12, 1-10, 1-8, 1-6, 1-4, or 1-2 carbon atoms (e.g., C1-C20, C1-C18, C1-C16, C1-C14, C1-C12, C1-C10, C1-C8, C1-C6, C1-C4, or C1-C2). In some embodiments, an alkenylene or alkynylene may contain, e.g., 2-20, 2-18, 2-16, 2-14, 2-12, 2-10, 2-8, 2-6, or 2-4 carbon atoms (e.g., C2-C20, C2-C18, C2-C16, C2-C14, C2-C12. C2-C10, C2-C8, C2-C6, or C2-C4). Alkylene, alkenylene, and/or alkynylene includes straight-chain and branched-chain forms, as well as combinations of these. The divalency of an alkylene, alkenylene, or alkynylene group does not include the optional substituents on the alkylene, alkenylene, or alkynylene group. For example, two cyclic heptapeptides may be attached to each other by way of a linker that includes alkylene, alkenylene, and/or alkynylene, or combinations thereof. Each of the alkylene, alkenylene, and/or alkynylene groups in the linker is considered divalent with respect to the two attachments on either end of alkylene, alkenylene, and/or alkynylene group. For example, if a linker includes—(optionally substituted alkylene)-(optionally substituted alkenylene)-(optionally substituted alkylene)-, the alkenylene is considered divalent with respect to its attachments to the two alkylenes at the ends of the linker. The optional substituents on the alkenylene are not included in the divalency of the alkenylene. The divalent nature of an alkylene, alkenylene, or alkynylene group (e.g., an alkylene, alkenylene, or alkynylene group in a linker) refers to both of the ends of the group and does not include optional substituents that may be present in an alkylene, alkenylene, or alkynylene group. Because they are divalent, they can link together multiple (e.g., two) parts of a conjugate, e.g., a first cyclic heptapeptide and a second cyclic heptapeptide. Alkylene, alkenylene, and/or alkynylene groups can be substituted by the groups typically suitable as substituents for alkyl, alkenyl and alkynyl groups as set forth herein. For example, C═O is a C1 alkylene that is substituted by an oxo (═O). For example, —HCR—C≡C— may be considered as an optionally substituted alkynylene and is considered a divalent group even though it has an optional substituent, R. Heteroalkylene, heteroalkenylene, and/or heteroalkynylene groups refer to alkylene, alkenylene, and/or alkynylene groups including one or more, e.g., 1-4, 1-3, 1, 2, 3, or 4, heteroatoms, e.g., N, O, and S. For example, a polyethylene glycol (PEG) polymer or a PEG unit —(CH₂)₂—O— in a PEG polymer is considered a heteroalkylene containing one or more oxygen atoms.

The term “cycloalkylene,” as used herein, refers to a divalent cyclic group linking together two parts of a compound. For example, one carbon within the cycloalkylene group may be linked to one part of the compound, while another carbon within the cycloalkylene group may be linked to another part of the compound. A cycloalkylene group may include saturated or unsaturated non-aromatic cyclic groups. A cycloalkylene may have, e.g., three to twenty carbons in the cyclic portion of the cycloalkylene (e.g., a C3-C7, C3-C8, C3-C9, C3-C10, C3-C11, C3-C12, C3-C14, C3-C16, C3-C18, or C3-C20 cycloalkylene). When the cycloalkylene group includes at least one carbon-carbon double bond, the cycloalkylene group can be referred to as a “cycloalkenylene” group. A cycloalkenylene may have, e.g., four to twenty carbons in the cyclic portion of the cycloalkenylene (e.g., a C4-C7, C4-C8, C4-C9. C4-C10, C4-C11, C4-C12, C4-C14, C4-C16, C4-C18, or C4-C20 cycloalkenylene). When the cycloalkylene group includes at least one carbon-carbon triple bond, the cycloalkylene group can be referred to as a “cycloalkynylene” group. A cycloalkynylene may have, e.g., four to twenty carbons in the cyclic portion of the cycloalkynylene (e.g., a C4-C7, C4-C8, C4-C9. C4-C10, C4-C11, C4-C12, C4-C14, C4-C16, C4-C18, or C8-C20 cycloalkynylene). A cycloalkylene group can be substituted by the groups typically suitable as substituents for alkyl, alkenyl and alkynyl groups as set forth herein. Heterocycloalkylene refers to a cycloalkylene group including one or more, e.g., 1-4, 1-3, 1, 2, 3, or 4, heteroatoms, e.g., N, O, and S. Examples of cycloalkylenes include, but are not limited to, cyclopropylene and cyclobutylene. A tetrahydrofuran may be considered as a heterocycloalkylene.

The term “arylene,” as used herein, refers to a multivalent (e.g., divalent or trivalent) aryl group linking together multiple (e.g., two or three) parts of a compound. For example, one carbon within the arylene group may be linked to one part of the compound, while another carbon within the arylene group may be linked to another part of the compound. An arylene may have, e.g., five to fifteen carbons in the aryl portion of the arylene (e.g., a C5-C6, C5-C7, C5-C8, C5-C9, C5-C10, C5-C11, C5-C12, C5-C13, C5-C14, or C5-C15 arylene). An arylene group can be substituted by the groups typically suitable as substituents for alkyl, alkenyl and alkynyl groups as set forth herein. Heteroarylene refers to an aromatic group including one or more, e.g., 1-4, 1-3, 1, 2, 3, or 4, heteroatoms, e.g., N, O, and S. A heteroarylene group may have, e.g., two to fifteen carbons (e.g., a C2-C3, C2-C4, C2-C5, C2-C6, C2-C7, C2-C8, C2-C9, C2-C10, C2-C11, C2-C12, C2-C13, C2-C14, or C2-C15 heteroarylene).

The term “optionally substituted,” as used herein, refers to having 0, 1, or more substituents, such as 0-25, 0-20, 0-10 or 0-5 substituents. Substituents include, but are not limited to, alkyl, alkenyl, alkynyl, aryl, alkaryl, acyl, heteroaryl, heteroalkyl, heteroalkenyl, heteroalkynyl, heteroalkaryl, halogen, oxo, cyano, nitro, amino, alkamino, hydroxy, alkoxy, alkanoyl, carbonyl, carbamoyl, guanidinyl, ureido, amidinyl, any of the groups or moieties described above, and hetero versions of any of the groups or moieties described above. Substituents include, but are not limited to, F, Cl, methyl, phenyl, benzyl, OR, NR₂, SR, SOR, SO₂R, OCOR, NRCOR, NRCONR₂, NRCOOR, OCONR₂, RCO, COOR, alkyl-OOCR, SO₃R, CONR₂, SO₂NR₂, NRSO₂NR₂, CN, CF₃, OCF₃, SiR₃, and NO₂, wherein each R is, independently, H, alkyl, alkenyl, aryl, heteroalkyl, heteroalkenyl, or heteroaryl, and wherein two of the optional substituents on the same or adjacent atoms can be joined to form a fused, optionally substituted aromatic or nonaromatic, saturated or unsaturated ring which contains 3-8 members, or two of the optional substituents on the same atom can be joined to form an optionally substituted aromatic or nonaromatic, saturated or unsaturated ring which contains 3-8 members.

An optionally substituted group or moiety refers to a group or moiety (e.g., any one of the groups or moieties described above) in which one of the atoms (e.g., a hydrogen atom) is optionally replaced with another substituent. For example, an optionally substituted alkyl may be an optionally substituted methyl, in which a hydrogen atom of the methyl group is replaced by, e.g., OH. As another example, a substituent on a heteroalkyl or its divalent counterpart, heteroalkylene, may replace a hydrogen on a carbon or a hydrogen on a heteroatom such as N. For example, the hydrogen atom in the group —R—NH—R— may be substituted with an alkamide substituent, e.g., —R—N[(CH₂C(O)N(CH₃)₂]—R. Generally, an optional substituent is a noninterfering substituent. A “noninterfering substituent” refers to a substituent that leaves the ability of the conjugates described herein (e.g., conjugates of any one of formulas (I)-(XXXVI)) to either bind to lipopolysaccharides (LPS) or to kill or inhibit the growth of Gram-negative bacteria qualitatively intact. Thus, in some embodiments, the substituent may alter the degree of such activity. However, as long as the conjugate retains the ability to bind to LPS and/or to kill or inhibit the growth of Gram-negative bacteria, the substituent will be classified as “noninterfering.” In some aspects, a noninterfering substituent leaves the ability of a conjugate described herein (e.g., a conjugate of any one of formulas (I)-(XXXVI)) to kill or inhibit the growth of Gram-negative bacteria qualitatively intact as determined by measuring the minimum inhibitory concentration (MIC) against at least one Gram-negative bacteria as known in the art, wherein the MIC is 128 μg/mL or less. In some aspects, a noninterfering substituent leaves the ability of a conjugate described herein (e.g., a conjugate of any one of formulas (I)-(XXXVI)) to bind to lipopolysaccharides (LPS) from the cell membrane of Gram-negative bacteria qualitatively intact, as determined by an LPS binding assay, wherein the conjugate shows a value of about 10% or greater displacement of a fluorogenic substrate at 250 μM of the conjugate.

The term “hetero,” when used to describe a chemical group or moiety, refers to having at least one heteroatom that is not a carbon or a hydrogen, e.g., N, O, and S. Any one of the groups or moieties described above may be referred to as hetero if it contains at least one heteroatom. For example, a heterocycloalkyl, heterocycloalkenyl, or heterocycloalkynyl group refers to a cycloalkyl, cycloalkenyl, or cycloalkynyl group that has one or more heteroatoms independently selected from, e.g., N, O, and S. An example of a heterocycloalkenyl group is a maleimido. For example, a heteroaryl group refers to an aromatic group that has one or more heteroatoms independently selected from, e.g., N, O, and S. One or more heteroatoms may also be included in a substituent that replaced a hydrogen atom in a group or moiety as described herein. For example, in an optionally substituted heteroaryl group, if one of the hydrogen atoms in the heteroaryl group is replaced with a substituent (e.g., methyl), the substituent may also contain one or more heteroatoms (e.g., methanol).

The term “acyl,” as used herein, refers to a group having the structure:

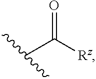

wherein R^{z} is an optionally substituted alkyl, alkenyl, alkynyl, cycloalkyl, cycloalkenyl, cycloalkynyl, aryl, alkaryl, alkamino, heteroalkyl, heteroalkenyl, heteroalkynyl, heterocycloalkyl, heterocycloalkenyl, heterocycloalkynyl, heteroaryl, heteroalkaryl, or heteroalkamino.

The term “halo” or “halogen.” as used herein, refers to any halogen atom, e.g., F, Cl, Br, or I. Any one of the groups or moieties described herein may be referred to as a “halo moiety” if it contains at least one halogen atom, such as haloalkyl.

The term “hydroxyl,” as used herein, represents an —OH group.

The term “oxo,” as used herein, refers to a substituent having the structure ═O, where there is a double bond between an atom and an oxygen atom.

The term “carbonyl,” as used herein, refers to a group having the structure:

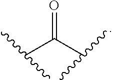 [SMILES: *C(*)=O]

The term “thiocarbonyl,” as used herein, refers to a group having the structure:

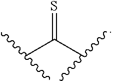 [SMILES: *C(*)=S]

The term “phosphate,” as used herein, represents the group having the structure:

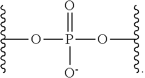 [SMILES: *OP(=O)([O-])O*]

The term “phosphoryl,” as used herein, represents the group having the structure:

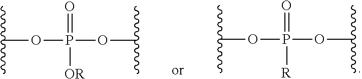

The term “sulfonyl,” as used herein, represents the group having the structure:

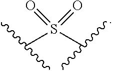 [SMILES: *S(*)(=O)=O]

The term “imino,” as used herein, represents the group having the structure:

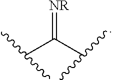

wherein R is an optional substituent.

The term “N-protecting group.” as used herein, represents those groups intended to protect an amino group against undesirable reactions during synthetic procedures. Commonly used N-protecting groups are disclosed in Greene, “Protective Groups in Organic Synthesis,” 5th Edition (John Wiley & Sons. New York, 2014), which is incorporated herein by reference. N-protecting groups include, e.g., acyl, aryloyl, and carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, carboxybenzyl (CBz), 4-chlorobenzoyl, 4-bromobenzoyl, 4-nitrobenzoyl, and chiral auxiliaries such as protected or unprotected D, L or D, L-amino acid residues such as alanine, leucine, phenylalanine; sulfonyl-containing groups such as benzenesulfonyl and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyl oxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl (BOC), diisopropylmethoxycarbonyl, isopropyloxycarbonyl, ethoxycarbonyl, methoxycarbonyl, allyloxycarbonyl, 2,2,2-trichloroethoxycarbonyl, phenoxycarbonyl, 4-nitrophenoxy carbonyl, fluorenyl-9-methoxycarbonyl (Fmoc), cyclopentyloxycarbonyl, adamantyloxycarbonyl, cyclohexyloxycarbonyl, and phenylthiocarbonyl; alkaryl groups such as benzyl, triphenylmethyl, and benzyloxymethyl; and silyl groups such as trimethylsilyl.

The term “amino acid,” as used herein, means naturally occurring amino acids and non-naturally occurring amino acids.

The term “naturally occurring amino acids,” as used herein, means amino acids including Ala, Arg, Asn, Asp, Cys, Gln, Glu, Gly, His, Ile, Leu, Lys, Met, Phe, Pro, Ser, Thr, Trp, Tyr, and Val.

The term “non-naturally occurring amino acid,” as used herein, means an alpha amino acid that is not naturally produced or found in a mammal. Examples of non-naturally occurring amino acids include D-amino acids; an amino acid having an acetylaminomethyl group attached to a sulfur atom of a cysteine; a pegylated amino acid; the omega amino acids of the formula NH₂(CH₂)ₙCOOH where n is 2-6, neutral nonpolar amino acids, such as sarcosine, t-butyl alanine, t-butyl glycine, N-methyl isoleucine, and norleucine; oxymethionine; phenylglycine; citrulline; methionine sulfoxide; cysteic acid; omithine; diaminobutyric acid; 3-aminoalanine; 3-hydroxy-D-proline; 2,4-diaminobutyric acid; 2-aminopentanoic acid; 2-aminooctanoic acid, 2-carboxy piperazine; piperazine-2-carboxylic acid, 2-amino-4-phenylbutanoic acid; 3-(2-naphthyl)alanine, and hydroxyproline. Other amino acids are α-aminobutyric acid, α-amino-α-methylbutyrate, aminocyclopropane-carboxylate, aminoisobutyric acid, aminonorbornyl-carboxylate, L-cyclohexylalanine, cyclopentylalanine, L-N-methylleucine, L-N-methylmethionine, L-N-methylnorvaline, L-N-methylphenylalanine, L-N-methylproline, L-N-methylserine, L-N-methyltryptophan, D-omithine, L-N-methylethylglycine, L-norleucine, α-methyl-aminoisobutyrate, α-methylcyclohexylalanine, D-α-methylalanine, D-α-methylarginine, D-α-methylasparagine, D-α-methylaspartate, D-α-methylcysteine, D-α-methylglutamine, D-α-methylhistidine, D-α-methylisoleucine, D-α-methylleucine, D-α-methyllysine, D-α-methylmethionine, D-α-methylornithine, D-α-methylphenylalanine, D-α-methylproline, D-α-methylserine, D-N-methylserine, D-α-methylthreonine, D-α-methyltryptophan, D-α-methyltyrosine, D-α-methylvaline, D-N-methylalanine, D-N-methylarginine, D-N-methylasparagine, D-N-methylaspartate, D-N-methylcysteine, D-N-methylglutamine, D-N-methylglutamate, D-N-methylhistidine, D-N-methylisoleucine, D-N-methylleucine, D-N-methyllysine, N-methylcyclohexylalanine, D-N-methylornithine, N-methylglycine, N-methylaminoisobutyrate, N-(1-methylpropyl)glycine, N-(2-methylpropyl)glycine, D-N-methyltryptophan, D-N-methyltyrosine, D-N-methylvaline, γ-aminobutyric acid, L-t-butylglycine, L-ethylglycine, L-homophenylalanine, L-α-methylarginine, L-α-methylaspartate, L-α-methylcysteine, L-α-methylglutamine, L-α-methylhistidine, L-α-methylisoleucine, L-α-methylleucine, L-α-methylmethionine, L-α-methylnorvaline, L-α-methylphenylalanine, L-α-methylserine, L-α-methyltryptophan, L-α-methylvaline, N-(N-(2,2-diphenylethyl) carbamylmethylglycine, 1-carboxy-1-(2,2-diphenyl-ethylamino) cyclopropane, 4-hydroxyproline, omithine, 2-aminobenzoyl (anthraniloyl), D-cyclohexylalanine, 4-phenyl-phenylalanine, L-citrulline, α-cyclohexylglycine, L-1,2,3,4-tetrahydroisoquinoline-3-carboxylic acid, L-thiazolidine-4-carboxylic acid, L-homotyrosine, L-2-furylalanine, L-histidine (3-methyl), N-(3-guanidinopropyl)glycine, O-methyl-L-tyrosine, O-glycan-serine, meta-tyrosine, nor-tyrosine, L-N,N′,N″-trimethyllysine, homolysine, norlysine, N-glycan asparagine, 7-hydroxy-1,2,3,4-tetrahydro-4-fluorophenylalanine, 4-methylphenylalanine, bis-(2-picolyl)amine, pentafluorophenylalanine, indoline-2-carboxylic acid, 2-aminobenzoic acid, 3-amino-2-naphthoic acid, asymmetric dimethylarginine, L-tetrahydroisoquinoline-1-carboxylic acid, D-tetrahydroisoquinoline-1-carboxylic acid, 1-amino-cyclohexane acetic acid, D/L-allylglycine, 4-aminobenzoic acid, 1-amino-cyclobutane carboxylic acid, 2 or 3 or 4-aminocyclohexane carboxylic acid, 1-amino-1-cyclopentane carboxylic acid, 1-aminoindane-1-carboxylic acid, 4-amino-pyrrolidine-2-carboxylic acid, 2-aminotetraline-2-carboxylic acid, azetidine-3-carboxylic acid, 4-benzyl-pyrolidine-2-carboxylic acid, tert-butylglycine, b-(benzothiazolyl-2-yl)-alanine, b-cyclopropyl alanine, 5,5-dimethyl-1,3-thiazolidine-4-carboxylic acid, (2R,4S)4-hydroxypiperidine-2-carboxylic acid. (2S,4S) and (2S,4R)-4-(2-naphthylmethoxy)-pyrolidine-2-carboxylic acid, (2S,4S) and (2S,4R)4-phenoxy-pyrrolidine-2-carboxylic acid, (2R,5S) and (2S,5R)-5-phenyl-pyrrolidine-2-carboxylic acid, (2S,4S)-4-amino-1-benzoyl-pyrrolidine-2-carboxylic acid, t-butylalanine, (2S,5R)-5-phenyl-pyrrolidine-2-carboxylic acid, 1-aminomethyl-cyclohexane-acetic acid, 3,5-bis-(2-amino)ethoxy-benzoic acid, 3,5-diamino-benzoic acid, 2-methylamino-benzoic acid, N-methylanthranylic acid, L-N-methylalanine, L-N-methylarginine, L-N-methylasparagine, L-N-methylaspartic acid, L-N-methylcysteine, L-N-methylglutamine, L-N-methylglutamic acid, L-N-methylhistidine, L-N-methylisoleucine, L-N-methyllysine. L-N-methylnorieucine, L-N-methylornithine, L-N-methylthreonine, L-N-methyltyrosine, L-N-methylvaline, L-N-methyl-t-butylglycine, L-norvaline, α-methyl-γ-aminobutyrate, 4,4′-biphenylalanine, α-methylcylcopentylalanine, α-methyl-α-napthylalanine, α-methylpenicillamine. N-(4-aminobutyl)glycine, N-(2-aminoethyl)glycine, N-(3-aminopropyl)glycine, N-amino-α-methylbutyrate, α-napthylalanine, N-benzylglycine, N-(2-carbamylethyl)glycine, N-(carbamylmethyl)glycine, N-(2-carboxyethyl)glycine, N-(carboxymethyl)glycine, N-cyclobutylglycine, N-cyclodecylglycine, N-cycloheptylglycine, N-cyclohexylglycine, N-cyclodecylglycine, N-cylcododecylglycine, N-cyclooctylglycine, N-cyclopropylglycine, N-cycloundecylglycine, N-(2,2-diphenylethyl)glycine, N-(3,3-diphenylpropyl)glycine, N-(3-guanidinopropyl)glycine, N-(1-hydroxyethyl)glycine, N-(hydroxyethyl))glycine, N-(imidazolylethyl))glycine, N-(3-indolylyethyl)glycine, N-methyl-γ-aminobutyrate, D-N-methylmethionine, N-methylcyclopentylalanine, D-N-methylphenylalanine, D-N-methylproline, D-N-methylthreonine, N-(1-methylethyl)glycine, N-methyl-napthylalanine, N-methylpenicillamine, N-(p-hydroxyphenyl)glycine, N-(thiomethyl)glycine, penicillamine, L-α-methylalanine, L-α-methylasparagine, L-α-methyl-t-butylglycine, L-methylethylglycine, L-α-methylglutamate, L-α-methylhomophenylalanine, N-(2-methylthioethyl)glycine, L-α-methyllysine, L-α-methylnorieucine, L-α-methylornithine, L-α-methylproline, L-α-methylthreonine, L-α-methyltyrosine, L-N-methyl-homophenylalanine, N-(N-(3,3-diphenylpropyl) carbamylmethylglycine, L-pyroglutamic acid, D-pyroglutamic acid, O-methyl-L-serine, O-methyl-L-homoserine, 5-hydroxylysine, α-carboxyglutamate, phenylglycine, L-pipecolic acid (homoproline), L-homoleucine, L-lysine (dimethyl), L-2-naphthylalanine, L-dimethyldopa or L-dimethoxy-phenylalanine, L-3-pyridylalanine, L-histidine (benzoyloxymethyl), N-cycloheptylglycine, L-diphenylalanine, O-methyl-L-homotyrosine, L-β-homolysine, O-glycan-threoine, Ortho-tyrosine, L-N,N′-dimethyllysine, L-homoarginine, neotryptophan, 3-benzothienylalanine, isoquinoline-3-carboxylic acid, diaminopropionic acid, homocysteine, 3,4-dimethoxyphenylalanine, 4-chlorophenylalanine, L-1,2,3,4-tetrahydronorharman-3-carboxylic acid, adamantylalanine, symmetrical dimethylarginine, 3-carboxythiomorpholine, D-1,2,3,4-tetrahydronorharman-3-carboxylic acid, 3-aminobenzoic acid, 3-amino-1-carboxymethyl-pyridin-2-one, 1-amino-1-cyclohexane carboxylic acid, 2-aminocyclopentane carboxylic acid, 1-amino-1-cyclopropane carboxylic acid, 2-aminoindane-2-carboxylic acid, 4-amino-tetrahydrothiopyran-4-carboxylic acid, azetidine-2-carboxylic acid, b-(benzothiazol-2-yl)-alanine, neopentylglycine, 2-carboxymethyl piperidine, b-cyclobutyl alanine, allylglycine, diaminopropionic acid, homo-cyclohexyl alanine, (2S,4R)-4-hydroxypiperidine-2-carboxylic acid, octahydroindole-2-carboxylic acid, (2S,4R) and (2S,4R)-4-(2-naphthyl), pyrrolidine-2-carboxylic acid, nipecotic acid, (2S,4R) and (2S,4S)-4-(4-phenylbenzyl) pyrrolidine-2-carboxylic acid, (3S)-1-pyrrolidine-3-carboxylic acid, (2S,4S)-4-tritylmercapto-pyrrolidine-2-carboxylic acid, (2S,4S)-4-mercaptoproline, t-butylglycine, N,N-bis(3-aminopropyl)glycine, 1-amino-cyclohexane-1-carboxylic acid, N-mercaptoethylglycine, and selenocysteine. In some embodiments, amino acid residues may be charged or polar. Charged amino acids include alanine, lysine, aspartic acid, or glutamic acid, or non-naturally occurring analogs thereof. Polar amino acids include glutamine, asparagine, histidine, serine, threonine, tyrosine, methionine, or tryptophan, or non-naturally occurring analogs thereof.

It is specifically contemplated that in some embodiments, a terminal amino group in the amino acid may be an amido group or a carbamate group.

The term “antibacterial agent,” as used herein, refers to an agent that is used in addition to one or more of the conjugates described herein (e.g., conjugates of any one of formulas (I)-(XXXVI)) in methods of treating a bacterial infection (e.g., Gram-negative bacterial infection) and/or preventing, stabilizing, or inhibiting the growth of bacteria, or killing bacteria. An antibacterial agent may be an agent that prevents the entrance of a bacteria (e.g., a Gram-negative bacteria) into a subject's cells, tissues, or organs, inhibits the growth of a bacteria (e.g., a Gram-negative bacteria) in a subject's cells, tissues, or organs, and/or kills a bacteria (e.g., a Gram-negative bacteria) that is inside a subject's cells, tissues, or organs. Examples of antibacterial agents are described in detail further herein. In some embodiments, an antibacterial agent used in addition to a conjugate described herein is linezolid or tedizolid (e.g., tedizolid phosphate).

The term “bacterial infection,” as used herein, refers to the invasion of a subject's cells, tissues, and/or organs by bacteria (e.g., Gram-negative bacteria), thus, causing an infection. In some embodiments, the bacteria may grow, multiply, and/or produce toxins in the subject's cells, tissues, and/or organs. In some embodiments, a bacterial infection can be any situation in which the presence of a bacterial population(s) is latent within or damaging to a host body. Thus, a subject is “suffering” from a bacterial infection when a latent bacterial population is detectable in or on the subject's body, an excessive amount of a bacterial population is present in or on the subject's body, or when the presence of a bacterial population(s) is damaging the cells, tissues, and/or organs of the subject.

The term “protecting against,” as used herein, refers to preventing a subject from developing a bacterial infection (e.g., a Gram-negative bacterial infection) or decreasing the risk that a subject may develop a bacterial infection (e.g., a Gram-negative bacterial infection). Prophylactic drugs used in methods of protecting against a bacterial infection in a subject are often administered to the subject prior to any detection of the bacterial infection. In some embodiments of methods of protecting against a bacterial infection, a subject (e.g., a subject at risk of developing a bacterial infection) may be administered a conjugate described herein (e.g., a conjugate having any one of formulas (I)-(XXXVI)) to prevent the bacterial infection development or decrease the risk of the bacterial infection development.

The term “treating” or “to treat,” as used herein, refers to a therapeutic treatment of a bacterial infection (e.g., a Gram-negative bacterial infection) in a subject. In some embodiments, a therapeutic treatment may slow the progression of the bacterial infection, improve the subject's outcome, and/or eliminate the infection. In some embodiments, a therapeutic treatment of a bacterial infection (e.g., a Gram-negative bacterial infection) in a subject may alleviate or ameliorate of one or more symptoms or conditions associated with the bacterial infection, diminish the extent of the bacterial infection, stabilize (i.e., not worsening) the state of the bacterial infection, prevent the spread of the bacterial infection, and/or delay or slow the progress of the bacterial infection, as compare the state and/or the condition of the bacterial infection in the absence of the therapeutic treatment.

The phrase “LPS-induced nitric oxide (NO) production from a macrophage,” as used herein, refers to the ability of the lipopolysaccharides (LPS) in Gram-negative bacteria to activate a macrophage and induce NO production from the macrophage. NO production from a macrophage in response to LPS is a signal of macrophage activation, which may lead to sepsis in a subject, e.g., a Gram-negative bacteria infected subject. The disclosure features conjugates (e.g., conjugates of any one of formulas (I)-(XXXVI)) that are able to bind to LPS in the cell membrane of Gram-negative bacteria to disrupt and permeabilize the cell membrane, thus neutralizing an immune response to LPS. NO production from a macrophage may be measured using available techniques in the art, e.g., a Griess assay.

The term “resistant strain of bacteria,” as used herein, refers to a strain of bacteria (e.g., Gram-negative or Gram-positive bacteria) that is refractory to treatment with an antibiotic, such as an antibiotic described in the Detailed Description. Antibiotics that a strain of bacteria is resistant to do not include the conjugates described herein (e.g., conjugates of any one of formulas (I)-(XXXVI)). Resistance may arise through natural resistance in certain types of bacteria, spontaneous random genetic mutations, and/or by inter- or intra-species horizontal transfer of resistance genes. In some embodiments, a resistant strain of bacteria contains an mcr-1 gene, an mcr-2 gene, an mcr-3 gene, an mcr-4 gene, an mcr-5 gene, an mcr-6 gene, an ma-7 gene, and/or an mcr-8 gene. In some embodiments, a resistant strain of bacteria contains a chromosomal mutation conferring polymyxin resistance. In some embodiments, a resistant strain of bacteria contains an mcr-1 gene, an mcr-2 gene, an mcr-3 gene, an mcr-4 gene, an mcr-5 gene, an mcr-8 gene, an mcr-7 gene, and/or an mcr-8 gene in combination with other antibiotic resistance genes. In some embodiments, a resistant strain of bacteria is a resistant strain of E. coli (e.g., E. coli BAA-2469).

The term “activating an immune cell,” as used herein, refers to the ability of a conjugate to bind to an immune cell to produce an effective immune response. The ability of a conjugate to directly or indirectly bind to an immune cell to produce an effective immune response may be quantified by measuring the concentration of the conjugate at which such immune response is produced. In some embodiments, the concentration of a conjugate that binds to an FcγR receptor on an immune cell to trigger an effective immune response may be less than or equal to 10,000 nM as measured in accordance with, e.g., an enzyme-linked immunosorbent assay (ELISA). In some embodiments, the concentration of a conjugate that binds to an immune cell receptor to trigger an effective immune response may be less than or equal to 1000 nM or less than or equal to 100 nM as measured in accordance with an ELISA. In an ELISA, an FcγR may be immobilized on a support or surface using conventional techniques in the art. After the FcγR is immobilized to the surface, a conjugate described herein may be applied over the surface so it is captured by the FcγR through binding of the Fc domain in the conjugate to the FcγR. In some embodiments, the conjugate may be detected using a secondary antibody, which is linked to an enzyme (e.g., horseradish peroxidase) for subsequent signal amplification. During signal amplification, the enzyme's substrate (e.g., 3,3′-diaminobenzidine) may be added to produce a measurable signal (e.g., color change).

The term “average value of T,” as used herein, refers to the mean number of monomers of cyclic heptapeptides or dimers of cyclic heptapeptides conjugated to an Fc domain monomer within a population of conjugates. In some embodiments, within a population of conjugates, the average number of monomers of cyclic heptapeptides or dimers of cyclic heptapeptides conjugated to an Fc domain monomer may be from 1 to 5 (e.g., 1 to 2).

The term “subject,” as used herein, can be a human, non-human primate, or other mammal, such as but not limited to dog, cat, horse, cow, pig, turkey, goat, fish, monkey, chicken, rat, mouse, and sheep.

The term “substantially simultaneously,” as used herein, refers to two or more events that occur at the same time or within a narrow time frame of each other. As disclosed herein, a conjugate described herein (e.g., a conjugate of any one of formulas (I)-(XXXVI)) and an antibacterial agent (e.g., linezolid or tedizolid) may be administered substantially simultaneously, which means that the conjugate and the antibacterial agent are administered together (e.g., in one pharmaceutical composition) or separately but within a narrow time frame of each other, e.g., within 10 minutes, e.g., 10, 9, 8, 7, 6, 5, 4, 3, 2, or 1 minute, or 45, 30, 15, or 10 seconds of each other.

The term “therapeutically effective amount,” as used herein, refers to an amount, e.g., pharmaceutical dose, effective in inducing a desired effect in a subject or in treating a subject having a condition or disorder described herein (e.g., a bacterial infection (e.g., a Gram-negative bacterial infection)). It is also to be understood herein that a “therapeutically effective amount” may be interpreted as an amount giving a desired therapeutic and/or preventative effect, taken in one or more doses or in any dosage or route, and/or taken alone or in combination with other therapeutic agents (e.g., an antibacterial agent described herein). For example, in the context of administering a conjugate described herein (e.g., a conjugate of any one of formulas (I)-(XXXVI)) that is used for the treatment of a bacterial infection, an effective amount of a conjugate is, for example, an amount sufficient to prevent, slow down, or reverse the progression of the bacterial infection as compared to the response obtained without administration of the conjugate.

As used herein, the term “pharmaceutical composition” refers to a medicinal or pharmaceutical formulation that contains at least one active ingredient (e.g., a conjugate of any one of formulas (I)-(XXXVI)) as well as one or more excipients and diluents to enable the active ingredient suitable for the method of administration. The pharmaceutical composition of the present disclosure includes pharmaceutically acceptable components that are compatible with a conjugate described herein (e.g., a conjugate of any one of formulas (I)-(XXXVI)).

As used herein, the term “pharmaceutically acceptable carrier” refers to an excipient or diluent in a pharmaceutical composition. For example, a pharmaceutically acceptable carrier may be a vehicle capable of suspending or dissolving the active conjugate (e.g., a conjugate of any one of formulas (I)-(XXXVI)). The pharmaceutically acceptable carrier must be compatible with the other ingredients of the formulation and not deleterious to the recipient. In the present disclosure, the pharmaceutically acceptable carrier must provide adequate pharmaceutical stability to a conjugate described herein. The nature of the carrier differs with the mode of administration. For example, for oral administration, a solid carrier is preferred; for intravenous administration, an aqueous solution carrier (e.g., WFI, and/or a buffered solution) is generally used.

The term “pharmaceutically acceptable salt,” as used herein, represents salts of the conjugates described herein (e.g., conjugates of any one of formulas (I)-(XXXVI)) that are, within the scope of sound medical judgment, suitable for use in methods described herein without undue toxicity, irritation, and/or allergic response. Pharmaceutically acceptable salts are well known in the art. For example, pharmaceutically acceptable salts are described in: Pharmaceutical Salts: Properties, Selection, and Use (Eds. P. H. Stahl and C. G. Wermuth), Wiley-VCH, 2008. The salts can be prepared in situ during the final isolation and purification of the conjugates described herein or separately by reacting the free base group with a suitable organic acid.

The term “about,” as used herein, indicates a deviation of *5%. For example, about 10% refers to from 9.5% to 10.5%.

Definitions of abbreviations used in the disclosure are provided in Table A below:

TABLE A

| Abbreviation | Stands for |
|---|---|
| CAN | acetonitrile |
| CBZ, Cbz | carboxybenzyl |
| DCHA | dicyclohexylamine |
| DCM | dichloromethane |
| DIEA, DIPEA | N,N-diisopropylethylamine |
| DMF | dimethylformamide |
| EDC, EDCI | 1-ethyl-3-(3-dimethylaminopropyl) carbodiimide |
| EDTA | ethylenediaminetetraacetic acid |
| FMOC | fluorenylmethyloxy carbonyl |
| HATU | 1-[Bis(dimethylamino)methylene]-1H-1 2,3- |
| | triazolo[4,5-b]pyridinium 3-oxid |
| | hexafluorophosphate |
| HOBT | hydroxybenzotriazole |
| PMBN | polymyxin B nonapeptide |
| PMEN | polymyxin E nonapeptide |
| RPLC | reversed phase liquid chromatography |
| TEA | triethylamine |
| TFA | trifluoroacetic acid |
| THF | tetrahydrofuran |

It will be understood that in any of the above-described aspects or embodiments of the invention, that an Fc-domain-containing composition may be substituted for an Fc domain and an Fc-domain-monomer-containing composition may be substituted for an Fc domain monomer in any of the conjugates of the invention.

Accordingly, the invention also features a polymyxin dimer or a polymyxin monomer of the invention conjugated (e.g., via a linker, such as a linker described herein) to an Fc-domain-containing composition. Fc-monomer-domain-containing compositions and Fc-domain-containing compositions of the invention include a polypeptide including and Fc domain monomer; a composition including two or more polypeptides (e.g., two or more polypeptide linked via one or more disulfide bonds), wherein one or more of polypeptides includes an Fc domain monomer; or any protein including one or more Fe domain monomers.

In some embodiments, an Fc-domain-containing composition may be substituted for an Fc domain and an Fc-domain-monomer-containing composition may be substituted for an Fc domain monomer in any one of formulas (I)-(XXXVI) (e.g., any one of formulas D-IIa, D-IIb, D-IIc, D-IId, D-IIe, D-IIIa, D-IIIb, D-IIIc, D-IIId, D-IVa, D-IVa-1, D-IVa-2, D-IVa-3, D-IVb, D-IVb-1, D-IVc, D-IVc-1, D-IVd, D-V, D-Va, D-Vb, D-Vc, D-V-1, D-V-2, D-V-3, D-V-4, D-V-5, D-V-6, D-V-7, D-V-8, D-V-9, D-V-10, D-V-11, D-V-12, D-V-13, D-V-14, D-VI, D-VIa, D-VIb, D-VIc, D-VId, D-VI-1, D-VI-2, D-VI-3, D-VI-4, D-VI-5, D-VI-6, D-VI-7, D-VI-8, D-VI-9, D-VI-10, D-VI-11, D-VI-12, D-VI-13, D-VI-14, D-VI-15, D-VI-16, D-VII, D-VIII, D-IX, D-IX-1, D-IX-2, D-IX-3, D-IX-4, D-X, D-XI, D-XI-1, D-XI-2, D-XI-2, D-XII, D-XII-1, D-XIII, D-XIV, D-XXIX, D-XXIXa, D-XXX, D-XXXI, D-XXXI-1, D-XXXI-2, D-XXXI-3, D-XXXII, D-XXXIII, D-XXXIV, D-XXXIV-1, D-XXXIV-2, D-XXXIV-3, XXXIV-4, D-XXXIV-5, D-XXXIV-8, D-XXXIV-7, D-XXXIV-8, D-XXXIV-9, D-XXXV, D-XXXVI, D-XXXVI-1, D-XXXVI-2, D-XV, D-XVI, D-XVII, D-XVIII, D-XIX, D-XX, D-XXI, D-XXII, D-XXXIII, D-XXIV, D-XXV, D-XXVI, D-XXVII, D-XXVIII, D-XXVIII-1, D-XXVIII-2, M-II, M-III, M-IV, M-IV-1, M-IV-2, M-V, M-V-I, M-VI, M-VI-1, M-VII, M-VII-1, M-VII-2, M-VII-3, M-VIII, M-VIII-1, M-VIII-2, M-VIII-3, M-VIII-4, M-VIII-5, M-IX, M-IX-1, M-IX-2, M-IX-3, M-X, M-XI, M-XI-2, M-XII, M-XIII, M-XIII-1, M-XIV, M-XIV-1, M-XV, M-XV-2, M-XVI, M-XVII, M-XVII-1, M-XVII-2, M-XVII-3, M-XVIII, M-XXVII, M-XXVIIa, M-XXVIIb, M-XXVIIc, M-XXVII-1, M-XIX, M-XX, M-XXI, M-XXII, M-XXIV, M-XXV, or M-XXVI). In any of the formulas described herein (e.g., any one of formulas (I)-(XXXVI), when n is 1, E is an Fc-domain-monomer-containing composition. In any of the formulas described herein (e.g., any one of formulas (I)-(XXXVI), when n is 2, E is an Fc-domain-containing composition.

In certain embodiments, the Fc-domain-containing composition is an antibody or an antibody fragment. An antibody may include any form of immunoglobulin, heavy chain antibody, light chain antibody, LRR-based antibody, or other protein scaffold with antibody-like properties, as well as any other immunological binding moiety known in the art, including antibody fragments (e.g., a Fab, Fab′, Fab′2, F(ab′)2, Fd, Fv, Feb, scFv, or SMIP). The subunit structures and three-dimensional configurations of different classes of antibodies are known in the art. An antibody fragment may include a binding moiety that includes a portion derived from or having significant homology to an antibody, such as the antigen-determining region of an antibody. Exemplary antibody fragments include Fab, Fab′, Fab′2, F(ab′)2, Fd, Fv, Feb, scFv, and SMIP.

In particular embodiments, the antibody or antibody fragment is a human, mouse, goat, sheep, rabbit, chicken, guinea pig, hamster, horse, or rat antibody or antibody fragment. In specific embodiments, the antibody is an IgG, IgA, IgD, IgE, IgM, or intrabody. In certain embodiments, the antibody fragment comprises an scFv, sdAb, dAb, Fab, Fab′, Fab′2, F(ab′)2, Fd, Fv, Feb, or SMIP.

In some embodiments, the Fc-domain-containing composition (e.g., an antibody or antibody fragment) confers binding specificity to a one or more targets (e.g., an antigen).

In some embodiments, the one or more targets (e.g., an antigen) bound by the Fc-domain-containing composition (e.g., an antibody or antibody fragment) is a lipopolysaccharide (e.g., a lipoglycan or an endotoxin), TNFα, an interleukin (e.g., IL-1 or IL-2), a toll-like receptor (e.g., TLP-2 or TLR-4), a antibody that recognizes a bacterial surface antigen (e.g., a bacterial surface antigen may be used to confer specificity to bacteria or to a particular strain of bacteria). Many antibodies that bind specifically to bacterial surface antigens are known to those of skill in the art Exemplary antibodies that bind bacterial surface antigens include AR-101 (binds P. aeruginosa lipopolysaccharide), MEDI3902 (binds P. aeruginosa PsL and PcrV), and Shigamab (binds E. coli shiga toxin).

In some embodiments, the Fc-domain-containing composition is an antibody or antibody fragment described in one of the following publications, in of which is hereby incorporated by reference in its entirety: Lima et al., PLoS One. Therapeutic effects of treatment with anti-TLR2 and anti-TLR4 monoclonal antibodies in polymicrobial sepsis DOI:10.1371/journal.pone.0132338 (2015); Strohl R. Protein and Cell. Current progress in innovative engineered antibodies. DOI 10.1007/s13238-017-0457-8 (2017) (see, in particular, compounds numbers 5, 8, 13, 27, 42, 64, 69 of Table 1); Czaplewski, L. et al. Alternatives to antibiotics—a pipeline portfolio review. Lancet. Infec Disease. 16: 239-51 (2016); and Morrison, C. Antibacterial antibodies gain traction. Nature Reviews Drug Discovery. 14:737-38 (2015).

Other features and advantages of the conjugates described herein will be apparent from the following Detailed Description and the claims.

### DESCRIPTION OF THE DRAWINGS

FIG. 1 shows non-reducing and reducing SDS-PAGE and a schematic illustration of an Fc domain formed from Fc domain monomers having the sequence of SEQ ID NO: 1.

FIG. 2 shows non-reducing and reducing SDS-PAGE and a schematic illustration of an Fc domain formed from Fc domain monomers having the sequence of SEQ ID NO: 3.

FIG. 3 shows non-reducing and reducing SDS-PAGE and a schematic illustration of an Fc domain formed from Fc domain monomers having the sequence of SEQ ID NO: 5.

FIG. 4 shows non-reducing and reducing SDS-PAGE and a schematic illustration of an Fc domain formed from Fc domain monomers having the sequence of SEQ ID NO: 7.

FIG. 5 shows non-reducing and reducing SDS-PAGE and a schematic illustration of an Fc domain formed from Fc domain monomers having the sequence of SEQ ID NO: 9.

FIG. 6 shows non-reducing and reducing SDS-PAGE and a schematic illustration of an Fc domain formed from Fc domain monomers having the sequence of SEQ ID NO: 11.

FIG. 7 shows non-reducing and reducing SDS-PAGE and a schematic illustration of an Fc domain formed from Fc domain monomers having the sequence of SEQ ID NO: 13.

FIG. 8 shows a non-reducing SDS-PAGE of Conjugate 3.

FIG. 9 shows a non-reducing SDS-PAGE of Conjugate 4.

FIG. 10 shows a non-reducing SDS-PAGE of Conjugate 5.

FIG. 11 shows a non-reducing SDS-PAGE of Conjugate 6.

FIG. 12 shows a non-reducing SDS-PAGE of Conjugate 7.

FIG. 13 shows a non-reducing SDS-PAGE of Conjugate 8.

FIG. 14 shows a non-reducing SDS-PAGE of Conjugate 9.

FIG. 15 shows a non-reducing SDS-PAGE of Conjugate 10.

FIG. 16 shows a non-reducing SDS-PAGE of Conjugate 11.

FIG. 17 shows a non-reducing SDS-PAGE of Conjugate 12.

FIG. 18 shows a non-reducing SDS-PAGE of Conjugate 13.

FIG. 19 shows a non-reducing SDS-PAGE of Conjugate 14.

FIG. 20 shows a non-reducing SDS-PAGE of Conjugate 15.

FIG. 21 shows a non-reducing SDS-PAGE of Conjugate 16.

FIG. 22 shows a non-reducing SDS-PAGE of Conjugate 17.

FIG. 23 shows a non-reducing SDS-PAGE of Conjugate 18.

FIG. 24 shows a non-reducing SDS-PAGE of Conjugate 19.

FIG. 25 shows a non-reducing SDS-PAGE of Conjugate 20.

FIG. 26 shows a non-reducing SDS-PAGE of Conjugate 21.

FIG. 27 shows a non-reducing SDS-PAGE of Conjugate 22.

FIG. 28 shows a non-reducing SDS-PAGE of Conjugate 23.

FIG. 29 shows a non-reducing SDS-PAGE of Conjugate 24.

FIG. 30 shows a non-reducing SDS-PAGE of Conjugate 25.

FIG. 31 shows a non-reducing SDS-PAGE of Conjugate 26.

FIGS. 32A-32C are plots showing the binding of conjugate 3 (FIG. 32A), conjugate 5 (FIG. 32B), and conjugate 7 (FIG. 32C) to bacteria as measured by flow cytometry.

FIGS. 32D-32F are images showing the binding of Conjugate 13 (FIG. 32D), Conjugate 7 (FIG. 32E), and a negative control (FIG. 32F) to E. coli, as determined by antibody binding.

FIGS. 33A-33F are graphs showing that drug conjugation to an Fc domain does not interfere with FcRn interaction. Binding to mouse FcRn is shown for conjugate 15 (FIG. 33A), conjugate 4 (FIG. 33B), conjugate 5 (FIG. 33C), and conjugate 7 (FIG. 33D). Binding to human FcRn is shown for conjugate 5 (FIG. 33E) and conjugate 7 (FIG. 33F).

FIG. 34A is a graph depicting the mean plasma concentration-time profile following administration of 10 mg/kg of conjugate 23 by intravenous (IV) or intraperitoneal (IP) administration in mice.

FIG. 34B is a table which corresponds to the graph of FIG. 34A, and which shows the individual and mean plasma concentrations and pharmacokinetics following administration of 10 mg/kg of conjugate 23 by intravenous (IV) or intraperitoneal (IP) administration in mice.

FIG. 35 is a graph that shows conjugate-mediated bacterial complement-dependent cytotoxicity (CDC).

FIG. 38 is a survival curve that shows survival data for treated and untreated mice in a lethal model of septicemia.

FIG. 37 is a survival curve that shows expanded dose ranging study in the E. coli peritonitis/septicemia model of infection.

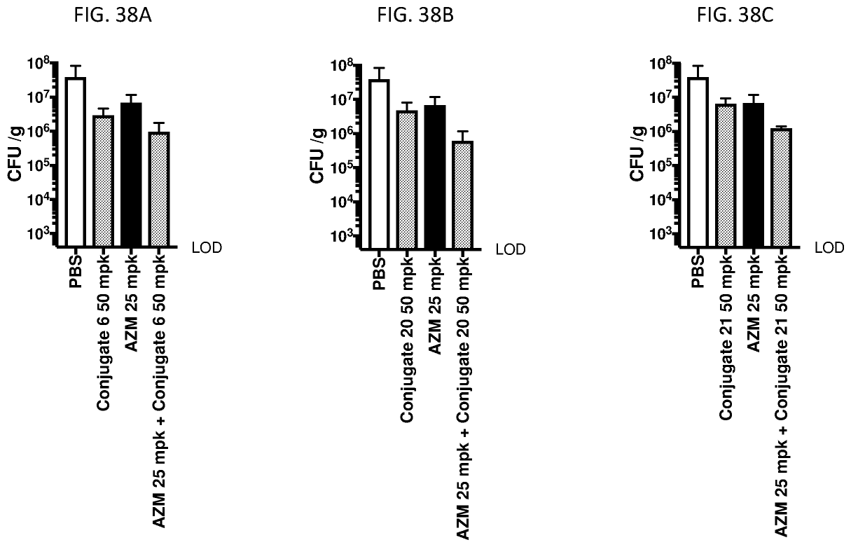

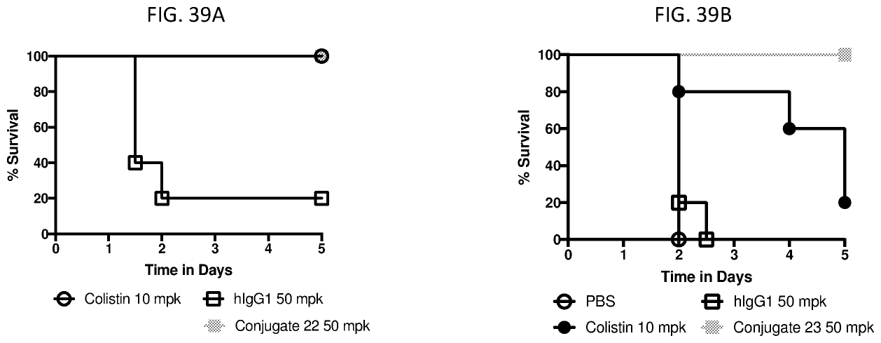

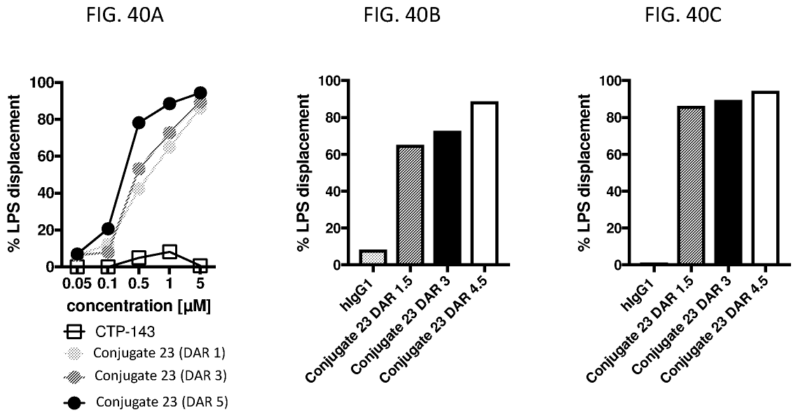

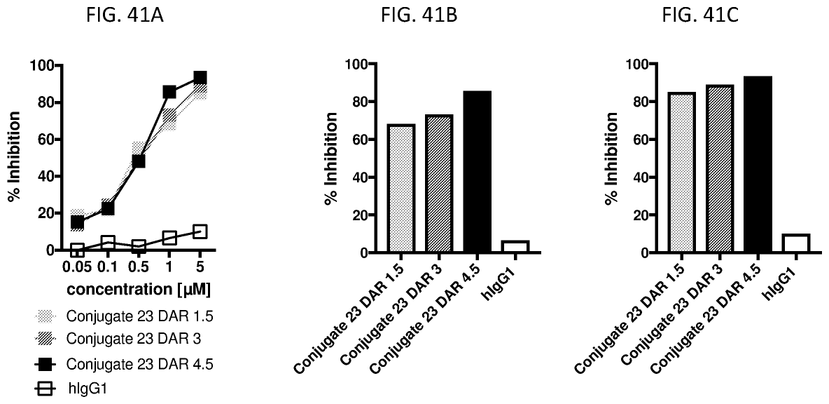

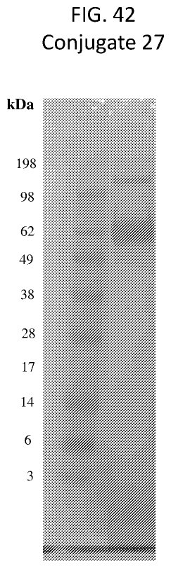

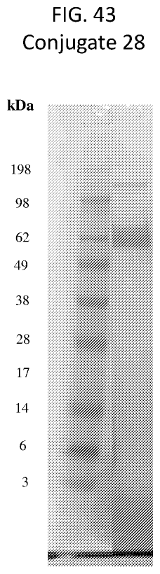

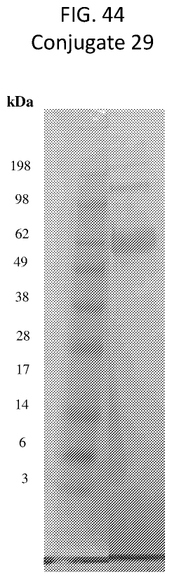

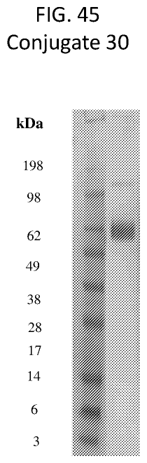

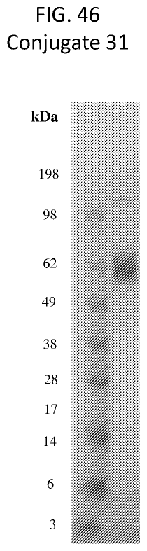

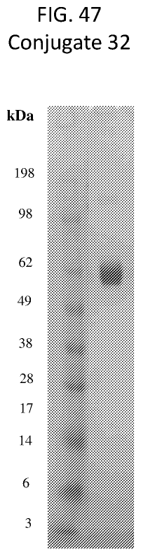

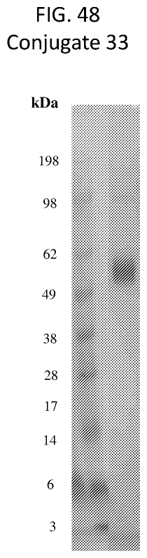

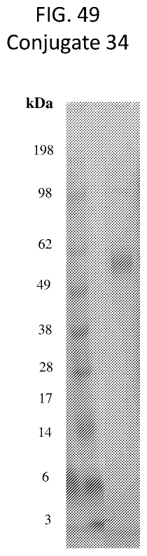

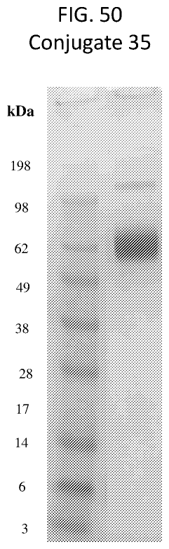

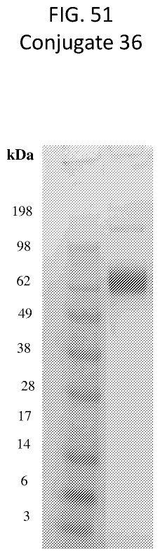

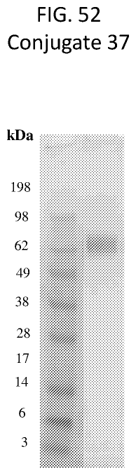

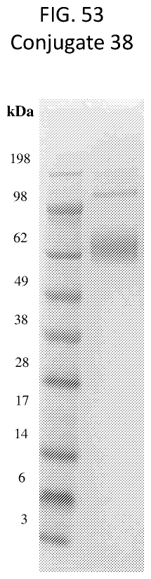

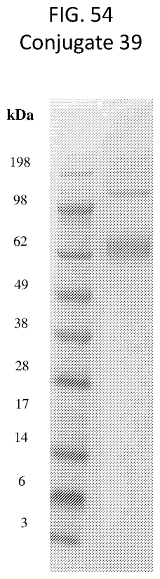

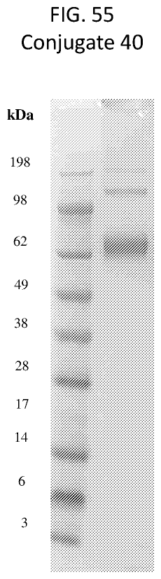

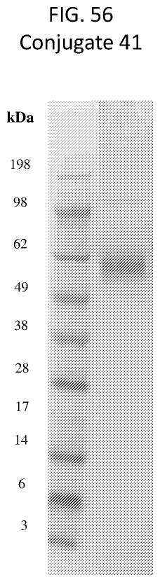

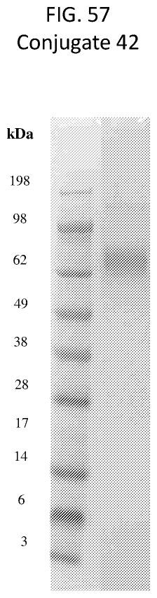

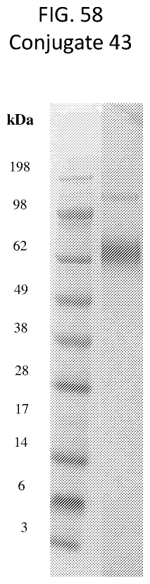

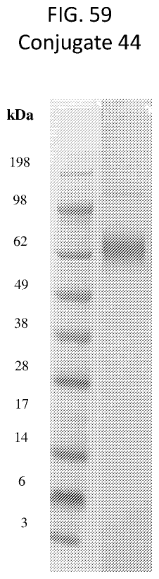

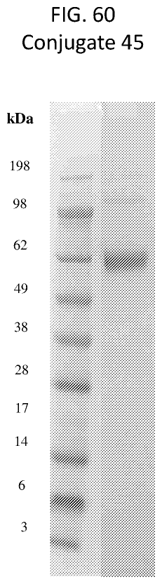

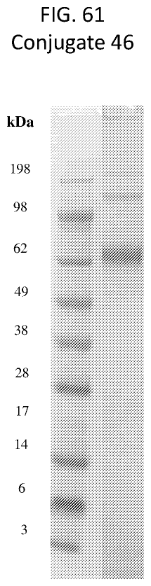

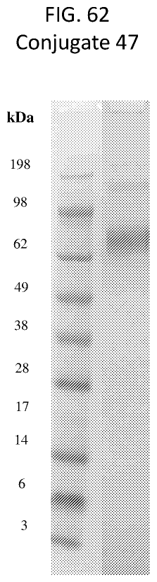

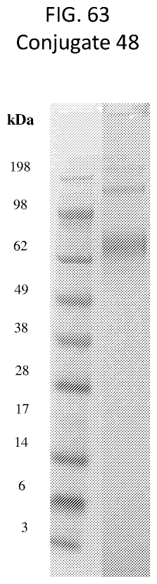

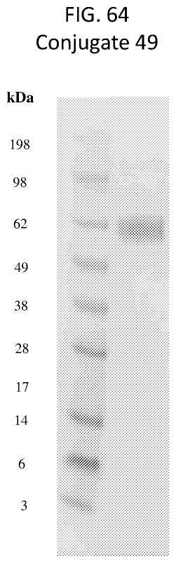

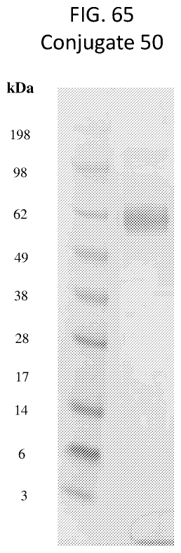

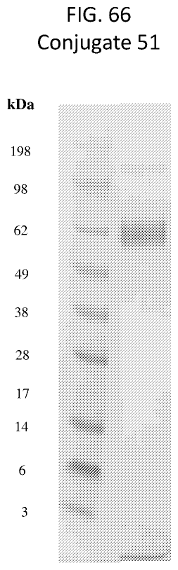

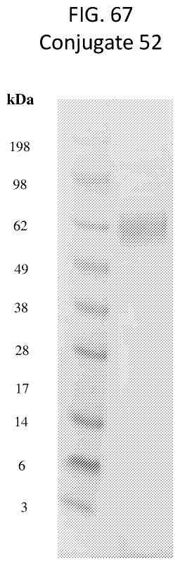

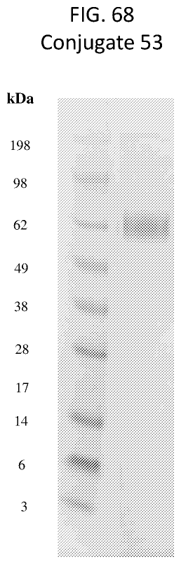

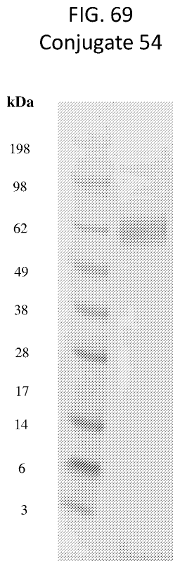

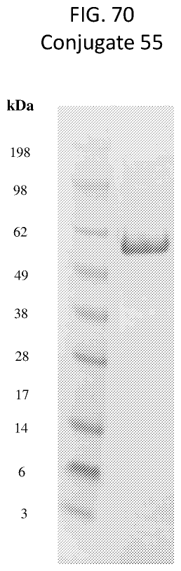

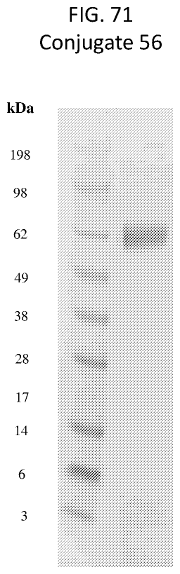

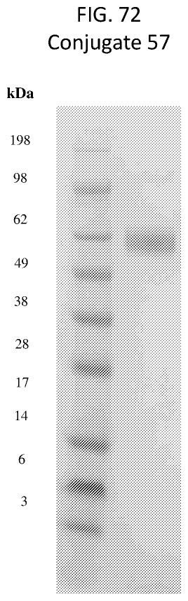

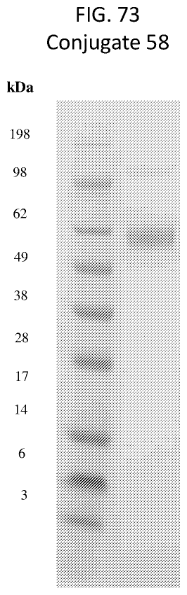

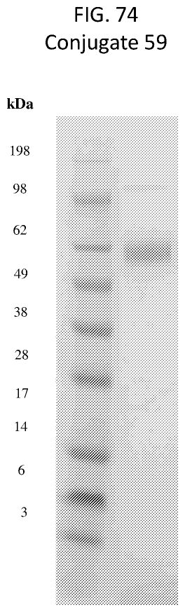

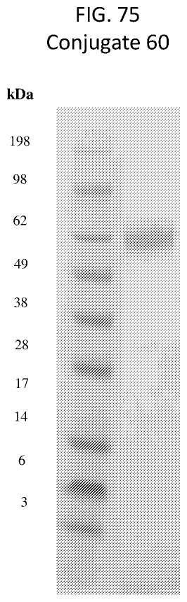

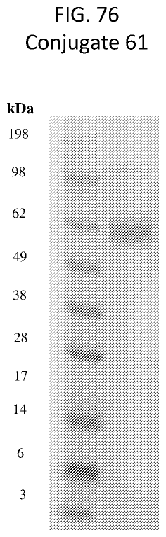

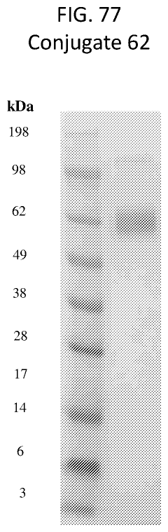

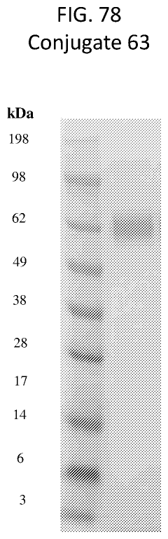

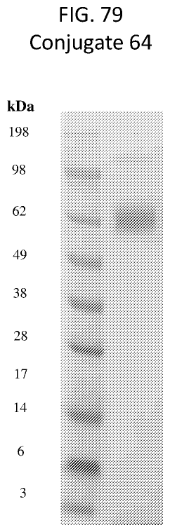

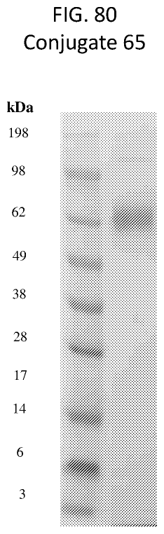

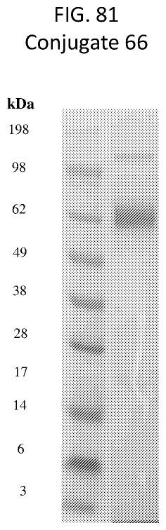

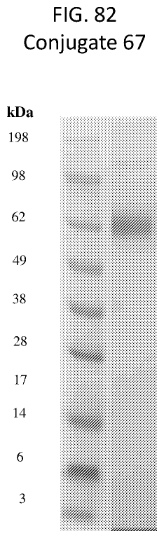

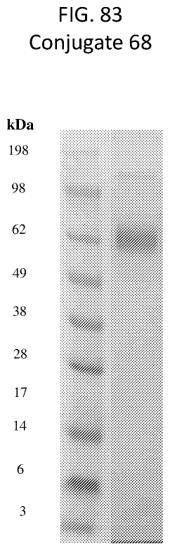

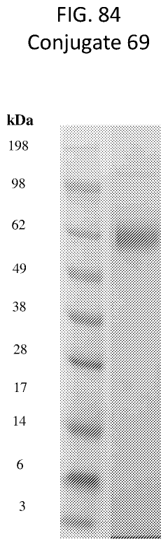

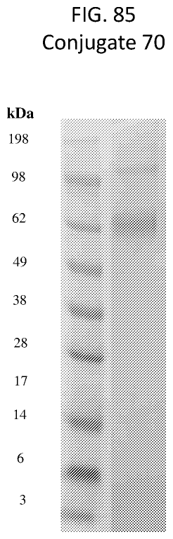

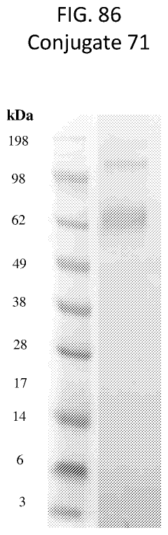

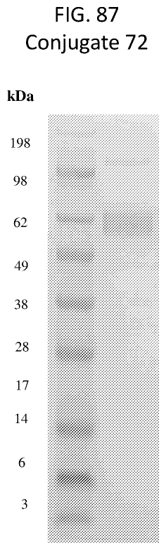

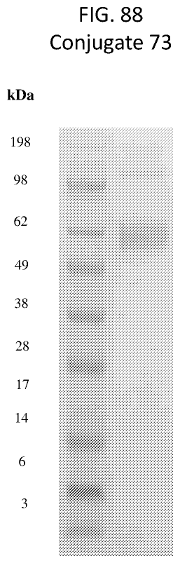

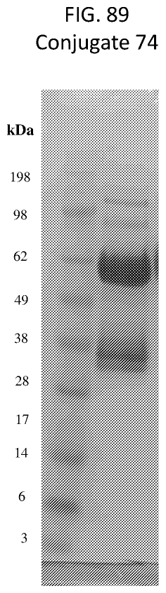

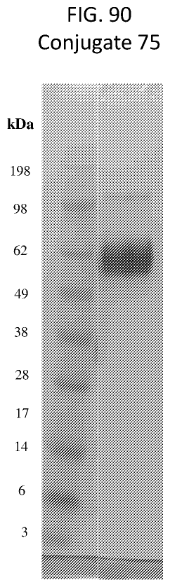

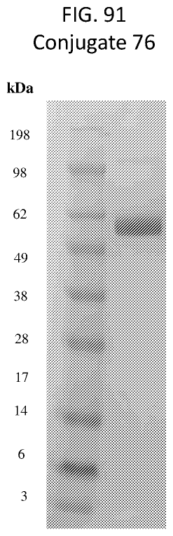

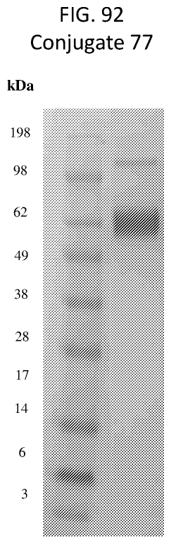

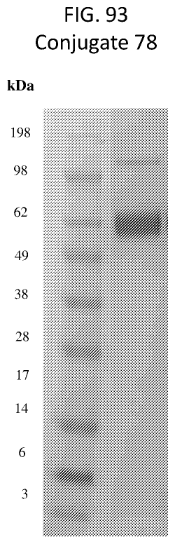

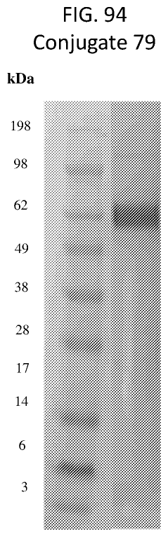

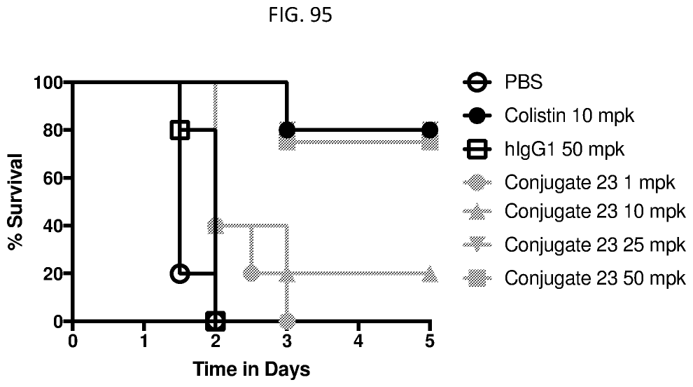

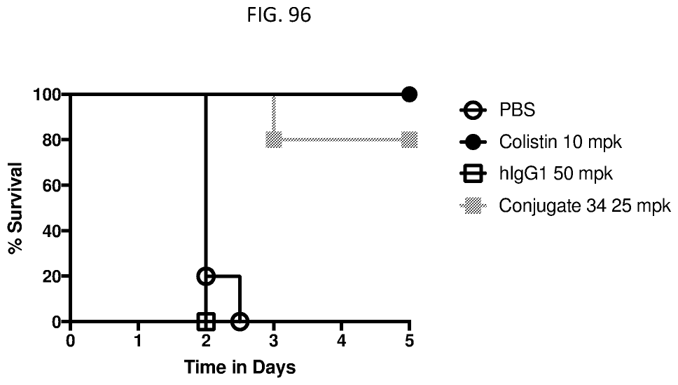

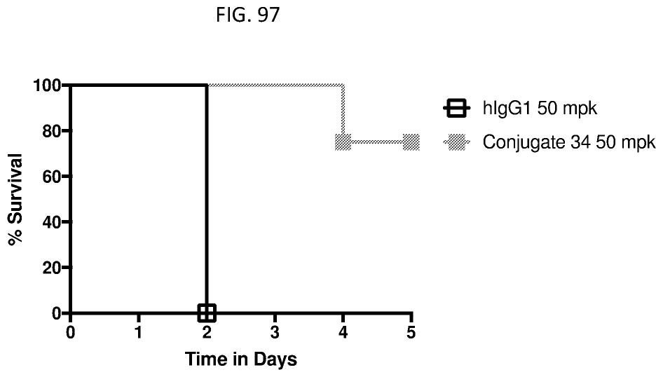

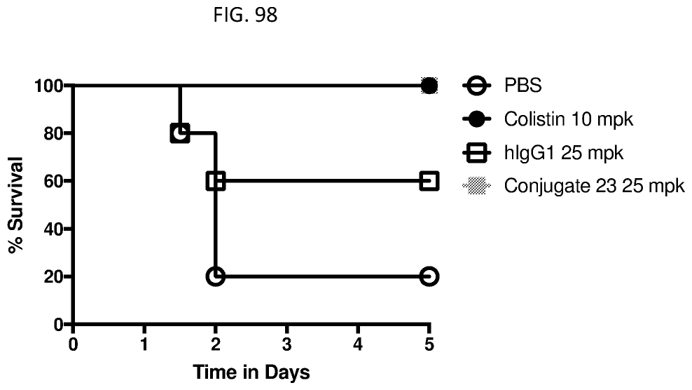

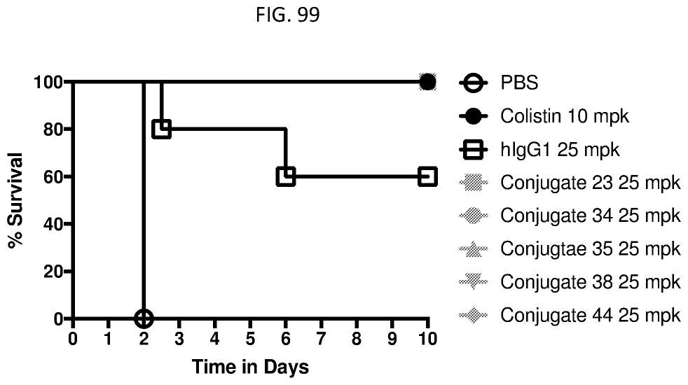

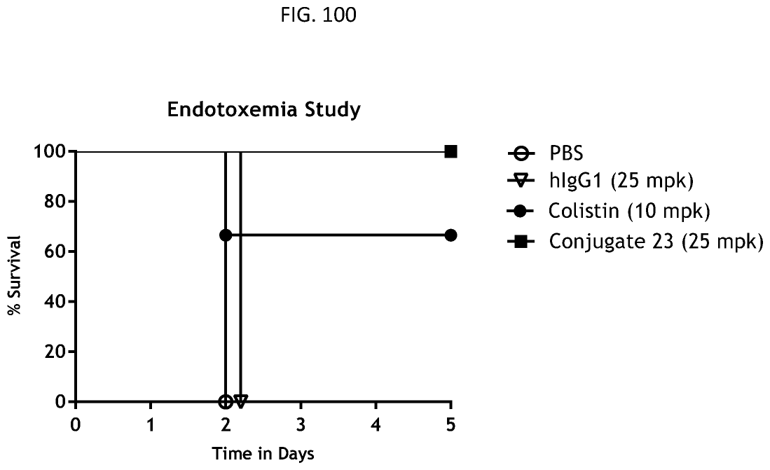

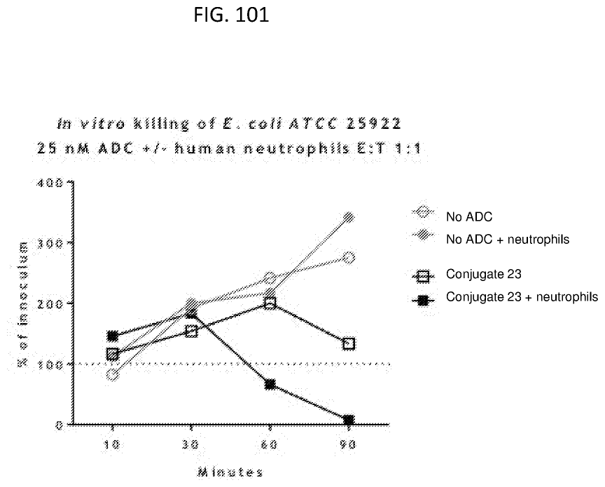

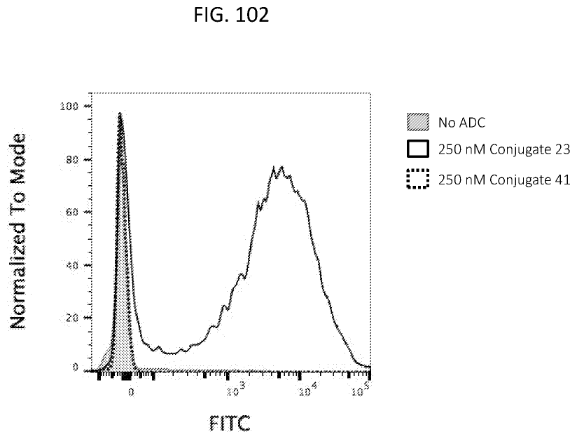

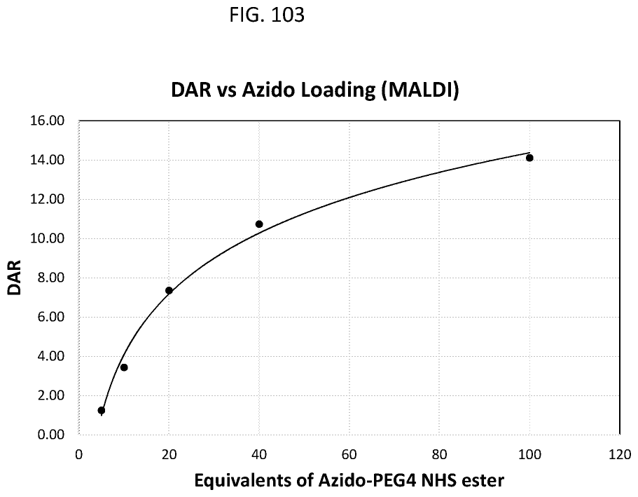

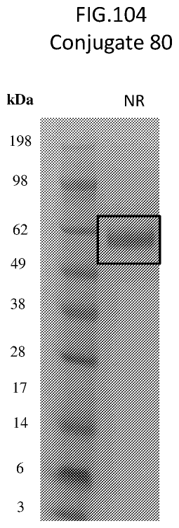

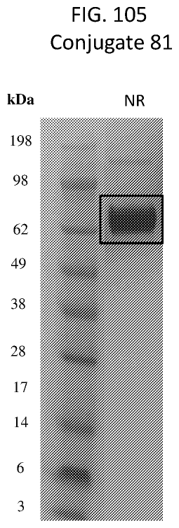

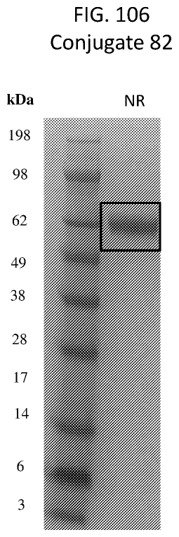

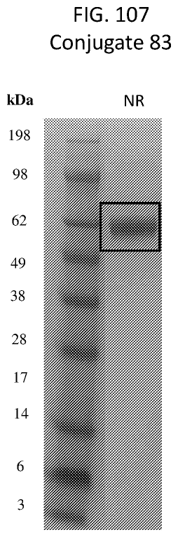

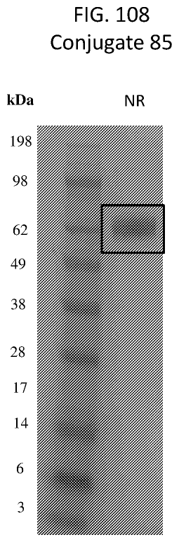

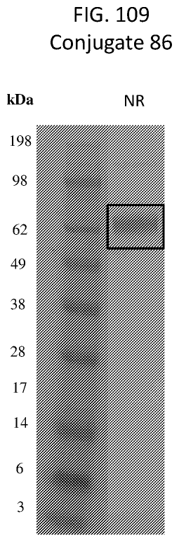

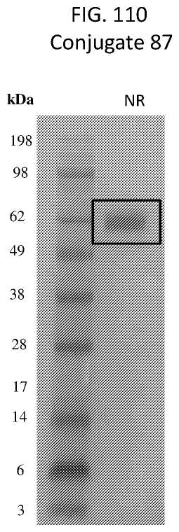

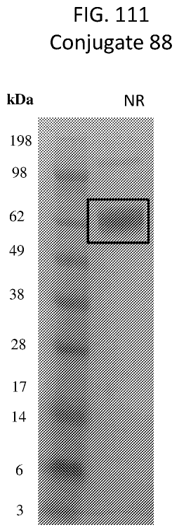

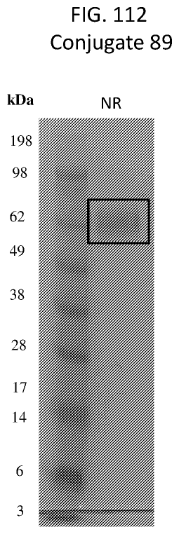

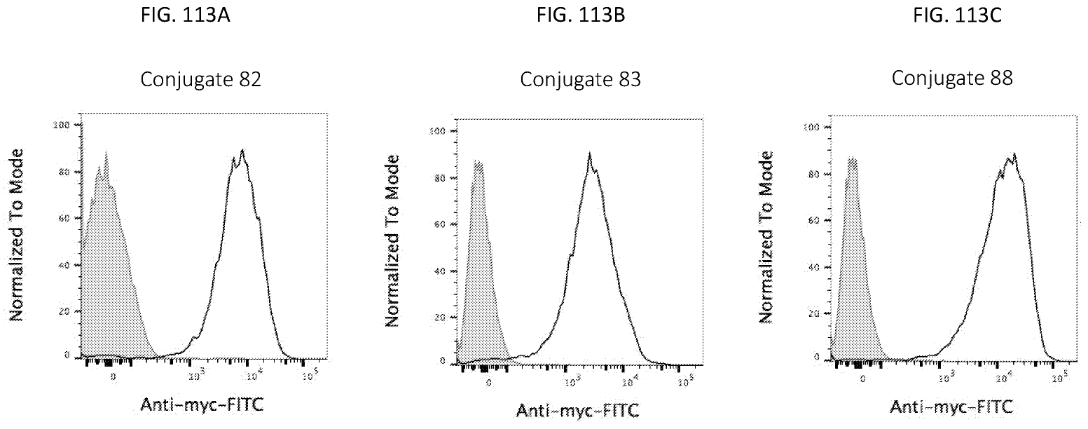

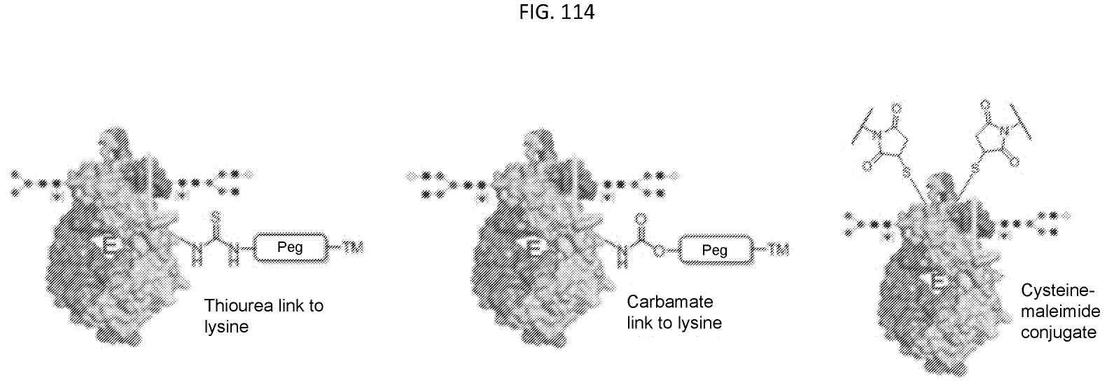

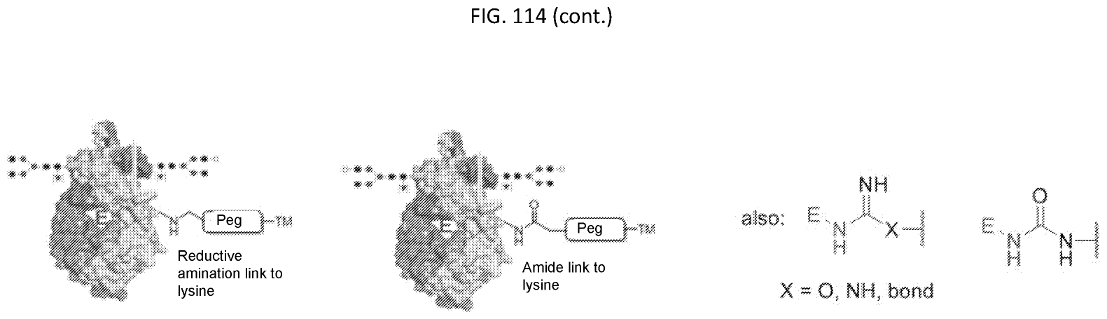

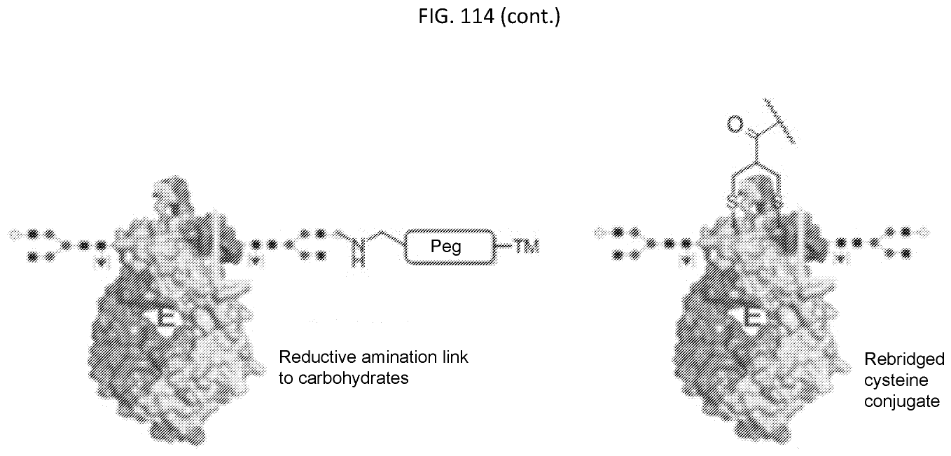

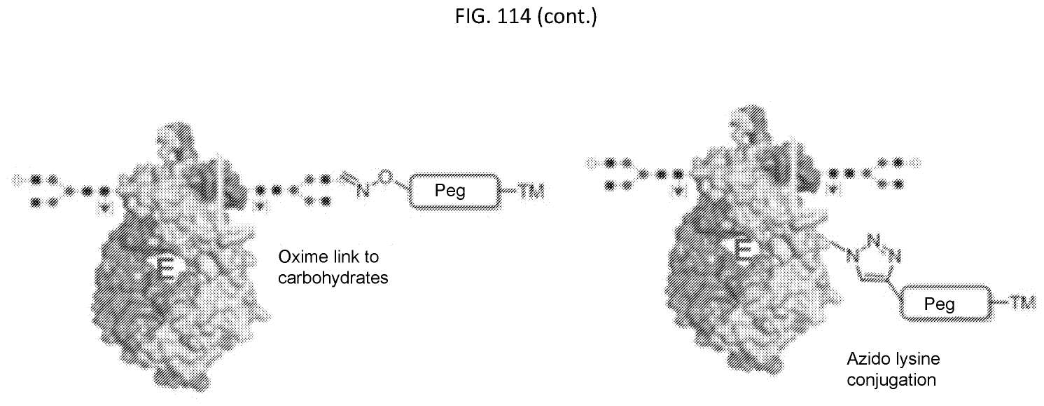

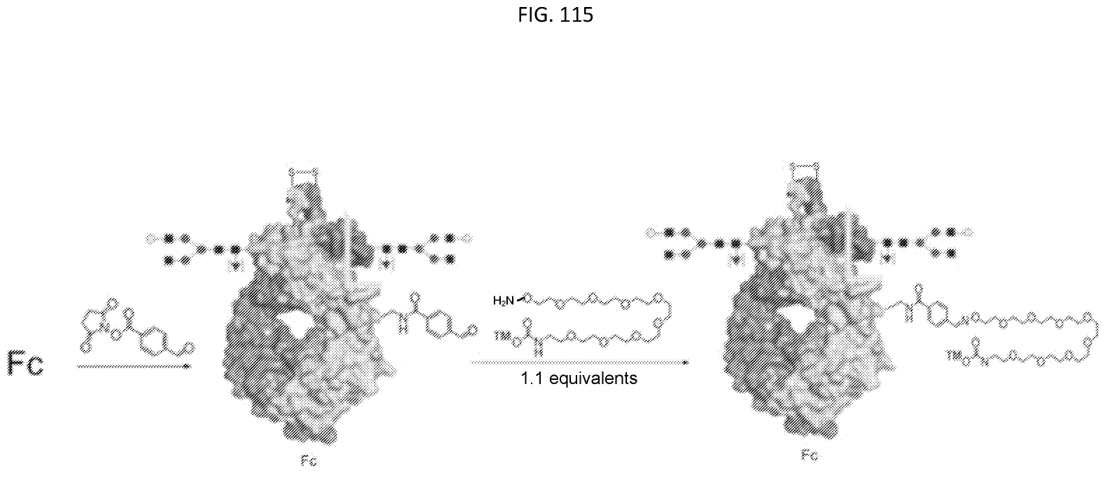

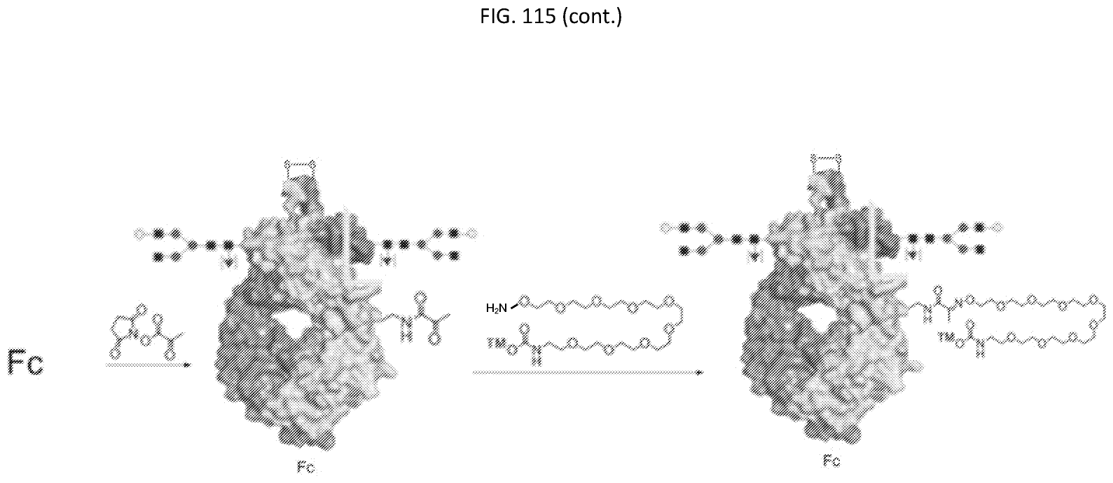

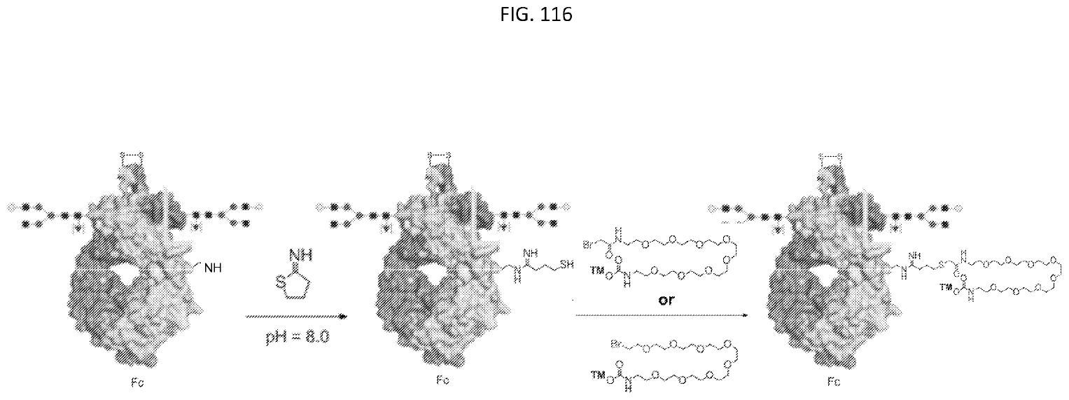

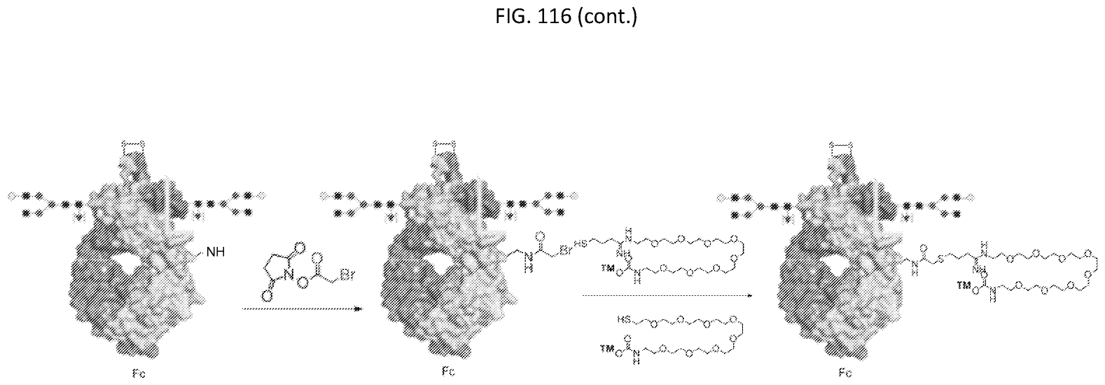

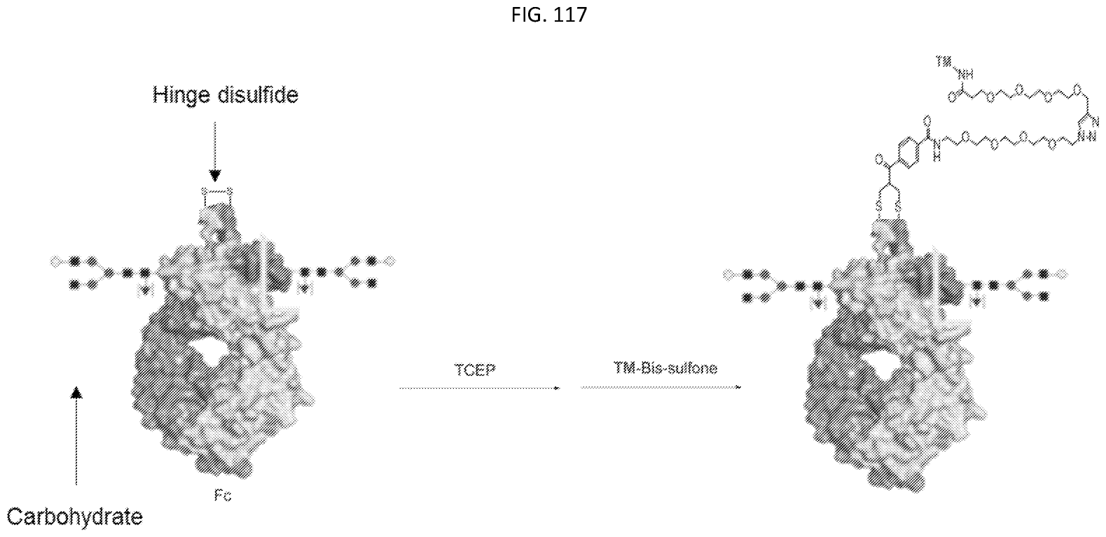

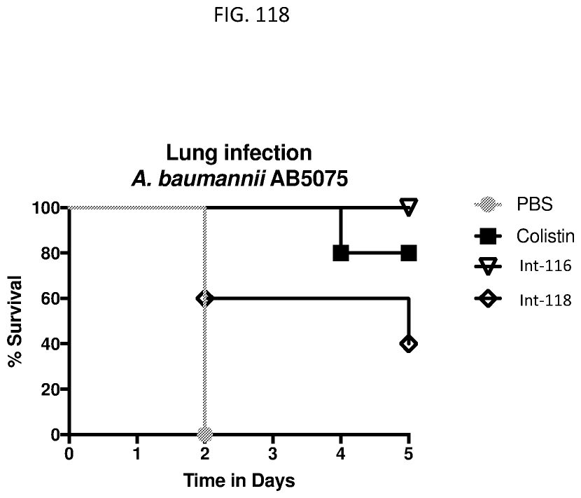

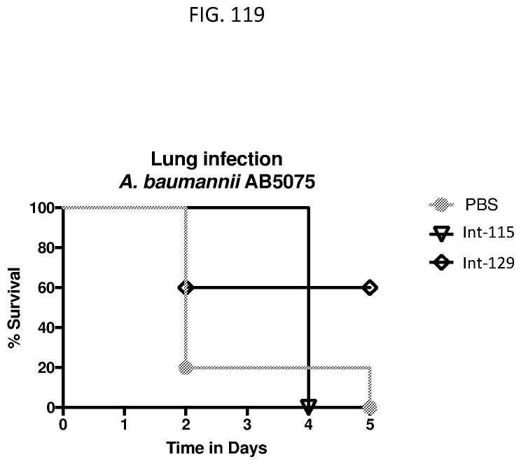

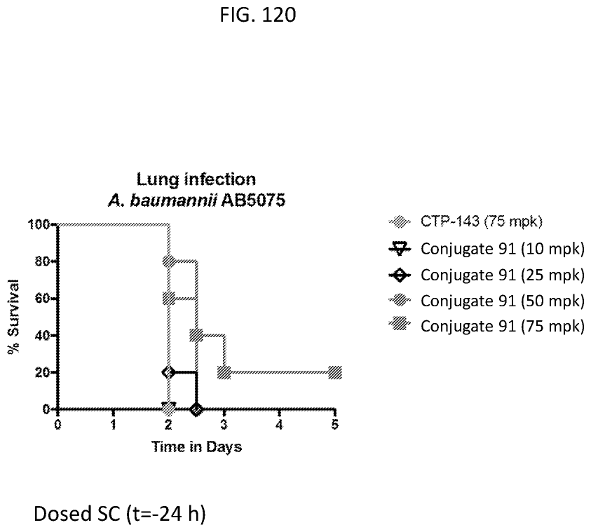

FIGS. 38A-38C are a series of graphs showing that ADC conjugates reduce CFU burden in pneumonia model against A. baumannii AB5075. Efficacy of ADC conjugates was determined upon intratracheal infection of A. baumannii AB5075 (3×10⁷ CFU) at 24 h time point. CFU burden was reduced by conjugate 6 (FIG. 38A), conjugate 20 (FIG. 38B) and conjugate 21 (FIG. 38C). Combination treatment with azithromycin (AZM) further reduced CFU burden beyond activity of AZM or conjugates alone. Conjugates (50 mpk) were administered intraperitoneally at t=−12 h and t=+1 h. AZM (25 mpk) was administered subcutaneously once at t=+1 h.

FIGS. 39A and 39B are a set of survival curves showing that ADC conjugates protect against lethal challenge in pneumonia model against A. baumannii AB5075. Efficacy of ADC conjugates was determined upon intratracheal infection with A. baumannii AB5075 (5×10⁷ CFU) over 5 days. FIG. 39A shows that conjugate 22 and colistin showed 100% protection, whereas mice treated with control IgG1 resulted in 20% survival. FIG. 39B shows that conjugate 23 resulted in 100% protection and IgG1 control was 0% survival. Colistin resulted in 20% protection at day 5. Conjugates or IgG1 (50 mpk) were administered intraperitoneally at (A) t=−12 h, t=+1 h, t=+24 h and t=+48 h or (B) t=−12 h and t=+1 h. Colistin (10 mpk) and PBS were administered intraperitoneally at (A) t=+1 h, t=+7 h, t=+24 h and t=+48 h or (B) t=+1 h, t=+5 h, t=+24 h, t=+36 h, t=+48 h and t=+60 h. Survival was monitored twice daily for 5 days.

FIGS. 40A-40C are a series of graphs showing that ADC conjugates bind purified LPS. Conjugate 23 binds to purified LPS from E. coli O111:B4 in dose-dependency from 0.05-5 μM (FIG. 40A). Correlation of DAR-dependent LPS displacement ranging from DAR 1.5 to 4.5 at 1 μM and 5 μM (FIGS. 40B and 40C, respectively). The percent LPS displacement was normalized to vehicle control.

FIGS. 41A-41C are a series of graphs showing that ADC conjugates inhibit LPS-induced nitrite oxide production by RAW macrophages. Conjugate 23 inhibit NO production by RAW macrophages in response to LPS (10 μg/mL) in dose-dependency from 0.05-5 μM at 24 h time point (FIG. 41A). Correlation of DAR-dependent inhibition ranging from DAR 1.5 to 4.5 at 1 μM and 5 μM (FIGS. 41B and 41C, respectively). The percent inhibition was normalized to vehicle control.

FIG. 42 shows a non-reducing SDS-PAGE of Conjugate 27.

FIG. 43 shows a non-reducing SDS-PAGE of Conjugate 28.

FIG. 44 shows a non-reducing SDS-PAGE of Conjugate 29.

FIG. 45 shows a non-reducing SDS-PAGE of Conjugate 30.

FIG. 48 shows a non-reducing SDS-PAGE of Conjugate 31.

FIG. 47 shows a non-reducing SDS-PAGE of Conjugate 32.

FIG. 48 shows a non-reducing SDS-PAGE of Conjugate 33.

FIG. 49 shows a non-reducing SDS-PAGE of Conjugate 34.

FIG. 50 shows a non-reducing SDS-PAGE of Conjugate 35.

FIG. 51 shows a non-reducing SDS-PAGE of Conjugate 38.

FIG. 52 shows a non-reducing SDS-PAGE of Conjugate 37.

FIG. 53 shows a non-reducing SDS-PAGE of Conjugate 38.

FIG. 54 shows a non-reducing SDS-PAGE of Conjugate 39.

FIG. 55 shows a non-reducing SDS-PAGE of Conjugate 40.

FIG. 56 shows a non-reducing SDS-PAGE of Conjugate 41.

FIG. 57 shows a non-reducing SDS-PAGE of Conjugate 42.

FIG. 58 shows a non-reducing SDS-PAGE of Conjugate 43.

FIG. 59 shows a non-reducing SDS-PAGE of Conjugate 44.

FIG. 60 shows a non-reducing SDS-PAGE of Conjugate 45.

FIG. 61 shows a non-reducing SDS-PAGE of Conjugate 46.

FIG. 62 shows a non-reducing SDS-PAGE of Conjugate 47.

FIG. 63 shows a non-reducing SDS-PAGE of Conjugate 48.

FIG. 64 shows a non-reducing SDS-PAGE of Conjugate 49.

FIG. 65 shows a non-reducing SDS-PAGE of Conjugate 50.

FIG. 66 shows a non-reducing SDS-PAGE of Conjugate 51.

FIG. 67 shows a non-reducing SDS-PAGE of Conjugate 52.

FIG. 68 shows a non-reducing SDS-PAGE of Conjugate 53.

FIG. 69 shows a non-reducing SDS-PAGE of Conjugate 54.

FIG. 70 shows a non-reducing SDS-PAGE of Conjugate 55.

FIG. 71 shows a non-reducing SDS-PAGE of Conjugate 56.

FIG. 72 shows a non-reducing SDS-PAGE of Conjugate 57.

FIG. 73 shows a non-reducing SDS-PAGE of Conjugate 58.

FIG. 74 shows a non-reducing SDS-PAGE of Conjugate 59.

FIG. 75 shows a non-reducing SDS-PAGE of Conjugate 60.

FIG. 76 shows a non-reducing SDS-PAGE of Conjugate 61.

FIG. 77 shows a non-reducing SDS-PAGE of Conjugate 62.

FIG. 78 shows a non-reducing SDS-PAGE of Conjugate 63.

FIG. 79 shows a non-reducing SDS-PAGE of Conjugate 64.

FIG. 80 shows a non-reducing SDS-PAGE of Conjugate 65.

FIG. 81 shows a non-reducing SDS-PAGE of Conjugate 66.

FIG. 82 shows a non-reducing SDS-PAGE of Conjugate 67.

FIG. 83 shows a non-reducing SDS-PAGE of Conjugate 68.

FIG. 84 shows a non-reducing SDS-PAGE of Conjugate 69.

FIG. 85 shows a non-reducing SDS-PAGE of Conjugate 70.

FIG. 86 shows a non-reducing SDS-PAGE of Conjugate 71.

FIG. 87 shows a non-reducing SDS-PAGE of Conjugate 72.

FIG. 88 shows a non-reducing SDS-PAGE of Conjugate 73.

FIG. 89 shows a non-reducing SDS-PAGE of Conjugate 74.

FIG. 90 shows a non-reducing SDS-PAGE of Conjugate 75.

FIG. 91 shows a non-reducing SDS-PAGE of Conjugate 76.

FIG. 92 shows a non-reducing SDS-PAGE of Conjugate 77.

FIG. 93 shows a non-reducing SDS-PAGE of Conjugate 78.

FIG. 94 shows a non-reducing SDS-PAGE of Conjugate 79.

FIG. 95 is a survival curve showing the efficacy of ADC conjugate 23 in mouse pneumonia model vs. AB5075. Survival of mice (n=5 per group) was monitored for 5 days. ADC conjugate 23 showed a dose-dependent protection: 0% protection at 1 mpk, 20% protection at 10 mpk and 80% protection at 25 and 50 mpk. Colistin used as positive control resulting in 80% protection. In comparison, control IgG1 and PBS were not protective and mice succumbed to infection between day 1.5-2.

FIG. 96 is a survival curve showing the efficacy of ADC conjugate 34 in mouse pneumonia model vs. PAO1. Survival of mice (n=5 per group) was monitored for 5 days. A single dose of ADC conjugate 34 was protective (80% survival) against lethal challenge with P. aeruginosa PAO1. Colistin used as positive control resulting in complete protection. In comparison, control IgG1 and PBS were not protective and mice succumbed to infection between day 2-2.5.

FIG. 97 is a survival curve showing the efficacy of ADC conjugate 34 in mouse pneumonia model vs. AB5075. Survival of mice (n=4 per group) was monitored for 5 days. A single dose of ADC conjugate 34 was protective (75% survival) against lethal challenge with A. baumannii AB5075. Control IgG1 was not protective and mice succumbed to infection on day 2.

FIG. 98 is a survival curve showing the efficacy of ADC conjugate 23 in mouse pneumonia model vs. PAO1. Survival of mice (n=5 per group) was monitored for 5 days. A single dose of ADC conjugate 23 was completely protective against challenge with P. aeruginosa PAO1. Positive control, colistin, was was also protective (100%). Controls IgG1 (60% survival) and PBS (20% survival) were only partially protective.

FIG. 99 is a survival curve showing the efficacy of ADC conjugate 23 in mouse pneumonia model vs. PAO1. Survival of mice (n=5 per group) was monitored for 5 days. A single dose of ADC conjugates 23, 34, 35, 38 or 44 was completely protective against challenge with P. aeruginosa PAO1. Positive control, colistin, was also protective (100%). Controls IgG1 (60% survival) was only partially protective and mice in PBS group succumbed to infection on day 2.

FIG. 100 is a survival curve showing the protective effect of Conjugate 23 against endotoxemia. Mice dosed with the vehicle or hIgG1 reached 100% mortality within 48 hours of LPS challenge. Mice receiving multiple doses of colistin were partially protected with ˜30% of animals reaching death after 5 days. In contrast, all mice treated with conjugate 23 were rescued with 0% mortality.

FIG. 101 is a graph showing the in vitro killing of E. coli ATCC 25922 following treatment with either conjugate 23 alone, or conjugate 23 and purified human neutrophils.

FIG. 102 is a FACS plot showing the neutrophil phagocytosis of FITC-labeled E. coli (K-12) BioParticles opsonized with conjugate 23, conjugate 41, or media alone.

FIG. 103 shows a graph of the DAR values of the azido-hIgG1(Myc)Fc that were measured by MALDI-TOF at varying equivalents of azido-PEG4-NHS ester.

FIG. 104 shows a non-reducing SDS-PAGE of Conjugate 80.

FIG. 105 shows a non-reducing SDS-PAGE of Conjugate 81.

FIG. 106 shows a non-reducing SDS-PAGE of Conjugate 82.

FIG. 107 shows a non-reducing SDS-PAGE of Conjugate 83.

FIG. 108 shows a non-reducing SDS-PAGE of Conjugate 85.

FIG. 109 shows a non-reducing SDS-PAGE of Conjugate 86.

FIG. 110 shows a non-reducing SDS-PAGE of Conjugate 87.

FIG. 111 shows a non-reducing SDS-PAGE of Conjugate 88.

FIG. 112 shows a non-reducing SDS-PAGE of Conjugate 89.

FIGS. 113A-113C are plots showing the binding of conjugate 82 (FIG. 113A), conjugate 83 (FIG. 113B), and conjugate 88 (FIG. 113C) to bacteria as measured by flow cytometry.

FIG. 114 is an image depicting exemplary methods of conjugating a polymyxin monomer or dimer, e.g., via a linker, to an Fc domain monomer or an Fc domain.

FIG. 115 is an image depicting a method of conjugating a polymyxin monomer or dimer, e.g., via a linker, to an Fc domain monomer or an Fc domain by oxime conjugation to an amino acid residue, e.g., a nitrogen atom of a surface exposed lysine.

FIG. 116 is an image depicting a method of conjugating a polymyxin monomer or dimer, e.g., via a linker, to an Fc domain monomer or an Fc domain by thioether conjugation to an amino acid residue, e.g., a nitrogen atom of a surface exposed lysine.

FIG. 117 is an image depicting a method of conjugating a polymyxin monomer or dimer, e.g., via a linker, to an Fc domain monomer or an Fc domain by rebridged cysteine conjugation, e.g., rebridged cysteine conjugation to a pair of sulfur atoms of two hinge cysteines in an Fc domain monomer or Fc domain.

FIG. 118 is a survival curve showing the efficacy of Int-116 and Int-118 (5 mg/kg, twice intravenously, t=+1 h and +4 h) in mouse pneumonia model vs. AB5075. Survival of mice (n=5 per group) was monitored for 5 days. Colistin and Int-118 were partially protective (80% and 40% respectively). In contrast, Int-116 was protective (100%) against lethal challenge with A. baumannii AB5075. Control PBS was not protective and mice succumbed to infection on day 2.

FIG. 119 is a survival curve showing the efficacy of Int-115 and Int-129 (5 mg/kg, twice intravenously, t=+1 h and +4 h) in mouse pneumonia model vs. AB5075. Survival of mice (n=5 per group) was monitored for 5 days. Int-129 was protective showing 60% survival. Int-115 was able to delay death of all mice until day 4 post-infection. All mice treated with PBS as control succumbed to infection at day 5. The positive control, colistin was protective (80%).

FIG. 120 is a survival curve showing the efficacy of ADC conjugate 91 in mouse pneumonia model vs. AB5075. Survival of mice (n=5 per group) was monitored for 5 days. ADC conjugate 91 at 75 mg/kg showed 20% protection at day 5 post-infection, whereas lower concentration or CTP-143 control showed no protection. At 50 mg/kg of Conjugate 91 a delay in death of 0.5 days was observed.

### DETAILED DESCRIPTION

The disclosure features conjugates, compositions, and methods for the treatment of bacterial infections (e.g., Gram-negative bacterial infections). The conjugates disclosed herein include monomers or dimers of cyclic heptapeptides (e.g., two polymyxin cores) conjugated to Fc domains. The dimers of cyclic heptapeptides are linked to each other through a linker and/or one or two peptides (e.g., a peptide including a 1-5 amino acid residue(s)). The monomers or dimers of cyclic heptapeptides in the conjugates described herein bind to lipopolysaccharides (LPS) in the cell membrane of Gram-negative bacteria to disrupt and permeabilize the cell membrane, leading to cell death and/or sensitization of the Gram-negative bacteria to other antibiotics. The Fc domains in the conjugates bind to FcγRs (e.g., FcRn, FcγRI, FcγRIIa, FcγRIIc, FcγRIIIa, and FcγRIIIb) on immune cells, e.g., neutrophils, to activate phagocytosis and effector functions, such as antibody-dependent cell-mediated cytotoxicity (ADCC), thus leading to the engulfment and destruction of bacterial cells by immune cells and further enhancing the antibacterial activity of the conjugates.

### I. Bacterial Infections

Pathogenic bacteria cause bacterial infections and diseases such as tuberculosis, pneumonia, and foodborne illnesses. Bacteria may be categorized into two major types: Gram-positive bacteria and Gram-negative bacteria. Gram-positive bacteria possess a thick cell wall containing multiple layers of peptidoglycan and teichoic acids, while Gram-negative bacteria have a relatively thin cell wall containing fewer layers of peptidoglycan that are surrounded by a second lipid membrane containing lipopolysaccharides (LPS) and lipoproteins. LPS, also called endotoxins, are composed of polysaccharides and lipid A. These differences in bacterial cell wall structure can produce differences in antibiotic susceptibility. Examples of Gram-positive bacteria include, but are not limited to, bacteria in the genus Streptococcus (e.g., Streptococcus pyogenes), bacteria in the genus Staphylococcus (e.g., Staphylococcus cohnii), bacteria in the genus Corynebacterium (e.g., Corynebacterium auris), bacteria in the genus Listeria (e.g., Listeria grayi), bacteria in the genus Bacillus (e.g., Bacillus aerius), and bacteria in the genus Clostridium (e.g., Clostridium acetium). Examples of Gram-negative bacteria include, but are not limited to, bacteria in the genus Escherichia (e.g., Escherichia coli), bacteria in the genus Klebsiella (e.g., Klebsiella granulomatis, Klebsiella oxytoca, Klebsiella pneumoniae, Klebsiella terrigena, and Klebsiella variicola), bacteria in the genus Acinetobacter (e.g., Acinetobacter baumannii, Acinetobacter calcoaceticus, Acinetobacter kookii, and Acinetobacter junii), bacteria in the genus Pseudomonas (e.g., Pseudomonas aeruginosa), bacteria in the genus Neisseria (e.g., Neisseria gonorrhoeae), bacteria in the genus Yersinia (e.g., Yersinia pestis), bacteria in the genus Vibrio (e.g., Vibrio cholerae), bacteria in the genus Campylobacter (e.g., Campylobacter jejuni), and bacteria in the genus Salmonella (e.g., Salmonella enterica).

Bacteria may evolve to become more or fully resistant to antibiotics. Resistance may arise through natural resistance in certain types of bacteria, spontaneous random genetic mutations, and/or by inter- or intra-species horizontal transfer of resistance genes. Resistant bacteria are increasingly difficult to treat, requiring alternative medications or higher doses, which may be more costly or more toxic. Bacteria resistant to multiple antibiotics are referred to as multidrug resistant (MDR) bacteria. For example, the mcr-1 gene encodes a phosphoethanolamine transferase (MCR-1) which confers resistance to colistin, a natural polymyxin, through modification of LPS. This is the first known horizontally-transferable resistance determinant for the polymyxin class of antibiotics. The mcr-1 gene has also been found in bacterial strains which already possess resistance to other classes of antibiotics, such as in carbapenem-resistant Enterobacteriaceae (CRE). An mcr-1 resistance plasmid refers to a bacterial plasmid that carries mcr-1 alone or in combination with other antibiotic resistance genes. A mcr-1 resistance plasmid refers to a bacterial plasmid that carries one or more antibiotic resistance genes. Examples of mcr-1 resistance plasmids include, but are not limited to, pHNSHP45, pMR0516mcr, pESTMCR, pAF48, pAF23, pmcr1-lncX4, pmcr1-lncl2, pA31-12, pVT553, pICBEC72Hmcr, pE15004, pE15015, and pE15017.

In another example, the mcr-2 gene also confers resistance to colistin. The mcr-2 gene was identified in porcine and bovine colistin-resistance E. coli that did not contain mcr-1 (Xavier et al., Euro Surveill 21(27), 2016). The mcr-2 gene is a 1,617 bp phspoethanolamine transferase harbored on an IncX4 plasmid. The mcr-2 gene has 76.7% nucleotide identity to mcr-1. Analysis of mcr-2 harboring plasmids from E. coli isolates shows that the mobile element harboring mcr-2 is an IS element of the IS₁₅₉₅ superfamily, which are distinguished by the presence of an ISXO₂-like transposase domain (Xavier et al., supra). The MCR-2 protein was predicted to have two domains, with domain 1 (1-229 residues) as a transporter and domain 2 (230-538 residues) as a transferase domain. An mcr-2 resistance plasmid refers to a bacterial plasmid that carries mcr-2 alone or in combination with other antibiotic resistance genes. A mcr-2 resistance plasmid refers to a bacterial plasmid that carries one or more antibiotic resistance genes. Mcr-2 resistance plasmids include, but are not limited to, pKP37-BE and pmcr2-IncX4. In another example, the mcr-3, mcr-4, mcr-5, mcr-6, mcr-7, and mcr-8 genes also confer resistance to colistin, described in Wang et al., Emerging Microbes & infections 7:122 (2018), which is herein incorporated by reference in its entirety.

Furthermore, resistant strain E. coli BAA-2489 possesses the New Delhi metallo-p-lactamase (NDM-1) enzyme, which makes bacteria resistant to a broad range of p-lactam antibiotics. Additionally, E. coli BAA-2469 is also known to be resistant to penicillins (e.g., ticarcillin, ticarcillin/clavulanic acid, piperacillin, ampicillin, and ampicillin/sulbactam), cephalosporins (e.g., cefalotin, cefuroxime, cefuroxime, cefotetan, cefpodoxime, cefotaxime, ceftizoxime, cefazolin, cefoxitin, ceftazidime, ceftriaxone, and cefepime), carbapenems (e.g., doripenem, meropenem, ertapenem, imipenem), quinolones (e.g., nalidixic acid, moxifloxacin, norfloxacin, ciprofloxacin, and levofloxacin), aminoglycosides (e.g., amikacin, gentamicin, and tobramycin), and other antibiotics (e.g., tetracycline, tigecycline, nitrofurantoin, aztreonam, trimethoprim/sulfamethoxazole).

In some embodiments, a resistant strain of bacteria possesses the mcr-1 gene, the mcr-2 gene, the mcr-3 gene, the mcr-4 gene, the mcr-5 gene, the mcr-8 gene, the mcr-7 gene, the mcr-8 gene, and/or a chromosomal mutation conferring polymyxin resistance. In some embodiments, a resistant strain of bacteria is a resistant strain of E. coli (e.g., E. coli BAA-2489).

### II. Conjugates of the Disclosure

Provided herein are synthetic conjugates useful in the treatment of bacterial infections (e.g., Gram-negative bacterial infections). The conjugates disclosed herein include an Fc domain conjugated to one or more monomers of cyclic heptapeptides or one or more dimers of two cyclic heptapeptides. The dimers of two cyclic heptapeptides include a first cyclic heptapeptide (e.g., a first polymyxin core) and a second cyclic heptapeptide (e.g., a second polymyxin core). The first and second cyclic heptapeptides are linked to each other by way of a linker and/or one or two peptides (e.g., each peptide including a 1-5 amino acid residue(s)).

Without being bound by theory, in some aspects, conjugates described herein bind to the cell membrane of Gram-negative bacteria (e.g., bind to LPS in the cell membrane of Gram-negative bacteria) through the interactions between the cyclic heptapeptides in the conjugates and the cell membrane of Gram-negative bacteria. The binding of the conjugates to the cell membrane of Gram-negative bacteria disrupt and permeabilize the cell membrane, leading to cell death and/or sensitization of the Gram-negative bacteria to other antibiotics. In some embodiments, the initial association of the conjugates with the bacterial cell membrane occurs through electrostatic interactions between the cyclic heptapeptides in the conjugates and the anionic LPS in the outer membrane of Gram-negative bacteria, disrupting the arrangement of the cell membrane. Specifically, conjugates described herein may bind to lipid A in the LPS. More specifically, the cyclic heptapeptides in the conjugates described herein may bind to one or both phosphate groups in lipid A. In some embodiments, antibiotic-resistant bacteria (e.g., antibiotic-resistant, Gram-negative bacteria) has one phosphate group in lipid A. In some embodiments, conjugates described herein may bind to multiple Gram-negative bacterial cells at the same time. The binding of the conjugates described herein to the LPS may also displace Mg²⁺ and Ca²⁺ cations that bridge adjacent LPS molecules, causing, e.g., membrane permeabilization, leakage of cellular molecules, inhibition of cellular respiration, and/or cell death. Furthermore, in addition to disrupting the cell membrane of Gram-negative bacteria, the Fc domain in the conjugates described herein binds to the FcγRs (e.g., FcRn, FcγRI, FcγRIIa, FcγRIIc, FcγRIIIa, and FcγRIIIb) on immune cells and serves as a gradient against which immune cells chemotax to the site of bacterial infection and/or growth. The binding of the Fc domain in the conjugates described herein to the FcγRs on immune cells activates phagocytosis and effector functions, such as antibody-dependent cell-mediated cytotoxicity (ADCC), thus leading to the engulfment and destruction of Gram-negative bacterial cells by immune cells and further enhancing the antibacterial activity of the conjugates.

Conjugates provided herein are described by any one of formulas (I)-(XXXVI). In some embodiments, the conjugates described herein include one or more monomers of cyclic heptapeptides conjugated to an Fc domain. In some embodiments, the conjugates described herein include one or more dimers of cyclic heptapeptides conjugated to an Fc domain. In some embodiments, when n is 2. E (an Fc domain monomer) dimerizes to form an Fc domain. Conjugates described herein may be synthesized using available chemical synthesis techniques in the art. In some embodiments, available functional groups in the cyclic heptapeptides and the linker, e.g., amines, carboxylic acids, maleimides, bis-sulfones, azides, alkynes, and/or hydroxyl groups, may be used in making the conjugates described herein. For example, the linking nitrogen (described further herein) in a cyclic heptapeptide may form an amide bond with the carbon in a carboxylic acid group in the linker. A peptide including one or more (e.g., 1-3; 1, 2, or 3) amino acid residues (e.g., natural and/or non-natural amino acid residues) may be also be covalently attached to the linking nitrogen of the cyclic heptapeptide through forming an amide bond between the carbon in a carboxylic acid group in the peptide and the linking nitrogen. In cases where a functional group is not available for conjugation, a molecule may be derivatized using conventional chemical synthesis techniques that are well known in the art. In some embodiments, the conjugates described herein contain one or more chiral centers. The conjugates include each of the isolated stereoisomeric forms as well as mixtures of stereoisomers in varying degrees of chiral purity, including racemic mixtures. It also encompasses the various diastereomers, enantiomers, and tautomers that can be formed.

Cyclic Heptapeptide or Polymyxin Core

In some aspects, a cyclic heptapeptide or polymyxin core, as used herein, refers to certain compounds that bind to lipopolysaccharides (LPS) in the cell membrane of Gram-negative bacteria to disrupt and permeabilize the cell membrane, leading to cell death and/or sensitization to other antibiotics. In some aspects, cyclic heptapeptide, as used herein, refers to certain compounds that kill or inhibit the growth of Gram-negative bacteria as determined by measuring the minimum inhibitory concentration (MIC) against at least one Gram-negative bacteria as known in the art, wherein the MIC is 128 μg/mL or less.

Cyclic heptapeptides are composed of, at least, amino acid residues, each of which may, independently, may have a D- or L-configuration, assembled as a cyclic heptapeptide ring. A cyclic heptapeptide includes seven natural or non-natural amino acid residues attached to each other in a closed ring. The ring contains six bonds formed by linking the carbon in the α-carboxyl group of one amino acid residue to the nitrogen in the α-amino group of the adjacent amino acid residue and one bond formed by linking the carbon in the α-carboxyl group of one amino acid residue to the nitrogen in the γ-amino group in the side chain of the adjacent amino acid residue. For the amino acid residue whose nitrogen in the γ-amino group in the side chain participates in forming the ring, the nitrogen in the α-amino group of this amino acid residue does not participate directly in forming the ring and serves as the linking nitrogen (thus, referred to as the “linking nitrogen” herein) that links one cyclic heptapeptide or polymyxin core to an Fc domain by way of a linker and/or one or two peptides (e.g., a peptide including a 1-5 amino acid residue(s)), or in the case of a conjugate including an Fc domain covalently linked to one or more dimers of cyclic heptapeptides or polymyxin cores, the linking nitrogen serves to link one cyclic heptapeptide or polymyxin core to another cyclic heptapeptide or polymyxin core. Conjugates described herein are separated into two types: (1) one or more dimers of cyclic heptapeptides conjugated to an Fc domain and (2) one or more monomers of cyclic heptapeptides conjugated to an Fc domain. The dimers of cyclic heptapeptides (e.g., two polymyxin cores) are linked to each other at their linking nitrogens through a linker and/or one or two peptides (e.g., a peptide including a 1-5 amino acid residue(s)).

In some embodiments, a peptide including one or more (e.g., 1-5; 1, 2, 3, 4, or 5) amino acid residues (e.g., natural and/or non-natural amino acid residues) may be covalently attached to the linking nitrogen of the cyclic heptapeptide or the polymyxin core.

Cyclic heptapeptides or polymyxin cores may be derived from polymyxins (e.g., naturally existing polymyxins and non-natural polymyxins) and/or octapeptins (e.g., naturally existing octapeptins and non-natural octapeptins). In some embodiments, cyclic heptapeptides may be compounds described in Gallardo-Godoy et al., J. Med. Chem. 59:1068, 2016 (e.g., compounds 11-41 in Table 1 of Gallardo-Godoy et al.), which is incorporated herein by reference in its entirety. Examples of naturally existing polymyxins and their structures are shown in Table 1A. Examples of non-natural polymyxins and their structures are shown in Table 1B.

TABLE 1A
Natural polymyxins and their structures

| | 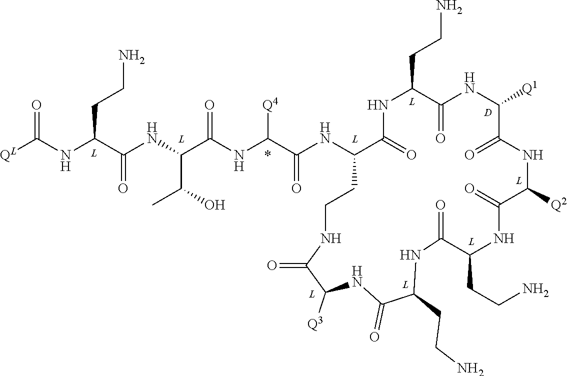 | | | | |
|---|---|---|---|---|---|
| Com- | | | | | |
| pound | Q^{L} | Q⁴ | Q¹ | Q² | Q³ |
| polymyxin B₁ | 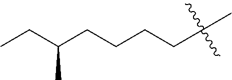 [SMILES: *CCCC[C@@H](C)CC] | 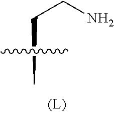 | 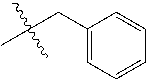 [SMILES: *Cc1ccccc1] | 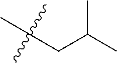 [SMILES: *CC(C)C] | 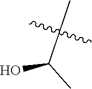 [SMILES: *[C@@H](C)O] |
| polymyxin B₂ | 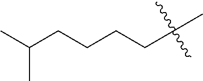 [SMILES: *CCCCC(C)C] | 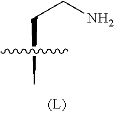 | 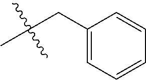 [SMILES: *Cc1ccccc1] | 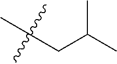 [SMILES: *CC(C)C] | 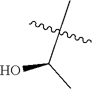 [SMILES: *[C@@H](C)O] |
| polymyxin B₃ | 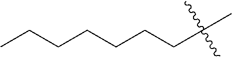 [SMILES: *CCCCCCC] | 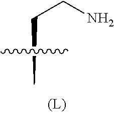 | 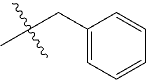 [SMILES: *Cc1ccccc1] | 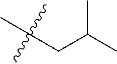 [SMILES: *CC(C)C] | 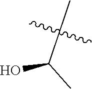 [SMILES: *[C@@H](C)O] |
| polymyxin B₄ | 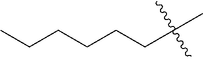 [SMILES: *CCCCCC] | 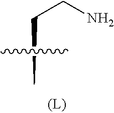 | 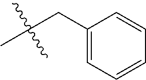 [SMILES: *Cc1ccccc1] | 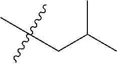 [SMILES: *CC(C)C] | 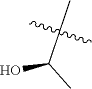 [SMILES: *[C@@H](C)O] |
| polymyxin B₅ | 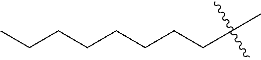 [SMILES: *CCCCCCCC] | 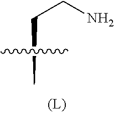 | 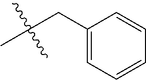 [SMILES: *Cc1ccccc1] | 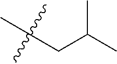 [SMILES: *CC(C)C] | 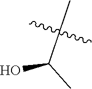 [SMILES: *[C@@H](C)O] |
| polymyxin B₆ | 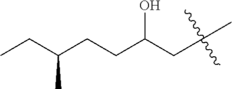 [SMILES: *CC(O)CC[C@@H](C)CC] | 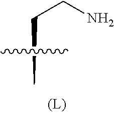 | 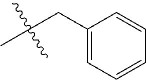 [SMILES: *Cc1ccccc1] | 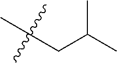 [SMILES: *CC(C)C] | 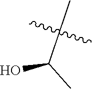 [SMILES: *[C@@H](C)O] |
| polymyxin B₁-Ile | 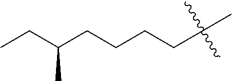 [SMILES: *CCCC[C@@H](C)CC] | 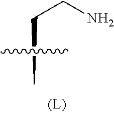 | 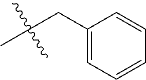 [SMILES: *Cc1ccccc1] | 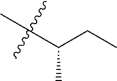 [SMILES: *[C@@H](C)CC] | 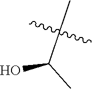 [SMILES: *[C@@H](C)O] |
| polymyxin B₂-Ile | 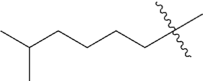 [SMILES: *CCCCC(C)C] | 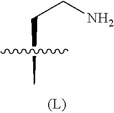 | 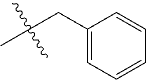 [SMILES: *Cc1ccccc1] | 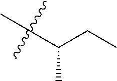 [SMILES: *[C@@H](C)CC] | 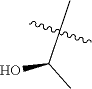 [SMILES: *[C@@H](C)O] |
| polymyxin C₁ | 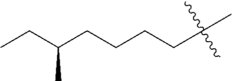 [SMILES: *CCCC[C@@H](C)CC] | 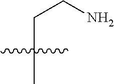 [SMILES: *CCN] | 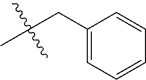 [SMILES: *Cc1ccccc1] | 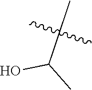 [SMILES: *C(C)O] | 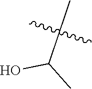 [SMILES: *C(C)O] |
| polymyxin C₂ | 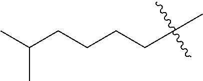 [SMILES: *CCCCC(C)C] | 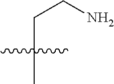 [SMILES: *CCN] | 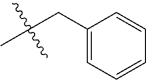 [SMILES: *Cc1ccccc1] | 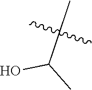 [SMILES: *C(C)O] | 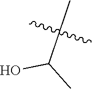 [SMILES: *C(C)O] |
| polymyxin S₁ | 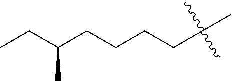 [SMILES: *CCCC[C@@H](C)CC] | 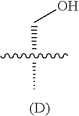 | 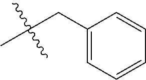 [SMILES: *Cc1ccccc1] | 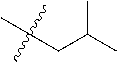 [SMILES: *CC(C)C] | 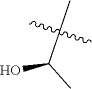 [SMILES: *[C@@H](C)O] |
| polymyxin T₁ | 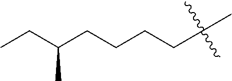 [SMILES: *CCCC[C@@H](C)CC] | 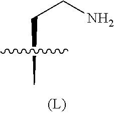 | 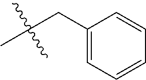 [SMILES: *Cc1ccccc1] | 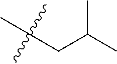 [SMILES: *CC(C)C] | 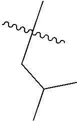 [SMILES: *CC(C)C] |
| polymyxin T₂ | 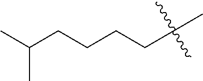 [SMILES: *CCCCC(C)C] | 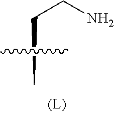 | 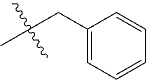 [SMILES: *Cc1ccccc1] | 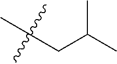 [SMILES: *CC(C)C] | 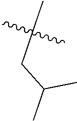 [SMILES: *CC(C)C] |
| polymyxin A₁ | 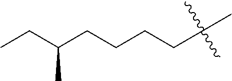 [SMILES: *CCCC[C@@H](C)CC] | 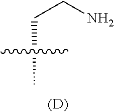 | 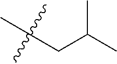 [SMILES: *CC(C)C] | 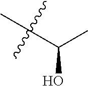 [SMILES: *[C@@H](C)O] | 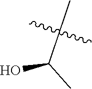 [SMILES: *[C@@H](C)O] |
| polymyxin A₂ | 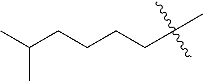 [SMILES: *CCCCC(C)C] | 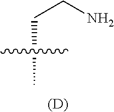 | 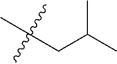 [SMILES: *CC(C)C] | 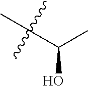 [SMILES: *[C@@H](C)O] | 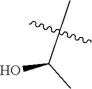 [SMILES: *[C@@H](C)O] |
| polymyxin D₁ | 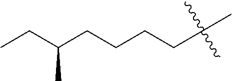 [SMILES: *CCCC[C@@H](C)CC] | 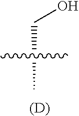 | 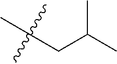 [SMILES: *CC(C)C] | 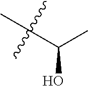 [SMILES: *[C@@H](C)O] | 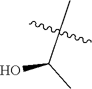 [SMILES: *[C@@H](C)O] |
| polymyxin D₂ | 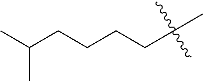 [SMILES: *CCCCC(C)C] | 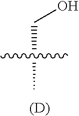 | 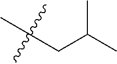 [SMILES: *CC(C)C] | 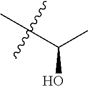 [SMILES: *[C@@H](C)O] | 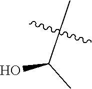 [SMILES: *[C@@H](C)O] |
| polymyxin E₁ (colistin A) | 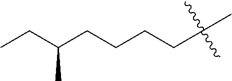 [SMILES: *CCCC[C@@H](C)CC] | 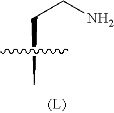 | 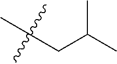 [SMILES: *CC(C)C] | 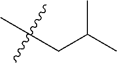 [SMILES: *CC(C)C] | 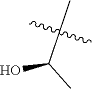 [SMILES: *[C@@H](C)O] |
| polymyxin E₂ (colistin B) | 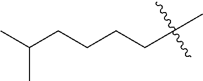 [SMILES: *CCCCC(C)C] | 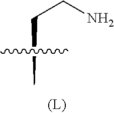 | 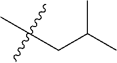 [SMILES: *CC(C)C] | 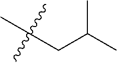 [SMILES: *CC(C)C] | 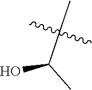 [SMILES: *[C@@H](C)O] |
| polymyxin E₃ | 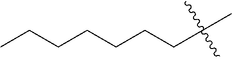 [SMILES: *CCCCCCC] | 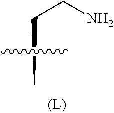 | 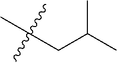 [SMILES: *CC(C)C] | 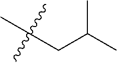 [SMILES: *CC(C)C] | 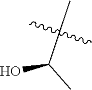 [SMILES: *[C@@H](C)O] |
| polymyxin E₄ | 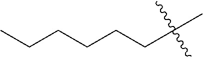 [SMILES: *CCCCCC] | 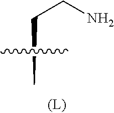 | 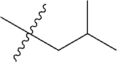 [SMILES: *CC(C)C] | 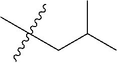 [SMILES: *CC(C)C] | 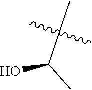 [SMILES: *[C@@H](C)O] |
| polymyxin E₇ | 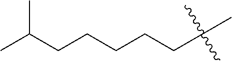 [SMILES: *CCCCCC(C)C] | 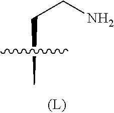 | 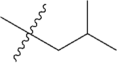 [SMILES: *CC(C)C] | 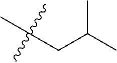 [SMILES: *CC(C)C] | 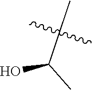 [SMILES: *[C@@H](C)O] |
| polymyxin E₁-Ile | 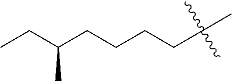 [SMILES: *CCCC[C@@H](C)CC] | 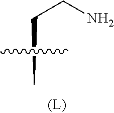 | 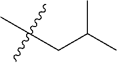 [SMILES: *CC(C)C] | 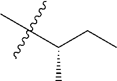 [SMILES: *[C@@H](C)CC] | 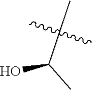 [SMILES: *[C@@H](C)O] |
| polymyxin E₁-Val | 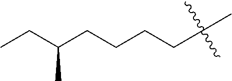 [SMILES: *CCCC[C@@H](C)CC] | 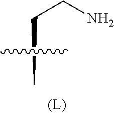 | 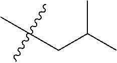 [SMILES: *CC(C)C] | 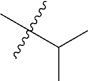 [SMILES: *C(C)C] | 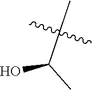 [SMILES: *[C@@H](C)O] |
| polymyxin E₁-Nva | 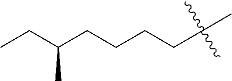 [SMILES: *CCCC[C@@H](C)CC] | 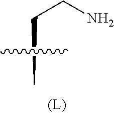 | 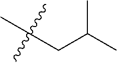 [SMILES: *CC(C)C] | 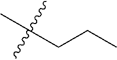 [SMILES: *CCC] | 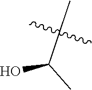 [SMILES: *[C@@H](C)O] |
| polymyxin E₂-Ile | 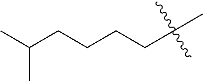 [SMILES: *CCCCC(C)C] | 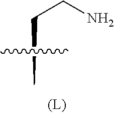 | 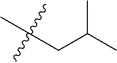 [SMILES: *CC(C)C] | 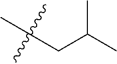 [SMILES: *CC(C)C] | 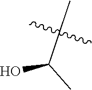 [SMILES: *[C@@H](C)O] |
| polymyxin E₂-Val | 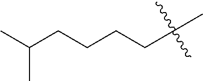 [SMILES: *CCCCC(C)C] | 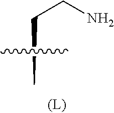 | 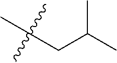 [SMILES: *CC(C)C] | 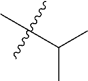 [SMILES: *C(C)C] | 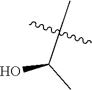 [SMILES: *[C@@H](C)O] |
| polymyxin E₂-Nva | 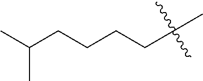 [SMILES: *CCCCC(C)C] | 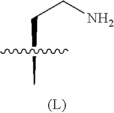 | 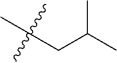 [SMILES: *CC(C)C] | 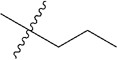 [SMILES: *CCC] | 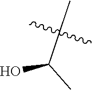 [SMILES: *[C@@H](C)O] |
| polymyxin E₈-Ile | 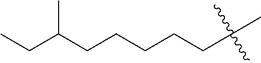 [SMILES: *CCCCCC(C)CC] | 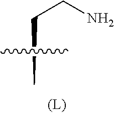 | 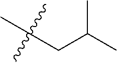 [SMILES: *CC(C)C] | 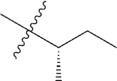 [SMILES: *[C@@H](C)CC] | 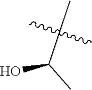 [SMILES: *[C@@H](C)O] |
| polymyxin M₁ | 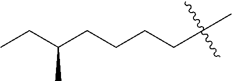 [SMILES: *CCCC[C@@H](C)CC] | 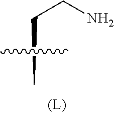 | 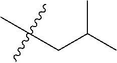 [SMILES: *CC(C)C] | 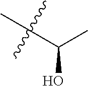 [SMILES: *[C@@H](C)O] | 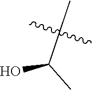 [SMILES: *[C@@H](C)O] |
| polymyxin M₂ | 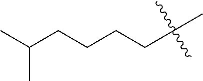 [SMILES: *CCCCC(C)C] | 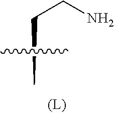 | 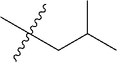 [SMILES: *CC(C)C] | 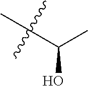 [SMILES: *[C@@H](C)O] | 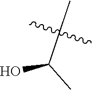 [SMILES: *[C@@H](C)O] |

* = stereochemistry varies and is indicated herein when known

TABLE 1B
Non-natural polymyxins and their structures

| | 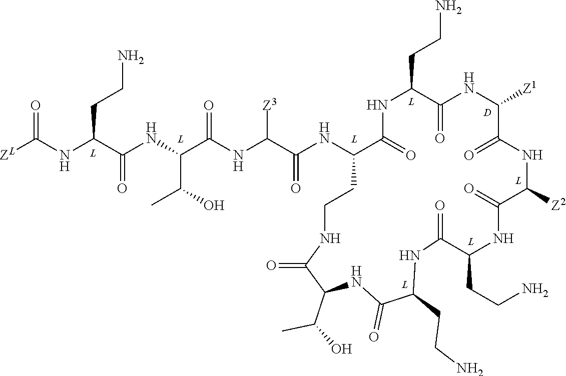 | | | |
|---|---|---|---|---|
| Non-natural | | | | |
| Compound | Z^{L} | Z³ | Z¹ | Z² |
| (i) | Octanoyl | L-Dab | D-Leu | L-Thr |
| (ii) | Octanoyl | L-Dab | D-Leu | L-Ala |
| (iii) | Octanoyl | L-Dab | D-Leu | L-Val |
| (iv) | Octanoyl | L-Dab | D-Nle | L-Thr |
| (v) | Octanoyl | L-Dab | D-Leu | L-Ser |
| (vi) | Octanoyl | L-Dab | D-Thr | L-Leu |
| (vii) | Octanoyl | L-Dab | D-Val | L-Thr |
| (viii) | Octanoyl | L-Dab | D-Thr | L-Thr |
| (ix) | Octanoyl | L-Dab | D-Val | L-Val |
| (x) | Octanoyl | L-Dap | D-Leu | L-Thr |
| (xi) | Octanoyl | L-Orn | D-Leu | L-Thr |
| (xii) | Octanoyl | L-Dap | D-Leu | L-Ala |
| (xiii) | Octanoyl | L-Dap | D-Leu | L-Abu |
| (xiv) | Octanoyl | D-Dap | D-Leu | L-Abu |
| (xv) | Octanoyl | L-Dab | D-Leu | L-Abu |
| (xvi) | Octanoyl | L-Dab | Cyclohexylmethyl | L-Thr |
| (xvii) | Octanoyl | L-Dab | Cyclopentylmethyl | L-Thr |
| (xviii) | Octanoyl | L-Dab | Cyclopropylmethyl | L-Thr |
| (xvix) | Octanoyl | L-Dab | Naphthylmethyl | L-Thr |
| (xx) | Octanoyl | L-Dab | Thiophenylmethyl | L-Thr |
| (xi) | Octanoyl | L-Dab | Furanylmethyl | L-Thr |

Dab: diaminobutyric acid;
Dap: diaminopropionic acid;
Orn: ornithine;
Abu: 2-aminobutyric acid;
Nle: norleucine

Conjugates of Dimers of Cyclic Heptapeptides Linked to an Fc Domain

The conjugates described herein an Fc domain covalently linked to one or more dimers of cyclic heptapeptides. The dimers of two cyclic heptapeptides include a first cyclic heptapeptide (e.g., a first polymyxin core) and a second cyclic heptapeptide (e.g., a second polymyxin core). The first and second cyclic heptapeptides are linked to each other by way of a linker and/or one or two peptides (e.g., each peptide including a 1-5 amino acid residue(s)). In some embodiments of the dimers of cyclic heptapeptides, the first and second cyclic heptapeptides are the same. In some embodiments, the first and second cyclic heptapeptides are different. The disclosure provides a conjugate, or a pharmaceutically acceptable salt thereof, described by formula (I):

In some embodiments, the disclosure provides a conjugate, or a pharmaceutically acceptable salt thereof, described by the formulae below:

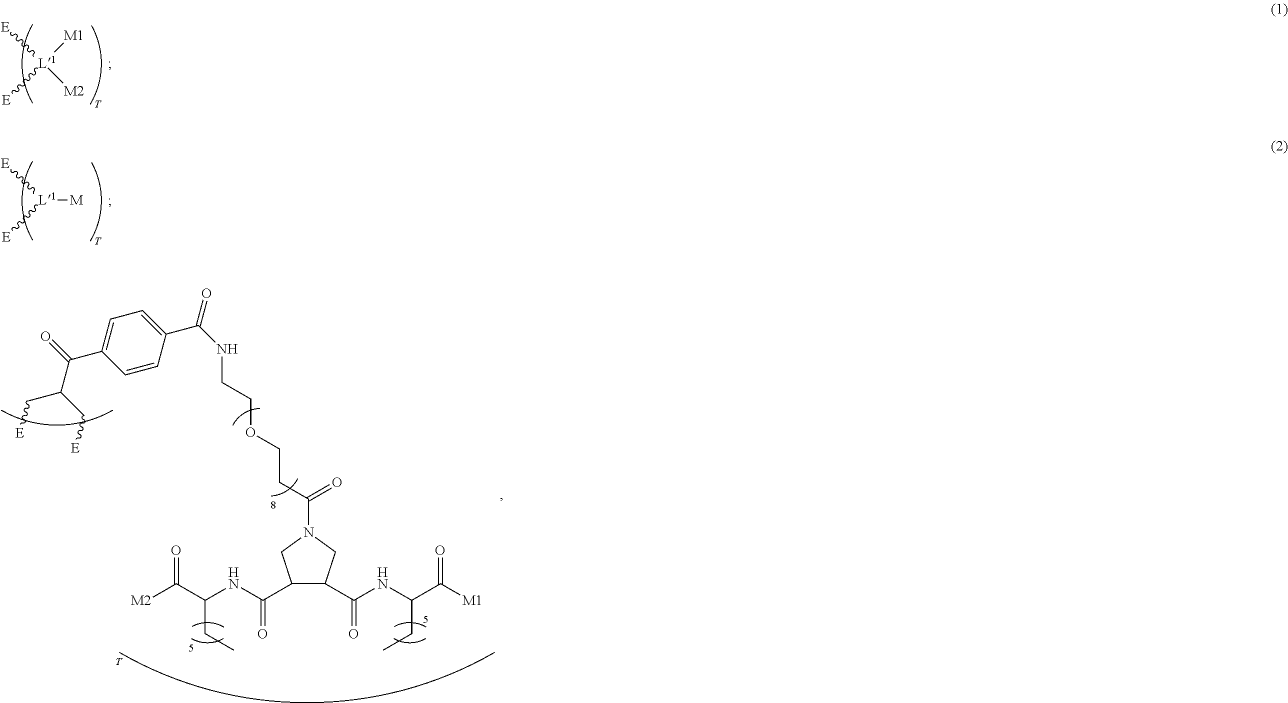

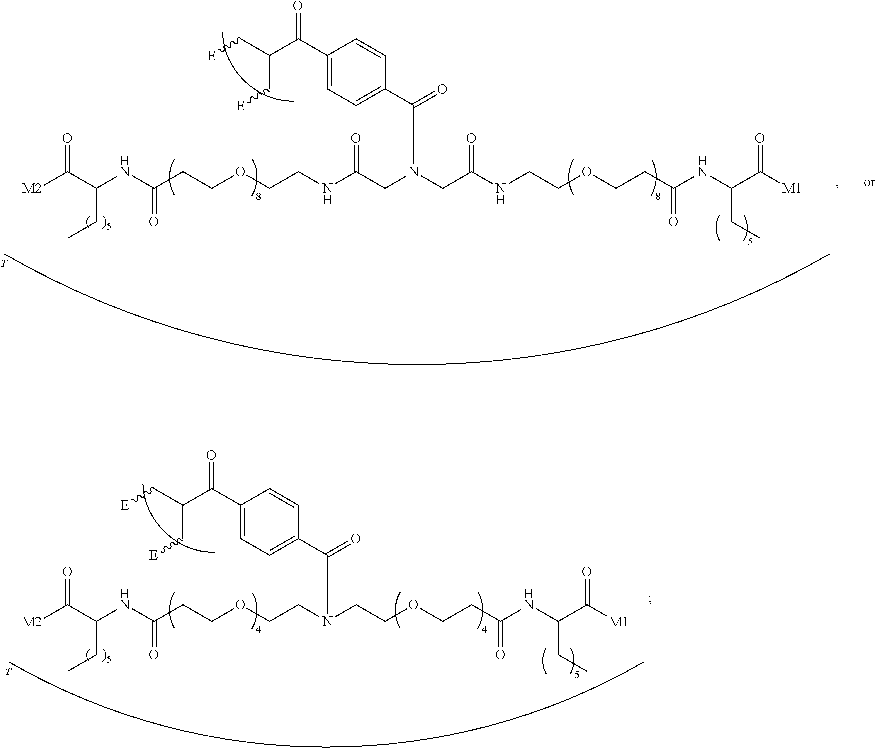

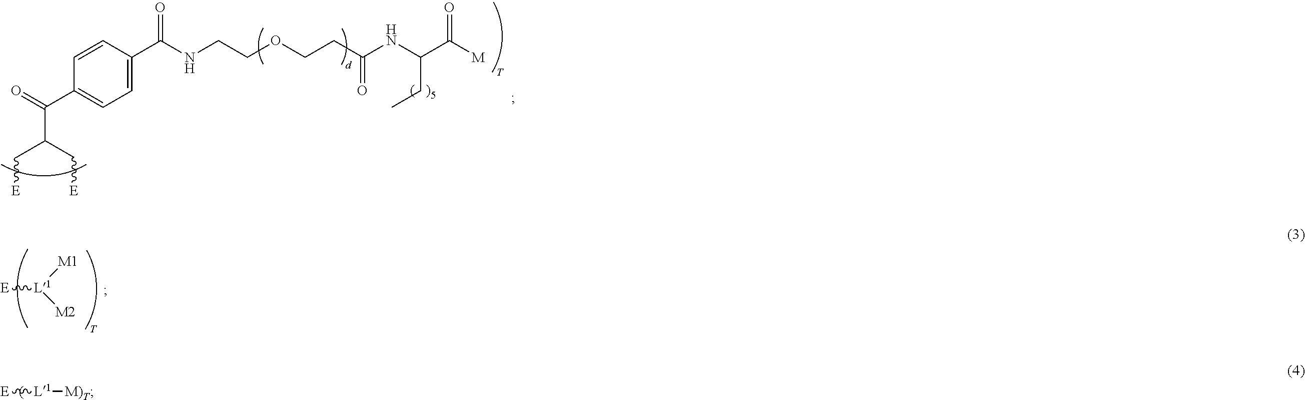

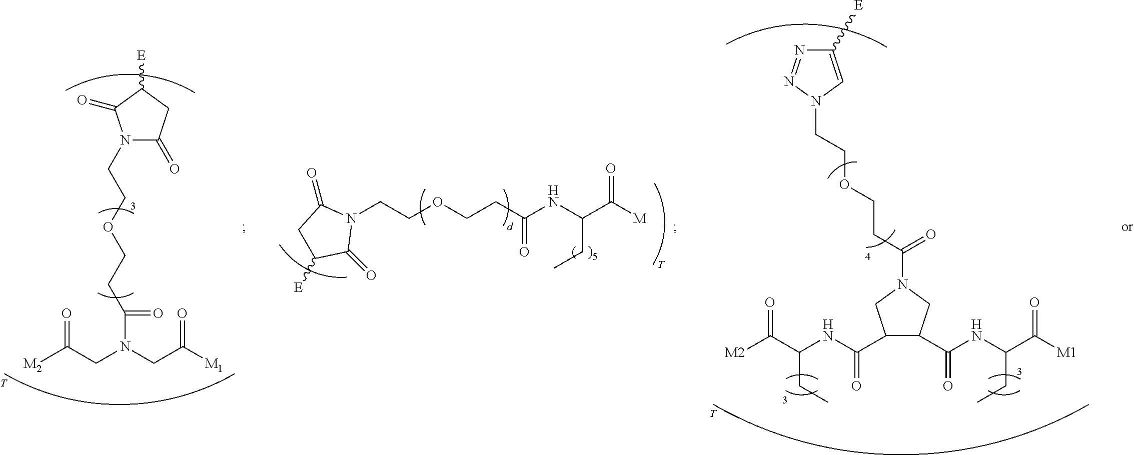

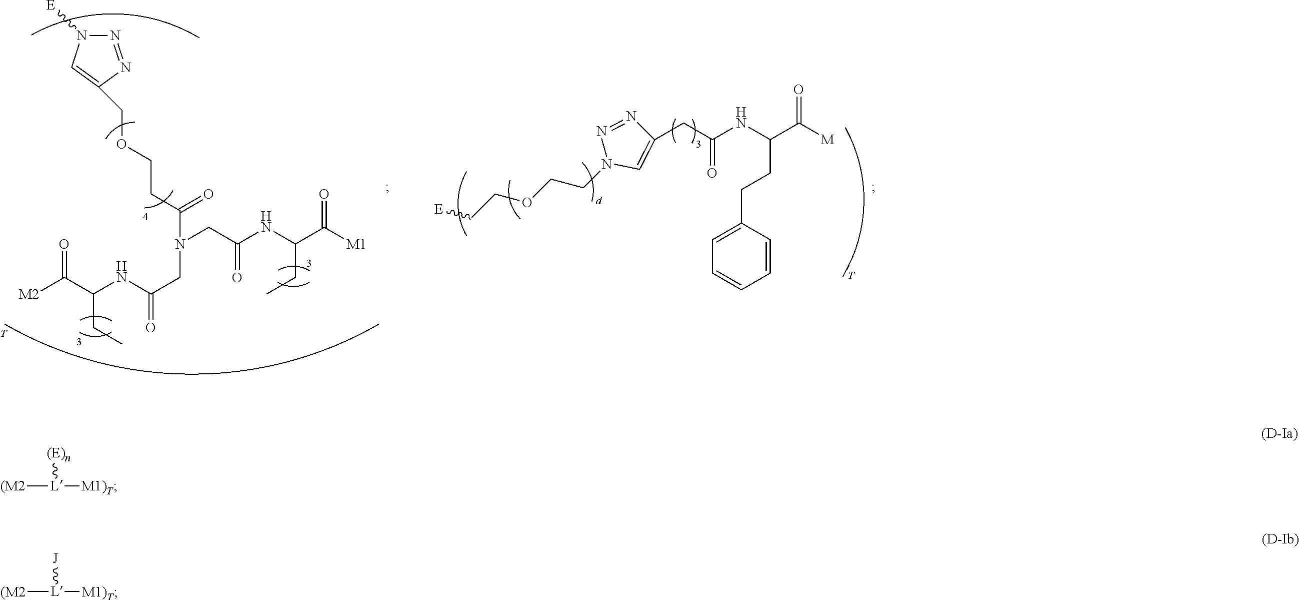

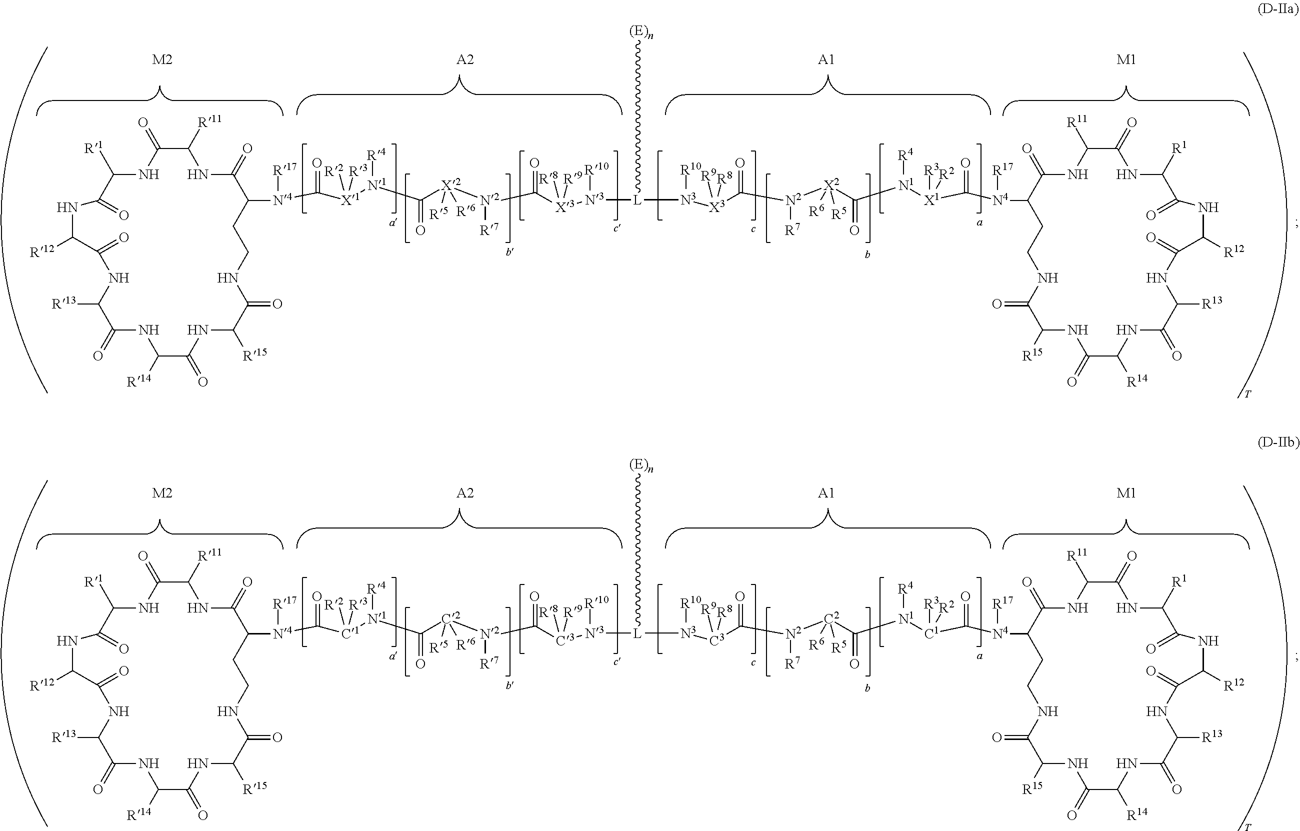

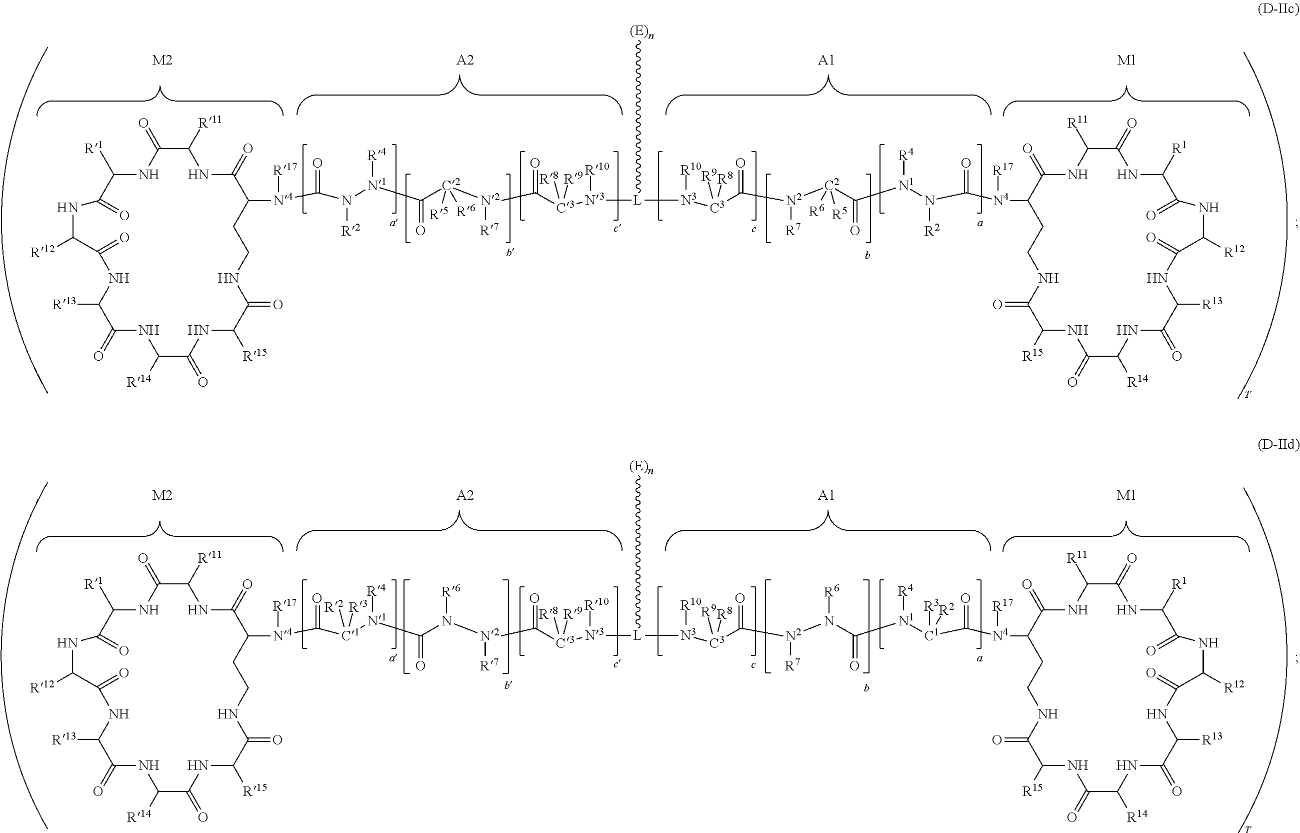

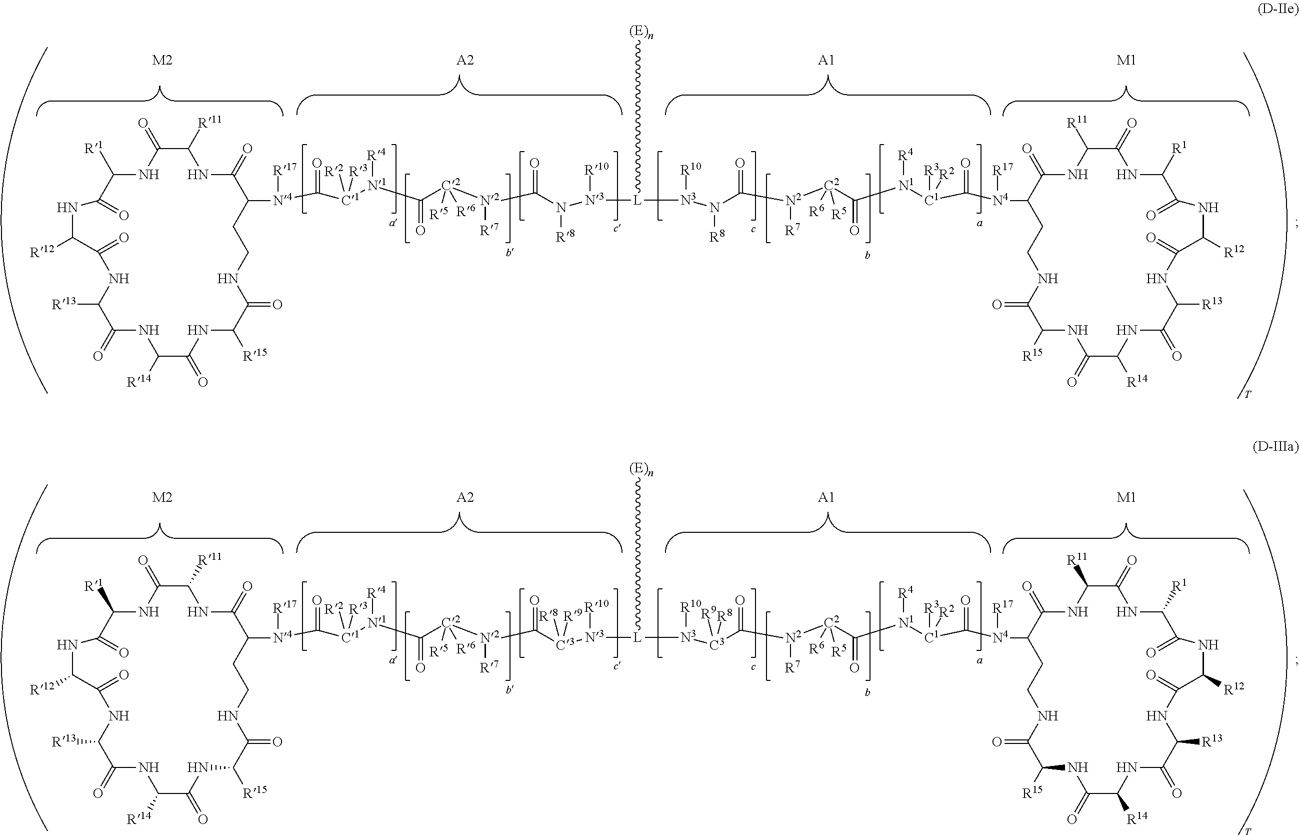

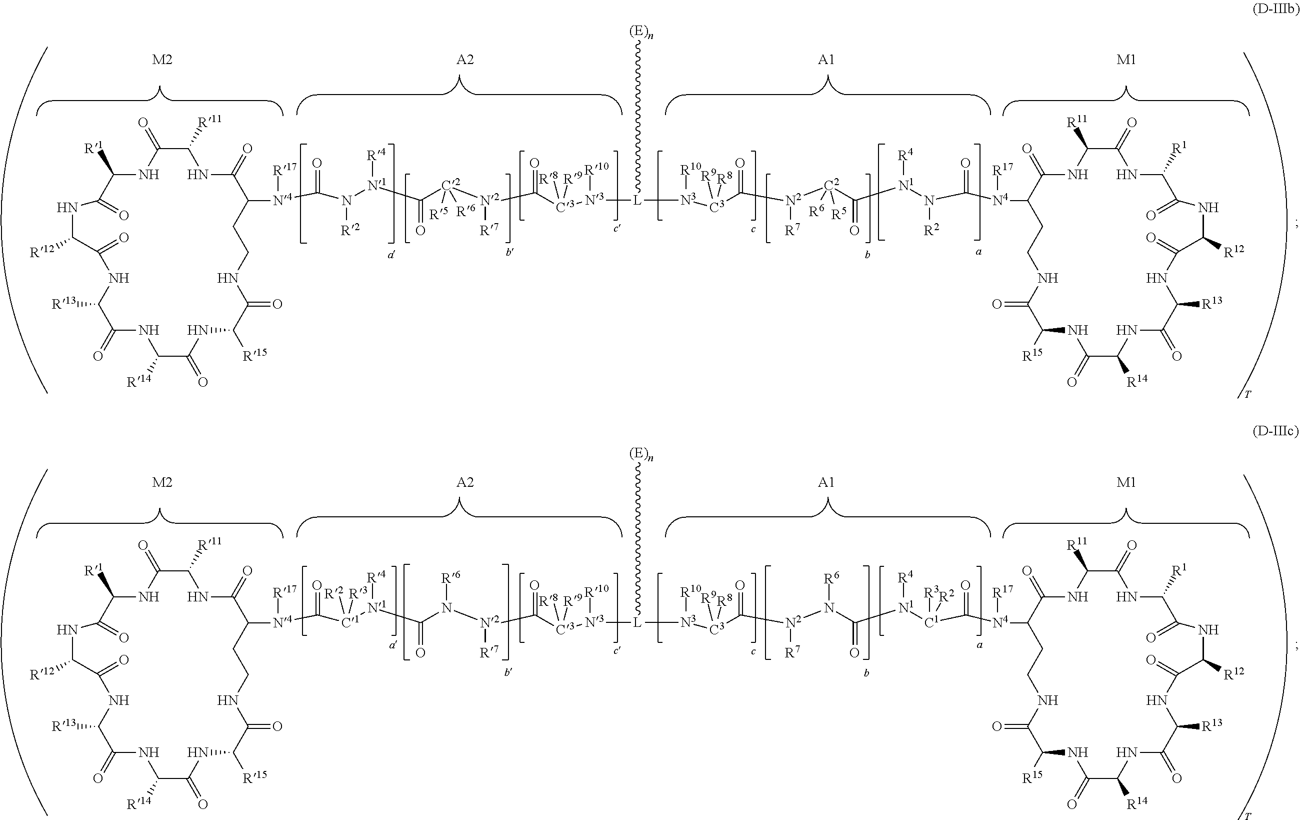

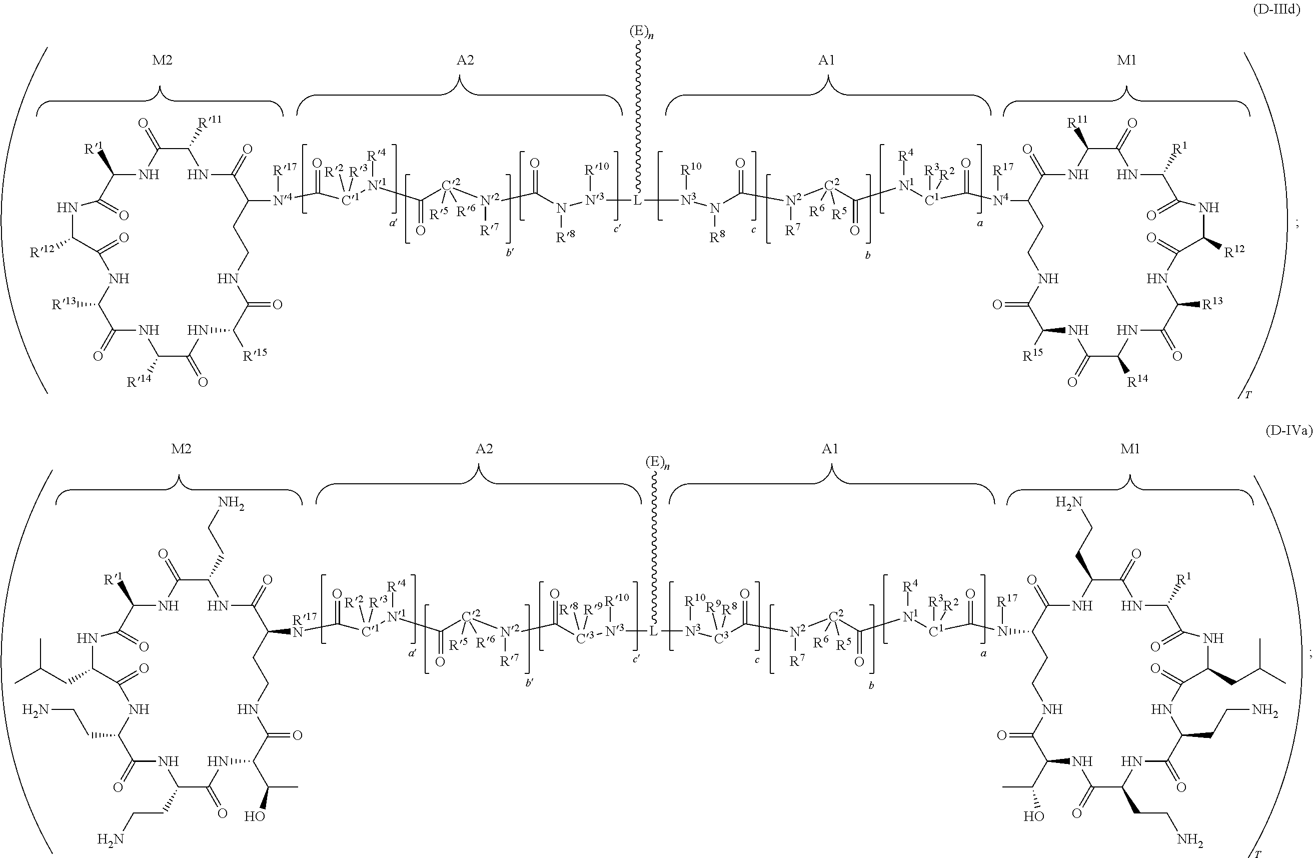

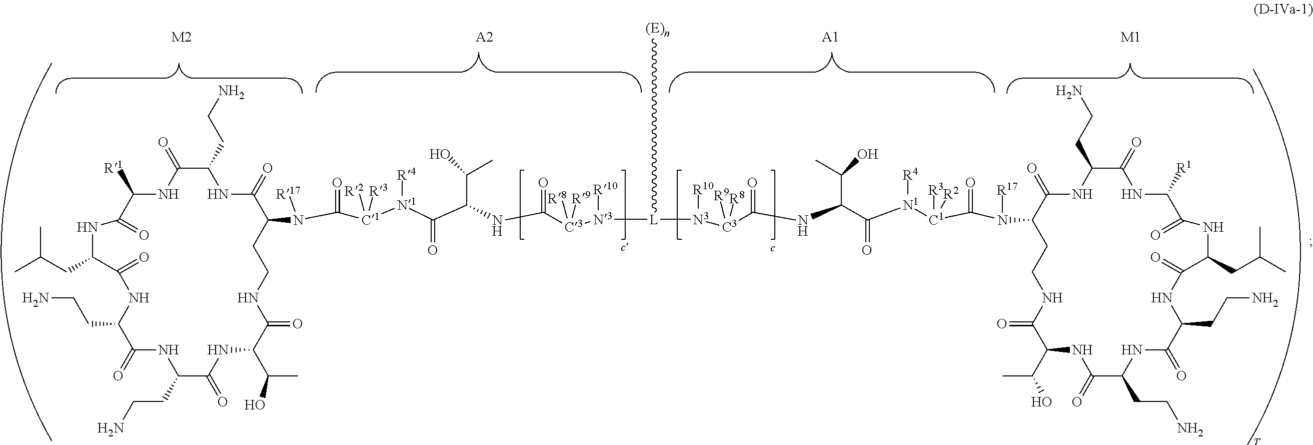

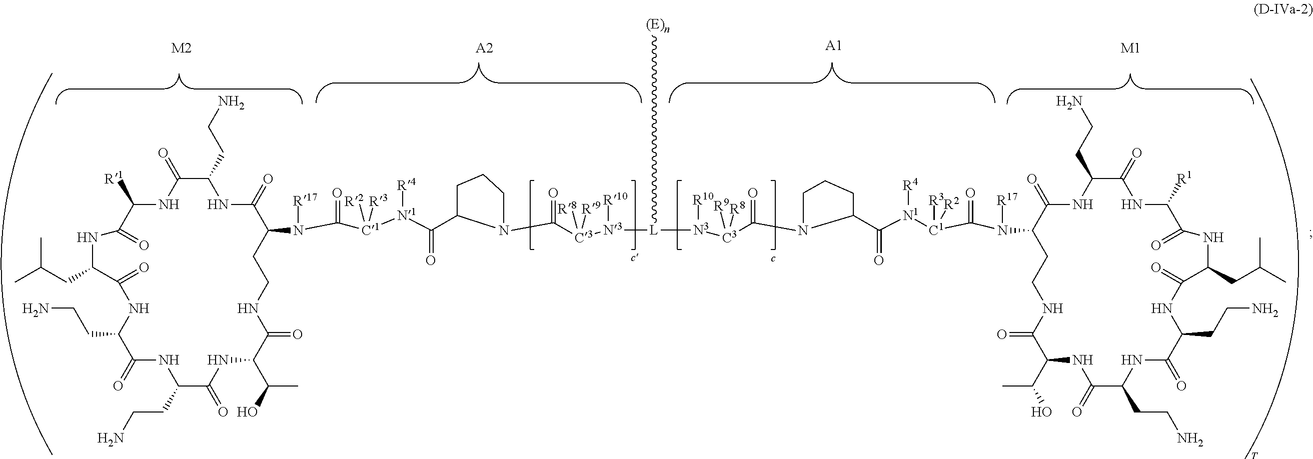

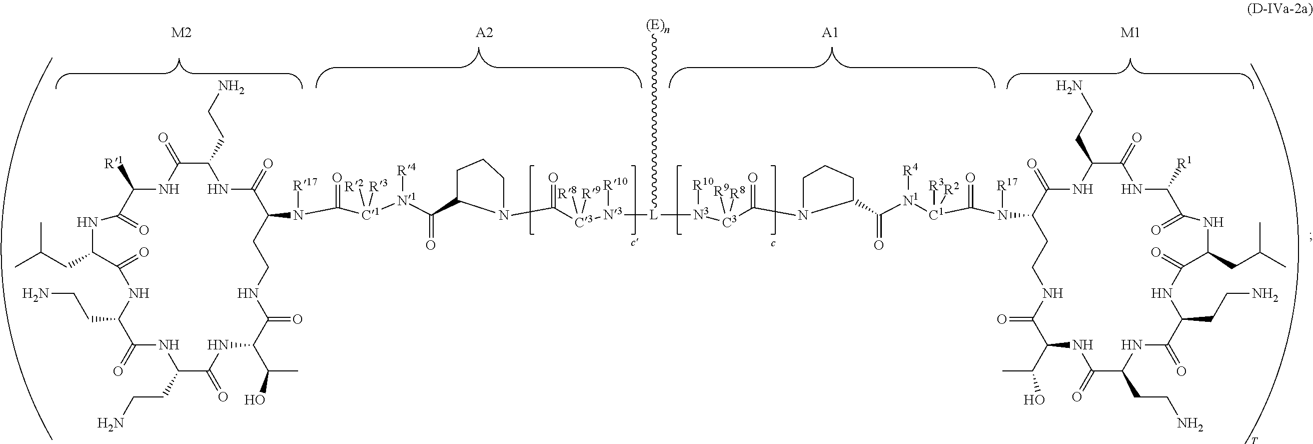

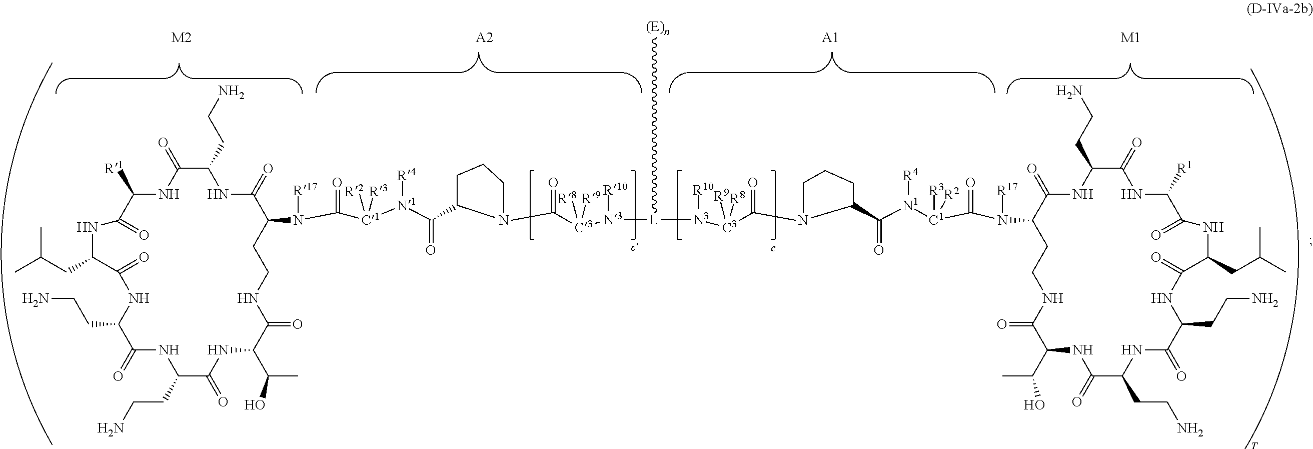

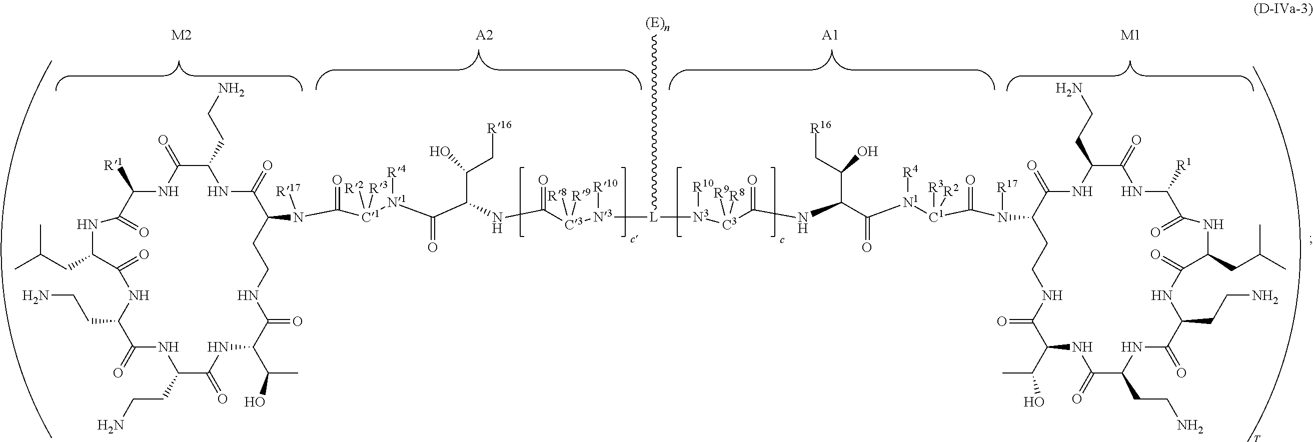

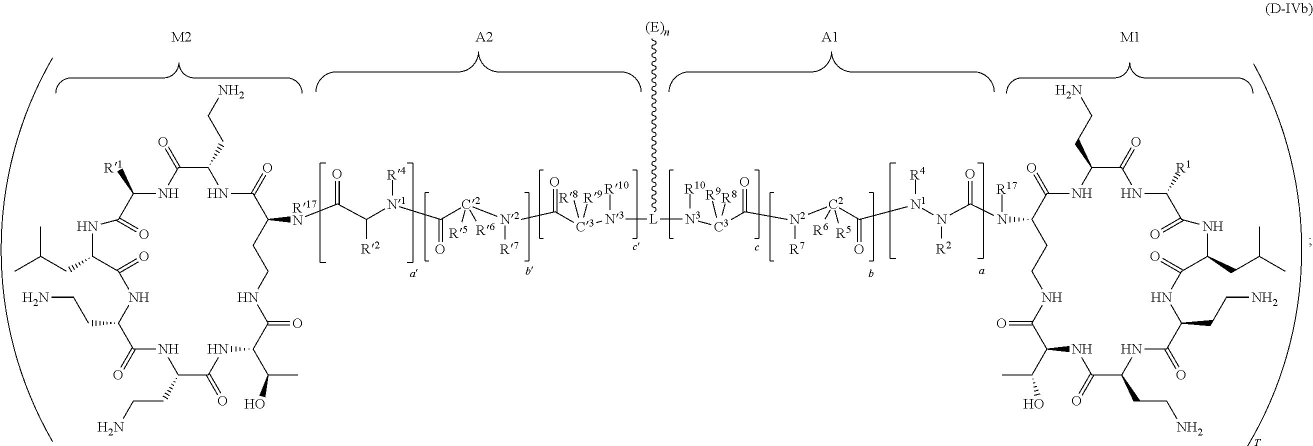

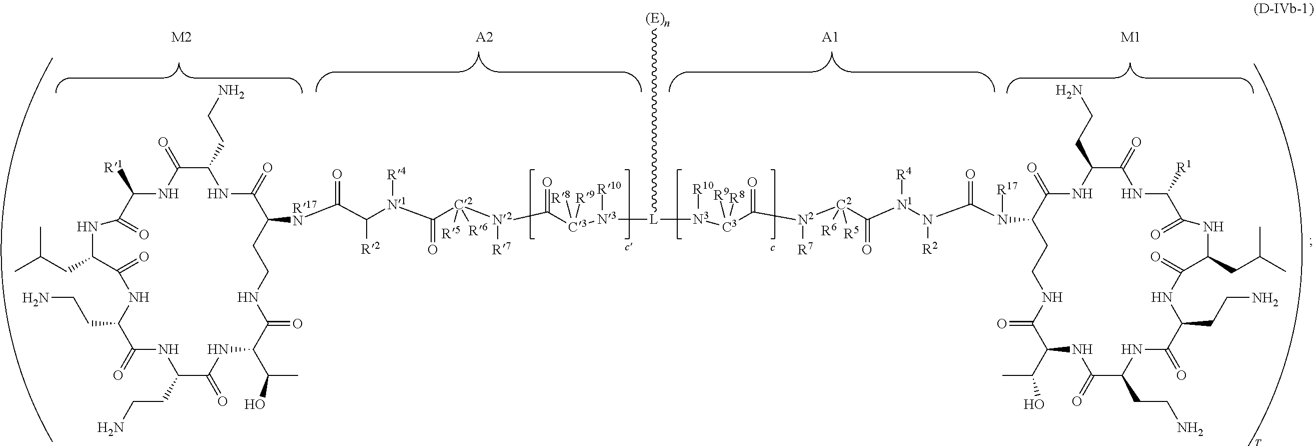

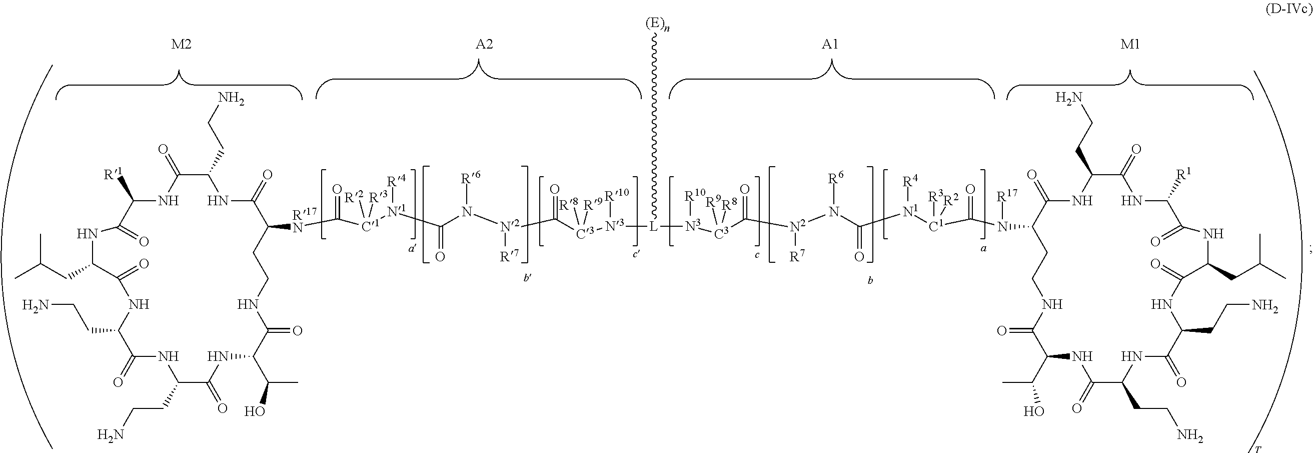

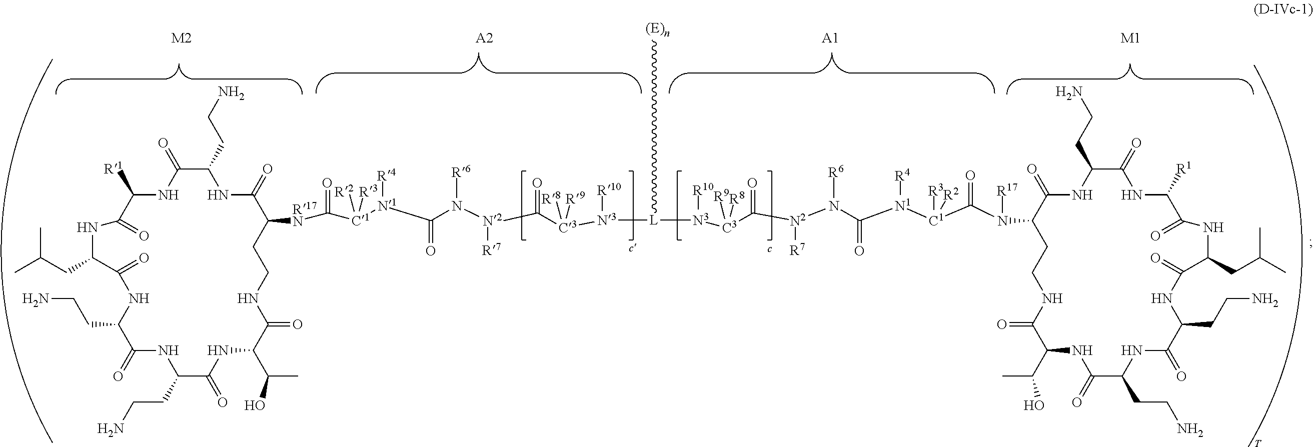

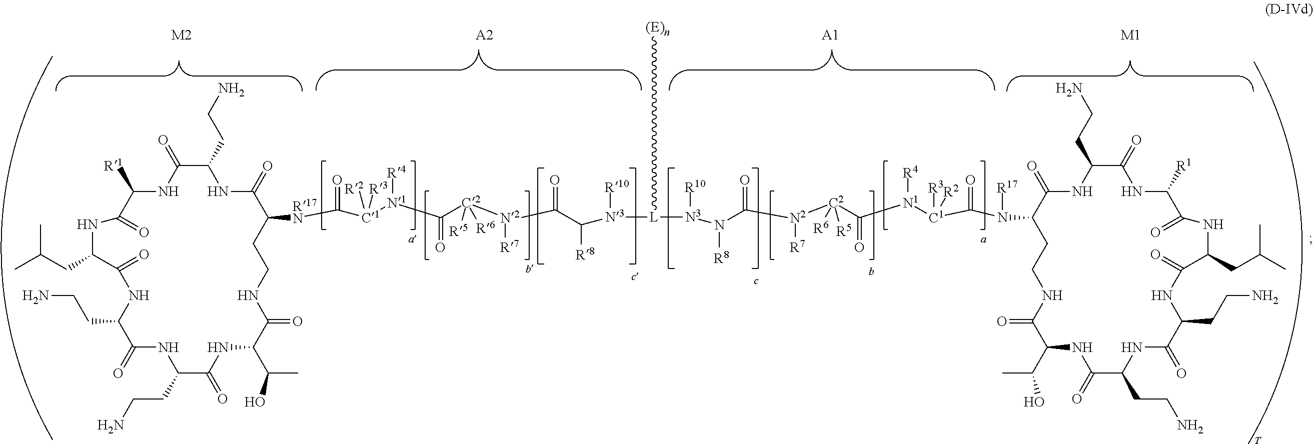

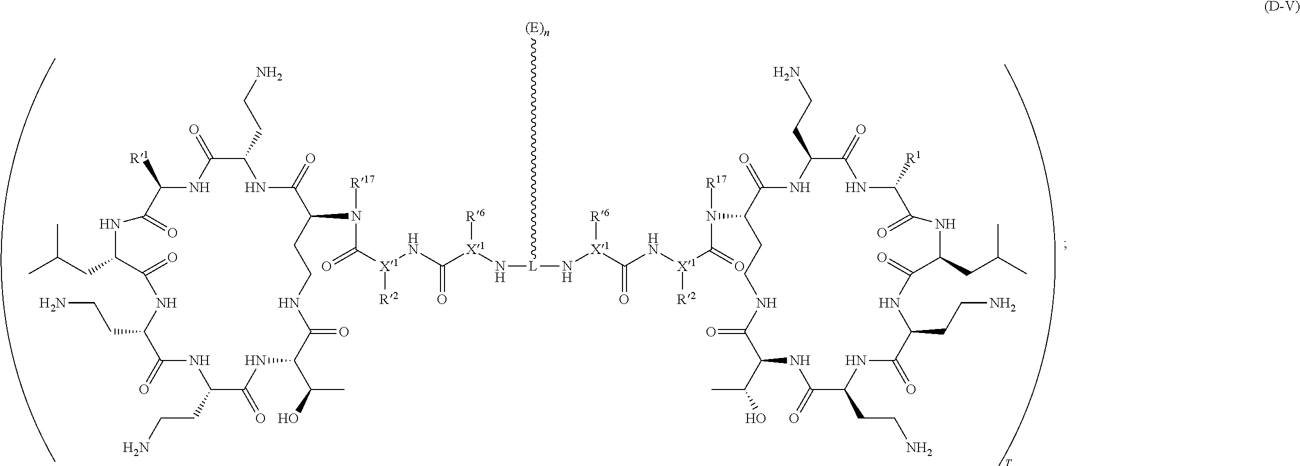

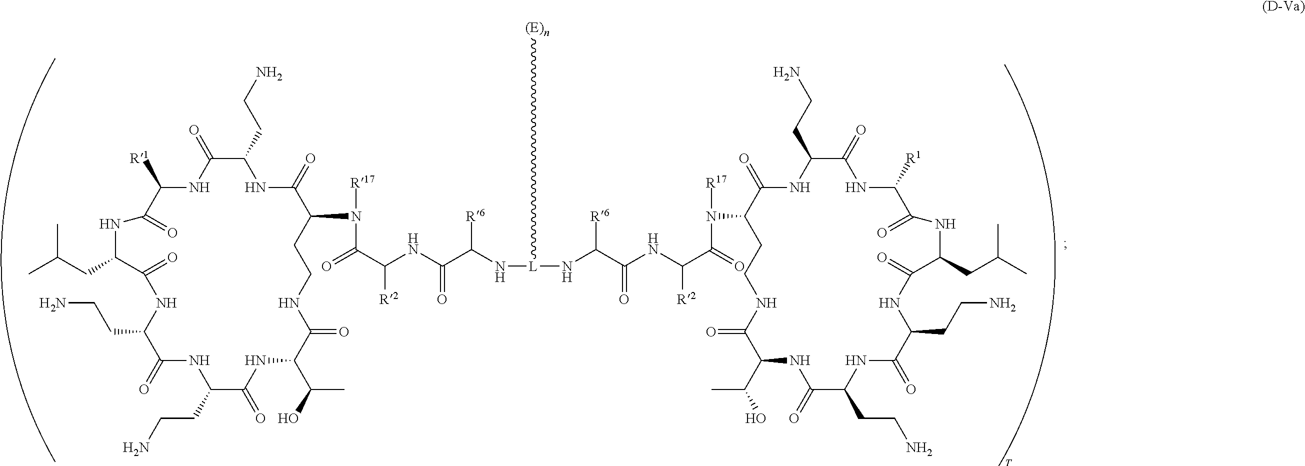

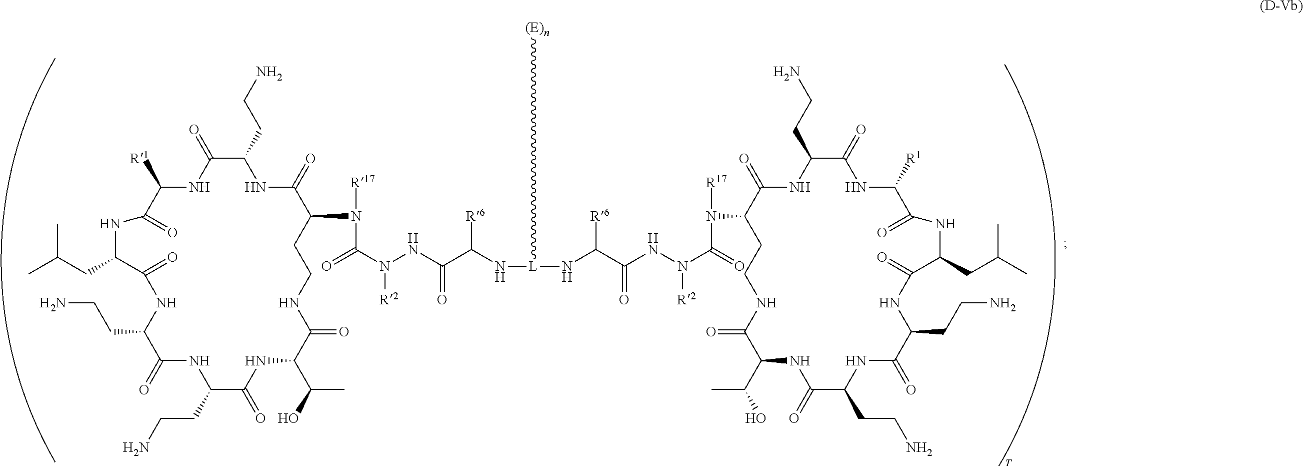

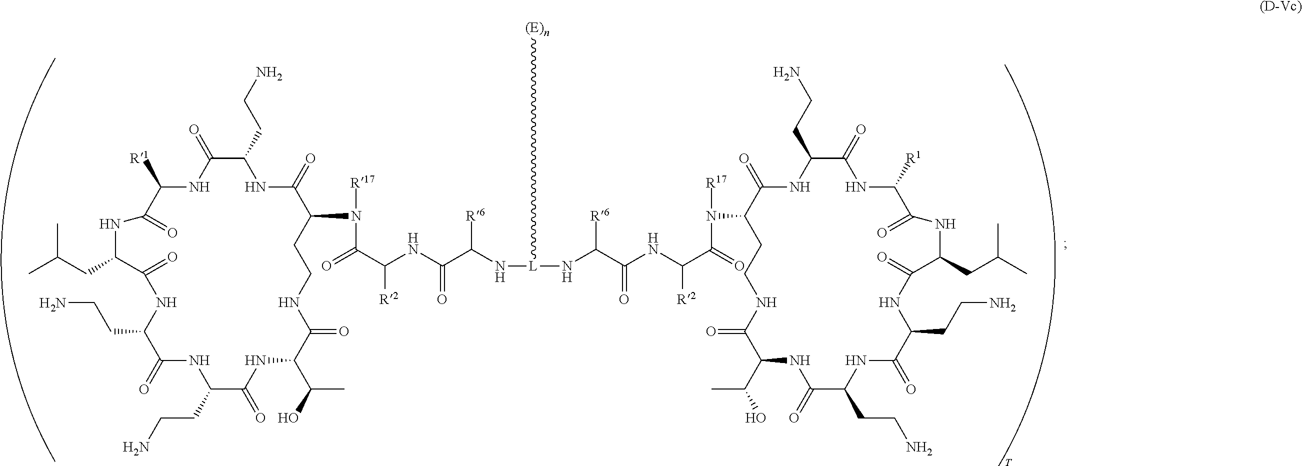

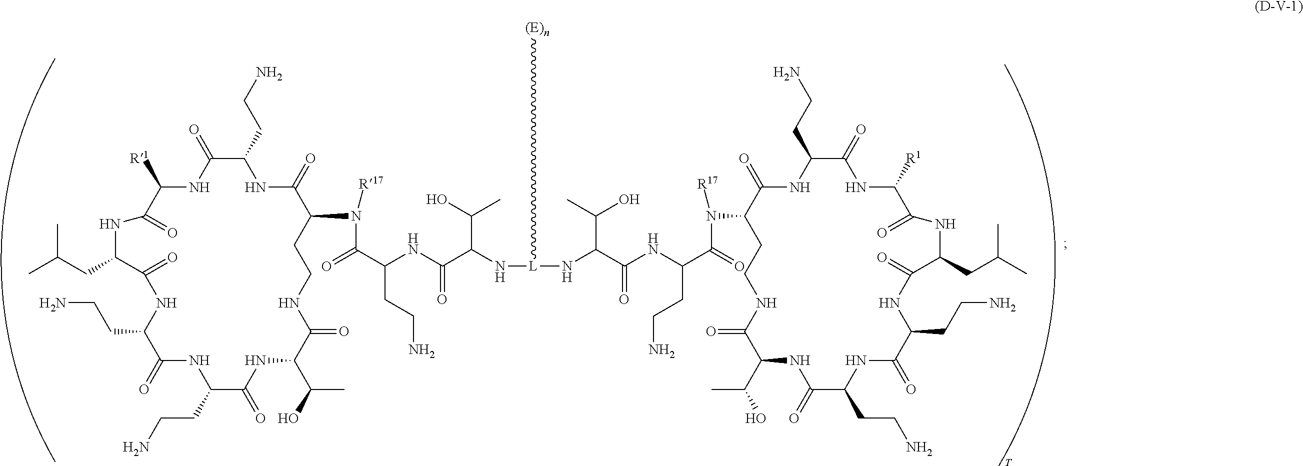

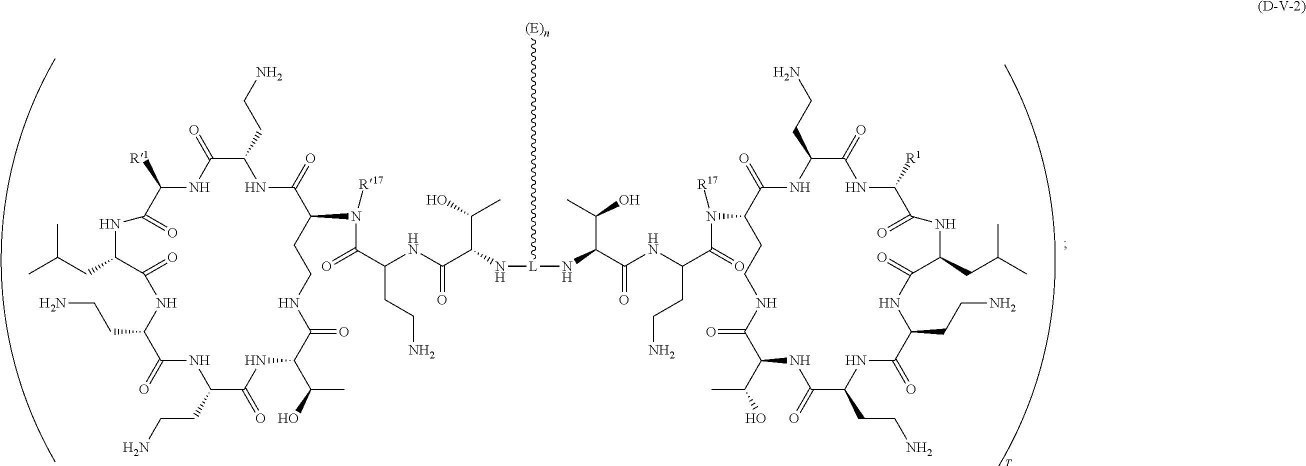

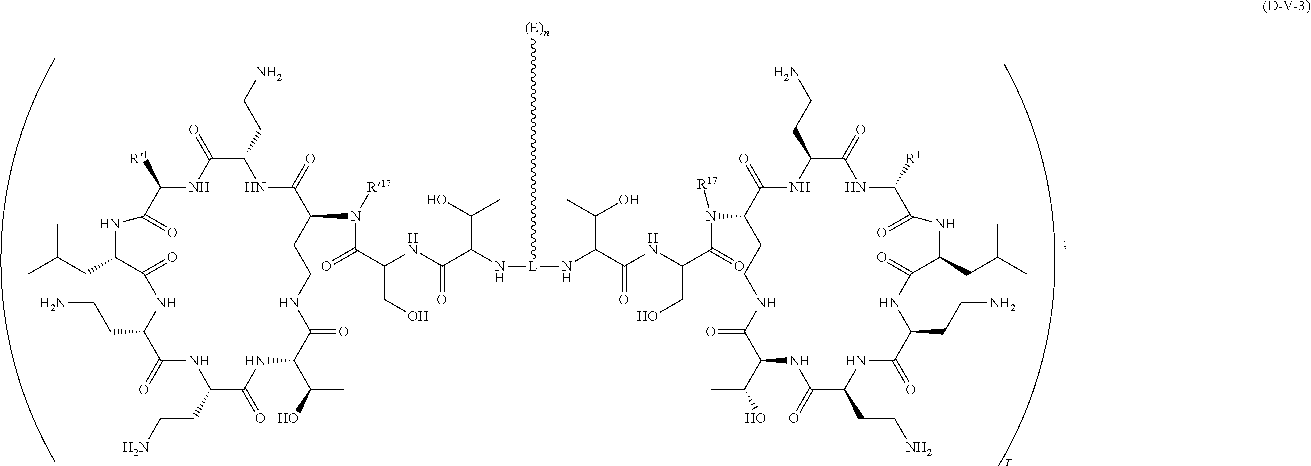

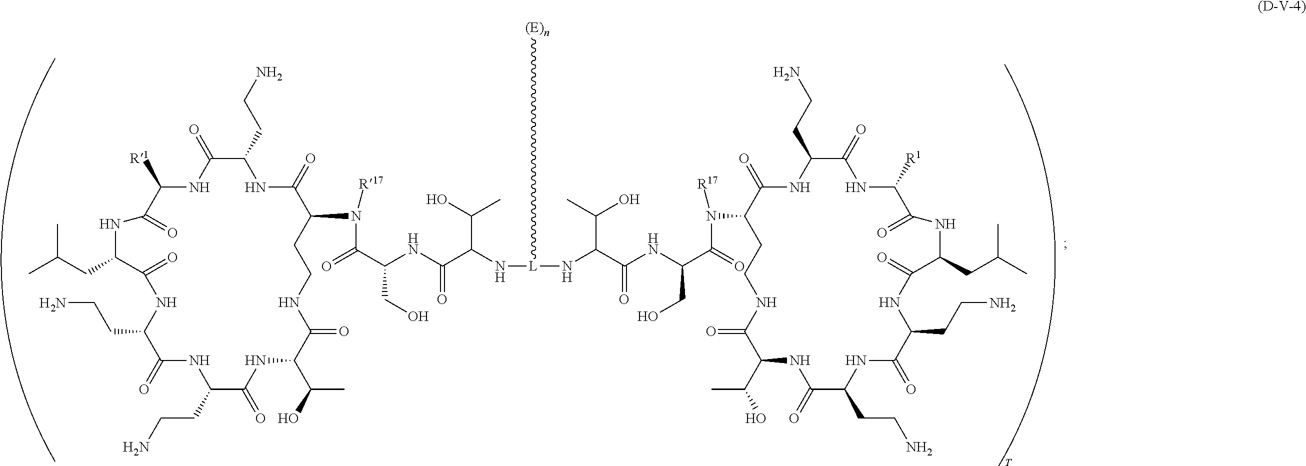

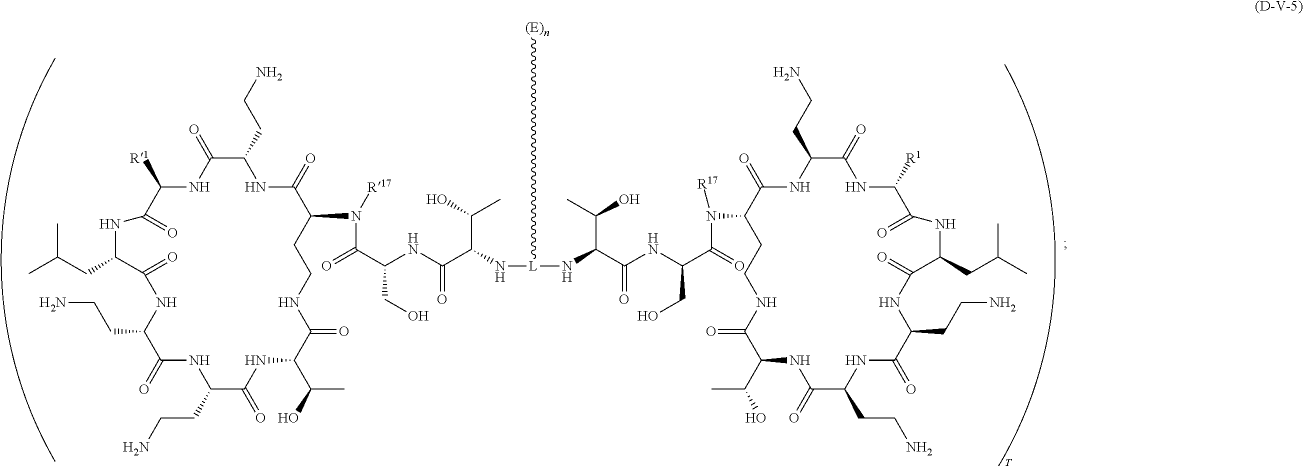

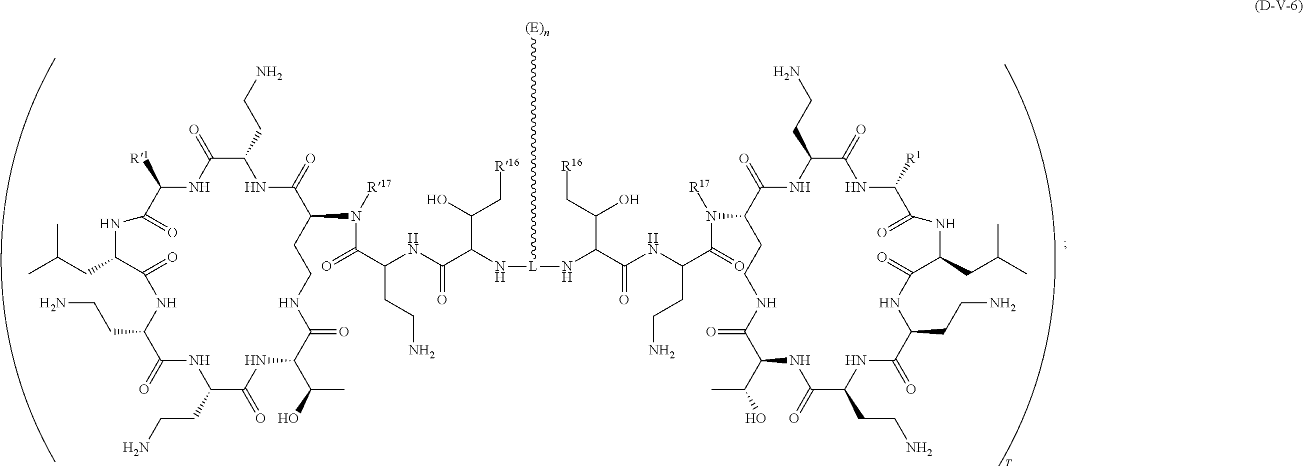

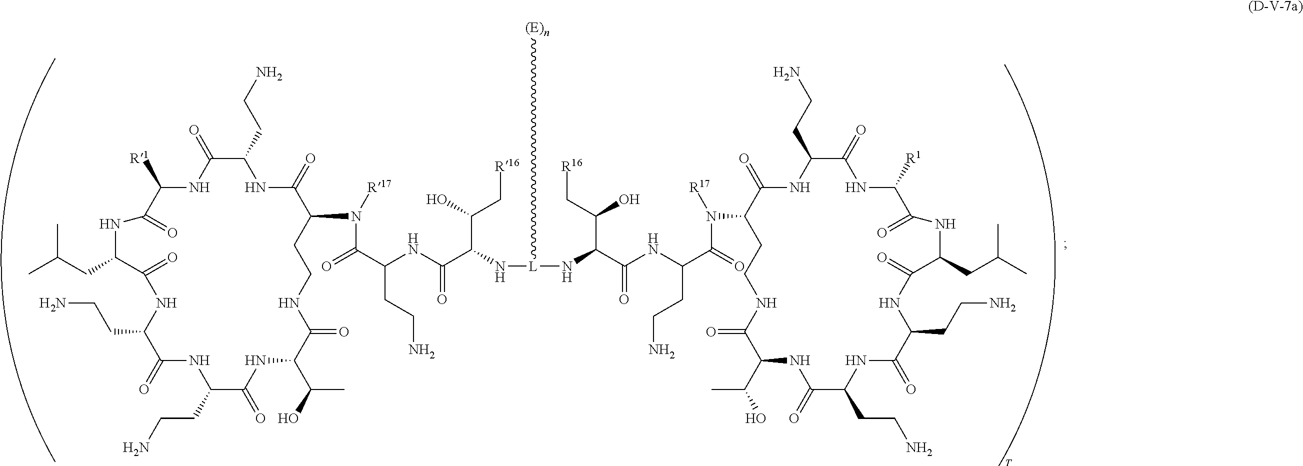

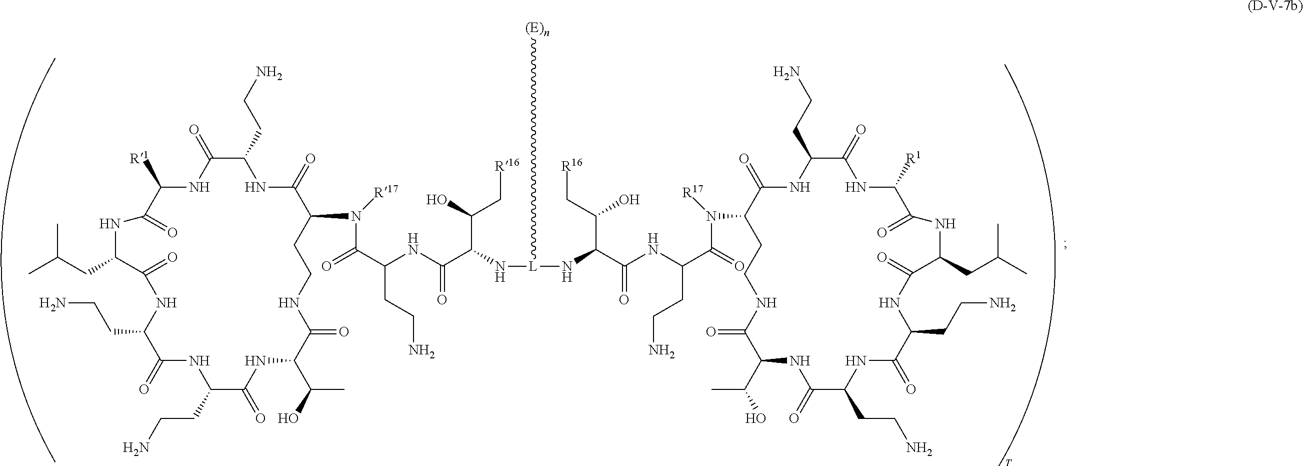

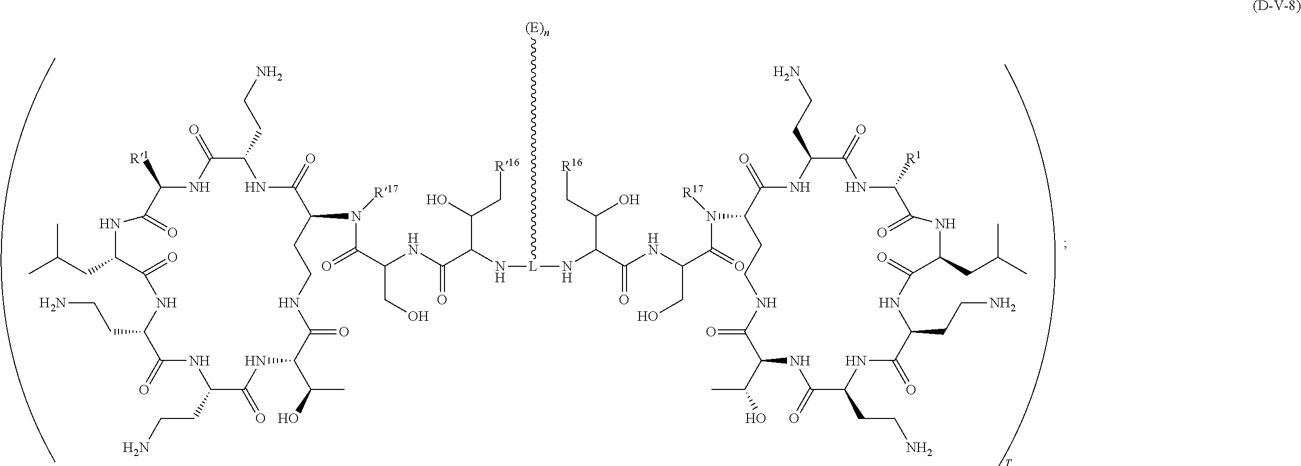

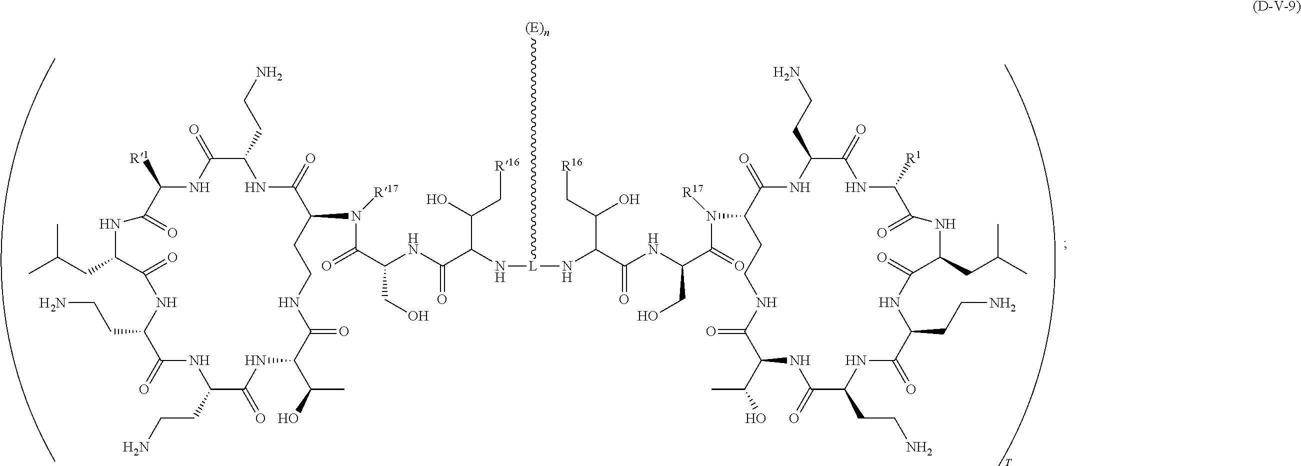

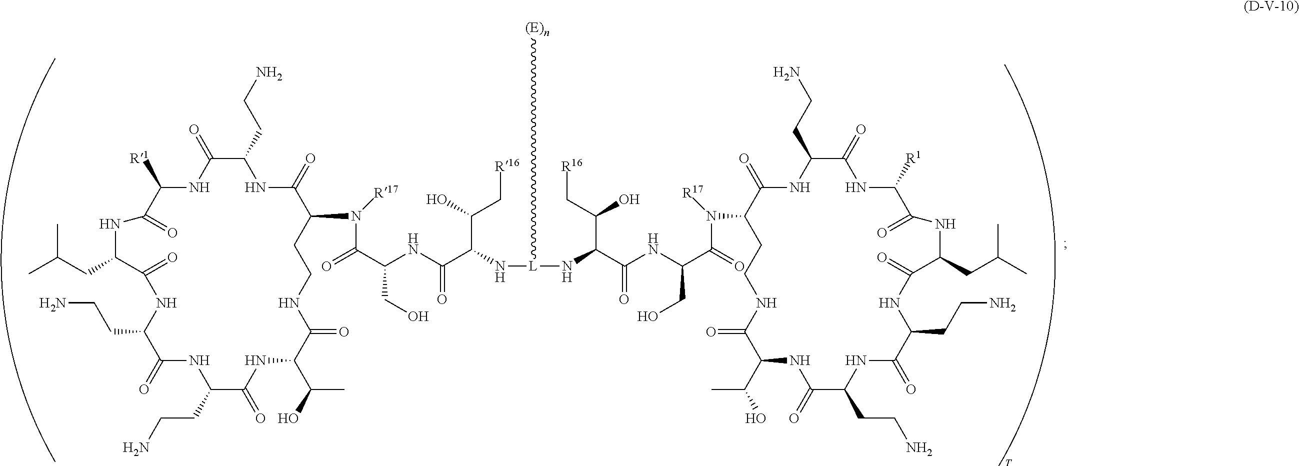

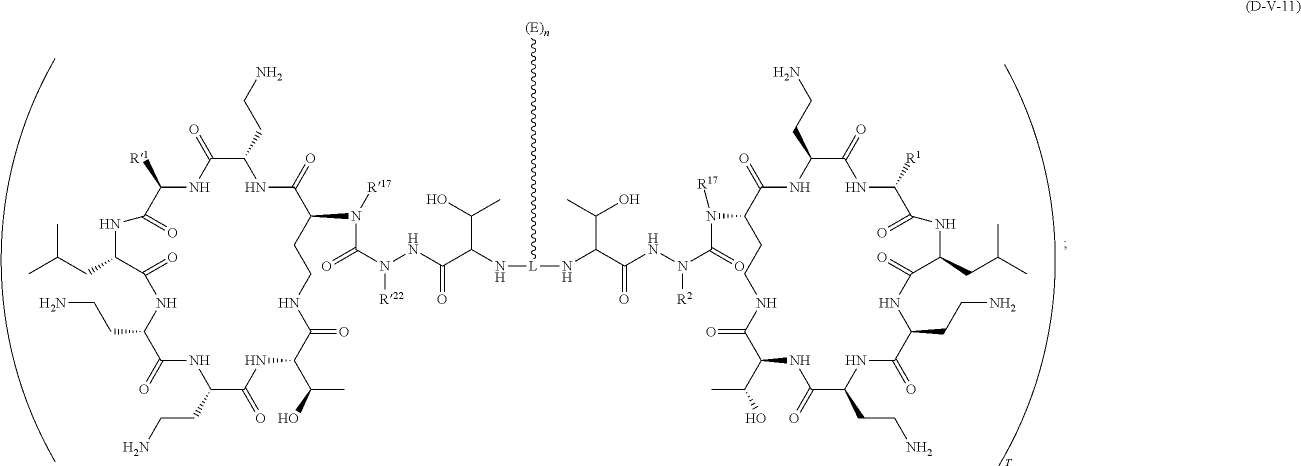

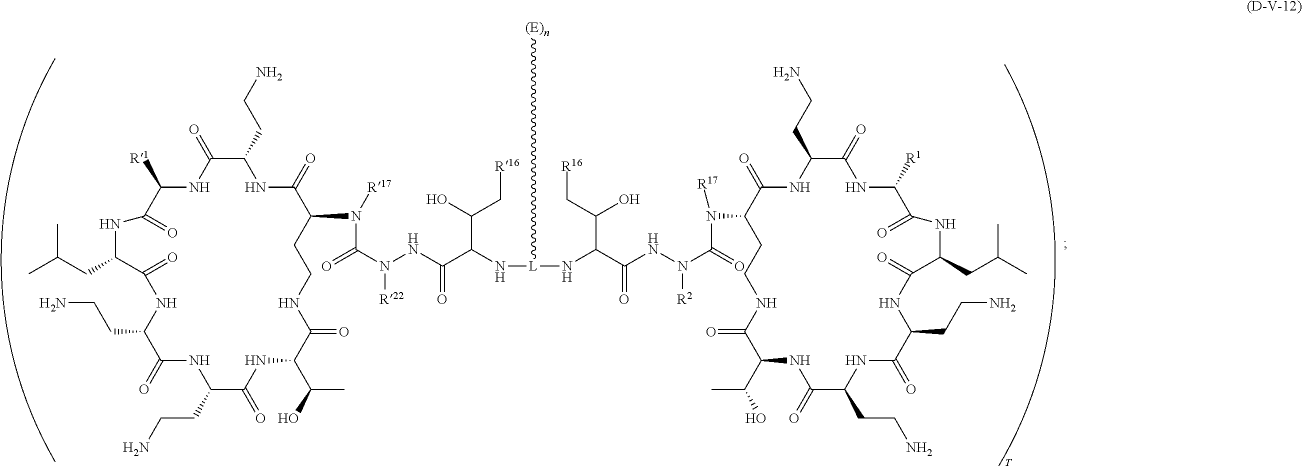

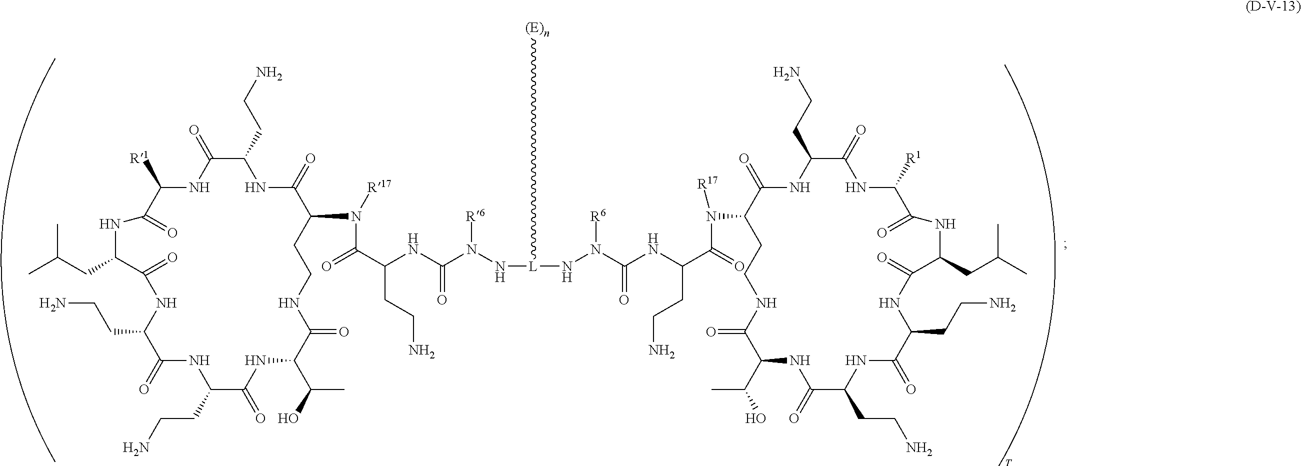

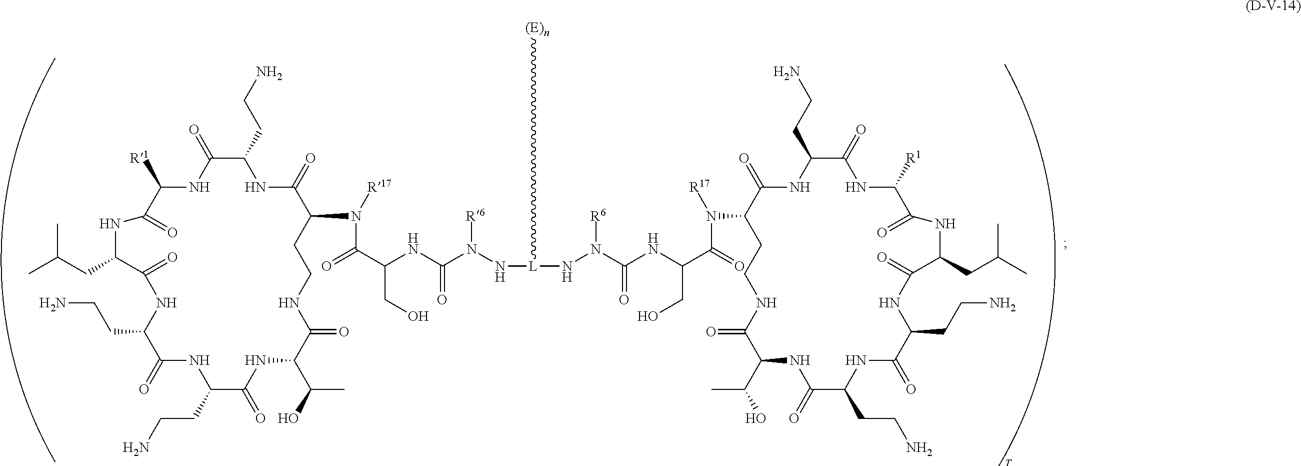

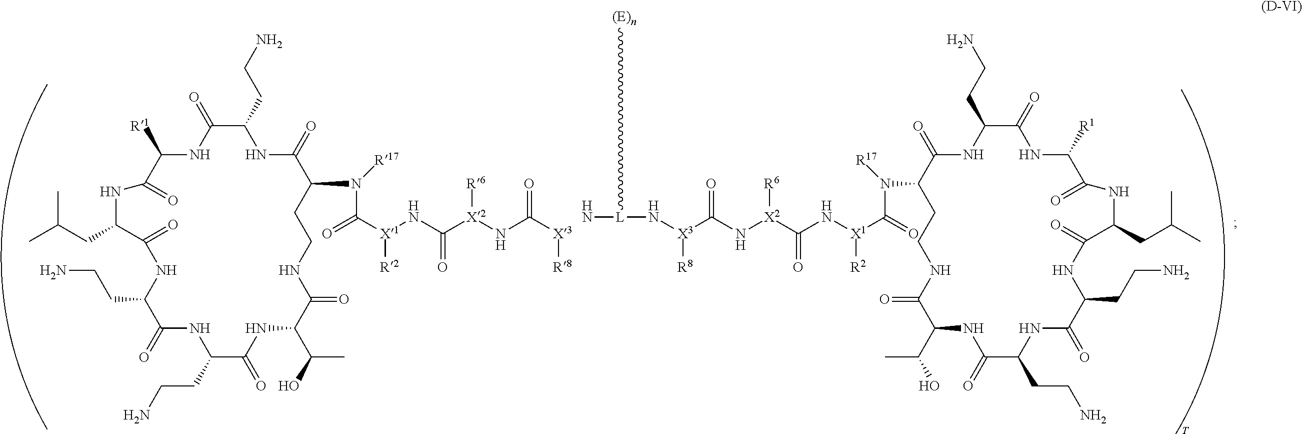

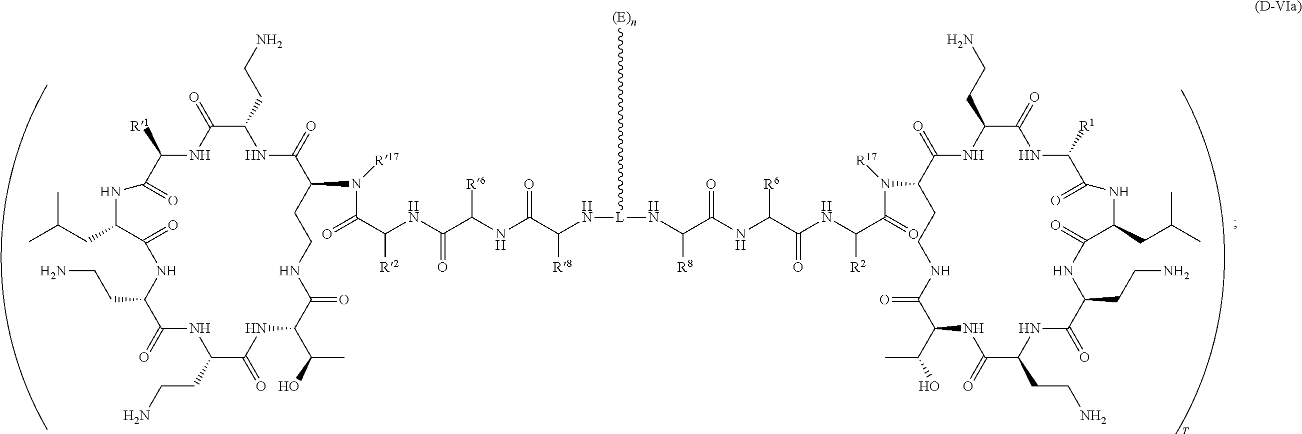

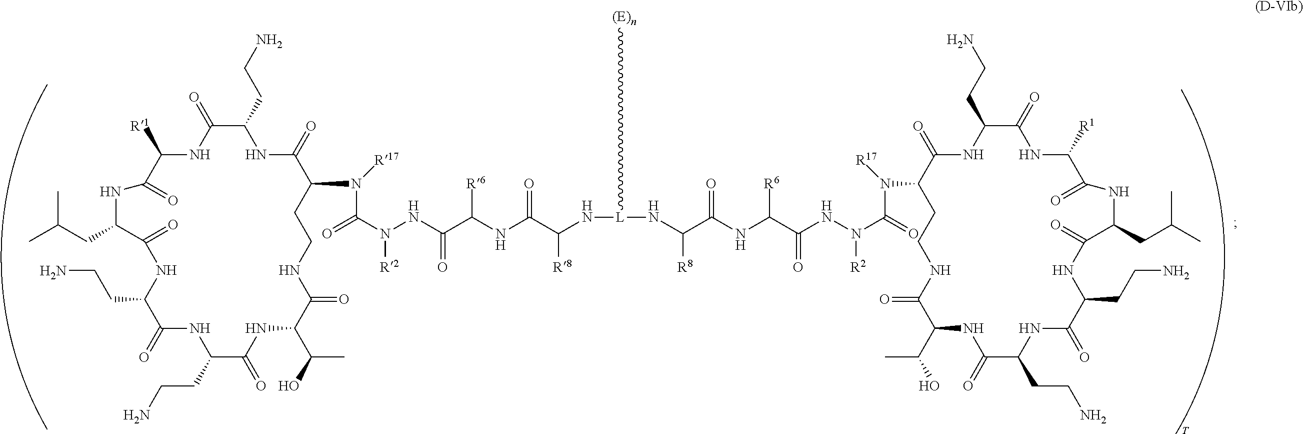

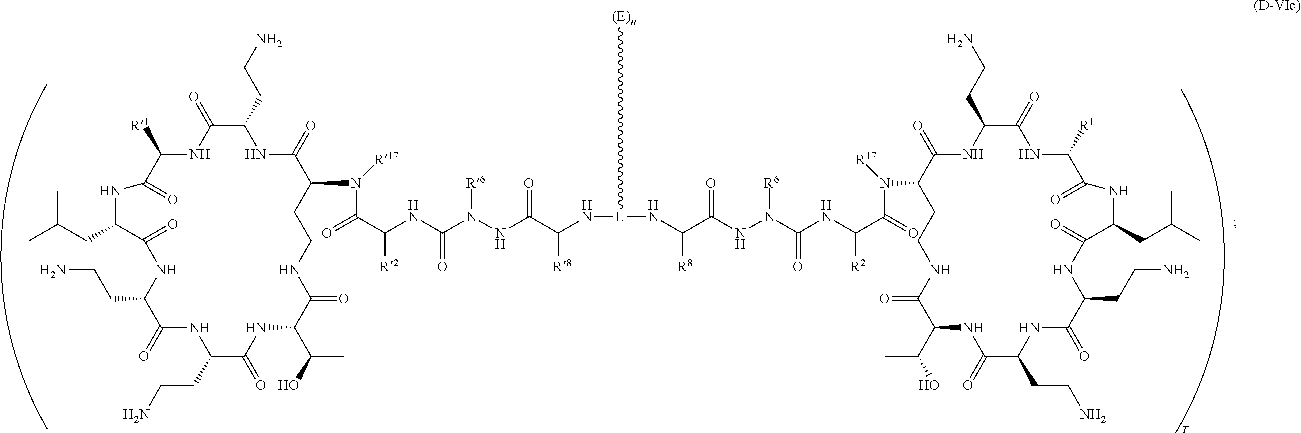

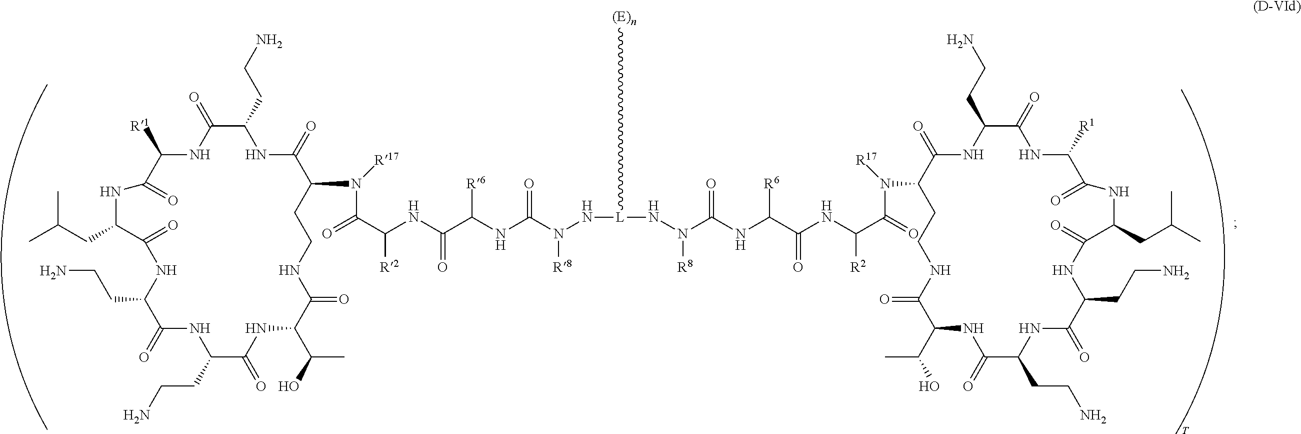

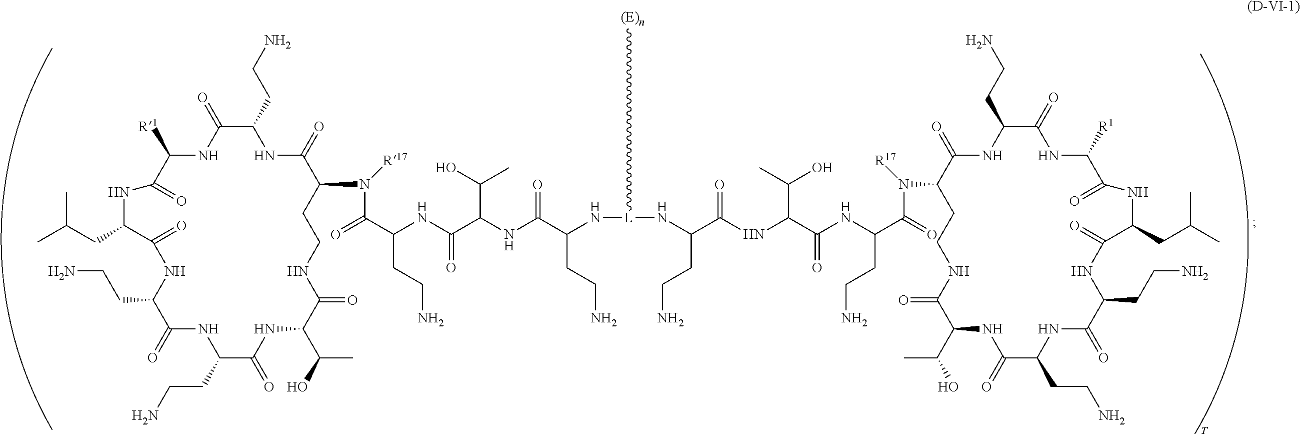

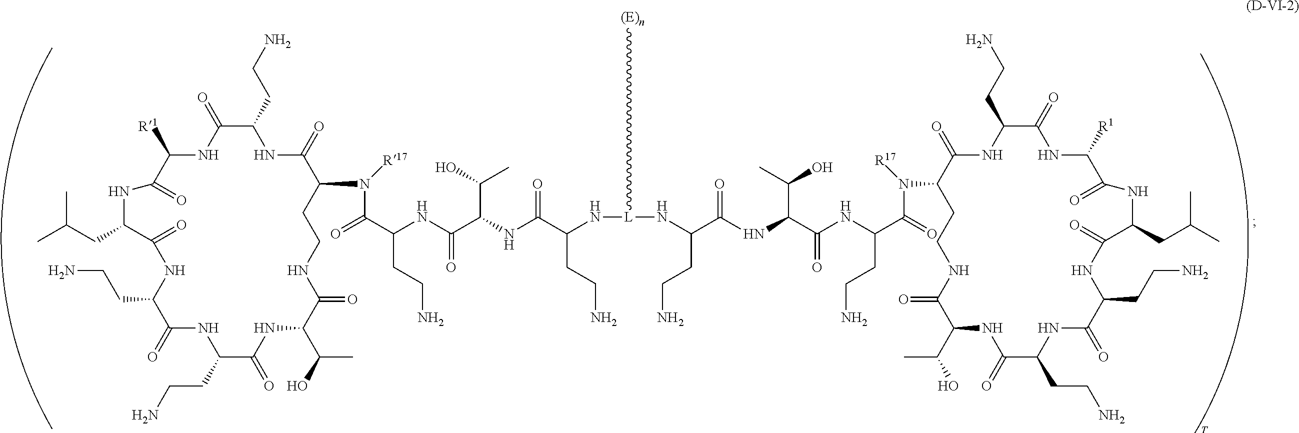

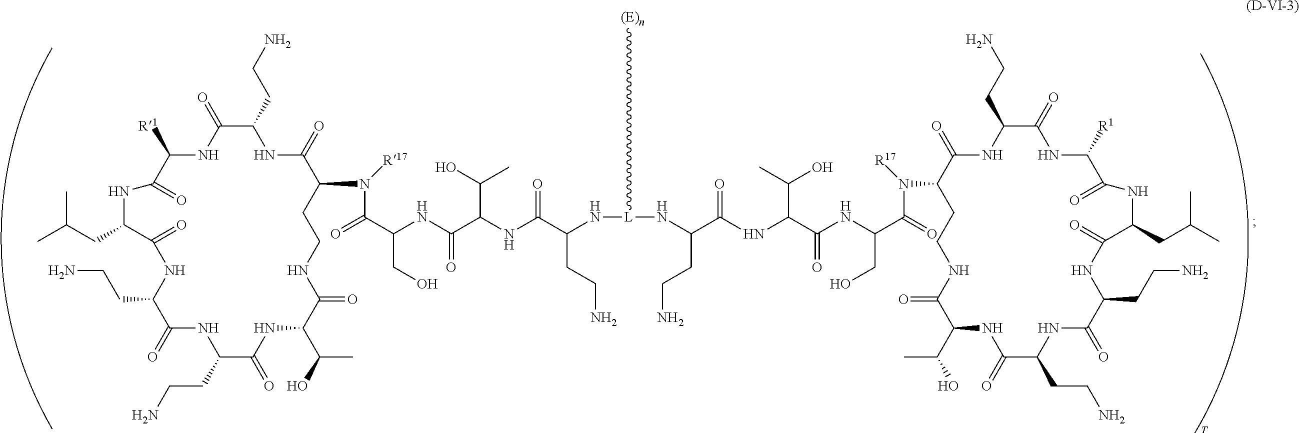

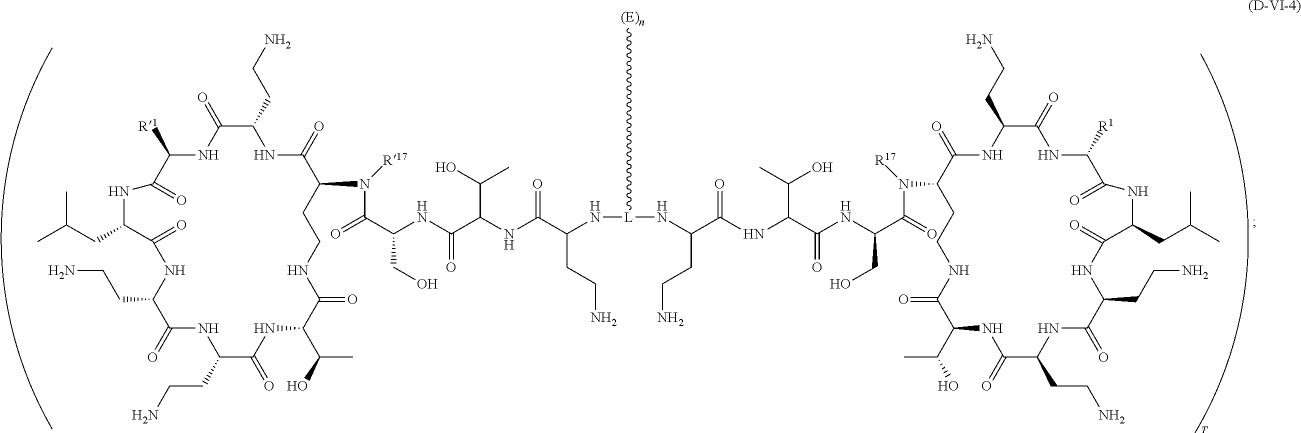

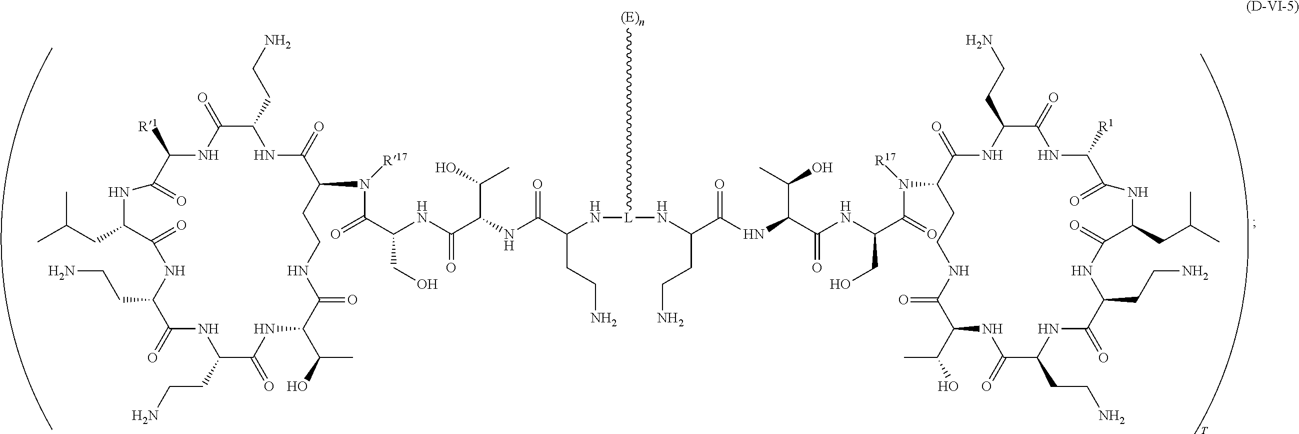

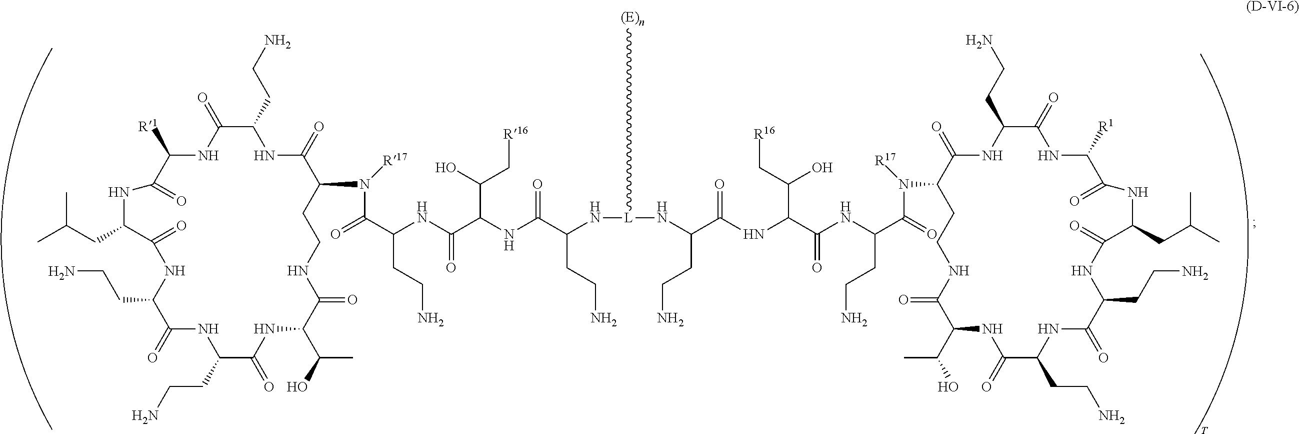

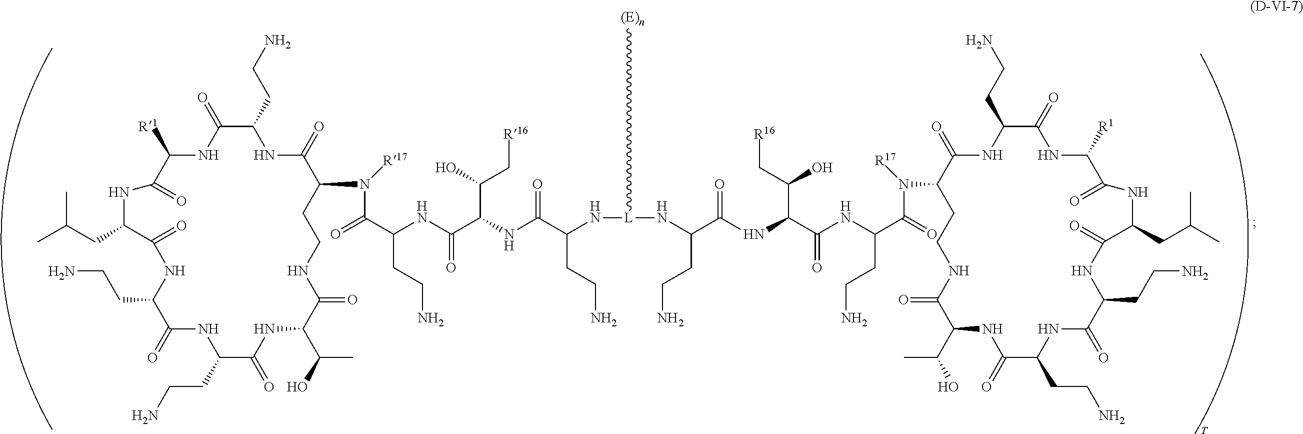

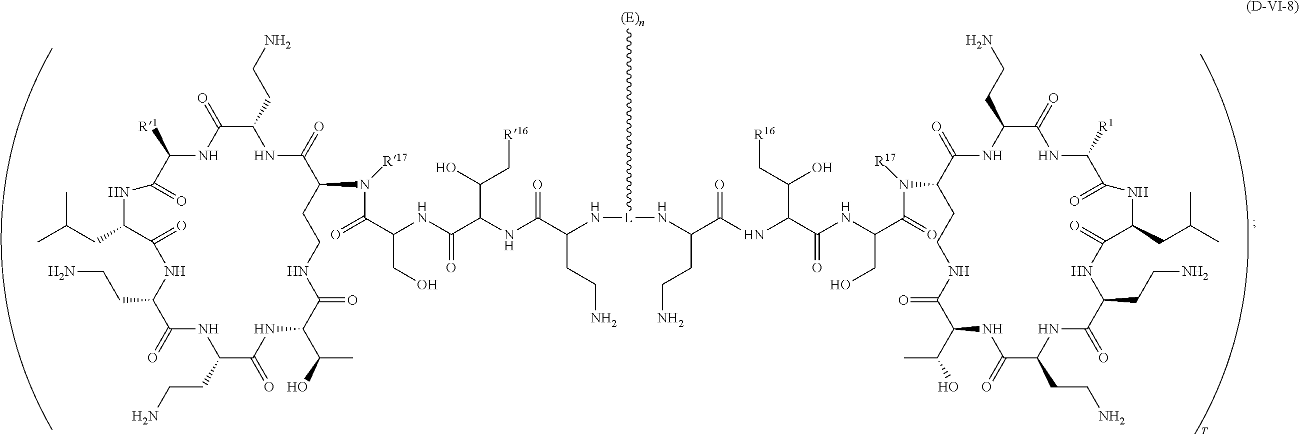

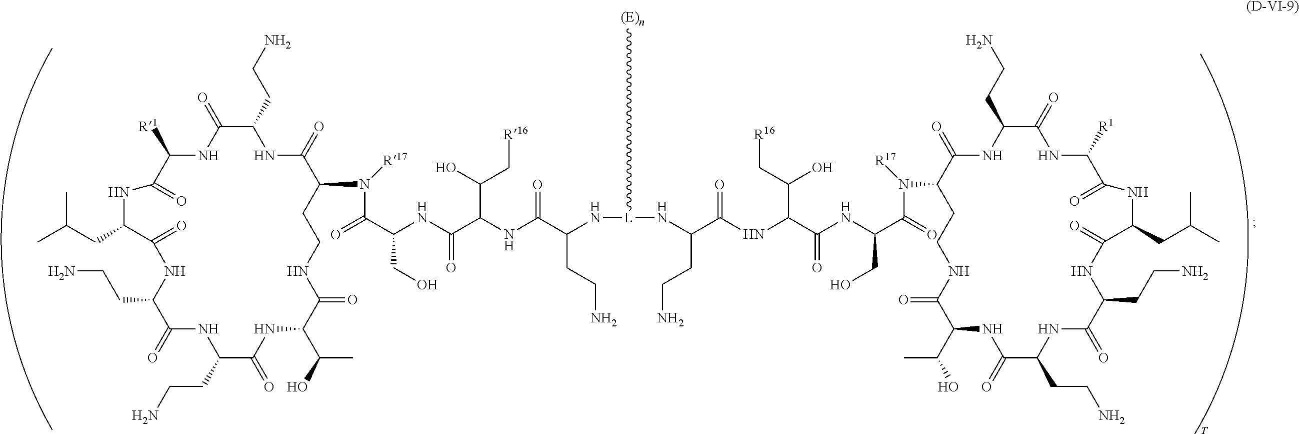

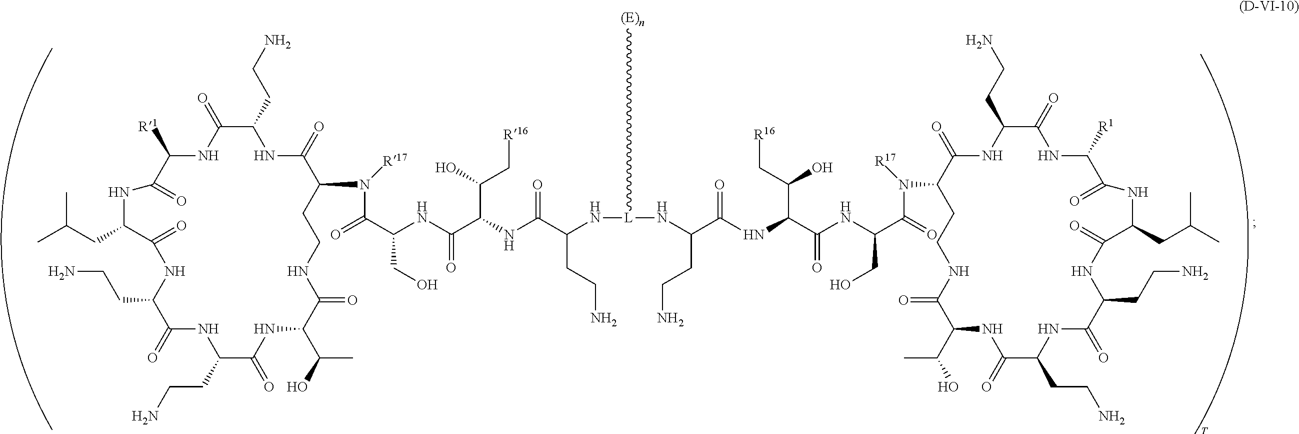

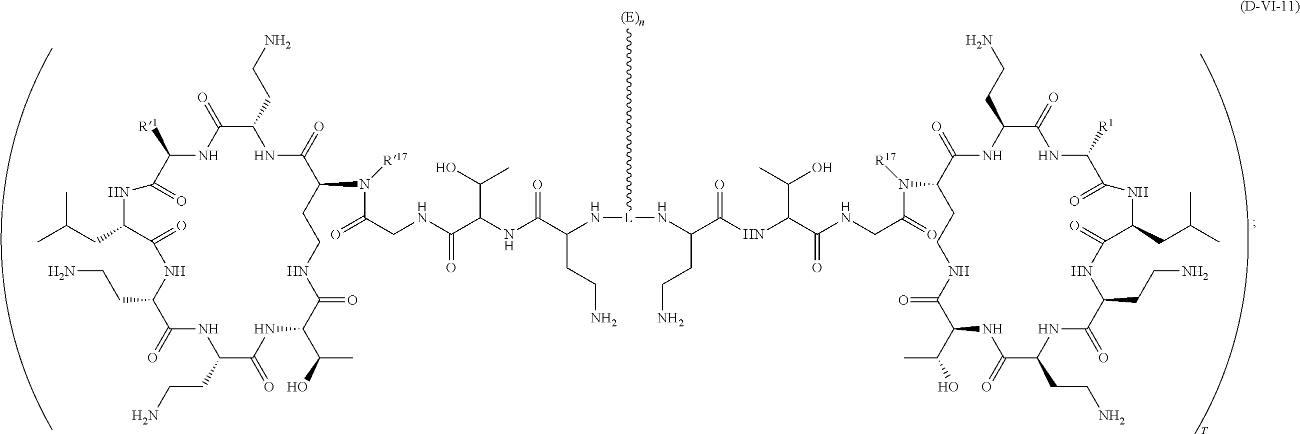

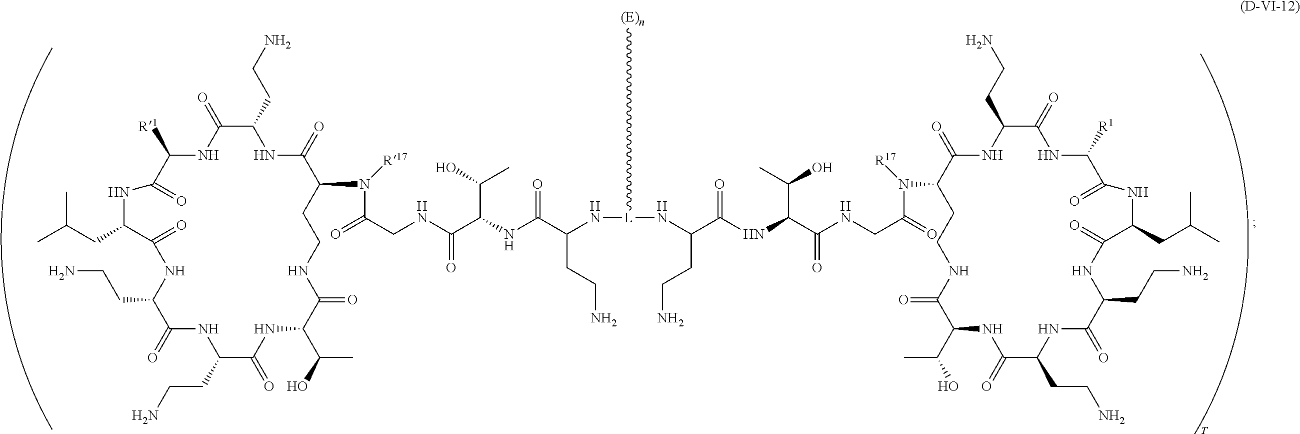

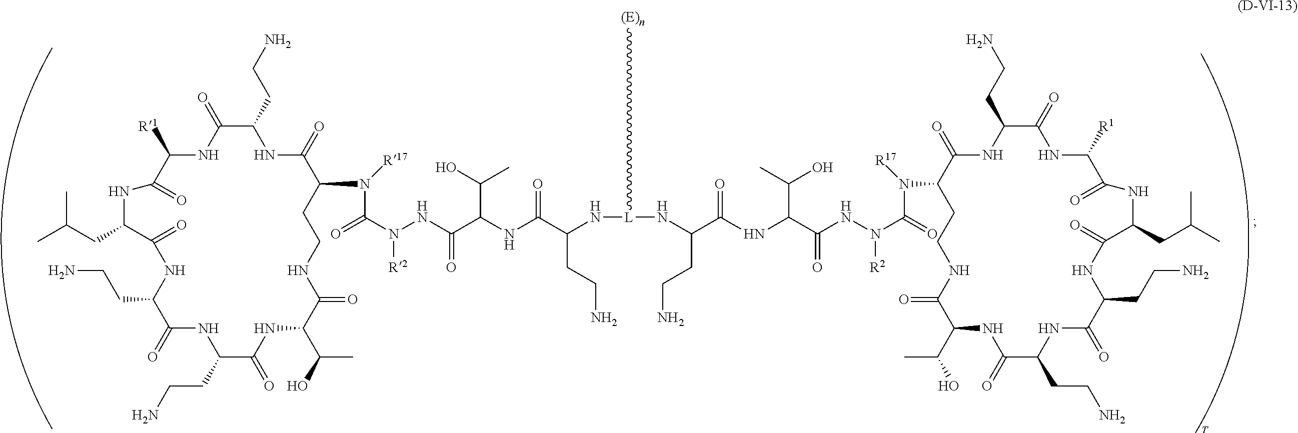

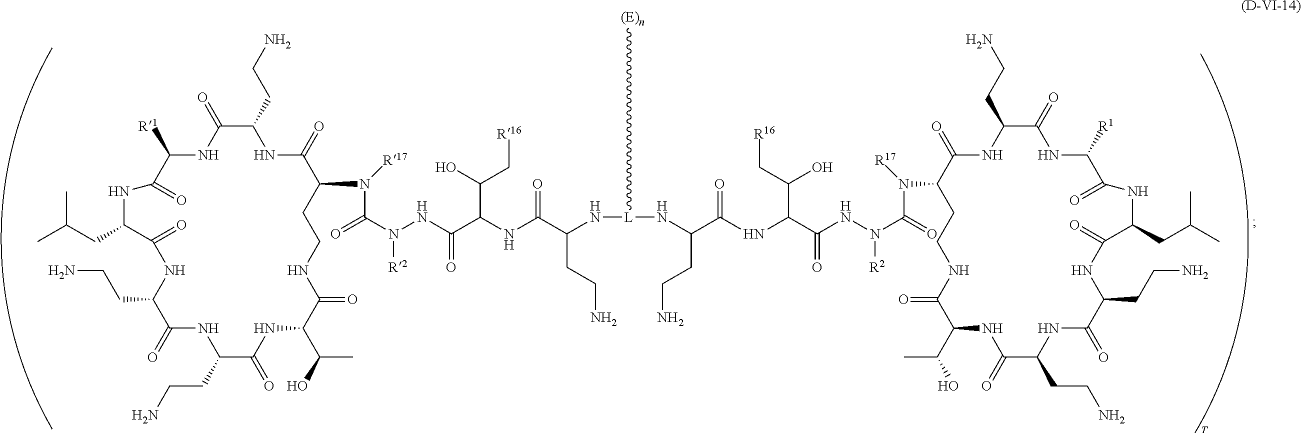

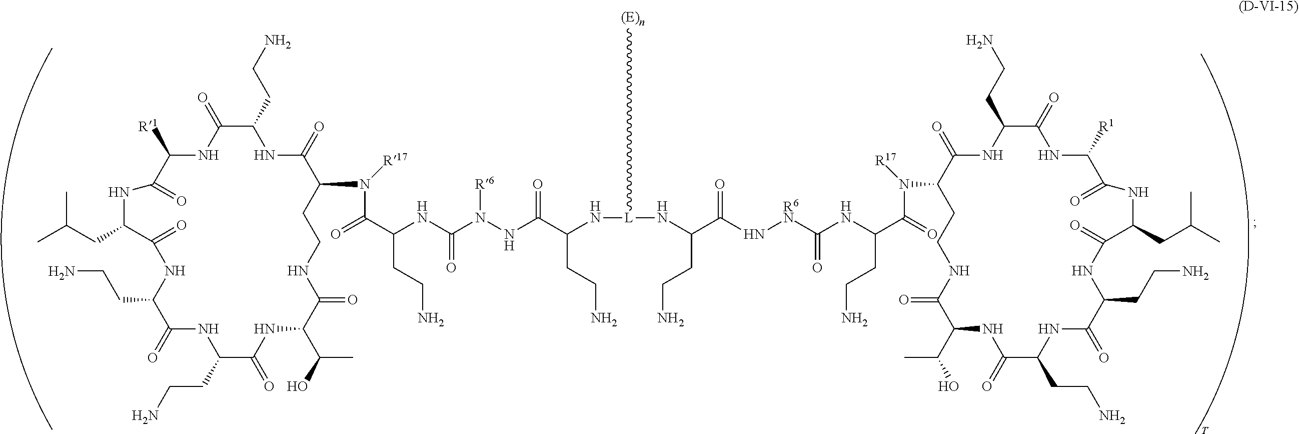

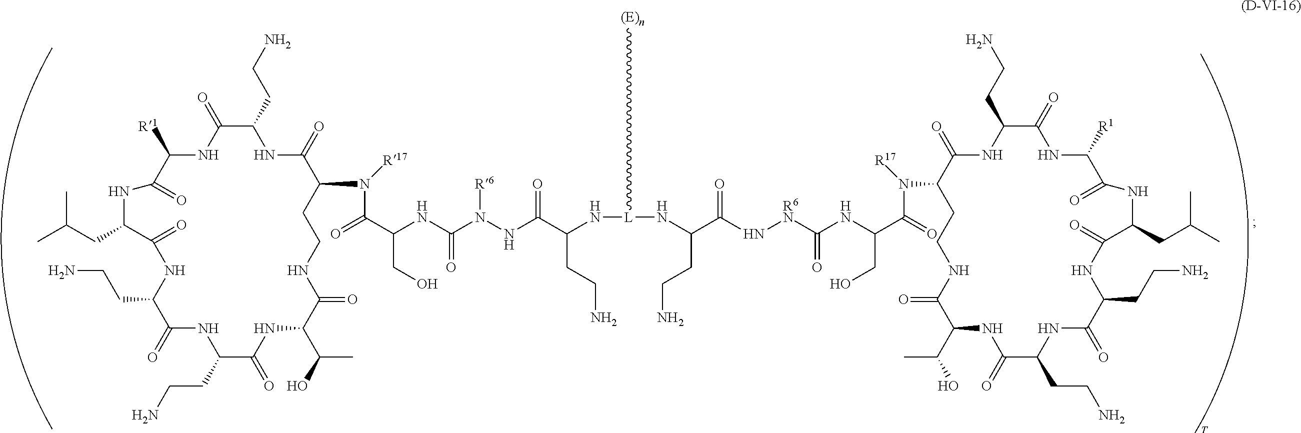

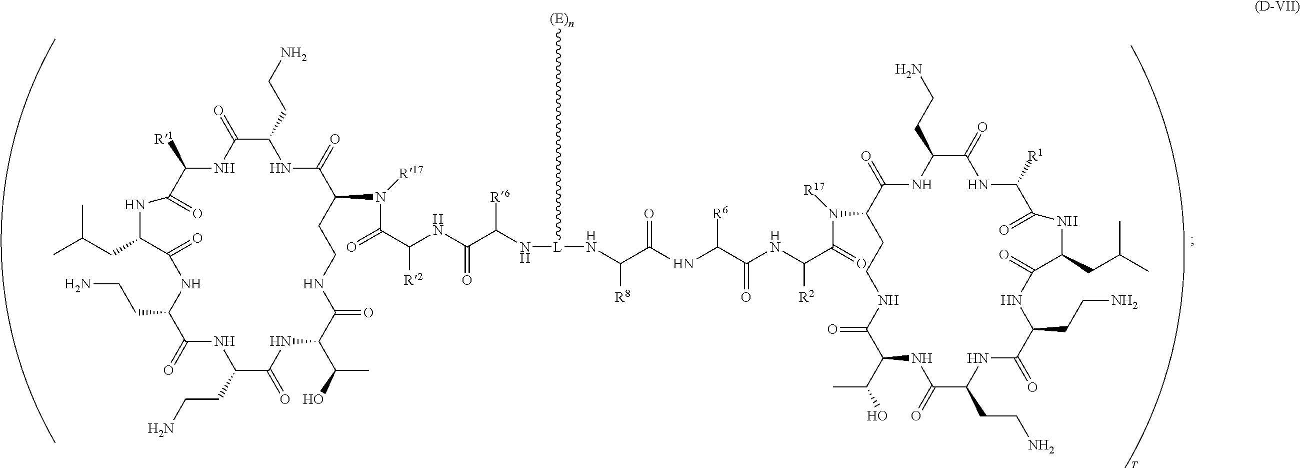

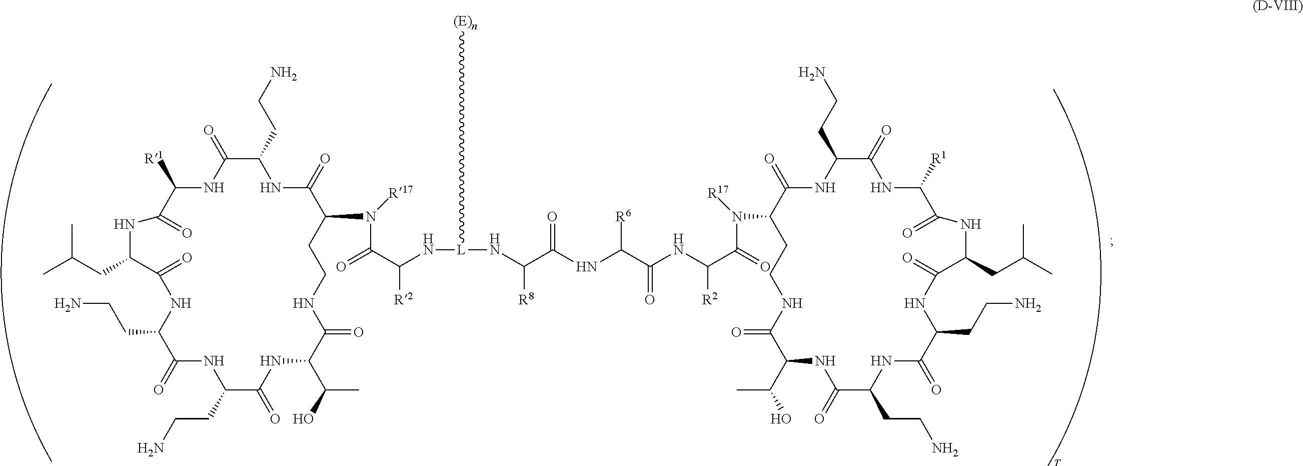

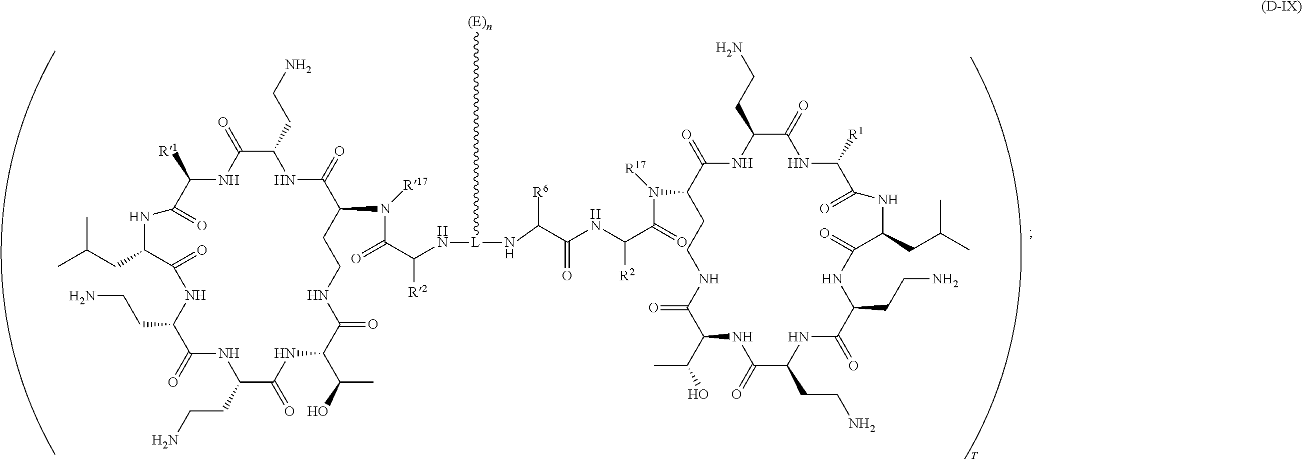

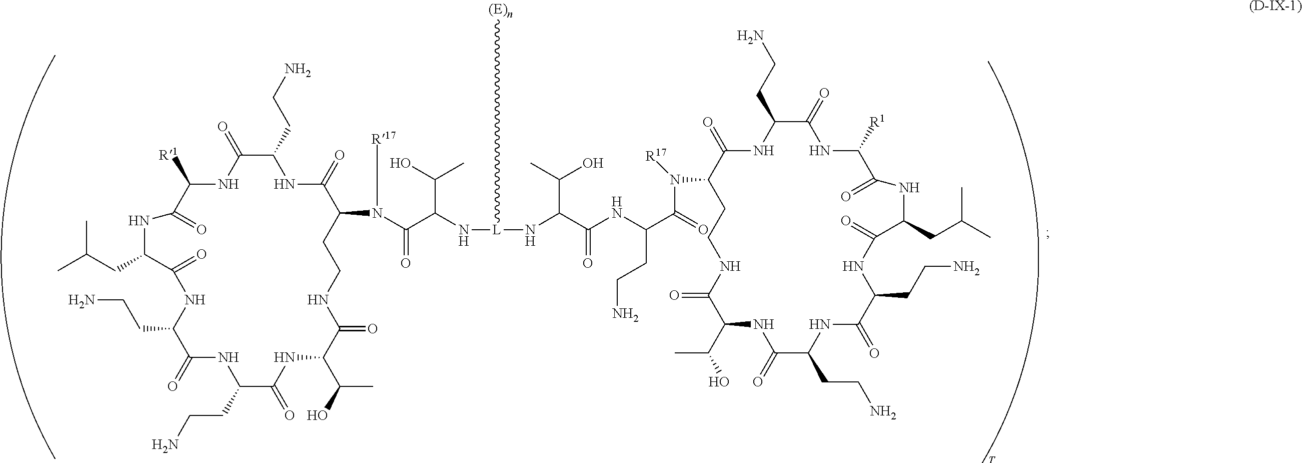

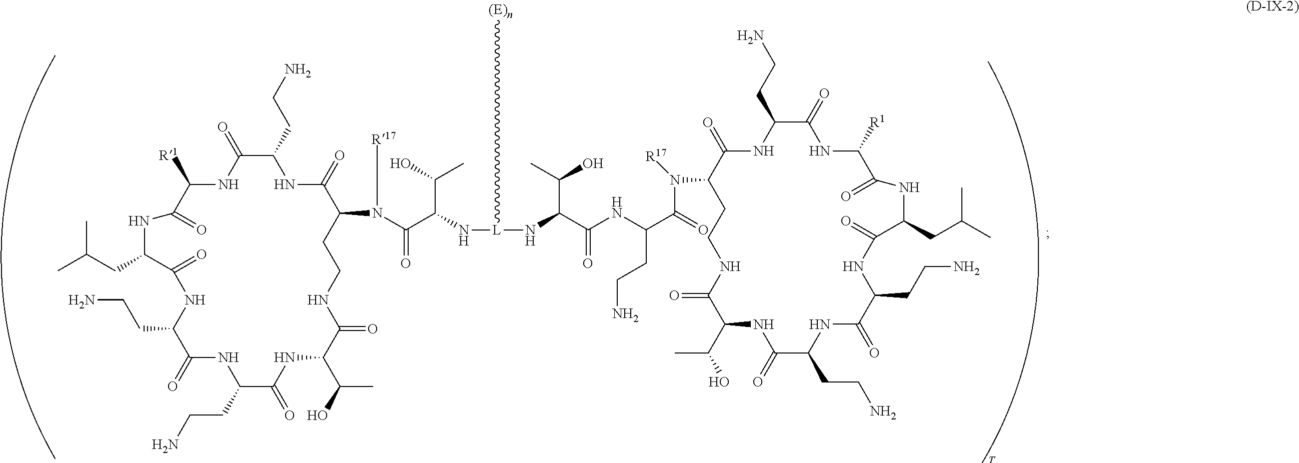

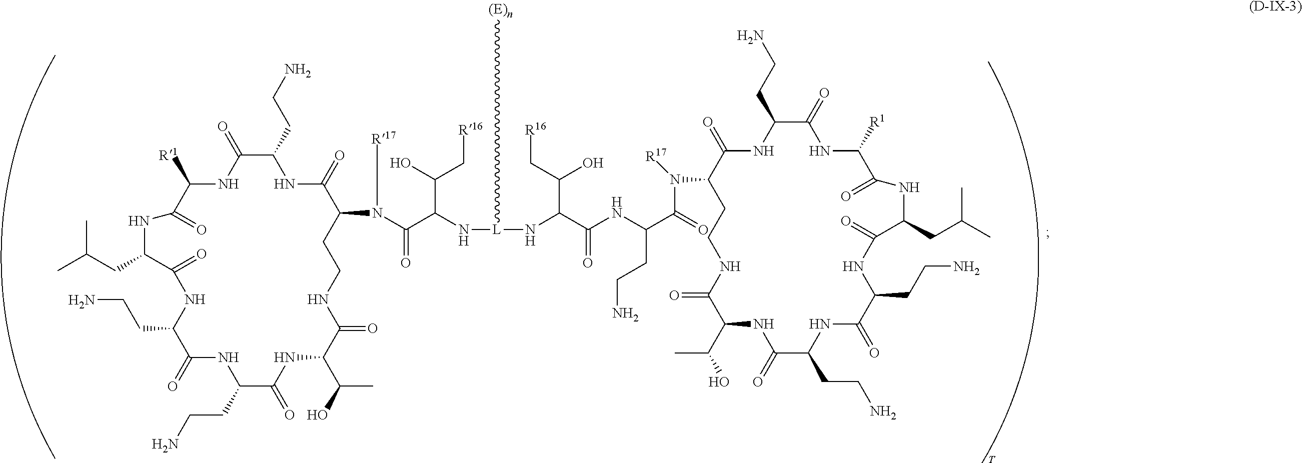

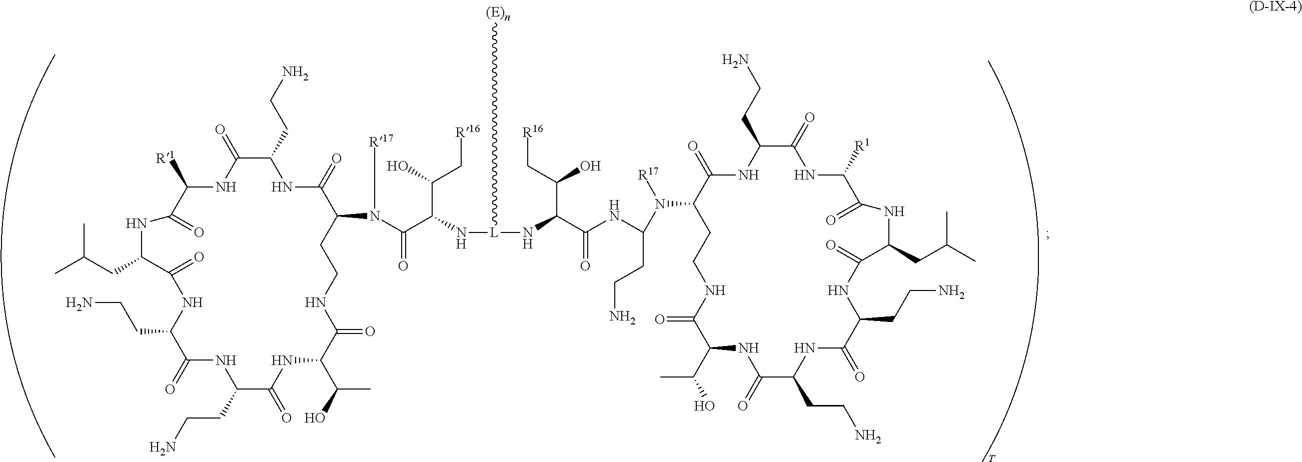

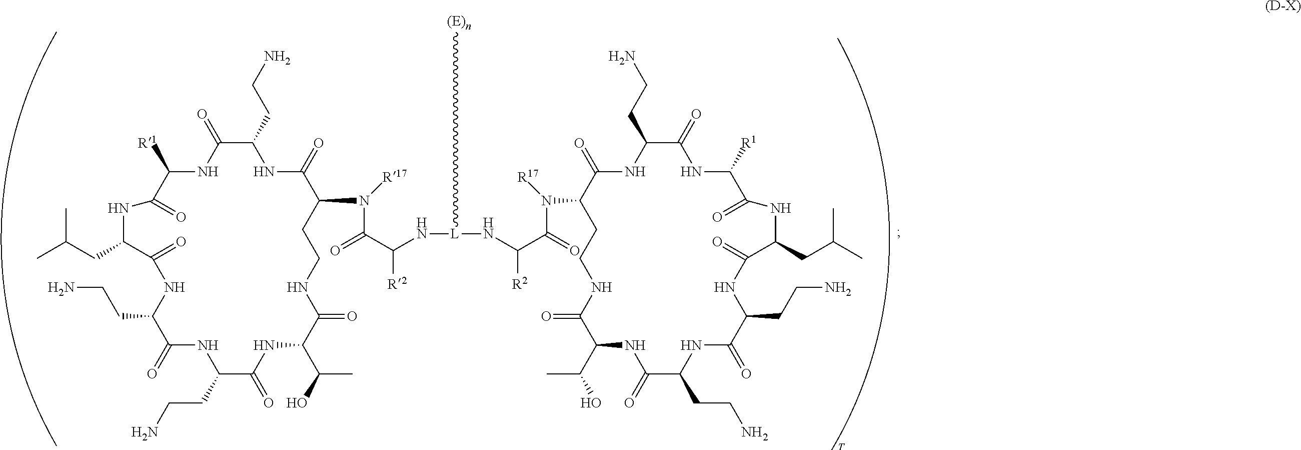

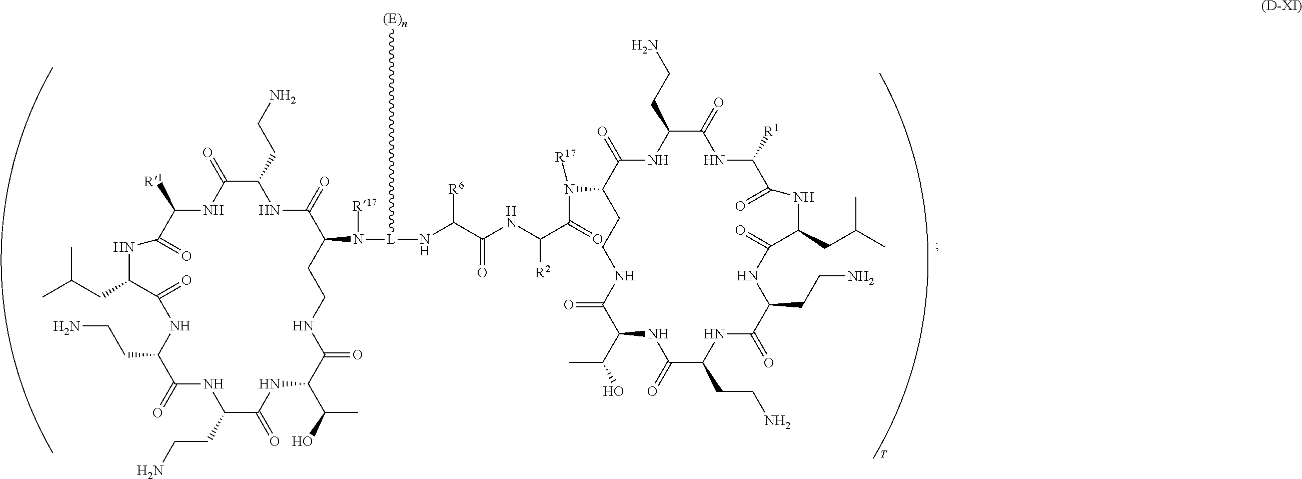

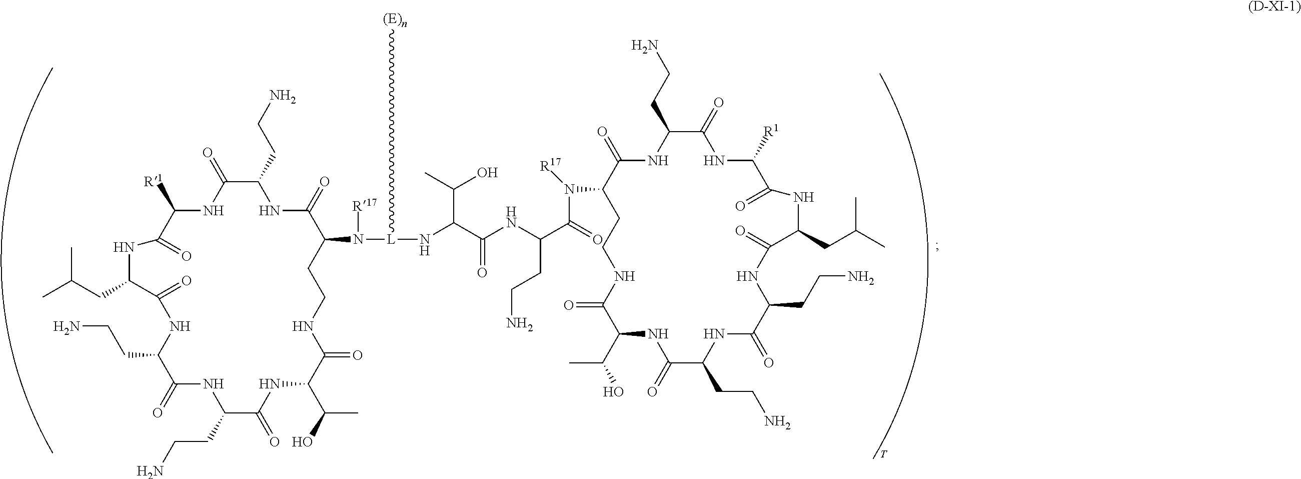

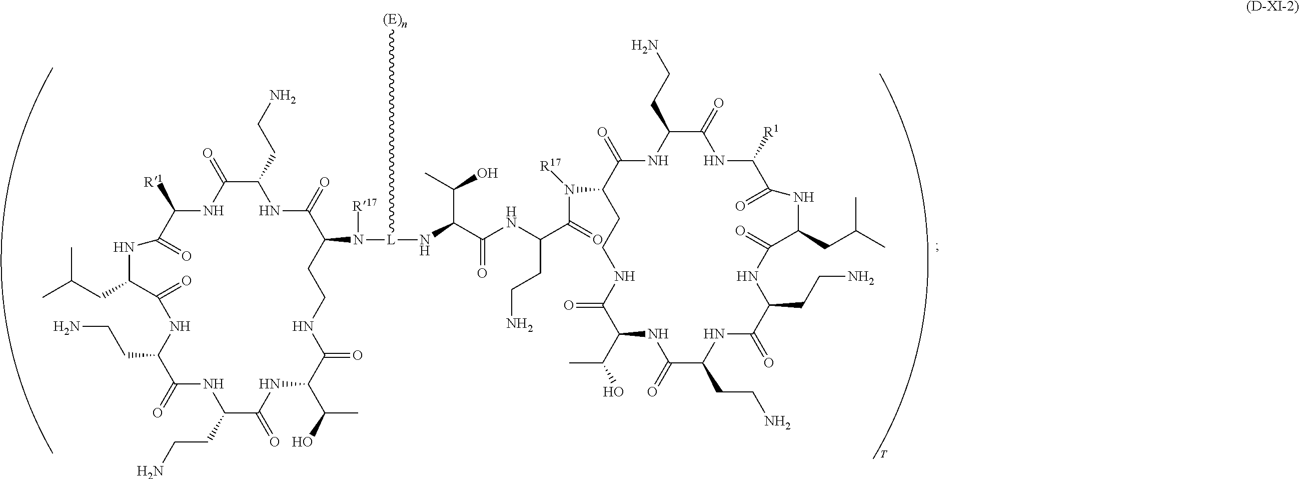

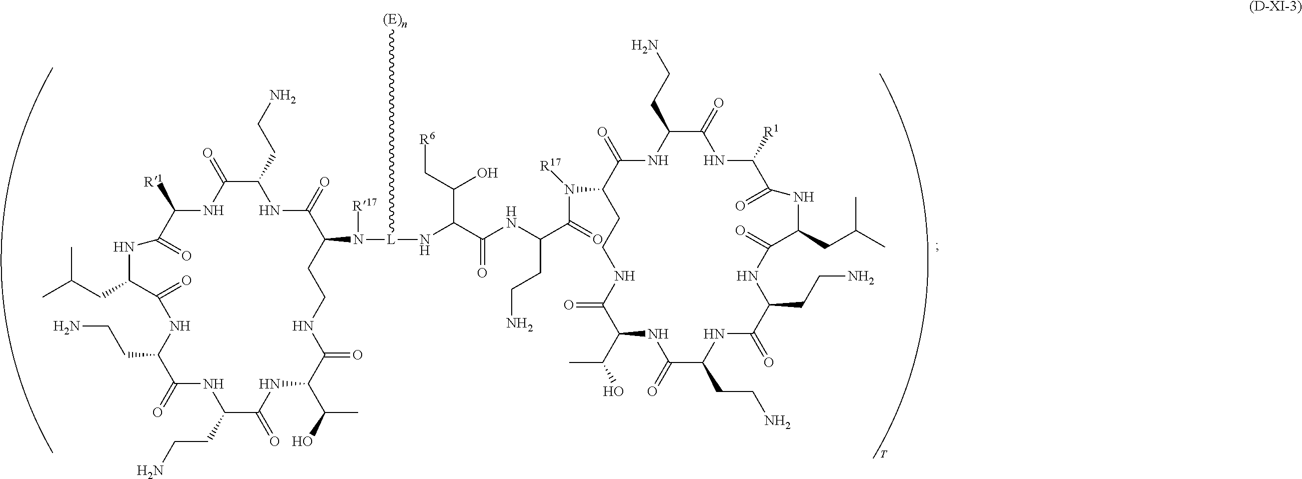

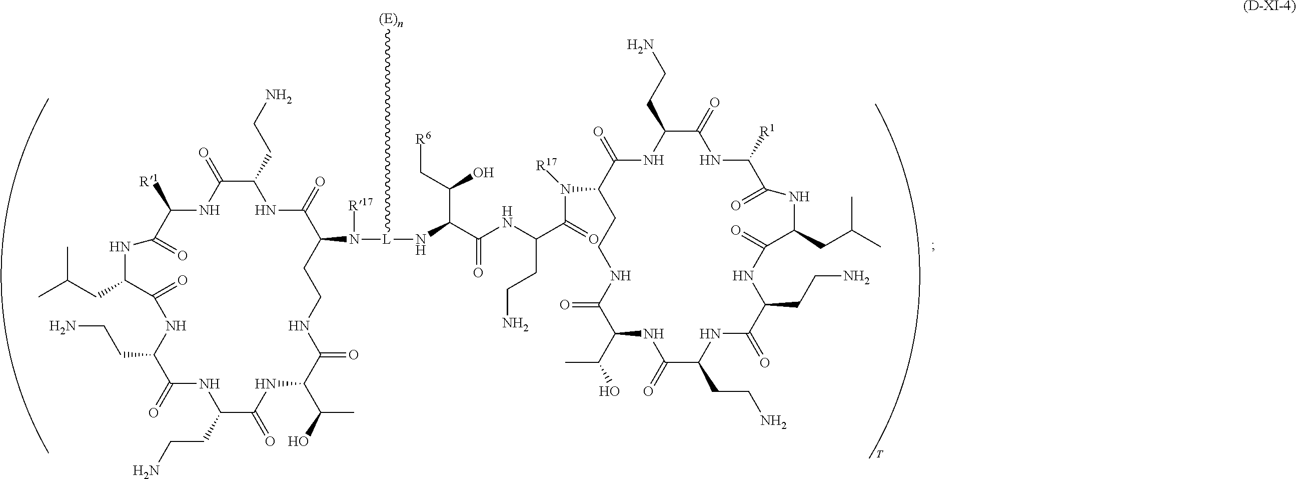

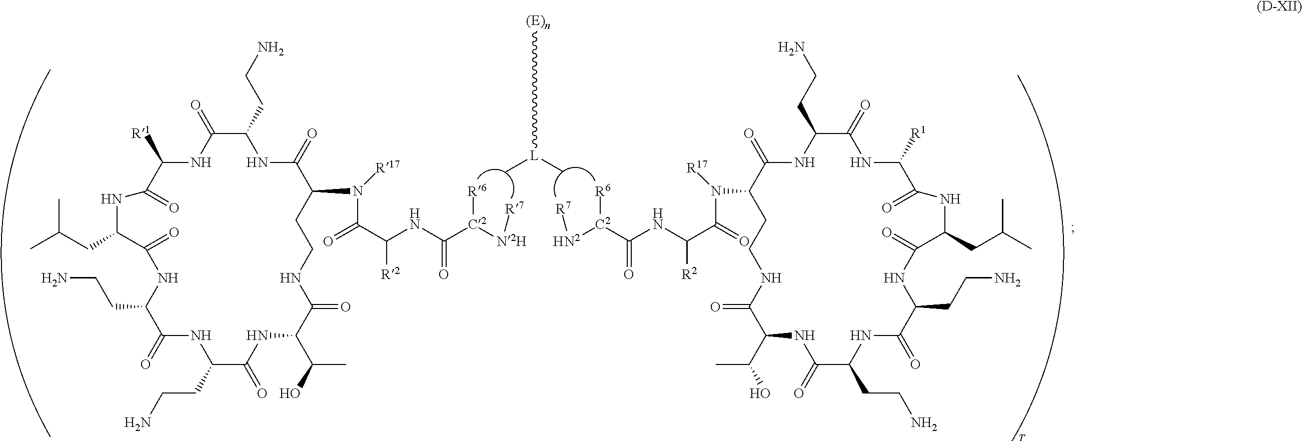

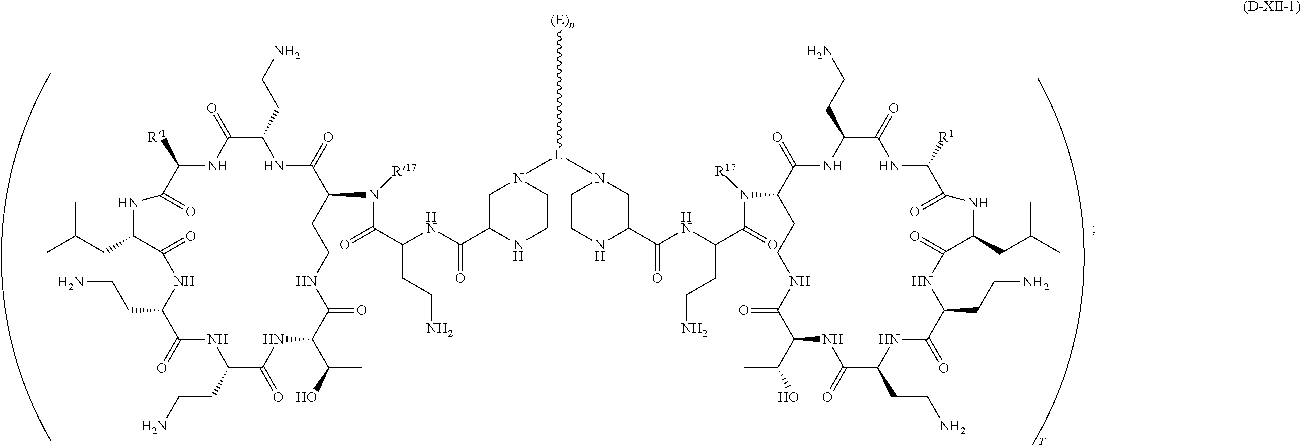

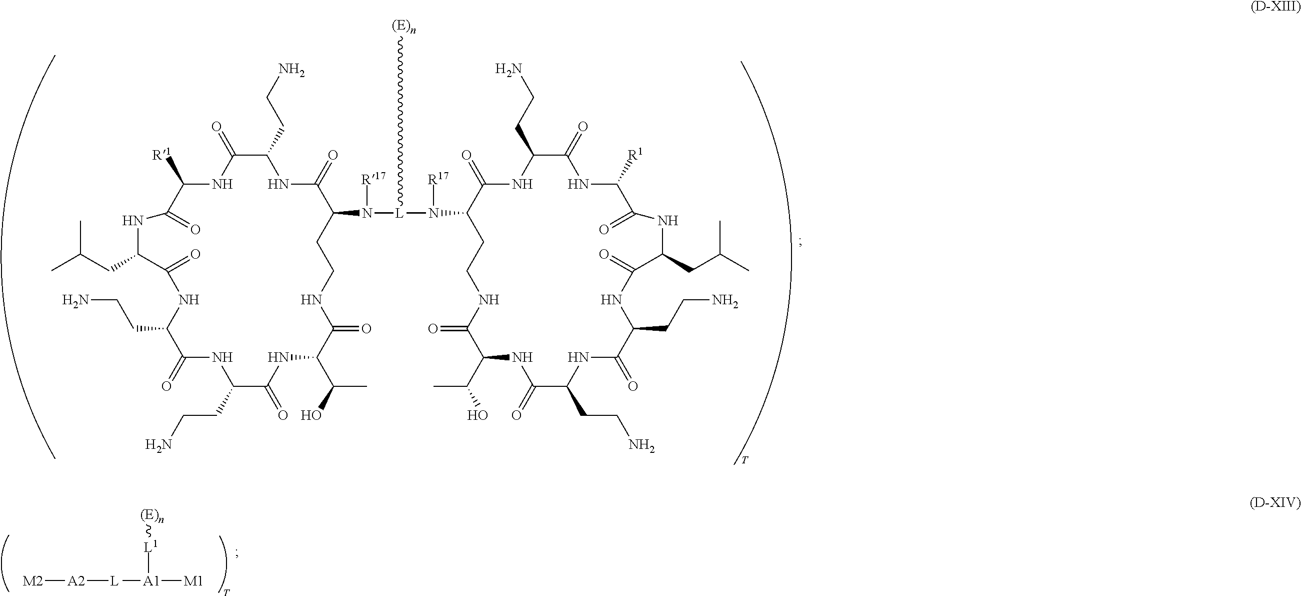

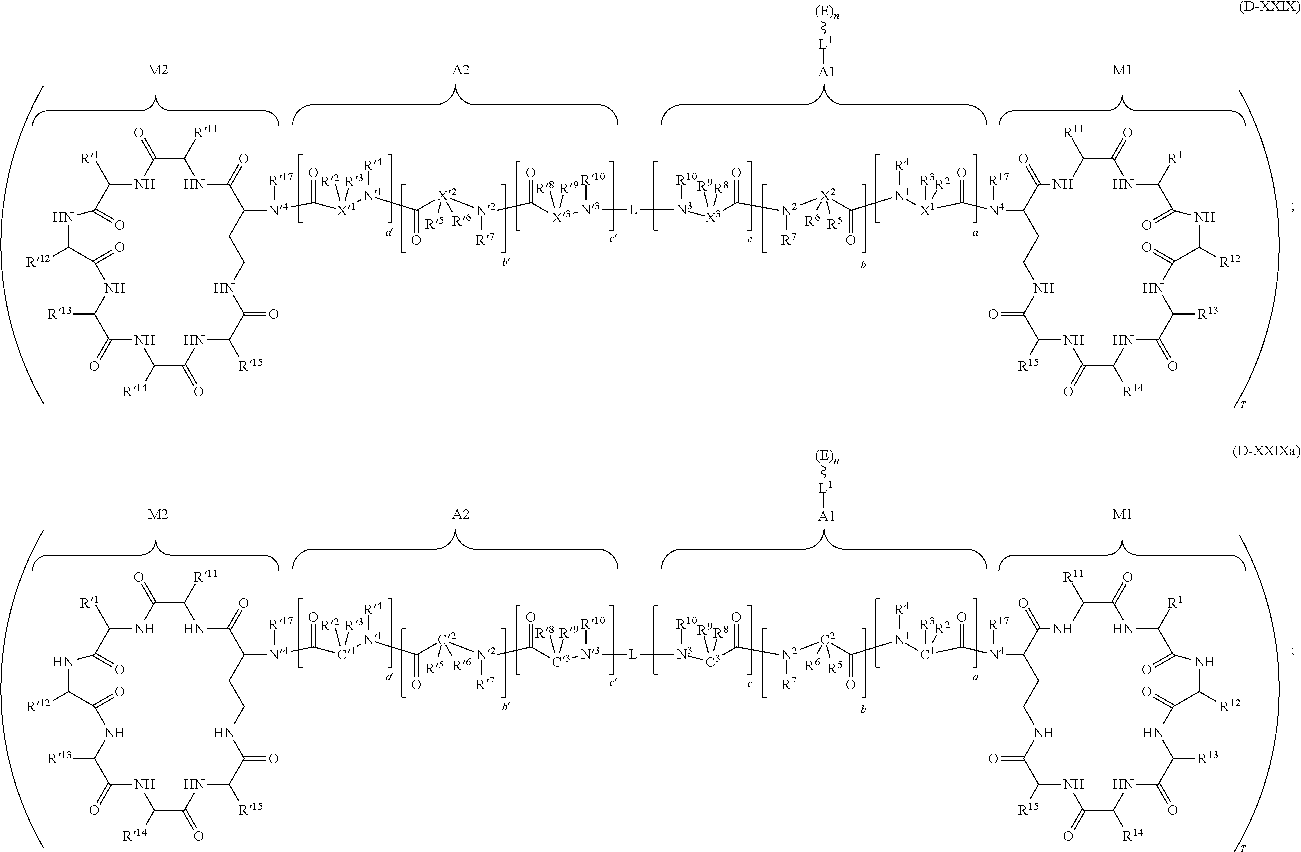

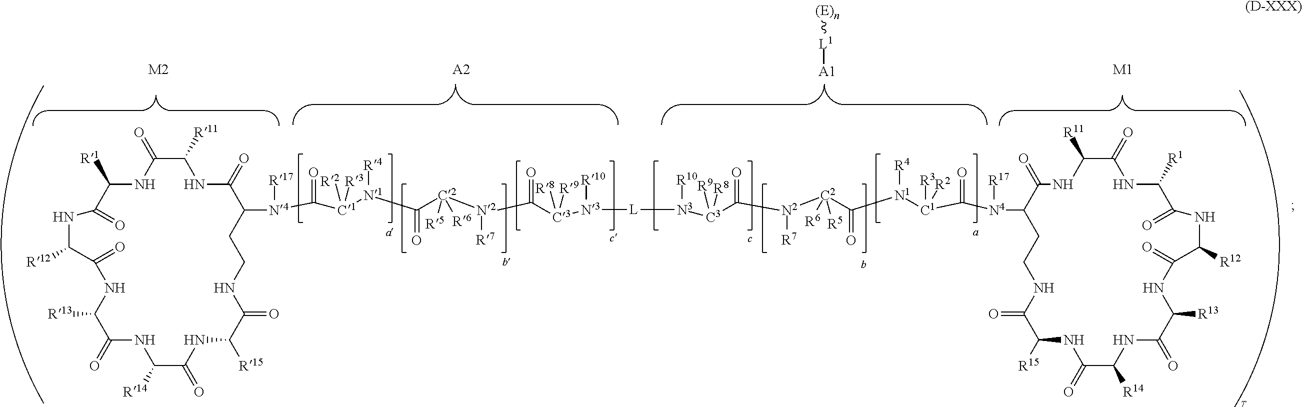

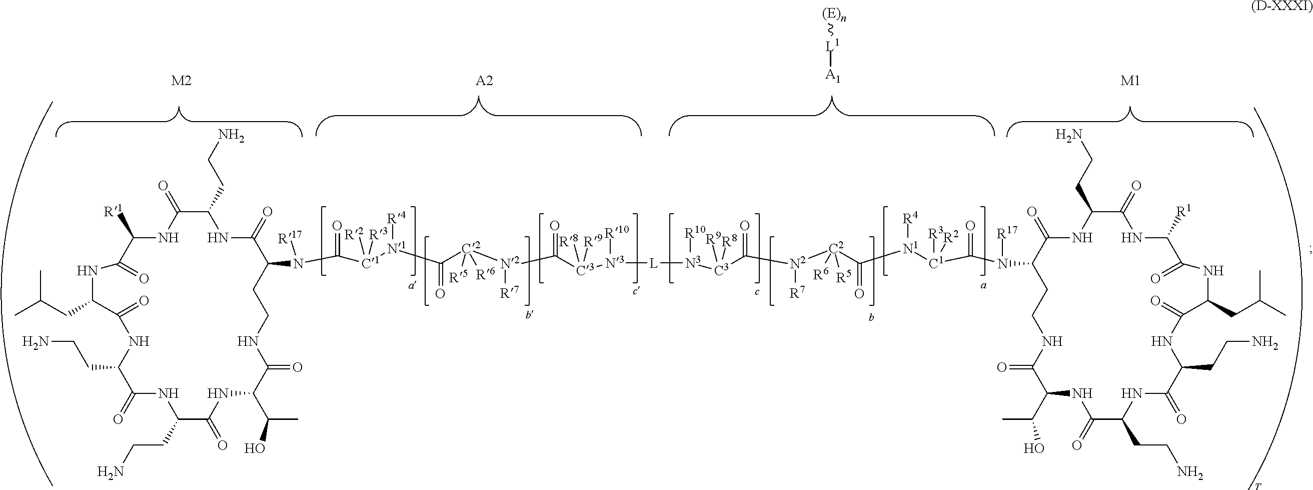

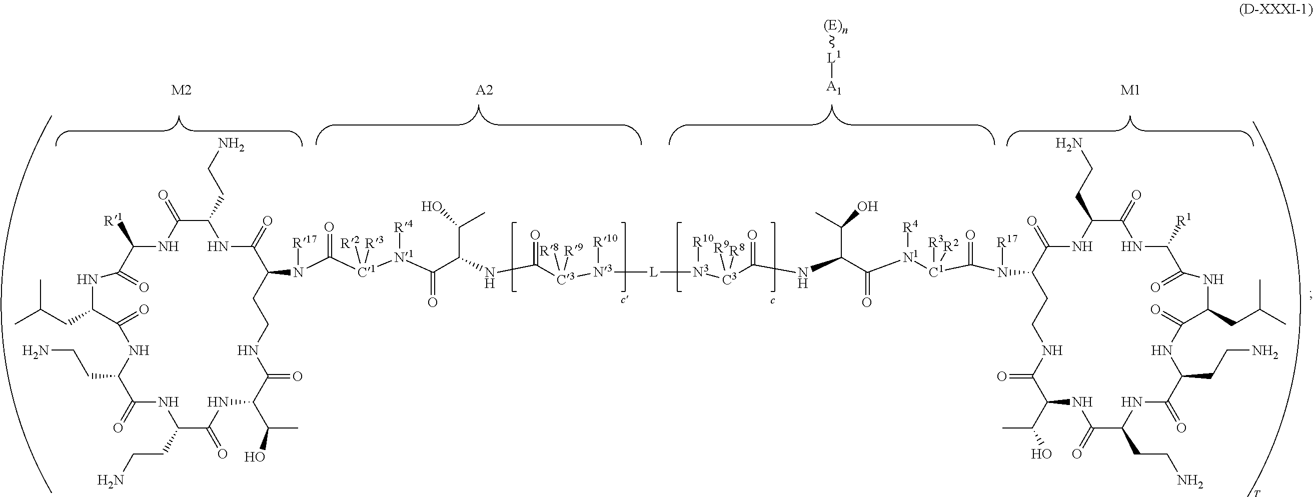

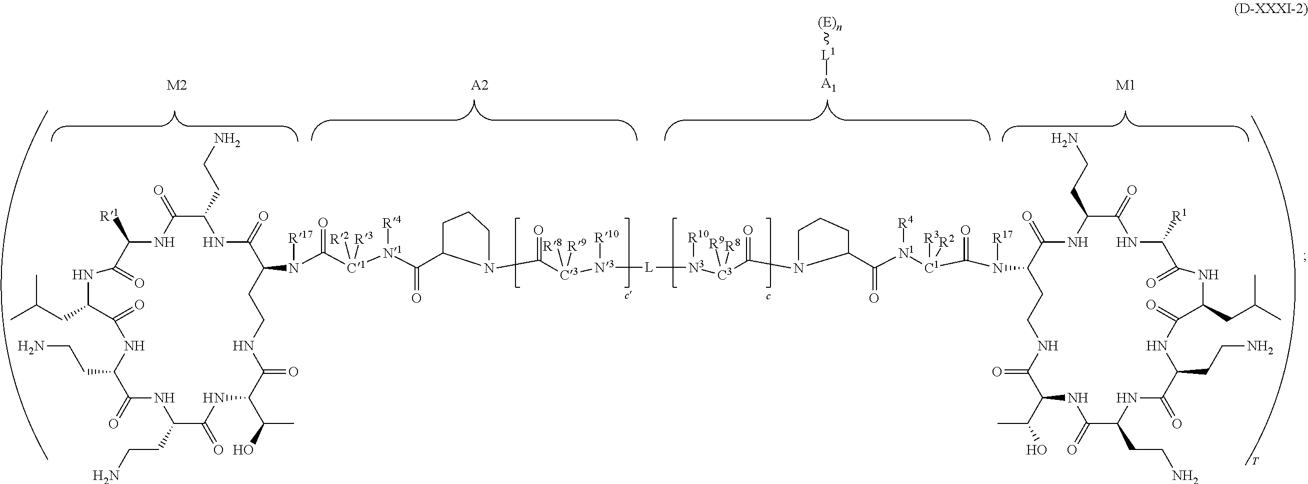

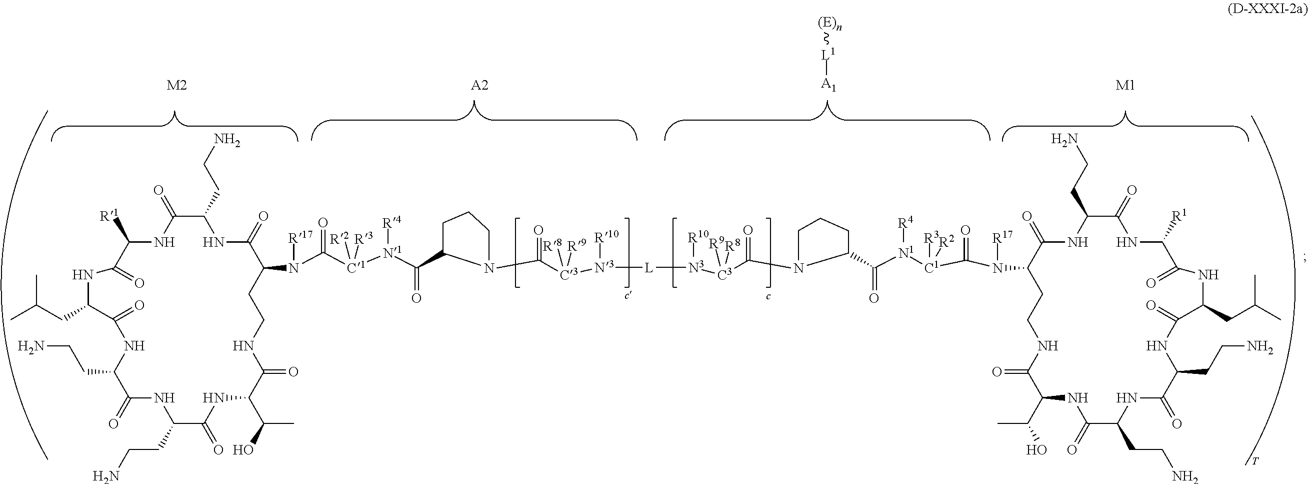

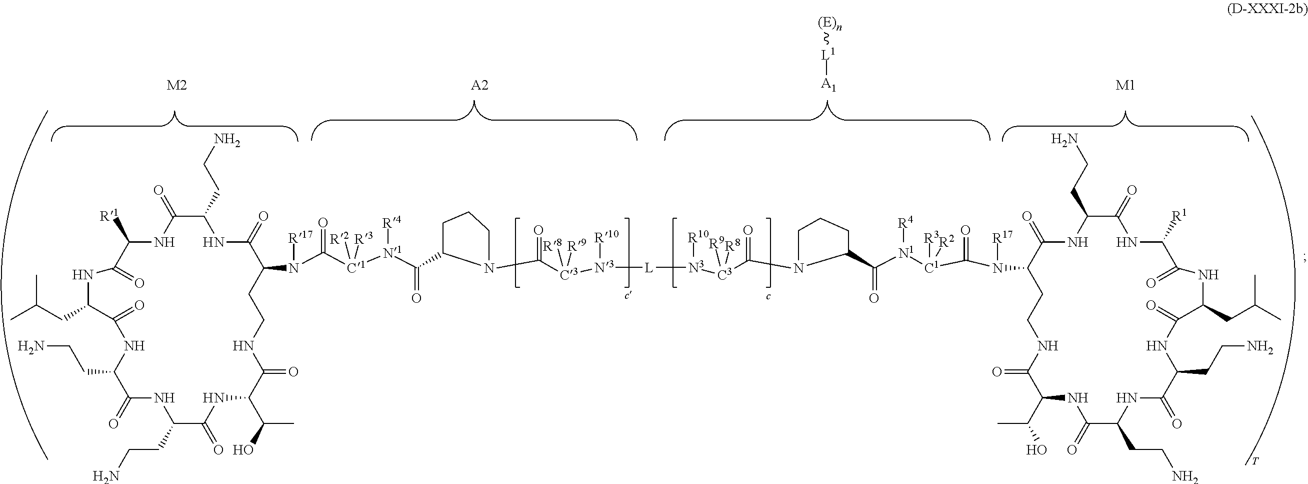

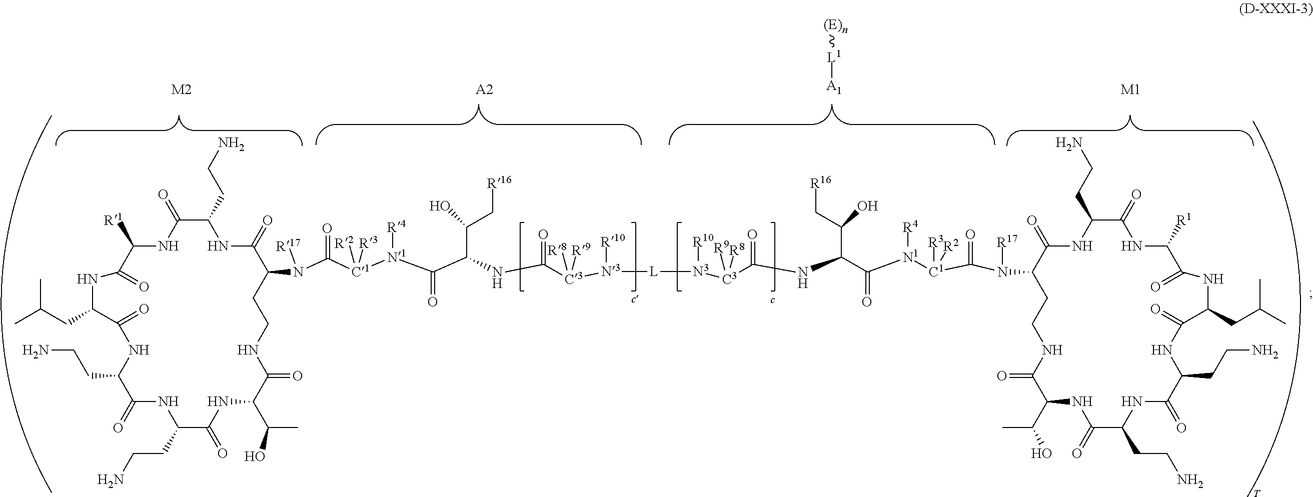

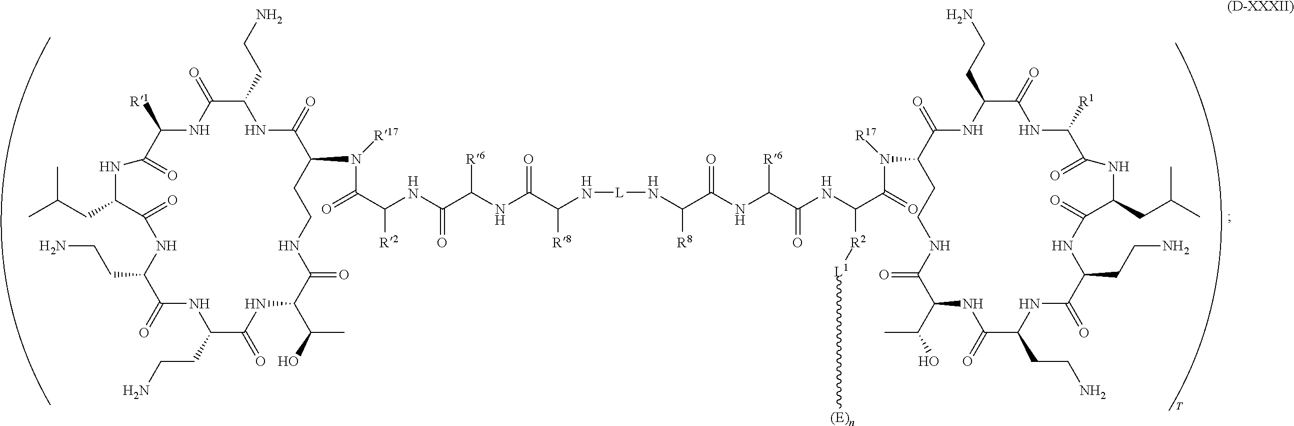

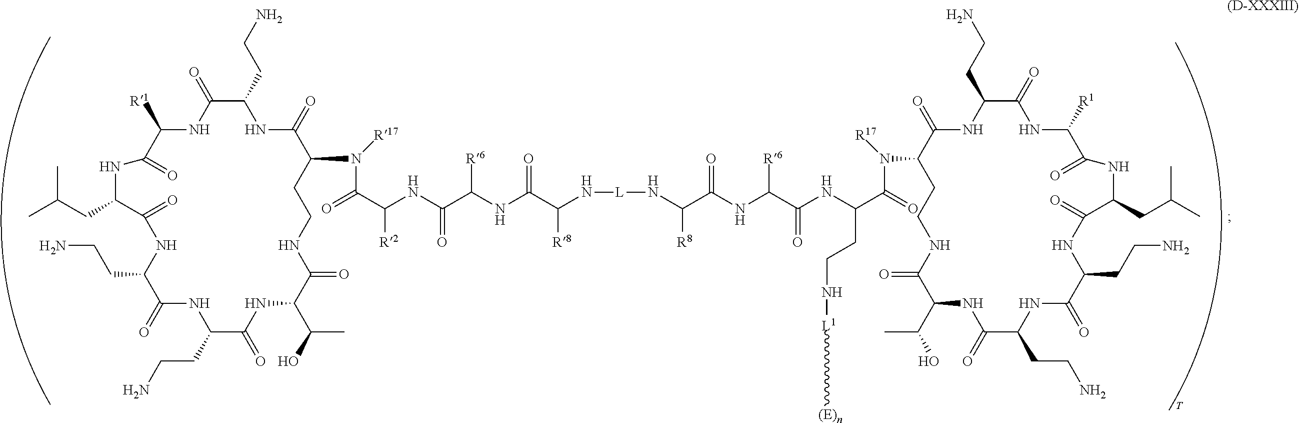

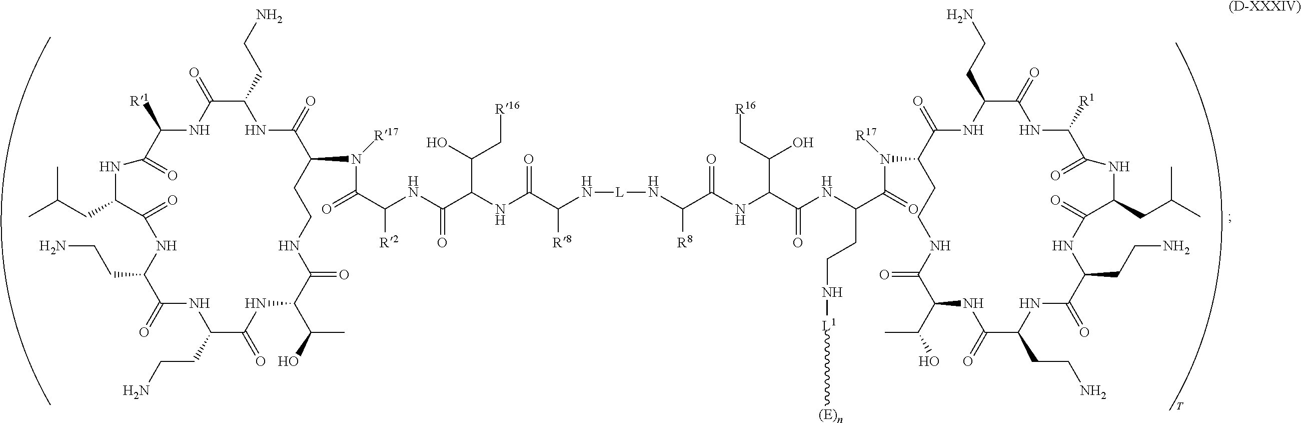

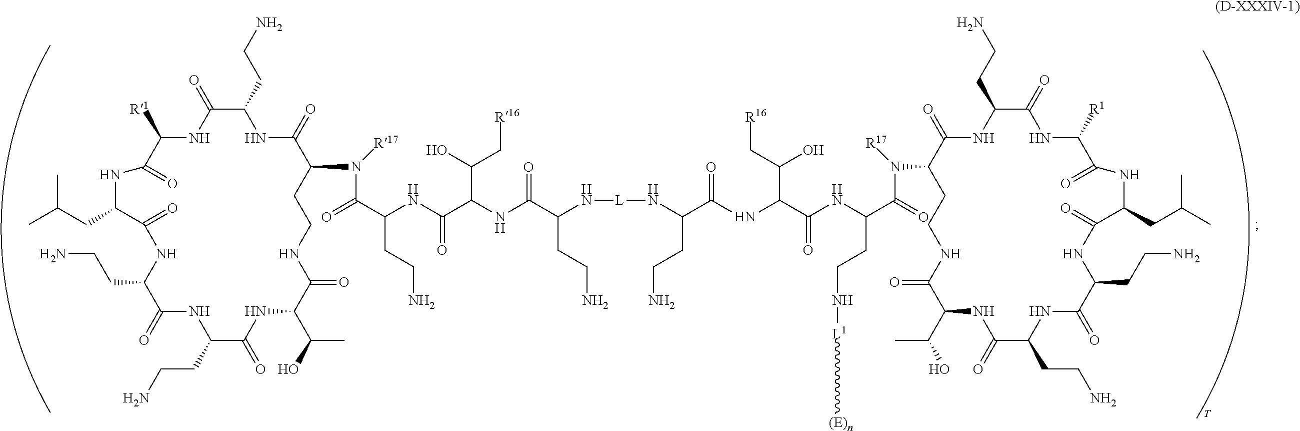

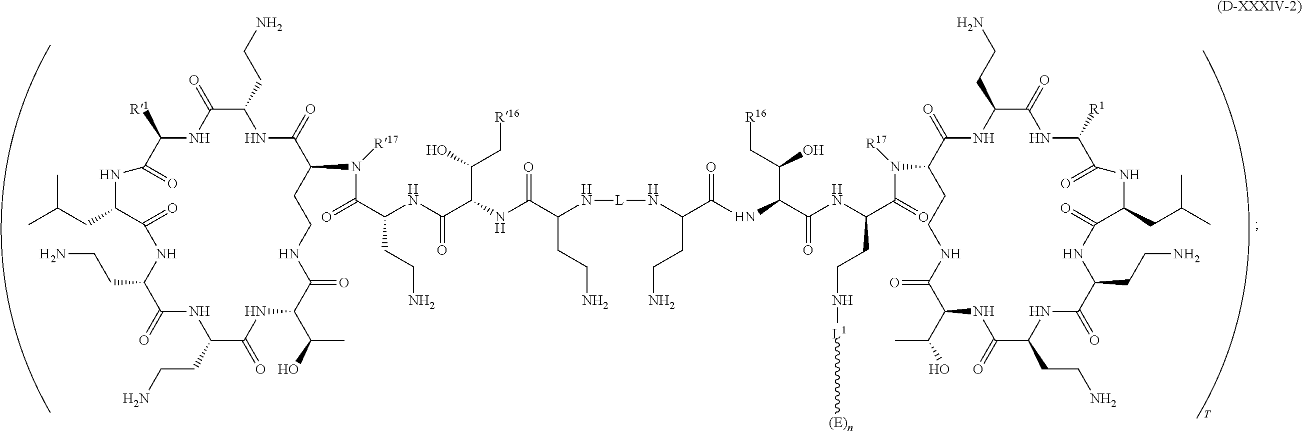

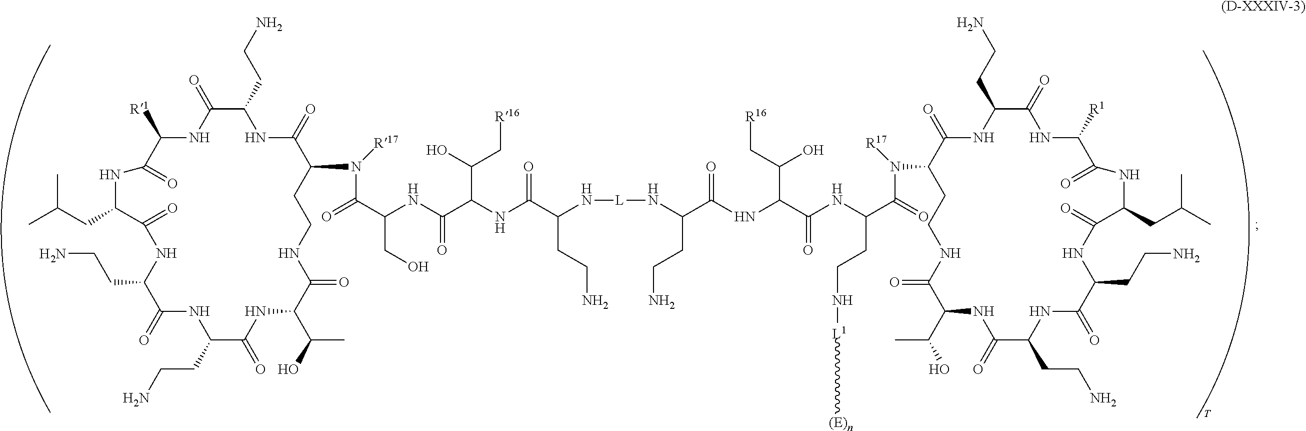

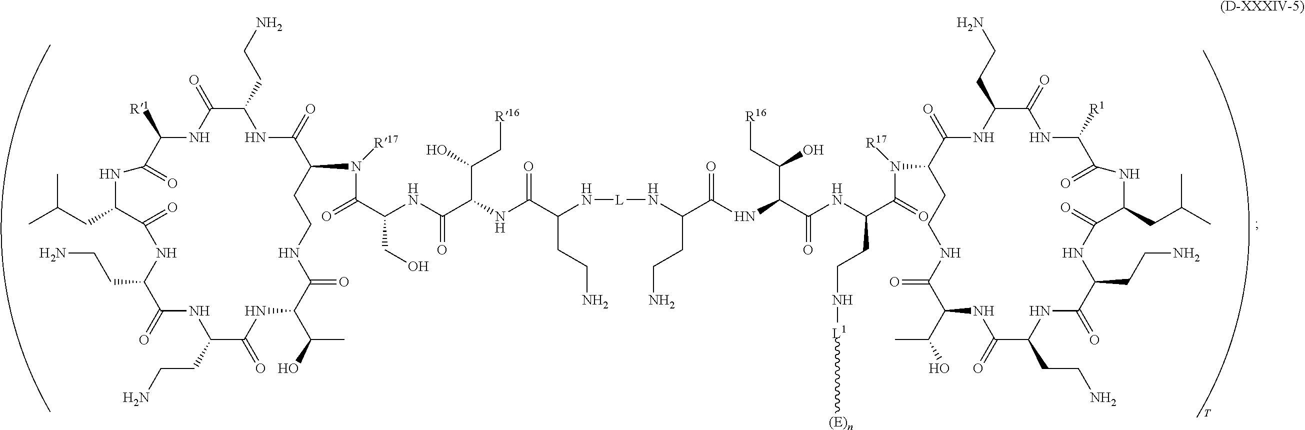

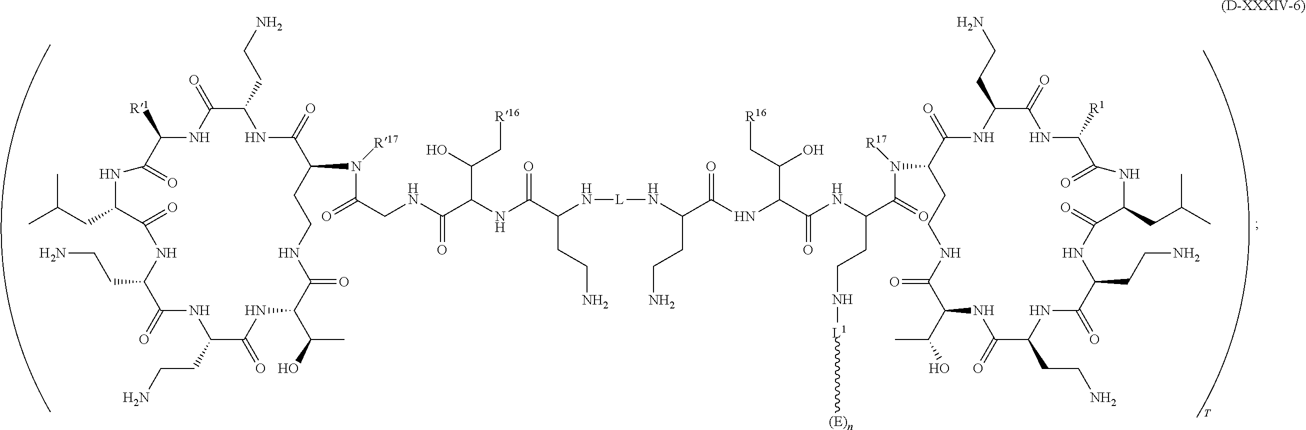

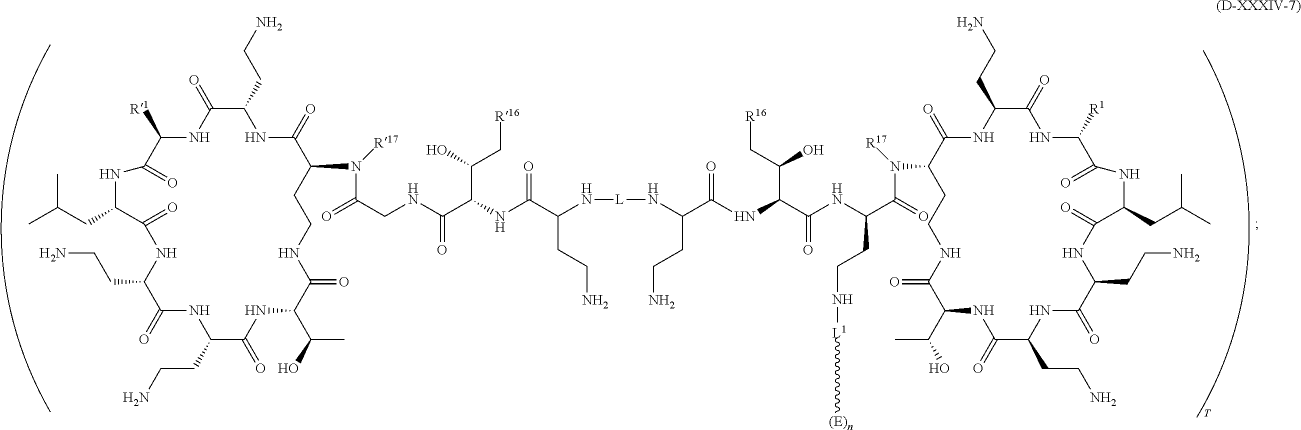

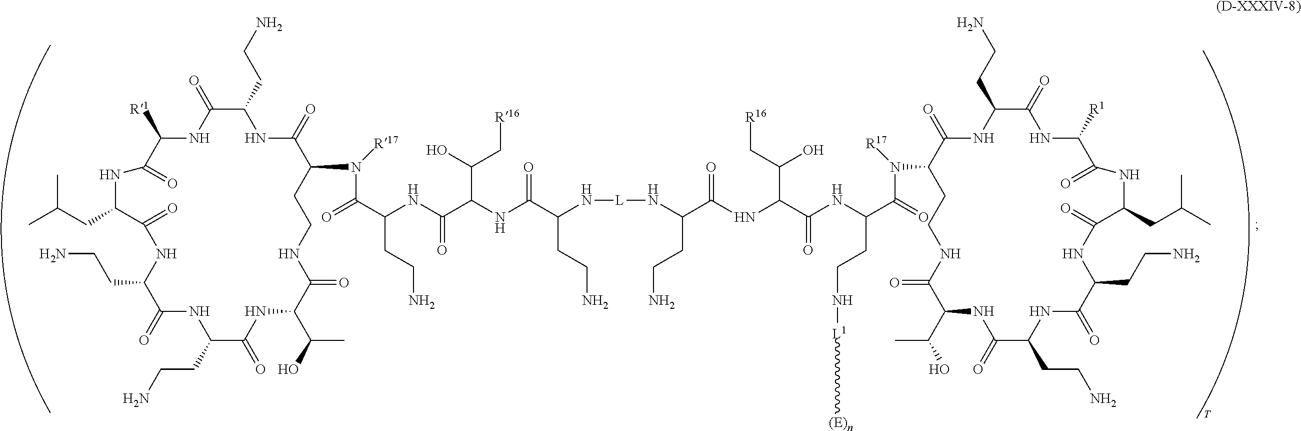

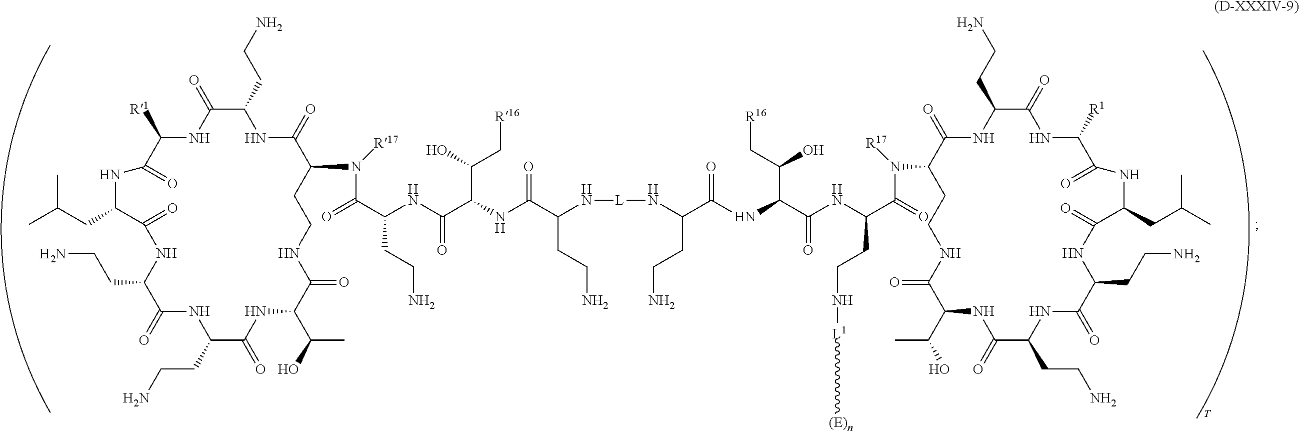

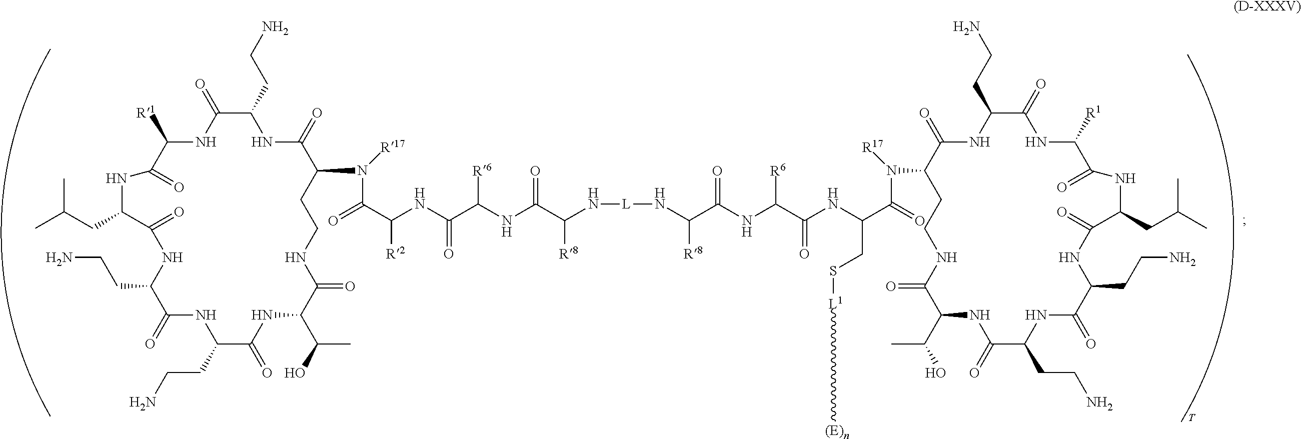

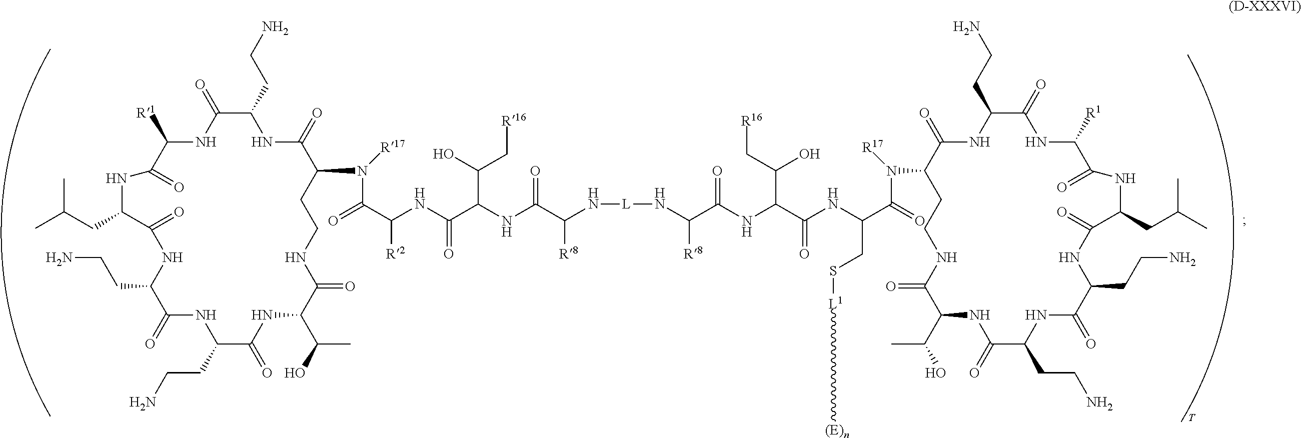

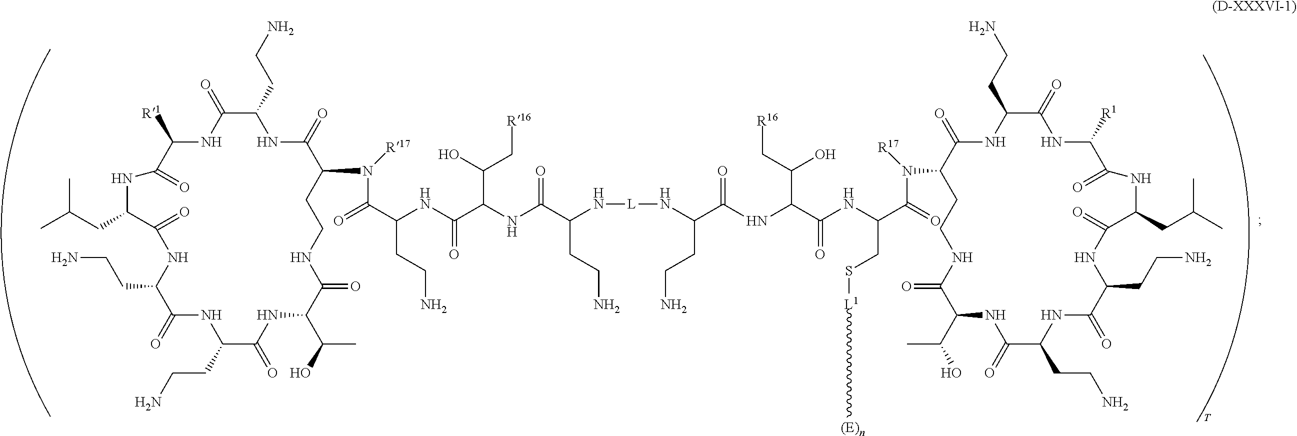

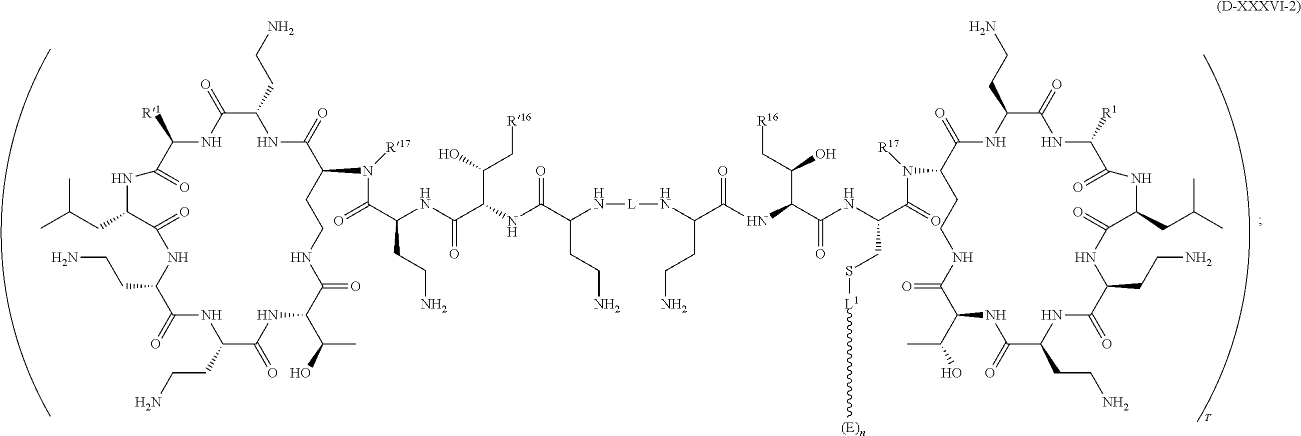

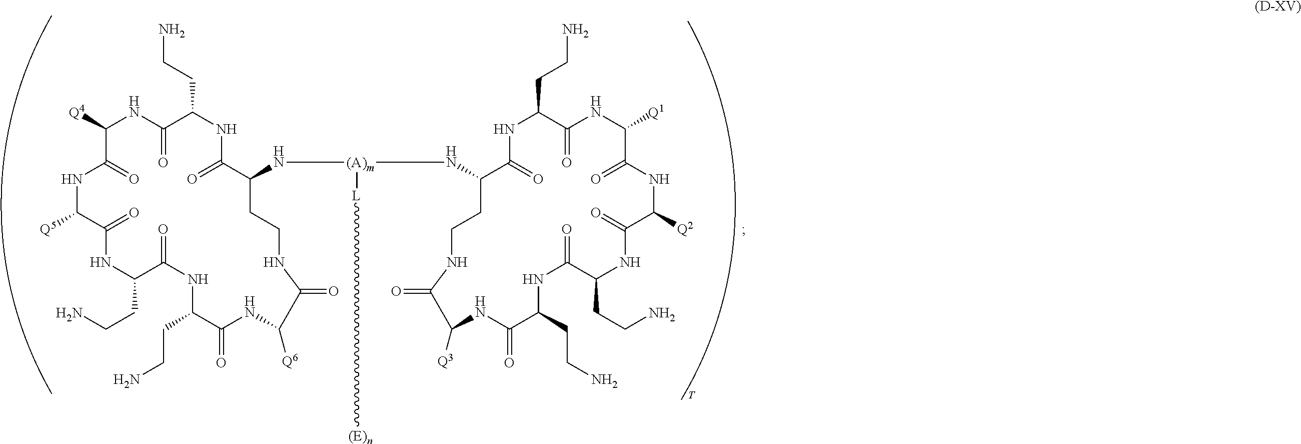

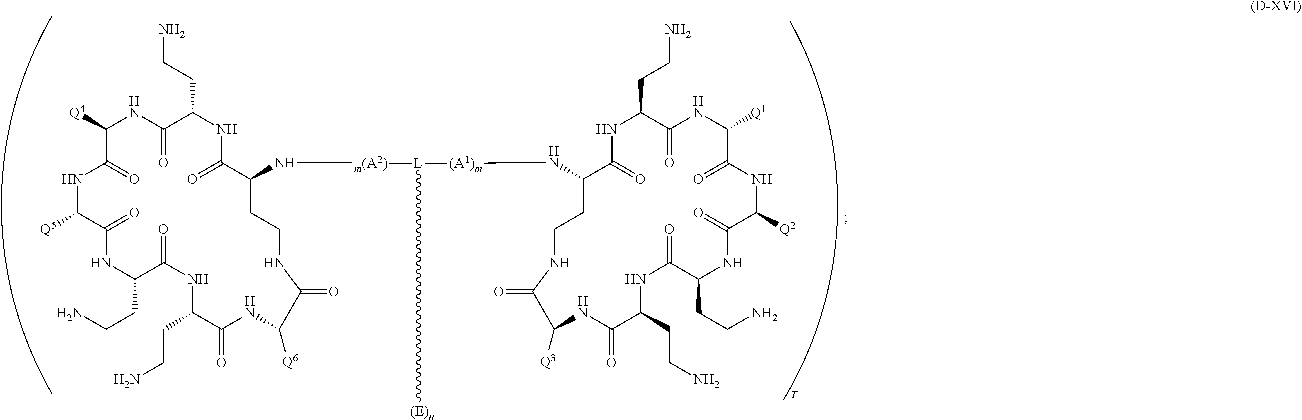

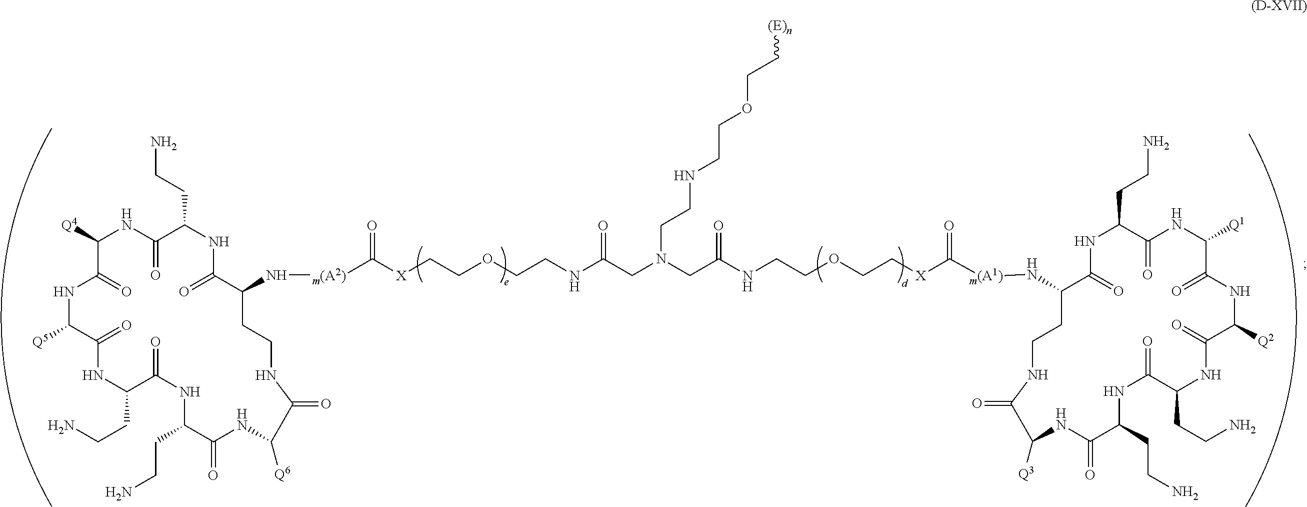

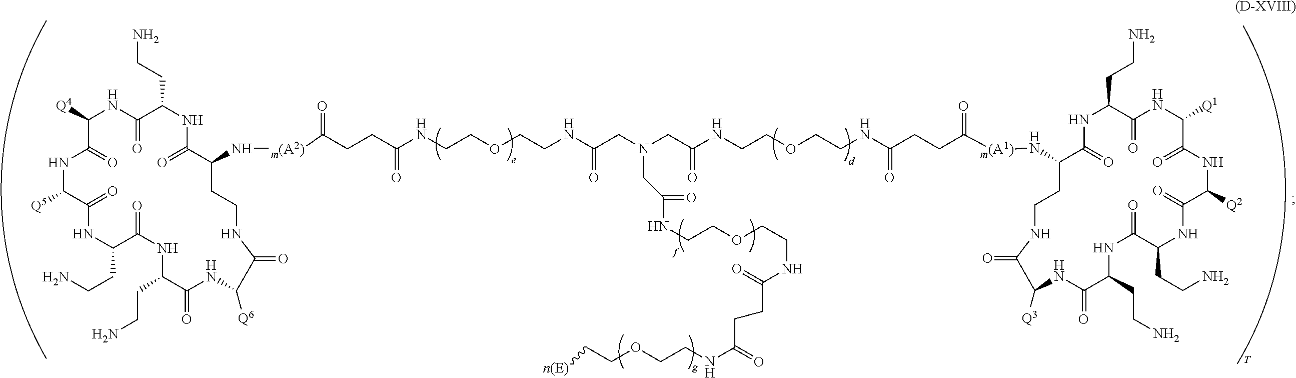

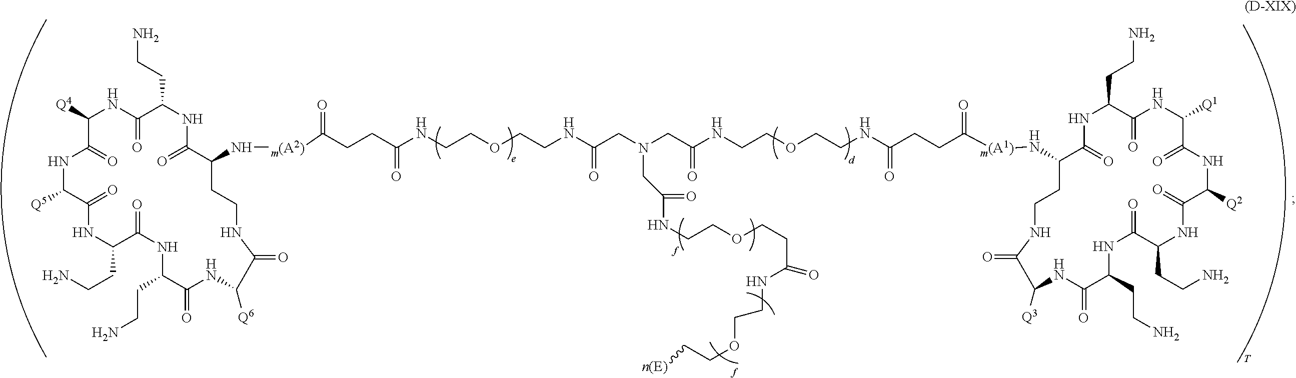

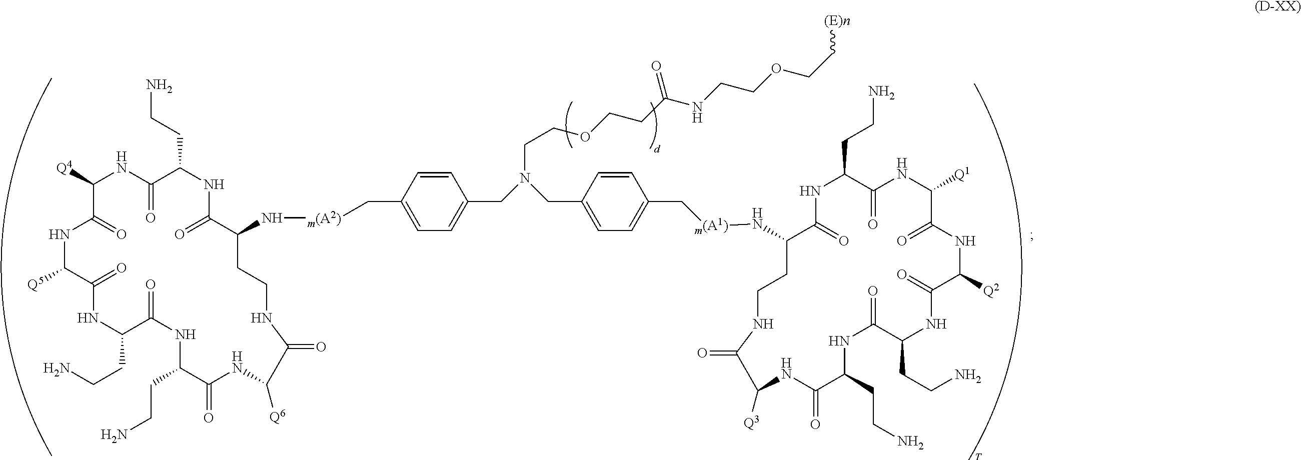

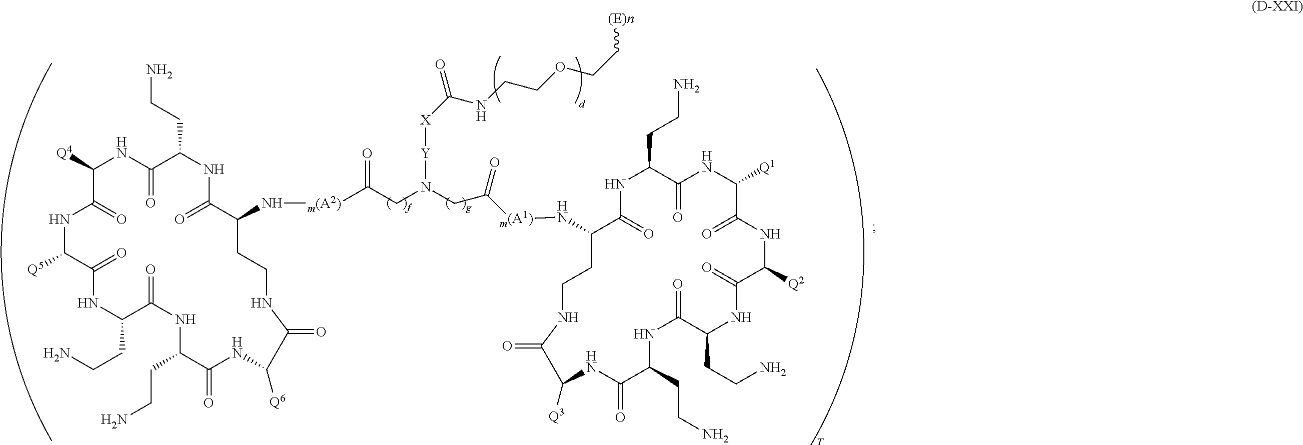

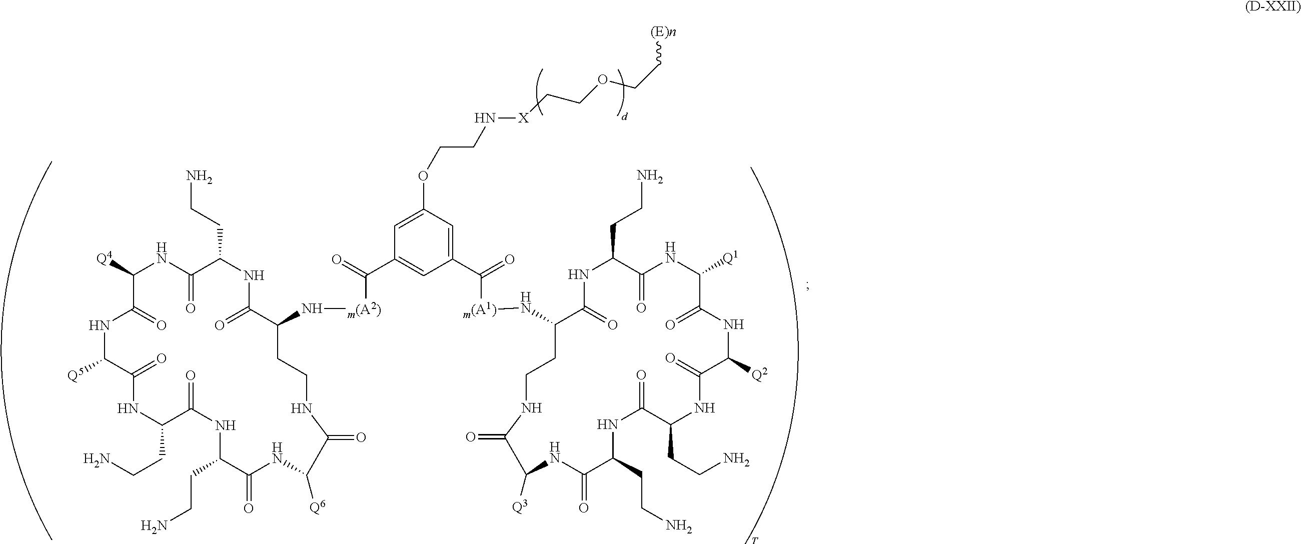

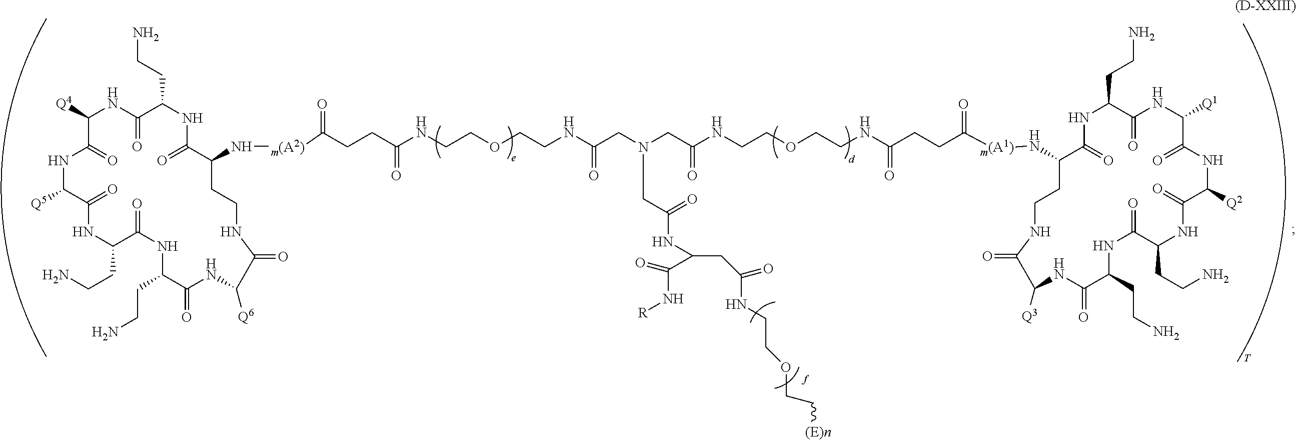

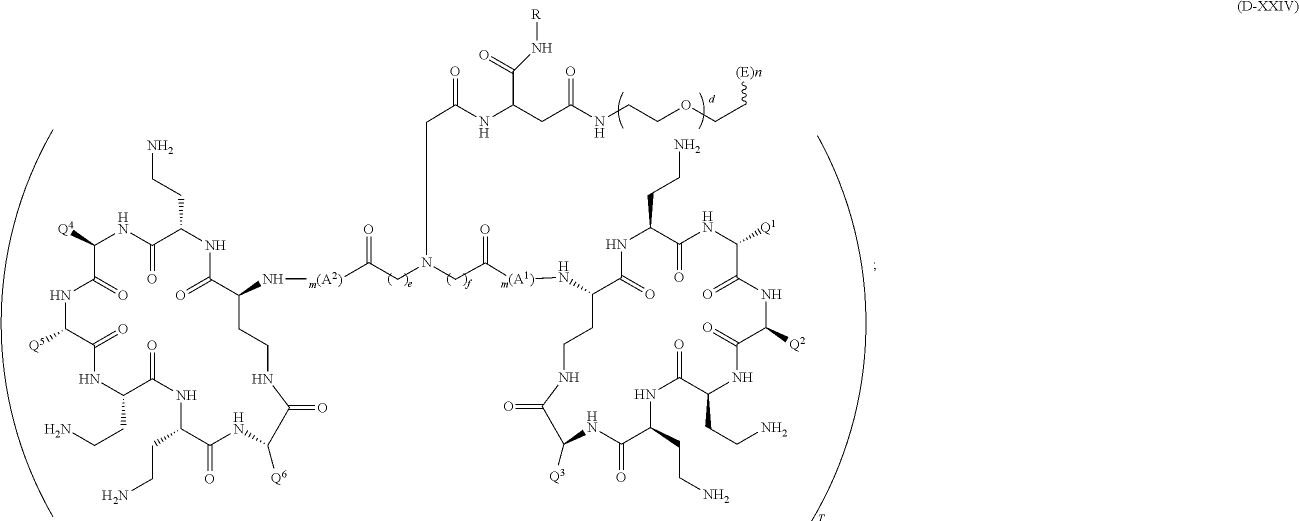

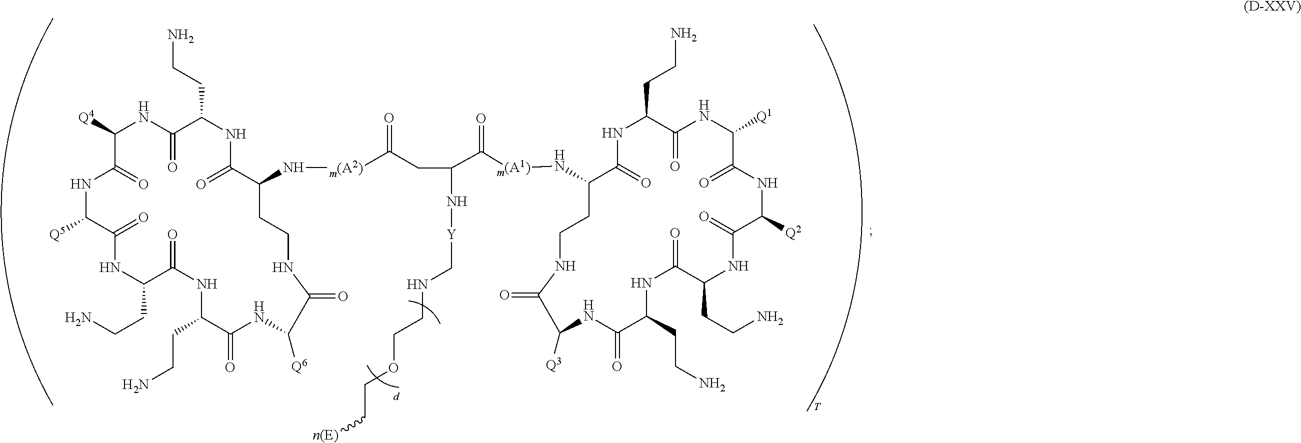

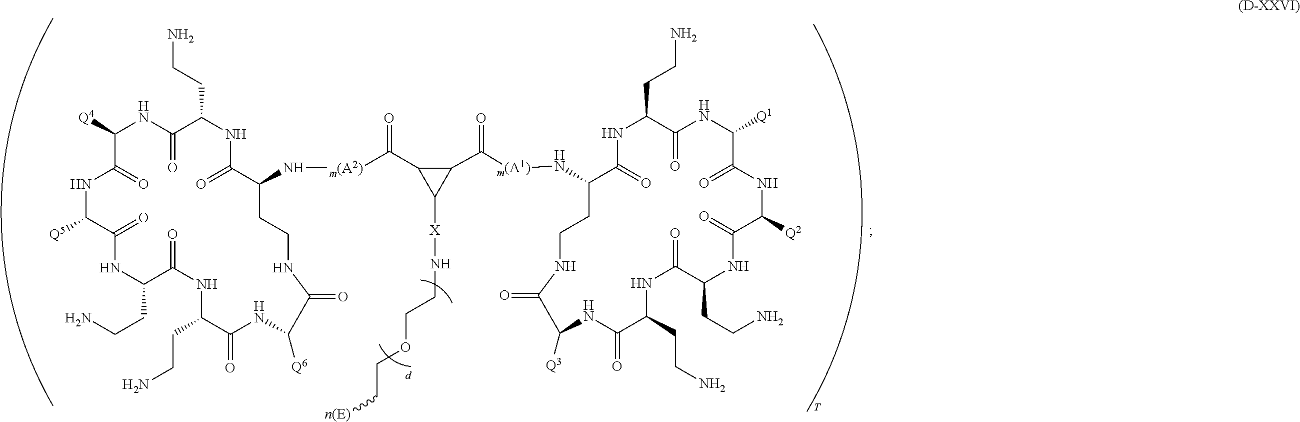

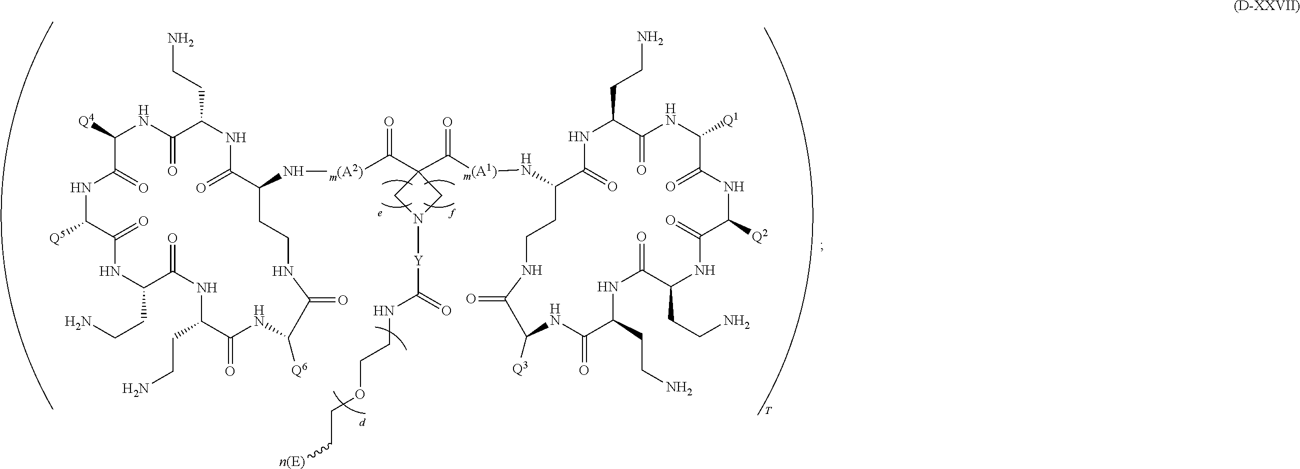

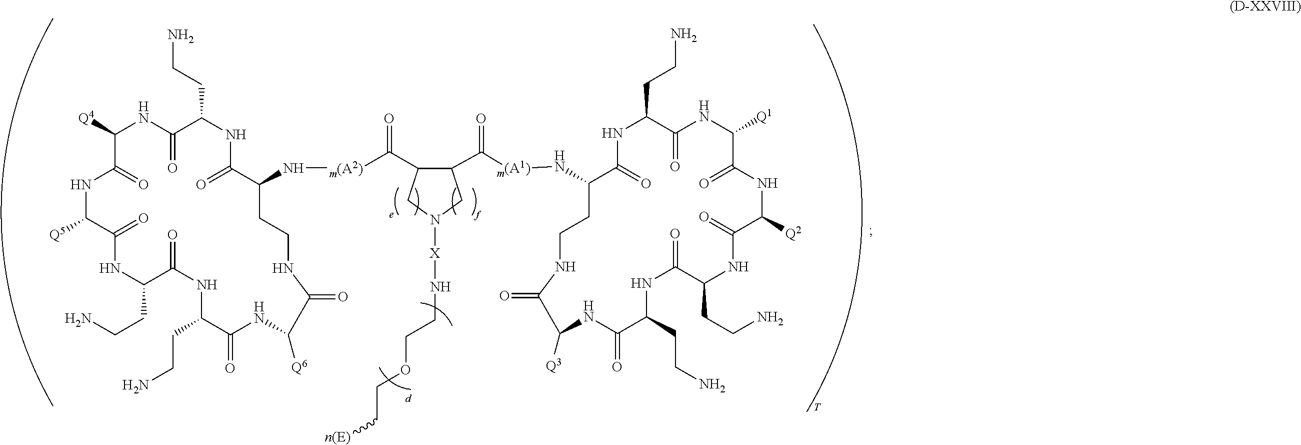

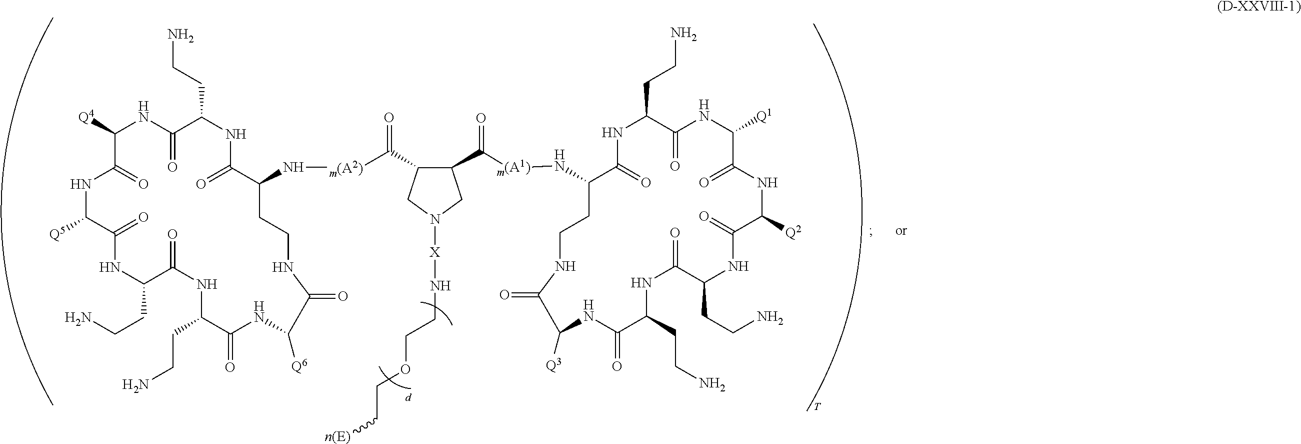

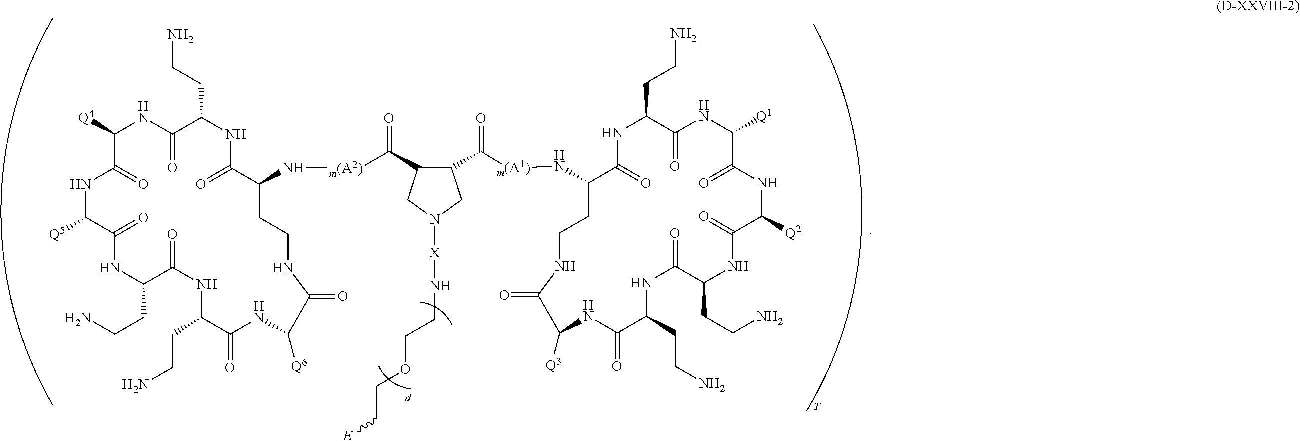

In some embodiments, when a is 1 in the conjugate of formula (II), (1) each of R², R³, and R⁴ is, independently, a lipophilic moiety, a positively charged moiety, a polar moiety, H, optionally substituted alkamino, optionally substituted alkyl, optionally substituted cycloalkyl, optionally substituted cycloalkenylene, optionally substituted cycloalkynylene, optionally substituted aryl, optionally substituted heteroalkyl, optionally substituted heterocycloalkyl, optionally substituted heterocycloalkenylene, optionally substituted heterocycloalkynylene, optionally substituted heteroaryl, optionally substituted alkaryl, or optionally substituted heteroalkaryl; or (ii) R², R³, and C¹ together form a ring (e.g., an optionally substituted 3-8 or 5-8 membered ring) comprising optionally substituted cycloalkyl, optionally substituted heterocycloalkyl comprising 1 or 2 heteroatoms independently selected from N, O, and S, and R⁴ is a lipophilic moiety, a positively charged moiety, a polar moiety, H, optionally substituted alkamino, optionally substituted alkyl, optionally substituted cycloalkyl, optionally substituted cycloalkenylene, optionally substituted cycloalkynylene, optionally substituted aryl, optionally substituted heteroalkyl, optionally substituted heterocycloalkyl, optionally substituted heterocycloalkenylene, optionally substituted heterocycloalkynylene, optionally substituted heteroaryl, optionally substituted alkaryl, or optionally substituted heteroalkaryl; or (iii) R³, R⁴, N¹, and C¹ together form a ring (e.g., an optionally substituted 3-8 or 5-8 membered ring) comprising optionally substituted heterocycloalkyl comprising an N heteroatom and additional 0-2 heteroatoms independently selected from N, O, and S, or optionally substituted heteroaryl comprising an N heteroatom and additional 0-2 heteroatoms independently selected from N, O, and S, and R² is a lipophilic moiety, a positively charged moiety, a polar moiety, H, optionally substituted alkamino, optionally substituted alkyl, optionally substituted cycloalkyl, optionally substituted cycloalkenylene, optionally substituted cycloalkynylene, optionally substituted aryl, optionally substituted heteroalkyl, optionally substituted heterocycloalkyl, optionally substituted heterocycloalkenylene, optionally substituted heterocycloalkynylene, optionally substituted heteroaryl, optionally substituted alkaryl, or optionally substituted heteroalkaryl.

In some embodiments, when b is 1 in the conjugate of formula (II), (i) each of R⁵, R⁶, and R⁷ is, independently, a lipophilic moiety, a positively charged moiety, a polar moiety, H, optionally substituted alkamino, optionally substituted alkyl, optionally substituted cycloalkyl, optionally substituted cycloalkenylene, optionally substituted cycloalkynylene, optionally substituted aryl, optionally substituted heteroalkyl, optionally substituted heterocycloalkyl, optionally substituted heterocycloalkenylene, optionally substituted heterocycloalkynylene, optionally substituted heteroaryl, optionally substituted alkaryl, or optionally substituted heteroalkaryl; or (ii) R⁵, R⁶, and C² together form a ring (e.g., an optionally substituted 3-8 or 5-8 membered ring) comprising optionally substituted cycloalkyl, optionally substituted heterocycloalkyl comprising 1 or 2 heteroatoms independently selected from N, O, and S, and R⁷ is a lipophilic moiety, a positively charged moiety, a polar moiety. H, optionally substituted alkamino, optionally substituted alkyl, optionally substituted cycloalkyl, optionally substituted cycloalkenylene, optionally substituted cycloalkynylene, optionally substituted aryl, optionally substituted heteroalkyl, optionally substituted heterocycloalkyl, optionally substituted heterocycloalkenylene, optionally substituted heterocycloalkynylene, optionally substituted heteroaryl, optionally substituted alkaryl, or optionally substituted heteroalkaryl; or (iii) R⁶, R⁷, N², and C² together form a ring (e.g., an optionally substituted 5-8 membered ring) comprising optionally substituted heterocycloalkyl comprising an N heteroatom and additional 0-2 heteroatoms independently selected from N, O, and S, or optionally substituted heteroaryl comprising an N heteroatom and additional 0-2 heteroatoms independently selected from N, O, and S, and R⁵ is a lipophilic moiety, a positively charged moiety, a polar moiety, H, optionally substituted alkamino, optionally substituted alkyl, optionally substituted cycloalkyl, optionally substituted cycloalkenylene, optionally substituted cycloalkynylene, optionally substituted aryl, optionally substituted heteroalkyl, optionally substituted heterocycloalkyl, optionally substituted heterocycloalkenylene, optionally substituted heterocycloalkynylene, optionally substituted heteroaryl, optionally substituted alkaryl, or optionally substituted heteroalkaryl.

In some embodiments, when c is 1 in the conjugate of formula (II), (i) each of R⁸, R⁹, and R¹⁰ is, independently, a lipophilic moiety, a positively charged moiety, a polar moiety, H, optionally substituted alkamino, optionally substituted alkyl, optionally substituted cycloalkyl, optionally substituted cycloalkenylene, optionally substituted cycloalkynylene, optionally substituted aryl, optionally substituted heteroalkyl, optionally substituted heterocycloalkyl, optionally substituted heterocycloalkenylene, optionally substituted heterocycloalkynylene, optionally substituted heteroaryl, optionally substituted alkaryl, or optionally substituted heteroalkaryl; or (ii) R⁸, R⁹, and C³ together form a ring (e.g., an optionally substituted 5-8 membered ring) comprising optionally substituted cycloalkyl, optionally substituted heterocycloalkyl comprising 1 or 2 heteroatoms independently selected from N, O, and S, and R¹⁰ is a lipophilic moiety, a positively charged moiety, a polar moiety, H, optionally substituted alkamino, optionally substituted alkyl, optionally substituted cycloalkyl, optionally substituted cycloalkenylene, optionally substituted cycloalkynylene, optionally substituted aryl, optionally substituted heteroalkyl, optionally substituted heterocycloalkyl, optionally substituted heterocycloalkenylene, optionally substituted heterocycloalkynylene, optionally substituted heteroaryl, optionally substituted alkaryl, or optionally substituted heteroalkaryl; or (iii) R⁹, R¹⁰, N³, and C³ together form a ring (e.g., an optionally substituted 5-8 membered ring) comprising optionally substituted heterocycloalkyl comprising an N heteroatom and additional 0-2 heteroatoms independently selected from N, O, and S, or optionally substituted heteroaryl comprising an N heteroatom and additional 0-2 heteroatoms independently selected from N, O, and S, and R⁸ is a lipophilic moiety, a positively charged moiety, a polar moiety, H, optionally substituted alkamino, optionally substituted alkyl, optionally substituted cycloalkyl, optionally substituted cycloalkenylene, optionally substituted cycloalkynylene, optionally substituted aryl, optionally substituted heteroalkyl, optionally substituted heterocycloalkyl, optionally substituted heterocycloalkenylene, optionally substituted heterocycloalkynylene, optionally substituted heteroaryl, optionally substituted alkaryl, or optionally substituted heteroalkaryl.

In some embodiments, when a′ is 1 in the conjugate of formula (II), (i) each of R′², R′³, and R′⁴ is, independently, a lipophilic moiety, a positively charged moiety, a polar moiety. H, optionally substituted alkamino, optionally substituted alkyl, optionally substituted cycloalkyl, optionally substituted cycloalkenylene, optionally substituted cycloalkynylene, optionally substituted aryl, optionally substituted heteroalkyl, optionally substituted heterocycloalkyl, optionally substituted heterocycloalkenylene, optionally substituted heterocycloalkynylene, optionally substituted heteroaryl, optionally substituted alkaryl, or optionally substituted heteroalkaryl; or (ii) R′², R′³, and C′¹ together form a ring (e.g., an optionally substituted 5-8 membered ring) comprising optionally substituted cycloalkyl, optionally substituted heterocycloalkyl comprising 1 or 2 heteroatoms independently selected from N, O, and S, and R′4 is a lipophilic moiety, a positively charged moiety, a polar moiety, H, optionally substituted alkamino, optionally substituted alkyl, optionally substituted cycloalkyl, optionally substituted cycloalkenylene, optionally substituted cycloalkynylene, optionally substituted aryl, optionally substituted heteroalkyl, optionally substituted heterocycloalkyl, optionally substituted heterocycloalkenylene, optionally substituted heterocycloalkynylene, optionally substituted heteroaryl, optionally substituted alkaryl, or optionally substituted heteroalkaryl; or (iii) R′³, R′⁴, N′¹, and C′¹ together form a ring (e.g., an optionally substituted 5-8 membered ring) comprising optionally substituted heterocycloalkyl comprising an N heteroatom and additional 0-2 heteroatoms independently selected from N, O, and S, or optionally substituted heteroaryl comprising an N heteroatom and additional 0-2 heteroatoms independently selected from N, O, and S, and R′² is a lipophilic moiety, a positively charged moiety, a polar moiety, H, optionally substituted alkamino, optionally substituted alkyl, optionally substituted cycloalkyl, optionally substituted cycloalkenylene, optionally substituted cycloalkynylene, optionally substituted aryl, optionally substituted heteroalkyl, optionally substituted heterocycloalkyl, optionally substituted heterocycloalkenylene, optionally substituted heterocycloalkynylene, optionally substituted heteroaryl, optionally substituted alkaryl, or optionally substituted heteroalkaryl.

In some embodiments, when b′ is 1 in the conjugate of formula (II), (i) each of R′⁵, R′⁶, and R′⁷ is, independently, a lipophilic moiety, a positively charged moiety, a polar moiety, H, optionally substituted alkamino, optionally substituted alkyl, optionally substituted cycloalkyl, optionally substituted cycloalkenylene, optionally substituted cycloalkynylene, optionally substituted aryl, optionally substituted heteroalkyl, optionally substituted heterocycloalkyl, optionally substituted heterocycloalkenylene, optionally substituted heterocycloalkynylene, optionally substituted heteroaryl, optionally substituted alkaryl, or optionally substituted heteroalkaryl; or (ii) R′⁵, R′⁶, and C′² together form a ring (e.g., an optionally substituted 3-8 or 5-8 membered ring) comprising optionally substituted cycloalkyl, optionally substituted heterocycloalkyl comprising 1 or 2 heteroatoms independently selected from N, O, and S, and R′⁷ is a lipophilic moiety, a positively charged moiety, a polar moiety, H, optionally substituted alkamino, optionally substituted alkyl, optionally substituted cycloalkyl, optionally substituted cycloalkenylene, optionally substituted cycloalkynylene, optionally substituted aryl, optionally substituted heteroalkyl, optionally substituted heterocycloalkyl, optionally substituted heterocycloalkenylene, optionally substituted heterocycloalkynylene, optionally substituted heteroaryl, optionally substituted alkaryl, or optionally substituted heteroalkaryl; or (iii) R′⁶, R′⁷, N′², and C′² together form a ring (e.g., an optionally substituted 5-8 membered ring) comprising optionally substituted heterocycloalkyl comprising an N heteroatom and additional 0-2 heteroatoms independently selected from N, O, and S, or optionally substituted heteroaryl comprising an N heteroatom and additional 0-2 heteroatoms independently selected from N, O, and S, and R′⁵ is a lipophilic moiety, a positively charged moiety, a polar moiety, H, optionally substituted alkamino, optionally substituted alkyl, optionally substituted cycloalkyl, optionally substituted cycloalkenylene, optionally substituted cycloalkynylene, optionally substituted aryl, optionally substituted heteroalkyl, optionally substituted heterocycloalkyl, optionally substituted heterocycloalkenylene, optionally substituted heterocycloalkynylene, optionally substituted heteroaryl, optionally substituted alkaryl, or optionally substituted heteroalkaryl.

In some embodiments, when c′ is 1 in the conjugate of formula (II), (i) each of R′⁸, R′⁹, and R′¹⁰ is, independently, a lipophilic moiety, a positively charged moiety, a polar moiety, H, optionally substituted alkamino, optionally substituted alkyl, optionally substituted cycloalkyl, optionally substituted cycloalkenylene, optionally substituted cycloalkynylene, optionally substituted aryl, optionally substituted heteroalkyl, optionally substituted heterocycloalkyl, optionally substituted heterocycloalkenylene, optionally substituted heterocycloalkynylene, optionally substituted heteroaryl, optionally substituted alkaryl, or optionally substituted heteroalkaryl; or (ii) R′⁸, R′⁹, and C′³ together form a ring (e.g., an optionally substituted 3-8 or 5-8 membered ring) comprising optionally substituted cycloalkyl, optionally substituted heterocycloalkyl comprising 1 or 2 heteroatoms independently selected from N, O, and S, and R′¹⁰ is a lipophilic moiety, a positively charged moiety, a polar moiety, H, optionally substituted alkamino, optionally substituted alkyl, optionally substituted cycloalkyl, optionally substituted cycloalkenylene, optionally substituted cycloalkynylene, optionally substituted aryl, optionally substituted heteroalkyl, optionally substituted heterocycloalkyl, optionally substituted heterocycloalkenylene, optionally substituted heterocycloalkynylene, optionally substituted heteroaryl, optionally substituted alkaryl, or optionally substituted heteroalkaryl; or (iii) R′⁹, R′¹⁰, N′³, and C′³ together form a ring (e.g., an optionally substituted 3-8 or 5-8 membered ring) comprising optionally substituted heterocycloalkyl comprising an N heteroatom and additional 0-2 heteroatoms independently selected from N, O, and S, or optionally substituted heteroaryl comprising an N heteroatom and additional 0-2 heteroatoms independently selected from N, O, and S, and R′⁸ is a lipophilic moiety, a positively charged moiety, a polar moiety, H, optionally substituted alkamino, optionally substituted alkyl, optionally substituted cycloalkyl, optionally substituted cycloalkenylene, optionally substituted cycloalkynylene, optionally substituted aryl, optionally substituted heteroalkyl, optionally substituted heterocycloalkyl, optionally substituted heterocycloalkenylene, optionally substituted heterocycloalkynylene, optionally substituted heteroaryl, optionally substituted alkaryl, or optionally substituted heteroalkaryl.

In the conjugates described herein, the squiggly line connected to E indicates that one or more (e.g., 1, 2, 3, 4, 5, 6, 7, 8, 9, 10, 11, 12, 13, 14, 15, 16, 17, 18, 19, or 20) dimers of cyclic heptapeptides may be attached to an Fc domain monomer or an Fc domain. In some embodiments, when n is 1, one or more (e.g., 1, 2, 3, 4, 5, 6, 7, 8, 9, or 10) dimers of cyclic heptapeptides may be attached to an Fc domain monomer. In some embodiments, when n is 2, one or more (e.g., 1, 2, 3, 4, 5, 6, 7, 8, 9, 10, 11, 12, 13, 14, 15, 16, 17, 18, 19, or 20) dimers of cyclic heptapeptides may be attached to an Fc domain. The squiggly line in the conjugates described herein is not to be construed as a single bond between one or more dimers of cyclic heptapeptides and an atom in the Fc domain monomer. In some embodiments, when T is 1, one dimer of cyclic heptapeptides may be attached to an atom in the Fc domain monomer or Fc domain. In some embodiments, when T is 2, two dimers of cyclic heptapeptides may be attached to an atom in the Fc domain monomer or Fc domain.

As described further herein, a linker in a conjugate described herein (e.g., L′, L, or L¹) may be a branched structure. As described further herein, a linker in a conjugate described herein (e.g., L′, L, or L¹) may be a multivalent structure, e.g., a divalent or trivalent structure having two or three arms, respectively. In some embodiments when the linker has three arms, two of the arms may be attached to the first and second cyclic heptapeptides and the third arm may be attached to the Fc domain monomer or Fc domain.

In conjugates having an Fc domain covalently linked to one or more dimers of cyclic heptapeptides, as represented by the formulae above, when n is 2, two Fc domain monomers (each Fc domain monomer is represented by E) dimerize to form an Fc domain.

Conjugates of Monomers of Cyclic Heptapeptides Linked to an Fc Domain

In some embodiments, the conjugates described herein include an Fc domain covalently linked to one or more monomers of cyclic heptapeptides. Conjugates of an Fc domain and one or more monomers of cyclic heptapeptides may be formed by linking the Fc domain to each of the monomers of cyclic heptapeptides through a linker and/or one or more amino acids (e.g., a peptide including a 1-5 amino acid residue(s)).

In the conjugates having an Fc domain covalently linked to one or more monomers of cyclic heptapeptides described herein, the squiggly line connected to E indicates that one or more (e.g., 1, 2, 3, 4, 5, 6, 7, 8, 9, 10, 11, 12, 13, 14, 15, 16, 17, 18, 19, or 20) monomers of cyclic heptapeptides may be attached to an Fc domain monomer or an Fc domain. In some embodiments, when n is 1, one or more (e.g., 1, 2, 3, 4, 5, 6, 7, 8, 9, or 10) monomers of cyclic heptapeptides may be attached to an Fc domain monomer. In some embodiments, when n is 2, one or more (e.g., 1, 2, 3, 4, 5, 6, 7, 8, 9, 10, 11, 12, 13, 14, 15, 16, 17, 18, 19, or 20) monomers of cyclic heptapeptides may be attached to an Fc domain. The squiggly line in the conjugates described herein is not to be construed as a single bond between one or more monomers of cyclic heptapeptides and an atom in the Fc domain monomer or Fc domain. In some embodiments, when T is 1, one monomer of cyclic heptapeptide may be attached to an atom in the Fc domain monomer or Fc domain. In some embodiments, when T is 2, two monomers of cyclic heptapeptides may be attached to an atom in the Fc domain monomer or Fc domain.

As described further herein, a linker in a conjugate having an Fc domain covalently linked to one or more monomers of cyclic heptapeptides described herein (e.g., L′ or L) may be a divalent structure having two arms. One arm in a divalent linker may be attached to the monomer of cyclic heptapeptide and the arm may be attached to the Fc domain monomer or Fc domain.

In some embodiments, a conjugate containing an Fc domain covalently linked to one or more monomers of cyclic heptapeptides provided herein is described by any one of formulae below:

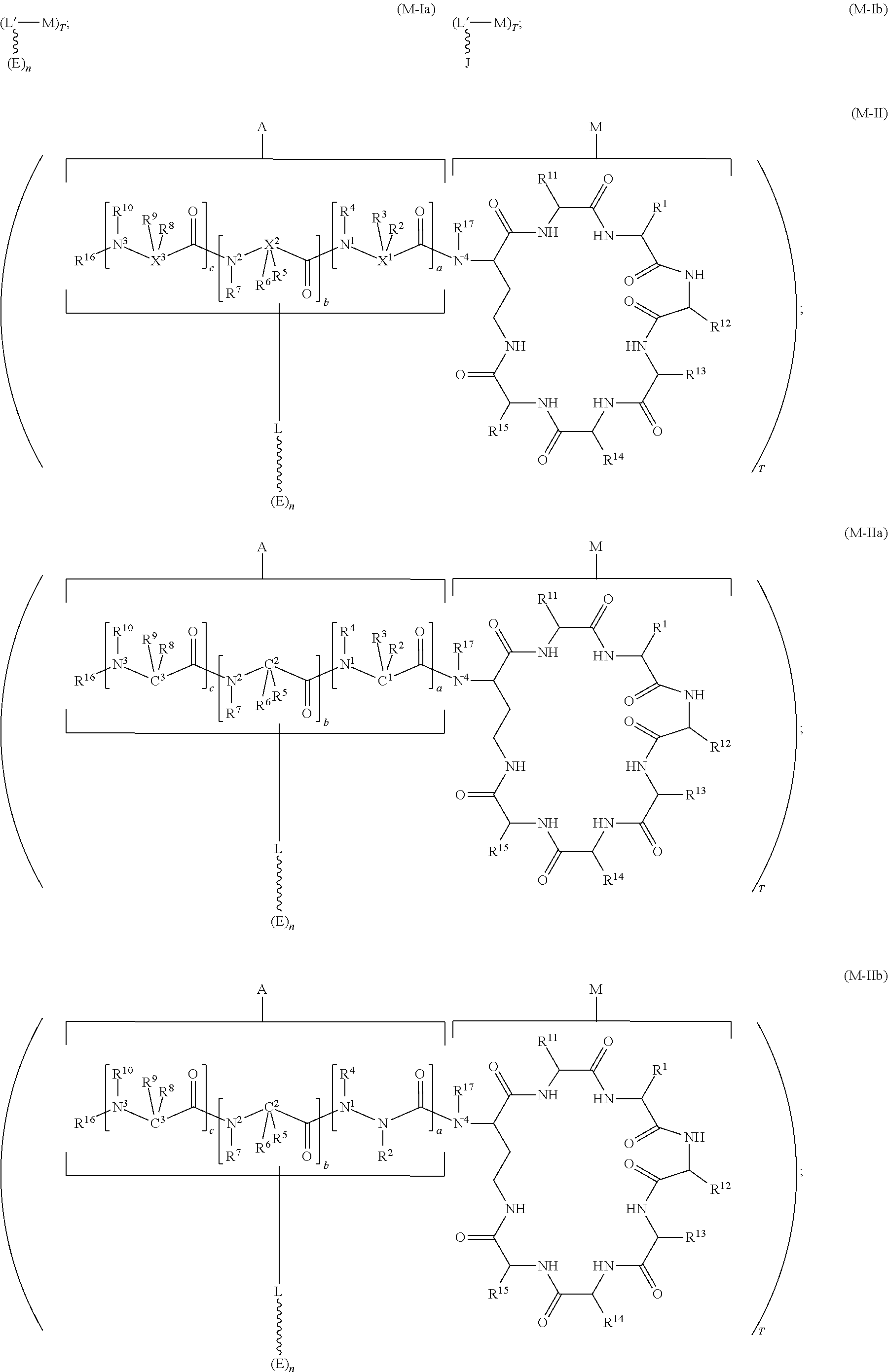

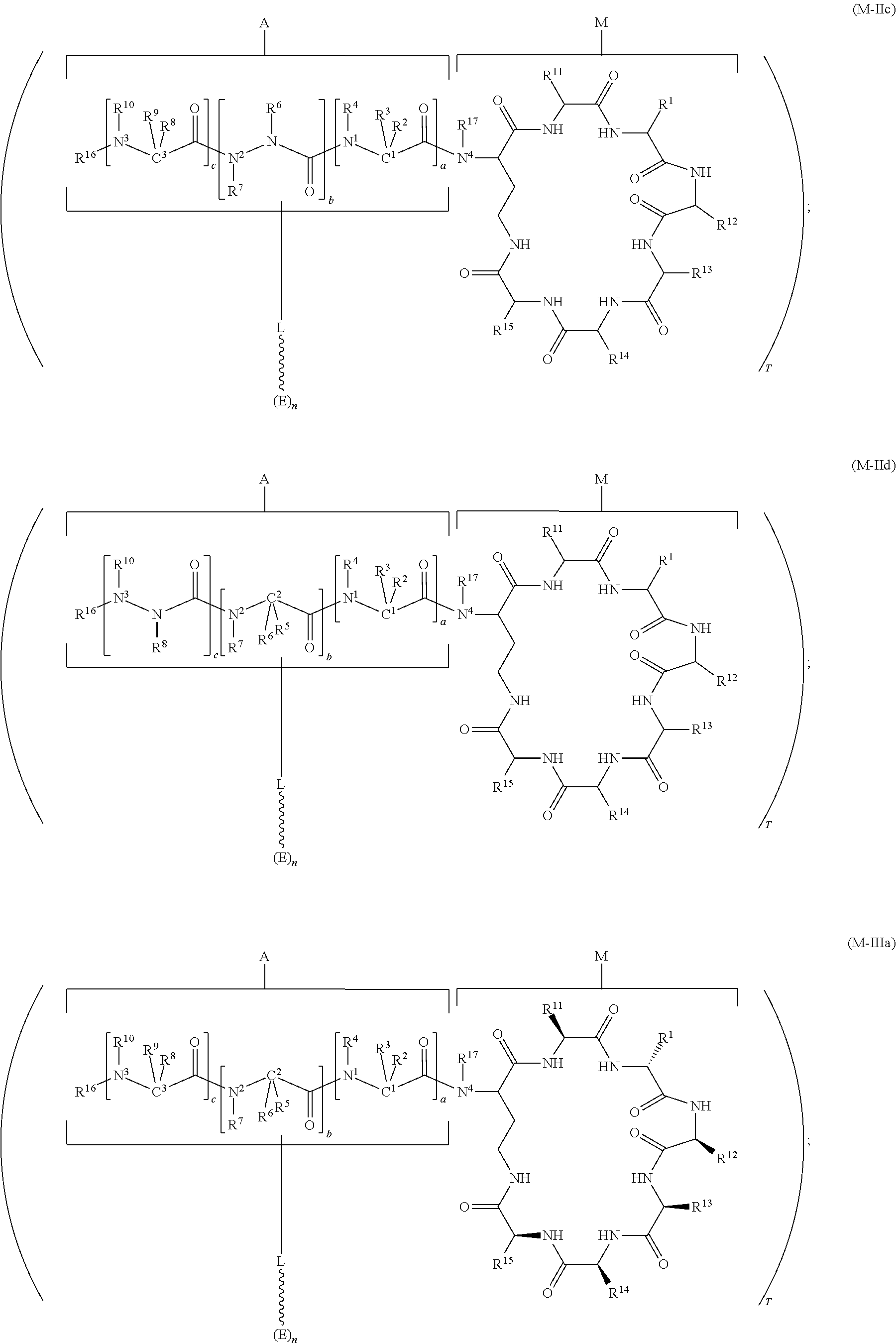

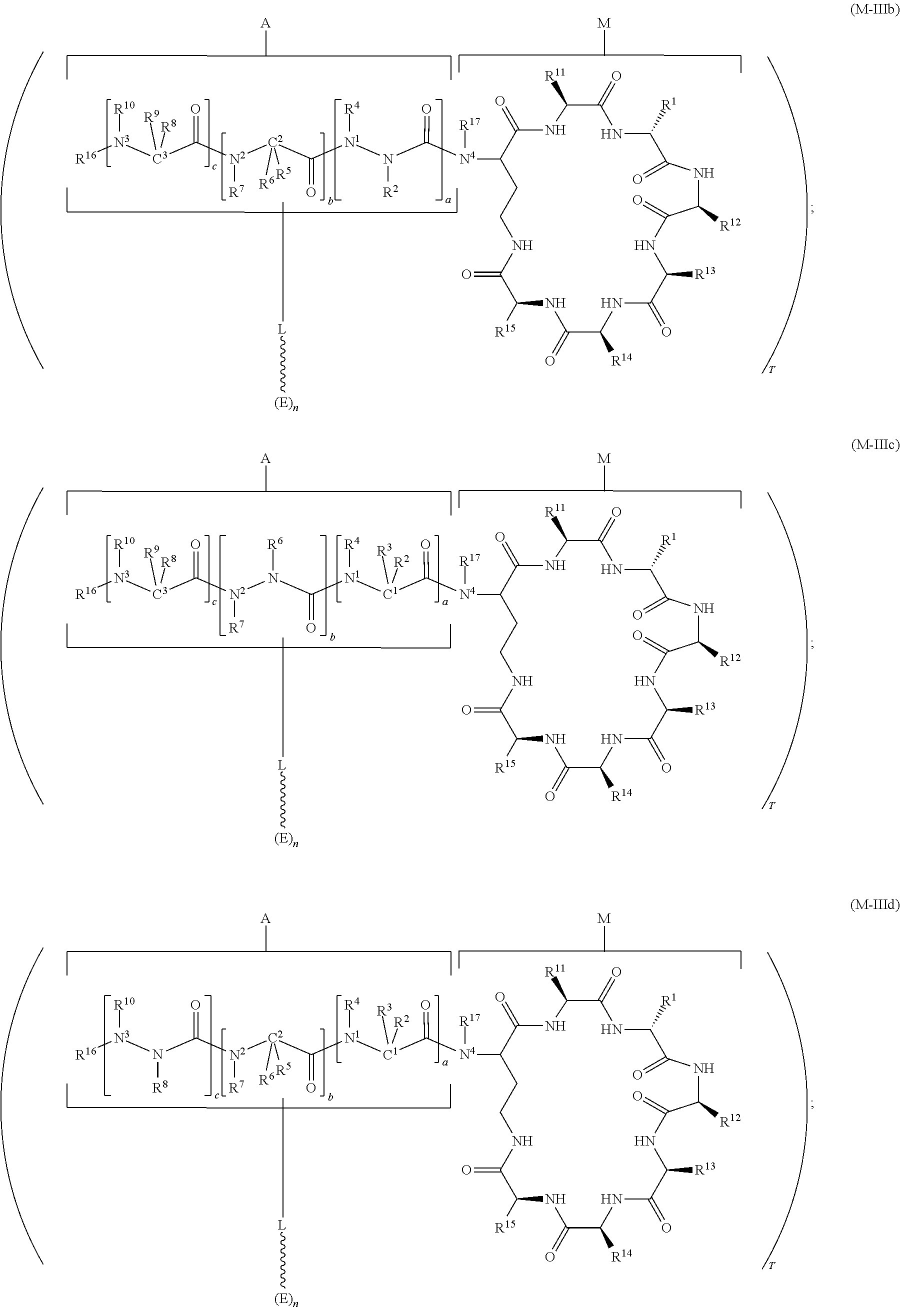

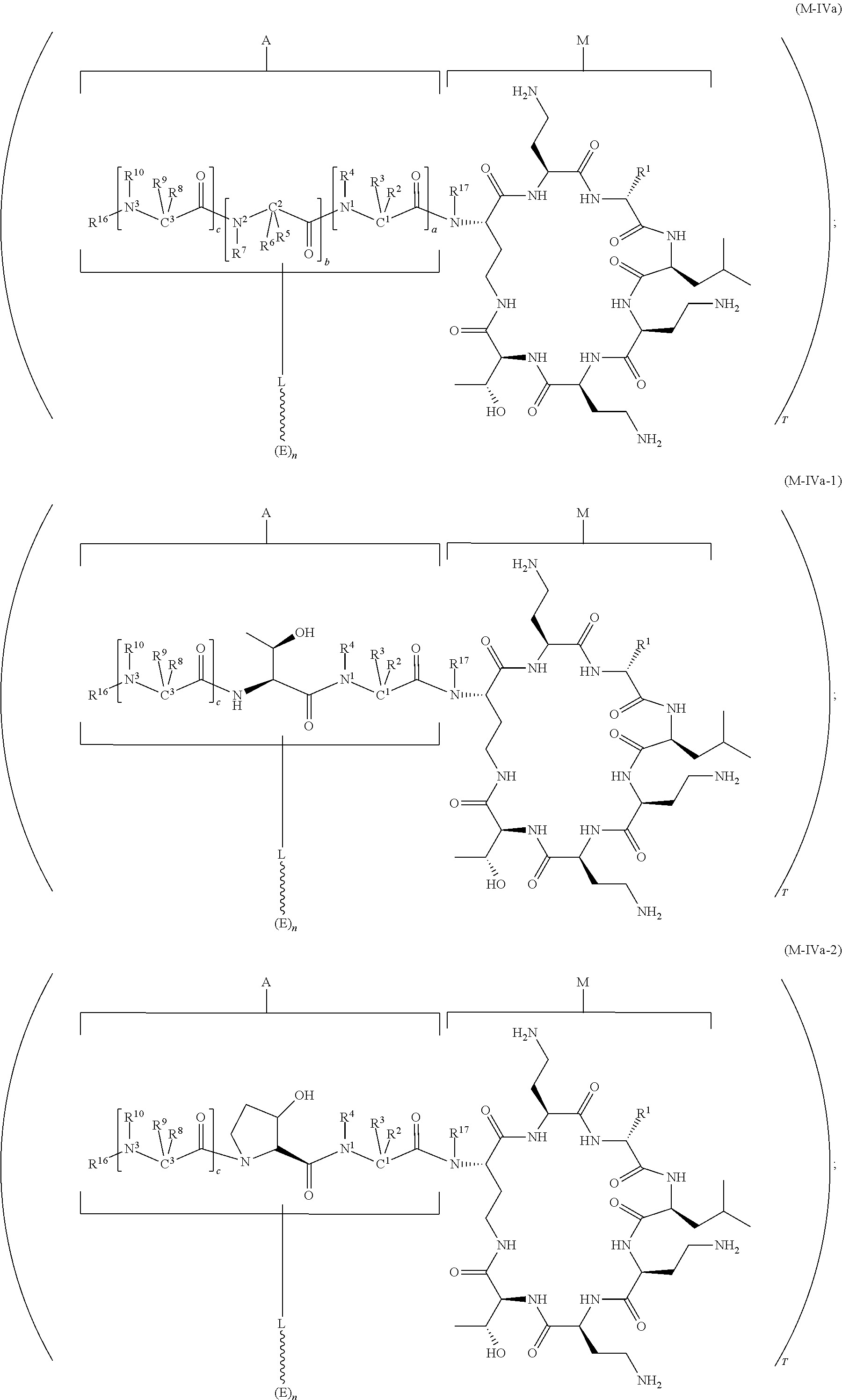

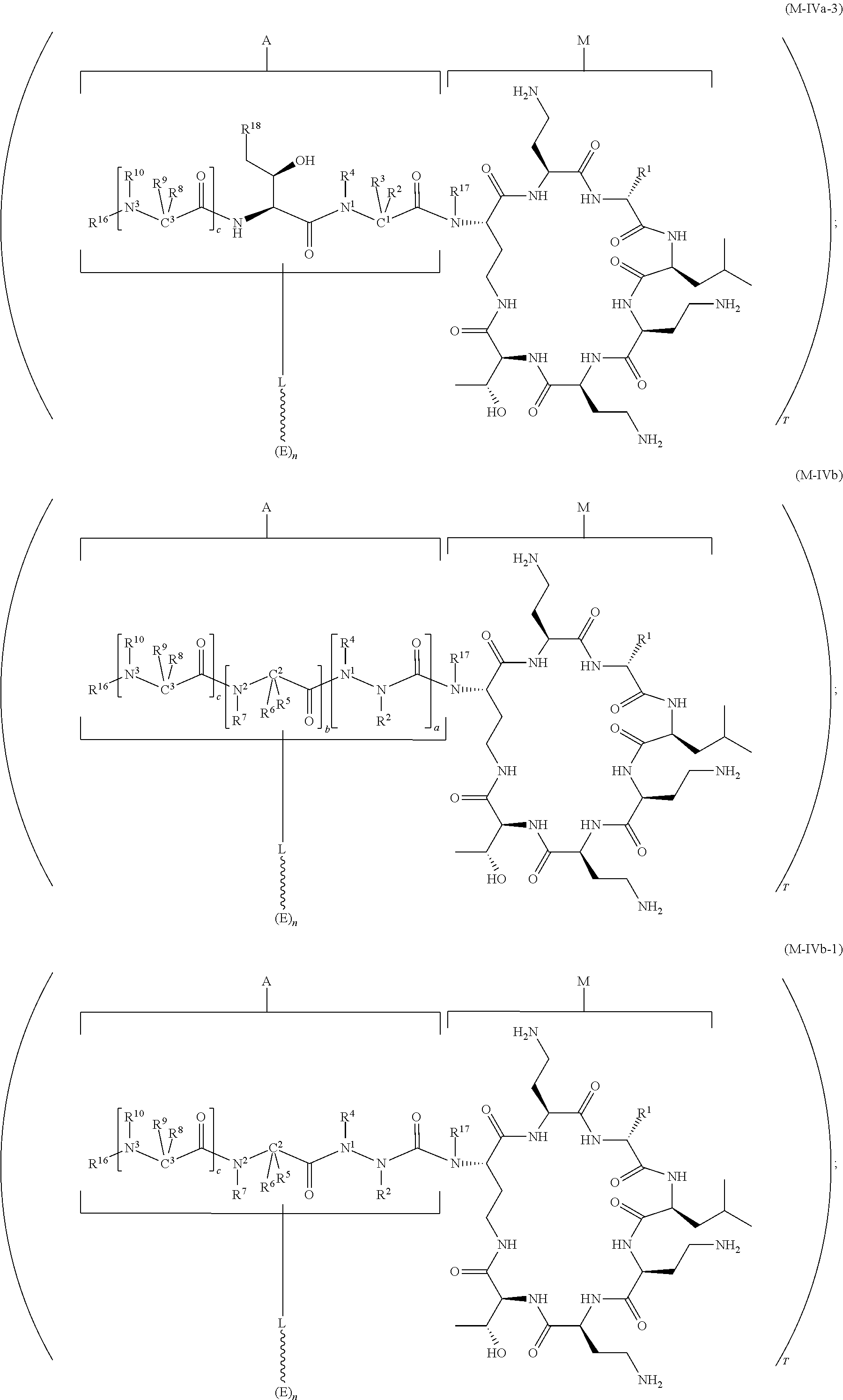

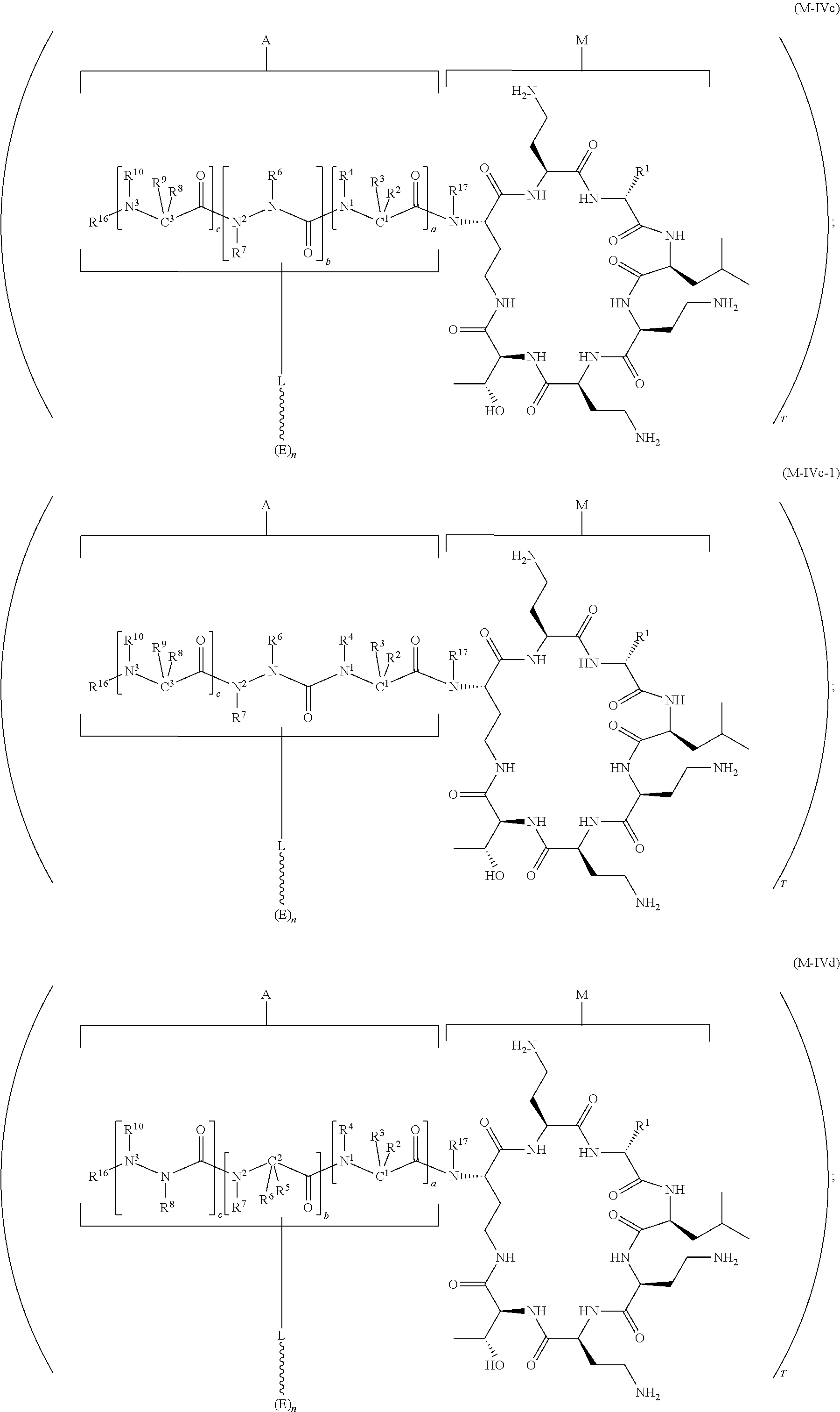

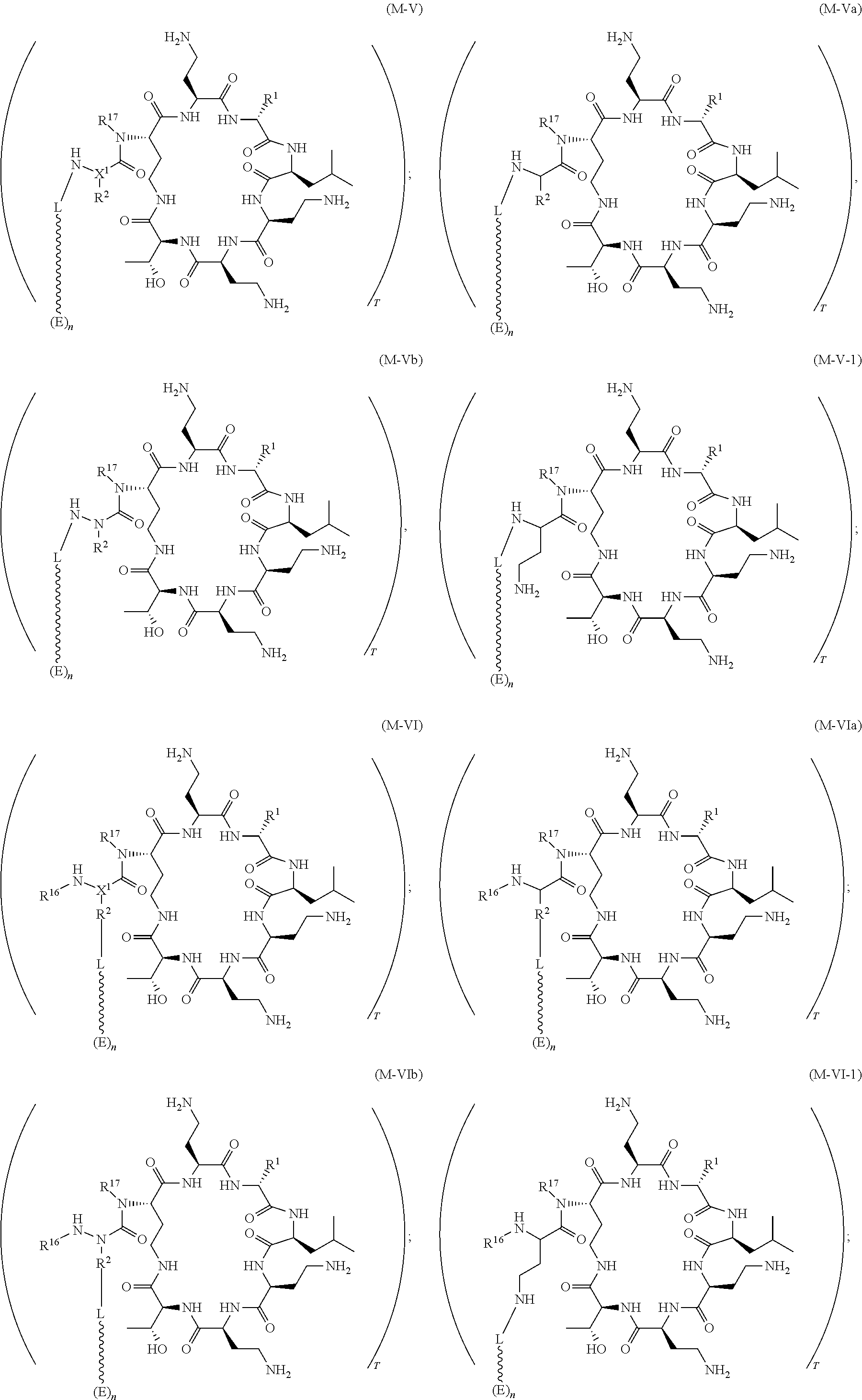

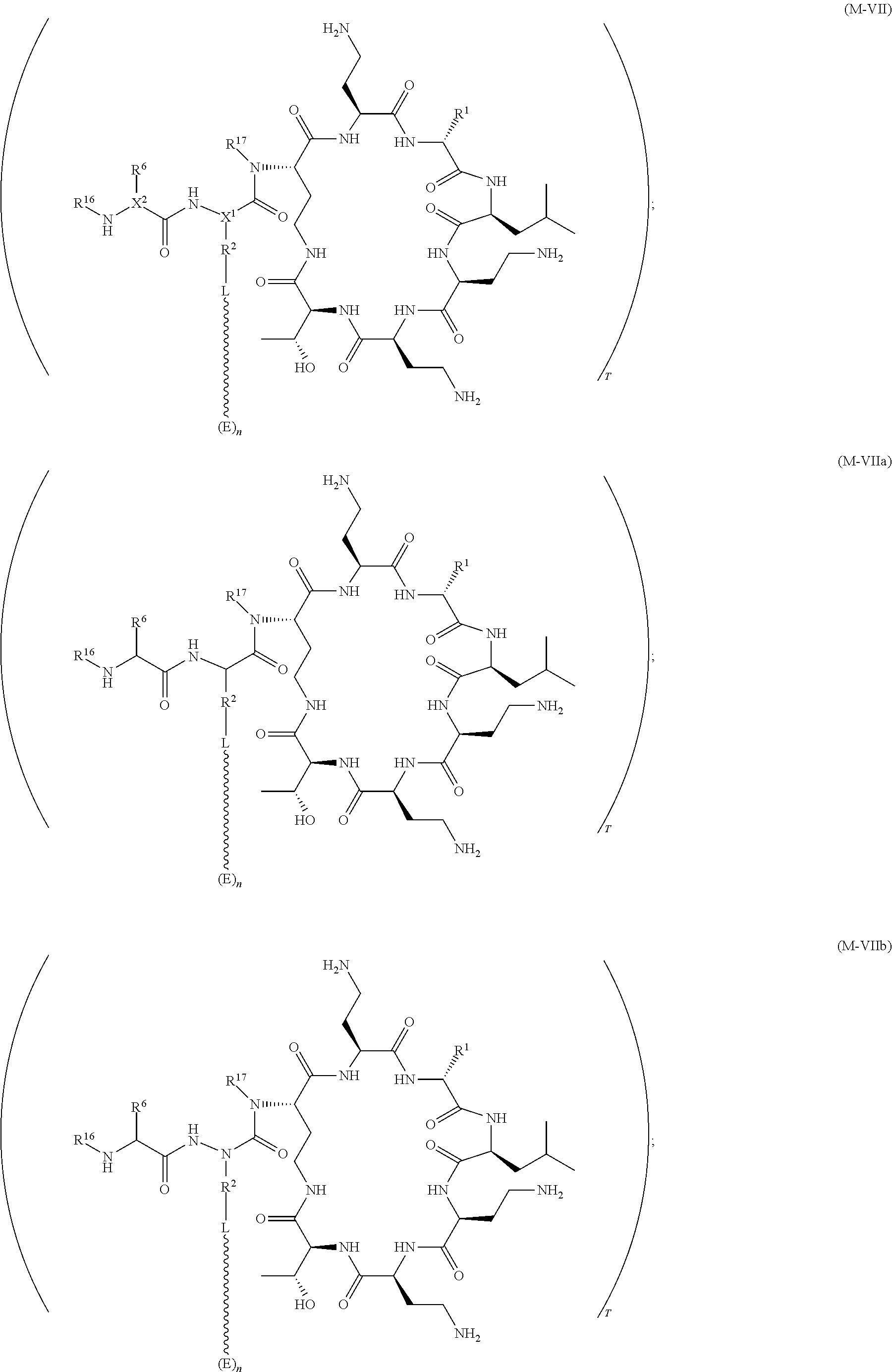

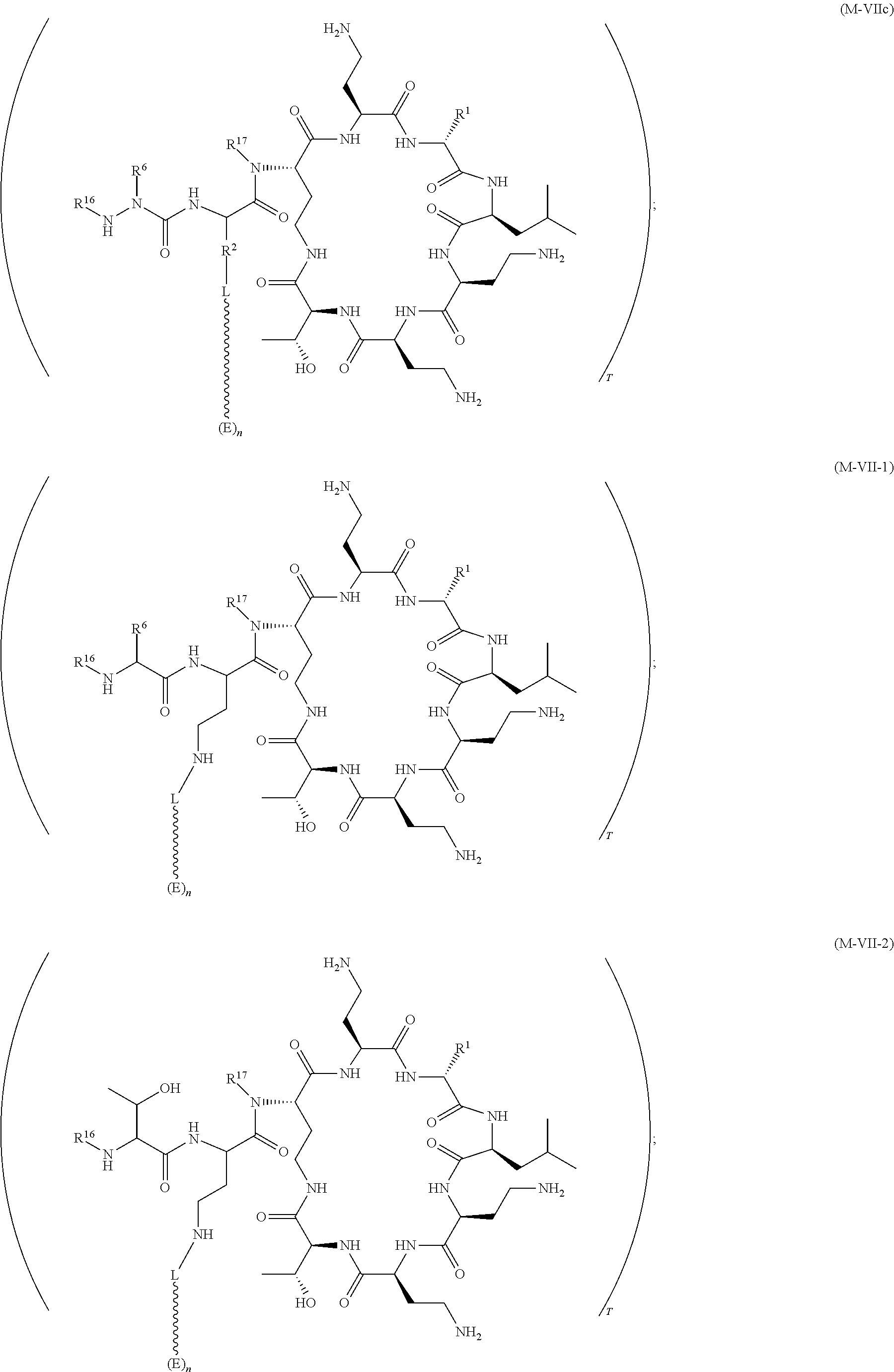

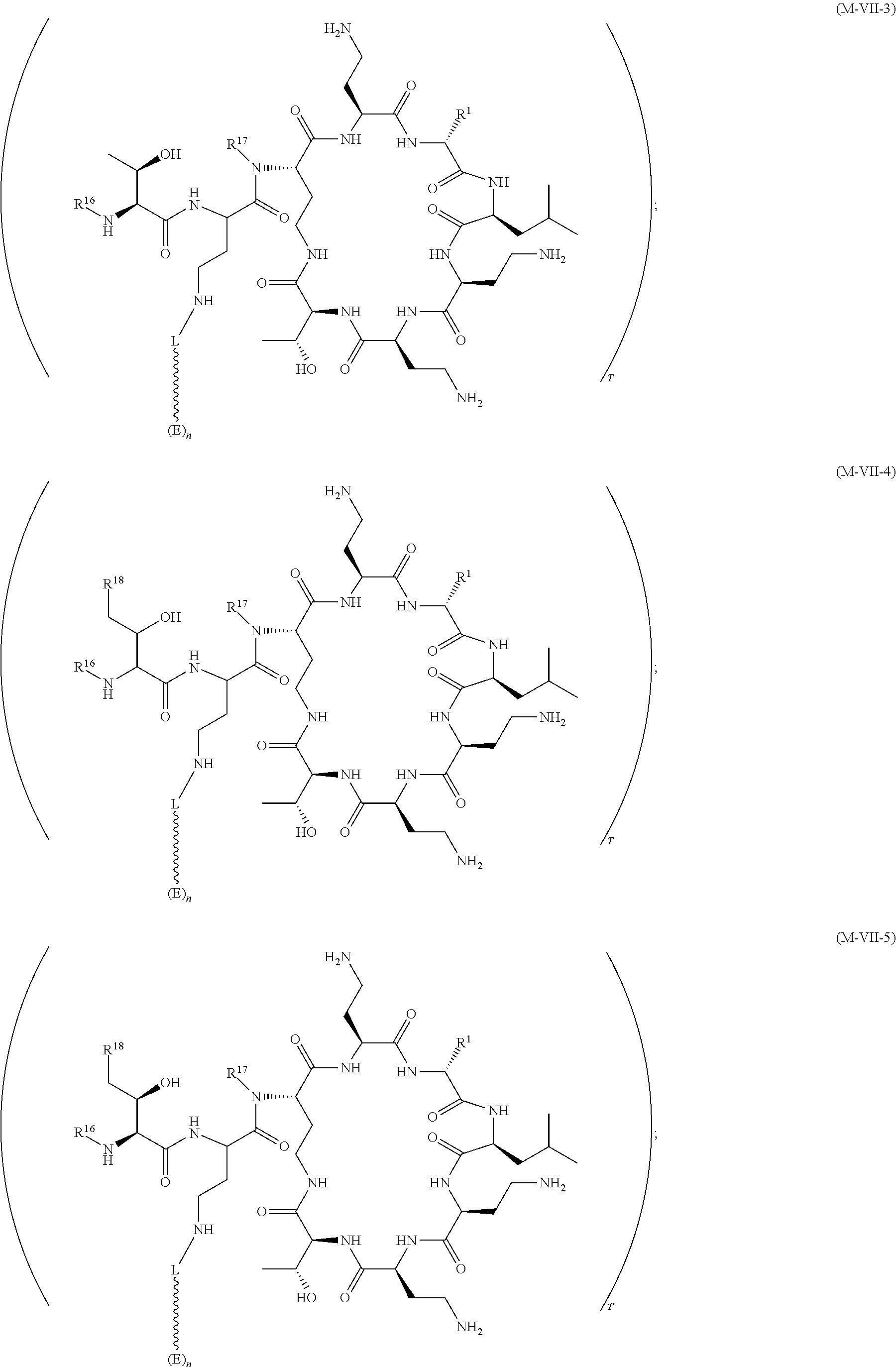

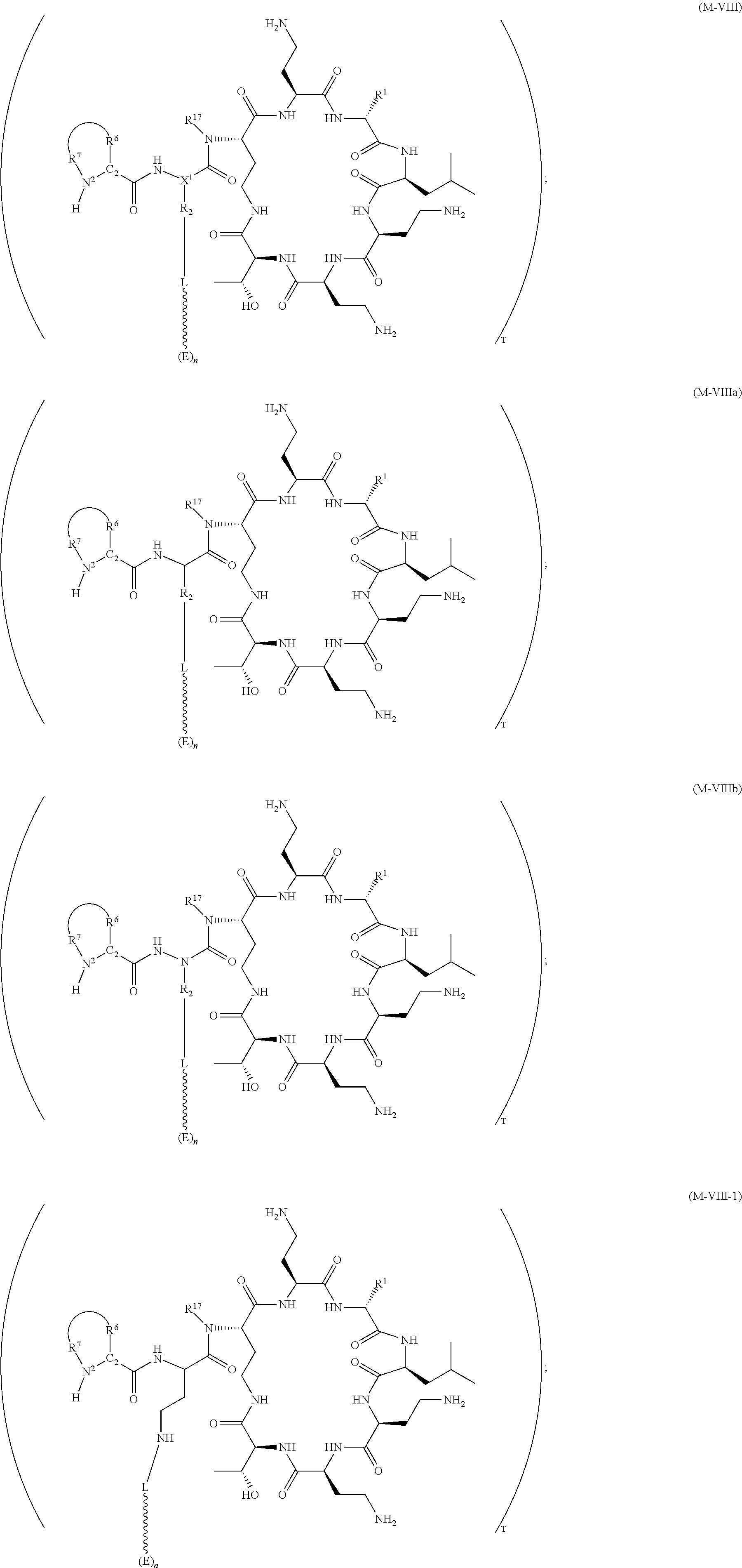

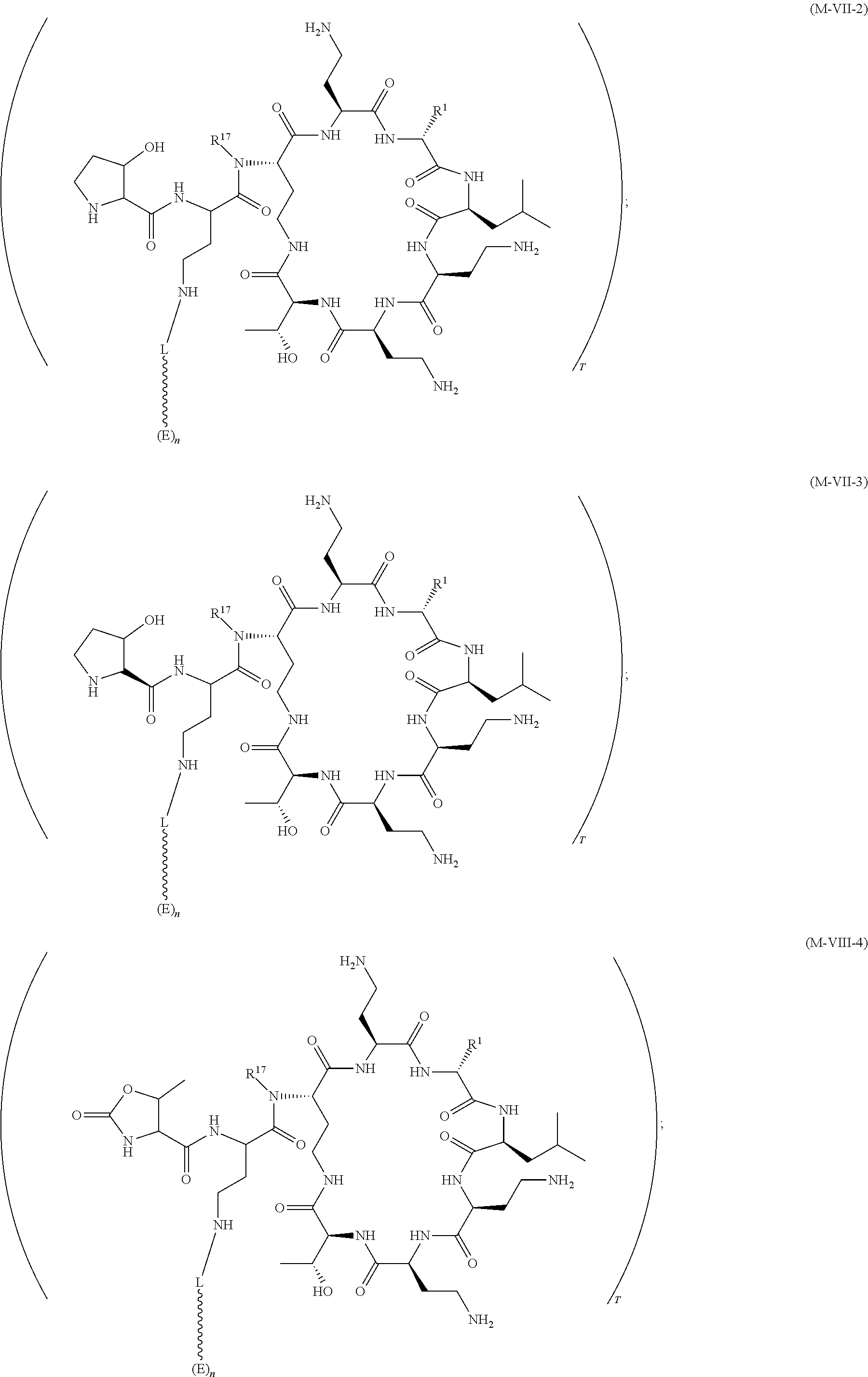

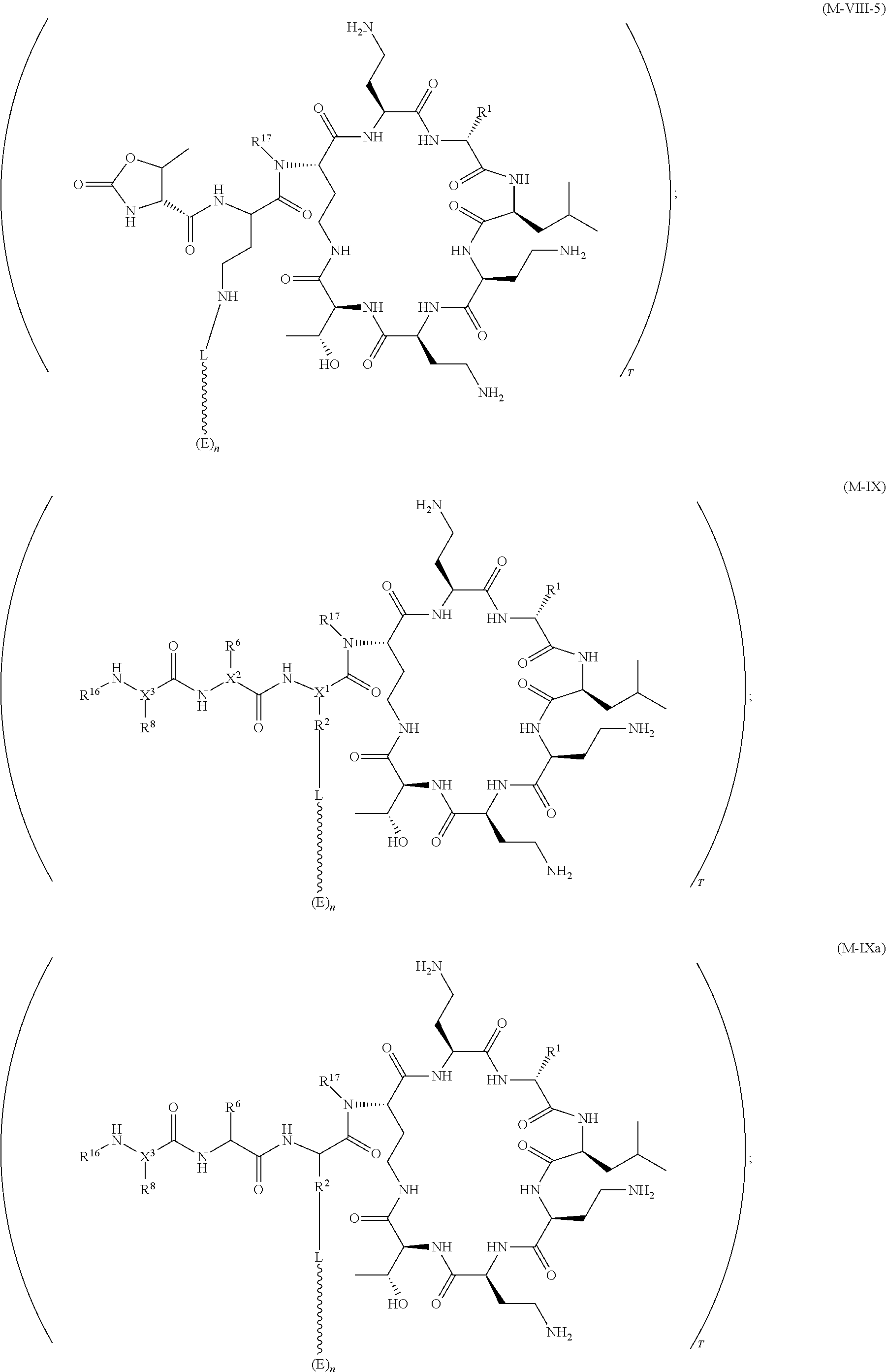

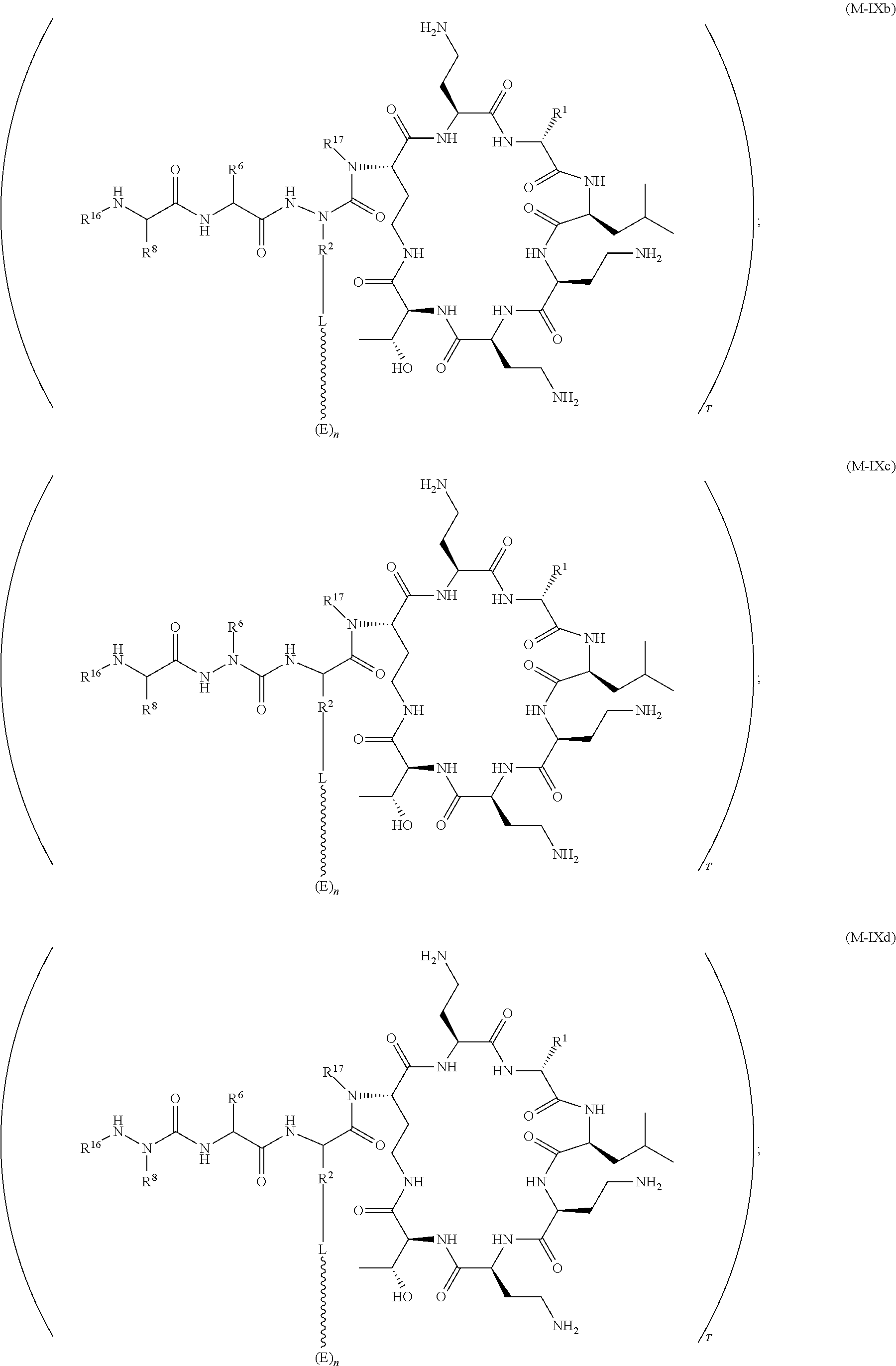

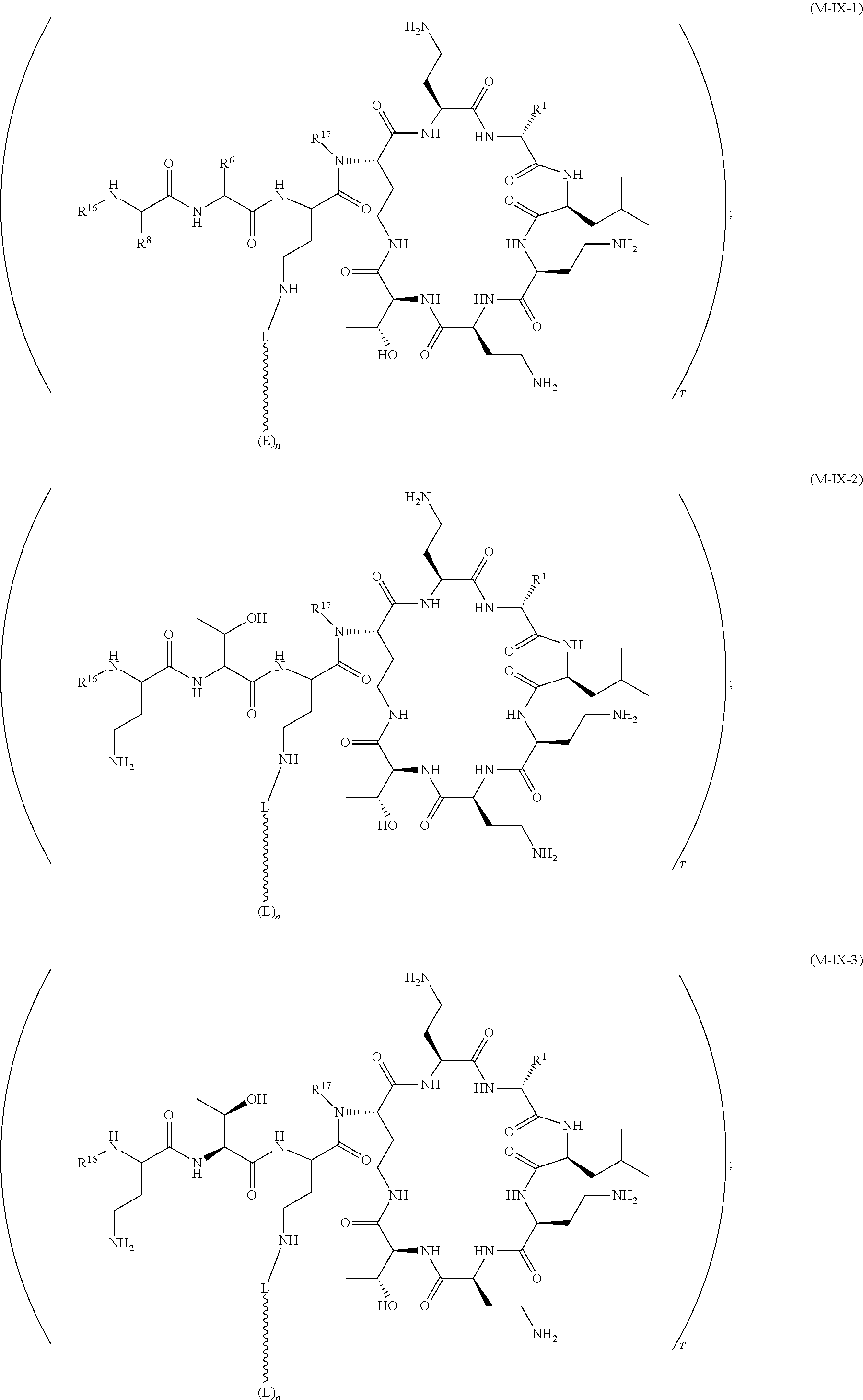

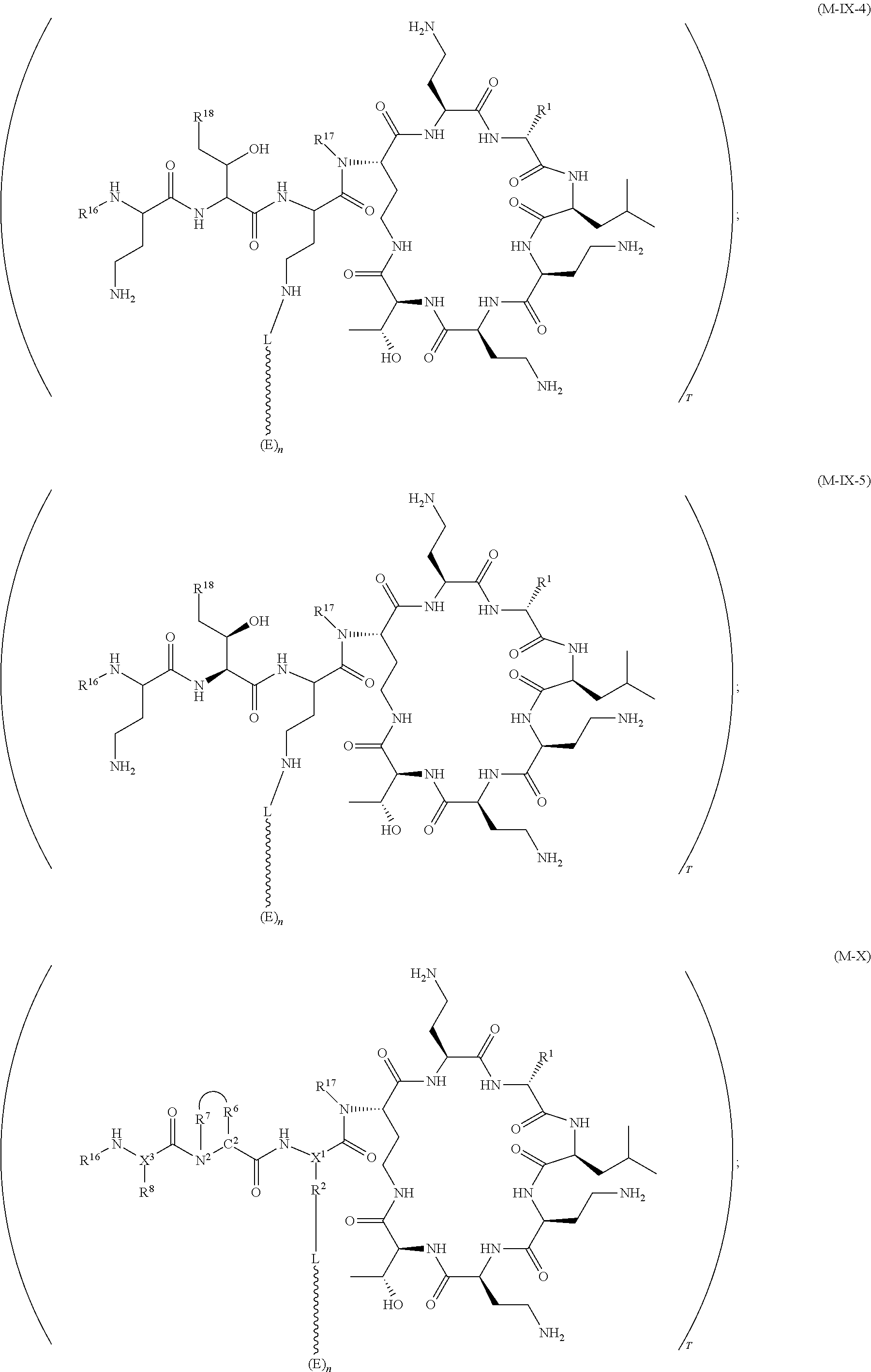

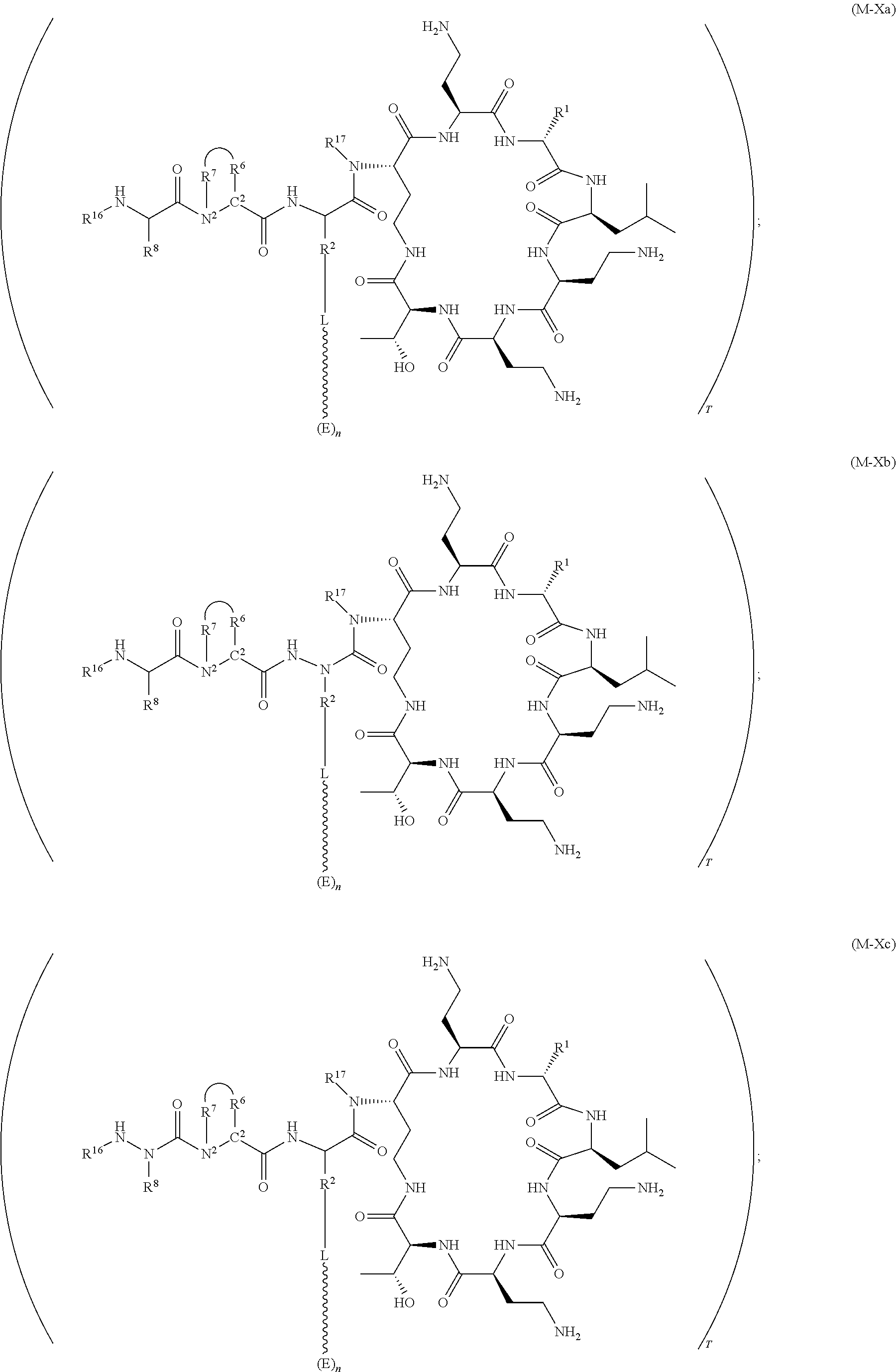

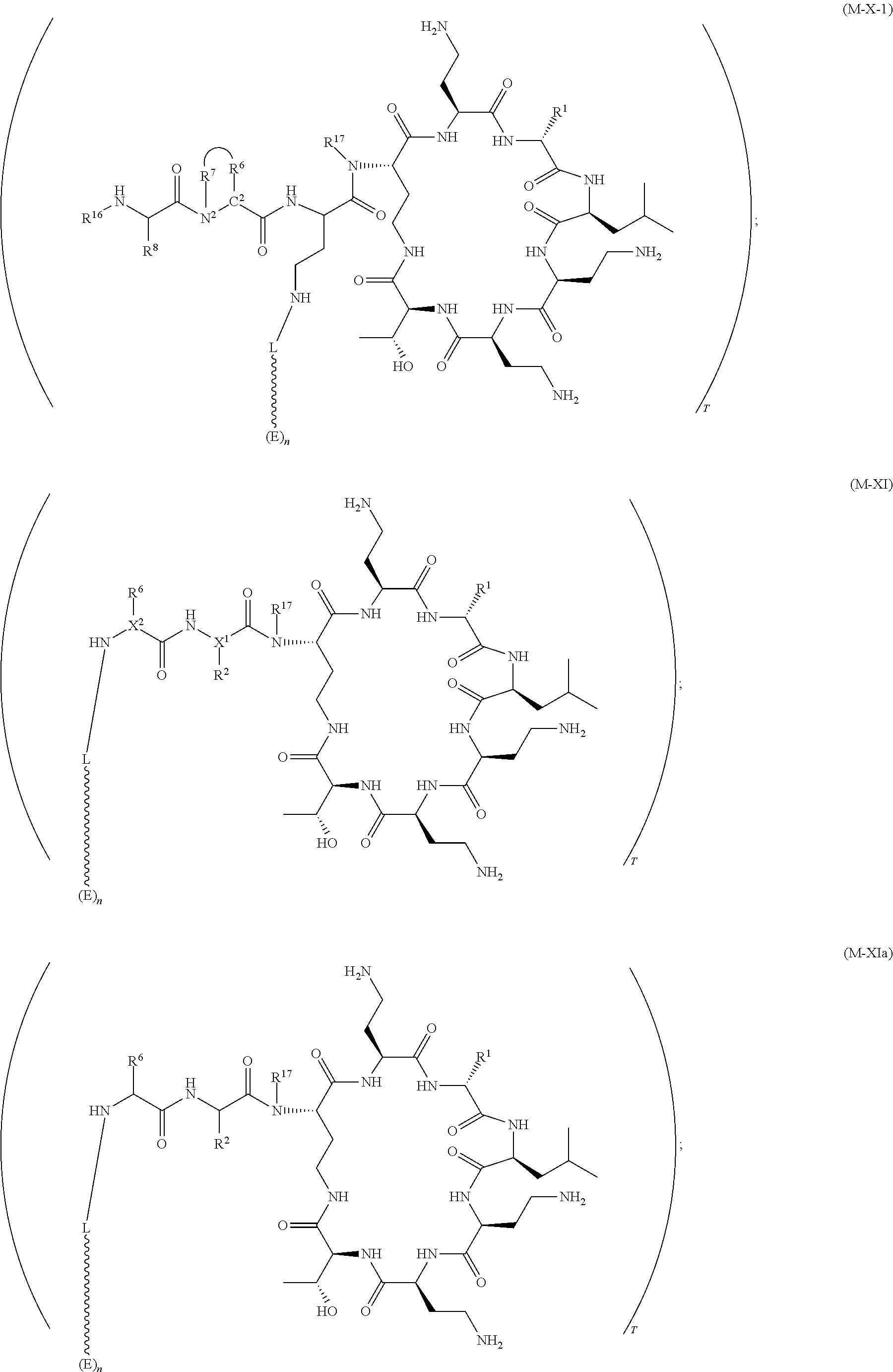

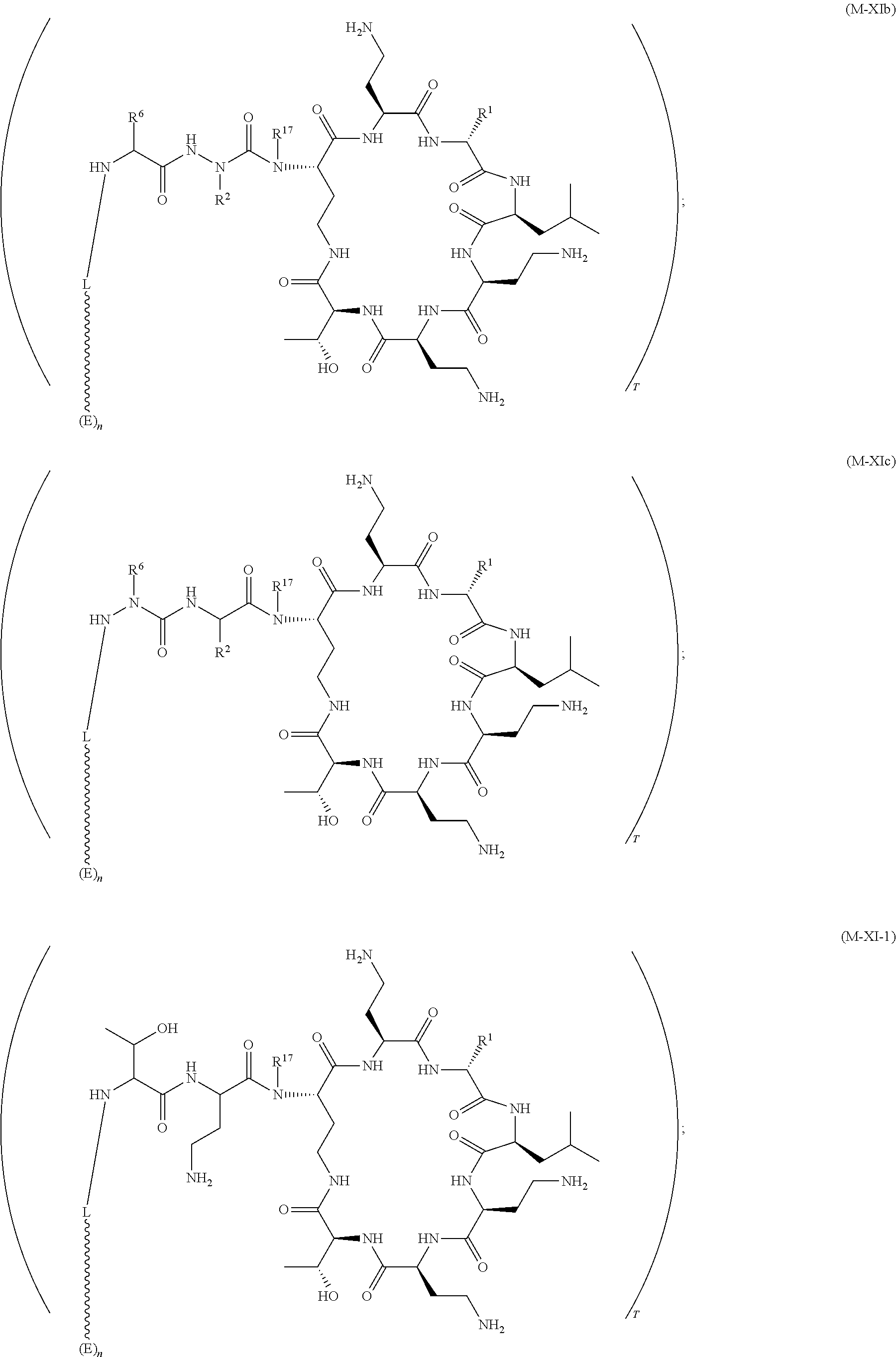

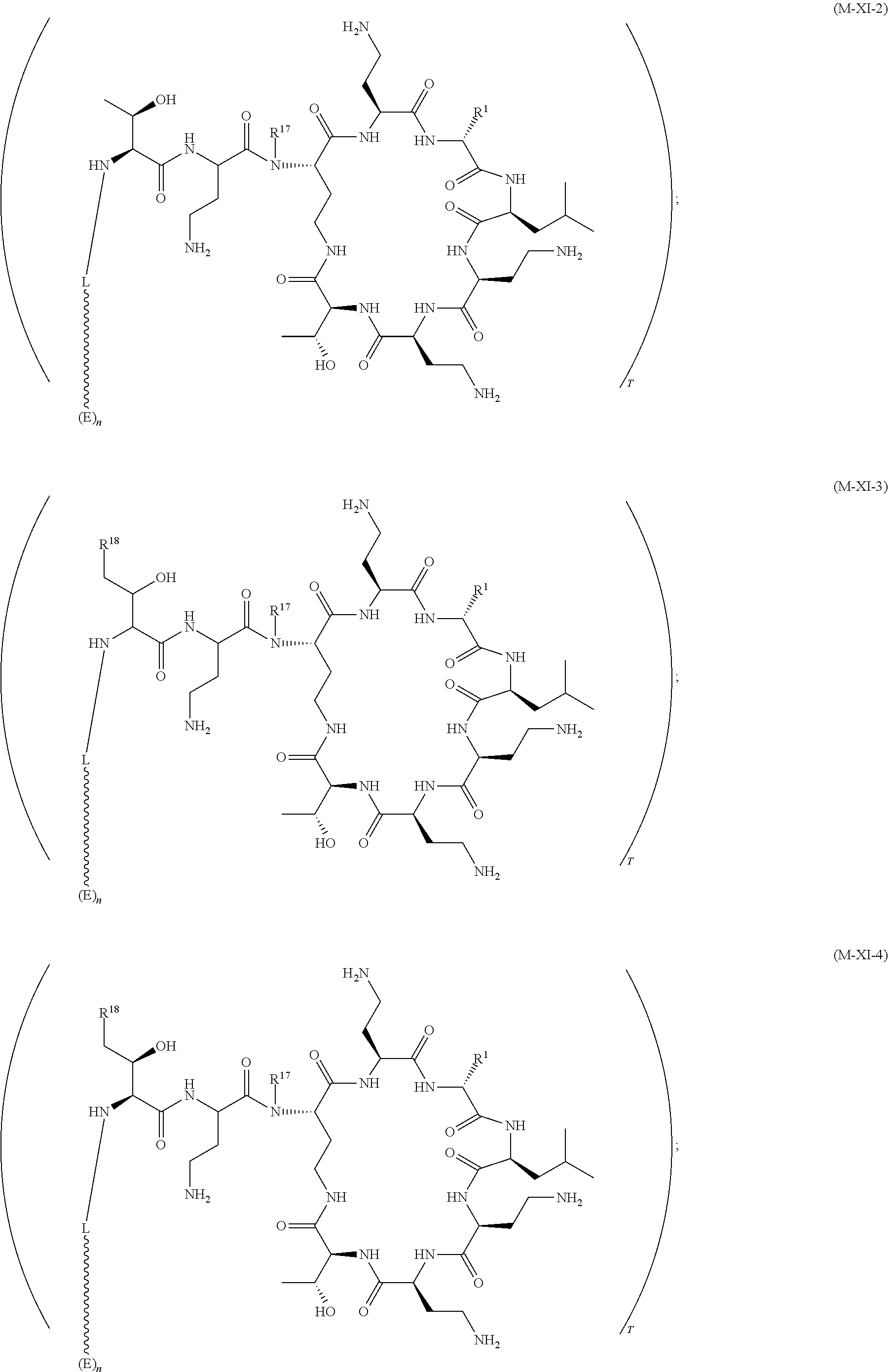

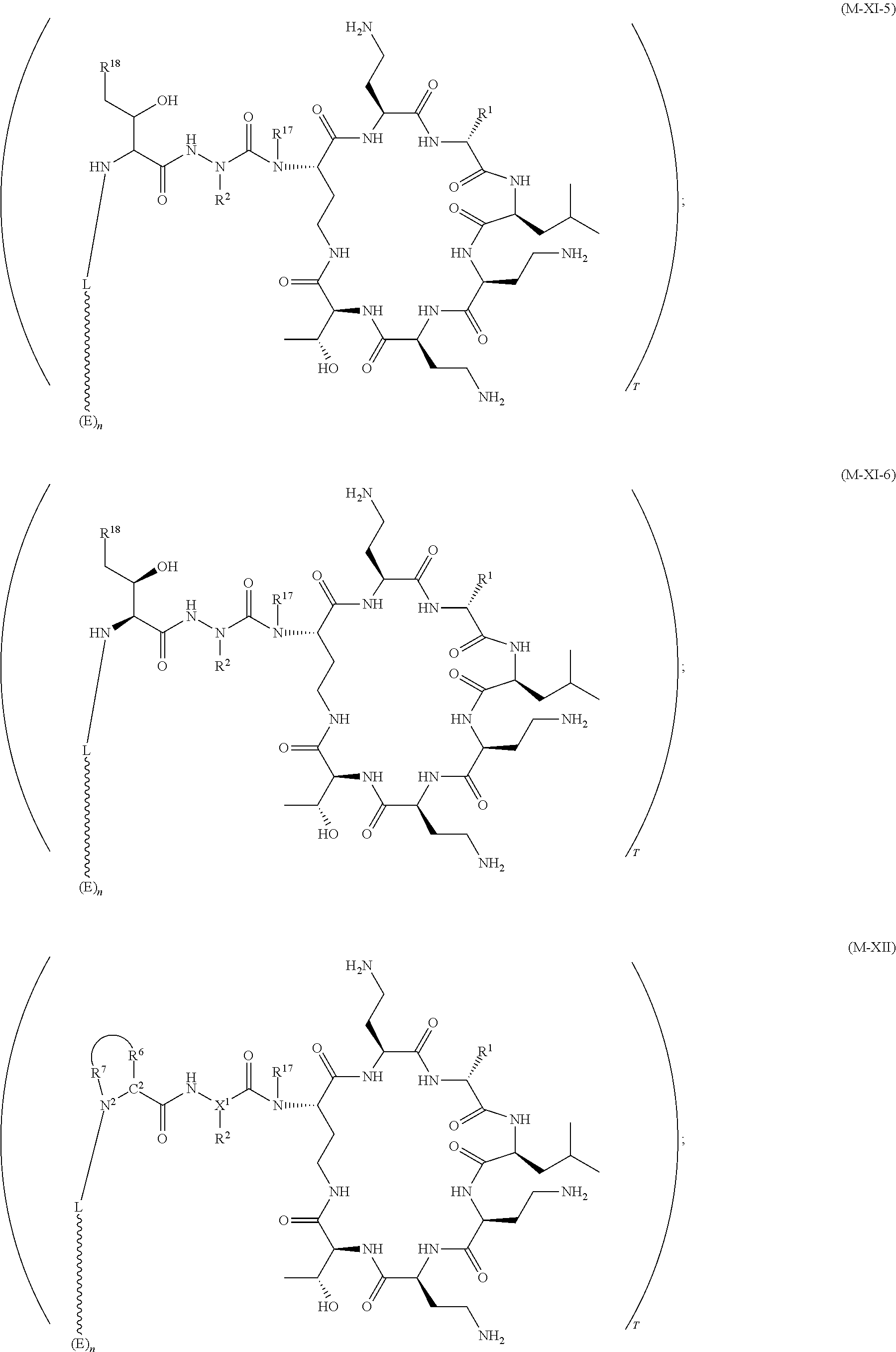

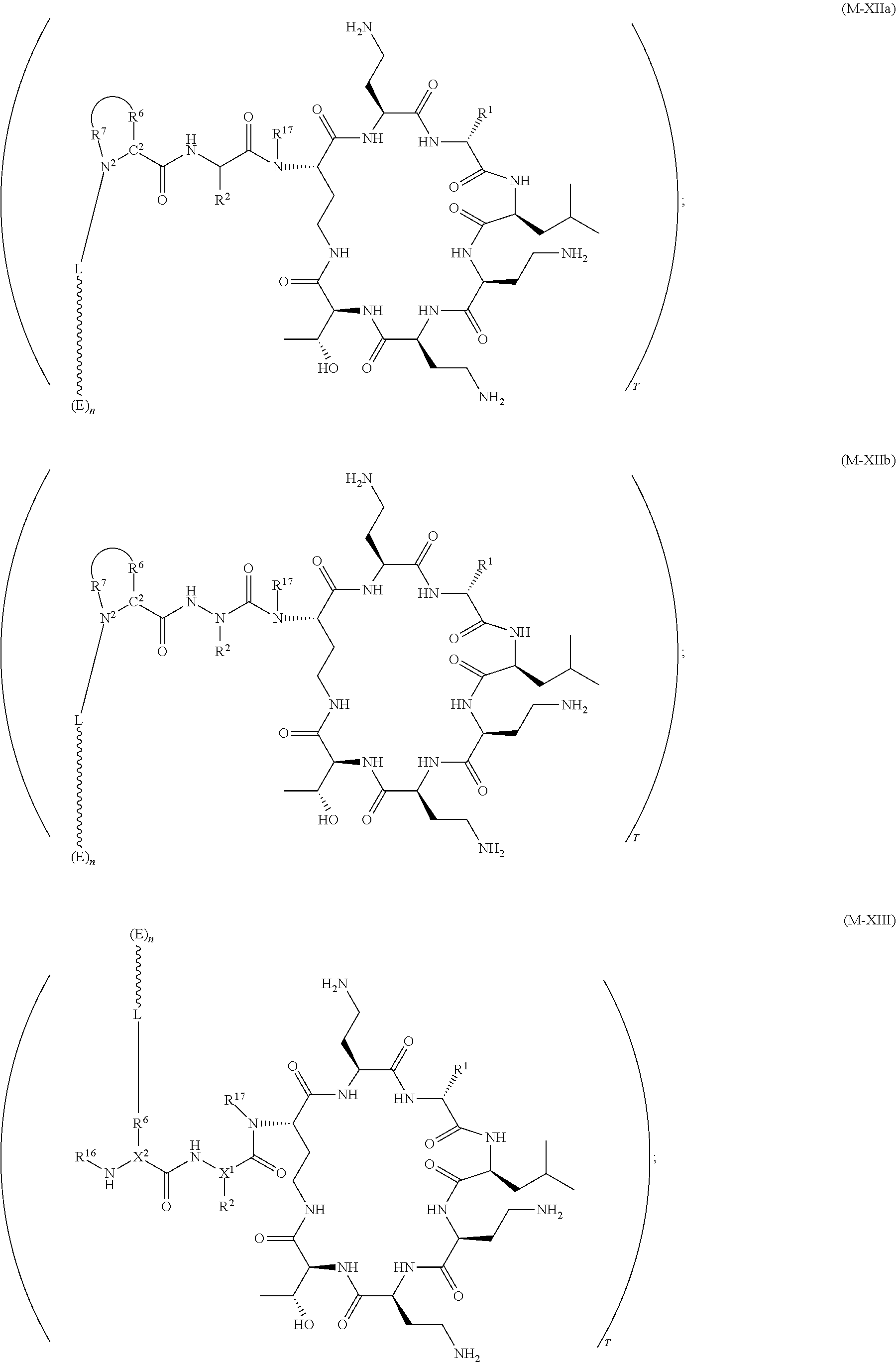

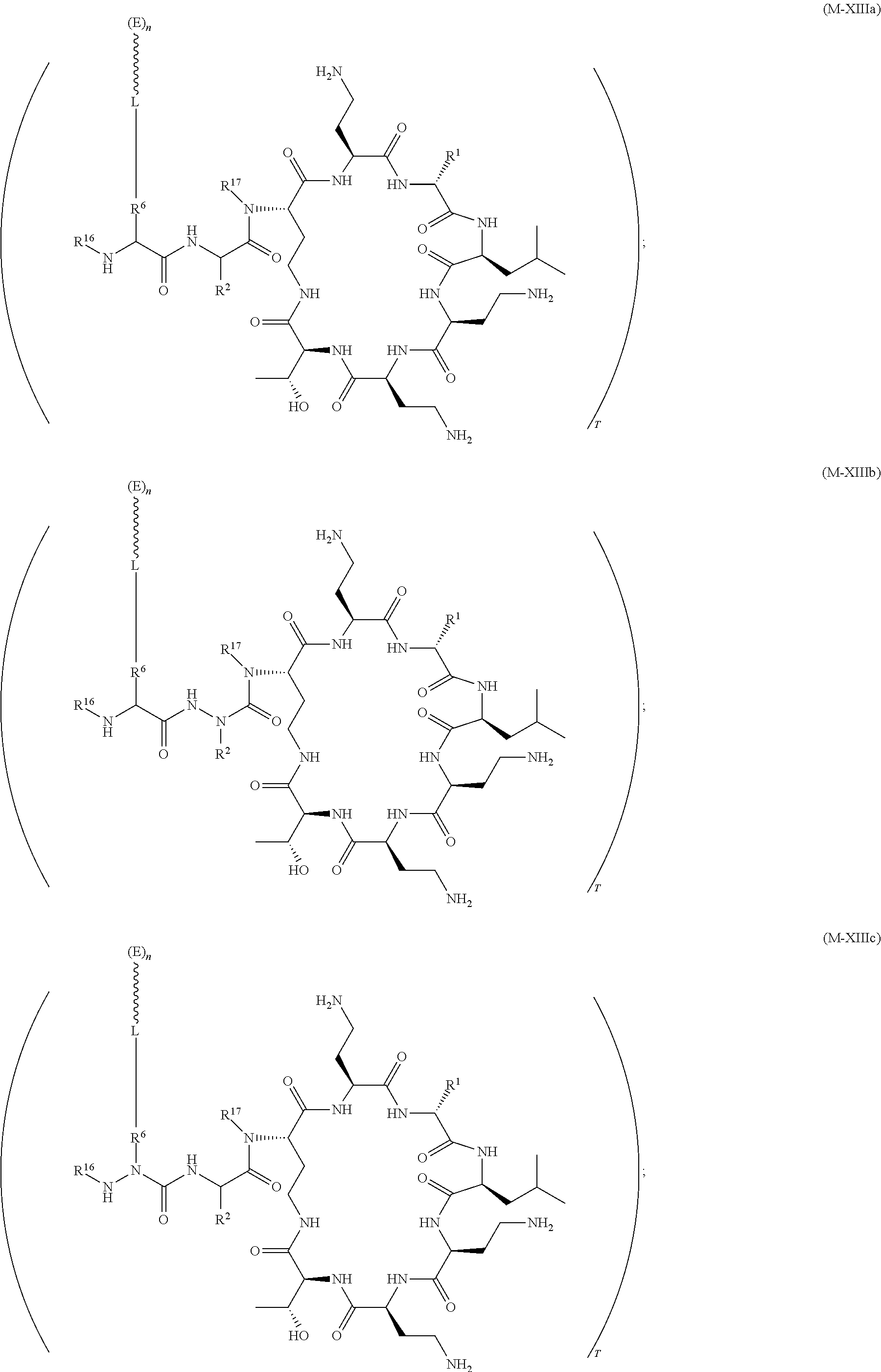

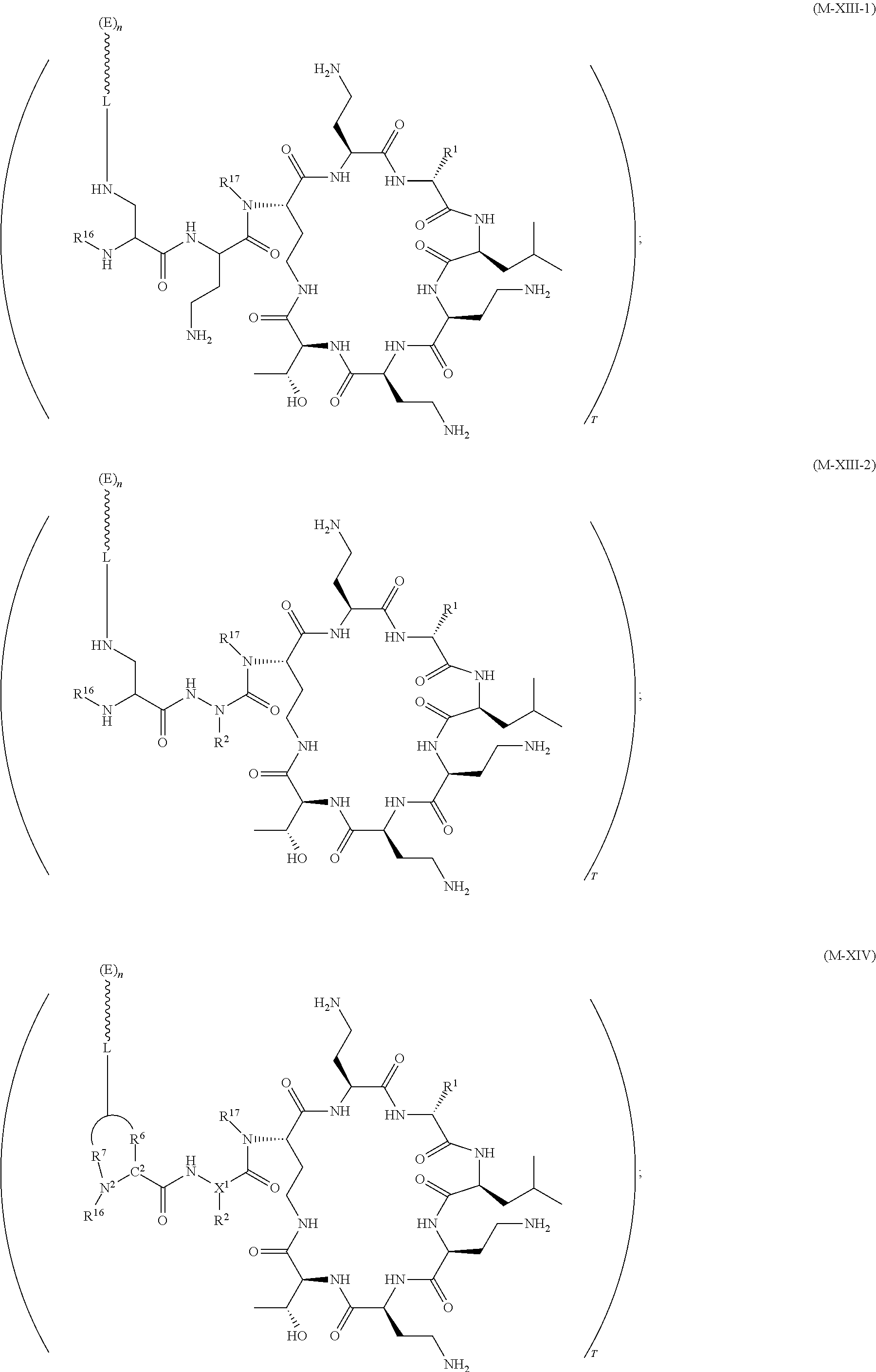

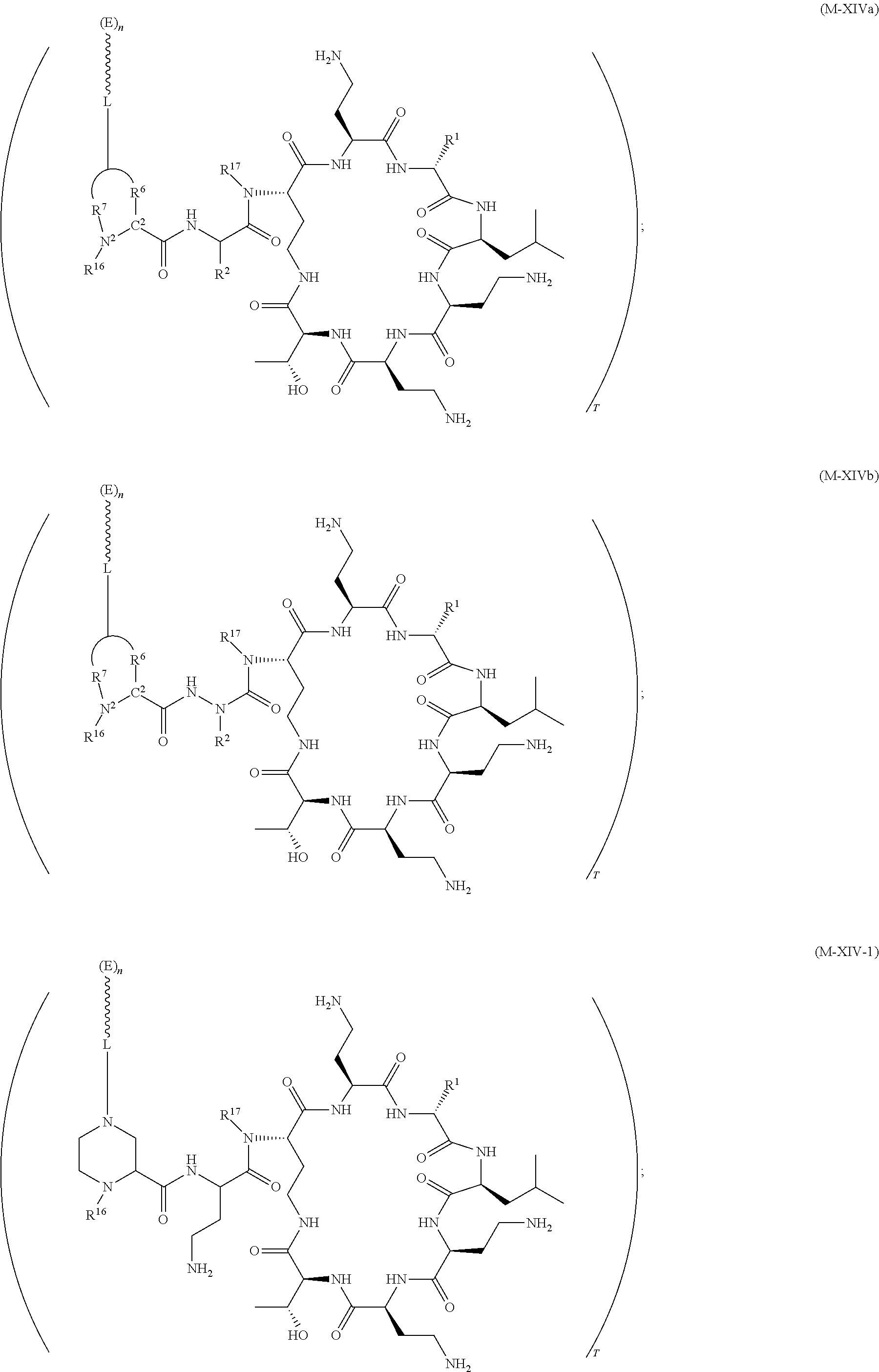

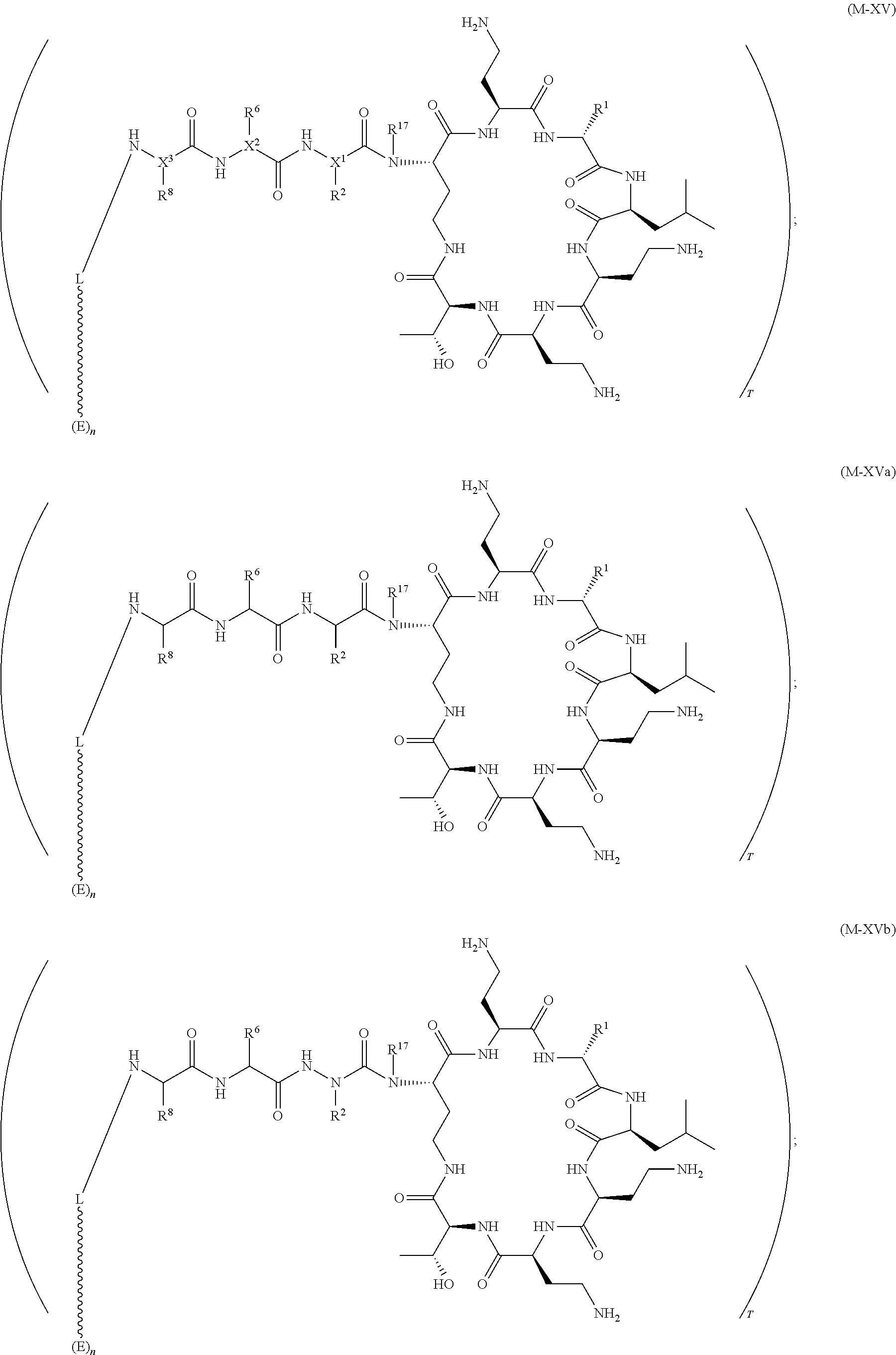

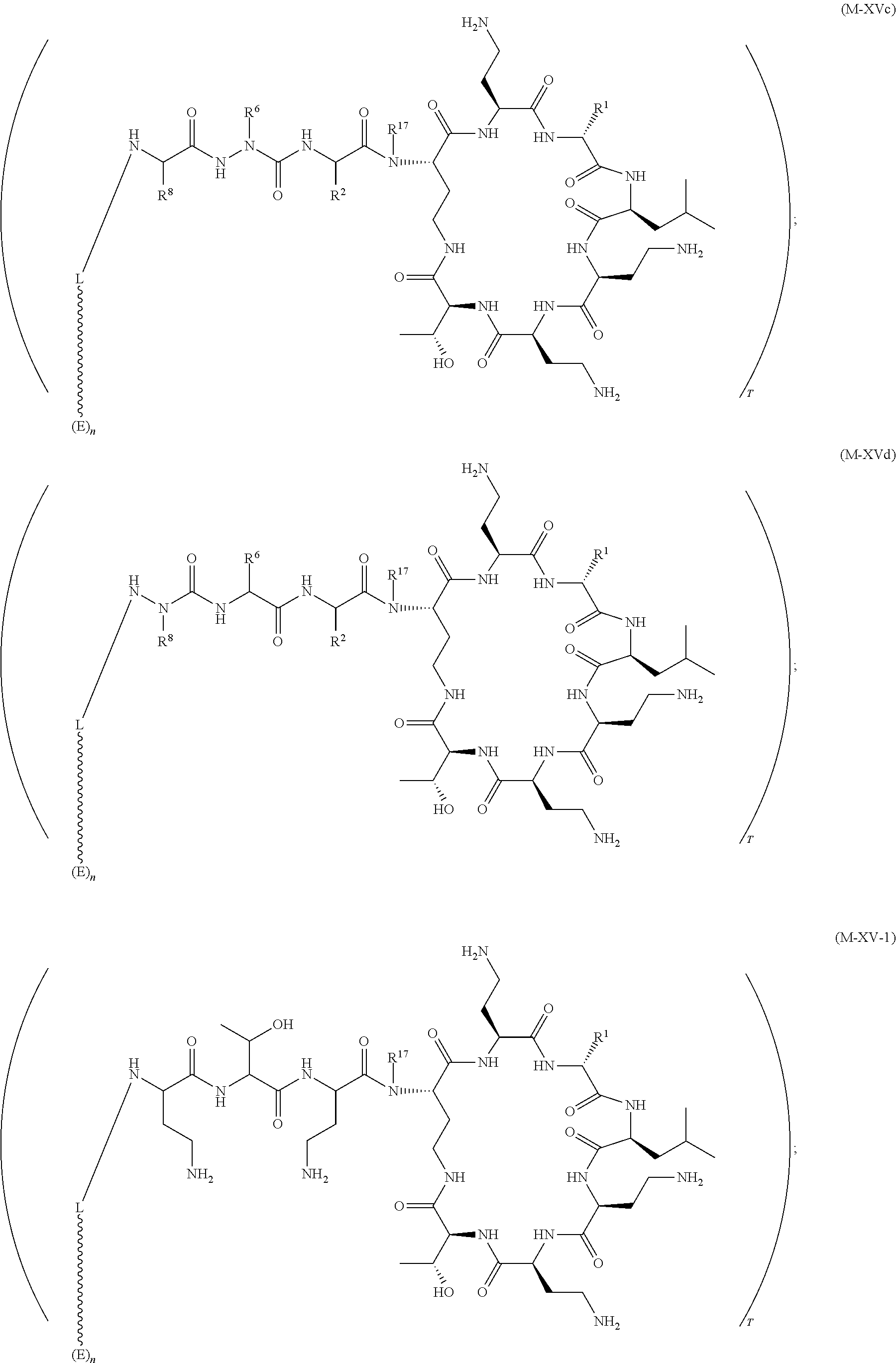

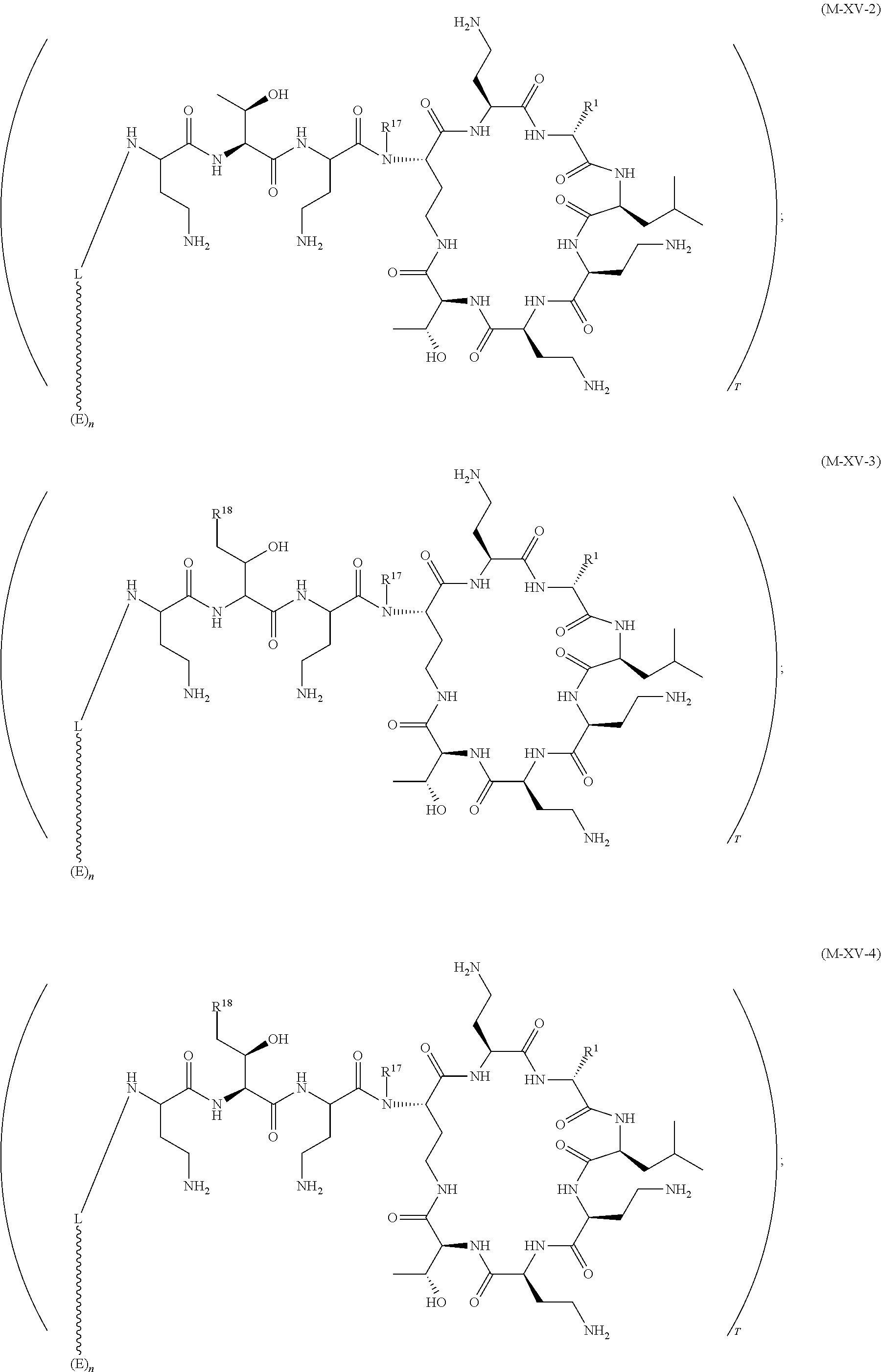

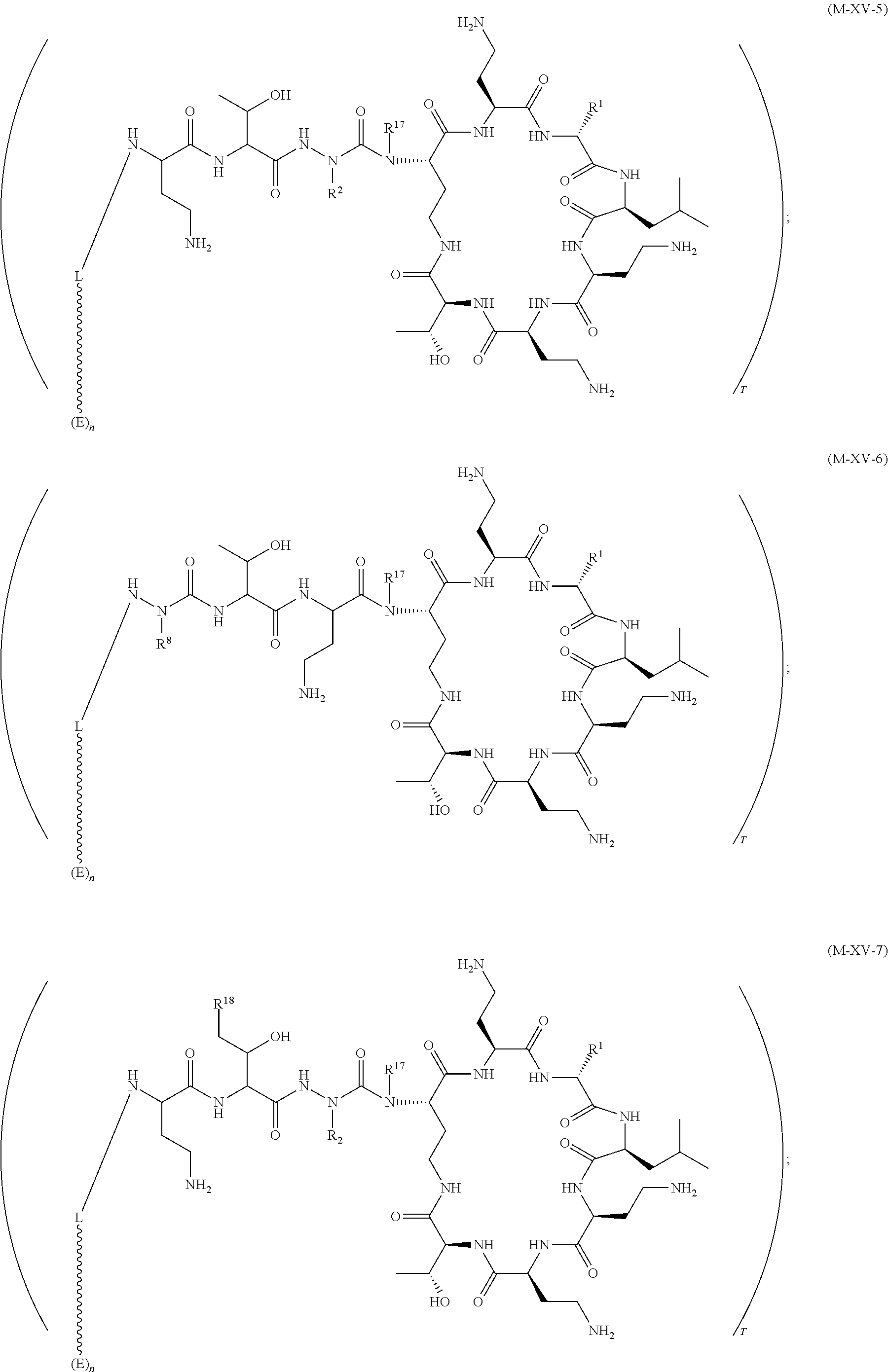

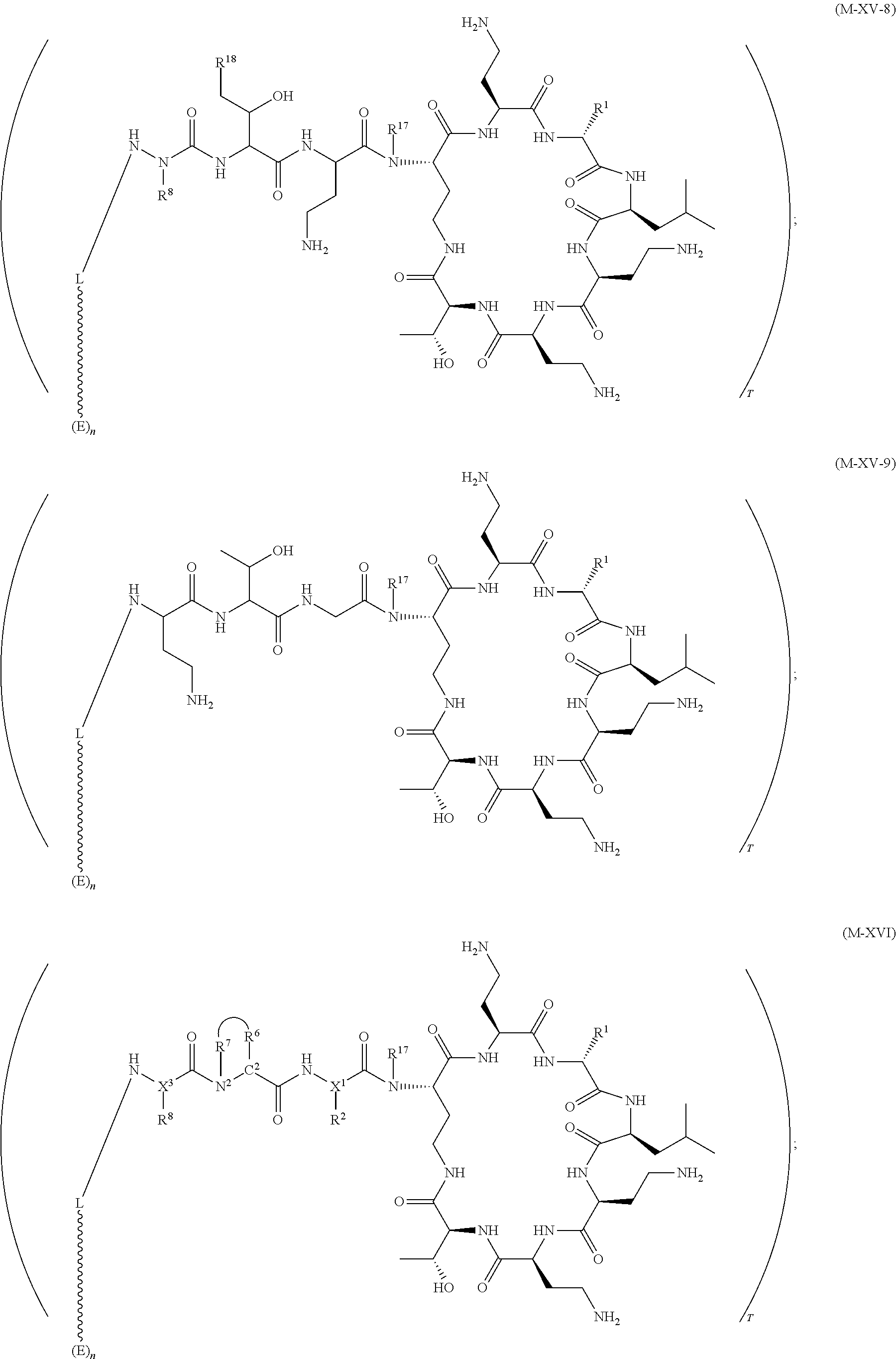

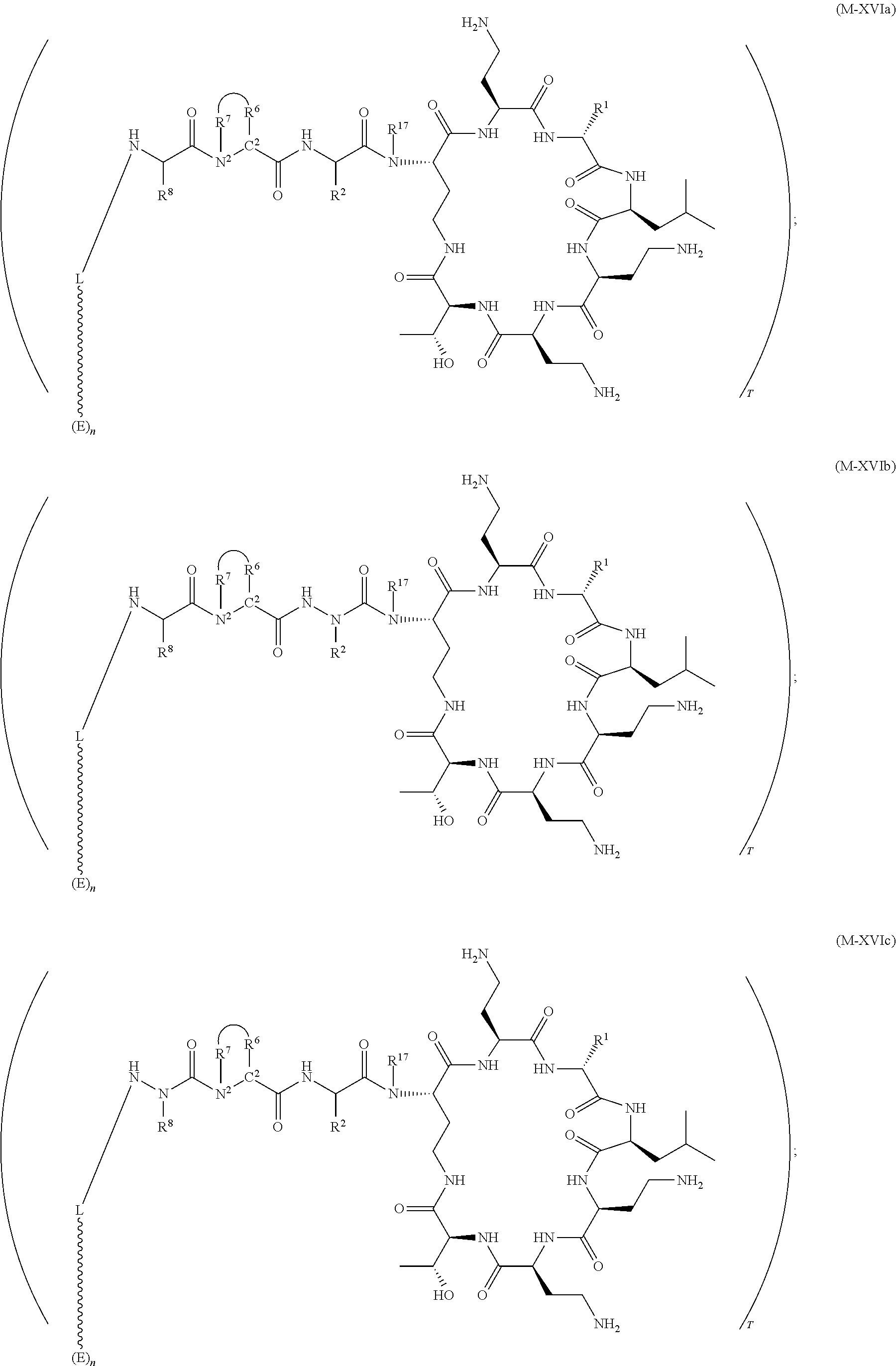

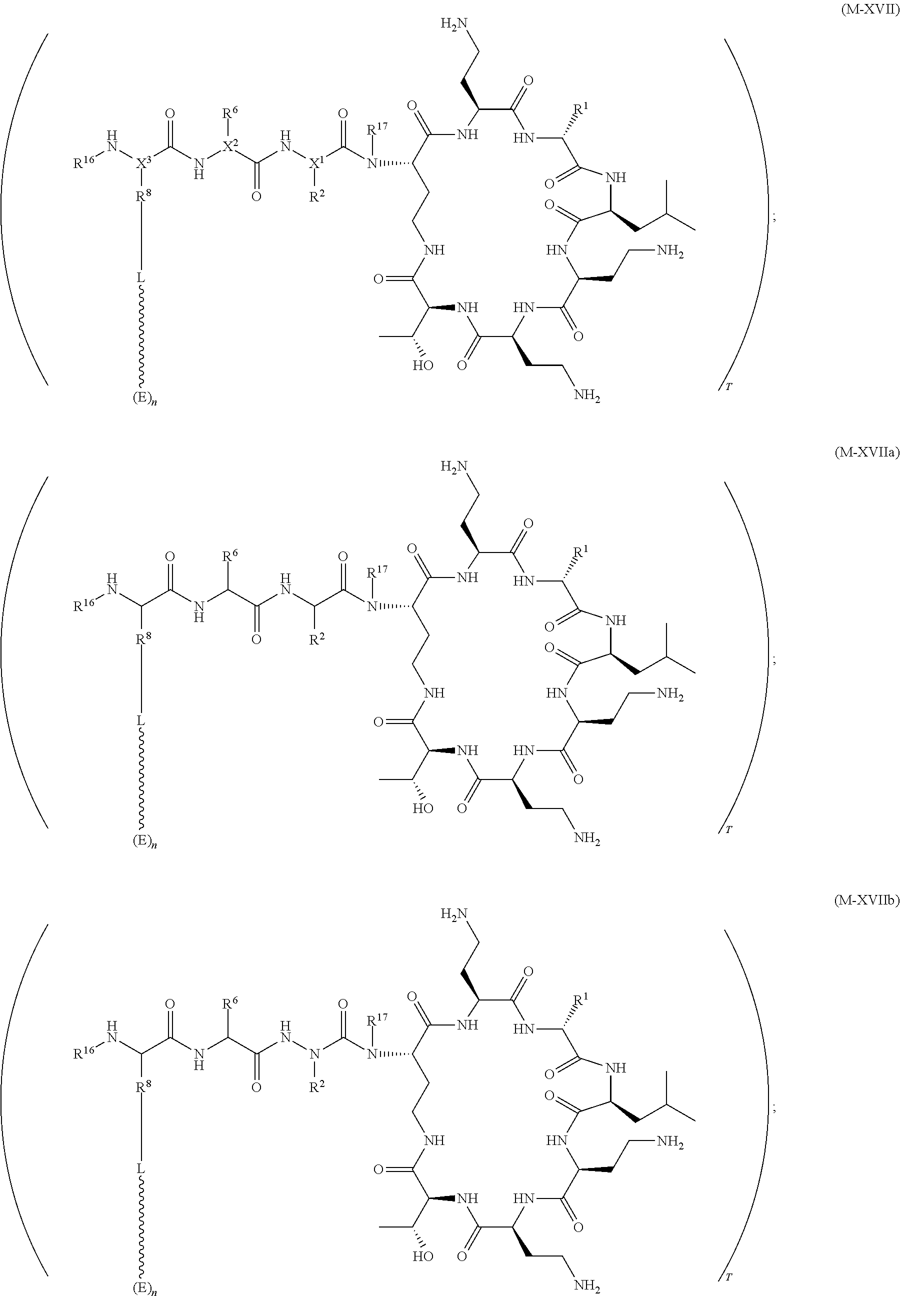

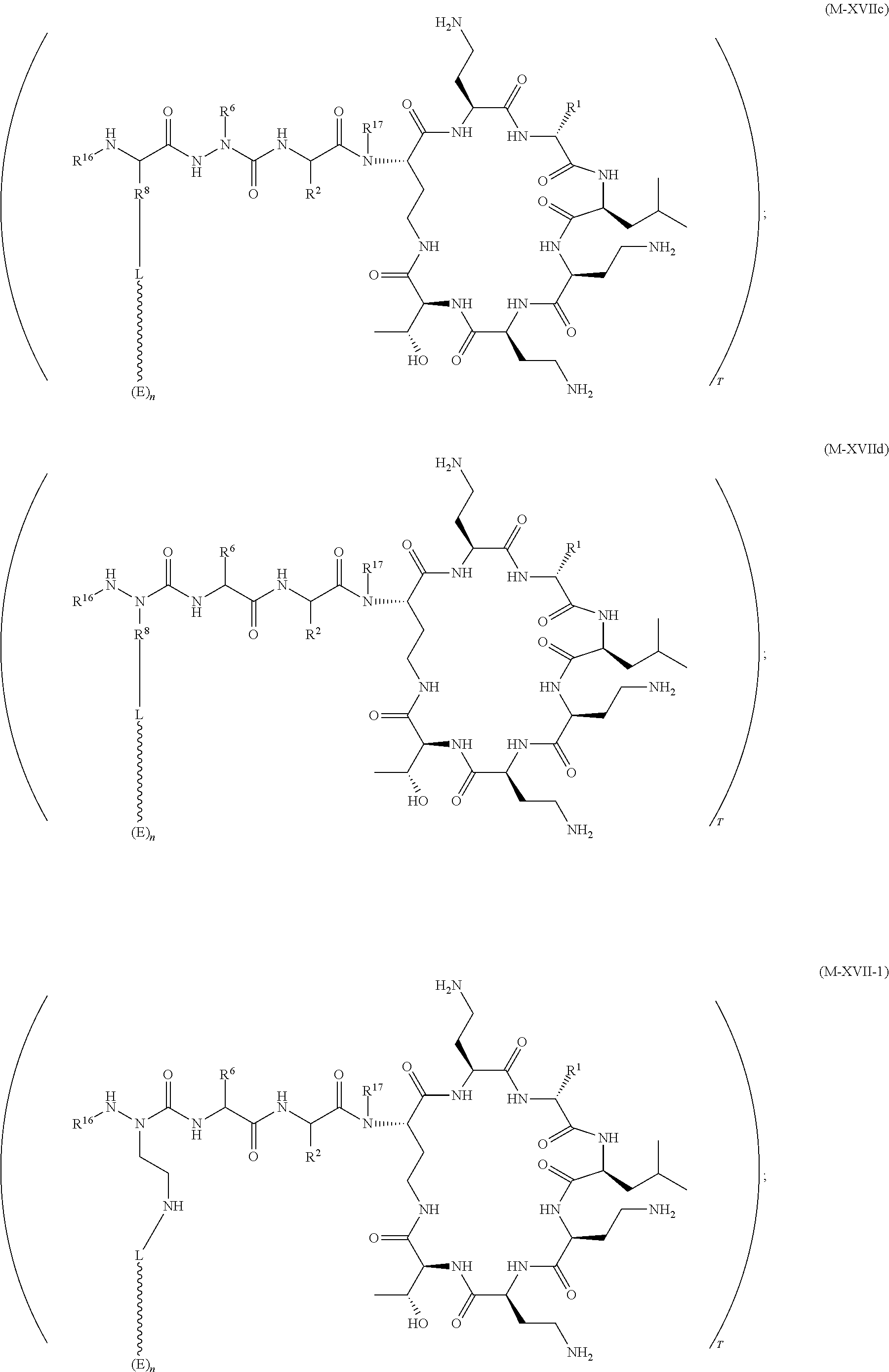

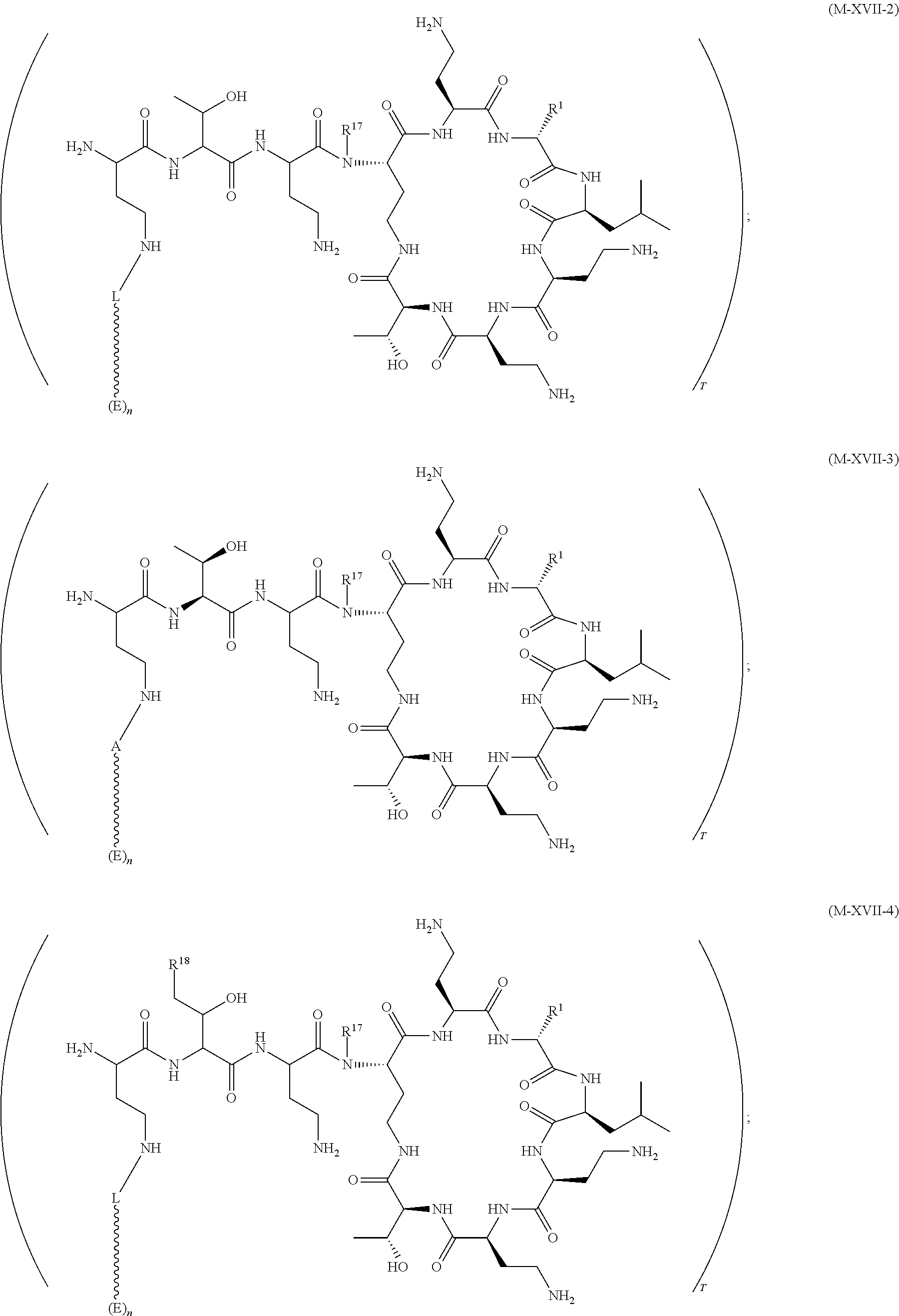

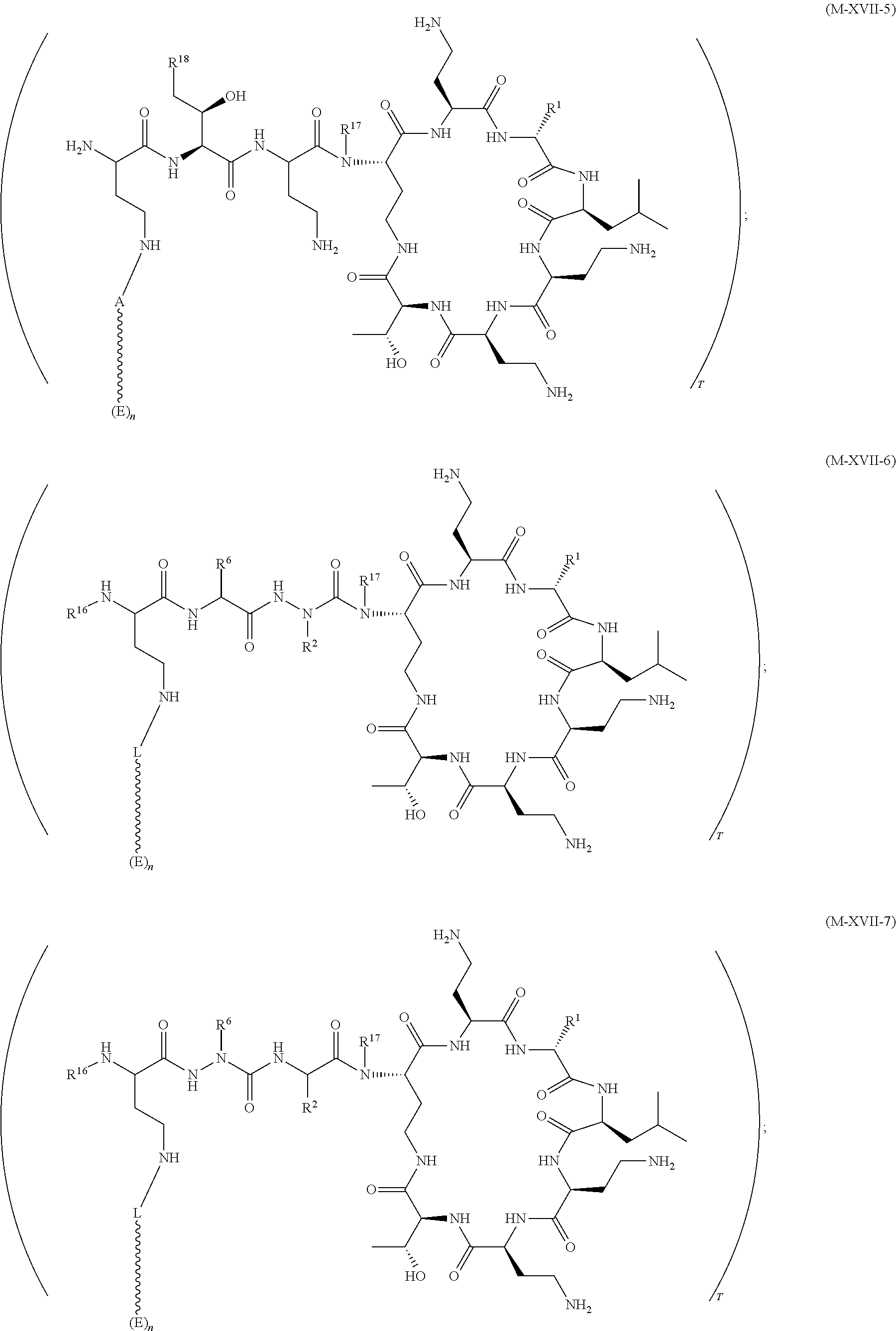

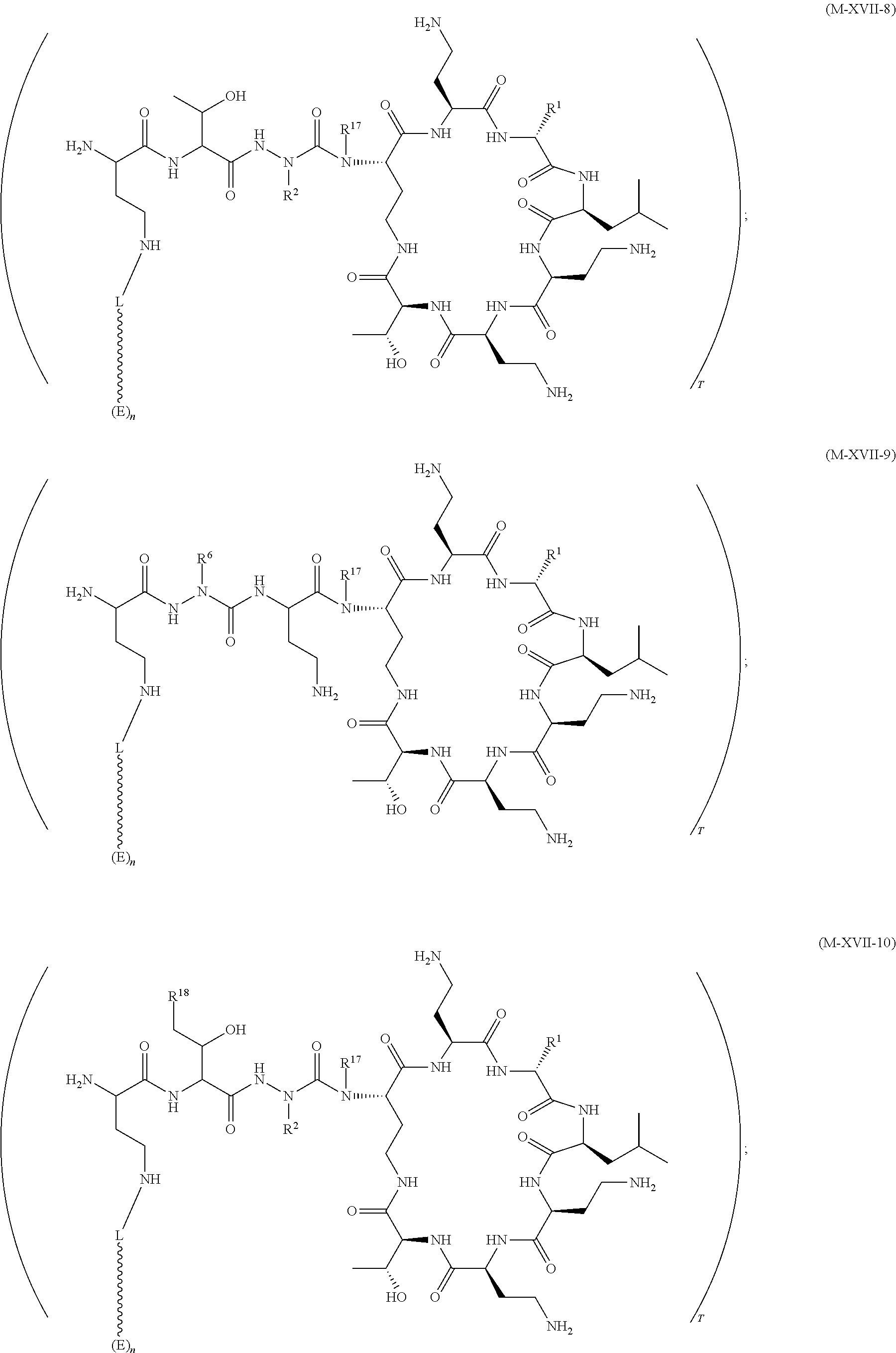

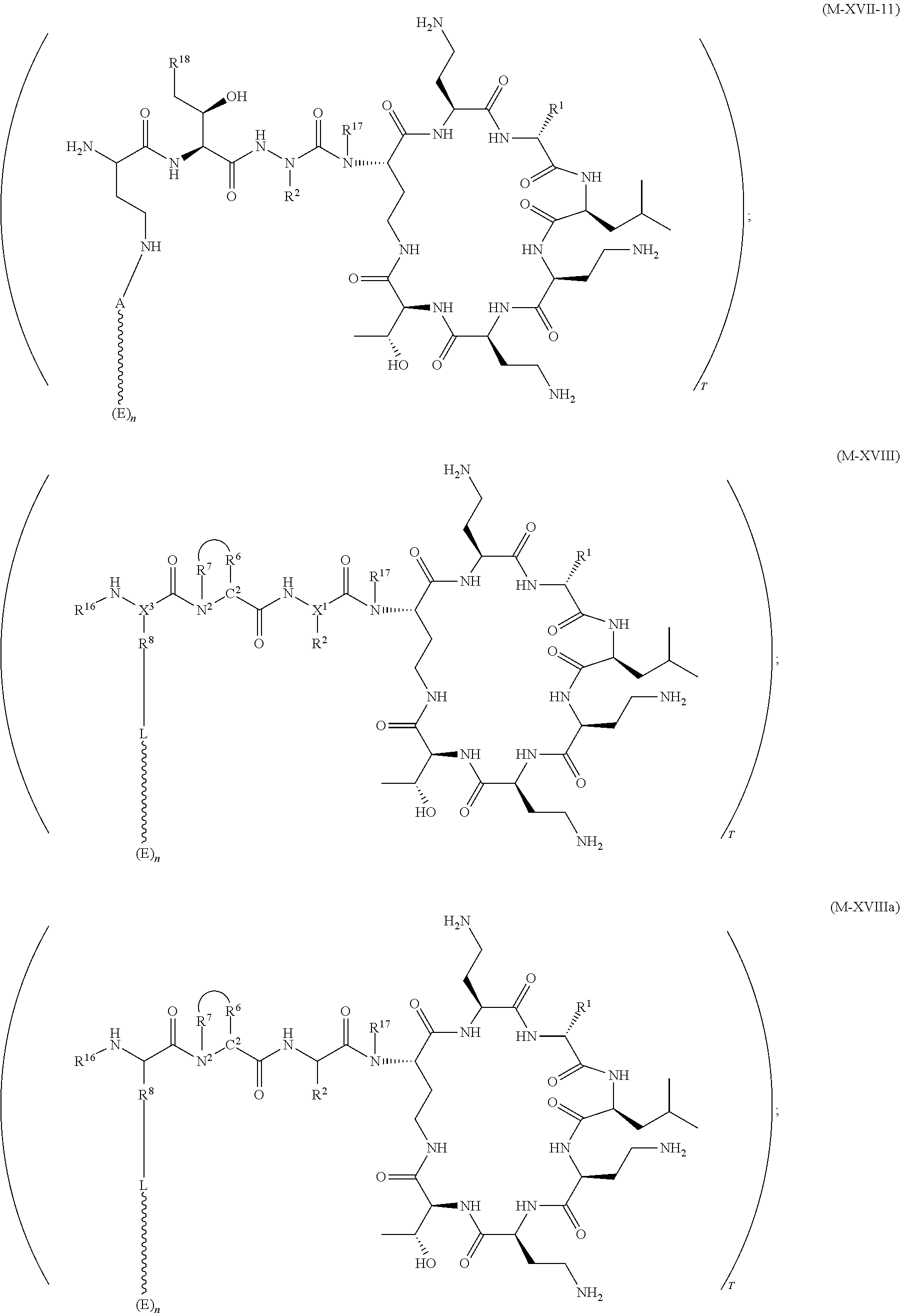

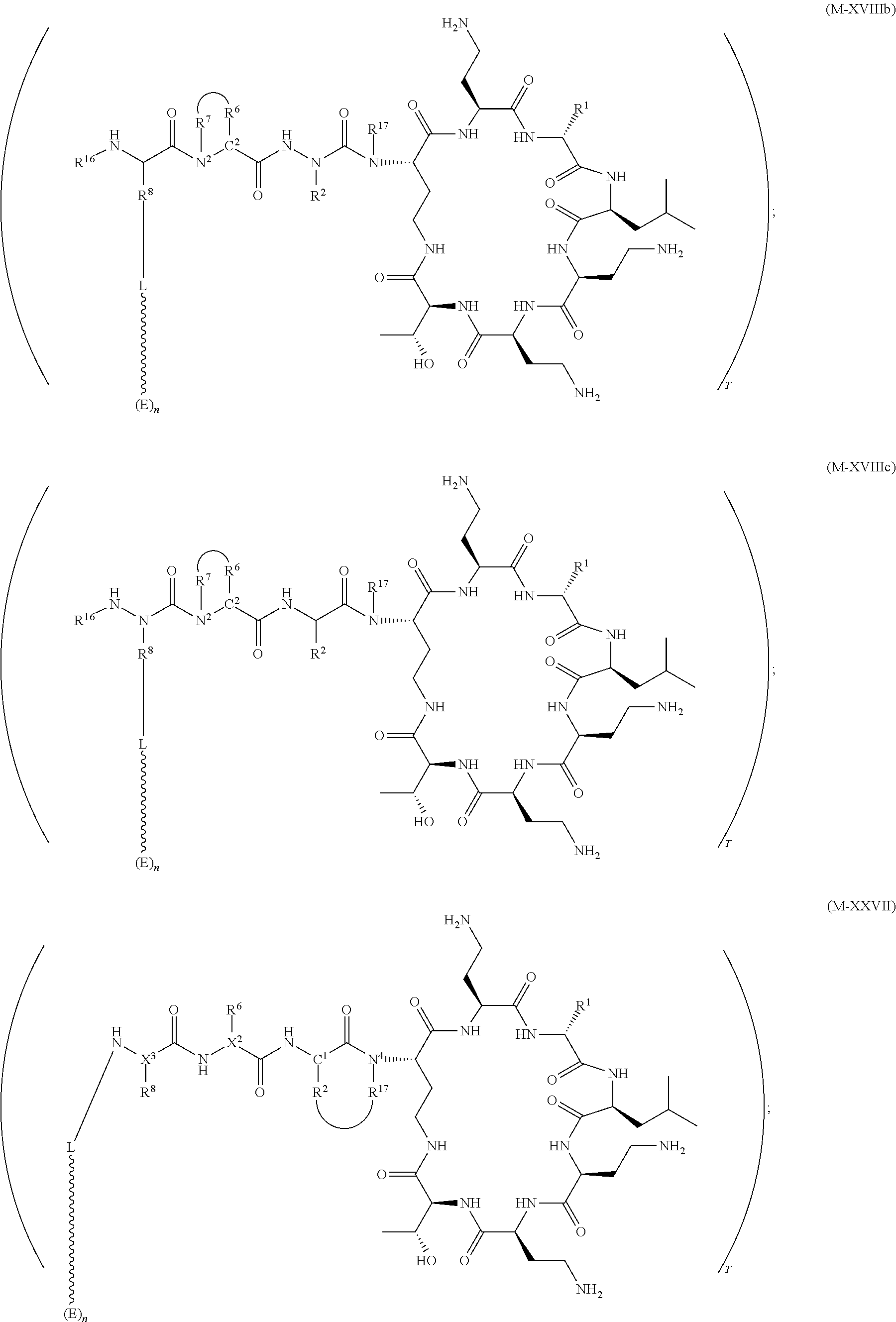

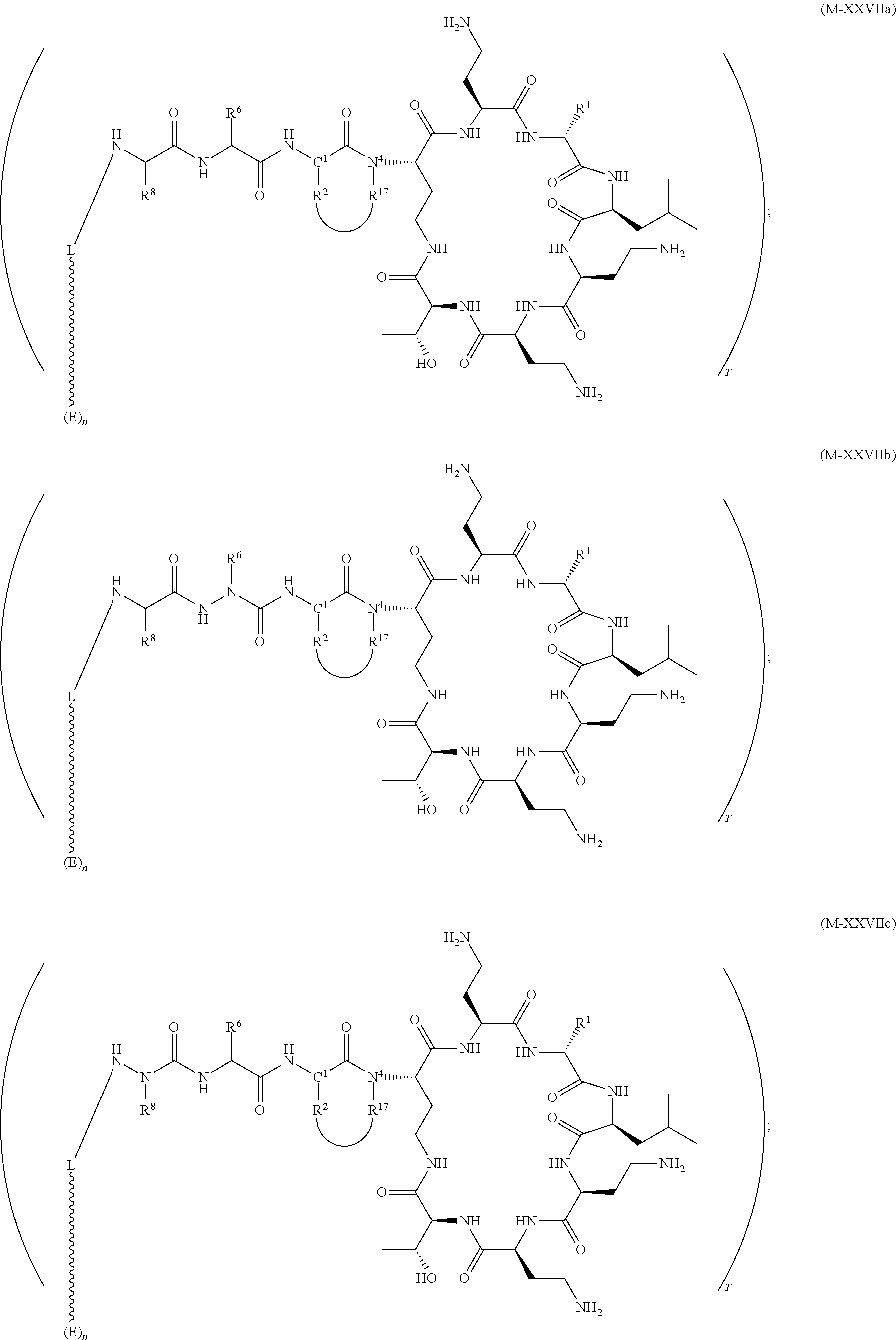

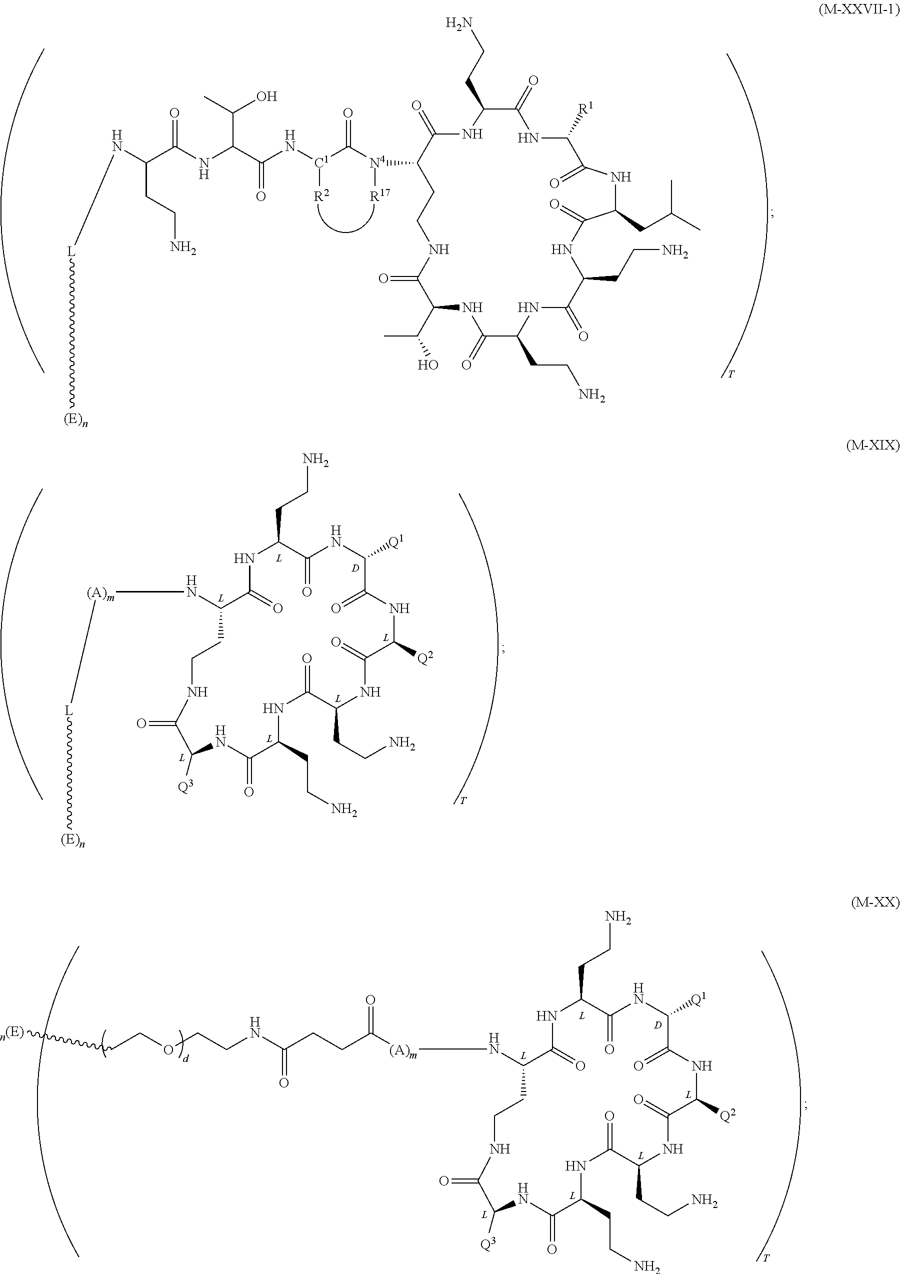

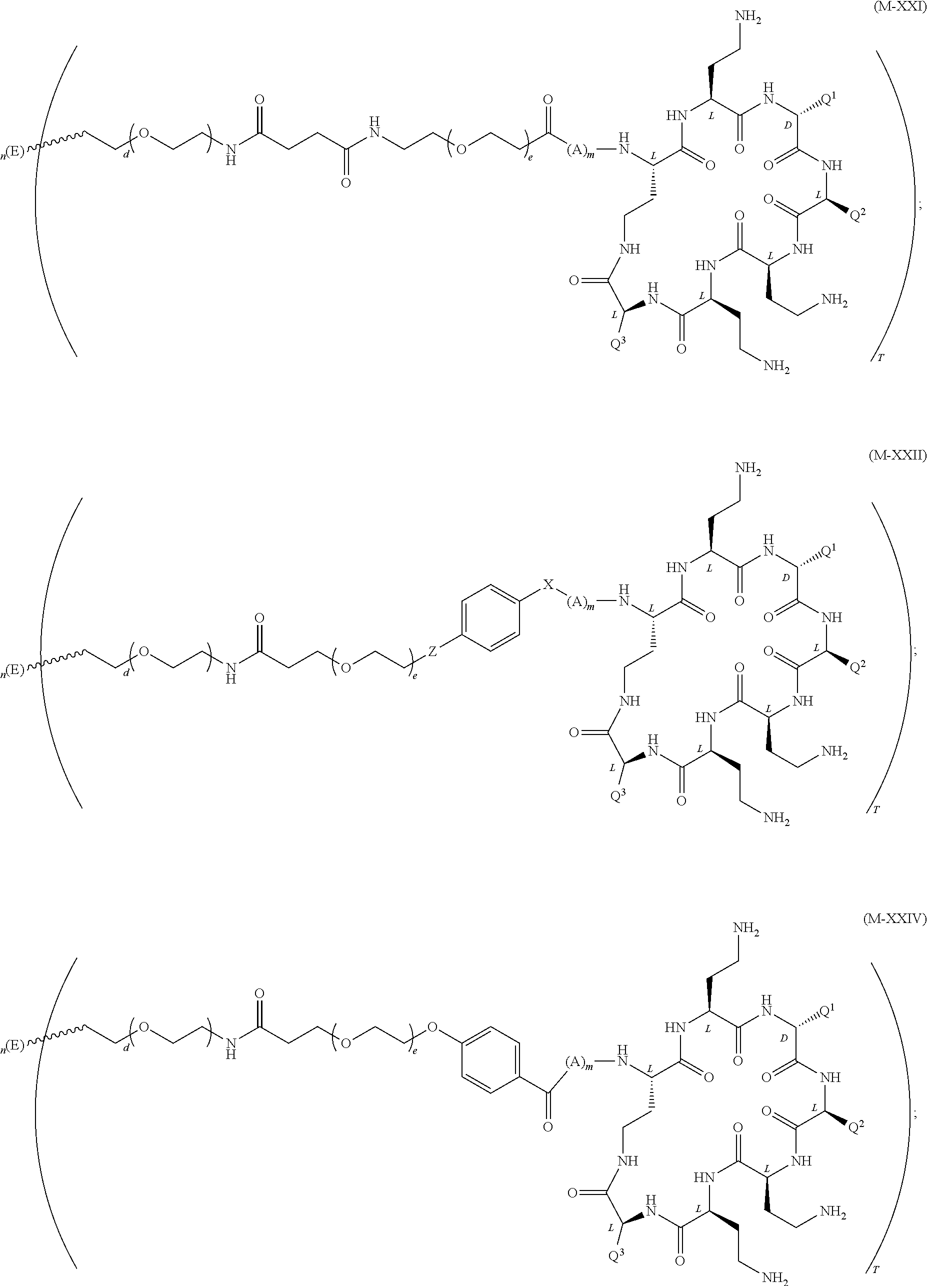

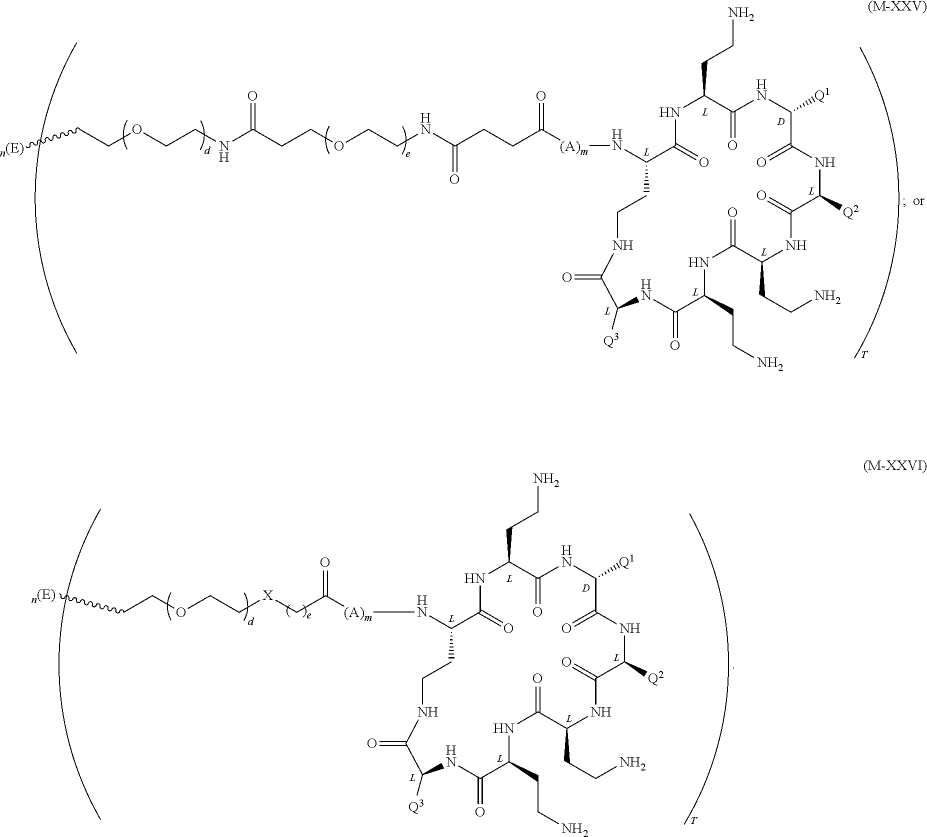

In conjugates having an Fc domain covalently linked to one or more monomers of cyclic heptapeptides, as represented by the formulae above, when n is 2, two Fc domain monomers (each Fc domain monomer is represented by E) dimerize to form an Fc domain.

### III. Fc Domain Monomers and Fc Domains

An Fc domain monomer includes a hinge domain, a C_{H}2 antibody constant domain, and a C_{H}3 antibody constant domain. The Fc domain monomer can be of immunoglobulin antibody isotype IgG, IgE, IgM, IgA, or IgD. The Fc domain monomer can also be of any immunoglobulin antibody isotype (e.g., IgG1, IgG2a, IgG2b, IgG3, or IgG4). The Fc domain monomer can also be of any species, e.g., human, murine, or mouse. A dimer of Fc domain monomers is an Fc domain that can bind to an Fc receptor, which is a receptor located on the surface of leukocytes.

In some embodiments, an Fc domain monomer in the conjugates described herein may contain one or more amino acid substitutions, additions, and/or deletion relative to an Fc domain monomer having a sequence of any one of SEQ ID NOs: 1-14, 1-29, or 1-31. In some embodiments, an Asn in an Fc domain monomer in the conjugates as described herein may be replaced by Ala in order to prevent N-linked glycosylation (see, e.g., SEQ ID NOs: 11-14, where Asn to Ala substitution is labeled with *). In some embodiments, an Fc domain monomer in the conjugates described herein may also containing additional Cys additions (see, e.g., SEQ ID NOs: 9 and 10, where Cys additions are labeled with *).

In some embodiments, an Fc domain monomer in the conjugates as described herein includes an additional moiety, e.g., an albumin-binding peptide, a purification peptide (e.g., a hexa-histidine peptide (HHHHHH (SEQ ID NO: 32)), or a signal sequence (e.g., IL2 signal sequence MYRMQLLSCIALSLALVTNS (SEQ ID NO: 33)) attached to the N- or C-terminus of the Fc domain monomer. In some embodiments, an Fc domain monomer in the conjugate does not contain any type of antibody variable region, e.g., V_{H}, V_{L}, a complementarity determining region (CDR), or a hypervariable region (HVR).

In some embodiments, an Fc domain monomer in the conjugates as described herein may have a sequence that is at least 95% identical (e.g., 97%, 99%, or 99.5% identical) to the sequence of any one of SEQ ID NOs: 1-14, 1-29, or 1-31 shown below. In some embodiments, an Fc domain monomer in the conjugates as described herein may have a sequence of any one of SEQ ID NOs: 1-14, 1-29, or 1-31 shown below.

SEQ ID NO: 1: murine Fc-IgG2a with IL2 signal sequence at the N-terminus (bold)

MYRMQLLSCIALSLALVTNSPRGPTIKPCPPCKCPAPNLLGGPSVFIFP
PKIKDVLMISLSPIVTCVVVDVSEDDPDVQISWFVNNVEVHTAQTQTHR
EDYNSTLRVVSALPIQHQDWMSGKEFKCKVNNKDLPAPIERTISKPKGS
VRAPQVYVLPPPEEEMTKKQVTLTCMVTDFMPEDIYVEWTNNGKTELNY
KNTEPVLDSDGSYFMYSKLRVEKKNWVERNSYSCSVVHEGLHNHHTTKS
FSRTPGK

SEQ ID NO: 2: mature murine Fc-IgG2a

PRGPTIKPCPPCKCPAPNLLGGPSVFIFPPKIKDVLMISLSPIVTCVVV
DVSEDDPDVQISWFVNNVEVHTAQTQTHREDYNSTLRVVSALPIQHQDW
MSGKEFKCKVNNKDLPAPIERTISKPKGSVRAPQVYVLPPPEEEMTKKQ
VTLTCMVTDFMPEDIYVEWTNNGKTELNYKNTEPVLDSDGSYFMYSKLR
VEKKNWVERNSYSCSVVHEGLHNHHTTKSFSRTPGK

SEQ ID NO: 3: human Fc-IgG1 with IL2 signal sequence at the N-terminus (bold)

(SEQ ID NO: 3)
MYRMQLLSCIALSLALVTNSMVRSDKTHTCPPCPAPELLGGPSVFLFPP
KPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSL
SLSPGK

SEQ ID NO: 4: mature human Fc-IgG1

MVRSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWL
NGKEYKCKVSNKALPAPIEKTISKAKGQPREPQVYTLPPSREEMTKNQV
SLTCLVKGFYPSDIAVEWESNGQPENNYKTTPPVLDSDGSFFLYSKLTV
DKSRWQQGNVFSCSVMHEALHNHYTQKSLSLSPGK

SEQ ID NO: 5: murine Fc-IgG2a with IL2 signal sequence (bold) at the N-terminus and hexa-histidine peptide (italicized) at the C-terminus

MYRMQLLSCIALSLALVTNSPRGPTIKPCPPCKCPAPNLLGGPSVFIFP
PKIKDVLMISLSPIVTCVVVDVSEDDPDVQISWFVNNVEVHTAQTQTHR
EDYNSTLRVVSALPIQHQDWMSGKEFKCKVNNKDLPAPIERTISKPKGS
VRAPQVYVLPPPEEEMTKKQVTLTCMVTDFMPEDIYVEWTNNGKTELNY
KNTEPVLDSDGSYFMYSKLRVEKKNWVERNSYSCSVVHEGLHNHHTTKS
FSRTPGKHHHHHH

SEQ ID NO: 6: mature murine Fc-IgG2a with hexa-histidine peptide (italicized) at the C-terminus

PRGPTIKPCPPCKCPAPNLLGGPSVFIFPPKIKDVLMISLSPIVTCVVV
DVSEDDPDVQISWFVNNVEVHTAQTQTHREDYNSTLRVVSALPIQHQDW
MSGKEFKCKVNNKDLPAPIERTISKPKGSVRAPQVYVLPPPEEEMTKKQ
VTLTCMVTDFMPEDIYVEWTNNGKTELNYKNTEPVLDSDGSYFMYSKLR
VEKKNWVERNSYSCSVVHEGLHNHHTTKSFSRTPGKHHHHHH

SEQ ID NO: 7: human Fc-IgG1 with IL2 signal sequence (bold) at the N-terminus and hexa-histidine peptide (italicized) at the C-terminus

MYRMQLLSCIALSLALVTNSMVRSDKTHTCPPCPAPELLG
GPSVFLFPPKPKDTLMISRTPEVTCVVVDVSHEDPEVKFNW
YVDGVEVHNAKTKPREEQYNSTYRVVSVLTVLHQDWLNGKE
YKCKVSNKALPAPIEKTISKAKGQPREPQVYTLPPSREEM
TKNQVSLTCLVKGFYPSDIAVEWESNGQPENNYKTTPPVL
DSDGSFFLYSKLTVDKSRWQQGNVFSCSVMHEALHNHYTQ
KSLSLSPGKHHHHHH

SEQ ID NO: 8: mature human Fc-IgG1 with hexa-histidine peptide (italicized) at the C-terminus

MVRSDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNG
KEYKCKVSNKALPAPIEKTISKAKGQPREPQVYTLPPSREEMTKNQVSLT
CLVKGFYPSDIAVEWESNGQPENNYKTTPPVLDSDGSFFLYSKLTVDKSR
WQQGNVFSCSVMHEALHNHYTQKSLSLSPGKHHHHHH

SEQ ID NO: 9: human Fc-IgG1 with IL2 signal sequence (bold) at the N-terminus, two additional cysteines in the hinge region (*), and hexa-histidine peptide (italicized) at the C-terminus

MYRMQLLSCIALSLALVTNSMVRSDKTHTCPPCPPC*KC*PAPELLGGPS
VFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQK
SLSLSPGKHHHHHH

SEQ ID NO: 10: mature human Fc-IgG1 with two additional cysteines in the hinge region (*) and hexa-histidine peptide (italicized) at the C-terminus

MVRSDKTHTCPPCPPC*KC*PAPELLGGPSVFLFPPKPKDTLMISRTPEV
TCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSREEMT
KNQVSLTCLVKGFYPSDIAVEWESNGQPENNYKTTPPVLDSDGSFFLYSK
LTVDKSRWQQGNVFSCSVMHEALHNHYTQKSLSLSPGKHHHHHH

SEQ ID NO: 11: murine Fc-IgG2a with IL2 signal sequence (bold) at the N-terminus, Asn to Ala substitution (*), and hexa-histidine peptide (italicized) at the C-terminus

MYRMQLLSCIALSLALVTNSPRGPTIKPCPPCKCPAPNLLGGPSVFIFPP
KIKDVLMISLSPIVTCVVVDVSEDDPDVQISWFVNNVEVHTAQTQTHRED
YA*STLRVVSALPIQHQDWMSGKEFKCKVNNKDLPAPIERTISKPKGSVR
APQVYVLPPPEEEMTKKQVTLTCMVTDFMPEDIYVEWTNNGKTELNYKNT
EPVLDSDGSYFMYSKLRVEKKNWVERNSYSCSVVHEGLHNHHTTKSFSRT
PGKHHHHHH

SEQ ID NO: 12: mature murine Fc-IgG2a with Asn to Ala substitution (*) and hexa-histidine peptide (italicized) at the C-terminus

PRGPTIKPCPPCKCPAPNLLGGPSVFIFPPKIKDVLMISLSPIVTCVVVV
DVSEDDPDVQISWFVNNVEVHTAQTQTHREDYA*STLRVVSALPIQHQDW
MSGKEFKCKVNNKDLPAPIERTISKPKGSVRAPQVYVLPPPEEEMTKKQV
TLTCMVTDFMPEDIYVEWTNNGKTELNYKNTEPVLDSDGSYFMYSKLRVE
KKNWVERNSYSCSVVHEGLHNHHTTKSFSRTPGKHHHHHH

SEQ ID NO: 13: human Fc-IgG1 with IL2 signal sequence (bold) at the N-terminus, Asn to Ala substitution (*), and hexa-histidine peptide (italicized) at the C-terminus

MYRMQLLSCIALSLALVTNSMVRSDKTHTCPPCPAPELLGGPSVFLFPPK
PKDTLMISRTPEVTCVVVDVSHEDPEVKFNWYVDGVEVHNAKTKPREEQY
A*STYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GKHHHHHH

SEQ ID NO: 14: mature human Fc-IgG1 with Msn to Ala substitution (*) and hexa-histidine peptide (italicized) at the C-terminus

MVRSDKTHTCPPCPAPELLGGPSVFLFPPKPKDTLMISRTPEVTCVVVDV
SHEDPEVKFNWYVDGVEVHNAKTKPREEQYA*STYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGVNFSCSVMHEALHNHYTQKSLSLSPGKHHHHHH

SEQ ID NO: 15: human IgG1 Fc with Human Serum Albumin Signal Sequence (bold) at the N-terminus

MKWVTFISLLFLFSSAYSISAMVRSDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK

SEQ ID NO: 16: human IgG1 Fc with Human Serum Albumin Signal Sequence (bold) at the N-terminus then terminal K removed, G4S linker (italicized) and then c-Myc tag

MKWVTFISLLFLFSSAYSISAMVRSDKTHTCPPVPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GGGGSEQKLISEEDL

SEQ ID NO: 17: mature human IgG1 Fc with terminal K removed, G4S linker (italicized) and then c-Myc tag

ISAMVRSDKTHTCPPCPAPELLGGPSVFLFPPKPKDTLMISRTEPVTCVV
VDVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQV
SLTCLVKGFYPSDIAVEWESNGQPENNKYTTPPVLDSDGSFFLYSKLTVD
KSRWQQGNVFSCSVMHEALHNHYTQKSLSLSPGGGGSEQKLISEEDL

SEQ ID NO: 18: human IgG1 Fc with Human Serum Albumin Signal Sequence (bold), with lysine to serine modification (*) to prevent lysine conjugation at this site

MKWVTFISLLFLFSSAYSISAMVRSDKTHTCPPCPAPELLGGPSVFLFPP
KPS*DTLMISRTPEVTCVVVDVSHEDPEVKFNWYVDGVEVHNAKTKPREE
QYNSTYRVVSVLTVLHQDWLNGKEYKCKVSNKALPAPEIKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLS
PGK

SEQ ID NO: 19: mature human IgG1 Fc with lysine to serine modification (*) to prevent lysine conjugation at this site

ISAMVRSDKTHTCPPCPAPELLGGPSVFLFPPKPS*DTLMISRTPEVTCV
VVDVSHEDPEVKFNWYVGDVEVHNAKTKPREEQYNSTYRVVSVLTVLHQD
WLNGKEYKCKVSNKALPAPIEKTISKAKGQPREPQVYTLPSREEMTKNQV
SLTCLVKGFYPSDIAVEWESNGQPENNYKTTPPVLDSDGSFFLYSKLTVD
KSRWQQGNVFSCSVMHEALHNHYTQKSLSLSPGK

SEQ ID NO: 20: human IgG1 Fc with Human Serum Albumin Signal Sequence (bold) at the N-terminus with lysine to serine modification (*) to prevent lysine conjugation at this site, G4S linker (italicized) and C-Myc tag

MKWVTFISLLFLFSSAYSISAMVRSDKTHTCPPCPAPELLGGPSVFLFPP
KPS(*)DTLMISRTPEVTCVVVDVSHEDPEVFKNWYVDGVEVHNAKTKPR
EEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYK
TTPVLDSDGSFFLYSKLTVDKSRWQQGNVFSCSVMHEALHNHYTQKSLSL
SPGGGGGSEQKLISEEDL

SEQ ID NO: 21: mature human IgG1 Fc with lysine to serine modification (*) to prevent lysine conjugation at this site, G4S linker (italicized) and C-Myc tag

ISAMVRSDKTHTCPPCPAPELLGGPSVFLFPPKPS(*)DTLMISRTPEV
TCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFL
YSKLTVDKSRWQQGNVFSCSVMHEALHNHYTQKSLSLSPGGGGGSEQKL
ISEEDL

SEQ ID NO: 22: human IgG1 Fc with Human Serum Albumin Signal Sequence (bold) at the N-terminus, Asn to Ala substitution (*), G4S linker (italicized) and C-myc tag

MKWVTFISLLFLFSSAYSISAMVRSDKTHTCPPCPAPELLGGPSVFLFPP
KPKDTLMISRTPEVTCVVVDVSHEDPEVKFNWYVDGVEVHNAKTKPREEQ
YA(*)STYRVVSVLTVLHQDWLNGKEYKCKVSNKALPAPIEKTISKAKGQ
PREPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSLS
LSPGGGGGSEQKLISEEDL

SEQ ID NO: 23: mature human IgG1 Fc with Msn to Ala substitution (*), G4S linker (italicized) and C-myc tag

ISAMVRSDKTHTCPPCPAPELLGGPSVFLFPPKPKDTLMISRTPEVTCV
VVDVSHEDPEVKFNWYVDGVEVHNAKTKPREEQYA(*)STYRVVSVLTV
LHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFL
YSKLTVDKSRWQQGNVFSCSVMHEALHNHYTQKSLSLSPGGGGGSEQKL
ISEEDL

SEQ ID NO: 24: human IgG1 Fc with Human Serum Albumin Signal Sequence (bold) at the N-terminus, with H310A (*) and H435A (*) mutations to impede FcRn binding G4S (italicized) and C-myc tag

MKWVTFISLLFLFSSAYSISAMVRSDKTHTCPPCPAPELLGGPSVFLFP
PKPKDTLMISRTPEVTCVVVDVSHEDPEVKFNWYVDGVEVHNAKTKPRE
EQYNSTYRVVSVLTVLA(*)QDWLNGKEYKCKVSNKALPAPIEKTISKA
(*)KGQPREPQVYTLPPSREEMTKNQVSLTCLVKGFYPSDIAVEWESNG
QPENNYKTTPPVLDSDGSFFLYSKLTVDKSRWQQGNVFSCSVMHEALHN
AYTQKSLSLSPGGGGGSEQKLISEEDL

SEQ ID NO: 25: mature human IgG1 Fc with Human Serum Albumin Signal Sequence (bold) at the N-terminus, with H310A (*) and H435A (*) mutations to impede FcRn binding G4S (italicized) and C-myc tag

ISAMVRSDKTHTCPPCPAPELLGGPSVFLFPPKPKDTLMISRTPEVTCV
VVDVSHEDPEVKFNWYVDGVEVHNAKTKPREEQYNSTYRVVSVLTVLA
(*)QDWLNGKEYKCKVSNKALPAPIEKTISKA(*)KGQPREPQVYTLPP
SREEMTKNQVSLTCLVKGFYPSDIAVEWESNGQPENNYKTTPPVLDSDG
SFFLYSKLTVDKSRWQQGNVFSCSVMHEALHNAYTQKSLSLSPGGGGGS
EQKLISEEDL

SEQ ID NO: 26: human IgG1 Fc with Human Serum Albumin Signal Sequence (bold) at the N-terminus, G4S linker (italicized) and mutated (lysine to phenylalanine, bold) C-myc tag

MKWVTFISLLFLFSSAYSISAMVRSDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKS
LSLSPGGGGGSEQFLISEEDL

SEQ ID NO: 27: mature human IgG1 Fc with G4S linker (italicized) and mutated (lysine to phenylalanine, bold) C-myc tag

ISAMVRSDKTHTCPPCPAPELLGGPSVFLFPPKPKDTLMISRTPEVTCV
VVDVSHEDPEVKFNWYVDGVEVHNAKTKPREEQYNSTYRVVSVLTVLHQ
DWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSK
LTVDKSRWQQGNVFSCSVMHEALHNHYTQKSLSLSPGGGGGSEQFLISE
EDL

SEQ ID NO: 28: human IgG1 Fc with Human Serum Albumin Signal Sequence (bold) at the N-terminus, Asn to Ala substitution (*), G4S linker (italicized) and mutated (lysine to phenylalanine, bold) C-myc tag

MKWVTFISLLFLFSSAYSISAMVRSDKTHTCPPCPAPELLGGPSVFLFP
PKPKDTLMISRTPEVTCVVVDVSHEDPEVKFNWYVDGVEVHNAKTKPRE
EQYA(*)STYRVVSVLTVLHQDWLNGKEYKCKVSNKALPAPIEKTISKA
KGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYT
QKSLSLSPGGGGGSEQFLISEEDL

SEQ ID NO: 29: mature human IgG1 Fc with Asn to Ala substitution (*), G4S linker (italicized) and mutated (lysine to phenylalanine, bold) C-myc tag

ISAMVRSDKTHTCPPCPAPELLGGPSVFLFPPKPKDTLMISRTPEVTCV
VVDVSHEDPEVKFNWYVDGVEVHNAKTKPREEQYAQSTYRVVSVLTVLH
QDWLNGKEYKCKVSNKALPAPIEKTISKAKGQPREPQVYTLPPSREEMT
KNQVSLTCLVKGFYPSDIAVEWESNGQPENNYKTTPPVLDSDGSFFLYS
KLTVDKSRWQQGNVFSCSVMHEALHNHYTQKSLSLSPGGGGGSEQFLIS
EEDL

SEQ ID NO: 30: human IgG1 Fc with Human Serum Albumin Signal Sequence (bold) at the N-terminus, removal of the residues ISAMVRS from the N-terminus of the mature human IgG1 Fc, Glu to Asp substitution (*), Met to Leu substitution (**), G4S linker (italicized), and mutated (lysine to phenylalanine, bold) C-myc tag

MKWVTFISLLFLFSSAYSDKTHTCPPCPAPELLGGPSVFLFPPKPKDTL
MISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVY
TLPPSRD(*)EL(**)TKNQVSLTCLVKGFYPSDIAVEWESNGQPENNY
KTTPPVLDSDGSFFLYSKLTVDKSRWQQGNVFSCSVMHEALHNHYTQKS
LSLSPGGGGGSEQFLISEEDL

SEQ ID NO: 31: human IgG1 Fc with Human Serum Albumin Signal Sequence (bold) at the N-terminus, removal of the residues ISAMVRS from the N-terminus of the mature human IgG1 Fc, Glu to Asp substitution (*), and Met to Leu substitution (**)

MKWVTFISLLFLFSSAYSDKTHTCPPCPAPELLGGPSVFLFPPKPKDTL
MISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVY
TLPPSRD(*)EL(**)TKNQVSLTCLVKGFYPSDIAVEWESNGQPENNY
KTTPPVLDSDGSFFLYSKLTVDKSRWQQGNVFSCSVMHEALHNHYTQKS
LSLSPGK

As defined herein, an Fc domain includes two Fc domain monomers that are dimerized by the interaction between the CH₃ antibody constant domains, as well as one or more disulfide bonds that form between the hinge domains of the two dimerizing Fc domain monomers. An Fc domain forms the minimum structure that binds to an Fc receptor, e.g., Fc-gamma receptors (i.e., Fcγ receptors (FcγR)), Fc-alpha receptors (i.e., Fcα receptors (FcαR)), Fc-epsilon receptors (i.e., Fcε receptors (FcεR)), and/or the neonatal Fc receptor (FcRn). In some embodiments, an Fc domain of the present invention binds to an Fcγ receptor (e.g., FcRn, FcγRI (CD64), FcγRIIa (CD32), FcγRIIb (CD32), FcγRIIIa (CD16a), FcγRIIIb (CD16b)), and/or FcγRIV and/or the neonatal Fc receptor (FcRn).

In some embodiments, the Fc domain monomer or Fc domain of the invention is an aglycosylated Fc domain monomer or Fc domain (e.g., an Fc domain monomer or and Fc domain that maintains engagement to an Fc receptor (e.g., FcRn). For example, the Fc domain is an aglycosylated IgG1 variants that maintains engagement to an Fc receptor (e.g., an IgG1 having an amino acid substitution at N297 and/or T299 of the glycosylation motif). Exemplary aglycosylated Fc domains and methods for making aglycosylated Fc domains are known in the art, for example, as described in Sazinsky S. L. et al., Aglycosylated immunoglobulin G1 variants productively engage activating Fc receptors, PNAS, 2008, 105(51):20167-20172, which is incorporated herein in its entirety.

In some embodiments, the Fc domain or Fc domain monomer of the invention is engineered to enhance binding to the neonatal Fc receptor (FcRn). For example, the Fc domain may include the triple mutation M252Y/S254T/T256E (YTE) (e.g., an IgG1, such as a human or humanized IgG1 having a YTE mutation). The Fc domain may include the double mutant M428L/N434S (LS) (e.g., an IgG1, such as a human or humanized IgG1 having an LS mutation). The Fc domain may include the single mutant N434H (e.g., an IgG1, such as a human or humanized IgG1 having an N434H mutation). The Fc domain may include a combination of one or more of the above-described mutations that enhance binding to the FcRn. Exemplary Fc domains with enhanced binding to the FcRN and methods for making Fc domains having enhanced binding to the FcRN are known in the art, for example, as described in Maeda, A. et al., MABS, 2017, 9(5):844-853, which is incorporated herein in its entirety.

As used herein, a sulfur atom “corresponding to” a particular cysteine residue of a particular SEQ ID NO. should be understood to include the sulfur atom of any cysteine residue that one of skill in the art would understand to align to the particular cysteine of the particular sequence. The protein sequence alignment of human IgG1 (UniProtKB: P01857), human IgG2 (UniProtKB: P01859), human IgG3 (UniProtKB: P01860), and human IgG4 (UniProtKB: P01861) is provided below (aligned with Clustal Omega Multiple Pairwise Alignment). The alignment indicates cysteine residues (e.g., sulfur atoms of cysteine residues) that “correspond to” one another (in boxes and indicated by the • symbol). One of skill in the art would readily be able to perform such an alignment with any IgG variant of the invention to determine the sulfur atom of a cysteine that corresponds to any sulfur atom of a particular cysteine of a particular SEQ ID NO described herein. For example, one of skill in the art would readily be able to determine that Cys10 of SEQ ID NO: 10 (the first cysteine of the conserved CPPC motif of the hinge region of the Fc domain) corresponds to, for example, Cys109 of IgG1, Cys106 of IgG2, Cys156 of IgG3, Cys29 of SEQ ID NO: 1, Cys9 of SEQ ID NO: 2, Cys30 of SEQ ID NO: 3, or Cys10 of SEQ ID NO: 10.

As used herein, a nitrogen atom “corresponding to” a particular lysine residue of a particular SEQ ID NO. should be understood to include the nitrogen atom of any lysine residue that one of skill in the art would understand to align to the particular lysine of the particular sequence. The protein sequence alignment of human IgG1 (UniProtKB: P01857), human IgG2 (UniProtKB: P01859), human IgG3 (UniProtKB: P01860), and human IgG4 (UniProtKB: P01861) is provided below (aligned with Clustal Omega Multiple Pairwise Alignment). The alignment indicates lysine residues (e.g., nitrogen atoms of lysine residues) that “correspond to” one another (in boxes and indicated by the * symbol). One of skill in the art would readily be able to perform such an alignment with any IgG variant of the invention to determine the nitrogen atom of a lysine that corresponds to any nitrogen atom of a particular lysine of a particular SEQ ID NO described herein. For example, one of skill in the art would readily be able to determine that Lys35 of SEQ ID NO: 10 corresponds to, for example, Lys129 of IgG1, Lys126 of IgG2, Lys176 of IgG3, Lys51 of SEQ ID NO: 1, Lys31 of SEQ ID NO: 2, Lys50 of SEQ ID NO: 3, or Lys30 of SEQ ID NO: 10.

### Protein Sequence Alignment of IgG1, IgG2, IgG3, and IgG4

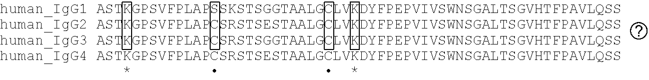

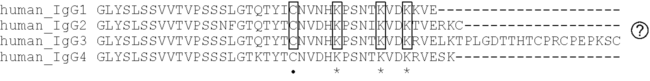

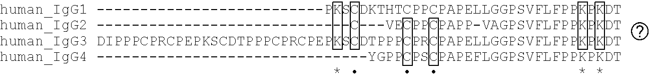

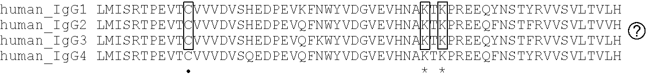

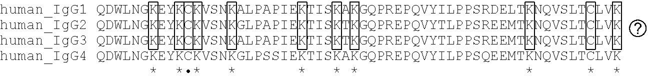

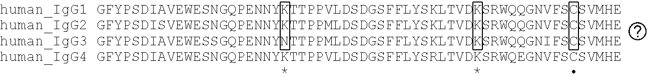

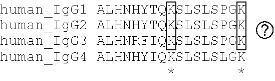

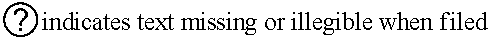

Activation of Immune Cells

Fcgamma receptors (FcγRs) bind the Fc portion of immunoglobulin G (IgG) and play important roles in immune activation and regulation. For example, the IgG Fc domains in immune complexes (ICs) engage FcγRs with high avidity, thus triggering signaling cascades that regulate immune cell activation. The human FcγR family contains several activating receptors (FcγRI, FcγRIIa, FcγRIIc, FcγRIIIa, and FcγRIIIb) and one inhibitory receptor (FcγRIIb). FcγR signaling is mediated by intracellular domains that contain immune tyrosine activating motifs (ITAMs) for activating FcγRs and immune tyrosine inhibitory motifs (ITIM) for inhibitory receptor FcγRIIb. In some embodiments, FcγR binding by Fc domains results in ITAM phosphorylation by Src family kinases; this activates Syk family kinases and induces downstream signaling networks, which include PI3K and Ras pathways.

In the conjugates described herein, the portion of the conjugates including monomers or dimers of cyclic heptapeptides bind to lipopolysaccharides (LPS) in the cell membrane of Gram-negative bacteria to disrupt and permeabilize the cell membrane, leading to cell death and/or sensitization to other antibiotics, while the Fc domain portion of the conjugates bind to FcγRs (e.g., FcRn, FcγRI, FcγRIIa, FcγRIIc, FcγRIIIa, and FcγRIIIb) on immune cells and activate phagocytosis and effector functions, such as antibody-dependent cell-mediated cytotoxicity (ADCC), thus leading to the engulfment and destruction of bacterial cells by immune cells and further enhancing the antibacterial activity of the conjugates. Examples of immune cells that may be activated by the conjugates described herein include, but are not limited to, macrophages, neutrophils, eosinophils, basophils, lymphocytes, follicular dendritic cells, natural killer cells, and mast cells.

### IV. Linkers

A linker refers to a linkage or connection between two or more components in a conjugate described herein (e.g., between two cyclic heptapeptides in a conjugate described herein, between a cyclic heptapeptide and an Fc domain in a conjugate described herein, and between a dimer of two cyclic peptapeptide and an Fc domain in a conjugate described herein).

Linkers in Conjugates Having an Fc Domain Covalently Linked to Dimers of Cyclic Heptapeptides

In a conjugate containing an Fc domain covalently linked to one or more dimers of cyclic heptapeptides as described herein, a linker in the conjugate (e.g., L′, L, or L¹) may be a branched structure. As described further herein, a linker in a conjugate described herein (e.g., L′, L, or L¹) may be a multivalent structure, e.g., a divalent or trivalent structure having two or three arms, respectively. In some embodiments when the linker has three arms, two of the arms may be attached to the first and second cyclic heptapeptides and the third arm may be attached to the Fc domain monomer or Fc domain. In some embodiments when the linker has two arms, one arm may be attached to an Fc domain and the other arm may be attached to one of the two cyclic heptapeptides. In other embodiments, a linker with two arms may be used to attach the two cyclic heptapeptides on a conjugate containing an Fc domain covalently linked to one or more dimers of cyclic heptapeptides.

In some embodiments, the linker L′ in the conjugate described by formula (I) is described by formula (L):

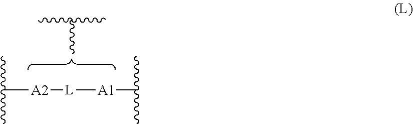

in which L is a remainder of L′; A1 is a 1-5 amino acid peptide (e.g., a 1-4, 1-3, or 1-2 amino acid peptide) covalently attached to the linking nitrogen in each M1 or is absent; and A2 is a 1-5 amino acid peptide (e.g., a 1-4, 1-3, or 1-2 amino acid peptide) covalently attached to the linking nitrogen in each M2 or is absent. In some embodiments, a conjugate described herein may contain a linker that has a trivalent structure (e.g., a trivalent linker; a linker of formula (L-1)). A trivalent linker has three arms, in which each arm is conjugated to a component of the conjugate (e.g., a first arm conjugated to a first cyclic heptapeptide, a second arm conjugated to a second heptapeptide, and a third arm conjugated to an Fc domain in the conjugate described herein). In some embodiments, the one or more dimers of cyclic heptapeptides in the conjugates described herein may each be, independently, connected to an atom in the Fc domain.

In some embodiments, a linker in a conjugate having an Fc domain covalently linked to one or more dimers of cyclic heptapeptides is described by formula (D-L-1):

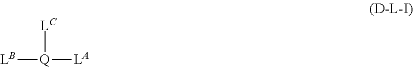

wherein L^{A} is described by formula G^{A1}-(Z^{A1})_{g1}-(Y^{A1})ₕ₁-(Z^{A2})ᵢ₁-(Y^{A2})ⱼ₁-(Z^{A3})ₖ₁-(Y^{A3})ₗ₁-(Z^{A4})ₘ₁-(Y^{A4})ₙ₁-(Z^{A5})_{O1}-G^{A2}; L^{B} is described by formula G^{B1}-(Z^{B1})_{g2}-(Y^{B1})ₕ₂-(Z^{B2})ᵢ₂-(Y^{B2})ⱼ₂-(Z^{B3})ₖ₂-(Z^{B3})ₗ₂-(Z^{B4})ₘ₂-(Y^{B4})ₙ₂-(Z^{B5})_{O2}-G^{B2}; L^{C} is described by formula G^{C1}-(Z^{C1})_{g3}-(Y^{C1})ₕ₃-(Z^{C2})ᵢ₃-(Y^{C2})ⱼ₃-(Z^{C3})ₖ₃-(Y^{C3})ₗ₃-(Z^{C4})ₘ₃-(Y^{C4})ₙ₃-(Z^{C5})_{O}_{₃}-G^{C2}; G^{A1} is a bond attached to Q in formula (D-L-I); G^{A2} is a bond attached to the first cyclic heptapeptide or a peptide (e.g., a peptide including 1-5 amino acid residue(s)) attached to the first cyclic heptapeptide, if the peptide is present; G^{B1} is a bond attached to Q in formula (D-L-1); G^{B2} is a bond attached to the second cyclic heptapeptide or a peptide (e.g., a peptide including 1-5 amino acid residue(s)) attached to the second cyclic heptapeptide, if the peptide is present; G^{C1} is a bond attached to Q in formula (D-L-I); G^{C2} is a bond attached to an Fc domain; each of Z^{A1}, Z^{A2}, Z^{A3}, Z^{A4}, Z^{A5}, Z^{B1}, Z^{B2}, Z^{B3}, Z^{B4}, Z^{B5}, Z^{C1}, Z^{C2}, Z^{C3}, Z^{C4}, and Z^{C5} is, independently, optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene; each of Y^{A1}, Y^{A2}, Y^{A3}, Y^{A4}, Y^{B1}, Y^{B2}, Y^{B3}, Y^{B4}, Y^{C1}, Y^{C2}, Y^{C3}, and Y^{C4} is, independently, O, S, NRⁱ, P, carbonyl, thiocarbonyl, sulfonyl, phosphate, phosphoryl, or imino; Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C3-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl; each of g1, h1, i1, j1, k1, l1, m1, n1, o1, g2, h2, i2, j2, k2, l2, m2, n2, o2, g3, h3, i3, j3, k3, l3, m3, n3, and o3 is, independently, 0 or 1; Q is a nitrogen atom, optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene.

In some embodiments, depending on the structure and chemical formula of L^{C}. L^{C} may have two points of attachment to the Fc domain (e.g., two G^{C2}).

Linkers of formula (D-L-1) that may be used in conjugates described herein include, but are not limited to,

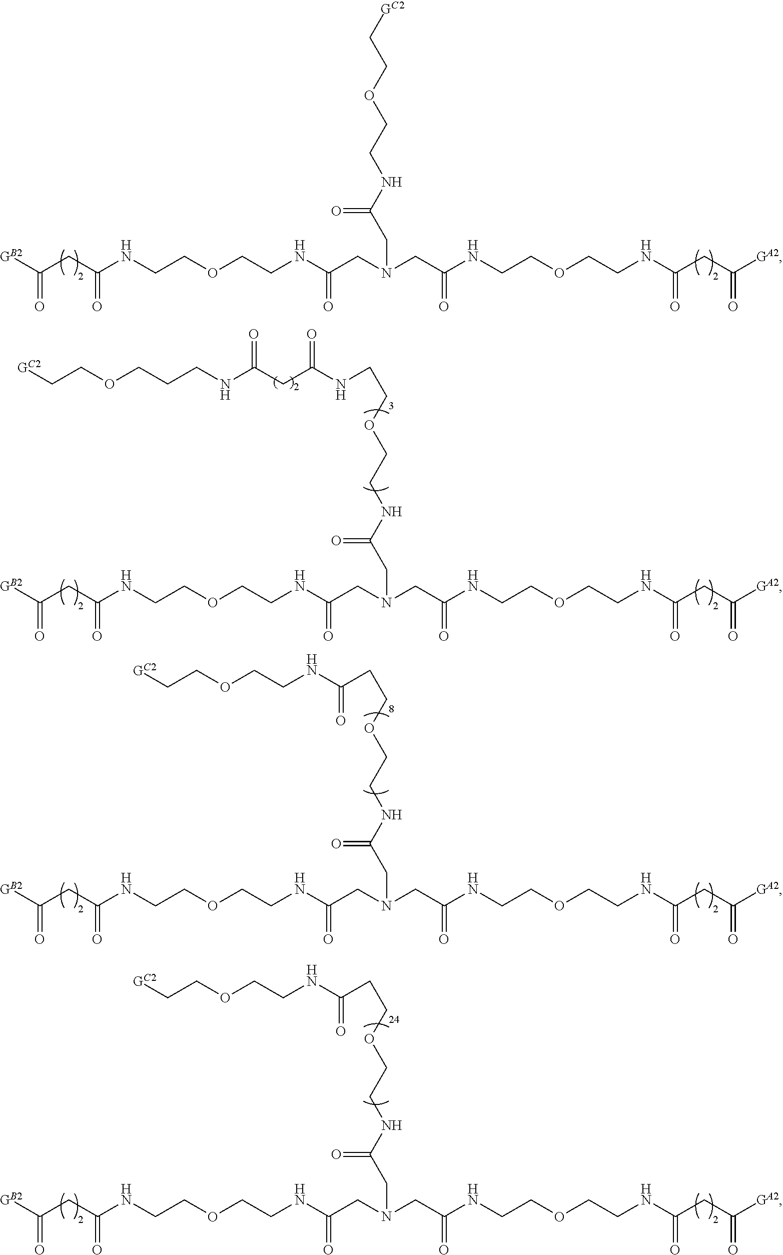

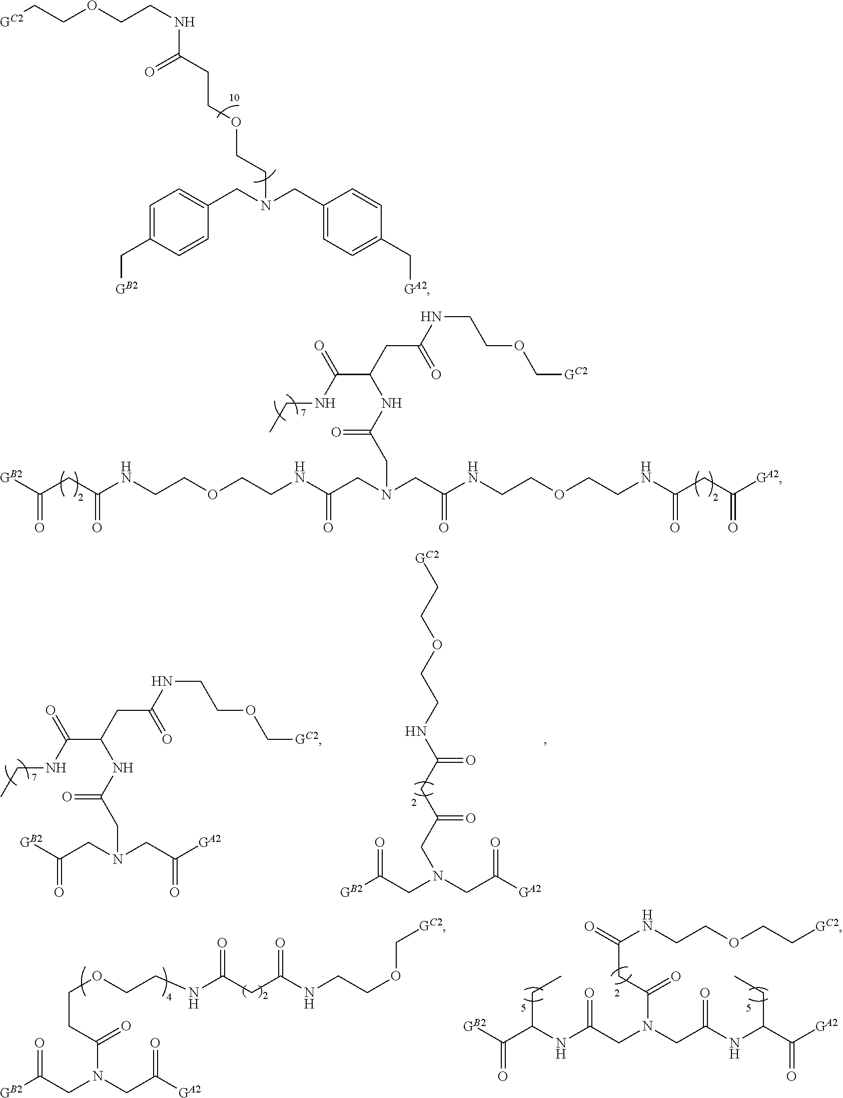

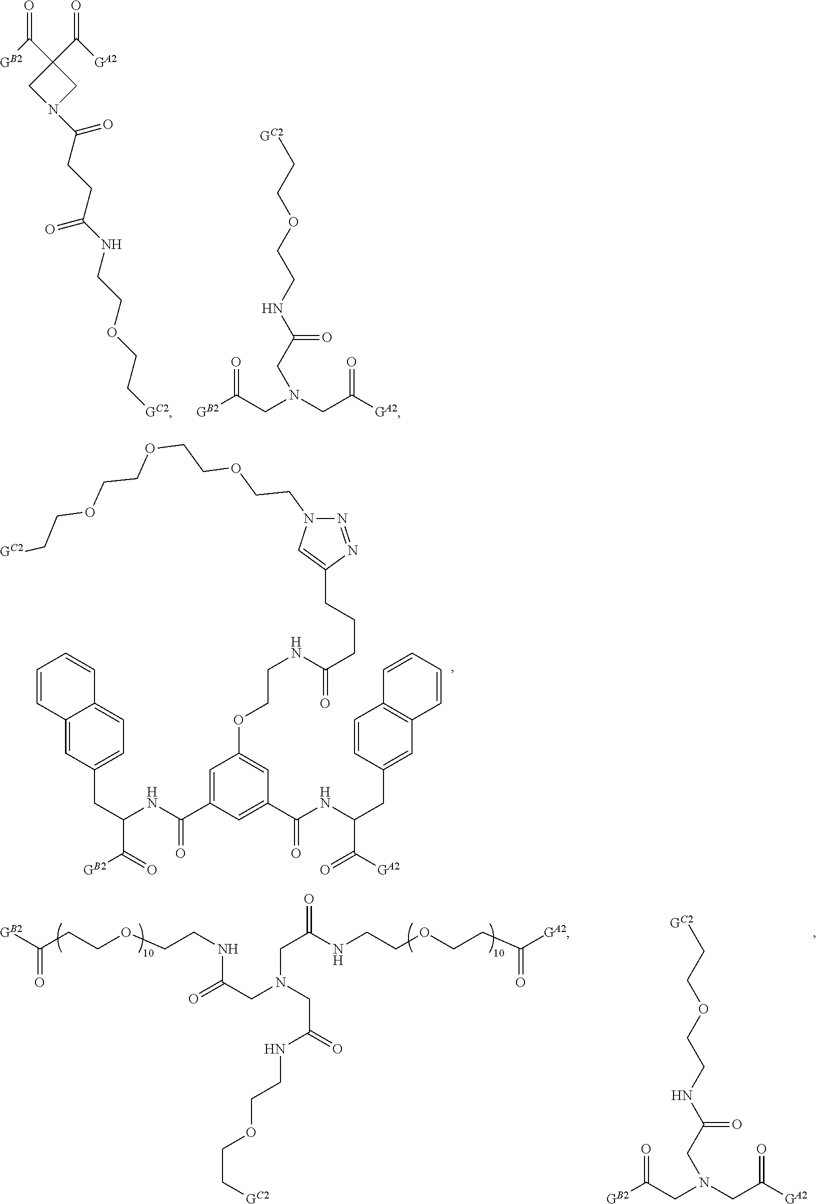

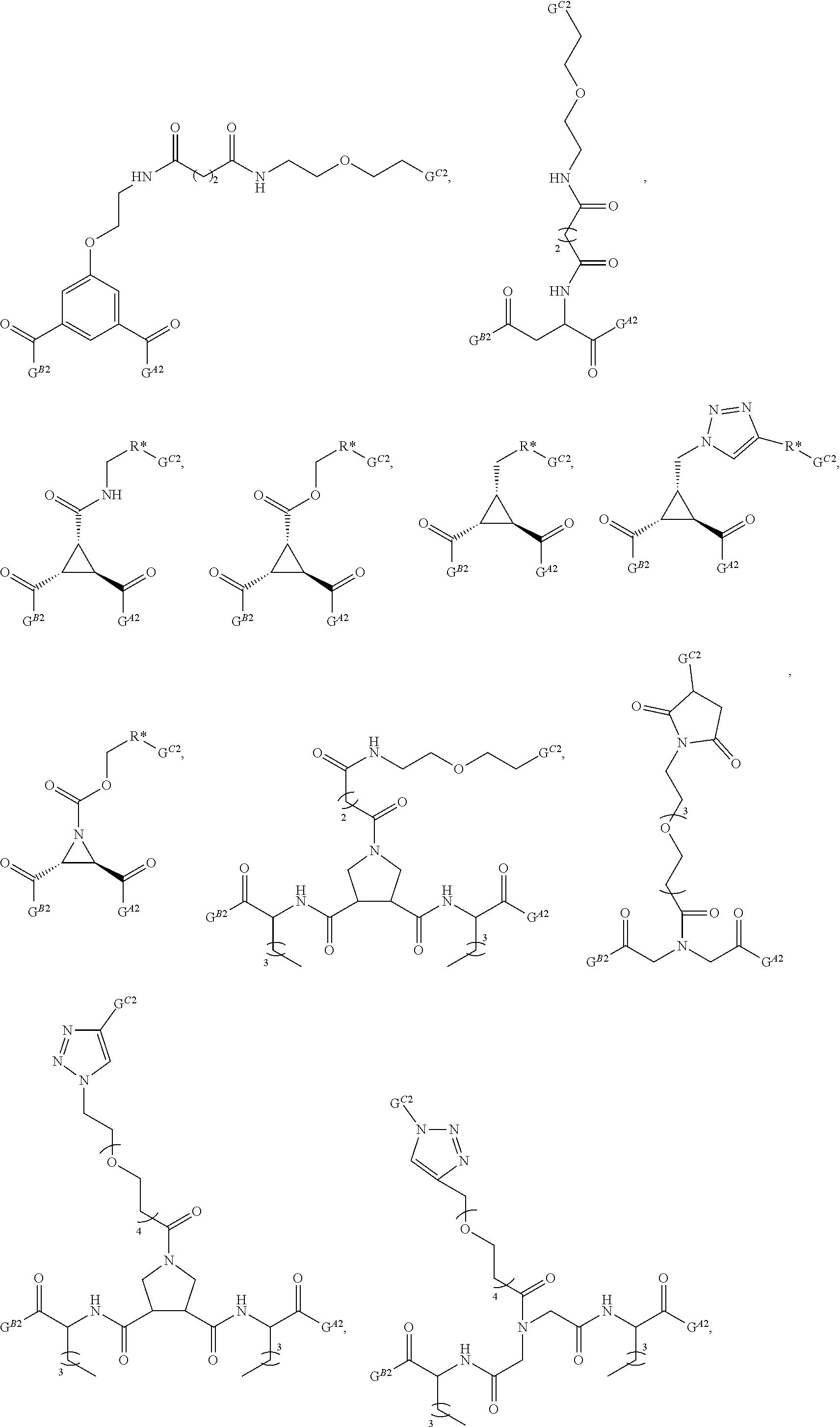

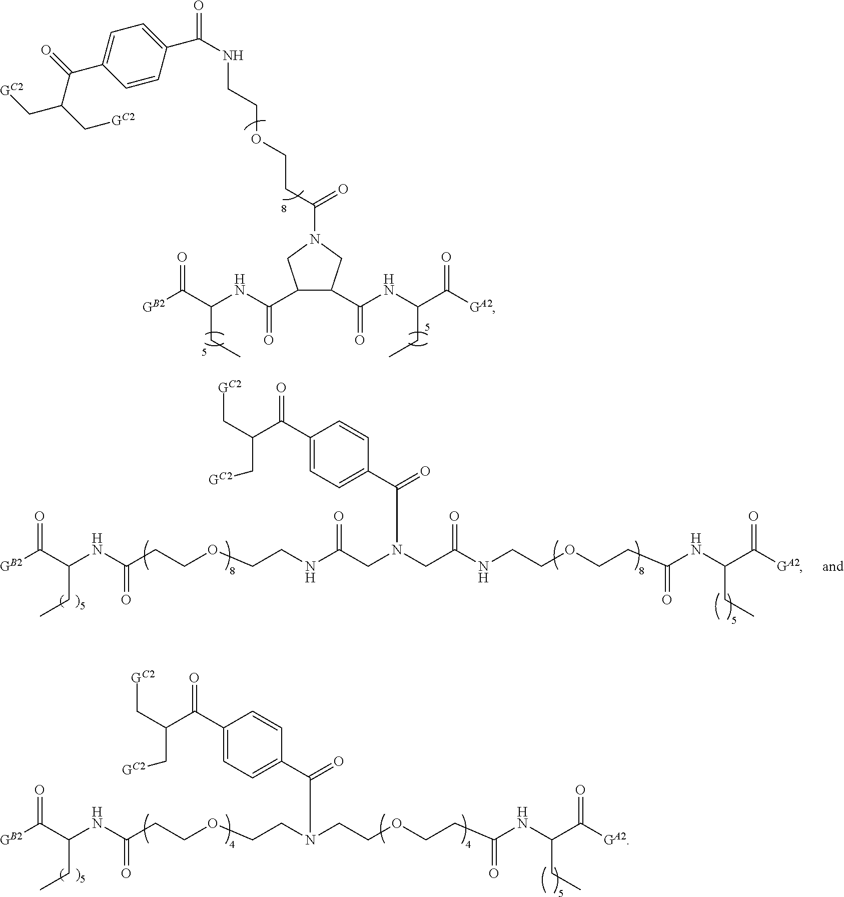

In some embodiments, a linker in a conjugate having an Fc domain covalently linked to one or more dimers of cyclic heptapeptides is described by formula (D-L-II):

L^{B}-Q-L^{A} (D-L-II)

wherein L^{A} is described by formula G^{A1}-(Z^{A1})_{g1}-(Y^{A1})ₕ₁-(Z^{A2})ᵢ₁-(Y^{A2})ⱼ₁-(Z^{A3})ₖ₁-(Y^{A3})ₗ₁-(Z^{A4})ₘ₁-(Y^{A4})ₙ₁-(Z^{A5})_{O}_{₁}-G^{A1}; L^{B} is described by formula G^{B1}-(Z^{B1})_{g2}-(Y^{B1})ₕ₂-(Z^{B2})ᵢ₂-(Y^{B2})ⱼ₂-(Z^{B3})ₖ₂-(Y^{B3})ₗ₂-(Z^{B4})ₘ₂-(Y^{B4})ₙ₂-(Z^{B5})ₒ₂-G^{B2}; G^{A1} is a bond attached to Q; G^{A2} is a bond attached to A1; G^{B1} is a bond attached to Q; G^{B2} is a bond attached to A2 or M2 if A2 is absent; each of Z^{A1}, Z^{A2}, Z^{A3}, Z^{A4}, Z^{A5}, Z^{B1}, Z^{B2}, Z^{B3}, Z^{B4}, and Z^{B5} is independently, optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene; each of Y^{A1}, Y^{A2}, Y^{A3}, Y^{A4}, Y^{B1}, Y^{B2}, Y^{B3}, and Y^{B4} is, independently, O, S, NRⁱ, P, carbonyl, thiocarbonyl, sulfonyl, phosphate, phosphoryl, or imino; Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C3-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl; each of g1, h1, i1, j1, k1, l1, m1, n1, o1, g2, h2, i2, j2, k2, l2, m2, n2, and o2 is independently, 0 or 1; Q is a nitrogen atom, optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene.

Linkers of formula (D-L-II) that may be used in conjugates described herein include, but are not limited to,

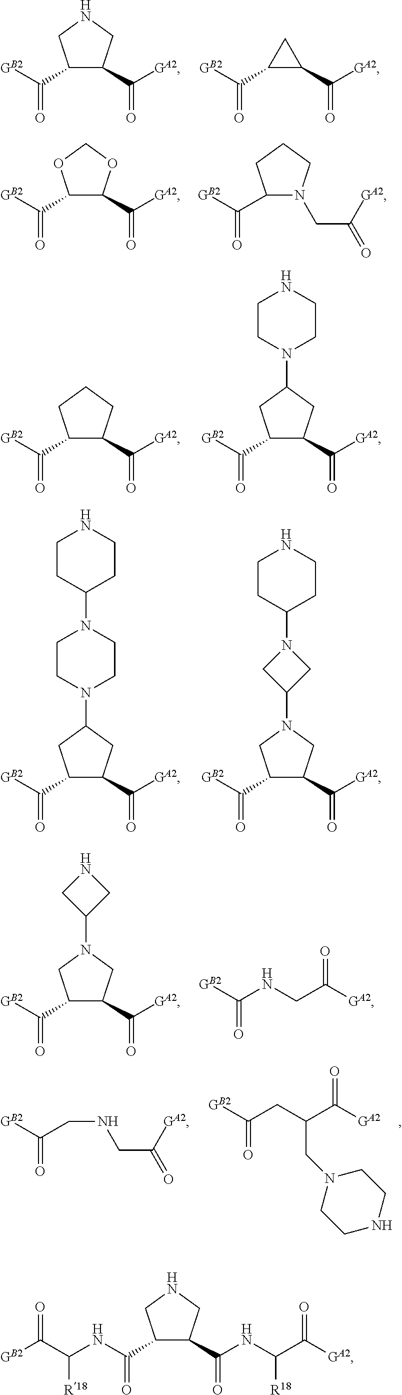

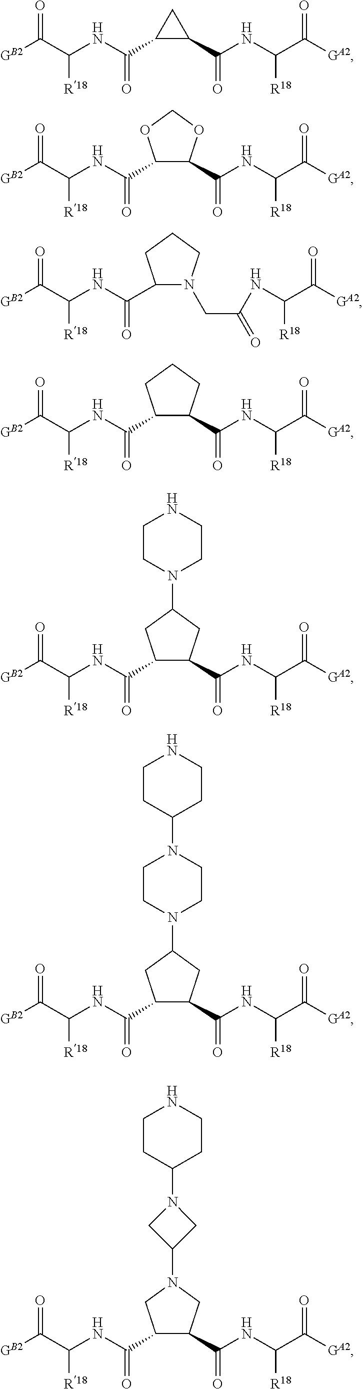

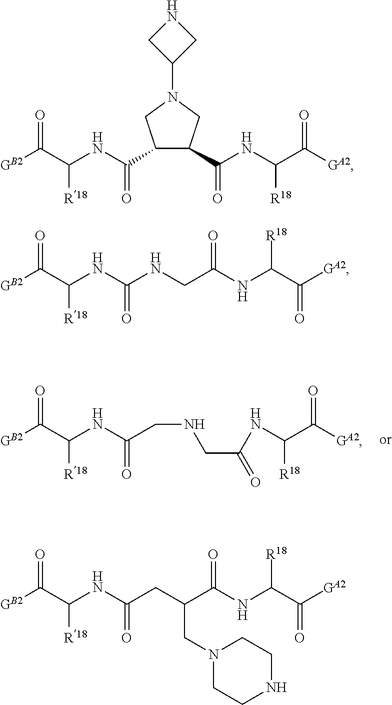

wherein each of R′¹⁸ and R¹⁸ is, independently, H, optionally substituted C1-C20 alkyl, optionally substituted C1-C20 heteroalkyl, optionally substituted C2-C20 akenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C3-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl. In some embodiments, each of R′¹⁸ and R^{1B} is, independently, butyl, cyclohexyl, isopropyl, or isobutyl.

In some embodiments, a linker in a conjugate having an Fc domain covalently linked to one or more dimers of cyclic heptapeptides is described by formula (D-L-III):

J¹-(S¹)_{g}-(T¹)ₕ-(S²)ᵢ-(T²)ⱼ-(S³)ₖ-(T³)ₗ-(S⁴)ₘ-(T⁴)ₙ-(S⁵)ₒ-J² (D-L-III)

wherein J¹ is a bond attached to A1; J² is a bond attached to E; each of S¹, S², S³, S⁴, and S⁵ is, independently, optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene; each of T¹, T², T³, T⁴ is, independently, O, S, NRⁱ, P, carbonyl, thiocarbonyl, sulfonyl, phosphate, phosphoryl, or imino; Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C3-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl; and each of g, h, i, j, k, l, m, n, and o is, independently, 0 or 1; or a pharmaceutically acceptable salt thereof.

Linkers of formula (D-L-III) that may be used in conjugates described herein include, but are not limited to,

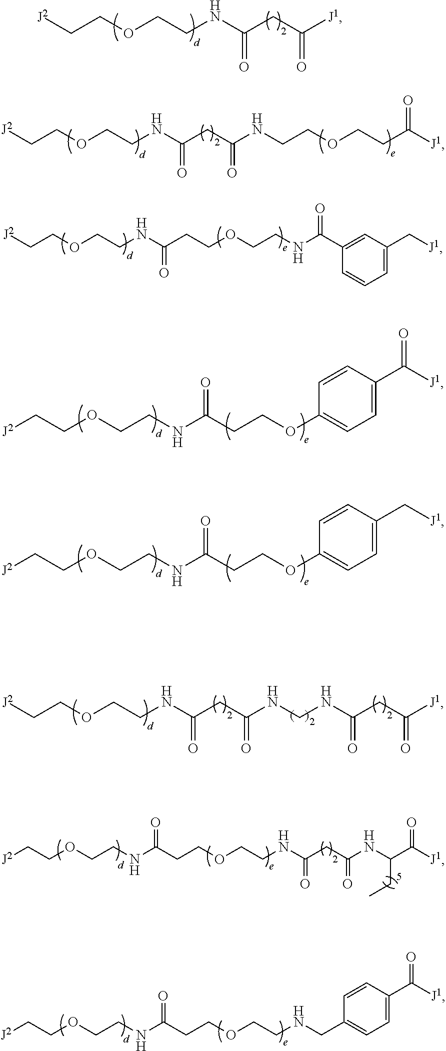

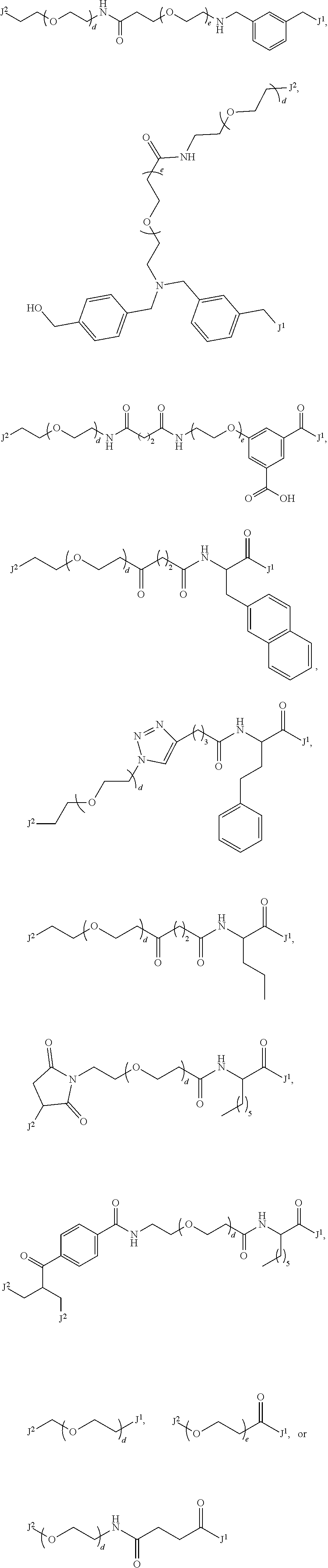

wherein each of d and e is, independently, an integer from 1 to 26; or a pharmaceutically acceptable salt thereof.

Linkers in Conjugates Having an Fc Domain Covalently Linked to Monomers of cyclic Heptapeptides

In a conjugate containing an Fc domain covalently linked to one or more monomers of cyclic heptapeptides as described herein, a linker in the conjugate (e.g., L′, or L) may be a divalent structure having two arms. One arm in a divalent linker may be attached to the monomer of cyclic heptapeptide and the arm may be attached to the Fc domain. In some embodiments, the one or more monomers of cyclic heptapeptides in the conjugates described herein may each be, independently, connected to an atom in the Fc domain.

In some embodiments, a linker is described by formula (M-L-I):

J¹-(Q¹)_{g}-(T¹)ₕ-(Q²)ᵢ-(T²)ⱼ-(Q³)ₖ-(T³)ₗ-(Q⁴)ₘ-(T⁴)ₙ-(Q⁵)ₒ-J² (M-L-I)

wherein J¹ is a bond attached to a cyclic heptapeptide or a peptide (e.g., a peptide including 1-5 amino acid residue(s)) attached to the cyclic heptapeptide, if the peptide is present; J² is a bond attached to an Fc domain; each of Q¹, Q², Q³, Q⁴, and Q⁵ is, independently, optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene; each of T¹, T², T³, T⁴ is, independently, O, S, NRⁱ, P, carbonyl, thiocarbonyl, sulfonyl, phosphate, phosphoryl, or imino; Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C3-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl; and each of g, h, i, j, k, l, m, n, and o is, independently, 0 or 1.

In some embodiments, depending on the structure and chemical formula of L-I, J² may have two points of attachment to the Fc domain (e.g., two J²).

Linkers of formula (L-I) that may be used in conjugates described herein include, but are not limited to,

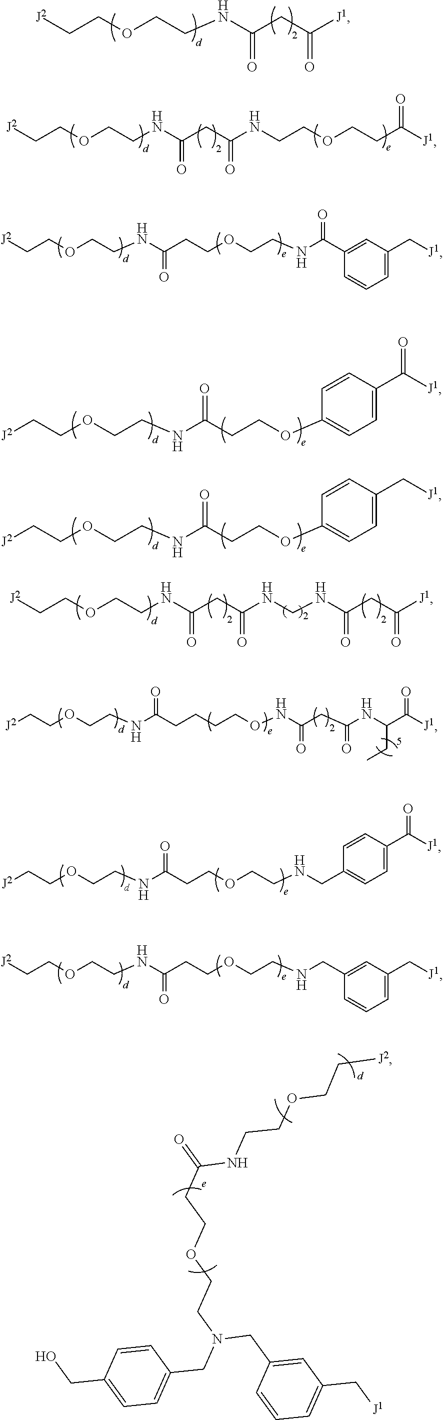

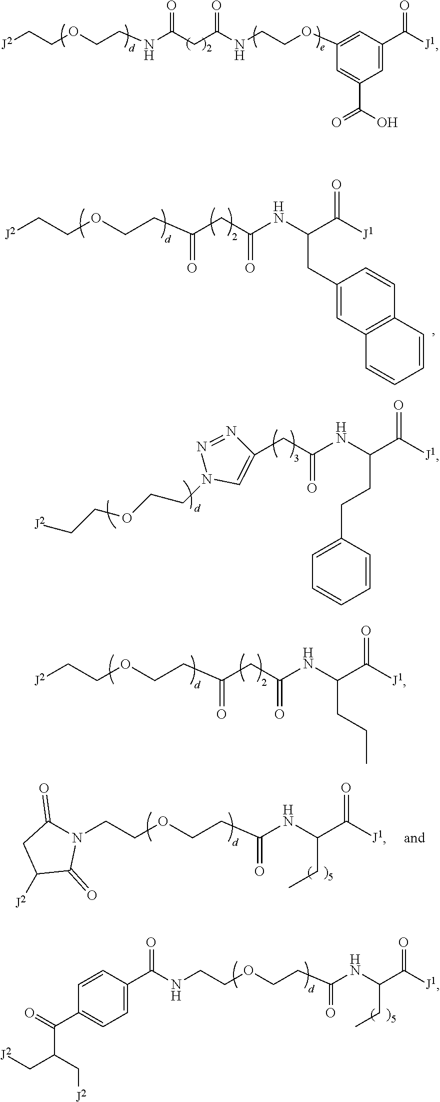

wherein each of d and e is, independently, an integer from 1 to 26.

In some embodiments, a linker provides space, rigidity, and/or flexibility between the cyclic heptapeptides and the Fc domain in the conjugates described here or between two cyclic heptapeptides in the conjugates described herein. In some embodiments, a linker may be a bond, e.g., a covalent bond, e.g., an amide bond, a disulfide bond, a C—O bond, a C—N bond, a N—N bond, a C—S bond, or any kind of bond created from a chemical reaction, e.g., chemical conjugation. In some embodiments, a linker (L′, L, or L¹ as shown in any one of formulas (I)-(XXXVI)) includes no more than 250 atoms (e.g., 1-2, 1-4, 1-6, 1-8, 1-10, 1-12, 1-14, 1-16, 1-18, 1-20, 1-25, 1-30, 1-35, 1-40, 1-45, 1-50, 1-55, 1-60, 1-65, 1-70, 1-75, 1-80, 1-85, 1-90, 1-95, 1-100, 1-110, 1-120, 1-130, 1-140, 1-150, 1-160, 1-170, 1-180, 1-190, 1-200, 1-210, 1-220, 1-230, 1-240, or 1-250 atom(s); 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 28, 26, 24, 22, 20, 18, 16, 14, 12, 10, 9, 8, 7, 6, 5, 4, 3, 2, or 1 atom(s)). In some embodiments, a linker (L′, L, or L¹) includes no more than 250 non-hydrogen atoms (e.g., 1-2, 1-4, 1-6, 1-8, 1-10, 1-12, 1-14, 1-16, 1-18, 1-20, 1-25, 1-30, 1-35, 1-40, 1-45, 1-50, 1-55, 1-60, 1-65, 1-70, 1-75, 1-80, 1-85, 1-90, 1-95, 1-100, 1-110, 1-120, 1-130, 1-140, 1-150, 1-160, 1-170, 1-180, 1-190, 1-200, 1-210, 1-220, 1-230, 1-240, or 1-250 non-hydrogen atom(s); 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 28, 26, 24, 22, 20, 18, 16, 14, 12, 10, 9, 8, 7, 6, 5, 4, 3, 2, or 1 non-hydrogen atom(s)). In some embodiments, the backbone of a linker (L′, L, or L¹) includes no more than 250 atoms (e.g., 1-2, 1-4, 1-6, 1-8, 1-10, 1-12, 1-14, 1-16, 1-18, 1-20, 1-25, 1-30, 1-35, 1-40, 1-45, 1-50, 1-55, 1-60, 1-65, 1-70, 1-75, 1-80, 1-85, 1-90, 1-95, 1-100, 1-110, 1-120, 1-130, 1-140, 1-150, 1-160, 1-170, 1-180, 1-190, 1-200, 1-210, 1-220, 1-230, 1-240, or 1-250 atom(s); 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 28, 26, 24, 22, 20, 18, 16, 14, 12, 10, 9, 8, 7, 6, 5, 4, 3, 2, or 1 atom(s)). The “backbone” of a linker refers to the atoms in the linker that together form the shortest path from one part of the conjugate to another part of the conjugate. The atoms in the backbone of the linker are directly involved in linking one part of the conjugate to another part of the conjugate. For examples, hydrogen atoms attached to carbons in the backbone of the linker are not considered as directly involved in linking one part of the conjugate to another part of the conjugate.

Molecules that may be used to make linkers (L′, L, or L¹) include at least two functional groups. e.g., two carboxylic acid groups. In some embodiments of a trivalent linker, two arms of a linker may contain two dicarboxylic acids, in which the first carboxylic acid may form a covalent linkage with the first cyclic heptapeptide in the conjugate and the second carboxylic acid may form a covalent linkage with the second cyclic heptapeptide in the conjugate, and the third arm of the linker may for a covalent linkage (e.g., a C—O bond) with an Fc domain in the conjugate. In some embodiments of a divalent linker, the divalent linker may contain two carboxylic acids, in which the first carboxylic acid may form a covalent linkage with one component (e.g., a cyclic heptapeptide) in the conjugate and the second carboxylic acid may form a covalent linkage (e.g., a C—S bond or a C—N bond) with another component (e.g., an Fc domain) in the conjugate. In some embodiments, when the cyclic heptapeptide is attached to a peptide (e.g., a peptide including a 1-5 amino acid residue(s)) at the linking nitrogen, the linker may form a covalent linkage with the peptide.

In some embodiments, dicarboxylic acid molecules may be used as linkers (e.g., a dicarboxylic acid linker). For example, in a conjugate containing an Fc domain covalently linked to one or more dimers of cyclic heptapeptides, the first carboxylic acid in a dicarboxylic acid molecule may form a covalent linkage with the linking nitrogen of the first cyclic heptapeptide and the second carboxylic acid may form a covalent linkage with the linking nitrogen of the second cyclic heptapeptide.

In some embodiments, when the first and/or second cyclic heptapeptide is attached to a peptide (e.g., a peptide including a 1-5 amino acid residue(s)) at the linking nitrogen, the first carboxylic acid in a dicarboxylic acid linker may form a covalent linkage with the terminal amine group at the end of the peptide that is attached to the first cyclic heptapeptide and the second carboxylic acid in the dicarboxylic acid linker may form a covalent linkage with the terminal amine group at the end of the peptide that is attached to the second cyclic heptapeptide.

Examples of dicarboxylic acids molecules that may be used to form linkers include, but are not limited to,

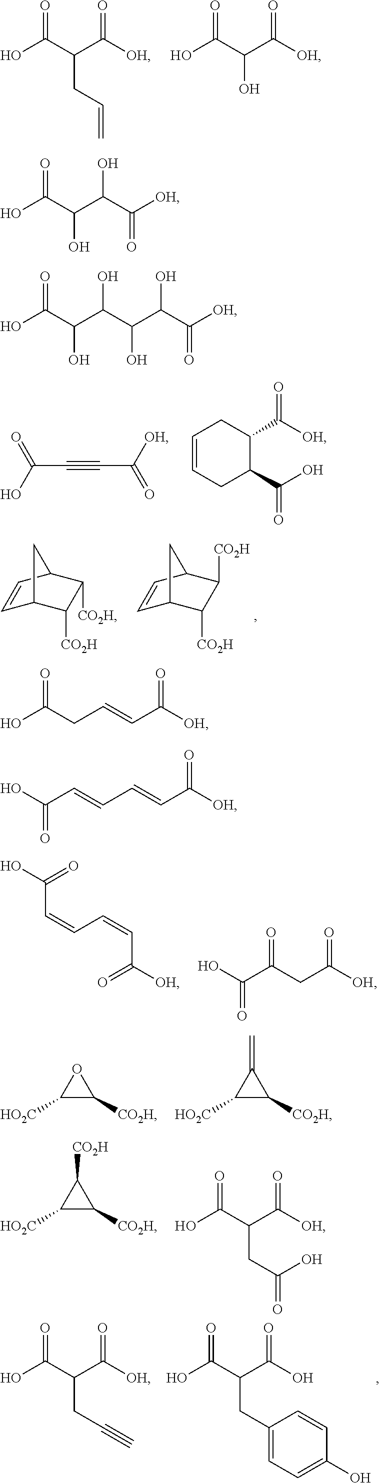

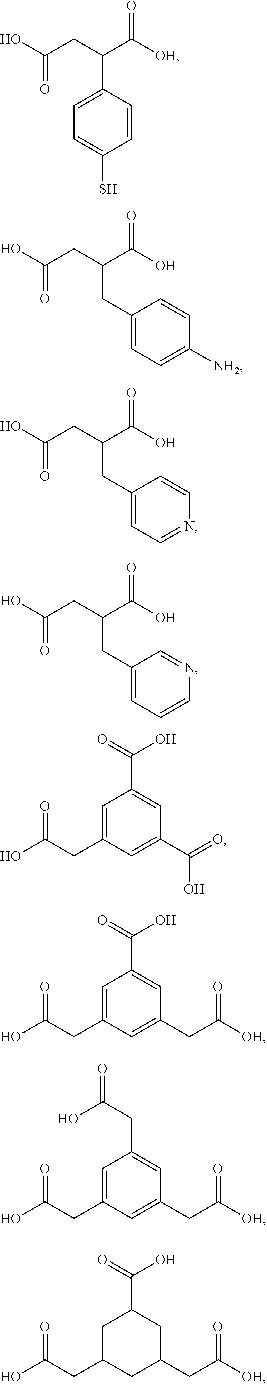

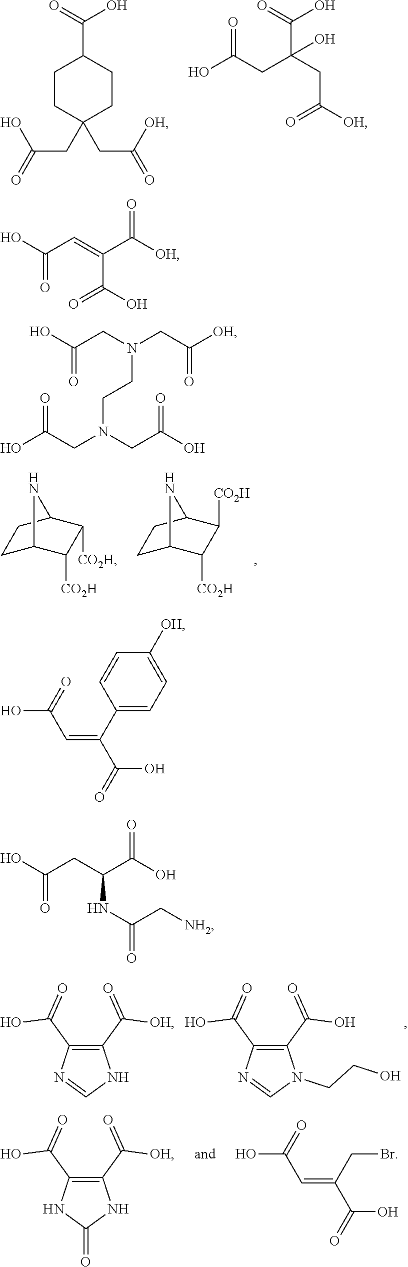

Other examples of dicarboxylic acids molecules that may be used to form linkers include, but are not limited to,

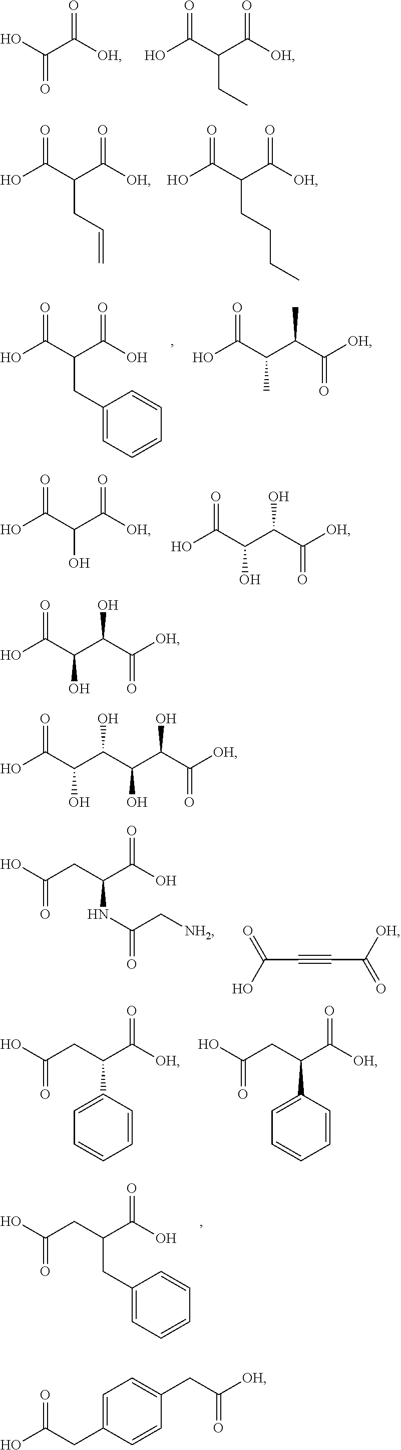

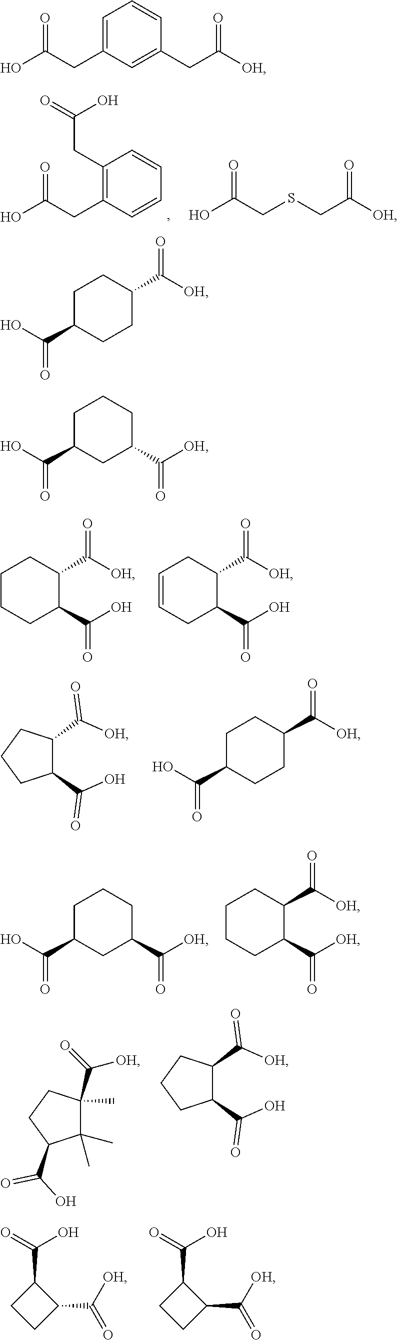

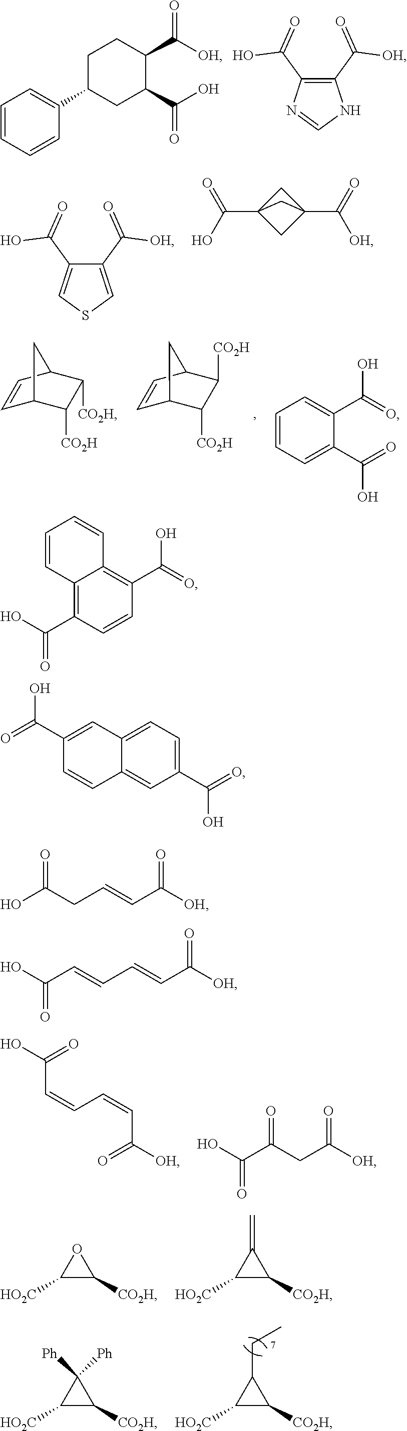

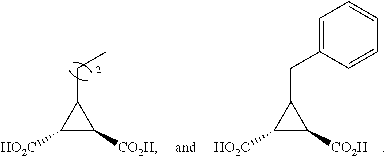

In some embodiments, dicarboxylic acid molecules, such as the ones described herein, may be further functionalized to contain one or more additional functional groups.

In some embodiments, when the cyclic heptapeptide is attached to a peptide (e.g., a peptide including a 1-5 amino acid residue(s)) at the linking nitrogen, the linking group may comprise a moiety comprising a carboxylic acid moiety and an amino moiety that are spaced by from 1 to 25 atoms. Examples of such linking groups include, but are not limited to,

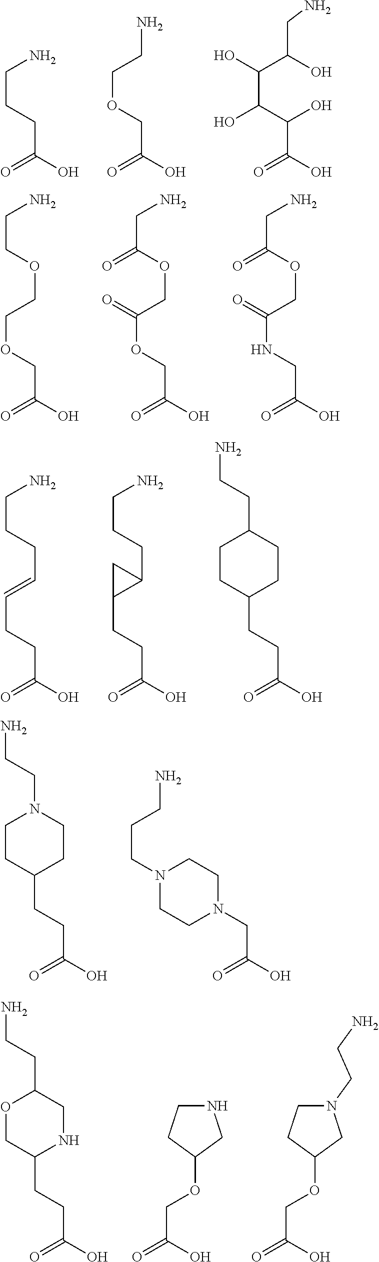

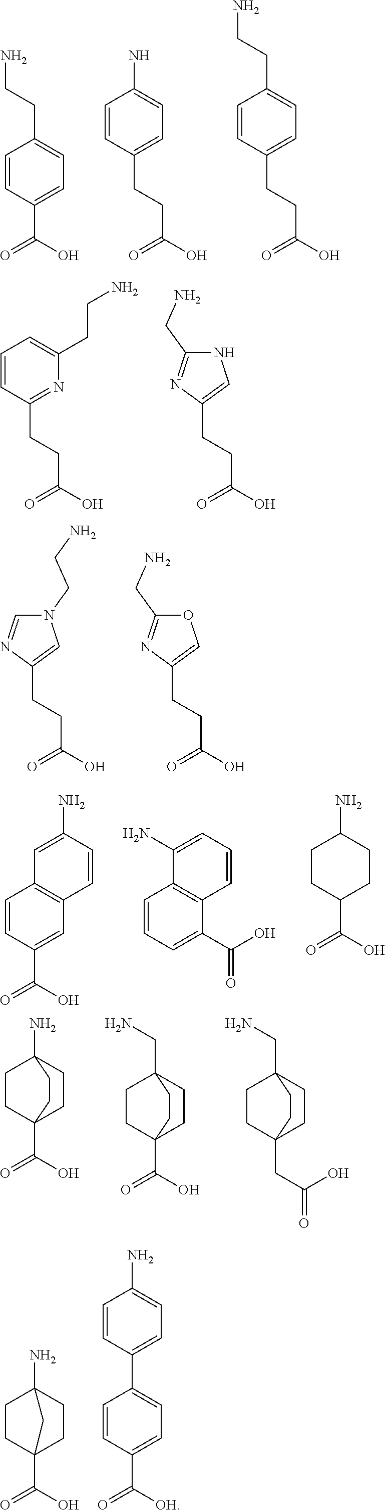

In some embodiments, a molecule containing an azide group may be used to form a linker, in which the azide group may undergo cycloaddition with an alkyne to form a 1,2,3-triazole linkage. In some embodiments, a molecule containing an alkyne group may be used to form a linker, in which the alkyne group may undergo cycloaddition with an azide to form a 1,2,3-triazole linkage. In some embodiments, a molecule containing a maleimide group may be used to form a linker, in which the maleimide group may react with a cysteine to form a C—S linkage. In some embodiments, a molecule containing one or more sulfonic acid groups may be used to form a linker, in which the sulfonic acid group may form a sulfonamide linkage with the linking nitrogen in a cyclic heptapeptide. In some embodiments, a molecule containing one or more isocyanate groups may be used to form a linker, in which the isocyanate group may form a urea linkage with the linking nitrogen in a cyclic heptapeptide. In some embodiments, a molecule containing one or more haloalkyl groups may be used to form a linker, in which the haloalkyl group may form a covalent linkage, e.g., C—N and C—O linkages, with a cyclic heptapeptide.

In some embodiments, a linker (L′, L, or L¹) may comprise a synthetic group derived from, e.g., a synthetic polymer (e.g., a polyethylene glycol (PEG) polymer). In some embodiments, a linker may comprise one or more amino acid residues. In some embodiments, a linker may be an amino acid sequence (e.g., a 1-25 amino acid, 1-10 amino acid, 1-9 amino acid, 1-8 amino acid, 1-7 amino acid, 1-6 amino acid, 1-5 amino acid, 1-4 amino acid, 1-3 amino acid, 1-2 amino acid, or 1 amino acid sequence). In some embodiments, a linker (L′, L, or L¹) may include one or more optionally substituted C1-C20 alkylene, optionally substituted C1-C20 heteroalkylene (e.g., a PEG unit), optionally substituted C2-C20 alkenylene (e.g., C2 alkenylene), optionally substituted C2-C20 heteroalkenylene, optionally substituted C2-C20 alkynylene, optionally substituted C2-C20 heteroalkynylene, optionally substituted C3-C20 cycloalkylene (e.g., cyclopropylene, cyclobutylene), optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene (e.g., C6 arylene), optionally substituted C2-C15 heteroarylene (e.g., imidazole, pyridine), O, S, NRⁱ (Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C3-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl), P, carbonyl, thiocarbonyl, sulfonyl, phosphate, phosphoryl, or imino.

### Conjugation Chemistries

Covalent conjugation of two or more components in a conjugate using a linker may be accomplished using well-known organic chemical synthesis techniques and methods. Complementary functional groups on two components may react with each other to form a covalent bond. Examples of complementary reactive functional groups include, but are not limited to, e.g., maleimide and cysteine, amine and activated carboxylic acid, thiol and maleimide, activated sulfonic acid and amine, isocyanate and amine, azide and alkyne, and alkene and tetrazine. Site-specific conjugation to a polypeptide (e.g., an Fc domain monomer or an Fc domain) may accomplished using techniques known in the art. Exemplary techniques for site-specific conjugation of a small molecule to an Fc domain are provided in Agarwall. P., et al. Bioconjugate Chem. 26:176-192 (2015).

Other examples of functional groups capable of reacting with amino groups include. e.g., alkylating and acylating agents. Representative alkylating agents include: (i) an α-haloacetyl group, e.g., XCH₂CO— (where X═Br, Cl, or I); (ii) a N-maleimide group, which may react with amino groups either through a Michael type reaction or through acylation by addition to the ring carbonyl group; (iii) an aryl halide, e.g., a nitrohaloaromatic group; (iv) an alkyl halide; (v) an aldehyde or ketone capable of Schiff's base formation with amino groups; (vi) an epoxide, e.g., an epichlorohydrin and a bisoxirane, which may react with amino, sulfhydryl, or phenolic hydroxyl groups; (vii) a chlorine-containing of s-triazine, which is reactive towards nucleophiles such as amino, sufhydryl, and hydroxyl groups; (viii) an aziridine, which is reactive towards nucleophiles such as amino groups by ring opening; (ix) a squaric acid diethyl ester; and (x) an α-haloalkyl ether.

Examples of amino-reactive acylating groups include, e.g., (i) an isocyanate and an isothiocyanate; (ii) a sulfonyl chloride; (iii) an acid halide; (iv) an active ester, e.g., a nitrophenylester or N-hydroxysuccinimidyl ester; (v) an acid anhydride, e.g., a mixed, symmetrical, or N-carboxyanhydride; (vi) an acylazide; and (vii) an imidoester. Aldehydes and ketones may be reacted with amines to form Schiff's bases, which may be stabilized through reductive amination.

It will be appreciated that certain functional groups may be converted to other functional groups prior to reaction, for example, to confer additional reactivity or selectivity. Examples of methods useful for this purpose include conversion of amines to carboxyls using reagents such as dicarboxylic anhydrides; conversion of amines to thiols using reagents such as N-acetylhomocysteine thiolactone, S-acetylmercaptosuccinic anhydride, 2-iminothiolane, or thiol-containing succinimidyl derivatives; conversion of thiols to carboxyls using reagents such as α-haloacetates; conversion of thiols to amines using reagents such as ethylenimine or 2-bromoethylamine; conversion of carboxyls to amines using reagents such as carbodiimides followed by diamines; and conversion of alcohols to thiols using reagents such as tosyl chloride followed by transesterification with thioacetate and hydrolysis to the thiol with sodium acetate.

In some embodiments, a linker of the invention (e.g., L or L′, such as L^{C} of D-L-I), is conjugated (e.g., by any of the methods described herein) to E (e.g., an Fc domain monomer or an Fc domain). In preferred embodiments of the invention, the linker is conjugated via: (a) a thiourea linkage (i.e., —NH(C═S)NH—) to a lysine of E; (b) a carbamate linkage (i.e., —NH(C═O)—O—) to a lysine of E; (c) an amine linkage by reductive amination (i.e., —NHCH₂—) between a lysine and E; (d) an amide (i.e., —NH(C═O)CH₂—) to a lysine of E; (e) a cysteine-maleimide conjugate between a maleimide of the linker to a cysteine of E; (f) an amine linkage by reductive amination (i.e., —NHCH₂—) between the linker and a carbohydrate of E (e.g., a glycosyl group of an Fc domain monomer or an Fc domain); (g) a rebridged cysteine conjugate, wherein the linker is conjugated to two cysteines of E; (h) an oxime linkage between the linker and a carbohydrate of E (e.g., a glycosyl group of an Fc domain monomer or an Fc domain); (i) an oxime linkage between the linker and an amino acid residue of E; (j) an azido linkage between the linker and E; (k) direct acylation of a linker to E; or (I) a thioether linkage between the linker and E.

In some embodiments, a linker is conjugated to E. wherein the linkage includes the structure —NH(C═NH)X—, wherein X is O, HN, or a bond. In some embodiments, a linker is conjugated to E. wherein the linkage between the remainder of the linker and E includes the structure —NH(C═O)NH—.

Exemplary linking strategies (e.g., methods for linking a polymyxin monomer or a dimer to E, such as, via a linker) are further depicted in FIGS. 114-117.

### V. Antibacterial Agents

In some embodiments, one or more antibacterial agents may be administered in combination (e.g., administered substantially simultaneously (e.g., in the same pharmaceutical composition or in separate pharmaceutical compositions) or administered separately at different times) with a conjugate described herein (e.g., a conjugate of any one of formulas (I)-(XXXVI)).

Antibacterial agents may be grouped into several classes, e.g., quinolones, carbapenems, macrolides, DHFR inhibitors, aminoglycosides, ansamycins (e.g., geldanamycin, herimycin, and rifaximin), carbacephem (e.g., loracarbef), cephalosporins (e.g., cefadroxil, cefaolin, cefalotin, cefalothin, cephalexin, e.g., cefaclor, cefamandole, cefoxitin, cefprozil, cefuroxime, cefdinir, cefditoren, cefoperazone, cefotaxime, cefpodoxime, ceftazidime, and ceftobiprole), glycopeptides (e.g., teicoplanin, vancomycin, telavancin, dalbavancin, and oritavancin), lincosamides (e.g., clindamycin and lincomycin), lipopeptides (e.g., daptomycin), monobactams (e.g., aztreonam), nitrofurans (e.g., furazolidone and nitrofurantoin), oxazolidinones, pleuromutilins, penicillins, sulfonamides, and tetracyclines (e.g., eravacycline, demeclocycline, doxycycline, minocycline, oxytetracycline, and tetracycline). Quinolones include, but are not limited to, ciprofloxacin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin, and temafloxacin. Carbapenems include, but are not limited to, ertapenem, doripenem, imipenem/cilastatin, and meropenem. Macrolides include, but are not limited to, solithromycin, azithromycin, clarithromycin, dirithromycin, erythromycin, roxithromycin, troleandomycin, telithromycin, and spiramycin. In some embodiments, a macrolide is solithromycin. DHFR inhibitors include, but are not limited to, diaminoquinazoline, diaminopyrroloquinazoline, diaminopyrimidine, diaminopteridine, and diaminotriazines. Aminoglycosides include, but are not limited to, plazomicin, amikacin, gentamicin, gamithromycin, kanamycin, neomycin, netilmicin, tobramycin, paromomycin, streptomycin, and spectinomycin. Oxazolidinones include, but are not limited to, linezolid, tedizolid, posizolid, radezolid, and furazolidone. Pleuromutilins include, but are not limited to, retapamulin, valnemulin, tiamulin, azamulin, and lefamulin. Penicillins include, but are not limited to, amoxicillin, ampicillin, azlocillin, carbenicillin, cloxacillin, dicloxacillin, flucloxacillin, mezlocillin, methicillin, nafcillin, oxacillin, penicillin G, penicillin V, piperacillin, penicillin G, temocillin, and ticarcillin. Sulfonamides include, but are not limited to, mafenide, sulfacetamide, sulfadiazine, silver sulfadiazine, sulfadimethoxine, sulfamethizole, sulfamethoxazole, sulfanilimide, sulfasalazine, sulfisoxazole, trimethoprim-sulfamethoxazole (Co-trimoxazole) (TMP-SMX), and sulfonamidochrysoidine.

In some embodiments, the antibacterial agent used in combination with a conjugate described herein (e.g., a conjugate of any one of formulas (I)-(XXXVI)) is selected from the group consisting of linezolid, tedizolid, posizolid, radezolid, retapamulin, valnemulin, tiamulin, azamulin, lefamulin, plazomicin, amikacin, gentamicin, gamithromycin, kanamycin, neomycin, netilmicin, tobramycin, paromomycin, streptomycin, spectinomycin, geldanamycin, herbimycin, rifaximin, loracarbef, ertapenem, doripenem, imipenem/cilastatin, meropenem, cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil, ceftobiprole, teicoplanin, vancomycin, telavancin, dalbavancin, oritavancin, clindamycin, lincomycin, daptomycin, solithromycin, azithromycin, clarithromycin, dirithromycin, erythromycin, roxithromycin, troleandomycin, telithromycin, spiramycin, aztreonam, furazolidone, nitrofurantoin, amoxicillin, ampicillin, azlocillin, carbenicillin, cloxacillin, dicloxacillin, flucloxacillin, mezlocillin, methicillin, nafcillin, oxacillin, penicillin g, penicillin v, piperacillin, penicillin g, temocillin, ticarcillin, amoxicillin clavulanate, ampicillin/sulbactam, piperacillin/tazobactam, ticarcillin/clavulanate, bacitracin, ciprofloxacin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfacetamide, sulfadiazine, silver sulfadiazine, sulfadimethoxine, sulfamethizole, sulfamethoxazole, sulfanilimide, sulfasalazine, sulfisoxazole, trimethoprim-sulfamethoxazole (tmp-smx), sulfonamidochrysoidine, eravacycline, demeclocycline, doxycycline, minocycline, oxytetracycline, tetracycline, clofazimine, dapsone, capreomycin, cycloserine, ethambutol(bs), ethionamide, isoniazid, pyrazinamide, rifampicin, rifabutin, rifapentine, streptomycin, arsphenamine, chloramphenicol, fosfomycin, fusidic acid, metronidazole, mupirocin, platensimycin, quinupristin/dalfopristin, thiamphenicol, tigecycline, tinidazole, and trimethoprim, prodrugs thereof, and pharmaceutically acceptable salts thereof.

In some embodiments, the antibacterial agent used in combination with a conjugate described herein (e.g., a conjugate of any one of formulas (I)-(XXXVI)) is tedizolid, azithromycin, meropenem, amikacin, levofloxacin, rifampicin, linezolid, erythromycin, or solithromycin. In some embodiments, the antibacterial agent used in combination with a conjugate described herein (e.g., a conjugate of any one of formulas (I)-(XXXVI)) is tedizolid, azithromycin, meropenem, amikacin, or levofloxacin. In some embodiments, the antibacterial agent used in combination with a conjugate described herein (e.g., a conjugate of any one of formulas (I)-(XXXVI)) is tiamulin. In some embodiments, the antibacterial agent used in combination with a conjugate described herein (e.g., a conjugate of any one of formulas (I)-(XXXVI)) is solithromycin.

### VI. Methods

Methods described herein include, e.g., methods of protecting against or treating a bacterial infection (e.g., a Gram-negative bacterial infection) in a subject and methods of preventing, stabilizing, or inhibiting the growth of bacteria, or killing bacteria (e.g., Gram-negative bacteria). A method of treating a bacterial infection (e.g., a Gram-negative bacterial infection) in a subject includes administering to the subject a conjugate described herein (e.g., a conjugate of any one of formulas (I)-(XXXVI)) or a pharmaceutical composition thereof. In some embodiments, the bacterial infection is caused by Gram-negative bacteria. In some embodiments, the bacterial infection is caused by a resistant strain of bacteria. In some embodiments, the resistant strain of bacteria is a resistant strain of E. coli. A method of preventing, stabilizing, or inhibiting the growth of bacteria, or killing bacteria (e.g., Gram-negative bacteria) includes contacting the bacteria (e.g., Gram-negative bacteria) or a site susceptible to bacterial growth (e.g., Gram-negative bacterial growth) with a conjugate described herein (e.g., a conjugate of any one of formulas (I)-(XXXVI)) or a pharmaceutical composition thereof. In some embodiments, the bacterial infection is caused by Gram-negative bacteria. In some embodiments, the bacterial infection is caused by a resistant strain of bacteria. In some embodiments, the resistant strain of bacteria is a resistant strain of E. coli. In some embodiments, a conjugate used in any methods described herein (e.g., a conjugate of any one of formulas (I)-(XXXVI)) may bind to LPS in the cell membrane of Gram-negative bacteria to disrupt and permeabilize the cell membrane, leading to cell death and/or sensitization to other antibiotics. Furthermore, in addition to disrupting the cell membrane of Gram-negative bacteria, the Fc domain in the conjugates described herein binds to the FcγRs (e.g., FcRn, FcγRI, FcγRIIa, FcγRIIc, FcγRIIIa, and FcγRIIIb) on immune cells and serves as a gradient against which immune cells chemotax to the site of bacterial infection and/or growth. The binding of the Fc domain in the conjugates described herein to the FcγRs on immune cells activates phagocytosis and effector functions, such as antibody-dependent cell-mediated cytotoxicity (ADCC), thus leading to the engulfment and destruction of Gram-negative bacterial cells by immune cells and further enhancing the antibacterial activity of the conjugates.

Moreover, methods described herein also include methods of protecting against or treating sepsis in a subject by administering to the subject a conjugate described herein (e.g., a conjugate of any one of formulas (I)-(XXXVI)). In some embodiments, the method further includes administering to the subject an antibacterial agent. Methods described herein also include methods of preventing lipopolysaccharides (LPS) in Gram-negative bacteria (e.g., a resistant strain of Gram-negative bacteria or a resistant strain of E. coli(e.g., E. coli BAA-2469)) from activating a immune system in a subject by administering to the subject a conjugate described herein (e.g., a conjugate of any one of formulas (I)-(XXXVI)). In some embodiments of the method, the method prevents LPS from activating a macrophage. In some embodiments, the method further includes administering to the subject an antibacterial agent. In some embodiments, a conjugate used in any methods described herein (e.g., a conjugate of any one of formulas (I)-(XXXVI)) may bind to LPS in the cell membrane of Gram-negative bacteria to disrupt and permeabilize the cell membrane, leading to cell death and/or sensitization to other antibiotics.

In some embodiments, the methods described herein may further include administering to the subject an antibacterial agent in addition to a conjugate described herein (e.g., a conjugate of any one of formulas (I)-(XXXVI)). Methods described herein also include methods of protecting against or treating a bacterial infection in a subject by administering to said subject (1) a conjugate described herein (e.g., a conjugate of any one of formulas (I)-(XXXVI)) and (2) an antibacterial agent. Methods described herein also include methods of preventing, stabilizing, or inhibiting the growth of bacteria, or killing bacteria, by contacting the bacteria or a site susceptible to bacterial growth with (1) a conjugate described herein (e.g., a conjugate of any one of formulas (I)-(XXXVI)) and (2) an antibacterial agent.

In some embodiments, the conjugate described herein (e.g., a conjugate of any one of formulas (I)-(XXXVI)) is administered first, followed by administering of the antibacterial agent alone. In some embodiments, the antibacterial agent is administered first, followed by administering of the conjugate described herein alone. In some embodiments, the conjugate described herein and the antibacterial agent are administered substantially simultaneously (e.g., in the same pharmaceutical composition or in separate pharmaceutical compositions). In some embodiments, the conjugate described herein or the antibacterial agent is administered first, followed by administering of the conjugate described herein and the antibacterial agent substantially simultaneously (e.g., in the same pharmaceutical composition or in separate pharmaceutical compositions). In some embodiments, the conjugate described herein and the antibacterial agent are administered first substantially simultaneously (e.g., in the same pharmaceutical composition or in separate pharmaceutical compositions), followed by administering of the conjugate described herein or the antibacterial agent alone. In some embodiments, when a conjugate described herein (e.g., a conjugate of any one of formulas (I)-(XXXVI)) and an antibacterial agent are administered together (e.g., substantially simultaneously in the same or separate pharmaceutical compositions, or separately in the same treatment regimen), the MIC of each of the conjugate and the antibacterial agent may be lower than the MIC of each of the conjugate and the antibacterial agent when each is used alone in a treatment regimen.

### VII. Pharmaceutical Compositions and Preparations

A conjugate described herein may be formulated in a pharmaceutical composition for use in the methods described herein. In some embodiments, a conjugate described herein may be formulated in a pharmaceutical composition alone. In some embodiments, a conjugate described herein may be formulated in combination with an antibacterial agent in a pharmaceutical composition. In some embodiments, the pharmaceutical composition includes a conjugate described herein (e.g., a conjugate described by any one of formulas (I)-(XXXVI)) and pharmaceutically acceptable carriers and excipients. Acceptable carriers and excipients in the pharmaceutical compositions are nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acid residues such as glycine, glutamine, histidine, and lysine, and carbohydrates such as glucose, mannose, sucrose, and sorbitol.

Examples of other excipients include, but are not limited to, antiadherents, binders, coatings, compression aids, disintegrants, dyes, emollients, emulsifiers, fillers (diluents), film formers or coatings, flavors, fragrances, glidants (flow enhancers), lubricants, sorbents, suspensing or dispersing agents, or sweeteners. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The conjugates herein may have ionizable groups so as to be capable of preparation as pharmaceutically acceptable salts. These salts may be acid addition salts involving inorganic or organic acids or the salts may, in the case of acidic forms of the conjugates herein be prepared from inorganic or organic bases. Frequently, the conjugates are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art.

Representative acid addition salts include, but are not limited to, acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include, but are not limited to, sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

Depending on the route of administration and the dosage, a conjugate herein or a pharmaceutical composition thereof used in the methods described herein will be formulated into suitable pharmaceutical compositions to permit facile delivery. A conjugate (e.g., a conjugate of any one of formulas (I)-(XXXVI)) or a pharmaceutical composition thereof may be formulated to be administered intramuscularly, intravenously (e.g., as a sterile solution and in a solvent system suitable for intravenous use), intradermally, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctival, intravesicularlly, mucosally, intrapericardially, intraumbilically, intraocularally, orally (e.g., a tablet, capsule, caplet, gelcap, or syrup), topically (e.g., as a cream, gel, lotion, or ointment), locally, by inhalation, by injection, or by infusion (e.g., continuous infusion, localized perfusion bathing target cells directly, catheter, lavage, in cremes, or lipid compositions). Depending on the route of administration, a conjugate herein or a pharmaceutical composition thereof may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

A conjugate described herein may be formulated in a variety of ways that are known in the art. For use as treatment of human and animal subjects, a conjugate described herein can be formulated as pharmaceutical or veterinary compositions. Depending on the subject (e.g., a human) to be treated, the mode of administration, and the type of treatment desired, e.g., prophylaxis or therapy, a conjugate described herein is formulated in ways consonant with these parameters. A summary of such techniques is found in Remington: The Science and Practice of Pharmacy, 22nd Edition, Lippincott Williams & Wilkins (2012); and Encyclopedia of Pharmaceutical Technology, 4th Edition, J. Swarbrick and J. C. Boylan, Marcel Dekker, New York (2013), each of which is incorporated herein by reference. Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. The formulation will generally include a diluent as well as, in some cases, adjuvants, buffers, and preservatives. The conjugates can be administered also in liposomal compositions or as microemulsions. Systemic administration may also include relatively noninvasive methods such as the use of suppositories, transdermal patches, transmucosal delivery and intranasal administration. Oral administration is also suitable for conjugates herein. Suitable forms include syrups, capsules, and tablets, as is understood in the art.

The pharmaceutical compositions can be administered parenterally in the form of an injectable formulation. Pharmaceutical compositions for injection can be formulated using a sterile solution or any pharmaceutically acceptable liquid as a vehicle. Formulations may be prepared as solid forms suitable for solution or suspension in liquid prior to injection or as emulsions. Pharmaceutically acceptable vehicles include, but are not limited to, sterile water, physiological saline, and cell culture media (e.g., Dulbecco's Modified Eagle Medium (DMEM), α-Modified Eagles Medium (α-MEM), F-12 medium). Such injectable compositions may also contain amounts of nontoxic auxiliary substances such as wetting or emulsifying agents, pH buffering agents, such as sodium acetate and sorbitan monolaurate. Formulation methods are known in the art, see e.g., Pharmaceutical Preformulation and Formulation, 2nd Edition, M. Gibson, Taylor & Francis Group, CRC Press (2009).

The pharmaceutical compositions can be prepared in the form of an oral formulation. Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Other pharmaceutically acceptable excipients for oral formulations include, but are not limited to, colorants, flavoring agents, plasticizers, humectants, and buffering agents. Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion controlled release of a conjugate described herein (e.g., a conjugate of any one of formulas (I)-(XXXVI)) or a pharmaceutical composition thereof can be achieved by appropriate coating of a tablet, capsule, pellet, or granulate formulation of the conjugate, or by incorporating the conjugate into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, caranuba wax and stearyl alcohol, carbopol 934, silicone, glyceryl tristearate, methyl acrylate-methyl methacrylate, polyvinyl chloride, polyethylene, and/or halogenated fluorocarbon.

The pharmaceutical composition may be formed in a unit dose form as needed. The amount of active component, e.g., a conjugate described herein (e.g., a conjugate of any one of formulas (I)-(XXXVI)), included in the pharmaceutical compositions are such that a suitable dose within the designated range is provided (e.g., a dose within the range of 0.01-100 mg/kg of body weight).

### VII. Routes of Administration and Dosages

In any of the methods described herein, conjugates herein may be administered by any appropriate route for treating or protecting against a bacterial infection (e.g., a Gram-negative bacterial infection), or for preventing, stabilizing, or inhibiting the growth of bacteria, or killing bacteria (e.g., Gram-negative bacteria). Conjugates described herein may be administered to humans, domestic pets, livestock, or other animals with a pharmaceutically acceptable diluent, carrier, or excipient. In some embodiments, administering comprises administration of any of the conjugates described herein (e.g., conjugates of any one of formulas (I)-(XXXVI)) or compositions intramuscularly, intravenously (e.g., as a sterile solution and in a solvent system suitable for intravenous use), intradermally, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctival, intravesicularlly, mucosally, intrapericardially, intraumbilically, intraocularally, orally (e.g., a tablet, capsule, caplet, gelcap, or syrup), topically (e.g., as a cream, gel, lotion, or ointment), locally, by inhalation, by injection, or by infusion (e.g., continuous infusion, localized perfusion bathing target cells directly, catheter, lavage, in cremes, or lipid compositions). In some embodiments, if an antibacterial agent is also administered in addition to a conjugate described herein, the antibacterial agent or a pharmaceutical composition thereof may also be administered in any of the routes of administration described herein.

The dosage of a conjugate described herein (e.g., a conjugate of any one of formulas (I)-(XXXVI)) or pharmaceutical compositions thereof depends on factors including the route of administration, the disease to be treated (e.g., the extent and/or condition of the bacterial infection), and physical characteristics, e.g., age, weight, general health, of the subject. Typically, the amount of the conjugate or the pharmaceutical composition thereof contained within a single dose may be an amount that effectively prevents, delays, or treats the bacterial infection without inducing significant toxicity. A pharmaceutical composition may include a dosage of a conjugate described herein ranging from 0.01 to 500 mg/kg (e.g., 0.01, 0.1, 0.2, 0.3, 0.4, 0.5, 1, 2, 3, 4, 5, 10, 15, 20, 25, 30, 35, 40, 45, 50, 100, 150, 200, 250, 300, 350, 400, 450, or 500 mg/kg) and, in a more specific embodiment, about 0.1 to about 30 mg/kg and, in a more specific embodiment, about 1 to about 30 mg/kg. In some embodiments, when a conjugate described herein (e.g., a conjugate of any one of formulas (I)-(XXXVI)) and an antibacterial agent are administered together (e.g., substantially simultaneously in the same or separate pharmaceutical compositions, or separately in the same treatment regimen), the dosage needed of the conjugate described herein may be lower than the dosage needed of the conjugate if the conjugate was used alone in a treatment regimen.

A conjugate described herein (e.g., a conjugate of any one of formulas (I)-(XXXVI)) or a pharmaceutical composition thereof may be administered to a subject in need thereof, for example, one or more times (e.g., 1-10 times or more; 1, 2, 3, 4, 5, 6, 7, 8, 9, or 10 times) daily, weekly, monthly, biannually, annually, or as medically necessary. Dosages may be provided in either a single or multiple dosage regimens. The timing between administrations may decrease as the medical condition improves or increase as the health of the patient declines. The dosage and frequency of administration may be adapted by the physician in accordance with conventional factors such as the extent of the infection and different parameters of the subject.

### EXAMPLES

### Example 1—Preparation of Fc Constructs

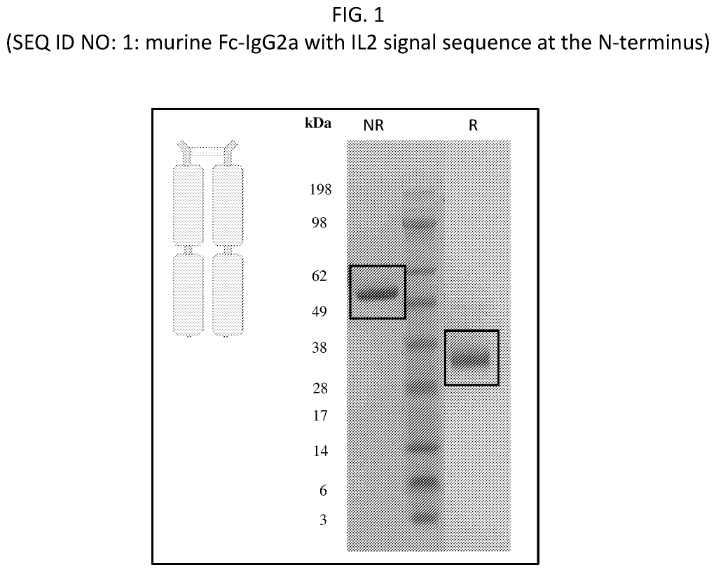

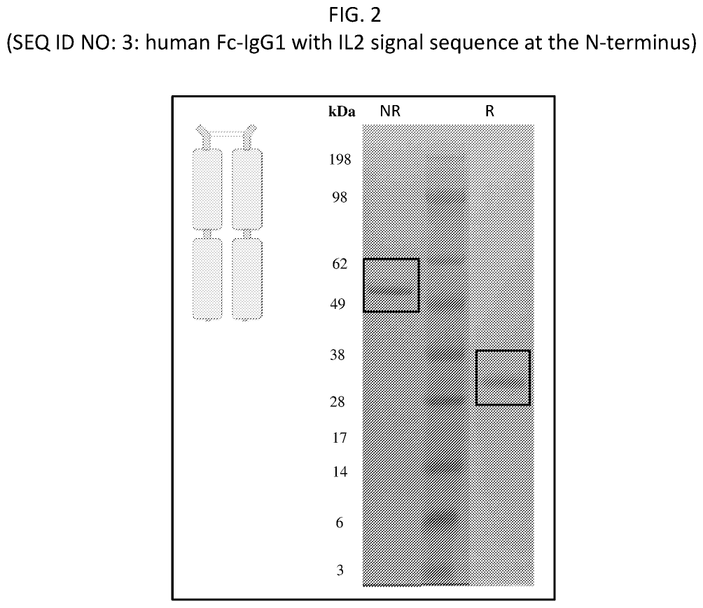

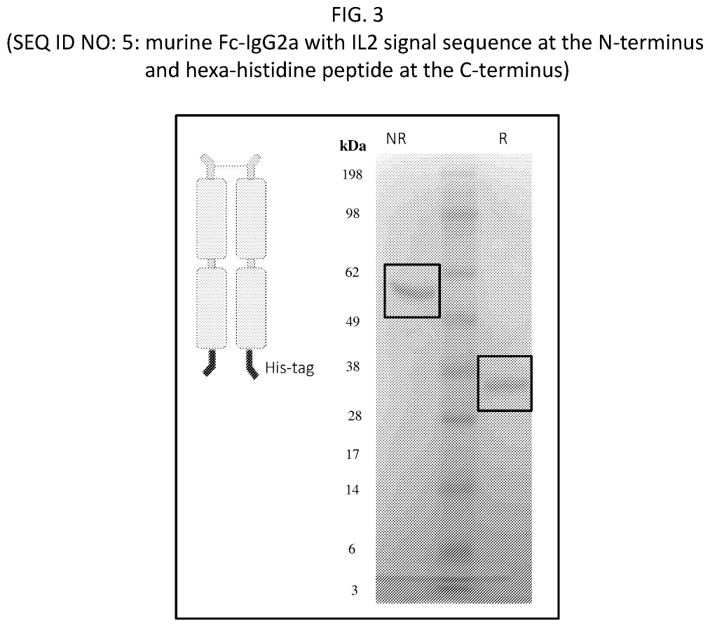

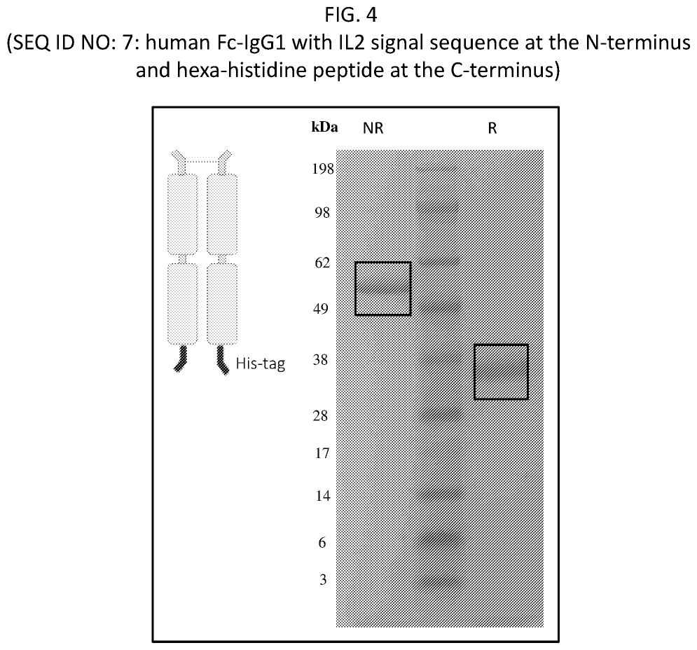

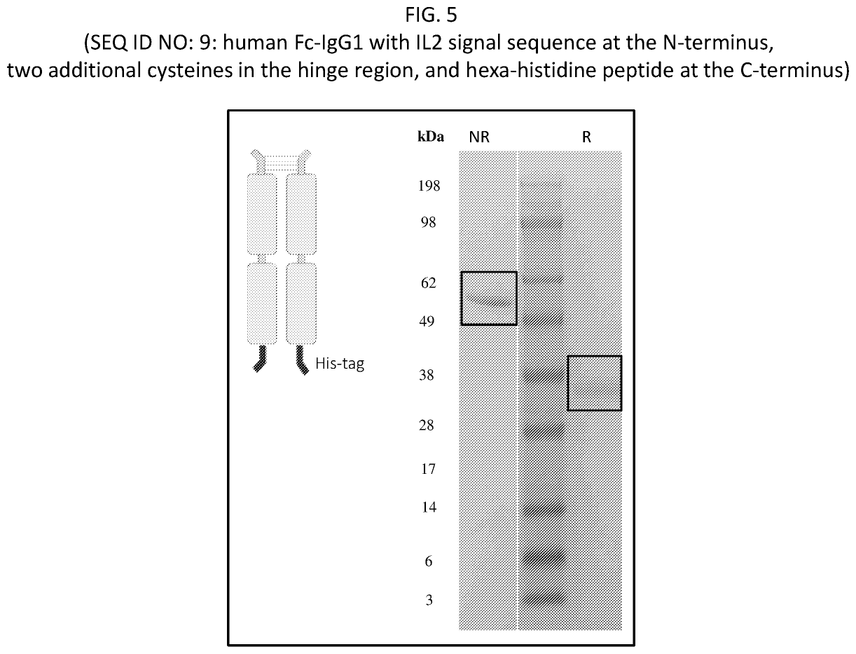

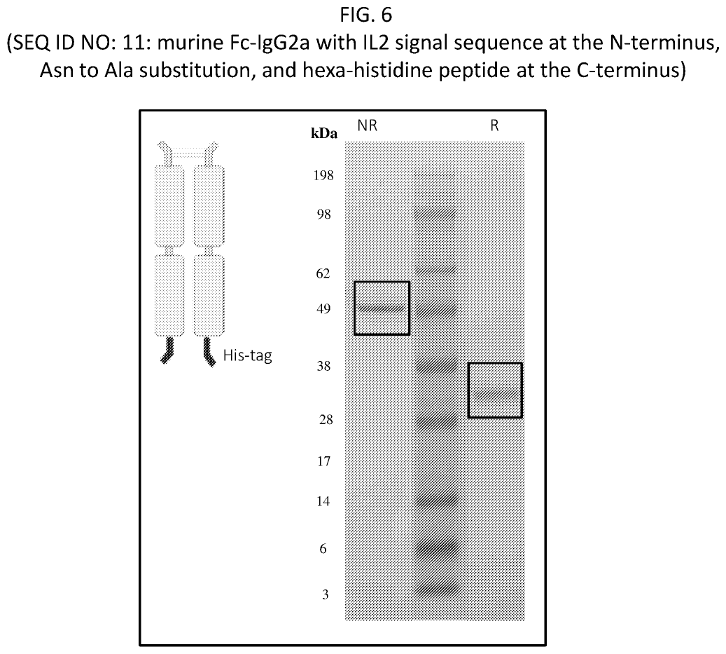

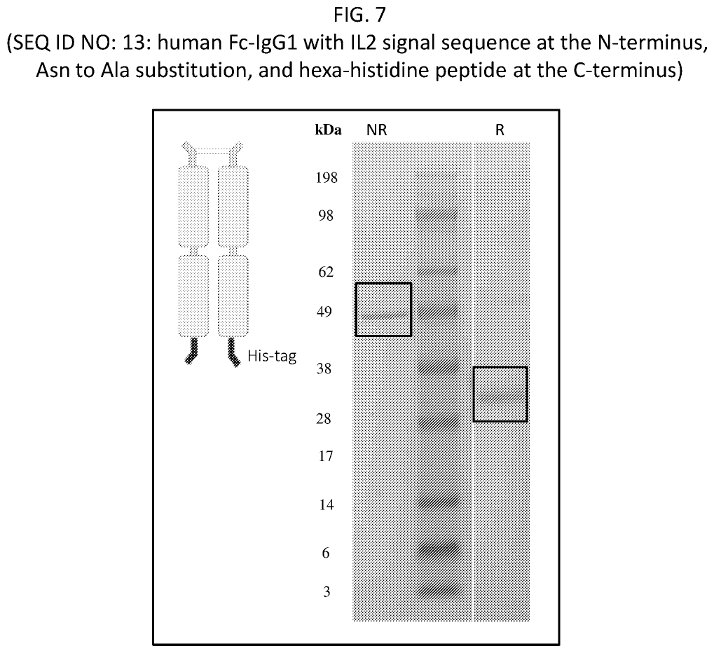

Reverse translations of the amino acids comprising the protein constructs (SEQ ID NOs: 1, 3, 5, 7, 9, 11, and 13) were synthesized by solid-phase synthesis. The oligonucleotide templates were cloned into pcDNA3.1 (Life Technologies. Carlsbad, Calif., USA) at the cloning sites BamHI and XhoI (New England Biolabs, Ipswich, Mass., USA) and included signal sequences derived from the human Interleukin-2 or human albumin. The pcDNA3.1 plasmids were transformed into Top 10 E. coli cells (LifeTech). DNA was amplified, extracted, and purified using the PureLink® HiPure Plasmid Filter Maxiprep Kit (LifeTech). The plasmid DNA is delivered, using the ExpiFectamine™ 293 Transfection Kit (LifeTech), into HEK-293 cells per the manufacturer's protocol. Cells were centrifuged, filtered, and the supernatants were purified using MabSelect Sure Resin (GE Healthcare, Chicago, Ill., USA). Purified molecules were analyzed using 4-12% Bis Tris SDS PAGE gels by loading 1-2 μg of each molecule into the gel, and staining using instant Blue staining. Each gel included a molecular weight ladder with the indicated molecular weight standards (FIGS. 1-7). Reduced and non-reduced lanes are denoted by “R” and “NR”.

### Example 2—Synthesis of Intermediate 1 (Int-1)

Degradation of Polymyxin B to Tri-Boc Polymyxin B Cycloheptapeptide

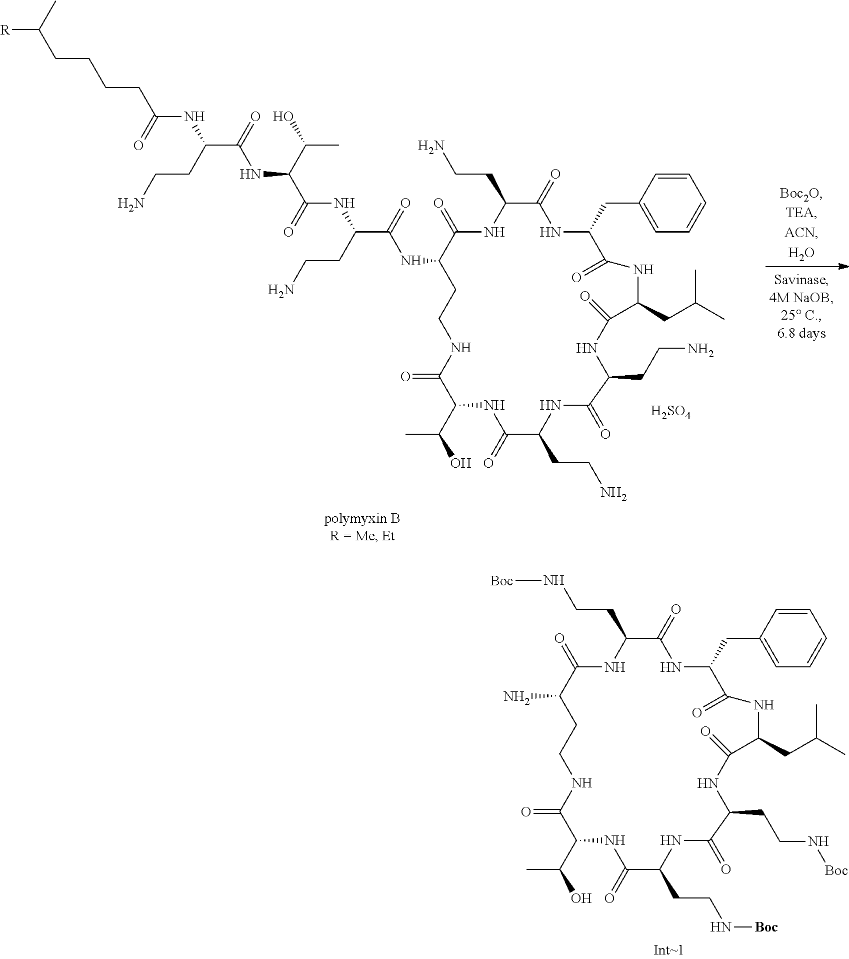

Polymyxin B (100 g, 72.2 mmol) was dissolved in acetonitrile (1000 mL) and water (500 mL) and stirred at room temperature for 10 mins. TEA (58.5 g, 8.0 eq) was added and the mixture stirred for a further 10 mins. Boc₂O (94.6 g, 6.0 eq) was subsequently added in one portion and the mixture stirred for 6 hrs at 20° C. Savinase® (300 mL) was then added. The pH of the resulting mixture was adjusted to 9.0 with aq 4M sodium hydroxide solution (10 mL) and the reaction mixture stirred at 25° C. Additional Savinase® (100 mL) was added after 17 hrs, and another quantity of Savinase® (100 mL×2) was added after 26 hrs. After an overall reaction time of 80 hrs, the mixture was diluted with ethyl acetate (2000 mL). After separation of the layers, the organic phase was washed with 0.1 M NaOH solution (1000 mL×2, 10V×2), then water (1000 mL, 10V). The organic layer was dried over anhydrous Na₂SO₄, filtered and the solvent evaporated at reduced pressure. The residue was purified by silica gel chromatography eluting with 80% (EtOAc:MeOH:H₂O.NH₃=40:10:1) in ethyl acetate to give the title compound (49.8 g, 65.0%). LCMS: m/z (M+H)⁺ calcd for C₅₀H₈₃N₁₁O₁₄:1061.61; found:1062.5

### Example 3—Synthesis of Int-2 (Tripeptide (Dab-Thr-Dab))

Step a.

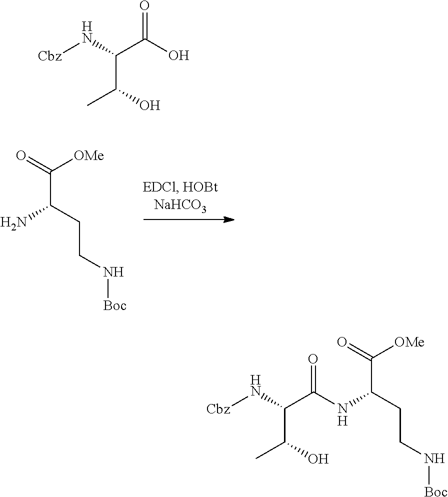 [SMILES: C[C@@H](O)[C@H](NC(=O)OCc1ccccc1)C(=O)O ; COC(=O)[C@@H](N)CCNC(=O)OC(C)(C)C ; "NaHCO3" ; COC(=O)[C@H](CCNC(=O)OC(C)(C)C)NC(=O)[C@@H](NC(=O)OCc1ccccc1)[C@@H](C)O]

NH₂-Dab(Boc)-OMe (HCl salt) (5.000 g, 1 eq.). Z-NH-Thr-OH (5.049 g, 1.05 eq.), EDCI (5.350 g, 1.5 eq.), HOBt (3.733 g, 1.5 eq.) and NaHCO₃ (3.095 g, 2 eq.) were weighed into a 100-mL round bottom flask. 24 mL of DCM/DMF (4:1) was added into the flask. The mixture was stirred at room temperature for less than 3 hrs. (TLC or LC/MS monitoring). After completion, EtOAc (200 mL) was added to dilute the reaction mixture and washed with 1N aq. HCl, saturated NaHCO₃ and brine. Dried with Na₂SO₄ and condensed. The residue was purified with normal phase silica (2˜7% MeOH/DCM) to give 8.24 g pure desired product (>95%).

Step b.

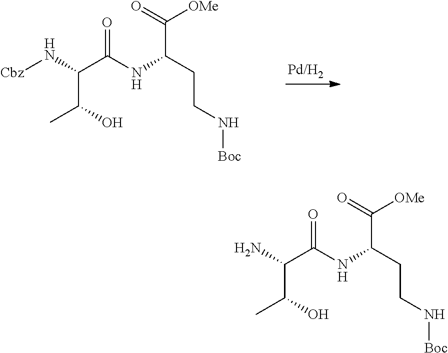 [SMILES: COC(=O)[C@H](CCNC(=O)OC(C)(C)C)NC(=O)[C@@H](NC(=O)OCc1ccccc1)[C@@H](C)O ; COC(=O)[C@H](CCNC(=O)OC(C)(C)C)NC(=O)[C@@H](N)[C@@H](C)O]

Starting material (SM) (8.24 g) was dissolved in 100 mL of MeOH/EtOAc. And 5% Pd/C (3.75 g, 0.1 eq.) was added. Under H₂ balloon, the reaction mixture was stirred for 2 h. Checked LC/MS. Celite filtration and MeOH wash. Dried to give 5.87 g of free amine (>99%). The material was used in the next step without purification.

Step c.

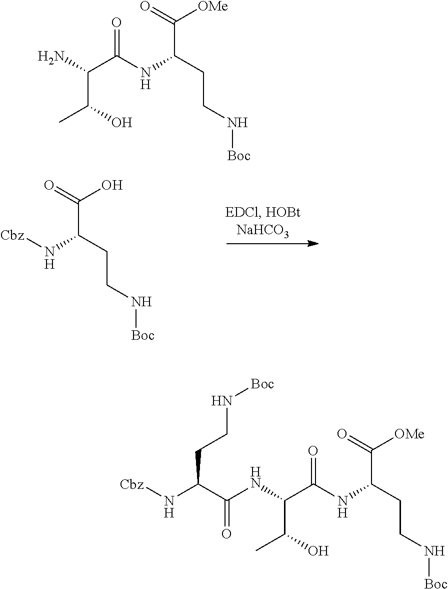 [SMILES: COC(=O)[C@H](CCNC(=O)OC(C)(C)C)NC(=O)[C@@H](N)[C@@H](C)O ; CC(C)(C)OC(=O)NCC[C@H](NC(=O)OCc1ccccc1)C(=O)O ; "NaHCO3" ; COC(=O)[C@H](CCNC(=O)OC(C)(C)C)NC(=O)[C@@H](NC(=O)[C@H](CCNC(=O)OC(C)(C)C)NC(=O)OCc1ccccc1)[C@@H](C)O]

NH₂-Thr-Dab(Boc)-OMe (1.46 g, 1 eq.), Z-NH-Dab(Boc)-OH(DCHA salt) (2.463, 1.05 eq.), EDCI (1.260 g, 1.5 eq.), HOBt (0.888 g, 1.5 eq.) and NaHCO₃ (0.738, 2 eq.) were weighed into a 100-mL round bottom flask. 20 mL of DCM/DMF (4:1) was added into the flask. The mixture was stirred at room temperature for less than 3 hrs with TLC or LC/MS monitoring. After the completion, EtOAc (100 mL) was added to dilute the reaction mixture and washed with 1N aq. HCl, saturated NaHCO₃ and brine. Dried with Na2SO4 and condensed. The residue was purified with normal phase silica (2˜7% MeOH/DCM) to give 2.78 g pure desired product (>95% isolated yield). (The reaction was repeated at 4.4 g scale and gave the similar result.)

Step d.

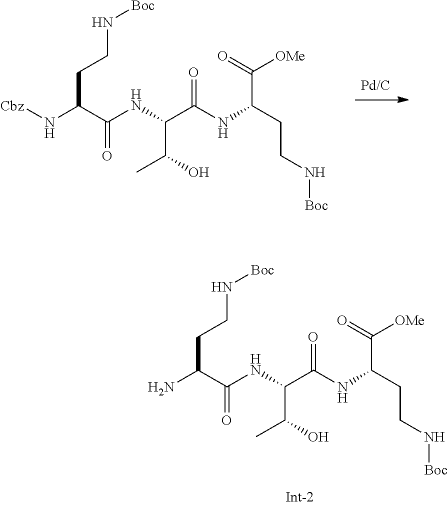 [SMILES: COC(=O)[C@H](CCNC(=O)OC(C)(C)C)NC(=O)[C@@H](NC(=O)[C@H](CCNC(=O)OC(C)(C)C)NC(=O)OCc1ccccc1)[C@@H](C)O ; COC(=O)[C@H](CCNC(=O)OC(C)(C)C)NC(=O)[C@@H](NC(=O)[C@@H](N)CCNC(=O)OC(C)(C)C)[C@@H](C)O]

A solution of the Cbz-tripeptide (1.55 g, 2.32 mmol) in methanol (10 mL) was charged with 5% Pd/C (0.300 g) and flushed with hydrogen from a balloon. After stirring overnight under hydrogen atmosphere, LCMS showed complete conversion. Pd/C was removed by filtration through Celite. The filtrate was concentrated and used in the next Step without further purification.

### Example 4—Synthesis of Int-3 (Tripeptide Acid)

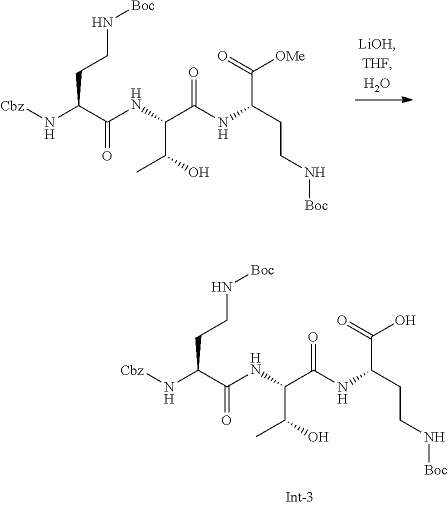 [SMILES: COC(=O)[C@H](CCNC(=O)OC(C)(C)C)NC(=O)[C@@H](NC(=O)[C@H](CCNC(=O)OC(C)(C)C)NC(=O)OCc1ccccc1)[C@@H](C)O ; C[C@@H](O)[C@H](NC(=O)[C@H](CCNC(=O)OC(C)(C)C)NC(=O)OCc1ccccc1)C(=O)N[C@@H](CCNC(=O)OC(C)(C)C)C(=O)O]

A solution of the Cbz-tripeptide-OMe (7.85 g, 11.76 mmol, described in Step 3 of Int-2 synthesis), dissolved in THF (30 mL), and water (30 mL), was treated with powdered LiOH (0.338 g, 14.1 mmol) and stirred at room temperature. After 15 minutes LCMS showed consumption of starting material. The reaction was made slightly acidic by adding concentrated HCl (aq), then extracted into ethyl acetate, dried over sodium sulfate, concentrated, and purified by RPLC using an Isco CombiFlash liquid chromatograph eluted with 5% to 100% methanol and water, using no modifier. Yield 7.2 g, 94%. Ion(s) found by LCMS: (M+H)+=654.2.

### Example 5—Synthesis of Int-4

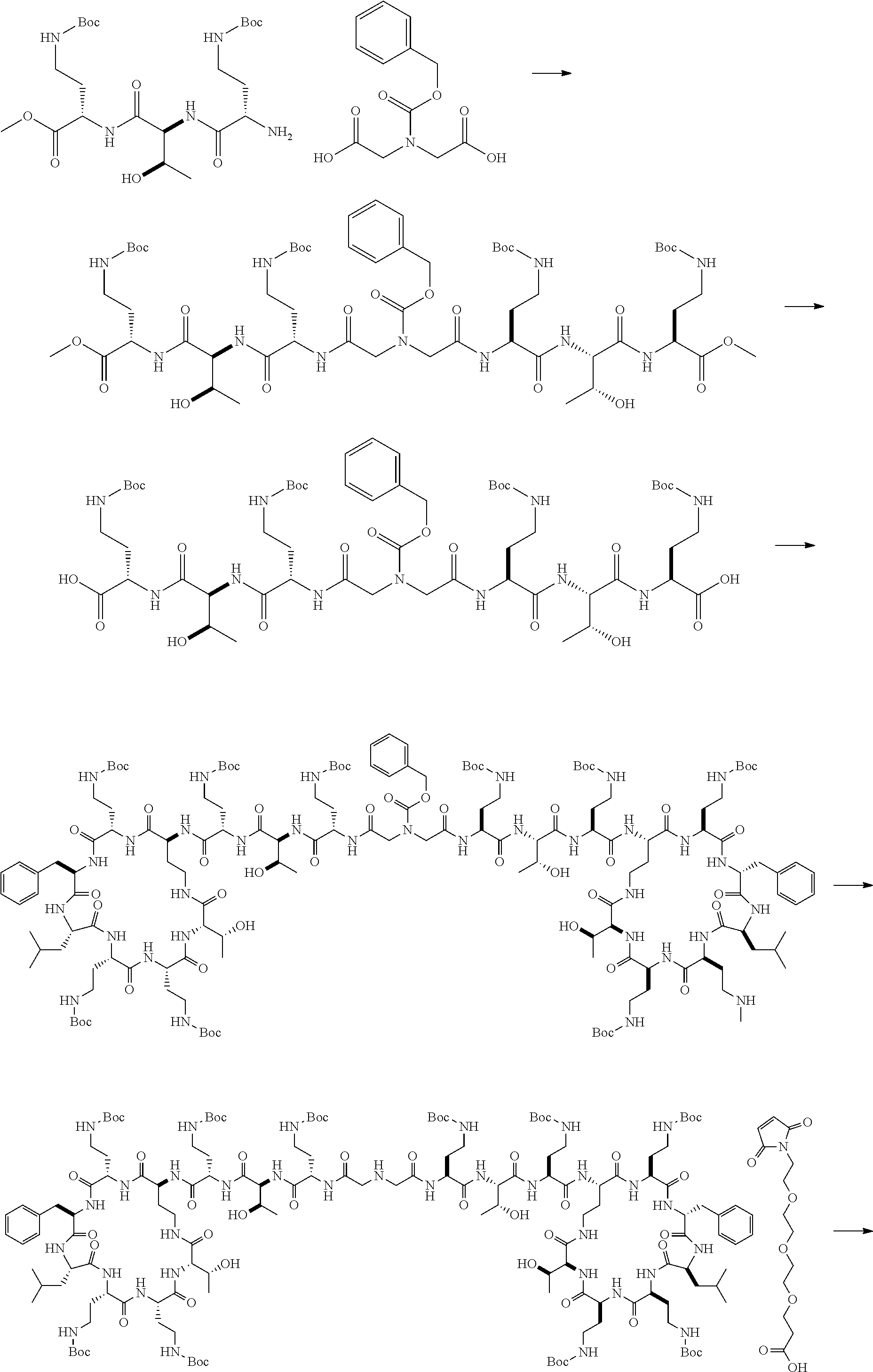

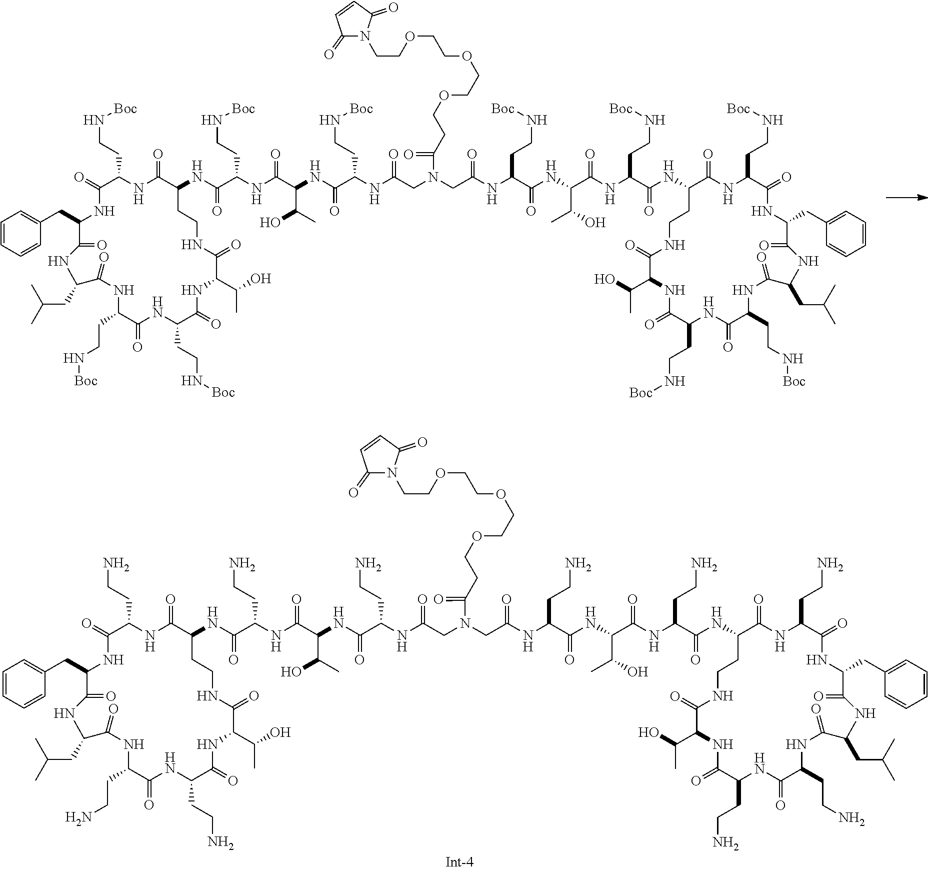

Step a.

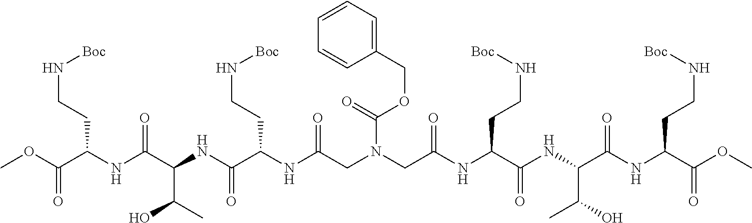

A stirring solution of methyl (2S)-2-[(N-{(2S)-2-amino-4-[(tert-butoxycarbonyl)amino]butanoyl}-L-threonyl)amino]-4-[(tert-butoxycarbonyl)amino]butanoate (0.399 g, 0.748 mmol), 2,2′-{[(benzyloxy)carbonyl]azanediyl}diacetic acid (0.100, 0.374 mmol), and DIEA (0.261 mL, 1.50 mmol), in DMF (1 mL), were treated with a solution of HATU (0.285 g, 0.748 mmol), dropwise over 30 minutes, at room temperature. The desired product was isolated by RPLC using an Isco CombiFlash liquid chromatograph eluted with 10% to 100% methanol and water, using no modifier. Yield 0.200 g, 41%. Ion(s) found by LCMS: (M+H)+=1298.7.

Step b.

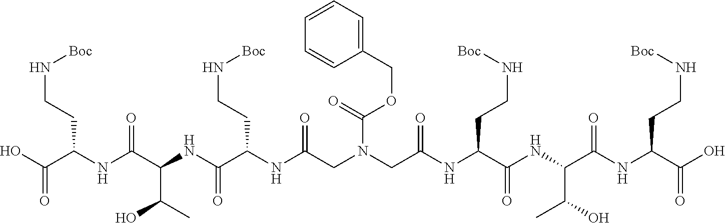 [SMILES: C[C@@H](O)[C@H](NC(=O)[C@H](CCNC(=O)OC(C)(C)C)NC(=O)CN(CC(=O)N[C@@H](CCNC(=O)OC(C)(C)C)C(=O)N[C@H](C(=O)N[C@@H](CCNC(=O)OC(C)(C)C)C(=O)O)[C@@H](C)O)C(=O)OCc1ccccc1)C(=O)N[C@@H](CCNC(=O)OC(C)(C)C)C(=O)O]

A solution of dimethyl (2S,5S,8S,16S,19S,22S)-12-[(benzyloxy)carbonyl]-2,8,16,22-tetrakis{2-[(tert-butoxycarbonyl)amino]ethyl}-5,19-bis[(1R)-1-hydroxyethyl]-4,7,10,14,17,20-hexaoxo-3,6,9,12,15,18,21-heptaazatricosane-1,23-dioate (0.380 g, 0.242 mmol), in methanol (1 mL) was treated with a solution of lithium hydroxide (0.028 g, 1.171 mmol), in water (1 mL), then stirred at room temperature for 30 minutes. The reaction was made slightly acidic (pH=5) with concentrated HCl (several drops). The desired product was isolated directly by RPLC using an Isco CombiFlash liquid chromatograph eluted with 10% to 100% methanol and water, using no modifier. Yield 0.164 g, 44%. Ion(s) found by LCMS: [(M−2Boc)/2]+H+=535.8, [(M−3Boc)/2]+H+=485.8

Step c

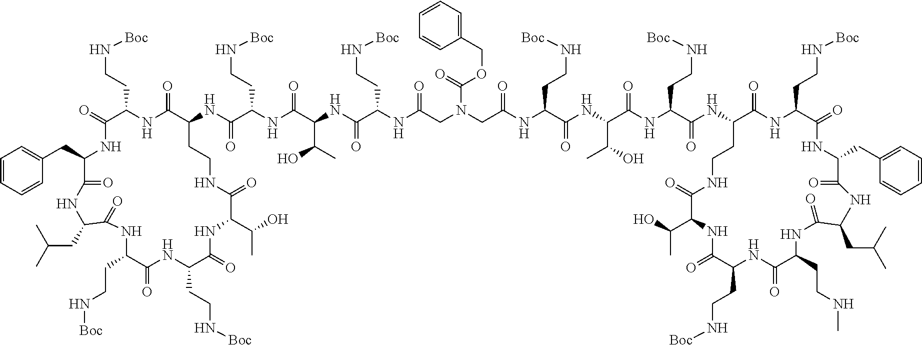

A solution of (2S,5S,8S,16S,19S,22S)-12-[(benzyloxy)carbonyl]-2,8,16,22-tetrakis{2-[(tert-butoxycarbonyl)amino]ethyl}-5,19-bis[(1R)-1-hydroxyethyl]-4,7,10,14,17,20-hexaoxo-3,6,9,12,15,18,21-heptaazatricosane-1,23-dioic acid (0.164 g, 0.129 mmol), tri-Boc polymyxin heptapeptide (0.329 g, 0.310 mmol, Int-1). DIEA (0.146 mL, 0.839 mmol), and DMF (1 mL), was treated with a solution of HATU (0.175 g, 0.460 mmol) in DMF (1 mL), dropwise over 30 minutes. The crude reaction mixture was taken on to the next Step without purification. Ion(s) found by LCMS: [(M−2Boc)/3]+H+=1053.3, [(M−3Boc)/3]+H+=1019.9. [(M−4Boc)/3]+H+=986.6.

Step d.

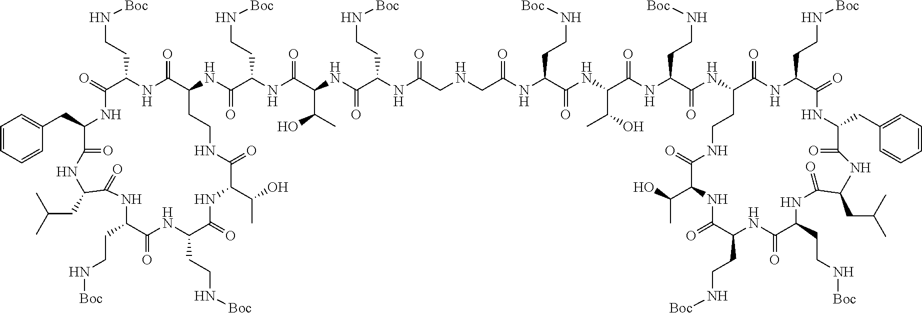

Crude Cbz deca Boc intermediate (DMF solution) was diluted with methanol (10 mL), charged with 5% Pd/C (0.150 g), and hydrogen from a balloon. The reaction was monitored by LCMS. After 2 hr the mixture was filtered through celite, concentrated and purified by RPLC using an Isco CombiFlash liquid chromatograph eluted with 10% to 100% methanol and water, using no modifier. Yield 0.252 g, 61% (two steps). Ion(s) found by LCMS: [M/3]+H+=1075.3.

Step e. Preparation of Deca Boc Int-4

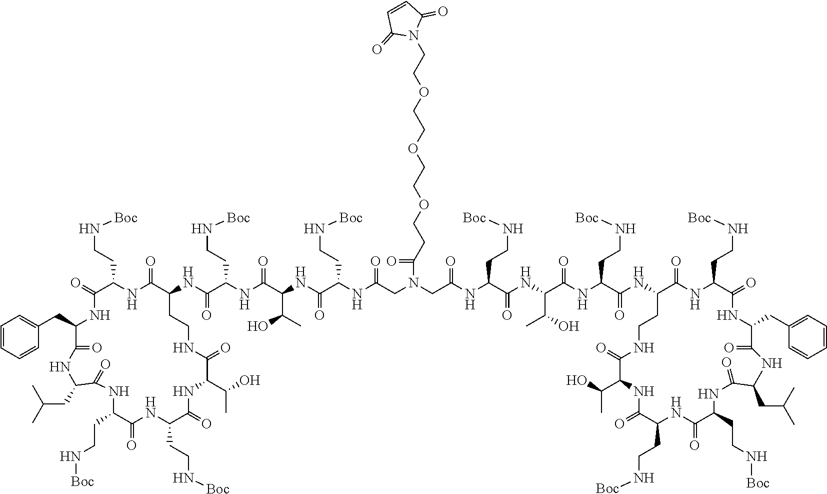

A solution of Step d product (0.135 g, 0.042 mmol), 3-(2-(2-[2-(2,5-dioxo-2,5-dihydro-1H-pyrrol-1-yl)ethoxy]ethoxy)ethoxy)propanoic acid (0.015 g, 0.050 mmol), and DIEA (0.026 mL, 0.150 mmol), in DMF (1 mL), were treated with HATU (0.019 g, 0.050 mmol), while stirring at room temperature. After 30 minutes, the product was purified by RPLC using an Isco CombiFlash liquid chromatograph eluted with 10% to 100% methanol and water, using no modifier. Yield 0.112 g, 76% yield. Ion(s) found by LCMS: [(M−3Boc)/3]+H+=1069.6, [(M−4Boc)/3]+H+=1036.3, [(M−5Boc)/3]+H+=1003.0.

Step f.

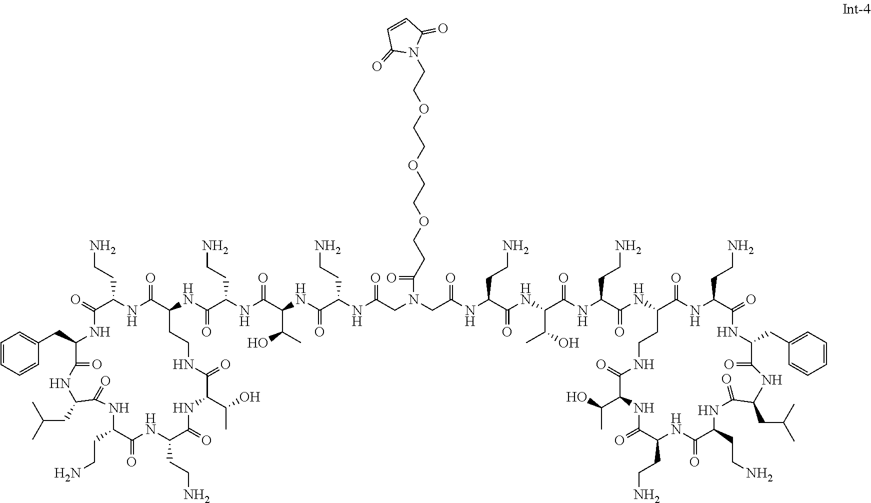 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)[C@H](CCN)NC(=O)CN(CC(=O)N[C@@H](CCN)C(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)C(=O)CCOCCOCCOCCN2C(=O)C=CC2=O)[C@@H](C)O)CCNC(=O)[C@H]([C@@H](C)O)NC(=O)[C@H](CCN)NC(=O)[C@H](CCN)NC1=O]

A solution of Deca Boc Int-4 (0.112 g, 0.032 mmol), dissolved in DCM (1 mL), was treated with TFA (1 mL), while stirring at room temperature. After 5 minutes, the product was concentrated and purified by RPLC using an Isco CombiFlash liquid chromatograph eluted with 0% to 100% acetonitrile and water, using trifluoroacetic acid as the modifier. Yield of Int-4 was 0.071 g, 88% yield. Ion(s) found by LCMS: [M/3]+H+=838.1, [M/4]+H+=627.3, [M/5]+H+=502.1.

### Example 6—Synthesis of Int-b

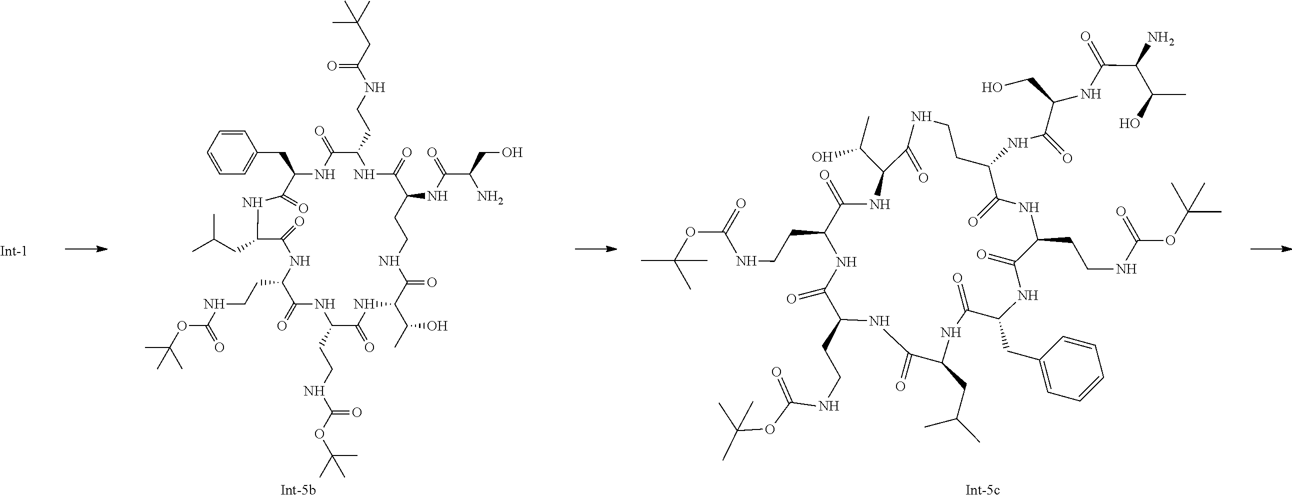 [SMILES: CC(C)C[C@@H]1NC(=O)[C@@H](Cc2ccccc2)NC(=O)[C@H](CCNC(=O)CC(C)(C)C)NC(=O)[C@@H](NC(=O)[C@H](N)CO)CCNC(=O)[C@H]([C@@H](C)O)NC(=O)[C@H](CCNC(=O)OC(C)(C)C)NC(=O)[C@H](CCNC(=O)OC(C)(C)C)NC1=O ; CC(C)C[C@@H]1NC(=O)[C@@H](Cc2ccccc2)NC(=O)[C@H](CCNC(=O)OC(C)(C)C)NC(=O)[C@@H](NC(=O)[C@@H](CO)NC(=O)[C@@H](N)[C@@H](C)O)CCNC(=O)[C@H]([C@@H](C)O)NC(=O)[C@H](CCNC(=O)OC(C)(C)C)NC(=O)[C@H](CCNC(=O)OC(C)(C)C)NC1=O]

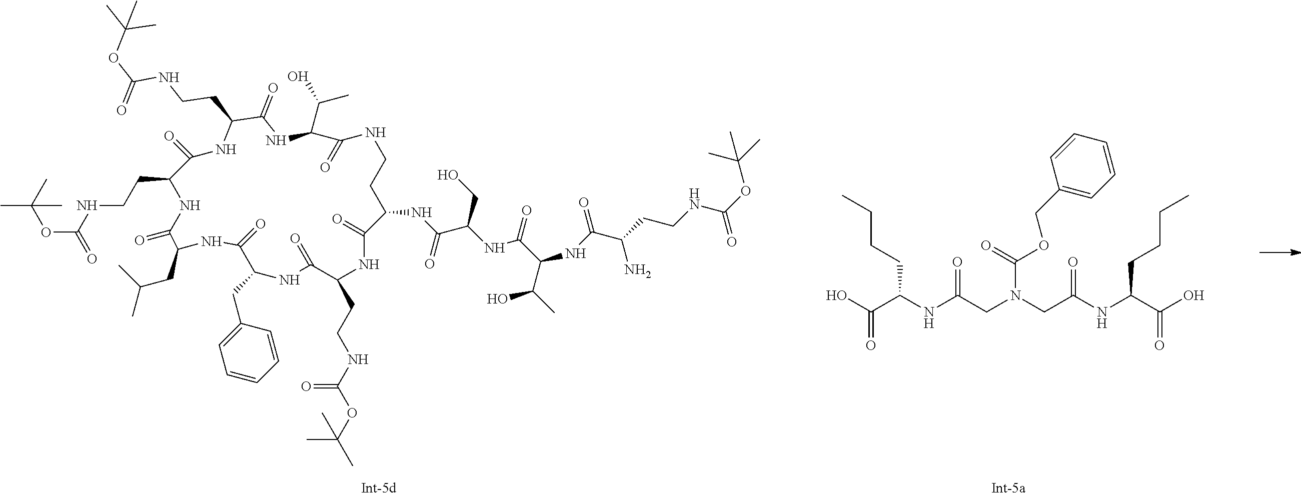 [SMILES: CC(C)C[C@@H]1NC(=O)[C@@H](Cc2ccccc2)NC(=O)[C@H](CCNC(=O)OC(C)(C)C)NC(=O)[C@@H](NC(=O)[C@@H](CO)NC(=O)[C@@H](NC(=O)[C@@H](N)CCNC(=O)OC(C)(C)C)[C@@H](C)O)CCNC(=O)[C@H]([C@@H](C)O)NC(=O)[C@H](CCNC(=O)OC(C)(C)C)NC(=O)[C@H](CCNC(=O)OC(C)(C)C)NC1=O ; CCCC[C@H](NC(=O)CN(CC(=O)N[C@@H](CCCC)C(=O)O)C(=O)OCc1ccccc1)C(=O)O]

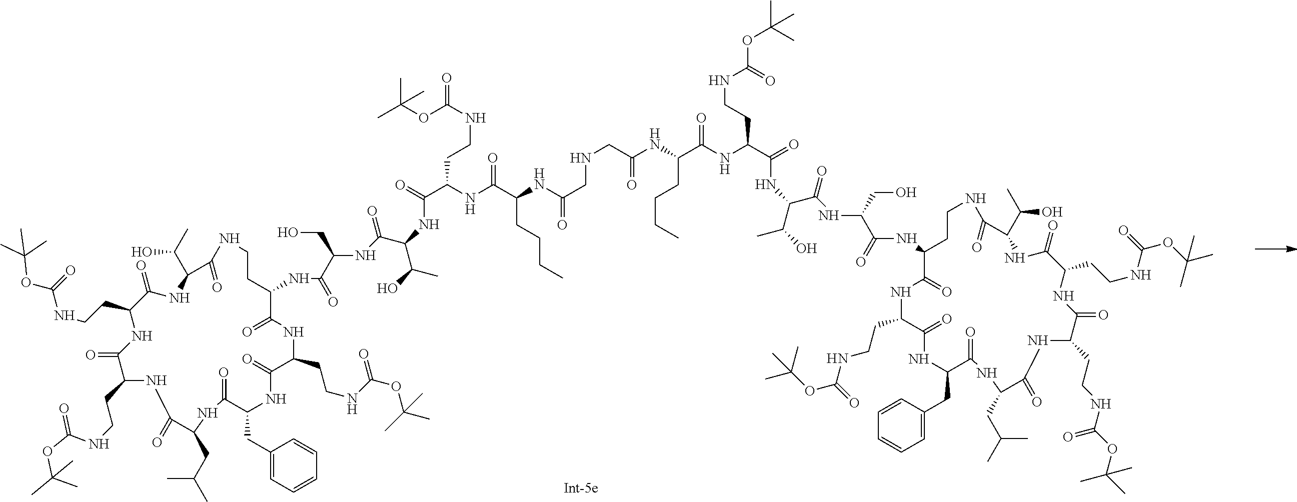

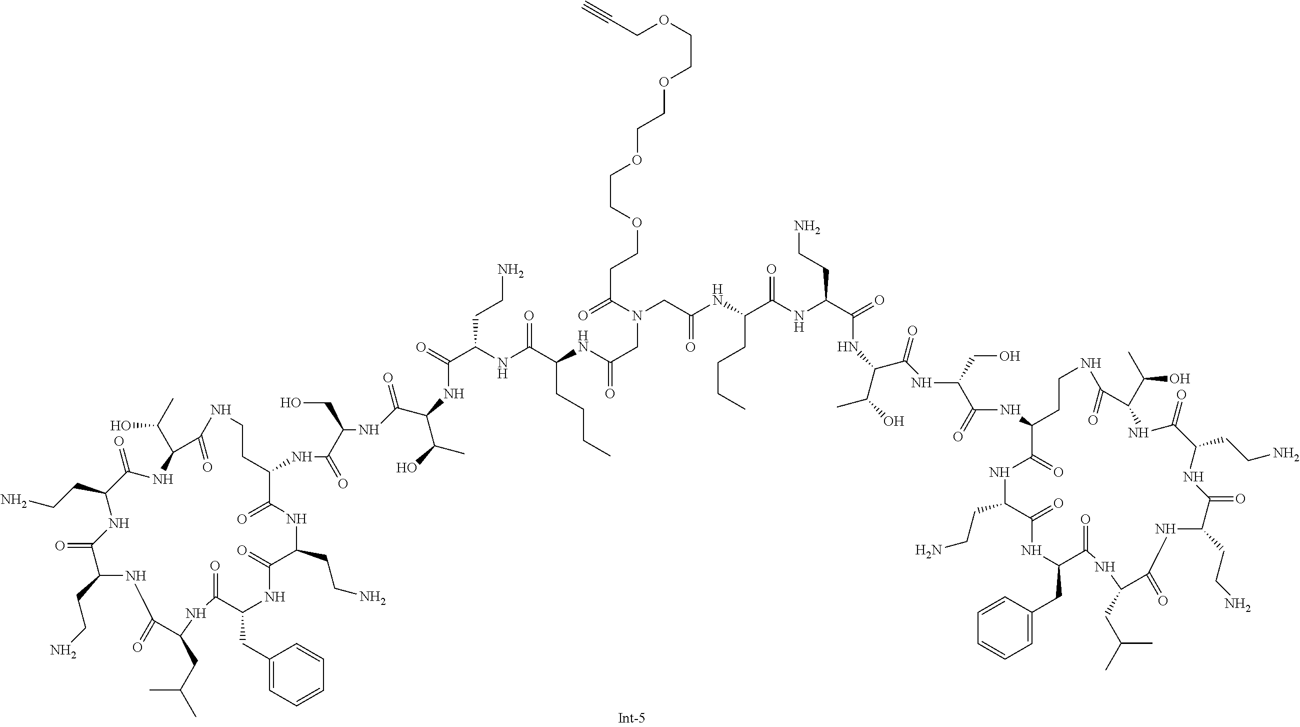 [SMILES: C#CCOCCOCCOCCOCCC(=O)N(CC(=O)N[C@@H](CCCC)C(=O)N[C@@H](CCN)C(=O)N[C@H](C(=O)N[C@H](CO)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O)CC(=O)N[C@@H](CCCC)C(=O)N[C@@H](CCN)C(=O)N[C@H](C(=O)N[C@H](CO)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O]

Step a. Synthesis of Int-5a.

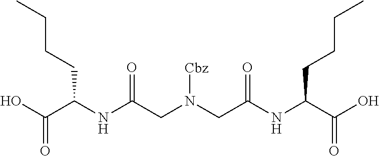 [SMILES: CCCC[C@H](NC(=O)CN(CC(=O)N[C@@H](CCCC)C(=O)O)C(=O)OCc1ccccc1)C(=O)O]

HATU (1.56 g, 4.11 mmol) in DMF (1.5 mL) was added, dropwise, to a solution of 2,2′-{[(benzyloxy)carbonyl]azanediyl}diacetic acid (0.5 g, 1.87 mmol), norleu-OMe hydrochloride salt (0.71 g, 3.93 mmol), and triethylamine (1.13 g, 11.23 mmol) in DMF (5 mL) over a period of 30 minutes. The mixture was stirred for an additional 20 minutes then applied directly to RPLC using an Isco Combiflash liquid chromatograph eluted with 10% to 95% acetonitrile and water using 0.1% TFA modifier. The pure fractions were pooled and lyophilized to afford the CBZ-protected di-ester as a clear oil, ion found by LC/MS [M+H]+=522.6. The di-ester intermediate was stirred in a 1/1/2 mixture (10 mL) of THF/methanol/water containing LiOH (0.18 g, 7.48 mmol) for 20 minutes. The mixture was applied directly to RPLC using an Isco Combiflash liquid chromatograph eluted with 10% to 95% acetonitrile and water using 0.1% TFA modifier. The pure fractions were pooled and lyophilized to afford (2S,2'S)-2,2′-({[(benzyloxy)carbonyl]azanediyl}bis[(1-oxoethane-2,1-diyl)azanediyl])dihexanoic acid as a white solid. Yield: 43%, 2 Steps. Ion found by LC/MS [m-H]−=492.3.

Step b. Synthesis of Int-6b.

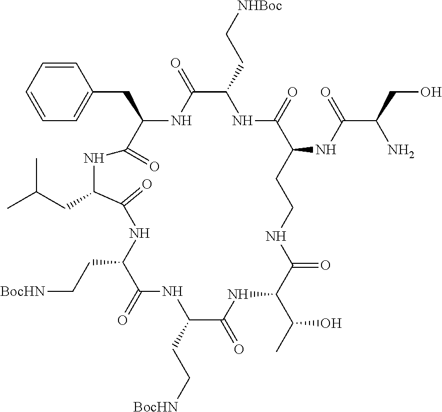 [SMILES: CC(C)C[C@@H]1NC(=O)[C@@H](Cc2ccccc2)NC(=O)[C@H](CCNC(=O)OC(C)(C)C)NC(=O)[C@@H](NC(=O)[C@H](N)CO)CCNC(=O)[C@H]([C@@H](C)O)NC(=O)[C@H](CCNC(=O)OC(C)(C)C)NC(=O)[C@H](CCNC(=O)OC(C)(C)C)NC1=O]

EDC (0.38 g, 2.0 mmol) was added to a solution of Int-1 (2.0 g, 1.88 mmol), Cbz-D-Ser-OH (0.48 g, 2.0 mmol), and HOBt (0.31 g, 2.0 mmol) in a 5/1 mixture of DCM/DMF (15 mL). The mixture was stirred for an additional 2 hours and applied directly to RPLC using an Isco Combiflash liquid chromatograph eluted with 10% to 95% acetonitrile and water using 0.1% TFA modifier. The pure fractions were pooled and lyophilized to afford the CBZ-protected intermediate a white solid. Ion found by LC/MS [m/2+H]+=542.4 (loss of 1 Boc group). The CBZ-protected intermediate was stirred in methanol (50 mL) containing 1 g of 5% Pd/C under 1 atmosphere of hydrogen for 6 hours. The mixture was filtered and concentrated to afford the Int-5b (free amine) as a white solid. Yield: 73%, 2 Steps. Ion found by LC/MS [m/2+H]+=525.6 (loss of 1 Boc group).

Step c. Synthesis of Int-5c.

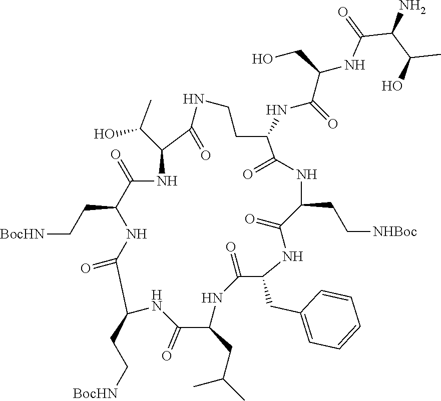 [SMILES: CC(C)C[C@@H]1NC(=O)[C@@H](Cc2ccccc2)NC(=O)[C@H](CCNC(=O)OC(C)(C)C)NC(=O)[C@@H](NC(=O)[C@@H](CO)NC(=O)[C@@H](N)[C@@H](C)O)CCNC(=O)[C@H]([C@@H](C)O)NC(=O)[C@H](CCNC(=O)OC(C)(C)C)NC(=O)[C@H](CCNC(=O)OC(C)(C)C)NC1=O]

HATU (370 mg, 0.97 mmol) in DMF (2 mL) was added, dropwise over 30 minutes, to a stirring solution of Int-5b (1.10 g, 0.95 mmol), CBZ-Thr-OH (253 mg, 1.0 mmol), and triethylamine (300 mg, 2.97 mmol) in DMF (6 mL). The reaction was stirred for 30 minutes and then applied directly to RPLC using an Isco Combiflash liquid chromatograph eluted with 10% to 95% acetonitrile and water using 0.1% TFA modifier. The pure fractions were pooled and lyophilized to afford 1.1 g of the CBZ-protected intermediate a white solid. LC/MS [m/2+H]⁺=593.0 (loss of 2 Boc groups). The CBZ-protected intermediate was stirred in methanol (50 mL) containing 0.30 g of 5% Pd/C under 1 atmosphere of hydrogen for 6 hours. The mixture was filtered and concentrated to afford the Int-5c (free amine) as a white solid. Yield: 66%, 2 Steps. Ion found by LC/MS [m/2+H]⁺=525.8 (loss of 2 Boc groups).

Step d. Synthesis of Int-5d.

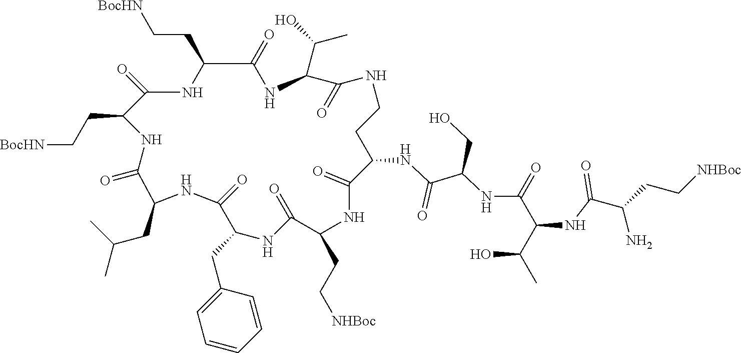 [SMILES: CC(C)C[C@@H]1NC(=O)[C@@H](Cc2ccccc2)NC(=O)[C@H](CCNC(=O)OC(C)(C)C)NC(=O)[C@@H](NC(=O)[C@@H](CO)NC(=O)[C@@H](NC(=O)[C@@H](N)CCNC(=O)OC(C)(C)C)[C@@H](C)O)CCNC(=O)[C@H]([C@@H](C)O)NC(=O)[C@H](CCNC(=O)OC(C)(C)C)NC(=O)[C@H](CCNC(=O)OC(C)(C)C)NC1=O]

The title compound was prepared from Int-5c and Z-(γBoc)-Dab-OH in an analogous manner as described in Step c. Yield: 78%. Ion found by LC/MS [M/2+H]⁺ 676.2 (loss of 1 Boc group).

Step e. Synthesis of Int-5e

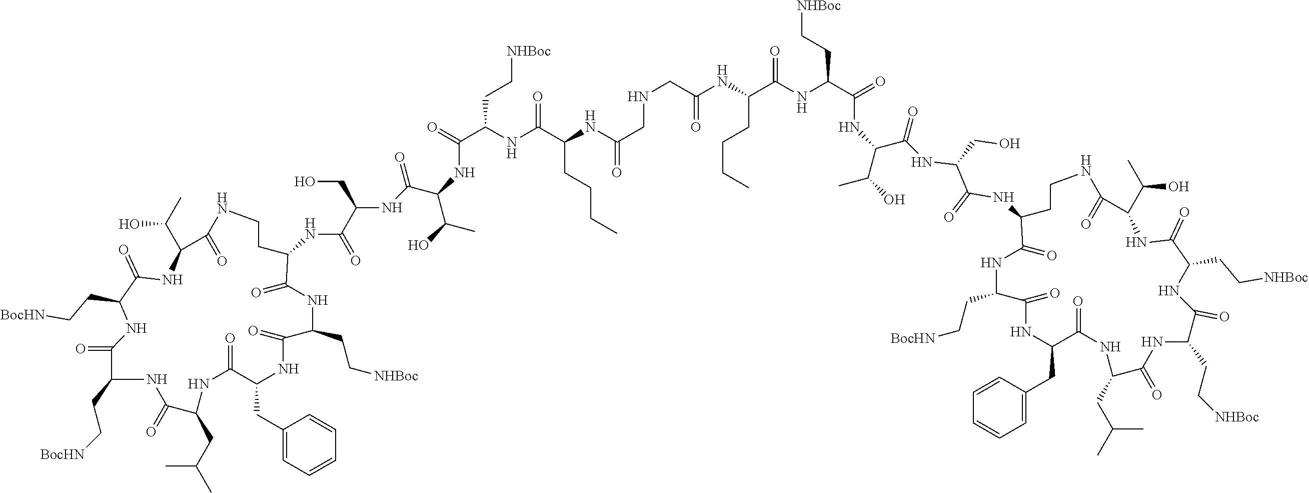

HATU (0.11 g, 0.30 mmol) in DMF (0.5 mL) was added, dropwise, to a stirring mixture of Int-5d (0.44 g, 0.30 mmol), and INT-A1 (0.70 g, 0.14 mmol) in DMF (1.5 mL) over a period of 20 minutes. The mixture was applied directly to RPLC using an Isco Combiflash liquid chromatograph eluted with 30% to 95% methanol and water using 0.1% TFA modifier. The pure fractions were pooled and lyophilized to afford the CBZ-protected intermediate, LC/MS [m/3+H]⁺ 1020.0 (loss of 3 Boc groups). The CBZ-intermediate was stirred in methanol (10 mL) in the presence of 5% Pd/C (100 mg) under 1 atmosphere of hydrogen for 2 hours. The mixture was filtered and concentrated then purified by RPLC using an Isco Combiflash liquid chromatograph eluted with 30% to 95% acetonitrile and water using no modifier. The pure fractions were pooled and lyophilized to afford the title compound as a white solid. Yield: 51%, 2 Steps. Ion found by LC/MS [m/2+H]⁺=1582.6 (loss of 2 Boc groups).

Step f. Synthesis of Int-5.

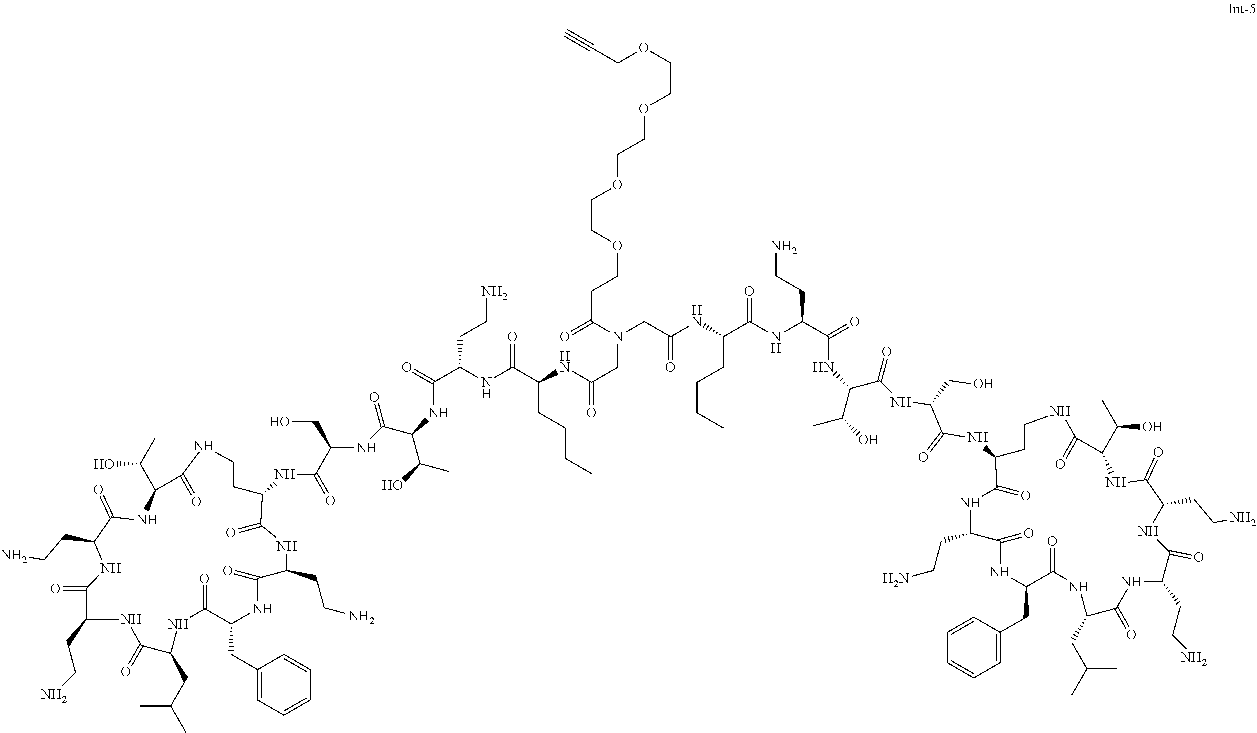 [SMILES: C#CCOCCOCCOCCOCCC(=O)N(CC(=O)N[C@@H](CCCC)C(=O)N[C@@H](CCN)C(=O)N[C@H](C(=O)N[C@H](CO)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O)CC(=O)N[C@@H](CCCC)C(=O)N[C@@H](CCN)C(=O)N[C@H](C(=O)N[C@H](CO)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O]

HATU (57 mg, 0.15 mmol) in DMF (1 mL) was added, dropwise, to a stirring mixture of propargyl-peg-4-carboxylic acid (42 mg, 0.16 mmol), and Int-5e (345 mg, 0.11 mmol) in DMF (1.5 mL) over a period of 20 minutes. The mixture was applied directly to RPLC using an Isco Combiflash liquid chromatograph eluted with 30% to 95% methanol and water using 0.1% TFA modifier. The pure fractions were pooled and lyophilized to afford the octa Boc-protected intermediate. The octa Boc-protected intermediate was stirred in a 1/1 mixture of TFA/DCM (5 mL) containing thioanisole (106 mg, 0.85 mmol) for 20 minutes at ambient temperature. The mixture was concentrated then purified by RPLC using an Isco Combiflash liquid chromatograph eluted with 0% to 70% acetonitrile and water using no modifier. The pure fractions were pooled and lyophilized to afford the title compound Int-5 as a white solid. Yield: 36%, 2 Steps. LC/MS [m/4+H]⁺=667.2.

### Example 7—Synthesis of Int-6 (Azido-Peg4-Hexyl Pyrolidine-3,4-Dicarboxamide)

### Step a. Synthesis of Int-6a (More Polar Diastereomer)

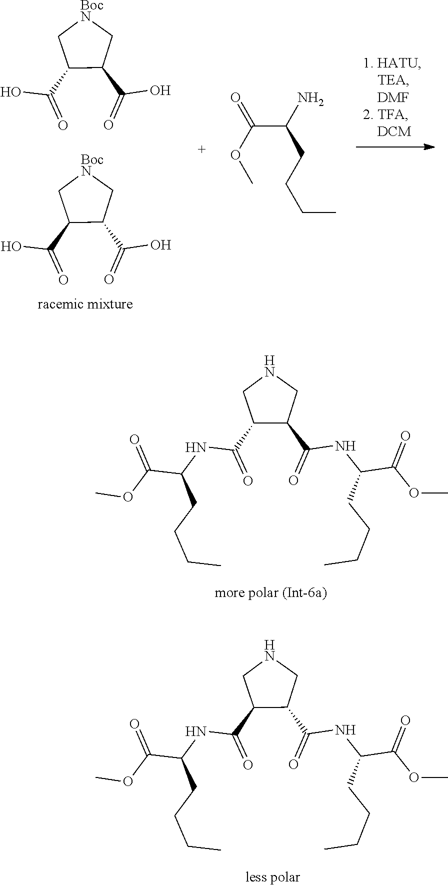

HATU (3.1 g, 8.1 mmol) in DMF (5 mL) was added, dropwise, to a solution of racemic-trans1-(tert-butoxycarbonyl)pyrrolidine-3,4-dicarboxylic acid (1 g, 3.9 mmol), H-Norleu-OMe-hydrochloride (1.5 g, 8.1 mmol), and triethylamine (4.0 g, 39.5 mmol) in DMF (10 mL) over a period of 20 minutes. The mixture was stirred for an additional 20 minutes then applied directly to RPLC using an Isco Combiflash liquid chromatograph eluted with 10% to 95% acetonitrile and water using 0.1% TFA modifier. The two diasteomers were separated, pooled and lyophilized separately into the more polar diasteomer (a) and the less polar diastereomer (b):Ion found by LC/MS [M+H]+=414.2 (loss of 1 Boc group) for both Boc-protected intermediates. Each diastereomer was stirred in a 1/1 mixture of DCM/TFA (8 mL) for 20 minutes then concentrated and purified by RPLC using an Isco Combiflash liquid chromatograph eluted with 0% to 75% acetonitrile and water using no modifier. Yield: 88%, 2 Steps. Ion found by LC/MS [M+H]+=414.2.

(The stereochemistry of the polar diastereomer was assigned using single crystal x-ray crystallography)

### Step b. Synthesis of Int-6

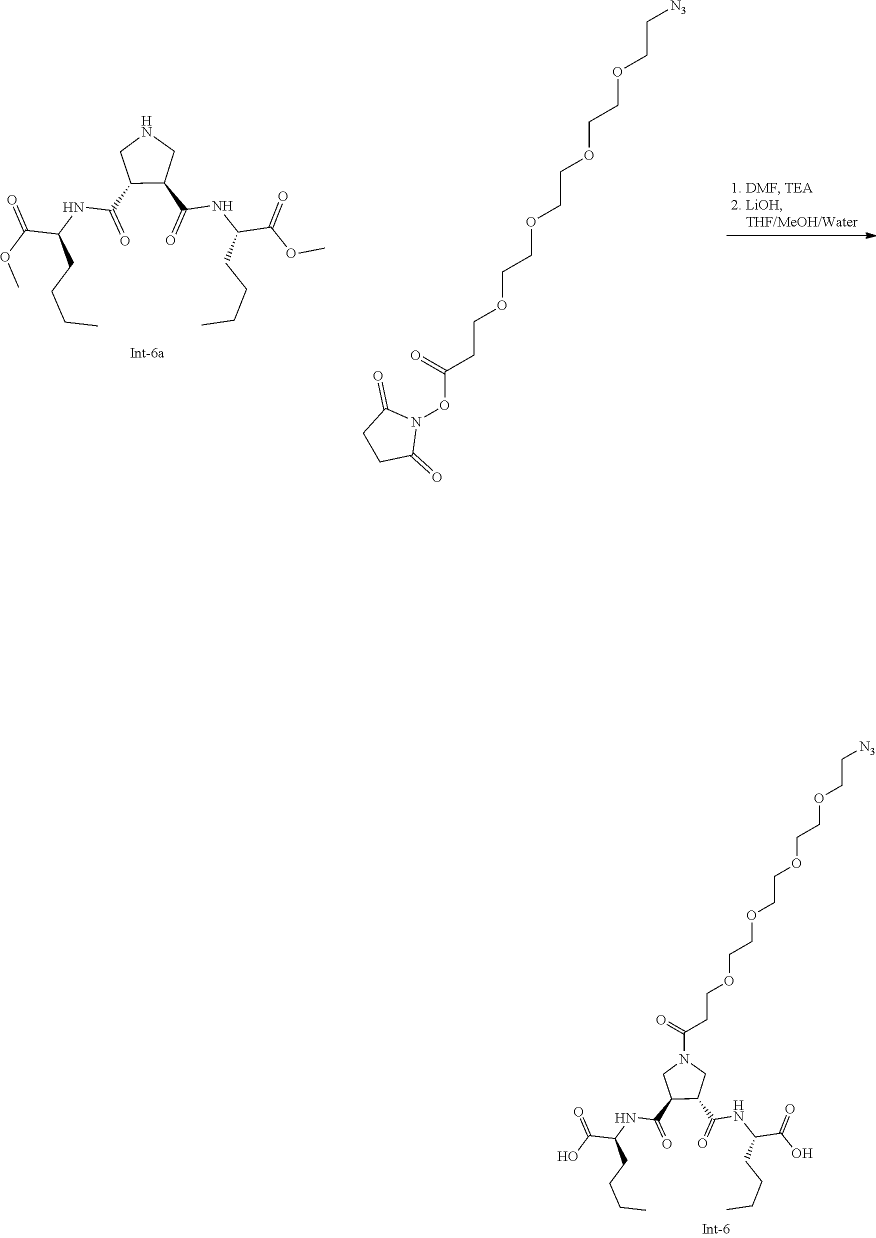 [SMILES: CCCC[C@H](NC(=O)[C@@H]1CNC[C@H]1C(=O)N[C@@H](CCCC)C(=O)OC)C(=O)OC ; [N-]=[N+]=NCCOCCOCCOCCOCCC(=O)ON1C(=O)CCC1=O ; CCCC[C@H](NC(=O)[C@H]1CN(C(=O)CCOCCOCCOCCOCCN=[N+]=[N-])C[C@@H]1C(=O)N[C@@H](CCCC)C(=O)O)C(=O)O]

Azido-PEG4-NHS ester (254 mg, 0.65 mmol) in DMF (1 mL) was added, dropwise to a mixture of Int-6a (more polar diastereomer from Step A), TFA salt (265 mg, 0.50 mmol) and triethylamine (66 mg, 0.65 mmol) in DMF (1 mL) and the mixture was stirred at ambient temperature for 2 hours. The mixture was applied directly to RPLC using an Isco Combiflash liquid chromatograph eluted with 10% to 95% acetonitrile and water using 0.1% TFA modifier. The pure fractions were pooled and lyophilized to afford the di-ester intermediate. LC/MS [M+H]⁺=687.4. The di-ester intermediate was stirred in a 1/1/2 mixture of MeOH/THF/DI water containing LiOH (36 mg, 1.5 mmol) at ambient temperature for 5 minutes. The mixture was acidified with glcial acetic acid, concentrated and applied directly to RPLC using an Isco Combiflash liquid chromatograph eluted with 10% to 95% acetonitrile and water using 0.1% TFA modifier. The pure fractions were pooled and lyophilized to afford the title compound Int-6 as a clear oil. Yield: 69%, 2 Steps. Ion found by LC/MS [M+H]⁺=659.4.

### Example—Synthesis of Int-7

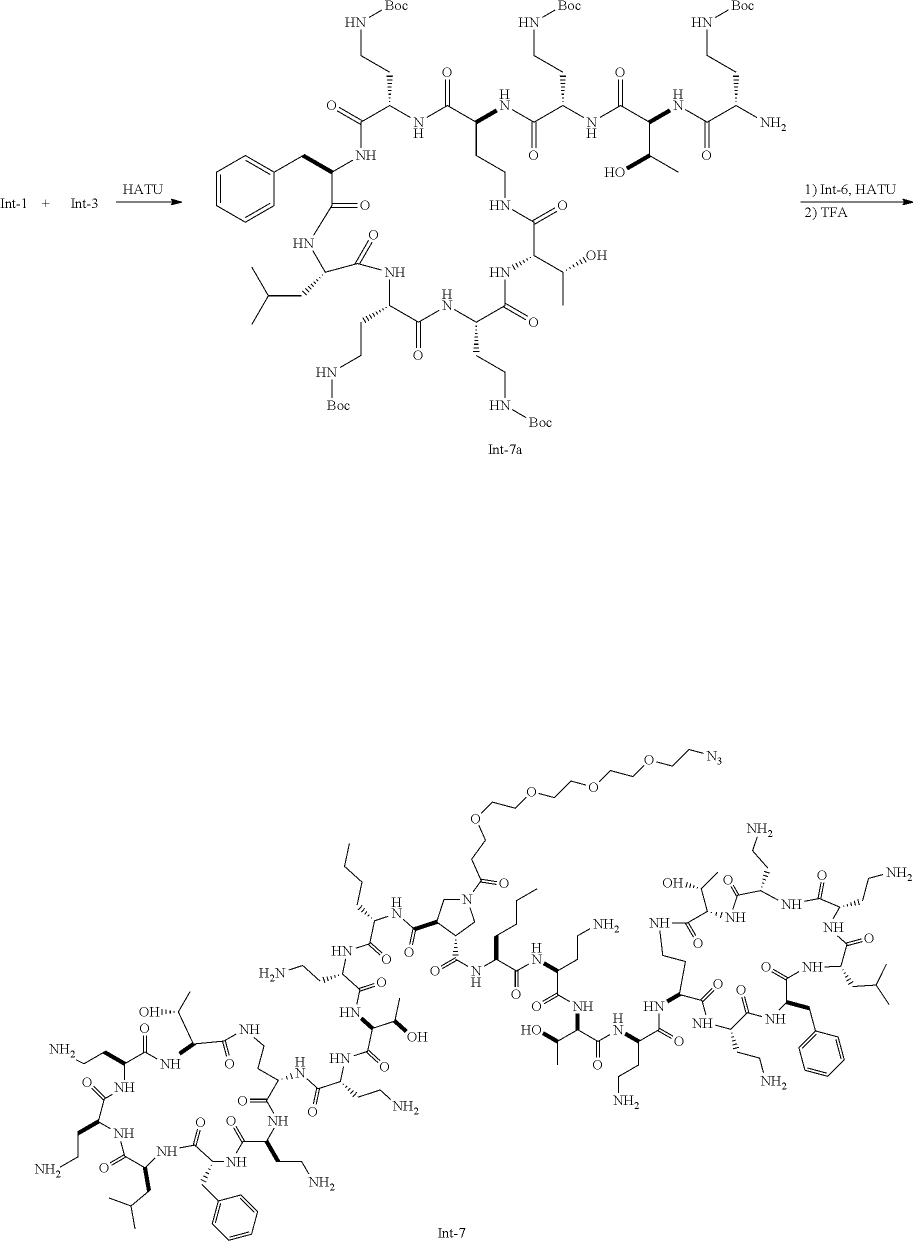

### Step a. Penta-Boc-PMB-Decapeptide (Int-7a)

Boc polymyxin B heptapeptide (0.464 g, 0.437 mmol, Int-1), and tripeptide acid (0.316 g, 0.398 mmol, Int-3), were dissolved in DMF (1 mL), DIEA (0.229 mL, 1.31 mmol), then treated with HATU (0.166 g, 0.437 mmol). Alter stirring for 30 min LCMS showed complete conversion. The crude Cbz-product was diluted with methanol (4 mL), and charged with 5% Pd/C (0.250 g) and placed under a hydrogen atmosphere. After 2 hr the mixture was filtered through Celite, concentrated, and purified by RPLC using an Isco CombiFlash liquid chromatograph eluted with 10% to 100% methanol and water, using no modifier. Yield 0.640 g, 94% (two steps). Ion(s) found by LCMS: [(M−1 Boc)/2]+H+=1054.6. [(M−2Boc)/2]+H+=1004.6.

### Step b. Coupling of Int-6 and Int-7a to Give Int-7

HATU (150 mg, 0.40 mmol) in DMF (1 mL) was added, dropwise, to a stirring mixture of Int-6 (120 mg, 0.30 mmol), and penta-Boc-PMB-decapeptide (626 mg, 0.40 mmol, Int-7a) in DMF (2 mL) over a period of 20 minutes. The mixture was applied directly to RPLC using an Isco Combiflash liquid chromatograph eluted with 30% to 95% methanol and water using 0.1% TFA modifier. The pure fractions were pooled and lyophilized to afford the Boc-protected intermediate, ion found by LC/MS [m/3+H]⁺1150.4 (loss of 3 Boc groups). The Boc-protected intermediate was stirred in a 1/1 mixture of TFA/DCM (6 mL) containing thioanisole (225 mg, 1.81 mmol) for 20 minutes at ambient temperature. The mixture was concentrated then purified by RPLC using an Isco Combiflash liquid chromatograph eluted with 0% to 70% acetonitrile and water using no modifier. The pure fractions were pooled and lyophilized to afford the title compound (Int-7) as a white solid. Yield: 68%, 2 Steps. Ion found by LC/MS [m/3+H]⁺=917.2.

### Example 9—Synthesis of Int-8

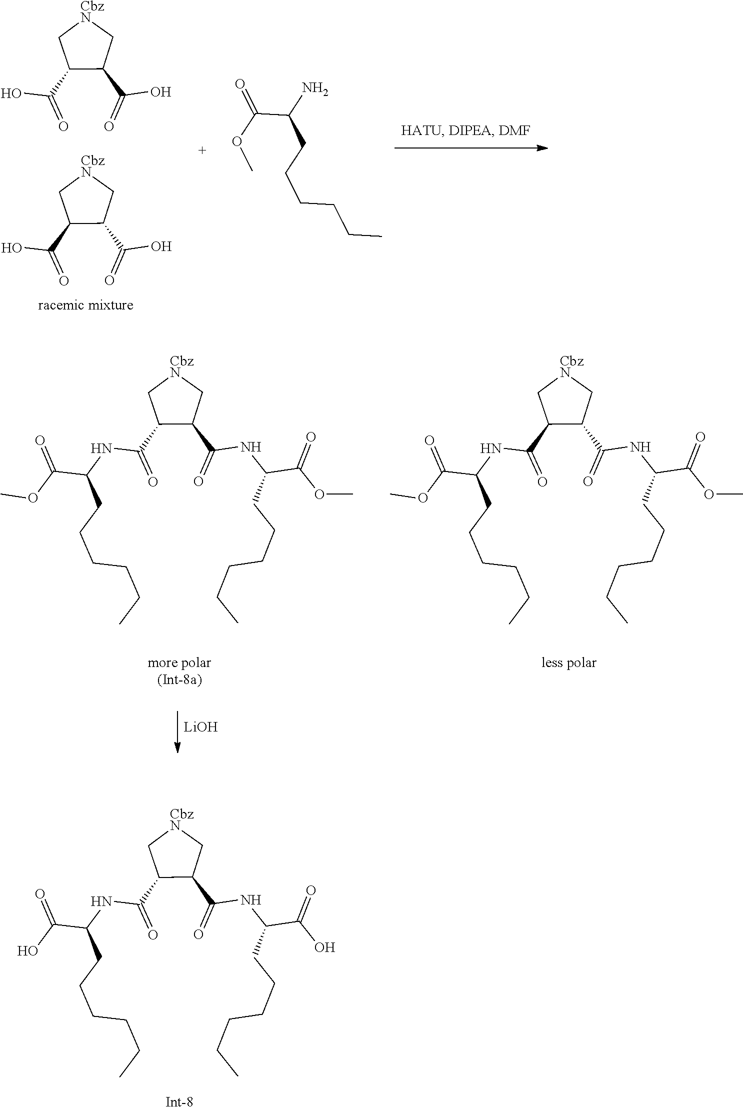

### Step a. Synthesis of benzyloxycarbonyl-3,4-bis{[(2S)-1-methoxy-1-oxooctan-2-yl]carbamoyl}pyrrolidine

A solution of racemic 1-[(benzyloxy)carbonyl]pyrrolidine-3,4-dicarboxylic acid (3.69 g, 12.6 mmol), methyl (2S)-2-aminooctanoate (4.58 g, 26.4 mmol), and DIEA (13.8 mL, 79.3 mmol), in DMF (20 mL) was treated with HATU (10.0 g, 26.4 mmol) at room temperature for 30 minutes. The crude product was purified by RPLC using an Isco CombiFlash liquid chromatograph eluted with 5% to 100% methanol and water, using no modifier. Two diastereomers where observed and were separable. The desired more polar isomer (Int-8a) had the shorter retention time and was taken on to Step b. Yield 1.44 g, 19%.

### Step b. Synthesis of Int-8

A solution of benzyloxycarbonyl-3,4-bis{[(2S)-1-methoxy-1-oxooctan-2-yl]carbamoyl}pyrrolidine (1.44 g, 2.38 mmol, more polar isomer from Step A, Int-8a) in THF (10 mL) and water (10 mL) was treated with powdered lithium hydroxide (0.143 g, 5.96 mmol) at room temperature. LCMS after 10 minutes showed complete conversion. The reaction was made slightly acidic by adding concentrated HCl, and concentrated to an oil. The oil was purified by RPLC using an Isco CombiFlash liquid chromatograph eluted with 5% to 100% acetonitrile and water, using TFA as the modifier. Yield 1.14 g, 83% yield.

### Example 10—Synthesis of Aminooctanoyl Penta Boc Polymyxin B Decapeptide (Int-18)

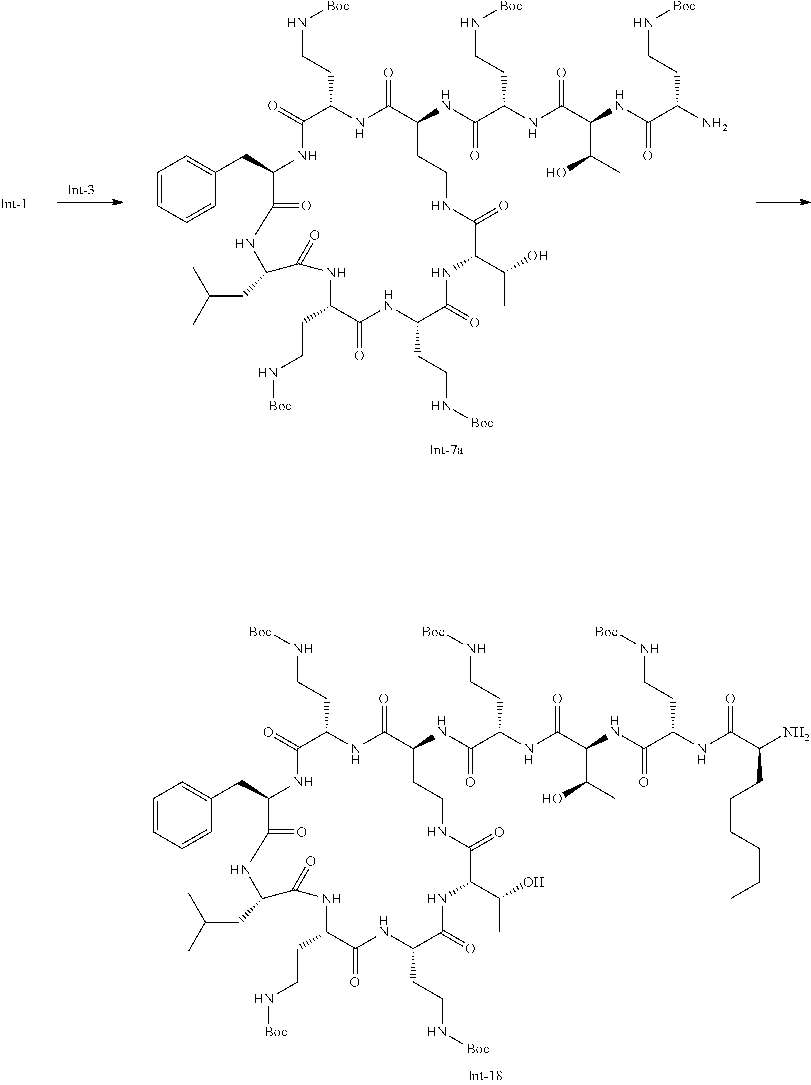

### Step a. Synthesis of Penta Boc Polymyxin B Decapeptide (Int-7a)

Tri-Boc polymyxin B heptapeptide (0.464, 0.437 mmol, Int-1), and tripeptide acid (0.316, 0.398 mmol, Int-3), were dissolved in DMF (1 mL), DIEA (0.229 mL, 1.31 mmol), then treated with HATU (0.166 g, 0.437 mmol). After stirring for 30 min LCMS showed complete conversion. The crude Cbz-product was diluted with methanol (4 mL), and charged with 5% Pd/C (0.250 g) and placed under a hydrogen atmosphere. After 2 hr the mixture was filtered through Celite, concentrated, and purified by RPLC using an Isco CombiFlash liquid chromatograph eluted with 10% to 100% methanol and water, using no modifier. Yield of Int-7a was 0.640 g, 94% (two steps). Ion(s) found by LCMS: [(M−1Boc)/2]+H+=1054.6, [(M−2Boc)/2]+H+=1004.6.

### Step b. Synthesis of Aminooctanoyl Penta Boc Polymyxin B Decapeptide (Int-18)

A solution of Int-7a (3.00 g, 1.918 mmol), Cbz-aminooctanoic acid (0.591 g, 2.01 mmol), and DIEA (1.05 mL, 6.04 mmol), in DMF (10 mL), was treated with a solution of HATU (0.766 g, 2.01 mmol) dissolved in DMF (3 mL), dropwise over 60 minutes. After 2 h, LCMS showed complete consumption of starting material. The crude mixture was treated with 5% Pd/C (1.7 g), vacuum flushed with hydrogen, and stirred under a hydrogen atmosphere for 2 hr or until complete by LCMS. The crude reaction was filtered through Celite, concentrated, and purified by RPLC using an Isco CombiFlash liquid chromatograph eluted with 20% to 100% methanol and water, using no modifier. Yield of Int-18 was 2.59 g, 79% (two steps). Ion(s) found by LCMS: [M/2]+H+=853.4.

### Example 11—Synthesis of Int-9

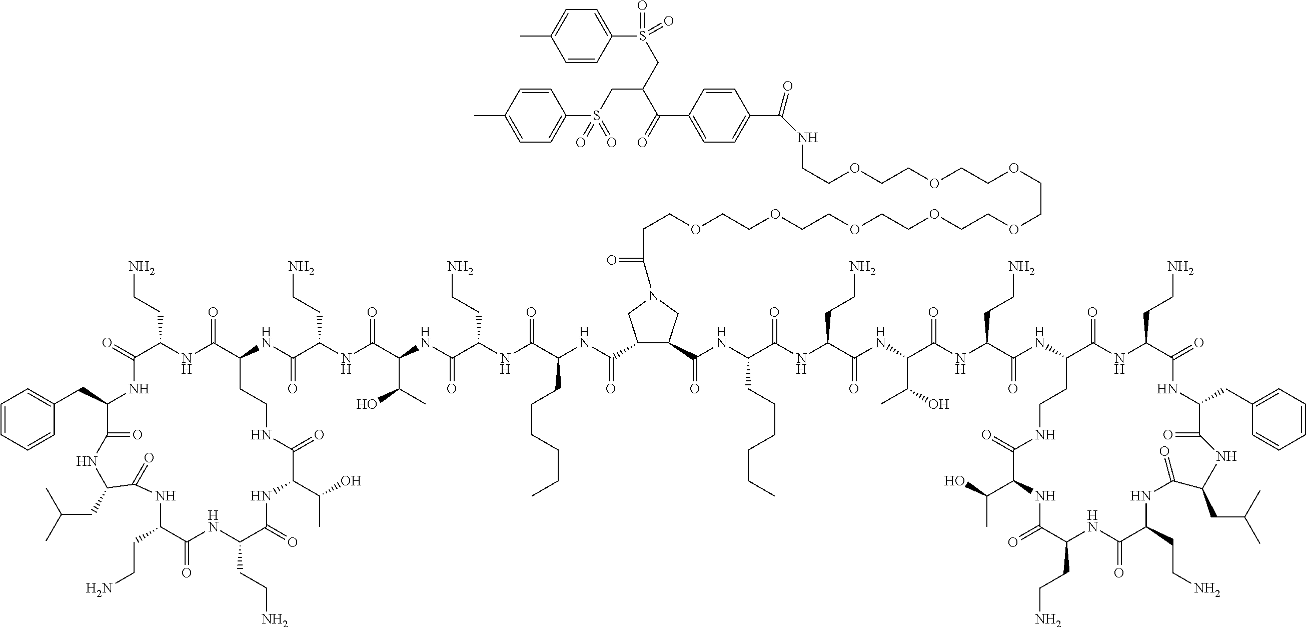

### Step a. Synthesis of Penta-Boc Dimer

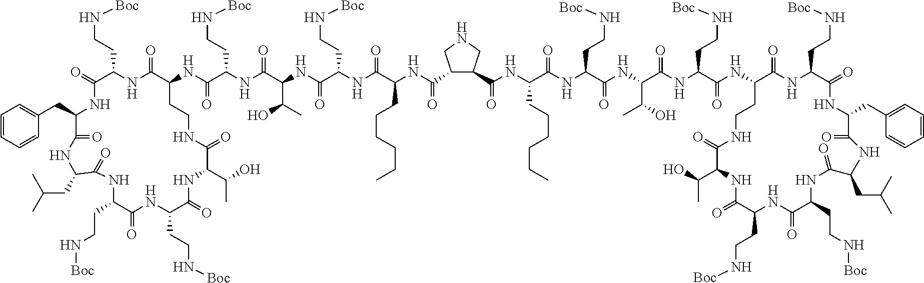

A solution of Int-8 (0.401 g, 0.70 mmol), Int-7a (2.18 g, 1.39 mmol), and DIEA (0.76 mL, 4.39 mmol) in DMF, was treated with a solution of HATU (0.557 g, 1.46 mmol) in DMF (5 mL), dropwise over 1 h. After stirring for an additional 30 minutes the reaction was charged with 5% Pd/C (1.5 g), vacuum flushed with hydrogen, and stirred under a hydrogen atmosphere for 2 h. The resulting mixture was filtered through celite, concentrated, and purified by RPLC using an Isco CombiFlash liquid chromatograph eluted with 30% to 100% methanol and water, using no modifier. Yield 1.39 g, 56% yield.

### Step b

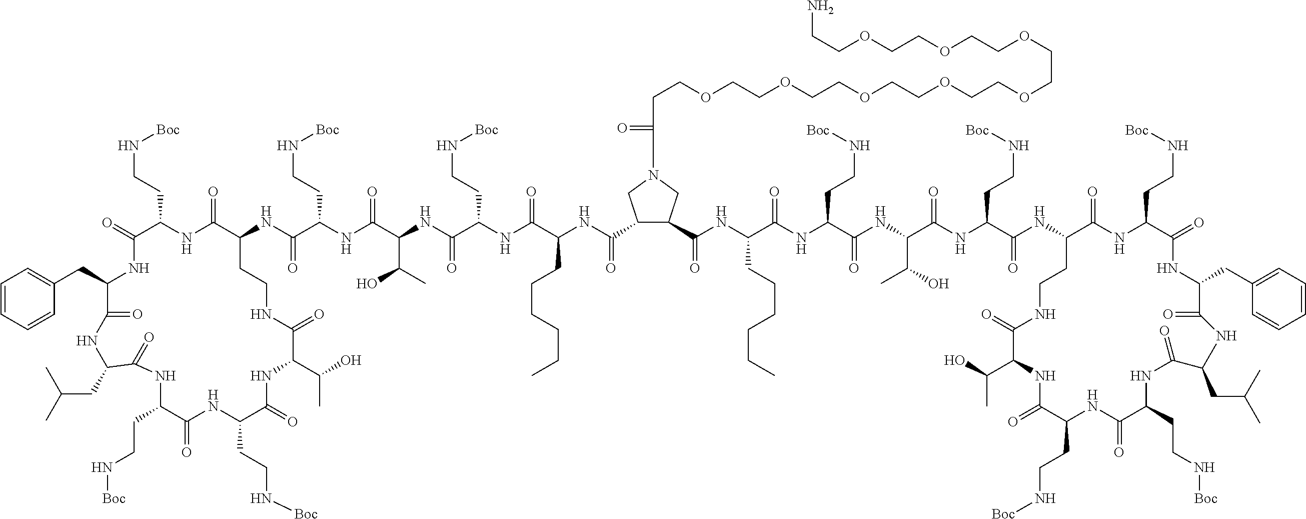

A solution of product from Step a (0.300 g, 0.085 mmol). Cbz-PEG8-acid (0.049 g, 0.085 mmol), and DIEA (0.049 mL, 0.280 mmol), in DMF (2 mL), was treated with a solution of HATU (0.036 g, 0.0934 mmol) in DMF (1 mL), dropwise over 1 h. After stirring an additional 30 minutes the reaction was charged with 5% Pd/C (0.35 g), vacuum flushed with hydrogen, and stirred under a hydrogen atmosphere for 2 h. The resulting mixture was filtered through celite, concentrated and purified by RPLC using an Isco CombiFlash liquid chromatograph eluted with 30% to 100% methanol and water, using no modifier. Yield 0.315 g, 94% yield. Ion(s) found by LCMS: [M−2(Boc)/3]+H+=1252.4.

### Step c

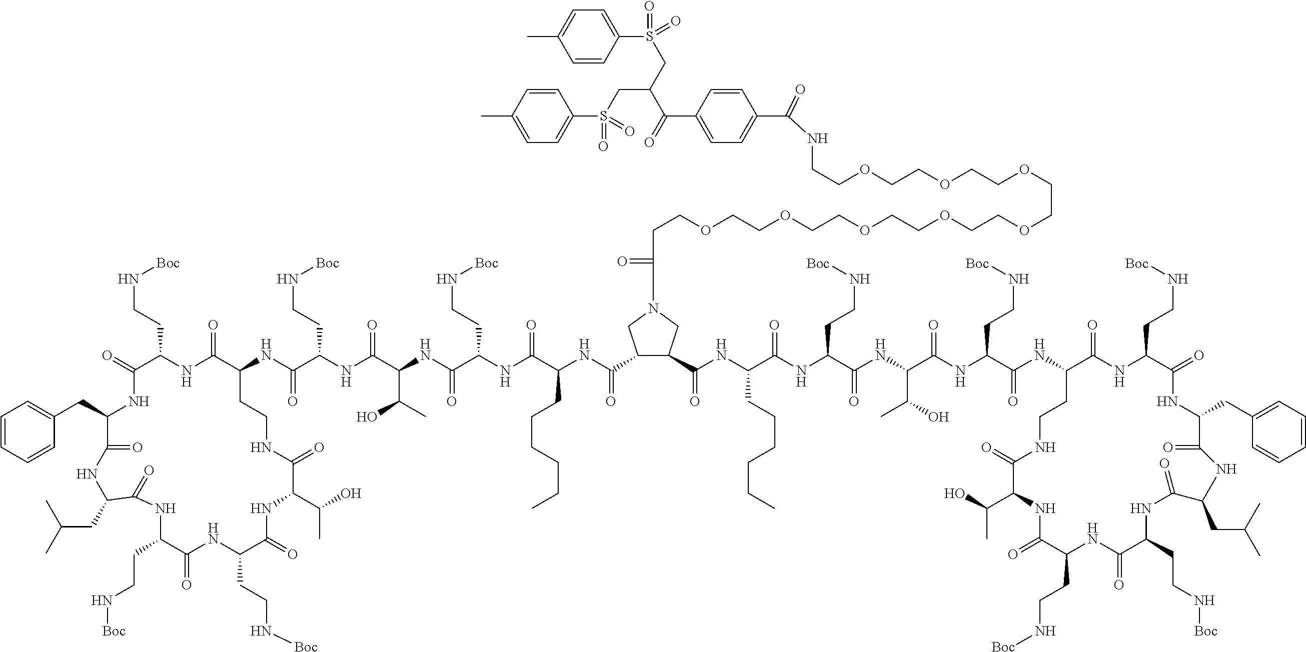

A solution of product from Step b (0.315 g, 0.080 mmol), bis-sulfone acid (0.044 g, 0.088 mmol), and N-methylmorpholine (0.020 mL, 0.175 mmol), in DMF (3 mL), was treated with a solution of HATU (0.033 g, 0.088 mmol), in DMF (1 mL), dropwise over 1 h. After stirring for an additional 30 minutes, the reaction was concentrated and purified by RPLC using an Isco CombiFlash liquid chromatograph eluted with 30% to 100% methanol and water, using no modifier. Yield 0.243 g, 69% yield. Ion(s) found by LCMS: did not ionize.

### Step d

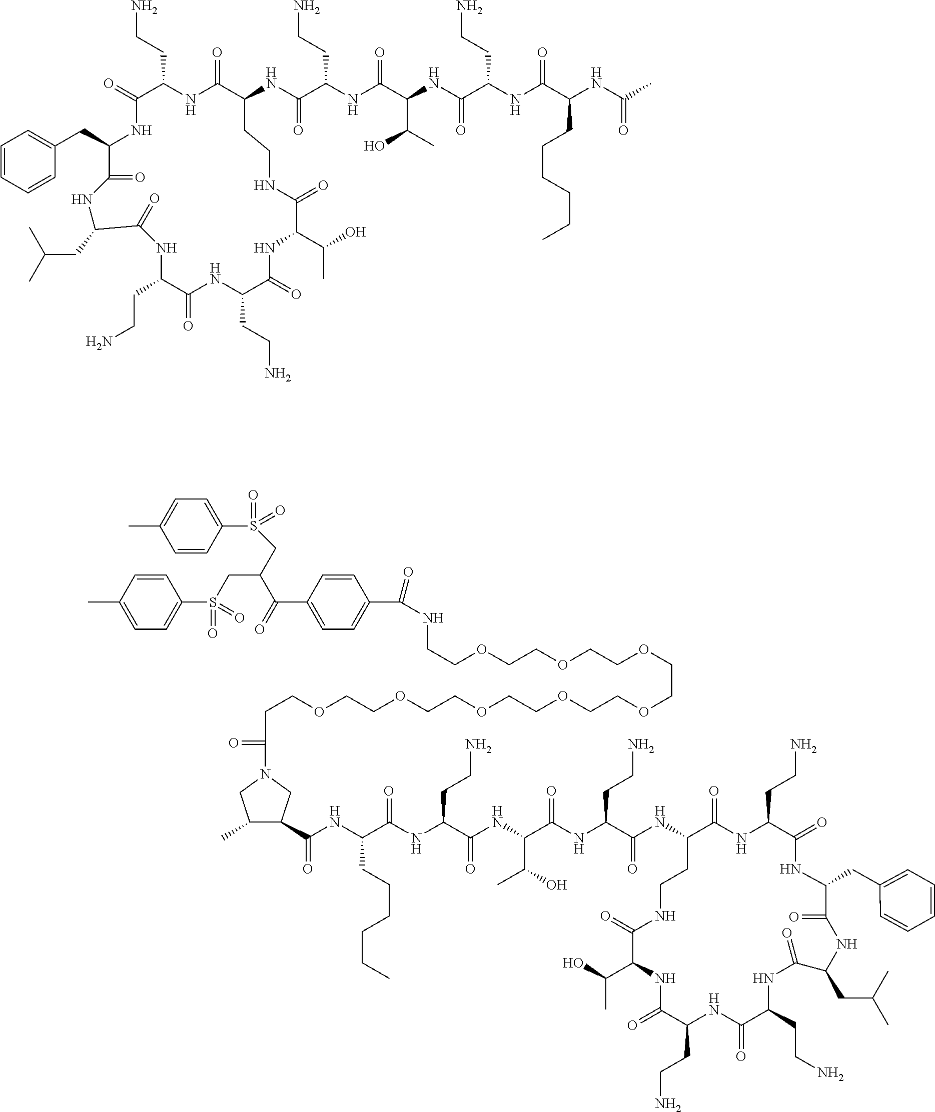

A solution of product from Step c (0.216 g, 0.049 mmol), suspended in DCM (2 mL) was treated with TFA (2 mL) at mom temperature. After stirring for 5 minutes, the reaction was concentrated and purified by RPLC using an Isco CombiFlash liquid chromatograph eluted with 5% to 100% acetonitrile and water, using TFA as the modifier. Yield of Int-9 was 0.226 g, quantitative yield. Ion(s) found by LCMS: [M/3]+H+=1146.3, [M/4]+H+=860.0, [M/5]+H+=688.2.

### Example 12—Synthesis of Int-10

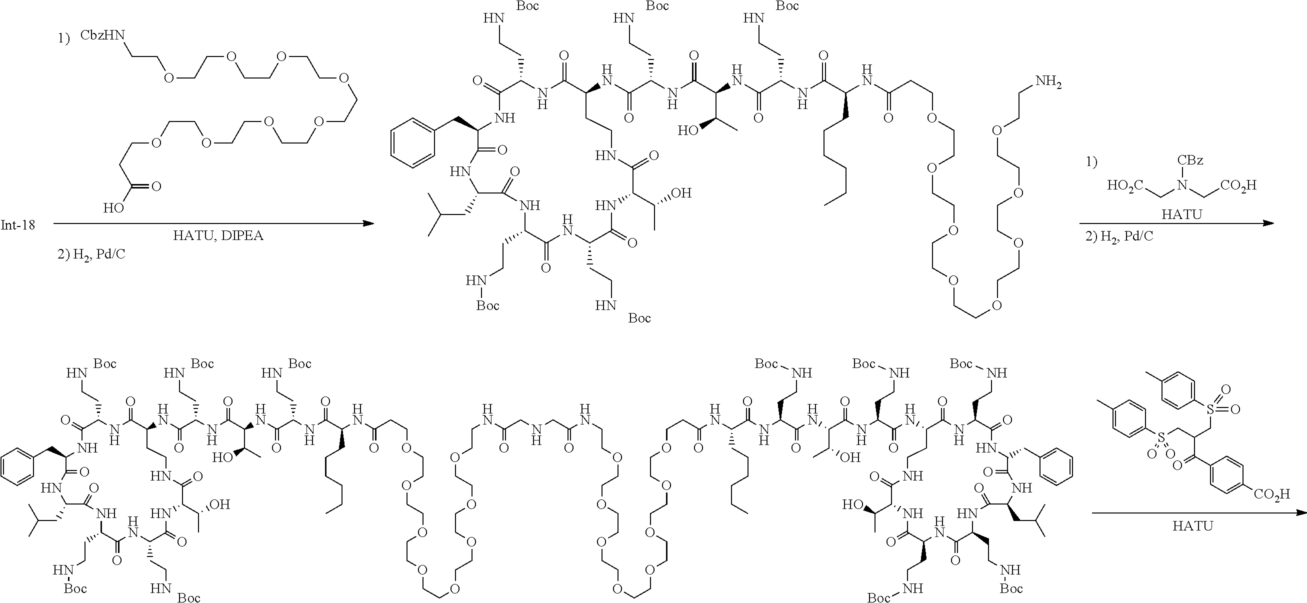

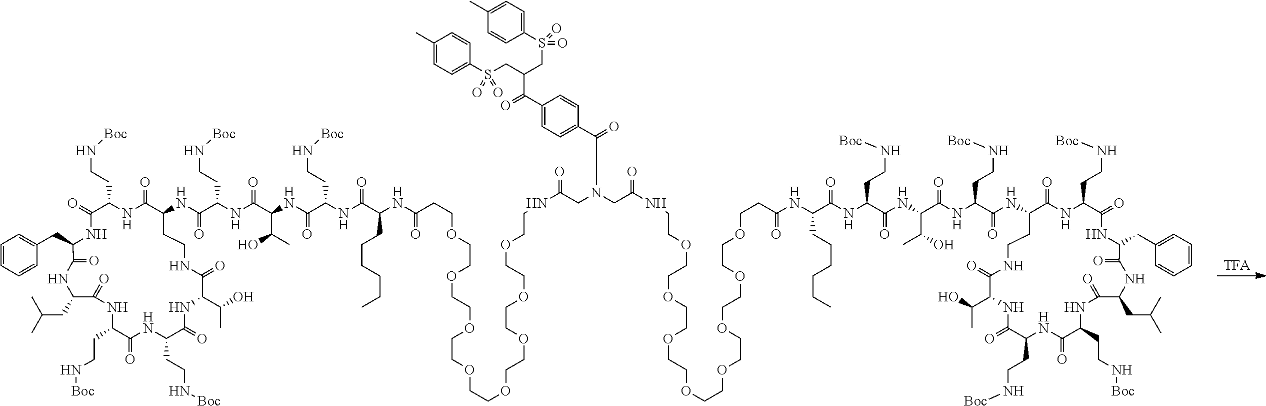

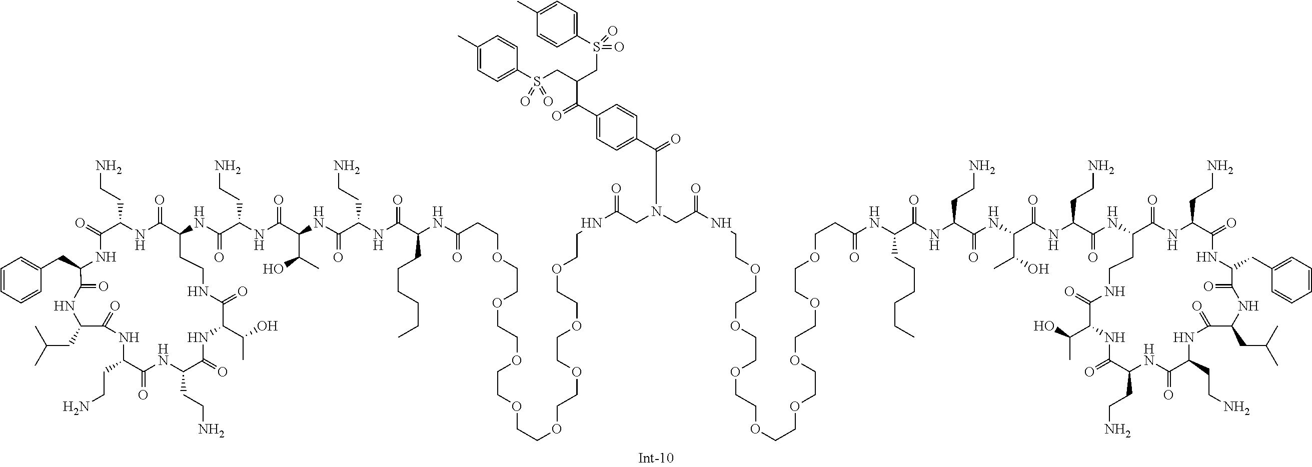

### Step a. Synthesis of Amino Peg 8 Penta Boc Polymyxin B Undecapeptide

A solution of Int-8 (0.600 g, 0.352 mmol), Cbz-amino Peg 8 acid (0.223 g, 0.387 mmol), and DIEA (0.202 mL, 1.161 mmol), in DMF (3 mL) was treated with a solution of HATU (0.147 g, 0.387 mmol), in DMF (1 mL), dropwise over 1 hr. After stirring for an additional 30 minutes, the reaction was charged with 5% Pd/C (0.400 g), vacuum flushed with hydrogen, and stirred under a hydrogen atmosphere until complete by LCMS (2 h). The resulting mixture was filtered through celite, concentrated, and purified by RPLC using an Isco CombiFlash liquid chromatograph eluted with 30% to 100% methanol and water, using no modifier. Yield 0.450 g, 64%. Ion(s) found by LCMS: [M/2]+H+=1064.6, [M−1(Boc)/2]+H+=1014.6.

### Step b. Synthesis of Deca Boc PEG8 Polymyxin B Dimer

A solution of amino PEG8 penta Boc polymyxin B undecapeptide (0.74 g, 0.350 mmol), Cbz iminodiacetic acid (0.044 g, 0.171 mmol), and DIEA (0.197 mL, 1.13 mmol), in DMF (5 mL), was treated with a solution of HATU (0.133 g, 0.350 mmol) in DMF (2 mL), dropwise over 1 h. After stirring an additional 30 minutes, the reaction was charged with 5% Pd/C (0.350 g), vacuum flushed with hydrogen, and stirred under a hydrogen atmosphere until complete by LCMS. The crude reaction was purified by RPLC using an Isco CombiFlash liquid chromatograph eluted with 30% to 100% methanol and water, using no modifier. Yield 0.465 g, 62% (two steps). Ion(s) found by LCMS: [M−3(Boc)/4]+H+=897.3, [M−4(Boc)/4]+H+=872.3.

### Step c. Synthesis of Deca Boc PEG 8 Polymyxin B Dimer Bis Sulfone

A solution of deca Boc PEG 8 polymyxin B dimer (0.465 g, 0.107 mmol), bis sulfone acid (0.059 g, 0.117 mmol), and N-methylmorpholine (0.026 mL, 0.235 mmol), in DMF (5 mL) was treated with a solution of HATU (0.045 g, 0.117 mmol) and DMF (1 mL), dropwise over 1 h. The resulting solution was stirred for an additional 30 minutes, then concentrated to a viscous oil. The crude oil was used in the next Step without further purification. Yield 0.465 g, 62% (two steps). Ion(s) found by LCMS: did not ionize.

### Step d. Synthesis of Int-10

A solution of crude deca Boc PEG 8 polymyxin B dimer bis sulfone, suspended in DCM (4 mL), was treated with TFA (4 mL), and stirred at room temperature for 5 min. The resulting solution was concentrated, and purified by RPLC using an Isco CombiFlash liquid chromatograph eluted with 5% to 100% methanol and water, using TFA as the modifier. Int-10 was obtained in a yield of 0.099 g, 27% (three steps). Ion(s) found by LCMS: [M/2]+H+=1917.5, [M/3]+H+=1278.7.

### Example 13—Synthesis of Int-11

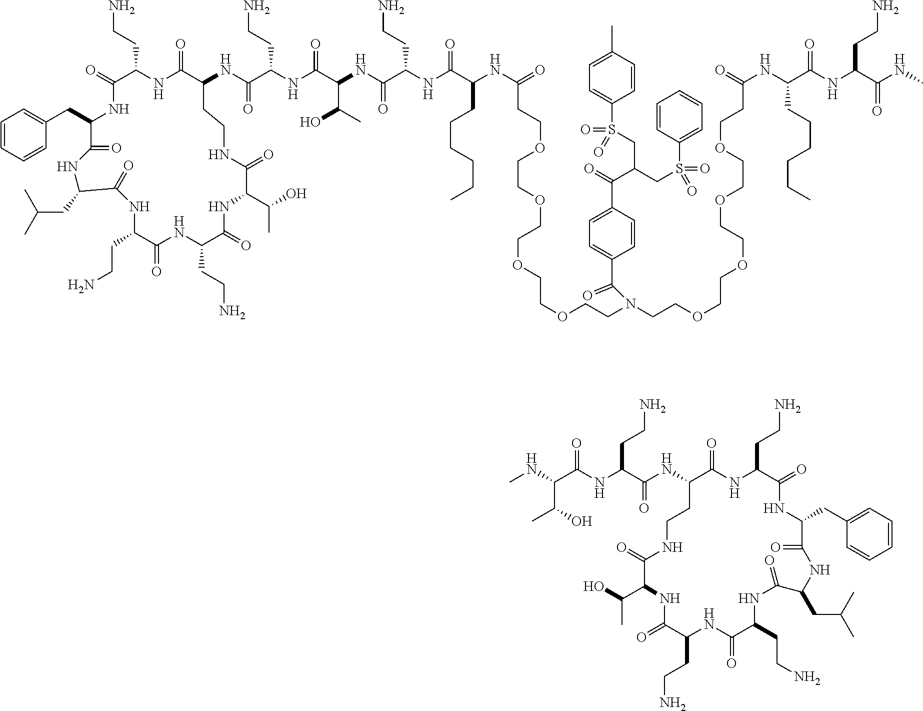

### Step a. Synthesis of Deca Boc Amino PEG 8 Dimer

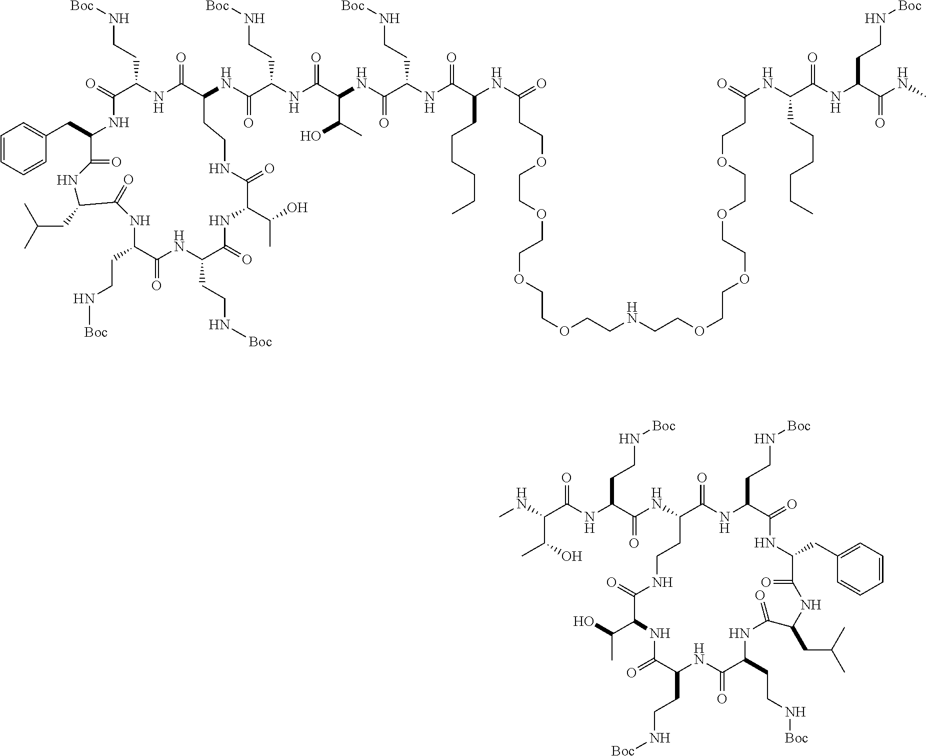

A solution of Int-18 (0.500 g 0.291 mmol), Cbz-amino PEG 8 diacid (0.089 g 0.138 mmol), and DIEA (0.167 mL, 0.960 mmol), in DMF (4 mL), was treated with a solution of HATU (0.122 g, 0.320 mmol), in DMF (1 mL), dropwise over 1 h. After stirring an additional 30 minutes, the reaction was charged with 5% Pd/C (0.5 g), vacuum flushed with hydrogen, and stirred under a hydrogen atmosphere until complete by LCMS. The crude mixture was purified by RPLC using an Isco CombiFlash liquid chromatograph eluted with 30% to 100% methanol and water, using no modifier. Yield 0.212 g, 37% (two steps). Ion(s) found by LCMS: [M−3(Boc)/4]+H+=897.3. [M−4(Boc)/4]+H+=872.3.

### Step b. Synthesis of Bis Sulfone Deca Boc Amino PEG 8 Dimer

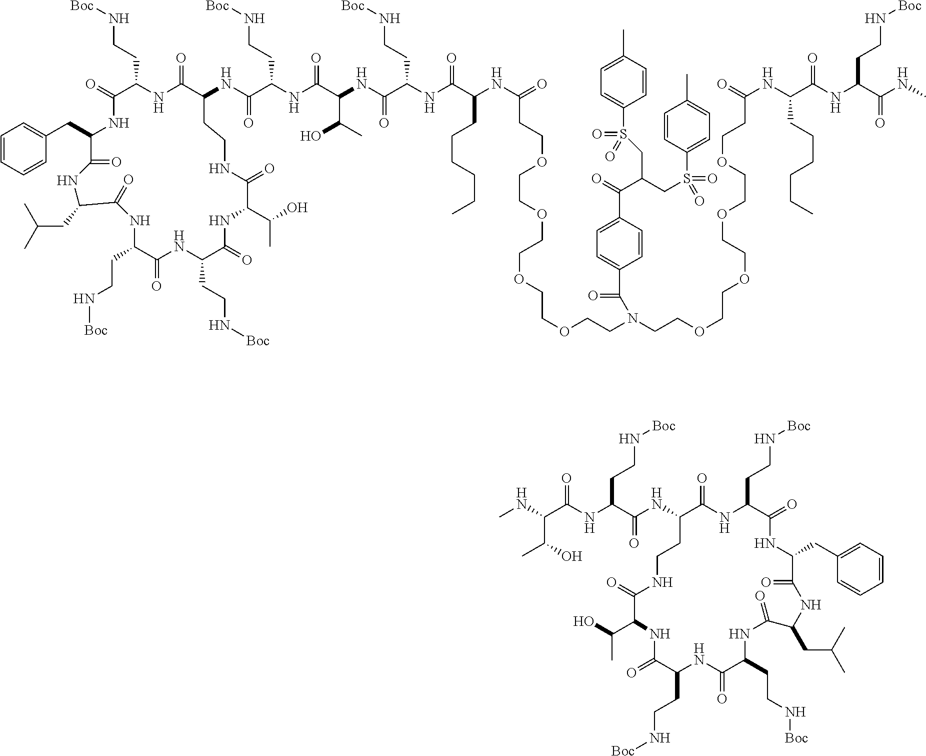

A solution of deca Boc amino PEG 8 dimer (0.212 g, 0.0545 mmol), bis-sulfone acid (0.033 g, 0.0654 mmol), and N-methylmorpholine (0.022 mL, 0.196 mmol), in DMF (3 mL), were treated with a solution of HATU (0.025 g, 0.065 mmol), in DMF (1.0 mL), dropwise over 1 h. The resulting crude reaction mixture was concentrated to an oil and used in the next Step without purification. The product did not ionize by LCMS.

### Step c. Synthesis of Int-11

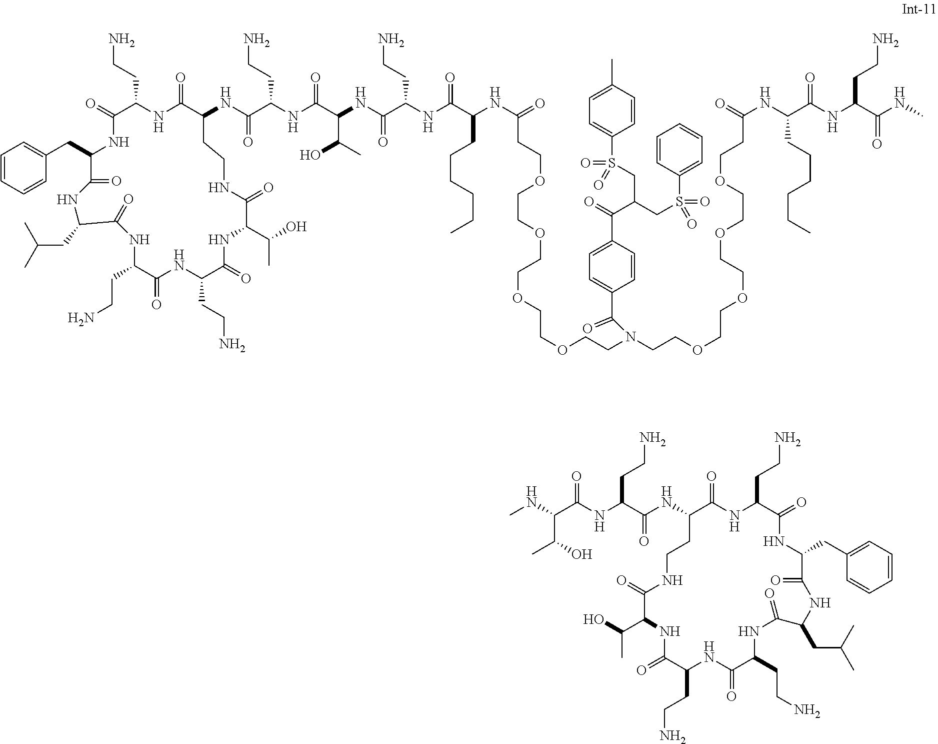

The crude oil was dissolved in DCM (5 mL), and treated with TFA (5 mL), for 5 minutes at room temperature. The resulting solution was concentrated and purified by RPLC using an Isco CombiFlash liquid chromatograph eluted with 10% to 100% methanol and water, using 0.1% TFA as the modifier. Yield 0.099 g, 40% (two steps). Ion(s) found by LCMS: [M/3]+H+=1123.3, [M/4]+H+=842.7.

### Example 14—Conjugate 1 (Conjugate of h-IgG1 Fc-2 (Int-4))

A solution of h-IgG1 Fc (SEQ ID NO: 4) (4.66 mg in 175 μL of pH 7.4 PBS, MW=54,731 Da), Tris pH=8.0 (155 μL), and TCEP (68 μL of 10 mM stock in pH 8.0 Tris) were mixed at room temperature. LCMS after 1 hr showed complete reduction. Int-4 was added as solution (153 μL of 8.04 mg dissolved in 250 μL of pH 8.0 Tris, 8 eq/disulfide). LCMS after 12 h showed that all the reduced Fc was consumed, and a product peak with mass=32,380 Da (DAR=2). The solution was diluted with 15 mL pH 7.4 PBS and concentrated using a centrifugal concentrator (10,000 MWCO) to a volume of 1 mL, two times. The crude mixture was diluted 1:10 in PBS pH 7.4, and purified using MabSelect Sure Resin (GE Healthcare, Chicago, Ill., USA), followed by size exclusion chromatography. Purified material was quantified using a Nanodrop™ UV visible spectrophotometer (using a calculated extinction coefficient based on the amino acid sequence of h-IgG1), and concentrated to approximately 10 mg/mL using a centrifugal concentrator (10,000 MWCO). Yield 0.001 g, 38% after purification. Product peak found by LCMS 32,380 Da (DAR=2/Fc monomer, 4/Fc dimer). Final product: Conjugate 1.

### Example 15—Conjugate 2 (Conjugate of m-IgG2a Fc-3 (Int-4))

A solution of m-IgG2a Fc (SEQ ID NO: 2) (4.5 mg in 315 μL pH 7.4 PBS, MW-54,000 Da), and TCEP (66 μL of 10 mM stock in pH 8.0 Tris) were mixed at room temperature. LCMS after 1 hr showed complete reduction. Int-4 was added as powder (7.9 mg, 8 eq/hinge disulfide). LCMS after 12 h showed that all the reduced Fc was consumed, and a product peak with mass=36.512 Da (DAR=3). The solution was diluted with 15 mL pH 7.4 PBS and ultrafiltered (10,000 MWCO) to a volume of 1 mL, two times. The crude mixture was diluted 1:10 in PBS pH 7.4, and purified using MabSelect Sure Resin (GE Healthcare, Chicago, Ill., USA), followed by size exclusion chromatography. Purified material was quantified using a Nanodrop™ UV visible spectrophotometer (using a calculated extinction coefficient based on the amino acid sequence of m-IgG2a), and concentrated to approximately 10 mg/mL using a centrifugal concentrator (10,000 MWCO). Yield 0.004 g, 69% after purification. Product peak found by LCMS 36,512 Da (DAR=3/Fc monomer, 6/Fc dimer). Final product: Conjugate 2.

### Example 16—Conjugate 3 (Conjugate of m-IgG2a Fc-8 (Int-b))

Peg4-azidoNHS ester (2.2 mg in 2.2 μL of pH 7.4 PBS 1× buffer solution) was added to a solution of m-IgG2a Fc (SEQ ID NO: 2) (28 mg in 300 μL pH 7.4 PBS, MW-54,000 Da) and the mixture was shaken gently for 12 hours at ambient temperature.

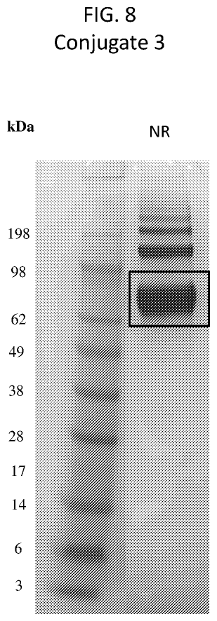

The crude mixture was diluted 1:10 in PBS pH 7.4, and purified using MabSelect Sure Resin (GE Healthcare, Chicago, Ill., USA). Purified material was quantified using a Nanodrop™ UV visible spectrophotometer (using a calculated extinction coefficient based on the amino acid sequence of m-IgG2a), and concentrated to approximately 10 mg/mL using a centrifugal concentrator (10,000 MWCO). Int-5 (28 mg, 7.8 μmol) in a solution of 10 nM sodium ascorbate, 10 mM Copper (II) sulfate and 10 mM tris(3-hydroxylpropyltriazolylmethyl)amine in pH 7.4 PBS 1× buffer (3 mL) was added to the purified azido-n-IgG2a conjugate in pH 7.4 PBS 1× buffer and the mixture was shaken gently at ambient temperature for 24 hours. The crude mixture was diluted 1:10 in PBS pH 7.4, and purified using MabSelect Sure Resin (GE Healthcare, Chicago, Ill., USA), followed by size exclusion chromatography. Purified material was quantified using a Nanodrop™ UV visible spectrophotometer (using a calculated extinction coefficient based on the amino acid sequence of m-IgG2a), and concentrated to approximately 10 mg/mL using a centrifugal concentrator (10,000 MWCO). Purified molecules were analyzed using 4-12% Bis Tris SDS PAGE gels by loading 1-2 μg of each molecule into the gel, and staining using instant Blue staining. Each gel included a molecular weight ladder with the indicated molecular weight standards (FIG. 8). “NR” indicates non-reduced SDS-PAGE gel condition. Yield 0.016 g, 38% after purification. MALDI MS analysis showed a broad range peak (75,683-87079) with an average of mass of 81,375 (DAR=4/Fc monomer, 8/Fc dimer). Final product: Conjugate 3.

### Example 17—Conjugate 4 (Conjugate of m-IgG2a(His)₆ Fc-3 (Int-9))

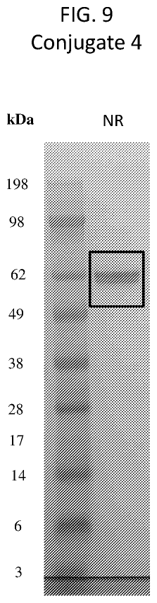

A solution of m-IgG2a(His)₆ Fc (SEQ ID NO: 6) (6.15 mg in 0.5 mL pH 7.4 PBS, MW-59,880 Da), and TCEP (1.85 μL of 500 mM stock in pH 7.4 PBS, 3 eq/disulfide) were mixed at room temperature. LCMS after 1 hr showed complete reduction. The solution was diluted with 15 mL pH 7.8 PBS with 2 mM EDTA and ultrafiltered (10,000 MWCO) to a volume of 1 mL, two times. Int-9 was added as powder (2.11 mg, 1.5 eq/hinge disulfide). LCMS after 1 h at room temperature showed all of the reduced Fc was consumed, and a broad peak with masses corresponding to DAR=2, 3, and 4. The solution was treated with DTT (0.142 mg, 9 eq) and stored at 4 C for 48 h. LCMS showed DAR 3 prior to purification. The crude mixture was diluted 1:10 in PBS pH 7.4, and purified using MabSelect Sure Resin (GE Healthcare, Chicago, Ill., USA), followed by size exclusion chromatography. Purified material was quantified using a Nanodrop™ UV visible spectrophotometer (using a calculated extinction coefficient based on the amino acid sequence of m-IgG2a(His)₆), and concentrated to approximately 10 mg/mL using a centrifugal concentrator (10,000 MWCO). Purified molecules were analyzed using 4-12% Bis Tris SDS PAGE gels by loading 1-2 μg of each molecule into the gel, and staining using instant Blue staining. Each gel included a molecular weight ladder with the indicated molecular weight standards (FIG. 9). “NR” indicates non-reduced SDS-PAGE gel condition. Yield 0.006 g, 83% after purification. Product peak found by LCMS 69,262 Da (DAR=3/Fc dimer). Final product: Conjugate 4.

### Example 18—Conjugate 6 (Conjugate of h-IgG1 Fc-2 (Int-9))

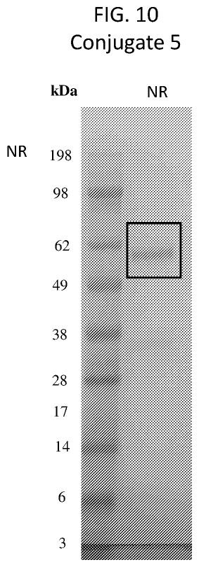

A solution of h-IgG1 Fc (SEQ ID NO: 2) (10.5 mg in 0.6 mL pH 7.4 PBS, MW-54,731 Da), and TCEP (4.6 μL of 500 mM stock in pH 7.4 PBS, 6 eq/disulfide) were mixed at room temperature. LCMS after 1 hr showed complete reduction. The solution was diluted with 15 mL pH 7.8 PBS with 2 mM EDTA and ultrafiltered (10,000 MWCO) to a volume of 1 mL, two times. Int-9 was added as powder (5.2 mg, 3.0 eq/hinge disulfide). LCMS after 1 h at room temperature showed all of the reduced Fc was consumed, and a broad product peak with a mass=61,306 Da(DAR=2). The crude mixture was diluted 1:10 in PBS pH 7.4, and purified using MabSelect Sure Resin (GE Healthcare, Chicago, Ill., USA), followed by size exclusion chromatography. Purified material was quantified using a Nanodrop™ UV visible spectrophotometer (using a calculated extinction coefficient based on the amino acid sequence of h-IgG1), and concentrated to approximately 10 mg/mL using a centrifugal concentrator (10,000 MWCO). Purified molecules were analyzed using 4-12% Bis Tris SDS PAGE gels by loading 1-2 μg of each molecule into the gel, and staining using instant Blue staining. Each gel included a molecular weight ladder with the indicated molecular weight standards (FIG. 10). “NR” indicates non-reduced SDS-PAGE gel condition. Yield 0.010 g, 86% after purification. Product peak found by LCMS 61,306 Da (DAR=2/Fc dimer). Final product: Conjugate 5.

### Example 19—Conjugate 6 (Conjugate of m-IgG2a(His)₆ Fc-2 (Int-5))

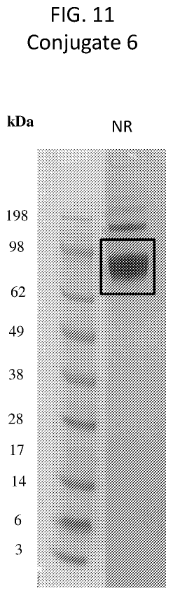

Peg4-azido-NHS ester (4.4 mg in 2.2 mL of pH 7.4 PBS 1× buffer solution) was added to a solution of m-IgG2a(His)₆ Fc (SEQ ID NO: 6) (30 mg in 2.4 mL pH 7.4 PBS, MW-55,600 Da) and the mixture was shaken gently for 12 hours at ambient temperature. The crude mixture was diluted 1:10 in PBS pH 7.4, and purified using MabSelect Sure Resin (GE Healthcare, Chicago, Ill., USA). Purified material was quantified using a Nanodrop™ UV visible spectrophotometer (using a calculated extinction coefficient based on the amino acid sequence of m-IgG2a(His)₆), and concentrated to approximately 10 mg/mL using a centrifugal concentrator (10,000 MWCO). Int-5 (28 mg, 7.8 μmol) in a solution of 10 nM sodium ascorbate, 10 mM Copper (II) sulfate and 10 mM tris(3-hydroxylpropyltriazolylmethyl)amine in pH 7.4 PBS 1× buffer (3 mL) was added to the purified azido-m-IgG2a conjugate in pH 7.4 PBS 1× buffer and the mixture was shaken gently at ambient temperature for 24 hours. The crude mixture was diluted 1:10 in PBS pH 7.4, and purified using MabSelect Sure Resin (GE Healthcare, Chicago, Ill., USA), followed by size exclusion chromatography. Purified material was quantified using a Nanodrop™ UV visible spectrophotometer (using a calculated extinction coefficient based on the amino acid sequence of m-IgG2a(His)₆), and concentrated to approximately 10 mg/mL using a centrifugal concentrator (10,000 MWCO). Purified molecules were analyzed using 4-12% Bis Tris SDS PAGE gels by loading 1-2 μg of each molecule into the gel, and staining using instant Blue staining. Each gel included a molecular weight ladder with the indicated molecular weight standards (FIG. 11). “NR” indicates non-reduced SDS-PAGE gel condition. Yield 0.018 g, 37% after purification. MALDI MS analysis showed a broad range peak (86310-98106) with an average of mass of 89,434 (DAR=6/Fc monomer, 12/Fc dimer). Final product: Conjugate 6.

### Example 20—Conjugate 7 (Conjugate of h-IgG1(His). Fc-2 (Int-10))

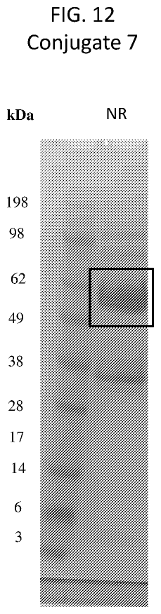

A solution of h-IgG1(His)₆ Fc (SEQ ID NO: 8) (10.0 mg in 1.0 mL pH 7.4 PBS, MW-56,633 Da), and TCEP (6.3 μL of 500 mM stock in pH 7.4 PBS, 9 eq/disulfide) were mixed at room temperature. LCMS after 1 hr showed complete reduction. The solution was diluted with 15 mL pH 7.8 PBS with 2 mM EDTA and ultrafiltered (10,000 MWCO) to a volume of 1 mL, two times. Int-10 was added as powder (5.3 mg, 3.0 eq/hinge disulfide). LCMS after 2 h at room temperature showed all of the reduced Fc was consumed, and a broad product peak, mass=63,682 Da(DAR=2). The reaction was treated with DTT (0.272 mg, 10 eq) and stored at 4 C. The crude mixture was diluted 1:10 in PBS pH 7.4, and purified using MabSelect Sure Resin (GE Healthcare, Chicago, Ill., USA), followed by size exclusion chromatography. Purified material was quantified using a Nanodrop™ UV visible spectrophotometer (using a calculated extinction coefficient based on the amino acid sequence of h-IgG1(His)₆), and concentrated to approximately 10 mg/mL using a centrifugal concentrator (10,000 MWCO). Purified molecules were analyzed using 4-12% Bis Tris SDS PAGE gels by loading 1-2 μg of each molecule into the gel, and staining using instant Blue staining. Each gel included a molecular weight ladder with the indicated molecular weight standards (FIG. 12). “NR” indicates non-reduced SDS-PAGE gel condition. Yield 0.003 g, 24% after purification. Product peak found by LCMS 63,682 Da (DAR=2/Fc dimer). Final product: Conjugate 7.

### Example 21—Conjugate 8 (Conjugate of h-IgG1(His)₆ Fc-2 (Int-7))

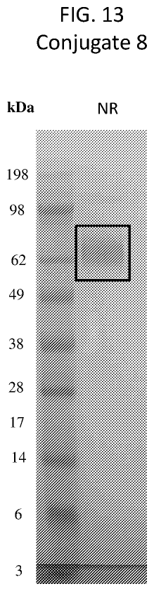

DBCO-peg4-NHS ester (14.4 mg in 2.5 mL of pH 7.4 PBS 1× buffer solution) was added to a solution of h-IgG1(His)6 Fc (SEQ ID NO: 8) (30 mg in 3 mL pH 7.4 PBS, MW-58,633 Da) and the mixture was shaken gently for 12 hours at ambient temperature. Some precipitate was observed during at the end of this time period. The crude mixture was diluted 1:10 in PBS pH 7.4, and purified using MabSelect Sure Resin (GE Healthcare, Chicago, Ill., USA). Purified material was quantified using a Nanodrop™ UV visible spectrophotometer (using a calculated extinction coefficient based on the amino acid sequence of h-IgG1(His)₆), and concentrated to approximately 10 mg/mL using a centrifugal concentrator (10,000 MWCO). Int-7 (28 mg, 7.1 μmol) in a solution of in pH 7.4 PBS 1× buffer (2 mL) was added to the purified azido-h-IgG1 conjugate (4.97 mg) in pH 7.4 PBS 1× buffer (3 mL). Acetonitrile (2 mL) was added when solution began to turn cloudy. The mixture was shaken gently at ambient temperature for 24 hours. The crude mixture was diluted 1:10 in PBS pH 7.4, and purified using MabSelect Sure Resin (GE Healthcare, Chicago, Ill., USA), followed by size exclusion chromatography. Purified material was quantified using a Nanodrop™ UV visible spectrophotometer (using a calculated extinction coefficient based on the amino acid sequence of h-IgG1(His)₆), and concentrated to approximately 10 mg/mL using a centrifugal concentrator (10,000 MWCO). Purified molecules were analyzed using 4-12% Bis Tris SDS PAGE gels by loading 1-2 μg of each molecule into the gel, and staining using instant Blue staining. Each gel included a molecular weight ladder with the indicated molecular weight standards (FIG. 13). “NR” indicates non-reduced SDS-PAGE gel condition. Yield 0.005 g, 14% after purification. MALDI MS analysis showed a broad range peak (60893-79801) with an average of mass of 68.815 (DAR=2/Fc monomer, 4/Fc dimer). Final product: Conjugate 8.

### Example 22—Conjugate 9 (Conjugate of h-IgG1(His)₆ Fc-4 (Int-10))

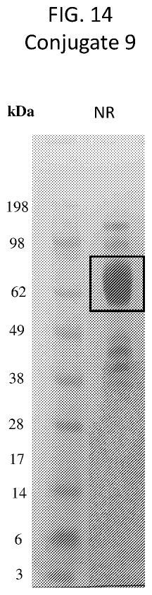

A solution of engineered h-IgG1(His)₆ Fc (SEQ ID NO: 10) (10.0 mg in 0.84 mL pH 7.4 PBS, MW-57,676 Da), and TCEP (12.5 μL of 500 mM stock in pH 7.4 PBS, 9 eq/disulfide) were mixed at room temperature. LCMS after 1 hr showed complete reduction. The solution was diluted with 15 mL pH 7.8 PBS with 2 mM EDTA and ultrafiltered (10,000 MWCO) to a volume of 1 mL, two times. Int-10 was added as powder (6.87 mg, 2.0 eq/hinge disulfide). LCMS after 24 h at room temperature showed all the reduced Fc was consumed, and a broad product peak with masses=64,726 Da(DAR=2), 71,915 Da(DAR=4), and 78,818 Da (DAR=6). The crude mixture was diluted 1:10 in PBS pH 7.4, and purified using MabSelect Sure Resin (GE Healthcare, Chicago, Ill., USA), followed by size exclusion chromatography. Purified material was quantified using a Nanodrop™ UV visible spectrophotometer (using a calculated extinction coefficient based on the amino acid sequence of h-IgG1(His)₆), and concentrated to approximately 10 mg/mL using a centrifugal concentrator (10,000 MWCO). Purified molecules were analyzed using 4-12% Bis Tris SDS PAGE gels by loading 1-2 μg of each molecule into the gel, and staining using instant Blue staining. Each gel included a molecular weight ladder with the indicated molecular weight standards (FIG. 14). “NR” indicates non-reduced SDS-PAGE gel condition. Yield 0.003 g, 24% after purification. Product peak (major) found by LCMS 71915 Da (DAR=4/engineered Fc dimer). Final product: Conjugate 9.

### Example 23—Conjugate 10 (Conjugate of h-IgG1(His). Fc-4 (Int-9))

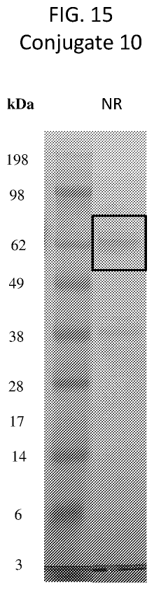

A solution of h-IgG1(His)₆ Fc (SEQ ID NO: 10) (10.0 mg in 0.84 mL pH 7.4 PBS, MW-57,676 Da), and TCEP (12.5 μL of 500 mM stock in pH 7.4 PBS, 9 eq/disulfide) were mixed at room temperature. LCMS after 1 hr showed complete reduction. The solution was diluted with 15 mL pH 7.8 PBS with 2 mM EDTA and ultrafiltered (10,000 MWCO) to a volume of 1 mL, two times. Int-9 was added as powder (6.87 mg, 2.0 eq/hinge disulfide). LCMS after 1 h at room temperature showed all of the reduced Fc was consumed, and a broad product peak with a mass=70,223 Da(DAR=4). The crude mixture was diluted 1:10 in PBS pH 7.4, and purified using MabSelect Sure Resin (GE Healthcare, Chicago, Ill., USA), followed by size exclusion chromatography. Purified material was quantified using a Nanodrop™ UV visible spectrophotometer (using a calculated extinction coefficient based on the amino acid sequence of h-IgG1(His)₆), and concentrated to approximately 10 mg/mL using a centrifugal concentrator (10,000 MWCO). Purified molecules were analyzed using 4-12% Bis Tris SDS PAGE gels by loading 1-2 μg of each molecule into the gel, and staining using instant Blue staining. Each gel included a molecular weight ladder with the indicated molecular weight standards (FIG. 15). “NR” indicates non-reduced SDS-PAGE gel condition. Yield 0.010 g, 82% after purification. Product peak (major) found by LCMS 70,223 Da (DAR=4/engineered Fc dimer). Final product: Conjugate 10.

### Example 24—Conjugate 11 (Conjugate of h-IgG1(His)₆ Fc-4 (Int-11))

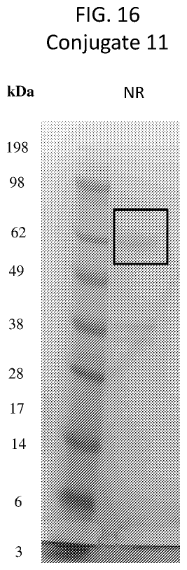

A solution of h-IgG1(His). Fc (SEQ ID NO: 10) (10.0 mg in 0.84 mL pH 7.4 PBS, MW-57,676 Da), and TCEP (12.5 μL of 500 mM stock in pH 7.4 PBS, 9 eq/disulfide) were mixed at room temperature. LCMS after 1 hr showed complete reduction. The solution was diluted with 15 mL pH 7.8 PBS with 2 mM EDTA and ultrafiltered (10,000 MWCO) to a volume of 1 mL, two times. Int-11 was added as powder (6.31 mg, 2.0 eq/hinge disulfide). LCMS after 1 h at room temperature showed all of the reduced Fc was consumed, and a broad product peak with a mass=69,913 Da(DAR=4). The crude mixture was diluted 1:10 in PBS pH 7.4, and purified using MabSelect Sure Resin (GE Healthcare, Chicago, Ill., USA), followed by size exclusion chromatography. Purified material was quantified using a Nanodrop™ UV visible spectrophotometer (using a calculated extinction coefficient based on the amino acid sequence of h-IgG1(His)₆), and concentrated to approximately 10 mg/mL using a centrifugal concentrator (10,000 MWCO). Purified molecules were analyzed using 4-12% Bis Tris SDS PAGE gels by loading 1-2 μg of each molecule into the gel, and staining using instant Blue staining. Each gel included a molecular weight ladder with the indicated molecular weight standards (FIG. 16). “NR” indicates non-reduced SDS-PAGE gel condition. Yield 0.010 g, 82% after purification. Product peak (major) found by LCMS 69,913 Da (DAR=4/engineered Fc dimer). Final product: Conjugate 11.

### Example 25—Synthesis of Int-12 (maleimide-PEG 8-amino-polymyxin B)

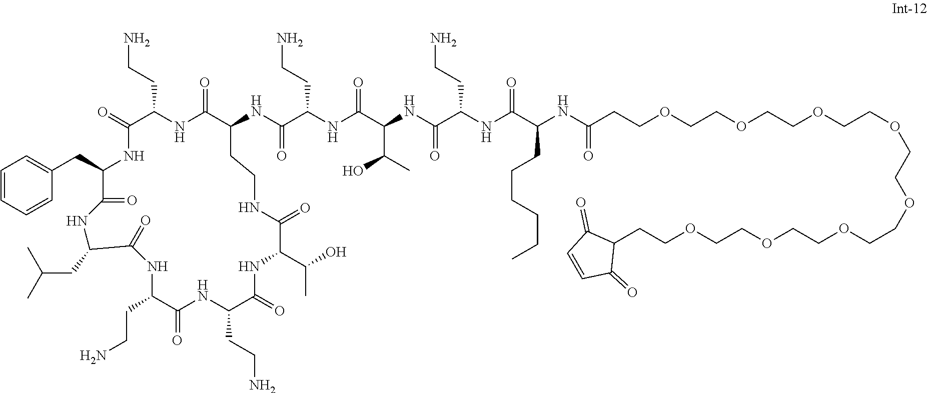 [SMILES: CCCCCC[C@H](NC(=O)CCOCCOCCOCCOCCOCCOCCOCCOCCC1C(=O)C=CC1=O)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O]

### Step a. Synthesis of Penta Boc Polymyxin B Decapeptide (Int-7a)

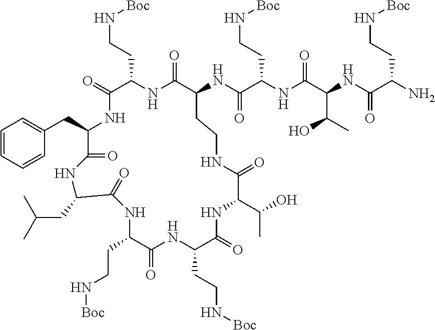 [SMILES: CC(C)C[C@@H]1NC(=O)[C@@H](Cc2ccccc2)NC(=O)[C@H](CCNC(=O)OC(C)(C)C)NC(=O)[C@@H](NC(=O)[C@H](CCNC(=O)OC(C)(C)C)NC(=O)[C@@H](NC(=O)[C@@H](N)CCNC(=O)OC(C)(C)C)[C@@H](C)O)CCNC(=O)[C@H]([C@@H](C)O)NC(=O)[C@H](CCNC(=O)OC(C)(C)C)NC(=O)[C@H](CCNC(=O)OC(C)(C)C)NC1=O]

Tri-Boc polymyxin B heptapeptide (0.464 g, 0.437 mmol, Int-1), and tripeptide acid (0.316 g, 0.398 mmol, Int-3), were dissolved in DMF (1 mL), DIEA (0.229 mL, 1.31 mmol), then treated with HATU (0.166 g, 0.437 mmol). After stirring for 30 min LCMS showed complete conversion. The crude Cbz-product was diluted with methanol (4 mL), and charged with 5% Pd/C (0.250 g) and placed under a hydrogen atmosphere. After 2 hr the mixture was filtered through Celite, concentrated, and purified by RPLC using an Isco CombiFlash liquid chromatograph eluted with 10% to 100% methanol and water, using no modifier. Yield of Int-7a was 0.640 g, 94% (two steps). Ion(s) found by LCMS: [(M−1Boc)/2]+H+=1054.6. [(M−2Boc)/2]+H+=1004.6.

### Step b. Synthesis of Aminooctanoyl Penta Boc Polymyxin B Decapeptide (Int-18)

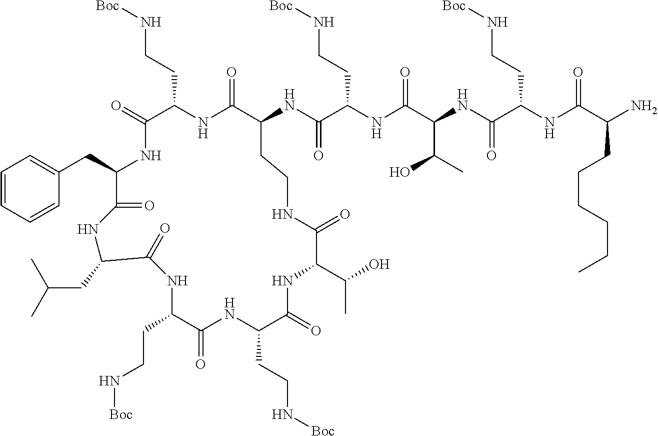 [SMILES: CCCCCC[C@H](N)C(=O)N[C@@H](CCNC(=O)OC(C)(C)C)C(=O)N[C@H](C(=O)N[C@@H](CCNC(=O)OC(C)(C)C)C(=O)N[C@H]1CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](Cc2ccccc2)NC(=O)[C@H](CCNC(=O)OC(C)(C)C)NC1=O)[C@@H](C)O]

A solution of Int-7a (3.00 g, 1.918 mmol), Cbz-aminooctanoic acid (0.591 g, 2.01 mmol), and DIEA (1.05 mL, 6.04 mmol), in DMF (10 mL), was treated with a solution of HATU (0.766 g, 2.01 mmol) dissolved in DMF (3 mL), dropwise over 60 minutes. After 2 h, LCMS showed complete consumption of starting material. The crude mixture was treated with 5% Pd/C (1.7 g), vacuum flushed with hydrogen, and stirred under a hydrogen atmosphere for 2 hr or until complete by LCMS. The crude reaction was filtered through Celite, concentrated, and purified by RPLC using an Isco CombiFlash liquid chromatograph eluted with 20% to 100% methanol and water, using no modifier. Yield of Int-18 was 2.59 g, 79% (two steps). Ion(s) found by LCMS: [M/2]+H+=853.4.

### Step c. Synthesis of Maleimide Penta Boc Polymyxin B Undecapeptide

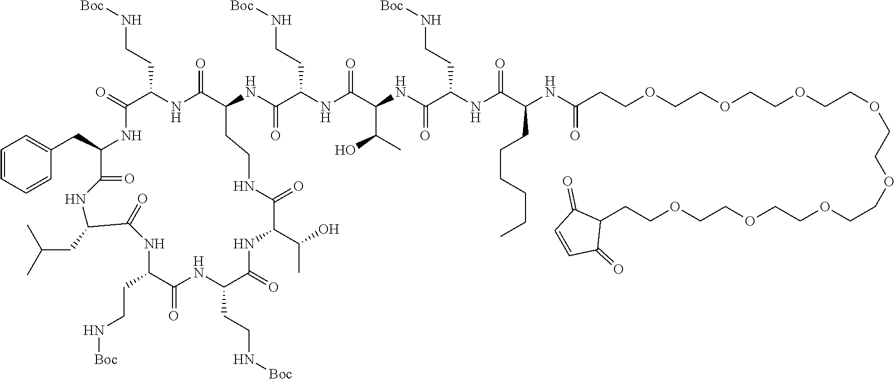 [SMILES: CCCCCC[C@H](NC(=O)CCOCCOCCOCCOCCOCCOCCOCCOCCC1C(=O)C=CC1=O)C(=O)N[C@@H](CCNC(=O)OC(C)(C)C)C(=O)N[C@H](C(=O)N[C@@H](CCNC(=O)OC(C)(C)C)C(=O)N[C@H]1CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](Cc2ccccc2)NC(=O)[C@H](CCNC(=O)OC(C)(C)C)NC1=O)[C@@H](C)O]

A solution of aminooctanoyl penta Boc polymyxin B undecapeptide (0.5 g, 0.293 mmol), maleimide-acid (0.168 g, 0.323 mmol), DIEA (0.153 mL, 0.880 mmol), in DMF (3 mL), was treated with a solution of HATU (0.123 mg, 0.323 mmol) in DMF (1 mL) dropwise over 60 minutes. The resulting mixture was purified by RPLC using an Isco CombiFlash liquid chromatograph eluted with 20% to 100% methanol and water, using no modifier. Yield 0.595 g, 92%. Ion(s) found by LCMS: [M−1(Boc)/2]+H+=1054.6, [M−2(Boc)/2]+H+=1004.6

### Step d. Synthesis of Int-12

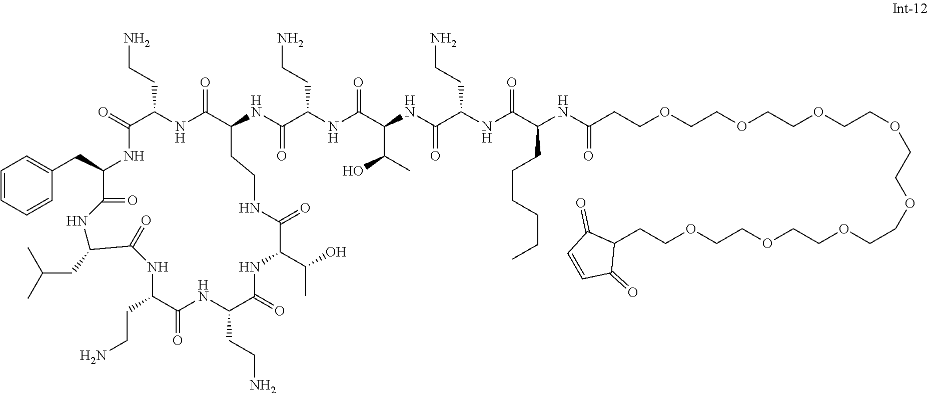 [SMILES: CCCCCC[C@H](NC(=O)CCOCCOCCOCCOCCOCCOCCOCCOCCC1C(=O)C=CC1=O)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O]

A solution of maleimide penta Boc polymyxin B undecapeptide (0.595 g, 0.269 mmol), dissolved in DCM (2 mL), was treated with TFA (2 mL) while stirring for 5 minutes at room temperature. The resulting solution was concentrated and purified by RPLC using an Isco CombiFlash liquid chromatograph eluted with 5% to 100% methanol and water, using TFA (0.1%) as the modifier. Yield 0.560 g, 73%. Ion(s) found by LCMS: [M/3]+H+=570.0, [M/4]+H+=427.7.

### Example 26—Synthesis of Int-13 (Bis Sulfone Peg 8 Polymyxin B)

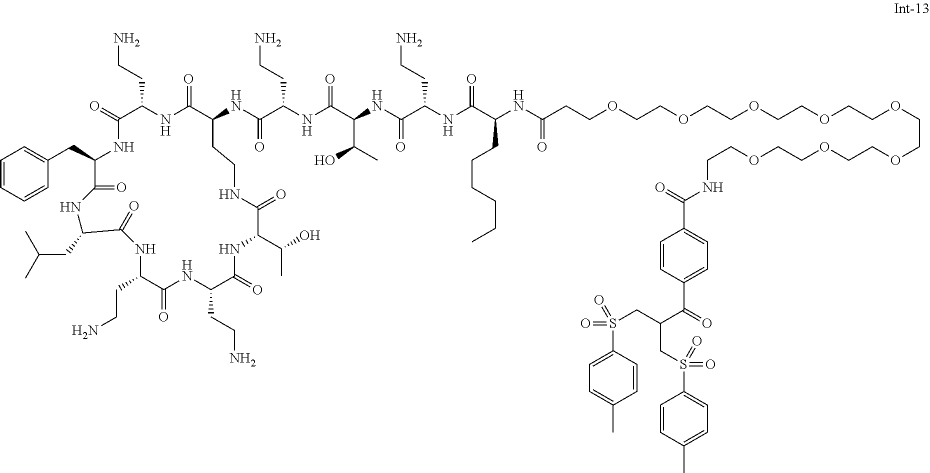 [SMILES: CCCCCC[C@H](NC(=O)CCOCCOCCOCCOCCOCCOCCOCCOCCNC(=O)c1ccc(C(=O)C(CS(=O)(=O)c2ccc(C)cc2)CS(=O)(=O)c2ccc(C)cc2)cc1)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O]

### Step a. Synthesis of Amino PEG 8 Penta Boc Polymyxin B Undecapeptide

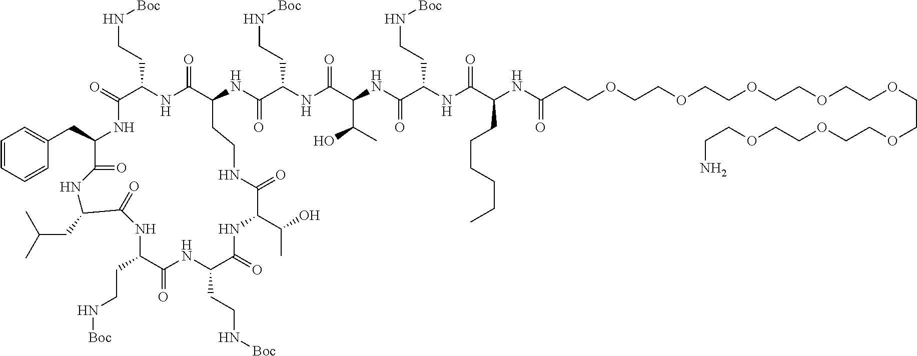 [SMILES: CCCCCC[C@H](NC(=O)CCOCCOCCOCCOCCOCCOCCOCCOCCN)C(=O)N[C@@H](CCNC(=O)OC(C)(C)C)C(=O)N[C@H](C(=O)N[C@@H](CCNC(=O)OC(C)(C)C)C(=O)N[C@H]1CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](Cc2ccccc2)NC(=O)[C@H](CCNC(=O)OC(C)(C)C)NC1=O)[C@@H](C)O]

A solution of aminooctanoyl penta Boc polymyxin B decapeptide (0.600 g, 0.352 mmol. Int-18), Cbz-amino Peg 8 acid (0.223 g, 0.387 mmol), and DIEA (0.202 mL, 1.161 mmol), in DMF (3 mL) was treated with a solution of HATU (0.147 g, 0.387 mmol), in DMF (1 mL), dropwise over 1 hr. After stirring for an additional 30 minutes, the reaction was charged with 5% Pd/C (0.400 g), vacuum flushed with hydrogen, and stirred under a hydrogen atmosphere until complete by LCMS (2 h). The resulting mixture was filtered through celite, concentrated, and purified by RPLC using an Isco CombiFlash liquid chromatograph eluted with 30% to 100% methanol and water, using no modifier. Yield 0.450 g, 64%. Ion(s) found by LCMS: [M/2]+H+=1064.6. [M−1(Boc)/2]+H+=1014.6.

### Step b. Synthesis of PEG 8 Penta Boc Polymyxin B Undecapeptide

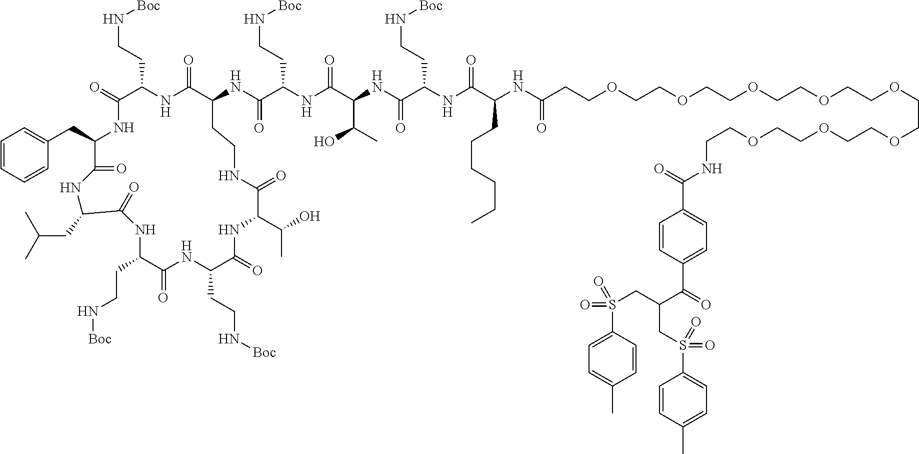 [SMILES: CCCCCC[C@H](NC(=O)CCOCCOCCOCCOCCOCCOCCOCCOCCNC(=O)c1ccc(C(=O)C(CS(=O)(=O)c2ccc(C)cc2)CS(=O)(=O)c2ccc(C)cc2)cc1)C(=O)N[C@@H](CCNC(=O)OC(C)(C)C)C(=O)N[C@H](C(=O)N[C@@H](CCNC(=O)OC(C)(C)C)C(=O)N[C@H]1CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](Cc2ccccc2)NC(=O)[C@H](CCNC(=O)OC(C)(C)C)NC1=O)[C@@H](C)O]

A solution of amino Peg 8 penta Boc polymyxin B undecapeptide (0.450 g, 0.211 mmol), bissulfone acid (0.116 g, 0.233 mmol), and N-methylmorpholine (0.051 mL, 0.465 mmol) in DMF (4 mL), was treated with a solution of HATU (0.0884 g, 0.232 mmol), in DMF (1.0 mL), dropwise over 1 hr. After stirring for an additional 30 minutes the reaction was purified by RPLC using an Isco CombiFlash liquid chromatograph eluted with 30% to 100% methanol and water, using no modifier. Yield 0.475 g, 86%. Ion(s) found by LCMS: No ions observed.

### Step c. Synthesis of Bis Sulfone PEG 8 Polymyxin B (Int-13)

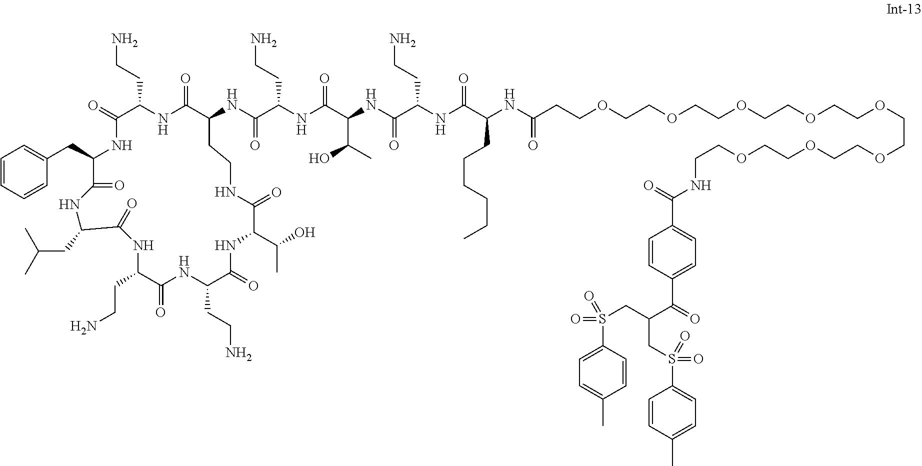 [SMILES: CCCCCC[C@H](NC(=O)CCOCCOCCOCCOCCOCCOCCOCCOCCNC(=O)c1ccc(C(=O)C(CS(=O)(=O)c2ccc(C)cc2)CS(=O)(=O)c2ccc(C)cc2)cc1)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O]

A solution of Peg 8 penta Boc polymyxin B undecapeptide (0.475 mg, 0.182 mmol), suspended in DCM (3 mL) was treated with TFA (3 mL) for 5 minutes at room temperature, then concentrated to an oil. The crude oil was purified by RPLC using an Isco CombiFlash liquid chromatograph eluted with 5% to 100% methanol and water, using TFA (0.1%) as the modifier. Yield 0.475 g, 97%. Ion(s) found by LCMS: [M/3]+H+=704.0, [M14]+H+=528.3.

### Example 27—Synthesis of Int-14 (Bis Sulfone Peg 4 Polymyxin B)

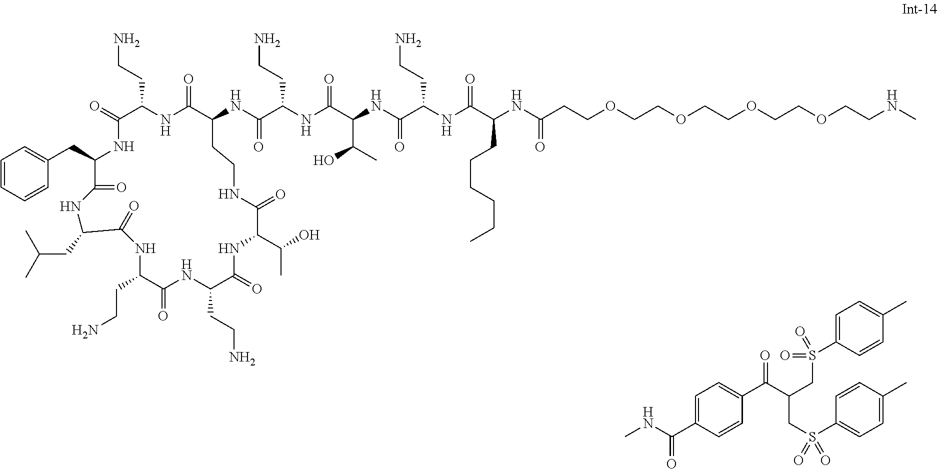

The title compound was prepared analogously to Int-13, where Cbz-amino PEG 4 acid was used in place of Cbz-amino Peg 8 acid in the first Step of that sequence. Ion(s) found by LCMS: [M/2]+H+=967.5, [M/3]+H+=645.3.

### Example 28—Conjugate 12 (Conjugate of m-IgG2a Fc-3 (Int-12))

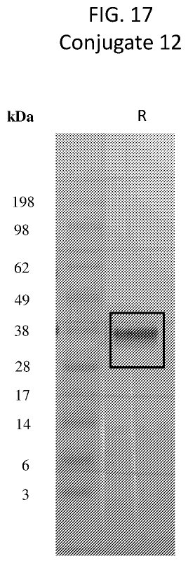

A solution of m-IgG2a Fc (SEQ ID NO: 2) (10 mg in 169 μL pH 7.4 PBS, MW-54,000 Da), and TCEP (2.8 μL of 50 mM stock in pH 8.0 Tris) were mixed at room temperature. LCMS after 1 hr showed complete reduction. Int-12 was added as powder (19.0 mg, 6 eq/hinge disulfide). LCMS after 48 h at 4 C showed that all the reduced Fc was consumed. The solution was diluted with 15 mL pH 7.4 PBS and ultrafiltered (10,000 MWCO) to a volume of 1 mL, two times. The crude mixture was diluted 1:10 in PBS pH 7.4, and purified using MabSelect Sure Resin (GE Healthcare, Chicago, Ill., USA), followed by size exclusion chromatography. Purified material was quantified using a Nanodrop™ UV visible spectrophotometer (using a calculated extinction coefficient based on the amino acid sequence of m-IgG2a), and concentrated to approximately 10 mg/mL using a centrifugal concentrator (10,000 MWCO). Purified molecules were analyzed using 4-12% Bis Tris SDS PAGE gels by loading 1-2 μg of each molecule into the gel, and staining using instant Blue staining. Each gel included a molecular weight ladder with the indicated molecular weight standards (FIG. 17). “R” indicates reduced SDS-PAGE gel condition. Yield 0.008 g, 70% after purification. Product peak found by LCMS 31,272 Da (DAR=3/Fc monomer, 6/Fc dimer). Final product: Conjugate 12.

### Example 29—Conjugate 13 (Conjugate of h-IgG1 Fc-2 (Int-13))

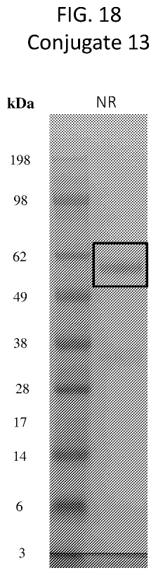

A solution of h-IgG1 Fc (SEQ ID NO: 4) (25 mg in 1 mL pH 7.4 PBS, MW=54,731 Da), and TCEP (9.1 μL of 50 mM stock in pH 7.4 PBS, 5 eq/disulfide) were mixed at room temperature. LCMS after 1 hr showed complete reduction. The solution was diluted with 15 mL pH 7.8 PBS w/2 mM EDTA and ultrafiltered (10,000 MWCO) to a volume of 1 mL, two times. Int-13 was added as powder (14.7 mg, 6 eq/hinge disulfide). LCMS after 12 h at room temperature showed all the reduced Fc was consumed, and a product peak with mass=58,329 Da(DAR=2). The solution was diluted with 15 mL pH 7.4 PBS and ultrafiltered (10,000 MWCO) to a volume of 1 mL, two times. The crude mixture was diluted 1:10 in PBS pH 7.4, and purified using MabSelect Sure Resin (GE Healthcare, Chicago, Ill., USA), followed by size exclusion chromatography. Purified material was quantified using a Nanodrop™ UV visible spectrophotometer (using a calculated extinction coefficient based on the amino acid sequence of h-IgG1), and concentrated to approximately 10 mg/mL using a centrifugal concentrator (10,000 MWCO). Purified molecules were analyzed using 4-12% Bis Tris SDS PAGE gels by loading 1-2 μg of each molecule into the gel, and staining using instant Blue staining. Each gel included a molecular weight ladder with the indicated molecular weight standards (FIG. 18). Yield 0.022 g, 83% after purification. Product peak found by LCMS 58,329 Da (DAR=2/Fc dimer). Final product: Conjugate 13.

### Example 30—Conjugate 14 (Conjugate of h-IgG1 Fc-2 (Int-14))

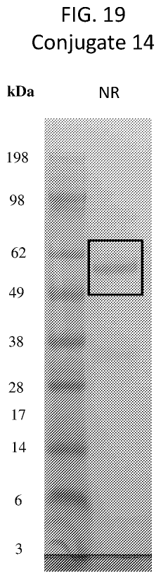

A solution of h-IgG1 Fc (SEQ ID NO: 4) (25 mg in 1 mL pH 7.4 PBS, MW=54,731 Da), and TCEP (3.6 μL of 500 mM stock in pH 7.4 PBS, 2 eq/disulfide) were mixed at room temperature. LCMS after 1 hr showed complete reduction. The solution was diluted with 15 mL pH 7.8 PBS w/2 mM EDTA and ultrafiltered (10,000 MWCO) to a volume of 1 mL, two times. Int-14 was added as powder (13.7 mg, 6 eq/hinge disulfide). LCMS after 12 h at room temperature showed all the reduced Fc was consumed, and a broad product peak with mass=57,977 Da(DAR=2). The solution was diluted with 15 mL pH 7.4 PBS and ultrafiltered (10,000 MWCO) to a volume of 1 mL, two times. The crude mixture was diluted 1:10 in PBS pH 7.4, and purified using MabSelect Sure Resin (GE Healthcare, Chicago, Ill., USA), followed by size exclusion chromatography. Purified material was quantified using a Nanodrop™ UV visible spectrophotometer (using a calculated extinction coefficient based on the amino acid sequence of h-IgG1), and concentrated to approximately 10 mg/mL using a centrifugal concentrator (10,000 MWCO). Purified molecules were analyzed using 4-12% Bis Tris SDS PAGE gels by loading 1-2 μg of each molecule into the gel, and staining using instant Blue staining. Each gel included a molecular weight ladder with the indicated molecular weight standards (FIG. 19). Yield 0.021 g, 78% after purification. Product peak found by LCMS 57,977 Da (DAR=2/Fc dimer). Final product: Conjugate 14.

### Example 31—Conjugate 15 (Conjugate of m-IgG2a(His)₆ Fc-3 (Int-13))

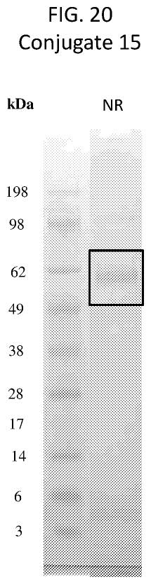

A solution of m-IgG2a(His)₆ Fc (SEQ ID NO: 6) (12.8 mg in 1 mL pH 7.4 PBS, MW-59,880 Da), and TCEP (3.8 μL of 500 mM stock in pH 7.4 PBS, 3 eq/disulfide) were mixed at room temperature. LCMS after 1 hr showed complete reduction. The solution was diluted with 15 mL pH 7.8 PBS w/2 mM EDTA and ultrafiltered (10,000 MWCO) to a volume of 1 mL, two times. Int-13 was added as powder (3.4 mg, 2 eq/hinge disulfide). LCMS after 1 h at room temperature showed all of the reduced Fc was consumed, and a product peak with mass=65,438 Da(DAR=3). The solution was diluted with 15 mL pH 7.4 PBS and ultrafiltered (10,000 MWCO) to a volume of 1 mL, two times. The crude mixture was diluted 1:10 in PBS pH 7.4, and purified using MabSelect Sure Resin (GE Healthcare, Chicago, Ill., USA), followed by size exclusion chromatography. Purified material was quantified using a Nanodrop™ UV visible spectrophotometer (using a calculated extinction coefficient based on the amino acid sequence of m-IgG2a(His)G), and concentrated to approximately 10 mg/mL using a centrifugal concentrator (10,000 MWCO). Purified molecules were analyzed using 4-12% Bis Tris SDS PAGE gels by loading 1-2 μg of each molecule into the gel, and staining using instant Blue staining. Each gel included a molecular weight ladder with the indicated molecular weight standards (FIG. 20). Yield 0.013 g, 94% after purification. Product peak found by LCMS 65,438 Da (DAR=3/Fc dimer). Final product: Conjugate 15.

### Example 32—Conjugate 16 (Conjugate of h-IgG1(His). Fc-2 (Int-5))

Peg4-azido-NHS ester (38.4 mg in 150 uL of DMF) was added to a solution of h-IgG1(His)₆ Fc (SEQ ID NO: 8 (140 mg in 15 mL pH 7.4 PBS 1× buffer, MW-55,111 Da) and the mixture was shaken gently for 12 hours at ambient temperature.

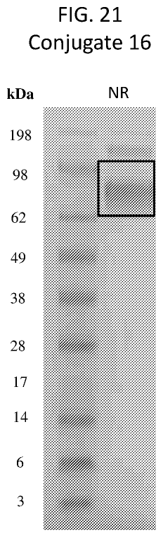

The crude mixture was diluted 1:10 in PBS pH 7.4, and purified using MabSelect Sure Resin (GE Healthcare, Chicago, Ill., USA). Purified material was quantified using a Nanodrop™ UV visible spectrophotometer (using a calculated extinction coefficient based on the amino acid sequence of h-IgG1(His)₆), and concentrated to approximately 10 mg/mL using a centrifugal concentrator (10,000 MWCO). Int-5 (150 mg, 41.9 μmol) was added to the purified azido-h-IgG1 conjugate (56.9 mg in 7 mL of pH 7.4 PBS 1× buffer, MW-58703 Da by MALDI) followed by 2 mL of a solution of 10 mM sodium ascorbate, 10 mM Copper (II) sulfate and 10 mM tris(3-hydroxylpropyltriazolylmethyl)amine in pH 7.4 PBS 1× buffer and the mixture was shaken gently at ambient temperature for 4 hours at which point an additional 0.6 mL of the solution of 10 mM sodium ascorbate, 10 mM Copper (II) sulfate and 10 mM tris(3-hydroxylpropyltriazolylmethyl)amine in pH 7.4 PBS 1× buffer was added and the mixture was shaken gently for 12 hours. The crude mixture was diluted 1:10 in PBS pH 7.4, and purified using MabSelect Sure Resin (GE Healthcare, Chicago, Ill., USA), followed by size exclusion chromatography. Purified material was quantified using a Nanodrop™ UV visible spectrophotometer (using a calculated extinction coefficient based on the amino acid sequence of h-IgG1(His)₆), and concentrated to approximately 10 mg/mL using a centrifugal concentrator (10,000 MWCO). Purified molecules were analyzed using 4-12% Bis Tris SDS PAGE gels by loading 1-2 μg of each molecule into the gel, and staining using instant Blue staining. Each gel included a molecular weight ladder with the indicated molecular weight standards (FIG. 21). “NR” indicates non-reduced SDS-PAGE gel condition. Yield 0.057 g, 78% after purification. MALDI MS analysis showed a broad range peak (60893-79801) with an average of mass of 80443 (DAR=4/Fc monomer, 8/Fc dimer). Final product: Conjugate 16.

### Example 33—Conjugate 17 (Conjugate of Aglycosylated h-IgG1(his)₆ Fc-2 (Int-4))

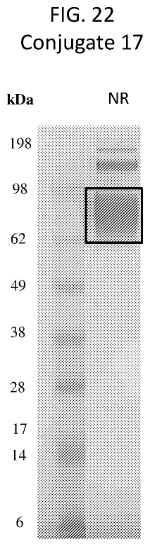

Peg4-azido-NHS ester (20.4 mg in 82 uL of DMF) was added to a solution of aglycosylated h-IgG1(His)₆ Fc (SEQ ID NO: 14) (74 mg in 8 mL pH 7.4 PBS 1× buffer, MW-53,637 Da) and the mixture was shaken gently for 12 hours at ambient temperature. The crude mixture was diluted 1:10 in PBS pH 7.4, and purified using MabSelect Sure Resin (GE Healthcare, Chicago, Ill., USA). Purified material was quantified using a Nanodrop™ UV visible spectrophotometer (using a calculated extinction coefficient based on the amino acid sequence of h-IgG1(His)₆), and concentrated to approximately 10 mg/mL using a centrifugal concentrator (10,000 MWCO). Int-5 (150 mg, 41.9 μmol) was added to the purified azido-h-IgG2a conjugate(azido-CTP-125) (56.9 mg in 7.5 mL of pH 7.4 PBS 1× buffer, MW˜57,118 Da by MALDI) followed by 2 mL of a solution of 10 mM sodium ascorbate, 10 mM Copper (II) sulfate and 10 mM tris(3-hydroxylpropyltriazolylmethyl)amine in pH 7.4 PBS 1× buffer and the mixture was shaken gently at ambient temperature for 4 hours at which point an additional 0.6 mL of the solution of 10 mM sodium ascorbate, 10 mM Copper (II) sulfate and 10 mM tris(3-hydroxylpropyltriazolylmethyl)amine in pH 7.4 PBS 1× buffer was added and the mixture was shaken gently for an additional 12 hours. The crude mixture was diluted 1:10 in PBS pH 7.4, and purified using MabSelect Sure Resin (GE Healthcare, Chicago, Ill., USA), followed by size exclusion chromatography. Purified material was quantified using a Nanodrop™ UV visible spectrophotometer (using a calculated extinction coefficient based on the amino acid sequence of h-IgG1(His)₆), and concentrated to approximately 10 mg/mL using a centrifugal concentrator (10,000 MWCO). Purified molecules were analyzed using 4-12% Bis Tris SDS PAGE gels by loading 1-2 μg of each molecule into the gel, and staining using instant Blue staining. Each gel included a molecular weight ladder with the indicated molecular weight standards (FIG. 22). “NR” indicates non-reduced SDS-PAGE gel condition. Yield 0.055 g, 50% after purification. MALDI MS analysis showed a broad range peak (60893-79801) with an average of mass of 80899 (DAR=4.5/Fc monomer, 9/Fc dimer). Final product: Conjugate 17.

### Example 34—Synthesis of Int-15

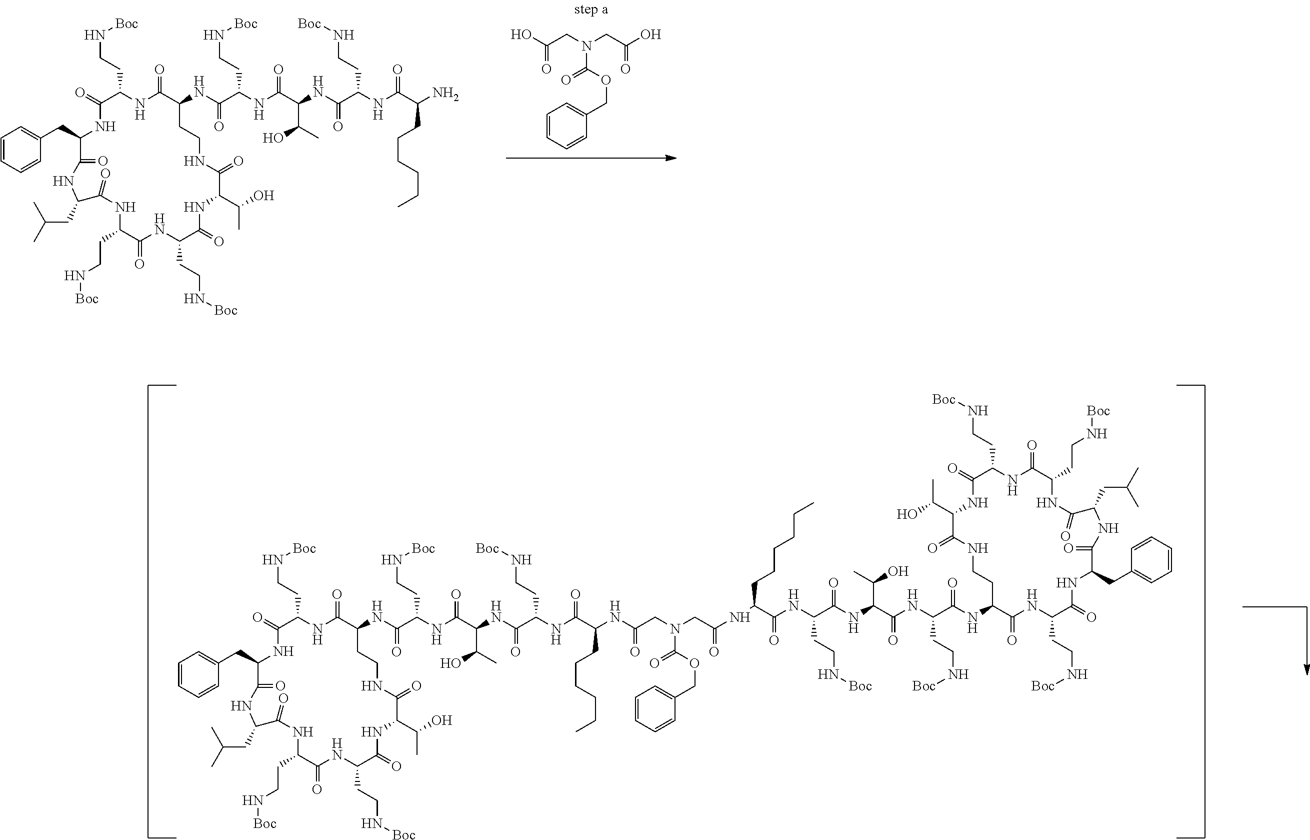

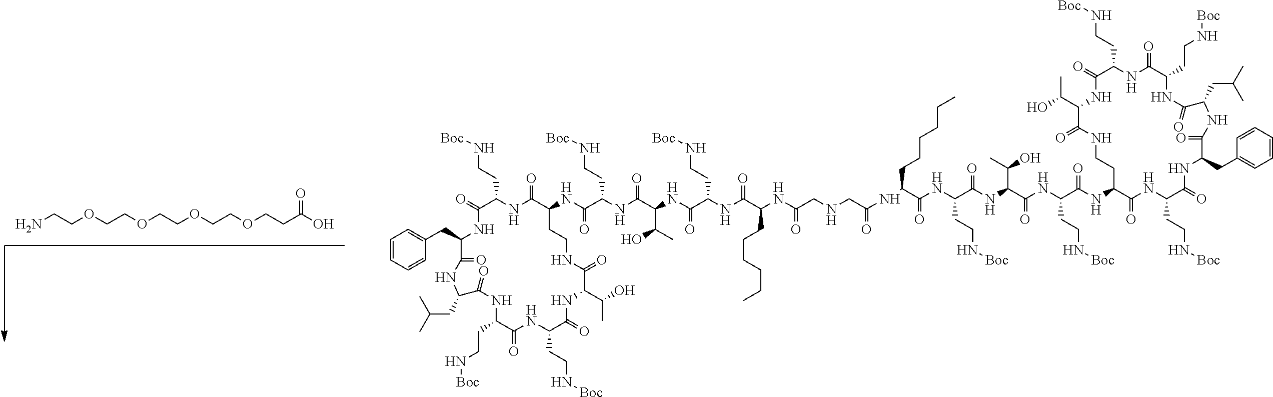

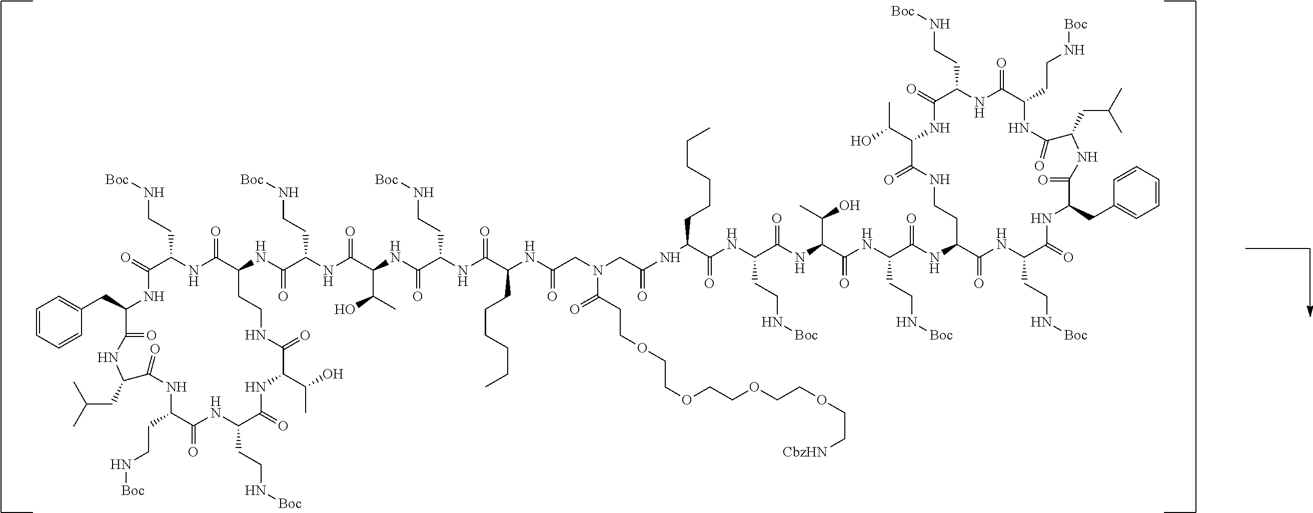

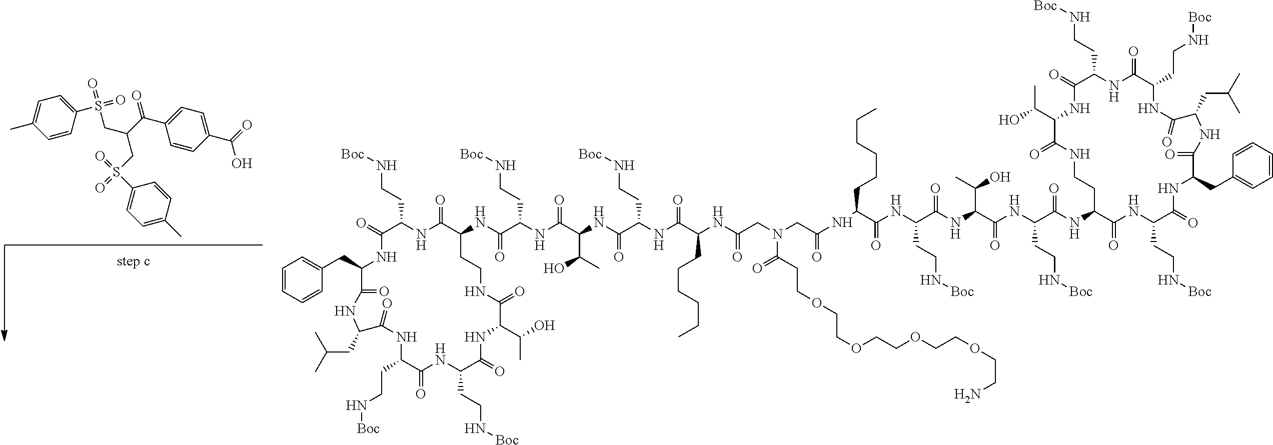

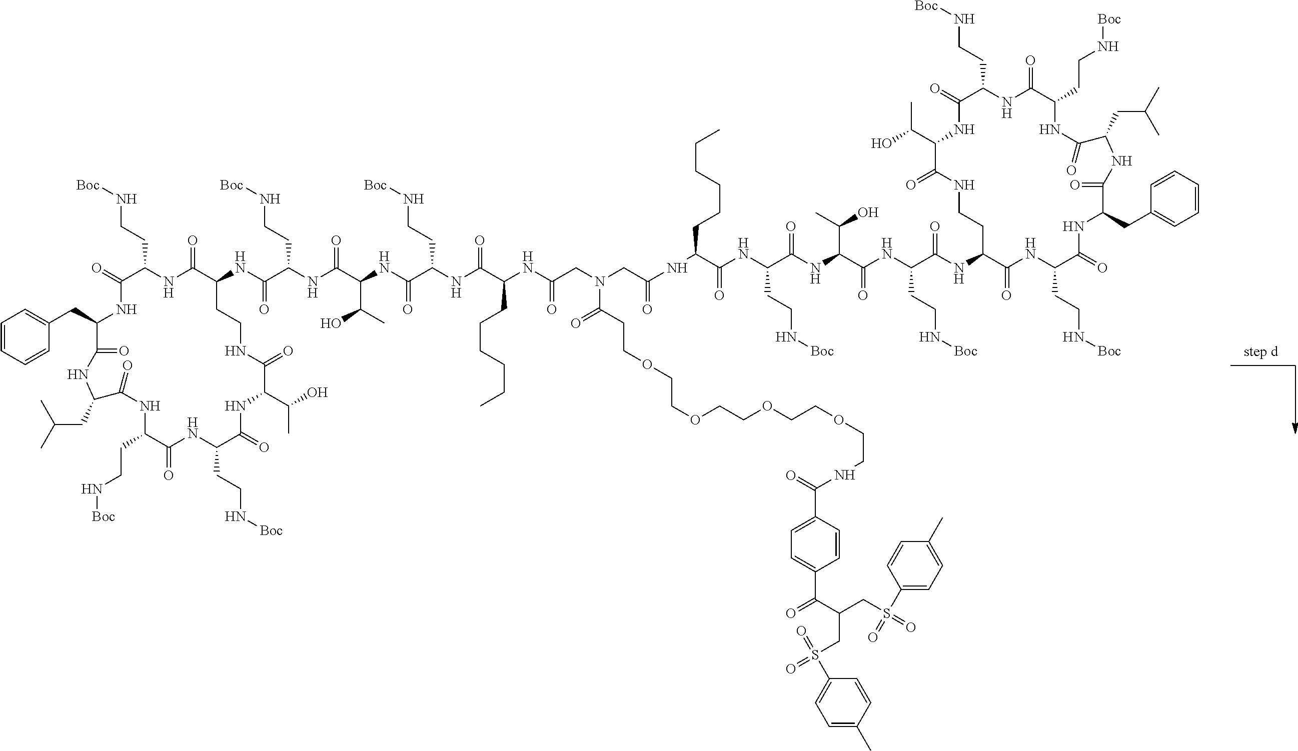

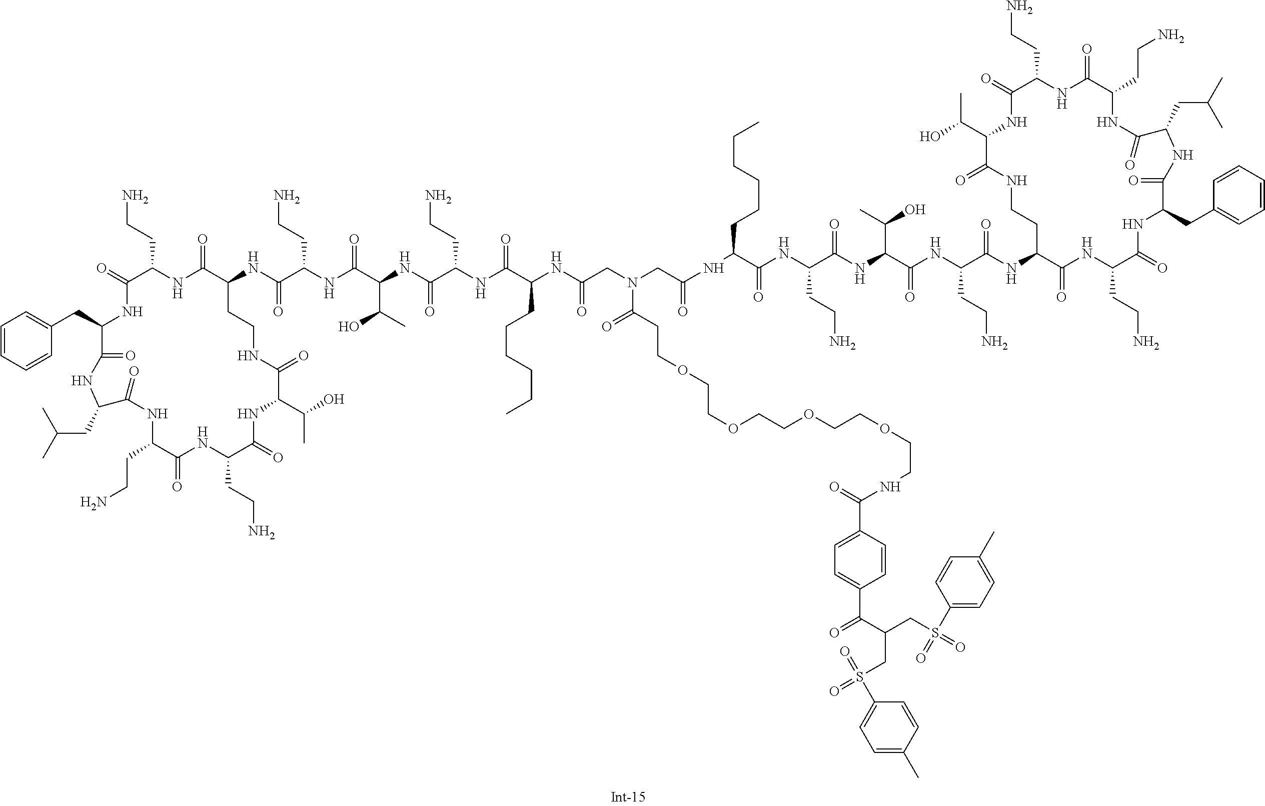

### Step a

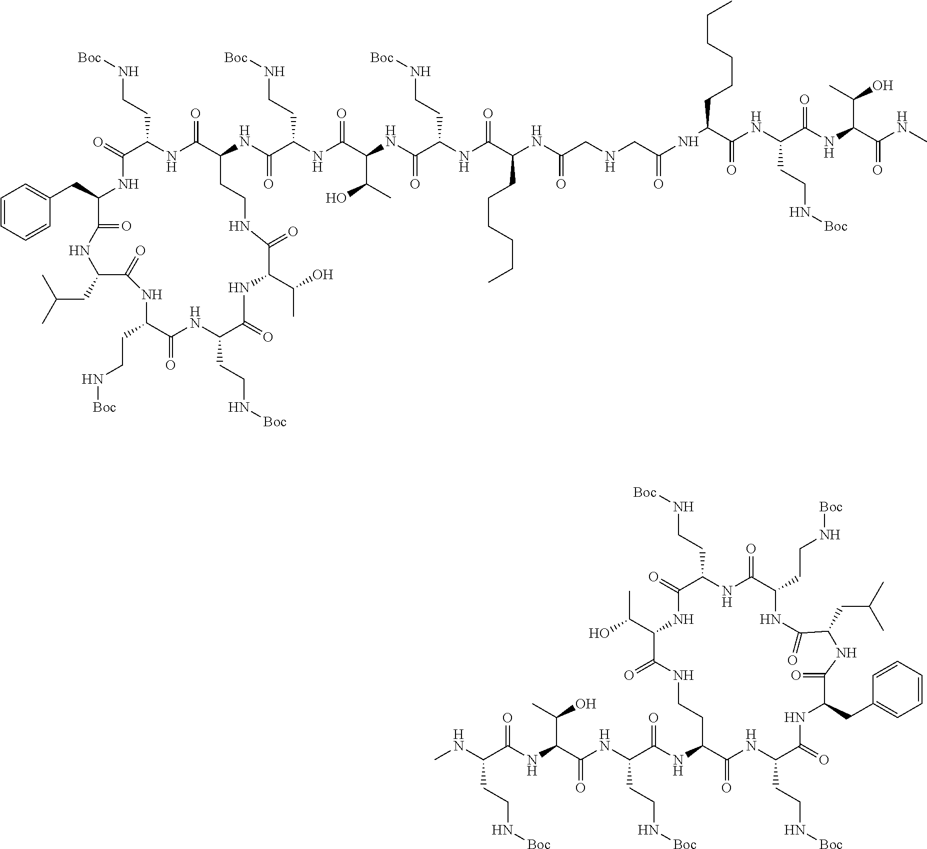

A solution of Cbz-iminodiacetic acid (0.149 g, 0.557 mmol), Int-18 (2.00 g, 1.173 mmol, described in Example 10 Step b), and DIEA (0.644 mL, 3.69 mmol) in DMF, was treated with a solution of HATU (0.468 g, 1.231 mmol) in DMF (5 mL), dropwise over 1 h. After stirring for an additional 30 minutes the reaction was charged with 5% Pd/C (1.0 g), vacuum flushed with hydrogen, and stirred under a hydrogen atmosphere for 2 h. The resulting mixture was filtered through celite, concentrated, and purified by RPLC using an Isco CombiFlash liquid chromatograph eluted with 30% to 100% methanol and water, using no modifier. Yield 1.0 g, 48% yield (two steps).

### Step b

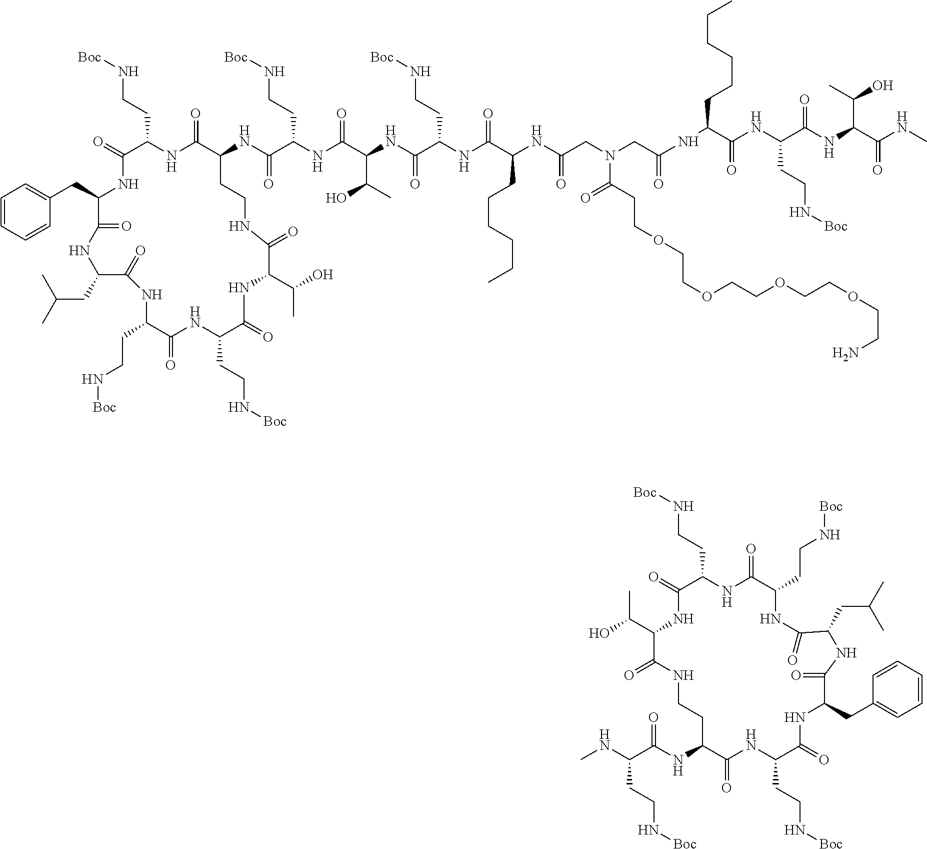

Product from Step a (1.03 g, 0.294 mmol), Cbz-N-amido-PEG4-acid (0.117 g, 0.294 mmol) and DIEA (0.110 mL, 0.617 mmol) in DMF (15 mL), was treated HATU (0.117 g, 0.308 mmol). After stirring for an additional 30 minutes the reaction was charged with 5% Pd/C (1.0 g), vacuum flushed with hydrogen, and stirred under a hydrogen atmosphere for 2 h. The resulting mixture was filtered through celite, concentrated, and purified by RPLC using an Isco CombiFlash liquid chromatograph eluted with 30% to 100% methanol and water, using no modifier. Yield 0.450 g, 41% yield (two steps).

### Step c

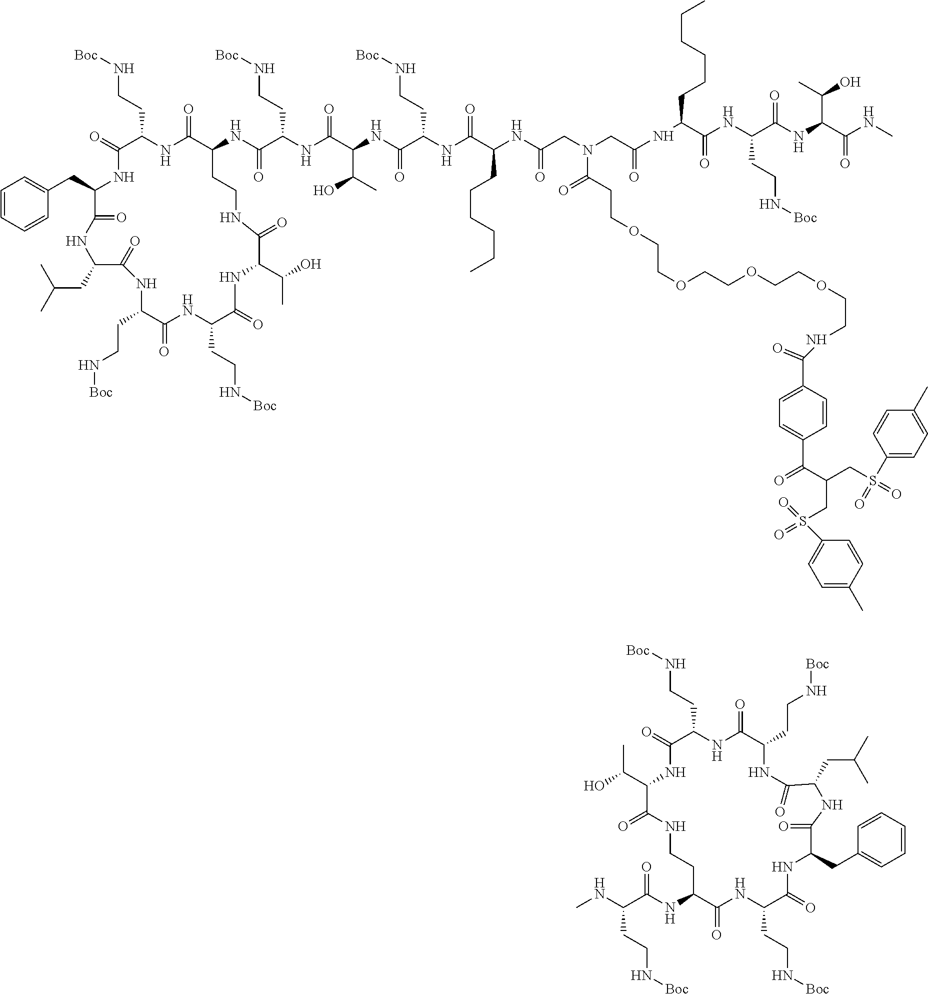

Product from Step b (0.450 g, 0.114 mmol), 4-(3-tosyl-2-(tosylmethyl)propanoyl)benzoic acid (0.023 g, 0.229 mmol) in DMF (10 mL), was treated HATU (0.043 g, 0.114 mmol). After stirring for 30 minutes the reaction was purified by RPLC using an Isco CombiFlash liquid chromatograph eluted with 30% to 100% methanol and water, using no modifier. Yield 0.158 g, 31% yield.

### Step d

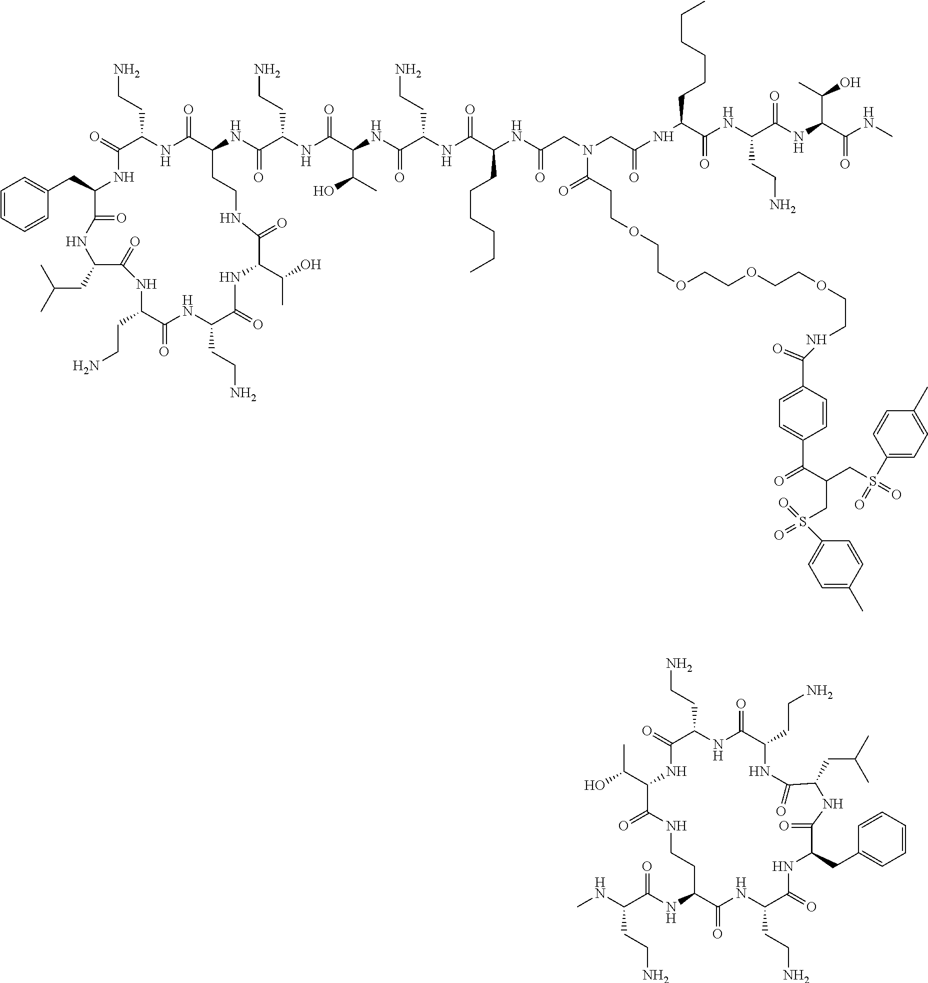

Product from Step c (0.158 g, 0.037 mmol) was treated with TFA (5 mL) for 5 minutes then concentrated to an oil. The oil was purified by RPLC using an Isco CombiFlash liquid chromatograph eluted with 10% to 100% methanol an water, using 0.1% TFA as the modifier. Yield 0.47 g, 27% yield.

### Example 35—Conjugate 18 (Conjugate of h-IgG1(His)₆ Fc-2 (Int-15))

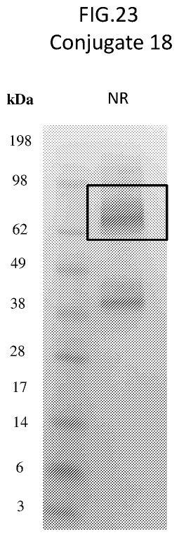

A solution of h-IgG1(His)₆ Fc (SEQ ID NO: 8) (857 NL, 10.20 mg, 0.1757 μmol, 11.90 mg/mL, MW=58,046 Da) in pH 7.4 PBS was treated with a water solution of TCEP (12.6 NL, 6.324 μmol, 500 mM) for 1 h, when Q-TOF analysis returned MW=28,773 Da. Excess TCEP was removed by buffer exchange with a 10,000 MWCO centrifugal filter (2×15 mL pH 7.4 PBS). The resulting solution was treated with Int-15 (6.15 mg, 1.405 μmol, 8 eq), then agitated gently for 1.5 h, when Q-TOF analysis showed full disappearance of the starting protein. Freshly prepared DTT water solution (54 μL, 3.513 μmol, 1 mg/100 μL) was added. The crude mixture was diluted 1:10 in PBS pH 7.4, and purified using MabSelect Sure Resin (GE Healthcare, Chicago, Ill., USA), followed by size exclusion chromatography. Purified material was quantified using a Nanodrop™ UV visible spectrophotometer (using a calculated extinction coefficient based on the amino acid sequence of h-IgG1), and concentrated to approximately 10 mg/mL using a centrifugal concentrator (10,000 MWCO). Purified molecules were analyzed using 4-12% Bis Tris SDS PAGE gels by loading 1-2 μg of each molecule into the gel, and staining using instant Blue staining. Each gel included a molecular weight ladder with the indicated molecular weight standards (FIG. 23). “NR” indicates non-reduced SDS-PAGE gel condition. Q-TOF analysis of the purified final product gave an average mass of 34,720 Da (DAR=4). Yield=9.38 mg, 77% yield.

### Example 36—Conjugate 19 (Conjugate of m-IgG2a(His). Fc-3 (Int-15))

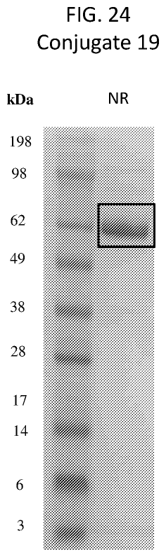

A solution of mIgG2a(His)₆ Fc (SEQ ID NO: 6) (900 μL, 8.69 mg, 0.1408 μmol, 9.658 mg/mL, MW=61,739 Da) in pH 7.4 PBS was treated with a water solution of TCEP (7.6 μL, 3.802 μmol, 500 mM) for 1 h, when Q-TOF analysis returned MW=29,998 Da. Excess of TCEP was removed by buffer exchange with a 10,000 MWCO centrifugal filter (2×15 mL pH 7.4 PBS). The resulting solution was treated with Int-15 (3.70 mg, 0.8447 μmol, 6 eq), then agitated gently for 1.5 h, when Q-TOF analysis showed full disappearance of the starting protein. Freshly prepared DTT water solution (43 μL, 2.816 μmol, 1 mg/100 μL) was added. The crude mixture was diluted 1:10 in PBS pH 7.4, and purified using MabSelect Sure Resin (GE Healthcare, Chicago, Ill., USA), followed by size exclusion chromatography. Purified material was quantified using a Nanodrop™ UV visible spectrophotometer (using a calculated extinction coefficient based on the amino acid sequence of m-IgG2a), and concentrated to approximately 10 mg/mL using a centrifugal concentrator (10,000 MWCO). Purified molecules were analyzed using 4-12% Bis Tris SDS PAGE gels by loading 1-2 μg of each molecule into the gel, and staining using instant Blue staining. Each gel included a molecular weight ladder with the indicated molecular weight standards (FIG. 24). Q-TOF analysis of the purified final product gave an average mass of 36,013 Da (DAR=4). Yield=8.69 mg, 86% yield.

### Example 37—Synthesis of Int-16

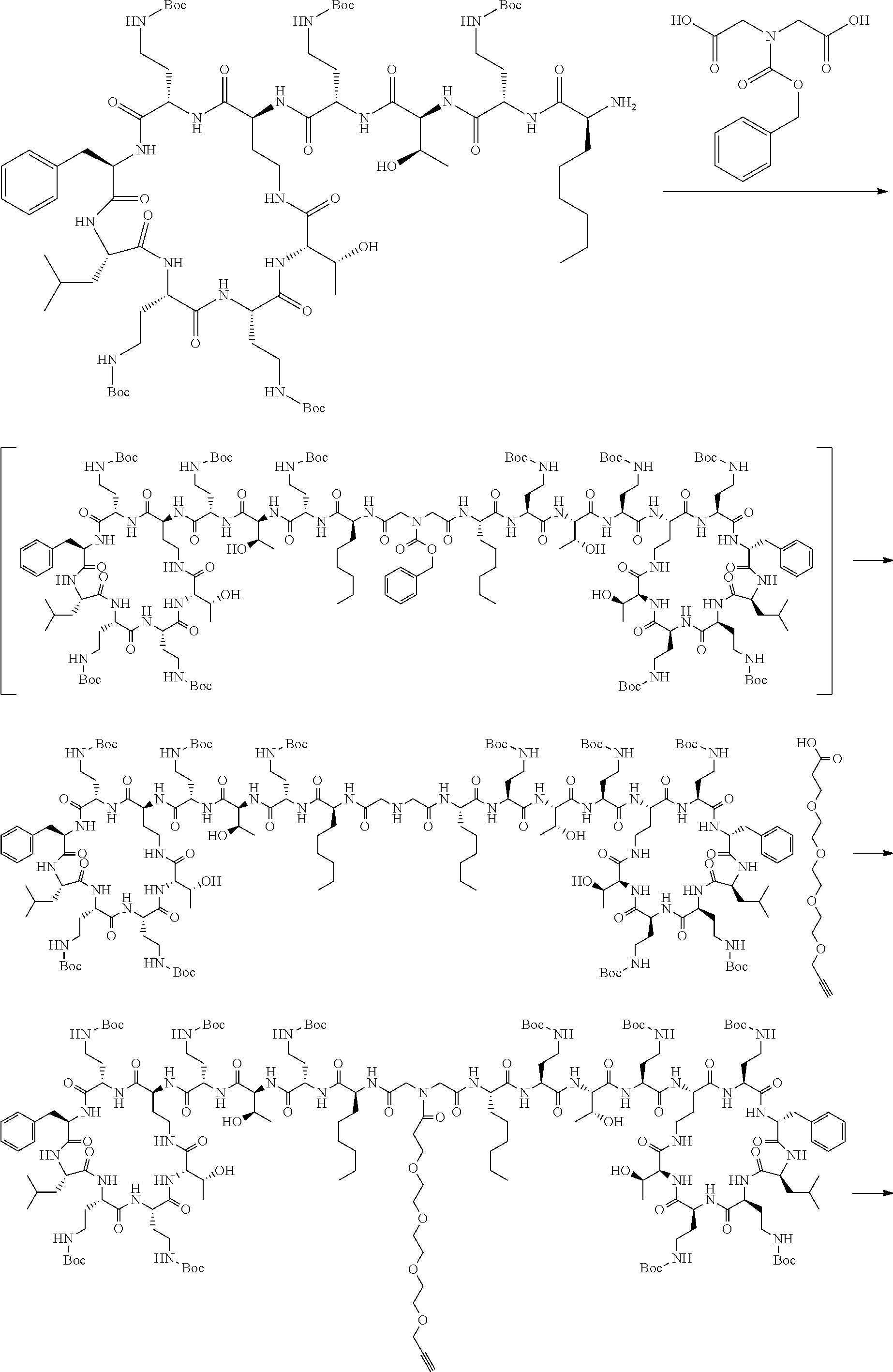

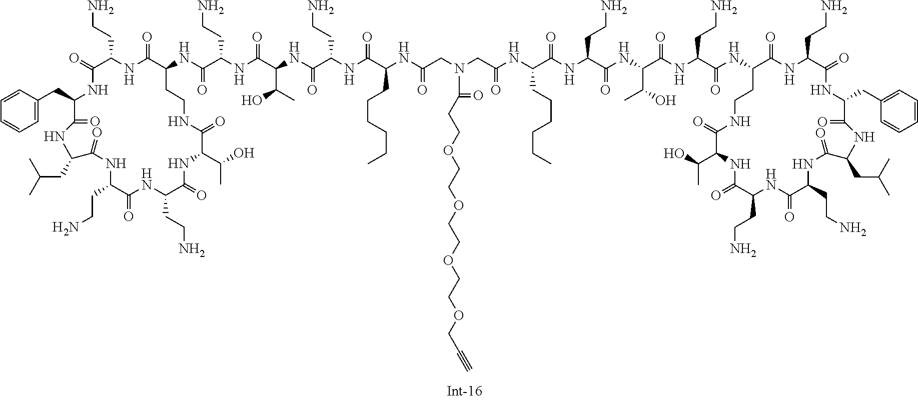 [SMILES: C#CCOCCOCCOCCOCCC(=O)N(CC(=O)N[C@@H](CCCCCC)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O)CC(=O)N[C@@H](CCCCCC)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O]

### Step a

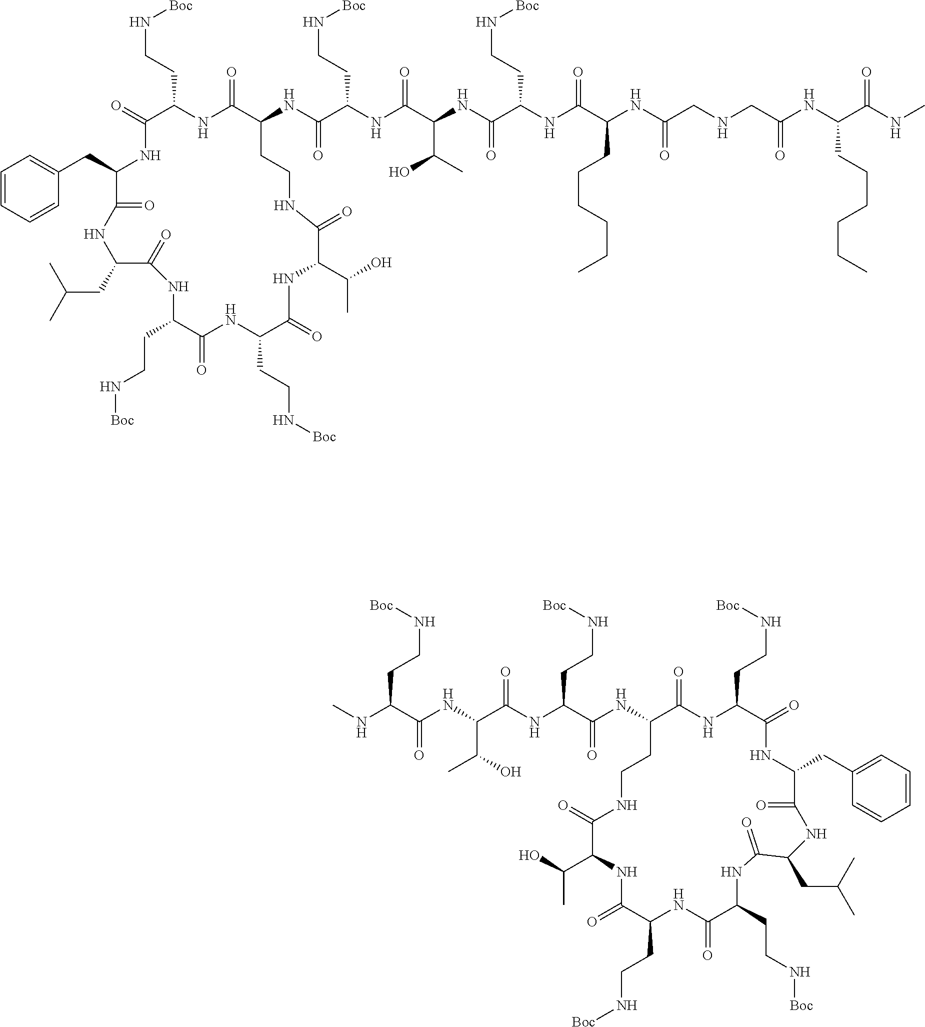

A solution of Cbz-iminodiacetic acid (0.149 g, 0.557 mmol), Int-18 (2.00 g, 1.173 mmol, described in Example 10 Step b), and DIEA (0.644 mL, 3.69 mmol) in DMF, was treated with a solution of HATU (0.458 g, 1.231 mmol) in DMF (5 mL), dropwise over 1 h. After stirring for an additional 30 minutes the reaction was charged with 5% Pd/C (1.0 g), vacuum flushed with hydrogen, and stirred under a hydrogen atmosphere for 2 h. The resulting mixture was filtered through celite, concentrated, and purified by RPLC using an Isco CombiFlash liquid chromatograph eluted with 30% to 100% methanol and water, using no modifier. Yield 1.0 g, 48% yield (two steps).

### Step h

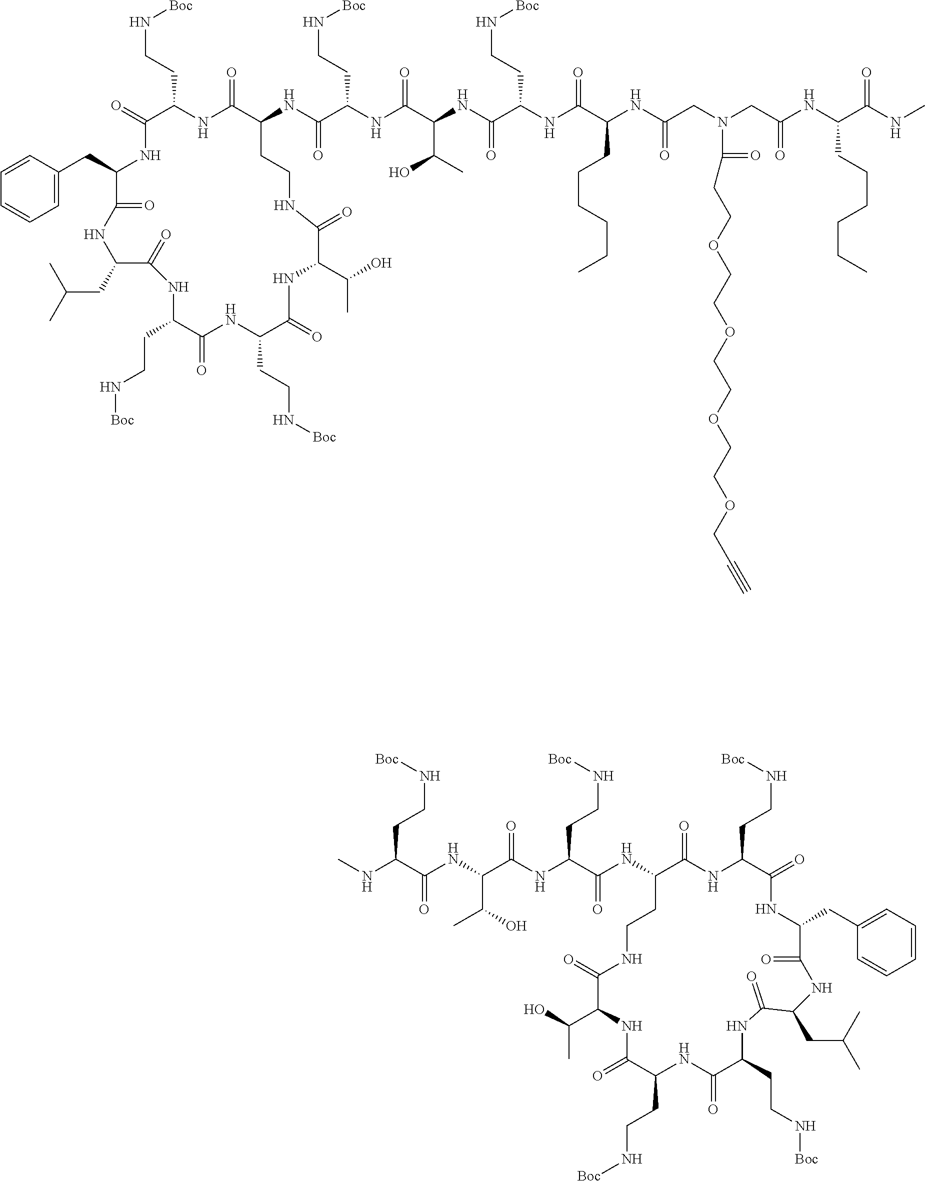

Product from Step a (0.800 g, 0.228 mmol), alkyne-peg4-acid (0.0833 g, 0.274 mmol) and DIEA (0.131 mL, 0.753 mmol) in DMF (5 mL), was treated HATU (0.0954 g, 0.251 mmol). After stirring for 30 minutes the reaction was purified by RPLC using an Isco CombiFlash liquid chromatograph eluted with 30% to 100% methanol and water, using no modifier. Yield 0.859 g, 99% yield.

### Step c

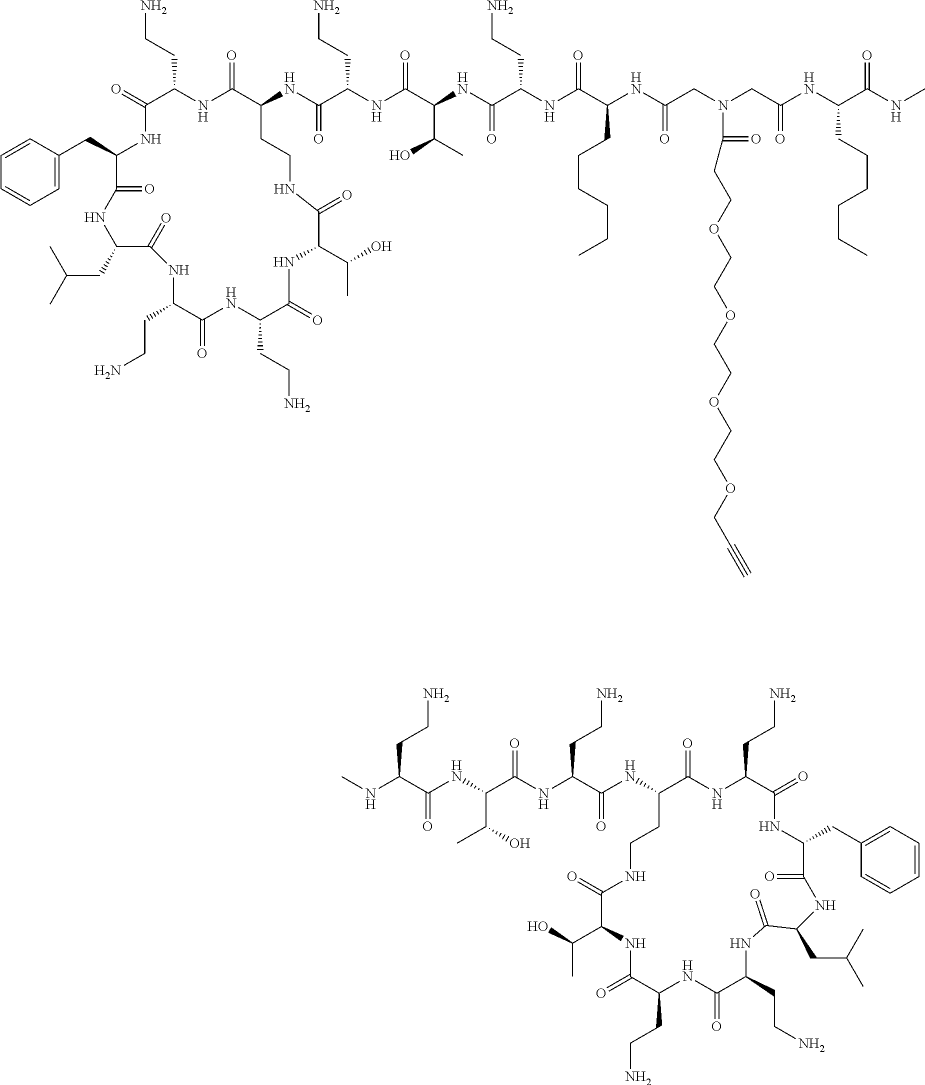

Product from Step b (0.859 g, 0.229 mmol) was treated with TFA (5 mL) for 5 minutes then concentrated to an oil. The oil was purified by RPLC using an Isco CombiFlash liquid chromatograph eluted with 10% to 100% methanol and water, using 0.1% TFA as the modifier. Yield 0.863 g, 97% yield.

### Example 36—Synthesis of Int-17

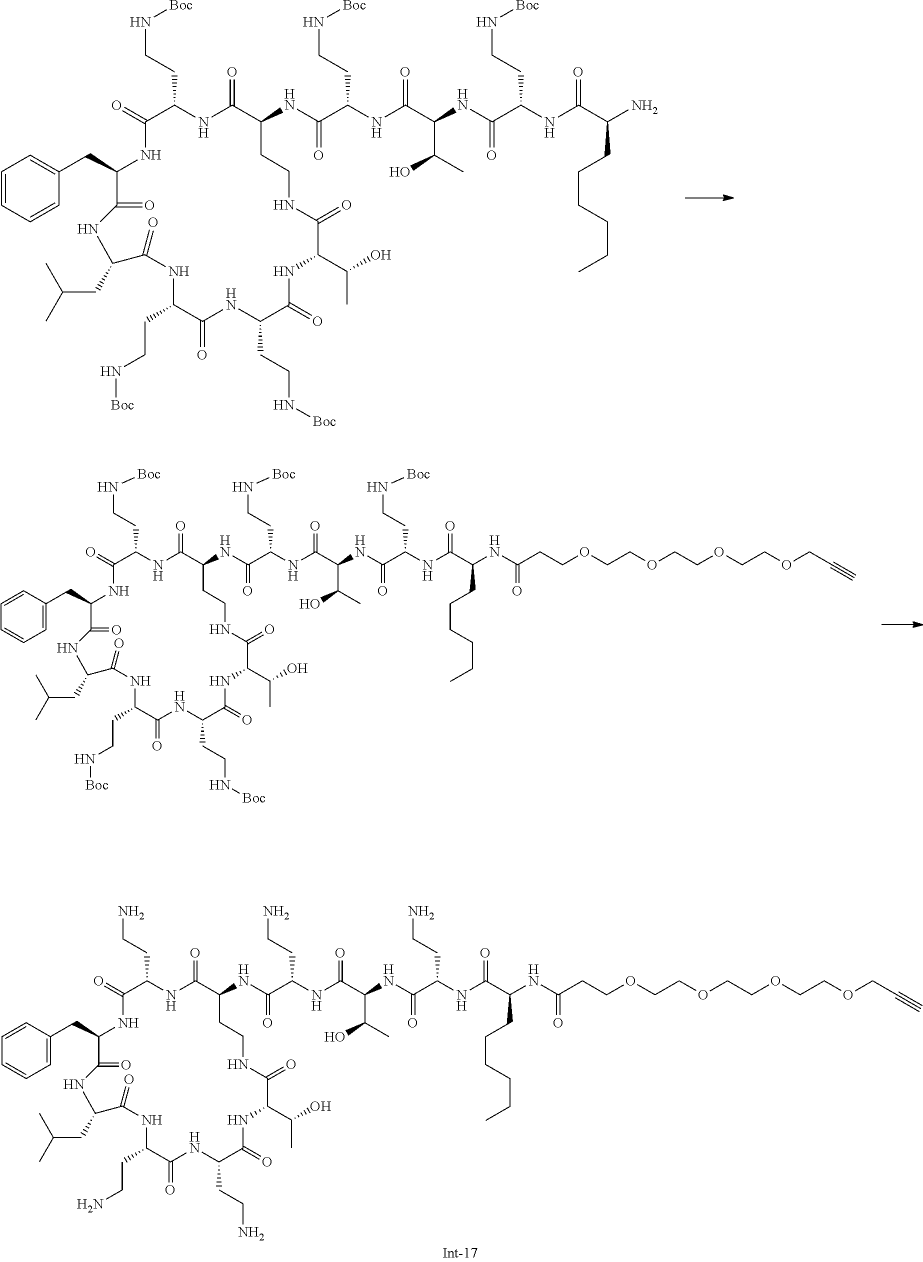

### Step a

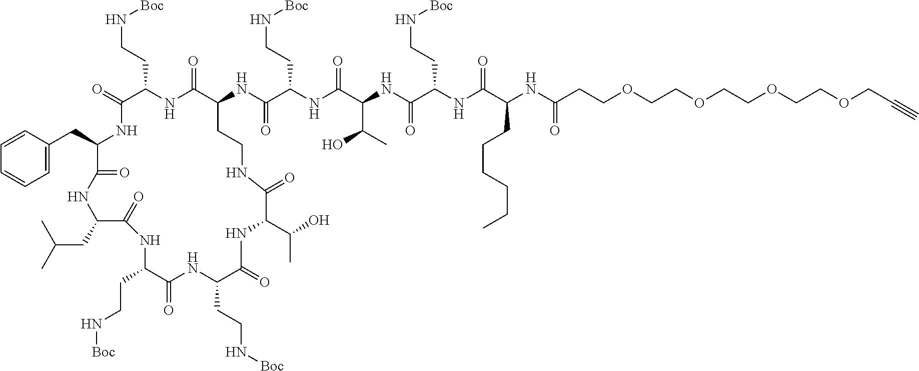 [SMILES: C#CCOCCOCCOCCOCCC(=O)N[C@@H](CCCCCC)C(=O)N[C@@H](CCNC(=O)OC(C)(C)C)C(=O)N[C@H](C(=O)N[C@@H](CCNC(=O)OC(C)(C)C)C(=O)N[C@H]1CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](Cc2ccccc2)NC(=O)[C@H](CCNC(=O)OC(C)(C)C)NC1=O)[C@@H](C)O]

A solution of Int-18 (0.800 g, 0.469 mmol, described in Example 10 Step b), alkyne-peg4 acid (0.134 g, 0.516 mmol), and DIEA (0.294 mL, 1.69 mmol) in DMF (3 mL), was treated with HATU (0.214 g, 0.583 mmol). After stirring for 30 minutes the reaction was purified by RPLC using an Isco CombiFlash liquid chromatograph eluted with 30% to 100% methanol and water, using no modifier. Yield 0.83 g, 91% yield.

### Step b

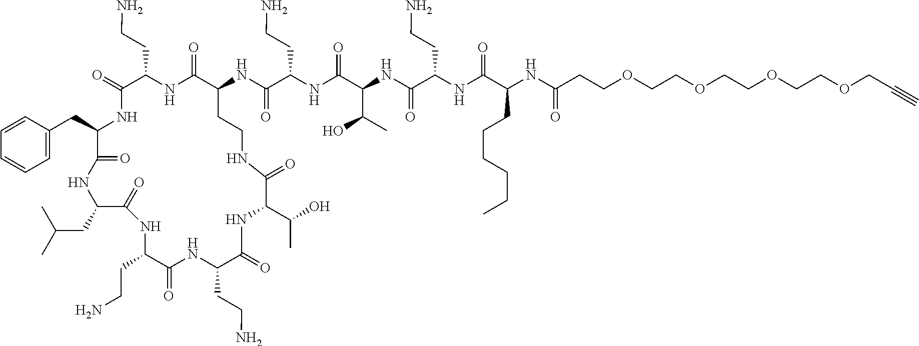 [SMILES: C#CCOCCOCCOCCOCCC(=O)N[C@@H](CCCCCC)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O]

A solution of penta Boc product from the previous Step (0.830 g, 0.426 mmol), was treated with TFA (5 mL). After stirring for 5 minutes the reaction was concentrated and purified by RPLC using an Isco CombiFlash liquid chromatograph eluted with 10% to 100% methanol and water, using TFA as the modifier. Yield 0.689 g, 80% yield.

### Example 39—Conjugate 20 (Conjugate of m-IgG2a(His)₆ Fc-3 (Int-16))

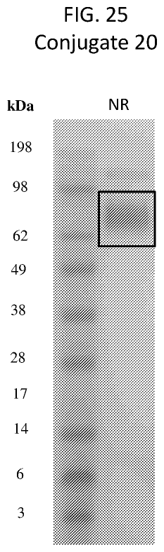

A solution of mIgG2a(His)₆ Fc (SEQ ID NO: 6) (42.6 mg, 0.0707 μmol, 10.65 mg/mL, MW=60,252da) in pH7.4 PBS was treated with azido-peg4-NHS ester (32.9 mg, 84.8 μmol, 120 eq), then agitated gently overnight at RT. Unreacted azido-peg4-NHS ester was removed by buffer exchange with a 10,000 MWCO centrifugal filter (6×15 mL pH 7.4 PBS). The final volume of the azide functionalized Fc was 3.0 mL. Maldi TOF analysis gave a molecular weight of 64,396 (azide DAR=15.1). To this was added freshly prepared solutions of CuSO₄ (300 μL of 20 mM), THPTA (300 μL of 50 mM, Tris(3-hydroxypropyltriazolylmethyl)amine), ascorbic acid (300 μL of 100 mM), and alkyne derivatized small molecule (109.9 mg, 28.3 μmol, Int-16)). The crude mixture was diluted 1:10 in PBS pH 7.4, and purified using MabSelect Sure Resin (GE Healthcare, Chicago, Ill., USA), followed by size exclusion chromatography. Purified material was quantified using a Nanodrop™ UV visible spectrophotometer (using a calculated extinction coefficient based on the amino acid sequence of m-IgG2a), and concentrated to approximately 10 mg/mL using a centrifugal concentrator (10,000 MWCO). Purified molecules were analyzed using 4-12% Bis Tris SDS PAGE gels by loading 1-2 μg of each molecule into the gel, and staining using instant Blue staining. Each gel included a molecular weight ladder with the indicated molecular weight standards (FIG. 25). Maldi TOF analysis of the purified final product gave an average mass of 86,577 Da (DAR=10). Yield=72 mg, 70% yield.

### Example 40—Conjugate 21 (Conjugate of h-IgG1(His)₆ Fc-2 (Int-17))

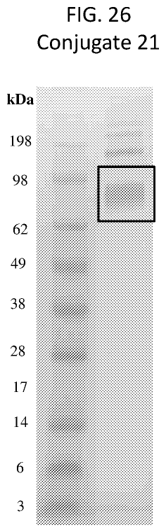

A solution of hIgG1(His)₆ Fc (SEQ ID NO: 8) (67.2 mg, 1.19 μmol, 16.8 mg/mL, MW=56,633 Da) in pH7.4 PBS was treated with azido-peg4-NHS ester (55.0 mg, 142.5 μmol, 120 eq), then agitated gently overnight at RT. Unreacted azido-peg4-NHS ester was removed by buffer exchange with a 10,000 MWCO centrifugal filter (6×15 mL pH 7.4 PBS). The final volume of the azide functionalized Fc was 5.0 mL. To this was added freshly prepared solutions of CuSO₄ (500 μL of 20 mM), THPTA (500 μL of 50 mM, Tris(3-hydroxypropyltriazolylmethyl)amine), ascorbic acid (500 μL of 100 mM), and alkyne derivatized small molecule (95.7 mg, 47.5 μmol, Int-17). The resulting solution was agitated gently overnight. The crude mixture was diluted 1:10 in PBS pH 7.4, and purified using MabSelect Sure Resin (GE Healthcare, Chicago, Ill., USA), followed by size exclusion chromatography. Purified material was quantified using a Nanodrop™ UV visible spectrophotometer (using a calculated extinction coefficient based on the amino acid sequence of h-IgG1), and concentrated to approximately 10 mg/mL using a centrifugal concentrator (10,000 MWCO). Purified molecules were analyzed using 4-12% Bis Tris SDS PAGE gels by loading 1-2 μg of each molecule into the gel, and staining using instant Blue staining. Each gel included a molecular weight ladder with the indicated molecular weight standards (FIG. 26). “NR” indicates non-reduced SDS-PAGE gel condition. Maldi TOF analysis of the purified final product gave a mass of an average mass of 86,892 Da (DAR=15). Yield 67 mg, 77% yield.

### Example 41—Synthesis of Int-19, Int-20, Int-21 and Int-22

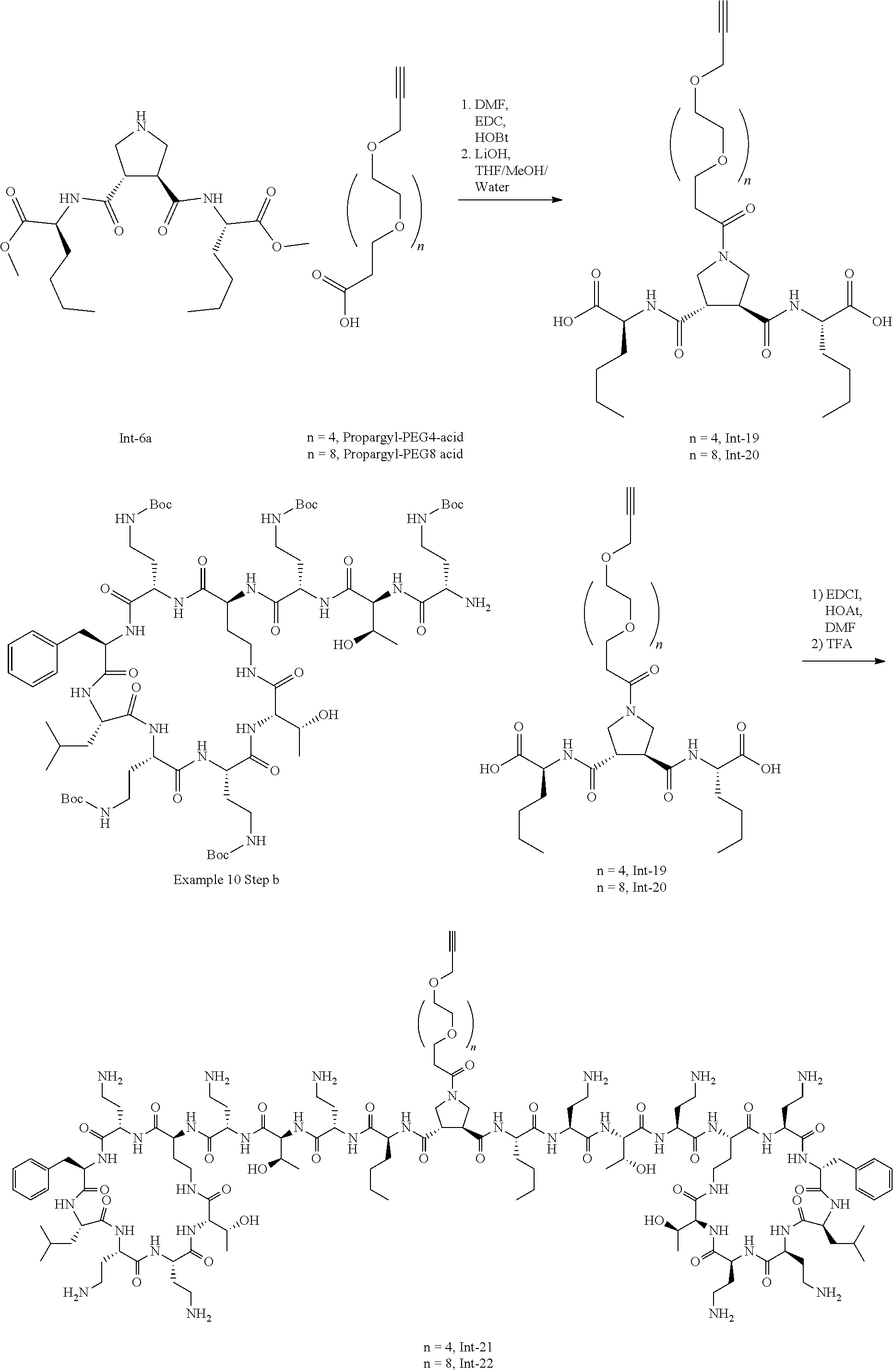

### Synthesis of Int-19 and Int-20

HATU (198 mg, 0.52 mmol, in 1 mL of DMF) was added to a stirring mixture of propargyl-PEG4-acid (148 mg, 0.57 mmol), Int-6a, TFA salt (250 mg, 0.47 mmol) and triethylamine (158 mg, 1.57 mmol) in DMF (2 mL) and the mixture was stirred at ambient temperature for 30 minutes. The mixture was applied directly to RPLC using an Isco Combiflash liquid chromatograph eluted with 10% to 95% acetonitrile and water using 0.1% TFA modifier. The pure fractions were pooled and lyophilized to afford the di-ester intermediate. LC/MS [M+H]+=656.4. The di-ester intermediate was stirred in a 1/1/2 mixture of MeOH/THF/DI water containing LiOH (36 mg, 1.5 mmol) at ambient temperature for 5 minutes. The mixture was acidified with glacial acetic acid, concentrated and applied directly to RPLC using an Isco Combiflash liquid chromatograph eluted with 10% to 95% acetonitrile and water using 0.1% TFA modifier. The pure fractions were pooled and lyophilized to afford the title compound Int-19 as a clear oil. Yield: 74%, 2 Steps. Ion found by LC/MS [M−H]−=626.2.

Int-20 was synthesized in a similar way as described for Int-19. Isolated yield was 73%. Positive mass ions were found by LC/MS at tr=4.10 with 8-minute 5˜95% CAN/water method: (M+2H⁺)/2=402.5.

### Synthesis of Int-21

A solid mixture of PMBD (example 10 Step b, 7.444 g, 4.760 mmol), Int-19 1.494 g, 2.380 mmol), EDCI (1.141 g, 5.950 mmol) and HOAt (0.8099 g, 5.950 mmol) were dissolved in 20 mL of dry DMF immediately followed by adding triethylamine (1.66 mL, 9.52 mmol). The mixture was stirred for overnight (˜15 hours) at room temperature. HPLC analysis showed the completion of the reaction. Most of the solvent (DMF) of the reaction mixture was removed by reduced pressure rotovap to give a viscous oily residue. To this residue methanol was added with gentle stirring to triturate the reaction product. After some crystalline solid started to precipitate the flask was put standstill for more than 3 hours. Simple filtration collected the crystalline white solid with methanol wash (3×5 mL). The solid was dried to give 6.500 grams of pure product (73% isolated yield). The solid was dissolved with 100% TFA and stirred for 30 minutes. TFA was removed by rotovap. The residue was dissolved in minimum amount of water and purified by RPLC using an Isco Combiflash liquid chromatograph eluted with 5% to 35% acetonitrile and water using 0.1% TFA modifier. The pure fractions were lyophilized to afford the title product Int-21 as a white powder (TFA salt, 6.08 g, 90% isolated yield, Positive mass ions were found (M+6H⁺)/6=453.8 and (M+5H⁺)/5=544.2.

### Synthesis of Int-22

A solid mixture of PMBD (example 10 Step b, 2.505 g, 1.602 mmol), Int-20 (0.644 g, 0.801 mmol), EDCI (0.3839 g, 2.003 mmol) and HOAt (0.0.2726 g, 2.003 mmol) were dissolved in 20 mL of dry DMF immediately followed by adding triethylamine (0.56 mL, 3.2 mmol). The mixture was stirred for overnight (˜15 hours) at room temperature. HPLC analysis showed the completion of the reaction. Most of the solvent (DMF) of the reaction mixture was removed by reduced pressure rotovap to give a viscous oily residue. To this residue methanol was added with gentle stirring to triturate the reaction product. After some crystalline solid started to precipitate the flask was put standstill for more than 3 hours. Simple filtration collected the crystalline white solid with methanol wash (3×5 mL). The solid was dried to give 2.183 grams of pure product (70% isolated yield). The solid (2.020 g) was dissolved with 100% TFA and stirred for 30 minutes. TFA was removed by rotovap. The residue was dissolved in minimum amount of water and purified by RPLC using an Isco Combiflash liquid chromatograph eluted with 5% to 35% acetonitrile and water using 0.1% TFA modifier. The pure fractions were lyophilized to afford the product Int-22 as a white powder (TFA salt, 2.297 g, 90% isolated yield,

Positive mass ions were found by LC/MS at tr=1.87 with 8-minute 5˜95% CAN/water method: (M+7H⁺)/7=414.2, (M+6H⁺)/6=483.0, (M+5H⁺)/5=579.5, (M+4H⁺)/4=724.1.

### Example 42—Conjugate 22 (Conjugate of h-IgG1(His)₆ Fc-2 (Int-21))

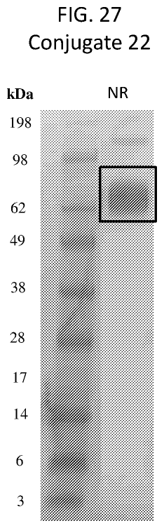

A solution of hIgG1(His)₆Fc (SEQ ID NO: 8) (15.0 mL, 2.54 μmol, 9.33 mg/mL, MW=55111 Da) in pH 7.4 PBS×1 was treated with azido-peg4-NHS ester (0.0384 g in 150 μL of DMF, 96.9 μmol, 38 eq), then agitated gently overnight at RT. Unreacted azido-peg4-NHS ester was removed by buffer exchange with a 10,000 MWCO centrifugal filter (6×15 mL pH 7.4 PBS×1). The final volume of the azide functionalized Fc was 9.00 mL with concentration at 5.16 mg/mL, MW=60302. To this Fc-azido PBS×1 buffer solution (0.0781 g, 1.50 μmol, 15.15 mL) were added alkyne derivatized small molecule (TFA salt, 209.7 mg, 54.4 μmol, Int-21) and freshly prepared pH 7.4 PBS×1 solutions of CuSO₄ (4.49 mL of 10.0 mM, 30 eq), tris(3-hydroxypropyltriazolylmethyl)-amine (THPTA, 4.49 mL of 10.0 mM, 30 eq), and ascorbic acid (4.49 mL of 10.0 mM, 30 eq). The resulting solution was agitated gently for 4 hours at room temperature. Another portion of freshly prepared pH 7.4 PBS×1 solutions of CuSO₄ (2.25 mL, of 10.0 mM, 15 eq) was added and the reaction mixture was gently agitated for overnight (˜15 hours) at room temperature, then the final portion of freshly prepared pH 7.4 PBS×1 solutions of CuSO₄ (2.25 mL, of 10.0 mM, 15 eq) was added and the reaction mixture was gently agitated for 2 hours. The crude mixture was diluted 1:10 in PBS pH 7.4, and purified using MabSelect Sure Resin (GE Healthcare, Chicago, Ill., USA), followed by size exclusion chromatography. Purified material was quantified using a Nanodrop™ UV visible spectrophotometer (using a calculated extinction coefficient based on the amino acid sequence of h-IgG1), and concentrated to approximately 10 mg/mL using a centrifugal concentrator (10,000 MWCO). Purified molecules were analyzed using 4-12% Bis Tris SDS PAGE gels by loading 1-2 μg of each molecule into the gel, and staining using instant Blue staining. Each gel included a molecular weight ladder with the indicated molecular weight standards (FIG. 27). “NR” indicates non-reduced SDS-PAGE gel condition. MALDI TOF analysis of the purified final product gave a mass of an average mass of 82535 Da (DAR=8). 89% yield.

### Example 43—Conjugate 23 (Conjugate of h-IgG1(c-myc) Fc-2 (Int-21))

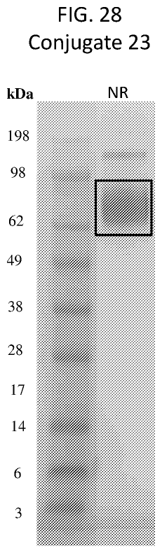

A solution of hIgG1(c-myc) Fc (SEQ ID NO: 17) (9.50 mL, 1.68 μmol, 10.3 mg/mL, MW=58,065 Da) in pH 7.4 PBS×1 was treated with azido-peg4-NHS ester (98%, 6.66 mg, 16.8 μmol, 10 eq), then agitated gently overnight at RT. Unreacted azido-peg4-NHS ester was removed by buffer exchange with a 10,000 MWCO centrifugal filter (6×15 mL pH 7.4 PBS×1). The final volume of the azide functionalized Fc was 9.00 mL with concentration at 8.91 mg/mL, MW=59006. To this Fc-azido PBS×1 buffer solution were added freshly prepared pH 7.4 PBS×1 solutions of CuSO₄ (2.04 mL of 10.0 mM, 15 eq), Tris(3-hydroxypropyltriazolylmethyl)-amine (THPTA, 2.04 mL of 10.0 mM, 15 eq), ascorbic acid (2.04 mL of 10.0 mM, 15 eq), and alkyne derivatized small molecule (TFA salt, 209.7 mg, 54.4 μmol, Int-21). The resulting solution was agitated gently for 4 hours at room temperature. Another portion of freshly prepared pH 7.4 PBS×1 solutions of CuSO₄ (1.02 mL, of 10.0 mM, 7.5 eq) was added and the reaction mixture was gently agitated for overnight (˜15 hours) at room temperature, then the final portion of freshly prepared pH 7.4 PBS×1 solutions of CuSO₄ (1.02 mL, of 10.0 mM, 7.5 eq) was added and the reaction mixture was gently agitated for 2 hours. The crude mixture was diluted 1:10 in PBS pH 7.4, and purified using MabSelect Sure Resin (GE Healthcare, Chicago, Ill., USA), followed by size exclusion chromatography. Purified material was quantified using a Nanodrop™ UV visible spectrophotometer (using a calculated extinction coefficient based on the amino acid sequence of h-IgG1), and concentrated to approximately 10 mg/mL using a centrifugal concentrator (10,000 MWCO). Purified molecules were analyzed using 4-12% Bis Tris SDS PAGE gels by loading 1-2 μg of each molecule into the gel, and staining using instant Blue staining. Each gel included a molecular weight ladder with the indicated molecular weight standards (FIG. 28). “NR” indicates non-reduced SDS-PAGE gel condition. MALDI TOF analysis of the purified final product gave a mass of an average mass of 69010 Da (DAR=3.7). 75% yield.

### Example 44—Synthesis of Int-23

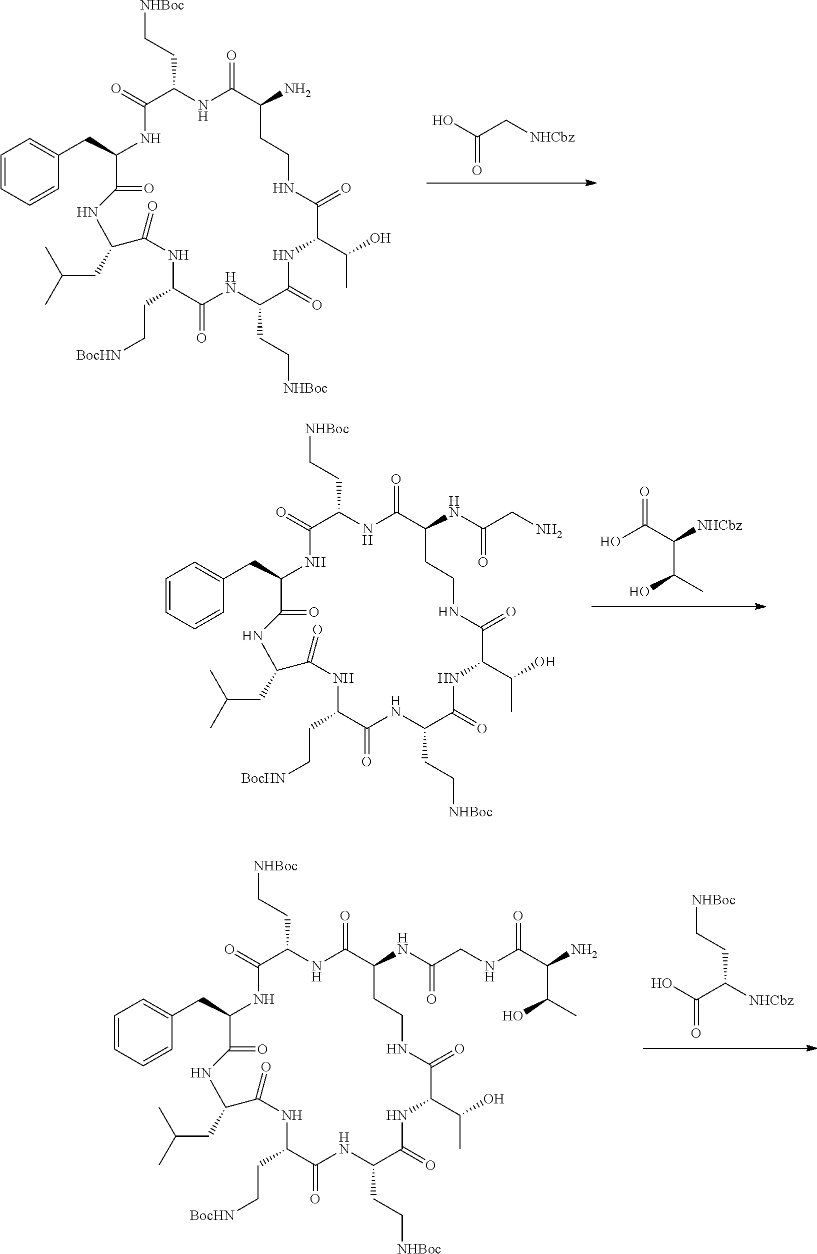

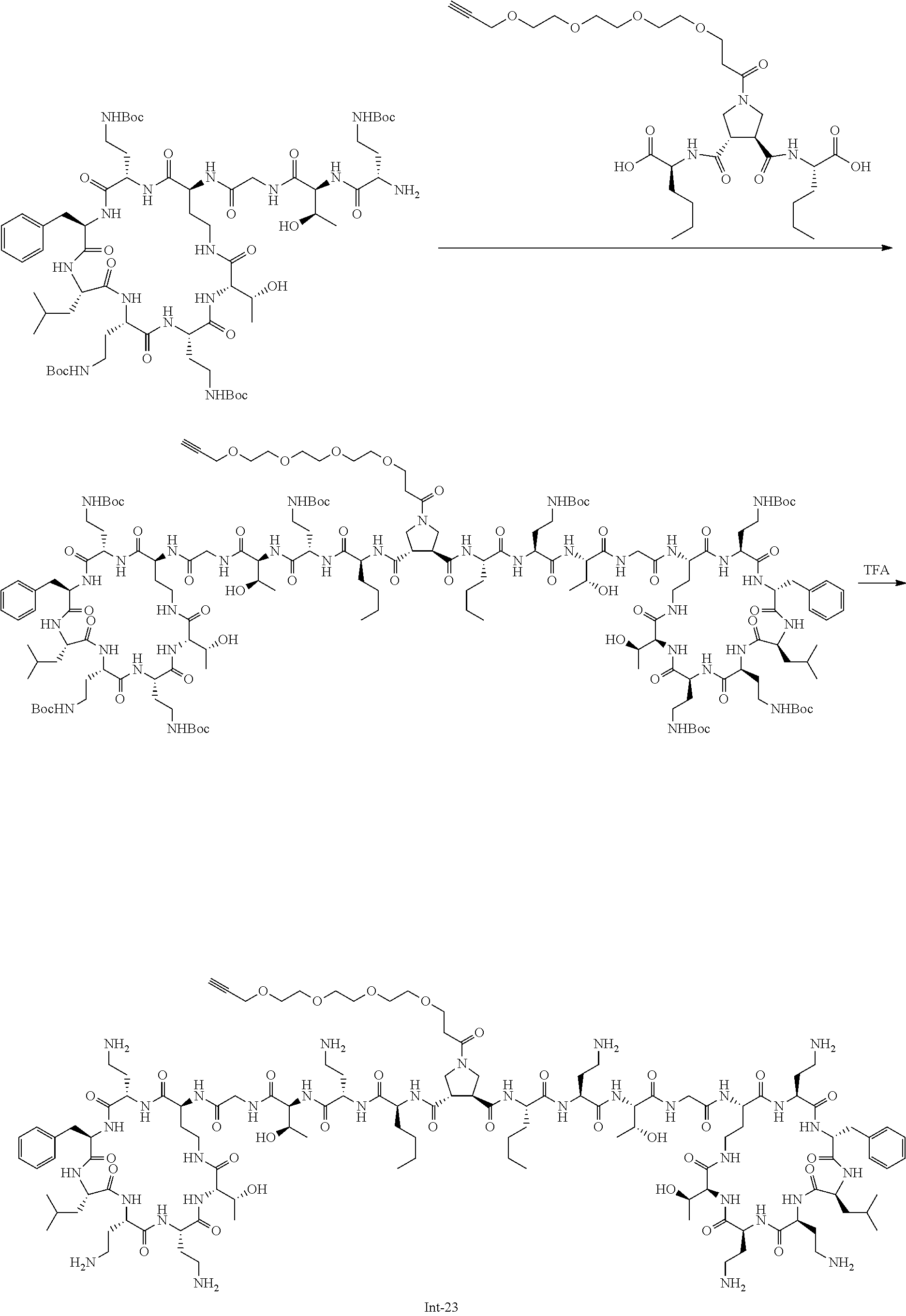

### Step a. Synthesis of Int-1-Gly Conjugate

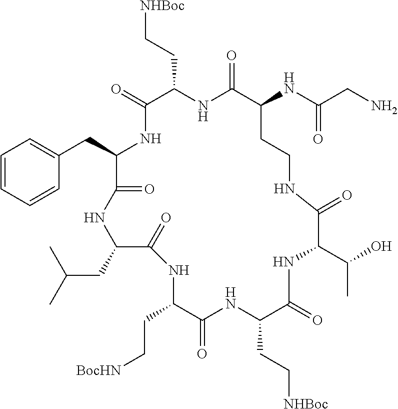 [SMILES: CC(C)C[C@@H]1NC(=O)[C@@H](Cc2ccccc2)NC(=O)[C@H](CCNC(=O)OC(C)(C)C)NC(=O)[C@@H](NC(=O)CN)CCNC(=O)[C@H]([C@@H](C)O)NC(=O)[C@H](CCNC(=O)OC(C)(C)C)NC(=O)[C@H](CCNC(=O)OC(C)(C)C)NC1=O]

To a mixture of Int-1 (4.24 g, 4 mmol) and Z-Gly-OH (1 g, 4.8 mmol) in anhydrous DMF (8 mL) was added HATU (1.87 g, 4.9 mmol) in portions over 20 minutes, followed by DIPEA (936 mg, 7.2 mmol). After the reaction mixture was stirred for 15 minutes, it was poured into water (100 mL). The white solid product was collected by filtration and washed with water. The material was re-dissolved in MeOH (50 mL) and treated with Pd/C (1 g). The mixture was stirred under hydrogen overnight. Pd/C was then filtered off, and the filtrate was concentrated and purified through RPLC (150 g, 15 to 75% MeOH and water). Yield 4.02 g, 89.8%. Ion found by LCMS: [(M−Boc+2H)/2]⁺=510.4, [(M−3Boc+2H)/2]⁺=410.2

### Step b

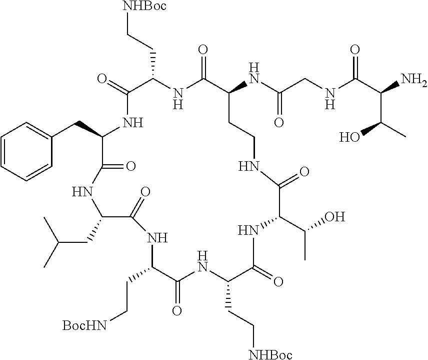 [SMILES: CC(C)C[C@@H]1NC(=O)[C@@H](Cc2ccccc2)NC(=O)[C@H](CCNC(=O)OC(C)(C)C)NC(=O)[C@@H](NC(=O)CNC(=O)[C@@H](N)[C@@H](C)O)CCNC(=O)[C@H]([C@@H](C)O)NC(=O)[C@H](CCNC(=O)OC(C)(C)C)NC(=O)[C@H](CCNC(=O)OC(C)(C)C)NC1=O]

To a mixture of the Step-a product (4.02 g, 3.592 mmol) and Z-Thr-OH (980.3 mg, 3.87 mmol) in anhydrous DMF (5 mL) was added HATU (1.47 g, 3.87 mmol) in portions over 10 minutes, followed by DIPEA (755 mg, 5.8 mmol). After the addition, the reaction was stirred for 20 minutes and then poured into water (100 mL). The white solid product was collected by filtration and washed with water. The material was re-dissolved in MeOH (50 mL) and added with Pd/C (1 g). The mixture was stirred under hydrogen overnight. Pd/C was then filtered, and the filtrate was concentrated and purified through RPLC (150 g, 15 to 80% MeOH and water). Yield 3.68 g, 83.9%. Ions found by LCMS: [(M−2Boc+2H)/2]⁺=511.

### Step c

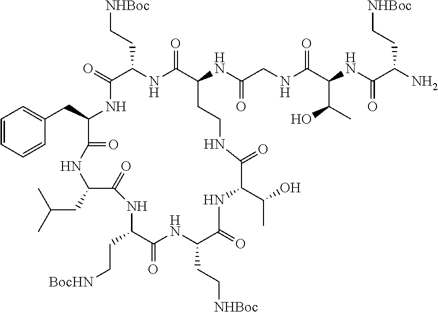 [SMILES: CC(C)C[C@@H]1NC(=O)[C@@H](Cc2ccccc2)NC(=O)[C@H](CCNC(=O)OC(C)(C)C)NC(=O)[C@@H](NC(=O)CNC(=O)[C@@H](NC(=O)[C@@H](N)CCNC(=O)OC(C)(C)C)[C@@H](C)O)CCNC(=O)[C@H]([C@@H](C)O)NC(=O)[C@H](CCNC(=O)OC(C)(C)C)NC(=O)[C@H](CCNC(=O)OC(C)(C)C)NC1=O]

A mixture of the Step-b product (1.2 g, 0.984 mmol) and Z-Dab(Boc)-OH.DCHA (605 mg, 1.13 mmol) was dissolved in anhydrous NMP (3 mL). It was added with HATU (430 mg, 1.13 mmol) in portions over 5 minutes, followed by DIPEA (150 mg, 1.13 mmol). The reaction was stirred for 30 minutes and then directly purified through RPLC (100 g, 40 to 100% MeOH and water). The collected fractions were concentrated by rotary evaporation to a white solid (Ion found by LCMS: [M−2Boc+2H)/2]⁺=678). The material was re-dissolved in MeOH (30 mL) and treated with Pd/C. The mixture was stirred under hydrogen overnight. Pd/C was filtered, and the filtrate was concentrated by rotary evaporation and further dried under high vacuum. Yield 1.07 g, 76.6%. Ion found by LCMS: [(M−2Boc+2H)/2]⁺=611.

### Step d. Synthesis of Octa-Boc Int-23

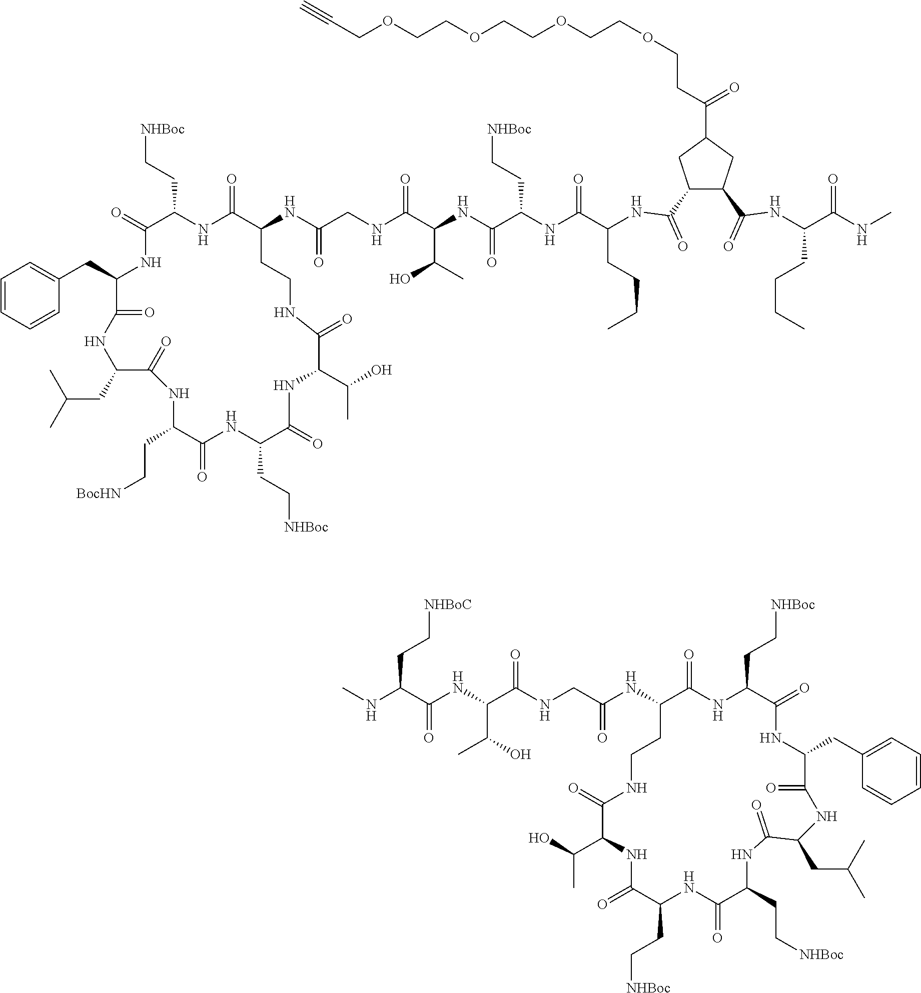

A mixture of the Step-c product (313.6 mg, 0.2207 mmol) and INT-8 alkyne-PEG4-Pyrrolidine-(NLeu-CO2H)2 (65.9 mg, 0.105 mmol) was dissolved in anhydrous DMF (1 mL). It was added with DIPEA (65 mg, 0.5 mmol) followed by HATU (83.9 mg, 0.2207 mmol) in portions over 5 minutes. The reaction was stirred for 30 minutes and then directly purified through RPLC (50 g, 40 to 100% MeOH and water). Yield 165 mg, 49.6%. Ions found by LCMS: [M−3Boc+3H)/3]⁺=1044.6, [M−4Boc+3H)/3]⁺=1011.3, [M−6Boc+3H)/3]⁺=944.4.

### Step e. Synthesis of Int-23

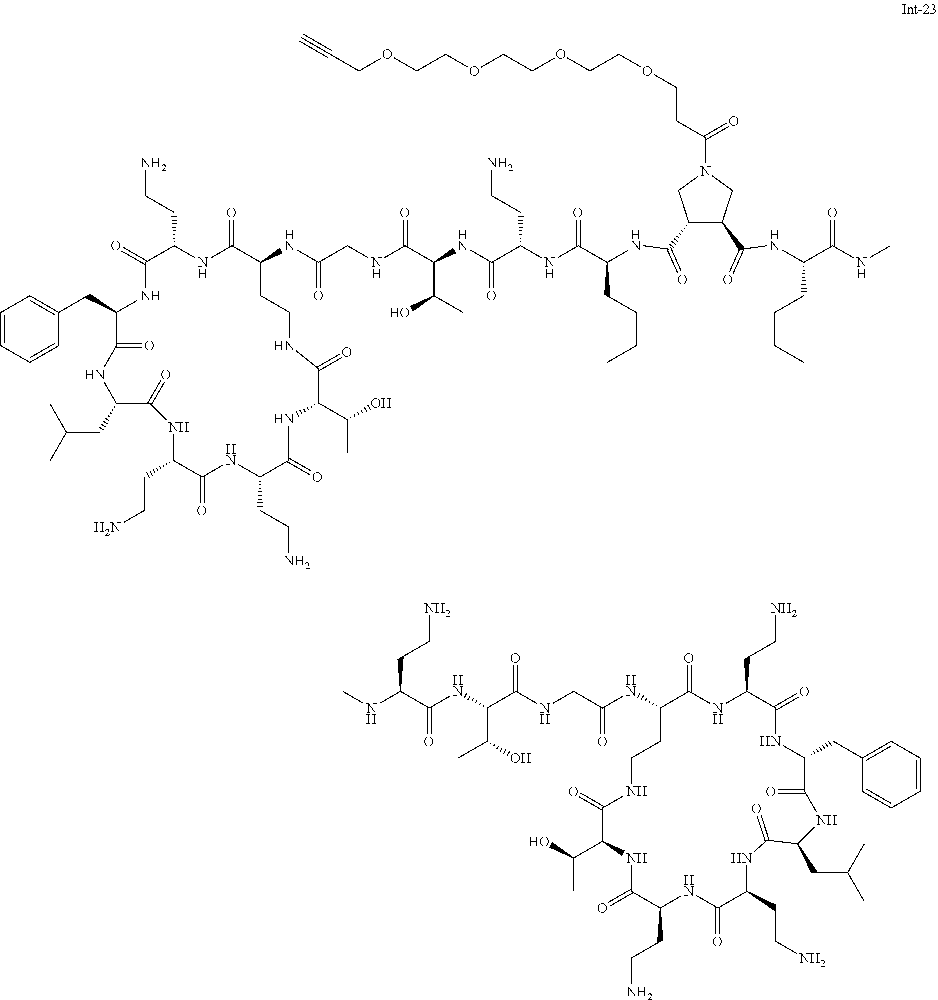

The Step-d product (165 mg, 0.0521 mmol) was dissolved in TFA/DCM (1:1, 1 mL), and the solution was stirred for 30 minutes. It was directly purified by RPLC (50 g, 5 to 30% acetonitrile and water, using 0.1% TFA as modifier). Yield 160 mg, 86.6%. Ions found by LCMS: [M+4H)/4]⁺=658.4. [M+5H)/5]⁺=527.1, [M+6H)/6]⁺=439.4, [M+7H)/7]⁺=376.8.

### Example 45—Conjugate 24 (Conjugate of h-IgG1(c-myc) Fc-2 (Int-23))

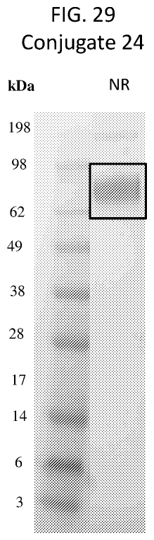

Int-21 (60 mg, 0.0169 mmol) was added to h-IgG1(c-myc) Fc (SEQ ID NO: 17)-(azido)5 conjugate (0.0002079 M, 2 mL) solution, followed by 0.832 mL of 0.01 M CuSO₄ solution, 0.832 mL of 0.01 M sodium ascorbate solution, and 0.832 mL of 0.01 M THPTA solution. The reaction was gently shaken at room temperature of 4 hours, then added with an additional amount of freshly prepared CuSO₄ solution (0.01M, 0.416 mL). The reaction was shaken overnight. Additional CuSO₄ solution (0.01 M, 0.416 mL, freshly prepared) was added, and the reaction mixture was shaken for another 2 hours. The crude mixture was diluted 1:10 in PBS pH 7.4, and purified using MabSelect Sure Resin (GE Healthcare, Chicago, Ill., USA), followed by size exclusion chromatography. Purified material was quantified using a Nanodrop™ UV visible spectrophotometer (using a calculated extinction coefficient based on the amino acid sequence of h-IgG1), and concentrated to approximately 10 mg/mL using a centrifugal concentrator (10,000 MWCO). Purified molecules were analyzed using 4-12% Bis Tris SDS PAGE gels by loading 1-2 μg of each molecule into the gel, and staining using instant Blue staining. Each gel included a molecular weight ladder with the indicated molecular weight standards (FIG. 29). “NR” indicates non-reduced SDS-PAGE gel condition. Maldi TOF analysis of the purified final product gave an average mass of 71189. Da (DAR=4.2). Yield 24.5 mg, 99% yield.

### Example 46—Conjugate 25 (Conjugate of h-IgG1(c-myc) Fc-2 (Int-22))

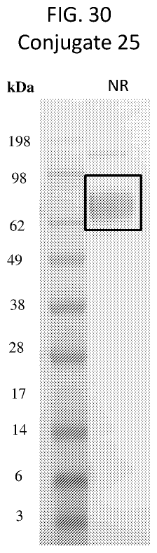

A solution of hIgG1(His)₆Fc (SEQ ID NO: 17) (20.0 mL, 3.62 μmol, 10.5 mg/mL, MW=57810 Da) in pH 7.4 PBS×1 was treated with azido-peg4-NHS ester (98%, 6.66 mg, 16.8 μmol, 10 eq), then agitated gently overnight at RT. Unreacted azido-peg4-NHS ester was removed by buffer exchange with a 10,000 MWCO centrifugal filter (6×15 mL pH 7.4 PBS×1). The final volume of the azide functionalized Fc was 9.00 mL with concentration at 12.4 mg/mL, MW=59641. To this Fc-azido PBS×1 buffer solution (0.0248 g, 0.416 μmol, 2.00 mL were added alkyne derivatized small molecule (TFA salt, 67.1 mg, 16.6 μmol, Int-22) and freshly prepared pH 7.4 PBS×1 solutions of CuSO₄ (0.832 mL of 10.0 mM, 20 eq), tris(3-hydroxypropyltriazolylmethyl)-amine (THPTA, 0.832 mL of 10.0 mM, 20 eq), ascorbic acid (0.832 mL of 10.0 mM, 20 eq). The resulting solution was agitated gently for 4 hours at room temperature. Another portion of freshly prepared pH 7.4 PBS×1 solutions of CuSO₄ (0.420 mL, of 10.0 mM, 10 eq) was added and the reaction mixture was gently agitated for overnight (˜15 hours) at room temperature, then the final portion of freshly prepared pH 7.4 PBS×1 solutions of CuSO₄ (0.420 mL, of 10.0 mM, 20 eq) was added and the reaction mixture was gently agitated for 2 hours. The crude mixture was diluted 1:10 in PBS pH 7.4, and purified using MabSelect Sure Resin (GE Healthcare, Chicago, Ill., USA), followed by size exclusion chromatography. Purified material was quantified using a Nanodrop™ UV visible spectrophotometer (using a calculated extinction coefficient based on the amino acid sequence of h-IgG1), and concentrated to approximately 10 mg/mL using a centrifugal concentrator (10,000 MWCO). Purified molecules were analyzed using 4-12% Bis Tris SDS PAGE gels by loading 1-2 μg of each molecule into the gel, and staining using instant Blue staining. Each gel included a molecular weight ladder with the indicated molecular weight standards (FIG. 30). “NR” indicates non-reduced SDS-PAGE gel condition. MALDI TOF analysis of the purified final product gave a mass of an average mass of 71842 Da (DAR=4.2). Yield 23 mg. 77% yield.

### Example 47—Conjugate 26 (Conjugate of m-IgG2a(His)₆ Fc-3 (Int-13))

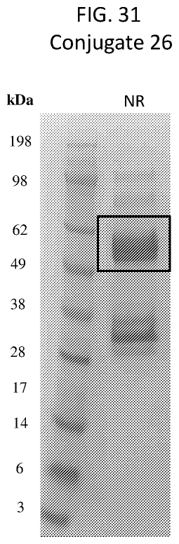

A solution of mIgG2a(His)₆ Fc (SEQ ID NO: 6) (3.1 mg in 0.6 mL pH 7.4 PBS, MW-53,782 Da), and TCEP (2.1 μL of 500 mM stock in pH 7.4 PBS, 9 eq/disulfide) were mixed at room temperature. LCMS after 1 hr showed complete reduction. The solution was diluted with 15 mL pH 7.8 PBS w/2 mM EDTA and ultrafiltered (10,000 MWCO) to a volume of 1 mL, two times. Int-10 (described in Example 12) was added as powder (1.16 mg, 2 eq/hinge disulfide). LCMS after 1 h at room temperature showed all of the reduced Fc was consumed. The crude mixture was diluted 1:10 in PBS pH 7.4, and purified using MabSelect Sure Resin (GE Healthcare, Chicago, Ill., USA), followed by size exclusion chromatography. Purified material was quantified using a Nanodrop™ UV visible spectrophotometer (using a calculated extinction coefficient based on the amino acid sequence of m-IgG2a(His)₆), and concentrated to approximately 10 mg/mL using a centrifugal concentrator (10,000 MWCO). Purified molecules were analyzed using 4-12% Bis Tris SDS PAGE gels by loading 1-2 μg of each molecule into the gel, and staining using instant Blue staining. Each gel included a molecular weight ladder with the indicated molecular weight standards (FIG. 31). Yield 2.9 mg, 82% after purification. Product peak found by LCMS 60,764 Da (DAR=2/Fc dimer).

### Example 48—Minimum Inhibitory Concentration (MIC) Assays

Generation of E. coli BW25113+pUC18-mcr-1.

An mcr-1 expression plasmid was constructed (GenScript; Piscataway, N.J.) using the pUC18 high copy number plasmid (Yanisch-Perron, et al., 1985). A 1649-bp fragment was synthesized containing, in order from 5′ to 3′: EcoR1 restriction site (5′-GAATTC-3), a ribosomal binding site (5′-AGGAGG-3′), a 5-bp native start codon upstream sequence (5′-TTCTC-3′) from the mcr-1-bearing plasmid pHNSHP45 (Liu, Y Y, et al., 2015; GenBank Accession #KP347127), the 1626-bp mcr-1 open reading frame with stop codon (“orf00073” of GenBank Accession #KP347127), and finally an Xba1 restriction site sequence (5′-TCTAGA-3′). Restriction digestion and subsequent ligation into the multiple cloning site of the pUC18 backbone resulted in directional integration of the mcr-1 gene. The pUC18-mcr-1 plasmid was transformed into chemically-competent E. coli BW25113 (Coli Genetic Stock Center #7636) as described previously (Chung, et al., PNAS. 1989), recovered for 1 h shaking at 37° C. in Super Optimal broth with Catabolite repression (SOC) media and then aliquots were plated on Luria-Bertani (LB) media containing 100 μg/mL of carbenicillin. Putative transformant colonies were then verified in MIC assays.

### Strains and Culture Conditions.

Bacterial strains were stored as glycerol stocks at −80° C. prior to culturing on cation-adjusted Mueller-Hinton agar (MHA; BD cat. no. 211438) at 37° C. Antibacterial activity was assessed against a panel consisting of Escherichia coli, Acinetobacter baumannii, Pseudomonas aeruginosa, and Klebsiella pneumoniae strains (see Table 2).

TABLE 2
Strains used in MiC assays

| Abbreviated | | | Alternative | | |
|---|---|---|---|---|---|
| name | Species | Strain ID | ID | Source | Notes |
| Ec BW | Escherichia coli | BW25113 | 7636 | CGSC | |
| Ec BW + FBS | Escherichia coii | BW25113 | 7636 | CGSC | MIC run in the |
| | | | | | presence of 50% |
| | | | | | heat-inactivated FBS |
| Ec BW | Escherichia coii | BW25113 + | 7636 | In- | COL-R; contaions |
| (mor-1) | | pUC18- | | house | mcr-1 expression |
| | | mcr-1 | | | plasmid, pUC18-mcr-1 |
| Ec BW4X-12 | Escherichia coli | BW25113 | 7636 | In-house | COL-R; spontaneous |
| (COL-R) | | 4X-12 | | | mutant (PmrB- |
| | | | | | Leu10Pro) |
| Ec 25922 | Escherichia coli | ATCC | | ATCC | |
| | | 25922 | | | |
| Ec 25922 + FBS | Escherichia coli | ATCC | | ATCC | MIC run in the |
| | | 25922 | | | presence of 50% |
| | | | | | heat-inactivated FBS |
| Ec 25922 | Escherichia coii | ATCC | | In- | COL-R; spontaneous |
| 64X-1 | | 25922 | | house | mutant (PmrB- |
| (COL-R) | | 64X-1 | | | Ala159Val) |
| Ec AR0349 | Escherichia coli | AR0349 | | CDC | COL-R; mcr-1; MDR |
| (mcr-1) | | | | | |
| Ec 2469 | Escherichia coli | ATCC | 1001728 | ATCC | MDR |
| | | BAA-2469 | | | |
| Ab 19606 | Acinetobacter | ATCC | | ATCC | |
| | baumannii | 19606 | | | |
| Ab ABS075 | Acinetobacter | ABS075- | NR-49900 | BEI | MDR |
| | baumannii | UW | | | |
| Ab 17978 | Acinetobacter | ATCC | | ATCC | |
| | baumannii | 179778 | | | |
| Ab 8990 | Acinetobacter | MMX | | Micromyx, | COL-R |
| (COL-R) | baumannii | 8990 | | LLC | |
| Pa 27853 | Pseudomonas | ATCC | | ATCC | |
| | aeruginosa | 27853 | | | |
| Pa PAO1 | Pseudomonas | PAO1 | ATCC | ATCC | |
| | aeruginosa | | BAA-47 | | |
| Pa LES431 | Pseudomonas | LES431 | BCCM | BCCM | MDR |
| | aeruginosa | | 276224 | | |
| Kp 10031 | Klebsiella | ATCC | PCI 602 | ATCC | |
| | pneumoniae | 10031 | | | |
| Kp 43816 | Klebsiella | ATCC | | ATCC | |
| | pneumoniae | 43816 | | | |
| Kp 6951 | Klebsiella | MMX | | Micromyx, | COL-R; MDR |
| (COL-R) | pneumoniae | 6951 | | LLC | |

Abbreviations: CGSC-Coli Genetic Stock Center;
FBS-fetal bovine serum;
COL-R-colistin-resistant;
ATCC-American Type Culture Collection;
MDR-multidrug-resistant;
CDC-Centers for Disease Control and Prevention;
BEI-Biodefense and Emerging infections Research Resources Repository;
BCCM-Belgian Co-Ordinated Collections of Micro-Organisms

Minimum Inhibitory Concentration (MIC) assays.

MIC assays were performed according to CLSI broth microdilution guidelines (M07-A9, M100-S23) with the exception of using a 100 μL assay volume, using RPMI media instead of Mueller-Hinton media and preparing stock compounds at 50× final concentration. Briefly, stock solutions of all antibacterial agents were prepared fresh in appropriate solvents (i.e. PBS, pH 7.4. DMSO, DI, etc.). Stock concentrations were made at 50× the highest final assay concentration and serially diluted 2-fold, 8 or 12 times in a 96-well PCR plate (VWR cat. no. 83007-374). Bacterial cell suspensions generated from MHA plate cultures were prepared in 0.85% saline and adjusted to ˜0.1 OD₆₀₀ nm. Next, cell suspensions were diluted 1:200 in RPMI containing 5% Luria Broth (LB) to improve bacterial growth (BD Difco, cat. no. 244620) to a concentration of ˜5×10⁵ CFU (colony-forming units)/mL. Due to poor solubility in Mueller-Hinton broth media, conjugates 1-15, 18-19 and 26 were evaluated initially in RPMI 1640 medium (+) L-glutamine, (−) sodium bicarbonate (“RPMI A”; MP Biomedicals, cat. no. 1060124), adjusted to pH 7.0 with NaOH and buffered with MOPS (Sigma, cat. no. M3183-500G) as is specified per CLSI guidelines for antifungal susceptibility testing (M27-A3, M27-S4). Later, conjugates 16 and 17, and 20-25 were evaluated in RPMI 1640 medium (+) L-glutamine, (+) sodium bicarbonate, pH 7.4 (“RPMI B”; Gibco, cat. no. 11875-085) as has been described as a more physiologically-relevant media for susceptibility testing of antibacterial compounds. Dorschner R A et al., The mammalian ionic environment dictates microbial susceptibility to antimicrobial defense peptides. FASEB J. 20(1):35-42 (2017). Ninety-eight microliters of each cell suspension in MHB were added to test wells in a 96-well assay plate (Costar cat. no. 3370). A Beckman Multimek 96 liquid handling robot was used to dispense 2 μL of each 50× stock compound into the plate containing 98 μL of each strain in MHB (2% final solvent concentration). E. coli strains BW25113 and ATCC 25922 were run in the presence and absence of 50% heat-inactivated fetal bovine serum (FBS; Sigma, cat. no. F4135-100 mL). Plates were mixed by shaking then incubated at 37° C. overnight (16-20 h). MIC values were read visually at 100% growth inhibition for all conjugates and control drugs (Tables 3A and 3B).

TABLE 3A
MIC values for conjugates

| | | MIC (μg/ml) | | | | | | | | |
|---|---|---|---|---|---|---|---|---|---|---|
| | | | | | Ec | | | Ec | | |
| | | | | Ec | BW | | Ec | 25922 | Ec | |
| | | Ec | Ec BW + | BW | 4X-12 | Ec | 25922 + | COL-R | AR0349 | Ec |
| Assay media | Compound | BW | FBS | (mcr-1) | (COL-R) | 25922 | FBS | 64X-1 | (mcr-1) | 2469 |
| RPMI A | COL (colistin) | 1 | 0.125 | 8 | 32 | 4 | 0.25 | ND | ND | 4 |
| RPMI B | COL (colistin) | ND | ND | ND | ND | 0.25 | 0.125 | 0.25 | 0.5 | 0.25 |
| RPMI A | Conjugate 1 | 102.4 | 12.8 | ND | ND | ND | ND | ND | ND | ND |
| RPMI A | Conjugate 2 | >128 | >128 | ND | ND | ND | ND | ND | ND | ND |
| RPMI A | Conjugate 3 | 16 | 32 | ND | ND | 64 | 64 | ND | ND | ND |
| RPMI A | Conjugate 4 | 32 | 32 | >128 | >128 | >128 | 64 | ND | ND | >128 |
| RPMI A | Conjugate 5 | 16 | 64 | 256 | >256 | >256 | 128 | ND | ND | 256 |
| RPMI A | Conjugate 6 | 4 | 8 | 8 | 16 | 32 | 16 | ND | ND | 8 |
| RPMI A | Conjugate 7 | 16 | 32 | 64 | 128 | 64 | 64 | ND | ND | 32 |
| RPMI A | Conjugate 8 | 16 | 32 | >256 | >256 | 16 | 32 | ND | ND | 16 |
| RPMI A | Conjugate 9 | 16 | 16 | >256 | 32 | 16 | 128 | ND | ND | 16 |
| RPMI A | Conjugate 10 | 16 | 64 | 128 | 256 | 16 | 128 | ND | ND | 16 |
| RPMI A | Conjugate 11 | 16 | 64 | 128 | >256 | 32 | 256 | ND | ND | 32 |
| RPMI A | Conjugate 12 | 16 | 16 | ND | ND | 64 | 256 | ND | ND | ND |
| RPMI A | Conjugate 13 | 64 | 128 | >128 | >128 | >128 | >128 | ND | ND | 128 |
| RPMI A | Conjugate 14 | 32 | >128 | >128 | >128 | >128 | >128 | ND | ND | >128 |
| RPMI A | Conjugate 15 | >128 | >128 | >128 | >128 | >128 | >128 | ND | ND | >128 |
| RPMI B | Conjugate 16 | 8 | 8 | 16 | 16 | 16 | 32 | ND | ND | 16 |
| RPMI B | Conjugate 17 | 8 | 4 | 16 | 16 | 16 | 32 | ND | ND | 16 |
| RPMI A | Conjugate 18 | 16 | 32 | 32 | 64 | 32 | 64 | ND | ND | 16 |
| RPMI A | Conjugate 19 | 16 | 8 | 32 | 64 | 32 | 32 | ND | ND | 16 |
| RPMI B | Conjugate 20 | 8 | 8 | 16 | 16 | 16 | 16 | ND | ND | 16 |

ND-not determined;
RPMI A-RPMI 1640 (+) L-glutamine, (−) sodium bicarbonate, containing 5% LB, adjusted to pH 7.0 with NaOH, buffered with MOPS;
RPMI B-RPMI 1640 (+) L-glutamine, (+) sodium bicarbonate, pH 7.4 containg 5% LB.

TABLE 3B
MIC values for conjugates

| | | MIC (μg/ml) | | | | | | | | |
|---|---|---|---|---|---|---|---|---|---|---|
| | | | | | Ec | | | Ec | | |
| | | | | Ec | BW | | Ec | 25922 | Ec | |
| | | Ec | Ec BW + | BW | 4X-12 | Ec | 25922 + | COL-R | AR0349 | Ec |
| Assay media | Compound | BW | FBS | (mcr-1) | (COL-R) | 25922 | FBS | 64X-1 | (mcr-1) | 2469 |
| RPMI B | Conjugate 21 | 8 | 8 | 16 | >16 | 64 | 128 | ND | ND | 32 |
| RPMI B | Conjugate 22 | 8 | 2 | 8 | 8 | 8 | 4 | ND | ND | 8 |
| RPMI B | Conjugate 23 | 8 | ND | 8 | 8 | 16 | 8 | 8 | 16 | 8 |
| RPMI B | Conjugate 24 | ND | ND | ND | ND | 32 | 64 | 16 | 64 | 16 |
| RPMI B | Conjugate 25 | ND | ND | ND | ND | 16 | 16 | 16 | 16 | 16 |
| RPMI A | Conjugate 26 | 16 | 32 | >128 | >128 | >128 | 128 | ND | ND | 32 |
| RPMI B | Conjugate 27 | ND | ND | ND | ND | 256 | >256 | 256 | 256 | 256 |
| RPMI B | Conjugate 28 | ND | ND | ND | ND | 32 | 64 | 32 | 32 | 32 |
| RPMI B | Conjugate 29 | ND | ND | ND | ND | 64 | 256 | 64 | 256 | 64 |
| RPMI B | Conjugate 30 | ND | ND | ND | ND | 16 | 32 | 16 | 16 | 16 |
| RPMI B | Conjugate 31 | ND | ND | ND | ND | 16 | 32 | 16 | 3 | 16 |
| RPMI B | Conjugate 32 | ND | ND | ND | ND | 256 | 256 | 128 | 256 | 256 |
| RPMI B | Conjugate 33 | ND | ND | ND | ND | 32 | 64 | 32 | 32 | 32 |
| RPMI B | Conjugate 34 | ND | ND | ND | ND | 32 | 64 | 32 | 32 | 32 |
| RPMI B | Conjugate 35 | ND | ND | ND | ND | 16 | 32 | 16 | 16 | 16 |
| RPMI B | Conjugate 36 | ND | ND | ND | ND | 64 | 128 | 32 | 64 | 64 |
| RPMI B | Conjugate 37 | ND | ND | ND | ND | 64 | 128 | 64 | 128 | 64 |
| RPMI B | Conjugate 38 | ND | ND | ND | ND | 16 | 16 | 8 | 16 | 8 |
| RPMI B | Conjugate 39 | ND | ND | ND | ND | 32 | 256 | 128 | 256 | 32 |
| RPMI B | Conjugate 40 | ND | ND | ND | ND | 32 | 64 | 16 | 32 | 32 |
| RPMI B | Conjugate 41 | ND | ND | ND | ND | 16 | 16 | 16 | 16 | 16 |
| RPMI B | Conjugate 42 | ND | ND | ND | ND | 64 | 256 | 64 | 128 | 128 |

ND-not determined;
RPMI A-RPMI 1640 (+) L-glutamine, (−) sodium bicarbonate, containing 5% LB, adjusted to pH 7.0 with NaOH, buffered with MOPS;
RPMI B-RPMI 1640 (+) L-glutamine, (+) sodium bicarbonate, pH 7.4, containing 5% LB.

TABLE 3C
MIC values for conjugates

| | | MIC (μg/ml) | | | | | | | | |
|---|---|---|---|---|---|---|---|---|---|---|
| | | | | | Ec | | | Ec | | |
| | | | | Ec | BW | | Ec | 25922 | Ec | |
| Assay | | Ec | Ec BW + | BW | 4X-12 | Ec | 25922 + | COL-R | AR0349 | Ec |
| media | Compound | BW | FBS | (mcr-1) | (COL-R) | 25922 | FBS | 64X-1 | (mcr-1) | 2469 |
| RPMI B | Conjugate 43 | ND | ND | ND | ND | 16 | 256 | 16 | 32 | 32 |
| RPMI B | Conjugate 44 | ND | ND | ND | ND | 16 | 32 | 16 | 16 | 16 |
| RPMI B | Conjugate 45 | ND | ND | ND | ND | 64 | >128 | 64 | 128 | 64 |
| RPMI B | Conjugate 46 | ND | ND | ND | ND | 64 | >128 | 64 | 128 | 128 |
| RPMI B | Conjugate 47 | ND | ND | ND | ND | 32 | 64 | 16 | 32 | 32 |
| RPMI B | Conjugate 48 | ND | ND | ND | ND | 16 | 32 | 16 | 32 | 16 |
| RPMI B | Conjugate 49 | ND | ND | ND | ND | 16 | 16 | 32 | 32 | 16 |
| RPMI B | Conjugate 50 | ND | ND | ND | ND | 64 | 64 | 64 | 64 | 64 |
| RPMI 8 | Conjugate 51 | ND | ND | ND | ND | 16 | 32 | 16 | 16 | 16 |
| RPMI B | Conjugate 52 | ND | ND | ND | ND | 16 | 16 | 16 | 16 | 16 |
| RPMI B | Conjugate 53 | ND | ND | ND | ND | 16 | 16 | 16 | 32 | 16 |
| RPMI B | Conjugate 54 | ND | ND | ND | ND | 64 | 64 | 64 | 128 | 64 |
| RPMI B | Conjugate 55 | ND | ND | ND | ND | >128 | 128 | 128 | >128 | >128 |
| RPMI B | Conjugate 56 | ND | ND | ND | ND | 32 | 32 | 32 | 32 | 32 |
| RPMI B | Conjugate 57 | ND | ND | ND | ND | 16 | 32 | 16 | 16 | 16 |
| RPMI B | Conjugate 58 | ND | ND | ND | ND | 16 | 64 | 16 | 32 | 32 |
| RPMI B | Conjugate 59 | ND | ND | ND | ND | 16 | 32 | 16 | 16 | 16 |
| RPMI B | Conjugate 60 | ND | ND | ND | ND | 16 | 32 | 16 | 16 | 16 |
| RPMI B | Conjugate 61 | ND | ND | ND | ND | 64 | 256 | 64 | 64 | 64 |
| RPMI B | Conjugate 62 | ND | ND | ND | ND | 32 | 64 | 16 | 32 | 32 |
| RPMI B | Conjugate 63 | ND | ND | ND | ND | 256 | >256 | 128 | 256 | 256 |
| RPMI B | Conjugate 64 | ND | ND | ND | ND | 32 | 128 | 32 | 32 | 32 |

ND-not determined;
RPMI A-RPMI 1640 (+) L-glutamine, (−) sodium bicarbonate, containing 5% LB, adjusted to pH 7.0 with NaOH, buffered with MOPS;
RPMI B-RPMI 1640 (+) L-glutamine, (+) sodium bicarbonate, pH 7.4, containing 5% LB.

TABLE 3D
MIC values for conjugates

| | | MIC (μg/ml) | | | | | | | | |
|---|---|---|---|---|---|---|---|---|---|---|
| | | | | | Ec | | | Ec | | |
| | | | | Ec | BW | | Ec | 25922 | Ec | |
| Assay | | Ec | Ec BW + | BW | 4X-12 | Ec | 25922 + | COL-R | AR0349 | Ec |
| media | Compound | BW | FBS | (mcr-1) | (COL-R) | 25922 | FBS | 64X-1 | (mcr-1) | 2469 |
| RPMI B | Conjugate 65 | ND | ND | ND | ND | 32 | 64 | 16 | 32 | 32 |
| RPMI B | Conjugate 66 | ND | ND | ND | ND | 64 | 128 | 64 | 64 | 64 |
| RPMI B | Conjugate 67 | ND | ND | ND | ND | 64 | 128 | 64 | 64 | 64 |
| RPMI B | Conjugate 68 | ND | ND | ND | ND | 32 | 128 | 32 | 32 | 32 |
| RPMI B | Conjugate 69 | ND | ND | ND | ND | 32 | 128 | 32 | 64 | 32 |
| RPMI B | Conjugate 70 | ND | ND | ND | ND | 64 | 256 | 64 | 128 | 64 |
| RPMI B | Conjugate 71 | ND | ND | ND | ND | 128 | >128 | 64 | 128 | 128 |
| RPMI B | Conjugate 72 | ND | ND | ND | ND | 32 | 128 | 32 | 32 | 32 |
| RPMI B | Conjugate 73 | ND | ND | ND | ND | 32 | 128 | 32 | 32 | 32 |
| RPMI B | Conjugate 74 | ND | ND | ND | ND | 32 | 64 | 16 | 32 | 32 |
| RPMI B | Conjugate 75 | ND | ND | ND | ND | 32 | 64 | 16 | 32 | 32 |
| RPMI B | Conjugate 76 | ND | ND | ND | ND | 64 | 128 | 64 | 128 | 64 |
| RPMI B | Conjugate 77 | ND | ND | ND | ND | 32 | 32 | 32 | 32 | 32 |
| RPMI B | Conjugate 78 | ND | ND | ND | ND | 64 | 128 | 32 | 64 | 64 |
| RPMI B | Conjugate 79 | ND | ND | ND | ND | 32 | 64 | 32 | 64 | 64 |

ND-not determined;
RPMI A-RPMI 1640 (+) L-glutamine, (−) sodium bicarbonate, containing 5% LB, adjusted to pH 7.0 with NaOH, buffered with MOPS;
RPMI B-RPMI 1640 (+) L-glutamine, (+) sodium bicarbonate, pH 7.4. containing 5% LB.

TABLE 3E
MIC values for conjugates

| | | MIC (μg/ml) | | | | | | | | | |
|---|---|---|---|---|---|---|---|---|---|---|---|
| Assay | | Ab | Ab | Ab | Ab 8990 | Pa | Pa | Pa | Kp | Kp | Kp 6951 |
| media | Compound | 19606 | AB5075 | 17978 | (COL-R) | 27853 | PAO1 | LES431 | 10031 | 43816 | (COL-R) |
| RPMI A | COL (colistin) | 2 | 4 | ND | ND | 1 | 4 | ND | 1 | 2 | ND |
| RPMI B | COL (colistin) | 1 | 1 | ND | 64 | 2 | 2 | 1 | 0.5 | 0.5 | 8 |
| RPMI A | Conjugate 1 | ND | ND | ND | ND | ND | 6.4 | ND | 102.4 | ND | ND |
| RPMI A | Conjugate 2 | ND | ND | ND | ND | ND | 32 | ND | >128 | ND | ND |
| RPMI A | Conjugate 3 | ND | ND | ND | ND | ND | 32 | ND | 64 | ND | ND |
| RPMI A | Conjugate 4 | 32 | 32 | ND | ND | ND | 16 | ND | 64 | >128 | ND |
| RPMI A | Conjugate 5 | 16 | 32 | ND | ND | ND | 16 | ND | 32 | 256 | ND |
| RPMI A | Conjugate 6 | 8 | 8 | ND | ND | ND | 8 | ND | 16 | 128 | ND |
| RPMI A | Conjugate 7 | 64 | 64 | ND | ND | 32 | 32 | ND | 128 | 64 | ND |
| RPMI A | Conjugate 8 | 16 | 16 | ND | ND | 16 | 16 | ND | 32 | 32 | ND |
| RPMI A | Conjugate 9 | 16 | 16 | ND | ND | 16 | 16 | ND | 2 | 32 | ND |
| RPMI A | Conjugate 10 | 32 | 16 | ND | ND | 16 | 16 | ND | 32 | 32 | ND |
| RPMI A | Conjugate 11 | 32 | 32 | ND | ND | 16 | 16 | ND | 64 | 64 | ND |
| RPMI A | Conjugate 12 | ND | ND | ND | ND | ND | 16 | ND | 64 | ND | ND |
| RPMI A | Conjugate 13 | >128 | 128 | ND | ND | ND | 16 | ND | 64 | >128 | ND |
| RPMI A | Conjugate 14 | >128 | 128 | ND | ND | ND | 128 | ND | 32 | >128 | ND |
| RPMI A | Conjugate 15 | >128 | >128 | ND | ND | ND | 16 | ND | >128 | >128 | ND |
| RPMI B | Conjugate 16 | 8 | 16 | 16 | ND | >128 | >128 | ND | 128 | >128 | ND |
| RPMI B | Conjugate 17 | 8 | 16 | 16 | ND | >128 | >128 | ND | 128 | >128 | ND |
| RPMI A | Conjugate 18 | 32 | 16 | 16 | ND | 16 | 16 | ND | 32 | 64 | ND |
| RPMI A | Conjugate 19 | 16 | 16 | 16 | ND | ND | 32 | 16 | 32 | 32 | ND |
| RPMI B | Conjugate 20 | 8 | 16 | 8 | ND | 128 | >128 | ND | 32 | 32 | ND |

ND-not determined;
RPMI A-RPMI 1640 (+) L-glutamine, (−) sodium bicarbonate, containing 5% LB, adjusted to pH 7.0 with NaOH, buffered with MOPS;
RPMI B-RPMI 1640 (+) L-glutamine, (+) sodium bicarbonate, pH 7.4, containing 5% LB.

TABLE 3F
MIC values for conjugates

| | | MIC (μg/ml) | | | | | | | | | |
|---|---|---|---|---|---|---|---|---|---|---|---|
| Assay | | Ab | Ab | Ab | Ab 8990 | Pa | Pa | Pa | Kp | Kp | Kp 6951 |
| media | Compound | 19606 | AB5075 | 17978 | (COL-R) | 27853 | PAO1 | LES431 | 10031 | 43816 | (COL-R) |
| RPMI B | Conjugate 21 | 16 | 16 | 16 | ND | >128 | >128 | ND | 128 | 128 | ND |
| RPMI B | Conjugate 22 | 8 | 8 | 8 | ND | >128 | >128 | ND | 8 | 8 | ND |
| RPMI B | Conjugate 23 | 16 | 16 | ND | 64 | 64 | >64 | 32 | 16 | 16 | 16 |
| RPMI B | Conjugate 24 | 32 | 16 | ND | >256 | >256 | >256 | 64 | 128 | >256 | 128 |
| RPMI B | Conjugate 25 | 16 | 16 | ND | 128 | 128 | 256 | 32 | 32 | 32 | 32 |
| RPMI A | Conjugate 26 | 128 | 32 | 64 | ND | 16 | 64 | ND | 32 | >128 | ND |
| RPMI B | Conjugate 27 | 256 | 256 | ND | >256 | >256 | >256 | 256 | 256 | 256 | >256 |
| RPMI B | Conjugate 28 | 32 | 32 | ND | 64 | 128 | >128 | 64 | 64 | 64 | 64 |
| RPMI B | Conjugate 29 | >256 | >256 | ND | >256 | 256 | 256 | 128 | 128 | 64 | 256 |
| RPMI B | Conjugate 30 | 16 | 16 | ND | 128 | 128 | 256 | 32 | 32 | 32 | 32 |
| RPMI B | Conjugate 31 | 16 | 16 | ND | 128 | 128 | >128 | 32 | 64 | 64 | 32 |
| RPMI B | Conjugate 32 | 256 | 128 | ND | >256 | >256 | >256 | 128 | 256 | >256 | >256 |
| RPMI B | Conjugate 33 | 32 | 32 | ND | 64 | 128 | 128 | 64 | 64 | 64 | 64 |
| RPMI B | Conjugate 34 | 32 | 32 | ND | 256 | 256 | 256 | 64 | 128 | 64 | 32 |
| RPMI B | Conjugate 35 | 16 | 16 | ND | 64 | 64 | 128 | 32 | 32 | 16 | 32 |
| RPMI B | Conjugate 36 | 32 | 32 | ND | >256 | >256 | >256 | 256 | 256 | 256 | 128 |
| RPMI B | Conjugate 37 | 32 | 32 | ND | >256 | >256 | >256 | 256 | 256 | 256 | 256 |
| RPMI B | Conjugate 38 | 8 | 16 | ND | 64 | 64 | 128 | 16 | 32 | 32 | 16 |
| RPMI B | Conjugate 39 | >256 | 64 | ND | >256 | 256 | >256 | 32 | 64 | 64 | >256 |
| RPMI B | Conjugate 40 | 16 | 16 | ND | >128 | >128 | >128 | 32 | 64 | 128 | 32 |
| RPMI B | Conjugate 41 | 16 | 16 | ND | 64 | 128 | >128 | 32 | 32 | 16 | 16 |
| RPMI B | Conjugate 42 | 32 | 32 | ND | >256 | >256 | >256 | 64 | >256 | >256 | 256 |

ND-not determined;
RPMI A-RPMI 1640 (+) L-glutamine, (−) sodium bicarbonate, containing 5% LB, adjusted to pH 7.0 with NaOH, buffered with MOPS;
RPMI B-RPMI 1640 (+) L-glutamine, (+) sodium bicarbonate, pH 7.4, containing 5% LB.

TABLE 3G
MIC values for conjugates

| | | MIC (μg/ml) | | | | | | | | | |
|---|---|---|---|---|---|---|---|---|---|---|---|
| Assay | | Ab | Ab | Ab | Ab 8990 | Pa | Pa | Pa | Kp | Kp | Kp 6951 |
| media | Compound | 19606 | AB5075 | 17978 | (COL-R) | 27853 | PAO1 | LES431 | 10031 | 43816 | (COL-R) |
| RPMI B | Conjugate 43 | 16 | 32 | ND | 32 | 64 | 64 | 16 | 64 | 64 | 32 |
| RPMI B | Conjugate 44 | 16 | 16 | ND | 128 | 256 | 256 | 16 | 32 | 64 | 32 |
| RPMI B | Conjugate 45 | 32 | 32 | ND | >128 | >128 | >128 | 32 | >128 | >128 | >128 |
| RPMI B | Conjugate 46 | 128 | 64 | ND | >128 | >128 | >128 | 128 | >128 | >128 | >128 |
| RPMI B | Conjugate 47 | 16 | 16 | ND | 256 | >256 | >256 | 64 | 256 | 256 | 128 |
| RPMI B | Conjugate 48 | 16 | 16 | ND | 128 | 128 | >128 | 32 | 32 | 64 | 32 |
| RPMI B | Conjugate 49 | 16 | 32 | ND | >128 | >128 | >128 | 64 | 64 | 64 | 32 |
| RPMI B | Conjugate 50 | 32 | 32 | ND | >128 | >128 | >128 | >128 | >128 | >128 | 128 |
| RPMI B | Conjugate 51 | 16 | 16 | ND | 128 | 128 | >128 | 64 | 32 | 32 | 32 |
| RPMI B | Conjugate 52 | 32 | 16 | ND | 128 | 256 | >256 | 32 | 32 | 32 | 32 |
| RPMI B | Conjugate 53 | 16 | 16 | ND | 64 | 128 | >128 | 64 | 32 | 32 | 32 |
| RPMI B | Conjugate 54 | 32 | 32 | ND | >128 | >128 | >128 | >128 | >128 | >128 | >128 |
| RPMI B | Conjugate 55 | 128 | >128 | ND | >128 | >128 | >128 | >128 | >128 | >128 | >128 |
| RPMI B | Conjugate 56 | 64 | 32 | ND | 128 | >128 | >128 | 64 | 128 | 128 | 64 |
| RPMI B | Conjugate 57 | 16 | 16 | ND | 128 | 128 | >128 | 32 | 32 | 32 | 32 |
| RPMI B | Conjugate 58 | 64 | 16 | ND | 256 | 256 | >256 | 64 | 64 | 64 | 64 |
| RPMI B | Conjugate 59 | 16 | 16 | ND | 256 | 256 | >256 | 64 | 32 | 32 | 32 |
| RPMI B | Conjugate 60 | 16 | 16 | ND | >256 | 256 | >256 | 64 | 64 | 128 | 32 |
| RPMI B | Conjugate 61 | 64 | 64 | ND | >512 | >512 | >512 | 64 | 256 | 256 | 128 |
| RPMI B | Conjugate 62 | 32 | 16 | ND | >512 | 512 | >512 | 64 | 64 | 64 | 64 |
| RPMI B | Conjugate 63 | 32 | 64 | ND | >256 | >256 | >256 | >256 | >256 | >256 | >256 |
| RPMI B | Conjugate 64 | 64 | 32 | ND | >512 | >512 | >512 | 64 | 128 | 64 | 128 |

ND-not determined;
RPMI A-RPMI 1640 (+) L-glutamine, (−) sodium bicarbonate containing 5% LB, adjusted to pH 7.0 with NaOH, buffered with MOPS;
RPMI B-RPMI 1640 (+) L-glutamine, (+) sodium bicarbonate, pH 7.4, containing 5% LB.

TABLE 3H
MIC values for conjugates

| | | MIC (μg/ml) | | | | | | | | | |
|---|---|---|---|---|---|---|---|---|---|---|---|
| Assay | | Ab | Ab | Ab | Ab 8990 | Pa | Pa | Pa | Kp | Kp | Kp 6951 |
| media | Compound | 19606 | AB5075 | 17978 | (COL-R) | 27853 | PAO1 | LES431 | 10031 | 43816 | (COL-R) |
| RPMI B | Conjugate 65 | 16 | 16 | ND | >128 | >128 | >128 | 32 | 128 | 128 | 64 |
| RPMI B | Conjugate 66 | 32 | 32 | ND | >128 | >128 | >128 | 64 | >128 | >128 | 128 |
| RPMI B | Conjugate 67 | >128 | 128 | ND | >128 | >128 | >128 | 64 | >128 | 128 | >128 |
| RPMI B | Conjugate 68 | 32 | 32 | ND | >128 | >128 | >128 | 64 | 64 | 64 | 64 |
| RPMI B | Conjugate 69 | 32 | 32 | ND | >128 | >128 | >128 | 64 | 128 | 128 | 128 |
| RPMI B | Conjugate 70 | 256 | 256 | ND | >256 | >256 | >256 | >256 | 256 | >256 | 256 |
| RPMI B | Conjugate 71 | >128 | 128 | ND | >128 | >128 | >128 | >128 | >128 | >128 | >128 |
| RPMI B | Conjugate 72 | 32 | 32 | ND | 128 | >128 | >128 | 32 | 128 | 128 | 64 |
| RPMI B | Conjugate 73 | 32 | 32 | ND | 64 | 128 | >128 | 32 | 128 | 128 | 64 |
| RPMI B | Conjugate 74 | 32 | 32 | ND | >256 | >256 | >256 | 256 | 128 | 64 | 64 |
| RPMI B | Conjugate 75 | 32 | 32 | ND | >128 | >128 | >128 | 128 | 64 | 64 | 64 |
| RPMI B | Conjugate 76 | 64 | 64 | ND | >128 | 256 | 256 | 64 | 128 | 128 | 128 |
| RPMI B | Conjugate 77 | 32 | 16 | ND | 256 | 256 | 256 | 32 | 64 | 64 | 32 |
| RPMI B | Conjugate 78 | 32 | 32 | ND | 64 | >128 | >128 | 32 | 128 | 128 | 64 |
| RPMI B | Conjugate 79 | 32 | 32 | ND | >256 | 256 | 256 | 64 | 128 | 128 | 64 |

ND-not determined;
RPMI A-RPMI 1640 (+) L-glutamine, (−) sodium bicarbonate, containing 5% LB, adjusted to pH 7.0 with NaOH, buffered with MOPS;
RPMI B-RPMI 1640 (+) L-glutamine, (+) sodium bicarbonate, pH 7.4. containing 5% LB.

### Example 49—Antibacterial Activity of Conjugates in Human Blood

Overnight stationary-phase cultures were centrifuged at 3,500×g for 10 min at room temperature and resuspended in 1×PBS to an optical density at 600 nm (OD₆₀₀) of 0.4 (1×10⁸ colony forming units [CFU]/mL) in RPMI with L-glutamine and phenol red (Life Technologies). Bacteria were diluted 1:25 in RPMI (4×10⁶ CFU/mL) and 10 μl added to wells of a non-binding 96-well plate (Corning 3641) containing 80 ul of 50% hirudin anti-coagulated blood (complement active) or 50% heparin anti-coagulated blood (complement inactive) in RPMI. After adding 5 ul of 20× solutions of each conjugate diluted in RPMI (128-2 ug/mL final concentration), and 5 ul RPMI for a final blood concentration 40%, the plate was incubated under static conditions at 37° C. for 90 min. Minimum bactericidal concentration (MBC) was determined by 10-fold serial dilution in 1×PBS, then plating 4 ul spots onto rectangular LB agar plates and overnight incubation at 37° C. MBC was defined as the minimum conjugate concentration required for complete bacterial clearance. An untreated growth control (40% blood, 0 μg/mL conjugate) was included in all assays. Conjugate 23 MBCs are lower in complement active hirudin-treated blood compared to complement inactive heparin-treated blood, indicating that conjugate 23-mediated killing is complement dependent (Table 4).

TABLE 4
Conjugate MBCs (μg/mL) in complement active hirudin-treated human blood vs.
(complement inactive heparin-treated human blood).

| | Bacterial strain | | | | | | |
|---|---|---|---|---|---|---|---|
| | KP 6269 | AB 6338 | PA 7874 | EC AR0349 | EC 25922 | AB 5075 | PA 01 |
| Conjugate | COL^{R} | COL^{R} | COL^{R} | COL^{R} | COL^{S} | COL^{^{S}} | COL^{S} |
| 22 | 8 (>32) | 4 (32) | >32 (>32) | 4 (32) | 2 (16) | 4 (32) | >32 (>32) |
| 23 | 4 (32) | 1 (16) | 32 (>32) | 1 (8) | <0.5 (4) | 1 (8) | 32 (>32) |

Abbreviations: KP, Klebsiella pneumoniae; AB, Acinetobacter baumannii; PA, Pseudomonas aeruginosa; EC, Escherichia coli; COL, colistin; ^{R}resistant; ^{S}sensitive.

### Example 50—Binding of Conjugates 3, 5, and 7 to Bacteria Measured by Flow Cytometry

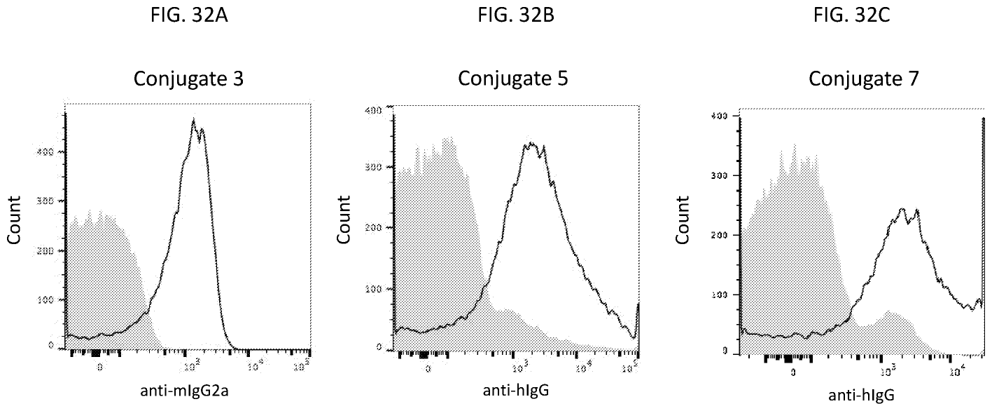

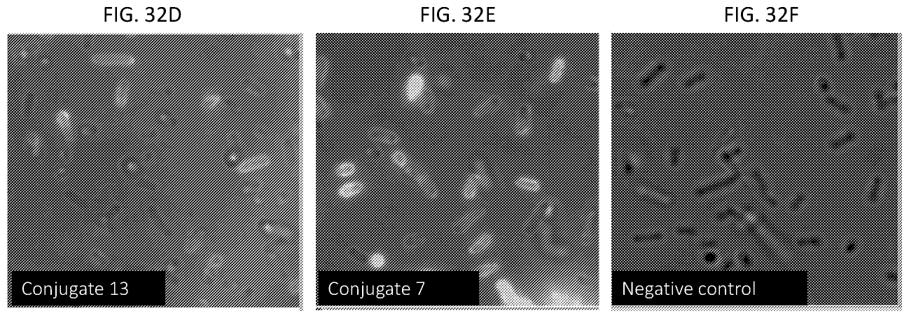

Binding of conjugates to gram negative bacteria was measured via flow cytometry. Gram negative bacteria (E. coli ATCC 25922) were grown overnight in Luria-Bertani (LB) (BD Difco™ Cat. 244620) at 37° C. while shaking. The following day, bacteria were grown to log-phase in LB until reaching an OD₆₀₀ of 0.4 (˜10⁸ CFU/mL). 1 mL of bacteria per condition were washed in DPBS pH 7.4 (Gibco 10010-023) and treated with 5 μM of Conjugate 3 (FIG. 32A), Conjugate 5 (FIG. 32B), or Conjugate 7 (FIG. 32C) (shown as open, black histograms in FIGS. 32A, 32B, and 32C) or control molecule without mlg2a-Fc (FIG. 32A) and control molecule without hIgG1-Fc (FIGS. 32B and 32C) (shown as filled, gray histograms in FIGS. 32A-32C) for 15 minutes. Bacteria were washed and molecules bound to the surface of the bacteria were detected via flow cytometry using the appropriate fluorescently-conjugated secondary antibodies. Specifically, bacteria were washed 1× in DPBS and 1× in staining buffer (20 mM HEPES (Gibco 15830-080)+1% Bovine Serum Albumin (Sigma A5611) in DPBS). Samples were blocked in staining buffer for 20 minutes at room temperature, then appropriate fluorescently conjugated secondary antibody was added (Alexa Fluor 647 conjugated, at 1:400-1:1000). Bacteria were incubated for 30 minutes in dark, washed 1× in DPBS and fixed in a 2% solution of paraformaldehyde/DPBS. 50,000 events/sample were acquired on a BD FACS Calibur and analysis was performed using FlowJo software. Viable bacteria were gated on based on FSC VS SSC properties and conjugate binding was detected as fluorescence in the PE channel. The data shown in FIGS. 32A-32C demonstrate that Conjugate 3, Conjugate 5, and Conjugate 7 are able to bind to lipopolysaccharides on the surface of E. coli ATCC 25922.

Furthermore. FIGS. 32D-32F are fluorescent images showing the binding of Conjugate 13 (FIG. 32D), Conjugate 7 (FIG. 32E), and a negative control (FIG. 32F) to E. coli, as determined by antibody binding.

### Example 51—Binding of Conjugates to FcRn

Binding of Conjugates Conjugate 15 or Conjugate 4 compared to unconjugated control Fc molecule (m-IgG2a type; SEQ ID NO: 2), to neonatal Fc receptor (FcRn) was analyzed by ELISA to determine the impact of drug conjugation on interaction with FcRn.

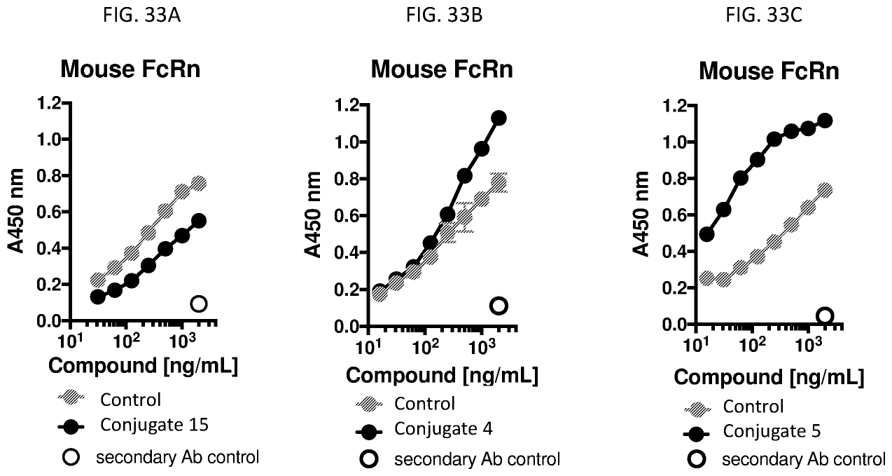

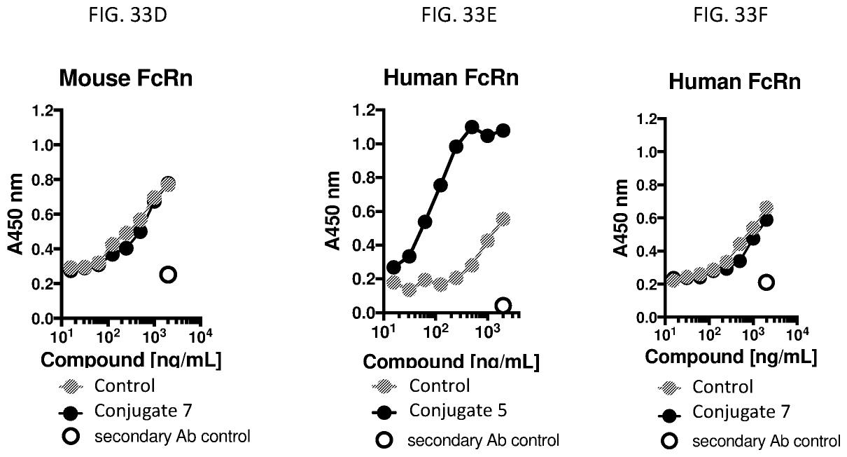

96-well ELISA plates (Fisher. Cat. No. 12-565-136) were coated with 0.25 μg/well of recombinant mouse FcRn (R&D, Cat. No. 6775-FC-050) or recombinant human FcRn (R&D, Cat. No. 8639-FC-050) in carbonate-bicarbonate buffer (Sigma, Cat. No. C34041) at pH 6.25 overnight at 37° C. A pH of 6.25 was maintained during the complete experiment. Blocking was carried out with 5% skim milk in PBS with 0.05% Tween 20. Purified conjugates were added at 20 μg/mL starting concentration and serially 2-fold diluted in blocking buffer. For detection. HRP-labeled secondary anti-mouse IgG-HRP (GE Healthcare. Cat. No. NA931V) or anti-human IgG-HRP (GE Healthcare, Cat. No. NA933V) was used to detect the bound conjugates. Absorbance was measured at 450 nm with a 96-well microplate reader and data were analyzed using GraphPad Prism software. FIGS. 33A-33F. The results demonstrate that drug conjugation in the hinge region of an Fc domain does not interfere with the conjugate's interaction with and binding to FcRn. Furthermore, hIgG1-Fc conjugates. Conjugate 5 and Conjugate 7 bind to both mouse and human FcRn.

### Example 52—Pharmacokinetics of Conjugate 23

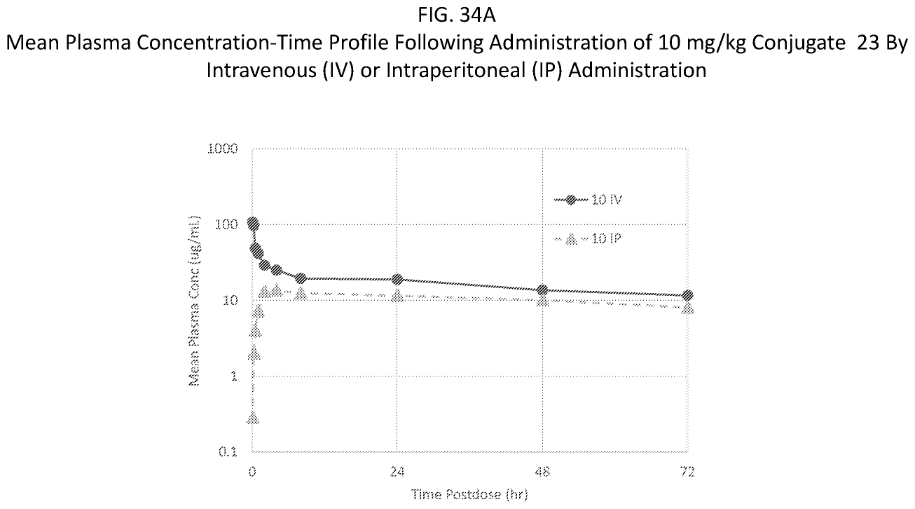

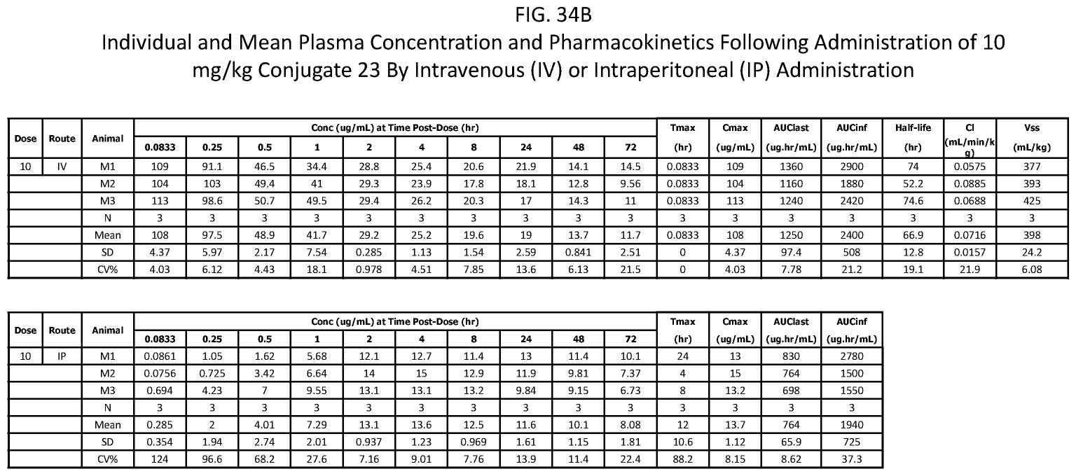

The pharmacokinetics of conjugate 23 was evaluated in male CD-1 mice (N=3 animals/group) after 10 mg/kg intravenous (IV) or intraperitoneal (IP) administration. Whole blood samples (by tail snip) were collected and the plasma harvested at 0.0833, 0.25, 0.5, 1, 2, 4, 8, 24, 48 and 72 hours post injection. Plasma concentrations of conjugate 23 were measured by sandwich ELISA using an Anti-Myc tag capture antibody (Abcam ab9132) at and an HRP-conjugated anti-hIgG-Fcg F(ab′)₂ (Jackson ImmunoResearch 709-036-098) for detection. The amount of conjugate 23 in plasma samples was quantified using a conjugate 23 standard curve. Data were analyzed using GraphPad Prism 6 and non-linear regression, Sigmoidal 4PL. Plasma pharmacokinetic parameters were then calculated by non-compartmental analysis using Phoenix WinNonlin software (FIG. 34).

### Example 53—Complement-Dependent Cytotoxicity (CDC)

To demonstrate that the conjugates can facilitate immune-mediated killing of compound bound bacteria CDC assays were performed. Gram-negative bacteria were cultured overnight in Luria-Bertani (LB) (BD Difco™ Cat. 244620) at 37° C. under constant agitation. The next day, bacteria were grown to log phase until the OD₆₀₀ reached 0.4 (˜10⁸ CFU/mL). Bacteria were washed in DPBS pH 7.4 (Gibco 10010-023) resuspended in RPMI 1640 (Gibco 11-835-055) at 10⁸ CFU/mL and 100 mL/well was added to the wells of a 96-well, TC-treated, polystyrene plate (Fisher 08-772-2C). Then 10% normal human serum ((NHS) Innovative research, IPLA-CSER), 10% heat-inactivated human serum (HIS), which is complement inactive (Innovative research, IPLA-CSER, 56 C for 30 minutes) or RPMI was added to wells. Finally, Conjugate 7 (cyclic heptapeptide hIgG1-Fc) or control (cyclic heptapeptide negative hIgG1-Fc aglycosylated hIgG1-Fc negative control molecule) were diluted to 0.2 uM final in RPMI and added to wells.

The plates were incubated at 37° C. for 90 minutes. At the end of the assay, wells were serially diluted in sterile water and remaining bacteria were cultured overnight at 37° C. on agar plates. Colonies were counted the next day and CFU/mL and % survival normalized to RPMI control was determined using the following equations: CFU/mL=(colonies*dilution factor)/volume and % inhibition normalized to RPMI=([CFU/mL of test condition]/[CFU/mL of molecule in RPMI])*100.

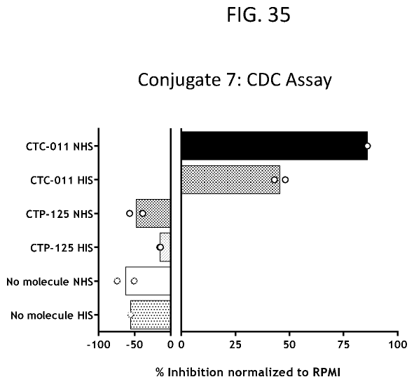

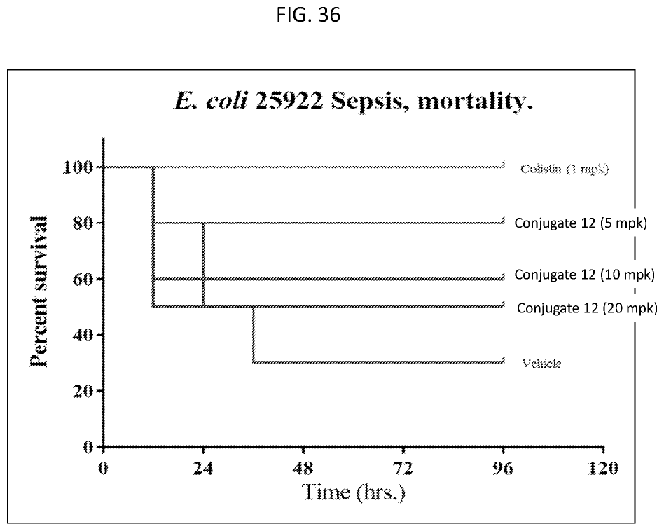

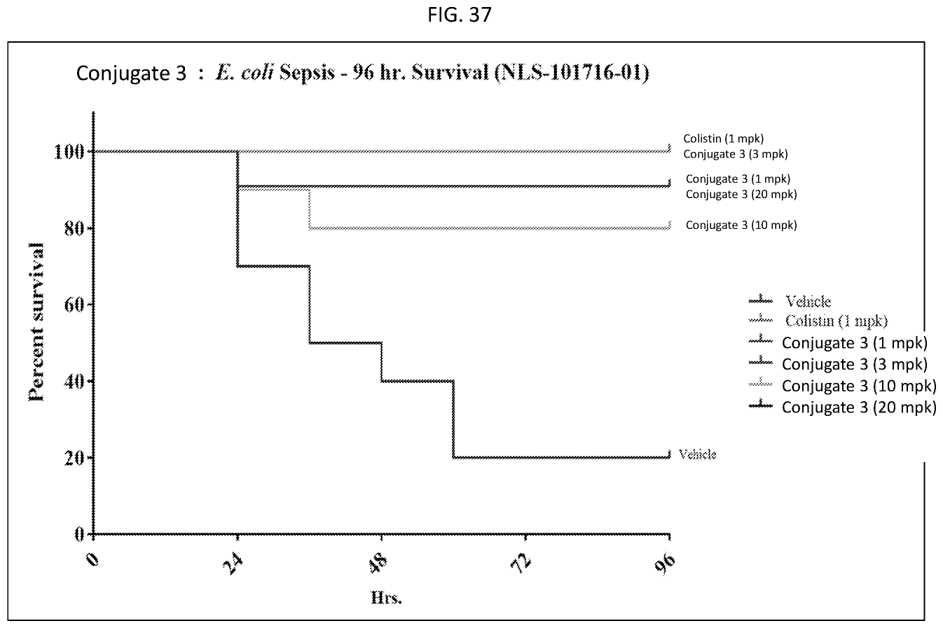

FIG. 35 shows potent antibacterial activity of Conjugate 7 in the presence of 10% complement active NHS while no antibacterial activity is seen with the control molecule. Approximately 50% of this activity is lost in the presence of 10% complement inactive HIS demonstrating the contribution of complement-dependent cytotoxicity (CDC) to the antibacterial effect. These results demonstrate Fc-mediated antibacterial activity of Conjugate 7 against E. coli ATCC 25922.

### Example 54—In Vivo Efficacy Testing of Conjugates in a Mouse Model of Septicemia

In order to better determine the potency of the conjugates, several were tested in a mouse model of peritonitis/septicemia. These studies were conducted using immunocompetent C57BL/6 mice (an inbred strain). Table 5 outlines the experimental design for a study with Conjugate 12. In this experiment the challenge pathogen was a mouse virulent isolate of E. coli (ATCC 25922) which was administered IP with 5% mucin to inhibit local macrophages (0.5 mL total volume with 0.9% saline as the vehicle; 1.4E3 CFUs of bacteria delivered). Test articles were dissolved in 0.9% saline which also served as the vehicle only control and dosed IP once as indicated in the table. The end point for the study was survival over 4-days.

TABLE 5
in Vivo Efficacy Testing of Conjugate 12 in a Mouse Model of Septicemia

| | | E. coli | | Compound | Compound | |
|---|---|---|---|---|---|---|
| | C57BL/6 | (ATCC 25922) | | (mg/kg) (IP) | (mg/kg) (IP) | End Point |
| Group | (n) | (IP) | Test Article | (T-1 hr.) | (T + H + hr.) | (Survival) |
| 1 | 10 | 1E3 | Vehicle | — | — | 4 day |
| 2 | 10 | 1E3 | Colistin | — | 1 | mortality |
| 3 | 10 | 1E3 | Conjugate 12 | 5 | — | |
| 4 | 10 | 1E3 | Conjugate 12 | 10 | — | |
| 5 | 10 | 1E3 | Conjugate 12 | 20 | — | |

The study results were plotted as a Kaplan-Meier graph (FIG. 36). In this study the death phase occurred over the first 48 hours post-bacterial challenge. All tested concentrations of Conjugate 12 (20, 10, & 5 mg/kg) showed increased survival over the vehicle only control group; as did colistin at 1 mg/kg, the positive control in the study. The difference in survival between vehicle only and the group receiving 5 mg/kg of Conjugate 12 was significantly different (Mantel-Cox test).

### Expanded Dose Titration of In Vivo Conjugate Activity in a Mouse Model of Septicemia.

In a follow up to the first efficacy study a dose titration study was run using Conjugate 3. This study was essentially performed as the previous study (Table 4) except the final challenge inoculum was 3.4E3 and all test articles/controls were dosed IV, 1 hour prior to bacterial challenge. Study results are illustrated in FIG. 37 and show substantial differences between the vehicle only control group and all concentrations of Conjugate 3 ranging from 1 to 20 mg/kg (all groups, including the colistin control are statistically different relative to the vehicle control (Mantel-Cox test). Importantly, significant efficacy was even achieved at the lowest concentration of test article run (1 mg/kg) indicating the ED₅₀ is below this level.

### Screening and Dose Titration of Conjugate 23 in a Mouse Model of Septicemia.

The activity of conjugate 23 was evaluated in vivo using an established mouse septicemia model. In this model CD-1 mice (Charles River Labs; females, 20 g) were challenged intraperitoneally (0.2 mL) with approximately 1×10⁷ CFU of E. coli 25922 grown in LB media. Prior to challenge bacteria were diluted (1:1) in fresh LB+PBS with 5% gastric hog mucin to locally inhibit macrophages.

In the first study (Table 6, study 1) conjugate 23 was administered as a single dose therapeutically 1 hr. following bacterial challenge. All animals treated with the ADC survived over the course of the study (5 days) while mice treated with vehicle only (1× PBS) reached 100% mortality. Based on the strength of these results conjugate 23 was further evaluated in this model (Table 6, study 2). In this shorter experiment an IgG1 Fc only negative control was included to exclude the possibility that the efficacy achieved in study 1 was the result of non-specific priming of the immune system by the Fc component of the ADC. As expected 4 of 5 mice treated with the negative control reached mortality by day 1 of the study. In comparison, a dose titration of conjugate 23 ranging from 6.25 to 50 mg/kg demonstrated a Stepwise (dose dependent) increase in survival with increasing conjugate 23 concentration.

Collectively. Conjugates 3, 12 and 23 showed significant efficacy in lethal models of E. coli sepsis.

TABLE 6
Screening of Conjugate 23 in a lethal mouse peritonitis/
septicemia model

| | | Route | | | % |
|---|---|---|---|---|---|
| Group | Test Article | Schedule | (n) | mg/kg | Survival |
| Study 1 | | IP @ T + 1 | | | |
| 1 | Vehicle (PBS) | | 3 | — | 0 |
| 2 | Colistin | | 3 | 0.3 | 100 |
| 3 | Conjugate 23 | | 5 | 50 | 100 |
| Study 2 | | | | | |
| 4 | Vehucle (PBS) | | 3 | — | 0 |
| 5 | Colistin | | 3 | 0.3 | 100 |
| 6 | Human IgG1 | | 5 | 50 | 20 |
| 7 | Conjugate 23 | | 5 | 50 | 80 |
| 8 | Conjugate 23 | | 5 | 25 | 60 |
| 9 | Conjugate 23 | | 5 | 12.5 | 40 |
| 10 | Conjugate 23 | | 5 | 6.25 | 0 |

### Example 55—Synergy of ADC Conjugates with Azithromycin (AZM) in an A. Baumannii Lung Infection Model

The murine pneumonia model was adapted from Lin et al., 2015 (PMID: 26288841). A. baumannii AB5075 was grown to log-phase in LB at 37° C., 200 rpm shaking. Bacteria were washed once with PBS via centrifugation at 4,000 rpm. Bacteria were adjusted to desired concentration in PBS.

Prior to infection, 8-week-old female C57BI/6J mice (Jackson Labs) were anesthetized with 100 mg/kg ketamine and 10 mg/kg xylazine. Using an otoscope, mice were infected directly into the trachea. For CFU burden studies mice were infected with 2×10⁷ CFU in 30 μL PBS. At 24 h post-infection mice were sacrificed by CO₂ and both lung lobes were harvested. Lungs were homogenized with 1 mm silica beads in 1 mL PBS using a MagNA Lyser (Roche). Homogenization was carried out at 6,000 rpm for 60 s and chilled on ice for 60 s in-between runs. Lung homogenate was diluted in PBS ranging from 10⁻¹ to 10⁻⁸, plated on LB plates and incubated at 37° C. overnight. The next day. CFU were enumerated and surviving CFU calculated relative to weight of the lung. Mice were treated intraperitoneally with colistin (10 mpk) or PBS at t=+1 h and 5 h post-infection. Conjugates or hIgG1 (50 mpk) were administered intraperitoneally at t=−12 h pre-infection and t=+1 h post-infection. Azithromycin (Fresenius-Kabi) was administered subcutaneously once at t=+1 h post-infection. Efficacy of ADC conjugates (50 mpk) was determined upon intratracheal infection of A. baumannii AB5075. For CFU burden studies, mice (n=5 per group) were infected with 3×10⁷ bacteria, sacrificed and lung CFU burden determined at 24 h time point. All ADC conjugates tested reduced CFU burden of A. baumannii AB5075—conjugate 6 by 1.12 logs (FIG. 38A), conjugate 20 by 0.91 logs (FIG. 38B) and conjugate 21 by 0.78 logs (FIG. 38C). Treatment with azithromycin (AZM) reduced CFU burden by 0.76 logs. Combination treatment of conjugates+AZM showed additive efficacy and reduced CFU burden for conjugate 6+AZM by 1.60 logs (FIG. 38A), conjugate 20+AZM by 1.81 logs (FIG. 38B) and conjugate 21+AZM by 1.50 logs (FIG. 38C).

### Example 56—Efficacy of ADC Conjugates in a Lethal A. Baumannii Lung Infection Model

The murine pneumonia model was adapted from Lin et al., 2015 (PMID: 26288841). A. baumannii AB5075 was grown to log-phase in LB at 37° C., 200 rpm shaking. Bacteria were washed once with PBS via centrifugation at 4,000 rpm. Bacteria were adjusted to desired concentration in PBS. Prior to infection, 8-week-old female C57BI/6J mice (Jackson Labs) were anesthetized with 100 mg/kg ketamine and 10 mg/kg xylazine. Using an otoscope, mice were infected directly into the trachea. Mice were treated intraperitoneally with colistin (10 mpk) or PBS at t=+1 h and 5 h post-infection. Conjugates or hIgG1 (50 mpk) were administered intraperitoneally at t=−12 h pre-infection and t=+1 h post-infection. For survival studies mice were infected with 5×10⁷ CFU in 30 μL of PBS. Mice were treated intraperitoneally with colistin (10 mpk) or PBS at t=+1 h, 5 h, 24 h, 36 h, 48 h and 60 h post-infection. Conjugates or hIgG1 (50 mpk) were administered intraperitoneally at t=−12 h pre-infection and t=+1 h post-infection. For the survival model, survival of mice (n=5 per group) was monitored for 5 days. ADC conjugate 22 showed 100% protection which is en par with colistin as positive control that was dosed at t=+1 h, t=+7 h, t=+24 h and t=+48 h. In comparison, control IgG1 resulted in only 20% protection (FIG. 39A). We found that conjugate 23 showed 100% protection and control IgG1 was not protective in this model (0%). Colistin was dosed at t=+1 h, t=+5 h, t=+24 h, t=+36 h, t=+48 h and t=+60 h and resulted in 20% survival at day 5 with mice starting to die at day 2 (FIG. 39B).

### Example 57—Measurement of Binding of ADC Conjugates to LPS Using an LPS Displacement Assay

To assess the ability of ADC conjugates to bind to LPS, we incubated purified LPS with dansyl-PMB, which becomes fluorescent upon binding to LPS. Next, we measured the change in fluorescence intensity upon addition of ADC conjugates. A decrease in fluorescence intensity by ADC conjugates reflects displaced dansyl-PMB from LPS and correlates with binding of ADC conjugates to LPS. LPS displacement was determined with fluorescent dansyl-polymyxin B (PMB). For that, 2 μL purified LPS from E. coli 0111:B4 (Sigma) at 1 mg/mL was added to 45 μL of PBS and incubated with 3 μL of 200 μM dansyl-PMB (in-house) in a black 96 well plate (Costar) for 5 min at room temperature (RT) in the dark. Test articles were added to corresponding wells in 25 μL at concentrations ranging from 20 μM to 0.2 μM. After 5 min incubation at RT in the dark, the relative fluorescence unit (RFU) is determined at excitation at 340 nm and emission at 485 nm on EnSpire plate reader (Perkin Elmer).

The % LPS displacement is calculated relative to vehicle control. % LPS displacement=(1−(RFU test article−dansyl-PMB in PBS)/(RFU no test article−dansyl-PMB in PBS))×100.

ADC conjugate 23 binds to purified LPS from E. coli O111:B4 in dose-dependency tested from 0.05-5 μM (FIG. 40A). A dependence on drug-to-antibody-ratio (DAR) on displacement was noted. Displacement was determined from conjugate 23 variants with DARs ranging from 1.5 to 4.5 (FIG. 40B) at 1 μM and (FIG. 40C) 5 μM with displaced LPS at DAR 4.5 of over 90%.

### Example 58—Measurement of Binding of ADC Conjugates to LPS Using a Macrophage Activation Assay

To address the question if binding to LPS by ADC as described in FIGS. 40A-C is sufficient to prevent down-stream LPS-induced activation of immune cells, we used a functional macrophage activation assay in which RAW macrophages produce nitrite oxide (NO) in response to LPS exposure. RAW 264.7 (ATCC TIB-71) murine macrophage cells were seeded at 2×10⁵ cells in DMEM+10% FBS per well in a 96-well plate one day prior to experiment and incubated at at 37° C./5% CO₂. The next day, the media was aspirate and cells were washed once with PBS. Test articles were prepared in RPMI-1640 without phenol-red+5% FBS and added in 100 μL to cells. After 15 min, 100 μL of 10 μg/mL LPS from E. coli O111:B4 was added to each well and incubated for 24 h at 37° C./5% CO₂. 24 h post-treatment, 150 μL of supernatant was transferred to a new 96-well plate and nitrite oxide processed according to manufacture's instructions of the Griess reagent system kit (Promega).

The % inhibition is calculated relative to PBS as vehicle control. % Inhibition=(1−(NO produced in μM test article)/(NO produced in μM vehicle control))×100. We tested the impact of ADC conjugate 23 to macrophages that were stimulated with 10 μg/mL LPS from E. coli O111:B4 to maximally induce NO production. We found that ADC conjugate 23 prevented LPS-induced macrophage activation in dose-dependency from 0.05-5 μM (FIG. 41A). Furthermore, we observed a DAR-dependent reduction in NO production from DAR 1.5 to 4.5 (FIG. 41B) at 1 μM and (FIG. 41C) 5 μM.

### Example 59—Synthesis of Int-24

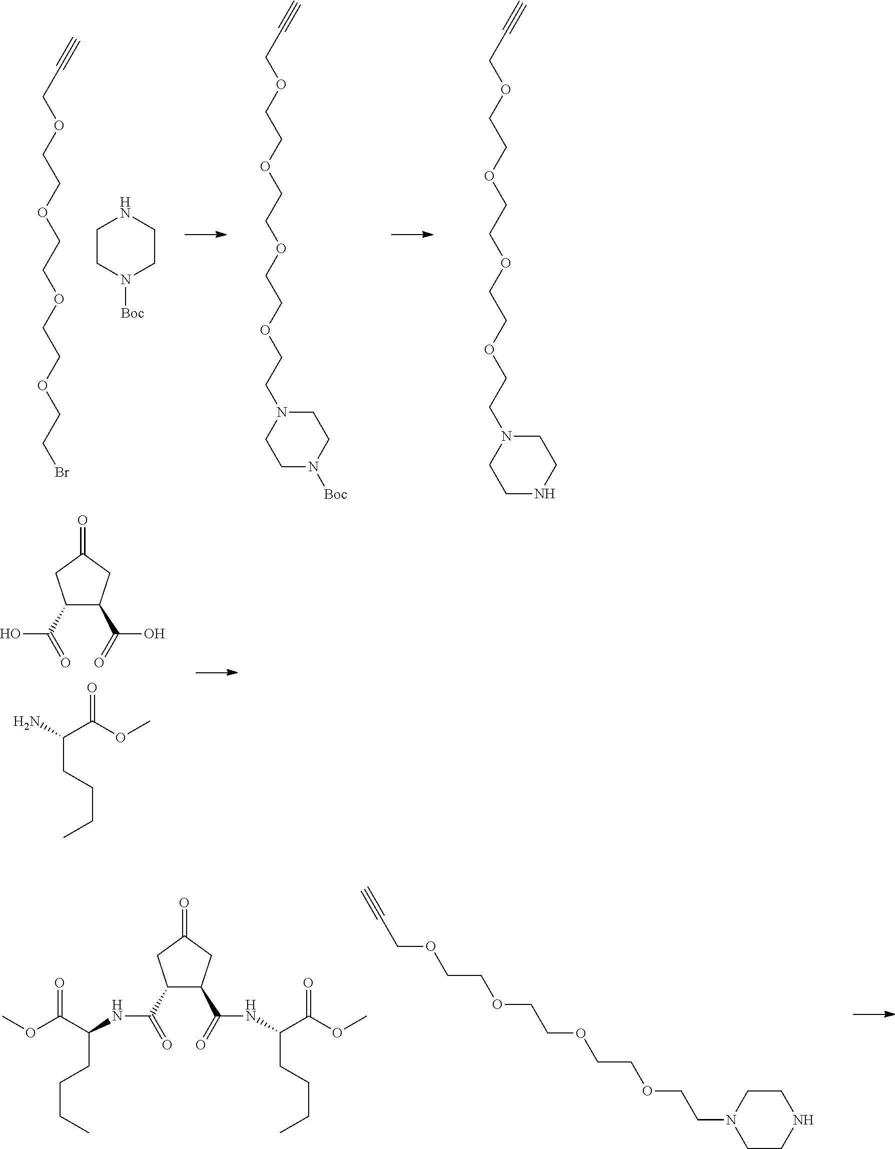

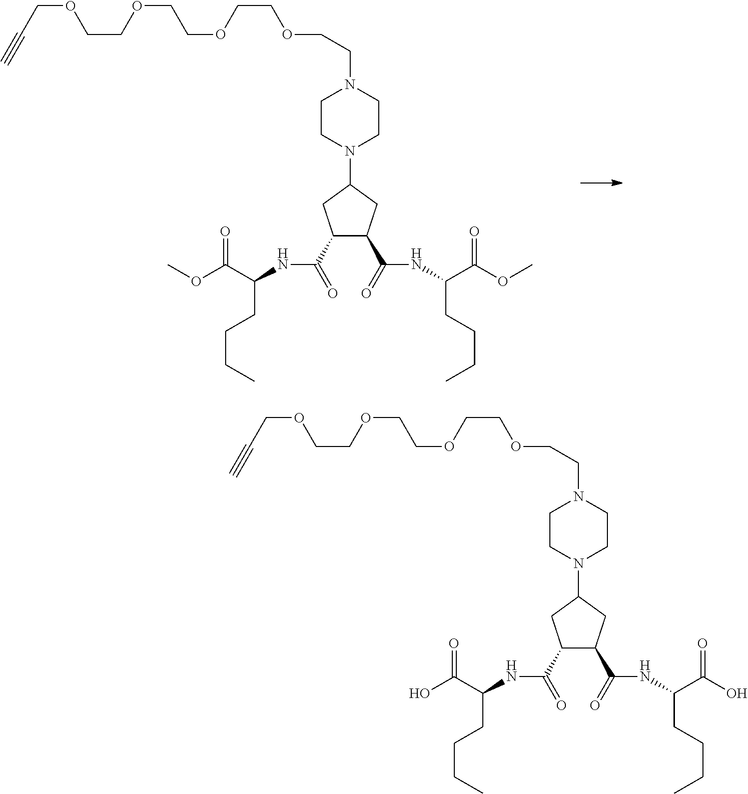

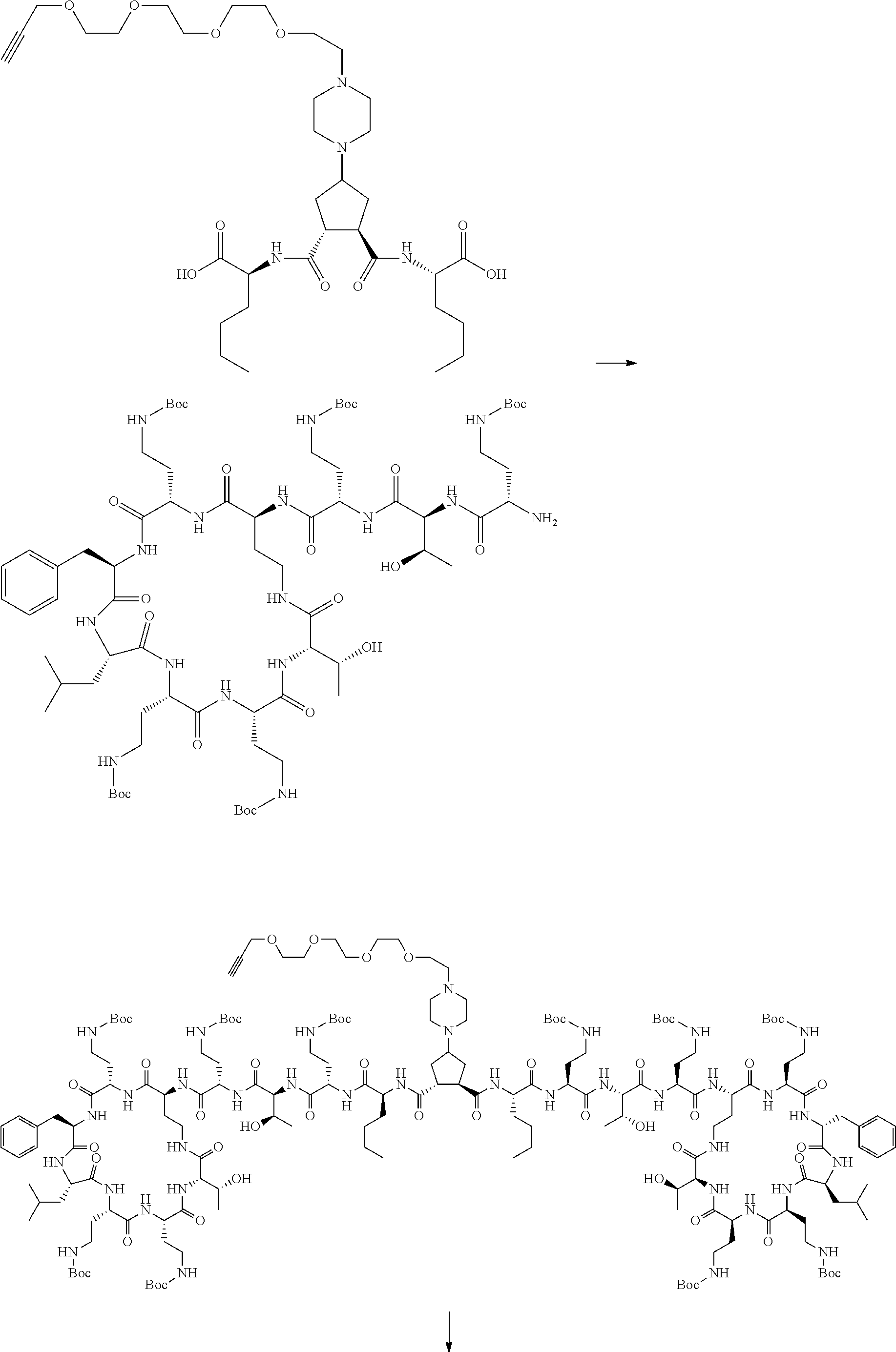

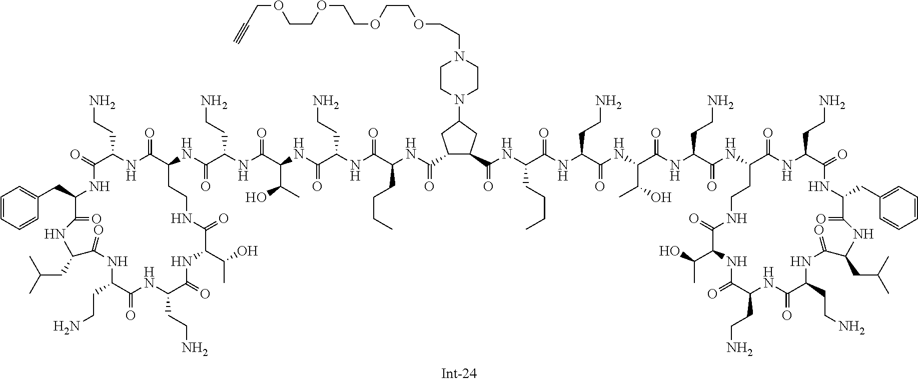 [SMILES: C#CCOCCOCCOCCOCCN1CCN(C2C[C@@H](C(=O)N[C@@H](CCCC)C(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)[C@H](C(=O)N[C@@H](CCCC)C(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)C2)CC1]

### Step a

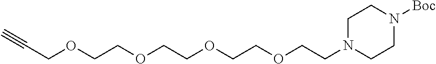 [SMILES: C#CCOCCOCCOCCOCCN1CCN(C(=O)OC(C)(C)C)CC1]

A solution of Boc-piperazine (0.316 g, 1.70 mmol), propargyl-PEG-4-bromide (0.500 g, 1.70 mmol), and potassium carbonate (0.352 g, 2.55 mmol) dissolved in acetonitrile (8 mL) was heated in a 65° C. oil bath for 15 h. The reaction was concentrated and purified by RPLC using an Isco CombiFlash liquid chromatograph eluted with 10% to 100% acetonitrile and water, using 0.1% TFA as the modifier. Yield of mono-TFA salt 0.837 g, 96% yield. Ion(s) found by LCMS: (M+H)⁺=401.4

### Step b

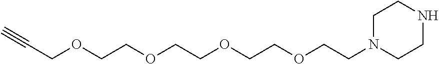 [SMILES: C#CCOCCOCCOCCOCCN1CCNCC1]

A solution of Boc-piperazine-PEG-4-alkyne (0.837 g, 1.63 mmol), and TFA (5 mL) was stirred for 5 minutes, concentrated under vacuum, and purified by RPLC using an Isco CombiFlash liquid chromatograph eluted with 10% to 100% acetonitrile and water, using 0.1% TFA as the modifier. Yield of bis-TFA salt 0.783 g, 91% yield. Ion(s) found by LCMS: (M+H)⁺=301.4

### Step c

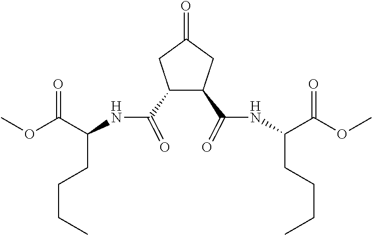

A solution of racemic trans-cyclopentanone-3, 4-dicarboxylic acid (0.500 g, 2.90 mmol), L-norleucine methylester HCl (1.16 g, 6.39 mmol), DIEA (3.03 mL, 17.4 mmol), and HATU (2.43 g, 6.39 mmol) in DMF (5 mL) was stirred at room temperature for 1 hr. The diastereomeric products were separated by RPLC using an Isco CombiFlash liquid chromatograph eluted with 10% to 100% acetonitrile and water, using 0.1% TFA as the modifier. The diastereomer with the shorter retention time was collected and taken on in the sequence. Yield of desired diastereomer 0.419 g, 34% yield. Ion(s) found by LCMS: did not ionize.

### Step d

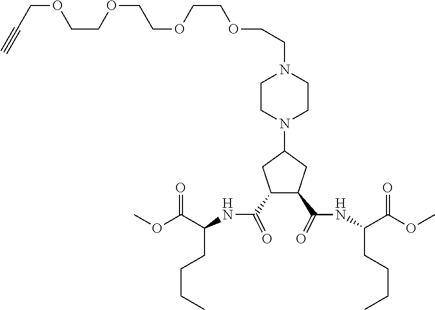 [SMILES: C#CCOCCOCCOCCOCCN1CCN(C2C[C@@H](C(=O)N[C@@H](CCCC)C(=O)OC)[C@H](C(=O)N[C@@H](CCCC)C(=O)OC)C2)CC1]

A solution of product from the previous step (0.280 g, 0.657 mmol), piperazine-PEG4-alkyne (0.416 g, 0.788 mmol), acetic acid (75 μL, 1.313 mmol), and dichloromethane (2 mL) was stirred for 30 min at room temperature, then treated with sodium triacetoxyborohydride (0.278 g, 1.31 mmol) in two equal portions 60 minutes apart. The reaction was stirred overnight, then purified by RPLC using an Isco CombiFlash liquid chromatograph eluted with 10% to 100% acetonitrile and water, using 0.1% TFA as the modifier. Yield of product was 0.275 g, 45% yield. Ion(s) found by LCMS: (M+H)⁺=711.4

### Step e

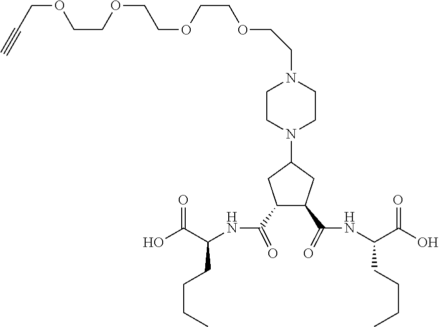 [SMILES: C#CCOCCOCCOCCOCCN1CCN(C2C[C@@H](C(=O)N[C@@H](CCCC)C(=O)O)[C@H](C(=O)N[C@@H](CCCC)C(=O)O)C2)CC1]

A solution of product from the previous step (0.275 g, 0.293 mmol) dissolved in THF (4 mL), was treated with a solution of lithium hydroxide (0.042 g, 1.76 mmol) dissolved in water (2 mL). LCMS after 30 min showed complete hydrolysis. The reaction was made slightly acidic with concentrated HCl, then purified by RPLC using an Isco CombiFlash liquid chromatograph eluted with 10% to 100% acetonitrile and water, using no modifier. Yield of product was 0.170 g, 85% yield. Ion(s) found by LCMS: (M+H)⁺=683.0

### Step f

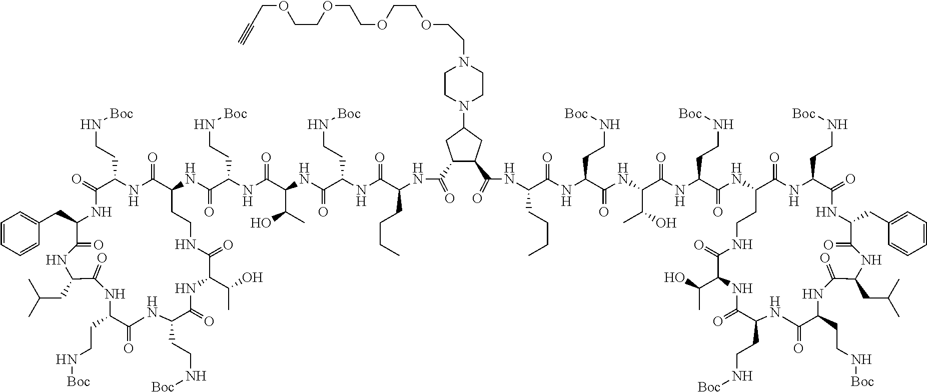

A solution of the di-acid product from the previous step (0.073 g, 0.107 mmol), INT-7a (0.351 g, 0.225 mmol, Example 8, Step a), and DIEA (0.123 mL, 0.708 mmol) dissolved in DMF (1 mL) was treated with a solution of HATU (0.090 g, 0.236 mmol), in DMF (1 mL), dropwise over 60 minutes. Product was isolated by RPLC using an Isco CombiFlash liquid chromatograph eluted with 30% to 100% methanol and water, using 0.1% TFA as the modifier. Yield of 0.350 g, 82% yield. Ion(s) found by LCMS: [M−2(Boc)/3]+H+=1191.7, [M−3(Boc)/3]+H+=1158.4, [M−3(Boc)/4]+H+=869.0, [M−4(Boc)/4]+H+=843.7.

### Step g

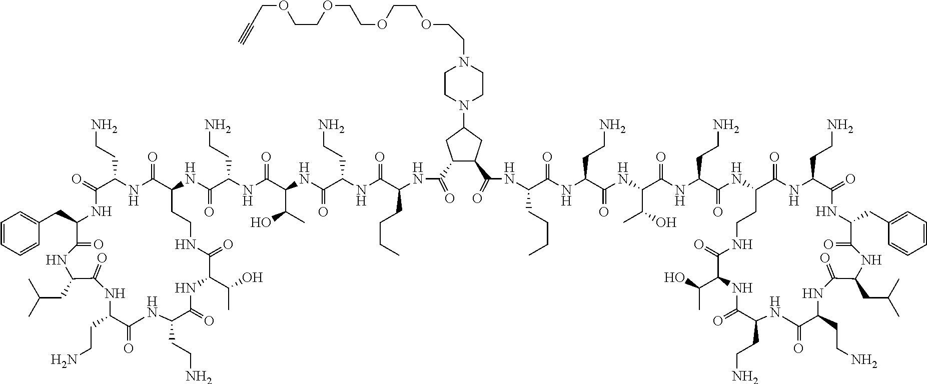 [SMILES: C#CCOCCOCCOCCOCCN1CCN(C2C[C@@H](C(=O)N[C@@H](CCCC)C(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)[C@H](C(=O)N[C@@H](CCCC)C(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)C2)CC1]

A solution of the deca-boc-protected product from the previous step (0.350 g, 0.093 mmol), was stirred in TFA (3 mL) for five minutes, and then stripped of TFA using a rotary evaporator. Product was isolated by RPLC using an Isco CombiFlash liquid chromatograph eluted with 5% to 100% methanol and water, using 0.1% TFA as the modifier. Yield of the product (Int-24) as a TFA salt was 0.340 g, 88% yield. Ion(s) found by LCMS: [M/4]+H+=924.6, [M/5]+H+=694.1, [M/6]+H+=555.3. [M/7]+H+=463.0.

### Example 60—Synthesis of Int-25

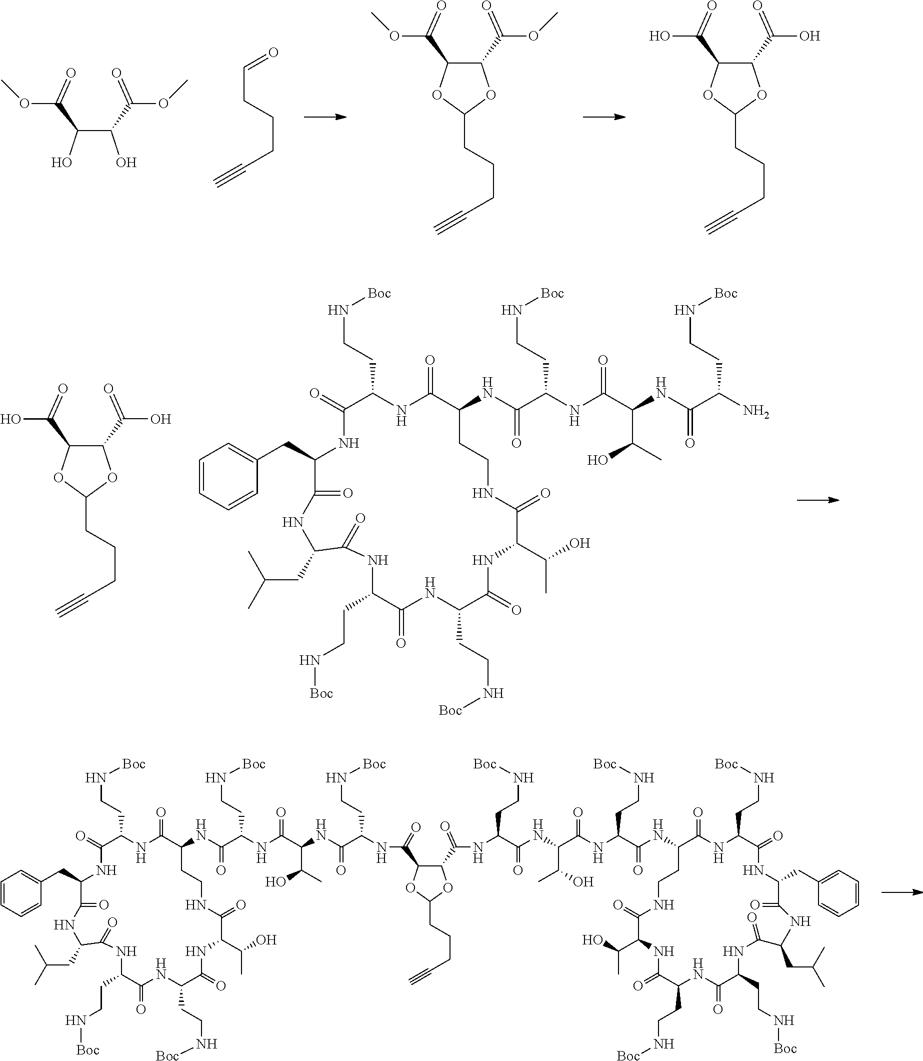

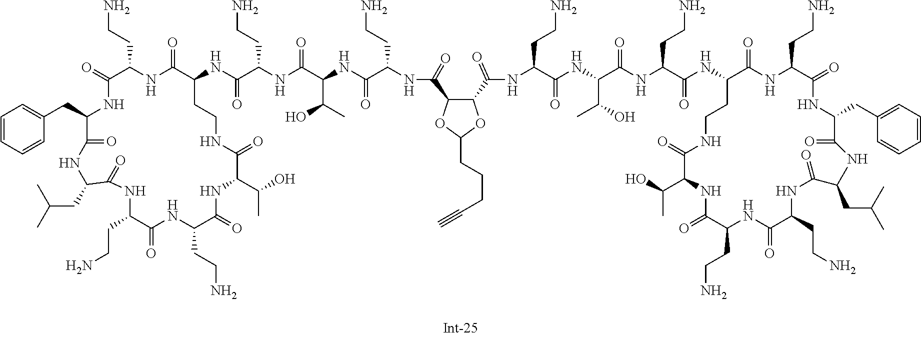 [SMILES: C#CCCCC1O[C@@H](C(=O)N[C@@H](CCN)C(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)[C@H](C(=O)N[C@@H](CCN)C(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)O1]

### Step a

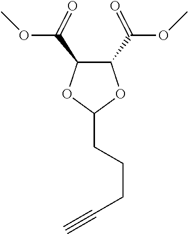

A flask charged with L-dimethyltartrate (1.25 g, 7.02 mmol), 5-pentynal (1.23 mL, 10.5 mmol), p-toluenesulfonic acid (0.120 g, 0.702 mmol), and toluene (35 mL), was equipped with a Dean-Stark trap and heated in a 155° C. oil bath. After 30 minutes, approximately 0.4 mL of water was collected. The reaction was cooled down, then stripped of toluene and purified by RPLC using an Isco CombiFlash liquid chromatograph eluted with 10% to 100% methanol and water, using no modifier. Yield of product was 0.854 g, 47% yield. Ion(s) found by LCMS: (M+H)⁺=257.2

### Step b

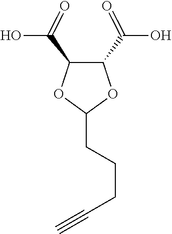 [SMILES: C#CCCCC1O[C@@H](C(=O)O)[C@H](C(=O)O)O1]

A solution of the bis-ester from the previous step (0.850 g, 3.32 mmol), dissolved in THF (6 mL), was treated with a solution of lithium hydroxide (0.238 g, 9.95 mmol) dissolved in water (3 mL). LCMS after 30 minutes indicated complete reaction. The reaction was made slightly acid with conc. HCl, stripped of THF, and purified by RPLC using an Isco CombiFlash liquid chromatograph eluted with 5% to 100% acetonitrile and water, using no modifier. Yield of product was 0.817 g, 108% yield. The product was contaminated with salt, and was used in the next step without further purification. Ion(s) found by LCMS: (M+H)⁺=229.2

### Step c

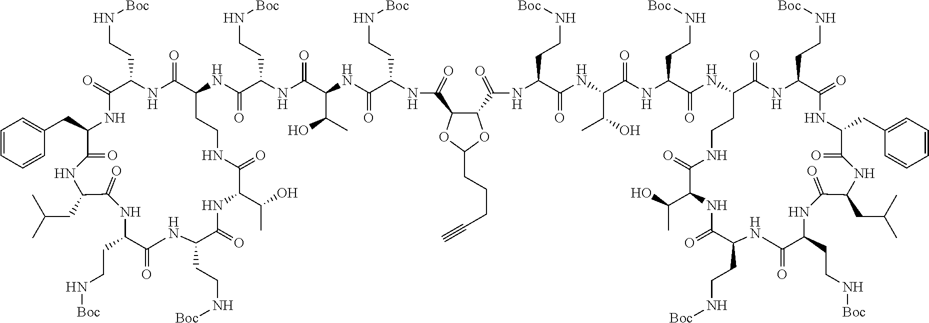

A solution of di-acid product from the previous step (0.035 g, 0.153 mmol), Int-7a (0.505 g, 0.323 mmol, Example 8, Step b), and DIEA (0.177 mL, 1.02 mmol) dissolved in DMF (2 mL) was treated with a solution of HATU (0.129 g, 0.339 mmol), in DMF (1 mL), dropwise over 60 minutes. The product was isolated by RPLC using an Isco CombiFlash liquid chromatograph eluted with 30% to 100% methanol and water, using no modifier. Yield of product was 0.361 g, 67% yield. Ion(s) found by LCMS: [M−3(Boc)/3]+H+=1007.4, [M−4(Boc)/3]+H+=973.7, [M−5(Boc)/3]+H+=940.3.

### Step d

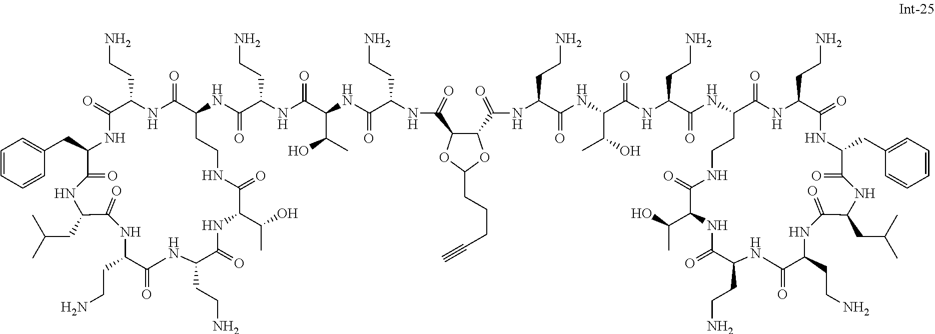 [SMILES: C#CCCCC1O[C@@H](C(=O)N[C@@H](CCN)C(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)[C@H](C(=O)N[C@@H](CCN)C(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)O1]

A solution of the deca-boc-protected product from the previous step (0.361 g, 0.109 mmol), was stirred in TFA (3 mL) for five minutes and then stripped of TFA using a rotary evaporator. Product was isolated by RPLC using an Isco CombiFlash liquid chromatograph eluted with 5% to 100% methanol and water, using 0.1% TFA as the modifier. Yield of Int-25 as a TFA salt was 0.350 g, 93% yield. Ion(s) found by LCMS: [M/3]+H+=773.3, [M/4]+H+=580.4, [M/5]+H+=464.7.

### Example 61—Synthesis of Int-26

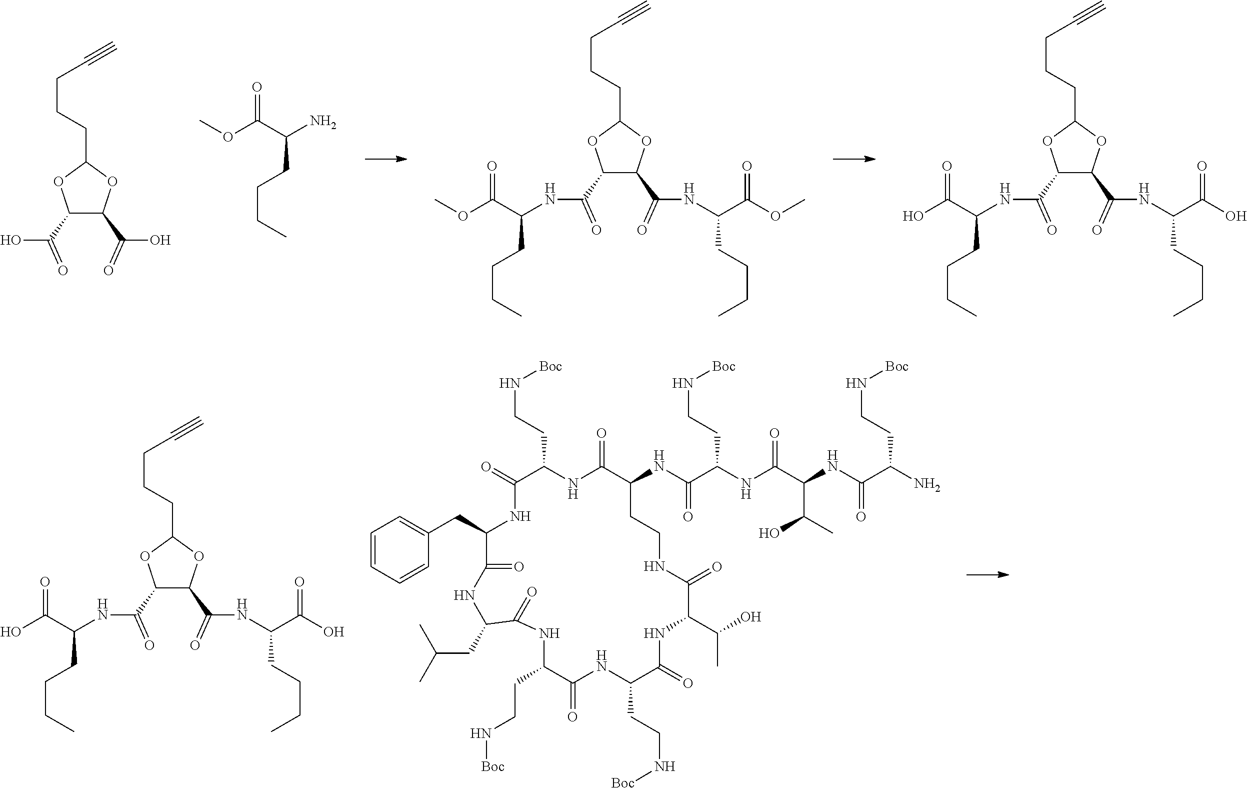

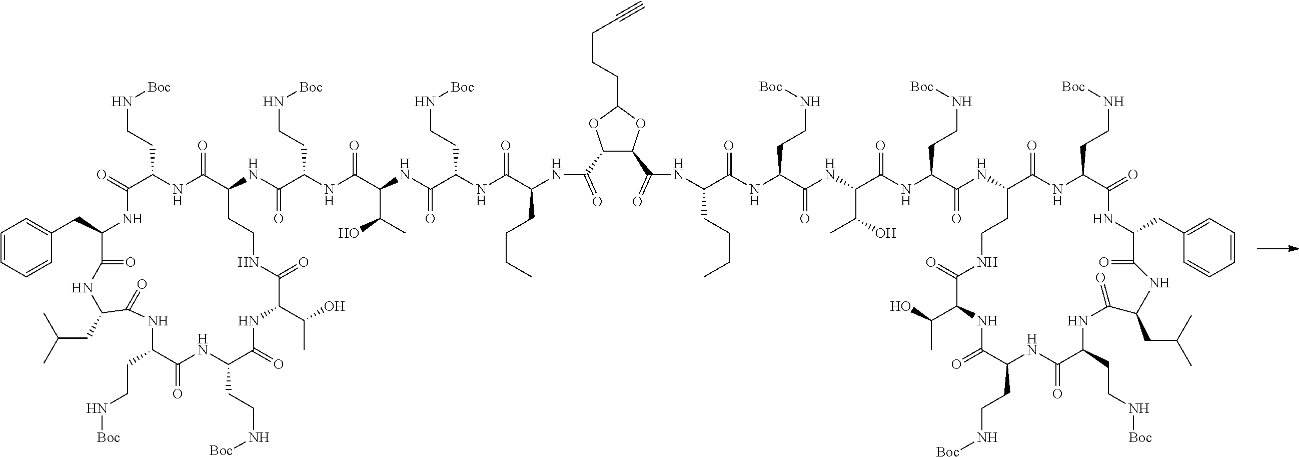

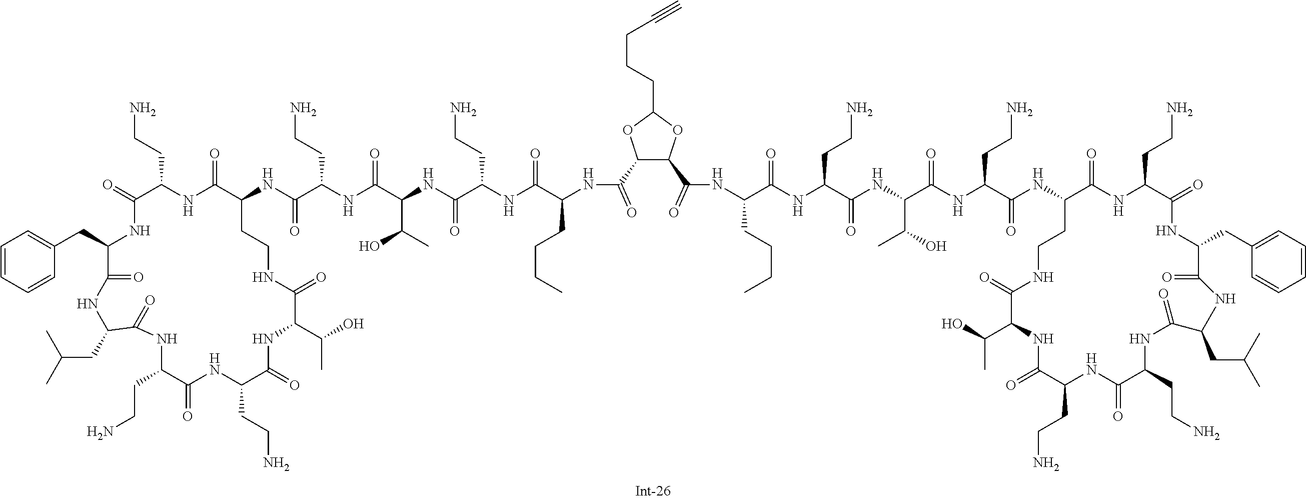 [SMILES: C#CCCCC1O[C@@H](C(=O)N[C@@H](CCCC)C(=O)N[C@@H](CCN)C(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)[C@H](C(=O)N[C@@H](CCCC)C(=O)N[C@@H](CCN)C(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)O1]

### Step a

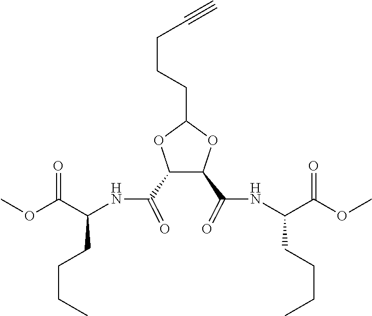

A solution of diacid (0.200 g, 0.876 mmol, Example 60, step b), L-norleucine methyl ester hydrochloride (0.255 g, 1.753 mmol), and DIEA (0.962 mL, 5.52 mmol), in DMF (4 mL) was treated with HATU (0.700 g, 1.841 mmol) in one portion, at room temperature. After stirring for 30 minutes, the reaction was purified by RPLC using an Isco CombiFlash liquid chromatograph eluted with 5% to 100% methanol and water, using no modifier. The product containing fractions were combined and concentrated to yield 0.210 g, 50%. Ion(s) found by LCMS: (M+H)⁺=483.3

### Step b

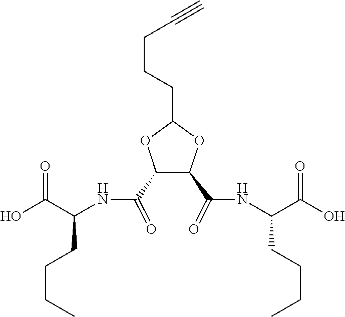 [SMILES: C#CCCCC1O[C@@H](C(=O)N[C@@H](CCCC)C(=O)O)[C@H](C(=O)N[C@@H](CCCC)C(=O)O)O1]

A solution of the diester from Step a (0.210 g, 0.435 mmol), in THF (2 mL) was treated with a solution of lithium hydroxide (0.026 g, 1.088 mmol) dissolved in water (1 mL). LCMS after 30 minutes showed complete hydrolysis. Product was purified by RPLC using an Isco CombiFlash liquid chromatograph eluted with 5% to 100% methanol and water, using no modifier. Yield of the desired diacid was 0.195 g, 98%. Ion(s) found by LCMS: (M+H)⁺=455.2

### Step c

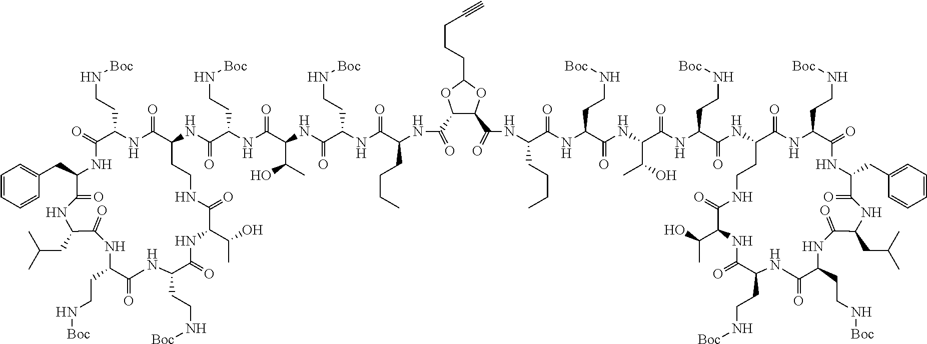

A solution of diacid product from the previous step (0.050 g, 0.110 mmol), Int-7a (0.326 g, 0.232 mmol, Example 8, Step b), and DIEA (0.121 mL, 0.695 mmol) dissolved in DMF (2 mL) was treated with a solution of HATU (0.093 g, 0.243 mmol), in DMF (1 mL), dropwise over 60 minutes. Product was isolated by RPLC using an Isco CombiFlash liquid chromatograph eluted with 30% to 100% methanol and water, using no modifier. Yield of the desired product was 0.303 g, 74% yield. Ion(s) found by LCMS: [M−2(Boc)/3]+H+=1115.5. [M−3(Boc)/3]+H+=1082.7. [M−4(Boc)/3]+H+=1049.2.

### Step d.

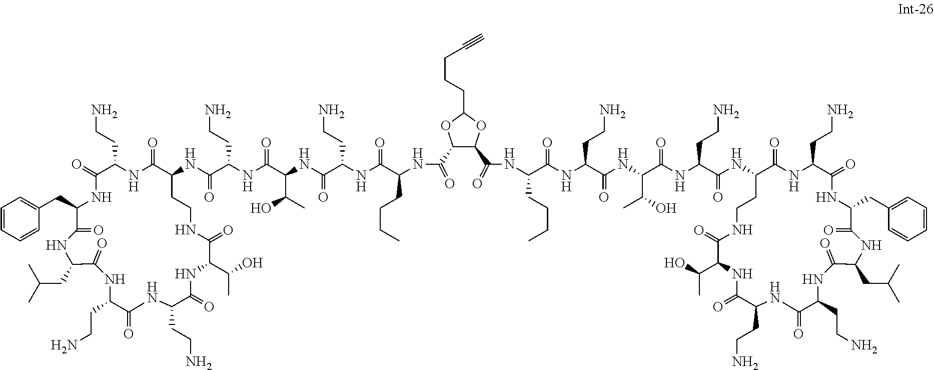 [SMILES: C#CCCCC1O[C@@H](C(=O)N[C@@H](CCCC)C(=O)N[C@@H](CCN)C(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)[C@H](C(=O)N[C@@H](CCCC)C(=O)N[C@@H](CCN)C(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)O1]

A solution of the deca-boc-protected product from the previous step (0.303 g, 0.085 mmol), was stirred in TFA (2 mL) for five minutes, and then stripped of TFA using a rotary evaporator. The product was isolated by RPLC using an Isco CombiFlash liquid chromatograph eluted with 5% to 100% methanol and water, using 0.1% TFA as the modifier. Yield of Int-26 as a TFA salt was 0.274 g, 87% yield. Ion(s) found by LCMS: [M/3]+H+=849.1, [M/4]+H+=637.2, [M/5]+H+=510.0.

### Example 62—Synthesis of Int-27

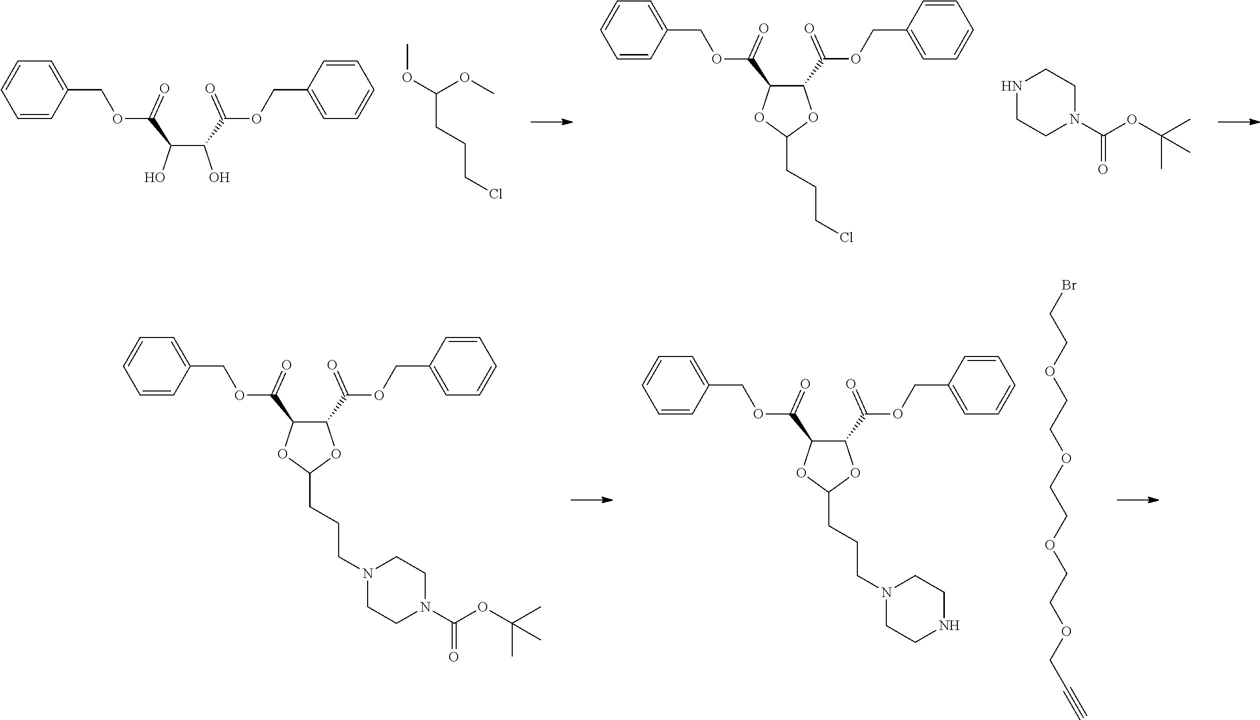

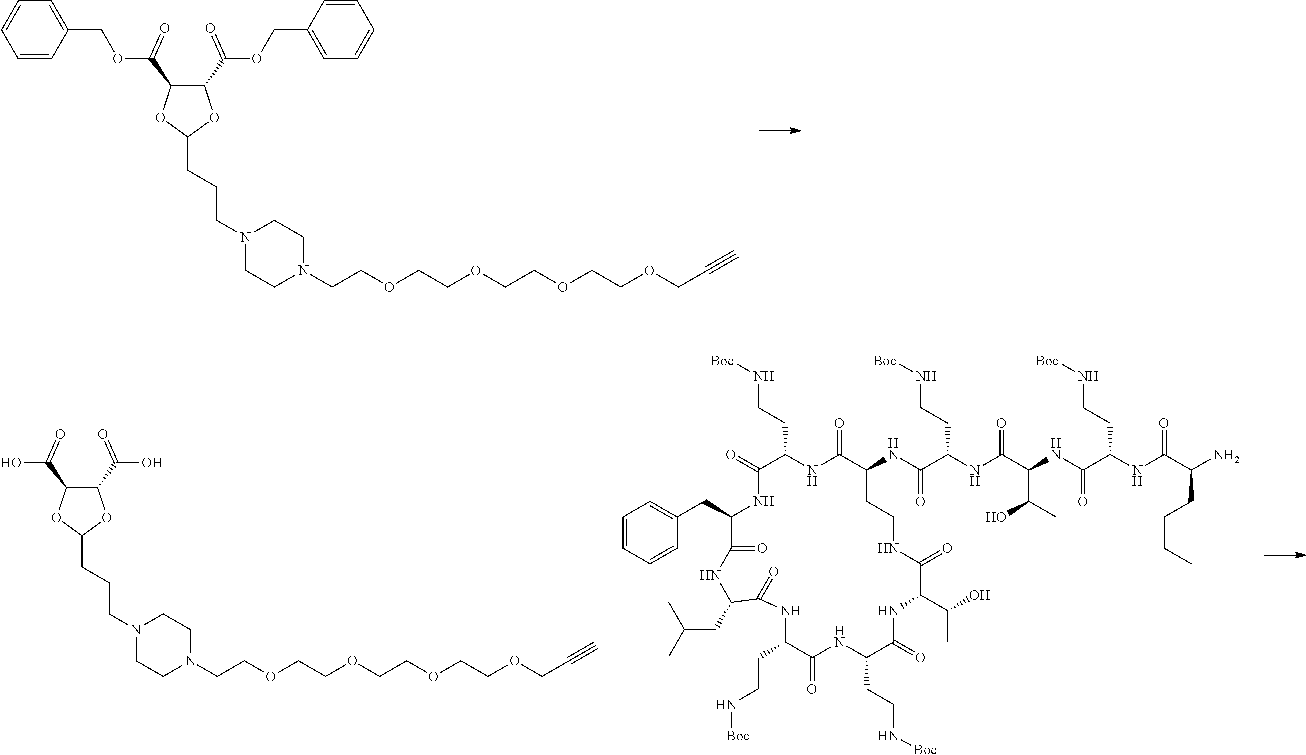 [SMILES: C#CCOCCOCCOCCOCCN1CCN(CCCC2O[C@@H](C(=O)OCc3ccccc3)[C@H](C(=O)OCc3ccccc3)O2)CC1 ; C#CCOCCOCCOCCOCCN1CCN(CCCC2O[C@@H](C(=O)O)[C@H](C(=O)O)O2)CC1 ; CCCC[C@H](N)C(=O)N[C@@H](CCNC(=O)OC(C)(C)C)C(=O)N[C@H](C(=O)N[C@@H](CCNC(=O)OC(C)(C)C)C(=O)N[C@H]1CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](Cc2ccccc2)NC(=O)[C@H](CCNC(=O)OC(C)(C)C)NC1=O)[C@@H](C)O]

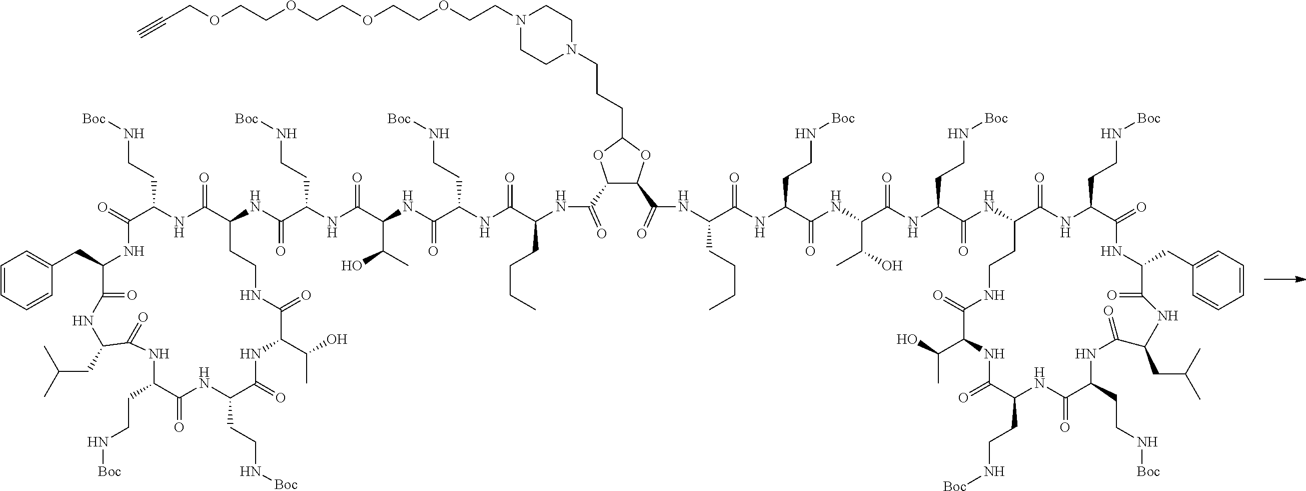

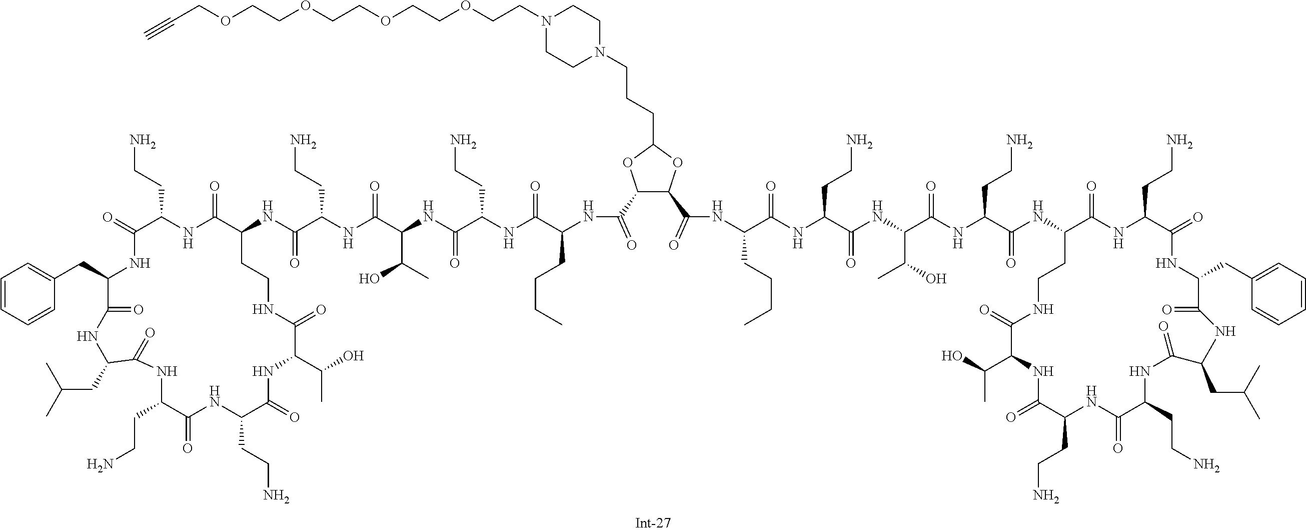 [SMILES: C#CCOCCOCCOCCOCCN1CCN(CCCC2O[C@@H](C(=O)N[C@@H](CCCC)C(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)[C@H](C(=O)N[C@@H](CCCC)C(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)O2)CC1]

### Step a

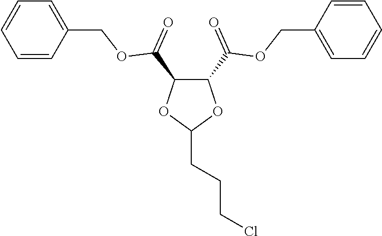 [SMILES: O=C(OCc1ccccc1)[C@@H]1OC(CCCCl)O[C@H]1C(=O)OCc1ccccc1]

Neat 4-chloro butanal dimethyl acetal (1.85 g, 12.1 mmol), L-dibenzyl tartrate (1.0 g, 3.03 mmol), and methanesulfonic acid (0.029 mL, 0.454 mmol) were heated in an 80° C. oil bath for 1 hr. The crude reaction was purified directly by RPLC using an Isco CombiFlash liquid chromatograph eluted with 30% to 100% acetonitrile and water, using no modifier. Yield 0.122 g, 9.6% yield. Ion(s) found by LCMS: (M+H)⁺=419.2

### Step b

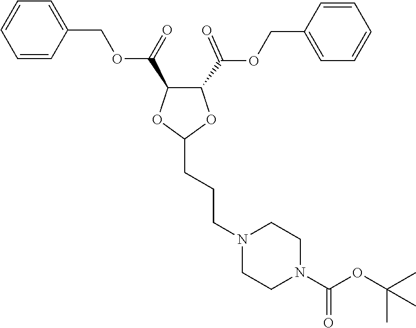 [SMILES: CC(C)(C)OC(=O)N1CCN(CCCC2O[C@@H](C(=O)OCc3ccccc3)[C@H](C(=O)OCc3ccccc3)O2)CC1]

A solution of the diester from Step a (0.244 g, 0.583 mmol), Boc-piperazine (0.434 g, 2.33 mmol), potassium iodide (0.10 g, 0.583 mmol), and sodium bicarbonate (0.098 g, 1.17 mmol) dissolved in DMF (0.5 mL) was heated in an 80° C. oil bath for 3 hours. The crude reaction was purified directly by RPLC using an Isco CombiFlash liquid chromatograph eluted with 10% to 100% acetonitrile and water, using no modifier. Yield 0.134 g, 40% yield. Ion(s) found by LCMS: (M+H)⁺=569.4

### Step c

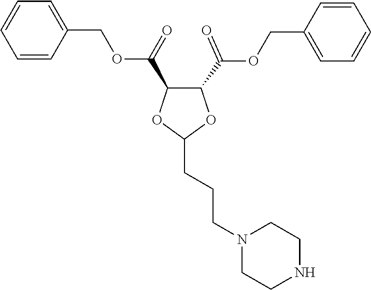 [SMILES: O=C(OCc1ccccc1)[C@@H]1OC(CCCN2CCNCC2)O[C@H]1C(=O)OCc1ccccc1]

Boc protected diester from Step b (0.215 g, 0.378 mmol) was treated with TFA (2 mL) for 5 minutes, then stripped of TFA under vacuum, and purified by directly by RPLC using an Isco CombiFlash liquid chromatograph eluted with 10% to 100% acetonitrile and water, using no modifier. Yield 0.116 g, 65% yield. Ion(s) found by LCMS: (M+H)⁺=469.2.

### Step d

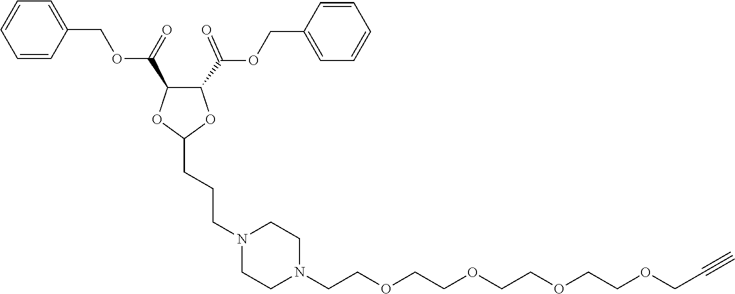 [SMILES: C#CCOCCOCCOCCOCCN1CCN(CCCC2O[C@@H](C(=O)OCc3ccccc3)[C@H](C(=O)OCc3ccccc3)O2)CC1]

A solution of product from the previous step (0.116 g, 0.248 mmol), propargyl-PEG4-bromide (0.110 g, 0.371 mmol), and sodium bicarbonate (0.062 g, 0.743 mmol) were dissolved in DMF (2 mL) and heated in an 80° C. oil bath for 4 hr. Product was isolated by RPLC using an Isco CombiFlash liquid chromatograph eluted with 10% to 100% acetonitrile and water, using no modifier. Yield 0.072 g, 43% yield. Ion(s) found by LCMS: (M+H)⁺=683.3

### Step e

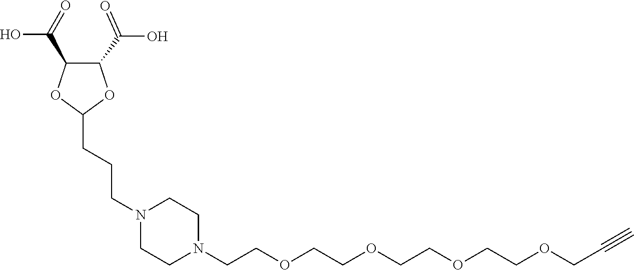 [SMILES: C#CCOCCOCCOCCOCCN1CCN(CCCC2O[C@@H](C(=O)O)[C@H](C(=O)O)O2)CC1]

A solution of the dibenzyl ester from Step d (0.072 g, 0.105 mmol), in THF (1 mL) was treated with a solution of lithium hydroxide (0.0076 g, 0.316 mmol) dissolved in water (0.5 mL). LCMS after 30 minutes showed complete hydrolysis. The reaction was made slightly acidic with conc. HCl then purified by RPLC using an Isco CombiFlash liquid chromatograph eluted with 5% to 100% methanol and water, using no modifier. Yield 0.052 g, 98%. Ion(s) found by LCMS: (M+H)⁺=503.5.

### Step f

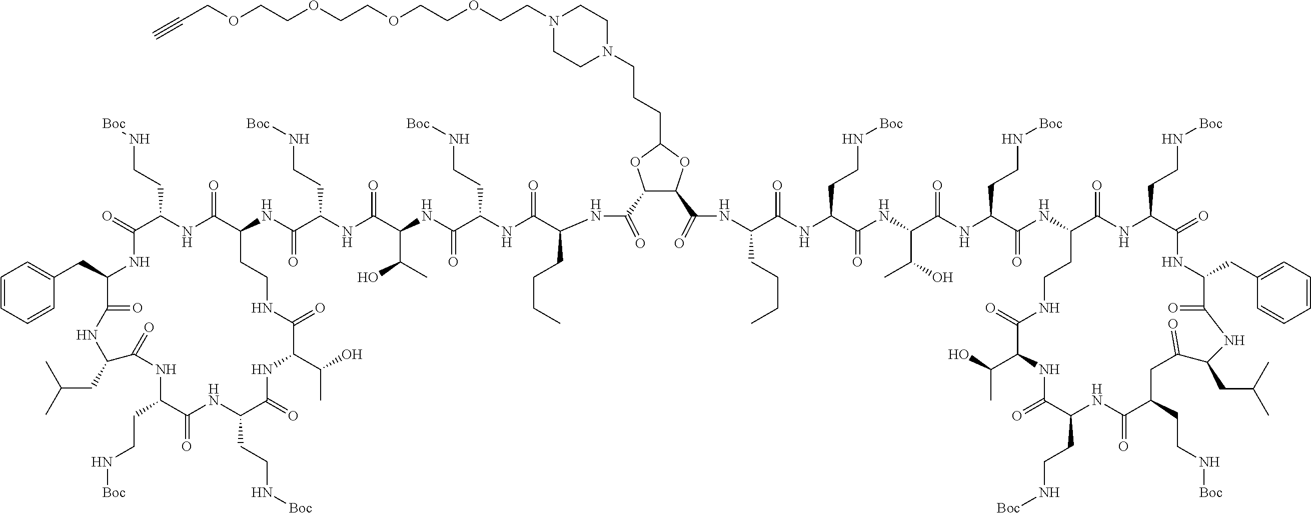

A solution of di-acid product from the previous step (0.065 g, 0.089 mmol), Int-75 (0.326 g, 0.232 mmol, Example 104, step e), and DIEA (0.155 mL, 0.890 mmol) dissolved in DMF (2 mL) was treated with a solution of HATU (0.117 g, 0.306 mmol), in DMF (1 mL), dropwise over 60 minutes. Product was isolated by RPLC using an Isco CombiFlash liquid chromatograph eluted with 30% to 100% methanol and water, using no modifier. Yield of 0.228 g, 57% yield. Ion(s) found by LCMS: [M−3(Boc)/4]+H+=880.6, [M−4(Boc)/4]+H+=855.5. [M−5(Boc)/4]+H+=830.5.

### Step g

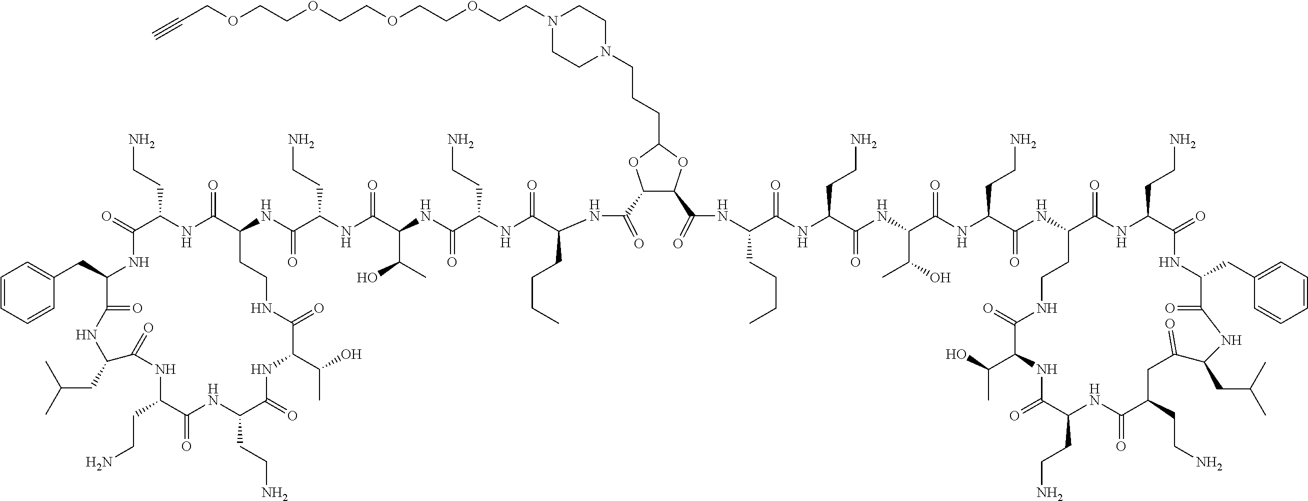 [SMILES: C#CCOCCOCCOCCOCCN1CCN(CCCC2O[C@@H](C(=O)N[C@@H](CCCC)C(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)CC(=O)[C@H](CC(C)C)NC(=O)[C@@H](Cc4ccccc4)NC(=O)[C@H](CCN)NC3=O)[C@@H](C)O)[C@H](C(=O)N[C@@H](CCCC)C(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)O2)CC1]

A solution of deca-boc-protected product from the previous step (0.228 g, 0.060 mmol), was stirred in TFA (2 mL) for five minutes, and then stripped of TFA using a rotary evaporator. Product was isolated by RPLC using an Isco CombiFlash liquid chromatograph eluted with 5% to 100% methanol and water, using 0.1% TFA as the modifier. The yield of Int-27 as a TFA salt was 0.157 g, 66% yield. Ion(s) found by LCMS: [M/3]+H+=940.4, [M/4]+H+=705.5. [M/5]+H+=564.8.

### Example 63—Synthesis of Int-28

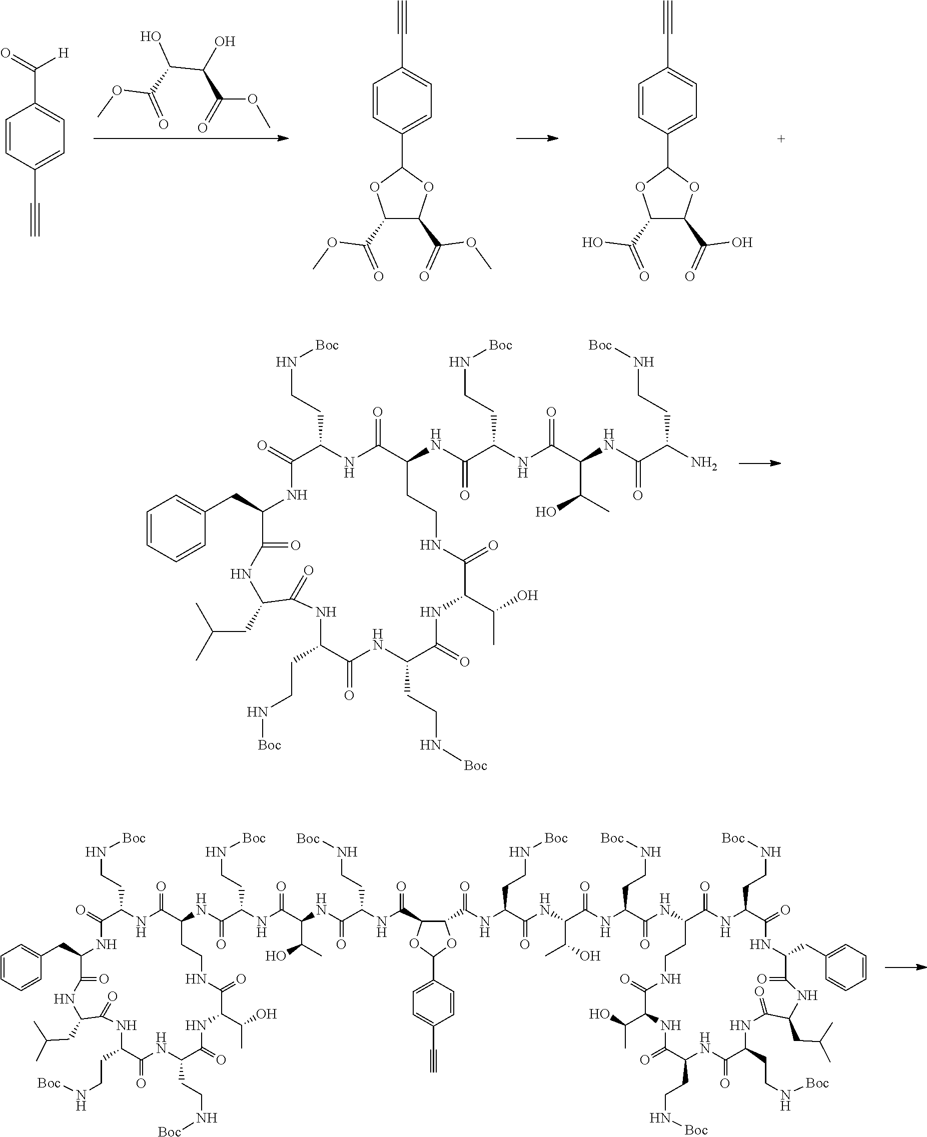

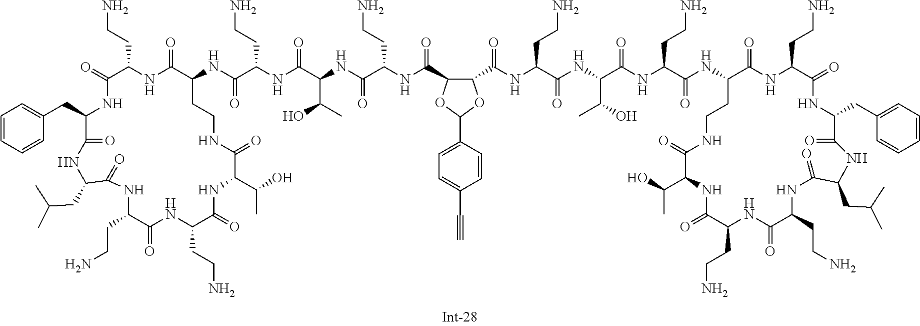 [SMILES: C#Cc1ccc(C2O[C@@H](C(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)[C@H](C(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)O2)cc1]

### Step a

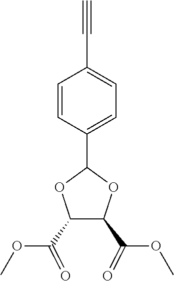

To a 60° C. stirring solution of L-dimethyltartrate (1.84 g, 9.22 mmol), 4-ethynylbenzaldehyde (600 mg, 4.61 mmol) and trimethylorthoformate (1.01 mL, 9.22 mmol), was added methane sulfonic acid (0.021 mL, 0.323 mmol) and the temperature was raised to 100° C. After 2 h, the reaction was cooled to room temperature and diluted with 20 mL of ethyl acetate. The organic layer was carefully added to a saturated aqueous solution of sodium bicarbonate (50 mL). Upon gas evolution ceased (bubble monitoring) the organic layer was separated and washed with water (50 mL), then dried with brine (50 mL) and solid magnesium sulfate. The resulting mixture was filtered through celite, concentrated, and purified by normal phase liquid chromatography (silica gel) using an Isco CombiFlash liquid chromatograph eluted with 1% to 25% hexanes and ethyl acetate. Yield 0.416 g of (4R,5R)-dimethyl 2-(4-ethynylphenyl)-1,3-dioxolane-4,5-dicarboxylate, 31% yield. ¹H NMR (Methanol-d₄) δ: 7.57 (d, J=8.3 Hz, 2H), 7.50 (d, J=8.3 Hz, 2H), 6.08 (s, 1H), 5.05 (d, J=3.8 Hz, 1H), 4.91 (d, J=3.8 Hz, 1H), 3.85 (s, 3H), 3.81 (s, 3H), 3.56 (s, 1H).

### Step b

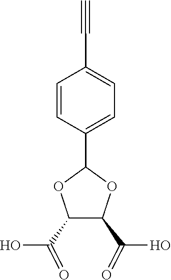 [SMILES: C#Cc1ccc(C2O[C@@H](C(=O)O)[C@H](C(=O)O)O2)cc1]

To a stirring solution of (4R,5R)-dimethyl 2-(4-ethynylphenyl)-1,3-dioxolane-4,5-dicarboxylate (0.416 g, 1.433 mmol) in tetrahydrofuran (5.0 mL) and water (5.0 mL) it was added lithium hydroxide (0.103 g, 4.299 mmol) and stirring was continued overnight. The reaction was concentrated to about half of its volume per rotatory evaporation. Ethyl acetate (15 mL) was added and subsequently a 1.0 M solution of sulfuric acid (5.0 mL), while the mixture was stirred vigorously for 10 minutes. The organic phase was separated and then washed with water (10 mL) and brine (10 mL). The organic layer was then dried with sodium sulfate, filtered, and concentrated to dryness per vacuum techniques. HPLC analysis for the residues revealed one component in high purity, so it was used in the next step without further purification. Yield 0.306 g of (4R,5R)-2-(4-ethynylphenyl)-1,3-dioxolane-4,5-dicarboxylic acid, 82% yield.

### Step c

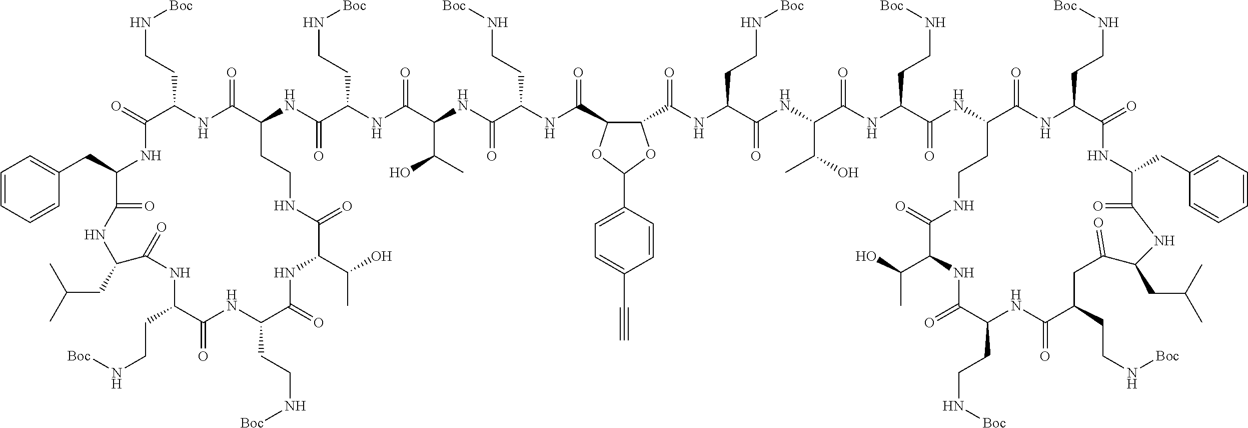

To a 0° C. stirring solution of (4R,5R)-2-(4-ethynylphenyl)-1,3-dioxolane-4,5-dicarboxylic acid (0.045 g, 0.172 mmol), Int-7a (0.537 g, 0.343 mmol, described in Example 8 step a), and DIEA (0.126 mL, 0.721 mmol) in DMF (5.0 mL), was treated with a solution of HATU (0.134 g, 0.352 mmol) in DMF (3.0 mL), dropwise. Temperature was slowly allowed to reach ambient temperature after 15 min. Upon completion, all the volatiles were rotary evaporated. The residue was purified by RPLC using an Isco CombiFlash liquid chromatograph eluted with 50% to 100% water and methanol, using no modifier. Yield 0.400 g, 69% yield. ¹H NMR diagnostic peaks (Methanol-d₄) δ: 7.61 (d, J=8.3 Hz, 2H), 7.51 (d, J=8.3 Hz, 2H), 7.27 (m, 10H), 6.14 (s, 1H), 1.44 (br s, 90H), 1.21 (t, J=5.1 Hz, 12H), 0.69 (d, J=18.8 Hz, 12H).

### Step d

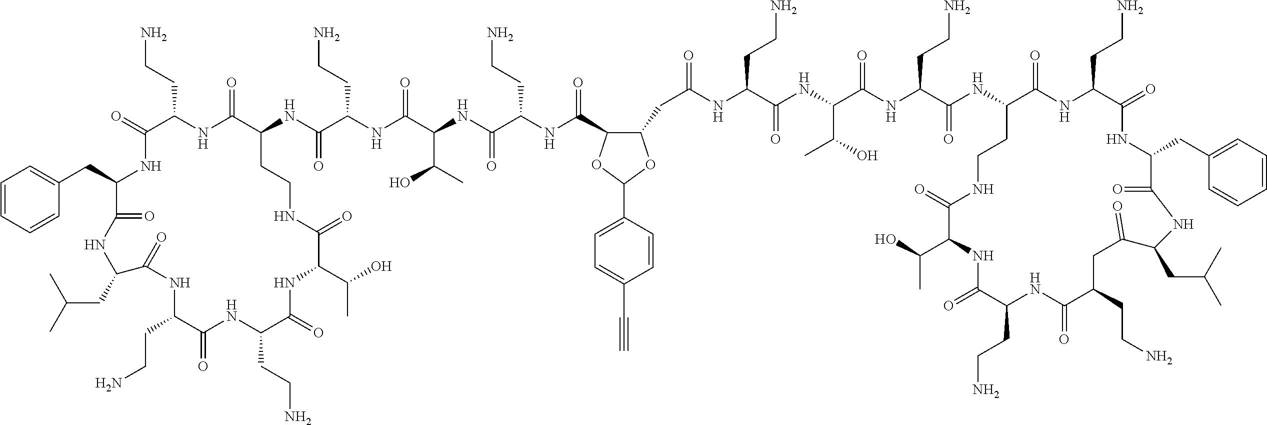 [SMILES: C#Cc1ccc(C2O[C@@H](CC(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)CC(=O)[C@H](CC(C)C)NC(=O)[C@@H](Cc4ccccc4)NC(=O)[C@H](CCN)NC3=O)[C@@H](C)O)[C@H](C(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)O2)cc1]

Product from step c (0.400 g, 0.119 mmol) was treated with dichloromethane (4.0 mL), 2-methyl-2-butene (0.25 mL), and TFA (2.0 mL) until gas evolution ceased (bubbler monitoring), then concentrated to an oil. The oil was purified by RPLC using an Isco CombiFlash liquid chromatograph eluted with 10% to 100% water and methanol, using 0.1% TFA as the modifier. Yield 0.085 g, 20% yield desired product Int-28. Ion(s) found by LCMS: (M+3H)/3=784.5, (M+4H)/4=588.8, (M+5H)/5=471.2.

### Example 64—Synthesis of Int-29

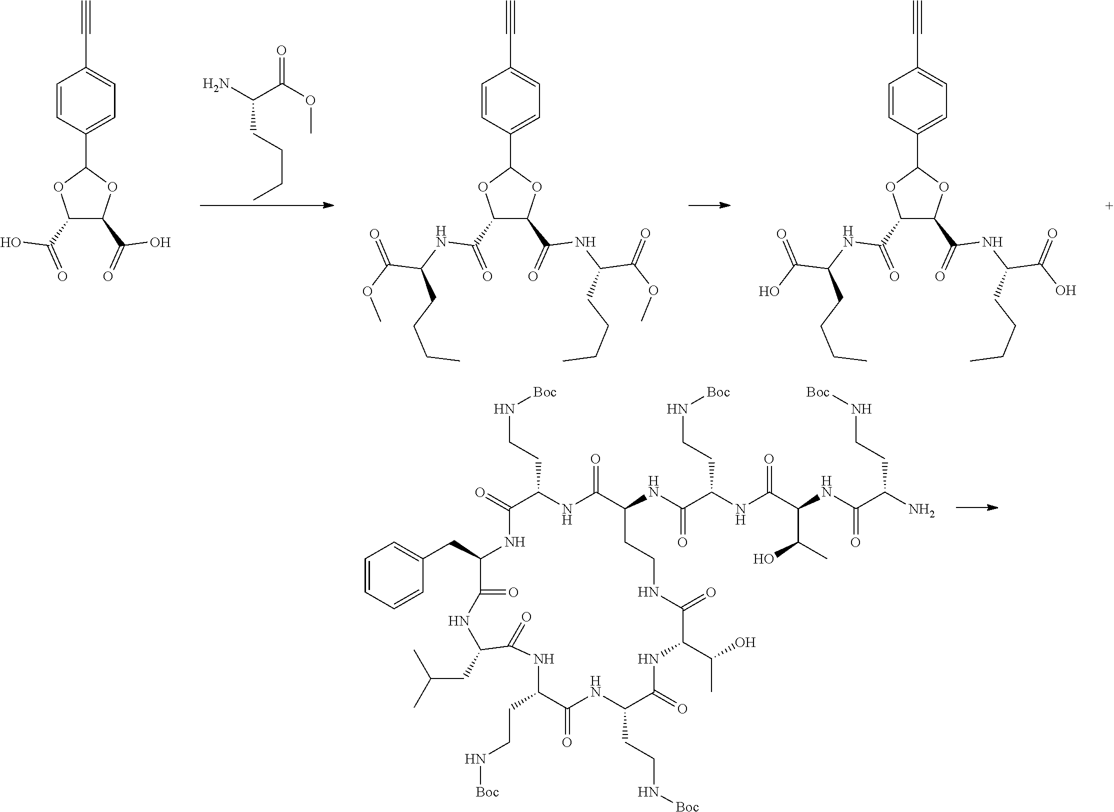

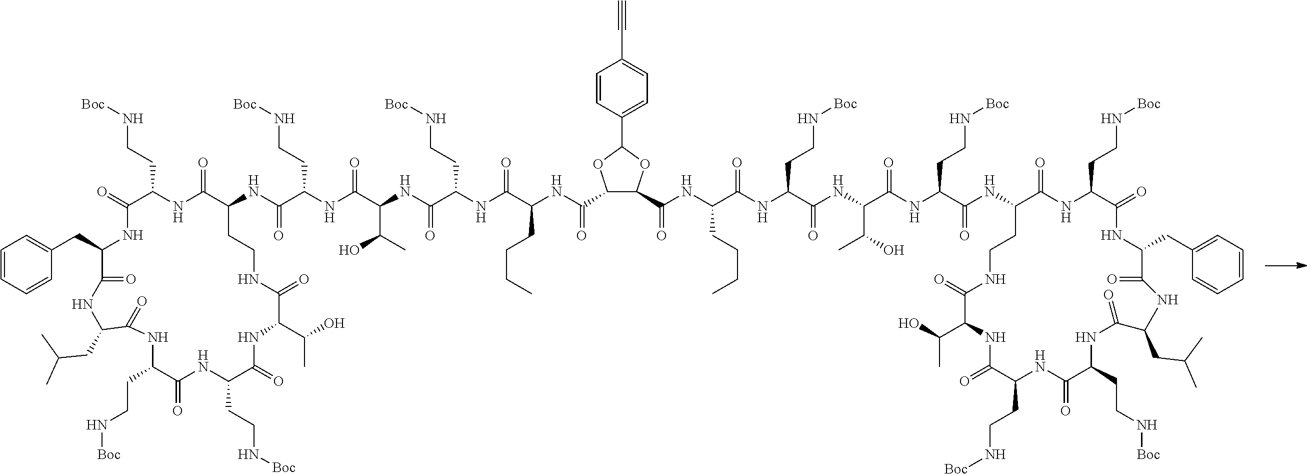

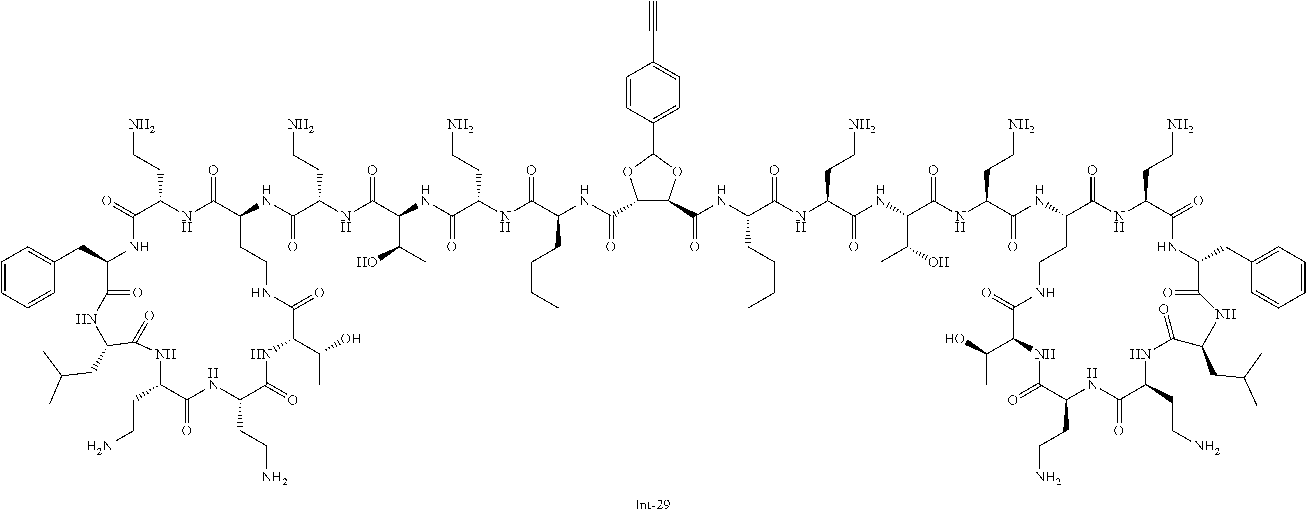 [SMILES: C#Cc1ccc(C2O[C@@H](C(=O)N[C@@H](CCCC)C(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)[C@H](C(=O)N[C@@H](CCCC)C(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)O2)cc1]

### Step a

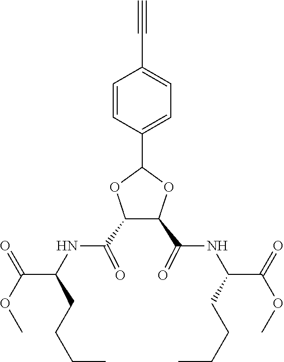 [SMILES: C#Cc1ccc(C2O[C@@H](C(=O)N[C@@H](CCCC)C(=O)OC)[C@H](C(=O)N[C@@H](CCCC)C(=O)OC)O2)cc1]

To a 0° C. stirring solution of (4R,5R)-2-(4-ethynylphenyl)-1,3-dioxolane-4,5-dicarboxylic acid (0.254 g, 0.969 mmol), L-norleucine methyl ester hydrochloride (0.370 g, 2.034 mmol), and DIEA (1.05 mL, 6.006 mmol) in DMF (5.0 mL), was treated with a solution of HATU (0.134 g, 0.352 mmol) in DMF (5.0 mL), dropwise. Temperature was slowly allowed to reach ambient after 15 min. Upon completion, all the volatiles were evaporated. The residue was purified by RPLC using an Isco CombiFlash liquid chromatograph eluted with 10% to 100% water and methanol, using no modifier. Yield 0.217 g, 43% yield of bis-(dimethyl ester). ¹H NMR (Methanol-d₄) δ: 7.62 (d, J=8.3 Hz, 2H), 7.53 (d, J=8.3 Hz, 2H), 6.13 (s, 1H), 4.89 (d, J=4.4 Hz, 1H), 4.84 (d, J=4.4 Hz, 1H), 4.49 (td, J=9.1, 8.5, 5.0 Hz, 2H), 3.75 (s, 1H), 3.74 (s, 3H), 3.58 (s, 1H), 1.79 (m, 4H), 1.34 (m, 8H), 0.91 (m, 6H).

### Step b

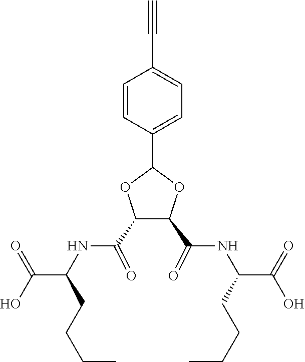 [SMILES: C#Cc1ccc(C2O[C@@H](C(=O)N[C@@H](CCCC)C(=O)O)[C@H](C(=O)N[C@@H](CCCC)C(=O)O)O2)cc1]

To a stirring solution of bis-(dimethyl ester) product from step a (0.217 g, 0.420 mmol) in tetrahydrofuran (4.0 mL) and water (1.0 mL) it was added lithium hydroxide (0.030 g, 1.260 mmol) and stirring was continued overnight. The reaction was concentrated to about 1 mL per rotatory evaporation. Dichloromethane (15 mL) was added and subsequently a 1.0 M solution of sulfuric acid (5.0 mL), while the mixture was stirred vigorously for 10 minutes. The organic phase was separated and then washed with water (10 mL) and brine (10 mL). The organic layer was then dried with sodium sulfate, filtered, and concentrated to dryness per vacuum techniques. HPLC analysis for the residues revealed one component in high purity that was used in the next step. Yield 0.207 g of dicarboxylic acid, quantitative yield.

### Step c

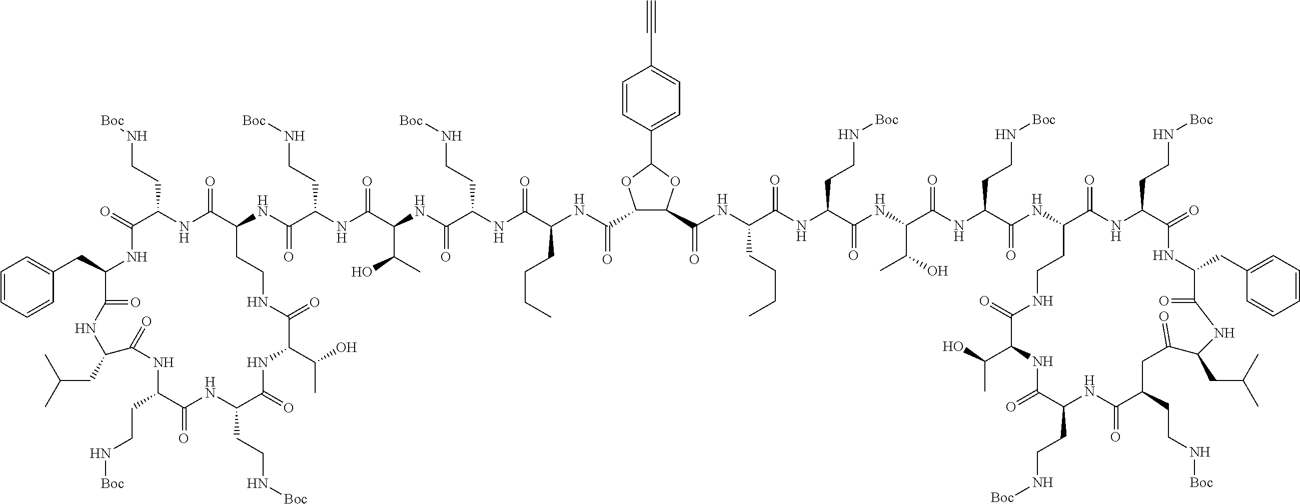

To a 0° C. stirring solution of dicarboxylic acid product from step b (0.047 g, 0.096 mmol), Int-7a (0.300 g, 0.192 mmol, described in Example 8 step a), and DIEA (0.104 mL, 0.595 mmol) in DMF (5.0 mL), was treated with a solution of HATU (0.076 g, 0.201 mmol) in DMF (3.0 mL), dropwise. Temperature was slowly allowed to reach ambient temperature after 15 min. Upon completion, all the volatiles were evaporated. The residue was purified by RPLC using an Isco CombiFlash liquid chromatograph eluted with 50% to 100% water and methanol, using no modifier. Yield 0.287 g, 84% yield. ¹H NMR for the diagnostic peaks (Methanol-d₄) δ: 7.63 (d, J=8.2 Hz, 2H), 7.53 (d, J=8.2 Hz, 2H), 7.35-7.15 (m, 10H), 6.15 (s, 1H), 4.85 (br s, 2H), 1.44 (s, 90H), 1.20 (br d, J=6.0 Hz, 12H), 0.99-0.83 (m, 6H), 0.70 (br d, J=17.6 Hz, 12H).

### Step d

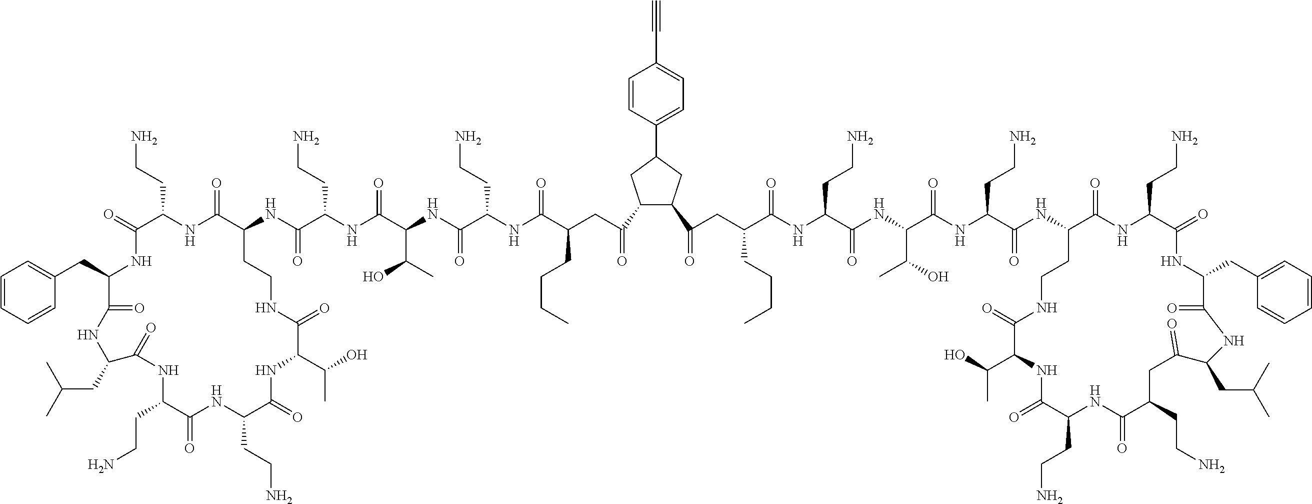 [SMILES: C#Cc1ccc(C2C[C@@H](C(=O)C[C@@H](CCCC)C(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)CC(=O)[C@H](CC(C)C)NC(=O)[C@@H](Cc4ccccc4)NC(=O)[C@H](CCN)NC3=O)[C@@H](C)O)[C@H](C(=O)C[C@@H](CCCC)C(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)C2)cc1]

Product from step c (0.287 g, 0.080 mmol) was treated with dichloromethane (4.0 mL), 2-methyl-2-butene (0.25 mL), and TFA (2.0 mL) until gas evolution ceased (bubbler monitoring), then concentrated to an oil. The oil was purified by RPLC using an Isco CombiFlash liquid chromatograph eluted with 10% to 100% water and methanol, using 0.1% TFA as the modifier. Yield 0.185 g, 62% yield of desired product Int-29. Ion(s) found by LCMS: (M+4H)/4=645.4, (M+5H)/5=516.4.

### Example 65—Synthesis of Int-30

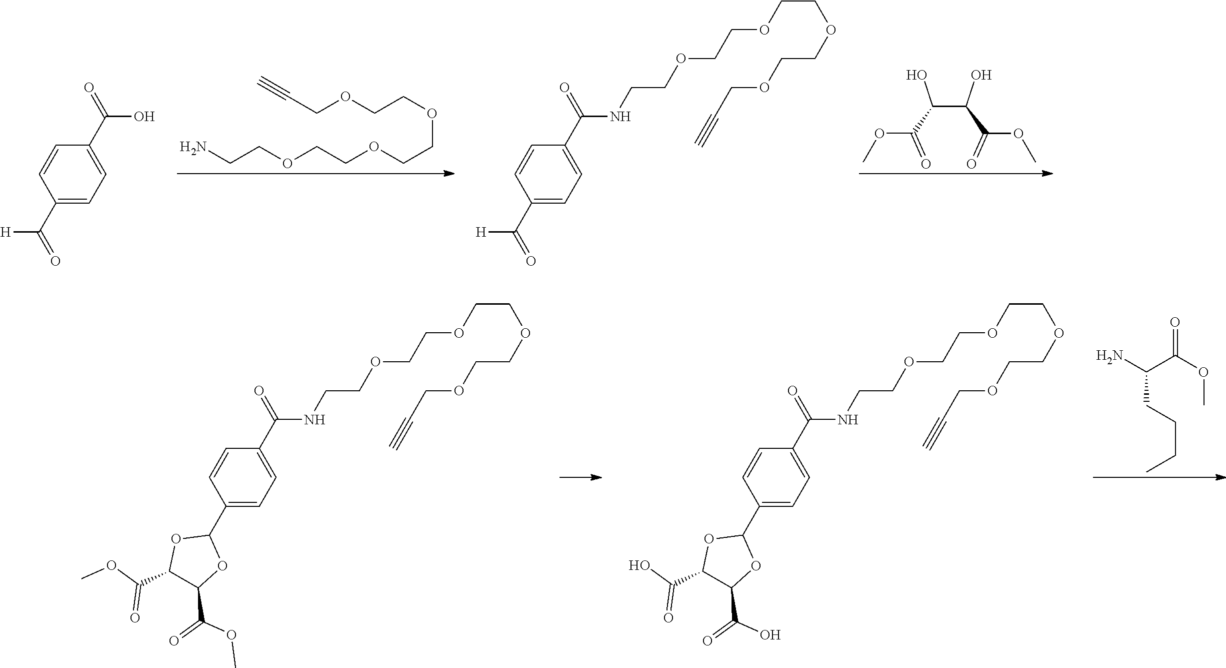

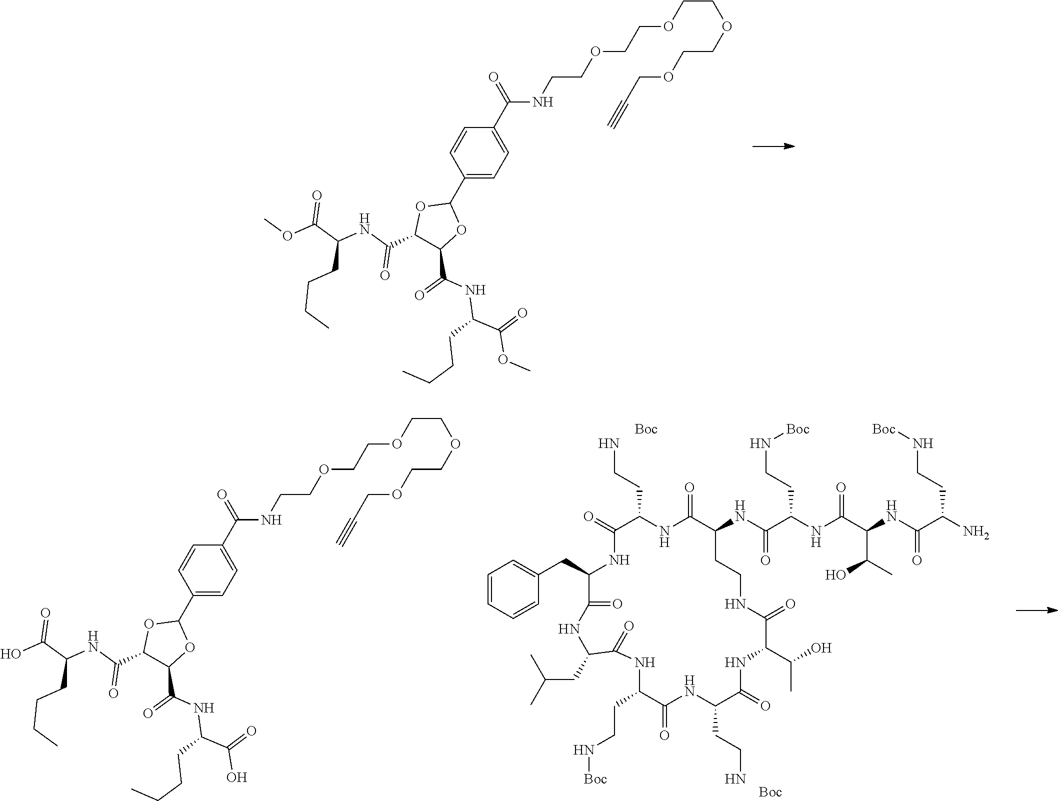

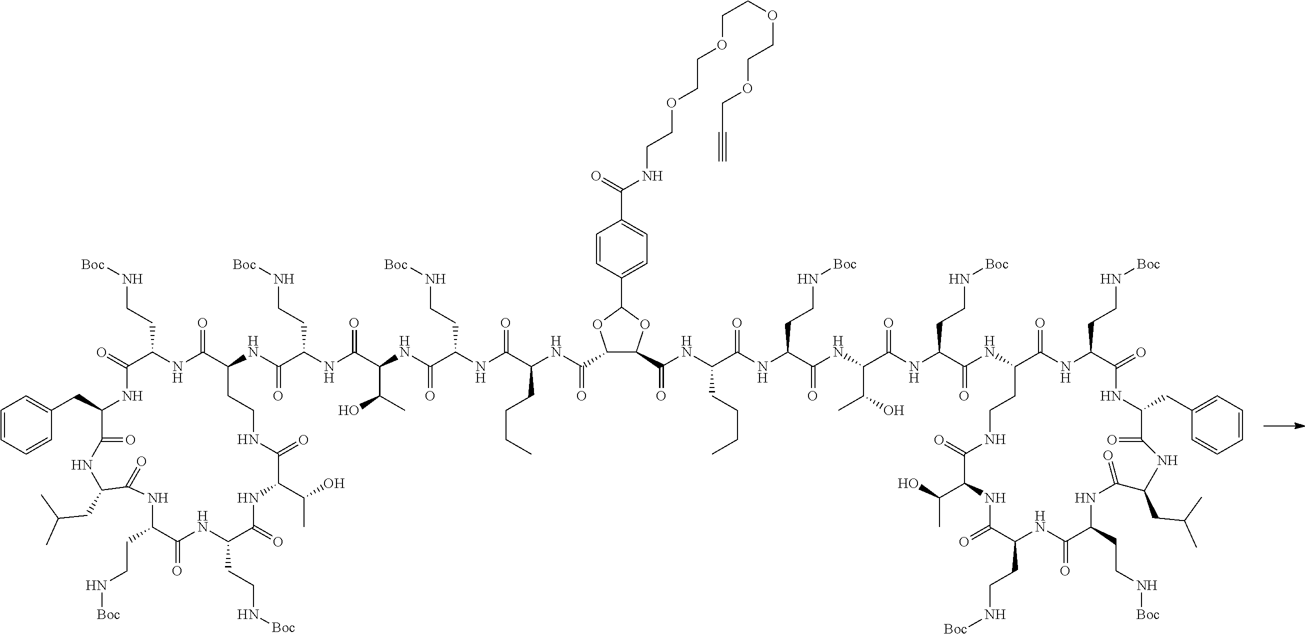

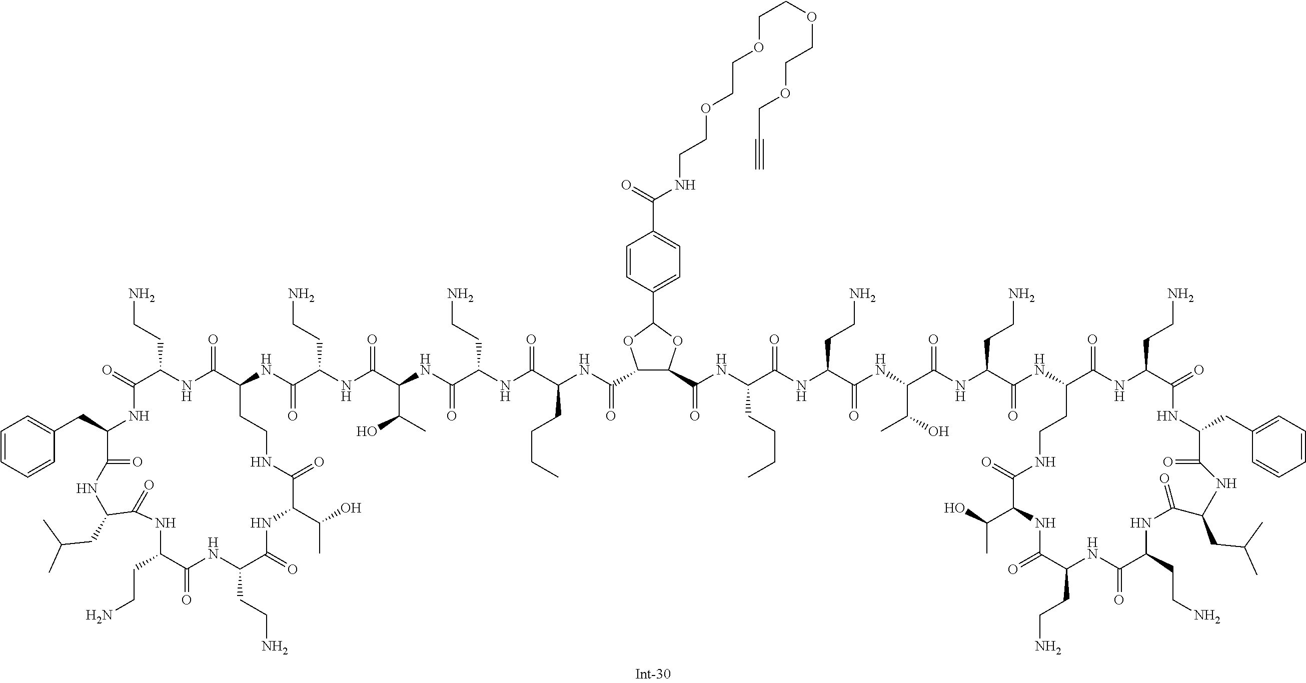 [SMILES: C#CCOCCOCCOCCOCCNC(=O)c1ccc(C2O[C@@H](C(=O)N[C@@H](CCCC)C(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)[C@H](C(=O)N[C@@H](CCCC)C(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)O2)cc1]

### Step a

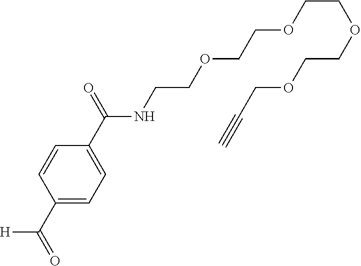 [SMILES: C#CCOCCOCCOCCOCCNC(=O)c1ccc(C=O)cc1]

To a 0° C. stirring solution of 4-formylbenzoic acid (0.260 g, 1.729 mmol), propargyl-PEG4-amine (0.400 g, 0.729 mmol), and DIEA (0.632 mL, 3.632 mmol) in DMF (2.0 mL), was treated with HATU (0.671 g, 0.1.764 mmol). Temperature was slowly allowed to reach ambient after 15 min. Upon completion, all the volatiles were evaporated. The residue was purified by RPLC using an Isco CombiFlash liquid chromatograph eluted with 10% to 100% water and methanol, using no modifier. Yield 0.570 g of N-(2-(2-(2-(2-(prop-2-ynyloxy)ethoxy)ethoxy)ethoxy)ethyl)-4-formylbenzamide, 91% yield. ¹H NMR (Methanol-d₄) δ: 10.07 (s, 1H), 8.01 (s, 4H), 4.16 (d, J=2.4 Hz, 2H), 3.76-3.51 (m, 16H), 2.84 (t, J=2.4 Hz, 1H).

### Step b

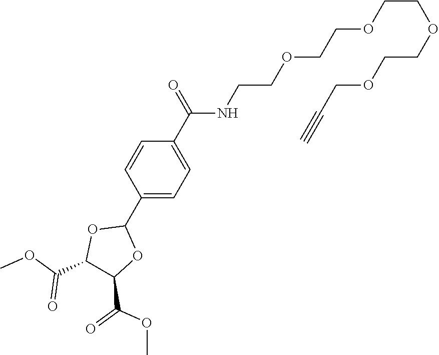 [SMILES: C#CCOCCOCCOCCOCCNC(=O)c1ccc(C2O[C@@H](C(=O)OC)[C@H](C(=O)OC)O2)cc1]

To a 60° C. stirring solution of L-dimethyltartrate (0.838 g, 4.706 mmol), N-(2-(2-(2-(2-(prop-2-ynyloxy)ethoxy)ethoxy)ethoxy)ethyl)-4-formylbenzamide (0.570 mg, 1.568 mmol) and trimethyl orthoformate (1.03 mL, 9.412 mmol), it was added methane sulfonic acid (0.005 mL, 0.078 mmol) and the temperature was raised to 100° C. After 18 h, the reaction was cooled to room temperature and diluted with 20 mL of ethyl acetate. The organic layer was carefully added to a saturated aqueous solution of sodium bicarbonate (50 mL). Upon gas evolution ceased (bubble monitoring) the organic layer was separated and washed with water (50 mL), then dried with brine (50 mL) and solid magnesium sulfate. The resulting mixture was filtered through celite, concentrated, and purified by RPLC using an Isco CombiFlash liquid chromatograph eluted with 25% to 100% water and methanol, using no modifier. Yield 0.360 g of (4R,5R)-dimethyl 2-(4-(2-(2-(2-(2-(prop-2-ynyloxy)ethoxy)ethoxy)ethoxy)ethylcarbamoyl)phenyl)-1,3-dioxolane-4,5-dicarboxylate, 44% yield. ¹H NMR (Methanol-d₄) δ: 8.56 (br s, 1H), 7.87 (d, J=8.4 Hz, 2H), 7.69 (d, J=8.4 Hz, 2H), 6.14 (s, 1H), 5.07 (d, J=3.7 Hz, 1H), 4.94 (d, J=3.7 Hz, 1H), 4.15 (d, J=2.4 Hz, 2H), 3.86 (s, 3H), 3.81 (s, 3H), 3.72-3.50 (m, 16H), 2.84 (t, J=2.4 Hz, 1H).

### Step c

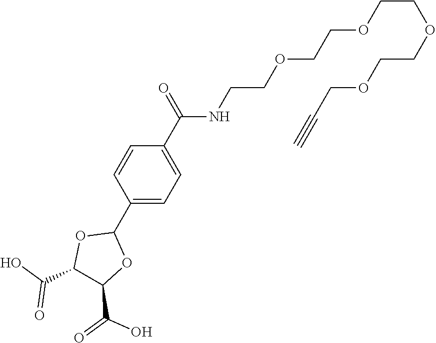 [SMILES: C#CCOCCOCCOCCOCCNC(=O)c1ccc(C2O[C@@H](C(=O)O)[C@H](C(=O)O)O2)cc1]

To a stirring solution of (4R,5R)-dimethyl 2-(4-(2-(2-(2-(2-(prop-2-ynyloxy)ethoxy)ethoxy)ethoxy)ethylcarbamoyl)phenyl)-1,3-dioxolane-4,5-dicarboxylate (0.360 g, 0.688 mmol) in tetrahydrofuran (5.0 mL) and water (2.5 mL) it was added lithium hydroxide (0.066 g, 2.751 mmol) and stirring was continued overnight. The reaction was concentrated to about 2.5 mL per rotatory evaporation. Ethyl acetate (15 mL) was added and subsequently a 1.0 M solution of sulfuric acid (4.0 mL), while the mixture was stirred vigorously for 10 minutes. The organic phase was separated and then washed with water (10 mL) and brine (10 mL). The organic layer was then dried with sodium sulfate, filtered, and concentrated to dryness. HPLC analysis revealed one component in high purity, that was used in next step without further purification. Yield 0.342 g of (4R,5R)-2-(4-(2-(2-(2-(2-(prop-2-ynyloxy)ethoxy)ethoxy)ethoxy)ethylcarbamoyl)phenyl)-1,3-dioxolane-4,5-dicarboxylic acid, quantitative yield. ¹H NMR (Methanol-d₄) δ: 7.87 (d, J=8.2 Hz, 2H), 7.71 (d, J=8.2 Hz, 2H), 6.14 (s, 1H), 4.98 (d, J=3.9 Hz, 1H), 4.87 (d, J=3.9 Hz, 1H), 4.16 (d, J=2.4 Hz, 2H), 3.73-3.52 (m, 16H), 2.83 (t, J=2.4 Hz, 1H).

### Step d

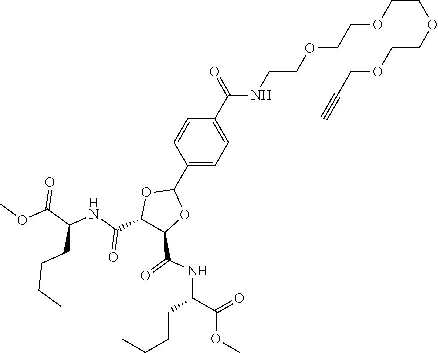 [SMILES: C#CCOCCOCCOCCOCCNC(=O)c1ccc(C2O[C@@H](C(=O)N[C@@H](CCCC)C(=O)OC)[C@H](C(=O)N[C@@H](CCCC)C(=O)OC)O2)cc1]

To a 0° C. stirring solution of (4R,5R)-2-(4-(2-(2-(2-(2-(prop-2-ynyloxy)ethoxy)ethoxy)ethoxy)ethylcarbamoyl)phenyl)-1,3-dioxolane-4,5-dicarboxylic acid (0.400 g, 0.807 mmol), L-norleucine methyl ester hydrochloride (0.246 g, 1.695 mmol), and DIEA (0.703 mL, 4.036 mmol) in DMF (5.0 mL), was treated with a solution of HATU (0.645 g, 1.695 mmol) in DMF (5.0 mL), dropwise. Temperature was slowly allowed to reach ambient temperature after 15 min. Upon completion, all the volatiles were evaporated. The residue was purified by RPLC using an Isco CombiFlash liquid chromatograph eluted with 10% to 100% water and methanol, using no modifier. Yield 0.328 g, 54% yield of bis-(dimethyl ester). ¹H NMR (Methanol-d₄) δ: 7.91 (d. J=8.3 Hz, 2H), 7.75 (d, J=8.3 Hz, 2H), 6.18 (s, 1H), 4.93 (d, J=4.5 Hz, 1H), 4.86 (d, J=4.5 Hz, 1H), 4.50 (td, J=8.1, 7.6, 5.2 Hz, 2H), 4.16 (d, J=2.4 Hz, 2H), 3.76 (s, 3H), 3.74 (s, 3H), 3.70-3.51 (m, 16H), 2.84 (t, J=2.4 Hz, 1H), 1.97-1.63 (m, 4H), 1.46-1.22 (m, 8H), 0.91 (dt, J=9.5, 7.1 Hz, 6H)

### Step e

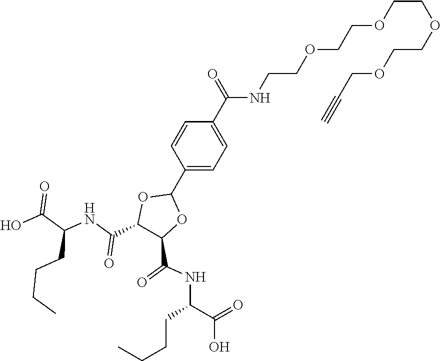 [SMILES: C#CCOCCOCCOCCOCCNC(=O)c1ccc(C2O[C@@H](C(=O)N[C@@H](CCCC)C(=O)O)[C@H](C(=O)N[C@@H](CCCC)C(=O)O)O2)cc1]

To a stirring solution of bis-(dimethyl ester) product from step d (0.328 g, 0.437 mmol) in tetrahydrofuran (5.0 mL) and water (2.5 mL) it was added lithium hydroxide (0.031 g, 1.312 mmol) and stirring was continued overnight. The reaction was concentrated to about 2.5 mL per rotatory evaporation. Ethyl acetate (15 mL) was added and subsequently a 1.0 M solution of sulfuric acid (4.0 mL), while the mixture was stirred vigorously for 10 minutes. The organic phase was separated and then washed with water (10 mL) and brine (10 mL). The organic layer was then dried with sodium sulfate, filtered, and concentrated to dryness. HPLC analysis revealed one component in high purity that was used in the next step without further purification. Yield 0.325 g of dicarboxylic acid, quantitative yield.

### Step f

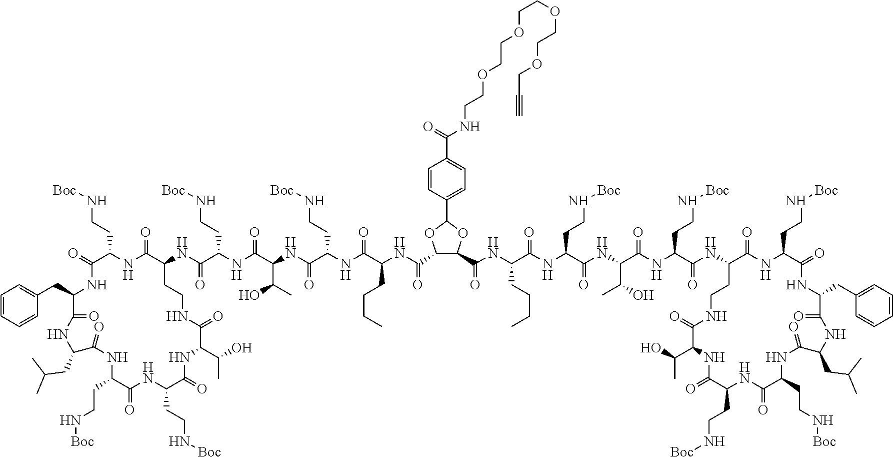

To a 0° C. stirring solution of dicarboxylic acid product from step e (0.058 g, 0.080 mmol), Int-7a (0.250 g, 0.160 mmol, described in Example 8 step a), and DIEA (0.070 mL, 0.400 mmol) in DMF (3.0 mL), was treated with a solution of HATU (0.063 g, 0.168 mmol) in DMF (1.5 mL), dropwise. Temperature was allowed to reach ambient temperature over a 15 min period. Upon completion, all the volatiles were evaporated. The residue was purified by RPLC using an Isco CombiFlash liquid chromatograph eluted with 50% to 100% water and methanol, using no modifier. Yield 0.232 g, 76% yield. ¹H NMR (Methanol-d₄) δ: 7.90 (d, J=7.9 Hz, 2H), 7.73 (d, J=7.9 Hz, 2H), 7.25 (m, 10H), 6.19 (s, 1H), 3.64 (br d, J=7.8 Hz, 16H), 2.74 (br s, 1H), 1.43 (br d, J=2.7 Hz, 90H), 1.19 (br d, J=6.2 Hz, 12H), 0.89 (br d, J=7.9 Hz, 6H), 0.70 (br d, J=18.6 Hz, 12H).

### Step g. Deprotection to Give Int-30

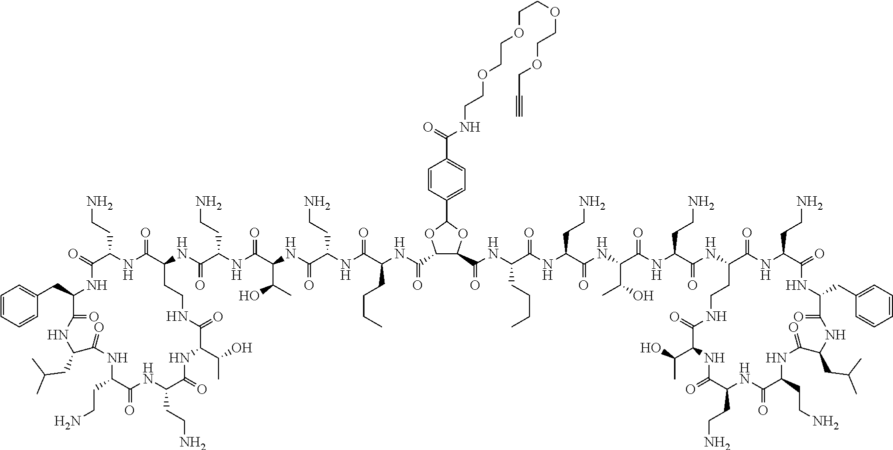 [SMILES: C#CCOCCOCCOCCOCCNC(=O)c1ccc(C2O[C@@H](C(=O)N[C@@H](CCCC)C(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)[C@H](C(=O)N[C@@H](CCCC)C(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)O2)cc1]

Product from the previous step (0.232 g, 0.061 mmol) was treated with dichloromethane (4.0 mL), 2-methyl-2-butene (0.25 mL), and TFA (2.0 mL) until gas evolution ceased (bubbler monitoring), then concentrated to an oil. The oil was purified by RPLC using an Isco CombiFlash liquid chromatograph eluted with 10% to 100% water and methanol, using 0.1% TFA as the modifier. Yield of Int-30 was 0.208 g, 86%. Ion(s) found by LCMS: (M+4H)/4=703.5, (M+5H)/5=563.2.

### Example 66—Synthesis of Int-31

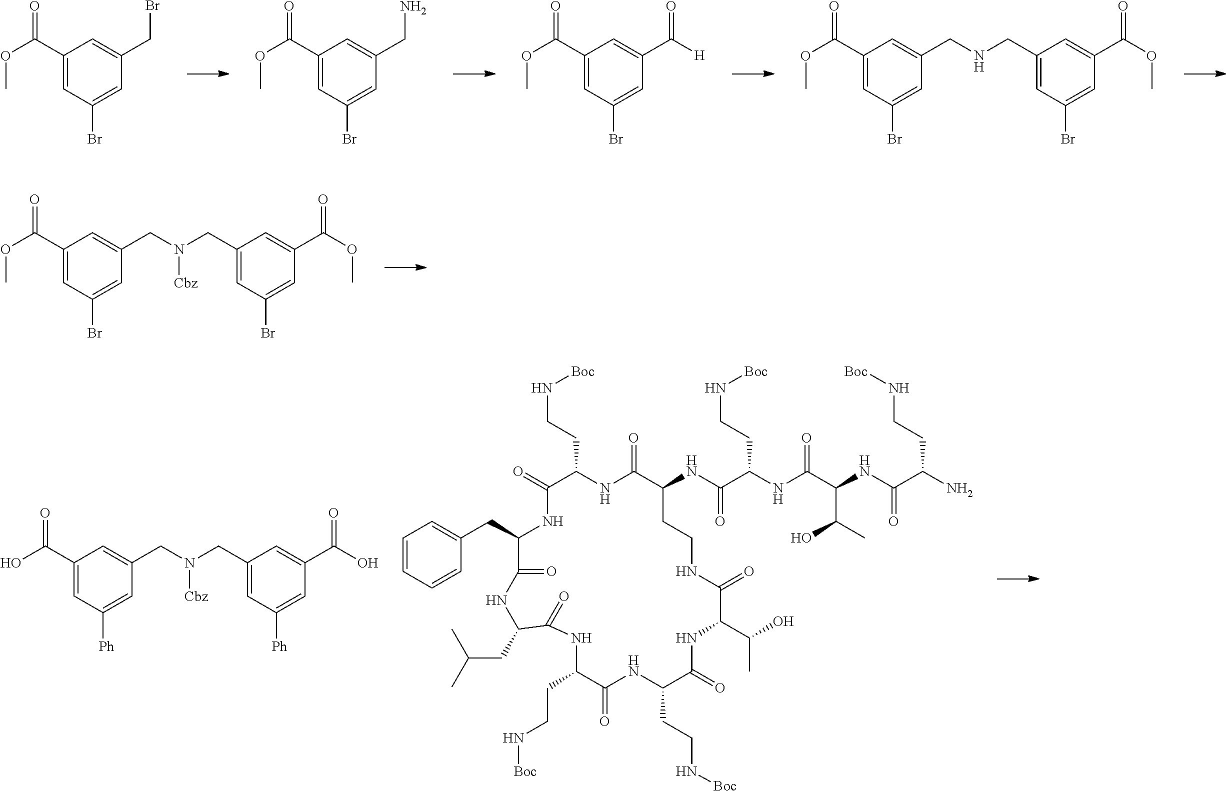

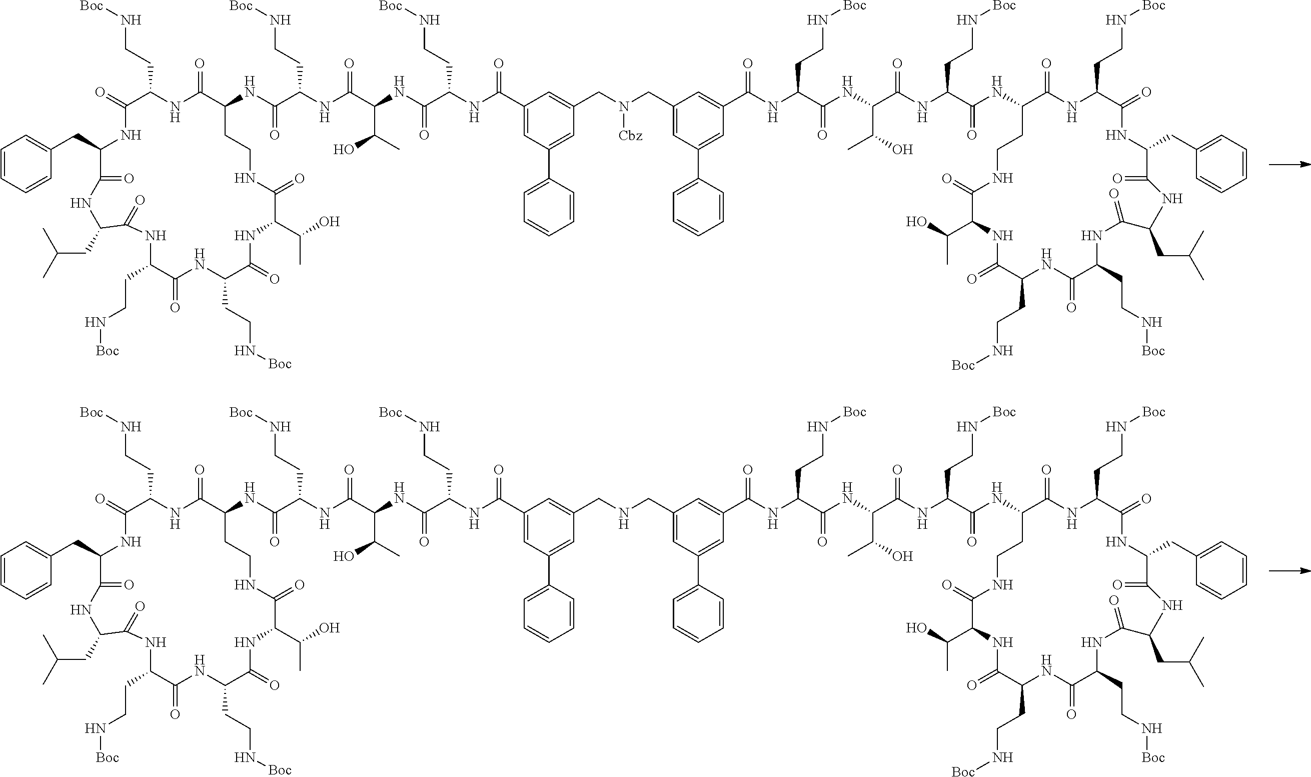

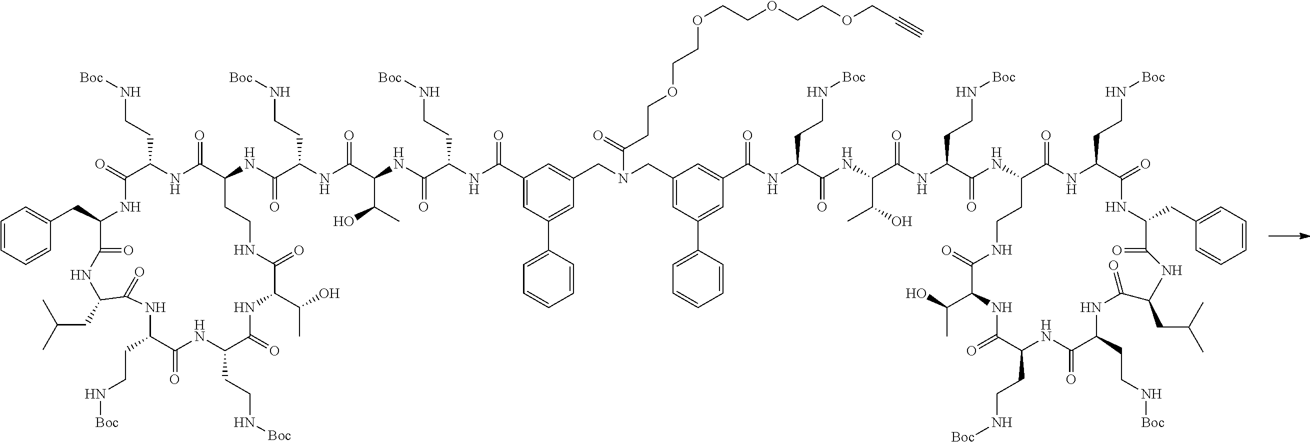

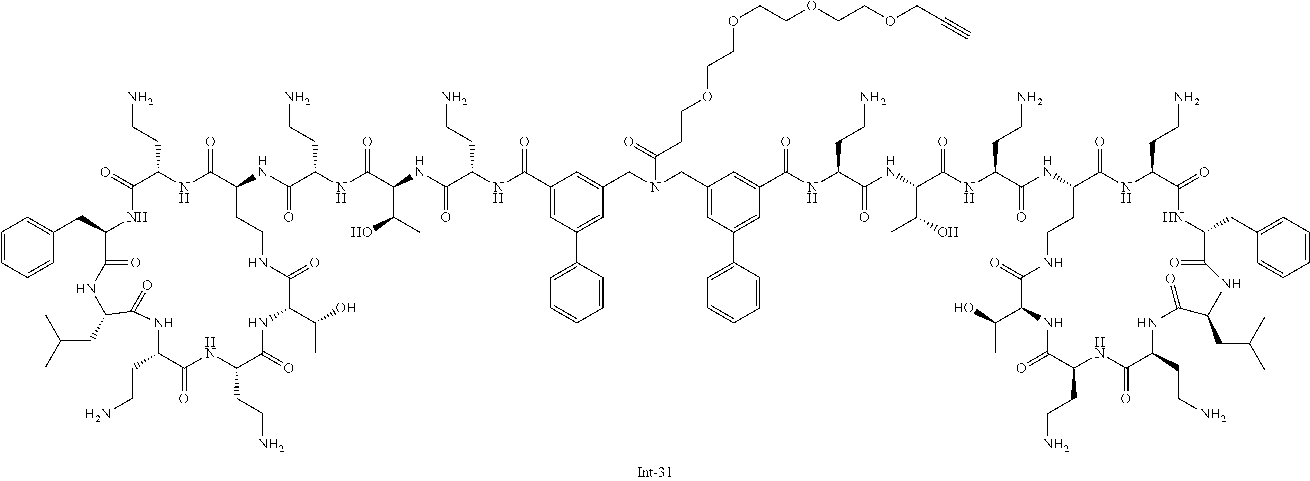 [SMILES: C#CCOCCOCCOCCOCCC(=O)N(Cc1cc(C(=O)N[C@@H](CCN)C(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)cc(-c2ccccc2)c1)Cc1cc(C(=O)N[C@@H](CCN)C(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)cc(-c2ccccc2)c1]

### Step a

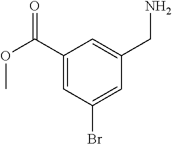

In a sealed tube, a stirring mixture of methyl 3-bromo-5-(bromomethyl)benzoate (3.08 g, 10.00 mmol) and sodium azide (0.715 g, 11.00 mmol) in DMF (10 mL) was heated at 80° C. for 1 h, when HPLC analysis showed full conversion of the bromide. All the volatiles were evaporated. The residue was taken up in DCM (40 mL) and water (40 mL). The water layer was washed with additional DCM (2×30 mL). The combined organic layers were dried with brine and solid sodium sulfate. Upon filtration, all the volatiles were evaporated, affording 2.73 g of the crude azide derivative in high purity, as confirmed per ¹H-NMR and LC-MS analysis. ¹H NMR (DMSO-d₆) δ: 8.03 (t, J=1.6 Hz, 1H), 7.98-7.93 (m, 1H), 7.90 (t, J=1.5 Hz, 1H), 4.60 (s, 2H), 3.88 (s, 3H). This azide (2.73 g) was dissolved under stirring in THF (30 mL) and water (2 mL), and triphenylphosphine (3.148 g, 12.00 mmol) was added. The reaction was stirred at room temperature overnight, allowing gas evolution through a bubbler. All the volatiles were evaporated per vacuum techniques. The desired product was isolated by normal phase liquid chromatography using an Isco CombiFlash liquid chromatograph eluted with 1% to 20% hexanes and ethylacetate. From the desired fractions, the desired compound was obtained still contaminated with triphenylphenylphosphine oxide. This material was therefore dissolved in DCM (50 mL) and extracted an acidic solution (12 mL of 1.0 M H₂SO₄ and 30 mL of water). The water layer was added dropwise to a vigorously stirring solution of saturated sodium bicarbonate (60 mL) and stirring was continued overnight. The obtained mixture was extracted with DCM (3×30 mL). The combined organic layers were dried with brine and solid sodium sulfate. Upon filtration, all the volatiles were evaporated per vacuum techniques, affording 1.31 g, 54% of 3-(aminomethyl)-5-bromobenzoate. Ions found by LCMS: (M+H)+=244.0; 248.0.

### Step b

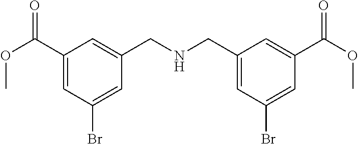 [SMILES: COC(=O)c1cc(Br)cc(CNCc2cc(Br)cc(C(=O)OC)c2)c1]

A solution of methyl 3-(aminomethyl)-5-bromobenzoate (1.306 g, 5.350 mmol) and methyl 3-bromo-5-formylbenzoate (1.238 g, 5.096 mmol) in DCM (10 mL) was evaporated and left over high vacuum overnight. The residue was taken up in THF (30 mL) and treated under vigorous stirring with sodium triacetoxyborohydride (3.240 g, 15.29 mmol). This mixture was quenched after 18 h by the addition of a saturated aqueous solution of ammonium chloride (50 mL). The obtained mixture was extracted with DCM (3×30 mL). The combined organic layers were dried with brine and solid sodium sulfate. Upon filtration, all the volatiles were evaporated. The desired product was isolated by normal phase liquid chromatography using an Isco CombiFlash liquid chromatograph eluted with 1% to 50% hexanes and ethylacetate. Yield 1.611 g, 67% of 3-((N-(1′-(3′-carboxymethyl-5-bromo)phenylene)methylene)methyl)-5-bromobenzoate. Ions found by LCMS: (M+H)+=470.0, 472.1, 474.0.

### Step c

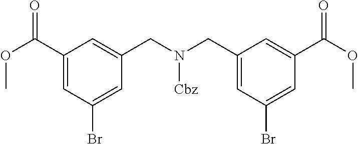 [SMILES: COC(=O)c1cc(Br)cc(CN(Cc2cc(Br)cc(C(=O)OC)c2)C(=O)OCc2ccccc2)c1]

To a 0′C stirring solution of methyl 3-((N-(1′-(3′-carboxymethyl-5-bromo)phenylene)methylene)methyl)-5-bromobenzoate (1.611 g, 3.419 mmol) and DIPEA (0.953 mL, 5.471 mmol) in THF (20 mL) it was added Cbz-Cl (0.732 mL, 5.129 mmol) dropwise. The temperature was raised to ambient temperature after 10 minutes and stirring was continued until complete disappearance of the starting amine by LC-MS analysis. All the volatiles were removed by rotatory evaporation. The desired product was isolated by normal phase liquid chromatography using an Isco CombiFlash liquid chromatograph eluted with 1% to 20% hexanes and ethylacetate. Yield 2.070 g, quantitative yield of N-Cbz protected methyl 3-((N-(1′-(3′-carboxymethyl-5-bromo)phenylene)methylene)methyl)-5-bromobenzoate. Ions found by LCMS: (M+H)+=603.0, 605.0, 607.0.

### Step d

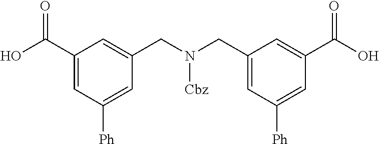 [SMILES: O=C(O)c1cc(CN(Cc2cc(C(=O)O)cc(-c3ccccc3)c2)C(=O)OCc2ccccc2)cc(-c2ccccc2)c1]

Under nitrogen, in a capped microwave reaction vessel stirring mixture of N-Cbz protected methyl 3-((N-(1′-(3′-carboxymethyl-5-bromo)phenylene)methylene)methyl)-5-bromobenzoate (0.299 g, 0.494 mmol), potassium phenyltrifluoroborate (0.200 g, 1.087 mmol), sodium carbonate (0.209 g, 1.976 mmol) and bis(triphenylphosphine)palladium (II) dichloride (0.035 g, 0.049 mmol) in MeCN (4 mL) and water (4 mL) were irradiated with microwaves for 10 min at 130° C. Upon cooling, the reaction was treated under stirring with SiliaMetS® (0.200 g, 0.240 mmol) for 1 h, and TFA was added (0.380 mL, 5.0 mmol). Upon filtration, all the volatiles were evaporated per vacuum techniques. The desired product was isolated as a dodeca-TFA salt by RPLC using an Isco CombiFlash liquid chromatograph eluted with 0% to 100% methanol and water, using TFA as the modifier. Yield 0.072 g, 25% yield of N-Cbz protected 3-((N-(1′-(3′-carboxy-5-phenyl)phenylene)methylene)methyl)-5-phenylbenzoic acid. ¹H NMR (0.500 mL Chloroform-d+0.050 mL TFA) δ: 8.18 (d, J=5.5 Hz, 2H), 7.82 (d, J=16.6 Hz, 2H), 7.60 (d, J=33.5 Hz, 2H), 7.52-7.27 (m, 15H), 5.32 (s, 2H), 4.70 (d, J=19.9 Hz, 4H).

### Step e

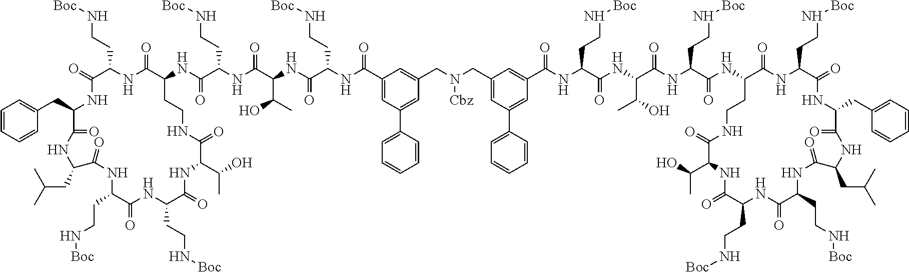

To a 0° C. stirring solution of N-Cbz protected 3-((N-(1′-(3′-carboxy-5-phenyl)phenylene)methylene)methyl)-5-phenylbenzoic acid (0.075 g, 0.131 mmol), Int-7a (0.466 g, 0.262 mmol, described in Example 8 step a), and DIEA (0.137 mL, 0.787 mmol) in DMF (5.0 mL), was treated with a solution of HATU (0.102 g, 0.269 mmol) in DMF (1.5 mL), dropwise. Temperature was slowly allowed to reach ambient temperature after 15 min. Upon completion, all the volatiles were evaporated. The residue was purified by RPLC using an Isco CombiFlash liquid chromatograph eluted with 50% to 100% water and methanol, using no modifier. Yield 0.451 g, 94% yield. ¹H NMR for the diagnostic peaks ¹H NMR (Methanol-d₄) δ: 8.12-7.09 (m, 31H), 5.25 (s, 2H), 1.41 (br d, J=10.2 Hz, 90H), 1.24-1.04 (m, 12H), 0.69 (br d, J=18.7 Hz, 12H).

### Step f

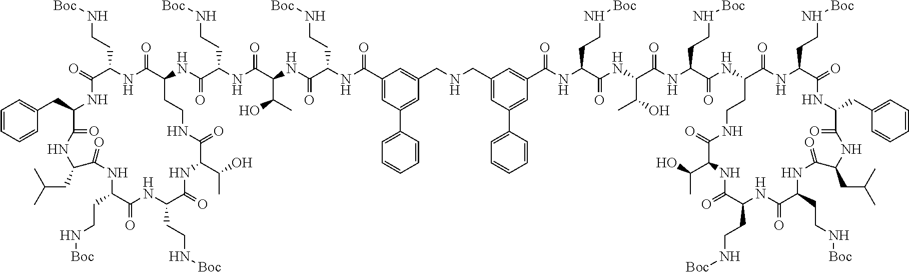

Product from step e was dissolved in methanol (25 mL) and suspended with SiliaCatPd(0)® (0.123 g, 0.025 mmol) under hydrogen atmosphere (˜1 atm). When all the starting material was consumed by HPLC analysis, the mixture was filtered over a short Celite pad, and all the volatiles were evaporated per vacuum techniques. HPLC analysis for the residues revealed one component in high purity, so it was used in the next step without further purification. Yield 0.431 g, 99% yield. ¹H NMR for the diagnostic peaks ¹H NMR (Methanol-d₄) δ: 8.07 (d, J=9.0 Hz, 2H), 7.87 (d, J=8.4 Hz, 4H), 7.74-7.70 (m, 4H), 7.55-7.11 (m, 18H), 1.42 (d, J=11.1 Hz, 90H), 1.20 (br t, J=6.7 Hz, 12H), 0.69 (br d, J=17.9 Hz, 12H)

### Step g

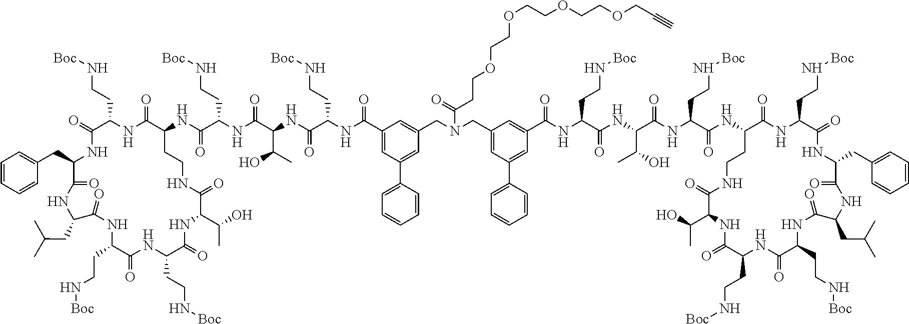

To a 0° C. stirring solution of propargyl-PEG4-acid (0.032 g, 0.122 mmol), product from step f (0.431 g, 0.122 mmol), and DIEA (0.064 mL, 0.366 mmol) in DMF (5.0 mL), was treated with a solution of HATU (0.049 g, 0.128 mmol) in DMF (1.5 mL), dropwise. Temperature was slowly allowed to reach ambient after 15 min. Upon completion, all the volatiles were evaporated per vacuum techniques. The residue was purified by RPLC using an Isco CombiFlash liquid chromatograph eluted with 50% to 100% water and methanol, using no modifier. Yield 0.326 g, 71% yield.

### Step h

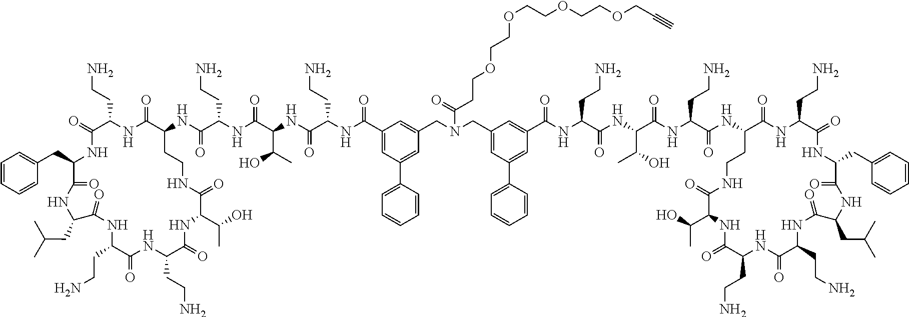 [SMILES: C#CCOCCOCCOCCOCCC(=O)N(Cc1cc(C(=O)N[C@@H](CCN)C(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)cc(-c2ccccc2)c1)Cc1cc(C(=O)N[C@@H](CCN)C(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)cc(-c2ccccc2)c1]

Product from step g (0.341 g, 0.086 mmol) was treated with dichloromethane (4.0 mL), 2-methyl-2-butene (0.25 mL), and TFA (2.0 mL) until gas evolution ceased (bubbler monitoring), then concentrated to an oil. The oil was purified by RPLC using an Isco CombiFlash liquid chromatograph eluted with 10% to 100% water and methanol, using 0.1% TFA as the modifier. Yield of Int-31 was 0.251 g, 74% yield. Ion(s) found by LCMS: (M+4H)/4=693.3, (M+5H)/5=554.8.

### Example 67—Synthesis of Int-32

The general method for the preparation of the extended Threonines is described in the following reference: David Aiker, Giles Hamblett, Laurence M. Harwood, Sarah M. Robertson, David J. Watkin and Eleri Williams, Tetrahedron 54 (1998) 6089-6098.

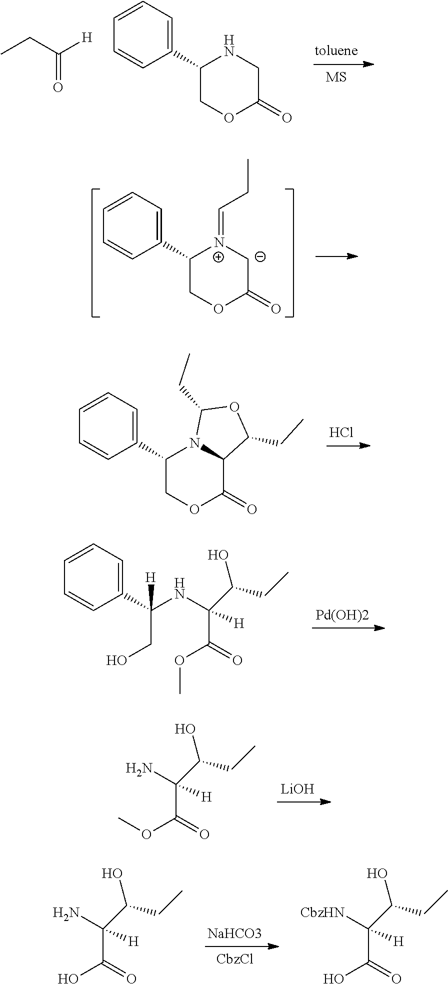

### Step a

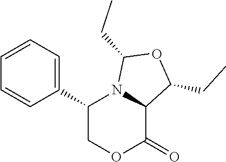 [SMILES: CC[C@@H]1O[C@H](CC)[C@H]2C(=O)OC[C@H](c3ccccc3)N12]

Propionaldehyde (7.4 g, 127 mmol, 3 eq.) was added to a solution of the 5-phenyl-morpholin-2-one (7.5 g, 42 mmol, 1 eq.) in 100 mL toluene along with 15 grams of activated 4 Å sieves and a condenser. The reaction mixture was heated to reflux for 24 hours under nitrogen. The reaction was filtered and concentrated to give the crude product as an oil, which was used in the next step without any purification. Ion(s) found by LCMS: (M+H)⁺=276.2.

### Step b

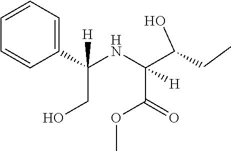 [SMILES: CC[C@@H](O)[C@H](N[C@H](CO)c1ccccc1)C(=O)OC]

To the crude material from step a in methanol (50 mL) was added 2 M HCl (10 mL). The reaction mixture was heated to reflux under nitrogen until LCMS indicated the absence of starting material and the presence of a new compound (1-2 h). The solvent was removed in vacuo to yield a yellow oil, which was then purified by RPLC using an Isco CombiFlash liquid chromatograph eluted with 10% to 100% acetonitrile and water, using 0.1% TFA as the modifier. Yield of the product as yellow oil: 5.6 g, 42% yield in two steps. Ion(s) found by LCMS: (M+H)+=267.2.

### Step c

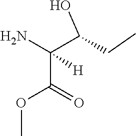

The compound from step b (1.3 g, 5 mmol) was dissolved into 30 mL methanol, Pearlman's catalyst (1.3 g) and ammonium formate (3.1 g, 10 eq.) were added to the reaction vessel and the solution degassed and heated to reflux for 4 hours. The solution was cooled and filtered to remove the catalyst. Solvent was removed in vacuo and the crude mixture was purified by RPLC using an Isco CombiFlash liquid chromatograph eluted with 10% to 100% acetonitrile and water, using 0.1% TFA as the modifier. Yield of product as yellow oil as TFA salt, 1.30 g, 100% yield. Ion(s) found by LCMS: (M+H)⁺=147.1.

### Step d

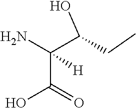

Lithium hydroxide (120 mg, 5 mmol) in 2 mL water was added into a solution of the amino acid ester from the previous step (0.3 g, 2 mmol) in 2 mL water, 2 mL methanol and 2 mL THF. The reaction was monitored by LCMS. After completion, the solution was treated with Amberlite® IRN-77, ion exchange resin to adjust to pH 1, then filtered, concentrated, and used in next step without further purification.

### Step e

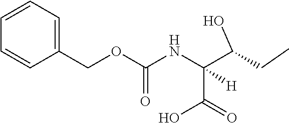 [SMILES: CC[C@@H](O)[C@H](NC(=O)OCc1ccccc1)C(=O)O]

The amino acid from the previous step (0.3 g, 2 mmol) was dissolved in 5 mL methanol and 5 mL Sat. NaHCO₃ solution, followed added CbzCl (510 mg, 3 mmol, 1.5 eq.), the solution was stirred for 1 hour then neutralized by 1N HCl. The solution was extracted with ethyl acetate and dried over Na₂SO₄. The solution was concentrated and purified by RPLC using an Isco CombiFlash liquid chromatograph eluted with 10% to 100% acetonitrile and water, using 0.1% TFA as the modifier. Yield of the product as a yellow oil, 450 mg, 92% yield. Ion(s) found by LCMS: (M+H)+=268.1.

### General Method for the Preparation of a Decapeptide Containing an Extended L-Threonine

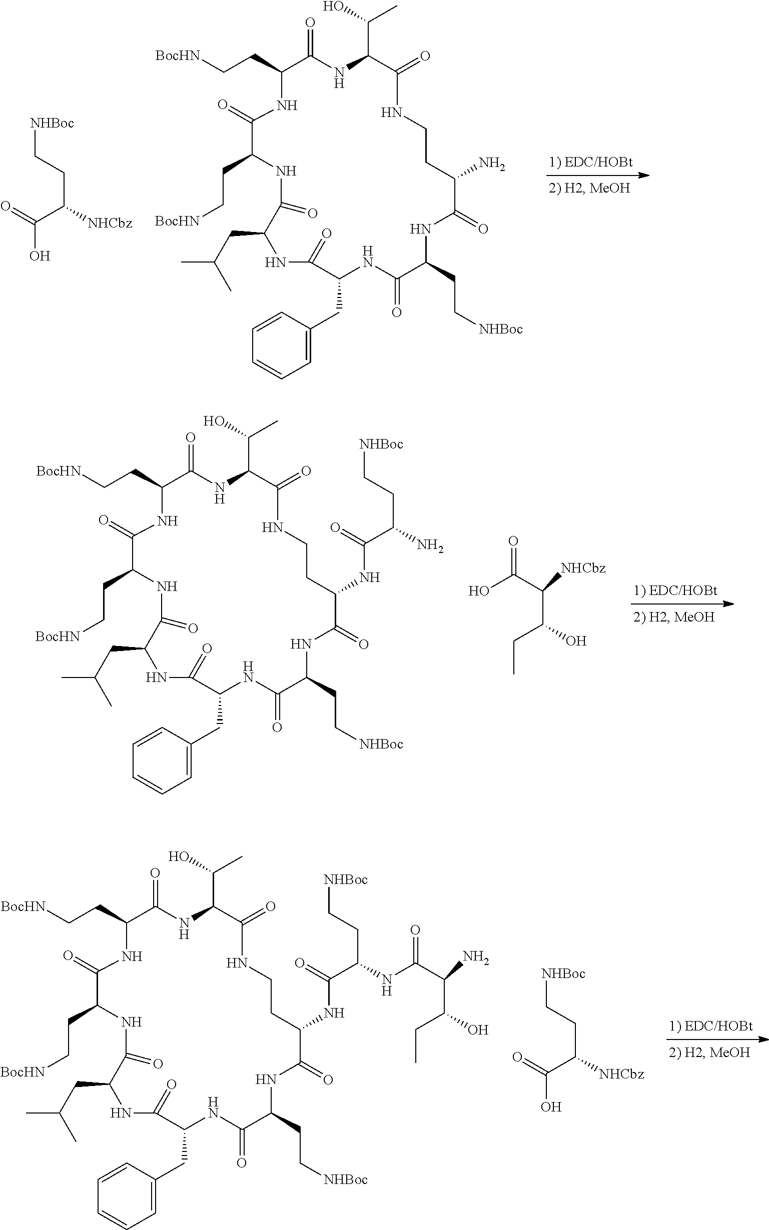

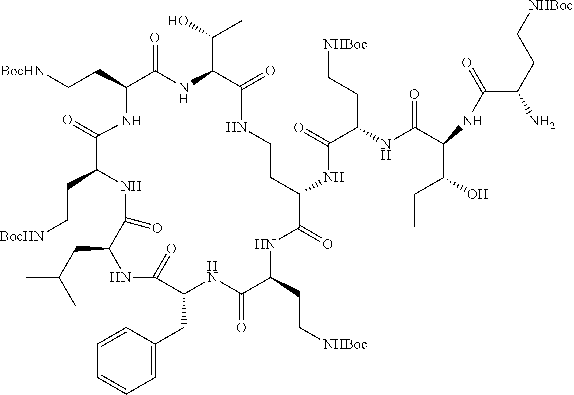 [SMILES: CC[C@@H](O)[C@H](NC(=O)[C@@H](N)CCNC(=O)OC(C)(C)C)C(=O)N[C@@H](CCNC(=O)OC(C)(C)C)C(=O)N[C@H]1CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](Cc2ccccc2)NC(=O)[C@H](CCNC(=O)OC(C)(C)C)NC1=O]

### Step f

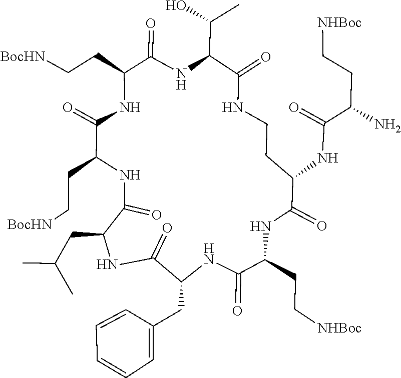

To a solution of Int-1 (2.1 g, 2 mmol, Example 2) and (2S)-2-{[(benzyloxy) carbonyl]-amino}-4-[(tert-butoxycarbonyl)amino]butanoic acid (740 mg, 2 mmol) in DMF (30 mL) was added EDC (0.6 g, 3 mmol), HOBT (0.45 g, 3 mmol), and DIEA (0.7 mL, 5 mmol) at room temperature. The solution was stirred overnight. The resulting solution was concentrated and re-dissolved into 5 mL methanol, then added drop-wise to 200 mL vigorously stirred water, this heterogeneous solution was stirred for 1 hour, then filtered with Buchner funnel with Filter paper. The collected crude product was re-dissolved into 20 mL methanol, then 1 g of 5% Pd/C was added to the above solution, and the mixture was stirred at room temperature under a hydrogen atmosphere overnight. Palladium was removed by filtration after the reaction was complete as determined by LCMS. The filtrate was concentrated and used next step without further purification. Ion(s) found by LCMS: [M/2]+H+=631.9, [M/3]+H+=421.6.

### Step g

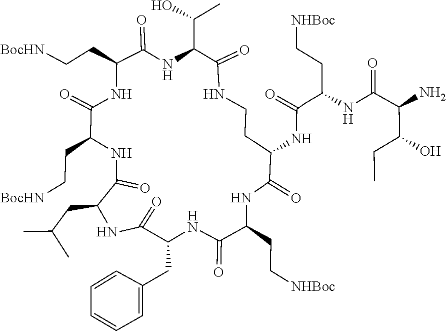 [SMILES: CC[C@@H](O)[C@H](N)C(=O)N[C@@H](CCNC(=O)OC(C)(C)C)CC(=O)N[C@H]1CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](Cc2ccccc2)NC(=O)[C@H](CCNC(=O)OC(C)(C)C)NC1=O]

To a solution of product from the previous step f and (3R)-N-[(benzyloxy)carbonyl]-3-hydroxy-L-norvaline (540 mg, 2 mmol) in DMF (20 mL) was added EDC (0.6 g, 3 mmol), HOBT (0.45 g, 3 mmol), DIEA (0.56 mL, 4 mmol) at room temperature. The solution was stirred overnight. The resulting solution was concentrated and re-dissolved into 5 mL methanol and added dropwise into 100 mL vigorously stirred water, this heterogeneous solution was stirred for 1 hour, then filtered with Buchner funnel with filter paper. The collected crude product was re-dissolved in 20 mL methanol, then treated with 1 g of 5% Pd/C. The mixture was stirred at room temperature under a hydrogen atmosphere overnight. The palladium charcoal was removed by filtration. The filtrate was concentrated and used next step without further purification. Ion(s) found by LCMS: [M/2]+H+=689.4. [M/3]+H+=459.9.

### Step h

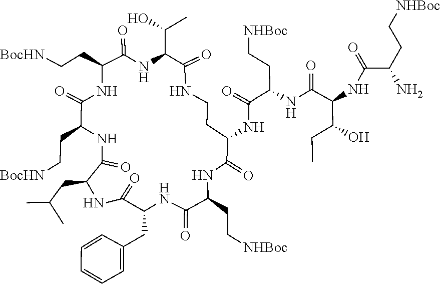 [SMILES: CC[C@@H](O)[C@H](NC(=O)[C@@H](N)CCNC(=O)OC(C)(C)C)C(=O)N[C@@H](CCNC(=O)OC(C)(C)C)CC(=O)N[C@H]1CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](Cc2ccccc2)NC(=O)[C@H](CCNC(=O)OC(C)(C)C)NC1=O]

To a solution of PMB nonapeptide-NH2 from the previous step g and (2S)-2-{[(benzyloxy) carbonyl]-amino}-4-[(tert-butoxycarbonyl)amino]butanoic acid (720 mg, 2 mmol) in DMF (20 mL) was added EDC (0.6 g, 3 mmol), HOBT (0.45 g, 3 mmol), DIEA (0.56 mL, 4 mmol) at room temperature. The solution was stirred overnight. The resulting solution was concentrated and re-dissolved into 5 mL methanol, then added drop-wise into 100 mL vigorously stirred water. This heterogeneous solution was stirred for 1 hour, then filtered with Buchner funnel with filter paper. The collected crude product was dissolved in 20 mL methanol, then treated with 1 g of 5% Pd/C. The mixture was stirred at room temperature under a hydrogen atmosphere overnight. The palladium charcoal was removed by filtration. The filtrate was concentrated and purified by RPLC using an Isco CombiFlash liquid chromatograph eluted with 10% to 100% acetonitrile and water, using 0.1% TFA as the modifier. Yield of product as a TFA salt: 2.3 g, 73% yield. Ion(s) found by LCMS: [M/2]+H+=789.5, [M/3]+H+=526.7, [M/4]+H+=395.3.

### General Method for the Preparation of the Corresponding PMB Dimer

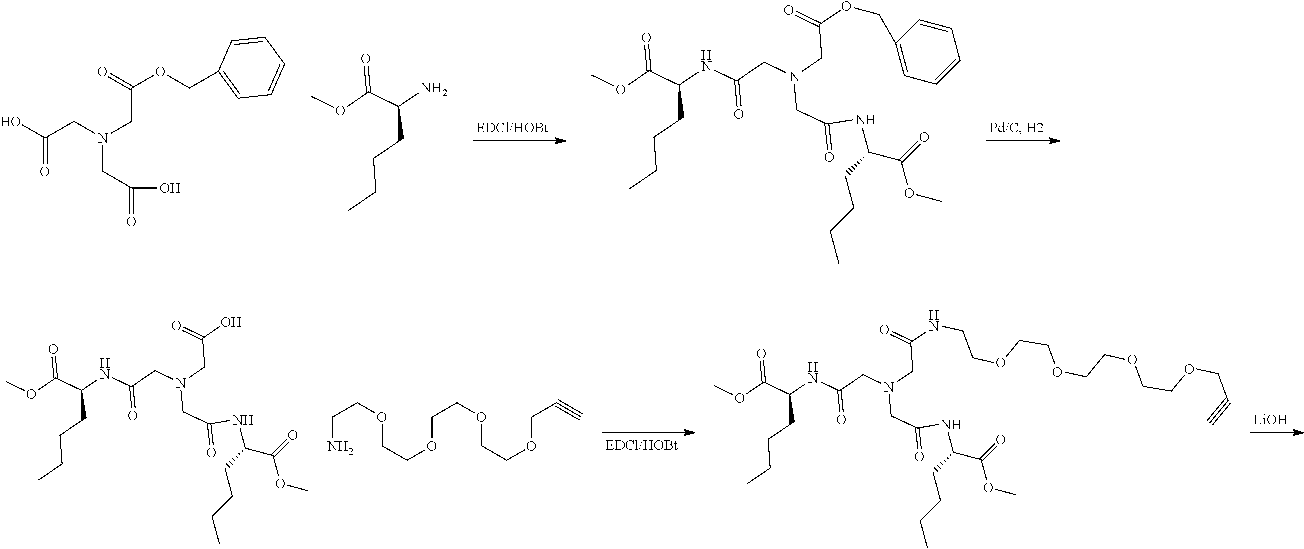

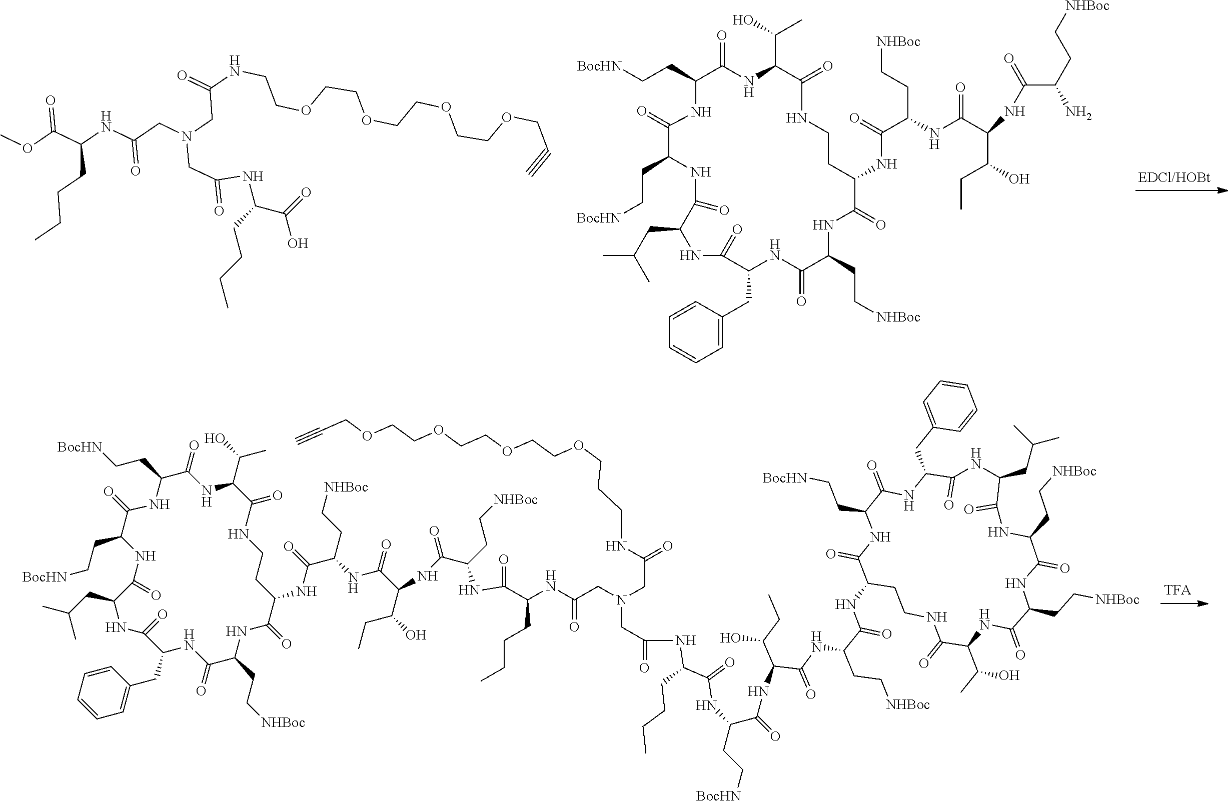

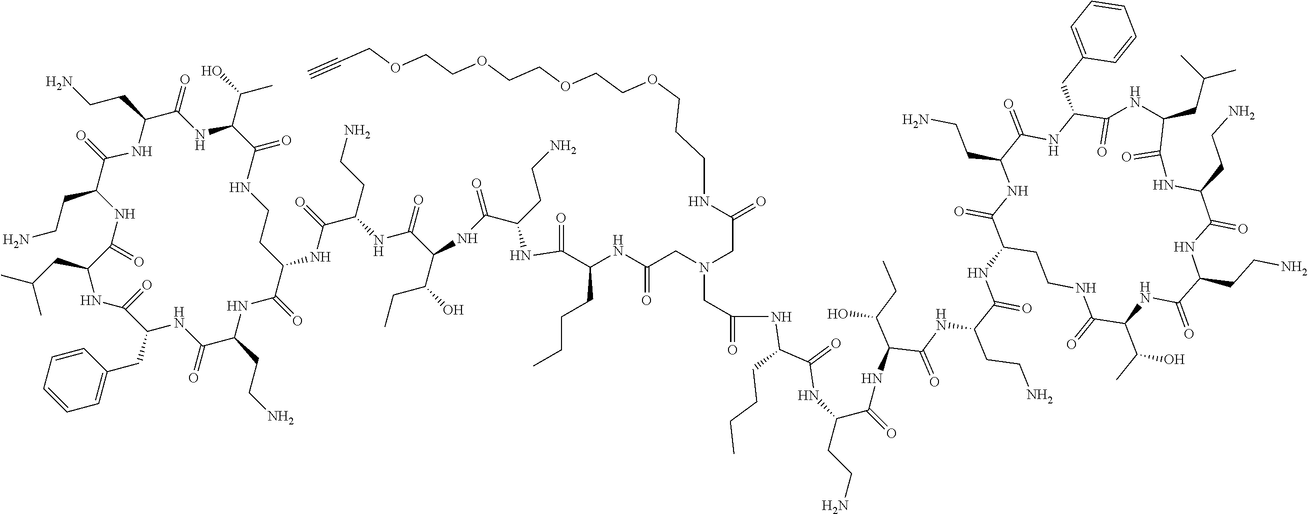 [SMILES: C#CCOCCOCCOCCOCCCNC(=O)CN(CC(=O)N[C@@H](CCCC)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CC)CC(=O)N[C@@H](CCCC)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CC]

### Step i

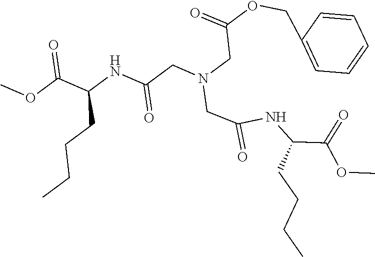

To a solution of cbz-diacetic acid (2 g, 7 mmol), and methyl L-norleucine HCl salt (2.1 g, 14.2 mmol) in DMF (40 mL) was added EDC (4.0 g, 20 mmol), HOBT (3.1 g, 20 mmol), and DIEA (4.2 mL, 30 mmol) at room temperature. The solution was stirred overnight. The resulting solution was concentrated and purified by RPLC using an Isco CombiFlash liquid chromatograph eluted with 5% to 100% acetonitrile and water, using 0.1% TFA as the modifier. Yield of the product as an oil 2.7 g, 72% yield. Ion(s) found by LCMS: (M+H)+=536.3.

### Step j

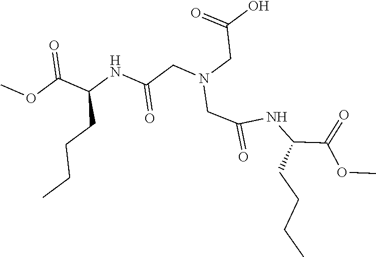

The compound from step i (536 mg, 1 mmol) was dissolved in 10 mL methanol, then 200 mg of 5% Pd/C was added to the solution. The mixture was stirred at room temperature under a hydrogen atmosphere overnight. The palladium on charcoal was filtered, concentrated, and used in next step without further purification.

### Step k

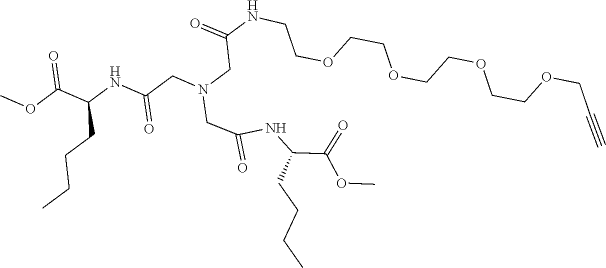 [SMILES: C#CCOCCOCCOCCOCCNC(=O)CN(CC(=O)N[C@@H](CCCC)C(=O)OC)CC(=O)N[C@@H](CCCC)C(=O)OC]

A solution of the compound from step j (450 mg, 1 mmol), propargyl-PEG4-NH₂ (230 mg, 1 mmol) in DMF (5 mL) was treated with EDC (300 mg, 1.5 mmol), HOBT (230 mg, 1.5 mmol), and DIEA (0.3 mL, 1.5 mmol) at room temperature. The solution was stirred overnight. The resulting solution was concentrated and purified by RPLC using an Isco CombiFlash liquid chromatograph eluted with 5% to 100% acetonitrile and water, using 0.1% TFA as the modifier. Yield of the product as an oil, 520 mg, 78% yield. Ion(s) found by LCMS: (M+H)+=659.4.

### Step l

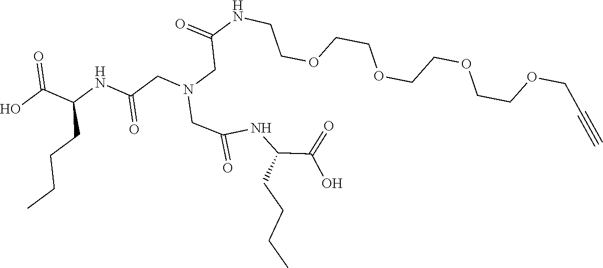 [SMILES: C#CCOCCOCCOCCOCCNC(=O)CN(CC(=O)N[C@@H](CCCC)C(=O)O)CC(=O)N[C@@H](CCCC)C(=O)O]

Lithium hydroxide (120 mg, 5 mmol) dissolved in 2 mL water was added to a solution of the amino acid ester from step k (0.65 g, 1 mmol) in 2 mL methanol and 2 mL THF. The progress of the reaction was monitored by LCMS. After completion, the solution was treated with Amberlite® IRN-77 ion exchange resin to adjust the pH to 1. The resulting solution was filtered and concentrated then used in next step without further purification. Ion(s) found by LCMS: (M+H)+=631.4.

### Step m

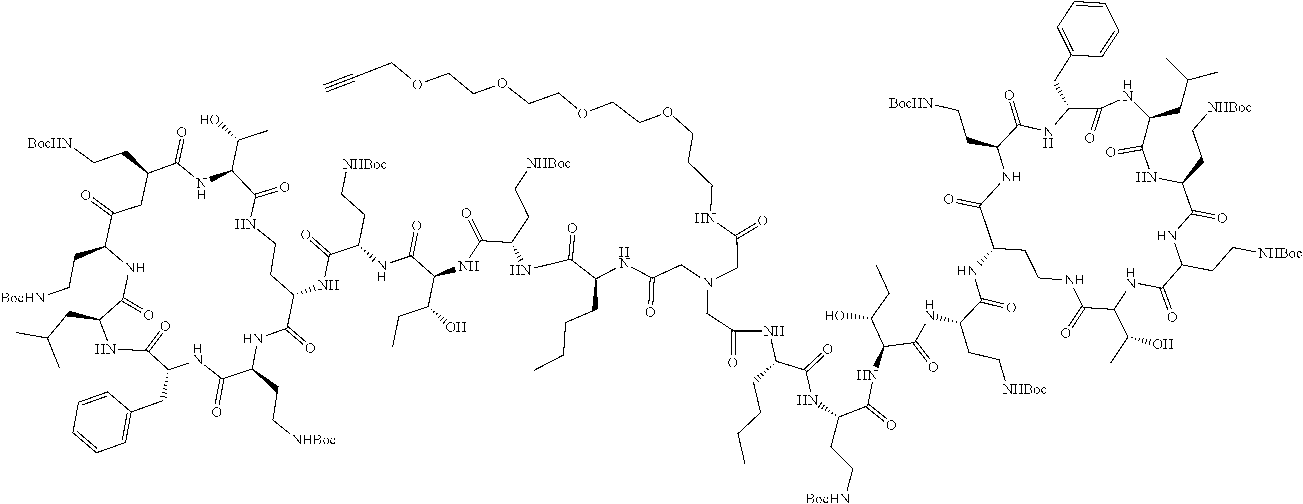

The compound from the previous step 1 (56 mg, 0.089 mmol), and penta-Boc-polymyxin B decapeptide (295 mg, 0.18 mmol, Example 8, step a) in DMF (5 mL) were added EDC (60 mg, 0.3 mmol), HOBT (45 mg, 0.3 mmol), and DIEA (0.14 mL, 1.0 mmol) at room temperature. The solution was stirred overnight. The resulting solution was concentrated and re-dissolved into 2 mL methanol. The resulting solution was added dropwise into 50 mL vigorously stirred water, this heterogeneous solution was stirred for 1 hour, then filtered with Buchner funnel with filter paper. The crude product was used in next step without further purification.

### Step n. Deprotection to Give Int-32

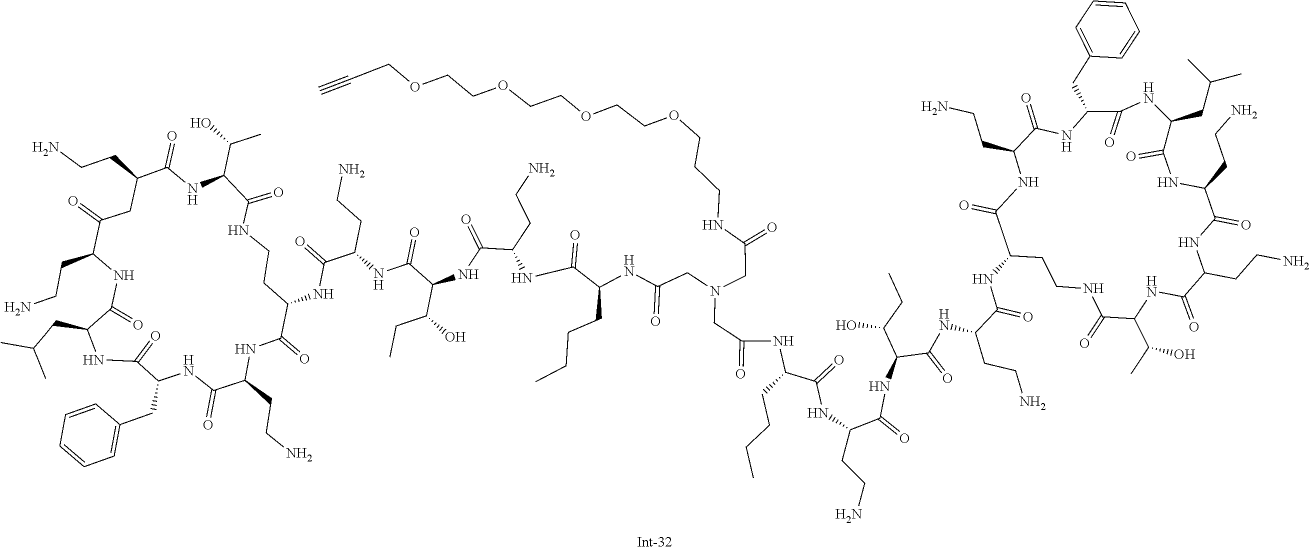 [SMILES: C#CCOCCOCCOCCOCCCNC(=O)CN(CC(=O)N[C@@H](CCCC)C(=O)N[C@@H](CCN)C(=O)N[C@H](C(=O)N[C@@H](CCN)C(=O)N[C@H]1CCCNC(=O)[C@H]([C@@H](C)O)NC(=O)[C@H](CCN)CC(=O)C[C@H](CCN)NC(=O)[C@H](CCC(C)C)NC(=O)[C@@H](Cc2ccccc2)NC(=O)[C@H](CCN)NC1=O)[C@H](O)CC)CC(=O)N[C@@H](CCCC)C(=O)N[C@@H](CCN)C(=O)N[C@H](C(=O)N[C@@H](CCN)C(=O)N[C@H]1CCNC(=O)C([C@@H](C)O)NC(=O)C(CCN)NC(=O)[C@H](CCN)NC(=O)[C@H](CC(C)C)NC(=O)[C@@H](Cc2ccccc2)NC(=O)[C@H](CCN)NC1=O)[C@H](O)CC]

Product from step m was treated in 2 mL DCM and 2 mL TFA at room temperature. The solution was stirred 10 min, then concentrated and purified by RPLC using an Isco CombiFlash liquid chromatograph eluted with 0% to 100% acetonitrile and water, using formic acid as the modifier. Yield of Int-32 was 0.120 g, 67% yield. Ion(s) found by LCMS: [M/4]+H+=691.4, [M/5]+H+=553.3, [M/6]+H+=461.3, [M/7]+H+=395.5.

### Example 68—Synthesis of Int-33

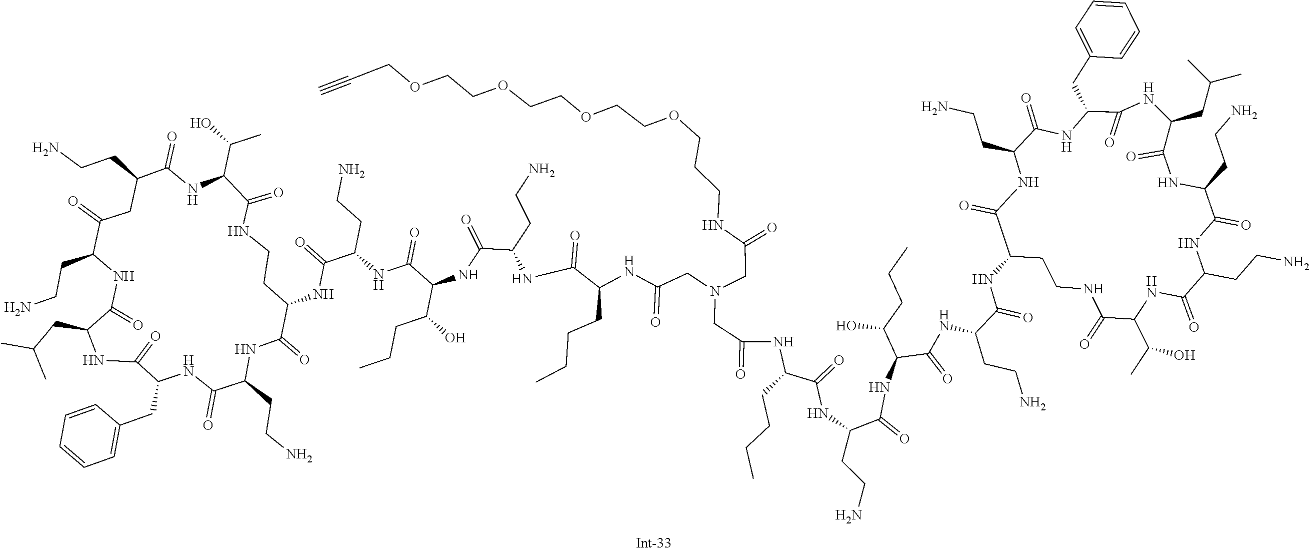 [SMILES: C#CCOCCOCCOCCOCCCNC(=O)CN(CC(=O)N[C@@H](CCCC)C(=O)N[C@@H](CCN)C(=O)N[C@H](C(=O)N[C@@H](CCN)C(=O)N[C@H]1CCCNC(=O)[C@H]([C@@H](C)O)NC(=O)[C@H](CCN)CC(=O)C[C@H](CCN)NC(=O)[C@H](CCC(C)C)NC(=O)[C@@H](Cc2ccccc2)NC(=O)[C@H](CCN)NC1=O)[C@H](O)CCC)CC(=O)N[C@@H](CCCC)C(=O)N[C@@H](CCN)C(=O)N[C@H](C(=O)N[C@@H](CCN)C(=O)N[C@H]1CCNC(=O)C([C@@H](C)O)NC(=O)C(CCN)NC(=O)[C@H](CCN)NC(=O)[C@H](CC(C)C)NC(=O)[C@@H](Cc2ccccc2)NC(=O)[C@H](CCN)NC1=O)[C@H](O)CCC]

The title compound Int-33 was prepared analogously to Example 67, where propionaldehyde was substituted with butyraldehyde in step a of the sequence. Ion(s) found by LCMS: [M/4]+H+=698.4, [M/5]+H+=558.9, [M/6]+H+=465.9, [M/7]+H+=399.5.

### Example 69—Synthesis of Int-34

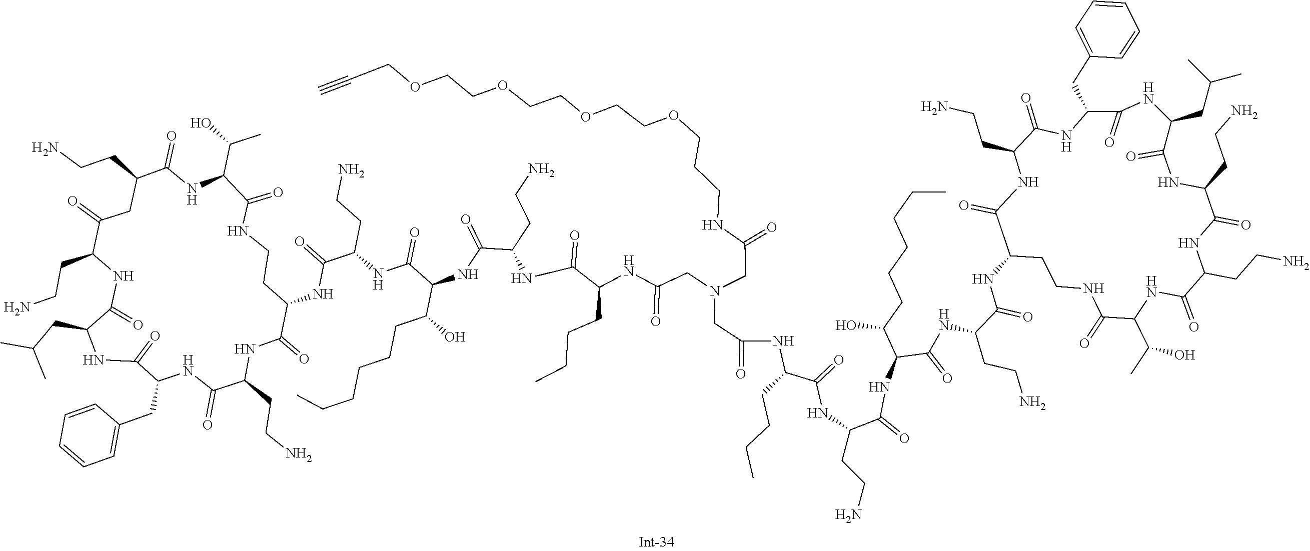 [SMILES: C#CCOCCOCCOCCOCCCNC(=O)CN(CC(=O)N[C@@H](CCCC)C(=O)N[C@@H](CCN)C(=O)N[C@H](C(=O)N[C@@H](CCN)C(=O)N[C@H]1CCCNC(=O)[C@H]([C@@H](C)O)NC(=O)[C@H](CCN)CC(=O)C[C@H](CCN)NC(=O)[C@H](CCC(C)C)NC(=O)[C@@H](Cc2ccccc2)NC(=O)[C@H](CCN)NC1=O)[C@H](O)CCCCCC)CC(=O)N[C@@H](CCCC)C(=O)N[C@@H](CCN)C(=O)N[C@H](C(=O)N[C@@H](CCN)C(=O)N[C@H]1CCNC(=O)C([C@@H](C)O)NC(=O)C(CCN)NC(=O)[C@H](CCN)NC(=O)[C@H](CC(C)C)NC(=O)[C@@H](Cc2ccccc2)NC(=O)[C@H](CCN)NC1=O)[C@H](O)CCCCCC]

The title compound Int-34 was prepared analogously to Example 67, where propionaldehyde was substituted with heptanal in step a of the sequence. Ion(s) found by LCMS: [M/4]+H+=719.4.4, [M/5]+H+=575.7, [M/6]+H+=479.9, [M/7]+H+=411.5.

### Example 70—Synthesis of Int-35

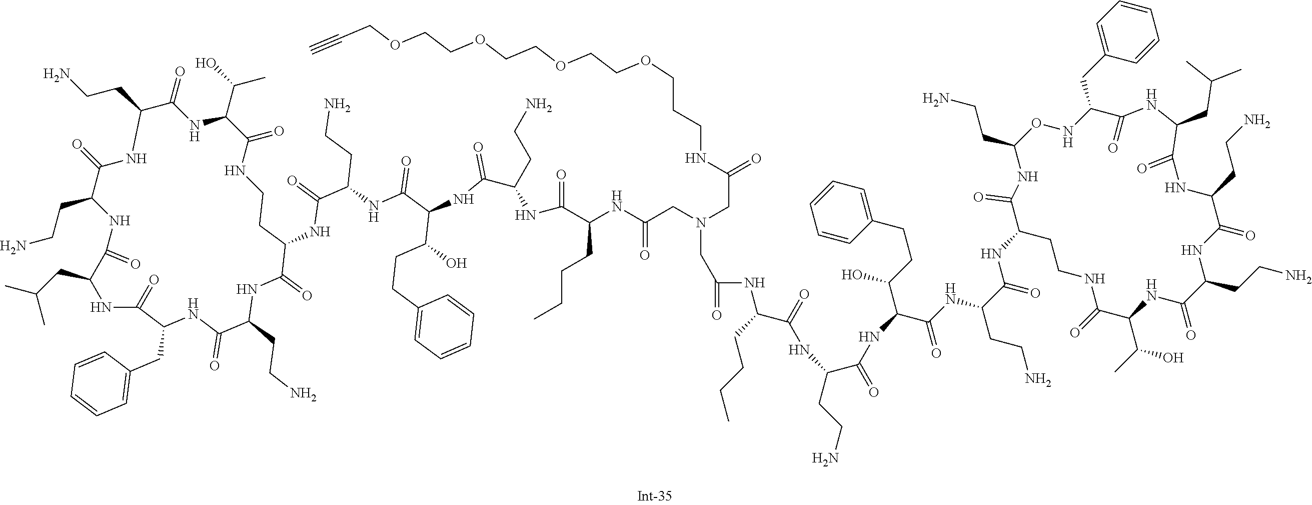 [SMILES: C#CCOCCOCCOCCOCCCNC(=O)CN(CC(=O)N[C@@H](CCCC)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NO[C@H](CCN)NC1=O)[C@H](O)CCc1ccccc1)CC(=O)N[C@@H](CCCC)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCc1ccccc1]

The title compound Int-35 was prepared analogously to Example 67, where propionaldehyde was substituted with 3-phenylpropionaldehyde in step a of the sequence. Ion(s) found by LCMS: [M/4]+H+=729.4, [M/5]+H+=582.7, [M/6]+H+=486.6, [M/7]+H+=417.2.

### Example 71—Synthesis of Int-36

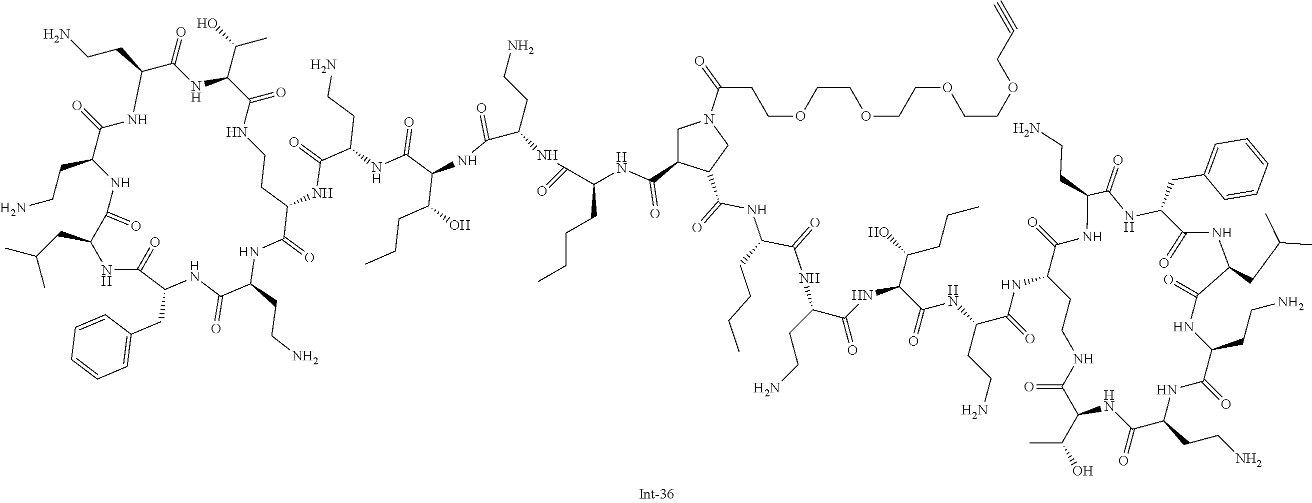

The title compound Int-36 was prepared analogously to Example 68, where the di-acid intermediate was substituted with the di-acid described in Example 41, step m. Ion(s) found by LCMS: [M/4]+H+=693.2, [M/5]+H+=554.5, [M/6]+H+=463.1, [M/7]+H+=397.1.

### Example 72—Synthesis of Int-37

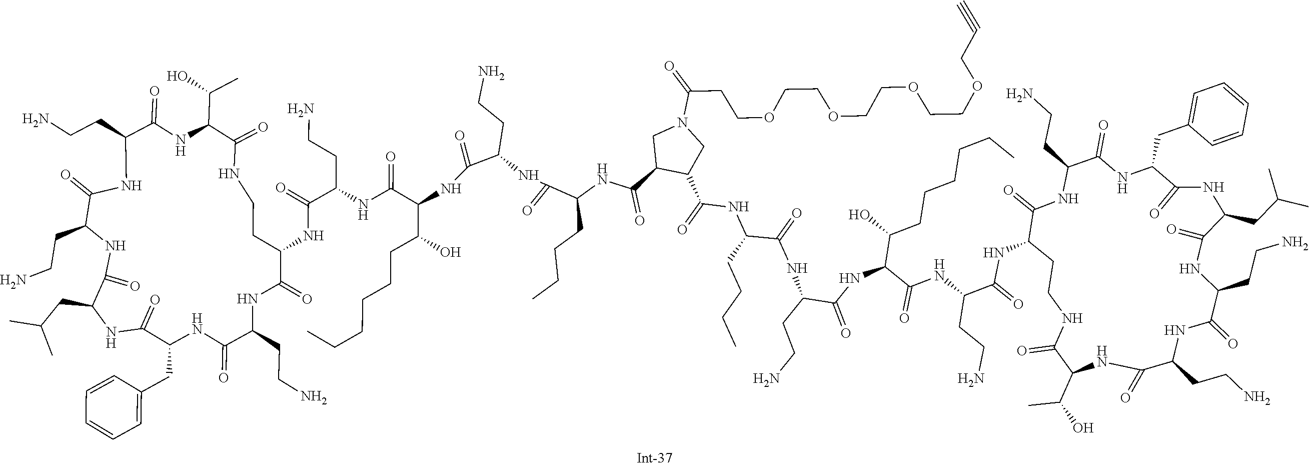

The title compound Int-37 was prepared analogously to Example 69, where the di-acid intermediate was substituted with di-acid described in Example 41, step m. Ion(s) found by LCMS: [M/4]+H+=714.2, [M/5]+H+=572.3, [M/6]+H+=477.1, [M/7]+H+=409.1.

### Example 73—Synthesis of Int-38

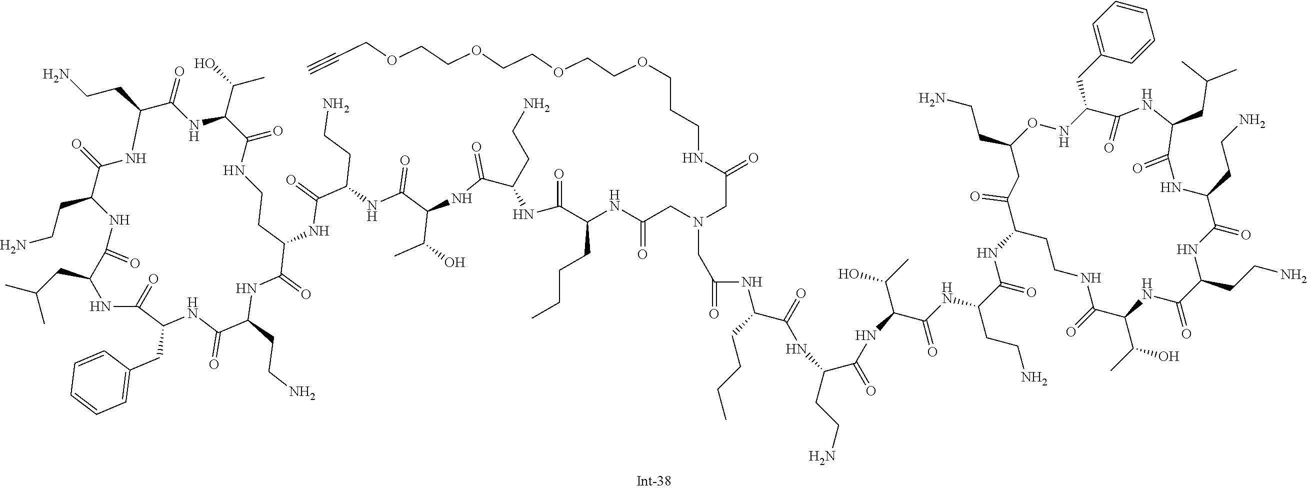 [SMILES: C#CCOCCOCCOCCOCCCNC(=O)CN(CC(=O)N[C@@H](CCCC)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NO[C@H](CCN)CC1=O)[C@@H](C)O)CC(=O)N[C@@H](CCCC)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O]

The title compound Int-38 was prepared analogously to Example 67, where penta-Boc-polymyxin B decapeptide containing an extended threonine was substituted with the Int-7a (described in Example 8). Ion(s) found by LCMS: [M/4]+H+=684.4. [M/5]+H+=547.7, [M/6]+H+=456.6.

### Example 74—Synthesis of Int-39

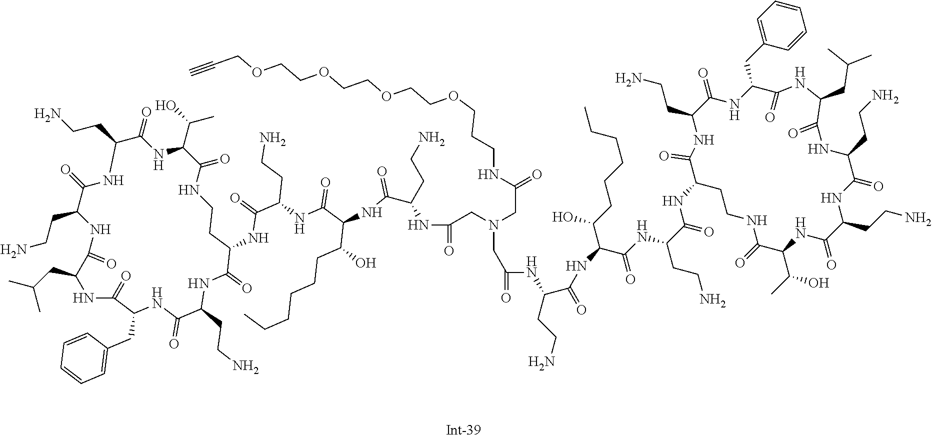 [SMILES: C#CCOCCOCCOCCOCCCNC(=O)CN(CC(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCCCCC)CC(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCCCCC]

The title compound Int-39 was prepared analogously to Example 69, where the di-acid intermediate was replaced with the di-acid intermediate described in step a of Example 90. Ion(s) found by LCMS: [M/4]+H+=662.9, [M/5]+H+=530.5, [M/6]+H+=442.3, [M/7]+H+=379.2.

### Example 75—Synthesis of Int-40

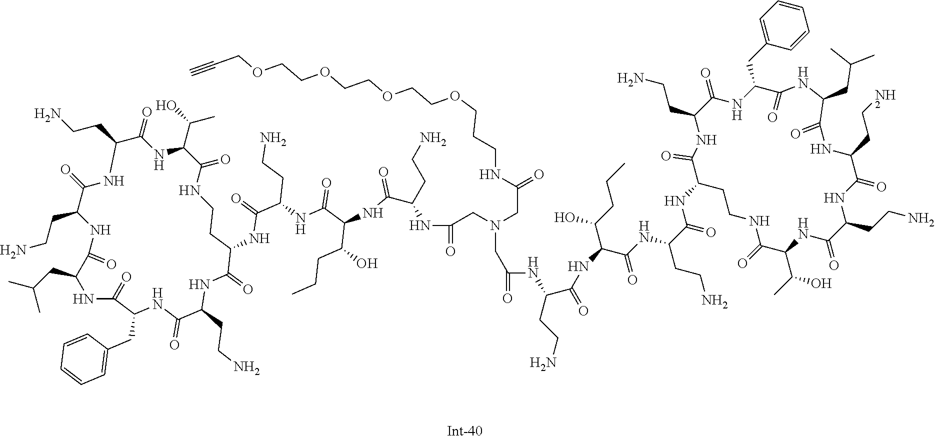 [SMILES: C#CCOCCOCCOCCOCCCNC(=O)CN(CC(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCC)CC(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCC]

The title compound Int-40 was prepared analogously to Example 68, where the di-acid intermediate was replaced with the di-acid intermediate described in step a of Example 90. Ion(s) found by LCMS: [M/4]+H+=641.9, [M/5]+H+=513.7, [M/6]+H+=428.3, [M/7]+H+=367.2.

### Example 76—Synthesis of Int-41

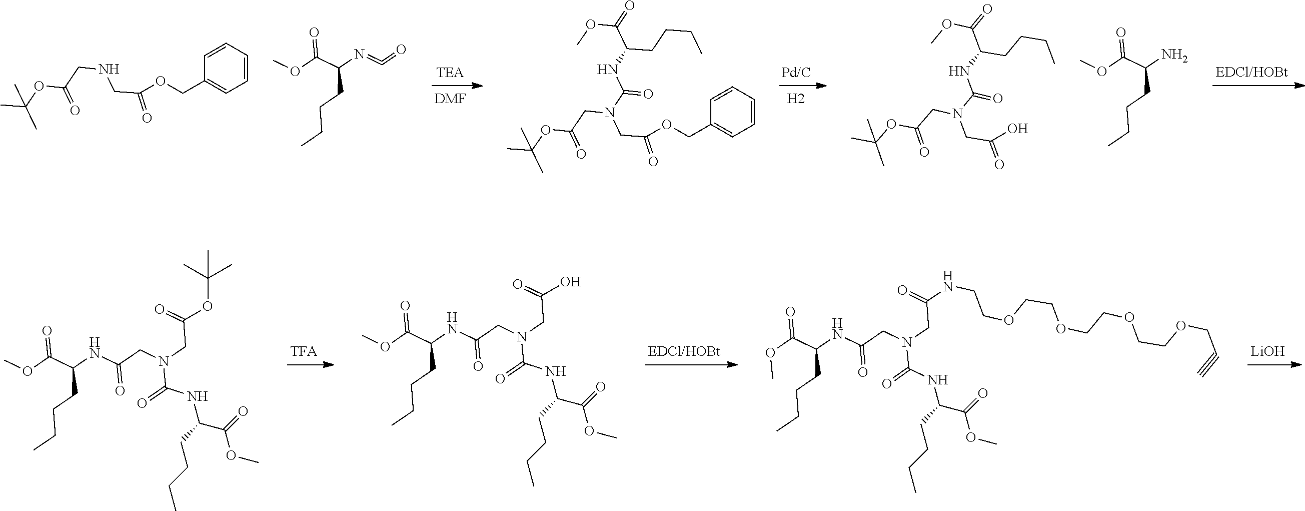

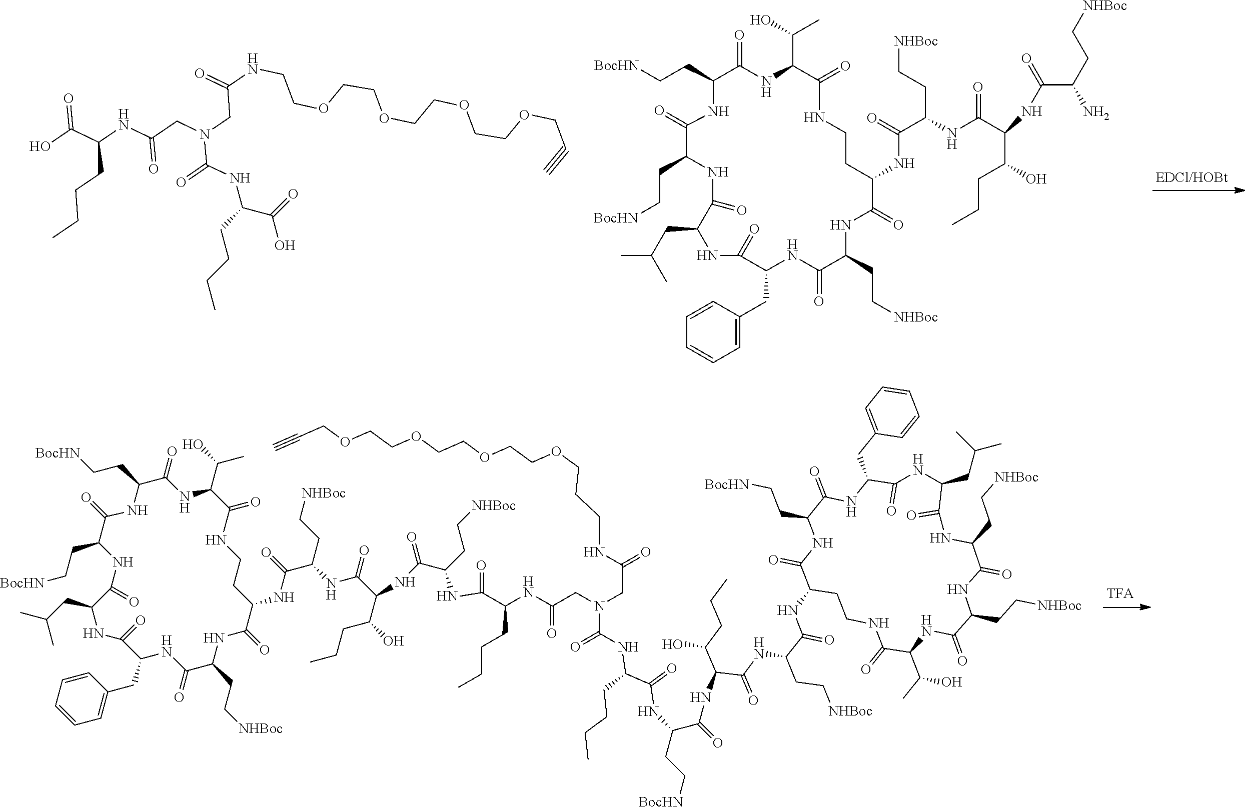

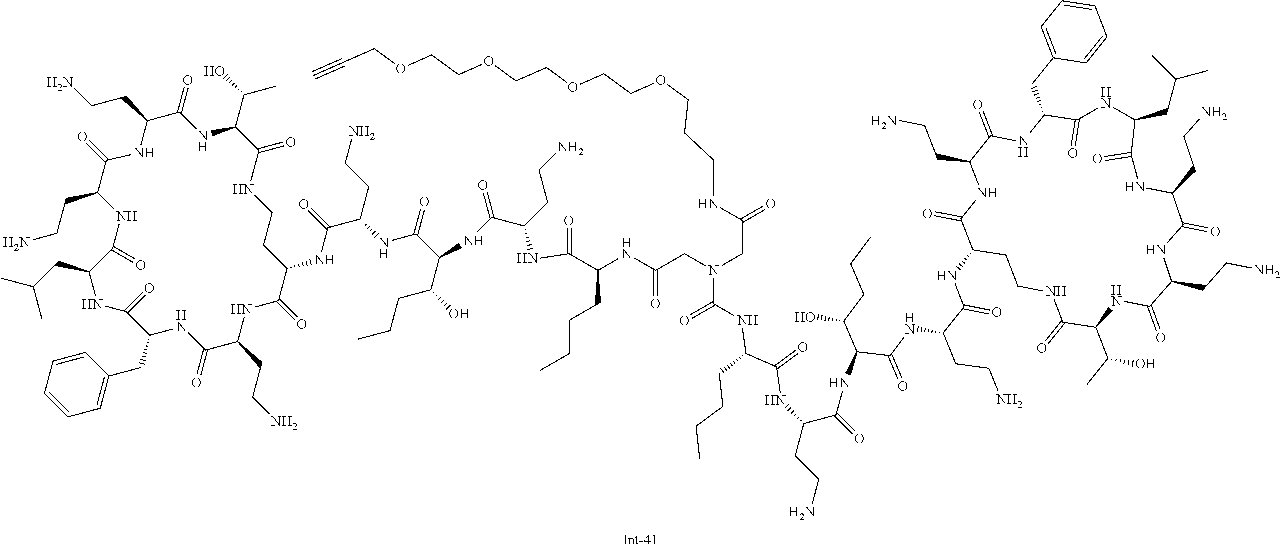 [SMILES: C#CCOCCOCCOCCOCCCNC(=O)CN(CC(=O)N[C@@H](CCCC)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCC)C(=O)N[C@@H](CCCC)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCC]

### Step a

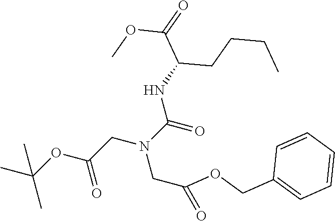 [SMILES: CCCC[C@H](NC(=O)N(CC(=O)OCc1ccccc1)CC(=O)OC(C)(C)C)C(=O)OC]

Methyl N-(oxomethylidene)-L-norleucinate (510 mg, 3 mmol) was added into a solution of benzyl tert-butyl 2,2′-azanediyldiacetate (830 mg, 3 mmol) and triethyl amine (0.52 mL, 3 mmol) in 5 mL DMF at room temperature, then stirred overnight. The reaction was concentrated then purified by RPLC using an Isco CombiFlash liquid chromatograph eluted with 5% to 100% acetonitrile and water, using 0.1% TFA as the modifier. Yield of the products as an oil, 920 mg, 69% yield. Ion(s) found by LCMS: M+H=452.3.

### Step b

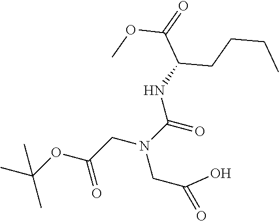 [SMILES: CCCC[C@H](NC(=O)N(CC(=O)O)CC(=O)OC(C)(C)C)C(=O)OC]

Product from the previous step (450 mg, 1 mmol) was dissolved into 10 mL methanol containing 5% water, then treated with 200 mg of 5% Pd/C and stirred at room temperature under a hydrogen atmosphere for 2 hours. The palladium charcoal was removed by filtration, and the filtrate was concentrated and used in next step without further purification.

### Step c

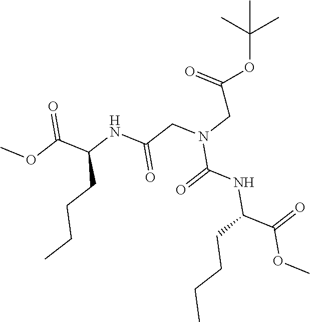

The compound from step b (450 mg, 1 mmol), methyl L-norleucinate (230 mg, 1 mmol) in DMF (5 mL) was added EDC (300 mg, 1.5 mmol), HOBT (230 mg, 1.5 mmol), DIEA (0.3 mL, 1.5 mmol) at room temperature. The solution was stirred overnight. The resulting solution was concentrated and purified by RPLC using an Isco CombiFlash liquid chromatograph eluted with 5% to 100% acetonitrile and water, using 0.1% TFA as the modifier. Yield of the product as an oil, 420 mg, 86% yield. Ion(s) found by LCMS: (M+H)+=488.3

### Step d

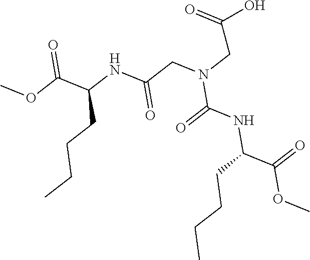

The compound from step c (500 mg, 1 mmol) was treated with 5 mL TFA at room temperature, after the completion the reaction, the solution was concentrated and used in next step without further purification.

### Step e

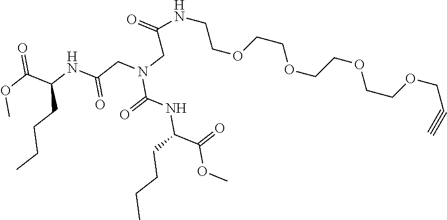 [SMILES: C#CCOCCOCCOCCOCCNC(=O)CN(CC(=O)N[C@@H](CCCC)C(=O)OC)C(=O)N[C@@H](CCCC)C(=O)OC]

The compound from step d (430 mg, 1 mmol), propargyl-PEG-4-NH2 (230 mg, 1 mmol) in DMF (5 mL) was added EDC (300 mg, 1.5 mmol), HOBT (230 mg, 1.5 mmol), DIEA (0.3 mL, 1.5 mmol) at room temperature. The solution was stirred overnight. The resulting solution was concentrated and purified by RPLC using an Isco CombiFlash liquid chromatograph eluted with 5% to 100% acetonitrile and water, using 0.1% TFA as the modifier. Yield of the product as an oil, 470 mg, 73% yield. Ion(s) found by LCMS: (M+H)+=645.4.

### Step f

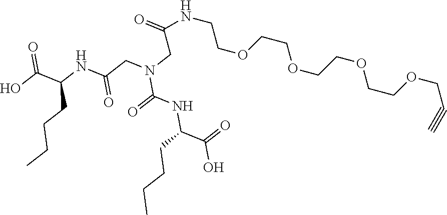 [SMILES: C#CCOCCOCCOCCOCCNC(=O)CN(CC(=O)N[C@@H](CCCC)C(=O)O)C(=O)N[C@@H](CCCC)C(=O)O]

Lithium hydroxide (24 mg, 1 mmol) in 1 mL water was added into a solution of the compound from step e (70 mg, 1 mmol) in 1 mL methanol and 1 mL THF. The reaction was monitored by LCMS. After completion, the solution was adjusted to pH 1 with Amberlite® IRN-77, then the resulting solution was filtered and concentrated and used in next step without further purification. Ion(s) found by LCMS: (M+H)+=616.4.

### Step g

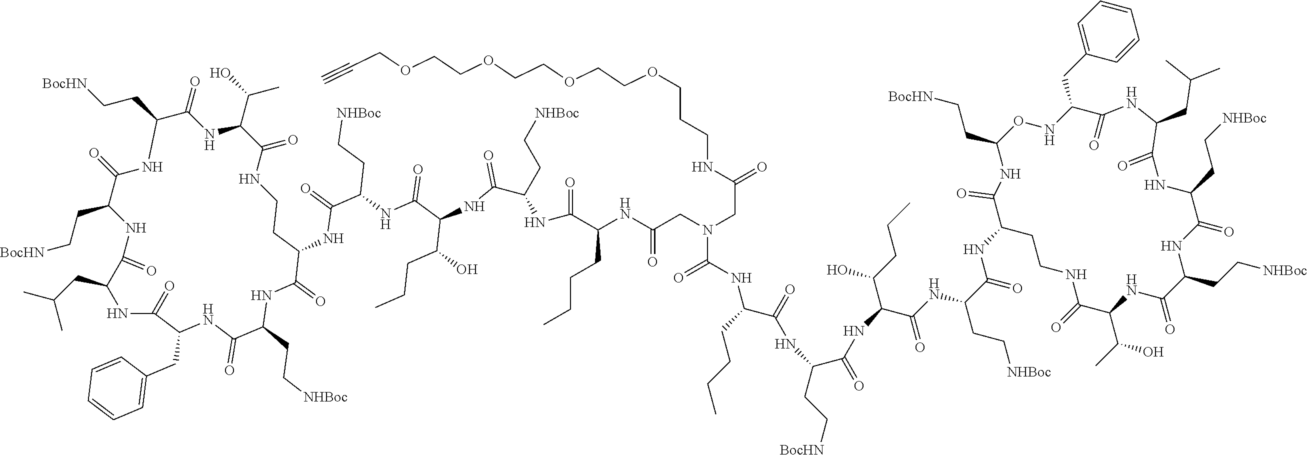

The compound from step f (62 mg, 0.1 mmol), and penta-Boc-polymyxin B decapeptide (320 mg, 0.2 mmol, 2 eq) in DMF (5 mL) were added EDC (60 mg, 0.3 mmol), HOBT (45 mg, 0.3 mmol), DIEA (0.14 mL, 1.0 mmol) at room temperature. The solution was stirred overnight. The resulting solution was concentrated and re-dissolved into 2 mL methanol, the resulting solution was added drop wise into 50 mL vigorously stirred water, this heterogeneous solution was stirred for 1 hour, then filtered with Buchner funnel with Whatman filter paper. The crude product was used for next step without purification.

### Step h

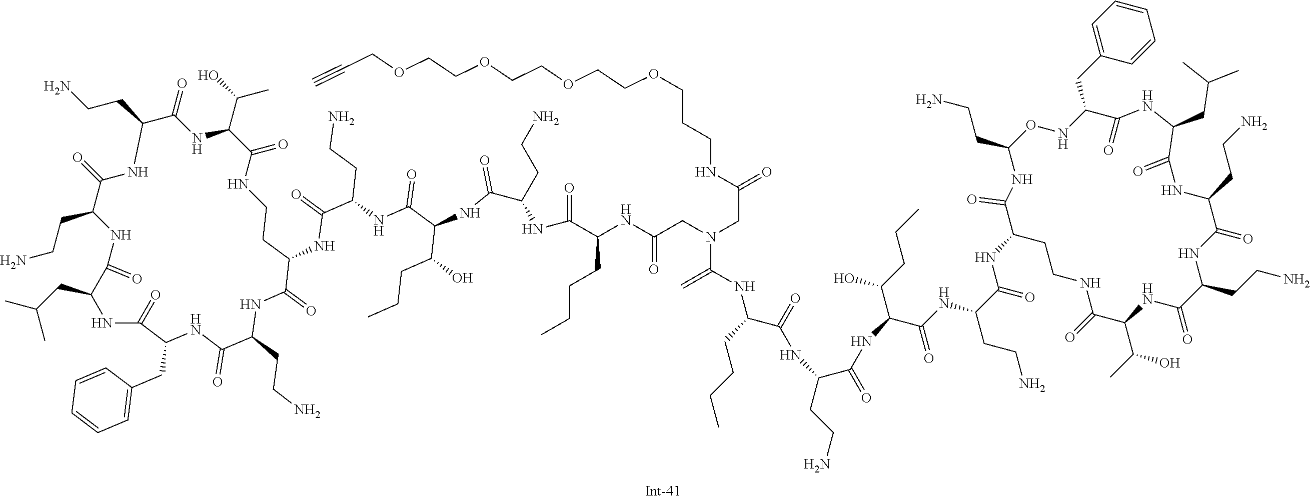 [SMILES: C#CCOCCOCCOCCOCCCNC(=O)CN(CC(=O)N[C@@H](CCCC)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCC)C(=C)N[C@@H](CCCC)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NO[C@H](CCN)NC1=O)[C@H](O)CCC]

The compound from step g was treated in 2 mL DCM and 2 mL TFA at room temperature, the solution was stirred 10 min, then concentrated and purified by RPLC using an Isco CombiFlash liquid chromatograph eluted with 0% to 100% acetonitrile and water, using formic acid as the modifier. Yield of Int-41 was 0.070 g, 26% yield. Ion(s) found by LCMS: [M/4]+H+=694.9, [M/5]+H+=555.1, [M/6]+H+=463.6, [M/7]+H+=397.5.

### Example 77—Synthesis of Int-42

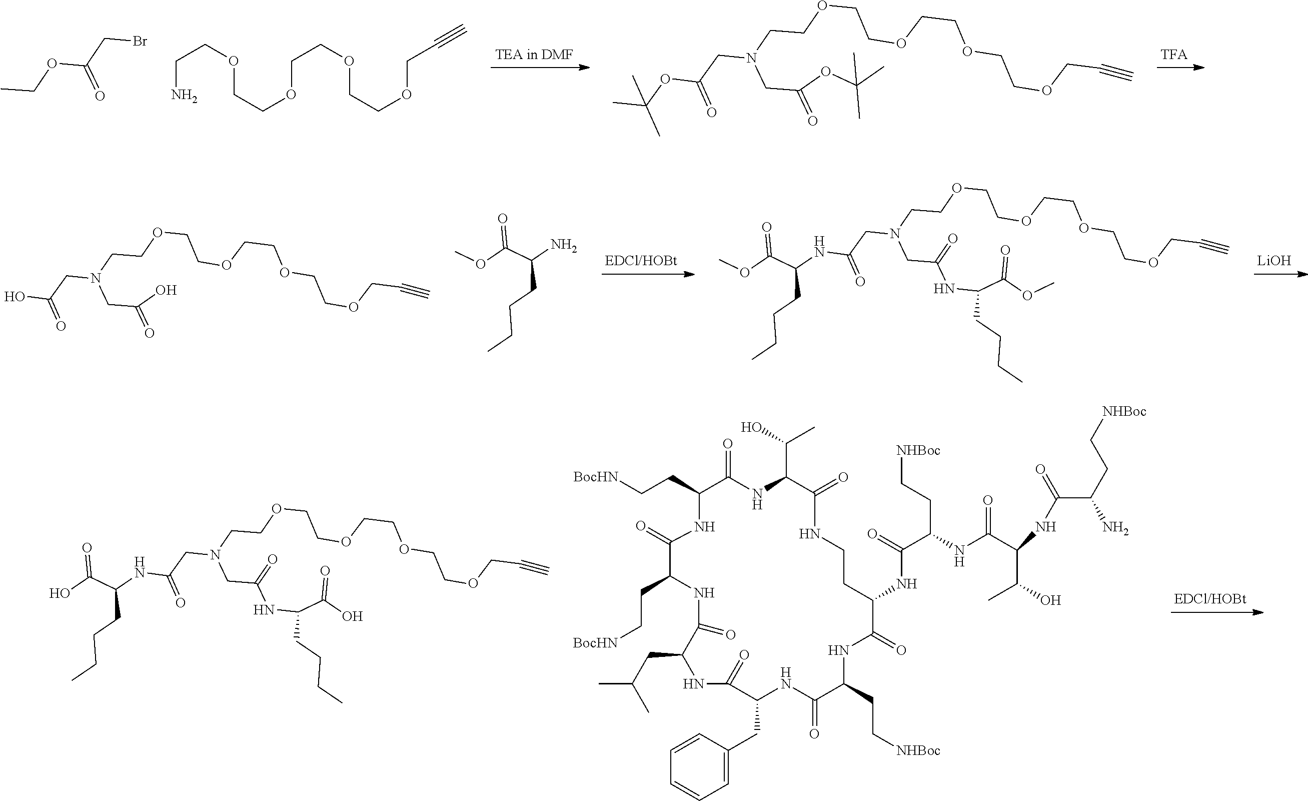

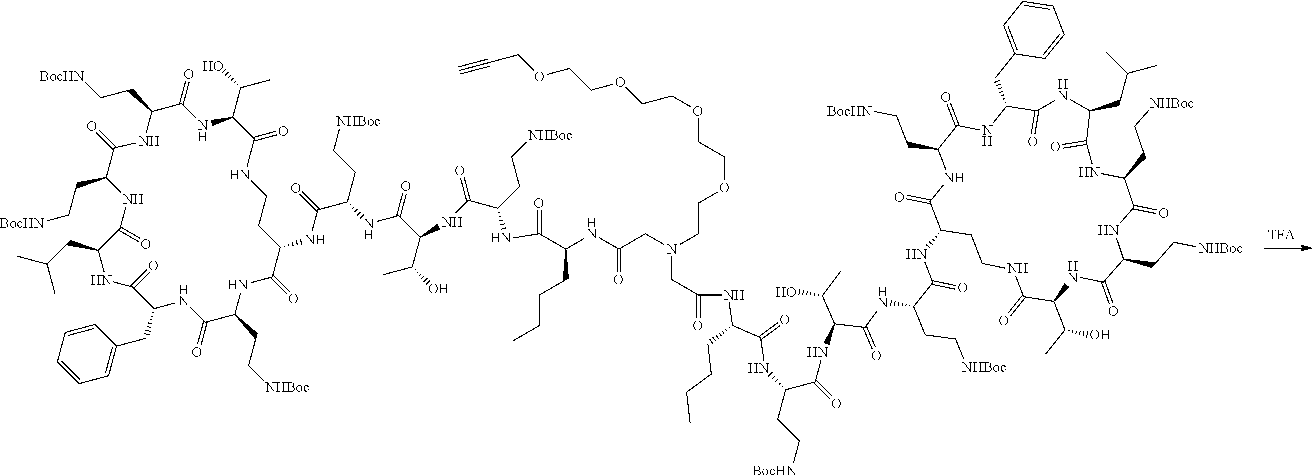

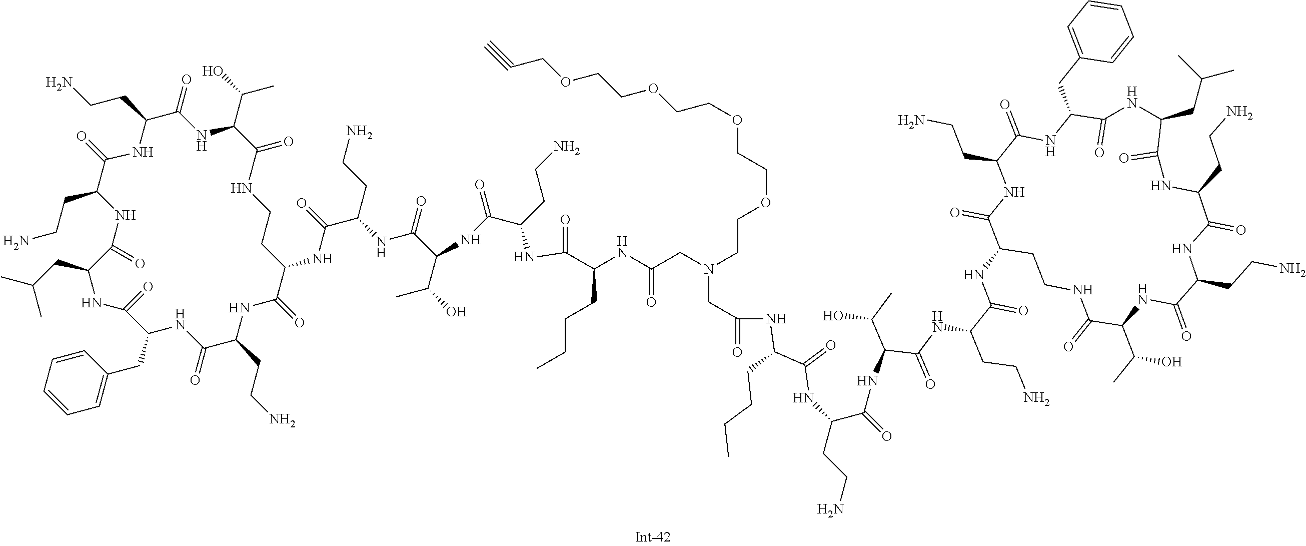 [SMILES: C#CCOCCOCCOCCOCCN(CC(=O)N[C@@H](CCCC)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O)CC(=O)N[C@@H](CCCC)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O]

### Step a

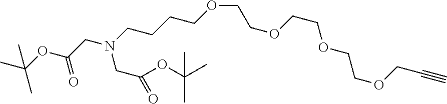 [SMILES: C#CCOCCOCCOCCOCCCCN(CC(=O)OC(C)(C)C)CC(=O)OC(C)(C)C]

t-butyl Bromo-acetate (1.2 mg, 6 mmol) was added into a solution of propargyl-PEG-4-NH2 (460 mg, 2 mmol) and DIPEA (1.4 mL, 10 mmol) in 20 mL DMF, The resulting solution was stirred at room temperature for overnight. The reaction solution was concentrated and purified by flash chromatography to provide products. Yield of product AS AN OIL, 700 mg, 76%. Ion(s) found by LC/MS [M+H]+=460.3.

### Step b

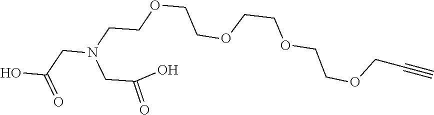 [SMILES: C#CCOCCOCCOCCOCCN(CC(=O)O)CC(=O)O]

tert-butyl 3-(2-tert-butoxy-2-oxoethyl)-6,9,12,15-tetraoxa-3-azaoctadec-17-yn-1-oate (230 mg, 0.5 mmol) was treated with 5 mL TFA at room temperature, after the completion the reaction, the solution was concentrated and used in next step without further purification.

### Step c

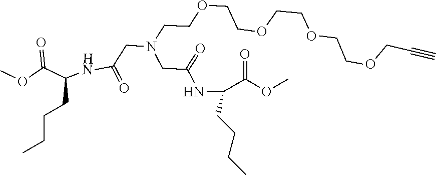 [SMILES: C#CCOCCOCCOCCOCCN(CC(=O)N[C@@H](CCCC)C(=O)OC)CC(=O)N[C@@H](CCCC)C(=O)OC]

The compound from step b (700 mg, 2 mmol), methyl L-norleucinate (630 mg, 4.2 mmol) in DMF (40 mL) was added EDC (1.0 g, 5 mmol), HOBT (0.9 g, 6 mmol), DIEA (1.0 mL, 7 mmol) at room temperature. The solution was stirred overnight. The resulting solution was concentrated and purified by RPLC using an Isco CombiFlash liquid chromatograph eluted with 5% to 100% acetonitrile and water, using 0.1% TFA as the modifier. Yield of product as an oil, 910 mg, 75%. Ion(s) found by LCMS: (M+H)+=602.4.

### Step d

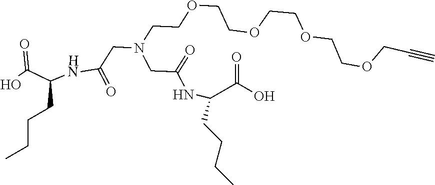 [SMILES: C#CCOCCOCCOCCOCCN(CC(=O)N[C@@H](CCCC)C(=O)O)CC(=O)N[C@@H](CCCC)C(=O)O]

Lithium hydroxide 24 mg, 1 mmol) in 2 mL water was added into a solution of the amino acid ester from step c (0.06 g, 0.1 mmol) in 2 mL methanol and 2 mL THF. The reaction was monitored by LCMS. After completion, the solution was adjusted to pH 1 with Amberlite® IRN-77, then the resulting solution was filtered and concentrated and used in next step without further purification. Ion(s) found by LCMS: (M+H)+=574.4.

### Step e

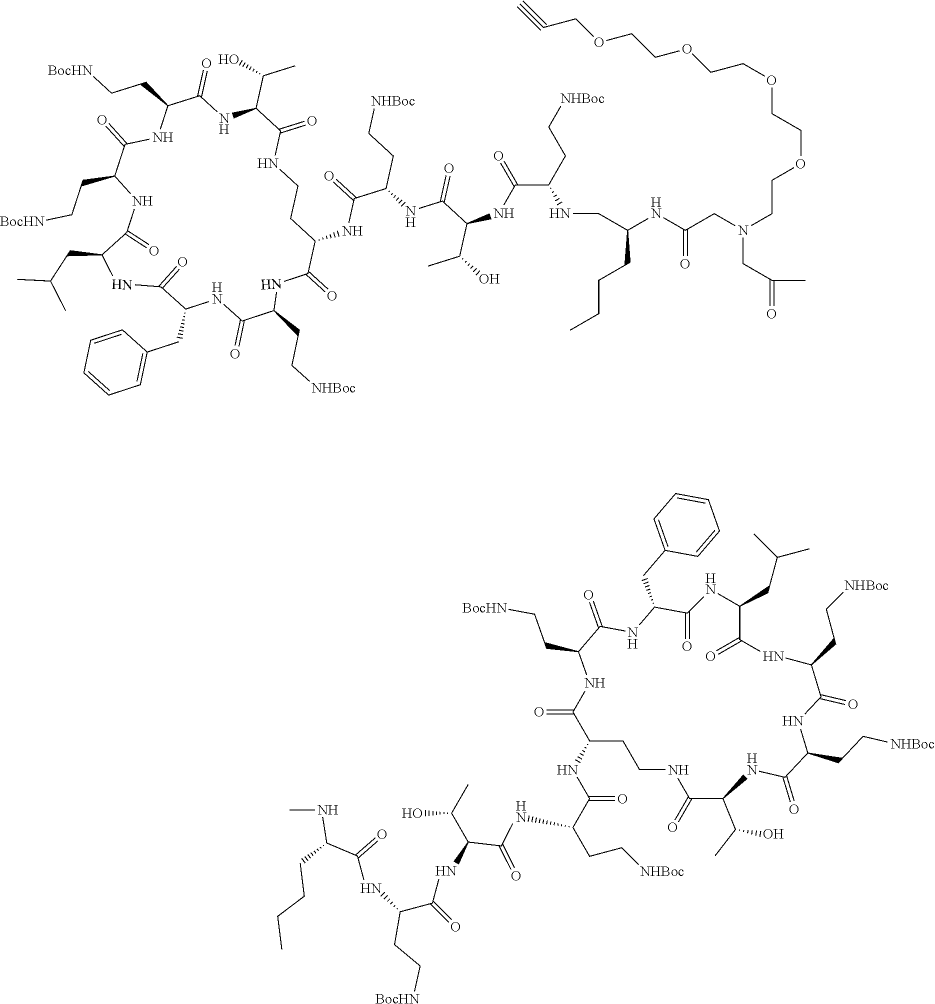

The compound from step d (57 mg, 0.1 mmol), and penta-Boc-polymyxin B decapeptide (350 mg, 0.22 mmol, 2.1 eq) in DMF (5 mL) were added EDC (60 mg, 0.3 mmol), HOBT (45 mg, 0.3 mmol), DIEA (0.14 mL, 1.0 mmol) at room temperature. The solution was stirred overnight. The resulting solution was concentrated and re-dissolved into 2 mL methanol, the resulting solution was added drop wise into 50 mL vigorously stirred water, this heterogeneous solution was stirred for 1 hour, then filtered with Buchner funnel with Whatman filter paper. The crude product was used for next step without purification.

### Step f

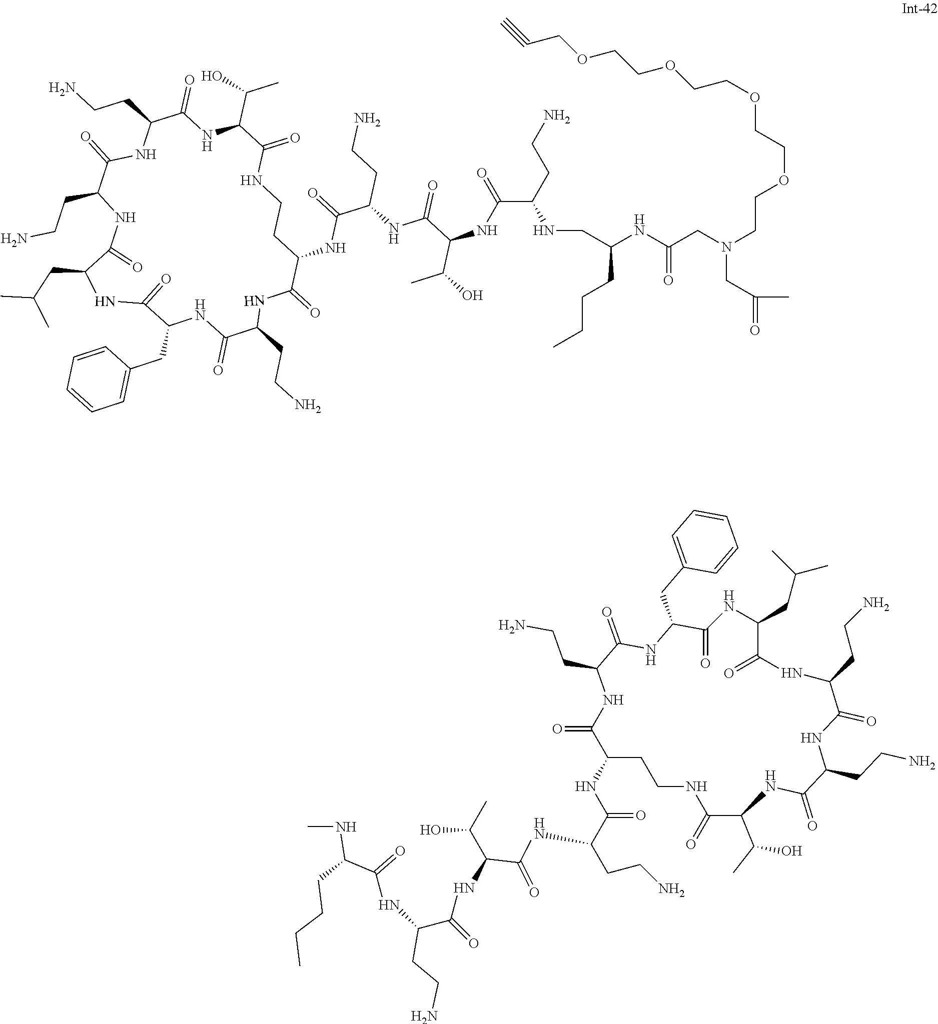 [SMILES: C#CCOCCOCCOCCOCCN(CC(C)=O)CC(=O)N[C@@H](CCCC)CN[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O ; CCCC[C@H](NC)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O]

Product from step e was treated in 2 mL DCM and 2 mL TFA at room temperature, the solution was stirred 10 min, then concentrated and purified by RPLC using an Isco CombiFlash liquid chromatograph eluted with 0% to 100% acetonitrile and water, using formic acid as the modifier. Yield of Int-42 was 0.120 g, 67% yield. Ion(s) found by LCMS: [M/4]+H+=666.6, [M/5]+H+=533.5, [M/6]+H+=444.7. [M/7]+H+=381.4.

### Example 78—Synthesis of Int-43

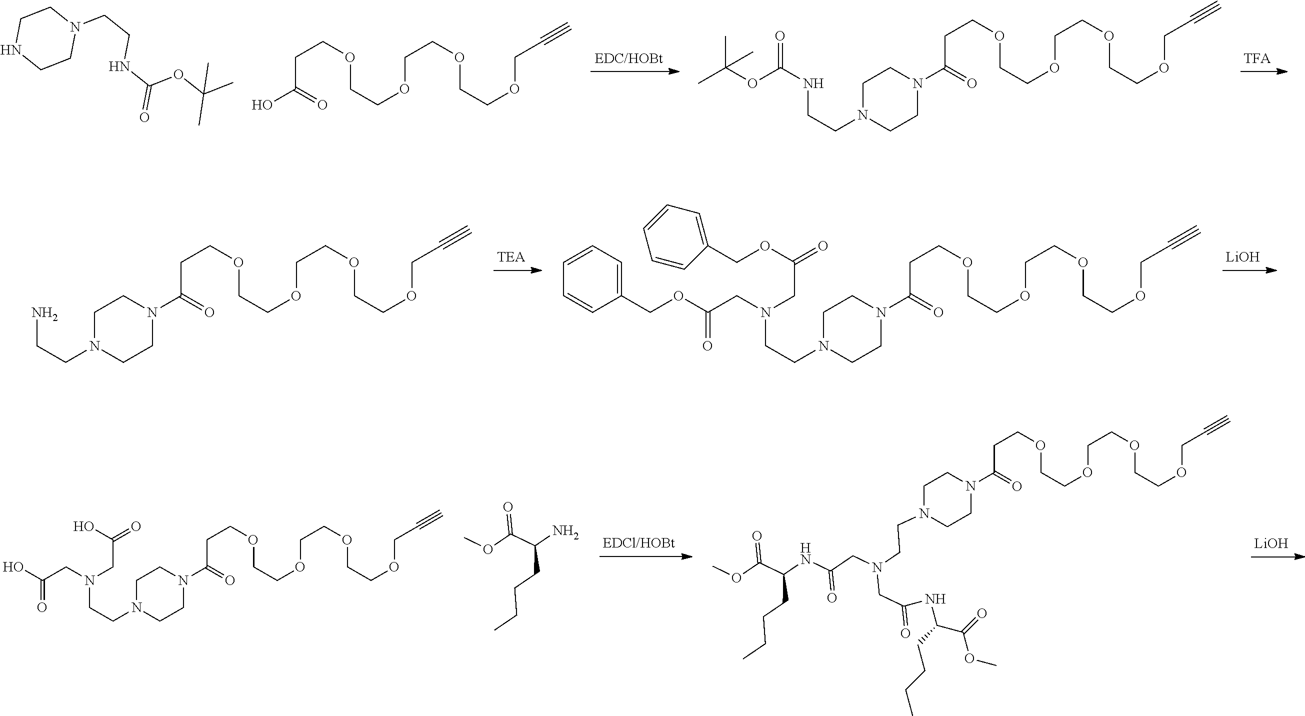

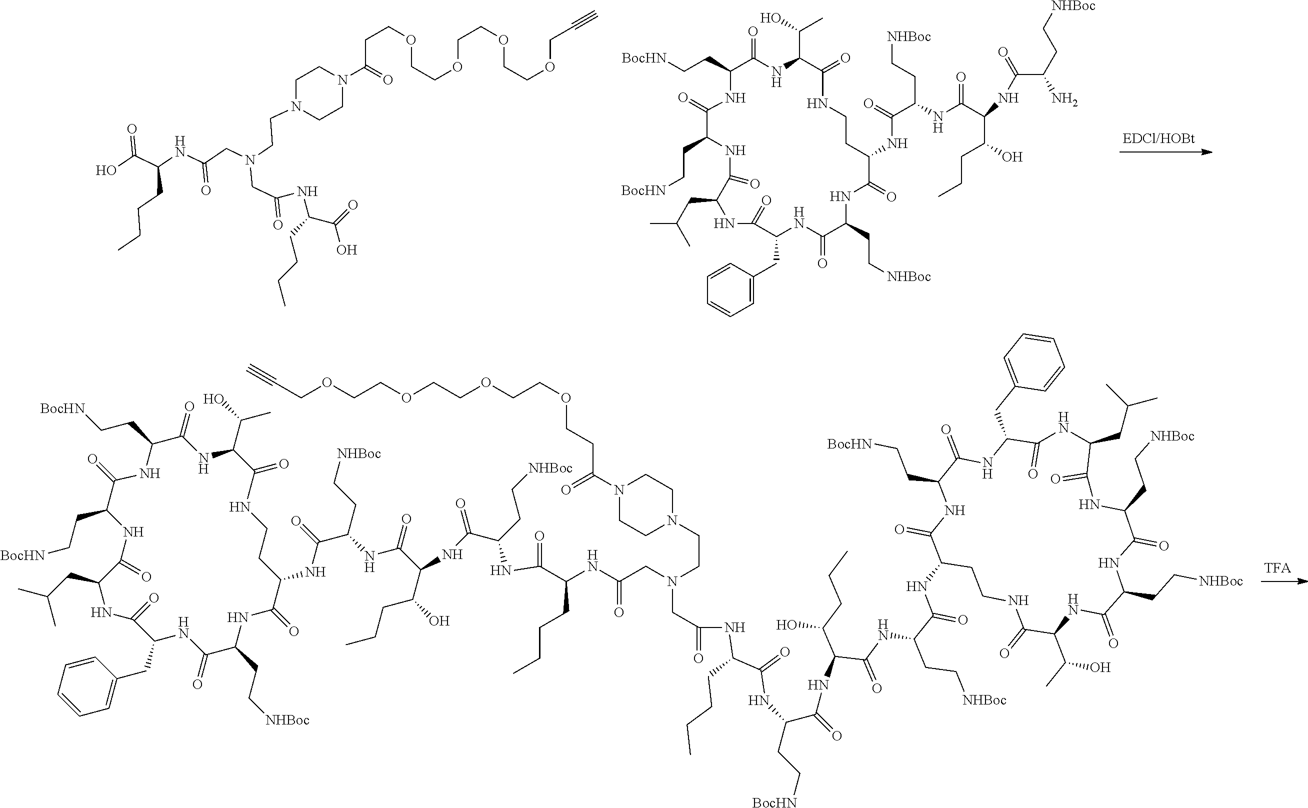

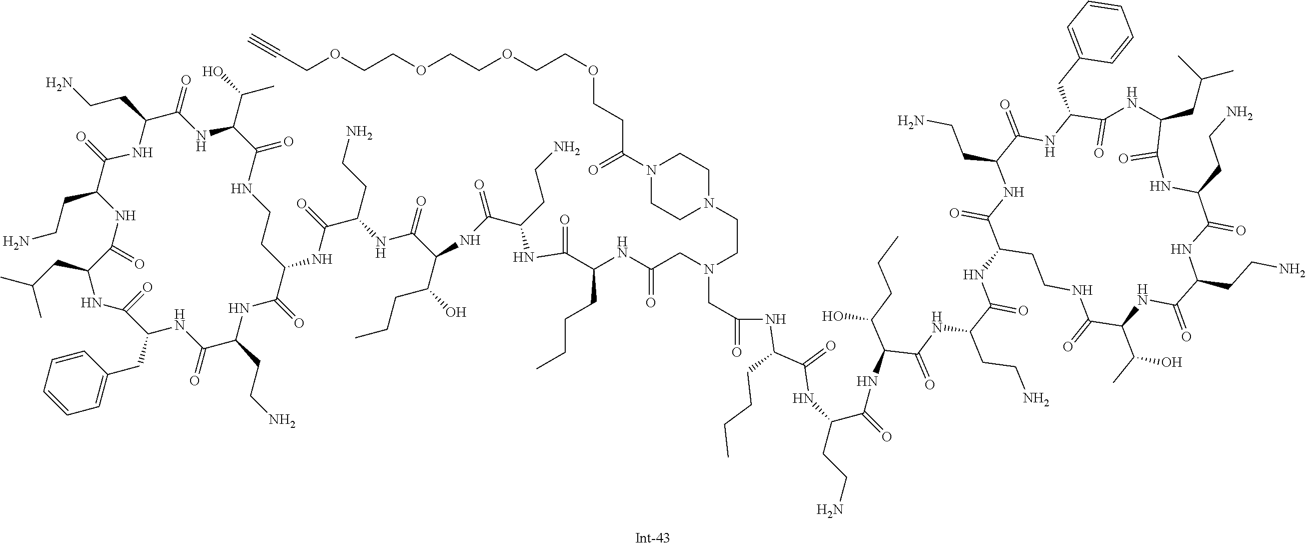 [SMILES: C#CCOCCOCCOCCOCCC(=O)N1CCN(CCN(CC(=O)N[C@@H](CCCC)C(=O)N[C@@H](CCN)C(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@H](O)CCC)CC(=O)N[C@@H](CCCC)C(=O)N[C@@H](CCN)C(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@H](O)CCC)CC1]

### Step a

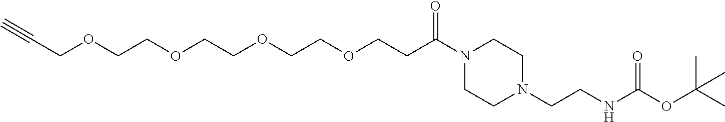 [SMILES: C#CCOCCOCCOCCOCCC(=O)N1CCN(CCNC(=O)OC(C)(C)C)CC1]

Propargyl-PEG4-acid (850 mg, 3.2 mmol), tert-butyl [2-(piperazin-1-yl)ethyl]carbamate (750 mg, 3.2 mmol) in DMF (20 mL) was added EDC (1.0 g, 5 mmol), HOBT (0.9 g, 6 mmol), DIEA (1.0 mL, 7 mmol) at room temperature. The solution was stirred overnight. The resulting solution was concentrated and purified by RPLC using an Isco CombiFlash liquid chromatograph eluted with 5% to 100% acetonitrile and water, using 0.1% TFA as the modifier. Yield of product as an oil, 850 mg, 56%. Ion(s) found by LCMS: (M+H)⁺=472.3.

### Step b

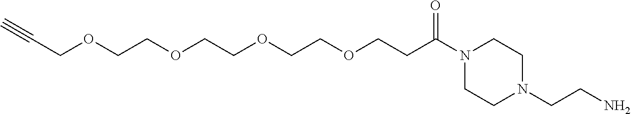 [SMILES: C#CCOCCOCCOCCOCCC(=O)N1CCN(CCN)CC1]

Intermediate from the previous step (470 mg, 1 mmol) was treated with 5 mL TFA at room temperature. After completion of the reaction, the solution was concentrated and used in next step without further purification.

### Step c

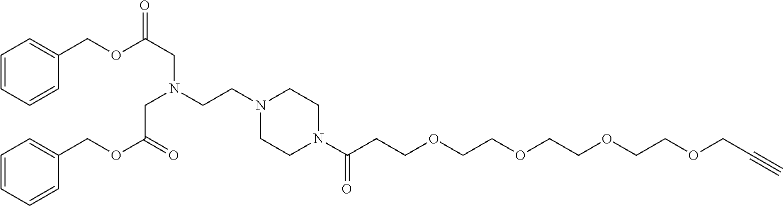 [SMILES: C#CCOCCOCCOCCOCCC(=O)N1CCN(CCN(CC(=O)OCc2ccccc2)CC(=O)OCc2ccccc2)CC1]

Benzyl Bromo-acetate (0.8 mg, 3.5 mmol) was added into a solution of compounds from step b (470 mg, 1 mmol, TFA salt) and DIPEA (1.0 mL, 7 mmol) in 10 mL DMF, The resulting solution was stirred at room temperature for overnight. The reaction solution was concentrated and purified by flash chromatography to provide products. Yield of product as an oil, 620 mg, 92%. Ion(s) found by LC/MS [M+H]⁺=668.3.

### Step d

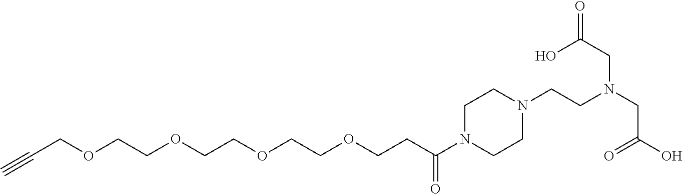 [SMILES: C#CCOCCOCCOCCOCCC(=O)N1CCN(CCN(CC(=O)O)CC(=O)O)CC1]

Lithium hydroxide (120 mg, 5 mmol) in 2 mL water was added into a solution of the amino acid ester from step d (0.500 g, 0.74 mmol) in 2 mL methanol and 2 mL THF. The reaction was monitored by LCMS. After completion, the solution was adjusted to pH 1 with Amberlite® IRN-77, then the resulting solution was filtered and concentrated and used in next step without further purification. Yield of product as an oil 480 mg, 100% yield. Ion(s) found by LCMS: (M+H)⁺=488.3.

### Step e

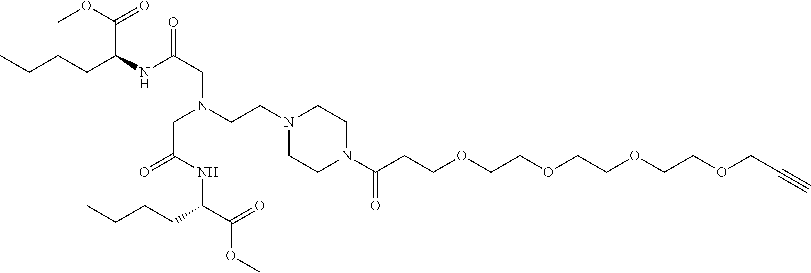 [SMILES: C#CCOCCOCCOCCOCCC(=O)N1CCN(CCN(CC(=O)N[C@@H](CCCC)C(=O)OC)CC(=O)N[C@@H](CCCC)C(=O)OC)CC1]

To a solution of intermediate from the previous step (360 mg, 0.73 mmol) in DMF (20 mL) was added EDC (300 mg, 1.5 mmol), HOBT (230 mg, 1.5 mmol), DIEA (0.28 mL, 2 mmol) at room temperature. The solution was stirred overnight. The resulting solution was concentrated and purified by RPLC using an Isco CombiFlash liquid chromatograph eluted with 5% to 100% acetonitrile and water, using 0.1% TFA as the modifier. Yield of product as an oil, 420 mg, 77%. Ion(s) found by LCMS: (M+H)⁺=742.5.

### Step f

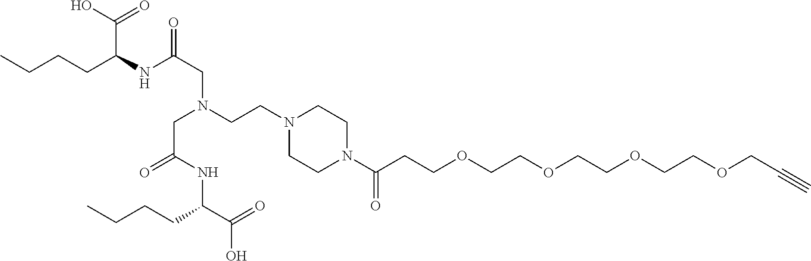 [SMILES: C#CCOCCOCCOCCOCCC(=O)N1CCN(CCN(CC(=O)N[C@@H](CCCC)C(=O)O)CC(=O)N[C@@H](CCCC)C(=O)O)CC1]

Lithium hydroxide (24 mg, 1 mmol) in 2 mL water was added into a solution of the amino acid ester from step d (0.150 g, 0.2 mmol) in 2 mL methanol and 2 mL THF. The reaction was monitored by LCMS. After completion, the solution was adjusted to pH 1 with Amberlite® IRN-77, then the resulting solution was filtered and concentrated and used in next step without further purification. Yield of product as an oil, 140 mg, 100% yield. Ion(s) found by LCMS: (M+H)⁺=714.4.

### Step g

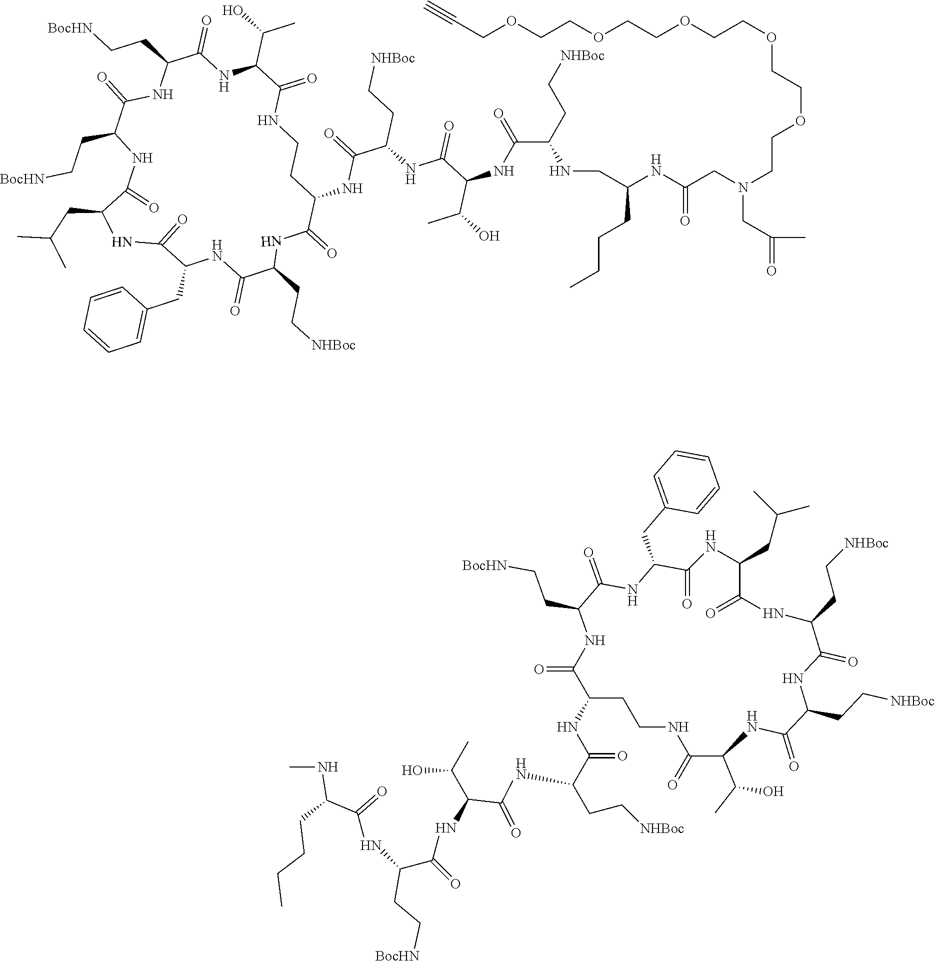

The compound from step f (50 mg, 0.067 mmol), and penta-Boc-polymyxin B decapeptide (240 mg, 0.15 mmol, 2.1 eq) in DMF (5 mL) were added EDC (60 mg, 0.3 mmol), HOBT (45 mg, 0.3 mmol), DIEA (0.14 mL, 1.0 mmol) at room temperature. The solution was stirred overnight then concentrated and re-dissolved into 2 mL methanol. This solution was added dropwise to 50 mL vigorously stirred water. This heterogeneous solution was stirred for 1 hour, then filtered with a Buchner funnel with Whatman filter paper. The crude product was used for next step without purification.

### Step h

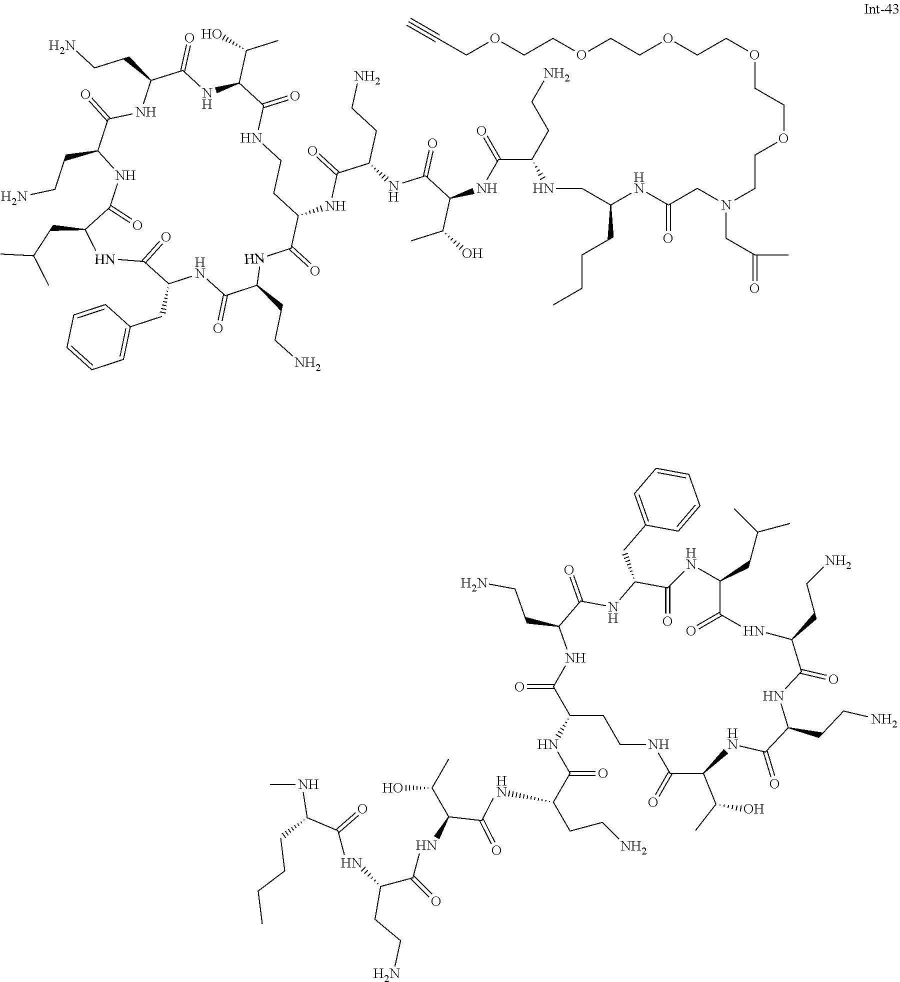 [SMILES: C#CCOCCOCCOCCOCCOCCN(CC(C)=O)CC(=O)N[C@@H](CCCC)CN[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O ; CCCC[C@H](NC)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O]

Intermediate from step g was treated with 2 mL DCM and 2 mL TFA at room temperature. The solution was stirred 10 min, then concentrated and purified by RPLC using an Isco CombiFlash liquid chromatograph eluted with 0% to 100% acetonitrile and water, using formic acid as the modifier. Yield of Int-43 was 0.080 g as TFA salt, 67% yield. Ion(s) found by LCMS: [M/4]+H+=715.7, [M/5]+H+=572.7, [M16]+H+=477.5, [M/7]+H+=409.4.

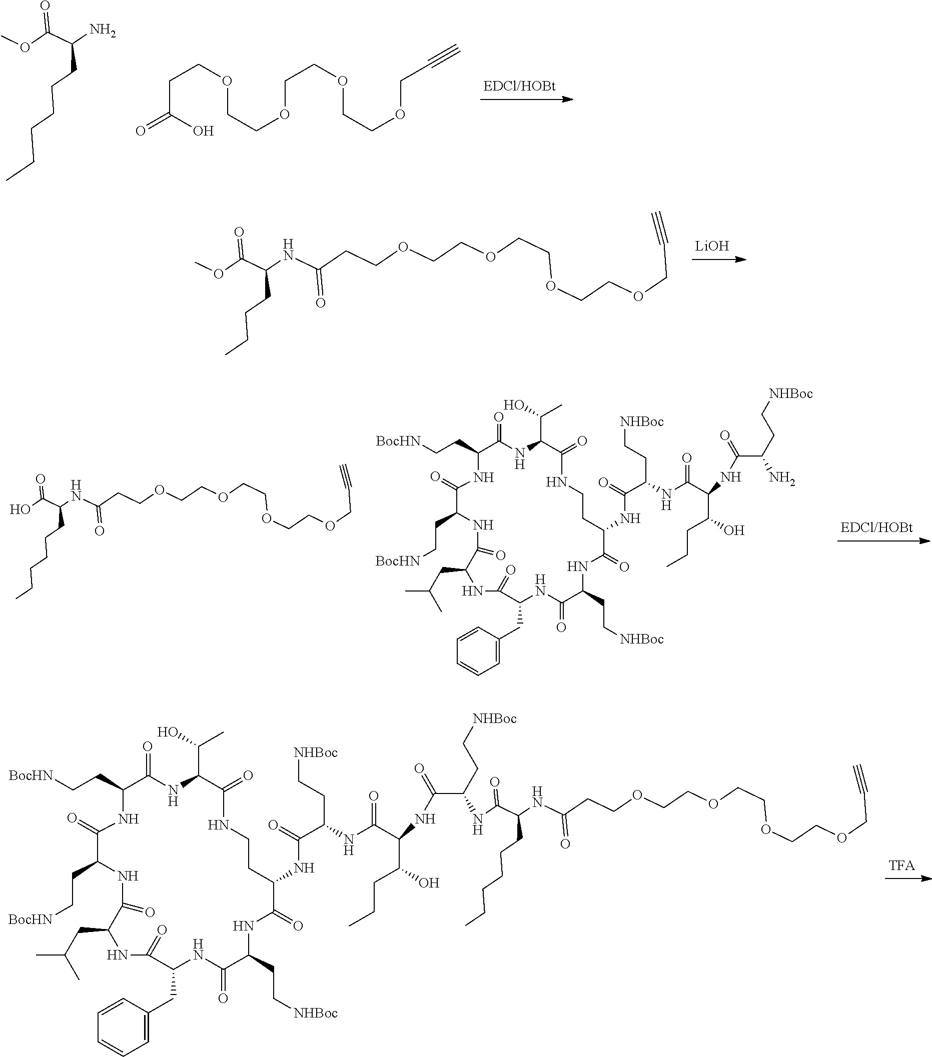

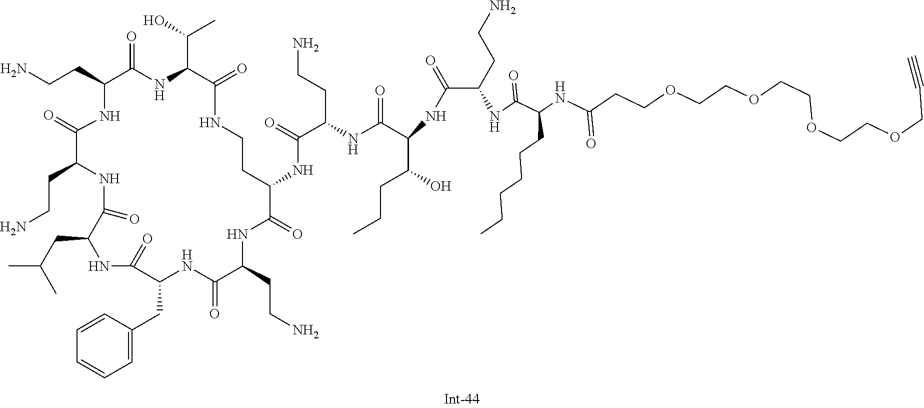 [SMILES: C#CCOCCOCCOCCOCCC(=O)N[C@@H](CCCCCC)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCC]

### Step a

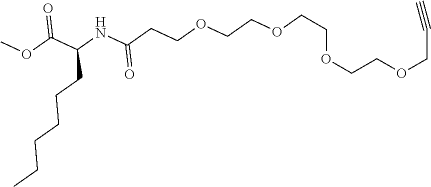 [SMILES: C#CCOCCOCCOCCOCCC(=O)N[C@@H](CCCCCC)C(=O)OC]

To a solution propargyl-PEG-4-acid (0.52 g 2 mmol), and methyl L-norleucinate (0.35 g, 2 mmol) in DMF (20 mL) was added EDC (0.60 g, 3 mmol), HOBT (0.45 g, 3 mmol), DIEA (0.58 mL, 4 mmol) at room temperature. The solution was stirred overnight. The resulting solution was concentrated and purified by RPLC using an Isco CombiFlash liquid chromatograph eluted with 5% to 100% acetonitrile and water, using 0.1% TFA as the modifier. Ion(s) found by LCMS: (M+H)+=416.2.

### Step b

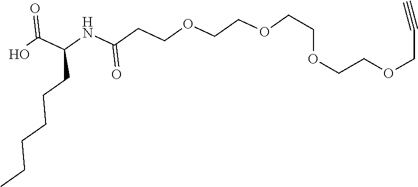 [SMILES: C#CCOCCOCCOCCOCCC(=O)N[C@@H](CCCCCC)C(=O)O]

Lithium hydroxide (60 mg, 2.5 mmol) in 2 mL water was added into a solution of the amino acid ester from the previous step (0.4 g, 1 mmol) in 2 mL methanol and 2 mL THF. The reaction was monitored by LCMS. After completion, the solution was adjusted to pH 1 with Amberlite® IRN-77, then the resulting solution was filtered and concentrated and used in next step without further purification. Ion(s) found by LCMS: (M+H)+=402.2.

### Step c

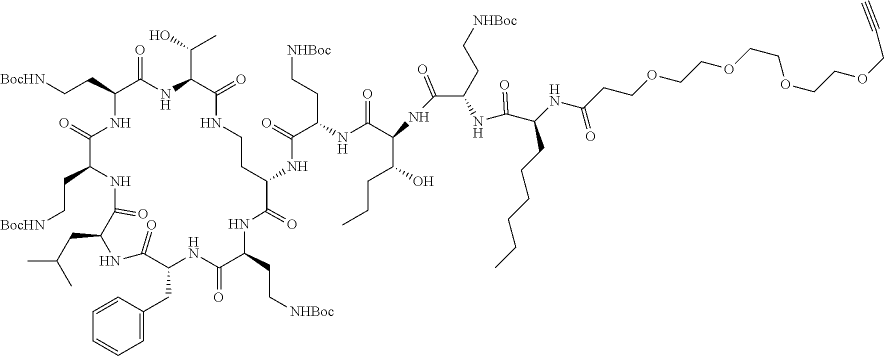 [SMILES: C#CCOCCOCCOCCOCCC(=O)N[C@@H](CCCCCC)C(=O)N[C@@H](CCNC(=O)OC(C)(C)C)C(=O)N[C@H](C(=O)N[C@@H](CCNC(=O)OC(C)(C)C)C(=O)N[C@H]1CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](Cc2ccccc2)NC(=O)[C@H](CCNC(=O)OC(C)(C)C)NC1=O)[C@H](O)CCC]

The compound from step d (40 mg, 0.1 mmol), and penta-Boc-polymyxin B decapeptide (160 mg, 0.1 mmol, 1 eq) in DMF (5 mL) were added EDC (40 mg, 0.2 mmol), HOBT (30 mg, 0.2 mmol), DIEA (0.14 mL, 1.0 mmol) at room temperature. The solution was stirred overnight. The resulting solution was concentrated and re-dissolved into 2 mL methanol. The resulting solution was added dropwise into 50 mL vigorously stirred water, this heterogeneous solution was stirred for 1 hour, then filtered with Buchner funnel with Filter paper. The crude product was used in next step without further purification.

### Step d

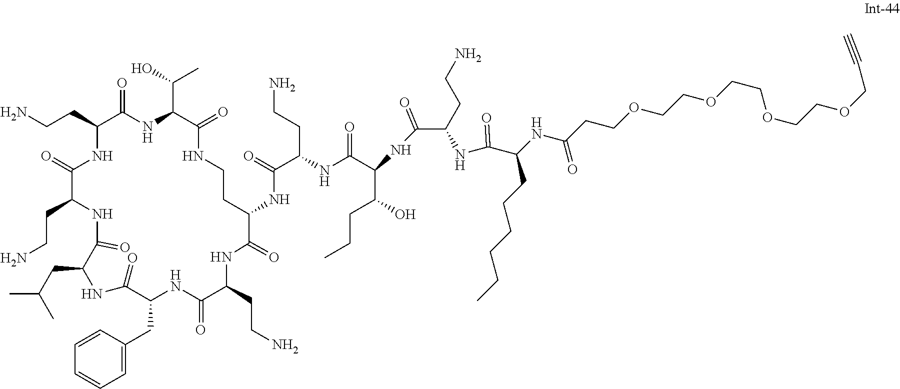 [SMILES: C#CCOCCOCCOCCOCCC(=O)N[C@@H](CCCCCC)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCC]

Intermediate form the previous step was treated with 2 mL C and 2 mL A at room temperature, stirred for 10 min, then concentrated and purified by RPLC using an Isco CombiFlash liquid chromatograph eluted with 0% to 100% acetonitrile and water, using formic acid as the modifier. Yield of Int-44 was 0.060 g, 42% yield. Ion(s) found by LCMS: [M/2]+H+=737.9, [M/3]+H+=492.3, [M/4]+H+=369.5.

### Example 80—Synthesis of Int-46

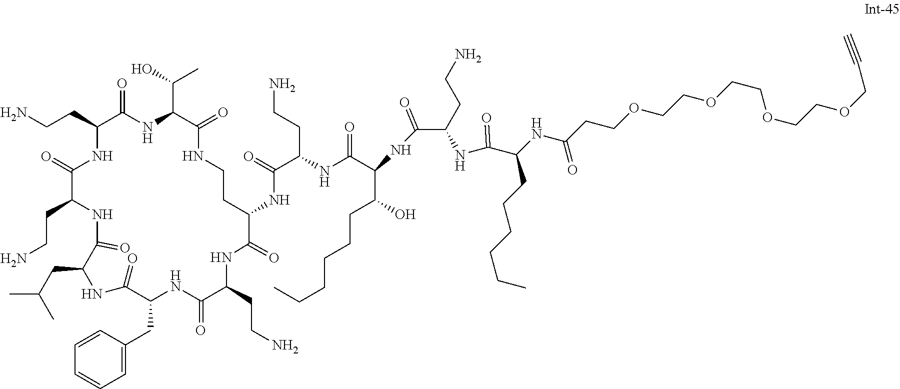 [SMILES: C#CCOCCOCCOCCOCCC(=O)N[C@@H](CCCCCC)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCCCCC]

The title compound Int-45 was prepared analogously to Example 79, where the where penta-Boc polymyxin B decapeptide was substituted with decapeptide described in Example 69, step c of the sequence. Ion(s) found by LCMS: [M12]+H+=758.9. [M13]+H+=506.3, [W/4]+H+=380.0.

### Example 81—Synthesis of Int-46

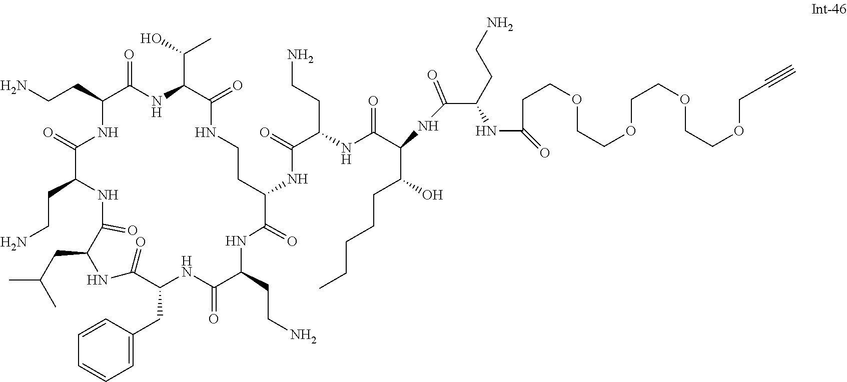 [SMILES: C#CCOCCOCCOCCOCCC(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCCCC]

The title compound Int-46 was prepared analogously to Examples 80, where the acid intermediate was substituted by propargyl-PEG-4-acid in step c of the sequence. Ion(s) found by LCMS: [M/2]+H+=681.4, [M/3]+H+=454.6.

### Example 82—Synthesis of Int-47

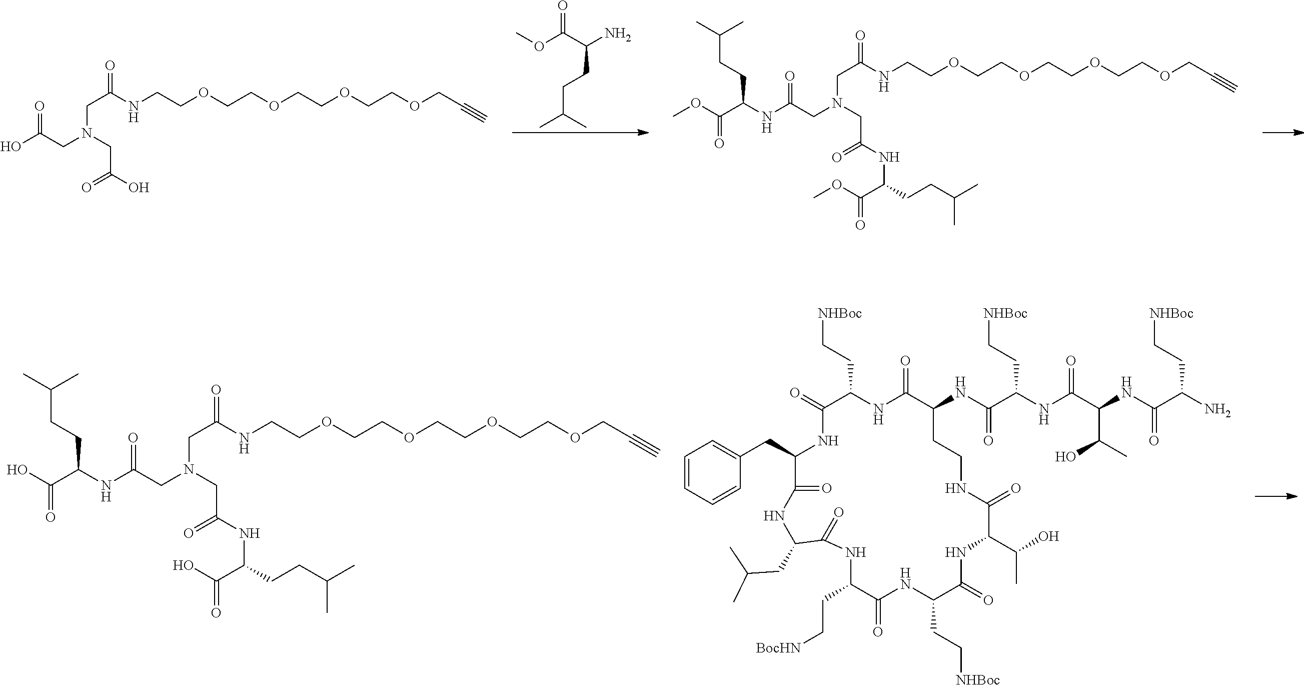

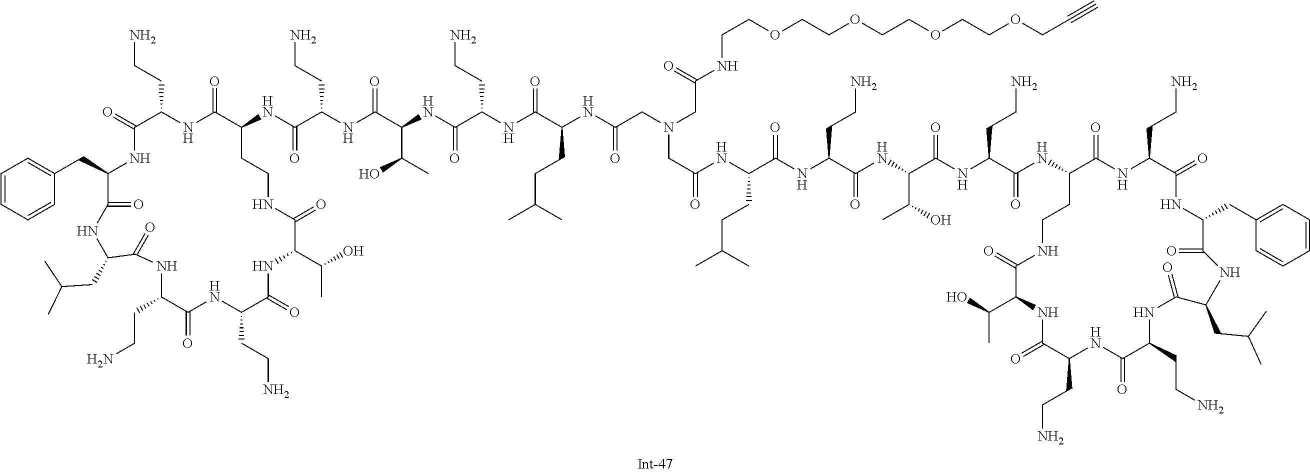 [SMILES: C#CCOCCOCCOCCOCCNC(=O)CN(CC(=O)N[C@@H](CCC(C)C)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O)CC(=O)N[C@@H](CCC(C)C)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O]

### Step a

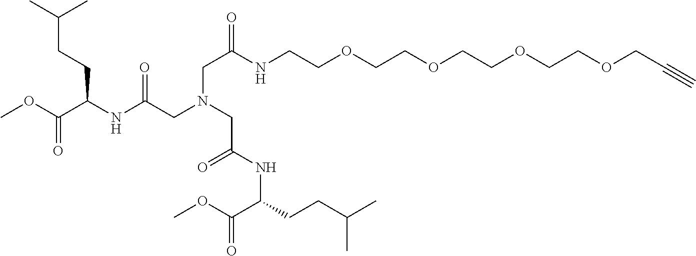 [SMILES: C#CCOCCOCCOCCOCCNC(=O)CN(CC(=O)N[C@H](CCC(C)C)C(=O)OC)CC(=O)N[C@H](CCC(C)C)C(=O)OC]

A flask was charged with and (S)-Methyl 2-amino-5-methylhexanoate (0.010 g, 0.627 mmol), diacid (0.115 g, 0.285 mmol, described in Example 90, step a), DIEA (0.40 mL, 2.28 mmol) in anhydrous DMF (1 mL). To this reaction mixture was added a solution of HATU (0.238 g, 0.627 mmol) in DMF (1 mL) at a rate of 2.5 mL/h. After the addition, the reaction mixture was stirred for an addition hour then was purified by RPLC using an Isco CombiFlash liquid chromatograph eluted with 5% to 95% acetonitrile and water, using 0.1% TFA as modifier. Yield of 0.126 g, 64%. Ion(s) found by LCMS: (M+H)+=687.4

### Step b

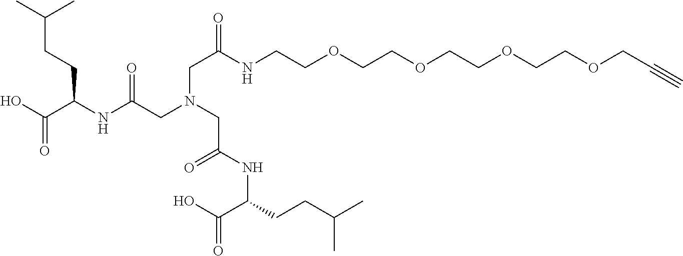 [SMILES: C#CCOCCOCCOCCOCCNC(=O)CN(CC(=O)N[C@H](CCC(C)C)C(=O)O)CC(=O)N[C@H](CCC(C)C)C(=O)O]

A solution of bis-ester product from the previous step (0.126 g, 0.183 mmol), dissolved in MeOH:THF:H₂O (1:1:2, 8 mL), was treated with lithium hydroxide solid (0.026 g, 1.10 mmol). The reaction mixture was continued stirring overnight. After the reaction was completed by monitoring with LCMS, the reaction was quenched with glacial acetic acid to pH 4, stripped of methanol and tetrahydrofuran then purified by RPLC using an Isco Combiflash liquid chromatograph eluted with 5% to 55% acetonitrile and water, using 0.1% TFA as modifier. Yield of 0.088 g, 72%. Ion(s) found by LCMS: (M+H)⁺=659.3

### Step c

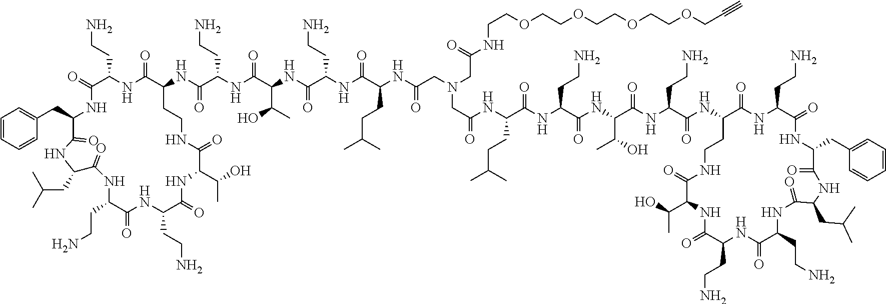 [SMILES: C#CCOCCOCCOCCOCCNC(=O)CN(CC(=O)N[C@@H](CCC(C)C)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O)CC(=O)N[C@@H](CCC(C)C)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O]

A flask was charged with di-acid product from the previous step (0.048 g, 0.073 mmol), HOAt (0.030 g, 0.220 mmol), EDC (0.042 g, 0.220 mmol) and Int-7a (0.252 g, 0.161 mmol, Example 25, step a) in DMF (2 mL). The reaction mixture was stirred at room temperature for 10 minutes followed by addition of DIEA (0.08 mL, 0.439 mmol) then continued to stir overnight. The deca-Boc-protected intermediate was purified by RPLC using an Isco Combiflash liquid chromatograph eluted with 5% to 25% then 100% methanol and water, using 0.1% TFA as modifier. Pure fractions were combined and concentrated to yield deca-boc-protected intermediate.

A solution of deca-Boc-protected intermediate from the previous step was stirred in TFA/CH₂Cl₂ (1:1, 6 mL) at ambient temperature for 1 hour then stripped of TFA and CH₂Cl₂ using the rotary evaporator. The product was purified by RPLC using an Isco Combiflash liquid chromatograph eluted with 5% to 30% acetonitrile and water, using 0.1% TFA as modifier. Yield of 0.087 g as TFA salt, 31%, 2 steps. Ion(s) found by LCMS: [(M+5H)/5]+=551.20, [(M+6H)/6]+=458.9

### Example 83—Synthesis of Int-48

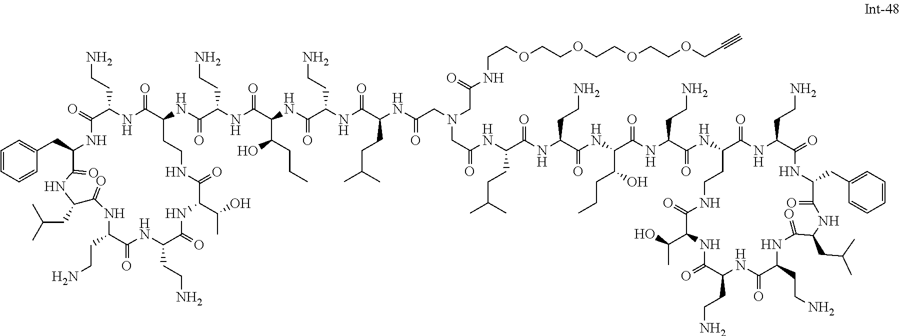 [SMILES: C#CCOCCOCCOCCOCCNC(=O)CN(CC(=O)N[C@@H](CCC(C)C)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCC)CC(=O)N[C@@H](CCC(C)C)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCC]

The title compound Int-48 was prepared from the di-acid product from the Example 82, step a (0.04 g, 0.061 mmol) and the extended threonine decapeptide (0.213 g, 0.134 mmol, Example 67, step h) as described in Example 67, step m. Yield 0.0767 g as TFA salt, 32%, 2 steps. Ion(s) found by LCMS: [(M+5H)/5]+=561.8, [(M+6H)/6]+=468.6.

### Example 64—Synthesis of Int-49

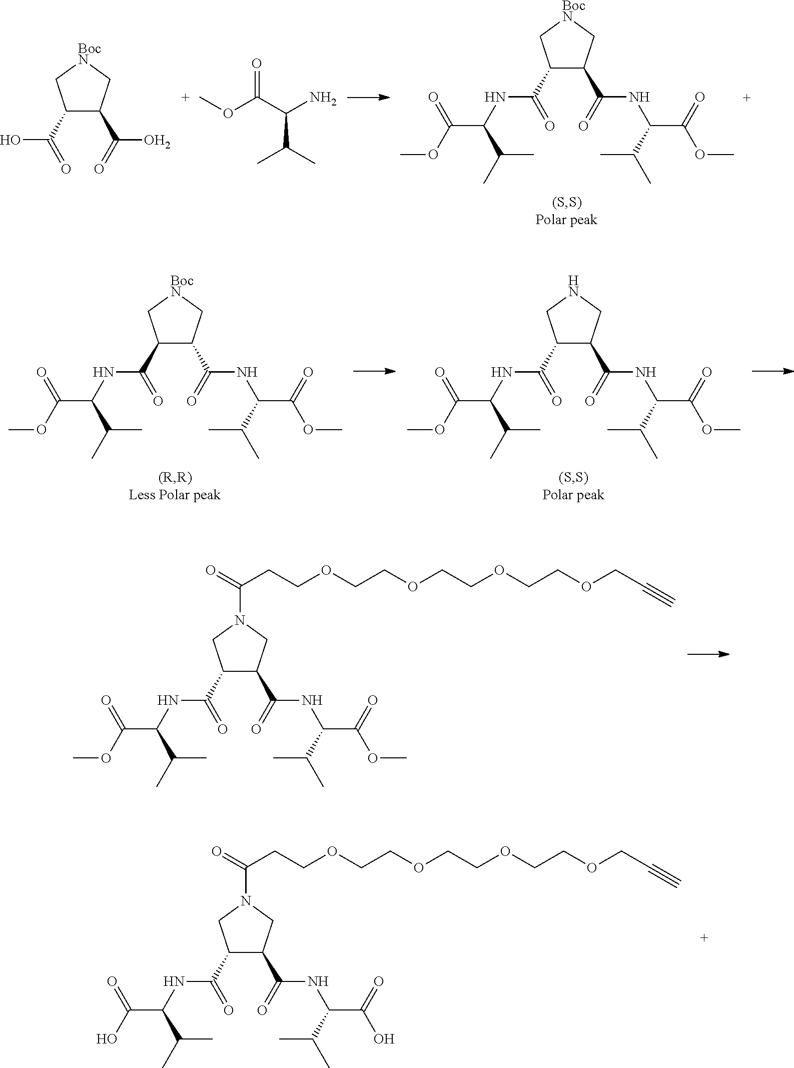

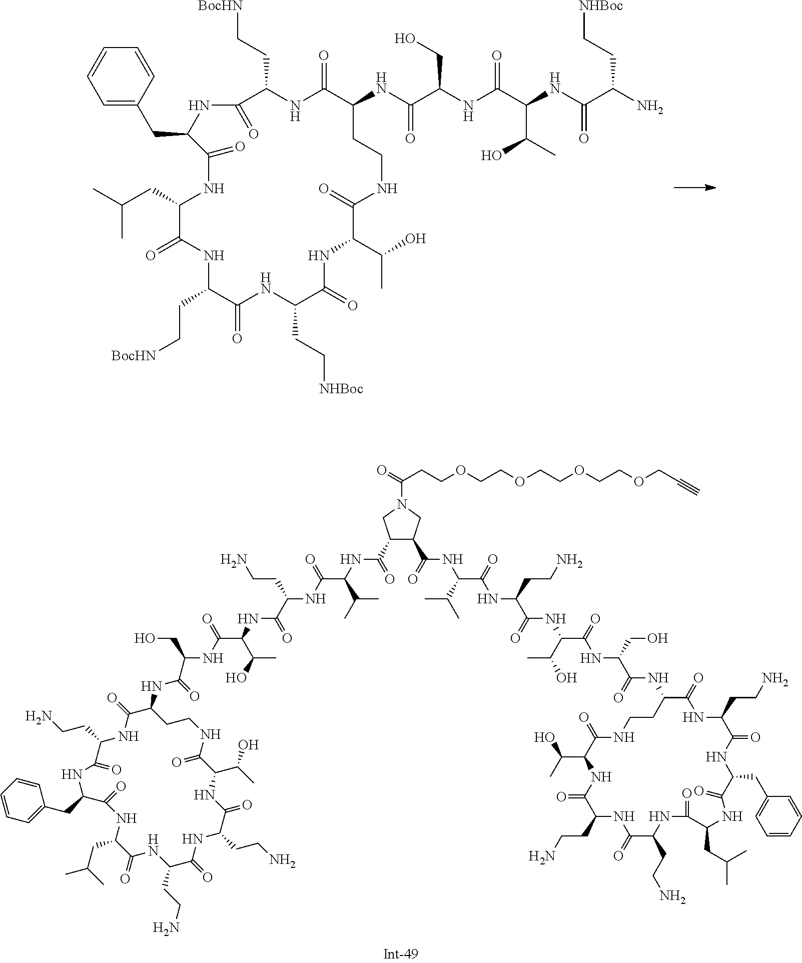

### Step a

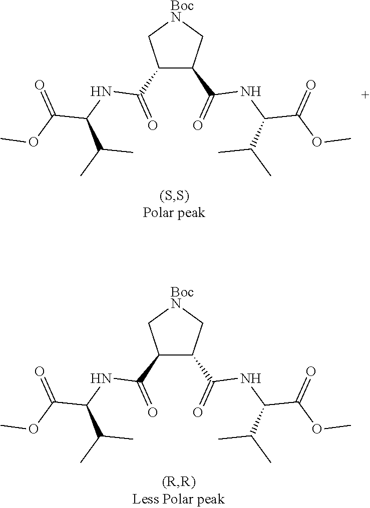

A flask was charged with L-valine methyl ester HCl (0.970 g, 5.786 mmol), racemic-trans-(tert-butoxycarbonyl)pyrrolidine-3,4-dicarboxylic acid (0.500 g, 1.929 mmol), DIEA (2.0 mL, 11.57 mmol) in anhydrous DMF (8 mL). To this reaction mixture was added a solution of HATU (2.2 g, 5.86 mmol) in DMF (8 mL) at a rate of 4 mL/h. After stirring overnight solvent was stripped to half of the volume then purified by RPLC using an Isco Combiflash liquid chromatograph eluted with 5% to 45% acetonitrile and water, using 0.1% TFA as modifier. Yield of 0.0845 g polar diastereomer 9% (used in the next step). Yield of less polar isomer 0.18 g, 14% yield (discarded). Ion(s) found by LCMS: (M−Boc)+=386.2

### Step b

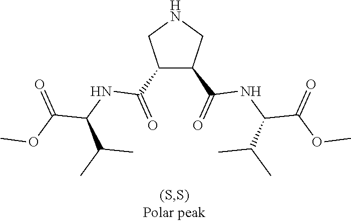

A solution of the Boc-Pyrrolidine product from the previous step (0.085 g, 0.174 mmol) in dioxane (3 mL) was charged with HCl (4M in dioxane, 2 mL). The reaction mixture was stirred at ambient temperature for 1 hour then concentrated under reduced pressure. The residue was rinsed with ethyl acetate (20 mL, 2×) then concentrated and dried under reduced pressure. Yield 0.160 g as HCl salt, 189%. Ion(s) found by LCMS: [M+H]+=386.0.

### Step c

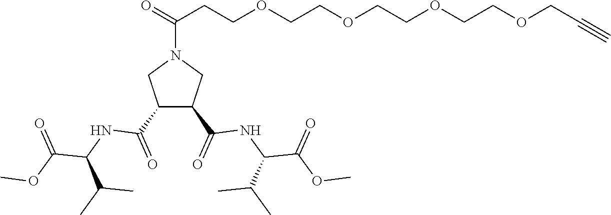 [SMILES: C#CCOCCOCCOCCOCCC(=O)N1C[C@@H](C(=O)N[C@H](C(=O)OC)C(C)C)[C@H](C(=O)N[C@H](C(=O)OC)C(C)C)C1]

A flask was charged with propargyl-PEG4-acid (0.054 g, 0.19 mmol), the product from the previous step (0.073 g, 0.173 mmol) and DIEA (0.09 mL, 0.519 mmol) was stirred at ambient temperature in DMF (1 mL). To this solution was added a solution of HATU (0.072 g, 0.19 mmol) in DMF (0.6 mL) via a syringe pump at a rate of 2.5 mL/hr. After stirring for 1 hr product was purified by RPLC using an Isco Combiflash liquid chromatograph eluted with 5% to 50% acetonitrile and water, using 0.1% TFA as modifier. Yield 0.114 g, 105%. Ion(s) found by LCMS: (M+H)+=628.4

### Step d

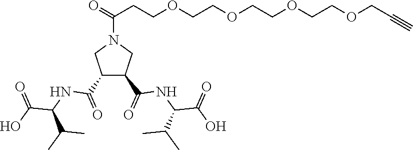 [SMILES: C#CCOCCOCCOCCOCCC(=O)N1C[C@@H](C(=O)N[C@H](C(=O)O)C(C)C)[C@H](C(=O)N[C@H](C(=O)O)C(C)C)C1]

A solution of bis-ester from the previous step (0.10 g, 0.159 mmol), dissolved in MeOH:THF:H₂O (1:1:2, 8 mL), was treated with lithium hydroxide solid (0.011 g, 0.478 mmol). The reaction mixture was continued stirring overnight. After the reaction was completed by monitoring with LCMS, the reaction was quenched with glacial acetic acid to pH 4, stripped of methanol and tetrahydrofuran then purified by RPLC using an Isco Combiflash liquid chromatograph eluted with 5% to 45% acetonitrile and water, using 0.1% TFA as modifier. Yield of 0.073 g, 77%. Ion(s) found by LCMS: (M+H)+=598.2

### Step e

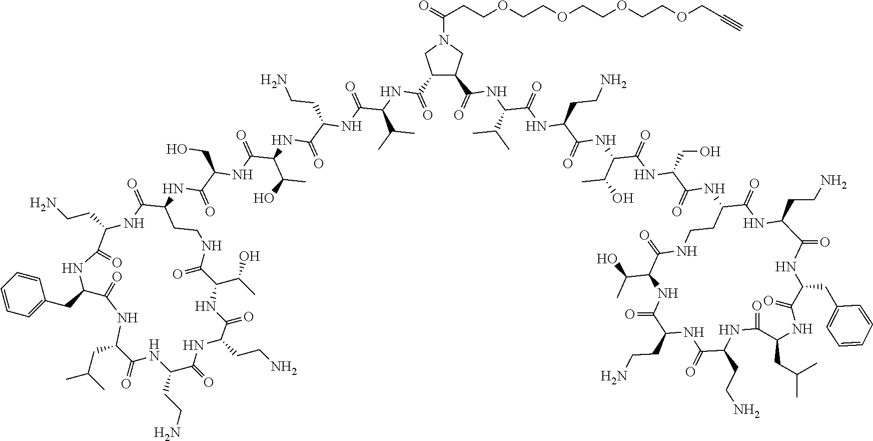 [SMILES: C#CCOCCOCCOCCOCCC(=O)N1C[C@@H](C(=O)N[C@H](C(=O)N[C@@H](CCN)C(=O)N[C@H](C(=O)N[C@H](CO)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)C(C)C)[C@H](C(=O)N[C@H](C(=O)N[C@@H](CCN)C(=O)N[C@H](C(=O)N[C@H](CO)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)C(C)C)C1]

A 4 mL scintillation vial was charged with di-acid product from the previous step (0.073 g, 0.122 mmol), HOAt (0.051 g, 0.367 mmol), EDC (0.070 g, 0.367 mmol) and PMB-decapeptide-D-Ser (0.390 g, 0.269 mmol, Example 6, Int-5d) in DMF (2 mL). The reaction mixture was stirred at room temperature for 10 minutes followed by addition of DIEA (0.13 mL, 0.733 mmol), then continued to stir overnight. Crude product was purified by RPLC using an Isco Combiflash liquid chromatograph eluted with 5% to 25% then 100% methanol and water, using 0.1% TFA as modifier. Pure fractions were combined and concentrated to yield deca-boc-protected intermediate.

A solution of deca-Boc-protected intermediate from the previous step was stirred in TFA/CH₂Cl₂ (1:1, 6 mL) at ambient temperature for 1 hour then stripped of TFA and CH₂Cl₂ using a rotary evaporator. The product was purified by RPLC using an Isco Combiflash liquid chromatograph eluted with 5% to 30% acetonitrile and water, using 0.1% TFA as modifier. Yield of Int-49 was 0.131 g as a TFA salt, 30%, 2 steps. Ion(s) found by LCMS: [(M+4)/4]+=667.0, [(M+5H)/5]+=534.0. [(M+6H)/6]+=445.0

### Example 85—Synthesis of Int-50

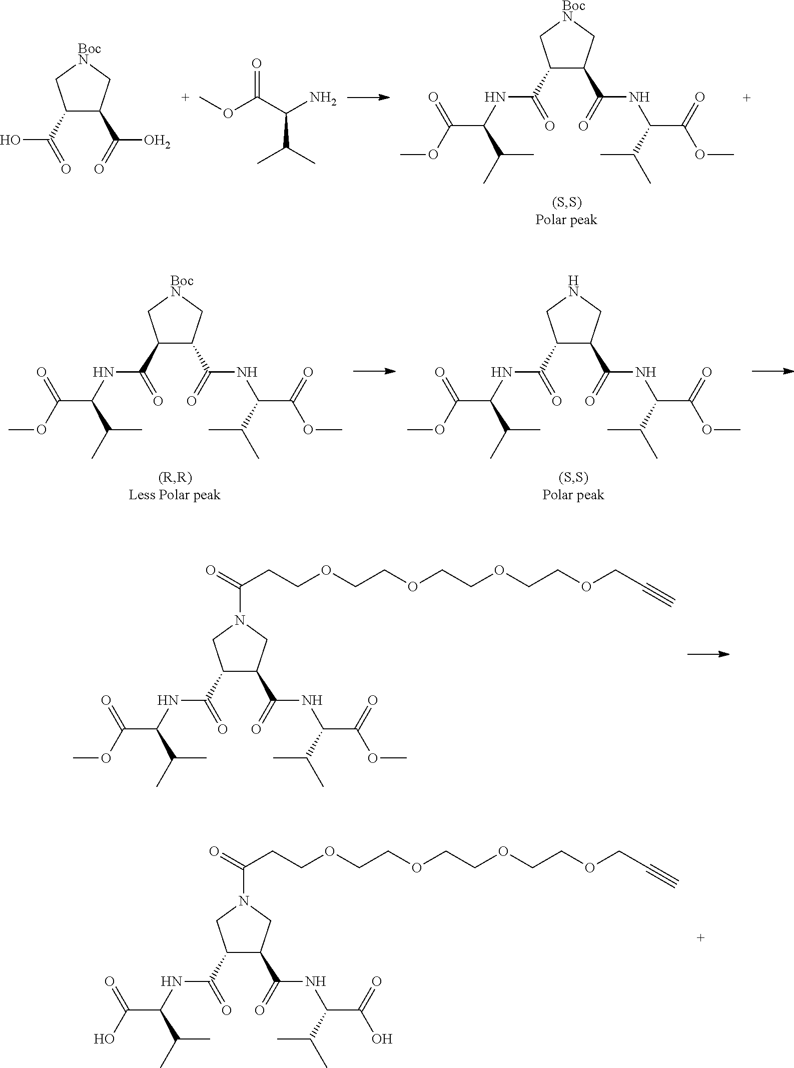

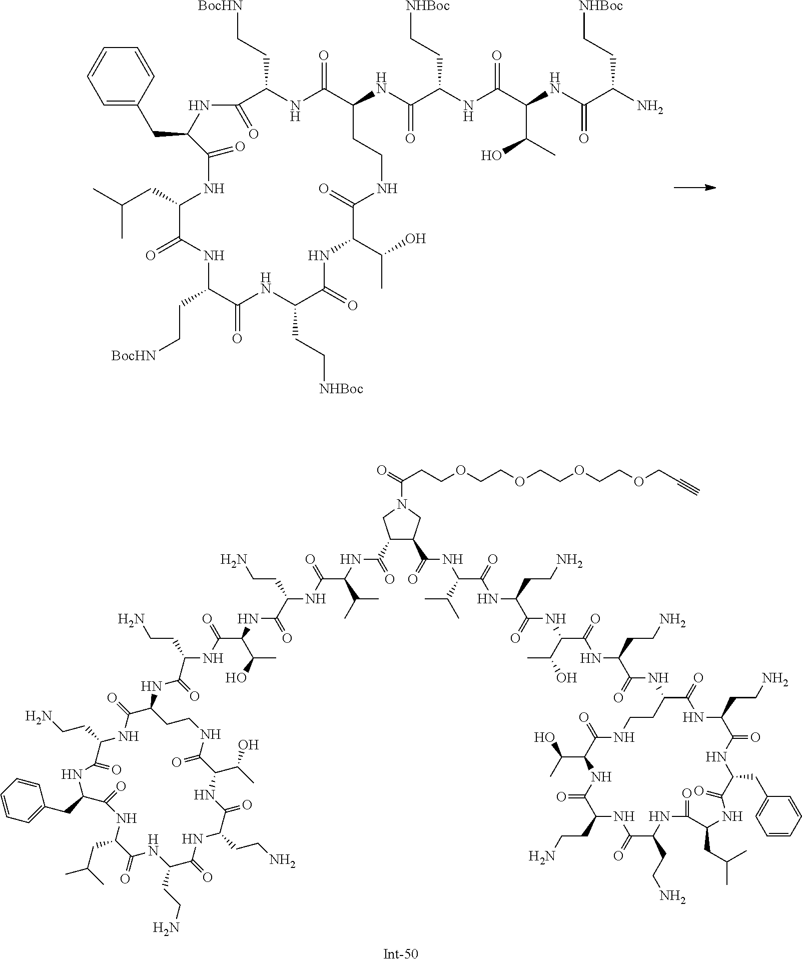

### Step a

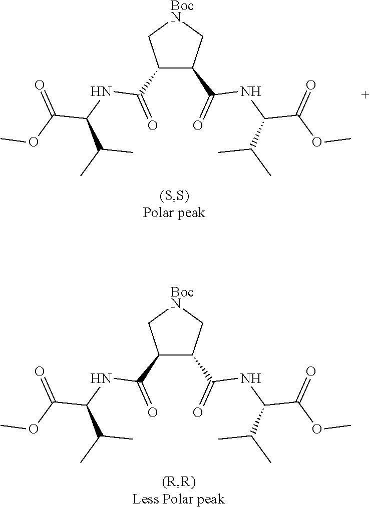

A flask was charged with L-Valine methyl ester HCl (0.776 g, 4.63 mmol), racemic-trans1-(tert-butoxycarbonyl)pyrrolidine-3,4-dicarboxylic acid (0.40 g, 1.543 mmol), HOAt (0.861 g, 6.326 mmol), and EDC (1.21 g, 6.326 mmol) in anhydrous DMF (6 mL). The reaction mixture was stirred at ambient temperature for 10 minutes following by addition of DIEA (2.42 mL, 13.89 mmol) then continued to stir overnight. The reaction mixture was reduced by half using a rotatory evaporator, then purified by RPLC using an Isco CombiFlash liquid chromatograph eluted with 5% to 50% acetonitrile and water, using 0.1% TFA as modifier. Yield 0.144 g, 19% of the polar diastereomer (less polar isomer discarded) as a clear oil. Ion(s) found by LCMS: (M−Boc)+=386.2 and (M−H)+=484.2

Steps b, c & d.

The diacid was prepared as described in Example 84, steps b, c, d. Yield 0.17 g, 135% yield; ion(s) found by LCMS: [M+H]+=385.2 (Step b). Yield 0.201 g, 79% yield; ion(s) found by LCMS: [M+H]+=628.4 (Step c). Yield 0.138 g, 72% yield; ion(s) found by LCMS: [M+H]+=601.3 (Step d).

### Step e

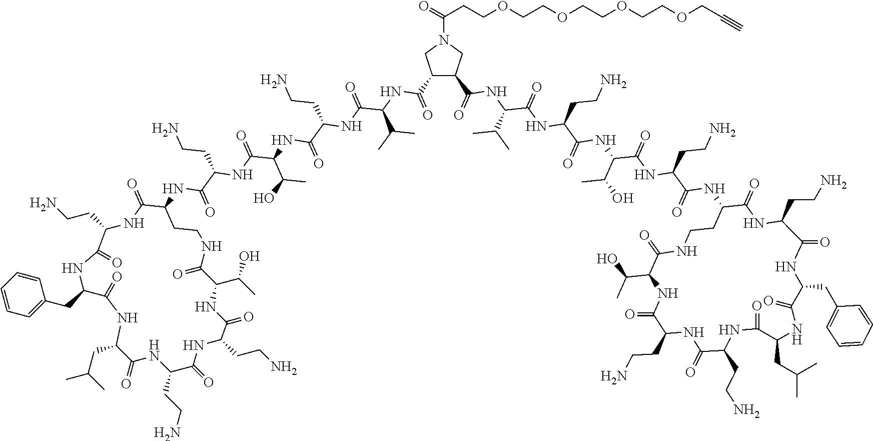 [SMILES: C#CCOCCOCCOCCOCCC(=O)N1C[C@@H](C(=O)N[C@H](C(=O)N[C@@H](CCN)C(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)C(C)C)[C@H](C(=O)N[C@H](C(=O)N[C@@H](CCN)C(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)C(C)C)C1]

The title compound Int-50 was prepared from the di-acid product of step d (0.069 g, 0.115 mmol) and Int-7a (0.395 g, 0.253 mmol, Example 10, step a) following the procedure described in Example 84, step e. Yield 0.042 g as TFA salt, 10%, 2 steps. Ion(s) found by LCMS: [(M+5H)/5]+=539.6, [(M+6H)/6]+=449.4.

### Example 86—Synthesis of Int-51

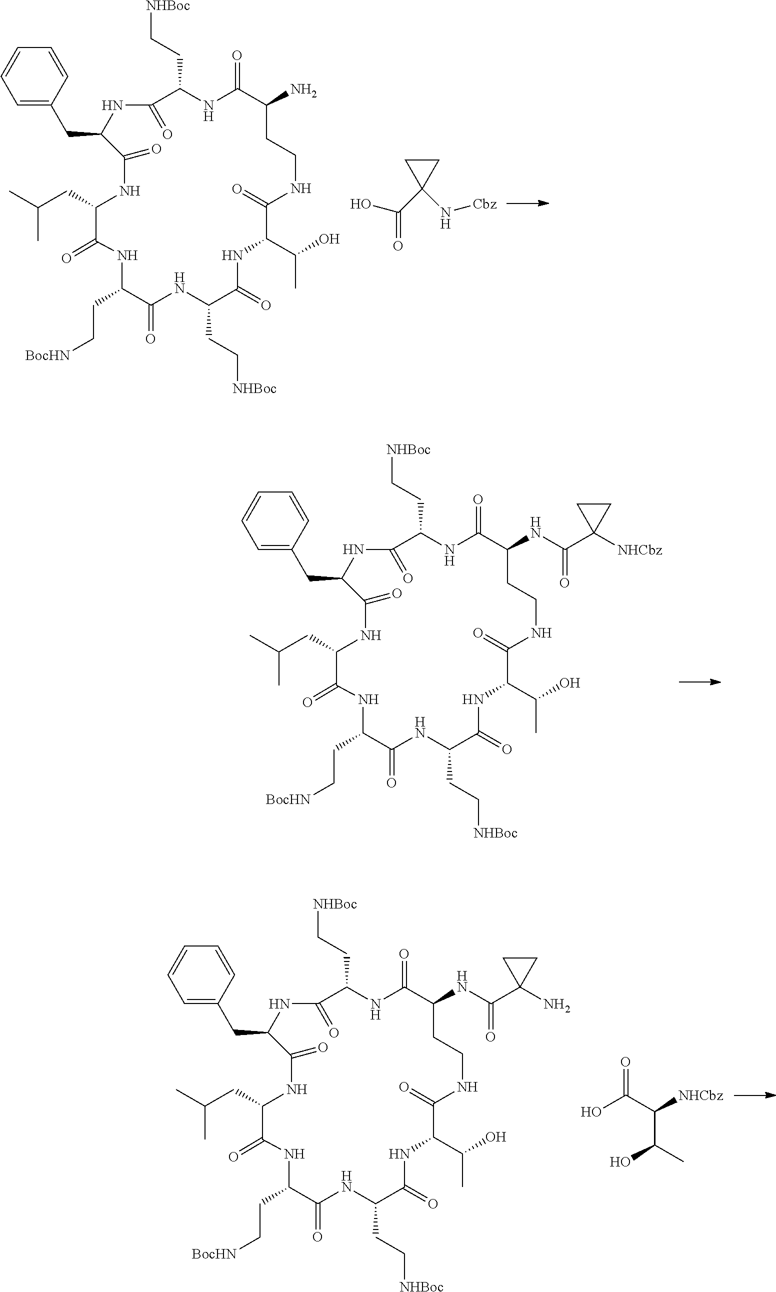

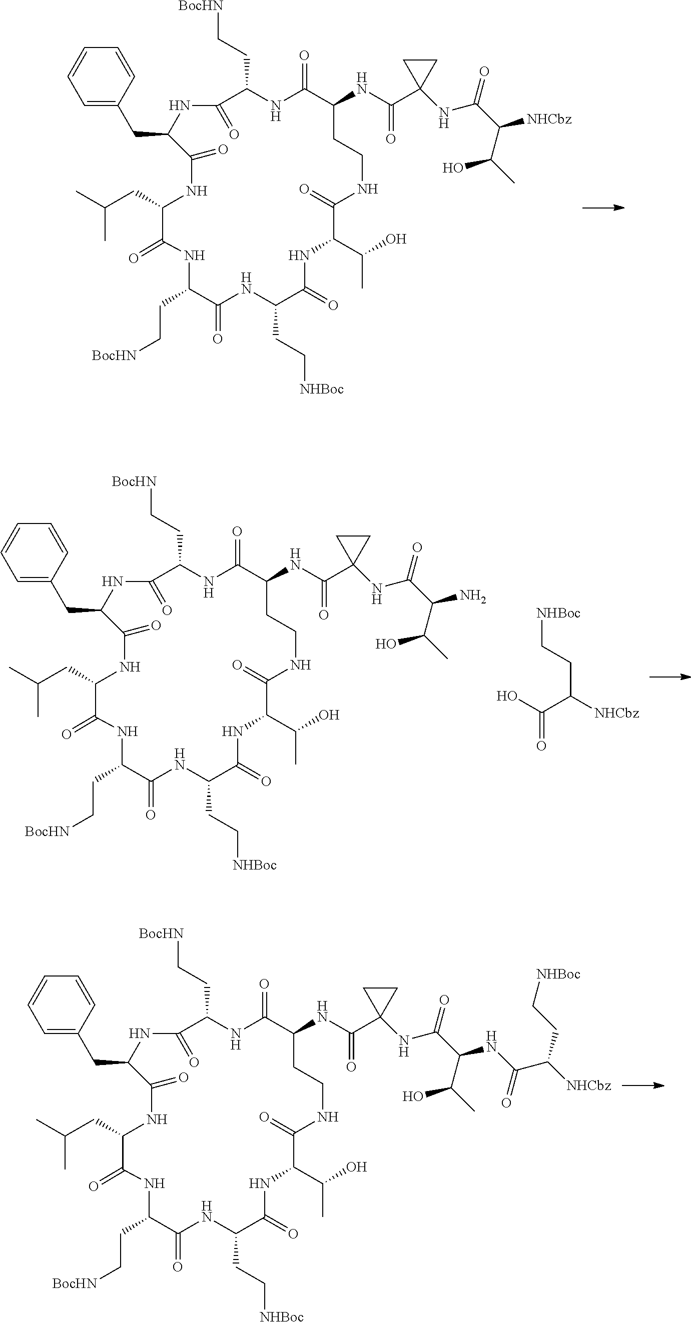

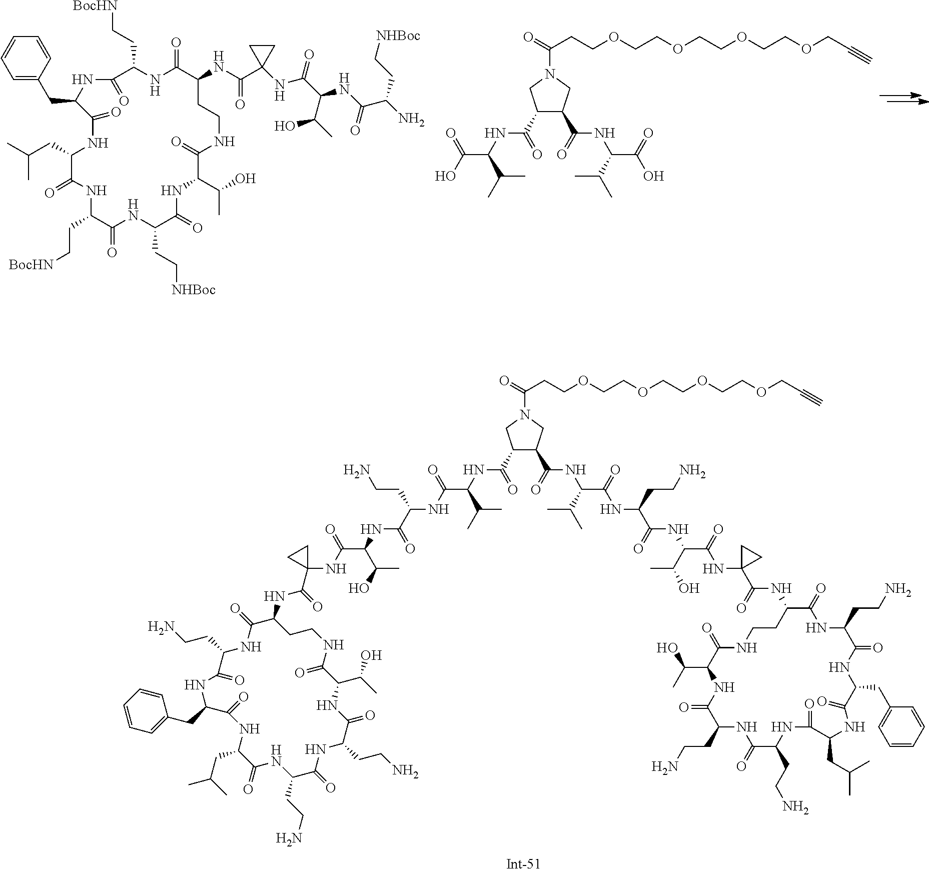

### Step a

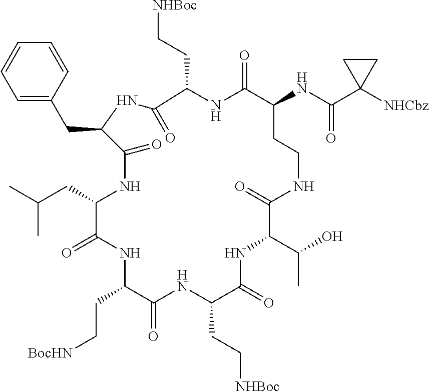 [SMILES: CC(C)C[C@@H]1NC(=O)[C@@H](Cc2ccccc2)NC(=O)[C@H](CCNC(=O)OC(C)(C)C)NC(=O)[C@@H](NC(=O)C2(NC(=O)OCc3ccccc3)CC2)CCNC(=O)[C@H]([C@@H](C)O)NC(=O)[C@H](CCNC(=O)OC(C)(C)C)NC(=O)[C@H](CCNC(=O)OC(C)(C)C)NC1=O]

A flask was charged with tri-Boc-PMBH-(NH₂) (2.00 g, 1.883 mmol, Example 2, Int-1), 1-Cbz-aminocyclopropane carboxylic acid (0.542 g, 2.259 mmol) and DIEA (0.98 mL, 5.65 mmol) in DMF (19 mL). To this solution was added a solution of HATU (0.877 g, 2.259 mmol) in DMF (3.5 mL) via a syringe pump at rate of 2.5 mL/hr. The reaction was stirred overnight at ambient temperature. The reaction mixture concentrated to half of its volume using the rotary evaporator then purified by RPLC using an Isco CombiFlash eluting with 5% to 25% to 90% methanol and water, using 0.1% TFA as modifier. Yield 2.159 g, 90%. Ion(s) found by LCMS [(M−2Boc+2H)/2]+=540.5.

### Step b

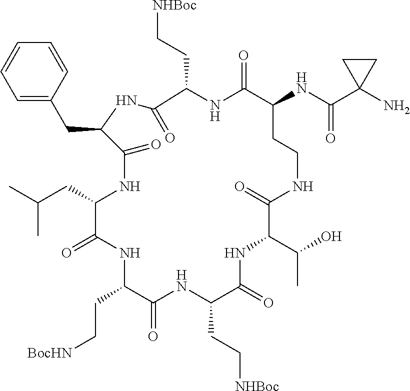 [SMILES: CC(C)C[C@@H]1NC(=O)[C@@H](Cc2ccccc2)NC(=O)[C@H](CCNC(=O)OC(C)(C)C)NC(=O)[C@@H](NC(=O)C2(N)CC2)CCNC(=O)[C@H]([C@@H](C)O)NC(=O)[C@H](CCNC(=O)OC(C)(C)C)NC(=O)[C@H](CCNC(=O)OC(C)(C)C)NC1=O]

A solution of the product from the previous step (2.08 g, 1.62 mmol) in methanol (15 mL) was charged with Pd/SiO₂ (0.405 g, 0.081 mmol) and hydrogen gas balloon. The reaction mixture was stirred at ambient temperature overnight. It was filtered through a pad of Celite® and washed with methanol, then concentrated to the title compound as white solid. Yield 1.94 g, 104%. Ion(s) was found by LCMS: [(M−2Boc+2H)/2]+=473.4, [(M−3Boc+2H)/2]+=423.3

### Step c

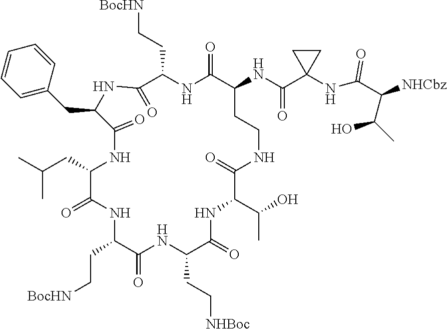 [SMILES: CC(C)C[C@@H]1NC(=O)[C@@H](Cc2ccccc2)NC(=O)[C@H](CCNC(=O)OC(C)(C)C)NC(=O)[C@@H](NC(=O)C2(NC(=O)[C@@H](NC(=O)OCc3ccccc3)[C@@H](C)O)CC2)CCNC(=O)[C@H]([C@@H](C)O)NC(=O)[C@H](CCNC(=O)OC(C)(C)C)NC(=O)[C@H](CCNC(=O)OC(C)(C)C)NC1=O]

A flask was charged with product from the previous step (0.979 g, 0.855 mmol), N-Cbz-L-threonine (0.227 g, 0.898 mmol), HOAt (0.175 g, 1.282 mmol) and EDC (0.246 g, 1.28 mmol) in DMF (2.6 mL) then stirred for 10 minutes following by addition of DIEA (0.45 mL, 2.56 mmol). The reaction mixture was stirred at ambient temperature overnight. It was purified by RPLC using an Isco CombiFlash eluting with 5% to 25% then 100% acetonitrile and water, using 0.1% TFA as modifier. Yield 0.84 g, 71%. Ion(s) found by LCMS: [(M−2Boc+2H)/2]+=590.8

### Step d

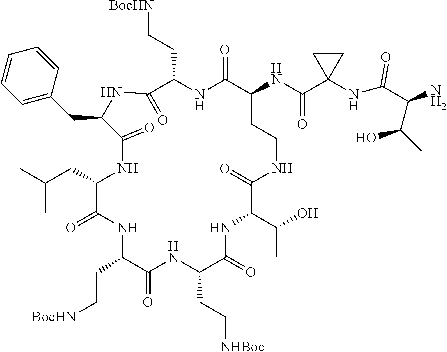 [SMILES: CC(C)C[C@@H]1NC(=O)[C@@H](Cc2ccccc2)NC(=O)[C@H](CCNC(=O)OC(C)(C)C)NC(=O)[C@@H](NC(=O)C2(NC(=O)[C@@H](N)[C@@H](C)O)CC2)CCNC(=O)[C@H]([C@@H](C)O)NC(=O)[C@H](CCNC(=O)OC(C)(C)C)NC(=O)[C@H](CCNC(=O)OC(C)(C)C)NC1=O]

The title compound was prepared from the product of the previous step using the procedure described in Step b of this example. Yield 0.725 g, 96%. Ion(s) found by LCMS: [M−2Boc+2H)/2]+=523.6

### Step e

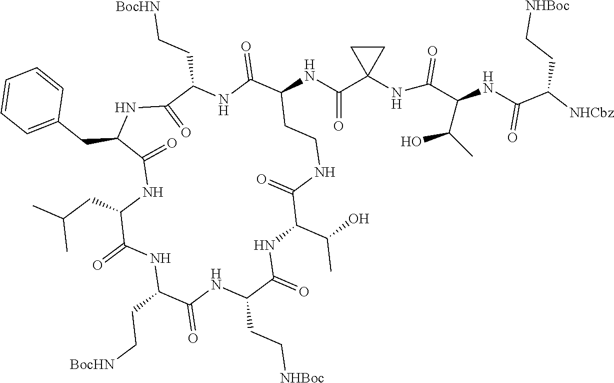 [SMILES: CC(C)C[C@@H]1NC(=O)[C@@H](Cc2ccccc2)NC(=O)[C@H](CCNC(=O)OC(C)(C)C)NC(=O)[C@@H](NC(=O)C2(NC(=O)[C@@H](NC(=O)[C@H](CCNC(=O)OC(C)(C)C)NC(=O)OCc3ccccc3)[C@@H](C)O)CC2)CCNC(=O)[C@H]([C@@H](C)O)NC(=O)[C@H](CCNC(=O)OC(C)(C)C)NC(=O)[C@H](CCNC(=O)OC(C)(C)C)NC1=O]

The title compound was prepared from the product of the previous (0.725 g, 0.581 mmol) and Z-dab(□Boc)-OH DCHA salt (0.341 g, 0.639 mmol) as described in procedure from Step c. Yield 0.626 g, 68%. Ion(s) found by LCMS: [(M−2Boc+2H)/2]+=690.4

### Step f

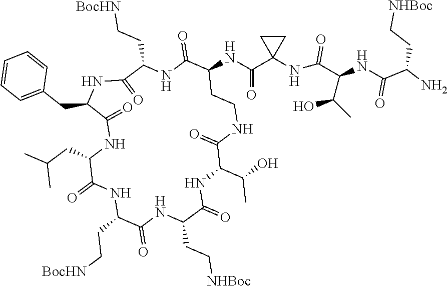 [SMILES: CC(C)C[C@@H]1NC(=O)[C@@H](Cc2ccccc2)NC(=O)[C@H](CCNC(=O)OC(C)(C)C)NC(=O)[C@@H](NC(=O)C2(NC(=O)[C@@H](NC(=O)[C@@H](N)CCNC(=O)OC(C)(C)C)[C@@H](C)O)CC2)CCNC(=O)[C@H]([C@@H](C)O)NC(=O)[C@H](CCNC(=O)OC(C)(C)C)NC(=O)[C@H](CCNC(=O)OC(C)(C)C)NC1=O]

The title compound was prepared from the product of the previous step (0.626 g, 0.396 mmol) and Pd/SiO₂ (0.099 g, 0.02 mmol) as described in Step b of this example. Yield 0.575 g, 100%. Ion(s) found by LCMS: (M+Na)+=646.0

### Step g

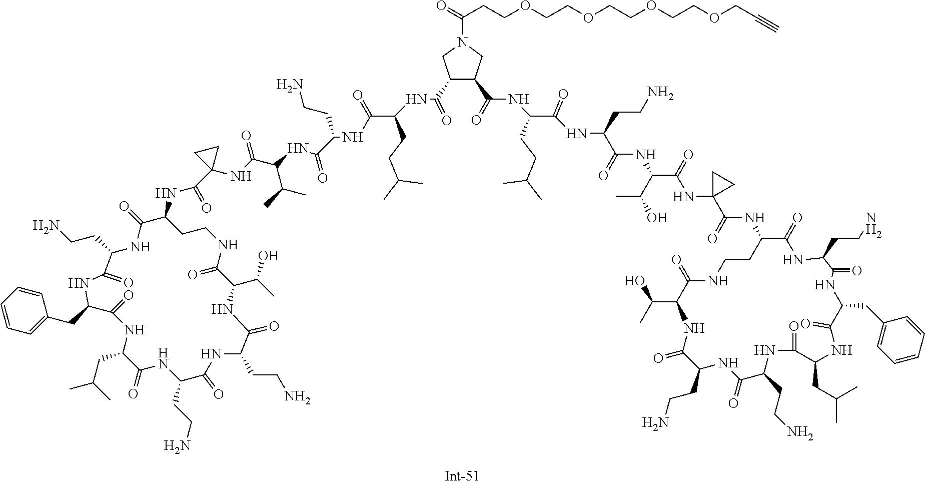

The title compound Int-51 was prepared from intermediate from Example 84, step d (0.069 g, 0.115 mmol) and intermediate from the previous step (0.366 g, 0.253 mmol), following the procedure from Example 84, step e. Yield 0.0478 g as TFA salt, 12%, 2 steps. Ion(s) found LCMS: [(M+4H)/4]+=664.8, [(M+5H)/5]+=532.0, [(M+6H)/6]+=443.6.

### Example 87—Synthesis of Int-52

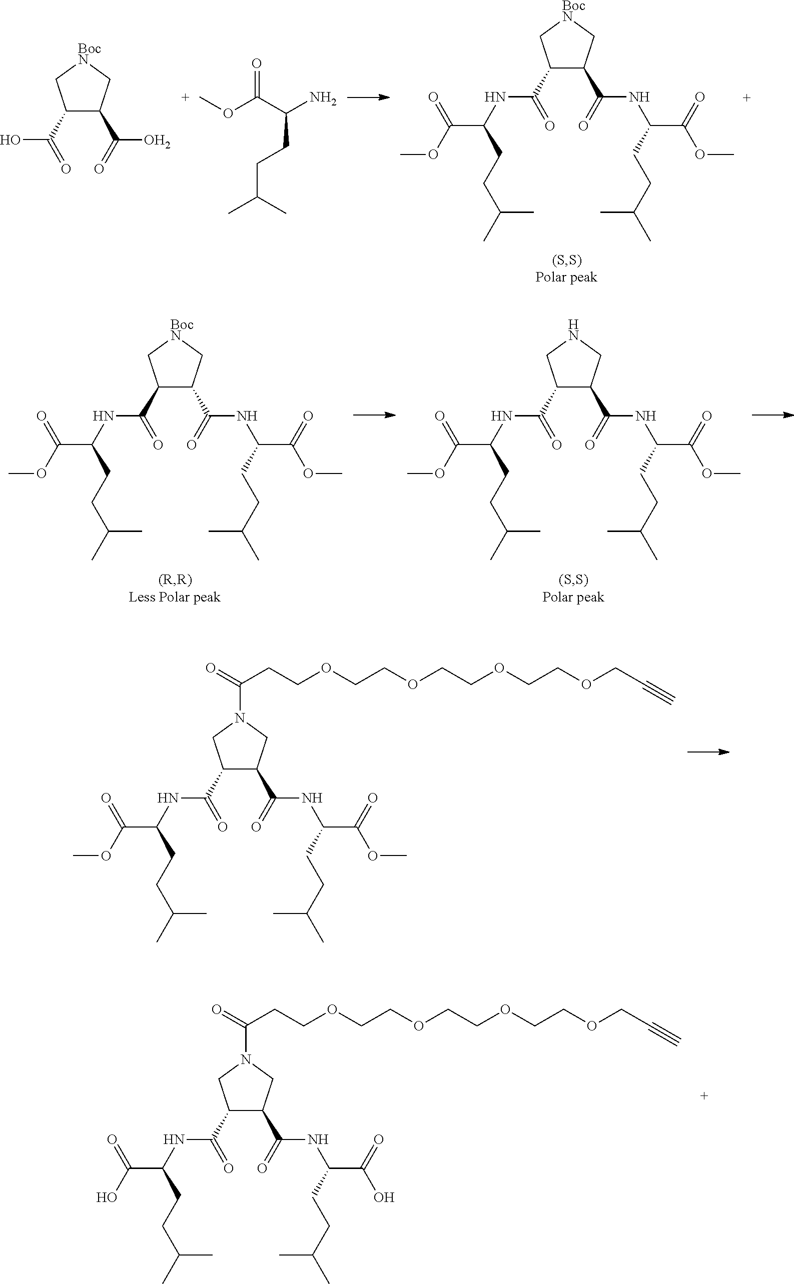

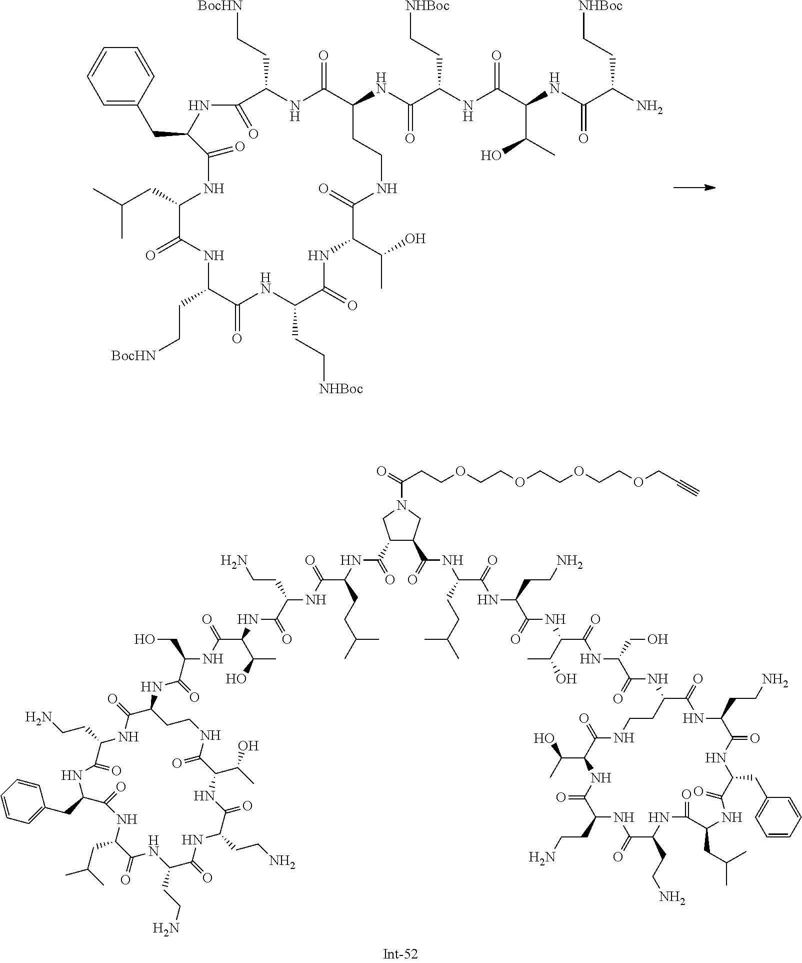

### Step a

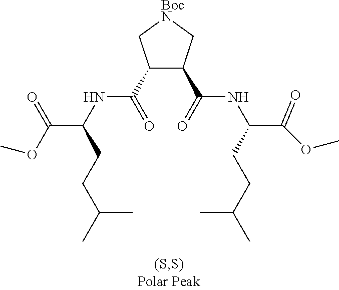

The diester was prepared from methyl (2S)-2-amino-5-methylhexanoate HCl salt (0.557 g, 2.85 mmol) and racemic-trans-1-(tert-butoxycarbonyl)pyrrolidine-3,4-dicarboxylic acid (0.352 g, 1.36 mmol) as described in Example 84, step a. Yield 0.303 g of the polar diastereomer 41%. Ion(s) found by LCMS: [M-Boc+H]+=442.2.

### Step b

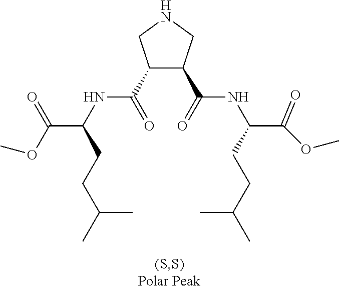

The title compound was prepared from the product of the previous step (0.303 g, 0.56 mmol) and HCl (4N in dioxane, 6.40 mL, 46.0 mmol) as described in Example 84, step b. Yield 0.29 g, 108%. Ion(s) found by LCMS: [M+H]+=442.4 Step c.

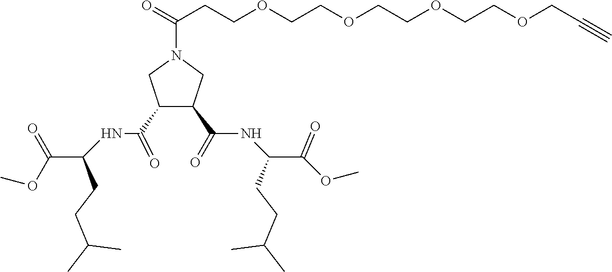 [SMILES: C#CCOCCOCCOCCOCCC(=O)N1C[C@@H](C(=O)N[C@@H](CCC(C)C)C(=O)OC)[C@H](C(=O)N[C@@H](CCC(C)C)C(=O)OC)C1]

The title compound was prepared from the product of the previous (0.29 g, 0.657 mmol) and propargyl-PEG4-acid (0.188 g, 0.722 mmol) as described in Example 84, step c. Yield 0.133 g, 30%. Ion(s) found by LCMS: (pos) (M+H)+=684.4, and (neg) (M−H)−=682.3.

### Step d

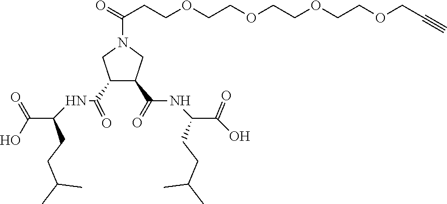 [SMILES: C#CCOCCOCCOCCOCCC(=O)N1C[C@@H](C(=O)N[C@@H](CCC(C)C)C(=O)O)[C@H](C(=O)N[C@@H](CCC(C)C)C(=O)O)C1]

The title compound was prepared from the bis-methyl esters product of the previous step (0.133 g, 0.194 mmol) and lithium hydroxide (0.014 g, 0.582 mmol) as described by the Example 84, step d. Yield 0.12 g, 94%. Ion(s) found by LCMS: (pos) (M+H)+=658.3 and (neg) (M−H)+=655.3

### Step e

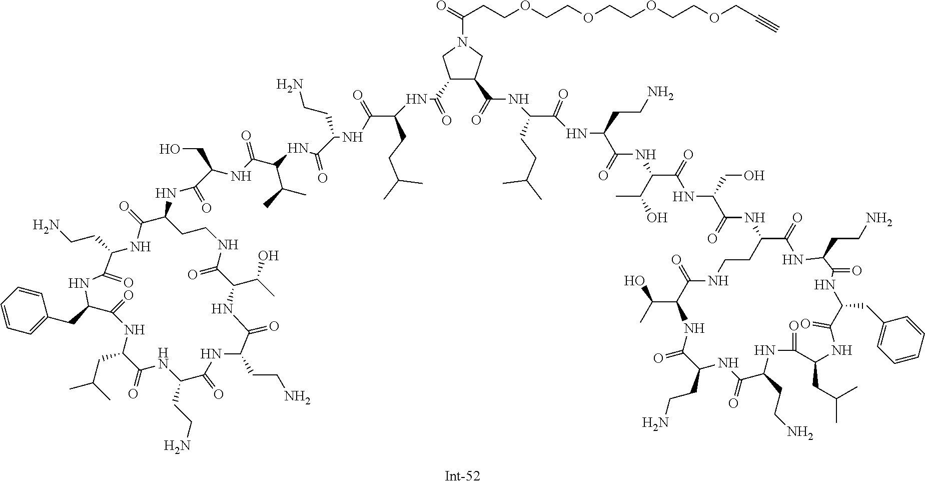

The title compound Int-52 was prepared from the di-acid product of the previous step (0.06 g, 0.092 mmol), and the product described in Example 6, Int-5d (0.292 g, 0.201 mmol) using the procedure described in Example 84, step e. Yield 0.135 g as TFA salt, 41%, 2 steps. Ion(s) found by LCMS: [(M+5H)/5]+=544.9, [(M+6H)/6]+=454.3

### Example 88—Synthesis of Int-53

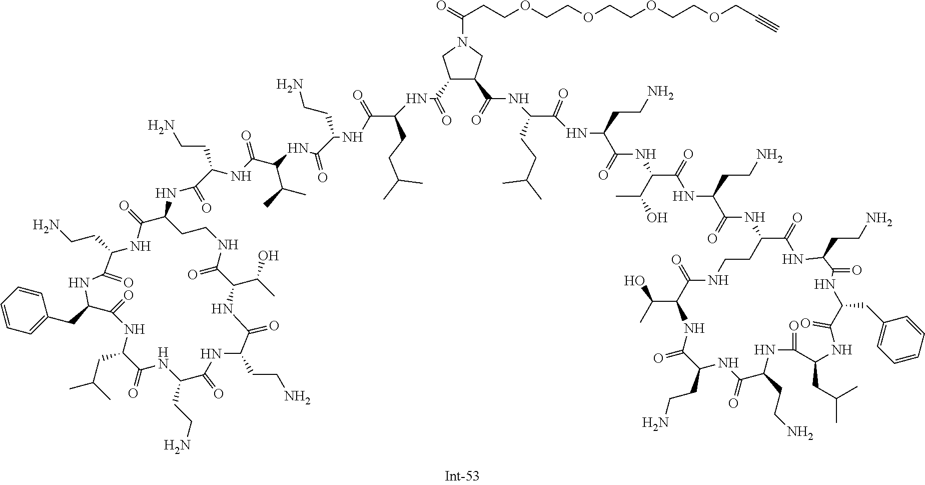

The title compound Int-53 was prepared from the di-acid product described in Example 87, step d (0.06 g, 0.092 mmol) and Int-7a (0.315 g, 0.212 mmol, Example 8, step a) using the procedure described in Example 84, step e. Yield 0.116 g as TFA salt, 35%, 2 steps. Ion(s) found by LCMS: [(M+4H)/4]+=687.2, [(M+5H)/5]+=550.1, [(M+6H)/6]+=458.6.

### Example 89—Synthesis of Int-54

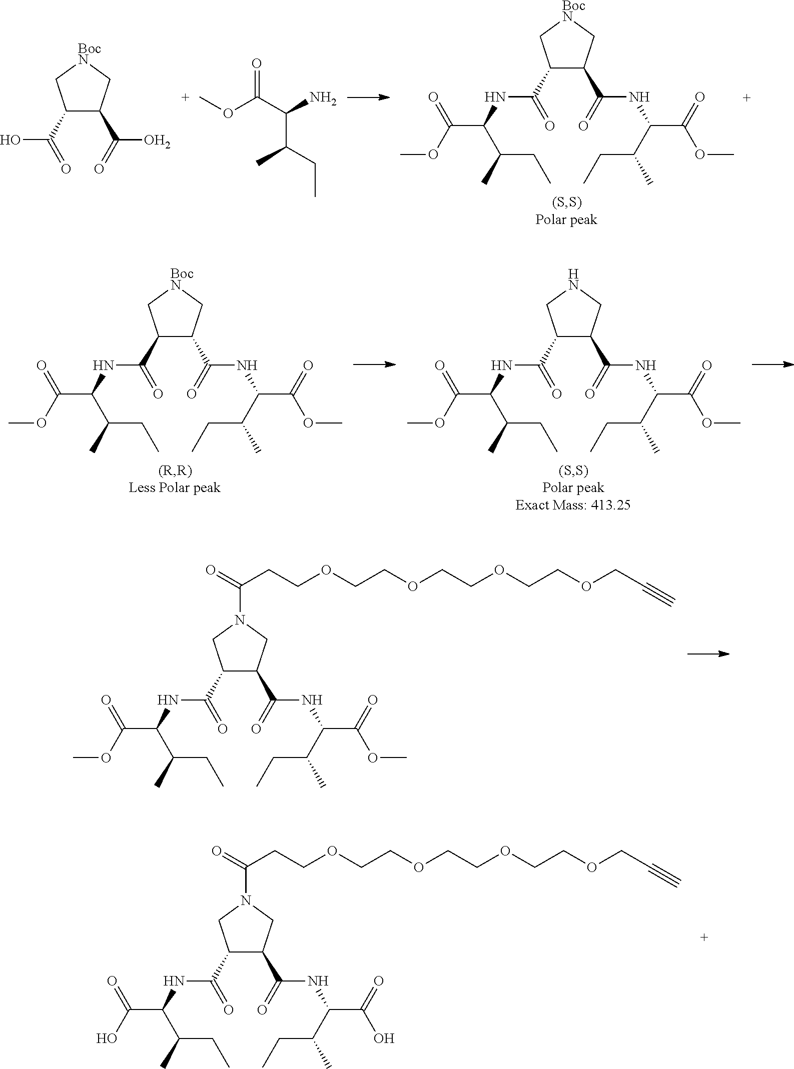

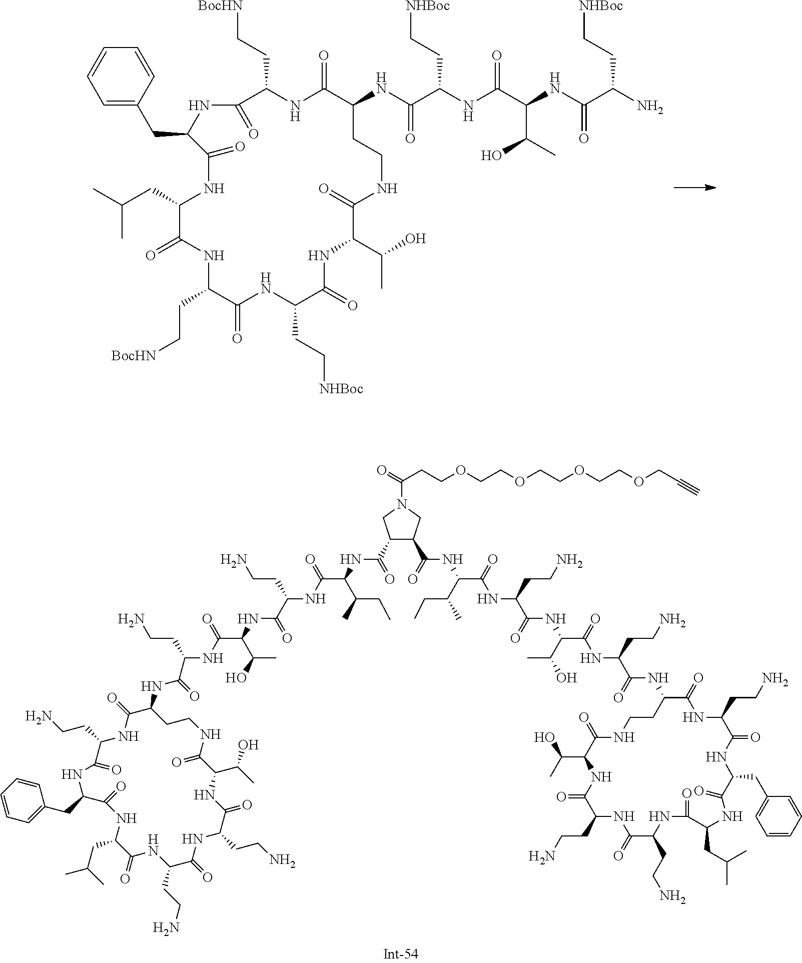

### Step a

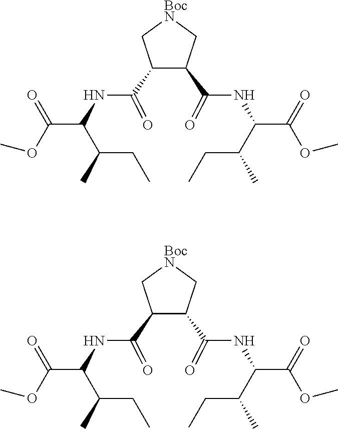

The title compound was prepared from methyl (2S,3R)-2-amino-3-methylpentanoate (0.242 g, 1.66 mmol) and racemic-trans1-(tert-butoxycarbonyl)pyrrolidine-3,4-dicarboxylic acid (0.210 g, 0.811 mmol) prepared analogously to Example 84, step a. Yield 0.121 g of polar isomer, 29%. Ion(s) found by LCMS: (M+H)+=514.2. Note: Only the polar isomer was taken forward to the next steps.

### Step b

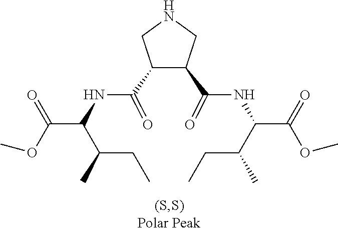

The title compound was prepared from the product of the previous step (0.121 g, 0.235 mmol) and HCl (4M in Dioxane, 0.59 mL, 2.35 mmol) as described in the procedure for Example 84, step b. Yield 0.122 g, 116%. Ion(s) found by LCMS: (M+H)+=414.2

### Step c

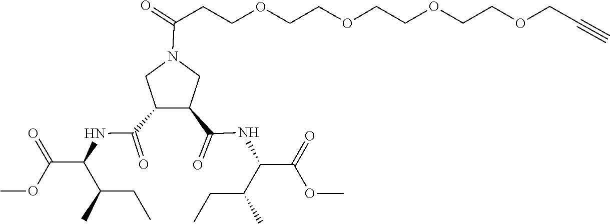 [SMILES: C#CCOCCOCCOCCOCCC(=O)N1C[C@@H](C(=O)N[C@H](C(=O)OC)[C@H](C)CC)[C@H](C(=O)N[C@H](C(=O)OC)[C@H](C)CC)C1]

The title compound was prepared from the product of the previous step (0.118 g, 0.261 mmol) and propargyl-PEG4-acid (0.188 g, 0.287 mmol) as described in Example 84, step c. Yield 0.075 g, 44%. Ion(s) found by LCMS: (pos) (M+H)+=656.4. (M+Na)+=679.3 and (neg) (M−H)+=654.2

### Step d

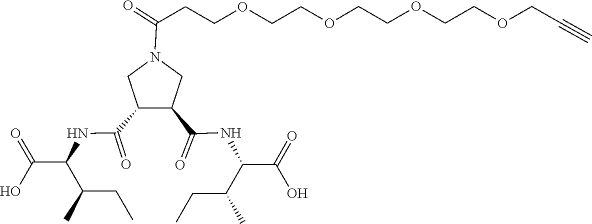 [SMILES: C#CCOCCOCCOCCOCCC(=O)N1C[C@@H](C(=O)N[C@H](C(=O)O)[C@H](C)CC)[C@H](C(=O)N[C@H](C(=O)O)[C@H](C)CC)C1]

The title compound was prepared from bis-methyl ester product in the previous step (0.075 g, 0.114 mmol) and lithium hydroxide (0.008 g, 0.34 mmol) as described by a procedure of Example 84 Step d. Yield 0.056 g, 78%. Ion(s) found by LCMS: (M+H)+=628.3

### Step e

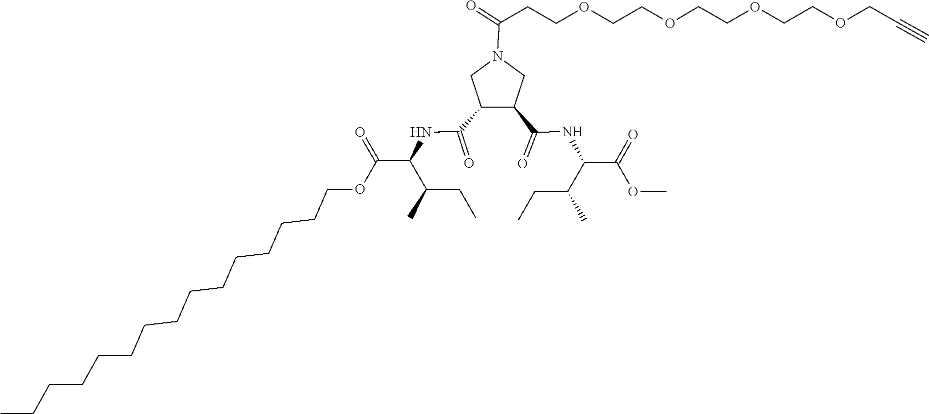 [SMILES: C#CCOCCOCCOCCOCCC(=O)N1C[C@@H](C(=O)N[C@H](C(=O)OC)[C@H](C)CC)[C@H](C(=O)N[C@H](C(=O)OCCCCCCCCCCCCCCC)[C@H](C)CC)C1]

The title compound Int-54 was prepared from the di-acid product of the previous step (0.056 g, 0.088 mmol) and Int-7a (0.305 g, 0.195 mmol, Example 8, step a) using the procedure from Example 84, step e. Yield of 0.0326 g as TFA salt, 10%. Ion(s) found LCMS: [(M+5H)/5]+=544.7, [(M+6H)/6]+=454.3

### Example 90—Synthesis of Int-55

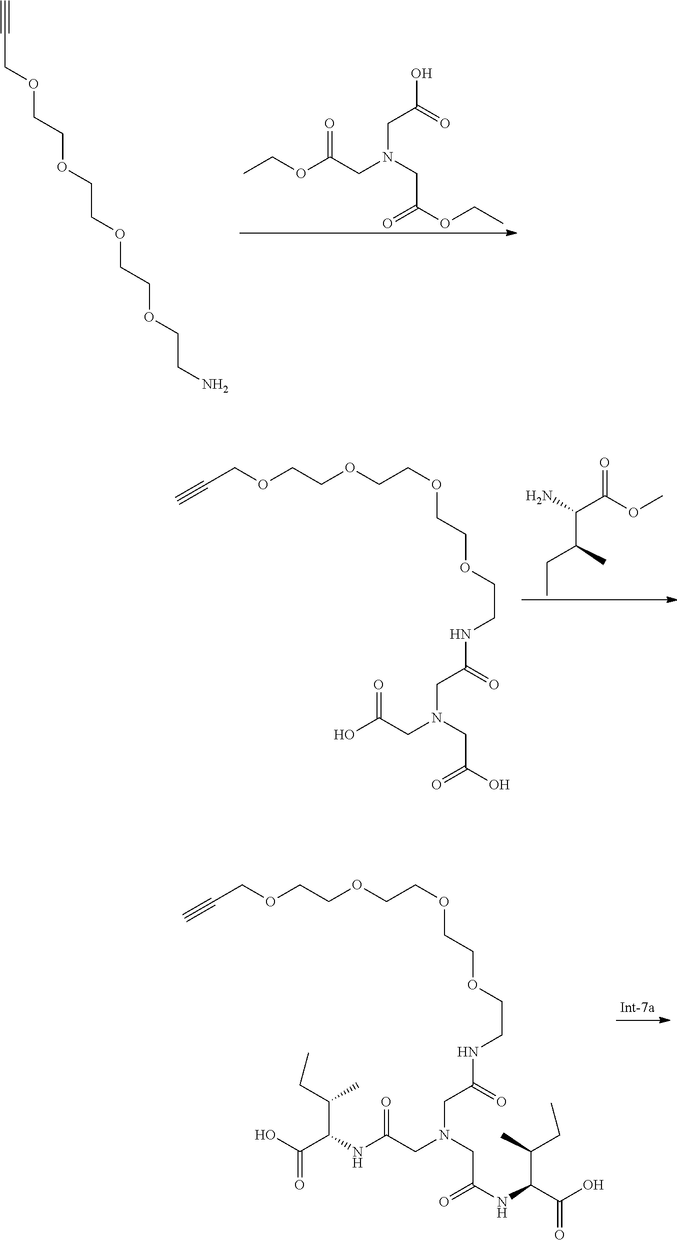

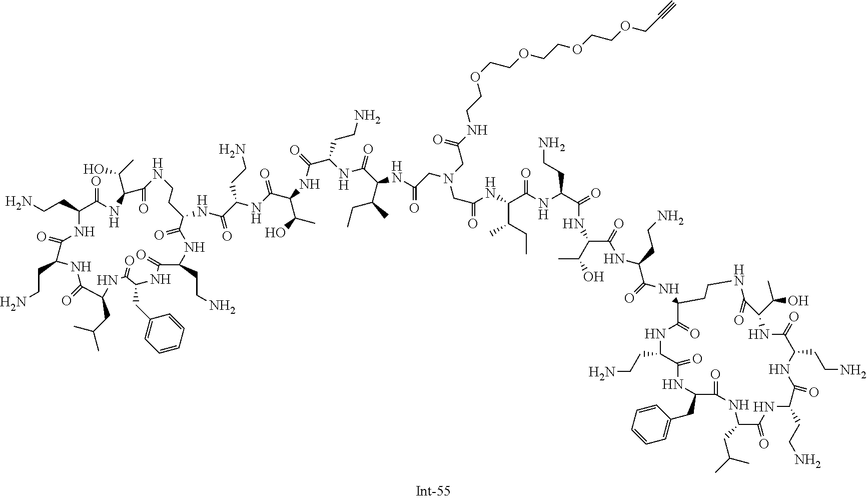 [SMILES: C#CCOCCOCCOCCOCCNC(=O)CN(CC(=O)N[C@H](C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O)[C@@H](C)CC)CC(=O)N[C@H](C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O)[C@@H](C)CC]

### Step a

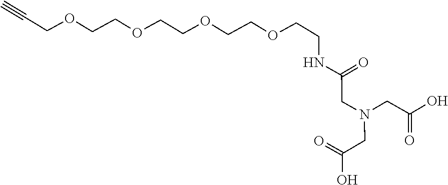 [SMILES: C#CCOCCOCCOCCOCCNC(=O)CN(CC(=O)O)CC(=O)O]

EDC (0.77 g, 4.0 mmol) was added to a stirring mixture of diester (1.0 g, 4.0 mmol), HOBt (0.62 g, 4.0 mmol), and propargyl-peg4-amine (0.94 g, 4.0 mmol) in DMF (8 mL) and stirred for 2 hours. The mixture was reduced by half on a rotary evaporator and then purified by RPLC using an Isco Combiflash liquid chromatograph eluted with 5% to 95% acetonitrile and water using 0.1% TFA modifier. The pure fractions were pooled and lyophilized to afford the di-ester as a clear oil. Positive ions found: by LCMS (M+H⁺)=461.2.

The di-ester was stirred in a 1:1:2 mixture (5 mL) of THF/MeOH/DI water containing LiOH (0.39 g, 16.2 mmol) for 1 hour. The mixture was acidified with glacial acetic acid (˜0.7 mL) and concentrated to half the volume on the rotary evaporator. The di-acid was purified RPLC using an Isco Combiflash liquid chromatograph eluted with 10% to 95% acetonitrile and water using 0.1% TFA modifier. The pure fractions were pooled and lyophilized to afford the di-acid as a viscous clear oil. Yield 953 mg, 59.8%, 2 steps. Positive ions found by LCMS (M+H⁺)=405.2.

### Step b

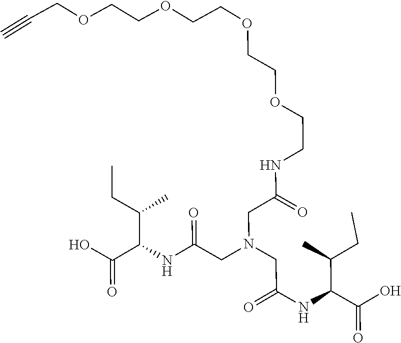 [SMILES: C#CCOCCOCCOCCOCCNC(=O)CN(CC(=O)N[C@H](C(=O)O)[C@@H](C)CC)CC(=O)N[C@H](C(=O)O)[C@@H](C)CC]

EDC (0.12 g, 0.65 mmol) was added to a stirring mixture of Isoleucine-methyl ester hydrochloride salt (0.17 g, 0.93 mmol), HOBt (0.099 g, 0.65 mmol), intermediate from the previous step (0.13 g, 0.31 mmol), and triethylamine (0.097 g, 0.96 mmol) in DMF (1.5 mL) and the reaction was stirred for 12 hours. The mixture was applied directly to RPLC using an Isco Combiflash liquid chromatograph eluted with 20% to 95% acetonitrile and water using 0.1% TFA modifier. The pure fractions were pooled and lyophilized to afford the 200 mg of the di-ester as a white solid. Positive ions found: by LCMS (M+H⁺)=659.4. The di-acid was hydrolyzed by stirring in a 1:1:2 mixture THF/MeOH/DI water (8 mL) containing LiOH (0.029 g, 1.2 mmol) for 30 minutes at ambient temperature. Glacial acetic acid (˜1 mL) was added and the volume was reduced by 70% on the rotary evaporator. The residue was purified by RPLC using an Isco Combiflash liquid chromatograph eluted with 5% to 85% acetonitrile and water using 0.1% TFA modifier. The pure fractions were pooled and lyophilized to afford di-acid as a clear oil. Yield 73%, 2 steps. Positive ions found: by LCMS (M+H⁺)=631.4.

### Step c

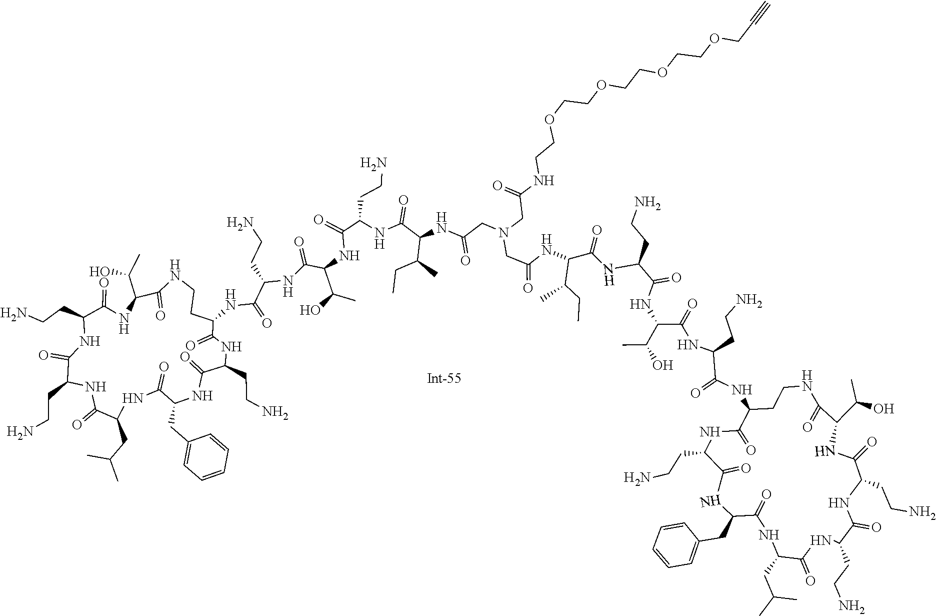

EDC (89 mg, 0.47 mol) was added to a stirring mixture of intermediate from previous step (140 mg, 0.22 mmol), Int-7a (760 mg, 0.49 mmol), and HOBt (71 mg, 0.47 mmol) in 1.5 mL of DMF. The reaction was stirred for 12 hours at ambient temperature. The mixture was then applied directly to RPLC using an Isco Combiflash liquid chromatograph eluted with 30% to 100% methanol and water using 0.1% TFA modifier. The fractions containing the positive ion mass observed by LCMS [(M−(3Boc)+3H⁺)/3]=1141.2 were pooled and concentrated. The Boc-protected dimer was stirred in a 1/1 mixture of TFA/DCM (10 mL) at ambient temperature for 30 minutes. The solvent was removed by rotary evaporator and dried under high vacuum. The crude deprotected dimer was purified by RPLC using an Isco Combiflash liquid chromatograph eluted with 0% to 90% acetonitrile and water using 0.1% TFA modifier. The pure fractions were pooled and lyophilized to afford desired product as a white solid deca-TFA salt. Yield of Int-55 was 405 mg, 67%, 2 steps. Positive mass ions were found by LCMS (M+4H)/4=680.8, LCMS (M+5H⁺)/5=544.6, and LCMS (M+6H⁺)/6=454.2.

### Example 91—Synthesis of Int-566

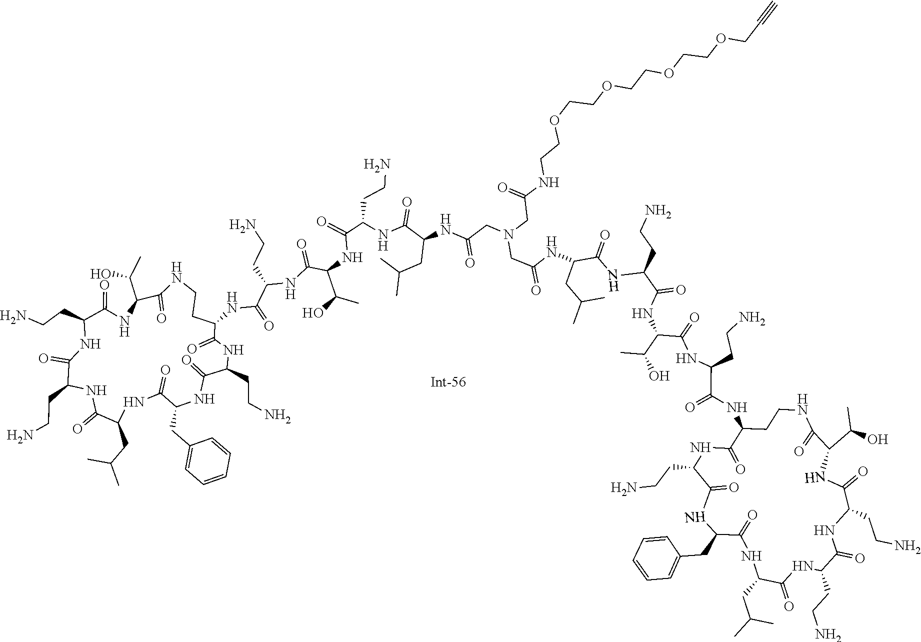

The title compound Int-56 was prepared analogously to Example 90, where L-leucine was substituted for L-isoleucine in step A of that example. Yield 430 mg, 66%, 2 steps. Positive ions found by LCMS: (M+4H⁺)/4=680.8, (M+5H⁺)/5=544.6, and (M+6H⁺)/6=454.2

### Example 92—Synthesis of Int-57

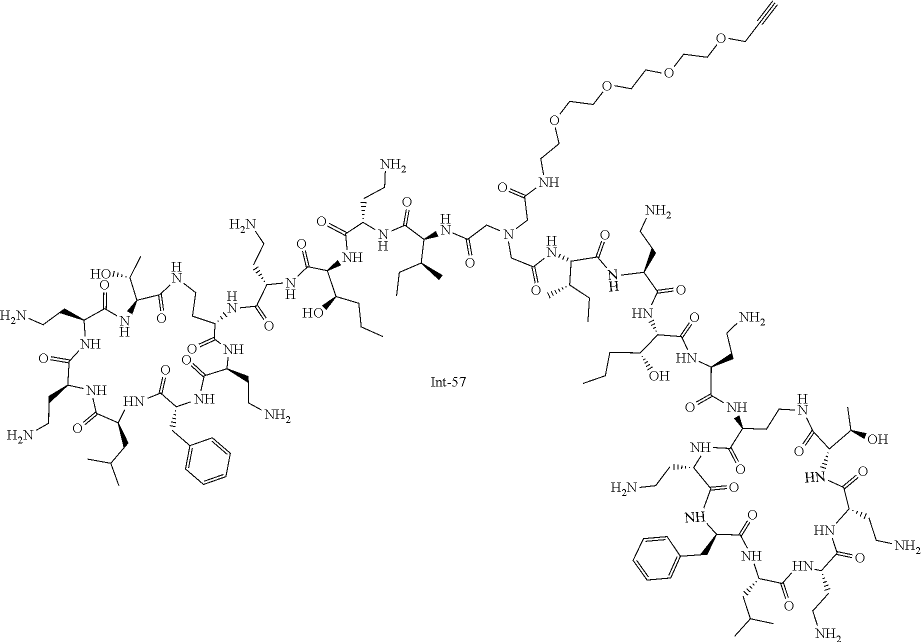

The title compound Int-57 was prepared analogously to Example 90 where Int-7a was substituted with Int-7a described in Example 67, step h and the di-acid intermediate described in step b of Example 90. Yield of 210 mg, 69%, 2 steps. Ions found by LCMS (M+3H⁺)/3=926.2, LCMS (M+4H⁺)/4=695.2, and LCMS (M+5H⁺)/5=556.4.

### Example 93—Synthesis of Int-58

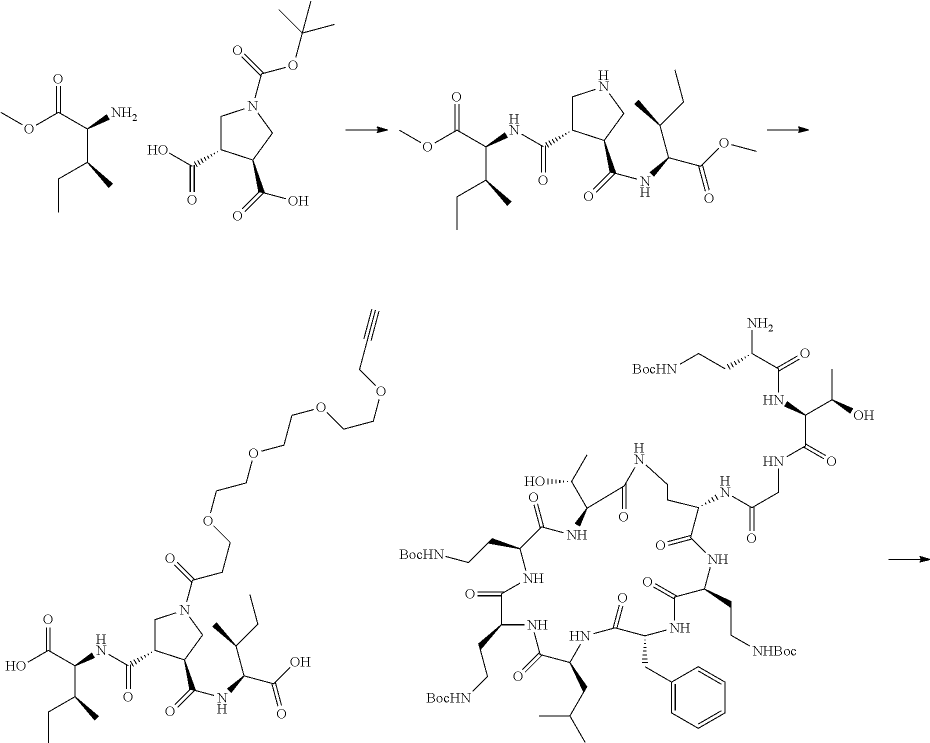

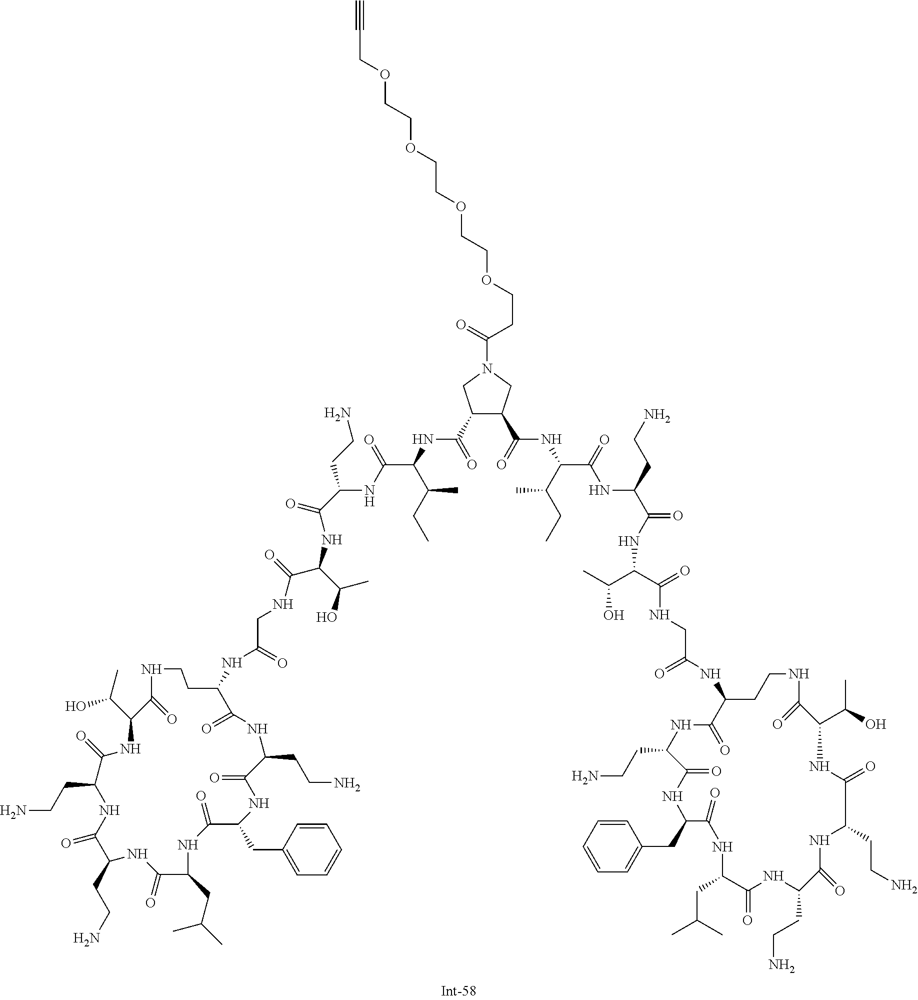 [SMILES: C#CCOCCOCCOCCOCCC(=O)N1C[C@@H](C(=O)N[C@H](C(=O)N[C@@H](CCN)C(=O)N[C@H](C(=O)NCC(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)[C@@H](C)CC)[C@H](C(=O)N[C@H](C(=O)N[C@@H](CCN)C(=O)N[C@H](C(=O)NCC(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)[C@@H](C)CC)C1]

### Step a

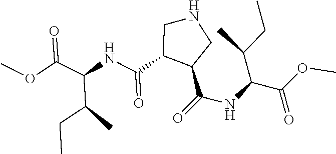

EDC (3.2 g, 17 mmol) was added to a stirring mixture of racemic-trans1-(tert-butoxycarbonyl)pyrrolidine-3,4-dicarboxylic acid (2 g, 7.7 mmol), Isoluecine methyl ester HCl salt (3.5 g, 19.3 mmol), HOBt (2.6 g, 17 mmol), and triethylamine (2 g, 20 mmol) in 15 mL of DMF. The reaction was stirred for 12 hours, diluted with aqueous 1N HCl (100 mL), extracted into ethyl acetate, dried over sodium sulfate and concentrated. The diastereomers were separated by RPLC using an Isco Combiflash liquid chromatograph eluted with 15% to 95% acetonitrile and water using 0.1% TFA modifier. The polar isomer was pooled and lyophilized to afford 1.2 g of the boc-protected ester. Yield, combined 59%.LCMS [M−(1boc)+H⁺)=414.2. The polar isomer boc-protected ester (1.2 g, 2.3 mmol)) was stirred in 30 mL of 4M HCl (g) in dioxane for 30 minutes. The solvent was removed by rotary evaporator and dried under high vacuum to afford the di-ester-Ile-pyrolidine HCl salt as a white solid. Yield of 0.60 g, 75%. LCMS [M+H⁺)=414.2.

### Step b

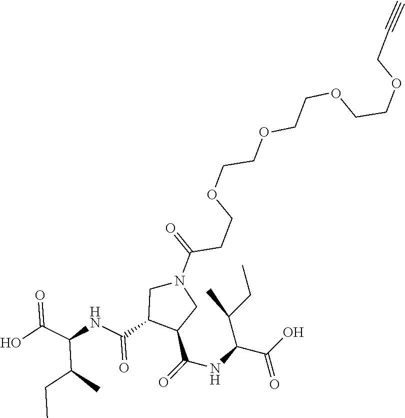 [SMILES: C#CCOCCOCCOCCOCCC(=O)N1C[C@@H](C(=O)N[C@H](C(=O)O)[C@@H](C)CC)[C@H](C(=O)N[C@H](C(=O)O)[C@@H](C)CC)C1]

HATU (389 mg, 0.87 mmol) was added to a stirring mixture of the trans-di-methyl ester-Ile-pyrolidine HCl salt (325 mg, 0.72 mmol), propargyl-peg4 acid (226 mg, 0.86 mmol), and triethylamine (291 mg, 2.9 mmol) and stirred at ambient temperature for 30 minutes. The mixture was applied directly to RPLC using an Isco Combiflash liquid chromatograph eluted with 20% to 95% acetonitrile and water using 0.1% TFA modifier. Fractions showing desired ions by LCMS (M+H⁺)=656.4 were pooled and concentrated. The residue was stirred in a 1:1:2 mixture of methanol/THF/DI water containing LiOH (52 mg, 2.2 mmol) at ambient temperature for 30 minutes. The reaction mixture was acidified with a few drops of acetic acid and the volume was reduced by half on the rotary evaporator. The mixture was purified by RPLC using an Isco Combiflash liquid chromatograph eluted with 10% to 95% acetonitrile and water using 0.1% TFA modifier. The pure fractions were pooled and lyophilized to afford the propargyl peg4 di-acid intermediate as a clear oil. Yield of 375 mg, 83%, 2 steps. Positive ions were found by LCMS [M+H⁺)=626.2.

### Step c

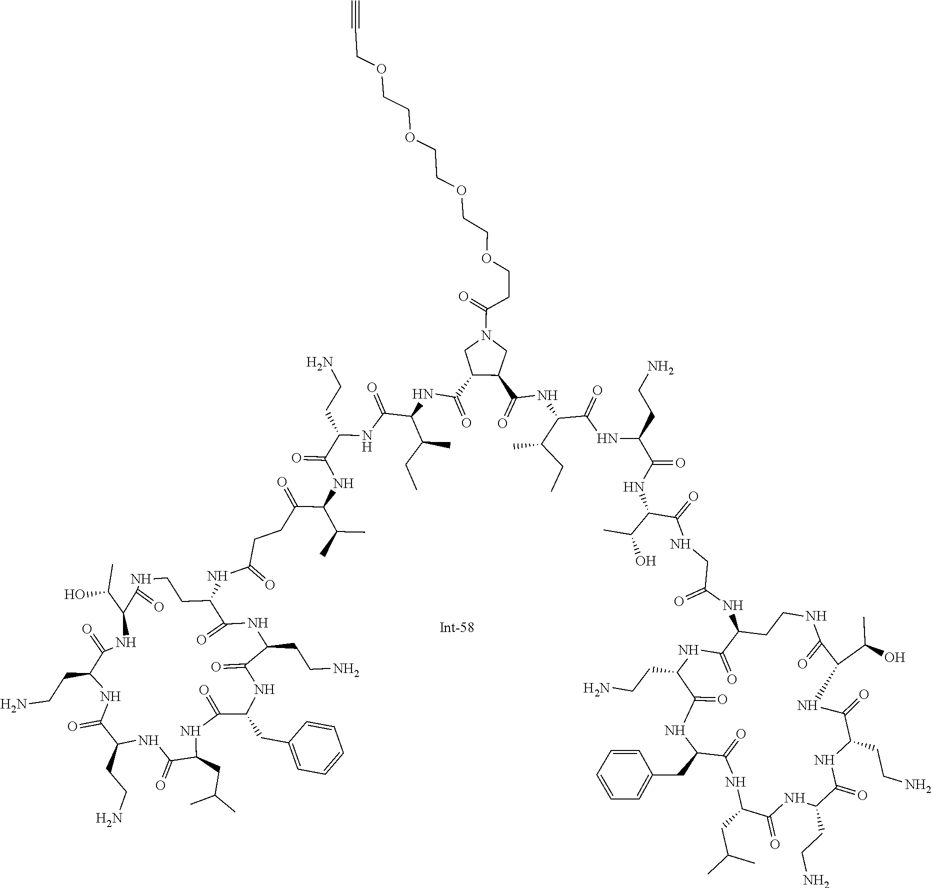

The title compound Int-58 was prepared analogously to Example 90 where Int-7a was substituted with tetra Boc PMBH-Gly-Thr-Dab (described in Example 100, step c), and the intermediate described in step b of this example. Yield; 54%, 2 steps. Positive mass ions were found by LCMS (M+3H⁺)/3=877.8, LCMS (M+4H⁺)/4=658.8, LCMS (M+5H)/5=527.4, and LCMS (M+6H⁺)/6=439.8.

### Example 94—Synthesis of Int-59

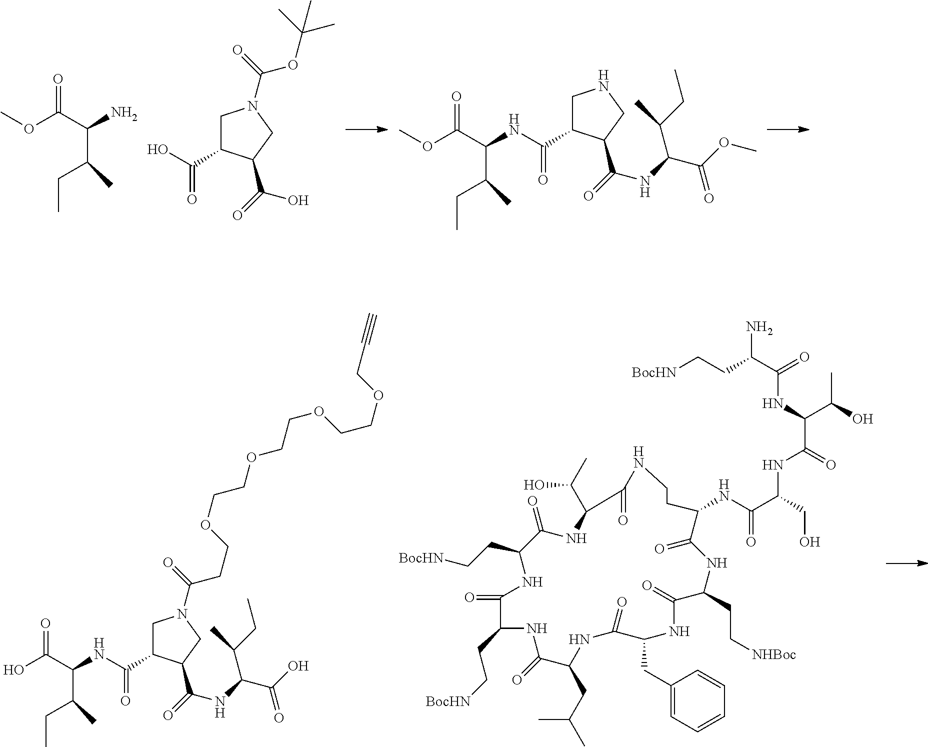

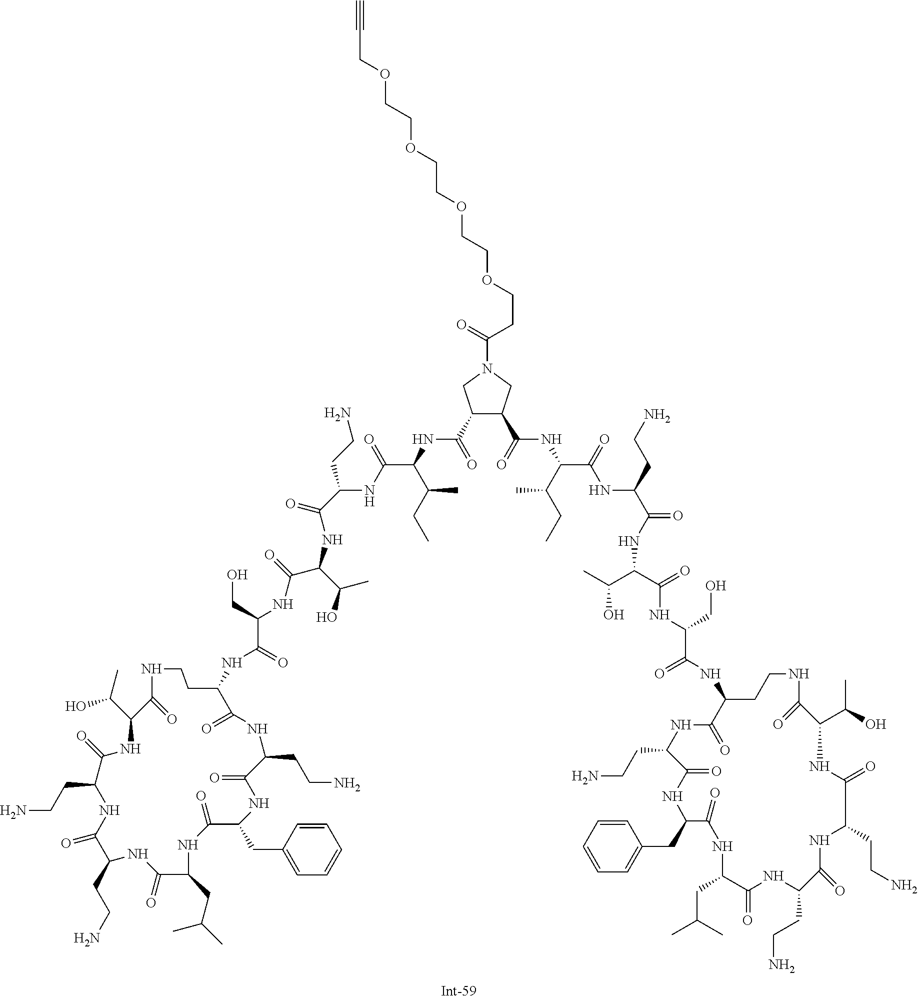 [SMILES: C#CCOCCOCCOCCOCCC(=O)N1C[C@@H](C(=O)N[C@H](C(=O)N[C@@H](CCN)C(=O)N[C@H](C(=O)N[C@H](CO)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)[C@@H](C)CC)[C@H](C(=O)N[C@H](C(=O)N[C@@H](CCN)C(=O)N[C@H](C(=O)N[C@H](CO)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)[C@@H](C)CC)C1]

The title compound Int-59 was prepared analogously to Example 90 step c from the intermediate from Example 93, step b and Int-5d Example 6. Yield; 44%, 2 steps. Positive mass ions were found by LCMS (M+3H⁺)/3=898.0, LCMS (M+4H⁺)/4=673.8, LCMS (M+5H⁺)/5=539.4, and LCMS (M+6H⁺)/6=449.8.

### Example 95—Synthesis of Int-60

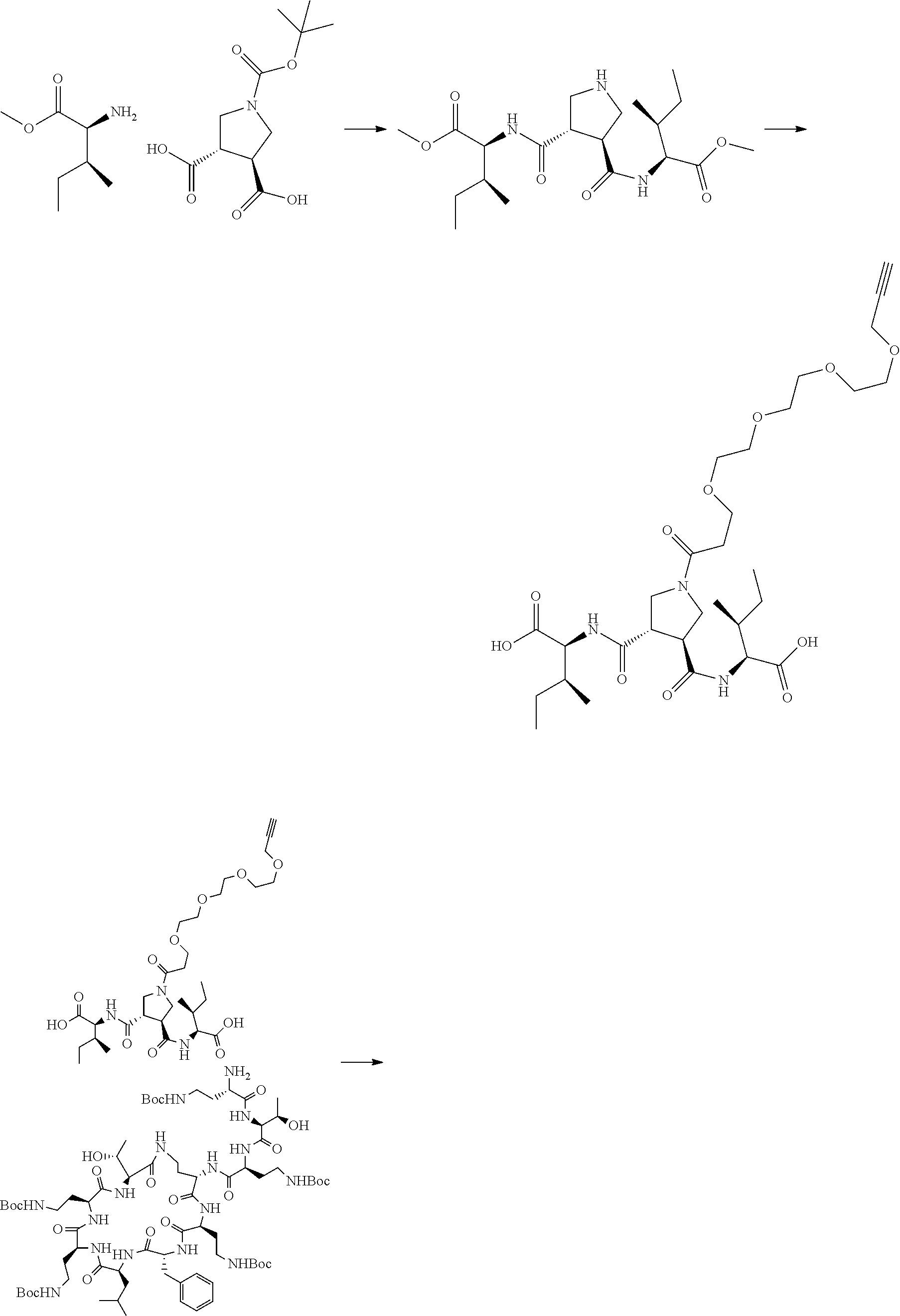

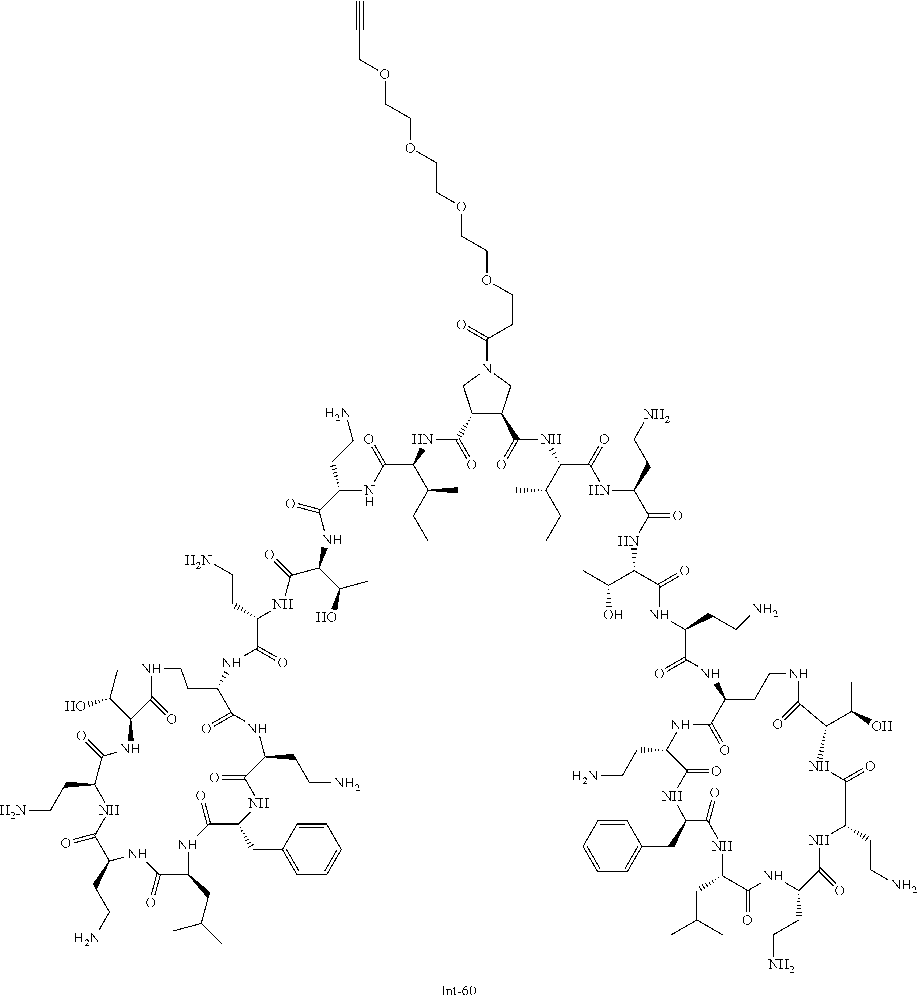 [SMILES: C#CCOCCOCCOCCOCCC(=O)N1C[C@@H](C(=O)N[C@H](C(=O)N[C@@H](CCN)C(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)[C@@H](C)CC)[C@H](C(=O)N[C@H](C(=O)N[C@@H](CCN)C(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)[C@@H](C)CC)C1]

The title compound Int-60 was prepared analogously to Example 90, step c from Int-7a (Example 2, step a) and the intermediate from Example 93, step b. Yield of 195 mg, 49%, 2 steps. Positive mass ions were found by LCMS (M+3H′)/3=906.4, LCMS (M+4H′)/4=680.3, LCMS (M+5H′)/5=544.5.

### Example 96—Synthesis of Int-61

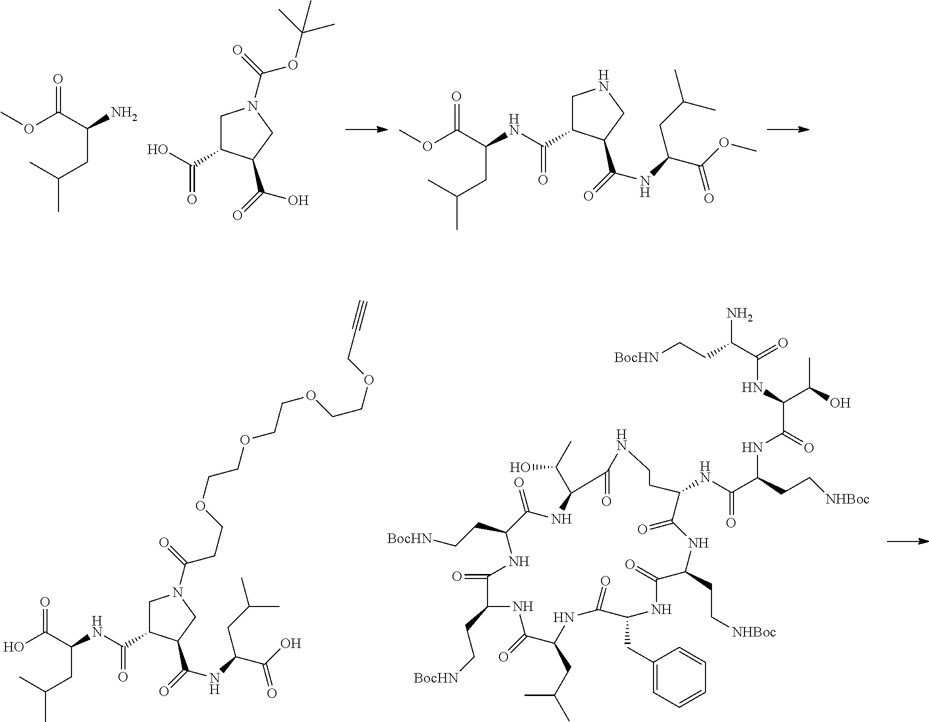

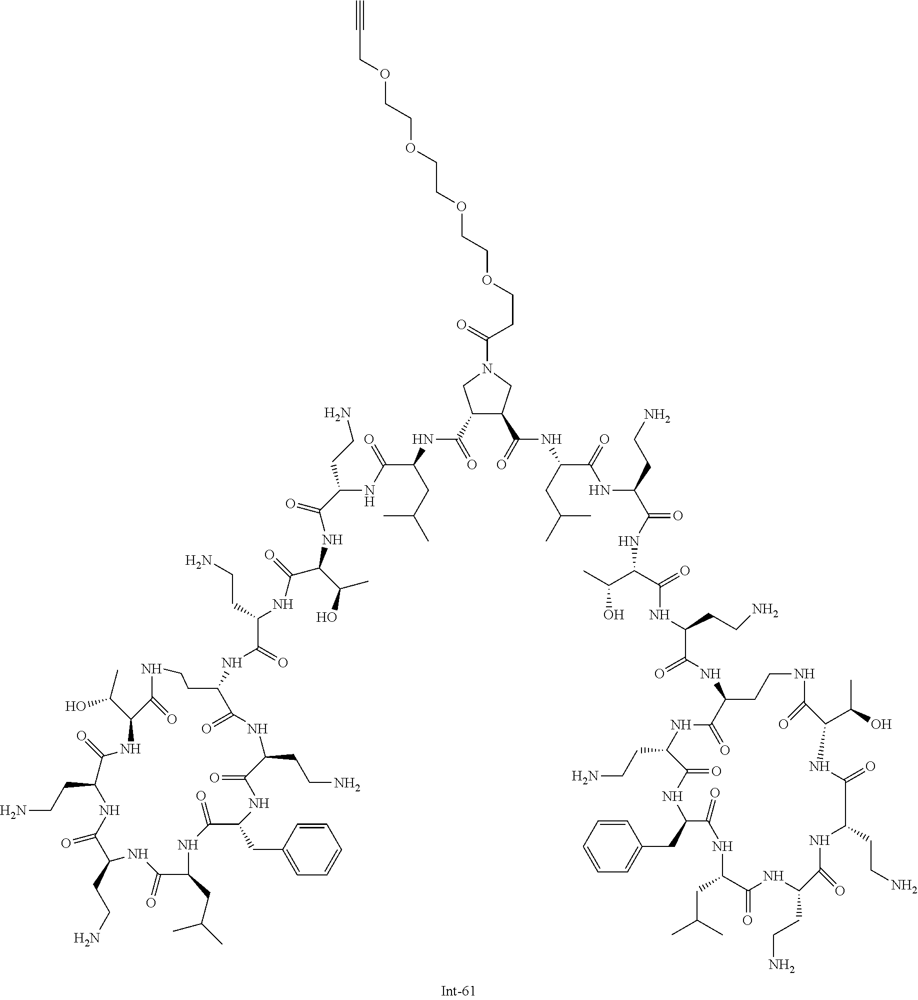 [SMILES: C#CCOCCOCCOCCOCCC(=O)N1C[C@@H](C(=O)N[C@@H](CC(C)C)C(=O)N[C@@H](CCN)C(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)[C@H](C(=O)N[C@@H](CC(C)C)C(=O)N[C@@H](CCN)C(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)C1]

### Step a

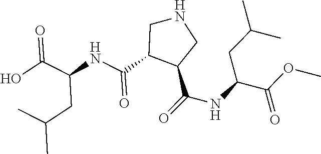 [SMILES: COC(=O)[C@H](CC(C)C)NC(=O)[C@@H]1CNC[C@H]1C(=O)N[C@@H](CC(C)C)C(=O)O]

The dimethyl ester was prepared analogously to intermediate Example 93, step a substituting L-leucine for L-isoleucine. Yield of 0.85 g, 51%, 2 steps. LCMS (M+H)+=414.2.

### Step b

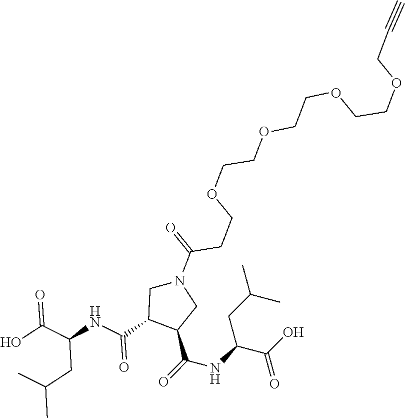 [SMILES: C#CCOCCOCCOCCOCCC(=O)N1C[C@@H](C(=O)N[C@@H](CC(C)C)C(=O)O)[C@H](C(=O)N[C@@H](CC(C)C)C(=O)O)C1]

The diacid was prepared analogously to Example 93, step b. Yield of 0.82 g, 77%, 2 steps. Negative Ion found by LCMS [M−H]−=626.2.

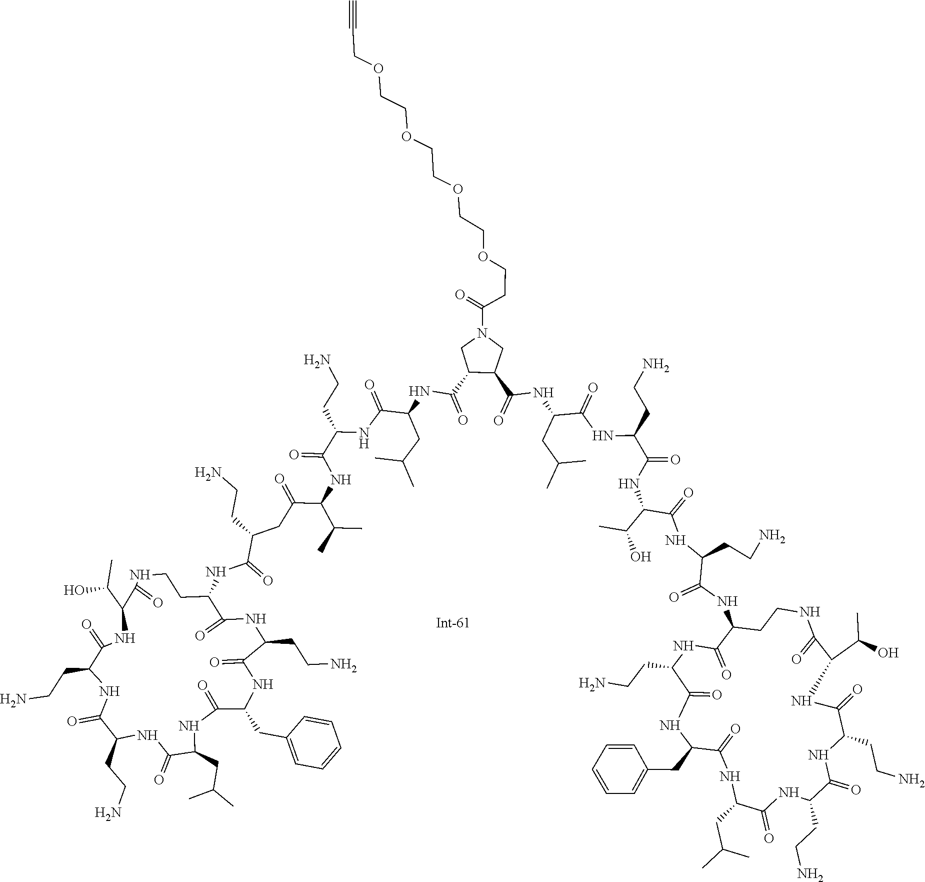

The title compound Int-61 was prepared analogously to Example 90, step c from the previous intermediate and Int-7a. Yield of 179 mg, 39%, 2 steps. Positive mass ions were found by LCMS (M+3H⁺)/3=906.6, LCMS (M+4H⁺)/4=680.3, LCMS (M+5H⁺)/5=544.5.

### Example 97—Synthesis of Int-62

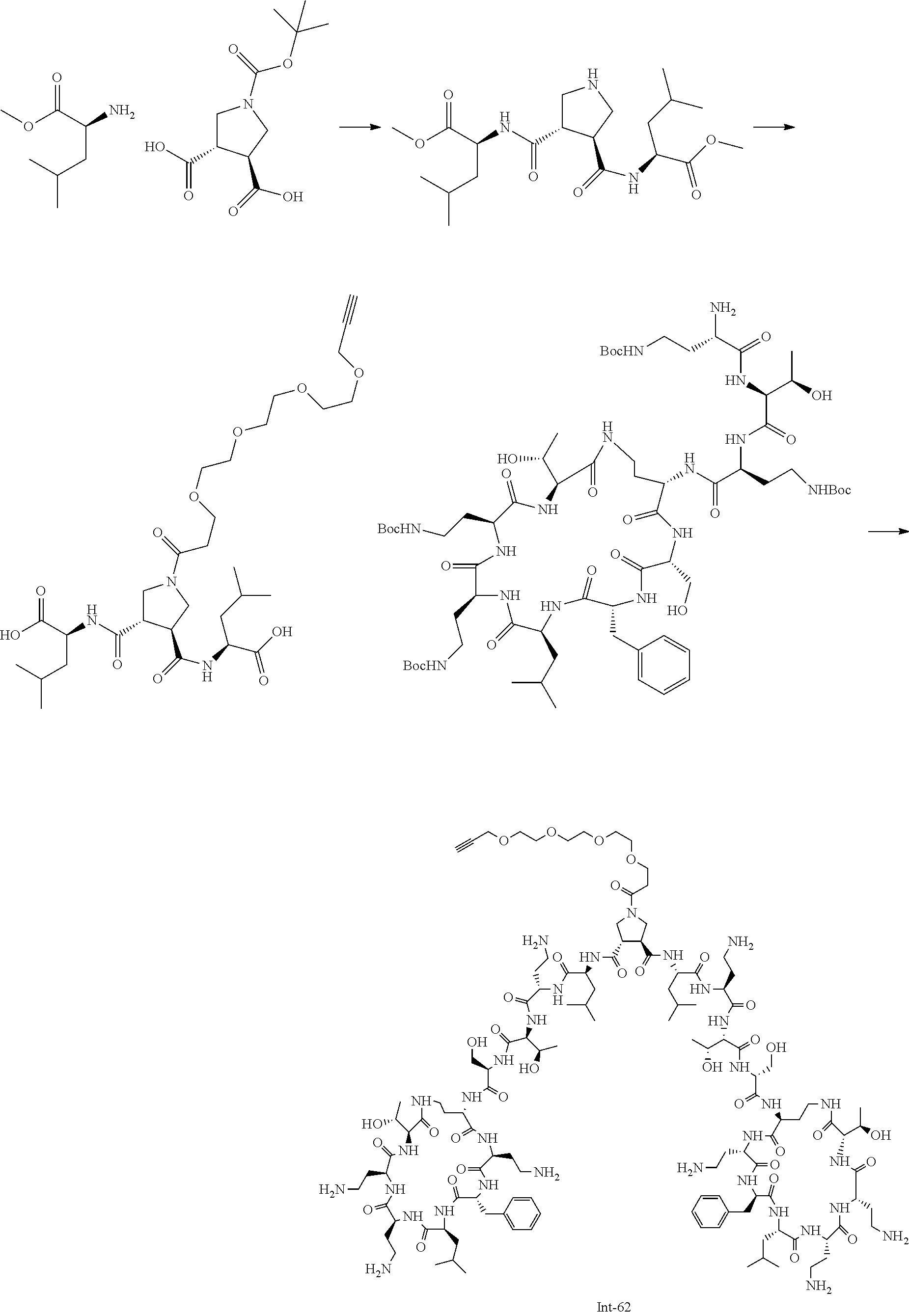

The title compound Int-62 was prepared analogously to Example 90, step c from the intermediates described in Example 96 step b, and Example 6, Int-5d. Yield of 225 mg 36%, 2 steps. Positive mass ions were found by LCMS (M+3H⁺)/3=897.8. LCMS (M+4H⁺)/4=674.0, LCMS (M+5H⁺)/5=539.4.

### Example 98—Synthesis of Int-63

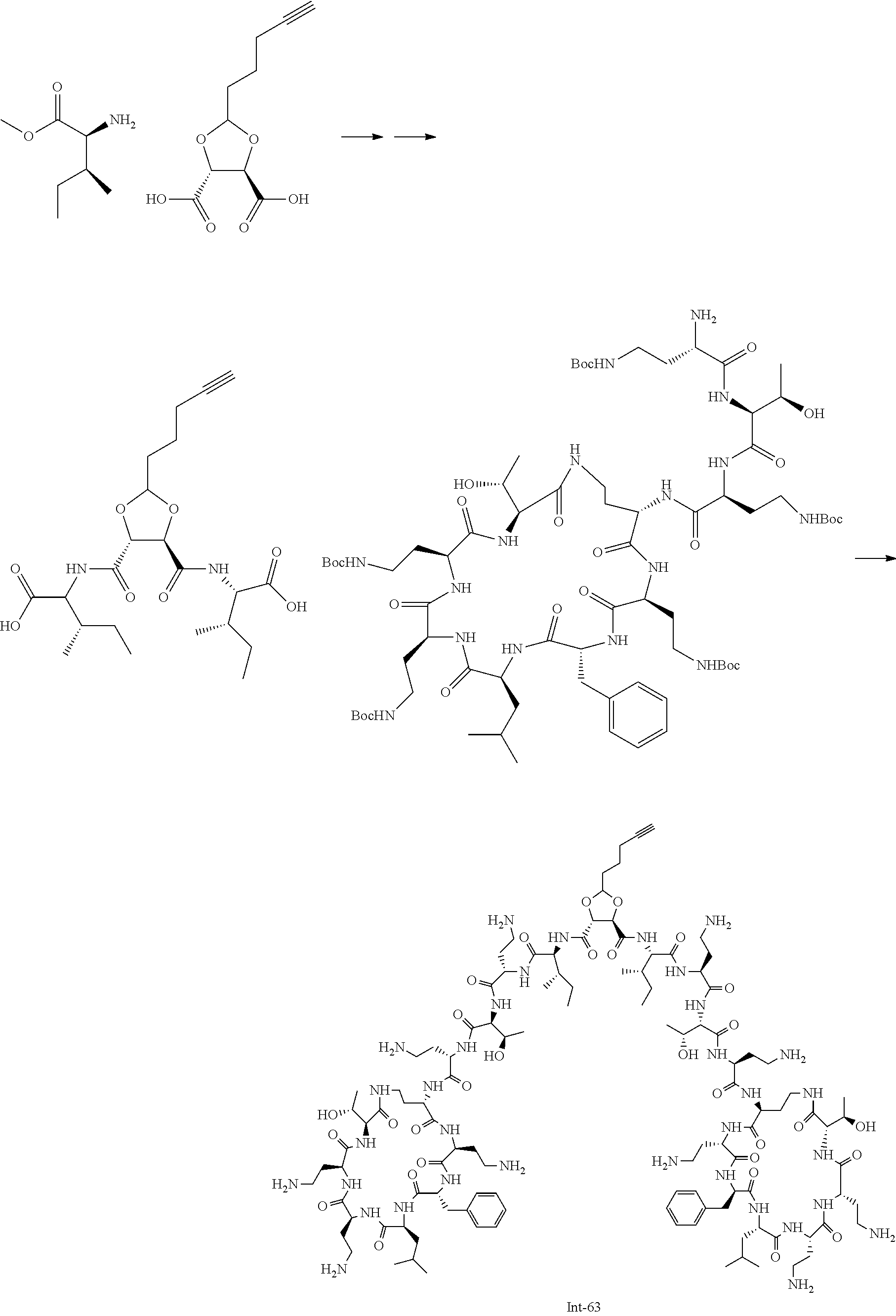

### Step a

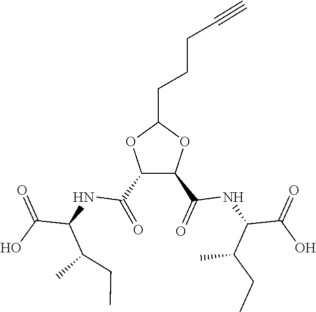 [SMILES: C#CCCCC1O[C@@H](C(=O)N[C@H](C(=O)O)[C@@H](C)CC)[C@H](C(=O)N[C@H](C(=O)O)[C@@H](C)CC)O1]

EDC (175 mg, 0.92 mmol) was added to a stirring mixture of di acid (synthesis described in Example 60, step b, 100 mg, 0.43 mmol), isoluecine methyl ester hydrochloride (239 mg, 1.3 mmol), HOBt (141 mg, 0.92 mmol), and triethylamine (132 mg, 1.3 mmol) in 1.5 mL of DMF. The reaction was stirred at ambient temperature for 12 hours then purified by RPLC using an Isco Combiflash liquid chromatograph eluted with 10% to 95% acetonitrile and water using 0.1% TFA modifier. Pure fractions were pooled and concentrated to afford an oil; ion(s) found by LCMS (M+H⁻)=483.3. The oil was taken up in a 1:1:2 mixture of methanol/THF/DI water (5 mL) containing LiOH (31 mg, 1.3 mmol) and stirred at ambient temperature for 30 minutes. The mixture was acidified (pH˜0.5) with a few drops of glacial acetic acid and concentrated to half volume. The mixture was purified by RPLC using an Isco Combiflash liquid chromatograph eluted with 5% to 80% acetonitrile and water using 0.1% TFA modifier. Pure fractions were pooled and lyophilized to afford the di-acid intermediate as a clear oil. Yield of 147 mg, 69%, 2 steps. Positive Ion found by LCMS (M+H⁺)=455.3.

### Step b

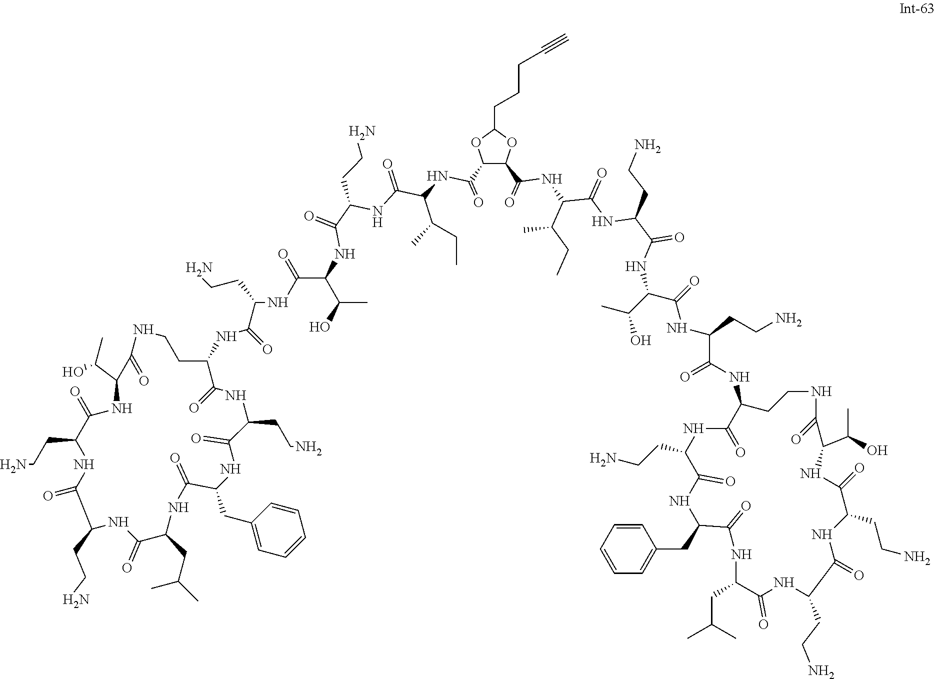

The title compound Int-63 was prepared analogously from Int-7a and the previous intermediate from this example. Yield, 260 mg, 77%, 2 steps. Positive mass ions were found by LCMS (M+3H⁺)/3=848.8, LCMS (M+4H⁺)/4=637.0, LCMS (M+5H⁺)/5=509.9.

### Example 99: Synthesis of Int-64

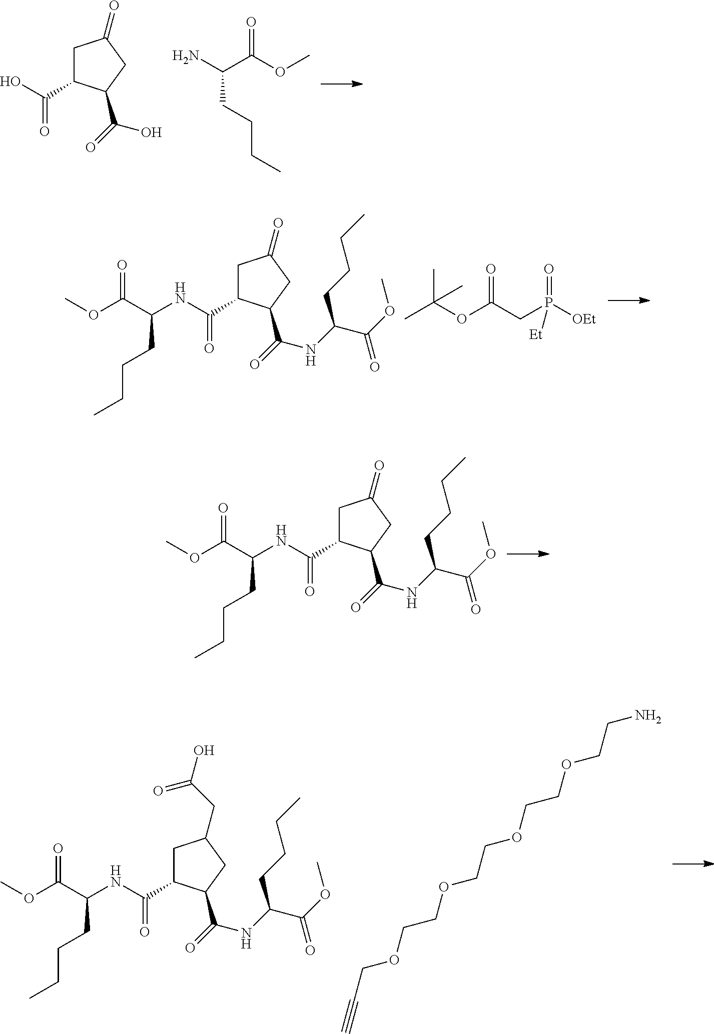

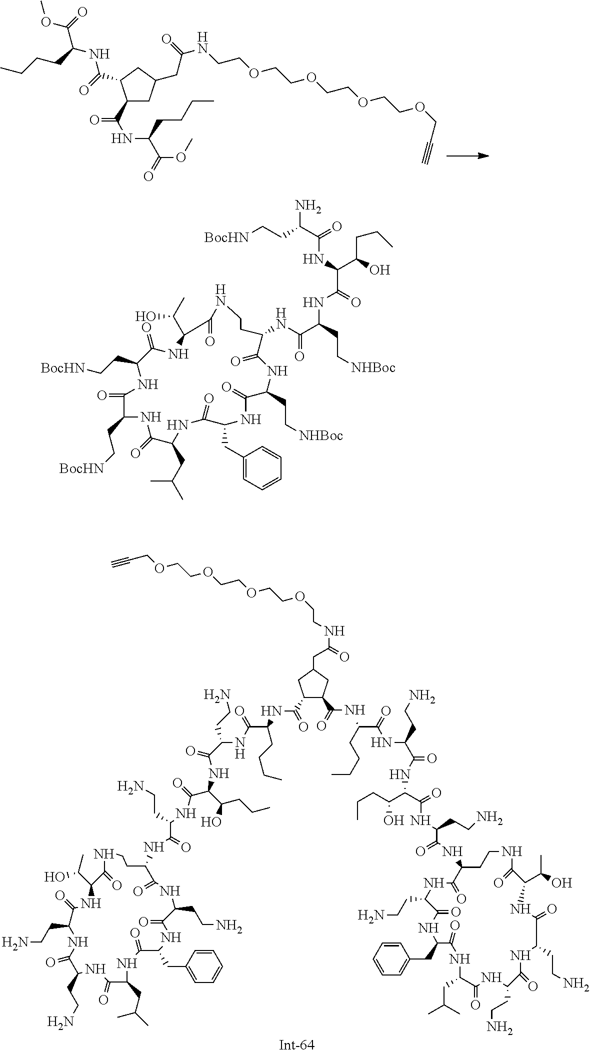

### Step a

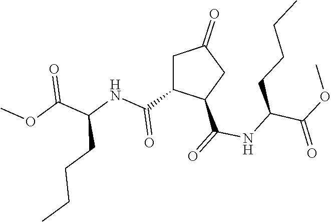 [SMILES: CCCC[C@H](NC(=O)[C@@H]1CC(=O)C[C@H]1C(=O)N[C@@H](CCCC)C(=O)OC)C(=O)OC]

EDC (2.04 g, 10.7 mmol) was added to a stirring mixture of racemic-4-oxocyclopentane-1,2-dicarboxylic acid (0.8 g, 4.6 mmol), HOBt (1.6 g, 10.7 mmol), and triethylamine (1.5 g, 14.4 mmol) in DMF (12 mL) and the reaction was stirred for 12 hours. The mixture was diluted with ethyl acetate (75 mL) washed with 1N HCl (100 mL). The aqueous phase was back extracted with ethyl acetate (2×, 50 mL). The combined organics were washed with brine (100 mL), dried over sodium sulfate and concentrated. The residue was purified by ISCO Combi Flash reversed phase HPLC (acetonitrile in DI water with 0.1% TFA as modifier, 10-95% acetonitrile, 40 minute gradient. The 2 diastereomers were separated and the more polar diastereomer (by HPLC) was taken forward in subsequent reactions. Yield of 1.7 g, 85%. LCMS (M+H⁺)=427.8. The shown stereochemistry was assigned by X-ray crystallography.

### Step b

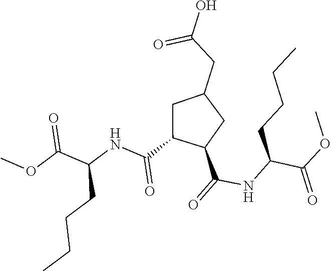 [SMILES: CCCC[C@H](NC(=O)[C@@H]1CC(CC(=O)O)C[C@H]1C(=O)N[C@@H](CCCC)C(=O)OC)C(=O)OC]

Tert-butyl [ethoxy(ethyl)phosphoryl]acetate (0.43 g, 1.7 mmol) was dissolved in dry THF (15 mL) and cooled to 0° C. under an atmosphere of nitrogen. NaHMDS (1.7 mL, 1.7 mmol) was added and the mixture was stirred for 30 minutes. The intermediate from step a of this example (0.36 g, 0.85 mmol, in 10 mL of THF) was added dropwise and the reaction was stirred for 30 minutes. The reaction was quenched with 1N aqueous HCL (100 mL) and extracted into ethyl acetate (3×, 50 mL). The combined organics were dried over sodium sulfate and concentrated. The residue was purified by ISCO Combi Flash reversed phase HPLC (25-95% acetonitrile in DI water with 0.1% TFA as modifier, 40 minute gradient. The pure fractions were pooled and lyophilized to afford 425 mg of di-ester cyclopentyl acetic acid as a clear oil. Positive ions found by LCMS [M+H]+=525.4. The alkene was reduced by stirring in methanol (25 mL) in the presence of 5% Pd/C (150 mg) under 1 atmosphere of hydrogen for 4 hours. The mixture was filtered through celite and concentrated. The residue was stirred in TFA (15 mL) at ambient temperature for 12 hours. The TFA was removed by rotary evaporator and the residue was purified by ISCO Combi Flash reversed phase HPLC (5-95% acetonitrile in DI water with 0.1% TFA as modifier, 40 minute gradient. The pure fractions were pooled and lyophilized to afford di methyl ester cyclopentyl acid as a clear oil. Yield of 334 mg, 83%, 3 steps. Positive ions found by LCMS [M+H]+=471.2

### Step c

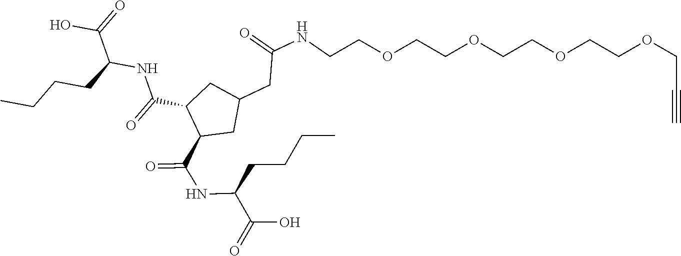 [SMILES: C#CCOCCOCCOCCOCCNC(=O)CC1C[C@@H](C(=O)N[C@@H](CCCC)C(=O)O)[C@H](C(=O)N[C@@H](CCCC)C(=O)O)C1]

A flask was charged with the intermediate from the previous step (0.303 g, 0.644 mmol), propagyl-PEG4-amine (0.179 g, 0.773 mmol) and DIEA (0.34 mL, 1.93 mmol) in DMF (2 mL) at room temperature. To this solution was added a prepared solution of HATU (0.294 g, 0.773 mmol) in DMF (1.22 mL) via a syringe pump at a rate of 2.5 mL/hr. The reaction mixture was continued stirring overnight then purified by RPLC without aqueous work up using Isco CombiFlash reversed phase HPLC (5-60% acetonitrile in DI water with 0.1% TFA as modifier). The pure fractions were combined and lyophilized to give propargyl-peg4-di-ester intermediate (0.41 g, 93%). Positive ions found by LCMS: [M+H]+=684.4

To a solution of dimethyl ester (0.41 g, 0.60 mmol) from a previous step in methanol/tetrahydrofuran/DI water (1:1:2, 15 mL) was added lithium hydroxide (0.043 g, 1.80 mmol). The reaction mixture was stirred at ambient temperature for 1 hour then acidified with glacial acetic acid to pH4. Methanol and tetrahydrofuran were stripped under reduced pressure. The mixture was purified by RPLC using Isco CombiFlash reversed phase HPLC (5-40% acetonitrile and DI water, using 0.1% TFA as modifier) to yield the title di-acid compound after lyophilizing, 0.411 g, 94%. Positive ions found by LCMS: [M+H]+=656.4.

### Step d

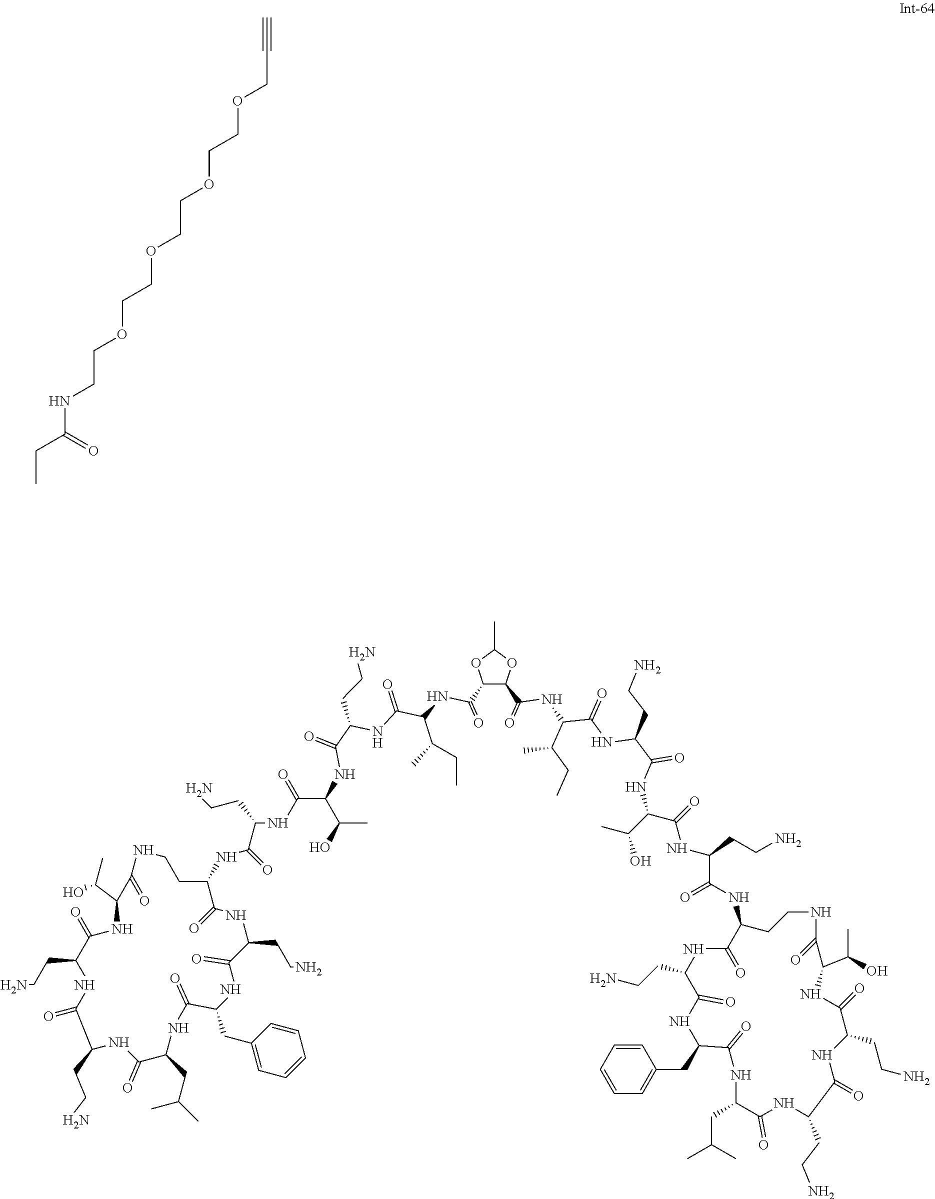

The title compound Int-64 was prepared from the previous di-acid intermediate and Int-7a with Thr2 extended (described in Example 67, step h), analogously to Example 90 step C. Yield 125 mg, 49%, 2 steps. Positive ions found by LCMS (M+3H⁺)/3=934.6, LCMS (M+4H⁺)/4=701.3, LCMS (M+5H⁺)/5=581.5.

### Example 100—Synthesis of Int-65

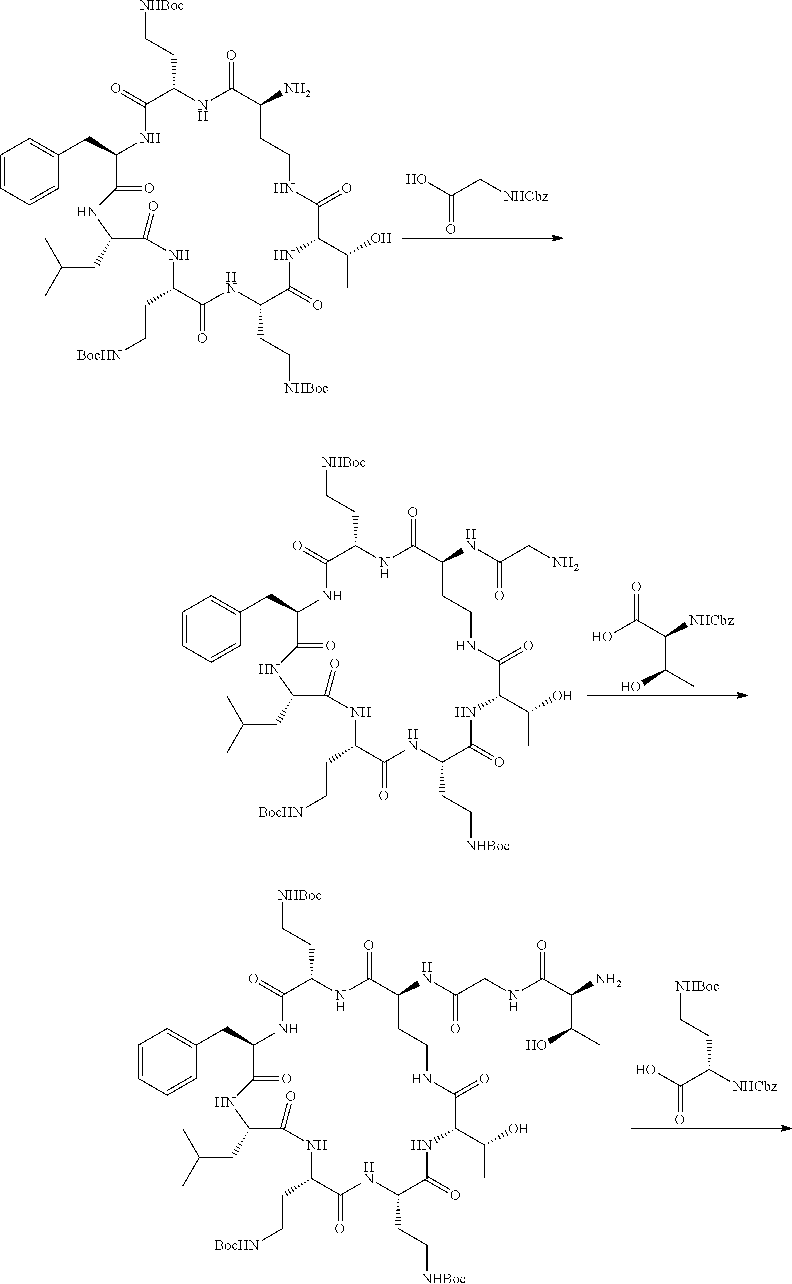

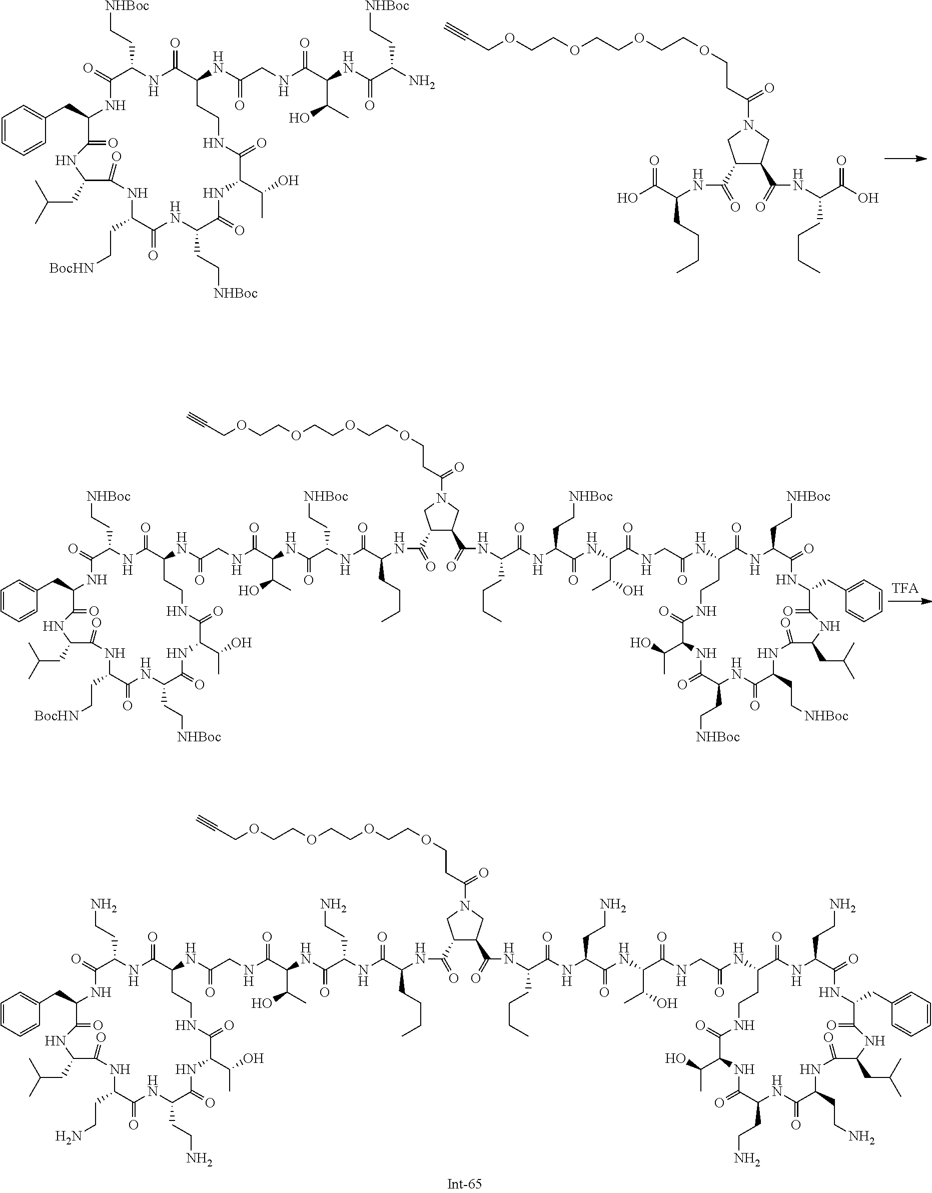

### Step a

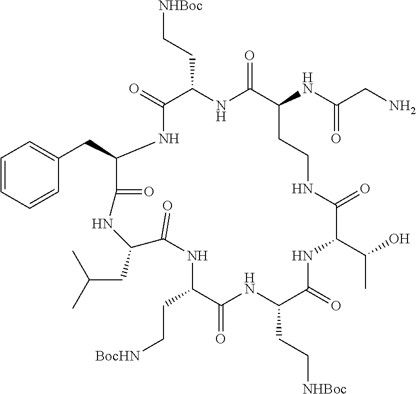 [SMILES: CC(C)C[C@@H]1NC(=O)[C@@H](Cc2ccccc2)NC(=O)[C@H](CCNC(=O)OC(C)(C)C)NC(=O)[C@@H](NC(=O)CN)CCNC(=O)[C@H]([C@@H](C)O)NC(=O)[C@H](CCNC(=O)OC(C)(C)C)NC(=O)[C@H](CCNC(=O)OC(C)(C)C)NC1=O]

To a mixture of PMB heptapeptide (4.24 g, 4 mmol, Example 2) and Z-Gly-OH (1 g, 4.8 mmol) in anhydrous DMF (8 mL) was added HATU (1.87 g, 4.9 mmol) in portions over 20 minutes, followed by DIPEA (936 mg, 7.2 mmol). After the reaction mixture was stirred for 15 minutes, it was poured into water (100 mL). The white solid product was collected by filtration and washed with water. The material was re-dissolved in MeOH (50 mL) and treated with Pd/C (5%) (1 g), then stirred under hydrogen overnight. Pd/C (5%) was then filtered, concentrated and purified by RPLC (150 g, 15 to 75% MeOH and water). Yield 4.02 g, 90%. Ions found by LCMS: [(M−Boc+2H)/2]⁺=510.4, [(M−3Boc+2H)/2]⁺=410.2

### Step b

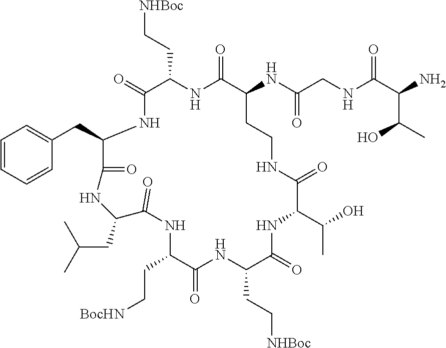 [SMILES: CC(C)C[C@@H]1NC(=O)[C@@H](Cc2ccccc2)NC(=O)[C@H](CCNC(=O)OC(C)(C)C)NC(=O)[C@@H](NC(=O)CNC(=O)[C@@H](N)[C@@H](C)O)CCNC(=O)[C@H]([C@@H](C)O)NC(=O)[C@H](CCNC(=O)OC(C)(C)C)NC(=O)[C@H](CCNC(=O)OC(C)(C)C)NC1=O]

To a mixture of the step-a product (4.02 g, 3.592 mmol) and Z-Thr-OH (980.3 mg, 3.87 mmol) in anhydrous DMF (5 mL) was added HATU (1.47 g, 3.87 mmol) in portions over 10 minutes, followed by DIPEA (755 mg, 5.8 mmol). After the addition, the reaction was stirred for 20 minutes and then poured into water (100 mL). The white solid product was collected by filtration and washed with water. The material was re-dissolved in MeOH (50 mL) and treated with Pd/C (5%) (1 g), and stirred under hydrogen overnight. Pd/C was then filtered, and the filtrate was concentrated and purified by RPLC (150 g column, 15 to 80% MeOH and water). Yield 3.68 g, 84%. Ion found by LCMS: [(M−2Boc+2H)/2]⁺=511.0

### Step c

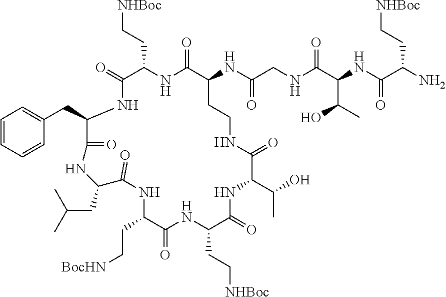 [SMILES: CC(C)C[C@@H]1NC(=O)[C@@H](Cc2ccccc2)NC(=O)[C@H](CCNC(=O)OC(C)(C)C)NC(=O)[C@@H](NC(=O)CNC(=O)[C@@H](NC(=O)[C@@H](N)CCNC(=O)OC(C)(C)C)[C@@H](C)O)CCNC(=O)[C@H]([C@@H](C)O)NC(=O)[C@H](CCNC(=O)OC(C)(C)C)NC(=O)[C@H](CCNC(=O)OC(C)(C)C)NC1=O]

A mixture of the step-b product (1.2 g, 0.984 mmol) and Z-Dab(Boc)-OH DCHA salt (605 mg, 1.13 mmol) was dissolved in anhydrous NMP (3 mL). It was treated with HATU (430 mg, 1.13 mmol) in portions over 5 minutes, followed by DIPEA (150 mg, 1.13 mmol). The reaction was stirred for 30 minutes and then directly purified by RPLC (100 g column, 40 to 100% MeOH and water). The collected fractions were concentrated by rotary evaporation to a white solid (Ion found by LCMS: [M−2Boc+2H)/2]⁺=678). The material was re-dissolved in MeOH (30 mL) and treated with Pd/C (5%), then stirred under hydrogen overnight. Pd/C was filtered, and the filtrate was concentrated by rotary evaporation and further dried under high vacuum. Yield 1.07 g, 76.6%. Ion found by LCMS: [(M−2Boc+2H)/2]⁺=611.

### Step d

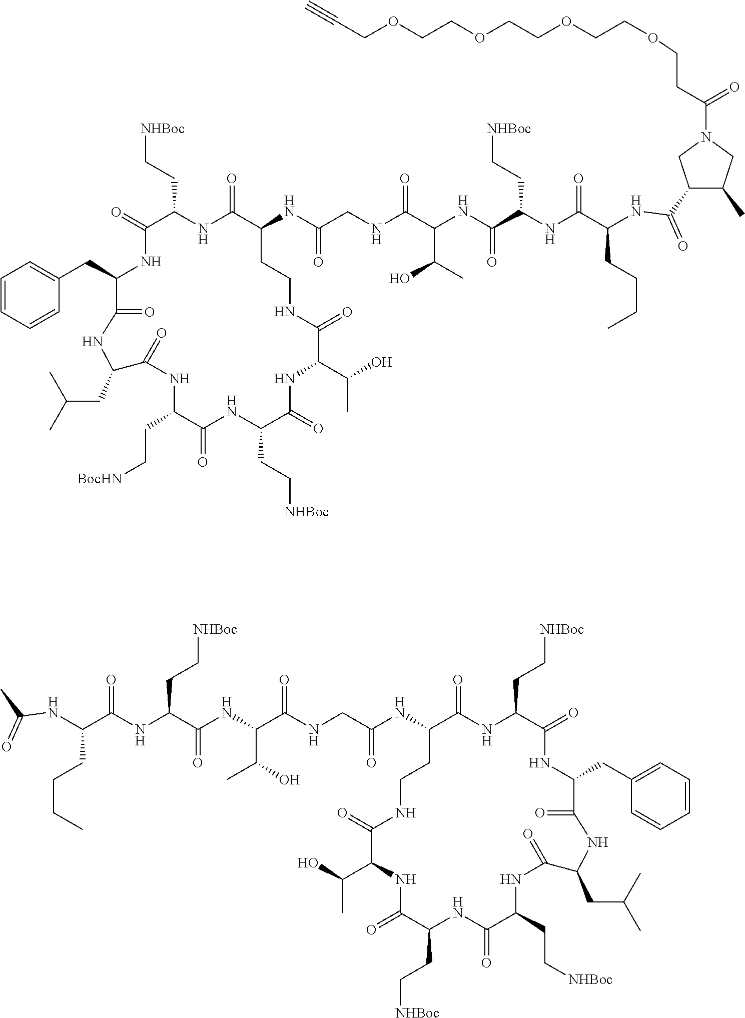

A mixture of the step-c product (313.6 mg, 0.2207 mmol) and diacid central linker (65.9 mg, 0.105 mmol, Example 41, INT-19) was dissolved in anhydrous DMF (1 mL). DIPEA (65 mg, 0.5 mmol) and HATU (83.9 mg, 0.2207 mmol) were added in portions over 5 minutes. The reaction was stirred for 30 minutes and then directly purified by RPLC (50 g, 40 to 100% MeOH and water). Yield 165 mg, 50%. Ions found by LCMS: [M−3Boc+3H)/3]⁺=1044.6, [M−4Boc+3H)/3]⁺=1011.3, [M−6Boc+3H)/3]⁺=944.4.

### Step e

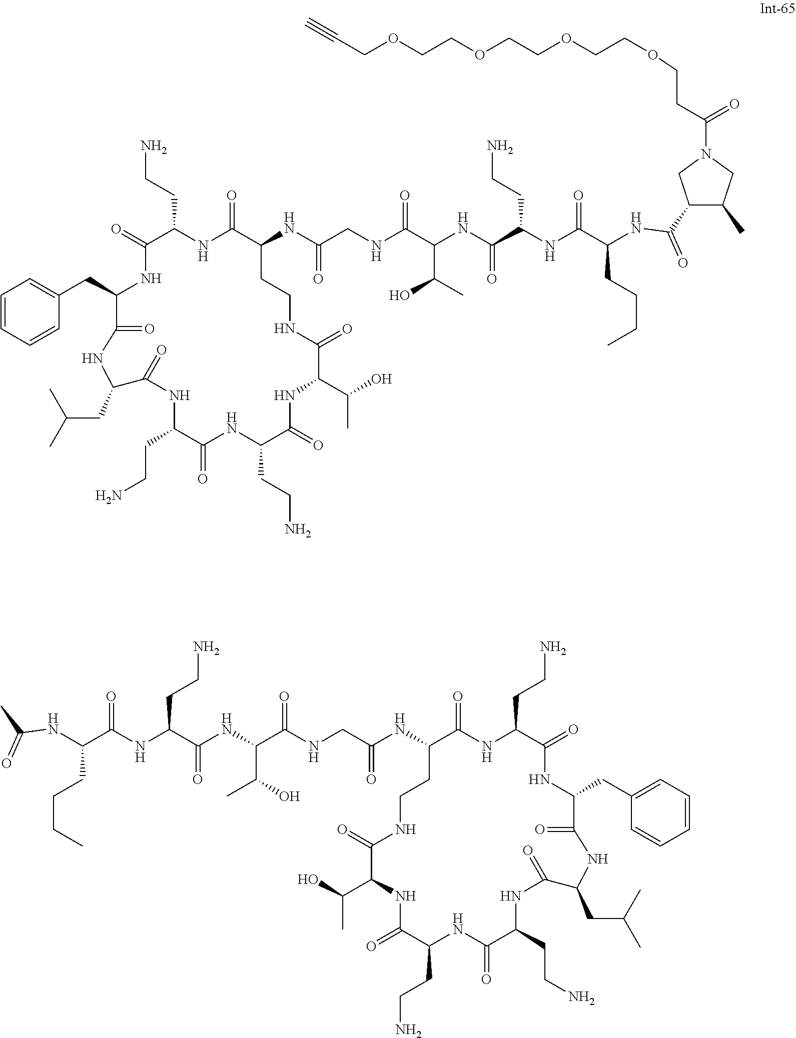

The step-d product (165 mg, 0.0521 mmol) was dissolved in TFA/DCM (1:1, 1 mL), and the solution was stirred for 30 minutes. It was concentrated and directly purified by RPLC (50 g column, 5 to 30% acetonitrile and water, using 0.1% TFA as modifier). Yield of Int-65 was 160 mg, 87%. Ions found by LCMS: [M+4H)/4]⁺=658.4, [M+5H)/5]⁺=527.1. [M+6H)/6]⁺=439.4, [M+7H)/7]⁺=376.8.

### Example 101—Synthesis of Int-66

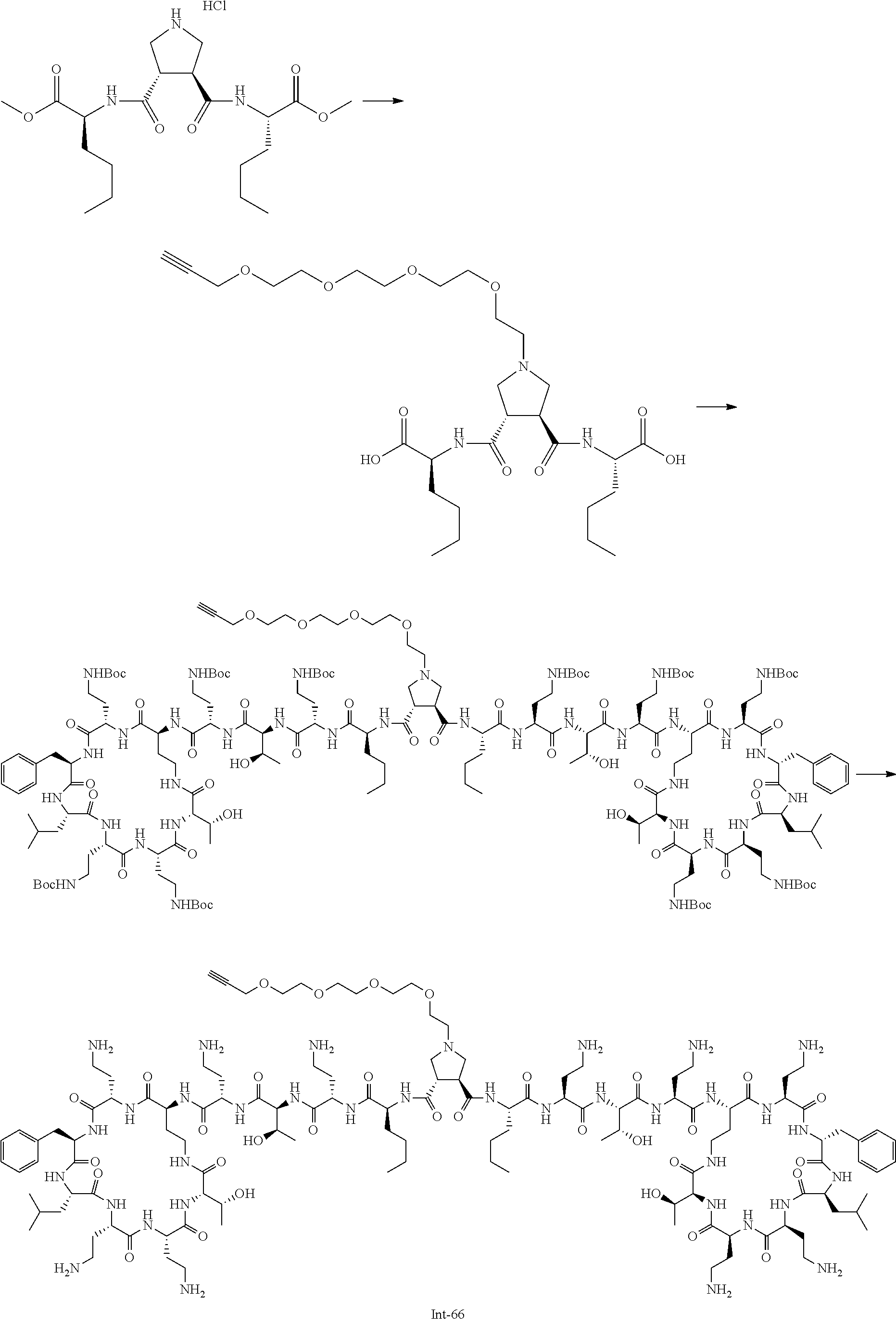

### Step a

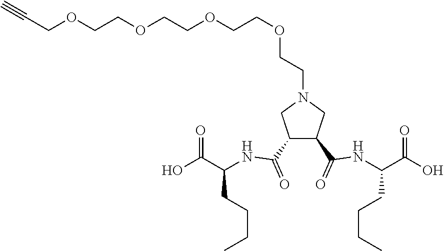 [SMILES: C#CCOCCOCCOCCOCCN1C[C@@H](C(=O)N[C@@H](CCCC)C(=O)O)[C@H](C(=O)N[C@@H](CCCC)C(=O)O)C1]

To a solution of intermediate from Example 7, INT-8a (228.1 mg, 0.507 mmol) in anhydrous ethanol (2 mL) was added DIPEA (198 mg, 1.52 mmol) and propargyl-PEG4-bromide (180 mg, 0.608 mmol). After the mixture was heated at 80° C. for 30 hours, it was cooled to room temperature and then placed in an ice-water bath. THF (1 mL) and a solution of LiOH (105 mg, 2.5 mmol) in water (2 mL) were added. The reaction was stirred for 3 hours. It was acidified by 4N HCl solution in dioxane (0.7 mL) and directly purified by RPLC (50 g, 5 to 70% acetonitrile and water). Yield 92.9 mg, 30.6%. Ion found by LCMS: [M+H]⁺=600.

### Step b

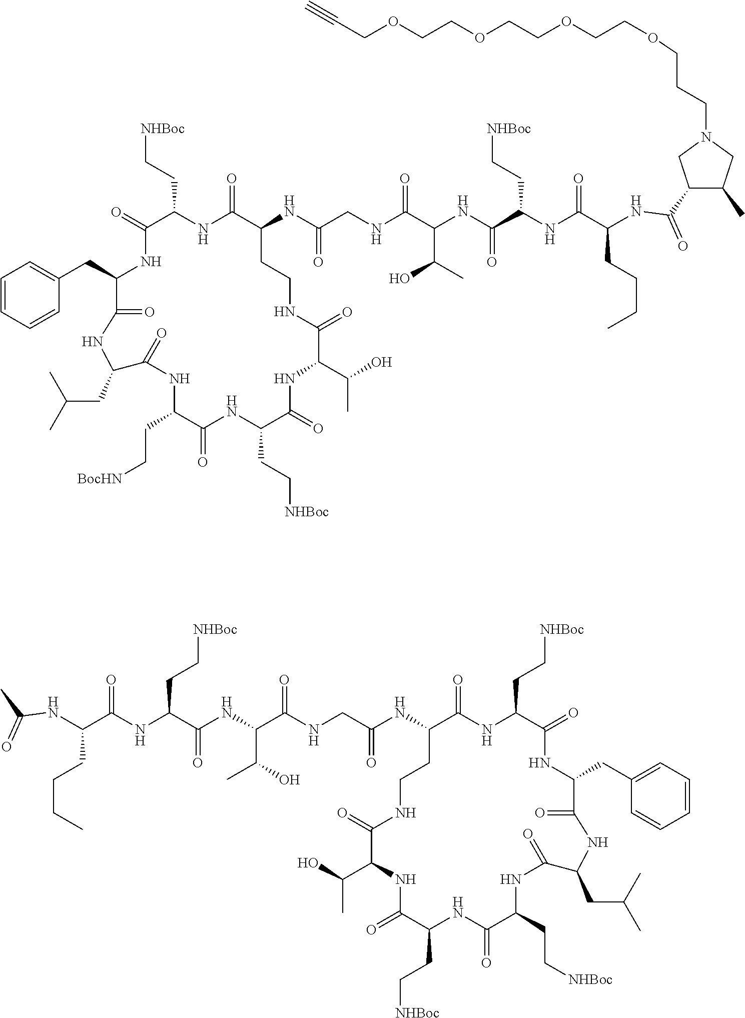

To a solution of the step-a product (32 mg, 0.05336 mmol) and Int-7a (Example 8, step a) (183.6 mg, 0.1174 mmol) in anhydrous DMF (1 mL), was added DIPEA (42 mg, 0.4 mmol) and HATU (40.6 mg, 0.107 mmol). The resulting solution was stirred for 30 minutes and was purified by RPLC (50 g column, 20 to 100% MeOH and water). Yield 189 mg, 96%. Ion found by LCMS: [(M−2Boc+3H)/3]⁺=1164.4, [(M−3Boc+3H)/3]⁺=1131, [(M−4Boc+3H)/3]⁺=1097.

### Step c

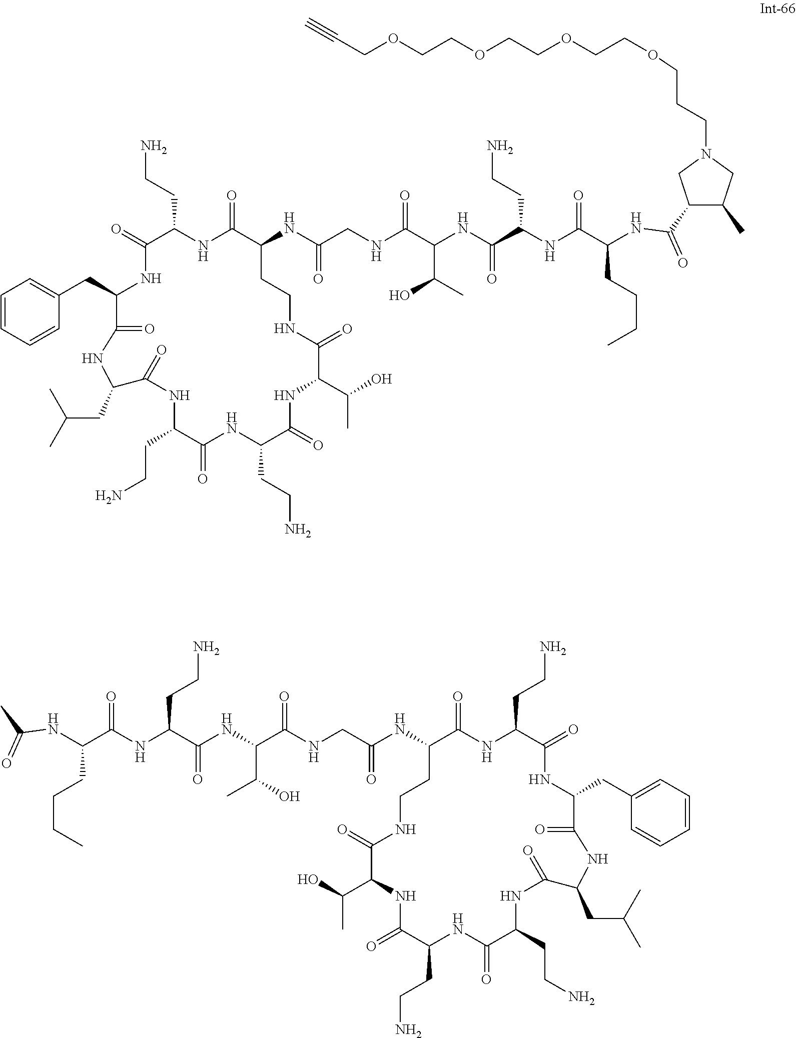

The step-b product (189 mg, 0.0512 mmol) was dissolved in TFA (0.5 mL) and stirred for 15 minutes. It was directly purified by RPLC (50 g column, 0 to 50% acetonitrile and water, using 0.1% TFA as modifier). Yield of Int-66 was 103 mg, 51%. Ions found by LCMS: [(M+5H)/5]⁺=538.6, [(M+6H)/6]⁺=449.2, [(M+7H)/7]⁺=385.0, [(M+8H)/8]⁺=337.0.

### Example 102—Synthesis of Int-67

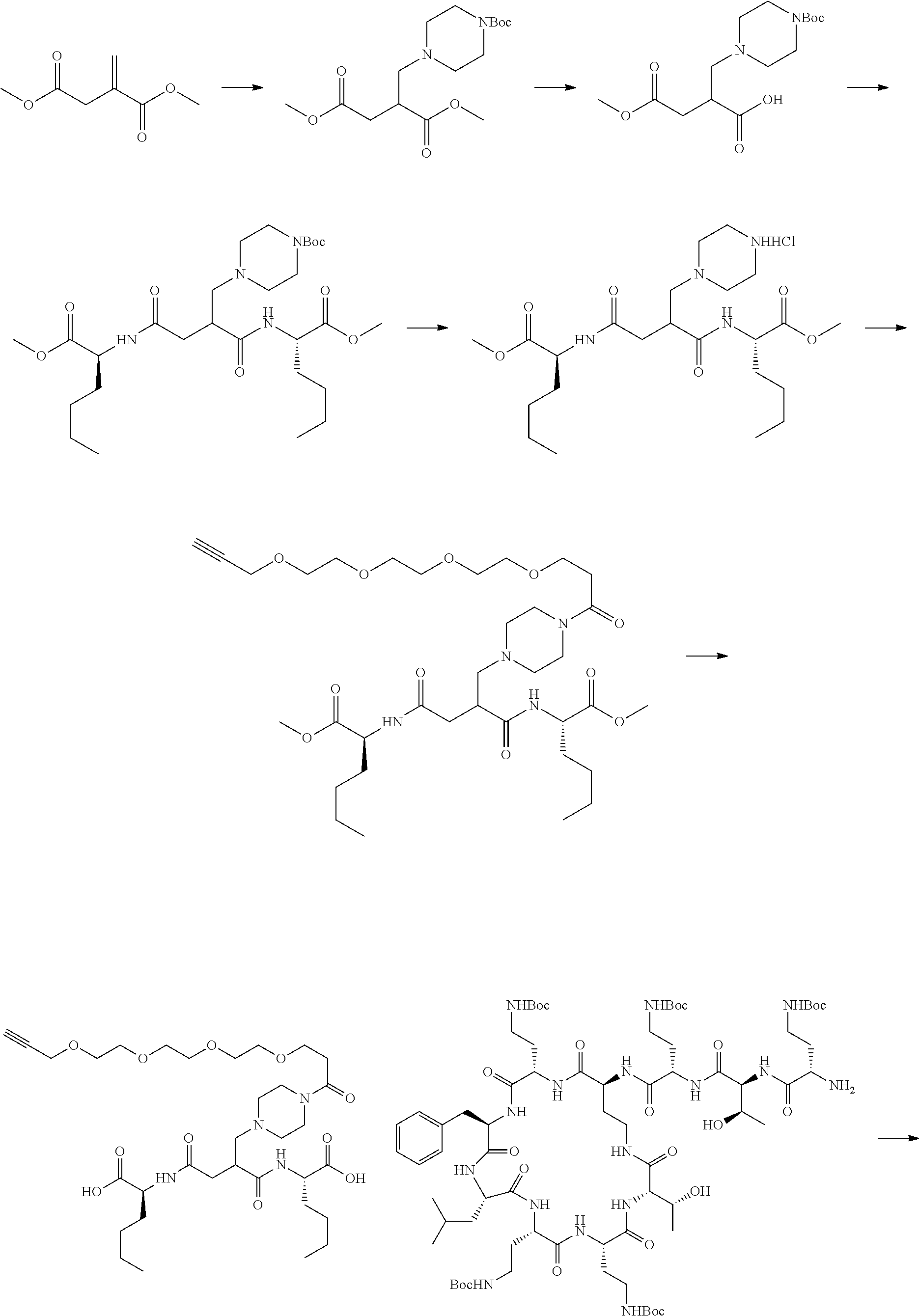

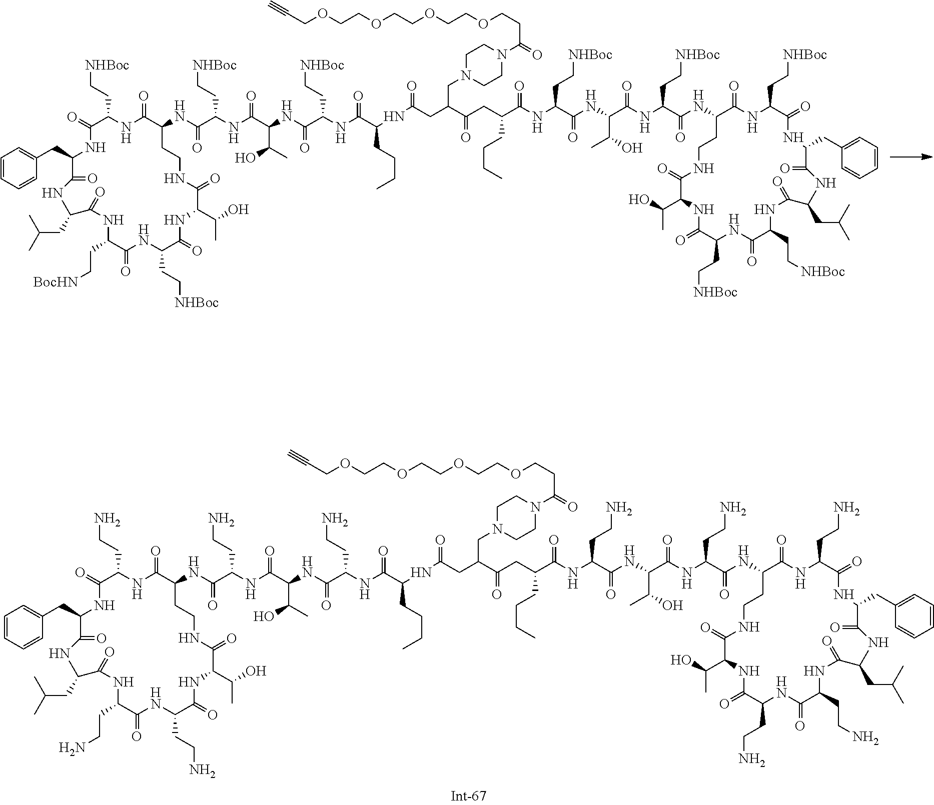

### Step a

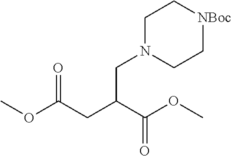 [SMILES: COC(=O)CC(CN1CCN(C(=O)OC(C)(C)C)CC1)C(=O)OC]

To a solution of dimethyl itaconate (1.58 g, 10 mmol) in MeOH (8 mL) was treated with t-butyl piperazine-1-carboxylate (1.88 g, 10 mmol). The mixture was heated at 60° C. for 1 day. It was then purified by HPLC (0 to 20% acetonitrile and water, using 0.1% TFA as modifier). Yield 1.175 g, 34.1° %. Ion found by LCMS: [M+H]⁺=345.0.

### Step b

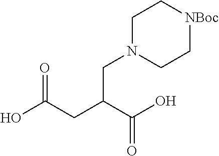 [SMILES: CC(C)(C)OC(=O)N1CCN(CC(CC(=O)O)C(=O)O)CC1]

The step-b product (1.175 g, 3.41 mmol) was dissolved in MeOH (5 mL) and cooled in an ice-water bath. It was treated with a solution of LiOH monohydrate (378 mg, 9 mmol) in water (9 mL). After the reaction was stirred at 0° C. to room temperature overnight, it was re-cooled in an ice-water bath and acidified by 4N HCl solution in dioxane (2 mL). The mixture was partially concentrated by rotary evaporation at room temperature. The residue was purified by RPLC (150 g column, 0 to 80% MeOH and water). Yield 336.5 mg, 31.2%. Ion found by LCMS: [M+H]⁺=317.2.

### Step c

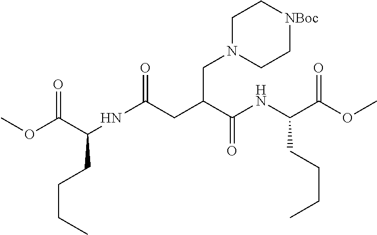

A mixture of the step b product (336.5 mg, 1.064 mmol) and H-NIe-OMe HCl (464 mg, 2.552 mmol) was dissolved in anhydrous NMP (3 mL) and DIPEA (520 mg, 4 mmol). To this was added HATU (810 mg, 2.13 mmol) in portions over 10 minutes. The reaction was stirred for another 30 minutes, then purified by RPLC (100 g column, 10 to 75% acetonitrile and water, using 0.1% TFA as modifier). Yield 261.7 mg, 43.2%. Ion found by LCMS: [M+H]⁺=571.4.

### Step d

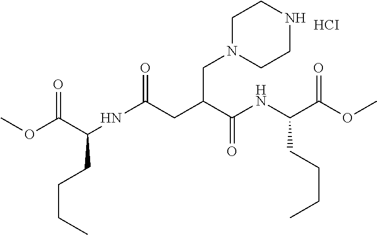

The step c product (261.7 g, 0.459 mmol) was dissolved in THF (5 mL), and 4N HCl in dioxane (2.5 mL). The mixture was stirred at room temperature for 4 hours and then directly purified by RPLC (50 g, 0 to 80% acetonitrile and water). Yield 144.3 mg, 66.9%. Ion found by LCMS: [M+H]⁺=471.4.

### Step e

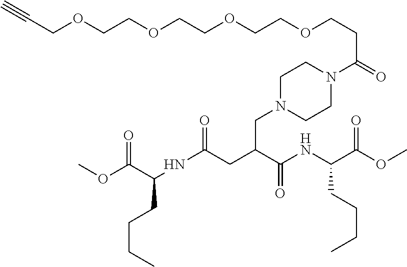 [SMILES: C#CCOCCOCCOCCOCCC(=O)N1CCN(CC(CC(=O)N[C@@H](CCCC)C(=O)OC)C(=O)N[C@@H](CCCC)C(=O)OC)CC1]

A mixture of the step d product (144.3 mg, 0.307 mmol) and propargyl-PEG4-acid (104 mg, 0.4 mmol) was dissolved in anhydrous DMF (1 mL) and DIPEA (130 mg, 1 mmol). HATU (152 mg, 0.4 mmol) was added, and the reaction was stirred for 30 minutes. It was then purified by RPLC (100 g, 5 to 100% acetonitrile and water). Yield 165 mg, 75.6%. Ion found by LCMS: [M+H]⁺=713.4.

### Step f

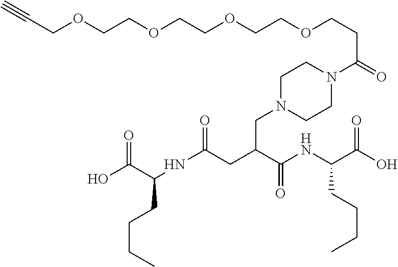 [SMILES: C#CCOCCOCCOCCOCCC(=O)N1CCN(CC(CC(=O)N[C@@H](CCCC)C(=O)O)C(=O)N[C@@H](CCCC)C(=O)O)CC1]

The step e product (165 mg, 0.232 mmol) was dissolved in MeOH (2 mL) and cooled in an ice-water bath. It was treated with a solution of LiOH monohydrate (21 mg, 0.5 mmol) in water (1 mL). After the reaction was stirred for 4 hours, it was acidified by 4N HCl solution in dioxane (0.2 mL) and directly purified by HPLC (5 to 35% acetonitrile and water). Yield 46.4 mg, 29.2%. Ion found by LCMS: [M+H]⁺=685.

### Step g

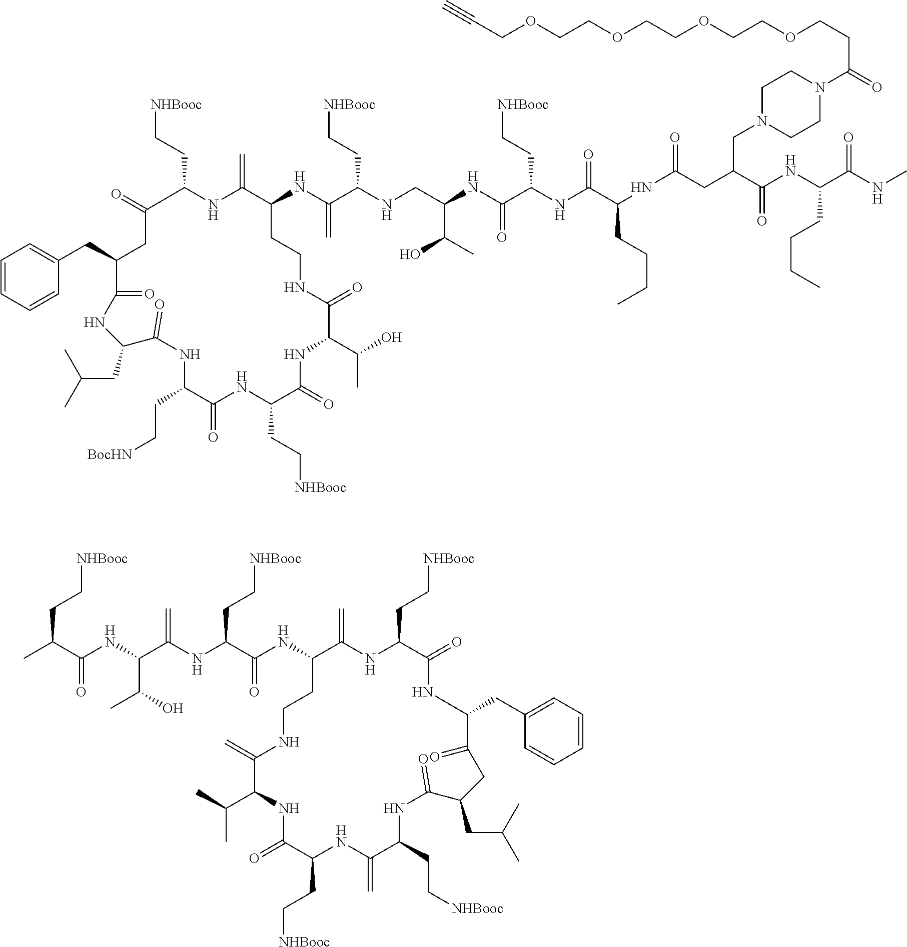

The title compound was prepared analogously to Example 101, step-b, using the step-f diacid and Int-7a (Example 8, step a). Yield 246 mg, 96.1%. Ions found by LCMS: [(M−2Boc+3H)/3]⁺=1192.4, [(M−3Boc+3H)/3]⁺=1159.1, [(M−4Boc+3H)/3]⁺=1125.8.

### Step h

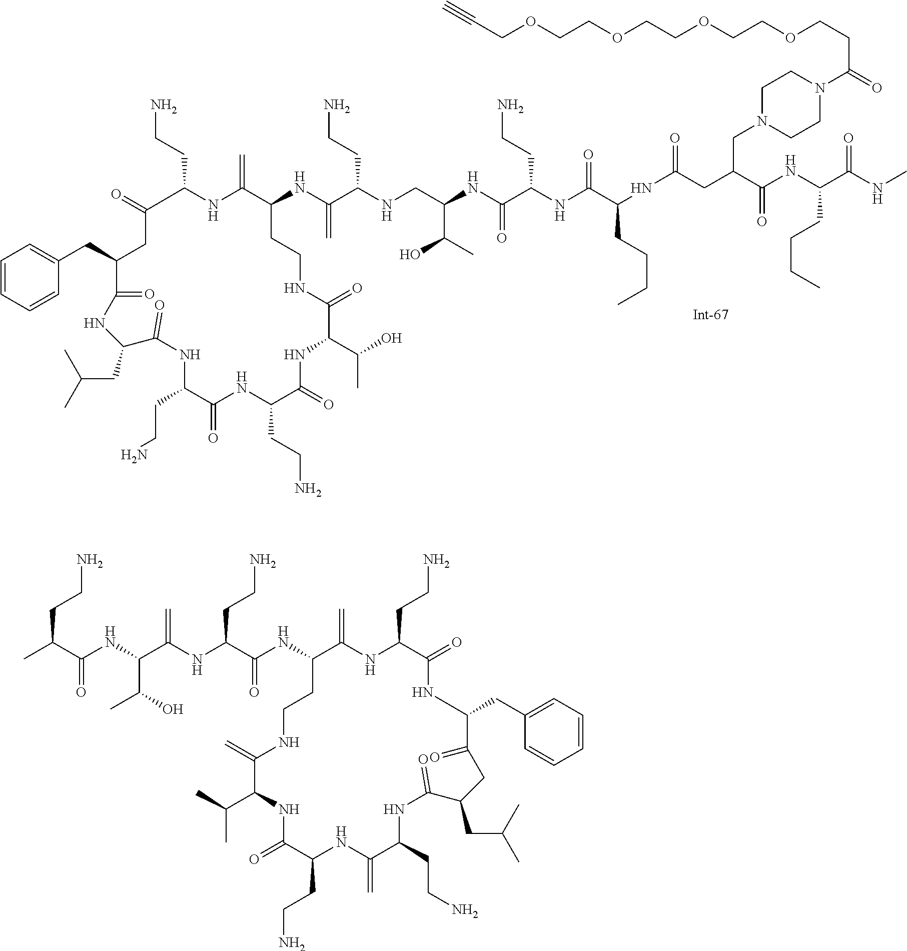

The title compound Int-87 was prepared analogously to Example 101, step-c, using the product from step-g of this example. Yield 115 mg, 43.6%. Ions found by LCMS: [(M+4H)/4]⁺=694.4, [(M+4H)/5]⁺=555.8, [(M+6H)/6]⁺=463.2, [(M+7H)/7]⁺=397.2.

### Example 103—Synthesis of Int-68

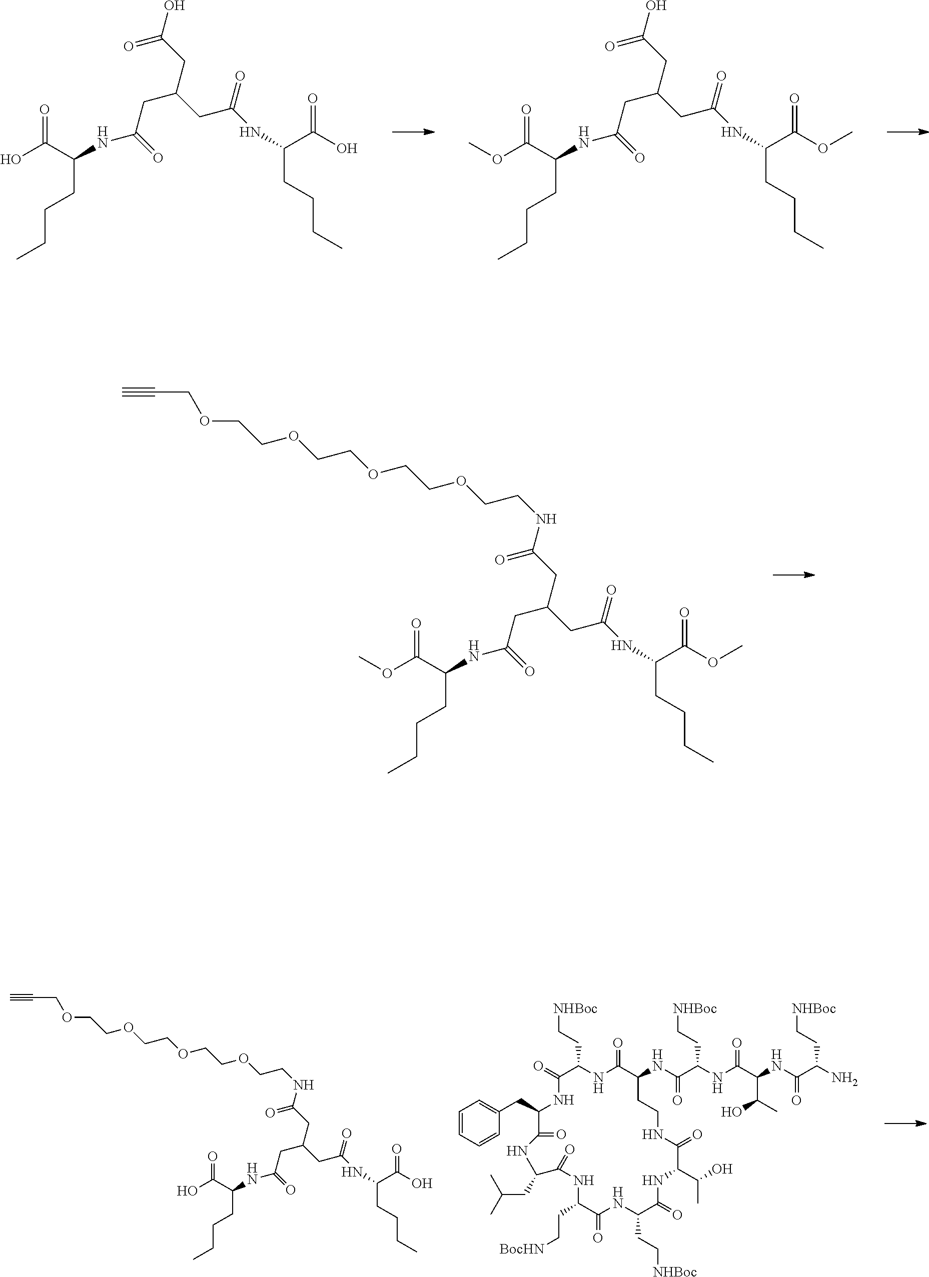

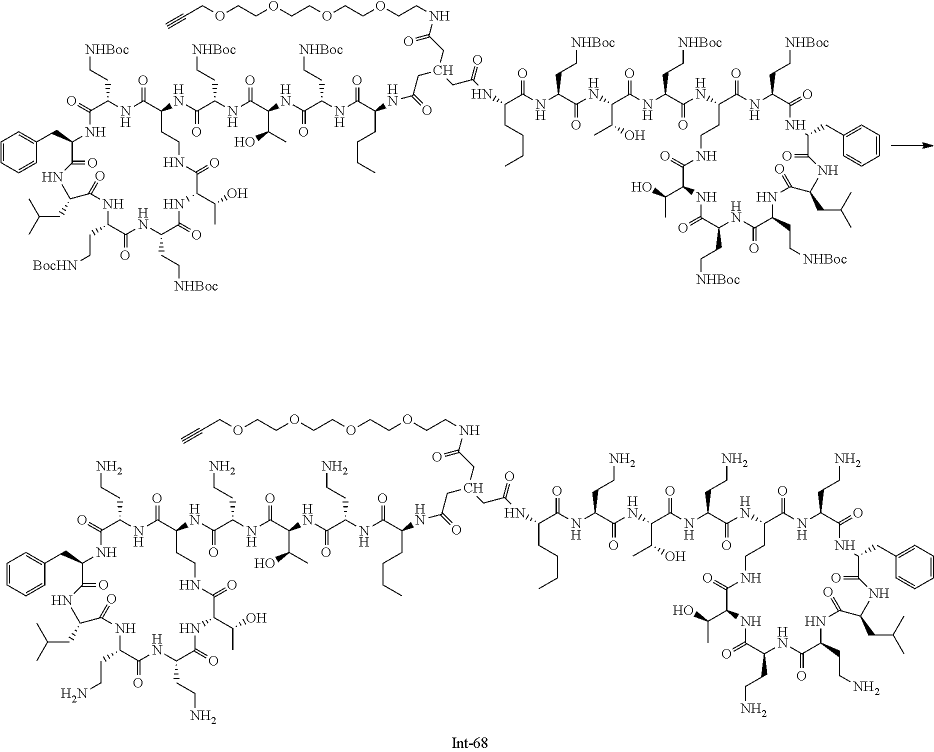

### Step a

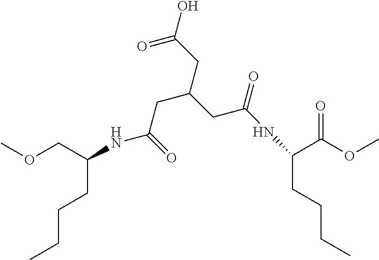

A mixture of 3-(carboxymethyl)pentanedioic acid (190.2 mg, 1.0 mmol) and H-NIe-OMe HCl (400 mg, 2.2 mmol) was dissolved in anhydrous NMP (2 mL) and DIPEA (546 mg, 4.2 mmol). HATU (760.4 mg, 2.0 mmol) was added in portions over 10 minutes, and the reaction was stirred for 1 hour. It was then purified by RPLC (100 g column, 5 to 80% acetonitrile and water). Yield 162.5 mg, 36.6%. Ion found by LCMS: [M+H]⁺=445.2.

### Step b

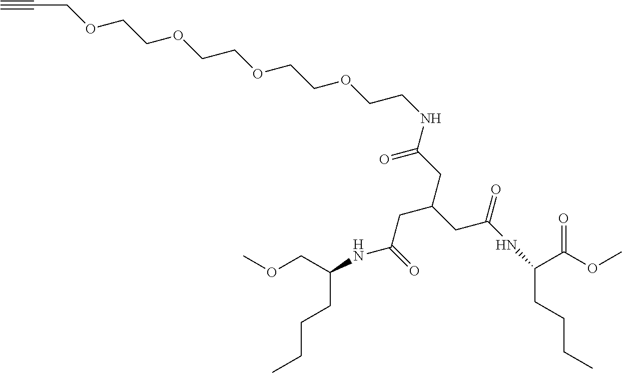 [SMILES: C#CCOCCOCCOCCOCCNC(=O)CC(CC(=O)N[C@@H](CCCC)COC)CC(=O)N[C@@H](CCCC)C(=O)OC]

A mixture of the step a product (162.5 mg, 0.366 mmol) and propargyl-PEG4-amine (101.8 mg, 0.392 mmol) was dissolved in anhydrous DMF (1 mL) and DIPEA (78 mg, 0.6 mmol). HATU (140 mg, 0.366 mmol) was added, and the reaction was stirred for 30 minutes. It was then purified by RPLC (50 g column, 10 to 80% acetonitrile and water). Yield 159.1 mg, 66.1%. Ion found by LCMS: [M+H]⁺=658.4.

### Step c

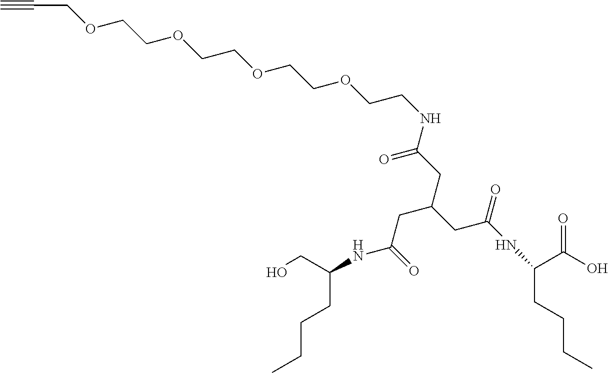 [SMILES: C#CCOCCOCCOCCOCCNC(=O)CC(CC(=O)N[C@H](CO)CCCC)CC(=O)N[C@@H](CCCC)C(=O)O]

The step-b product (159.1 mg, 0.242 mmol) was dissolved in MeOH (2 mL) and cooled in an ice-water bath. It was treated with a solution of LiOH monohydrate (25.4 mg, 0.6 mmol) in water (1 mL). After the reaction stirred for 4 hours, it was acidified with 4N HCl solution in dioxane (0.3 mL) and directly purified by RPLC (50 g, 5 to 60% acetonitrile and water). Yield 67.1 mg, 44%. Ion found by LCMS: [M+H]⁺=630.0.

### Step d

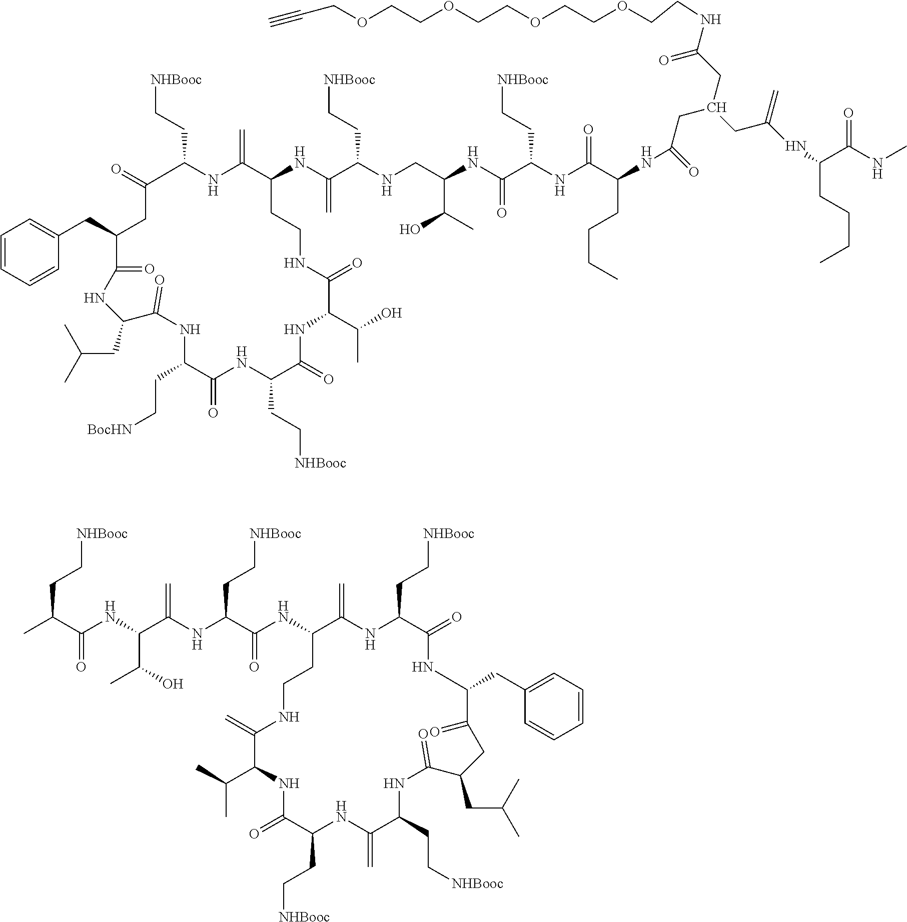

The title compound was prepared analogously to Example 101, step-b, using the diacid from step c and Int-7a (described in Example 8, step a). Yield 247.4 mg, 62.4%. Ions found by LCMS: [(M−2Boc+3H)/3]⁺=1174, [(M−3Boc+3H)/3]⁺=1140.7, [(M−4Boc+3H)/3]⁺=1107.4, [(M−5Boc+3H)/3]⁺=1074, [(M−6Boc+3H)/3]⁺=1040.7.

### Step e

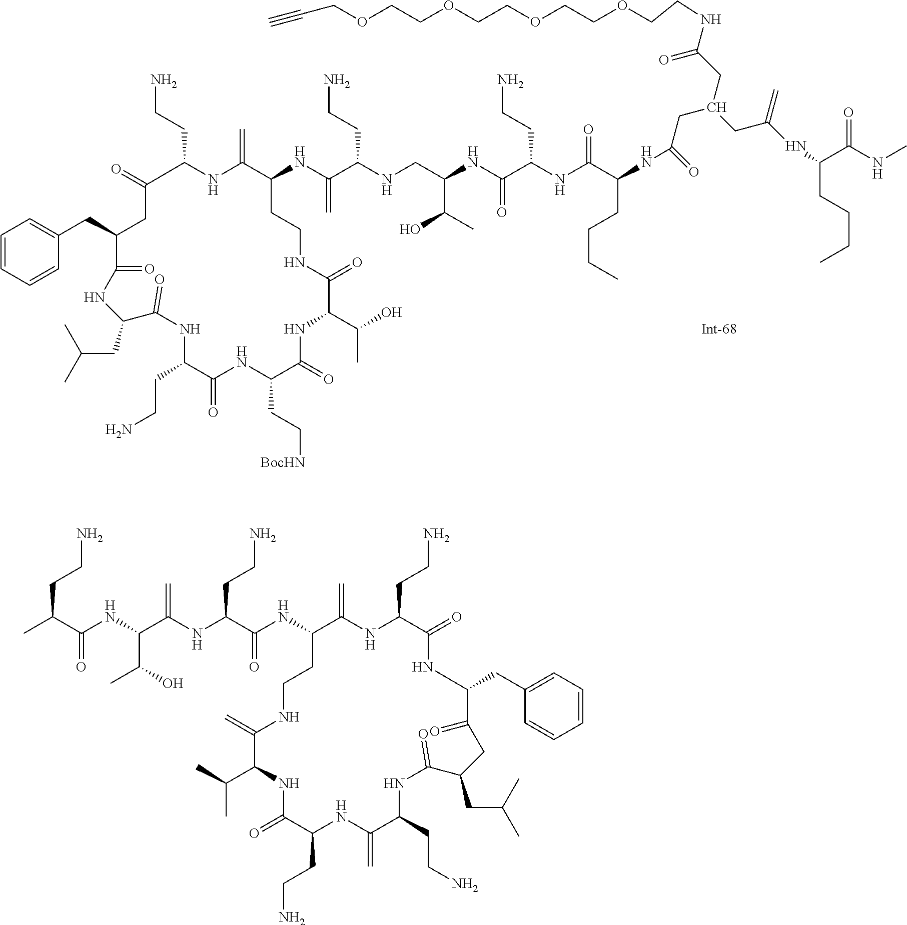

The title compound Int-68 was prepared analogously to Example 101, step-c, using the step-d product from this example. Yield 197.5 mg, 76.9%. Ions found by LCMS: [(M+4H)/4]⁺=680.6. [(M+4H)/5]⁺=544.6, [(M+6H)/6]⁺=454.2, [(M+7H)/7]⁺=389.4, [(M+8H)/8]⁺=340.8.

### Example 104—Synthesis of Int-69

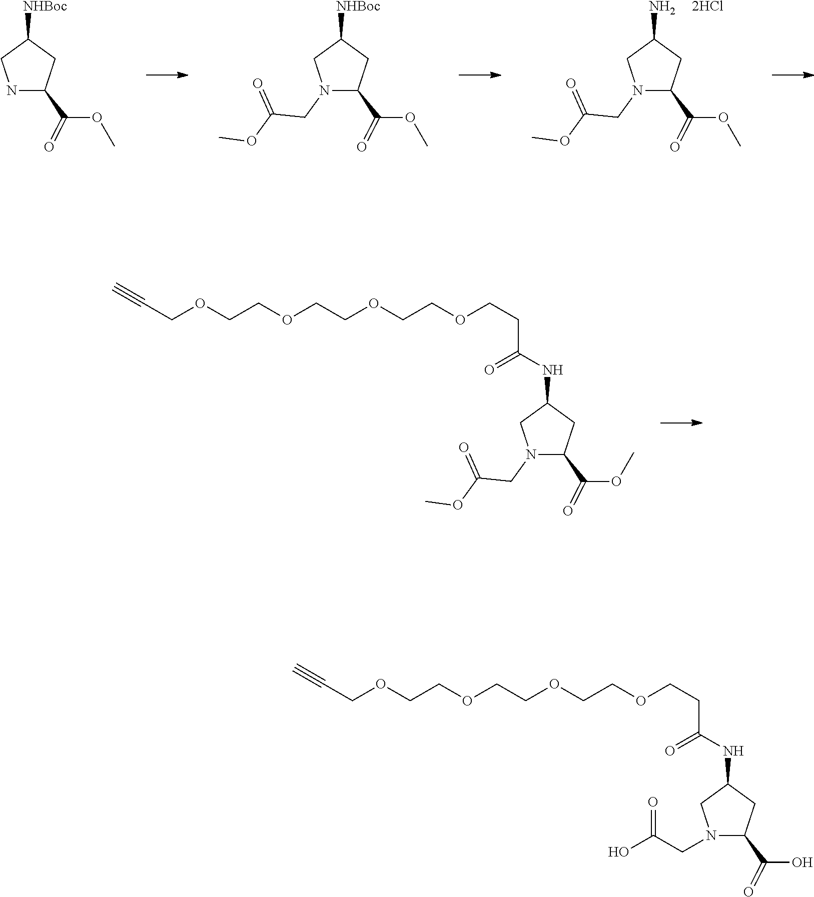

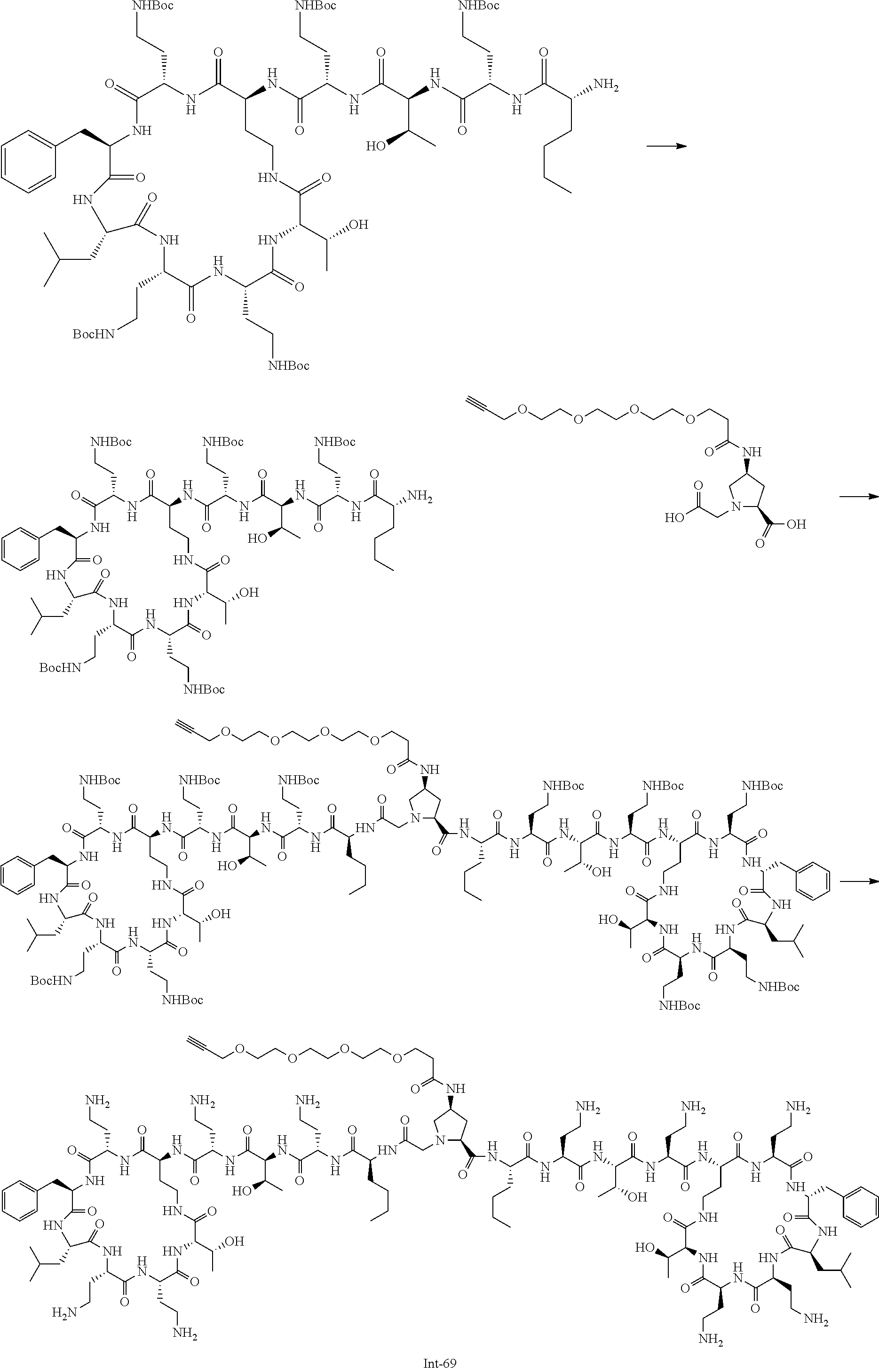

### Step a

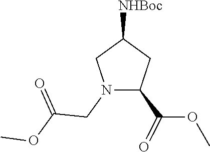

To a suspension of (4S)-Boc-amino-L-Proline methyl ester HCl salt (1.222 g, 5 mmol) in DCM (6 mL) and DMF (2 mL) was added benzyl 2-bromoacetate (1.375 g, 6 mmol) and DIPEA (975 mg, 7.5 mmol). The mixture was stirred at room temperature for 3 days. It was then concentrated and purified by RPLC (100 g column, 15 to 75% acetonitrile and water, using 0.1% TFA as the modifier). Yield 1.71 g, 87.2%. Ion found by LCMS: [M+H]⁺=393.2.

### Step b

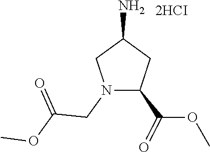

The step a product was dissolved in DCM (5 mL) and treated with 4N HCl solution in dioxane (5 mL). The mixture was heated at ˜40° C. for 30 minutes. It was then concentrated and re-dissolved in acetonitrile and water for lyophilization. Yield 1.6 g as di-HCl salt. Ion found by LCMS: [M+H]⁺=293.2.

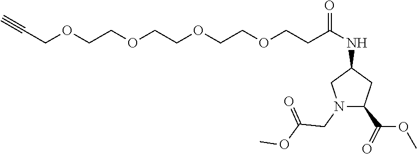 [SMILES: C#CCOCCOCCOCCOCCC(=O)N[C@H]1C[C@@H](C(=O)OC)N(CC(=O)OC)C1]

### Step c

To a solution of propargyl-PEG4 acid (520.6 mg, 2 mmol) in anhydrous DMF (3 mL) was added HATU (760.4 mg, 2 mmol). After the solid dissolved, the step b product (804 mg, 2.2 mmol) was added, followed by DIPEA (910 mg, 7 mmol). The resulting mixture was stirred for 1 hour and then directly purified by RPLC (100 g, 15 to 67% acetonitrile and water). Yield 629 mg, 58.9%. Ion found by LCMS: [M+H]⁺=535.2.

### Step d

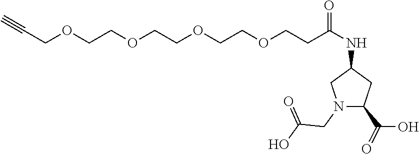 [SMILES: C#CCOCCOCCOCCOCCC(=O)N[C@H]1C[C@@H](C(=O)O)N(CC(=O)O)C1]

The step c product (629.1 mg, 1.178 mmol) was dissolved in MeOH (3 mL). This solution was cooled in an ice-water bath then treated with a solution of LIOH monohydrate (126 mg, 3 mmol) in water (3 mL). After the mixture was stirred for 3 hours, it was then acidified by 4N HCl solution in dioxane (1 mL). The organic solvents were removed by rotary evaporation, and the residue was purified by RPLC (100 g column, 0 to 50% acetonitrile and water). Yield 478 mg, 94.3%. Ion found by LCMS: [M+H]⁺=431.2

### Step e. Preparation of Int-75

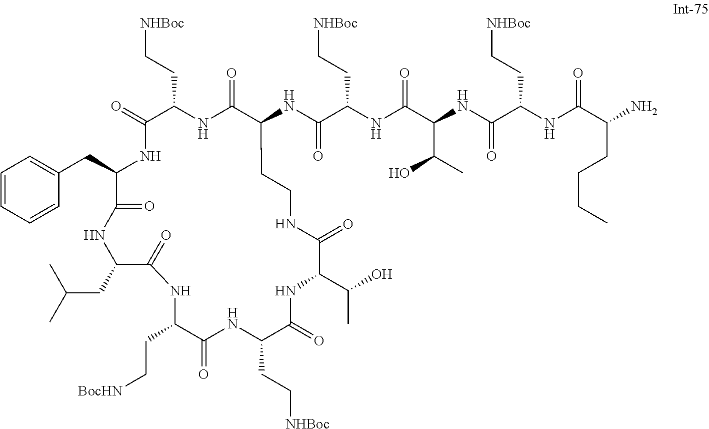 [SMILES: CCCC[C@@H](N)C(=O)N[C@@H](CCNC(=O)OC(C)(C)C)C(=O)N[C@H](C(=O)N[C@@H](CCNC(=O)OC(C)(C)C)C(=O)N[C@H]1CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](Cc2ccccc2)NC(=O)[C@H](CCNC(=O)OC(C)(C)C)NC1=O)[C@@H](C)O]

To a mixture of Int-7a (2 g, 1.28 mmol, Example 8, step a) and Z-L-norleucine (407.1 mg, 1.535 mmol) in anhydrous DMF (3 mL) was added HATU (583.6 g, 1.535 mmol) in portions over 10 minutes, followed by DIPEA (390 mg, 3 mmol). After the reaction mixture was stirred for 1 hour, it was poured into water (60 mL). The white solid product was collected by filtration and washed with water. The material was re-dissolved in MeOH (30 mL) and treated with Pd/C (5%) (1 g). The mixture was stirred under hydrogen overnight. Pd/C was then filtered, and the filtrate was concentrated and purified by RPLC (150 g, 40 to 95% MeOH and water). Yield of Int-75 was 2.02 g, 94.3%. Ion found by LCMS: [(M−Boc+2H)/2]+=789.2.

### Step f

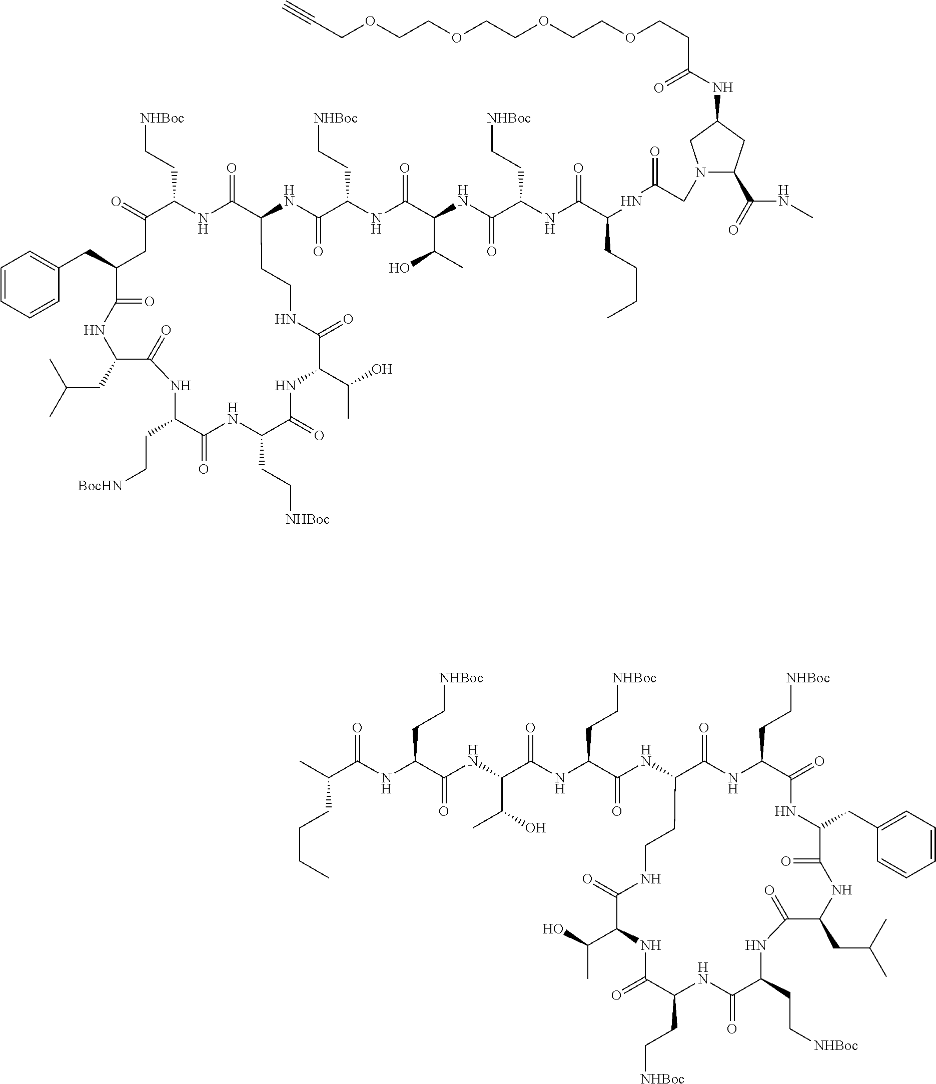

The title compound was prepared analogously to Example 101, step-b, using the step-e and step-d intermediates described in this example. Yield 244.4 mg, 65.3%. Ion found by LCMS: [(M−2Boc+3H)/3]⁺=1183.0, [(M−3Boc+3H)/3]⁺=1149.7, [(M−4Boc+3H)/3]⁺=1116.4, [(M−5Boc+3H)/3]⁺=1083.3, [(M−6Boc+3H)/3]⁺=1049.2, [(M−7Boc+3H)/3]⁺=1016.2.

### Step g

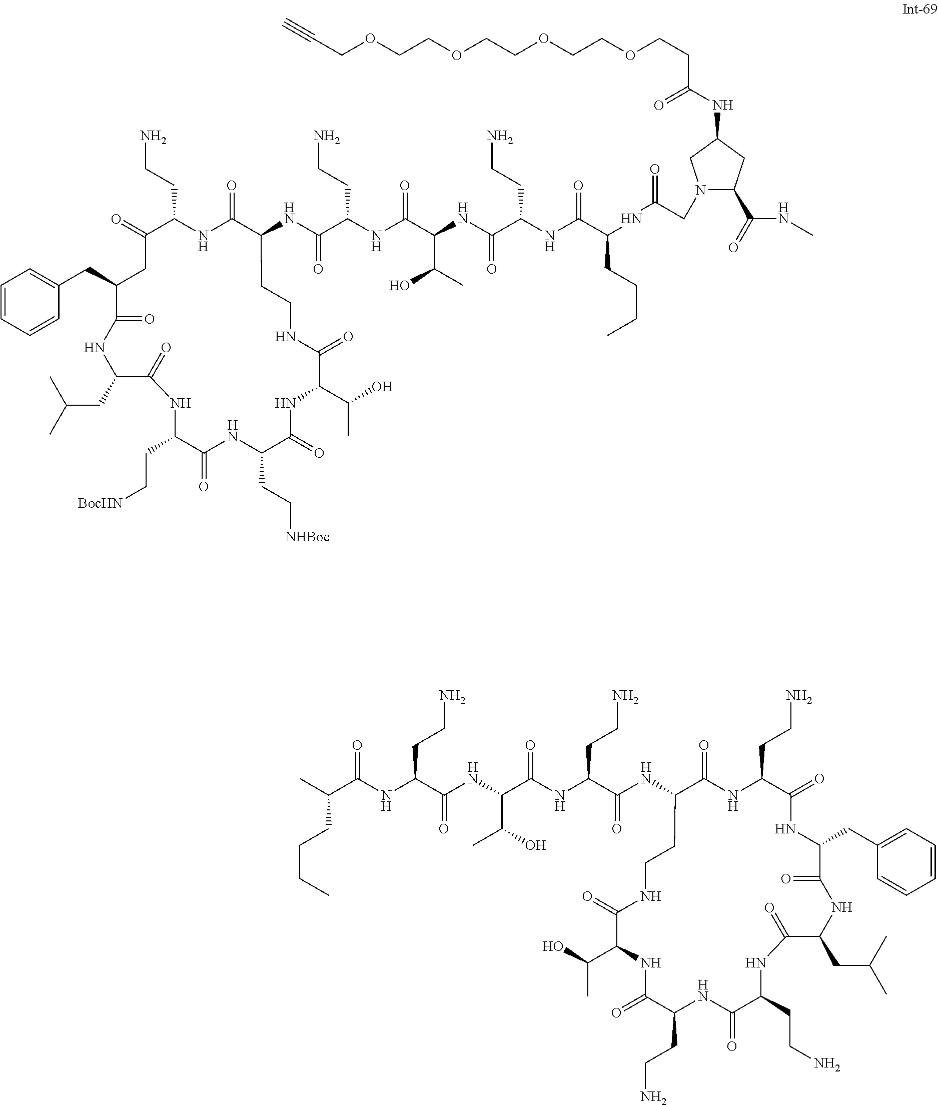

The title compound Int-69 was prepared analogously to Example 101, step-c, using the step-f product from this example. Yield 177.8 mg, 67.9%. Ions found by LCMS: [(M+4H)/4]⁺=687.5. [(M+5H)/5]⁺=550.1, [(M+6H)/6]⁺=458.6, [(M+7H)/7]⁺=393.2.

### Example 105—Synthesis of Int-70

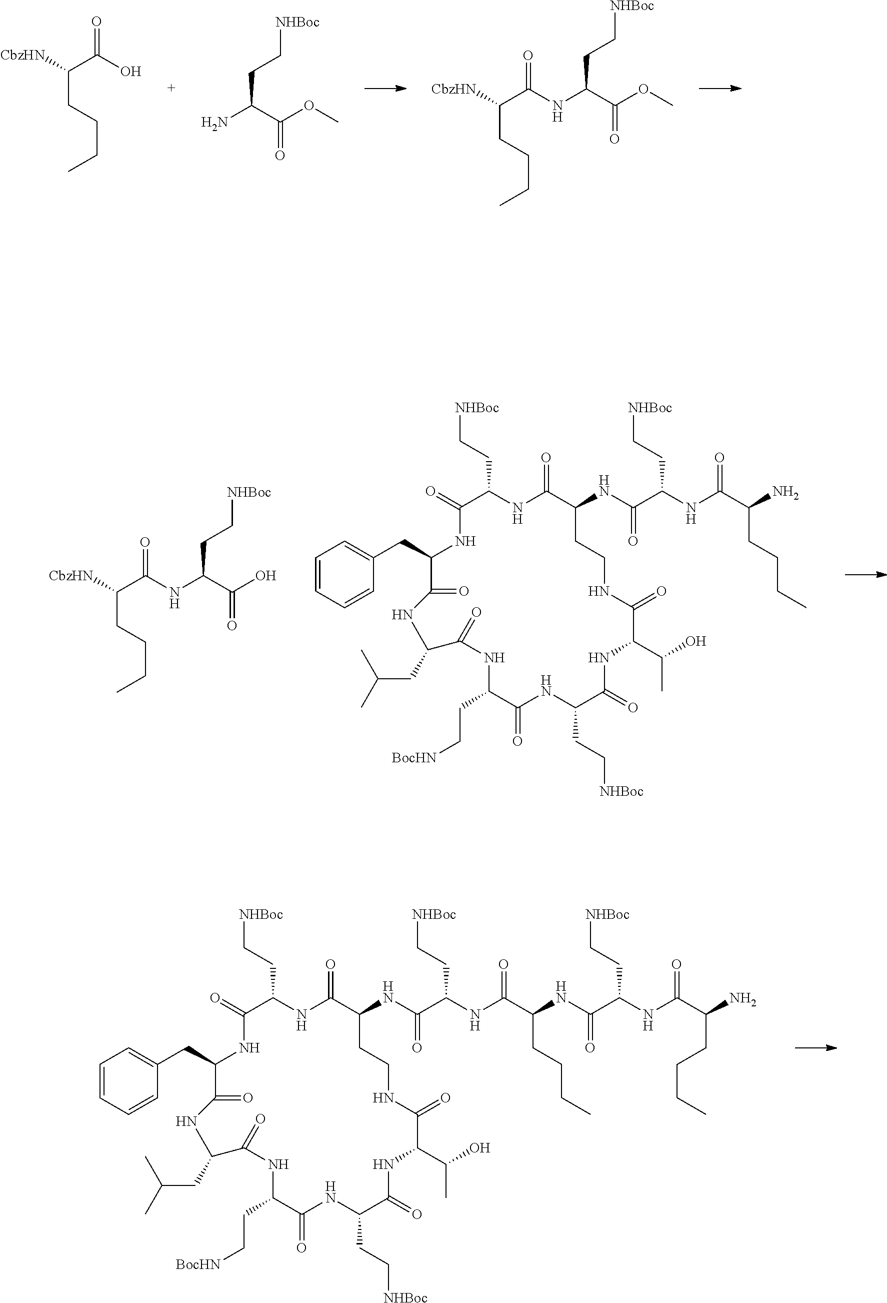

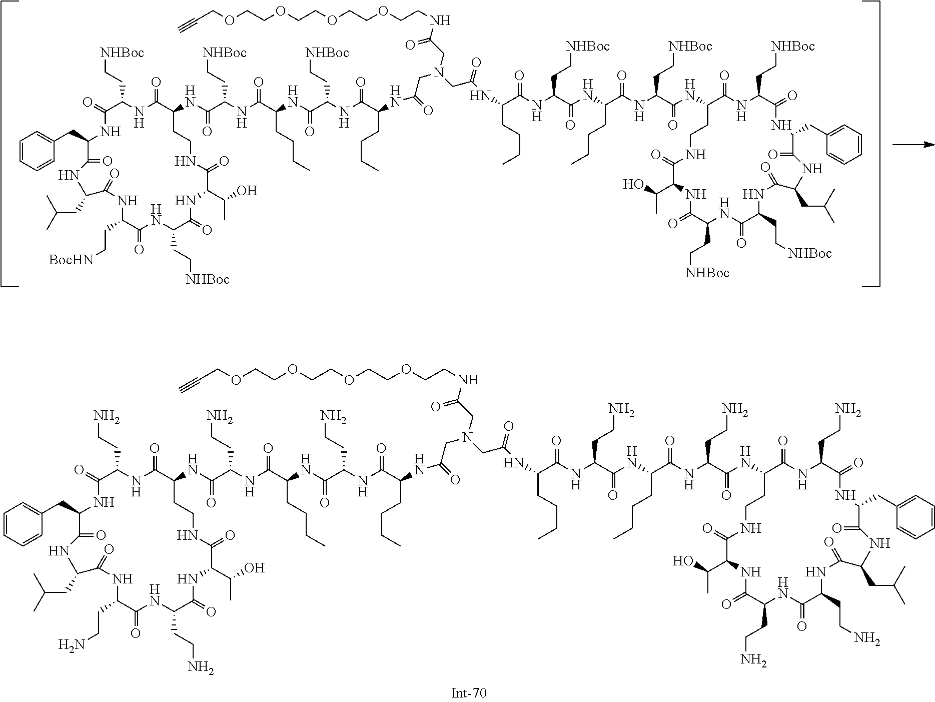

### Step a

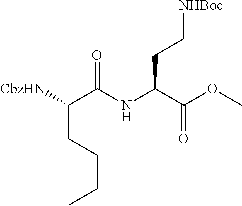 [SMILES: CCCC[C@H](NC(=O)OCc1ccccc1)C(=O)N[C@@H](CCNC(=O)OC(C)(C)C)C(=O)OC]

To a solution of Z-L-norleucine (1.062 g, 4 mmol) in anhydrous NMP (5 mL) was added (S)-methyl 2-amino-4-((tert-butoxycarbonyl)amino)butanoate HCl salt (1.182 g, 4.4 mmol) and DIPEA (1.14 g, 8.8 mmol). The resulting mixture was treated with HATU (760.4 mg, 2.0 mmol) in portions over 10 minutes, and then stirred for 1 hour. It was then purified by RPLC (100 g column, 20 to 75% acetonitrile and water). Yield 1.9 g, 99.4%. Ion found by LCMS: [M-Boc+H]⁺=380.2.

### Step b

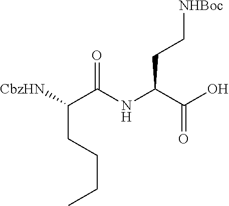 [SMILES: CCCC[C@H](NC(=O)OCc1ccccc1)C(=O)N[C@@H](CCNC(=O)OC(C)(C)C)C(=O)O]

The step-a product (1.9 g, 3.975 mmol) was dissolved in MeOH (5 mL) and THF (10 mL). After the solution was cooled in an ice-water bath, it was treated with a solution of LiOH monohydrate (250 mg, 6 mmol) in water (10 mL). After the reaction was stirred for 30 minutes, DCM (100 mL) was added, and the reaction was continued stirring for 30 minutes. It was then acidified with 4N HCl solution in dioxane (1.5 mL), and diluted with water (30 mL). Two layers were separated, and the aqueous layer was back-extracted with EtOAc (80 mL×2). The combined organic layers were dried over Na₂SO₄, concentrated by rotary evaporation, and further dried under high vacuum. Yield 1.72 g, 93%. Ion found by LCMS: [M−Boc+H]⁺=366.

### Step c

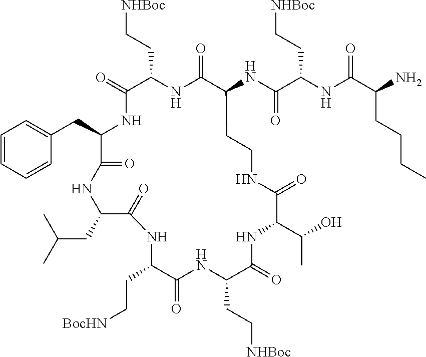 [SMILES: CCCC[C@H](N)C(=O)N[C@@H](CCNC(=O)OC(C)(C)C)C(=O)N[C@H]1CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](Cc2ccccc2)NC(=O)[C@H](CCNC(=O)OC(C)(C)C)NC1=O]

To a solution of the step-b product (535.9 mg, 1.2 mmol) and PMB heptapeptide (1.06 g, 1 mmol, Example 2) in anhydrous DMF (2 mL) was added HATU (456.2 mg, 1.2 mmol) in portions over 10 minutes, followed by DIPEA (312 mg, 2.4 mmol). After the reaction was stirred for 1 hour, it was purified by RPLC (100 g, 40 to 95% MeOH and water). The collected fractions were concentrated by rotary evaporation to a white solid (Ion found by LCMS: [(M−2Boc+2H)/2]⁺=655.6). The material was re-dissolved in MeOH (30 mL) and treated with Pd/C (5%). The mixture was stirred at room temperature overnight. Pd/C was filtered, and the filtrate was concentrated by rotary evaporation to dryness. Yield 1.177 g, 85% over two steps. Ion found by LCMS: [(M−Boc+2H)/2]⁺=638.4.

### Step d

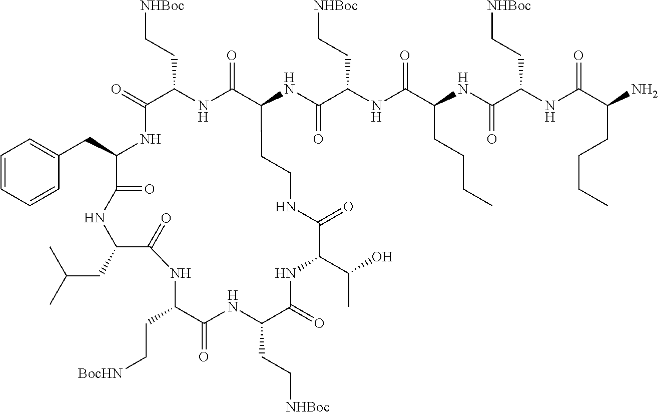 [SMILES: CCCC[C@H](NC(=O)[C@H](CCNC(=O)OC(C)(C)C)NC(=O)[C@@H](N)CCCC)C(=O)N[C@@H](CCNC(=O)OC(C)(C)C)C(=O)N[C@H]1CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](Cc2ccccc2)NC(=O)[C@H](CCNC(=O)OC(C)(C)C)NC1=O]

To a solution of the step-b product (475.1 mg, 1.02 mmol) and the step-c product (1.177 g, 0.851 mmol) in anhydrous DMF (2 mL) was added HATU (388 mg, 1.02 mmol) in portions over 10 minutes, followed by DIPEA (260 mg, 2 mmol), After the reaction was stirred for 1 hour, it was purified by RPLC (100 g, 40 to 95% MeOH and water). The collected fractions were concentrated by rotary evaporation to a white solid (Ion found by LCMS: [(M−2Boc+2H)/2]⁺=821.1). The material was re-dissolved in MeOH (30 mL) and treated with Pd/C (5%). The mixture was stirred at room temperature overnight. Pd/C was filtered, and the filtrate was concentrated by rotary evaporation to dryness. Yield 1.196 g, 83.3% over two steps. Ion found by LCMS: [(M−Boc+2H)/2]⁺=795.

### Step e

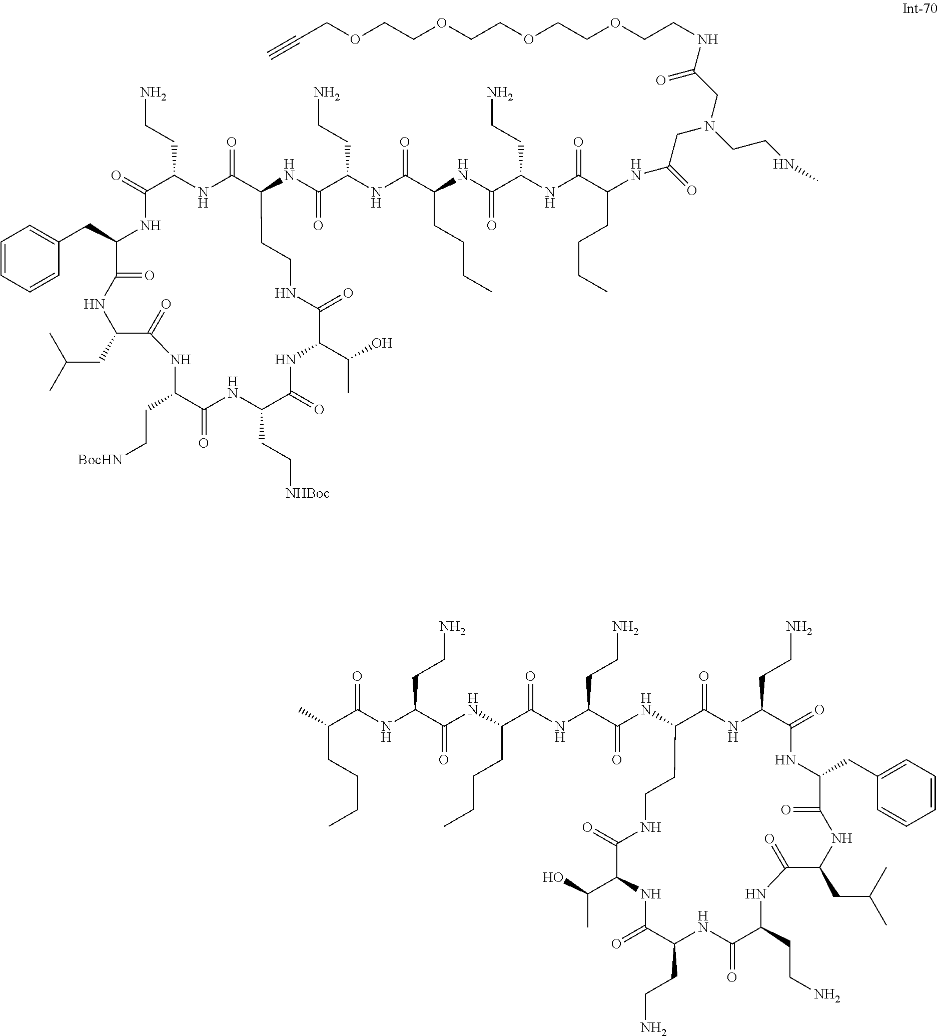

To a mixture of diacid (40.4 mg, 0.1 mmol, described in Example 67, step i) and the step-d product from this example (371.6 mg, 0.22 mmol) in anhydrous DMF (2 mL) was added HATU (76 mg, 0.2 mmol) and DIPEA (52 mg, 0.4 mmol). The resulting mixture was stirred for 30 minutes and was purified by RPLC (50 g, column 40 to 95% MeOH and water, using 0.1% TFA as modifier). The collected fractions were concentrated to a white solid (Ions found by LCMS: [(M−2Boc+3H)/3]⁺=1182.0, [(M−4Boc+3H)/3]⁺=1116.0, [(M−5Boc+3H)/3]⁺=1083. The material was re-dissolved in TFA (0.5 mL) and DCM (˜1 mL). The solution was stirred for 30 minutes and directly purified through RPLC (50 g column, 3 to 50% acetonitrile and water, using 0.1% TFA as modifier). Yield of Int-70 was 102.4 mg, 25.6% over two steps. Ions found by LCMS: [(M+5H)/5]⁺=549.6, [(M+6H)/6]⁺=458.2, [(M+7H)/7]⁺=393.0.

### Example 106—Synthesis of Int-71

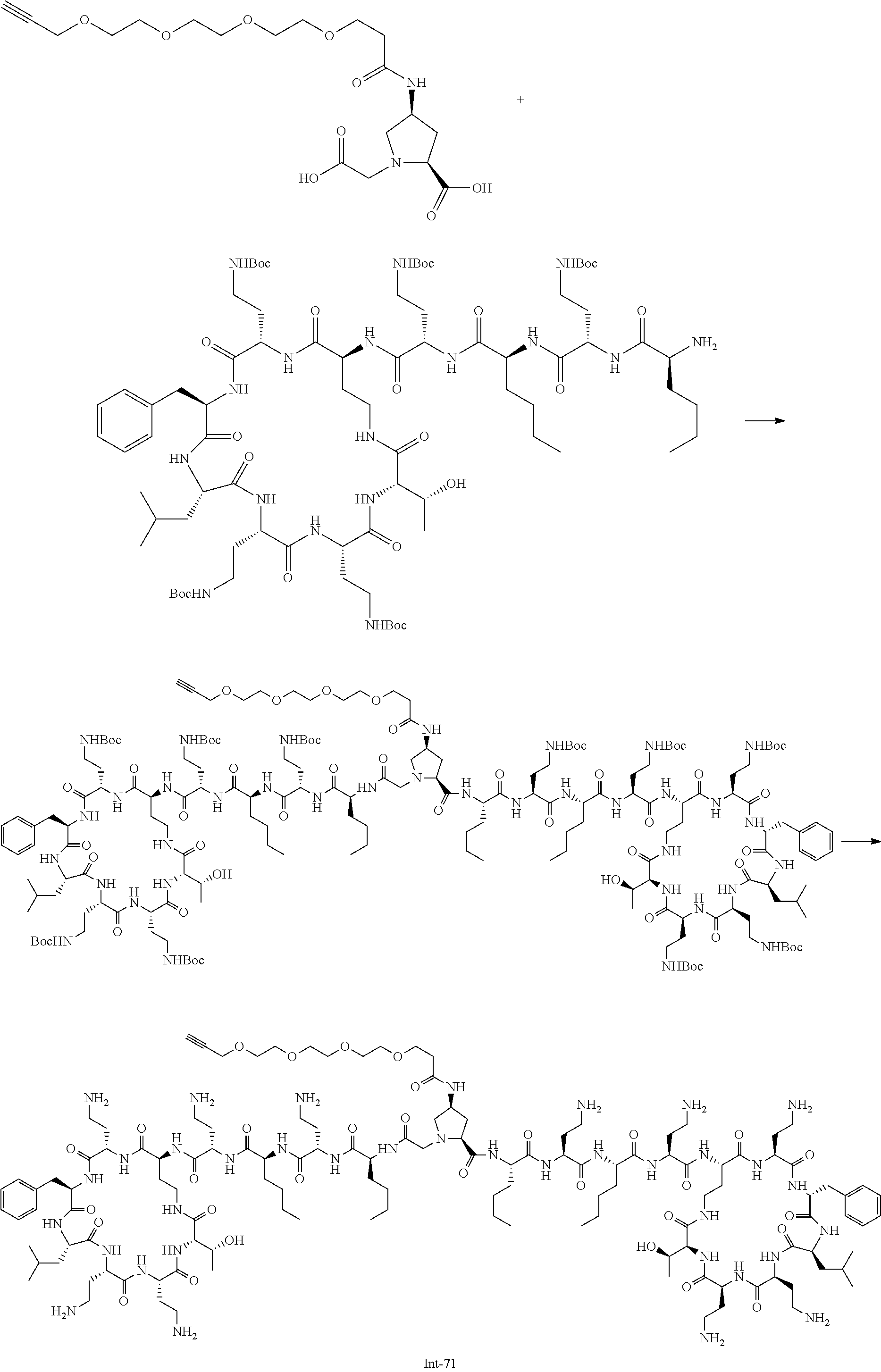

### Step a

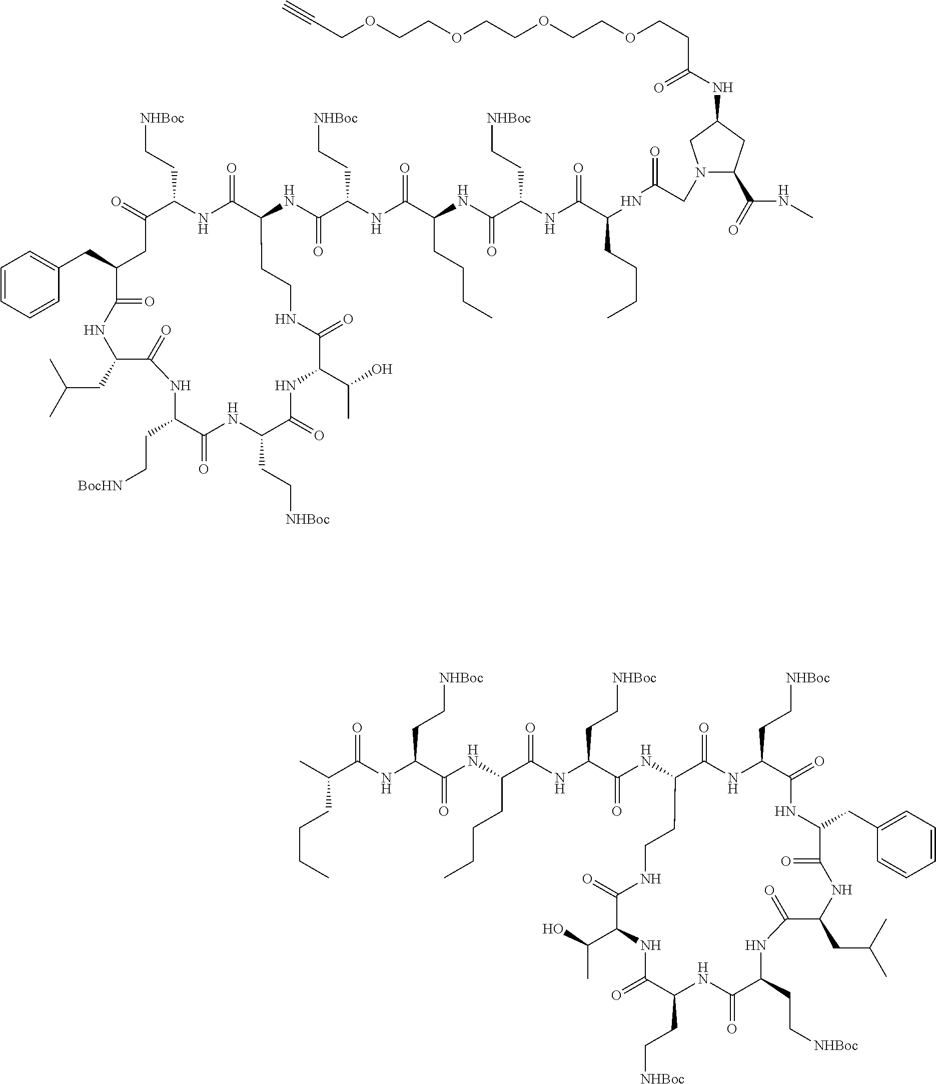

The compound was prepared analogously to Example 101, step-b, using intermediates from Example 105, step-d, and Example 104, step-d. Yield 171 mg, 45.3%. Ions found by LCMS: [(M−2Boc+3H)/3]⁺=1191.0, [(M−3Boc+3H)/3]⁺=1157.6, [(M−5Boc+3H)/3]⁺=1091.2.

### Step b

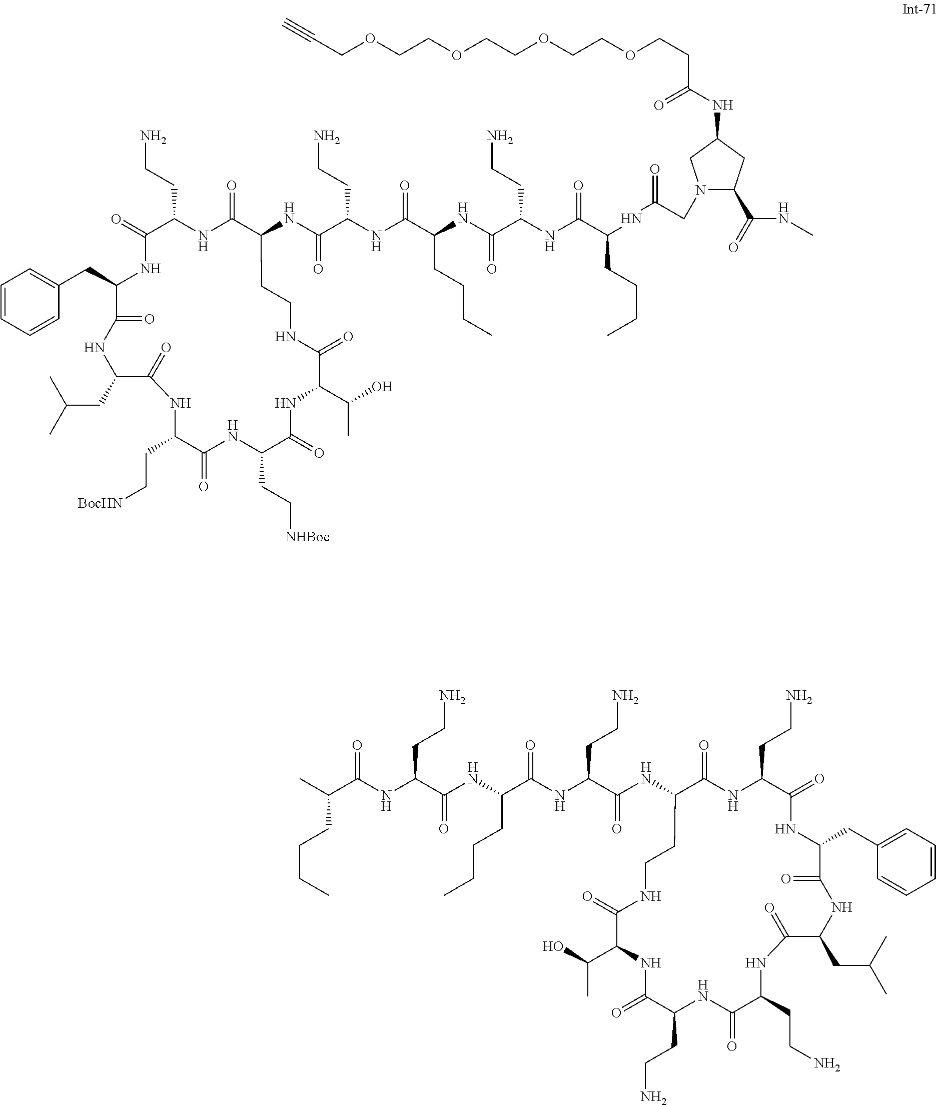

The title compound Int-71 was prepared analogously to Example 101, step-d, using the s ep-a intermediate described in this example. Yield 147 mg, 81.6%. Ions found by LCMS: [(M+5H)/5]⁺=554.9, [(M+6H)/6]⁺=462.6, [(M+7H)/7]⁺=396

### Example 107—Syntheses of Int-72

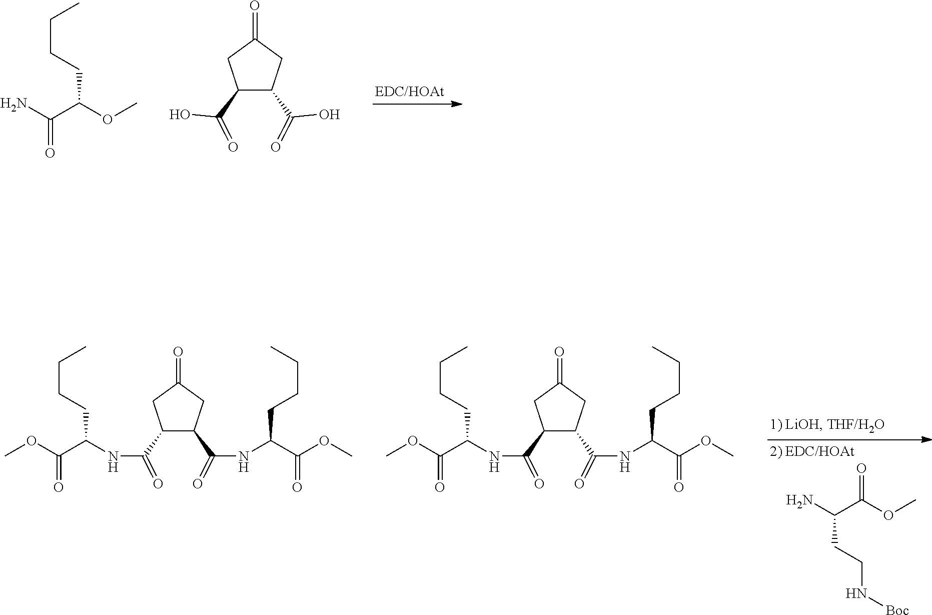

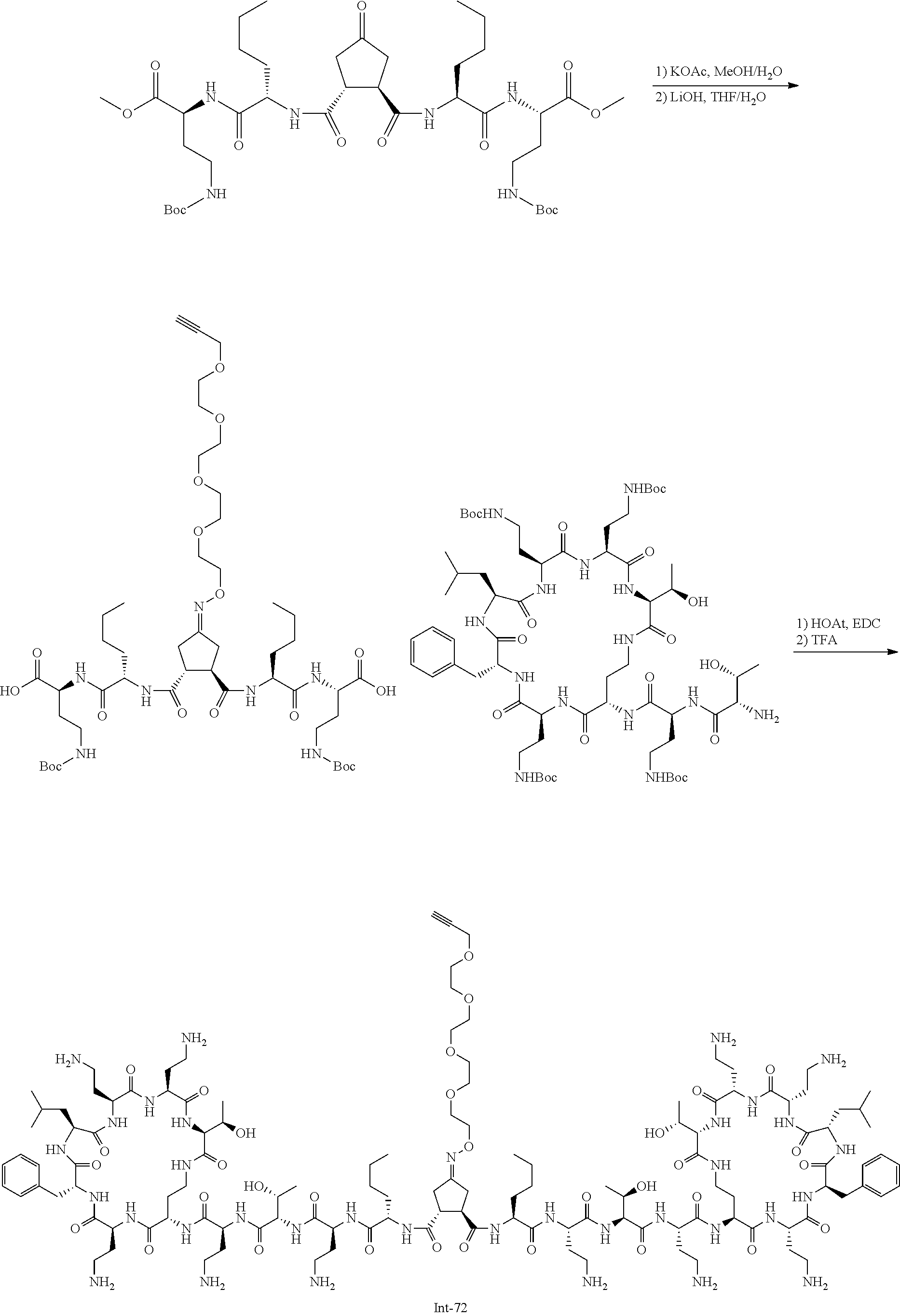

### Step a

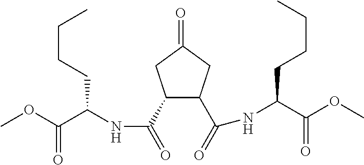

A solid mixture of norleucine methyl ester HCl salt (2.110 g, 11.62 mmol), racemic trans-4-oxocyclopentane-1,2-dicarboxylic acid (1.000 g, 5.81 mmol), EDCI (2.783 g, 14.52 mmol), HOAt (1.9786 g, 14.52 mmol) and NaHCO₃ (1.952 g, 23.23 mmol) were mixed in 20 mL of DCM/DMF (5:1). The mixture was stirred for overnight (˜15 hours) at room temperature. LC/MS analysis showed the completion of the reaction. Two diastereomers were separated by reversed phase HPLC. The polar diastereomer and less polar diastereomer have the retention time at 4.83 min. and 5.04 min. respectively with 8-minute 5˜95% ACN/water method: (M+H)⁺=427.0 and M−H⁺=425.0. The reaction mixture was diluted with EtOAc (200 mL) and washed with 1 N aq. HCl, saturated NaHCO₃ and brine respectively. The solution was dried with anhy. Sodium sulfate and the solvents were removed. The residue was dissolved in minimum amount of NMP and purified by RPLC using an Isco CombiFlash liquid chromatograph eluted with 30-60% acetonitrile and water. The pure polar diastereomer dimethyl ester, 1.071 g, was obtained in 46% yield (50% in theory). The absolute stereochemistry of the polar and less polar isomers was determined by X-ray. The polar isomer was used in the next step.

### Step b

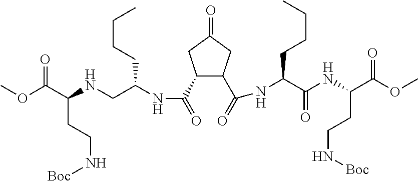

The polar dimethyl ester (1.071 g, 2.511 mmol) was treated with LiOH (0.1302 g, 5.273 mmol) in 20 mL of THF/Water (1:1). Stirred for less than 1 hrs. 1N HCl to acidify. Removed THF and extracted with EtOAc to give diacid (1.000 g, 100%). Positive mass ions was found as (M+H⁺): 399.0, tr=1.3 minutes with 5-minute 40˜95% ACN/water method.

A solid mixture of diacid (1.000 g, 2.510 mmol), Dab(□-Boc) methyl ester HCl salt (1.349 g, 5.020 mmol), EDCI (1.203 g, 6.274 mmol), HOAt (0.8540 g, 6.274 mmol) and NaHCO₃ (0.8424 g, 10.04 mmol) were mixed in 20 mL of DCM/DMF (5:1). The mixture was stirred for overnight (˜15 hours) at room temperature. LC/MS analysis showed the completion of the reaction. The reaction mixture was diluted with EtOAc (200 mL) and washed with 1 N aq. HCl, saturated NaHCO₃ and brine respectively. The solution was dried with anhy. Sodium sulfate and the solvents were removed. The residue was dissolved in minimum amount of NMP and purified by RPLC using an Isco CombiFlash liquid chromatograph eluted with 30-60% acetonitrile and water. The cyclopentanonyl central linker, 1.760 g, was obtained in 85% yield. Ions found by LCMS: (M−2Boc+2H⁺)/2=314.0.

### Step c

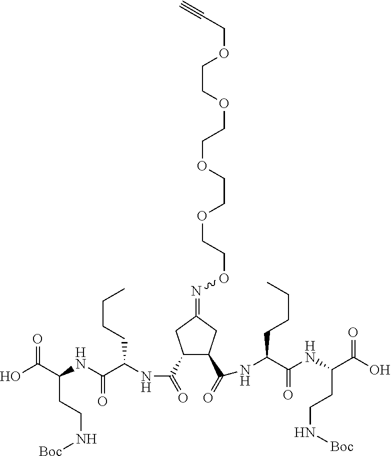 [SMILES: C#CCOCCOCCOCCOCCON=C1C[C@@H](C(=O)N[C@@H](CCCC)C(=O)N[C@@H](CCNC(=O)OC(C)(C)C)C(=O)O)[C@H](C(=O)N[C@@H](CCCC)C(=O)N[C@@H](CCNC(=O)OC(C)(C)C)C(=O)O)C1]

A mixture of cyclopentanonyl central linker (0.8360 g, 1.011 mmol), aminooxy-PEG4-propargyl (0.500 g, 2.022 mmol), and potassium acetate (0.2977 g, 3.033 mmol) were dissolved in 20 mL of methanol/water (5:1). The mixture was stirred for overnight (˜15 hours) at 60° C. HPLC analysis showed the completion of the reaction. Most of the solvents of the reaction mixture was removed by reduced pressure rotovap to give a viscous oily residue. The residue was dissolved in minimum amount of NMP and purified by RPLC using an Isco CombiFlash liquid chromatograph eluted with 30-60% acetonitrile and water. The pure PEG4-oxime central linker dimethyl ester, 1.0457 g, was obtained in 98% yield. The dimethyl ester has the retention time at 5.47 min. with 8-minute 5-95% ACN/water method: (M−2Boc+2H⁺)/2=428.6.

The dimethyl ester (1.0457 g, 0.9900 mmol) was treated with LiOH (0.0.0513 g, 2.079 mmol) in 10 mL of THF/Water (1:1). Stirred for less than 1 hrs. 1N HCl to acidify. Removed THF and extracted with EtOAc to give PEG4-oxime central linker (1.011 g, 99%). Positive mass ions was found as (M−2Boc+2H⁺)/2=414.6, tr=4.50 minutes with 8-minute 5˜95% ACN/water method.

### Step d

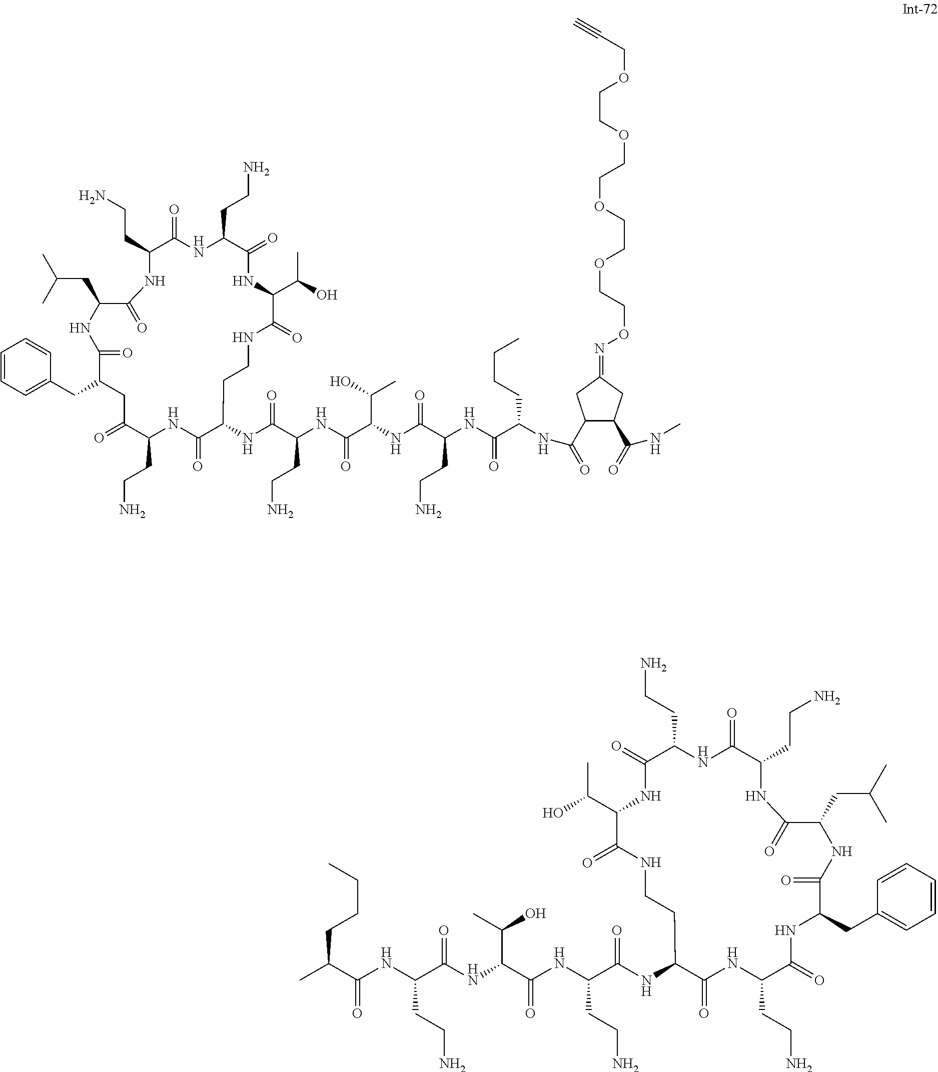

A solid mixture of Int-7a (0.6631 g, 0.4863 mmol, Example 8, step a), PEG4-oxime central linker (0.250 g, 0.2432 mmol), EDCI (0.1165 g, 0.6079 mmol) and HOAt (0.0827 g, 0.608 mmol) were dissolved in 5 mL of dry DMF immediately followed by adding triethylamine (0.17 mL, 0.97 mmol). The mixture was stirred for overnight (˜15 hours) at room temperature. HPLC analysis showed the completion of the reaction. Most of the solvent (DMF) of the reaction mixture was removed by reduced pressure rotovap to give a viscous oily residue. The residue was triturated with methanol. After some crystalline solid started to precipitate the flask was put standstill for more than 3 hours. Simple filtration collected the crystalline white solid with methanol wash (3×5 mL). The solid was dried to give 0.675 grams of pure product, 75% isolated yield. The solid (0.440 g) was dissolved with 100% TFA and stirred for 30 minutes. TFA was removed by rotovap. The residue was dissolved in minimum amount of water and purified by RPLC using an Isco CombiFlash liquid chromatograph eluted with 5% to 35% acetonitrile and water using 0.1% TFA modifier. The pure fractions were lyophilized to afford the product Int-72 as a white powder (TFA salt, 0.360 g, 65% isolated yield, Positive mass ions were found by LC/MS: (M+6H⁺)/6=454.0, (M+5H⁺)/5=544.6, (M+4H⁺)/4=680.4.

### Example 108—Syntheses of Int-73

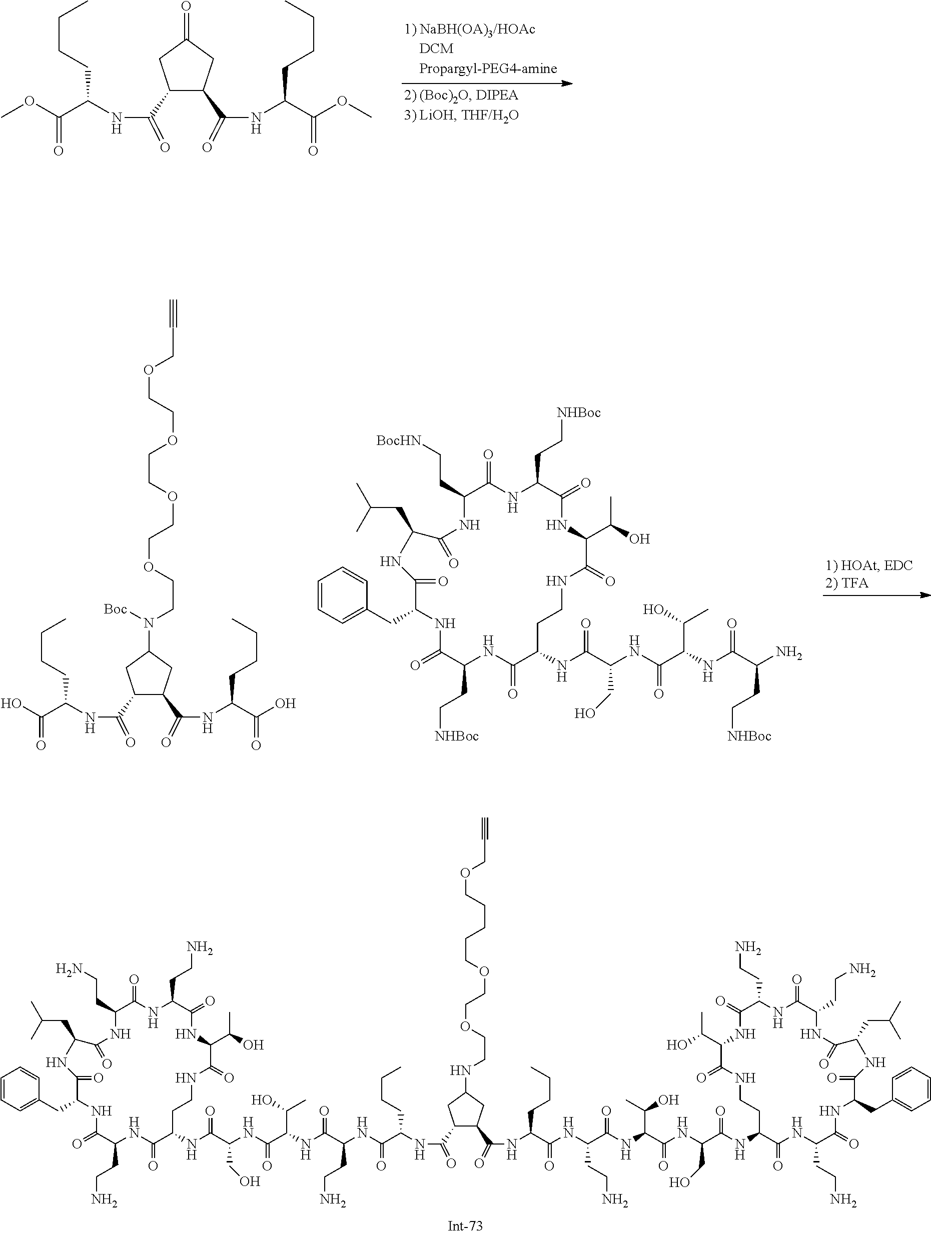

### Step a

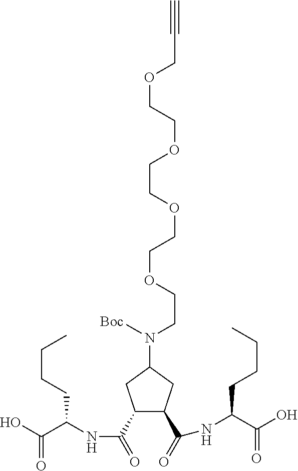 [SMILES: C#CCOCCOCCOCCOCCN(C(=O)OC(C)(C)C)C1C[C@@H](C(=O)N[C@@H](CCCC)C(=O)O)[C@H](C(=O)N[C@@H](CCCC)C(=O)O)C1]

A mixture of the polar dimethyl ester (from Synthesis of, Step 1, 0.9959 g, 2.335 mmol), aminooxy-PEG4-propargyl (0.6745 g, 2.916 mmol) and acetic acid (0.26 mL, 4.5 mmol) were dissolved in 20 mL of DCM. The mixture was stirred for 1 hour at room temperature followed by addition of sodium triacetatoxyborohydride (0.9509 g, 4.486 mmol). The reaction mixture was stirred for overnight (˜15 hours) at room temperature. HPLC analysis showed the completion of the reaction. Positive mass ion was found by LC/MS: (M+H)⁺=642.4. Most of the solvents of the reaction mixture was removed by reduced pressure rotovap to give a viscous oily residue. The residue was dissolved in 20 mL of THF/10% NaHCO₃ (1:1) and treated with Boc anhydride (0.71 mL, 3.1 mmol) and diisopropylethylamine (0.54 mL, 3.1 mmol). The mixture was stirred for overnight (˜15 hours) at room temperature. Most of THF was removed and the residue was extracted with EtOAc. After removal of EtOAc, the residue was dissolved in minimum amount of NMP and purified by RPLC using an Isco CombiFlash liquid chromatograph eluted with 30-60% acetonitrile and water. The pure PEG4-amino cyclopentanyl dimethyl ester, 0.7405 g, was obtained in 65% yield. The dimethyl ester has the retention time at 2.88 min. with 5-minute 40˜95% ACN/water method: (M+H)⁺=742.4 and M-Boc+H⁺=643.0.

The dimethyl ester (0.7405 g, 0.9980 mmol) was treated with LiOH (0.0.0616 g, 2.495 mmol) in 10 mL of THF/Water (1:1). Stirred for less than 1 hrs then acidified with 1N HCl. The solution was concentrated and extracted with EtOAc to give Boc-PEG4-central linker (0.705 g, 99%). Positive mass ions was found as (M+H)⁺=714.4, tr=3.30 minutes with 5-minute 5˜95% ACN/water method.

### Step b

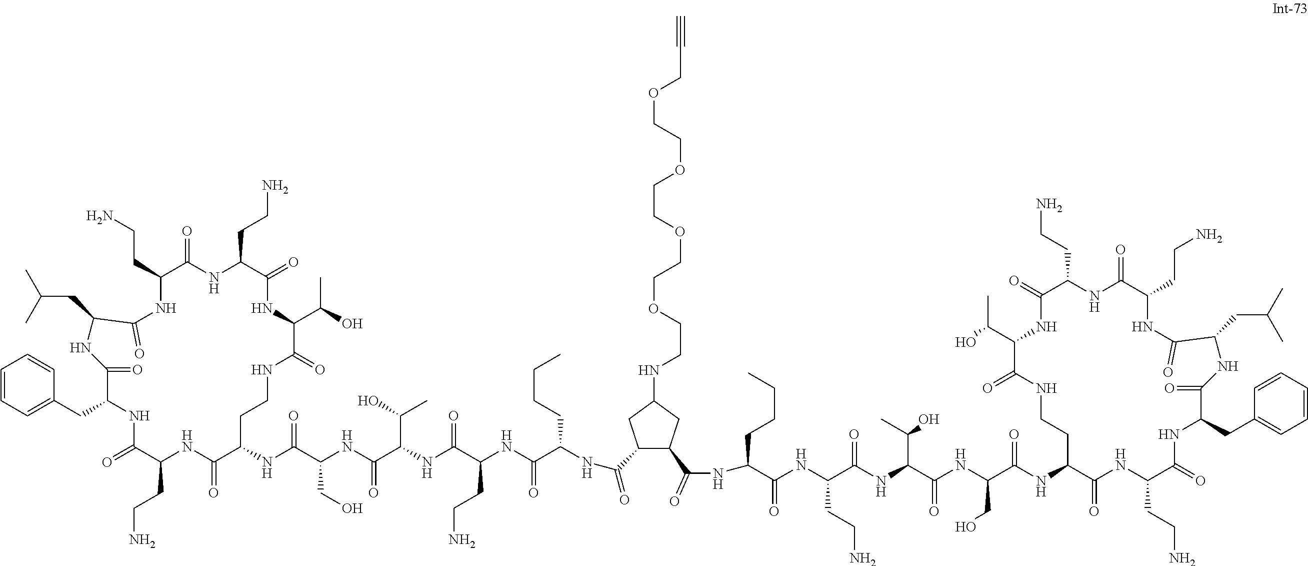 [SMILES: C#CCOCCOCCOCCOCCNC1C[C@@H](C(=O)N[C@@H](CCCC)C(=O)N[C@@H](CCN)C(=O)N[C@H](C(=O)N[C@H](CO)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)[C@H](C(=O)N[C@@H](CCCC)C(=O)N[C@@H](CCN)C(=O)N[C@H](C(=O)N[C@H](CO)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)C1]

A solid mixture of Int-5d (0.6544 g, 0.4511 mmol), Boc-PEG4 central linker (0.1610 g, 0.2255 mmol), EDCI (0.1081 g, 0.5636 mmol) and HOAt (0.07675 g, 0.5638 mmol) were dissolved in 5 mL of dry DMF immediately followed by adding triethylamine (0.16 mL, 0.90 mmol). The mixture was stirred for overnight (˜15 hours) at room temperature. HPLC analysis showed the completion of the reaction. Most of the solvent (DMF) of the reaction mixture was removed by reduced pressure rotovap to give a viscous oily residue. To this residue methanol was added with gentle stirring to triturate the reaction product. After some crystalline solid started to precipitate the flask was put standstill for more than 3 hours. Simple filtration collected the crystalline white solid with methanol wash (3×5 mL). The solid was dried to give 0.607 grams of pure product, 75% isolated yield. Positive ions found: (M−3Boc+3H⁺)/3=1093.8. The solid (0.440 g) was dissolved with 100% TFA and stirred for 30 minutes. TFA was removed by rotovap. The residue was dissolved in minimum amount of water and purified by RPLC using an Isco CombiFlash liquid chromatograph eluted with 5% to 35% acetonitrile and water using 0.1% TFA modifier. The pure fractions were lyophilized to afford the desired product Int-73 as a white powder (TFA salt, 0.670 g, 87% isolated yield). Positive ions found by LC/MS: (M+7H⁺)/7=383.4, (M+6H⁺)/6=447.2. (M+5H⁺)/5=536.2. (M+4H⁺)/4=670.1.

### Example 109—Synthesis of Conjugate 27

A solution of azido-hIgG1(Myc)Fc (50 mg, 0.85 μmol, 15.9 mg/mL, MW=58,743 Da, DAR=3.3) in pH 7.4 PBS was treated with freshly prepared PBS 7.4 solutions of alkyne derivatized small molecule (37.0 mg, 25.5 μmol, Example 38), THPTA (5.5 mg, 12.8 μmol, tris(3-hydroxypropyltriazolylmethyl)amine), sodium ascorbate (5.0 mg, 25.5 μmol), and CuSO₄ (2.0 mg, 12.8 μmol). The resulting solution was agitated on a rocker table overnight at room temperature, then purified by affinity chromatography over a protein-A column (See Example 164). Maldi TOF analysis of the purified final product gave an average mass of 63,234 Da (DAR=3.1). Yield 33 mg, 61% yield.

### Example 110—Synthesis of Conjugate 38

A solution of azido-hIgG1(Myc)Fc (30 mg, 0.50 μmol, 15.9 mg/mL, MW=58,743 Da, DAR=4.0) in pH 7.4 PBS was treated with freshly prepared PBS 7.4 solutions of alkyne derivatized small molecule (52.0 mg, 12.6 μmol, See Example 59. Synthesis of int-24), THPTA (3.3 mg, 7.5 μmol, tris(3-hydroxypropyltriazolylmethyl)amine), sodium ascorbate (3.0 mg, 15.1 μmol), and CuSO₄ (1.2 mg, 7.5 μmol). The resulting solution was agitated on a rocker table overnight at room temperature, then purified by affinity chromatography over a protein-A column (See Example 164). Maldi TOF analysis of the purified final product gave an average mass of 67,320 Da (DAR=3.8 calculated from the tallest peak). Yield 17.8 mg, 59% yield.

### Example 111—Synthesis of Conjugate 39

A solution of azido-hIgG1(Myc)Fc (30 mg, 0.50 μmol, 15.9 mg/mL, MW=59,641 Da, DAR=4.0) in pH 7.4 PBS was treated with freshly prepared PBS 7.4 solutions of alkyne derivatized small molecule (43.4 mg, 12.6 μmol. See Example 60), THPTA (3.3 mg, 7.5 μmol, tris(3-hydroxypropyltriazolylmethyl)amine), sodium ascorbate (3.0 mg, 15.1 μmol), and CuSO₄ (1.2 mg, 7.5 μmol). The resulting solution was agitated on a rocker table overnight at room temperature, then purified by protein-A affinity chromatography, followed by size exclusion chromatography (See Example 164 for purification procedure). Maldi TOF analysis of the purified final product gave an average mass of 68,535 Da (DAR=3.8 calculated from a weighted average of the Maldi peaks). Yield 16.7 mg, 48% yield.

### Example 112—Synthesis of Conjugate 40

A solution of azido-hIgG1(Myc)Fc (30 mg, 0.50 μmol, 15.9 mg/mL, MW=59,641 Da, DAR=4.0) in pH 7.4 PBS was treated with freshly prepared PBS 7.4 solutions of alkyne derivatized small molecule (46.3 mg, 12.6 μmol, See Example 61), THPTA (3.3 mg, 7.5 μmol, tris(3-hydroxypropyltriazolylmethyl)amine), sodium ascorbate (3.0 mg, 15.1 μmol), and CuSO₄ (1.2 mg, 7.5 μmol). CuSO₄ was added last and caused formation of a cloudy white mixture. The resulting solution was agitated on a rocker table overnight at room temperature, then purified by protein-A affinity chromatography, followed by size exclusion chromatography (See Example 164 for purification procedure). Maldi TOF analysis of the purified final product gave an average mass of 69,074 Da (DAR=3.7 calculated from a weighted average of the Maldi peaks). Yield 12.3 mg, 41% yield.

### Example 113—Synthesis of Conjugate 74

A solution of hIgG1(Myc)Fc (30 mg, 0.51 μmol, 15.9 mg/mL, MW=58,270 Da) in pH 7.4 PBS was treated with TCEP (18.5 μL, 0.5 M, 9.3 μmol) at room temperature. LCMS after 1 h showed complete reduction of the disulfides. This solution was buffer exchanged with PBS, pH=7.8, EDTA 2 mM (3×15 mL) using a 10,000 mw cutoff filer (final volume 4 mL). To this was added a freshly prepared solution of azido-PEG-4-bissulfone linker (1.44 mg, 20.6 μmol, in 0.2 mL DMF) resulting in a cloudy solution.

### Azido-PEG-4-Bissulfone Linker:

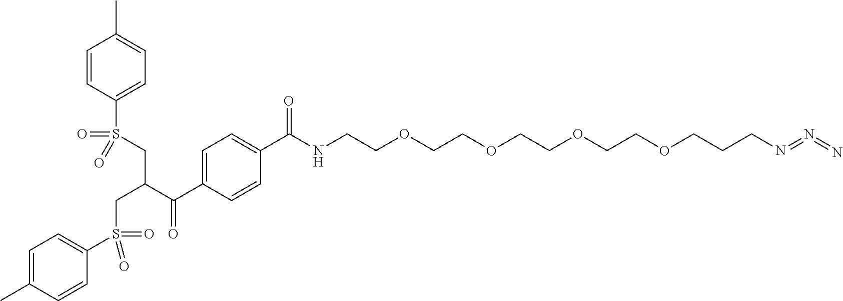

Addition of 1 mL methanol removed the cloudiness. The resulting reaction was agitated overnight on a rocker table at room temperature. The azido Fc was purified by protein-A affinity chromatography (See Example 164). LCMS showed clean incorporation of two bissulfone linker units (mass=59,050, azide DAR=2.0).

A solution of cysteine-azido-hIgG1(Myc)Fc (28.6 mg, 0.48 μmol, 11.7 mg/mL, DAR=2.0) in pH 7.4 PBS was treated with freshly prepared PBS 7.4 solutions of alkyne derivatized small molecule (22.4 mg, 5.8 μmol, See Example 41, INT-21), THPTA (2.1 mg, 4.8 μmol, tris(3-hydroxypropyltriazolylmethyl)amine), sodium ascorbate (2.9 mg, 14.5 μmol), and CuSO₄ (0.77 mg, 4.8 μmol). CuSO₄ was added last and caused formation of a slightly cloudy solution. The resulting solution was agitated on a rocker table overnight at room temperature, then purified by protein-A affinity chromatography, followed by size exclusion chromatography (See Example 164 for purification procedure). Maldi TOF analysis of the purified final product gave an average mass of 64,485 Da (DAR=3.7 calculated from a weighted average of the Maldi peaks). Yield 16 mg, 52% yield.

### Example 114—Synthesis of Conjugate 75

A solution of azido-hIgG1(Myc)Fc (30 mg, 0.50 μmol, 15.9 mg/mL, MW=59,641 Da, DAR=2.8) in pH 7.4 PBS was treated with freshly prepared PBS 7.4 solutions of alkyne derivatized small molecule (15.8 mg, 5.6 μmol, See Example 62), THPTA (3.3 mg, 7.5 μmol, tris(3-hydroxypropyltriazolylmethyl)amine), sodium ascorbate (3.0 mg, 15.1 μmol), and CuSO₄ (1.2 mg, 7.5 μmol). CuSO₄ was added last and caused formation of a cloudy white mixture. The resulting solution was agitated on a rocker table overnight at room temperature, then purified by protein-A affinity chromatography, followed by size exclusion chromatography (See Example 164 for purification procedure). Maldi TOF analysis of the purified final product gave an average mass of 69,074 Da DAR=3.7 calculated from a weighted average of the Maldi peaks). Yield 12.3 mg. 41% yield.

### Example 115—Synthesis of Conjugate 28

To a room temperature solution of alkyne functionalized small molecule (Example 64, 0.050 mg, 13.5 μmol) in pH 7.4 PBS (500 μL), was added in order a 10 mM solution of THPTA in pH 7.4 PBS (700 μL, 7.0 μmol), a 20 mM solution of sodium ascorbate in pH 7.4 PBS (700 μL, 14.0 μmol), and last a 10 mM solution of copper sulfate in water (700 μL, 7.0 μmol). Upon mixing, a solution of azido-hIgG1(Myc)Fc (29.4 mg, 0.500 μmol, 10 mg/mL, MW=58,837.00 Da, DAR=2.7) in pH 7.4 PBS was gently dispersed. The resulting solution was agitated on a rocker table overnight at room temperature, then purified by affinity chromatography over a protein-A column (See Example 164). Maldi TOF analysis of the purified final product gave an average mass of 63,812 Da (DAR=2.4). Yield 10.35 mg, 52% yield.

### Example 116—Synthesis of Conjugate 29

To a room temperature solution of material described in Example 63 (0.047 mg, 13.5 μmol) in pH 7.4 PBS (500 μL), was added in order: a 10 mM solution of THPTA in pH 7.4 PBS (700 μL, 7.0 μmol), a 20 mM solution of sodium ascorbate in pH 7.4 PBS (700 μL, 14.0 μmol), and last a 10 mM solution of copper sulfate in water (700 μL, 7.0 μmol). Upon mixing, a solution of azido-hIgG1(Myc)Fc (29.4 mg, 0.500 μmol, 10 mg/mL, MW=58,837.00 Da, DAR=2.7) in pH 7.4 PBS was gently dispersed. The resulting solution was agitated on a rocker table overnight at room temperature, then purified by affinity chromatography over a protein-A column (See Example 164). Maldi TOF analysis of the purified final product gave an average mass of 60,951 Da (DAR=1.6). Yield 15.75 mg, 78% yield.

### Example 117—Synthesis of Conjugate 55

To a room temperature solution of alkyne functionalized small molecule (Example 65, 0.054 mg, 13.6 μmol) in pH 7.4 PBS (500 μL), was added in order a 20 mM solution of THPTA in pH 7.4 PBS (340 μL, 6.8 μmol), a 20 mM solution of sodium ascorbate in pH 7.4 PBS (2,720 μL, 54.4 μmol), and last a 20 mM solution of copper sulfate in water (340 μL, 6.8 μmol). Upon mixing, the solution of azido-hIgG1(Myc)Fc (29.4 mg, 0.340 μmol, 10 mg/mL, MW=58,821.00 Da, DAR=2) in pH 7.4 PBS was gently dispersed. The resulting solution was agitated on a rocker table overnight at room temperature, then purified by affinity chromatography over a protein-A column (See Example 164). Maldi TOF analysis of the purified final product gave an average mass of 61,939 Da (DAR=1.1). Yield 5.92 mg, 30% yield.

### Example 118—Synthesis of Conjugate 76

To a room temperature solution of alkyne functionalized small molecule (Example 66, 0.056 mg, 10.2 μmol) in pH 7.4 PBS (500 μL), it was added in the order a 20 mM solution of THPTA in pH 7.4 PBS (340 μL, 6.8 μmol), a 20 mM solution of sodium ascorbate in pH 7.4 PBS (680 μL, 13.6 μmol), and last a 20 mM solution of copper sulfate in water (340 μL, 6.8 μmol). Upon mixing, a solution of azido-hIgG1(Myc)Fc (0.020 mg, 0.340 μmol, 10 mg/mL, MW=58,833.00 Da, DAR=3) in pH 7.4 PBS was gently dispensed. The resulting solution was agitated on a rocker table overnight at room temperature, then purified by affinity chromatography over a protein-A column (See Example 164). Maldi TOF analysis of the purified final product gave an average mass of 63,911 Da (DAR=1.7). Yield 10.81 mg, 54% yield.

### Example 119—Synthesis of Conjugate 69

A solution of azido-hIgG1(Myc)Fc in PBS buffer (20 mg, 0.34 μmol, 1.40 mL, MW=58,721 Da, DAR=2.7) was treated with alkyne derivatized small molecule (TFA salt, 36 mg, 9.2 μmol, Example 67) and freshly prepared pH 7.4 PBS solutions of CuSO₄ (0.7 mL of 10.0 mM, 20 eq), tris(3-hydroxypropyltriazolylmethyl)-amine (THPTA, 0.7 mL of 10.0 mM, 20 eq), and sodium ascorbate (1.02 mL of 10.0 mM, 30 eq). The resulting solution was agitated gently for 12 hours at room temperature. The reaction mixture was purified by affinity chromatography over a protein A column, followed size exclusion chromatography (See Example 164). MALDI TOF analysis of the purified final product gave an average mass of 64,845 Da (DAR=2.3). Yield 9.36 mg, 67% yield.

### Example 120—Synthesis of Conjugate 34

A solution of azido-hIgG1(Myc)Fc in PBS buffer (30 mg, 0.50 μmol, 2.0 mL, MW=58,548 Da, DAR=2.7) was treated with alkyne derivatized small molecule (TFA salt, 54 mg, 13.5 μmol, Example 68) and freshly prepared pH 7.4 PBS solutions of CuSO₄ (1.0 mL of 10.0 mM, 20 eq), tris(3-hydroxypropyltriazolylmethyl)-amine (THPTA, 1.0 mL of 10.0 mM, 20 eq), and sodium ascorbate (1.4 mL of 10.0 mM, 30 eq). The resulting solution was agitated gently for 12 hours at room temperature. The reaction mixture was purified by affinity chromatography over a protein A column, followed size exclusion chromatography (See Example 164). MALDI TOF analysis of the purified final product gave an average mass of 64.717 Da (DAR=2.26). Yield 12.7 mg, 42% yield.

### Example 121—Synthesis of Conjugate 43

A solution of azido-hIgG1(Myc)Fc in PBS buffer (30 mg, 0.50 μmol, 2.0 mL, MW=58,935 Da, DAR=5) was treated with alkyne derivatized small molecule (TFA salt, 60 mg, 15 μmol, Example 69) and freshly prepared pH 7.4 PBS solutions of CuSO₄ (1.0 mL of 10.0 mM, 20 eq), tris(3-hydroxypropyltriazolylmethyl)-amine (THPTA, 1.0 mL of 10.0 mM, 20 eq), and sodium ascorbate (1.5 mL of 10.0 mM, 30 eq). The resulting solution was agitated gently for 12 hours at room temperature. The reaction mixture was purified by affinity chromatography over a protein A column, followed size exclusion chromatography (See Example 164). MALDI TOF analysis of the purified final product gave an average mass of 70,947 Da (DAR=4.4). Yield 27 mg, 90% yield

### Example 122—Synthesis of Conjugate 78

A solution of azido-hIgG1(Myc)Fc in PBS buffer (20 mg, 0.34 μmol, 1.78 mL, MW=58,833 Da, DAR=3) was treated with alkyne derivatized small molecule (TFA salt, 41 mg, 15 μmol, Example 70) and freshly prepared pH 7.4 PBS solutions of CuSO₄ (0.68 mL of 10.0 mM, 20 eq), tris(3-hydroxypropyltriazolylmethyl)-amine (THPTA, 0.68 mL of 10.0 mM, 20 eq), and sodium ascorbate (1.0 mL of 10.0 mM, 30 eq). The resulting solution was agitated gently for 12 hours at room temperature. The reaction mixture was purified by affinity chromatography over a protein A column, followed size exclusion chromatography (See Example 164). MALDI TOF analysis of the purified final product gave an average mass of 69,330 Da (DAR=3.3). Yield 10 mg, 50% yield.

### Example 123—Synthesis of Conjugate 31

A solution of azido-hIgG1(Myc)Fc in PBS buffer (30 mg, 0.50 μmol, 2.0 mL, MW=58,743 Da, DAR=3.3) was treated with alkyne derivatized small molecule (TFA salt, 54 mg, 13.5 μmol, Example 71) and freshly prepared pH 7.4 PBS solutions of CuSO₄ (1.0 mL of 10.0 mM, 20 eq), tris(3-hydroxypropyltriazolylmethyl)-amine (THPTA, 1.0 mL of 10.0 mM, 20 eq), and sodium ascorbate (1.4 mL of 10.0 mM, 30 eq). The resulting solution was agitated gently for 12 hours at room temperature. The reaction mixture was purified by affinity chromatography over a protein A column, followed size exclusion chromatography (See Example 164). MALDI TOF analysis of the purified final product gave an average mass of 64462 Da (DAR=2.17). Yield 26.1 mg, 65% yield.

### Example 124—Synthesis of Conjugate 33

A solution of azido-hIgG1(Myc)Fc in PBS buffer (30 mg, 0.50 μmol, 2.0 mL, MW=58,548 Da, DAR=2.7) was treated with alkyne derivatized small molecule (TFA salt, 54 mg, 13.5 μmol, Example 72) and freshly prepared pH 7.4 PBS solutions of CuSO₄ (1.0 mL of 10.0 mM, 20 eq), tris(3-hydroxypropyltriazolylmethyl)-amine (THPTA, 1.0 mL of 10.0 mM, 20 eq), and sodium ascorbate (1.4 mL of 10.0 mM, 30 eq). The resulting solution was agitated gently for 12 hours at room temperature. The reaction mixture was purified by affinity chromatography over a protein A column, followed size exclusion chromatography (See Example 164). MALDI TOF analysis of the purified final product gave an average mass of 64.223 Da (DAR=2.05). Yield 14.5 mg, 48% yield.

### Example 125—Synthesis of Conjugate 44

A solution of azido-hIgG1(Myc)Fc in PBS buffer (30 mg, 0.50 μmol, 2.0 mL. MW=58,935 Da, DAR=3.88) was treated with alkyne derivatized small molecule (TFA salt, 58 mg, 15 μmol. Example 73) and freshly prepared pH 7.4 PBS solutions of CuSO₄ (1.0 mL of 10.0 mM, 20 eq), tris(3-hydroxypropyltriazolylmethyl)-amine (THPTA, 1.0 mL of 10.0 mM, 20 eq), and sodium ascorbate (1.5 mL of 10.0 mM, 30 eq). The resulting solution was agitated gently for 12 hours at room temperature. The reaction mixture was purified by affinity chromatography over a protein A column, followed size exclusion chromatography (See Example 164). MALDI TOF analysis of the purified final product gave an average mass of 69,725 Da (DAR=4). Yield 27 mg, 91% yield.

### Example 126—Synthesis of Conjugate 66

A solution of azido-hIgG1(Myc)Fc in PBS buffer (20 mg, 0.34 μmol, 2.0 mL, MW=58,721 Da, DAR=2.75) was treated with alkyne derivatized small molecule (TFA salt, 35 mg, 9.2 μmol, Example 74) and freshly prepared pH 7.4 PBS solutions of CuSO₄ (0.7 mL of 10.0 mM, 20 eq), tris(3-hydroxypropyltriazolylmethyl)-amine (THPTA, 0.7 mL of 10.0 mM, 20 eq), and sodium ascorbate (1.0 mL of 10.0 mM, 30 eq). The resulting solution was agitated gently for 12 hours at room temperature. The reaction mixture was purified by affinity chromatography over a protein A column, followed size exclusion chromatography (See Example 164). MALDI TOF analysis of the purified final product gave an average mass of 64,614 Da (DAR=2.3). Yield 15.3 mg, 77% yield.

### Example 127—Synthesis of Conjugate 67

A solution of azido-hIgG1(Myc)Fc in PBS buffer (20 mg, 0.34 μmol, 2.0 mL, MW=58,721 Da, DAR=2.75) was treated with alkyne derivatized small molecule (TFA salt, 34 mg, 9.2 μmol, Example 75) and freshly prepared pH 7.4 PBS solutions of CuSO₄ (0.7 mL of 10.0 mM, 20 eq), tris(3-hydroxypropyltriazolylmethyl)-amine (THPTA, 0.7 mL of 10.0 mM, 20 eq), and sodium ascorbate (1.0 mL of 10.0 mM, 30 eq). The resulting solution was agitated gently for 12 hours at room temperature. The reaction mixture was purified by affinity chromatography over a protein A column, followed size exclusion chromatography (See Example 164). MALDI TOF analysis of the purified final product gave an average mass of 64,792 Da (DAR=2.4). Yield 14.4 mg, 72% yield.

### Example 128—Synthesis of Conjugate 68

A solution of azido-hIgG1(Myc)Fc in PBS buffer (20 mg, 0.34 μmol, 1.40 mL, MW=58,721 Da, DAR=2.7) was treated with alkyne derivatized small molecule (TFA salt, 36 mg, 9.2 μmol, Example 76) and freshly prepared pH 7.4 PBS solutions of CuSO₄ (0.7 mL of 10.0 mM, 20 eq), tris(3-hydroxypropyltriazolylmethyl)-amine (THPTA, 0.7 mL of 10.0 mM, 20 eq), and sodium ascorbate (1.02 mL of 10.0 mM, 30 eq). The resulting solution was agitated gently for 12 hours at room temperature. The reaction mixture was purified by affinity chromatography over a protein A column, followed size exclusion chromatography (See Example 164). MALDI TOF analysis of the purified final product gave an average mass of 65.317 Da (DAR=2.4). Yield 13.4 mg, 67% yield.

### Example 129—Synthesis of Conjugate 56

A solution of azido-hIgG1(Myc)Fc in PBS buffer (20 mg, 0.34 μmol, 1.40 mL, MW=58526 Da, DAR=2.59) was treated with alkyne derivatized small molecule (TFA salt, 36 mg, 9.2 μmol, Example 77) and freshly prepared pH 7.4 PBS solutions of CuSO₄ (0.7 mL of 10.0 mM, 20 eq), tris(3-hydroxypropyltriazolylmethyl)-amine (THPTA, 0.7 mL of 10.0 mM, 20 eq), and sodium ascorbate (1.02 mL of 10.0 mM, 30 eq). The resulting solution was agitated gently for 12 hours at room temperature. The reaction mixture was purified by affinity chromatography over a protein A column, followed size exclusion chromatography (See Example 164). MALDI TOF analysis of the purified final product gave an average mass of 63,622 Da (DAR=2.21). Yield 19.2 mg, 96% yield.

### Example 130—Synthesis of Conjugate 79

A solution of azido-hIgG1(Myc)Fc in PBS buffer (20 mg, 0.34 μmol, 1.40 mL, MW=58833 Da, DAR=3.16) was treated with alkyne derivatized small molecule (TFA salt, 43 mg, 10.2 μmol, Example 78) and freshly prepared pH 7.4 PBS solutions of CuSO₄ (0.7 mL of 10.0 mM, 20 eq), tris(3-hydroxypropyltriazolylmethyl)-amine (THPTA, 0.7 mL of 10.0 mM, 20 eq), and sodium ascorbate (1.02 mL of 10.0 mM, 30 eq). The resulting solution was agitated gently for 12 hours at room temperature. The reaction mixture was purified by affinity chromatography over a protein A column, followed size exclusion chromatography (See Example 164). MALDI TOF analysis of the purified final product gave an average mass of 65,326 Da (DAR=2.1). Yield 11.8 mg, 59% yield.

### Example 131—Synthesis of Conjugate 32

A solution of azido-hIgG1(Myc)Fc in PBS buffer (40 mg, 0.67 μmol, 2.50 mL, MW=58743 Da, DAR=3.3) was treated with alkyne derivatized small molecule (TFA salt, 46 mg, 9.2 μmol, Example 79) and freshly prepared pH 7.4 PBS solutions of CuSO₄ (1.4 mL of 10.0 mM, 20 eq), tris(3-hydroxypropyltriazolylmethyl)-amine (THPTA, 1.4 mL of 10.0 mM, 20 eq), and sodium ascorbate (1.4 mL of 10.0 mM, 30 eq). The resulting solution was agitated gently for 12 hours at room temperature. The reaction mixture was purified by affinity chromatography over a protein A column, followed size exclusion chromatography (See Example 164). MALDI TOF analysis of the purified final product gave an average mass of 62,465 Da (DAR=2.65). Yield 13.8 mg, 35% yield.

### Example 132—Synthesis of Conjugate 46

A solution of azido-hIgG1(Myc)Fc in PBS buffer (22.5 mg, 0.38 μmol, 1.50 mL, MW=58,835 Da, DAR=5) was treated with alkyne derivatized small molecule (TFA salt, 23 mg, 11 μmol, Example 80) and freshly prepared pH 7.4 PBS solutions of CuSO₄ (0.75 mL of 10.0 mM, 20 eq), tris(3-hydroxypropyltriazolylmethyl)-amine (THPTA, 0.75 mL of 10.0 mM, 20 eq), and sodium ascorbate (1.13 mL of 10.0 mM, 30 eq). The resulting solution was agitated gently for 12 hours at room temperature. The reaction mixture was purified by affinity chromatography over a protein A column, followed size exclusion chromatography (See Example 164). MALDI TOF analysis of the purified final product gave an average mass of 65.241 Da (DAR=4.18). Yield 15 mg. 75% yield.

### Example 133—Synthesis of Conjugate 46

A solution of azido-hIgG1(Myc)Fc in PBS buffer (20 mg, 0.32 μmol, 2.50 mL, MW=59641 Da, DAR=3.9) was treated with alkyne derivatized small molecule (TFA salt, 19 mg, 10 μmol, Example 81) and freshly prepared pH 7.4 PBS solutions of CuSO₄ (0.65 mL of 10.0 mM, 20 eq), tris(3-hydroxypropyltriazolylmethyl)-amine (THPTA, 0.65 mL of 10.0 mM, 20 eq), and sodium ascorbate (1.0 mL of 10.0 mM, 30 eq). The resulting solution was agitated gently for 12 hours at room temperature. The reaction mixture was purified by affinity chromatography over a protein A column, followed size exclusion chromatography (See Example 164). MALDI TOF analysis of the purified final product gave an average mass of 64,789 Da (DAR=4.26). Yield 17.88 mg, 89% yield.

### Example 134—Synthesis of Conjugate 42

A solution was freshly prepared with CuSO₄ (10 mM, 16.07 mg), sodium ascorbate (20 mM, 39.7 mg) and tris(3-hydroxypropyltriazolylmethyl)amine) (10 mM, 43.86 mg) in PBS 7.4 buffer solution of (10 mL).

A solution of azido-hIgG1(Myc)Fc (40 mg, 0.679 μmol, 15.0 mg/mL, MW=58,935 Da, DAR=5.0) in pH 7.4 PBS was treated with freshly prepared PBS 7.4 solutions (1.68 mL) of alkyne derivatized small molecule TFA salt (97.0 mg, 27.1 μmol Example 84). To this solution was added a freshly prepared mixed solution of CuSO₄ [(10 mM), sodium ascorbate (20 mM) and tris(3-hydroxypropyltriazolylmethyl)amine) (10 mM), 1.4 mL]. The resulting solution was gently agitated on a rocker table at room temperature for 3 hours then another 0.5 mL of the mixed solution [CuSO₄ (10 mM), sodium ascorbate (20 mM) and tris(3-hydroxypropyltriazolylmethyl)amine) (10 mM)]. The resulting solution was continued to agitate overnight then purified by affinity chromatography over a protein-A column. See Example 164 for purification procedure. Maldi TOF analysis of the purified final product gave an average mass of 69,638 Da (DAR=4.0). Yield 37.53 mg, 79% yield.

### Example 135—Synthesis of Conjugate 53

A solution was freshly prepared with CuSO₄ (10 mM, 15.96 mg), sodium ascorbate (20 mM, 39.6 mg) and tris(3-hydroxypropyltriazolylmethyl)amine) (10 mM, 43.86 mg) in PBS 7.4 buffer solution of (10 mL).

A solution of azido-hIgG1(Myc)Fc (20 mg, 1.43 mL, 0.342 μmol, 14.0 mg/mL, MW=58,526 Da, DAR=2.6) in pH 7.4 PBS was diluted with PBS 7.4 solutions (0.57 mL) to make a 10.0 mg/mL solution of (2 mL).

A solution of alkyne derivatized small molecule TFA salt (27.5 mg, 7.08 μmol, Example 88) in PBS 7.4 solutions (1.5 mL) was prepared in a 15 mL centrifuge tube. To this solution was added A solution of (0.68 mL, 10 mM of CuSO₄, 20 mM of sodium ascorbate and 10 mM tris(3-hydroxypropyltriazolylmethyl)amine)) followed by addition of a solution of azido-hIgG1(Myc)Fc (20 mg, 2.0 mL, 0.342 μmol, 10.0 mg/mL, MW=58,526 Da, DAR=2.59) in pH 7.4 PBS. The resulting solution was gently agitated on a rocker table at room temperature overnight then purified by affinity chromatography over a protein-A column. See Example 164 for purification procedure. Maldi TOF analysis of the purified final product gave an average mass of 65.320 Da (DAR=2.5). Yield 19.15 mg, 87% yield.

### Example 136—Synthesis of Conjugate 54

A solution was freshly prepared with CuSO₄ (10 mM, 15.96 mg), sodium ascorbate (20 mM, 39.6 mg) and tris(3-hydroxypropyltriazolylmethyl)amine) (10 mM, 43.86 mg) in PBS 7.4 buffer solution of (10 mL).

A solution of azido-hIgG1(Myc)Fc (20 mg, 1.43 mL, 0.342 μmol, 14.0 mg/mL, MW=58,526 Da, DAR=2.59) in pH 7.4 PBS was diluted with PBS 7.4 solutions (0.57 mL) to make a 10.0 mg/mL solution of (2 m).

A solution of alkyne derivatized small molecule TFA salt (25.7 mg, 7.08 μmol, Example 87) in PBS 7.4 solutions (1.5 mL) was prepared in a 15 mL centrifuge tube. To this solution was added A solution of (0.68 mL, 10 mM of CuSO₄, 20 mM of sodium ascorbate and 10 mM tris(3-hydroxypropyltriazolylmethyl)amine)) followed by addition of a solution of azido-hIgG1(Myc)Fc (20 mg, 2.0 mL, 0.342 μmol, 10.0 mg/mL, MW=58,526 Da, DAR=2.59) in pH 7.4 PBS. The resulting solution was gently agitated on a rocker table at room temperature overnight then purified by affinity chromatography over a protein-A column. See Example 164 for purification procedure. Maldi TOF analysis of the purified final product gave an average mass of 64,708 Da (DAR=2.3). Yield 19.32 mg, 88% yield.

### Example 137—Synthesis of Conjugate 62

A solution was freshly prepared with CuSO₄ (10 mM, 15.96 mg), sodium ascorbate (20 mM, 39.6 mg) and tris(3-hydroxypropyltriazolylmethyl)amine) (10 mM, 43.86 mg) in PBS 7.4 buffer solution of (10 mL).

A solution of azido-hIgG1(Myc)Fc (20 mg, 1.43 mL, 0.342 μmol, 14.0 mg/mL, MW=58,721 Da, DAR=2.75) in pH 7.4 PBS was diluted with PBS 7.4 solutions (0.57 mL) to make a 10.0 mg/mL solution of (2 mL).

A solution of alkyne derivatized small molecule TFA salt (36.1 mg, 9.36 μmol, Example 89) in PBS 7.4 solutions (1.5 mL) was prepared in a 15 mL centrifuge tube. To this solution was added A solution of (0.68 mL, 10 mM of CuSO₄, 20 mM of sodium ascorbate and 10 mM tris(3-hydroxypropyltriazolylmethyl)amine)) followed by addition of a solution of azido-hIgG1(Myc)Fc (20 mg, 2.0 mL, 0.342 μmol, 10.0 mg/mL, MW=58,721 Da, DAR=2.75) in pH 7.4 PBS. The resulting solution was gently agitated on a rocker table at room temperature overnight then purified by affinity chromatography over a Superdex200 column. See Example 164 for purification procedure. Maldi TOF analysis of the purified final product gave an average mass of 66,835 Da (DAR=3.0). Yield 9.86 mg, 41% yield.

### Example 138—Synthesis of Conjugate 63

A solution was freshly prepared with CuSO₄ (10 mM, 15.96 mg), sodium ascorbate (20 mM, 39.6 mg) and tris(3-hydroxypropyltriazolylmethyl)amine) (10 mM, 43.86 mg) in PBS 7.4 buffer solution of (10 mL).

A solution of azido-hIgG1(Myc)Fc (20 mg, 1.43 mL, 0.342 μmol, 14.0 mg/mL. MW=58.721 Da, DAR=2.75) in pH 7.4 PBS was diluted with PBS 7.4 solutions (0.57 mL) to make a 10.0 mg/mL solution of (2 mL).

A solution of alkyne derivatized small molecule TFA salt (32.8 mg, 9.2 μmol, Example 86) in PBS 7.4 solutions (1.5 mL) was prepared in a 15 mL centrifuge tube. To this solution was added A solution of (0.68 mL, 10 mM of CuSO₄, 20 mM of sodium ascorbate and 10 mM tris(3-hydroxypropyltriazolylmethyl)amine)) followed by addition of a solution of azido-hIgG1(Myc)Fc (20 mg, 2.0 mL, 0.342 μmol, 10.0 mg/mL, MW=58,721 Da, DAR=2.75) in pH 7.4 PBS. The resulting solution was gently agitated on a rocker table at room temperature overnight then purified by protein-A affinity chromatography. See Example 164 for purification procedure. Maldi TOF analysis of the purified final product gave an average mass of 66,609 Da (DAR=2.9). Yield 9.55 mg, 41% yield.

### Example 139—Synthesis of Conjugate 64

A solution was freshly prepared with CuSO₄ (10 mM, 15.96 mg), sodium ascorbate (20 mM, 39.6 mg) and tris(3-hydroxypropyltriazolylmethyl)amine) (10 mM, 43.86 mg) in PBS 7.4 buffer solution of (10 mL).

A solution of azido-hIgG1(Myc)Fc (20 mg, 1.43 mL, 0.342 μmol, 14.0 mg/mL, MW=58,721 Da, DAR=2.75) in pH 7.4 PBS was diluted with PBS 7.4 solutions (0.57 mL) to make a 10.0 mg/mL solution of (2 mL).

A solution of alkyne derivatized small molecule TFA salt (35.9 mg, 9.37 μmol, Example 85) in PBS 7.4 solutions (1.5 mL) was prepared in a 15 mL centrifuge tube. To this solution was added A solution of (0.68 mL, 10 mM of CuSO₄, 20 mM of sodium ascorbate and 10 mM tris(3-hydroxypropyltriazolylmethyl)amine)) followed by addition of a solution of azido-hIgG1(Myc)Fc (20 mg, 2.0 mL, 0.342 μmol, 10.0 mg/mL, MW=58,721 Da, DAR=2.75) in pH 7.4 PBS. The resulting solution was gently agitated on a rocker table at room temperature overnight then purified by affinity chromatography over a Superdex200 column. See Example 164 for purification procedure. Maldi TOF analysis of the purified final product gave an average mass of 66,863 Da (DAR=3.0). Yield 8.62 mg, 36% yield.

### Example 140—Synthesis of Conjugate 65

A solution was freshly prepared with CuSO₄ (10 mM, 15.96 mg), sodium ascorbate (20 mM, 39.6 mg) and tris(3-hydroxypropyltriazolylmethyl)amine) (10 mM, 43.86 mg) in PBS 7.4 buffer solution of (10 mL).

A solution of azido-hIgG1(Myc)Fc (20 mg, 1.43 mL, 0.342 μmol, 14.0 mg/mL. MW=58.721 Da, DAR=2.75) in pH 7.4 PBS was diluted with PBS 7.4 solutions (0.57 mL) to make a 10.0 mg/mL solution of (2 mL).

A solution of alkyne derivatized small molecule TFA salt (36.4 mg, 9.37 μmol, Example 82) in PBS 7.4 solutions (1.5 mL) was prepared in a 15 mL centrifuge tube. To this solution was added A solution of (0.68 mL, 10 mM of CuSO₄, 20 mM of sodium ascorbate and 10 mM tris(3-hydroxypropyltriazolylmethyl)amine)) followed by addition of a solution of azido-hIgG1(Myc)Fc (20 mg, 2.0 mL, 0.342 μmol, 10.0 mg/mL, MW=58,721 Da, DAR=2.75) in pH 7.4 PBS. The resulting solution was gently agitated on a rocker table at room temperature overnight then purified by protein-A affinity chromatography. See Example 164 for purification procedure. Maldi TOF analysis of the purified final product gave an average mass of 65,998 Da (DAR=2.7). Yield 10.48 mg, 46% yield.

### Example 141—Synthesis of Conjugate 73

A solution was freshly prepared with CuSO₄ (10 mM, 8.2 mg), sodium ascorbate (20 mM, 22.27 mg) and tris(3-hydroxypropyltriazolylmethyl)amine) (10 mM, 22.0 mg) in PBS 7.4 buffer solution of (5 mL).

A solution of azido-hIgG1(Myc)Fc (20 mg, 1.43 mL, 0.342 μmol, 14.0 mg/mL, MW=58,721 Da, DAR=2.75) in pH 7.4 PBS was diluted with PBS 7.4 solutions (0.57 mL) to make a 10.0 mg/mL solution of (2 mL).

A solution of alkyne derivatized small molecule TFA salt (36.96 mg, 9.37 μmol, Example 83) in PBS 7.4 solutions (1.5 mL) was prepared in a 15 mL centrifuge tube. To this solution was added A solution of (0.68 mL, 10 mM of CuSO₄, 20 mM of sodium ascorbate and 10 mM tris(3-hydroxypropyltriazolylmethyl)amine)) followed by addition of a solution of azido-hIgG1(Myc)Fc (20 mg, 2.0 mL, 0.342 μmol, 10.0 mg/mL, MW=58,721 Da, DAR=2.75) in pH 7.4 PBS. The resulting solution was gently agitated on a rocker table at room temperature overnight then purified by affinity chromatography over a protein-A column. See Example 164 for purification procedure. Maldi TOF analysis of the purified final product gave an average mass of 68,785 Da (DAR=3.5). Yield 16.64 mg, 58% yield.

### Example 142—Synthesis of Conjugate 35

Alkyne derivatized small molecule TFA salt (53 mg, 0.13 mmol, See Example 95) was dissolved in 1 mL of pH 7.4 PBS buffer and added to azido-hIgG1(Myc)Fc (30 mg, 51 μmol, MW=58548, DAR 2.7) in 4 mL of PBS 7.4 buffer followed by (1.02 mL) of a premixed solution which was 20 mM sodium ascorbate (0.02 mmol), 10 mM Copper (II) sulfate (0.01 mmol) and 10 mM tris(3-hydroxypropyltriazolylmethyl)amine (0.01 mmol) and the reaction was shaken gently at ambient temperature for 16 hours. See Example 164 for purification procedure. Yield of 19.9 mg, 60%. MALDI MS analysis showed a range of masses (58264-72608) with an average of mass of 64013. Average DAR=2.4.

### Example 143—Synthesis of Conjugate 36

Alkyne derivatized small molecule TFA salt (49 mg, 0.13 mmol, See Example 93) was dissolved in 1 mL of pH 7.4 PBS buffer and added to azido-hIgG1(Myc)Fc (30 mg, 51 μmol, MW=58548, DAR 2.7) in 4 mL of PBS 7.4 buffer followed by (1.02 mL) of a premixed solution which was 20 mM sodium ascorbate (0.02 mmol), 10 mM Copper (II) sulfate (0.01 mmol) and 10 mM tris(3-hydroxypropyltriazolylmethyl)amine (0.01 mmol) and the reaction was shaken gently at ambient temperature for 16 hours. See Example 164 for purification procedure. Yield of 18.9 mg, 61%. MALDI MS analysis showed a range of masses (58270-72456) with an average of mass of 63816. Average DAR=2.3.

### Example 144—Synthesis of Conjugate 37

Alkyne derivatized small molecule TFA salt (49 mg, 0.13 mmol, See Example 94) was dissolved in 1 mL of pH 7.4 PBS buffer and added to azido-hIgG1(Myc)Fc (30 mg, 51 μmol, MW=58548, DAR 2.7) in 4 mL PBS 7.4 buffer followed by (1.02 mL) of a premixed solution which was 20 mM sodium ascorbate (0.02 mmol), 10 mM Copper (II) sulfate (0.01 mmol) and 10 mM tris(3-hydroxypropyltriazolylmethyl)amine (0.01 mmol) and the reaction was shaken gently at ambient temperature for 16 hours. See Example 164 for purification procedure. Yield of 23.4 mg, 71%. MALDI MS analysis showed a range of masses (58273-72500) with an average of mass of 63921. Average DAR=2.1.

### Example 145—Synthesis of Conjugate 49

Alkyne derivatized small molecule TFA salt (53 mg, 0.0085 mmol, Example 90) was dissolved in 1 mL of pH 7.4 PBS buffer and added to azido-hIgG1(Myc)Fc (20 mg, 34 μmol, MW=58526, DAR 2.6) in 3 mL PBS 7.4 buffer followed by (0.68 mL) of a premixed solution which was 20 mM sodium ascorbate (0.014 mmol), 10 mM Copper (II) sulfate (0.0068 mmol) and 10 mM tris(3-hydroxypropyltriazolylmethyl)amine (0.0068 mmol) and the reaction was shaken gently at ambient temperature for 16 hours. See Example 164 for purification procedure. Yield of 17.9 mg, 81%. MALDI MS analysis showed a range of masses (58344-72906) with an average of mass of 64071. Average DAR=2.4.

### Example 146—Synthesis of Conjugate 50

Alkyne derivatized small molecule TFA salt (31 mg, 0.0085 mmol, See Example 97) was dissolved in 1 mL of pH 7.4 PBS buffer and added to azido-hIgG1(Myc)Fc (20 mg, 34 μmol, MW=58526, DAR 2.6) in 3 mL PBS 7.4 buffer followed by (0.68 mL) of a premixed solution which was 20 mM sodium ascorbate (0.014 mmol), 10 mM Copper (II) sulfate (0.0068 mmol) and 10 mM tris(3-hydroxypropyltriazolylmethyl)amine (0.0068 mmol) and the reaction was shaken gently at ambient temperature for 16 hours. See Example 164 for purification procedure. Yield of 20.6 mg, 94%. MALDI MS analysis showed a range of masses (58346-72992) with an average of mass of 64001. Average DAR=2.5.

### Example 147—Synthesis of Conjugate 51

Alkyne derivatized small molecule TFA salt (33 mg, 0.0085 mmol, See Example 96) was dissolved in 1 mL of pH 7.4 PBS buffer and added to azido-hIgG1(Myc)Fc (20 mg, 34 μmol, MW=58526, DAR 2.6) in 3 mL PBS 7.4 buffer followed by (0.68 mL) of a premixed solution which was 20 mM sodium ascorbate (0.014 mmol), 10 mM Copper (II) sulfate (0.0068 mmol) and 10 mM tris(3-hydroxypropyltriazolylmethyl)amine (0.0068 mmol) and the reaction was shaken gently at ambient temperature for 16 hours. See Example 164 for purification procedure. Yield of 19.3 mg, 88%. MALDI MS analysis showed a range of masses (58289-72667) with an average of mass of 64060. Average DAR=2.6.

### Example 148—Synthesis of Conjugate 57

Alkyne derivatized small molecule TFA salt (33 mg, 0.0085 mmol, See Example 91) was dissolved in 1 mL of pH 7.4 PBS buffer and added to azido-hIgG1(Myc)Fc (20 mg, 34 μmol, MW=58526, DAR 2.6) in 3 mL PBS 7.4 buffer followed by (0.68 mL) of a premixed solution which was 20 mM sodium ascorbate (0.014 mmol), 10 mM Copper (II) sulfate (0.0068 mmol) and 10 mM tris(3-hydroxypropyltriazolylmethyl)amine (0.0068 mmol) and the reaction was shaken gently at ambient temperature for 16 hours. See Example 164 for purification procedure. Yield of 9.96 mg, 44%. MALDI MS analysis showed a range of masses (58980-76291) with an average of mass of 64526. Average DAR=3.0.

### Example 149—Synthesis of Conjugate 61

Alkyne derivatized small molecule TFA salt (32 mg, 0.0092 mmol, See Example 98) was dissolved in 1 mL of pH 7.4 PBS buffer and added to azido-hIgG1(Myc)Fc (20 mg, 34 μmol, MW=58721, DAR 2.8) in 3 mL PBS 7.4 buffer followed by (0.68 mL) of a premixed solution which was 20 mM sodium ascorbate (0.014 mmol), 10 mM Copper (II) sulfate (0.0068 mmol) and 10 mM tris(3-hydroxypropyltriazolylmethyl)amine (0.0068 mmol) and the reaction was shaken gently at ambient temperature for 16 hours. See Example 164 for purification procedure. Yield of 9.86 mg, 42%. MALDI MS analysis showed a range of masses (58258-75079) with an average of mass of 66516. Average DAR=2.9.

### Example 150—Synthesis of Conjugate 72

Alkyne derivatized small molecule TFA salt (34 mg, 0.0092 mmol, See Example 99) was dissolved in 1 mL of pH 7.4 PBS buffer and added to azido-hIgG1(Myc)Fc (20 mg, 34 μmol, MW=58721, DAR 2.8) in 3 mL PBS 7.4 buffer followed by (0.68 mL) of a premixed solution which was 20 mM sodium ascorbate (0.014 mmol), 10 mM Copper (II) sulfate (0.0068 mmol) and 10 mM tris(3-hydroxypropyltriazolylmethyl)amine (0.0068 mmol) and the reaction was shaken gently at ambient temperature for 16 hours. See Example 164 for purification procedure. Yield of 14.7 mg, 63%. MALDI MS analysis showed a range of masses (58687-76383) with an average of mass of 67633. Average DAR=3.4.

### Example 151—Synthesis of Conjugate 77

Alkyne derivatized small molecule TFA salt (34 mg, 0.0092 mmol, See Example 92) was dissolved in 1 mL of pH 7.4 PBS buffer and added to azido-hIgG1(Myc)Fc (20 mg, 34 μmol, MW=58721, DAR 2.8) in 3 mL PBS 7.4 buffer followed by (0.68 mL) of a premixed solution which was 20 mM sodium ascorbate (0.015 mmol), 10 mM Copper (II) sulfate (0.0076 mmol) and 10 mM tris(3-hydroxypropyltriazolylmethyl)amine (0.076 mmol) and the reaction was shaken gently at ambient temperature for 16 hours. See Example 164 for purification procedure. Yield of 16.42 mg, 63%. MALDI MS analysis showed a range of masses (58521-76178) with an average of mass of 68384. Average DAR=3.1.

### Example 152—Synthesis of Conjugate 24

Note: all the solutions were prepared in PBS 7.4 buffer solution. Product described in Example 100 (60 mg, 0.0169 mmol) was added to azido-hIgG1(Myc)Fc (25 mg, 0.42 μmol, 12.4 mg/mL, MW=59,641 Da, DAR=5) solution, followed by 0.832 mL of 0.01 M CuSO₄ solution, 0.832 mL of 0.01 M sodium ascorbate solution, and 0.832 mL of 0.01 M THPTA solution. The reaction was gently shaken at room temperature for 4 hours, then treated with an additional amount of freshly prepared CuSO₄ solution (0.01 M, 0.416 mL). The reaction was shaken overnight. Additional CuSO₄ solution (0.01 M, 0.416 mL, freshly prepared) was added, and the reaction mixture was shaken for another 2 hours. The resulting solution was purified by affinity chromatography over a protein A column, followed size exclusion chromatography (See Example 164). Maldi TOF analysis of the purified final product gave an average mass of 71,189. Da (DAR=4.2). Yield 24.5 mg, 99% yield.

### Example 153—Synthesis of Conjugate 48

Note: All the solutions were prepared in PBS 7.4 buffer solution. The material described in Example 101(16 mg, 0.004043 mmol) was added to azido-hIgG1(Myc)Fc (30 mg, 0.51 μmol, 13.6 mg/mL, MW=59367 Da, DAR=4), followed by 1.263 mL of 0.01 M CuSO₄, 2.021 mL of 0.01 M sodium ascorbate, and 1.263 mL of 0.01 M THPTA solutions. The reaction was gently shaken at room temperature overnight. It was then treated with an additional amount of alkyne (48 mg, 0.01213 mmol), followed by 1.263 mL of 0.01 M CuSO₄, 2.021 mL of 0.01 M sodium ascorbate, and 1.263 mL of 0.01 M THPTA solutions. The reaction was shaken for 1 more day. The resulting solution was purified by affinity chromatography over a protein A column, followed size exclusion chromatography (See Example 164). Maldi TOF analysis of the purified final product gave an average mass of 68611. Da (DAR=3.6). Yield 28.56 mg, 95% yield.

### Example 154—Synthesis of Conjugate 58

Note: All the solutions were prepared in PBS 7.4 buffer solution. To a solution of azido-hIgG1(Myc)Fc (26 mg, 0.44 μmol, 14 mg/mL, MW=58526 Da, DAR=2.59) was added product from Example 102 (37.1 mg, 0.009205 mmol, 0.5 mL). After the resulting mixture was gently mixed, it was treated with 1.1106 mL of 0.01 M CuSO₄, 1.777 mL of 0.01 M sodium ascorbate, and 1.1106 mL of 0.01 M THPTA solutions. The reaction was gently shaken at room temperature overnight. It was then purified by affinity chromatography over a protein A column, followed size exclusion chromatography (See Example 164). Maldi TOF analysis of the purified final product gave an average mass of 66062. Da (DAR=2.7). Yield 23.46 mg, 90% yield

### Example 155—Synthesis of Conjugate 59

The title compound was prepared analogously to Example 155 substituting product from Example 103. Maldi TOF analysis of the purified final product gave an average mass of 66111. Da (DAR=2.7). Yield 22.72 mg, 84% yield.

### Example 156—Synthesis of Conjugate 60

The title compound was prepared analogously to Example 155 using product from Example 104. Maldi TOF analysis of the purified final product gave an average mass of 65,962. Da (DAR=2.6). Yield 16.37 mg, 82% yield

### Example 157—Synthesis of Conjugate 70

Note: all the solutions were prepared in PBS 7.4 buffer solution. To a solution of product from Example 105 (30 mg, 0.007518 mmol, 0.5 mL) was added azido-hIgG1(Myc)Fc (20 mg, 0.34 μmol, 14.3 mg/mL, MW=58721 Da, DAR=2.75). The resulting mixture was gently mixed and then treated with 0.3 mL of 0.05 M CuSO₄ and 0.6 mL of 0.05 M sodium ascorbate solutions. After gently mixed, the reaction mixture was treated with 0.3 mL of 0.05 M THPTA solution and gently shaken at room temperature for 4 hours. It was then treated with an additional amount CuSO₄ (2 mg, 0.0125 mmol), and the reaction was continued shaken overnight. The resulting solution was purified by affinity chromatography over a protein A column, followed size exclusion chromatography (See Example 164). Maldi TOF analysis of the purified final product gave an average mass of 65391. Da (DAR=2.5). Yield 5.27 mg, 39% yield.

### Example 158—Synthesis of Conjugate 71

The title compound was prepared analogously to Example 158 using the product from Example 106. Maldi TOF analysis of the purified final product gave an average mass of 64,733. Da (DAR=2.2). Yield 4.07 mg, 42% yield.

### Example 159—Synthesis of Azido-hIgG1(Myc)Fc with Specific DAR Values

Preparation of solutions of azido-PEG4 NHS ester in DMF/PBS solution: 100.0 mg of Azido-PEG4 NHS ester was dissolved in 1.00 mL of DMF and diluted with 2.57 mL of PBS buffer solution in ice bath to give 0.1 M azido-PEG4 NHS ester solution. 1.00 mL of 0.1 M azido-PEG4 NHS ester was diluted to 0.050M PBS buffer solution. A solution of hIgG1(Myc)Fc (described in Example 1) in pH 7.4 PBS solution (2.31 mL, 5.17 μmol, 13.0 mg/mL, MW=58009 Da) was treated with azido-PEG4-NHS ester as shown in the table below, then agitated gently overnight at RT to give azido-hIgG1(Myc)Fc with specific DAR values. Unreacted azido-PEG4-NHS ester was removed by buffer exchange with a 10,000 MWCO centrifugal filter (6×15 mL pH 7.4 PBS). The final average molecular weight of the azido functionalized Fc was measured by MALDI-TOF (Table 7 and FIG. 103). The DAR values were calculated with the following formula: DAR (MW of azido-Fc—MW of Fc)+(MW azido PEG4 fragment=273.3). Using 0 to 20 equivalents of azido PEG4 NHS ester, gives the corresponding azido-DAR values.

TABLE 7

| | | | | Azido PEG4 | | | | | |
|---|---|---|---|---|---|---|---|---|---|
| Equivalents | | | | NHS ester | | | | | |
| of Azido | hlgG1 (Myc) Fc | | | Solution | | Azido-Fc | | | |
| PEG4-NHS | Batch | MW (Da) | | (μL) | | MALDI | | ESI | |
| ester | Name | MALDI | ESI | 0.1M | 0.05M | MW (Da) | DAR | MW (Da) | DAR |
| 5 | Azido | 58009 | 58275 | | 52 | 58350 | 1.3 | 58825 | 2 |
| 10 | Fc | | | | 103 | 58947 | 3.4 | 59371 | 4 |
| 20 | | | | 103 | | 60020 | 7.4 | 60465 | 8 |
| 40 | | | | 207 | | 60944 | 10.7 | 61831 | 13 |
| 100 | | | | 517 | | 61868 | 14.1 | | |

### Example 160—Synthesis of Conjugate 30

A solution of hIgG1(Myc)Fc (22.00 mL, 4.68 μmol, 12.3 mg/mL. MW=57812 Da) in pH 7.4 PBS was treated with azido-PEG4-NHS ester (0.0107 g, 562 μL of 0.050 M solution in DMF/PBS (1:3). 281□μmol, 6 eq), then agitated gently overnight at RT. Unreacted azido-PEG4-NHS ester was removed by buffer exchange with a 10,000 MWCO centrifugal filter (6×15 mL pH 7.4 PBS). The final volume of the azide functionalized Fc (143-4A-azido-Fc) was 19 mL with concentration at 14.7 mg/mL, MW (MALDI)=58548. DAR (MALDI)=2.7. To this azido-Fc PBS buffer solution (0.0294 g, 0.502 μmol, 2.00 mL) were added alkyne derivatized small molecule (TFA salt, 52.3 mg, 13.56□μmol, Example 107) and freshly prepared pH 7.4 PBS solutions of CuSO₄ (1.004 mL of 10.0 mM, 20 eq), tris(3-hydroxypropyltriazolylmethyl)-amine (1.004 mL of 10.0 mM, 20 eq), and sodium ascorbate (1.004 mL of 10.0 mM, 20 eq). The resulting solution was agitated gently for 4 hours at room temperature. Another portion of freshly prepared pH 7.4 PBS solutions of CuSO₄ (0.500 mL, of 10.0 mM, 10 eq) was added and the reaction mixture was gently agitated for overnight (˜15 hours) at room temperature, then the final portion of freshly prepared pH 7.4 PBS solutions of CuSO₄ (0.500 mL, of 10.0 mM, 10 eq) was added and the reaction mixture was gently agitated for 2 hours. The reaction mixture was purified by affinity chromatography over a protein A column, followed size exclusion chromatography (See Example 164). MALDI TOF analysis of the purified final product gave an average mass of 63146 Da (DAR=2.0). Yield 25.0 mg, 83% yield.

### Example 161—Synthesis of Conjugate 41

A solution of aglycosylated hIgG1(Myc)Fc (CTP-146-2A), 3.80 mL, 0.603 μmol, 8.8 mg/mL, MW=54868 Da) in pH 7.4 PBS was treated with azido-PEG4-NHS ester (0.0019 g, 512 μL of 0.010 M solution in DMF/PBS (1:9), 5.12□μmol, 8.5 eq), then agitated gently overnight at RT. Unreacted azido-PEG4-NHS ester was removed by buffer exchange with a 10.000 MWCO centrifugal filter (6×15 mL pH 7.4 PBS). The final volume of the azide functionalized Fc was 7.5 mL with concentration at 4.30 mg/mL, average MW (MALDI)=55634, DAR (MALDI)=2.8. To this Fc-azido PBS buffer solution (0.0275 g, 0.495 μmol, 6.40 mL) were added alkyne derivatized small molecule (TFA salt, 53.4 mg, 13.9 μmol, Example 41, INT-21) and freshly prepared pH 7.4 PBS solutions of CuSO₄ (1.484 mL of 10.0 mM, 30 eq), tris(3-hydroxypropyltriazolylmethyl)-amine (THPTA, 1.484 mL of 10.0 mM, 30 eq), sodium ascorbate (2.226 mL of 10.0 mM, 45 eq). The resulting solution was agitated gently for ˜15 hours at room temperature. The reaction mixture was purified by affinity chromatography over a protein A column, followed size exclusion chromatography (See Example 164 for purification procedure). MALDI TOF analysis of the purified final product gave an average mass of 61,747 Da (DAR=2.3). Yield 17.8 mg, 65% yield.

### Example 162—Synthesis of Conjugate 47

A solution of aglycosylated hIgG1(Myc)Fc, 17.8 mL, 5.20 μmol, 16.9 mg/mL, MW=57,874 Da) in pH 7.4 PBS was treated with azido-PEG4-NHS ester (0.0019 g, 512 μL of 0.010 M solution in DMF/PBS (1:9), 5.12 μmol, 8.5 eq), then agitated gently overnight at RT. Unreacted azido-PEG4-NHS ester was removed by buffer exchange with a 10,000 MWCO centrifugal filter (6×15 mL pH 7.4 PBS). The final volume of the azide functionalized Fc was 20 mL with concentration at 14.9 mg/mL, MW (MALDI)=59303. DAR (MALDI)=5.2. To this Fc-azido PBS buffer solution (0.0300 g, 0.503 μmol, 2.00 mL) were added alkyne derivatized small molecule (TFA salt, 55.9 mg, 15.1□μmol, Example 108) and freshly prepared pH 7.4 PBS solutions of CuSO₄ (2.012 mL of 10.0 mM, 40 eq), tris(3-hydroxypropyltriazolylmethyl)-amine (THPTA, 2.012 mL of 10.0 mM, 40 eq), sodium ascorbate (3.018 mL of 10.0 mM, 60 eq). The resulting solution was agitated gently for ˜15 hours at room temperature. The reaction mixture was purified by affinity chromatography over a protein A column, followed size exclusion chromatography (See Example 164). MALDI TOF analysis of the purified final product gave an average mass of 69833 Da (DAR=4.1). Yield 26.5 mg. 88% yield.

### Example 163—Synthesis of Conjugate 52

A solution of aglycosylated BE-0096hIgG1(Myc)Fc (BE-0096, 8.50 mL, 0.603 μmol, 9.73 mg/mL, MW=54420 Da) in pH 7.4 PBS was treated with azido-PEG4-NHS ester (0.0412 g, 1056 μL of 0.010 M solution in DMF/PBS (1:9), 10.6□μmol, 7 eq), then agitated gently overnight at RT. Unreacted azido-PEG4-NHS ester was removed by buffer exchange with a 10,000 MWCO centrifugal filter (4×15 mL pH 7.4 PBS). The final volume of the azide functionalized Fc was 5.5 mL with concentration at 15.2 mg/mL, MW (MALDI)=55409. DAR (MALDI)=3.6. To this Fc-azido PBS buffer solution (0.0200 g, 0.261 μmol, 1.315 mL) were added alkyne derivatized small molecule (TFA salt, 40.4 mg, 10.5□μmol, (C-801, WJ-052-048)) and freshly prepared pH 7.4 PBS solutions of CuSO₄ (0.721 mL of 10.0 mM, 20 eq), tris(3-hydroxypropyltriazolylmethyl)-amine (THPTA, 0.721 mL of 10.0 mM, 20 eq), sodium ascorbate (1.082 mL of 10.0 mM, 30 eq). The resulting solution was agitated gently for ˜15 hours at room temperature. The reaction mixture was purified by affinity chromatography over a protein A column, followed size exclusion chromatography (See Example 164). MALDI TOF analysis of the purified final product gave an average mass of 63851 Da (DAR=3.1). Yield 18.8 mg, 94% yield.

### Example 164—ADC Purification Protocols

The crude mixtures were diluted 1:10 in PBS pH 7.4, and purified using MabSelect Sure Resin (GE Healthcare, Chicago, Ill., USA), followed by size exclusion chromatography. Purified material was quantified using a Nanodrop™ UV visible spectrophotometer (using a calculated extinction coefficient based on the amino acid sequence of h-IgG1), and concentrated to approximately 10 mg/mL using a centrifugal concentrator (10,000 MWCO). Purified molecules were analyzed using 4-12% Bis Tris SDS PAGE gels by loading 1-2 μg of each molecule into the gel, and staining using instant Blue staining. Each gel included a molecular weight ladder with the indicated molecular weight standards.

### Example 165—Efficacy of ADC Conjugate 23 in Mouse Pneumonia Model Vs. AB5075

The murine pneumonia model was adapted from Lin et al., 2015 (PMID: 26288841). A. baumannii AB5075 was grown to log-phase in LB at 37° C., 200 rpm shaking. Bacteria were washed once with PBS via centrifugation at 4,000 rpm. Bacteria were adjusted to the desired concentration in PBS.

Prior to infection, 8-week-old female C57BI/6J mice (Jackson Labs) were anesthetized with 100 mg/kg ketamine and 10 mg/kg xylazine. Using an otoscope, mice were infected non-invasively directly into the trachea.

Mice were infected with 5×10⁷ CFU in 30 μL of PBS. Mice were treated intraperitoneally with different doses of conjugate 23 or hIgG1 (50 mpk) at t=−12 h pre-infection and t=+1 h post-infection or with colistin (10 mpk) or PBS at t=+1 h, 5 h, 24 h, 36 h, 48 h and 60 h post-infection.

Survival of mice (n=5 per group) was monitored for 5 days. ADC conjugate 23 showed a dose-dependent protection: 0% protection at 1 mpk, 20% protection at 10 mpk and 80% protection at 25 and 50 mpk. Colistin was used as positive control resulting in 80% protection. In comparison, control IgG1 and PBS were not protective and mice succumbed to infection between day 1.5-2 (FIG. 95).

### Example 166—Efficacy of ADC Conjugate 34 in Mouse Pneumonia Model Vs. PAO1

The murine pneumonia model was adapted from Lin et al., 2015 (PMID: 26288841). P. aeruginosa PAO1 was grown to log-phase in LB at 37° C., 200 rpm shaking. Bacteria were washed once with PBS via centrifugation at 4,000 rpm. Bacteria were adjusted to the desired concentration in PBS. Prior to infection, 8-week-old female C57BI/6J mice (Jackson Labs) were anesthetized with 100 mg/kg ketamine and 10 mg/kg xylazine. Using an otoscope, mice were infected non-invasively directly into the trachea.

Mice were infected with 5×10⁶ CFU in 30 μL of PBS. Mice were treated intraperitoneally with conjugate 34 (25 mpk) or hIgG1 (50 mpk) at t=−12 h prior to infection or with colistin (10 mpk) or PBS at t=+1 h, 5 h, 24 h, 36 h, 48 h and 60 h post-infection.

Survival of mice (n=5 per group) was monitored for 5 days. A single dose of ADC conjugate 34 was protective (80% survival) against a lethal challenge with P. aeruginosa PAO1. Colistin was used as positive control resulting in complete protection. In comparison, control IgG1 and PBS were not protective and mice succumbed to infection between day 2-2.5 (FIG. 96).

### Example 167—Efficacy of ADC Conjugate 34 in Mouse Pneumonia Model Vs. AB5075

The murine pneumonia model was adapted from Lin et al., 2015 (PMID: 26288841). A. baumannii AB5075 was grown to log-phase in LB at 37° C., 200 rpm shaking. Bacteria were washed once with PBS via centrifugation at 4,000 rpm. Bacteria were adjusted to the desired concentration in PBS. Prior to infection, 8-week-old female C57BI/6J mice (Jackson Labs) were anesthetized with 100 mg/kg ketamine and 10 mg/kg xylazine. Using an otoscope, mice were infected non-invasively directly into the trachea.

Mice were infected with 5×10⁷ CFU in 30 μL of PBS. Mice were treated intraperitoneally with conjugate 34 or hIgG1 (50 mpk) at t=−12 h pre-infection.

Survival of mice (n=4 per group) was monitored for 5 days. A single dose of ADC conjugate 34 was protective (75% survival) against a lethal challenge with A. baumannii AB5075. Control IgG1 was not protective and mice succumbed to infection on day 2 (FIG. 97).

### Example 168—Efficacy of ADC Conjugate 23 in Mouse Pneumonia Model Vs. PAO1

The murine pneumonia model was adapted from Lin et al., 2015 (PMID: 26288841). P. aeruginosa PAO1 was grown to log-phase in LB at 37° C., 200 rpm shaking. Bacteria were washed once with PBS via centrifugation at 4,000 rpm. Bacteria were adjusted to desired concentration in PBS. Prior to infection, 8-week-old female C57BI/6J mice (Jackson Labs) were anesthetized with 100 mg/kg ketamine and 10 mg/kg xylazine. Using an otoscope, mice were infected non-invasively directly into the trachea.

Mice were infected with 5×10⁸ CFU in 30 μL of PBS. Mice were treated intraperitoneally with conjugate 23 or hIgG1 (50 mpk) at t=−12 h prior to infection or with colistin (10 mpk) or PBS at t=+1 h and 5 h post-infection.

Survival of mice (n=5 per group) was monitored for 5 days. A single dose of ADC conjugate 23 was completely protective against challenge with P. aeruginosa PAO1. Positive control, colistin, was also protective (100%). Controls IgG1 (60% survival) and PBS (20% survival) were only partially protective (FIG. 98).

### Example 169—Efficacy of ADC Conjugate 23 in Mouse Pneumonia Model Vs. PAO1

The murine pneumonia model was adapted from Lin et al., 2015 (PMID: 26288841). P. aeruginosa PAO1 was grown to log-phase in LB at 37° C., 200 rpm shaking. Bacteria were washed once with PBS via centrifugation at 4,000 rpm. Bacteria were adjusted to the desired concentration in PBS. Prior to infection, 8-week-old female C57BI/6J mice (Jackson Labs) were anesthetized with 100 mg/kg ketamine and 10 mg/kg xylazine. Using an otoscope, mice were infected non-invasively directly into the trachea.

Mice were infected with 5×10⁶ CFU in 30 μL of PBS. Mice were treated intraperitoneally with conjugate 23, 34, 35, 38, 44 or hIgG1 (25 mpk) at t=−12 h prior to infection or with colistin (10 mpk) or PBS at t=+1 h and 5 h post-infection.

Survival of mice (n=5 per group) was monitored for 5 days. Single doses of ADC conjugates 23, 34, 35, 38 or 44 were completely protective against challenge with P. aeruginosa PAO1. Positive control, colistin, was also protective (100%). The IgG1 control (60% survival) was only partially protective and mice in the PBS group all succumbed to infection on day 2 (FIG. 99).

### Example 170—Protective Effect of Conjugate 23 Against Endotoxemia

Septic shock, resulting in systemic organ failure, is an important component of many bacterial infections. In infections caused by Gm-negative pathogens, or in some mixed infections, LPS (aka endotoxin) is the dominate mediator of this process which results from hyper-stimulation of the immune system. In a mouse model of endotoxemia we were able to demonstrate the protective effect of Conjugate 23 to rescue mice given a lethal dose of LPS.

Mouse model of endotoxemia. These studies used female immune competent C57BL/6 mice between 8 and 10 weeks old. After acclimation for 7 days mice were challenged IP with 12.5 mg/kg of purified LPS (Escherichia coli 0111:B4) in PBS from Sigma (#L4130) in a volume of 0.2 ml. Previously this dose was shown to result in 100% mortality of challenged mice within 5 days. Study mice were housed and cared for following standard IACUC approved protocols, and clearly moribund mice were humanely euthanized and scored as a death.

Efficacy of Conjugate 23 in endotoxemia. C57BL/6 mice were challenged with a lethal dose of LPS as described above. Immediately following LPS challenge 2 groups of animals received vehicle (PBS) or colistin (10 mg/kg) as a separate IP injection. Due to the short half-life of colistin both groups were dosed twice daily (bid, separated by 4-6 hours) for 2 days. In contrast, conjugate 23 was administered as a single IP dose at 25 mg/kg 4 hours prior to LPS challenge. The F_{c}—only control for conjugate 23 which lacks the LPS recognition moiety was dosed following the same procedure (hIgG1).

As illustrated in FIG. 100, mice dosed with the vehicle or hIgG1 reached 100% mortality within 48 hours of LPS challenge. Mice receiving multiple doses of colistin were partially protected with ˜30% of animals reaching death after 5 days. In contrast, all mice treated with conjugate 23 were rescued with 0% mortality. This study demonstrates the LPS neutralizing capabilities of this class of compounds and suggests an additional mechanism of action beyond direct killing of bacteria.

### Example 171—Conjugate Activity in Human Blood

Overnight stationary-phase cultures were centrifuged at 3,500×g for 10 min at room temperature and resuspended in 1×PBS to an optical density at 600 nm (OD₆₀₀) of 0.4 (1×10⁸ colony forming units [CFU]/ml) in RPMI with L-glutamine and phenol red (Life Technologies). Bacteria were diluted 1:25 in RPMI (4×10⁸ CFU/ml) and 10 ul added to wells of a non-binding 96-well plate (Corning 3641) containing 80 ul of 50% hirudin anti-coagulated blood (complement active) in RPMI. After adding 5 ul of 20× conjugate diluted in RPMI (128-2 ug/ml final concentration), and 5 ul RPMI for a final blood concentration of 40% in 100 ul, the plate was incubated under static conditions at 37° C. for 90 min. The minimum bactericidal concentration (MBC) was determined by 10-fold serial dilution in 1×PBS, then plating 4 ul spots onto rectangular LB agar plates for overnight incubation at 37° C. MBC was defined as the minimum conjugate concentration required for bacterial clearance (Table 8A and Table 8B). An untreated growth control (40% blood, 0 ug/ml conjugate) was included in all assays as a growth comparator.

TABLE 8A
MBC values of conjugates in blood

| | MBC (μg/mi) in human hirudin- | | | |
|---|---|---|---|---|
| | anticoagulated blood | | | |
| | E. coli | A. | P. | P. |
| | ATCC | baumannii | aeruginosa | aeruginosa |
| Compound | 25922 | AB5075 | PAO1 | PA7874 |
| hlgG1 Fc | >128 | >128 | >128 | >128 |
| | >128 | >128 | >128 | >128 |
| | >128 | >128 | >128 | >128 |
| Conjugate 21 | 6 | 6 | >52 | >52 |
| Conjugate 17 | 5 | 1.8 | >49 | 49 |
| Conjugate 22 | 4 | ≤2 | >128 | 64 |
| | 4 | 4 | >32 | >32 |
| Conjugate 23 | 50.5 | 1 | 32 | 32 |
| | 4 | 2 | 64 | |
| Conjugate 24 | 16 | <2 | >128 | 128 |
| | 8 | <2 | >128 | >128 |
| | 8 | 2 | >32 | >32 |
| Conjugate 25 | 2 | ≤2 | 32 | 32 |
| | ≤2 | ≤2 | 64 | 32 |
| | 1 | ≤0.5 | >32 | 16 |
| Conjugate 27 | 32 | 32 | >32 | >32 |
| Conjugate 28 | 16 | 2 | >32 | 32 |
| Conjugate 29 | 32 | >32 | >32 | >32 |
| Conjugate 30 | 2 | 1 | >32 | 32 |
| Conjugate 31 | 2 | 2 | >32 | 32 |
| Conjugate 32 | >32 | 16 | >32 | >32 |
| Conjugate 33 | 8 | 16 | >32 | >32 |
| Conjugate 34 | ≤0.5 | 0.5 | 4 | 4 |
| | 0.5 | ≤0.5 | 16 | 8 |
| | 0.5 | ≤0.5 | 8 | 8 |
| | 1 | 1 | 8 | 8 |
| Conjugate 35 | 1 | 1 | >32 | >32 |
| Conjugate 36 | 16 | 4 | >32 | >32 |
| Conjugate 37 | 16 | 4 | >32 | >32 |
| Conjugate 38 | 2 | 1 | >32 | 32 |
| | 1 | 1 | 32 | 16 |
| Conjugate 39 | 1 | 1 | 32 | 16 |
| | 1 | 2 | 16 | 8 |
| Conjugate 40 | ≤0.5 | ≤0.5 | 16 | 8 |
| | ≤0.5 | ≤0.5 | 8 | 8 |
| Conjugate 41 | 2 | 2 | >32 | 32 |
| | 2 | 2 | >32 | 16 |
| Conjugate 42 | 32 | 8 | >32 | >32 |
| | 32 | 8 | >32 | >32 |
| Conjugate 43 | 4 | 8 | >32 | 32 |
| | 4 | 16 | >32 | >32 |
| Conjugate 44 | 4 | 1 | >32 | 32 |
| | 1 | 2 | 32 | 16 |

TABLE 8B
MBC values of conjugates in blood

| | MBC (μg/ml) in human hirudin- | | | |
|---|---|---|---|---|
| | anticoagulated blood | | | |
| | E. coli | A. | P. | P. |
| | ATCC | baumannii | aeruginosa | aeruginosa |
| Compound | 25922 | AB5075 | PAO1 | PA7874 |
| Conjugate 45 | >32 | 16 | >32 | >32 |
| | 16 | 32 | >32 | >32 |
| Conjugate 46 | 16 | 16 | >32 | >32 |
| | 32 | 32 | >32 | >32 |
| Conjugate 47 | 8 | 4 | >32 | >32 |
| | 4 | 4 | >32 | >32 |
| Conjugate 48 | 2 | 2 | >32 | >32 |
| | 2 | 2 | >32 | 32 |
| Conjugate 49 | 2 | 2 | >32 | 32 |
| Conjugate 50 | 16 | 8 | >32 | >32 |
| Conjugate 51 | 4 | 2 | >32 | 32 |
| Conjugate 52 | 4 | 4 | >32 | 32 |
| Conjugate 53 | 2 | 2 | >32 | 32 |
| Conjugate 54 | 32 | 16 | >32 | >32 |
| Conjugate 55 | 32 | 16 | >32 | >32 |
| Conjugate 56 | 4 | 8 | >32 | 32 |
| Conjugate 57 | 2 | 2 | >32 | 32 |
| Conjugate 58 | 8 | 4 | >32 | >32 |
| Conjugate 59 | 2 | 2 | >32 | >32 |
| Conjugate 60 | 4 | 4 | >32 | >32 |
| Conjugate 61 | 8 | 4 | >32 | NT |
| Conjugate 62 | 16 | 4 | >32 | NT |
| Conjugate 63 | >32 | 16 | >32 | NT |
| Conjugate 64 | 16 | 8 | >32 | NT |
| Conjugate 65 | 16 | 8 | >32 | NT |
| Conjugate 66 | 32 | 8 | >32 | NT |
| Conjugate 67 | 32 | 16 | >32 | NT |
| Conjugate 68 | 16 | 4 | >32 | NT |
| Conjugate 69 | 16 | 8 | >32 | NT |
| Conjugate 70 | 32 | >32 | >32 | NT |
| Conjugate 71 | 32 | 32 | >32 | NT |
| Conjugate 72 | 4 | <2 | 128 | 64 |
| Conjugate 73 | 4 | 2 | 128 | 64 |
| Conjugate 74 | 8 | 8 | >32 | NT |
| Conjugate 75 | 16 | 4 | >32 | NT |

### Example 172—Conjugate Activity in Human Serum

Overnight stationary-phase cultures were centrifuged at 3,500×g for 10 min at room temperature and resuspended in 1×PBS to an optical density at 600 nm (OD₆₀₀) of 0.4 (1×10⁸ colony forming units [CFU]/m1 in RPMI with L-glutamine and phenol red (Life Technologies). Bacteria were diluted 1:25 in RPMI (4×10⁶ CFU/ml) and 10 uL added to wells of a non-binding 96-well plate (Corning 3841) containing 80 ul of 50% normal human serum (complement-active; Innovative Research IPLA-CSER) in RPMI, or 50% heat-inactivated human serum (56° C. for 30 min; complement inactive) in RPMI. After adding 5 ul of 20× conjugate diluted in RPMI (128-2 ug/ml final concentration), and 5 ul RPMI for a final serum concentration of 40% in 100 ul, the plate was incubated under static conditions at 37° C. for 90 min. The minimum bactericidal concentration (MBC) was determined by 10-fold serial dilution in 1×PBS, then plating 4 ul spots onto rectangular LB agar plates for overnight incubation at 37° C. MBC was defined as the minimum conjugate concentration required for bacterial clearance (Table 9). An untreated growth control (40% serum, 0 ug/ml conjugate) was included in all assays as a growth comparator. MBCs are typically lower in complement active normal human serum compared to complement inactive heat-inactivated human serum, indicating that conjugate-mediated killing is complement dependent (Table 9).

TABLE 9
MBC values of conjugates in serum
MBC (μg/ml) in normal human serum (heat-
inactivated human serum)

| | A. baumannii | | P. aeruginosa | |
|---|---|---|---|---|
| | AB5075 | | PAO1 | |
| Conjugate 46 | 32 | (>32) | >32 | (>32) |
| Conjugate 47 | 8 | (16) | >32 | (>32) |
| Conjugate 48 | 8 | (16) | >32 | (>32) |
| Conjugate 49 | 4 | (8) | >32 | (>32) |
| Conjugate 50 | 8 | (32) | >32 | (>32) |
| Conjugate 51 | 4 | (8) | >32 | (>32) |
| Conjugate 52 | 2 | (8) | >32 | (>32) |
| Conjugate 53 | 4 | (8) | >32 | (>32) |
| Conjugate 54 | 32 | (>32) | >32 | (>32) |
| Conjugate 55 | 32 | (>32) | >32 | (>32) |
| Conjugate 56 | 8 | (32) | >32 | (>32) |
| Conjugate 57 | 4 | (32) | >32 | (>32) |
| Conjugate 58 | 8 | (32) | >32 | (>32) |
| Conjugate 59 | 16 | (16) | >32 | (>32) |
| Conjugate 60 | 16 | (32) | >32 | (>32) |
| Conjugate 72 | 4 | (16) | >128 | (>128) |
| Conjugate 73 | 4 | (32) | >128 | (>128) |

### Example 173—In Vitro Neutrophil Killing Assay

5×10⁴ log phase E. coli ATCC 25922 were added to 96-well, round-bottom, non-treated tissue culture plates in the presence or absence of 25 nM ADC. 5×10⁴ polymorphprep (AXIS-SHIELD) purified human neutrophils were added to a portion of the wells and plates were incubated at 37° C. shaking at 800 rpm for the indicated time points. Aliquots were removed at each time point, serially diluted in water to lyse neutrophils and incubated overnight on agar plates at 37° C. Surviving CFU/ml were calculated by manually counting colonies and data were graphed using the following formula: % of inoculum=([CFU/mL of test condition]/[CFU/mL of inoculum])*100 (FIG. 101).

### Example 174—Neutrophil Phagocytosis of FITC-Labeled E. coli (K-12) BioParticles

10⁵ FITC-labeled E. coli BioParticles (Invitrogen) were opsonized with 250 nM ADC or media only by co-incubation for 25 min at 37° C. shaking (650 rpm). 2×10⁵ polymorphprep purified human neutrophils were then added and plates were incubated at 37° C. shaking at 650 rpm for 10 min. Neutrophils were placed on ice for 5 minutes to stop phagocytosis and then washed in cold PBS followed by fixation in 2% PFA. Neutrophils were incubated in 0.2% trypan blue for 2 minutes to quench extracellular fluorescence, washed once and analyzed on a FACSCalibur (BD Biosciences) in the presence of residual trypan blue. Fluorescence was measured in the Blu FL1 channel (FIG. 102).

### Example 175—Synthesis of Int-74

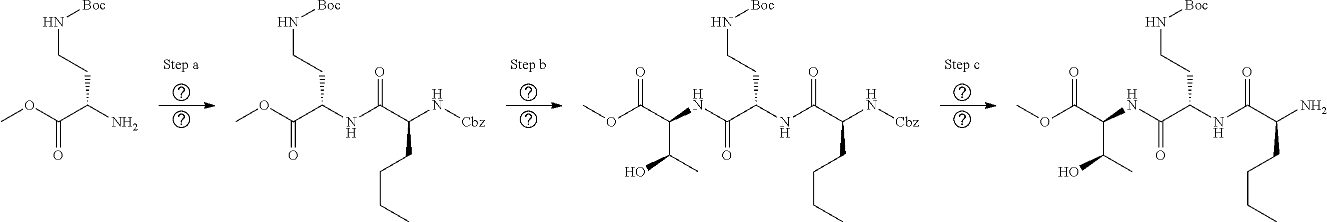 [SMILES: COC(=O)[C@@H](N)CCNC(=O)OC(C)(C)C ; CCCC[C@H](NC(=O)OCc1ccccc1)C(=O)N[C@@H](CCNC(=O)OC(C)(C)C)C(=O)OC ; CCCC[C@H](NC(=O)OCc1ccccc1)C(=O)N[C@@H](CCNC(=O)OC(C)(C)C)C(=O)N[C@H](C(=O)OC)[C@@H](C)O ; CCCC[C@H](N)C(=O)N[C@@H](CCNC(=O)OC(C)(C)C)C(=O)N[C@H](C(=O)OC)[C@@H](C)O]

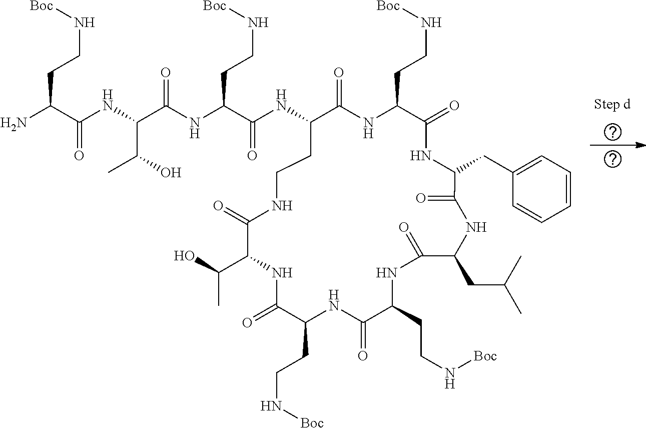

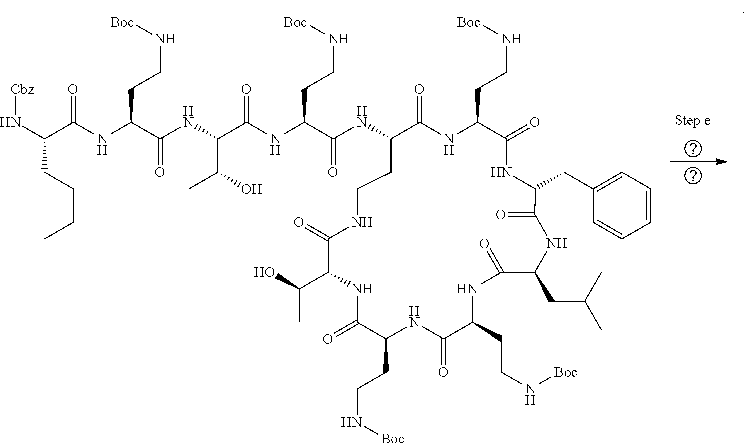

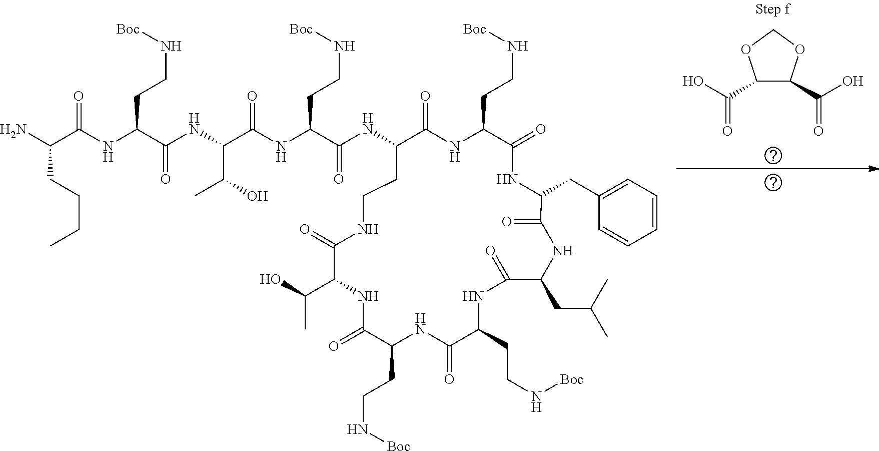

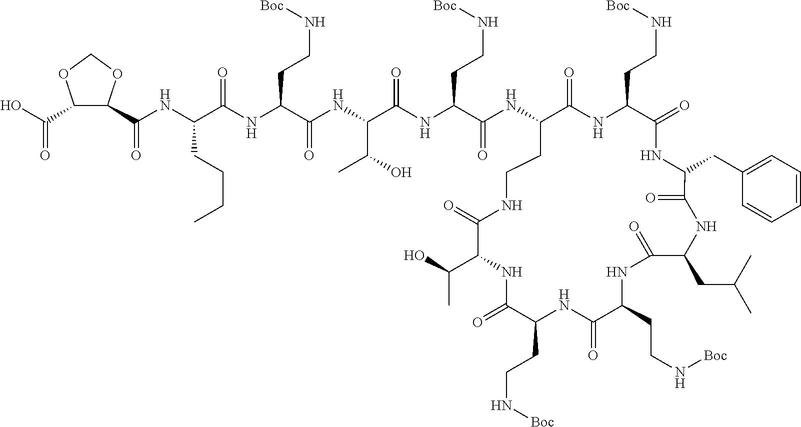

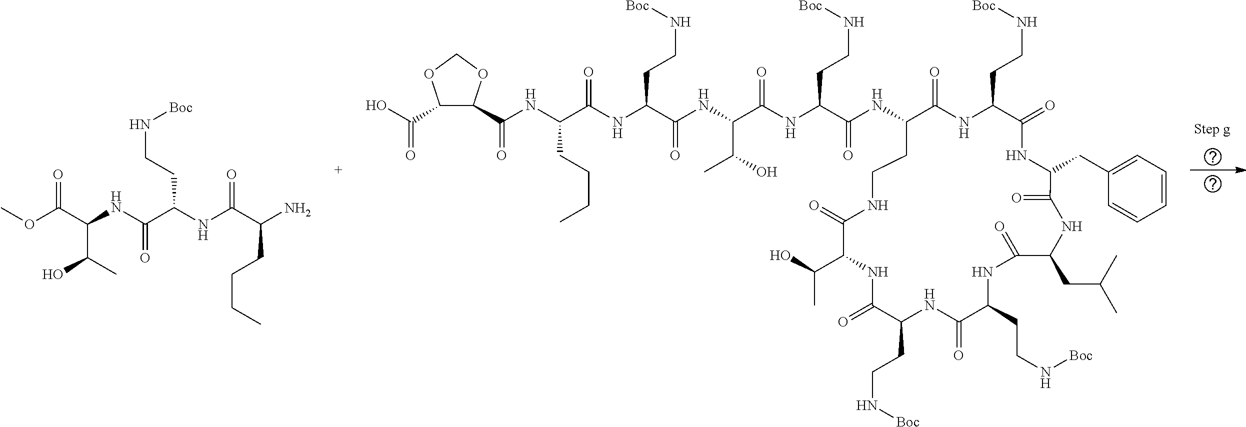

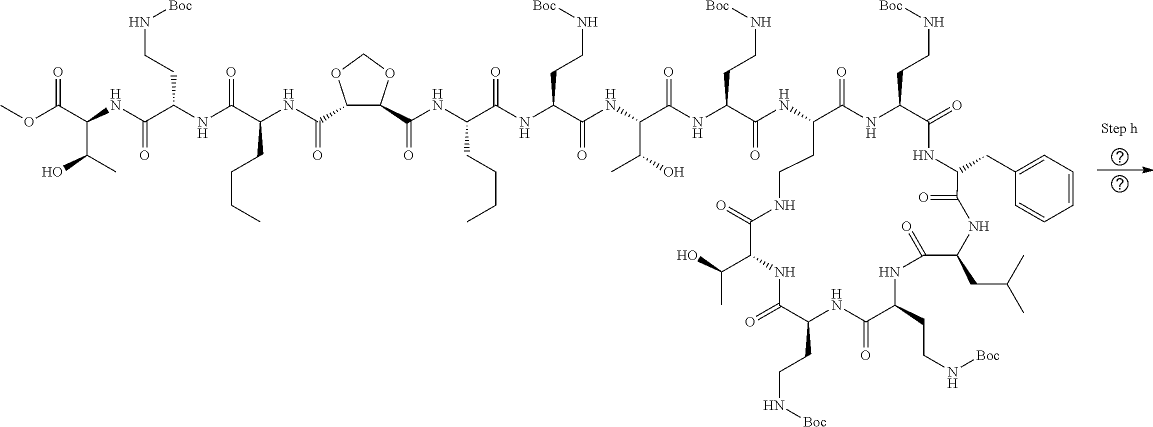

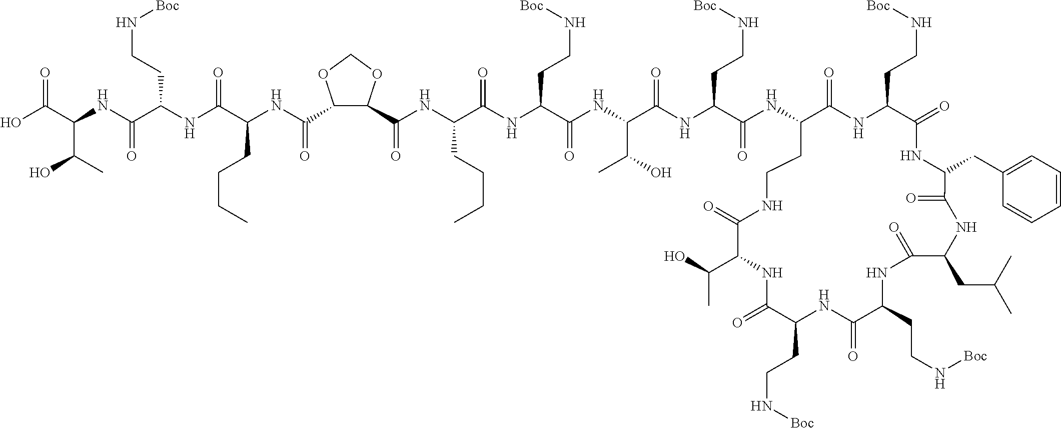

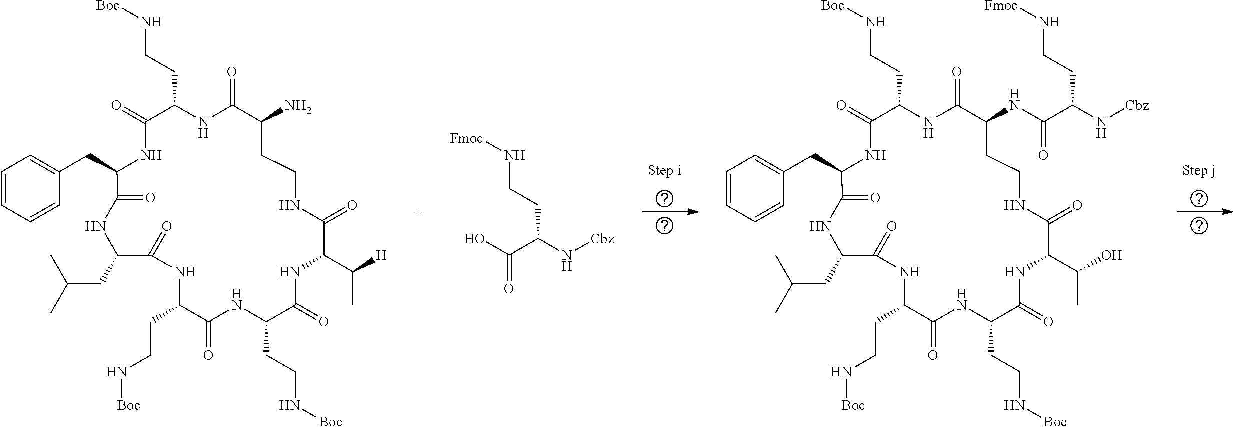

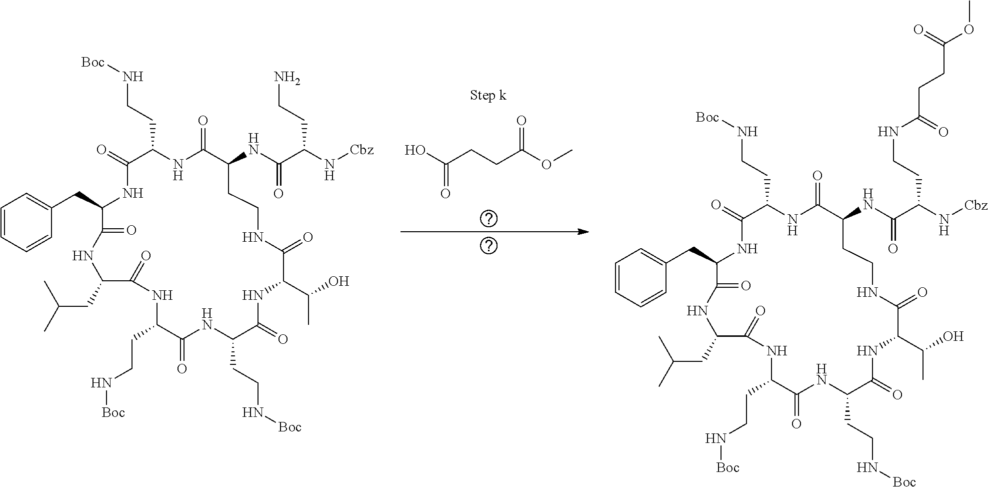

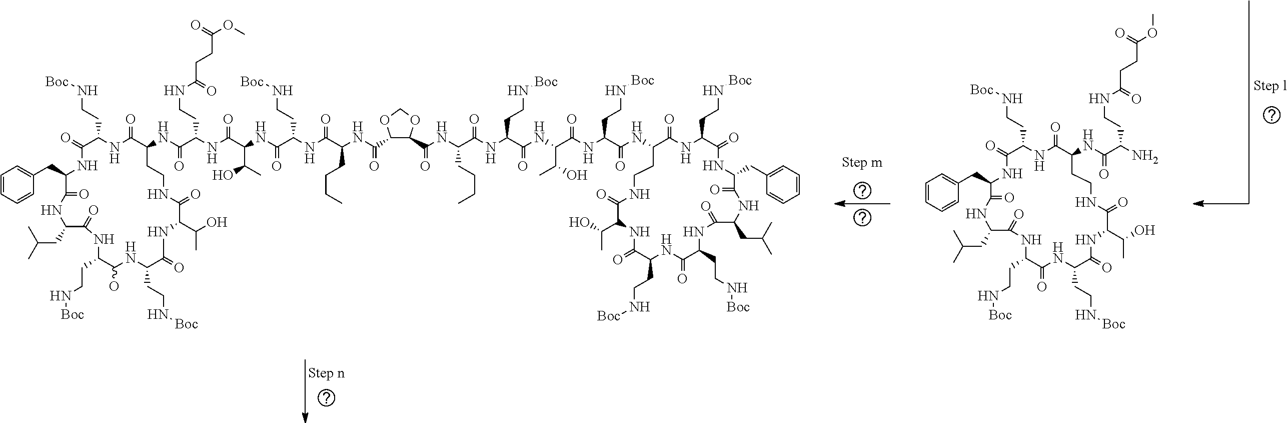

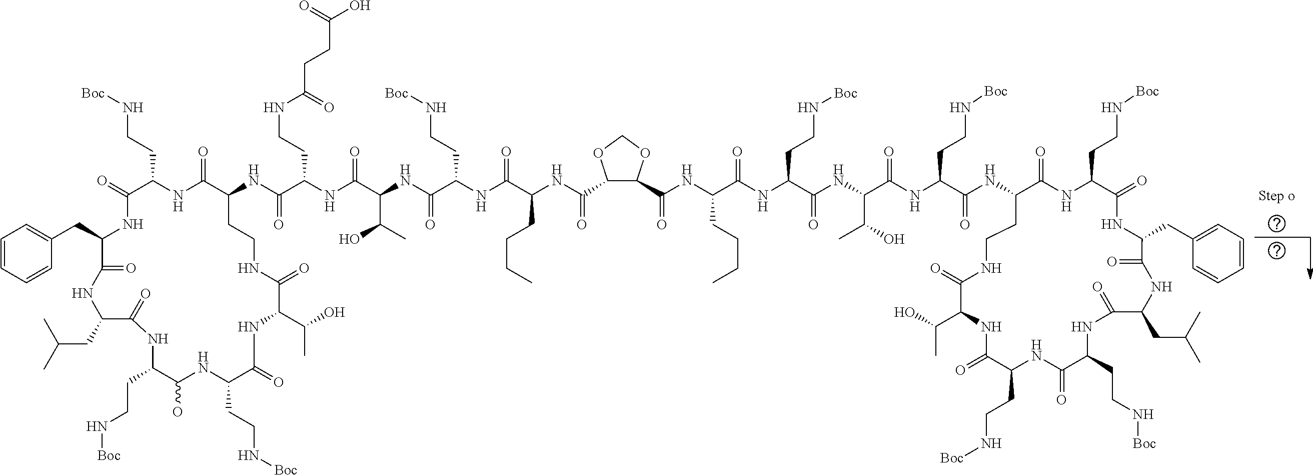

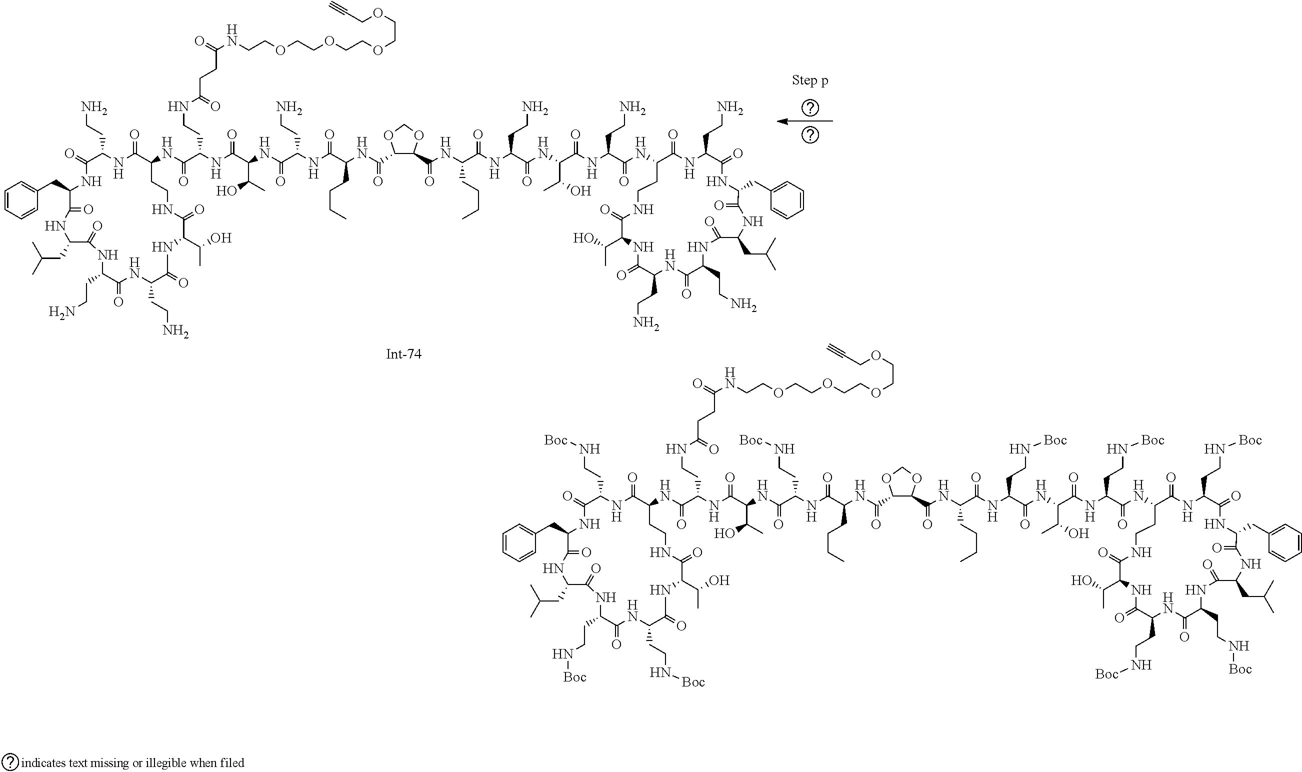

### Step a

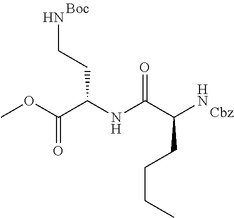

To a 0° C. stirring solution of Nγ-Boc-L-2,4-diaminobutyric acid methyl ester hydrochloride (0.750 g, 2.791 mmol), L-Z-Nleu-OH (0.740 g, 2.791 mmol), and 2,4,6-collidine (1.217 mL, 9.210 mmol) in DMF (5.0 mL), was added HATU (1.082 g, 2.847 mmol). Temperature was slowly allowed to reach ambient temperature over 15 min. Upon completion, all the volatiles were removed by rotary evaporation. The residue was purified by RPLC using an Isco CombiFlash liquid chromatograph eluted with 25% to 100% water and methanol, using no modifier. Yield 1.230 g, 92% yield of Z-Nor-leu-(Nγ-Boc)Dab-OMe. ¹H NMR (Methanol-d₄) δ: 7.49-7.20 (m, 5H), 5.09 (br s, 2H), 4.46 (dd, J=9.4, 4.8 Hz, 1H), 4.09 (dd, J=8.5, 5.7 Hz, 1H), 3.71 (s, 3H), 3.26-2.93 (m, 2H), 2.15-1.92 (m, 1H), 1.90-1.54 (m, 3H), 1.43 (s, 11H), 1.36 (d, J=8.1 Hz, 4H), 0.94 (br d, J=6.7 Hz, 3H).

### Step b

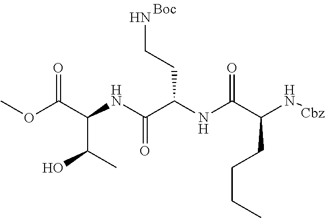 [SMILES: CCCC[C@H](NC(=O)OCc1ccccc1)C(=O)N[C@@H](CCN)C(=O)N[C@H](C(=O)OC)[C@@H](C)O]

To a stirring solution of Z-Nleu-(Nγ-Boc)Dab-OMe (0.123 g, 2.565 mmol) in tetrahydrofuran (15.0 mL) and water (5.0 mL) it was added lithium hydroxide (0.064 g, 2.693 mmol). Upon completion (HPLC monitoring), the reaction was concentrated to about 5 mL per rotatory evaporation. Ethyl acetate (25 mL) was added and subsequently a 1.0 M solution of sulfuric acid (5.0 mL), while the mixture was stirred vigorously for 10 minutes. The organic phase was separated and then washed with water (10 mL) and brine (10 mL). The organic layer was then dried with sodium sulfate, filtered, and concentrated to dryness by rotary evaporation. The residue was dissolved in DMF (15 mL) with L-Threonine methyl ester hydrochloride (0.435 g, 2.565 mmol), and 2,4,6-collidine (1.356 mL, 10.259 mmol). At 0° C., HATU (0.995, 2.616 mmol) was added. Temperature was slowly allowed to reach ambient temperature over 15 min. Upon completion, all the volatiles were removed by rotary evaporation. The residue was purified by RPLC using an Isco CombiFlash liquid chromatograph eluted with 25% to 100% water and methanol, using no modifier. Yield 1.398 g, 94% yield of Z-Nleu-(Nγ-Boc)Dab-Thr-OMe. ¹H NMR (Methanol-d₄) δ: 7.46-7.19 (m, 5H), 5.09 (s, 2H), 4.56-4.38 (m, 2H), 4.30 (m, 1H), 4.10 (m, 1H), 3.74 (s, 3H), 3.27-2.98 (m, 2H), 2.12-1.53 (m, 4H), 1.44 (s, 10H), 1.34 (br s, 4H), 1.17 (d, J=6.4 Hz, 3H), 0.91 (br t, J=6.6 Hz, 3H).

### Step c

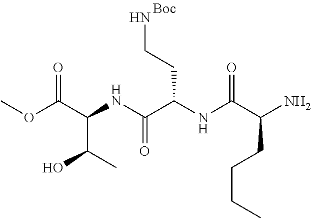 [SMILES: CCCC[C@H](N)C(=O)N[C@@H](CCN)C(=O)N[C@H](C(=O)OC)[C@@H](C)O]

Z-Nleu-(Nγ-Boc)Dab-Thr-OMe (1.398 g, 2.408 mmol) was dissolved in methanol (25 mL) and suspended with SiliaCatPd(0)® (0.241 g, 0.048 mmol) under hydrogen atmosphere (˜1 atm). When all the starting material was consumed by HPLC analysis, the mixture was filtered over a short Celite pad, and all the volatiles were removed by rotary evaporation. HPLC analysis for the residues revealed one component in high purity, so it was used in the next step without further purification. Yield 1.075 g, 99% yield of H-Nleu-(Nγ-Boc)Dab-Thr-OMe. ¹H NMR (Methanol-d₄) δ: 4.49 (dd, J=8.8, 5.8 Hz, 1H), 4.45 (d, J=2.8 Hz, 1H), 4.31 (qd, J=6.4, 3.0 Hz, 1H), 3.74 (s, 3H), 3.36 (d, J=6.7 Hz, 1H), 3.26-3.01 (m, 2H), 2.08-1.49 (m, 5H), 1.44 (s, 9H), 1.41-1.29 (m, 4H), 1.17 (d, J=6.4 Hz, 3H), 0.92 (t, J=7.0 Hz, 3H)

### Step d

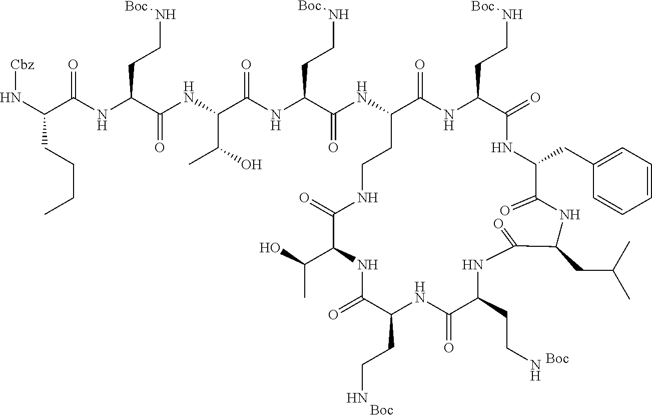 [SMILES: CCCC[C@H](NC(=O)OCc1ccccc1)C(=O)N[C@@H](CCNC(=O)OC(C)(C)C)C(=O)N[C@H](C(=O)N[C@@H](CCNC(=O)OC(C)(C)C)C(=O)N[C@H]1CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](Cc2ccccc2)NC(=O)[C@H](CCNC(=O)OC(C)(C)C)NC1=O)[C@@H](C)O]

To a 0° C. stirring solution of Int-7a (2.50 g, 1.599 mmol, described in Example 8 step a), L-Z-Nleu-OH (0.424 g, 1.599 mmol), and 2,4,6-collidine (0.274 mL, 2.078 mmol) in DMF (20 mL), it was added HATU (0.638 g, 1.679 mmol) in DMF (5.0 mL), dropwise. Temperature was slowly allowed to reach ambient temperature over 15 min. Upon completion, all the volatiles were removed by rotary evaporation. The residue was purified by RPLC using an Isco CombiFlash liquid chromatograph eluted with 50% to 100% water and methanol, using no modifier. Yield 2.720 g, 94% yield.

### Step e

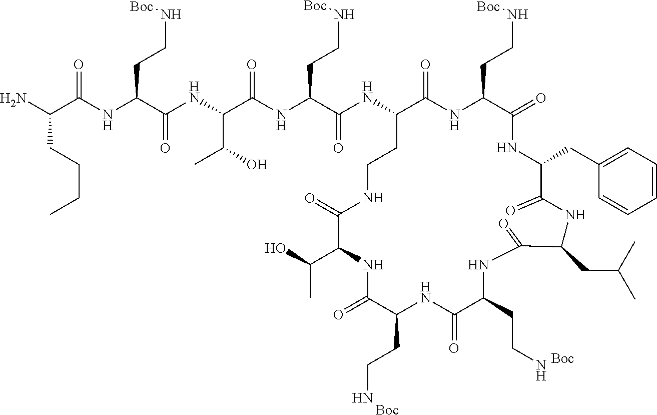 [SMILES: CCCC[C@H](N)C(=O)N[C@@H](CCNC(=O)OC(C)(C)C)C(=O)N[C@H](C(=O)N[C@@H](CCNC(=O)OC(C)(C)C)C(=O)N[C@H]1CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](Cc2ccccc2)NC(=O)[C@H](CCNC(=O)OC(C)(C)C)NC1=O)[C@@H](C)O]

Product from step d (2.720 g, 1.502 mmol) was dissolved in methanol (100 mL) and suspended with SiliaCatPd(0)® (0.751 g, 0.150 mmol) under hydrogen atmosphere (˜1 atm). When all the starting material was consumed by HPLC analysis, the mixture was filtered over a short Celite pad, and all the volatiles were removed by rotary evaporation. Yield 1.075 g, 99% yield. ¹H NMR for the diagnostic peaks: ¹H NMR (Methanol-d₄) δ: 7.39-7.09 (m, 5H), 4.58-3.92 (m, 13H), 3.52 (br s, 1H), 1.44 (br d, J=4.6 Hz, 45H), 1.20 (dd, J=6.6, 3.7 Hz, 6H), 1.02-0.83 (m, 3H), 0.69 (br d, J=19.8 Hz, 6H).

### Step f

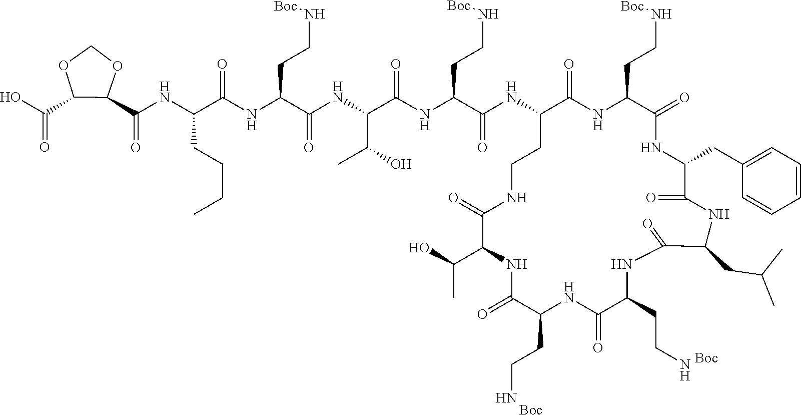 [SMILES: CCCC[C@H](NC(=O)[C@@H]1OCO[C@H]1C(=O)O)C(=O)N[C@@H](CCNC(=O)OC(C)(C)C)C(=O)N[C@H](C(=O)N[C@@H](CCNC(=O)OC(C)(C)C)C(=O)N[C@H]1CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](Cc2ccccc2)NC(=O)[C@H](CCNC(=O)OC(C)(C)C)NC1=O)[C@@H](C)O]

To a 0° C. stirring solution of product from step g (0.300 g, 0.177 mmol), 2,3-O-methylene-L-tartaric acid (0.058 g, 0.354 mmol, described in PCT Int. Appl., 2007139749), and 2,4,6-collidine (0.070 mL, 0.532 mmol) in DMF (3.0 mL), was added HATU (0.069 g, 0.181 mmol) in DMF (1.5 mL), dropwise. Temperature was slowly allowed to reach ambient temperature over 15 min. Upon completion, all the volatiles were removed by rotary evaporation. The residue was purified by RPLC using an Isco CombiFlash liquid chromatograph eluted with 50% to 100% water and methanol, using no modifier. Yield 0.212 g, 66% yield. ¹H NMR for the diagnostic peaks: ¹H NMR (Methanol-d₄) δ: 7.40-7.17 (m, 5H), 5.29 (s, 1H), 5.14 (s, 1H), 4.74 (d, J=3.8 Hz, 1H), 4.68 (d, J=3.8 Hz, 1H), 4.55-3.97 (m, 13H), 3.54 (br s, 1H), 1.44 (br s, 45H), 1.20 (br d, J=6.4 Hz, 7H), 0.92 (br s, 3H), 0.70 (br d, J=19.4 Hz, 6H).

### Step g

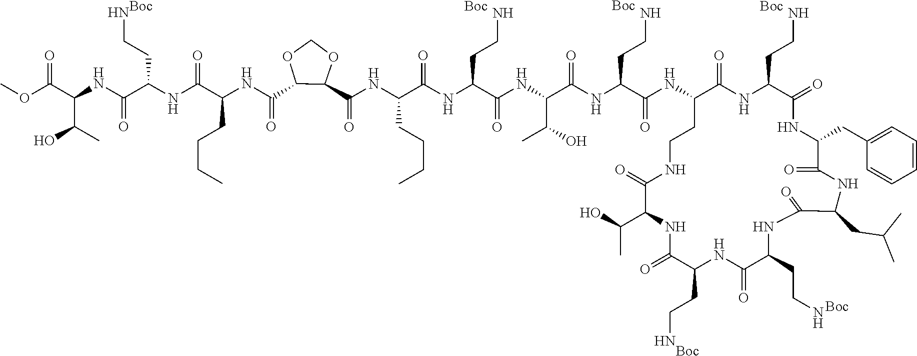 [SMILES: CCCC[C@H](NC(=O)[C@@H]1OCO[C@H]1C(=O)N[C@@H](CCCC)C(=O)N[C@@H](CCNC(=O)OC(C)(C)C)C(=O)N[C@H](C(=O)OC)[C@@H](C)O)C(=O)N[C@@H](CCNC(=O)OC(C)(C)C)C(=O)N[C@H](C(=O)N[C@@H](CCNC(=O)OC(C)(C)C)C(=O)N[C@H]1CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](Cc2ccccc2)NC(=O)[C@H](CCNC(=O)OC(C)(C)C)NC1=O)[C@@H](C)O]

To a 0° C. stirring solution of product from step f (0.212 g, 0.116 mmol), H-Nleu-(γ-Boc)Dab-Thr-OMe (0.052 g, 0.116 mmol), and DIEA (0.051 mL, 0.291 mmol) in DMF (3.0 mL), it was added HATU (0.045 g, 0.119 mmol). Temperature was slowly allowed to reach ambient temperature over 15 min. Upon completion, all the volatiles were removed by rotary evaporation. The residue was purified by RPLC using an Isco CombiFlash liquid chromatograph eluted with 50% to 100% water and methanol, using no modifier. Yield 0.214 g, 82% yield. ¹H NMR for the diagnostic peaks: ¹H NMR (Methanol-d₄) δ: 7.36-7.17 (m, 5H), 5.22 (d, J=7.6 Hz, 2H), 4.66 (br s, 2H), 4.57-3.96 (m, 17H), 3.74 (s, 3H), 3.54 (br s, 1H), 1.44 (s, 54H), 1.26-1.07 (m, 9H), 1.03-0.82 (m, 6H), 0.70 (br d, J=18.0 Hz, 6H).

### Step h

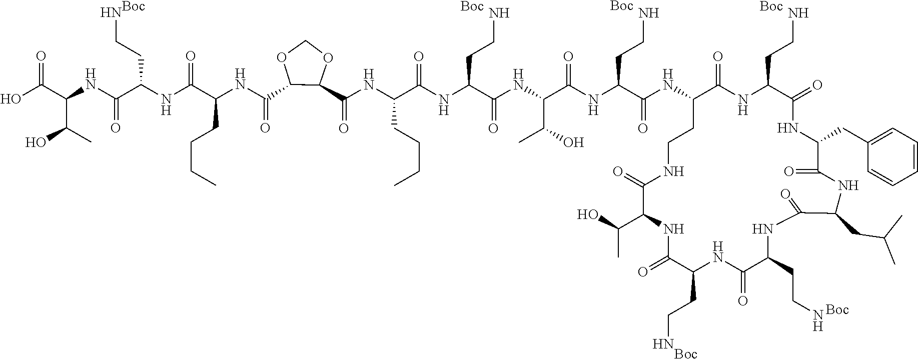 [SMILES: CCCC[C@H](NC(=O)[C@@H]1OCO[C@H]1C(=O)N[C@@H](CCCC)C(=O)N[C@@H](CCNC(=O)OC(C)(C)C)C(=O)N[C@H](C(=O)N[C@@H](CCNC(=O)OC(C)(C)C)C(=O)N[C@H]1CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](Cc2ccccc2)NC(=O)[C@H](CCNC(=O)OC(C)(C)C)NC1=O)[C@@H](C)O)C(=O)N[C@@H](CCNC(=O)OC(C)(C)C)C(=O)N[C@H](C(=O)O)[C@@H](C)O]

Product from step g (0.1 mmol) is taken up in 1,2-dichloroethane (5.0 mL), treated with trimethyltin hydroxide (1.0 mmol), and the mixture is refluxed until completion. Upon cooling, the mixture is treated under stirring with SiliaMetS® (2.0 mmol) for 1 h. All the volatiles are removed by rotary evaporation. The residue is taken up in acetic acid (5 mL) and filtered over a short celite pad. All the volatiles are removed by rotary evaporation, to afford the desired product.

### Step i

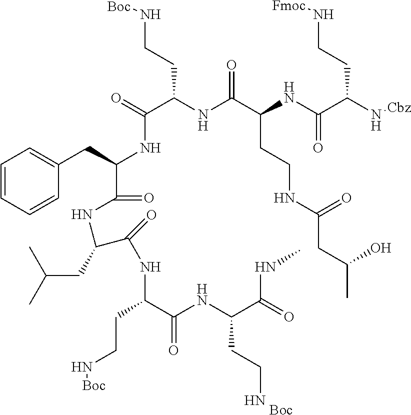

To a 0° C. stirring solution of tri-Boc polymyxin B cycloheptapeptide (0.400 g, 0.376 mmol, described at Example 2 as Int-1), Nα-Z-Nγ-Fmoc-L-2,4-diaminobutyric acid (0.179 g, 0.376 mmol), and 2,4,6-collidine (0.104 mL, 0.791 mmol) in DMF (5.0 mL), was added HATU (0.150 g, 0.395 mmol) in DMF (1.5 mL), dropwise. Temperature was slowly allowed to reach ambient temperature over 15 min. Upon completion, all the volatiles were removed by rotary evaporation. The residue was purified by RPLC using an Isco CombiFlash liquid chromatograph eluted with 50% to 100% water and methanol, using no modifier. Yield 0.526 g, 92% yield. ¹H NMR for the diagnostic peaks: ¹H NMR (Methanol-d₄) δ: 7.79 (d, J=7.5 Hz, 2H), 7.64 (d, J=7.5 Hz, 2H), 7.52-7.11 (m, 16H), 5.18-4.98 (m, 2H), 3.54 (br s, 1H), 1.67-1.25 (m, 27H), 1.18 (d, J=6.4 Hz, 3H), 0.70 (br d, J=18.8 Hz, 6H)

### Step j

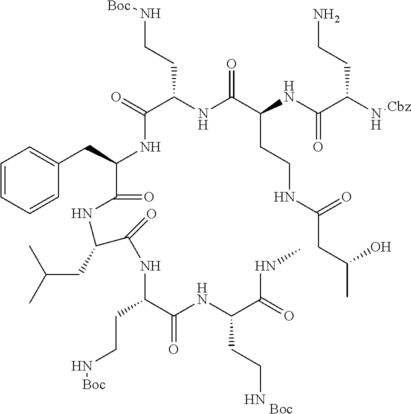

To a stirring solution of product from step i (0.526 mmol, 0.346 mmol), 1-octanethiol (0.090 mL, 0.520 mmol) in dichloromethane (5.0 mL), it was added 1,8-diazabicyclo[5.4.0]undec-7-ene (0.057 mL, 0.381 mmol). Upon completion, all the volatiles were removed by rotary evaporation. The residue was purified by RPLC using an Isco CombiFlash liquid chromatograph eluted with 50% to 100% water and methanol, using 0.1% TFA as the modifier. Yield 0.370 g, 82% yield. ¹H NMR for the diagnostic peaks: ¹H NMR (300 MHz, MeOD) δ 7.58-7.10 (m, 10H), 5.10 (q, J=12.4 Hz, 2H), 3.56 (br s, 1H), 1.45 (br s, 27H), 1.21 (d, J=6.5 Hz, 3H), 0.72 (br d, J=19.1 Hz, 6H).

### Step k

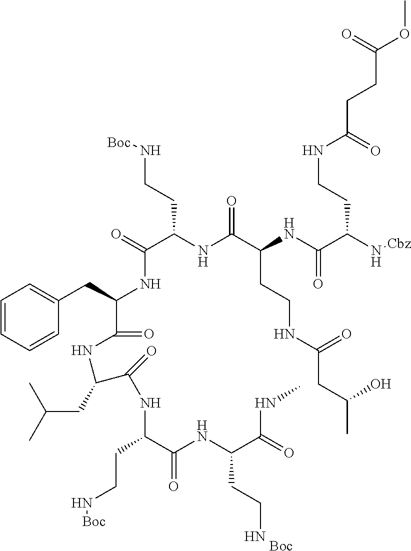

To a 0° C. stirring solution of product from step J (0.215 g, 0.166 mmol), monomethyl succinate (0.022 g, 0.166 mmol), and DIEA (0.072 mL, 0.414 mmol) in DMF (5.0 mL), it was added HATU (0.066 g, 0.174 mmol) in DMF (2.0 mL), dropwise. Temperature was slowly allowed to reach ambient temperature over 15 min. Upon completion, all the volatiles were removed by rotary evaporation. The residue was purified by RPLC using an Isco CombiFlash liquid chromatograph eluted with 50% to 100% water and methanol, using no modifier. Yield 0.201 g, 86% yield.

### Step l

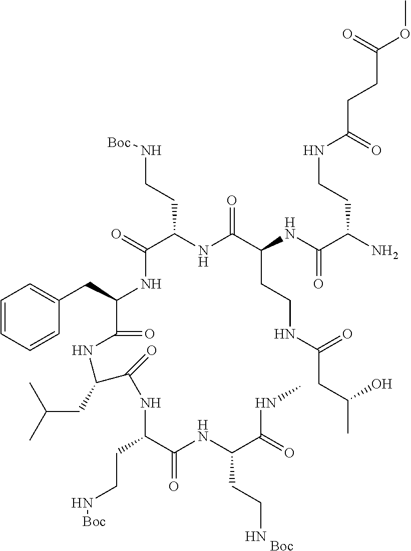

Product from step k (0.201 g, 0.143 mmol) was dissolved in methanol (10 mL) and suspended with SiliaCatPd(0)® (0.071 g, 0.014 mmol) under hydrogen atmosphere (˜1 atm). When all the starting material was consumed by HPLC analysis, the mixture was filtered over a short Celite pad, and all the volatiles were removed by rotary evaporation. HPLC analysis of the residue revealed a single peak, which was used in the next step without further purification. Yield 1.075 g, 99% yield. ¹H NMR for the diagnostic peaks: ¹H NMR (Methanol-d₄) δ: 7.37-7.20 (m, 5H), 3.67 (s, 3H), 2.63 (t, J=6.7 Hz, 2H), 2.49 (t, J=6.7 Hz, 1H), 1.44 (br s, 27H), 1.20 (d, J=6.4 Hz, 3H), 0.67 (br d, J=18.1 Hz, 6H).

### Step m

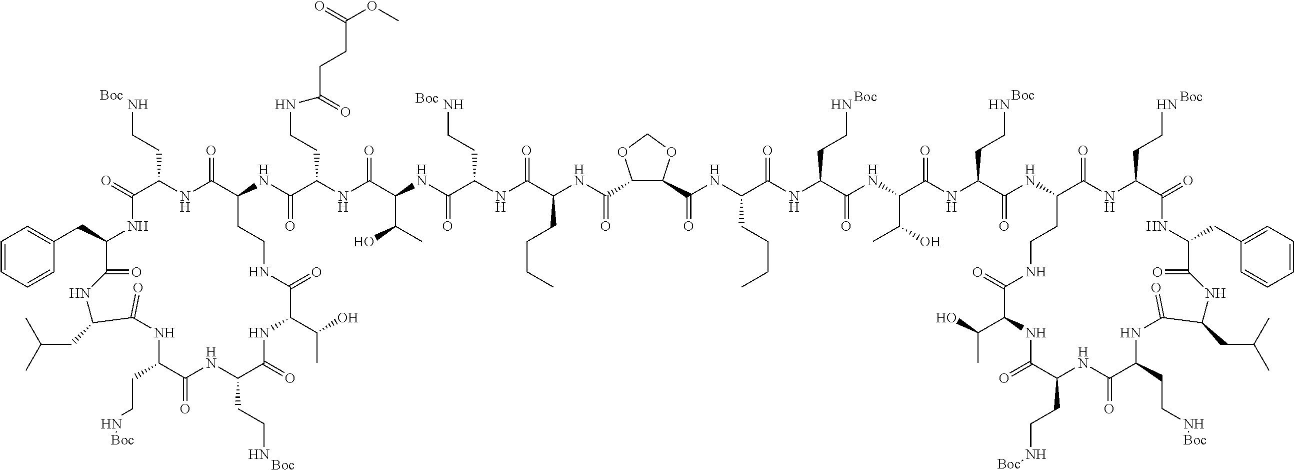

To a 0° C. stirring solution of product from step 1 (0.1 mmol), product from step h (0.1 mmol), and DIEA (0.2 mmol) in DMF (5.0 mL), is added HATU (0.1 mmol) in DMF (1.5 mL), dropwise. Temperature is slowly allowed to reach ambient temperature over 15 min. Upon completion, all the volatiles are evaporated by rotary evaporation. The residue is purified by RPLC using an Isco CombiFlash liquid chromatograph eluted with 50% to 100% water and methanol, using no modifier.

### Step n

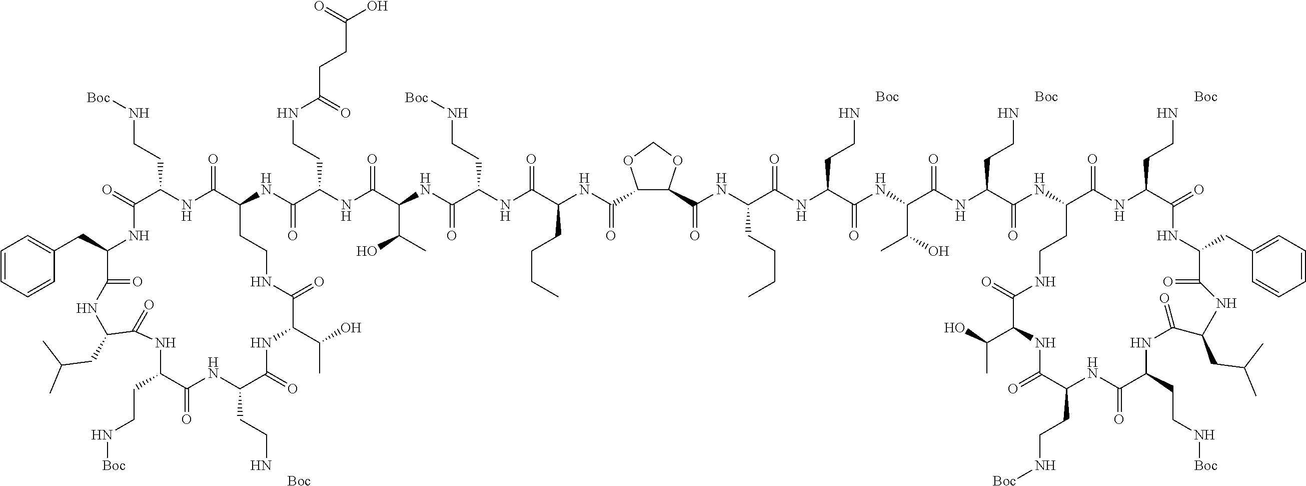

Product from step m (0.1 mmol) is taken up in 1,2-dichloroethane (5.0 mL), treated with trimethyltin hydroxide (1.0 mmol), and the mixture is refluxed until completion. Upon cooling, the mixture is treated under stirring with SiliaMetS® (2.0 mmol) for 1 h. All the volatiles are evaporated by rotary evaporation. The residue is taken up in acetic acid (5 mL) and filtered over a short celite pad. All the volatiles are evaporated by rotary evaporation, to afford the desired product.

### Step o

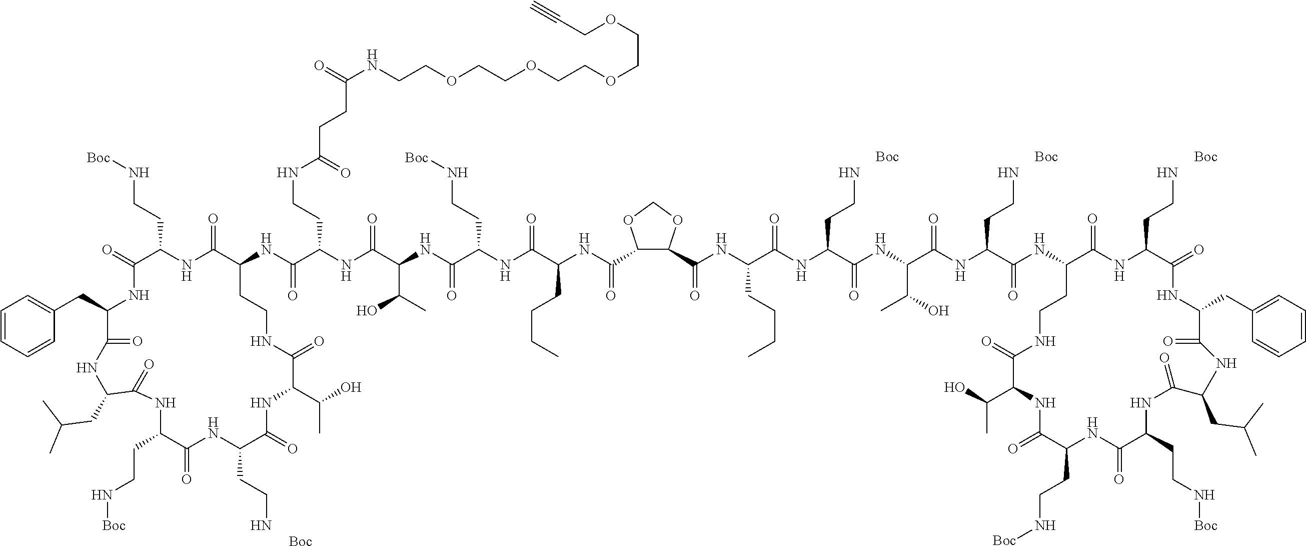

To a 0° C. stirring solution of product from step n (0.1 mmol), propargyl-PEG4-amine (0.1 mmol), and DIEA (0.2 mmol) in DMF (5.0 mL), is added HATU (0.1 mmol) in DMF (1.5 mL), dropwise. Temperature is slowly allowed to reach ambient temperature over 15 min. Upon completion, all the volatiles are evaporated by rotary evaporation. The residue is purified by RPLC using an Isco CombiFlash liquid chromatograph eluted with 50% to 100% water and methanol, using no modifier.

### Step p

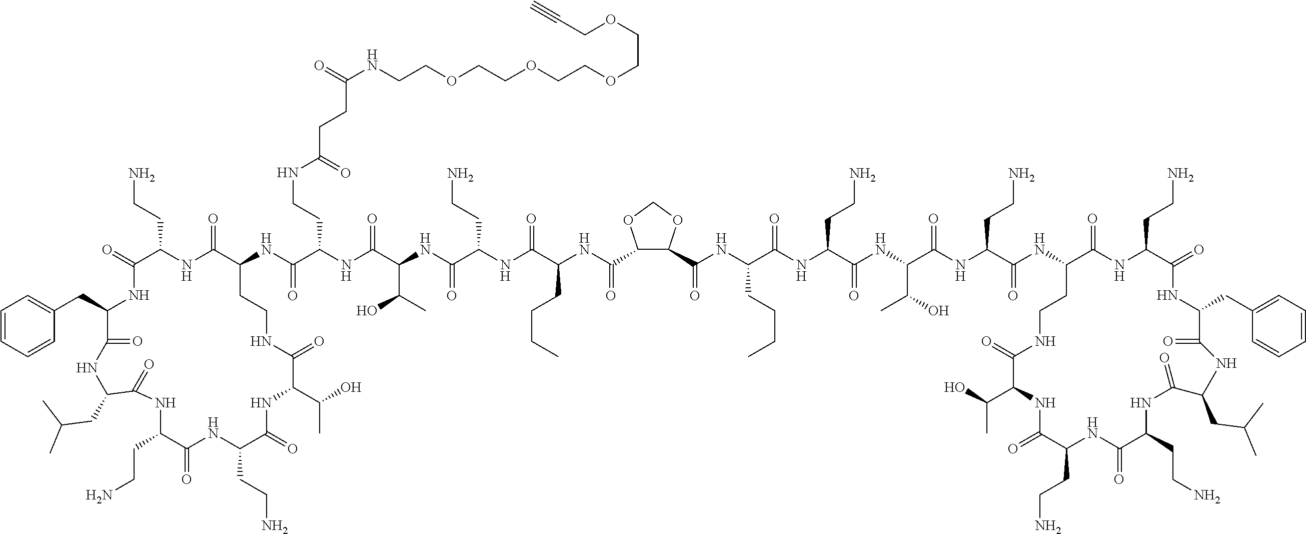 [SMILES: C#CCOCCOCCOCCOCCNC(=O)CCC(=O)NCC[C@H](NC(=O)[C@@H](NC(=O)[C@H](CCN)NC(=O)[C@H](CCCC)NC(=O)[C@@H]1OCO[C@H]1C(=O)N[C@@H](CCCC)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O)[C@@H](C)O)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O]

Product from step o (0.1 mmol) is treated with dichloromethane (4.0 mL), 2-methyl-2-butene (0.25 mL), and TFA (2.0 mL) until ceasing of gas evolution (bubbler monitoring), and then concentrated to an oil. The oil is purified by RPLC using an Isco CombiFlash liquid chromatograph eluted with 10% to 100% water and methanol, using 0.1% TFA as the modifier.

### Example 176—Synthesis of an AA3 Linked ADC

To a room temperature solution of product from Example 109, step p (0.056 mg, 10.2 μmol) in pH 7.4 PBS (500 μL), is added: a 20 mM solution of THPTA in pH 7.4 PBS (340 μL, 6.8 μmol), a 20 mM solution of sodium ascorbate in pH 7.4 PBS (680 μL, 13.6 μmol), and a 20 mM solution of copper sulfate in water (340 μL, 6.8 μmol). To this solution is added azido-hIgG1(Myc)Fc (0.020 mg, 0.340 μmol, 10 mg/mL, MW=58,833.00 Da, DAR=3) in pH 7.4 PBS. The resulting solution is agitated on a rocker table overnight at room temperature, then purified by affinity chromatography over a protein-A column (See Example 164).

### Example 177—Preparation of Extended Threonines (Int-77)

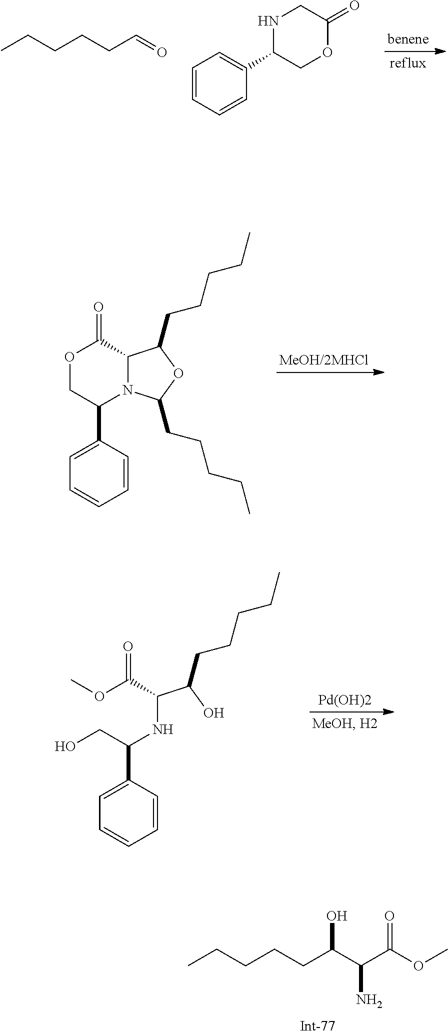

Extended threonine analogs were prepared as described in Aiker et al., Tetrahedron. 54:6089-6098 (1998).

### Example 178—Synthesis of Int-78

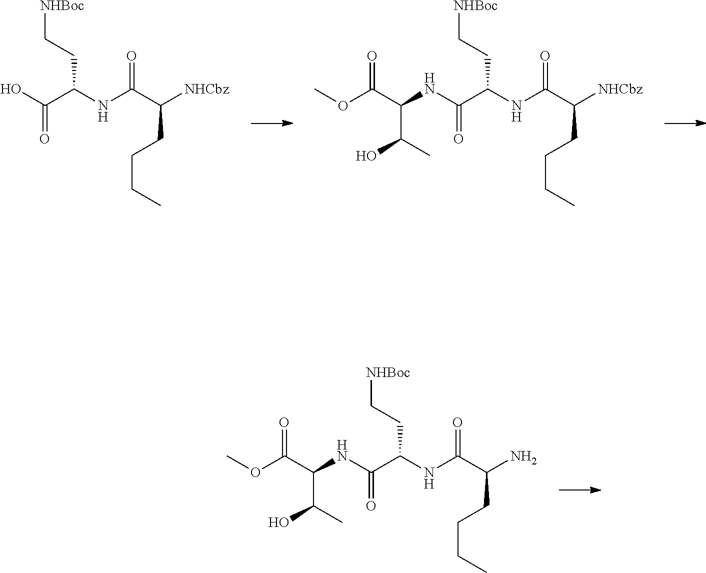 [SMILES: CCCC[C@H](NC(=O)OCc1ccccc1)C(=O)N[C@@H](CCNC(=O)OC(C)(C)C)C(=O)O ; CCCC[C@H](NC(=O)OCc1ccccc1)C(=O)N[C@@H](CCNC(=O)OC(C)(C)C)C(=O)N[C@H](C(=O)OC)[C@@H](C)O ; CCCC[C@H](N)C(=O)N[C@@H](CCNC(=O)OC(C)(C)C)C(=O)N[C@H](C(=O)OC)[C@@H](C)O]

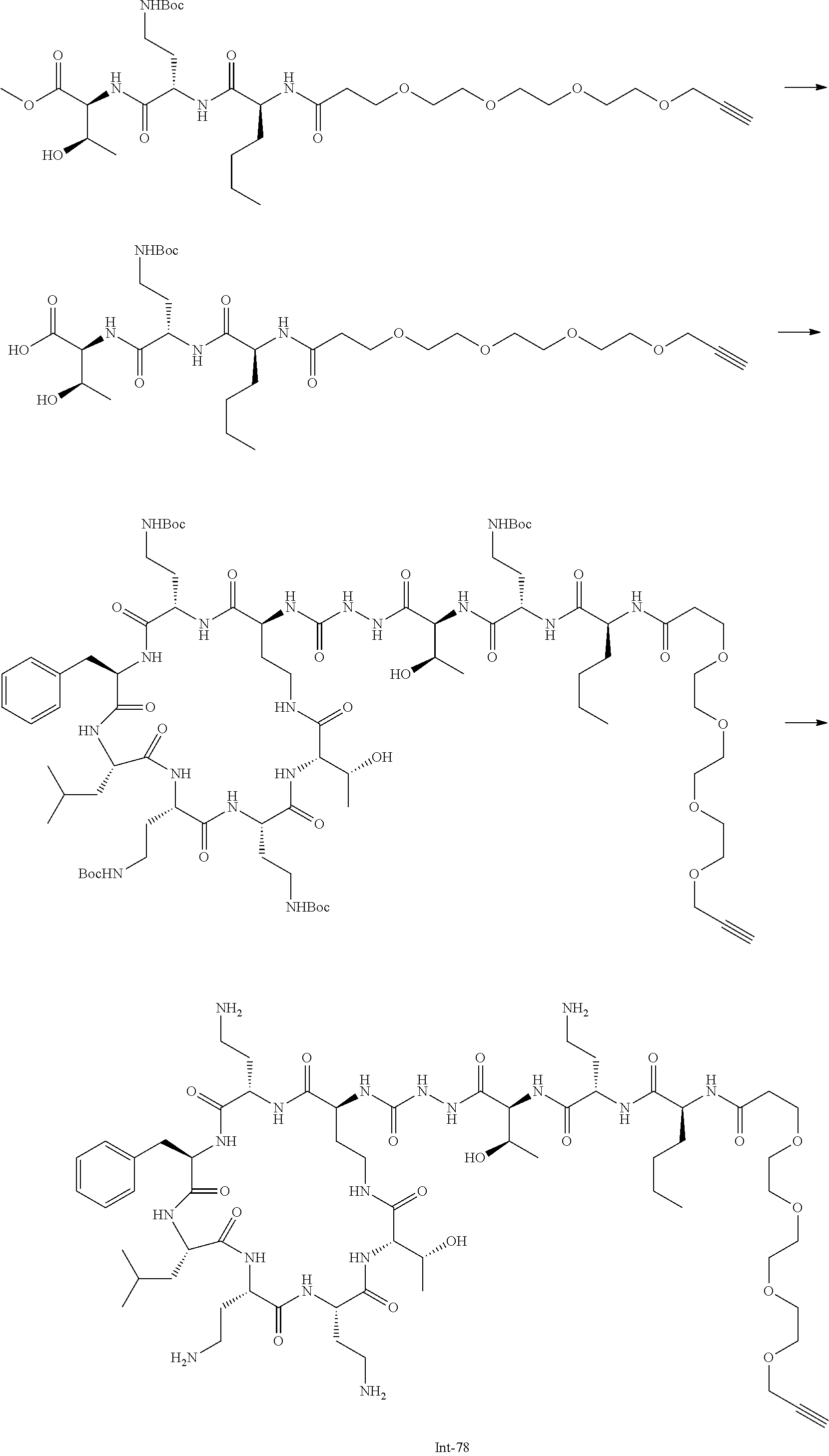

### Step a

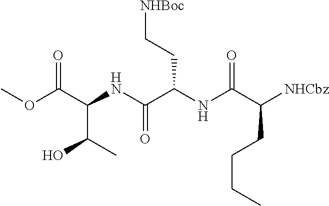 [SMILES: CCCC[C@H](NC(=O)OCc1ccccc1)C(=O)N[C@@H](CCNC(=O)OC(C)(C)C)C(=O)N[C@H](C(=O)OC)[C@@H](C)O]

To a solution of a mixture of Z-Nleu-Dab-OH (371 mg, 0.792 mmol) and L-threonine methyl ester HCl (169.6 mg, 1 mmol) in anhydrous DMF (2 ml) was added HATU (304 mg, 0.8 mmol). After stirring to dissolve HATU, the reaction mixture was treated with DIPEA (390 mg, 3 mmol) and stirred for 30 more minutes. It was then directly purified by RPLC (100 g column, 20 to 75% acetonitrile and water, using 0.1% TFA as modifier). Yield 451 mg, 98.1%. Ions found by LCMS: [M+H]⁺=581.2, [M-Boc+H]⁺=481.2.

### Step b

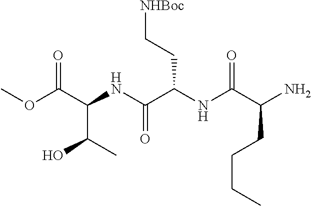 [SMILES: CCCC[C@H](N)C(=O)N[C@@H](CCNC(=O)OC(C)(C)C)C(=O)N[C@H](C(=O)OC)[C@@H](C)O]

The step a product (451 mg, 0.777 mmol) was dissolved in MeOH (20 ml). The solution was treated with Pd/C and stirred under hydrogen for 4 hours. Pd/C was removed by filtration. The filtrate was concentrated and purified by RPLC (100 g column, 5 to 60% acetonitrile and water, using 0.1% TFA as modifier). Yield 284 mg, 81.9%. Ion found by LCMS: [M+H]⁺=447.2.

### Step c

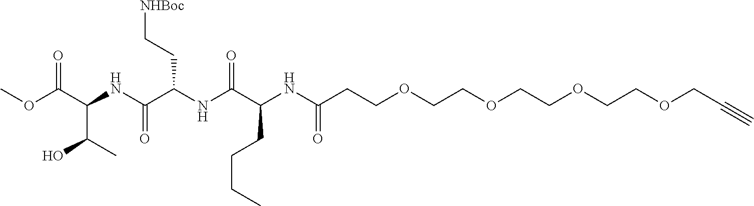 [SMILES: C#CCOCCOCCOCCOCCC(=O)N[C@@H](CCCC)C(=O)N[C@@H](CCNC(=O)OC(C)(C)C)C(=O)N[C@H](C(=O)OC)[C@@H](C)O]

To a solution of a mixture of propargyl-PEG4-acid (234.3 mg, 0.9 mmol) and HATU (353.6 mg, 0.93 mmol) in anhydrous DMF (1.5 ml) was added DIPEA (260 mg, 2 mmol). After the resulting mixture was stirred for 10 minutes, it was treated with the step b product (284 mg, 0.636 mmol) and continued stirring for 30 minutes. The reaction mixture was purified by RPLC (100 g column, 5 to 65% acetonitrile and water). Yield 220.5 mg, 50.3%. Ions found by LCMS: [M+H]⁺=689.2, [M-Boc+H]⁺=589.2.

### Step d

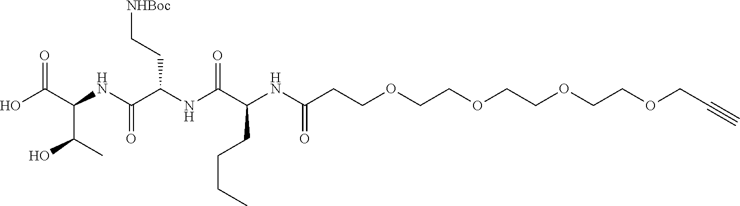 [SMILES: C#CCOCCOCCOCCOCCC(=O)N[C@@H](CCCC)C(=O)N[C@@H](CCNC(=O)OC(C)(C)C)C(=O)N[C@H](C(=O)O)[C@@H](C)O]

The step c product (220.5 mg, 0.32 mmol) in MeOH (1 ml) was cooled in ice-water bath and treated with a solution of LiOH monohydrate (25.2 mg, 0.6 mmol) in water (1 ml) in portions over 30 minutes. After stirring for 1 hour, the reaction mixture was acidified with 4N HCl solution and purified by RPLC (5 to 70% acetonitrile and water). Yield 165.5 mg, 76.6%. Ions found by LCMS: [M+H]⁺=675.3. [M-Boc+H]⁺=575.3.

### Step e

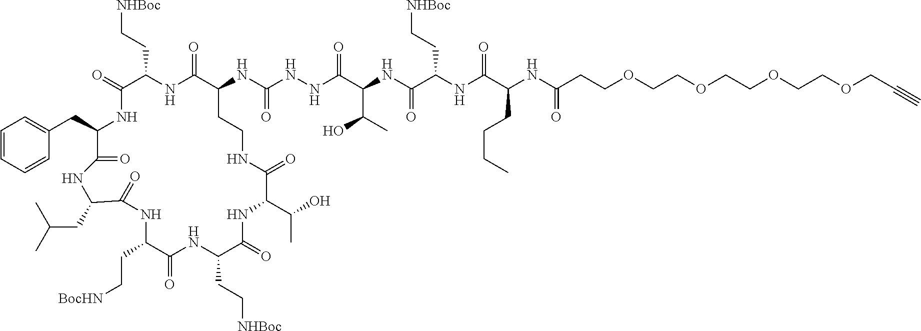 [SMILES: C#CCOCCOCCOCCOCCC(=O)N[C@@H](CCCC)C(=O)N[C@@H](CCNC(=O)OC(C)(C)C)C(=O)N[C@H](C(=O)NNC(=O)N[C@H]1CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](Cc2ccccc2)NC(=O)[C@H](CCNC(=O)OC(C)(C)C)NC1=O)[C@@H](C)O]

A mixture of the step d product (101.2 mg, 0.15 mmol) and HATU (57.0 mg, 0.153 mmol) was dissolved in anhydrous DMF (1 ml) and DIPEA (39 mg, 0.3 mmol). After stirring for 10 minutes, the reaction mixture was treated with PMBH-AzaGly-NH2 (168 mg, 0.15 mmol, described in step b of Example 192) and continued stirring for 1 hour. It was then directly purified by RPLC (30 to 90% MeOH and water, using 0.1% TFA as modifier). Yield 233.6 mg, 87.7%. Ion found by LCMS: [(M−2Boc+2H)/2]⁺=789.

### Step f

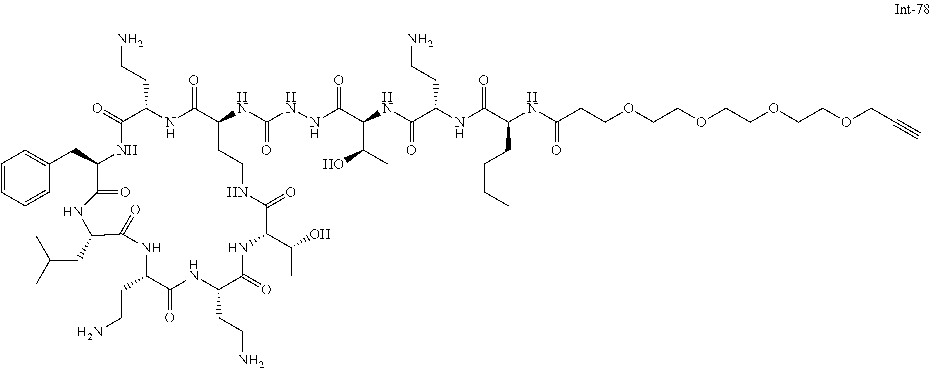 [SMILES: C#CCOCCOCCOCCOCCC(=O)N[C@@H](CCCC)C(=O)N[C@@H](CCN)C(=O)N[C@H](C(=O)NNC(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O]

The step e product (233.6 mg, 0.131 mmol) was dissolved in TFA (0.3 ml). The reaction was stirred for 20 minutes, then concentrated and purified by RPLC (50 g, 5 to 35% acetonitrile and water, using 0.1% TFA as modifier). Yield 199.6 mg, 83.1%. Ions found by LCMS: [(M+2H)/2]⁺=689, [(M+3H)/3]⁺=459.8, [(M+4H)/4]⁺=345.0.

### Example 179—Synthesis of Int-79

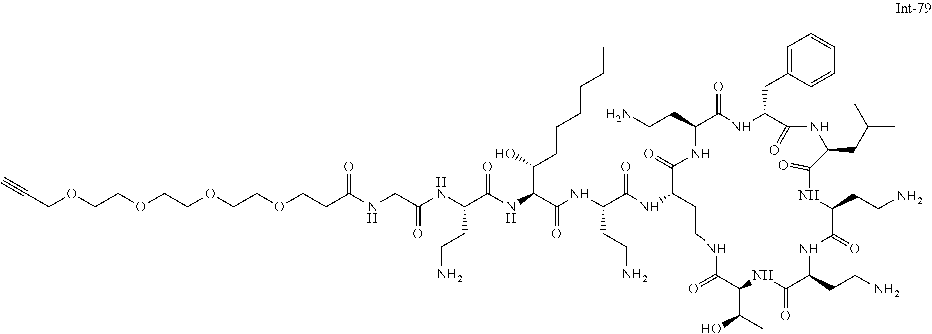 [SMILES: C#CCOCCOCCOCCOCCC(=O)NCC(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCCCCC]

The title compound was prepared analogously to Example 194, where the (S)-methyl 2-aminohexanoate was substituted with methyl glycinate in the step a. Ion(s) found by LCMS: [(M+2H)/2]+=716.9, [(M+3H)/3]+=478.3, [(M+4H)/4]+=358.9.

### Example 180—Synthesis of Int-80

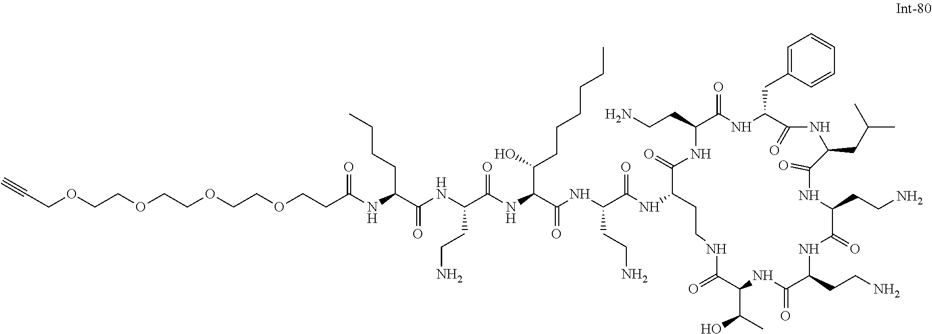 [SMILES: C#CCOCCOCCOCCOCCC(=O)N[C@@H](CCCC)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCCCCC]

The title compound was prepared analogously to Example 194, where the hexanal was substituted for heptanal in the preparation of the extended threonine (see Example 177). Ion(s) found by LCMS: [(M+2H)/2]⁺=744.9, [(M+3H)/3]⁺=497.0, [(M+4H)/4]⁺=373.0.

### Example 181—Synthesis of Int-81

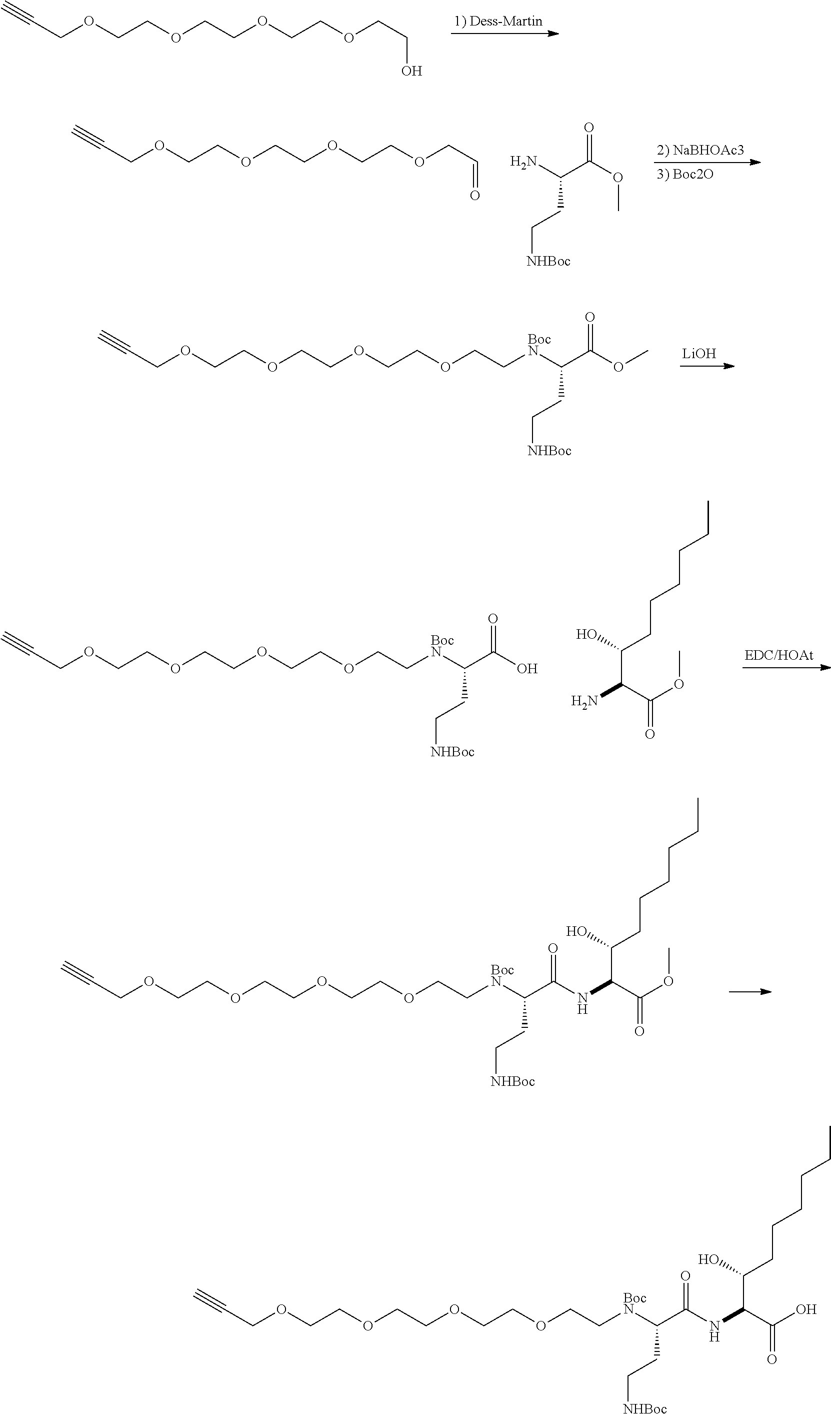

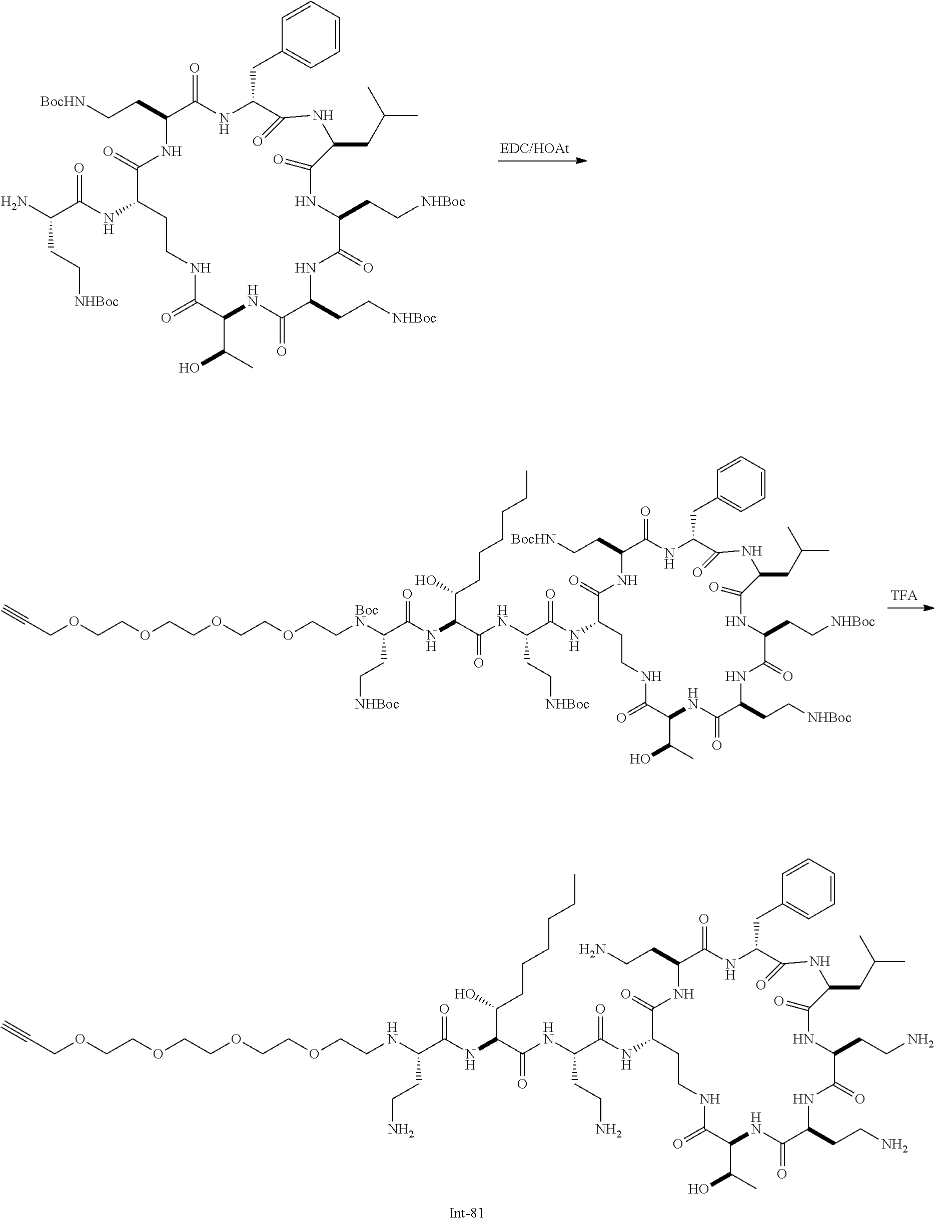

### Step a

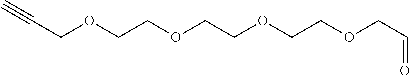 [SMILES: C#CCOCCOCCOCCOCC=O]

Propargyl-PEG4-alcohol (1.48 g, 6.4 mmol) was dissolved in 50 ml DCM and cooled with an ice-water bath. To this solution was added Dess Martin periodinane (3.25 g, 1.2 eq) in small portions over 5 minutes. The resulting solution was stirred for 2 hrs, then filtered. The filtrate was used for next step without any workup or purification.

### Step b

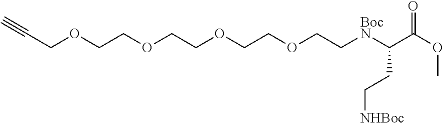 [SMILES: C#CCOCCOCCOCCOCCN(C(=O)OC(C)(C)C)[C@@H](CCNC(=O)OC(C)(C)C)C(=O)OC]

L-Dab(Boc)-OMe (0.76 g, 3.3 mmol) was added to a solution of propargyl-PEG4-aldehyde in DCM (0.63 g, 2.7 mmol). Sodium triacetoxyborohydride (2.5 g, 1.1 equi.) was added above solution in small portions over 5 minutes. The solution was stirred overnight, and then Boc₂O (0.8 g, 4 mmol) and triethylamine (1.4 ml, 10 mmol) were added. The resulting reaction was stirred for 2 his. The reaction was concentrated and purified by reverse phase liquid chromatography (RPLC) using an Isco CombiFlash liquid chromatograph eluted with 0% to 100% acetonitrile and water, using TFA acid as the modifier. Yield of 1.20 g as TFA salt, 61% yield. Ion(s) found by LCMS: [M]+1=547.3, [M-Boc]+1=447.3.

### Step c

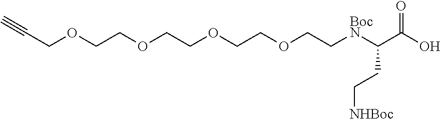 [SMILES: C#CCOCCOCCOCCOCCN(C(=O)OC(C)(C)C)[C@@H](CCNC(=O)OC(C)(C)C)C(=O)O]

Lithium hydroxide (60 mg, 2.5 mmol) in 2 ml water was added into the solution of the amino acid ester from step b (0.50 g, 1 mmol) in 2 ml MeOH and 2 ml THF, LCMS monitored the progress of the reaction. After completion, the solution was acidified with Amberlite® IRN-77, ion exchange resin to pH 2, then the resulted solution was filtered and the filtrate was concentrated and used for next step without purification. Ion(s) found by LCMS: by LCMS: [M]+1=533.3, [M-Boc]+1=433.3.

### Step d

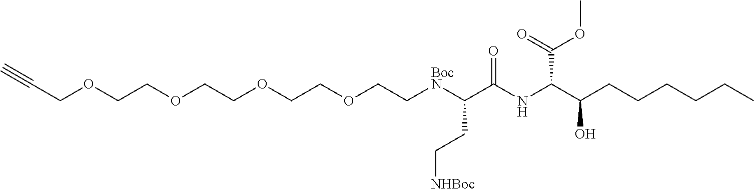 [SMILES: C#CCOCCOCCOCCOCCN(C(=O)OC(C)(C)C)[C@@H](CCNC(=O)OC(C)(C)C)C(=O)N[C@H](C(=O)OC)[C@H](O)CCCCCC]

Product from the previous step (230 mg, 0.43 mmol) and methyl (2S,3R)-2-amino-3-hydroxynonanoate (90 mg, 0.44 mmol) in DMF (30 ml) was added EDC (1.2 g, 6 mmol), HOBT (0.8 g, 6 mmol), and DIPEA (1.4 ml, 10 mmol) at room temperature. The solution was stirred overnight. The resulted solution was concentrated and purified by reverse phase liquid chromatography (RPLC) using an Isco CombiFlash liquid chromatograph eluted with 5% to 100% acetonitrile and water, using 0.1% TFA as the modifier. Ion(s) found by LCMS: (M)+1=718.4. (M−Boc)+1=618.4

### Step e

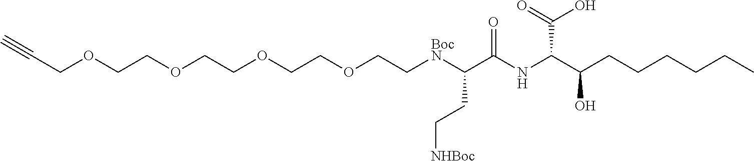 [SMILES: C#CCOCCOCCOCCOCCN(C(=O)OC(C)(C)C)[C@@H](CCNC(=O)OC(C)(C)C)C(=O)N[C@H](C(=O)O)[C@H](O)CCCCCC]

Lithium hydroxide (24 mg, 1 mmol) in 2 ml water was added to a solution of amino acid ester from step d (0.120 g, 0.15 mmol) in 2 ml MeOH and 2 ml THF. Progress of the reaction was monitored by LCMS. After the completion, the reaction was acidified with Amberlite® IRN-77 ion exchange resin to pH 2 then filtered. The filtrate was concentrated and used in next step without purification. Ion(s) found by LCMS: by LCMS: [M]+1=714.4, [M-Boc]+1=604.4.

### Step f

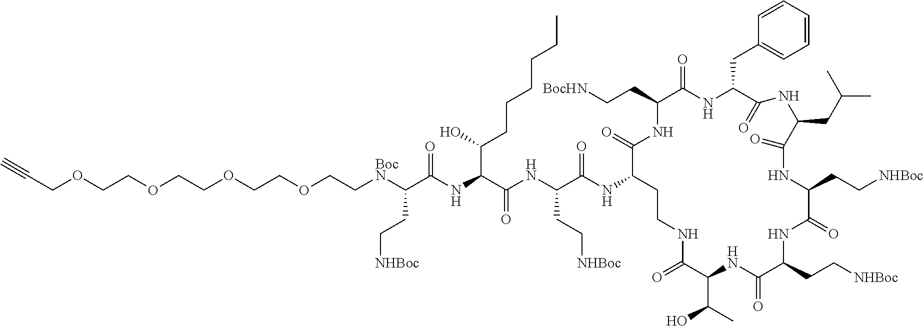 [SMILES: C#CCOCCOCCOCCOCCN(C(=O)OC(C)(C)C)[C@@H](CCNC(=O)OC(C)(C)C)C(=O)N[C@H](C(=O)N[C@@H](CCNC(=O)OC(C)(C)C)C(=O)N[C@H]1CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](Cc2ccccc2)NC(=O)[C@H](CCNC(=O)OC(C)(C)C)NC1=O)[C@H](O)CCCCCC]

Acid from the previous step (100 mg, 0.14 mmol), and tetra-Boc-polymyxin B octapeptide (190 mg, 0.15 mmol) in DMF (10 ml) were added EDC (100 mg, 0.5 mmol), HOBT (50 mg, 0.3 mmol), and DIEA (0.14 ml, 1.0 mmol) at room temperature. The solution was stirred overnight. The resulting solution was concentrated and re-dissolved into 2 ml methanol. The resulting solution was added dropwise into 50 ml vigorously stirred water, this heterogeneous solution was stirred for 1 hour, then filtered with Buchner funnel with filter paper. The crude product was dried and used in next step without further purification.

### Step g

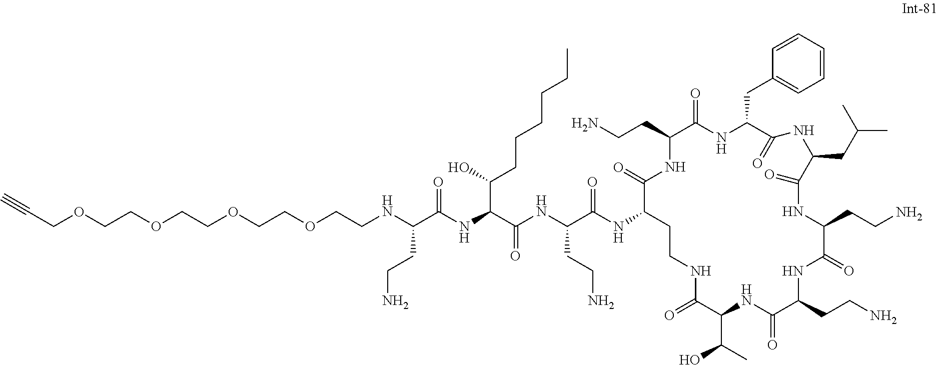 [SMILES: C#CCOCCOCCOCCOCCN[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCCCCC]

Product from step f was treated in 2 ml DCM and 2 ml TFA at room temperature. The solution was stirred 10 min, then concentrated and purified by reverse phase liquid chromatography (RPLC) using an Isco CombiFlash liquid chromatograph eluted with 0% to 100% acetonitrile and water, using TFA as the modifier. Yield of 170 mg as TFA salt, 61% yield. Ion(s) found by LCMS: [(M+2H)/2]+=674.4, [(M+3H)/3]⁺=449.9, [(M+4H)/4]⁺=337.7.

### Example 162—Synthesis of Int-82

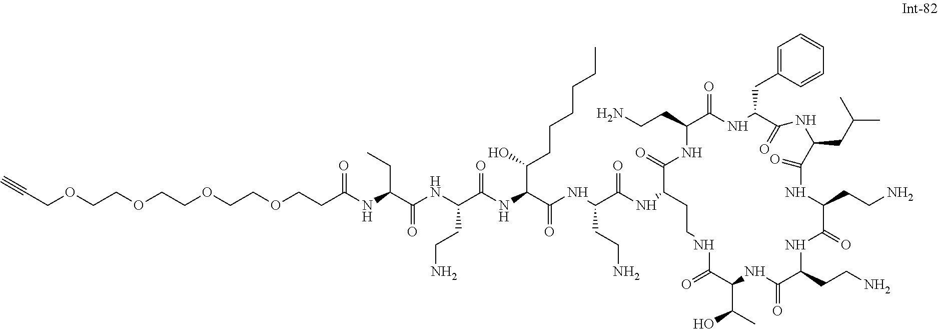 [SMILES: C#CCOCCOCCOCCOCCC(=O)N[C@@H](CC)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCCCCC]

The title compound was prepared analogously to Example 194. Ion(s) found by LCMS: [(M+2H)/2]⁺=730.4, [(M+3H)/3]⁺=487.9, [(M+4H)/4]⁺=386.2.

### Example 183—Synthesis of Int-83

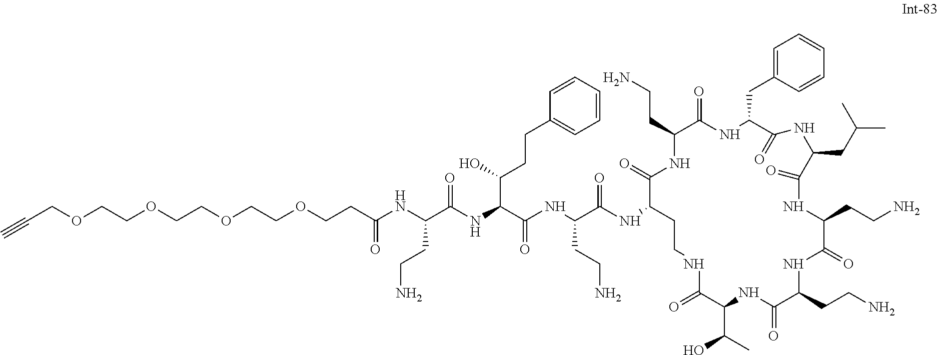 [SMILES: C#CCOCCOCCOCCOCCC(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCc1ccccc1]

The title compound was prepared analogously to Example 195, where the C4 extended threonine was replaced with benzyl extended threonine (prepared by replacing the hexanal with 3-phenylpropionaldehyde in the first step of Example 177). Ion(s) found by LCMS: [(M+2H)/2]⁺=698.8, [(M+3H)/3]⁺=466.2, [(M+4H)/4]⁺=349.9.

### Example 184—Synthesis of Int-84

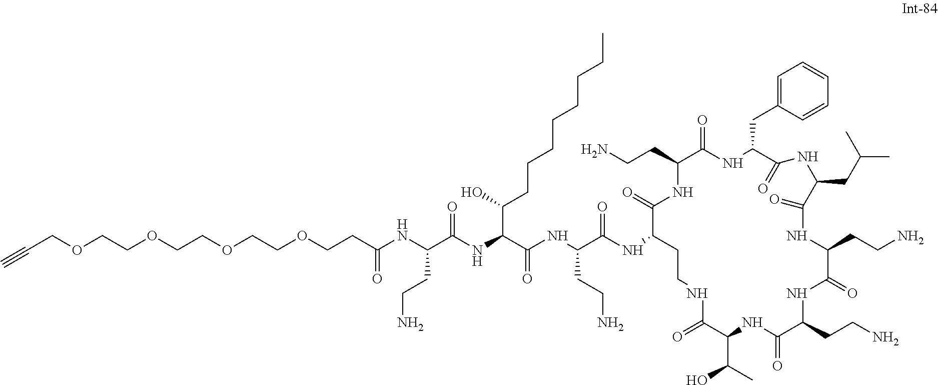 [SMILES: C#CCOCCOCCOCCOCCC(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCCCCCCC]

The title compound was prepared analogously to Example 195, where the C5 extended threonine was replaced with C7 extended threonine. Ion(s) found by LCMS: [(M+2H)/2]⁺=702.9, [(M+3H)/3]⁺=468.9, [(M+4H)/4]⁺=351.9.

### Example 185—Synthesis of Int-85

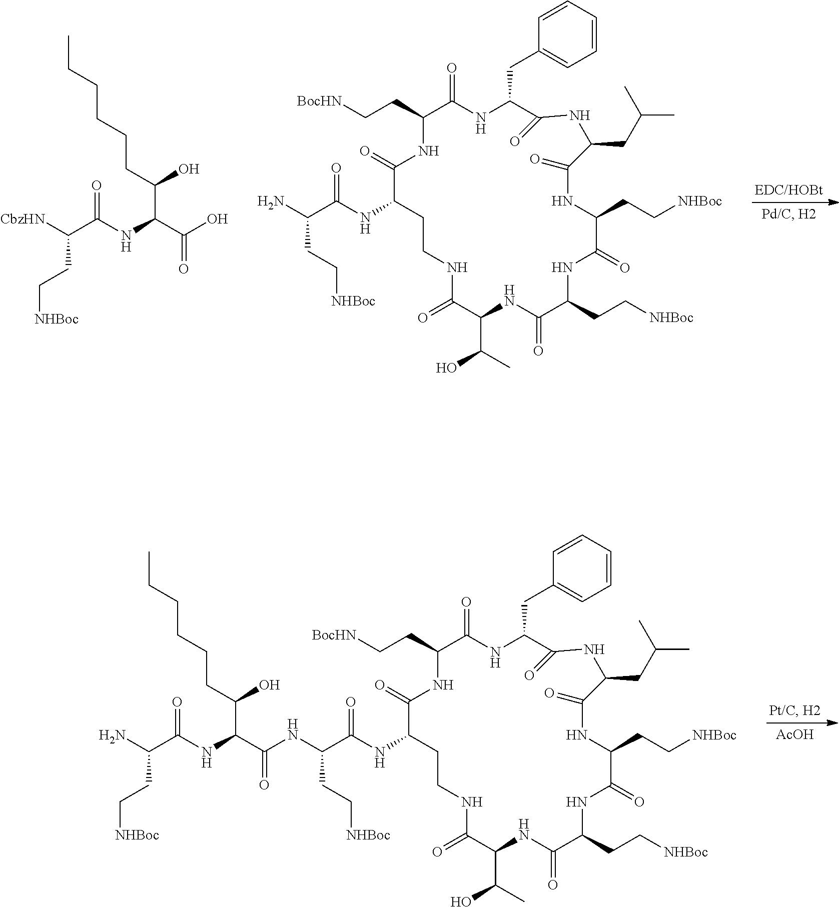

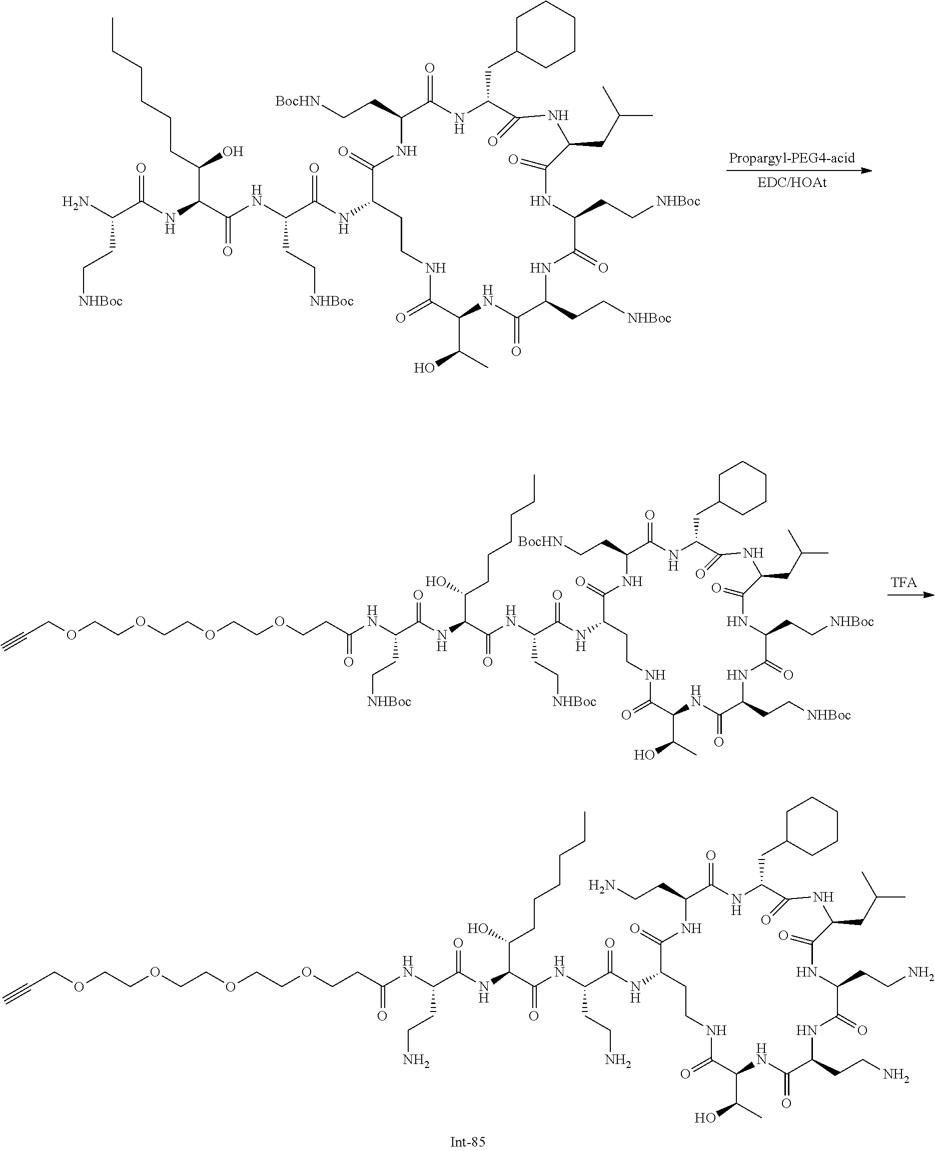

### Step a

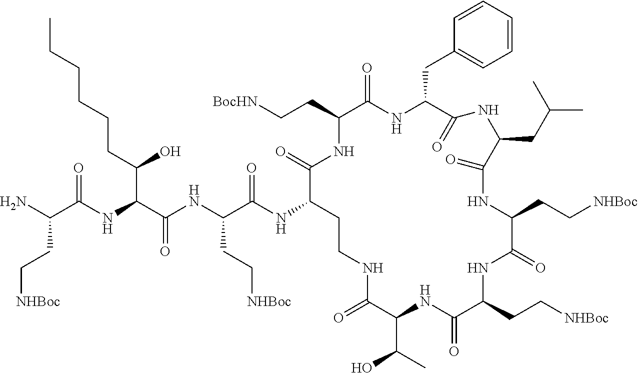 [SMILES: CCCCCC[C@@H](O)[C@H](NC(=O)[C@@H](N)CCNC(=O)OC(C)(C)C)C(=O)N[C@@H](CCNC(=O)OC(C)(C)C)C(=O)N[C@H]1CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](Cc2ccccc2)NC(=O)[C@H](CCNC(=O)OC(C)(C)C)NC1=O]

The title compound was prepared analogously to Example 194, where the C4 extended threonine component was replaced C5 extended threonine. Ion(s) found by LCMS: [(M+2H)/2]⁺=817.5, [(M+3H)/3]⁺=545.3, [(M+4H)/4]⁺=409.2.

### Step b

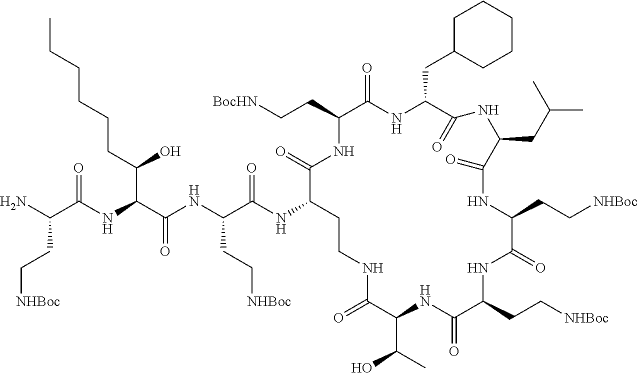 [SMILES: CCCCCC[C@@H](O)[C@H](NC(=O)[C@@H](N)CCNC(=O)OC(C)(C)C)C(=O)N[C@@H](CCNC(=O)OC(C)(C)C)C(=O)N[C@H]1CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](CC2CCCCC2)NC(=O)[C@H](CCNC(=O)OC(C)(C)C)NC1=O]

Penta-Boc polymyxin B decapeptide with C5-extended threonine (1 g, 0.61 mmol) was slowly added to acetic acid (5 ml). After stirring to dissolve all starting material, the solution was treated with PtO₂ (230 mg, 1 mmol) and stirred under hydrogen from a double layer of balloons for 24 h. The hydrogen was then flushed with nitrogen, then the PtO₂ was removed by filtering through celite. The filtrate was concentrated by rotary evaporation, and purified by RPLC (100 g, 30 to 70% acetonitrile and water). Yield 610 mg, 68.1%. Ions found by LCMS: [(M+2H)/2]⁺=820.5, [(M+3H)/3]⁺=547.3, [(M+4H)/4]⁺=410.7.

### Step c

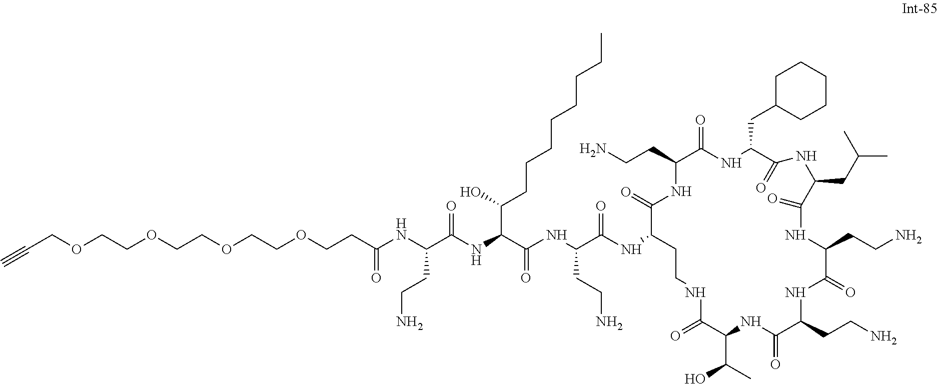 [SMILES: C#CCOCCOCCOCCOCCC(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](CC2CCCCC2)NC(=O)[C@H](CCN)NC1=O)[C@H](O)CCCCCCCC]

The title compound was prepared analogously to Example 194, where the acid intermediate (used in step g) was substituted with propargyl-PEG-4-acid. Ion(s) found by LCMS: [(M+2H)/2]+=691.8, [(M+3H)/3]⁺=461.6, [(M+4H)/4]⁺=346.4.

### Example 186—Synthesis of Int-86

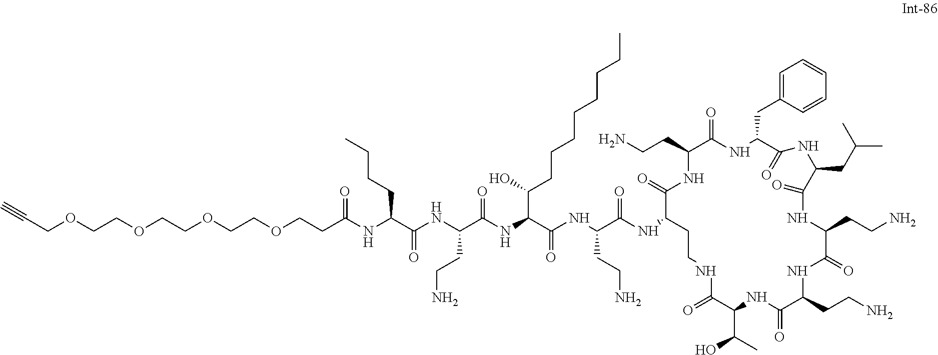 [SMILES: C#CCOCCOCCOCCOCCC(=O)N[C@@H](CCCC)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCCCCCCC]

The title compound was prepared analogously to Example 194, where the C4-extended threonine was substituted with C7-extended threonine. Synthesis of extended threonine derivatives is described in Example 177. Ion(s) found by LCMS: [(M+2H)/2]⁺=758.5, [(M+3H)/3]⁺=506.6, [(M+4H)/4]⁺=380.3.

### Example 187—Synthesis of Int-87

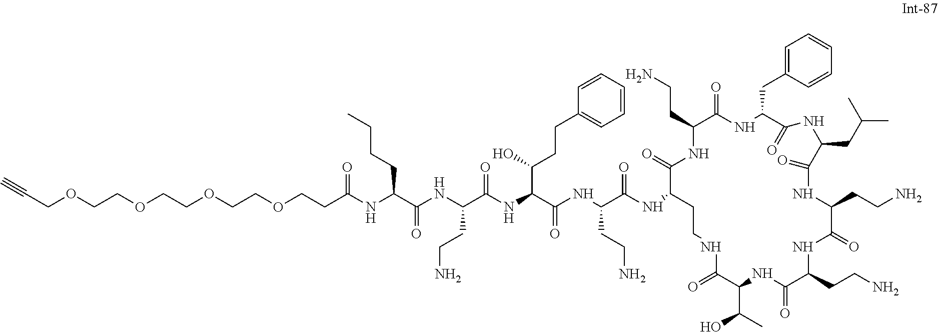 [SMILES: C#CCOCCOCCOCCOCCC(=O)N[C@@H](CCCC)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCc1ccccc1]

The title compound was prepared analogously to Example 194, where the C4-extended threonine was substituted with benzyl-extended threonine. Synthesis of extended threonine derivatives is described in Example 177. Ion(s) found by LCMS: [(M+2H)/2]⁺=754.9, [(M+3H)/3]⁺=503.6, [(M+4H)/4]⁺=377.9.

### Example 188—Synthesis of Int-88

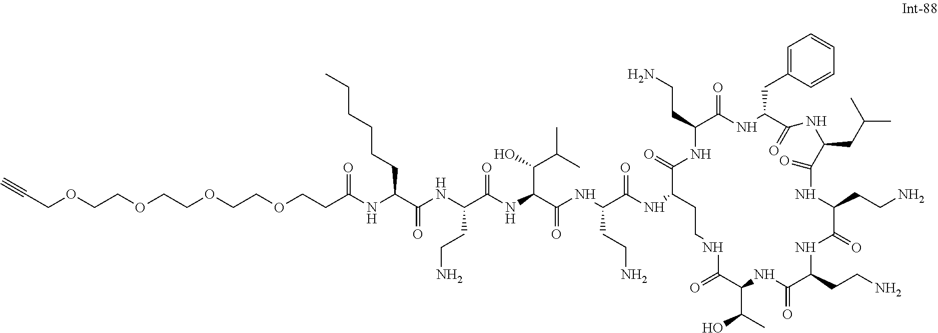 [SMILES: C#CCOCCOCCOCCOCCC(=O)N[C@@H](CCCCCC)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)C(C)C]

The title compound was prepared analogously to Example 194, where the C4-extended threonine was substituted with dimethyl-extended threonine. Synthesis of extended threonine derivatives is described in Example 177. Ion(s) found by LCMS: [(M+2H)/2]⁺=738.4, [(M+3H)/3]⁺=492.6, [(M+4H)/4]⁺=369.7.

### Example 189—Synthesis of Int-89

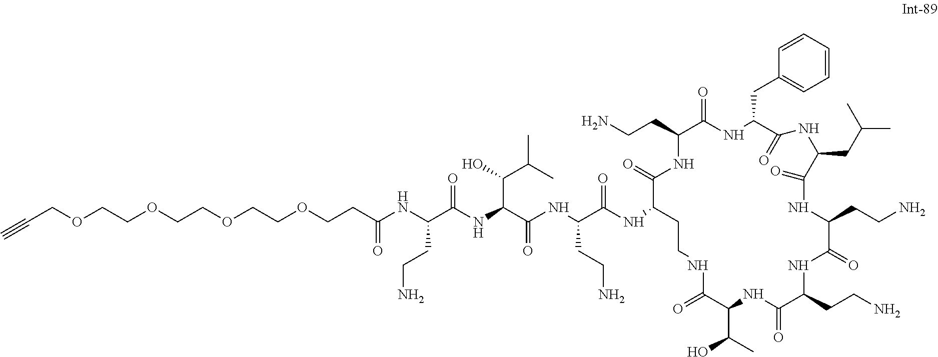 [SMILES: C#CCOCCOCCOCCOCCC(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)C(C)C]

The title compound was prepared analogously to Example 195 (C-1342), where the acid intermediate was substituted with propargyl-PEG-4-acid in the step g. Ion(s) found by LCMS: [(M+2H)/2]⁺=667.8, [(M+3H)/3]⁺=445.5, [(M+4H)/4]⁺=334.4.

### Example 190—Synthesis of Int-90

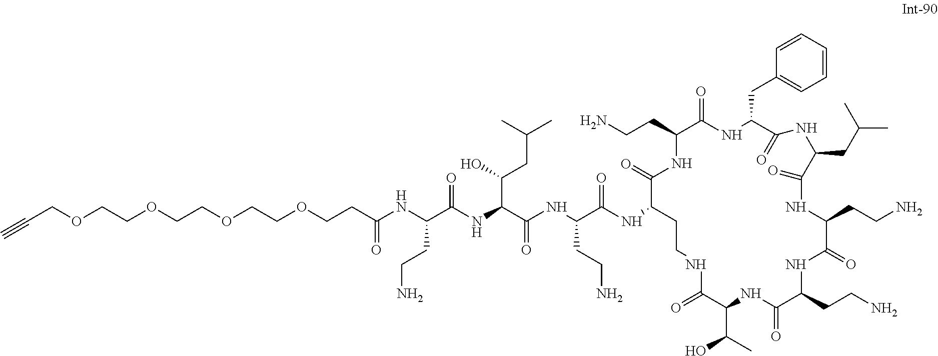 [SMILES: C#CCOCCOCCOCCOCCC(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CC(C)C]

The title compound was prepared analogously to Example 195, where the acid intermediate used in step g was replaced with propargyl-PEG-4-acid. Ion(s) found by LCMS: [(M+2H)/2]⁺=674.8, [(M+3H)/3]⁺=450.2, [(M+4H)/4]⁺=337.9

### Example 191—Synthesis of Int-91

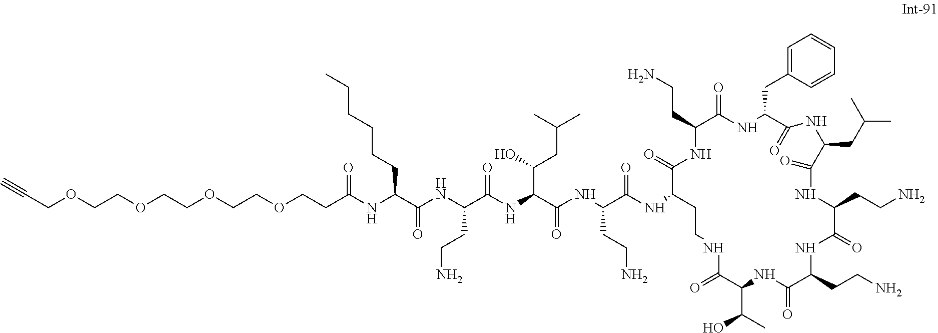 [SMILES: C#CCOCCOCCOCCOCCC(=O)N[C@@H](CCCCCC)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CC(C)C]

The title compound was prepared analogously to Example 194, where the C4-extended threonine was substituted with isopropyl-extended threonine. Synthesis of extended threonine derivatives is described in Example 177. Ion(s) found by LCMS: [(M+2H)/2]⁺=750.4, [(M+3H)/3]⁺=500.6, [(M+4H)/4]⁺=375.7.

### Example 192—Synthesis of Int-92

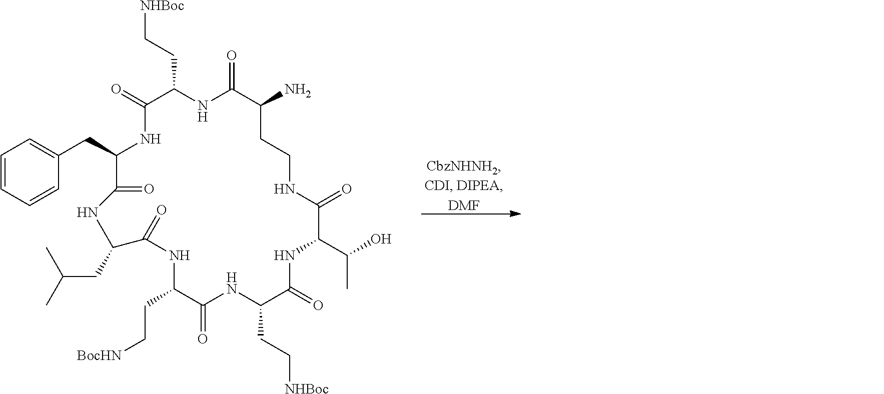 [SMILES: CC(C)C[C@@H]1NC(=O)[C@@H](Cc2ccccc2)NC(=O)[C@H](CCNC(=O)OC(C)(C)C)NC(=O)[C@@H](N)CCNC(=O)[C@H]([C@@H](C)O)NC(=O)[C@H](CCNC(=O)OC(C)(C)C)NC(=O)[C@H](CCNC(=O)OC(C)(C)C)NC1=O ; "CbzNHNH2,CDI, DIPEA,DMF"]

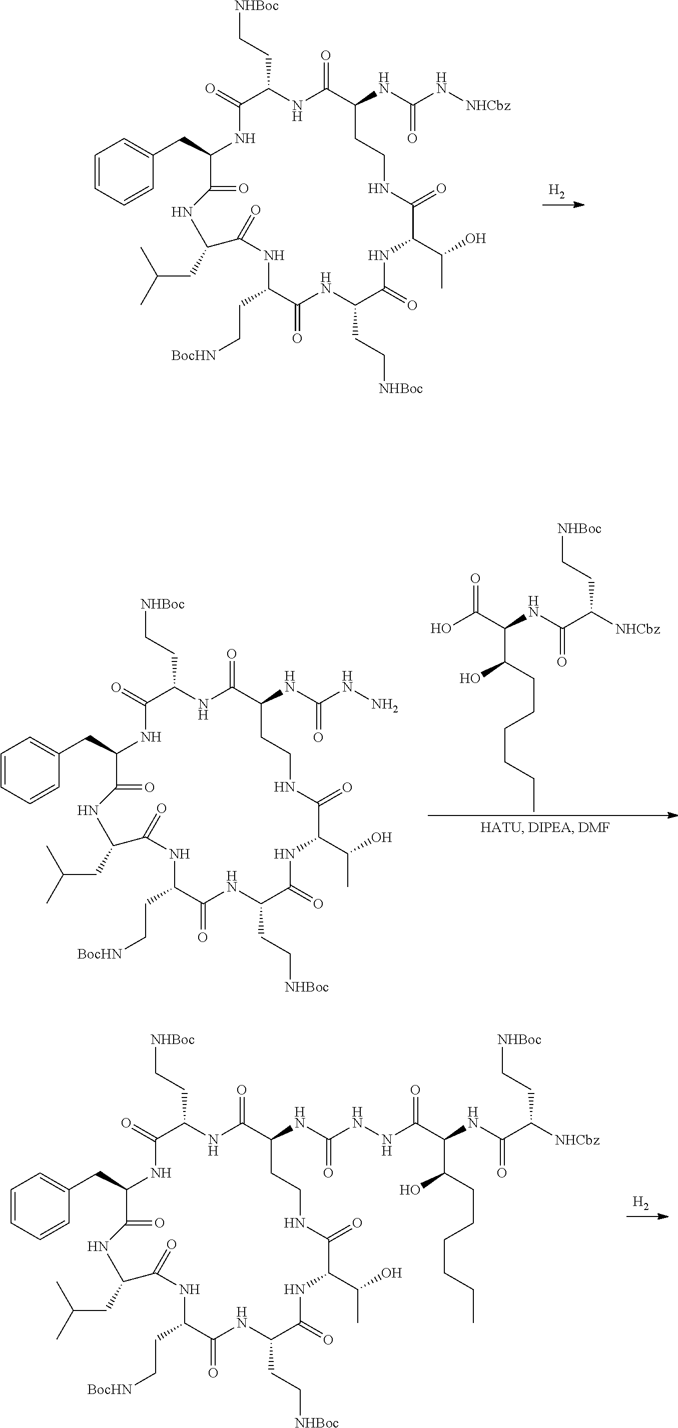

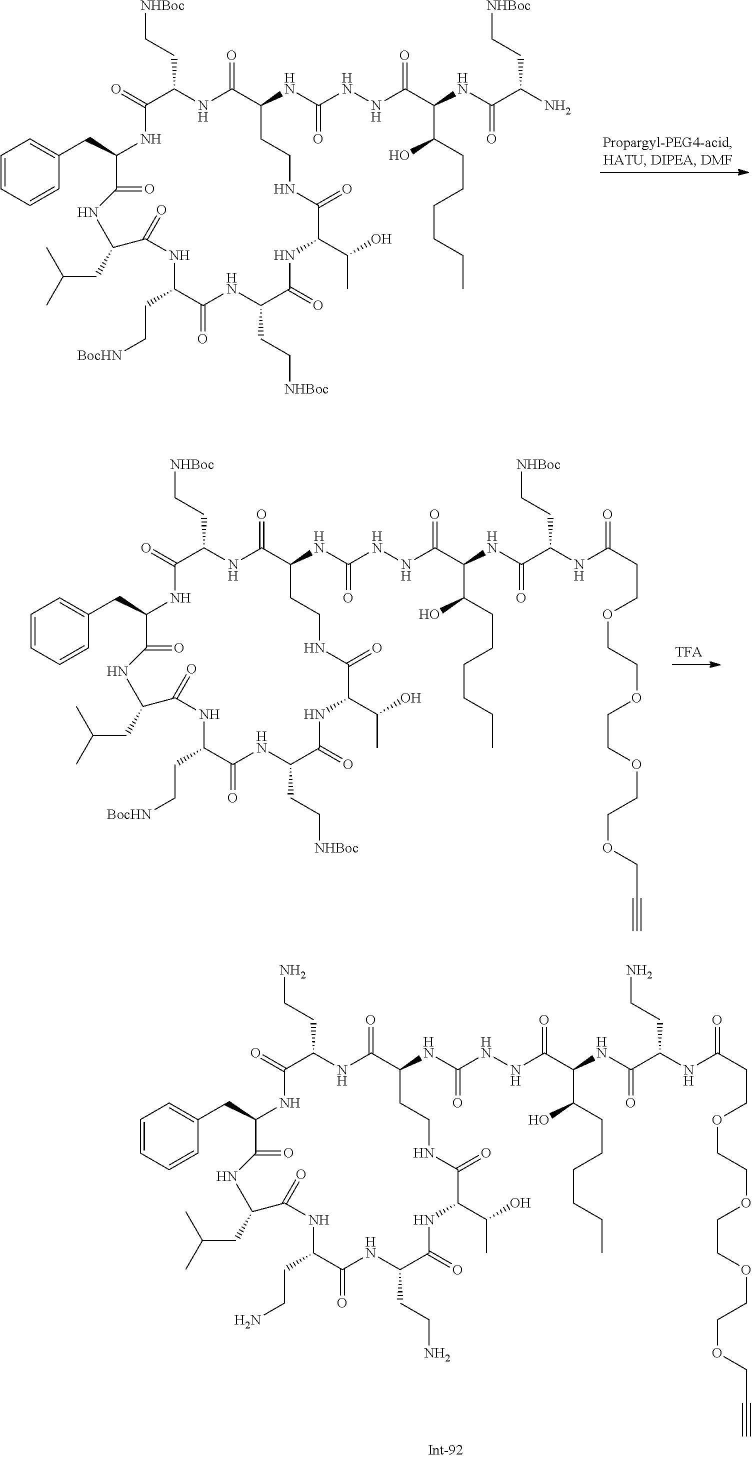

### Step a

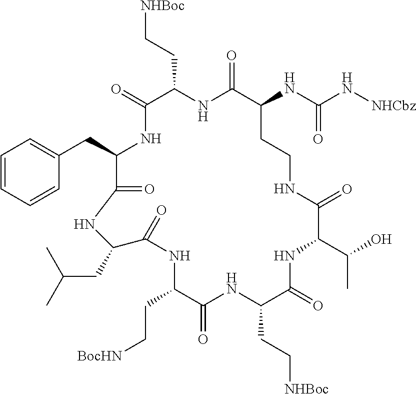 [SMILES: CC(C)C[C@@H]1NC(=O)[C@@H](Cc2ccccc2)NC(=O)[C@H](CCNC(=O)OC(C)(C)C)NC(=O)[C@@H](NC(=O)NNC(=O)OCc2ccccc2)CCNC(=O)[C@H]([C@@H](C)O)NC(=O)[C@H](CCNC(=O)OC(C)(C)C)NC(=O)[C@H](CCNC(=O)OC(C)(C)C)NC1=O]

A solution of 1,1′-carbonyldiimidazole (972.6 mg, 6 mmol) in anhydrous DMF (6 ml) was cooled in an ice-water bath and treated with benzyl carbazate (1.097 g, 6.6 mmol). After stirring to dissolve all the solids, the resulting mixture was treated with DIPEA (390 mg, 3 mmol). The ice-water bath was removed, and the reaction mixture was stirred for 1 hour. PMBH (2.126 g, 2 mmol, described in Example 2) was slowly added, and the reaction was continued overnight. The reaction mixture was then precipitated into water (100 ml) containing 4N HCl in dioxane (6 ml). The precipitated product was collected by vacuum filtration and washed with water. It was then re-dissolved in MeOH and concentrated to a white solid. The material was dissolved in DMF (2 ml) and purified by RPLC (150 g, 65 to 80% MeOH and water, using 0.1% TFA as modifier). Yield 1.32 g, 52.6%. Ions found by LCMS: [(M−2Boc+2H)/2]⁺=527.8, [(M−2Boc−tBu+2H)/2]⁺=499.8, [(M−3Boc+2H)/2]⁺=477.8.

### Step b

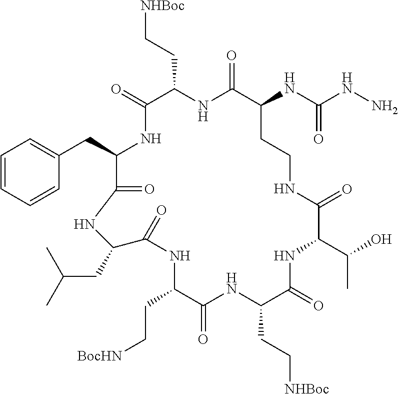 [SMILES: CC(C)C[C@@H]1NC(=O)[C@@H](Cc2ccccc2)NC(=O)[C@H](CCNC(=O)OC(C)(C)C)NC(=O)[C@@H](NC(=O)NN)CCNC(=O)[C@H]([C@@H](C)O)NC(=O)[C@H](CCNC(=O)OC(C)(C)C)NC(=O)[C@H](CCNC(=O)OC(C)(C)C)NC1=O]

Acetyl chloride (108.9 mg, 1.39 mmol) was added drop-wise in MeOH/water (30 m/1 ml). The solution was added to the step a product (1.16 g, 0.925 mmol). The reaction atmosphere was flushed with nitrogen, then Pd/C (dry basis, 270 mg) was added and the resulting mixture was stirred under hydrogen for 2 hours. Pd/C was filtered, and the filtrate was concentrated by rotary evaporation and purified by RPLC (150 g, 30 to 65% acetonitrile and water). Yield 1.02 g, 98.4%. Ions found by LCMS: [(M−2Boc+2H)/2]⁺=460.8, [(M−2Boc−tBu+2H)/2]⁺=432.6, [(M−3Boc+2H)/2]⁺=410.8.

### Step c

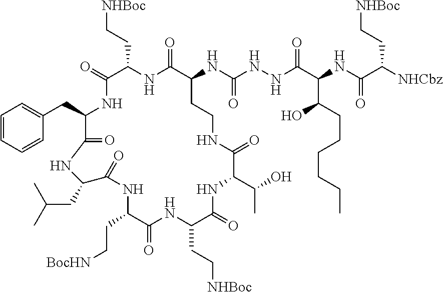 [SMILES: CCCCCC[C@@H](O)[C@H](NC(=O)[C@H](CCNC(=O)OC(C)(C)C)NC(=O)OCc1ccccc1)C(=O)NNC(=O)N[C@H]1CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](Cc2ccccc2)NC(=O)[C@H](CCNC(=O)OC(C)(C)C)NC1=O]

To a solution of a mixture of extended threonine-Dab-Z (366.5 mg, 0.7 mmol, analogous dipeptide described in Example 194, step c) and HATU (266.1 mg, 0.7 mmol) in DMF (1 ml) was added DIPEA (150 mg, 1.15 mmol). After stirred for 10 minutes, the reaction mixture was added with the step b product (560.2 mg, 0.5 mmol) and continued stirring at room temperature for 30 minutes. It was then purified by RPLC (100 g, 50 to 90% MeOH and water). Yield 559.6 mg, 73.4%. Ion found by LCMS: [(M−2Boc+2H)/2]⁺=713.5.

### Step d

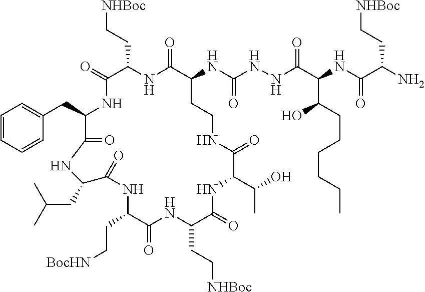 [SMILES: CCCCCC[C@@H](O)[C@H](NC(=O)[C@@H](N)CCNC(=O)OC(C)(C)C)C(=O)NNC(=O)N[C@H]1CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](Cc2ccccc2)NC(=O)[C@H](CCNC(=O)OC(C)(C)C)NC1=O]

A 50 ml reaction flask was filled with nitrogen and charged with the step c product (250 mg, 0.154 mmol) and MeOH (20 ml). Pd/C (dry basis, 100 mg) was then added, and the resulting mixture was stirred under hydrogen for 4 hours. Pd/C was filtered, and the filtrate was concentrated by rotary evaporation and purified by RPLC (100 g, 40 to 85% MeOH and water). Yield 125.2 mg, 54.5%. Ion found by LCMS: [(M−Boc+2H)/2]⁺=696.3, [(M−3Boc+2H)/2]⁺=596.3.

### Step e

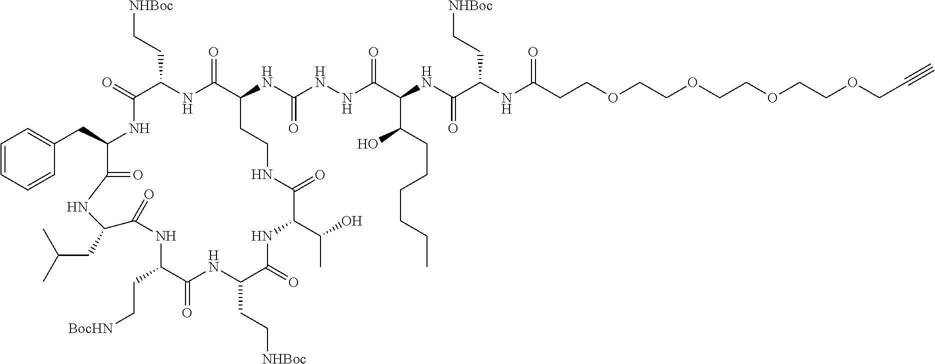 [SMILES: C#CCOCCOCCOCCOCCC(=O)N[C@@H](CCNC(=O)OC(C)(C)C)C(=O)N[C@H](C(=O)NNC(=O)N[C@H]1CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](Cc2ccccc2)NC(=O)[C@H](CCNC(=O)OC(C)(C)C)NC1=O)[C@H](O)CCCCCC]

To a solution of propargyl-PEG4-acid (31.2 mg, 0.12 mmol) in anhydrous DMF (0.5 ml) was added HATU (0.12 mmol) and DIPEA (52 mg, 0.4 mmol). After the resulting mixture was stirred for 10 minutes, it was added with the step-d product (125.2 mg, 0.0839 mmol). The reaction mixture was stirred for 1 hour and directly purified through RPLC (50 g, 40 to 95% MeOH and water). Yield 122 mg, 83.9%. Ion found by LCMS: [(M−2Boc+2H)/2]⁺=767.6.

### Step f

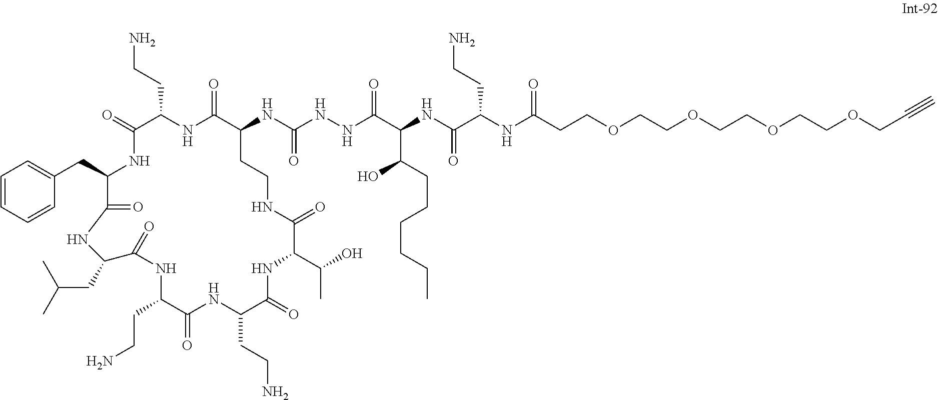 [SMILES: C#CCOCCOCCOCCOCCC(=O)N[C@@H](CCN)C(=O)N[C@H](C(=O)NNC(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCCCCC]

The step e product (122 mg, 0.0704 mmol) was dissolved in TFA (0.5 ml). The solution was stirred at room temperature for 20 minutes, concentrated, and directly purified by RPLC (50 g, 5 to 35% acetonitrile and water, using 0.1% TFA as modifier). Ions found by LCMS: [(M+2H)/2]⁺=667.5, [(M+3H)/3]⁺=445.4, [(M+4H)/4]⁺=334.2.

### Example 193—Synthesis of Int-93

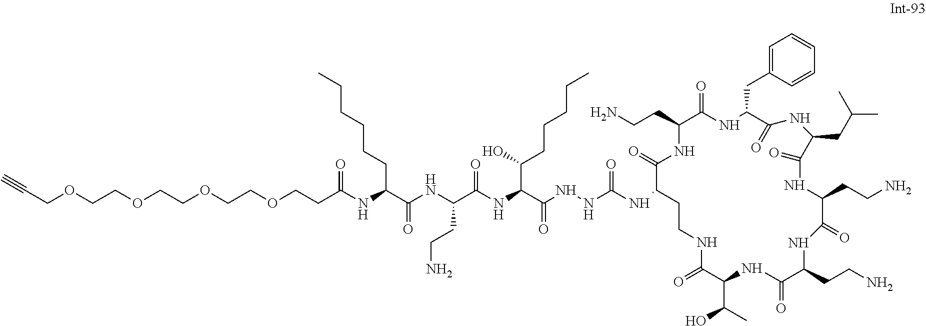 [SMILES: C#CCOCCOCCOCCOCCC(=O)N[C@@H](CCCCCC)C(=O)N[C@@H](CCN)C(=O)N[C@H](C(=O)NNC(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCCCC]

The title compound was prepared analogously to Example 194, where the (S)-methyl 2-aminohexanoate was substituted with (S)-methyl 2-aminooctanoate in step a. Ion(s) found by LCMS: [(M+2H)/2]⁺=752.4, [(M+3H)/3]⁺=501.9, [(M+4H)/4]⁺=376.7.

### Example 194—Synthesis of Int-94

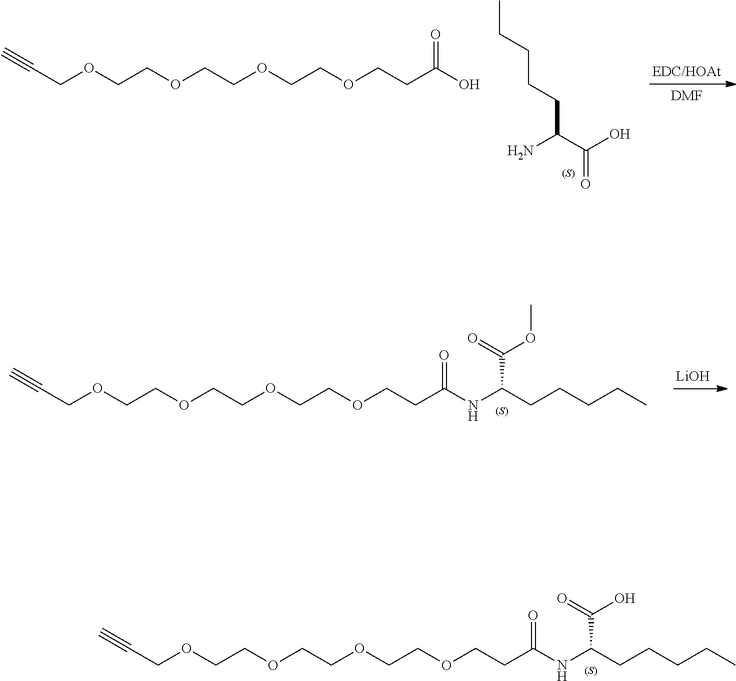

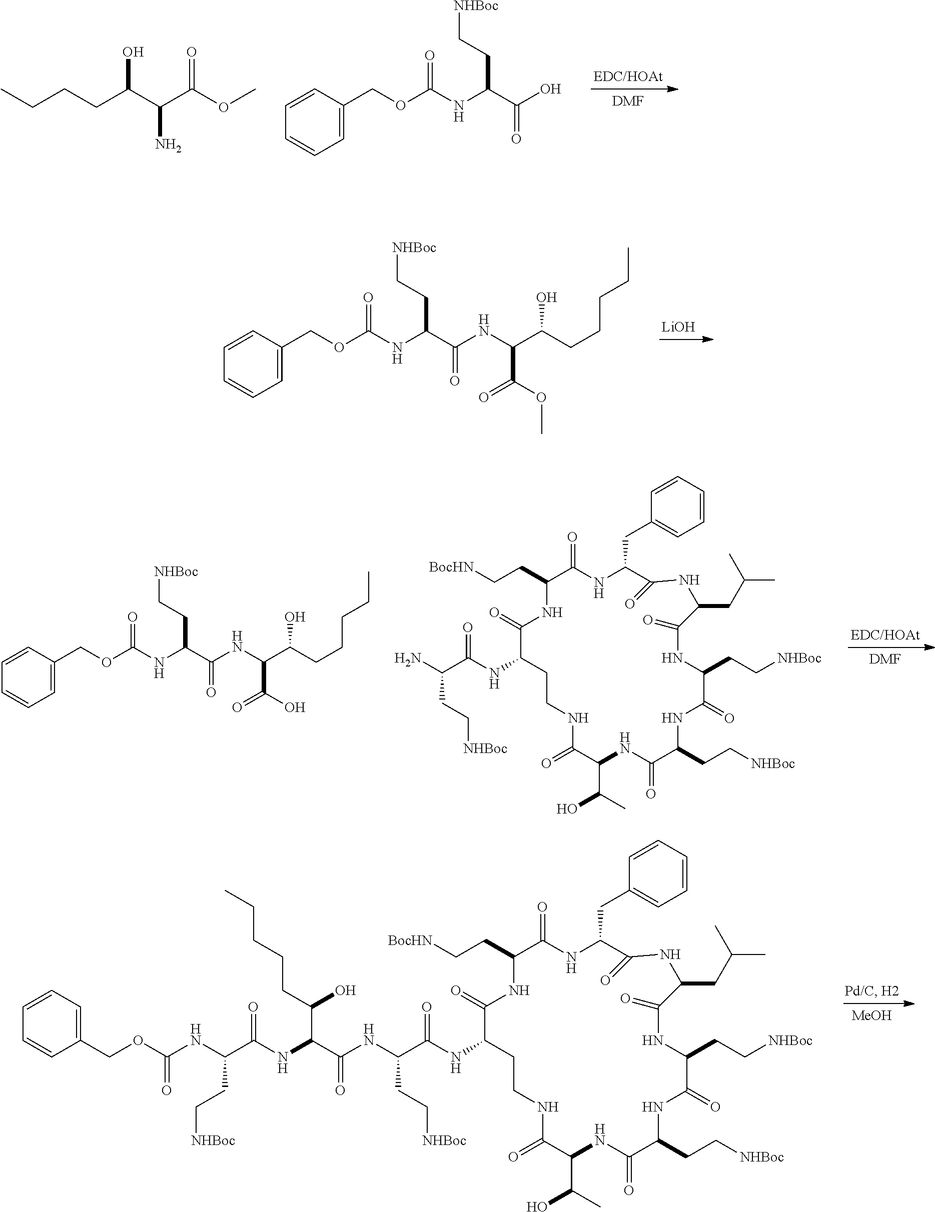

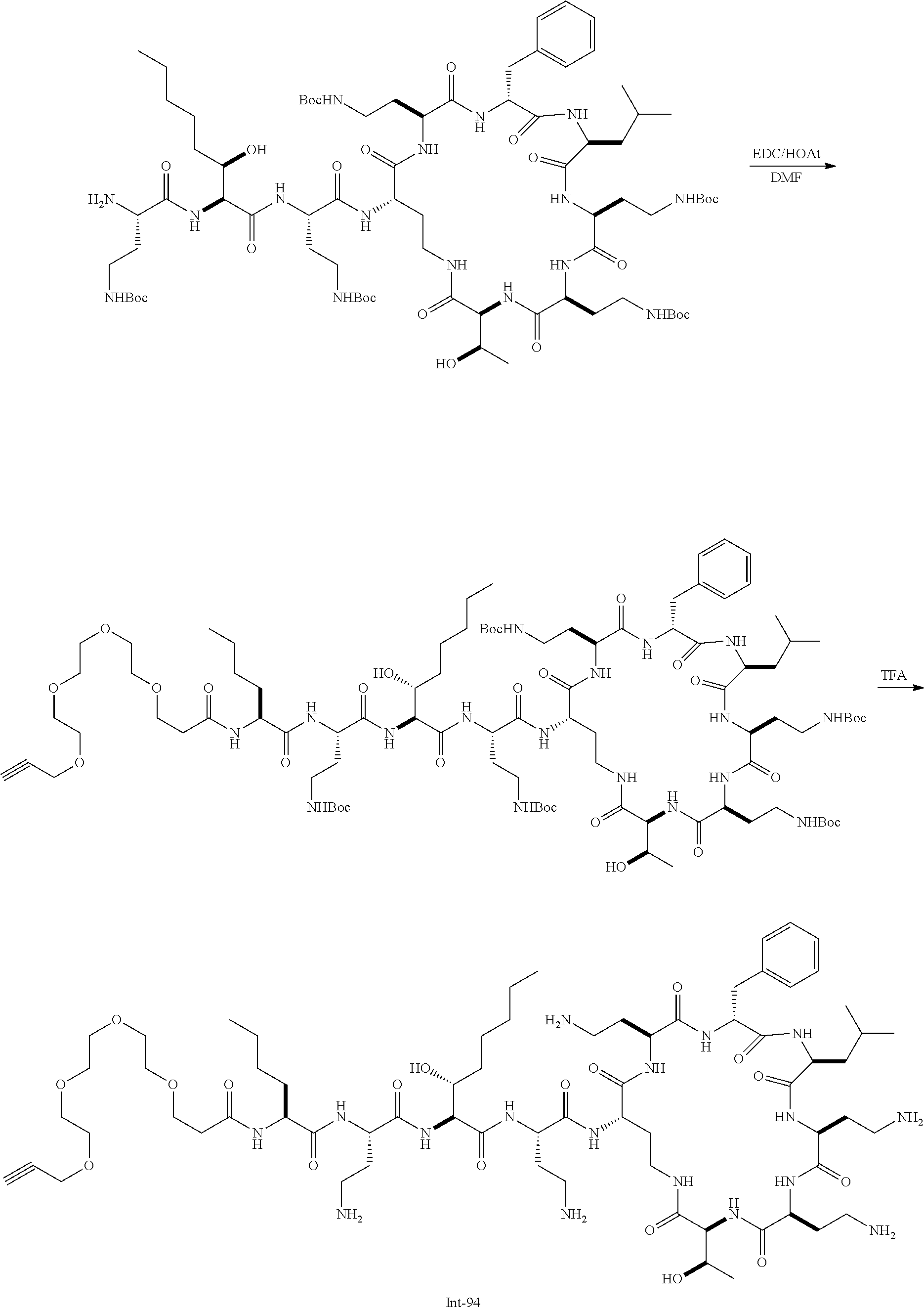

### Step a

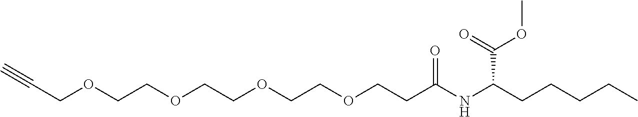 [SMILES: C#CCOCCOCCOCCOCCC(=O)N[C@@H](CCCCC)C(=O)OC]

To a solution of propargyl-PEG-4-acid (0.52 g 2 mmol), and (S)-methyl 2-aminoheptanoate (0.35 g, 2 mmol) in DMF (20 ml) was added EDC (0.60 g, 3 mmol), HOBT (0.45 g, 3 mmol), and DIEA (0.56 ml, 4 mmol) at room temperature. The solution was stirred overnight. The resulted solution was concentrated and purified by reverse phase liquid chromatography (RPLC) using an Isco CombiFlash liquid chromatograph eluted with 5% to 100% acetonitrile and water, using 0.1% TFA as the modifier. Ion(s) found by LCMS: (M+H)⁺=388.2.

### Step b

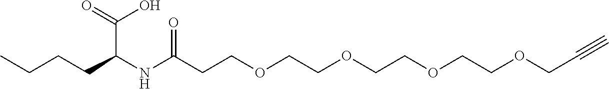 [SMILES: C#CCOCCOCCOCCOCCC(=O)N[C@@H](CCCC)C(=O)O]

Lithium hydroxide (60 mg, 2.5 mmol) in 2 ml water was added into the solution of the amino acid ester from step a (0.38 g, 1 mmol) in 2 ml MeOH and 2 ml THF. Progress of the reaction was monitored by LCMS. After completion of the reaction by LCMS, the crude solution was neutralized with Amberlite® IRN-77 ion exchange resin. The solution was filtered and the filtrate was concentrated and used for next step without purification. Ion(s) found by LCMS: (M+H)⁺=374.2.

### Step c

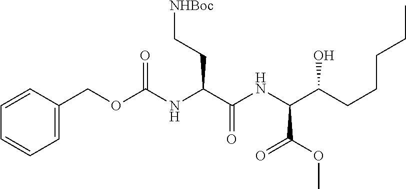 [SMILES: CCCCC[C@@H](O)[C@H](NC(=O)[C@H](CCNC(=O)OC(C)(C)C)NC(=O)OCc1ccccc1)C(=O)OC]

To a solution of (2S,3R)-methyl 2-amino-3-hydroxyheptanoate (0.9 g, 5 mmol, see Example 177), and (2S)-2-{[(benzyloxy) carbonyl]-amino}-4-[(tert-butoxycarbonyl)amino]butanoic acid (1.84 gmg, 5 mmol) in DMF (30 ml) was added EDC (1.2 g, 6 mmol), HOBT (0.8 g, 6 mmol), and DIPEA (1.4 ml, 10 mmol) at room temperature. The solution was stirred overnight. The reaction was concentrated and purified by reverse phase liquid chromatography (RPLC) using an Isco CombiFlash liquid chromatograph eluted with 5% to 100% acetonitrile and water, using 0.1% TFA as the modifier. Ion(s) found by LCMS: (M+H)⁺=524.3.

### Step d

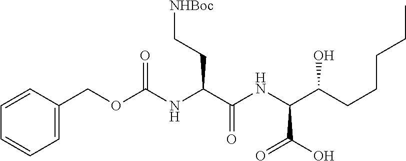 [SMILES: CCCCC[C@@H](O)[C@H](NC(=O)[C@H](CCNC(=O)OC(C)(C)C)NC(=O)OCc1ccccc1)C(=O)O]

Lithium hydroxide (60 mg, 2.5 mmol) in 2 ml water was added to the solution of the amino acid ester from step a (0.52 g, 1 mmol) in 2 ml MeOH and 2 ml THF. The reaction was monitored by LCMS. After the completion, the solution was neutralized with Amberlite® IRN-77, ion exchange resin. The mixture was filtered and the filtrate was concentrated and used in the next step without purification. Ion(s) found by LCMS: (M+H)⁺=510.3.

Steps e and f.

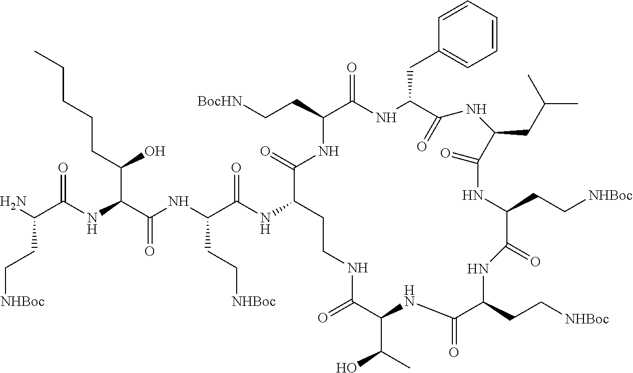 [SMILES: CCCCC[C@@H](O)[C@H](NC(=O)[C@@H](N)CCNC(=O)OC(C)(C)C)C(=O)N[C@@H](CCNC(=O)OC(C)(C)C)C(=O)N[C@H]1CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](Cc2ccccc2)NC(=O)[C@H](CCNC(=O)OC(C)(C)C)NC1=O]

To a solution of tetra-Boc Dab-polymyxin B cycloheptapeptide (1.06 g, 1 mmol) and (2S)-2-{[(benzyloxy) carbonyl)-amino}-4-[(tert-butoxycarbonyl)amino]butanoic acid (510 mg, 1 mmol) in DMF (20 ml) was added EDC (0.3 g, 1.5 mmol), HOBT (0.15 g, 1 mmol), DIEA (0.28 ml, 2 mmol) at room temperature. The solution was stirred overnight. The resulting solution was concentrated and re-dissolved into 2 ml methanol, then added drop-wise into 100 ml vigorously stirred water. This heterogeneous solution was stirred for 1 h, then filtered with Buchner funnel with filter paper. The collected crude product was dissolved in 20 ml methanol, then treated with 1 g of 5% Pd/C. The mixture was stirred at room temperature under a hydrogen atmosphere overnight. The palladium charcoal was removed by filtration. The filtrate was concentrated and purified by reverse phase liquid chromatography (RPLC) using an Isco CombiFlash liquid chromatograph eluted with 10% to 100% acetonitrile and water, using 0.1% TFA as the modifier. Yield of product as a TFA salt: 1.6 g, 85% yield. Ion(s) found by LCMS: [(M+2H)/2]⁺=810.5, [(M+3H)/3]⁺=540.7, [(M+4H)/4]⁺=405.7.

### Step g

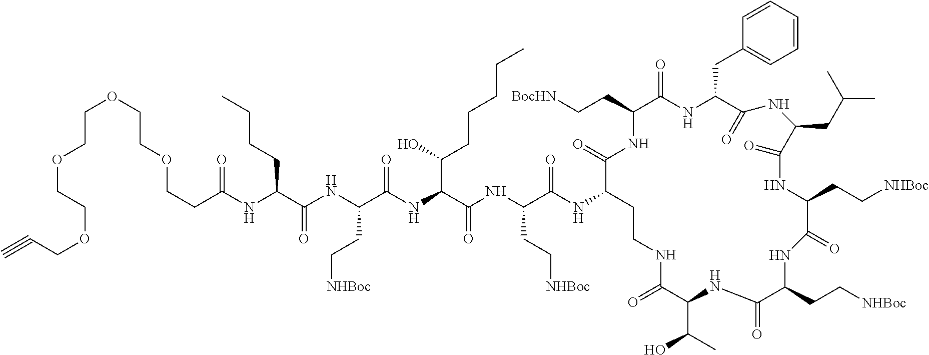 [SMILES: C#CCOCCOCCOCCOCCC(=O)N[C@@H](CCCC)C(=O)N[C@@H](CCNC(=O)OC(C)(C)C)C(=O)N[C@H](C(=O)N[C@@H](CCNC(=O)OC(C)(C)C)C(=O)N[C@H]1CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](Cc2ccccc2)NC(=O)[C@H](CCNC(=O)OC(C)(C)C)NC1=O)[C@H](O)CCCCC]

(S)-2-butyl-4-oxo-7,10,13,16-tetraoxa-3-azanonadec-18-yn-1-oic acid (400 mg, 1.1 mmol, from step b), and product from the previous step (1.6 g, 1 mmol) in DMF (10 ml) were added EDC (300 mg, 1.5 mmol), HOBT (150 mg, 1 mmol), and DIEA (0.28 ml, 2.0 mmol) at room temperature. The solution was stirred overnight. The resulting solution was concentrated and re-dissolved into 2 ml methanol. This solution was added dropwise to 50 ml vigorously stirred water, resulting in a heterogeneous solution that was stirred for 1 hour, then filtered through a Buchner funnel and filter paper. The crude product was dried and used in next step without further purification.

### Step h

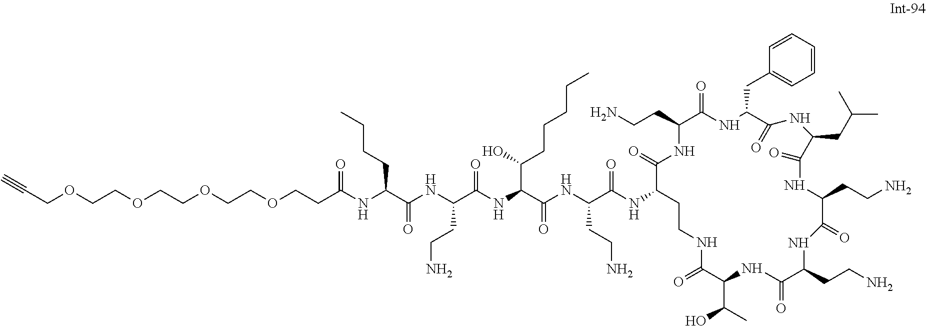 [SMILES: C#CCOCCOCCOCCOCCC(=O)N[C@@H](CCCC)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCCCC]

Product from step g was dissolved in 2 ml DCM and 2 ml TFA at room temperature. The solution was stirred 10 min, then concentrated and purified by reverse phase liquid chromatography (RPLC) using an Isco CombiFlash liquid chromatograph eluted with 0% to 100% acetonitrile and water, using TFA as the modifier. Yield of 1.20 g as a TFA salt, 61% yield. Ion(s) found by LCMS: [(M+2H)/2]⁺=737.9, [(M+3H)/3]⁺=492.3, [(M+4H)/4]⁺=369.5.

### Example 195—Synthesis of Int-95

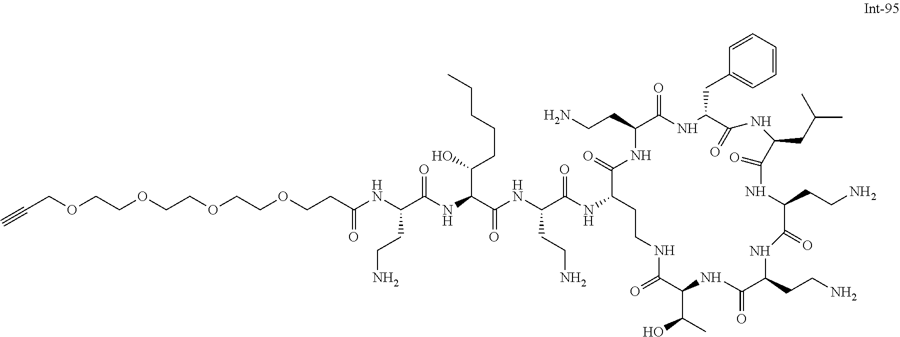 [SMILES: C#CCOCCOCCOCCOCCC(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCCCC]

The title compound was prepared analogously to Example 194, where the acid intermediate (used in step g) was substituted with propargyl-PEG4-acid. Ion(s) found by LCMS: [(M+2H)/2]⁺=681.4, [(M+3H)/3]⁺=454.6, [(M+4H)/4]⁺=341.2.

### Example 196—Synthesis of Int-96

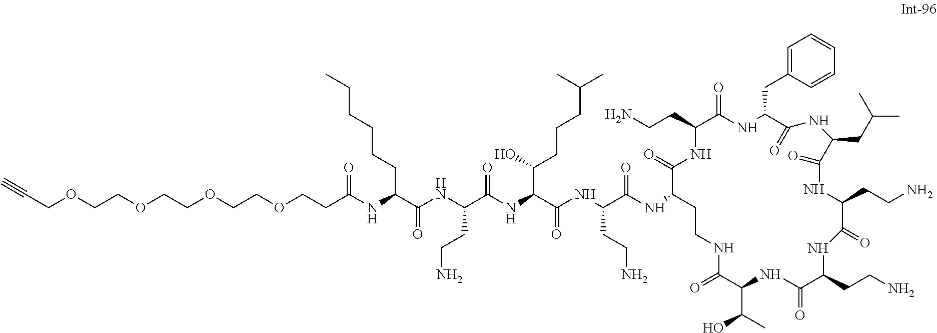 [SMILES: C#CCOCCOCCOCCOCCC(=O)N[C@@H](CCCCCC)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCCC(C)C]

The title compound was prepared analogously to Example 193, where the C4-extended threonine was substituted with extended threonine derived from 5-methylhexanal, in the step g. Synthesis of extended threonine derivatives is described in Example 177. Ion(s) found by LCMS: [(M+2H)/2]⁺=758.4, [(M+3H)/3]⁺=506.6, [(M+4H)/4]⁺=380.2.

### Example 197—Synthesis of Int-97

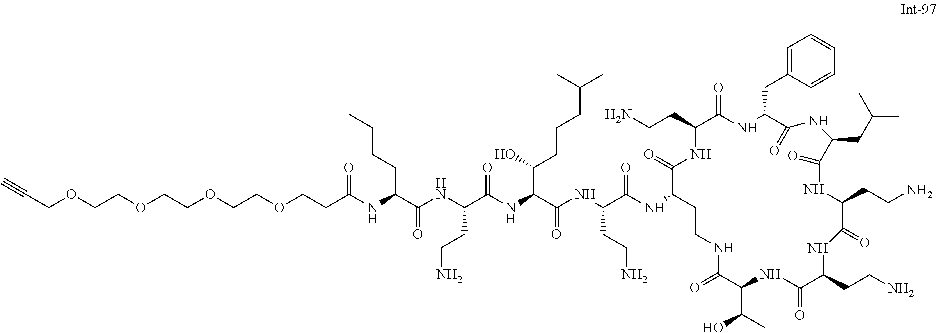 [SMILES: C#CCOCCOCCOCCOCCC(=O)N[C@@H](CCCC)C(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCCC(C)C]

The title compound was prepared analogously to Example 194, where the C4-extended threonine was replace with extended threonine derived from 5-methyl-hexanal in the step g. Synthesis of extended threonine derivatives is described in Example 177. Ion(s) found by LCMS: [(M+2H)/2]⁺=745.4, [(M+3H)/3]⁺=497.2, [(M+4H)/4]⁺=373.2.

### Example 198—Synthesis of Int-98

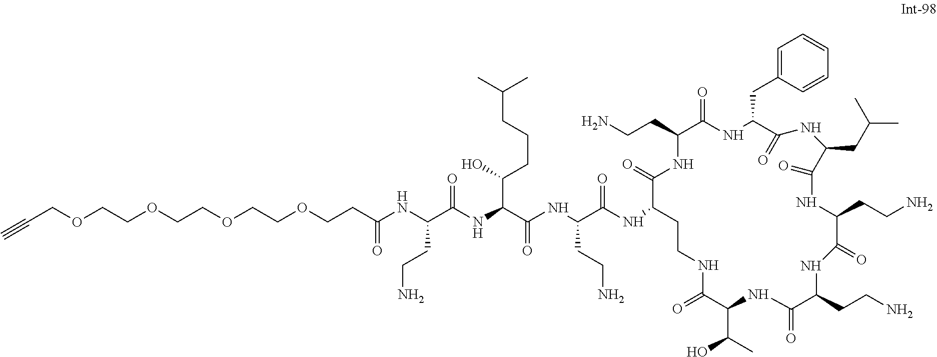 [SMILES: C#CCOCCOCCOCCOCCC(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCCC(C)C]

The title compound was prepared analogously to Example 195, where the C4-extended threonine was replaced with the extended threonine derived from 5-methyl-hexanal in the step g. Synthesis of extended threonine derivatives is described in Example 177. Ion(s) found by LCMS: [(M+2H)/2]⁺=688.4, [(M+3H)/3]⁺=459.3, [(M+4H)/4]⁺=344.7.

### Example 199—Synthesis of Int-99

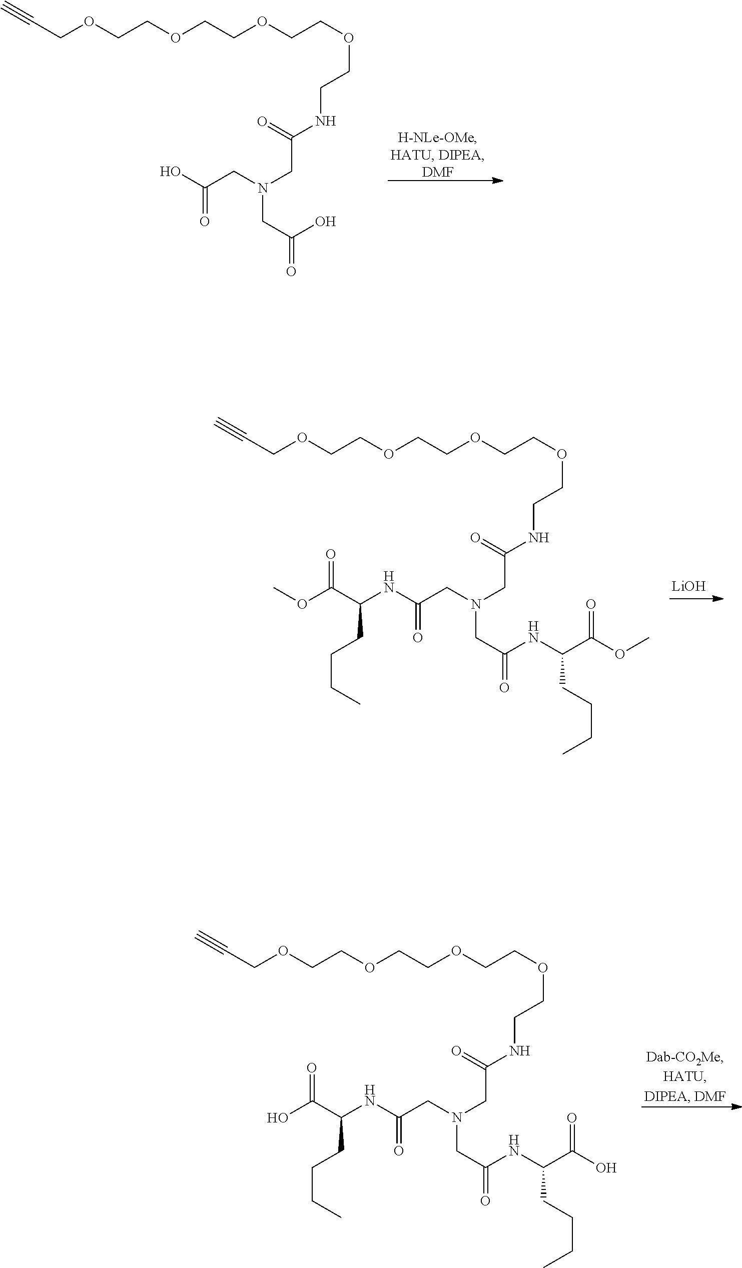

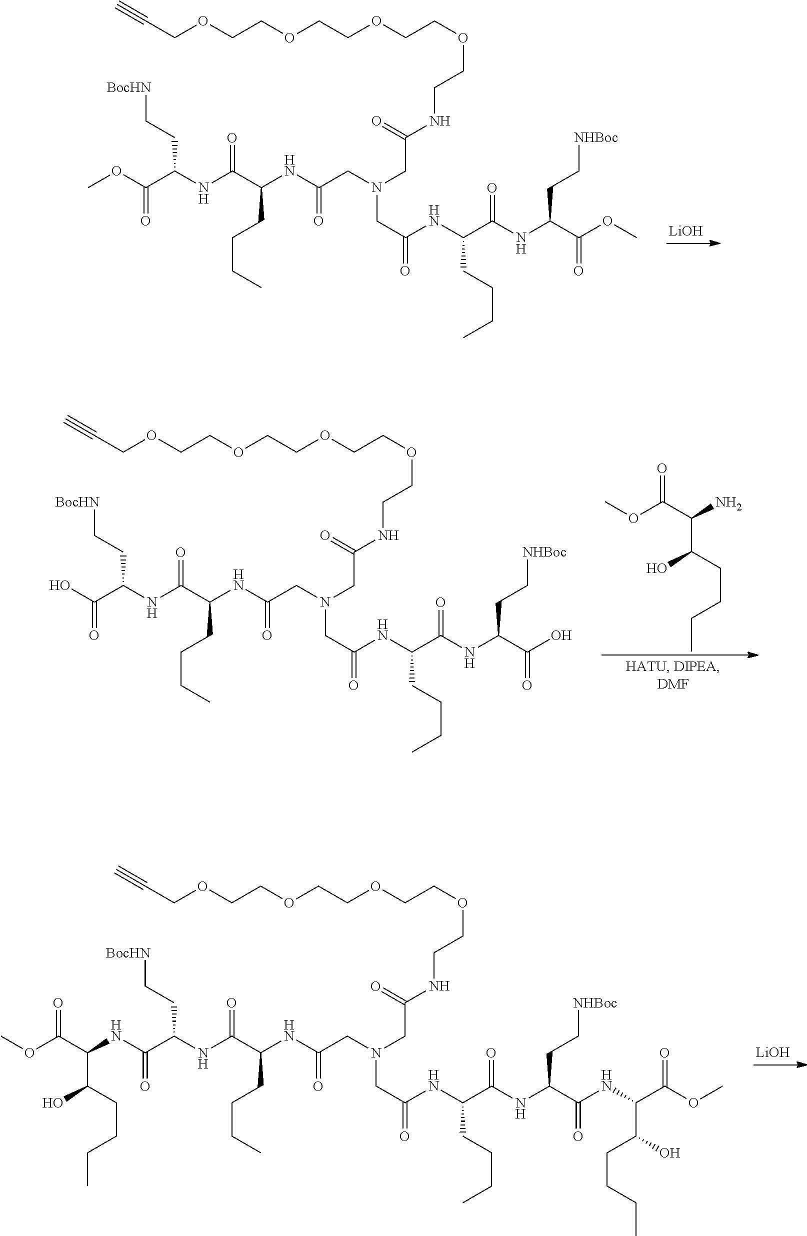

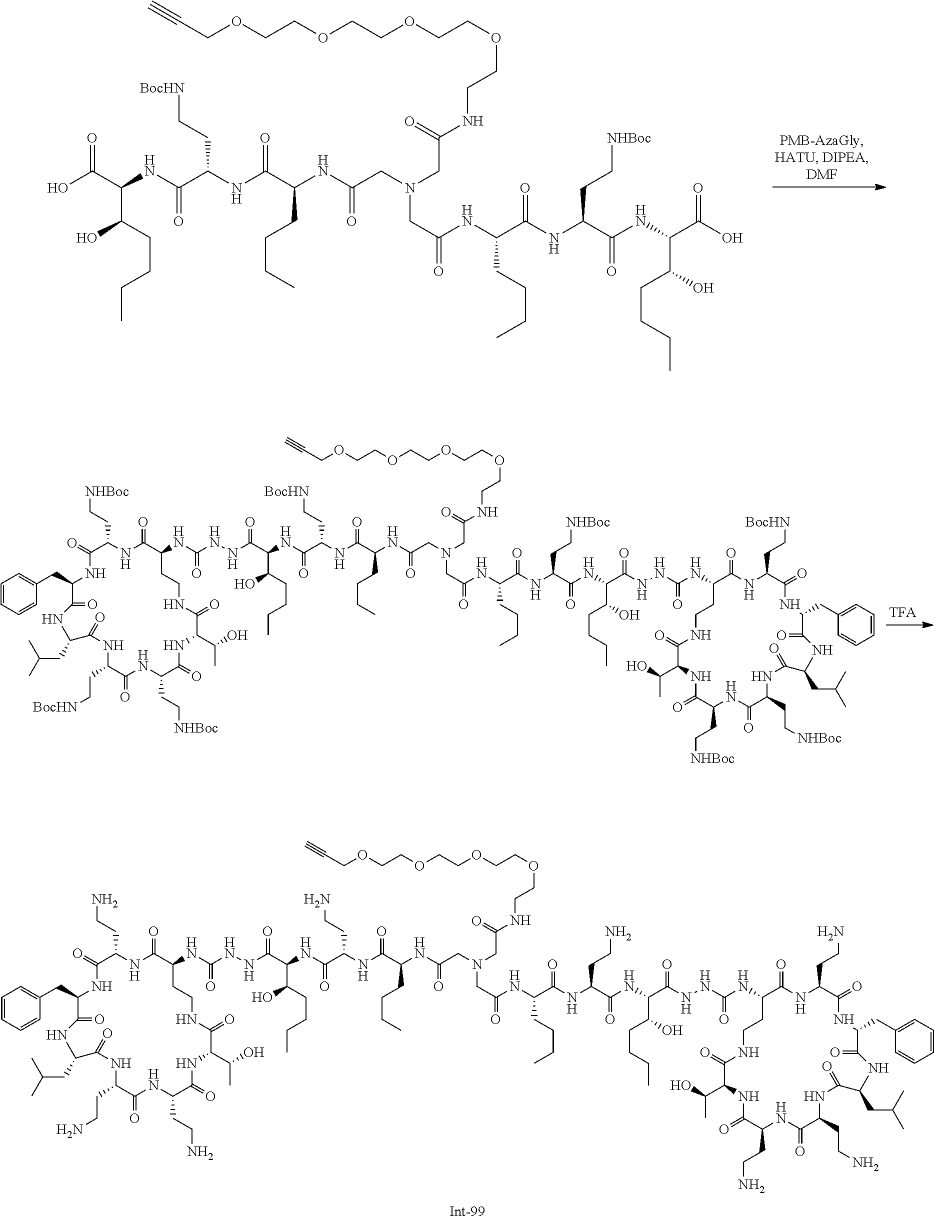

### Step a

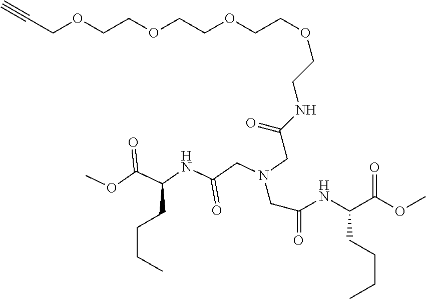 [SMILES: C#CCOCCOCCOCCOCCNC(=O)CN(CC(=O)N[C@@H](CCCC)C(=O)OC)CC(=O)N[C@@H](CCCC)C(=O)OC]

To a solution of the dicarboxlic acid (317 mg, 0.784 mmol) in anhydrous DMF (2 ml) was added H-NLe-OMe-HCl (345.2 mg, 1.9 mmol) and DIPEA (598 mg, 4.6 mmol). HATU (722.4 mg, 1.9 mmol) was then added in portions over 5 minutes. The reaction was stirred for 30 minutes and directly purified by RPLC (100 g column, 5 to 70% acetonitrile and water). Yield 238.4 mg, 46.2%. Ion found by LCMS: [M+H]⁺=659.4.

### Step b

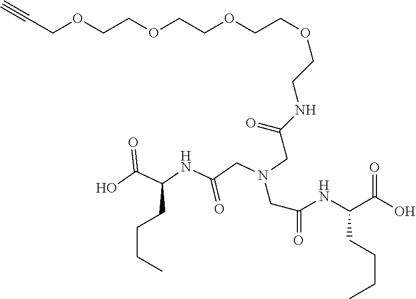 [SMILES: C#CCOCCOCCOCCOCCNC(=O)CN(CC(=O)N[C@@H](CCCC)C(=O)O)CC(=O)N[C@@H](CCCC)C(=O)O]

A solution of the step-a product (238.4 mg, 0.362 mmol) in MeOH (2 ml) was cooled in an ice-water bath and treated with portions a solution of LiOH (60.7 mg, 1.5 mmol) in water (2 ml) over 1 hour. The reaction was stirred for 3 more hours, then the pH was adjusted to neutral or slightly acidic by ion exchange Dowex 50W×8 hydrogen form. The resin was filtered, and the filtrate was concentrated to dryness. Yield 222.2 mg, 97.2%. Ion found by LCMS: [M+H]⁺=631.4.

### Step c

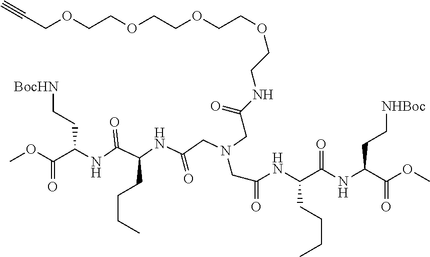 [SMILES: C#CCOCCOCCOCCOCCNC(=O)CN(CC(=O)N[C@@H](CCCC)C(=O)N[C@@H](CCNC(=O)OC(C)(C)C)C(=O)OC)CC(=O)N[C@@H](CCCC)C(=O)N[C@@H](CCNC(=O)OC(C)(C)C)C(=O)OC]

To a solution of the step b product (222.2 mg, 0.352 mmol) in anhydrous DMF (2 ml) was added (S)-methyl 2-amino-4-((tert-butoxycarbonyl)amino)butanoate HCl (242 mg, 0.9 mmol). After stirring to dissolve the amino acid, HATU (136.9 mg, 0.36 mmol) was added in portions over 5 minutes, followed by DIPEA (260 mg, 2 mmol). The reaction was stirred for 30 minutes and directly purified by RPLC (100 g column, 10 to 70% acetonitrile and water). Yield 159.9 mg, 42.9%. Ion found by LCMS: [(M−2Boc+2H)/2]⁺=430.1.

### Step d

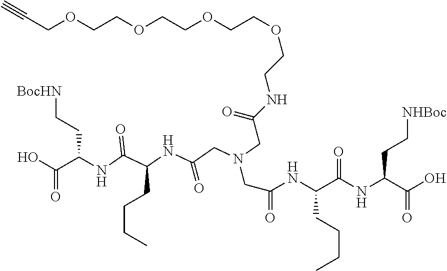 [SMILES: C#CCOCCOCCOCCOCCNC(=O)CN(CC(=O)N[C@@H](CCCC)C(=O)N[C@@H](CCNC(=O)OC(C)(C)C)C(=O)O)CC(=O)N[C@@H](CCCC)C(=O)N[C@@H](CCNC(=O)OC(C)(C)C)C(=O)O]

A solution of the step c product (159.9 mg, 0.151 mmol) in MeOH (1 ml) was cooled in an ice-water bath and treated in portions with a solution of LiOH (19 mg, 0.453 mmol) in water (1 ml) over 30 minutes. After the reaction was stirred for 3 more hours, its pH was adjusted to neutral by 4N HCl solution in dioxane and the solution was directly purified by RPLC (100 g column, 10 to 70% acetonitrile and water). Yield 122.6 mg, 78.7%. Ion found by LCMS: [(M−2Boc+2H)/2]⁺=416.4.

### Step e

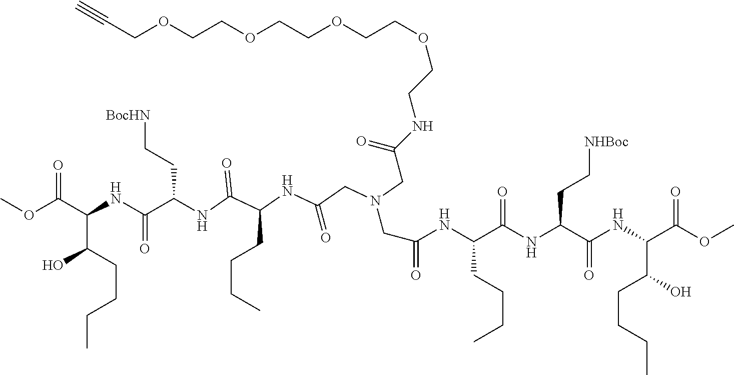

To a solution of the step d product (122.6 mg, 0.119 mmol) in anhydrous DMF (1 ml) was added C3-extended threonine (79 mg, 0.45 mmol, described in Example 177). After stirring to dissolve the amino acid, HATU (45.6 mg, 0.12 mmol) was added in portions over 5 minutes, followed by DIPEA (52 mg, 0.4 mmol). The reaction was stirred for 30 minutes and directly purified by RPLC (100 g column, 10 to 70% acetonitrile and water). Yield 122.6 mg, 76.6%. Ion found by LCMS: [(M−2Boc+2H)/2]⁺=573.4.

### Step f

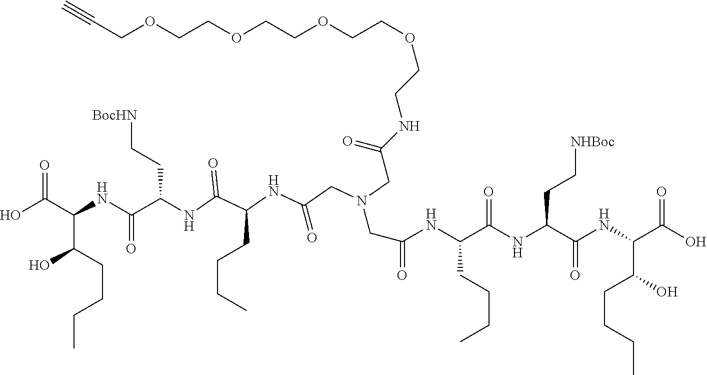 [SMILES: C#CCOCCOCCOCCOCCNC(=O)CN(CC(=O)N[C@@H](CCCC)C(=O)N[C@@H](CCNC(=O)OC(C)(C)C)C(=O)N[C@H](C(=O)O)[C@H](O)CCCC)CC(=O)N[C@@H](CCCC)C(=O)N[C@@H](CCNC(=O)OC(C)(C)C)C(=O)N[C@H](C(=O)O)[C@H](O)CCCC]

A solution of the step e product (122.6 mg, 0.091 mmol) in MeOH (1 ml) was cooled in an ice-water bath and treated with portions of a solution of LiOH (16.8 mg, 0.4 mmol) in water (1 ml) over 30 minutes. After the reaction was stirred for 3 hours, its pH was adjusted to neutral by 4N HCl solution in dioxane and the solution was directly purified by RPLC (100 g column, 10 to 70% acetonitrile and water). Yield 112.8 mg, 92%. Ion found by LCMS: [(M−2Boc+2H)/2]⁺=559.4.

### Step q

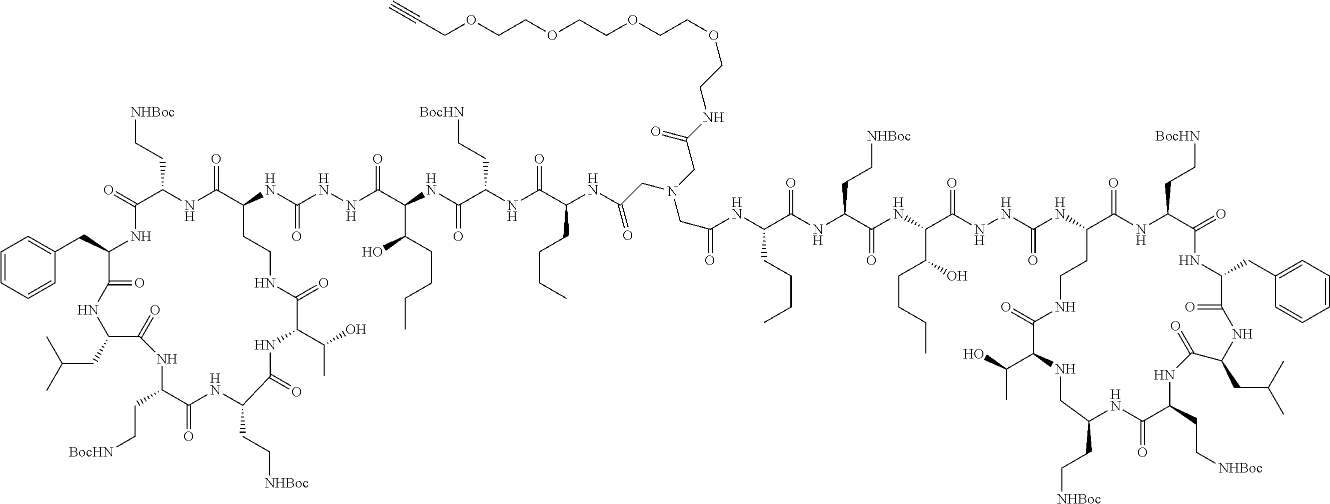

To a solution of a mixture of the step-f product (101.9 mg, 0.0773 mmol) and PMB-AzaGly-NH2 (190.6 mg, 0.17 mmol, see Example 192, step b) in anhydrous DMF (1.5 ml) was added HATU (60.2 mg, 0.159 mmol). After stirring to dissolve all the solids, the reaction mixture was treated with DIPEA (52 mg, 0.4 mmol) and stirred for 30 minutes. It was then purified by RPLC (100 g column, 60 to 95% MeOH and water, using 0.1% TFA as modifier). Yield 168.5 mg, 61.9%. Ions found by LCMS: [(M−2Boc+3H)/3]⁺=1108.3. [(M−3Boc+3H)/3]⁺=1074.6.

### Step h

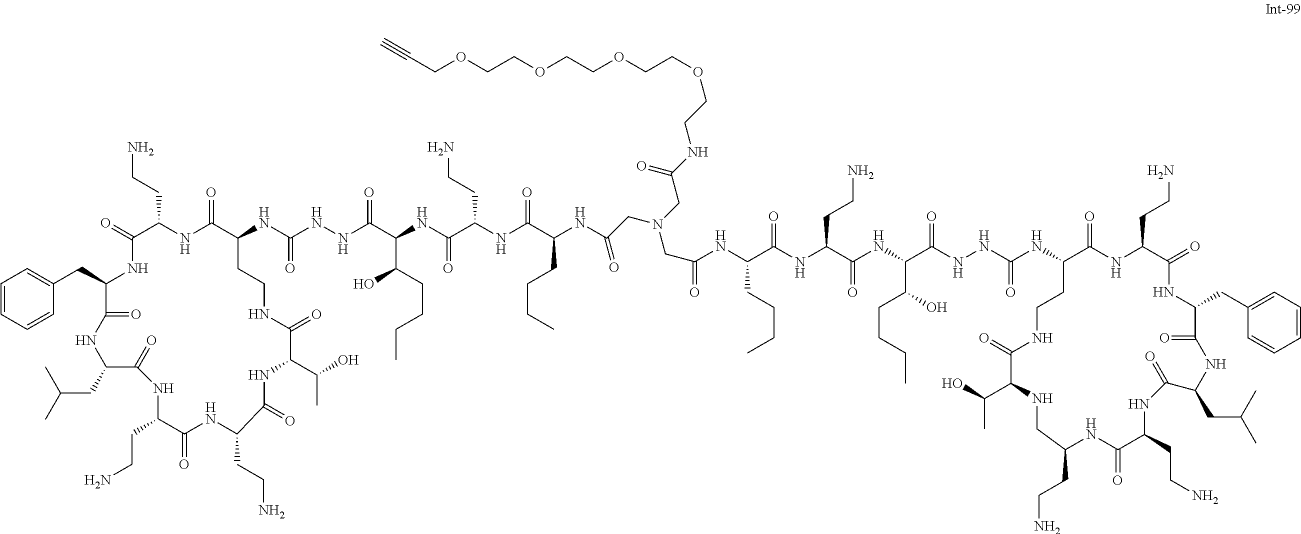 [SMILES: C#CCOCCOCCOCCOCCNC(=O)CN(CC(=O)N[C@@H](CCCC)C(=O)N[C@@H](CCN)C(=O)N[C@H](C(=O)NNC(=O)N[C@H]1CCNC(=O)[C@H]([C@@H](C)O)NC[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCCC)CC(=O)N[C@@H](CCCC)C(=O)N[C@@H](CCN)C(=O)N[C@H](C(=O)NNC(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCCC]

The step g product (168.5 mg, 0.0479 mmol) was dissolved in TFA (0.5 ml). The solution was stirred for 30 minutes, concentrated, and directly purified by HPLC (0 to 20% acetonitrile and water, using 0.1% TFA as modifier). Yield 75 mg, 43.1%. Ions found by LCMS: [(M+3H)/3]+=907.8, [(M+4H)/4]⁺=681.1, [(M+5H)/5]⁺=545.2, [(M+6H)/6]⁺=454.5, [(M+7H)/7]⁺=389.6.

### Example 200—Synthesis of Int-100

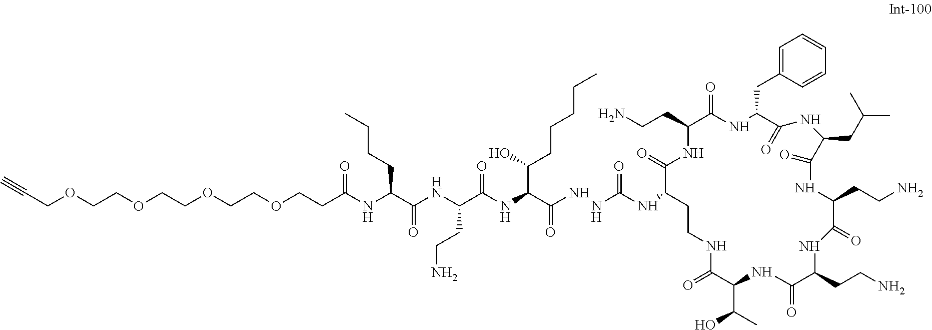 [SMILES: C#CCOCCOCCOCCOCCC(=O)N[C@@H](CCCC)C(=O)N[C@@H](CCN)C(=O)N[C@H](C(=O)NNC(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCCCC]

The title compound was prepared analogously to Example 178, where the L-threonine component is replaced with extended threonine derived from hexanal. Ion(s) found by LCMS: [(M+2H)/2]⁺=716.9, [(M+3H)/3]⁺=478.3, [(M+4H)/4]⁺=358.9.

### Example 201—Synthesis of Int-101

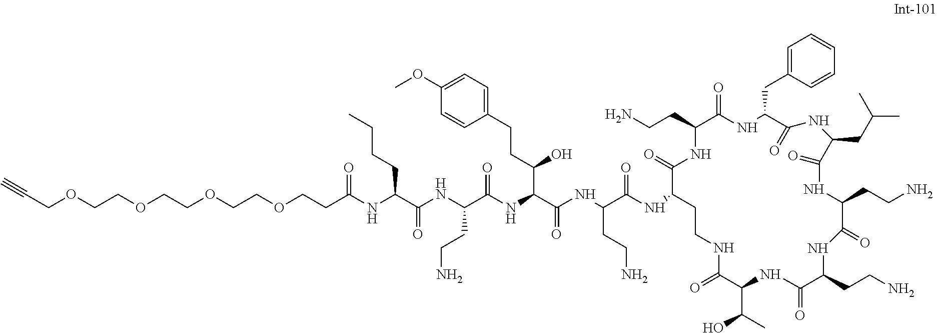 [SMILES: C#CCOCCOCCOCCOCCC(=O)N[C@@H](CCCC)C(=O)N[C@@H](CCN)C(=O)N[C@H](C(=O)NC(CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCc1ccc(OC)cc1]

The title compound was prepared analogously to Example 185, where the C5-extended threonine is replaced with extended threonine derived from 3-(4-methoxyl-phenyl)propionldehyde. Synthesis of extended threonine derivatives is described in Example 177. Ion(s) found by LCMS: [(M+2H)/2]⁺=770.4, [(M+3H)/3]⁺=513.9, [(M+4H)/4]⁺=385.7.

### Example 202—Synthesis of Int-102

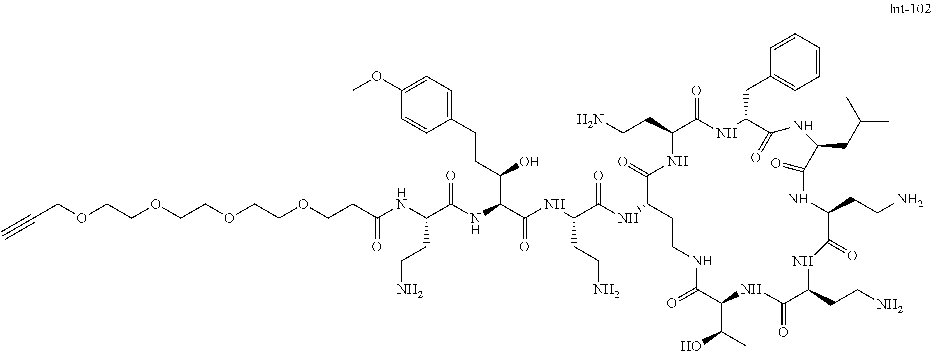 [SMILES: C#CCOCCOCCOCCOCCC(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCc1ccc(OC)cc1]

The title compound was prepared analogously to Example 201, where the acid intermediate was replaced with propargyl-PEG-4-acid in step g. Ion(s) found by LCMS: [(M+2H)/2]⁺=713.4, [(M+3H)/3]⁺=475.9, [(M+4H)/4]⁺=357.2.

### Example 202—Synthesis of Int-103

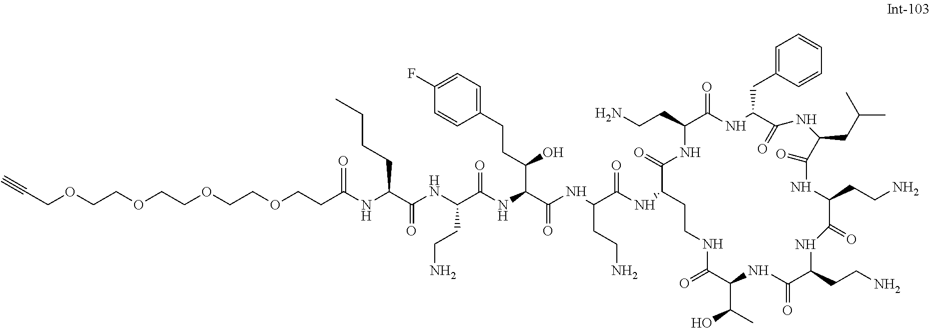 [SMILES: C#CCOCCOCCOCCOCCC(=O)N[C@@H](CCCC)C(=O)N[C@@H](CCN)C(=O)N[C@H](C(=O)NC(CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCc1ccc(F)cc1]

The title compound was prepared analogously to Example 185, where the C5 extended threonine was substituted with the extended threonine derived from 3-(4-fluoro-phenyl)propionldehyde. Synthesis of extended threonine derivatives is described in Example 177. Ion(s) found by LCMS: [(M+2H)/2]⁺=763.9, [(M+3H)/3]⁺=509.6, [(M+4H)/4]⁺=382.5.

### Example 204—Synthesis of Int-104

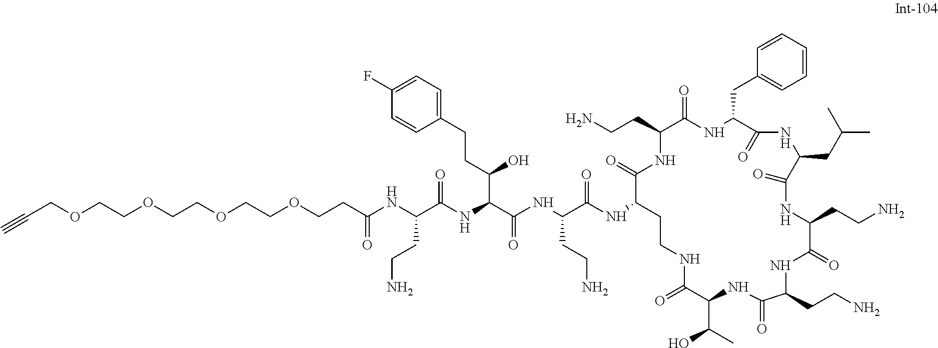 [SMILES: C#CCOCCOCCOCCOCCC(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCc1ccc(F)cc1]

The title compound was prepared analogously to Example 203, where the acid intermediate was substituted with propargyl-PEG-4-acid in the step g. Ion(s) found by LCMS: [(M+2H)/2]⁺=707.4, [(M+3H)/3]⁺=471.9, [(M+4H)/4]⁺=354.2

### Example 205—Synthesis of Int-105

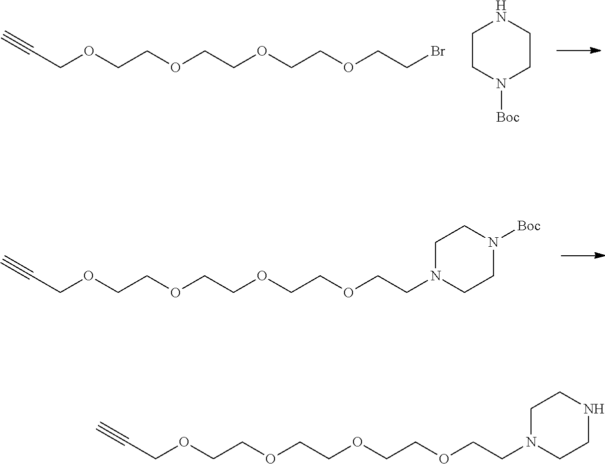

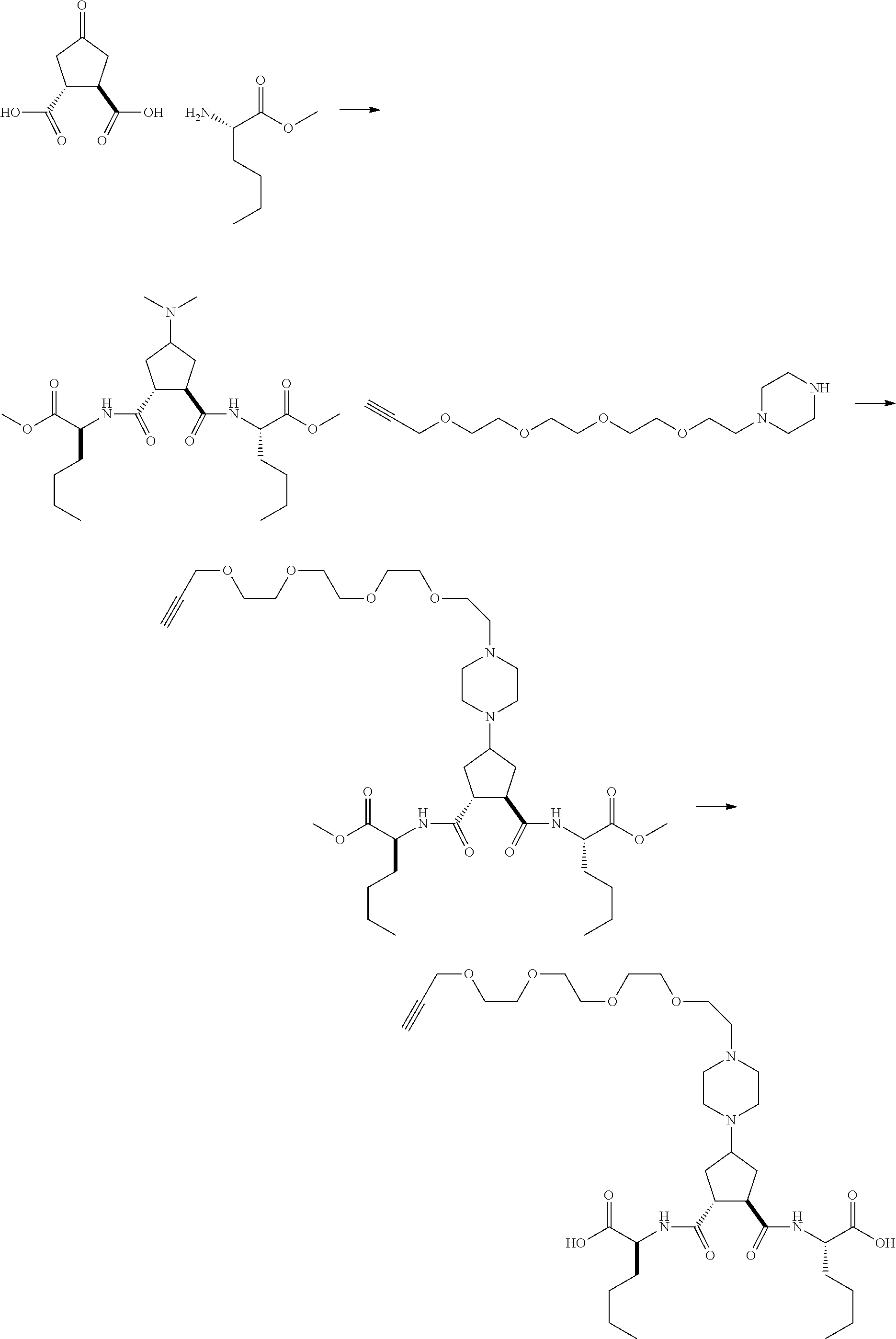

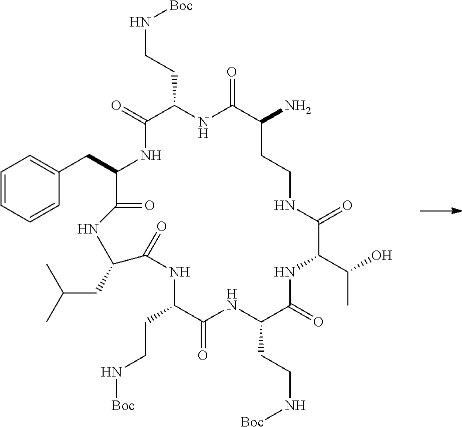 [SMILES: CC(C)C[C@@H]1NC(=O)[C@@H](Cc2ccccc2)NC(=O)[C@H](CCNC(=O)OC(C)(C)C)NC(=O)[C@@H](N)CCNC(=O)[C@H]([C@@H](C)O)NC(=O)[C@H](CCNC(=O)OC(C)(C)C)NC(=O)[C@H](CCNC(=O)OC(C)(C)C)NC1=O]

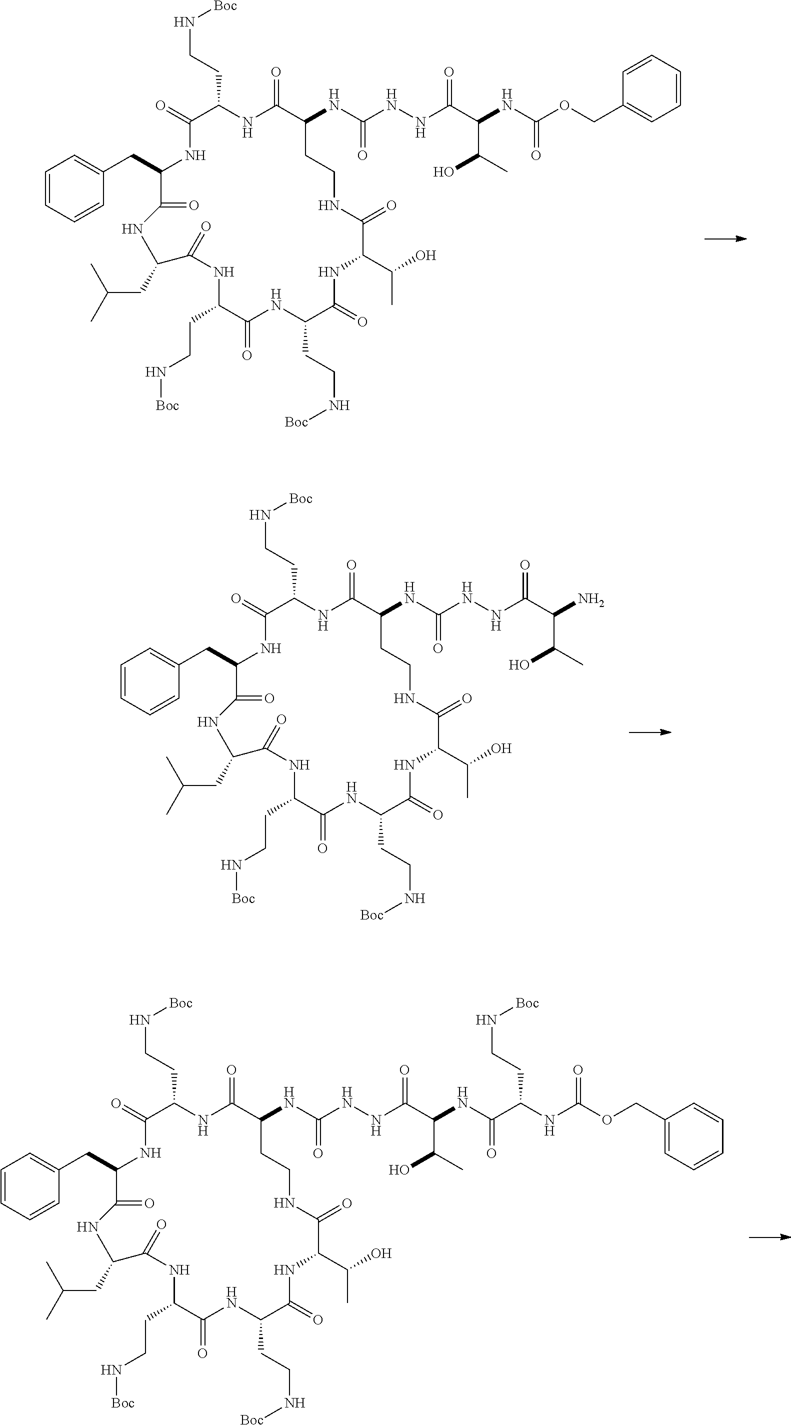

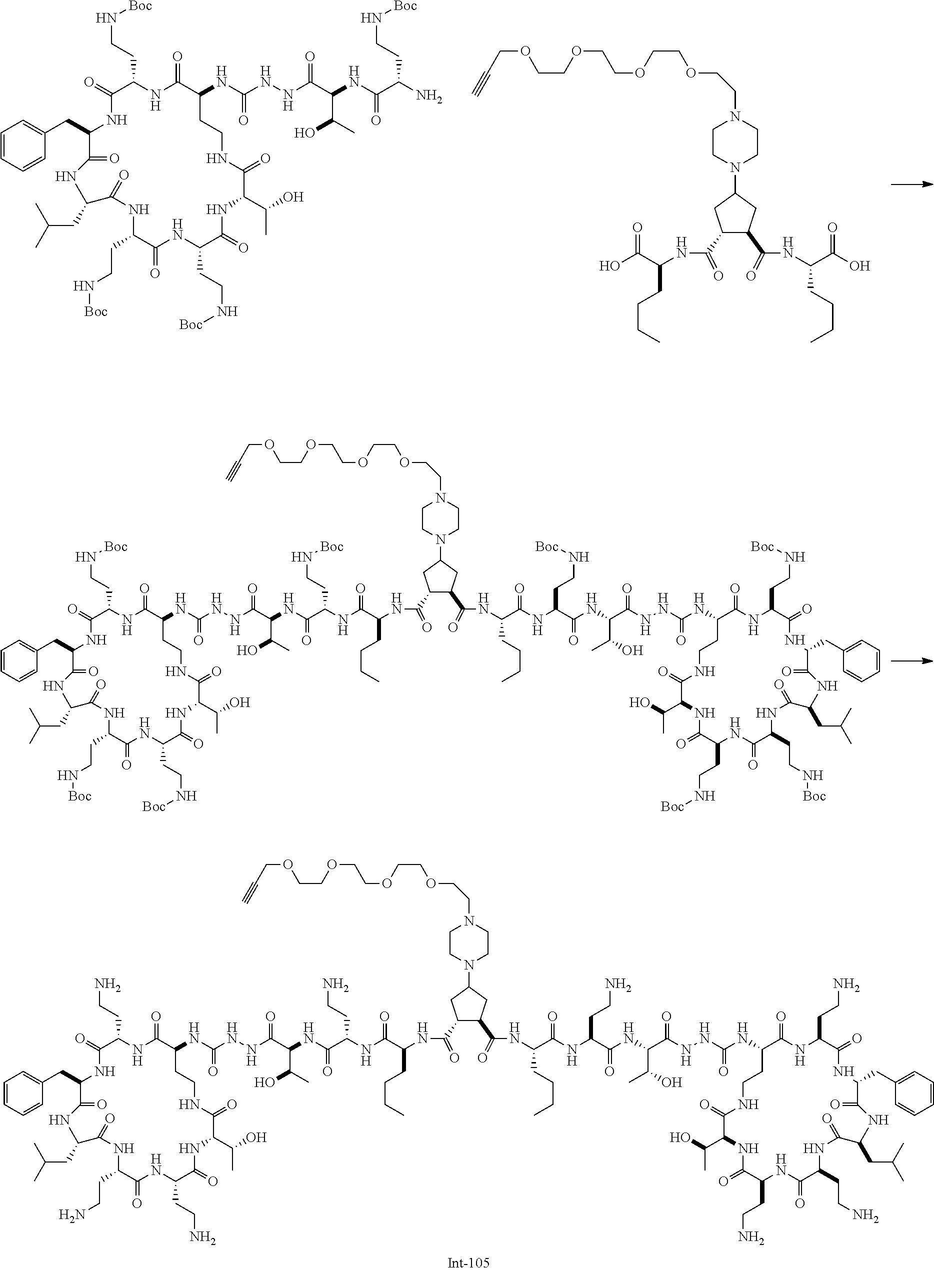

### Step a

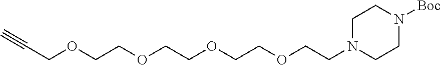 [SMILES: C#CCOCCOCCOCCOCCN1CCN(C(=O)OC(C)(C)C)CC1]

A solution of Boc-piperazine (0.316 g, 1.70 mmol), propargyl-PEG-4-bromide (0.500 g, 1.70 mmol), and potassium carbonate (0.352 g, 2.55 mmol) dissolved in acetonitrile (8 mL) was heated in a 65° C. oil bath for 15 h. The reaction was concentrated and purified by reversed phase liquid chromatography (RPLC) using an Isco CombiFlash liquid chromatograph eluted with 10% to 100% acetonitrile and water, using 0.1% TFA as the modifier. Yield of mono-TFA salt 0.837 g, 96% yield. Ion(s) found by LCMS: (M+H)⁺=401.4

### Step b

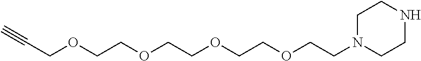 [SMILES: C#CCOCCOCCOCCOCCN1CCNCC1]

A solution of Boc-piperazine-PEG-4-alkyne (0.837 g, 1.63 mmol), and TFA (5 mL) was stirred for 5 minutes, concentrated under vacuum, and purified by reversed phase liquid chromatography (RPLC) using an Isco CombiFlash liquid chromatograph eluted with 10% to 100% acetonitrile and water, using 0.1% TFA as the modifier. Yield of bis-TFA salt 0.783 g, 91% yield. Ion(s) found by LCMS: (M+H)⁺=301.4

### Step c

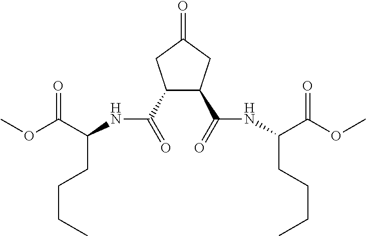

A solution of racemic trans-cyclopentanone-3, 4-dicarboxylic acid (0.500 g, 2.90 mmol), L-norleucine methylester HCl (1.16 g, 6.39 mmol), DIEA (3.03 mL, 17.4 mmol), and HATU (2.43 g, 6.39 mmol) in DMF (5 mL) was stirred at room temperature for 1 hr. The diastereomeric products were separated by reversed phase liquid chromatography (RPLC) using an Isco CombiFlash liquid chromatograph eluted with 10% to 100% acetonitrile and water, using 0.1% TFA as the modifier. The diastereomer with the shorter retention time was collected and taken on in the sequence. Yield of desired diastereomer 0.419 g, 34% yield. Ion(s) found by LCMS: did not ionize.

### Step d

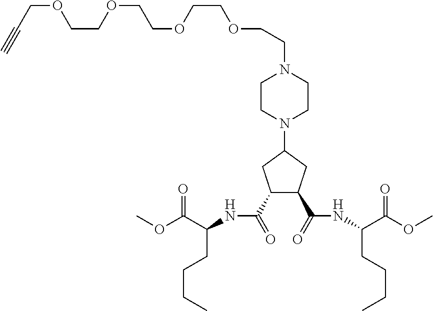 [SMILES: C#CCOCCOCCOCCOCCN1CCN(C2C[C@@H](C(=O)N[C@@H](CCCC)C(=O)OC)[C@H](C(=O)N[C@@H](CCCC)C(=O)OC)C2)CC1]

A solution of product from the previous step (0.280 g, 0.657 mmol), piperazine-PEG4-alkyne (0.416 g, 0.788 mmol), acetic acid (75 μL, 1.313 mmol), and dichloromethane (2 mL) was stirred for 30 min at room temperature, then treated with sodium triacetoxyborohydride (0.278 g, 1.31 mmol) in two equal portions 60 minutes apart. The reaction was stirred overnight, then purified by reversed phase liquid chromatography (RPLC) using an Isco CombiFlash liquid chromatograph eluted with 10% to 100% acetonitrile and water, using 0.1% TFA as the modifier. Yield of product was 0.275 g, 45% yield. Ion(s) found by LCMS: (M+H)⁺=711.4

### Step e

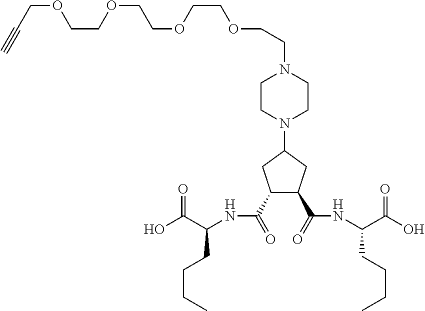 [SMILES: C#CCOCCOCCOCCOCCN1CCN(C2C[C@@H](C(=O)N[C@@H](CCCC)C(=O)O)[C@H](C(=O)N[C@@H](CCCC)C(=O)O)C2)CC1]

A solution of product from the previous step (0.275 g, 0.293 mmol) dissolved in THF (4 mL), was treated with a solution of lithium hydroxide (0.042 g, 1.76 mmol) dissolved in water (2 mL). LCMS after 30 min showed complete hydrolysis. The reaction was made slightly acidic with concentrated HCl, then purified by reversed phase liquid chromatography (RPLC) using an Isco CombiFlash liquid chromatograph eluted with 10% to 100% acetonitrile and water, using no modifier. Yield of product was 0.170 g, 85% yield. Ion(s) found by LCMS: (M+H)⁺=683.0

### Step f

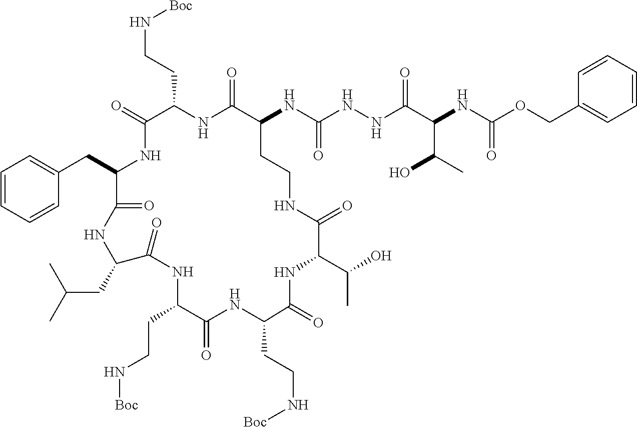

A mixture of Cbz-L-threonine-hydrazide (4 g, 8.23 mmol), CDI (1.33 g, 8.23 mmol), DMAP (0.067 g, 0.549 mmol, and N-Methyl morpholine (0.905 mL, 8.23 mmol), where suspended in DMF (20 mL) and heated in a 80 C oil bath until a homogeneous solution formed (˜15 min). To this solution was added polymyxin B heptapeptide (2.91 g, 2.74 mmol, described in Example 2). After stirring overnight the reaction was concentrated to an oil from which unreacted threonine hydrazide precipitates. The reaction was filtered, concentrated and purified by reversed phase liquid chromatography (RPLC) using an Isco CombiFlash liquid chromatograph eluted with 30% to 75% acetonitrile and water, using 0.1% TFA as the modifier. Yield of product was 3.2 g, 86% yield. Ion(s) found by LCMS: [M−1(Boc)/2]+1=561.4, [M−2(Boc)/2]+1=511.4, [M−3(Boc)/2]+1=461.4.

### Step g

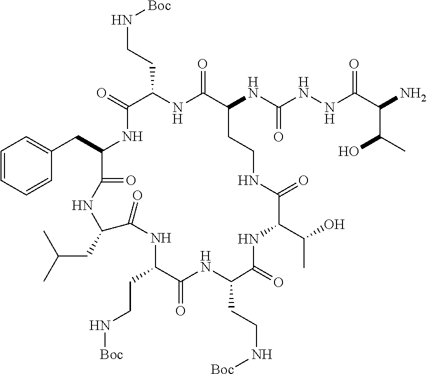

Methanol (30 mL) and acetyl chloride (0.173 mL, 2.44 mmol) where mixed to form an HCl/MeOH solution. This solution was added to a flask charged with 5% Pd/C (1 g) and starting material (3.01 g, 2.22 mmol). The reaction was vacuum flushed with hydrogen and stirred with hydrogen from a balloon until complete by LCMS (˜2 h), then filtered through celite, concentrated to a solid and used in the next step without additional purification. Yield 2.59 g, 96%. Ion(s) found by LCMS: [M−1(Boc)/2]+1=561.4, [M−2(Boc)/2]+1=511.4. [M−3(Boc)/2]+1=461.4.

### Step h

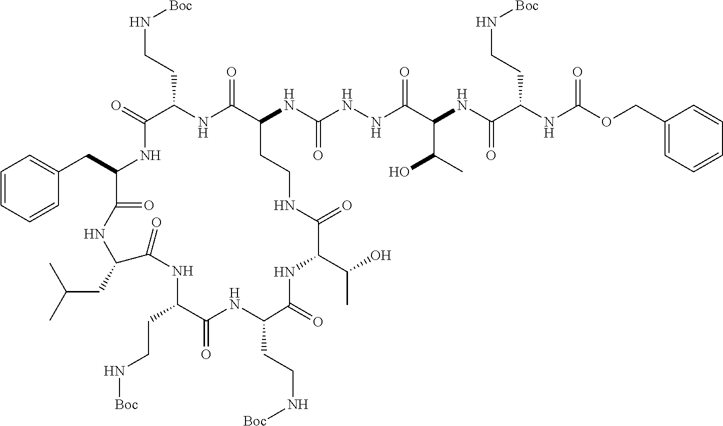

A solution of product from the previous step (3.5 g, 2.87 mmol) dissolved in DMF (15 mL) was charged with Z-L-Dab(Boc)-OH (1.11 g 3.15 mmol), DIEA (1.75 mL, 10.0 mmol) and HATU (1.20 g, 3.15 mmol) at room temperature while stirring. After 30 minutes, the reaction was concentrated and purified by reversed phase liquid chromatography (RPLC) using an Isco CombiFlash liquid chromatograph eluted with 40% to 100% acetonitrile and water, using 0.1% TFA as the modifier. Yield of product was 3.53 g, 79% yield. Ion(s) found by LCMS: [M−2(Boc)/2]+1=678.4

### Step i

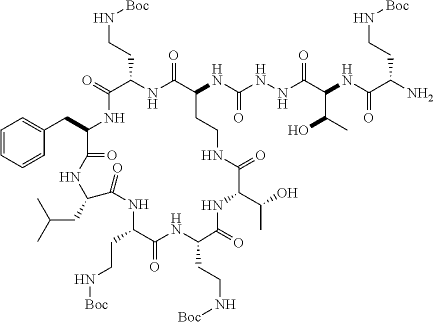

A solution of methanol (30 mL) and acetyl chloride (0.177 mL, 2.50 mmol) where mixed to form an HC/MeOH solution. This solution was added to a flask charged with 5% Pd/C (1.5 g) and starting material (3.53 g, 2.269 mmol). The reaction was vacuum flushed with hydrogen and stirred with hydrogen from a balloon until complete by LCMS (˜2 h), then filtered through celite, concentrated to a solid and used in the next step without additional purification. Yield 2.91 g, 90%. Ion(s) found by LCMS: [M−1(Boc)/2]+1=661.7, [M−2(Boc)/2]+1=611.2, [M−3(Boc)/2]+1=561.6.

### Step j

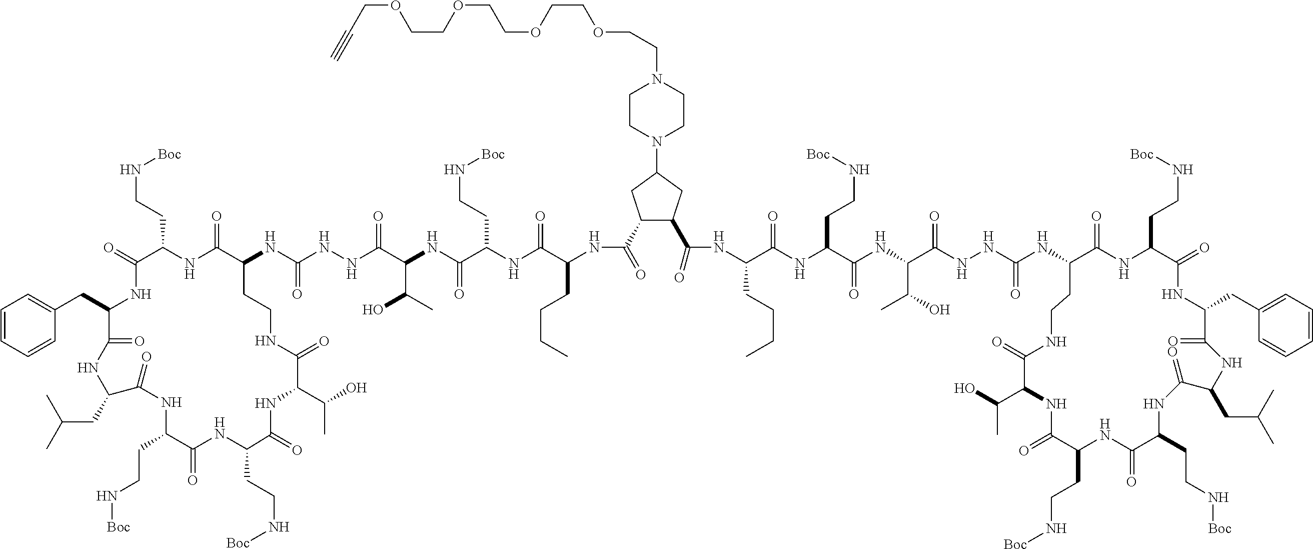

A solution of product from the previous step (1.60 g, 1.13 mmol) and diacid from step e (0.350 g, 0.512 mmol) were dissolved in DMF (15 mL) then charged with DIEA (0.536 mL, 0.513 mmol) and HATU (0.429 g, 1.13 mmol, added via syringe pump over 1 h) at room temperature while stirring. After an additional 1 h of stirring, the reaction was concentrated and purified by reversed phase liquid chromatography (RPLC) using an Isco CombiFlash liquid chromatograph eluted with 40% to 100% methanol and water, using 0.1% TFA as the modifier. Yield of product was 0.779 g, 44% yield. Ion(s) found by LCMS: [M−1(Boc)/3]+1=1130.7, [M−2(Boc)/3]+1=1097.3, [M−3(Boc)/3]+1=1064.1

### Step k

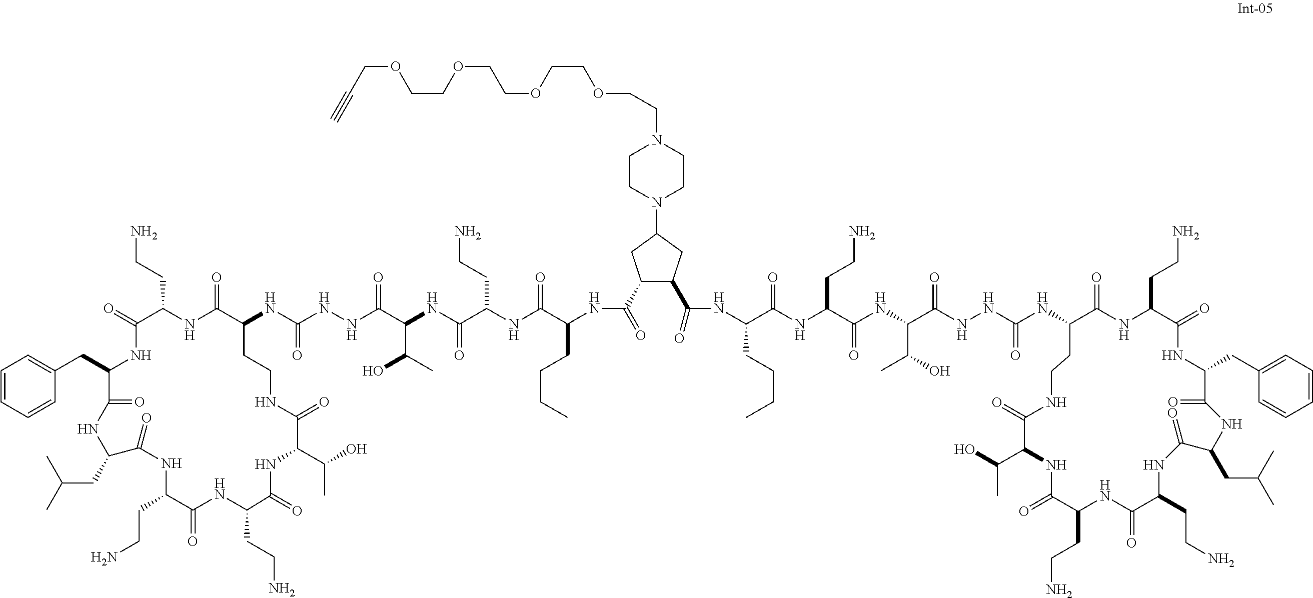

Product from the previous step (0.779 g, 0.223 mmol) was dissolved in TFA (5 mL) and stirred for 5 min then stripped of TFA and purified by reversed phase liquid chromatography (RPLC) using an Isco CombiFlash liquid chromatograph eluted with 5% to 100% methanol and water, using 0.1% TFA as the modifier. Yield of product was 0.341 g, 40% yield. Ion(s) found by LCMS: [(M+3H)/3]⁺=897.0, [(M+4H)/4]⁺=673.1, [(M+5H)/5]⁺=538.7.

### Example 206—Synthesis of Int-107

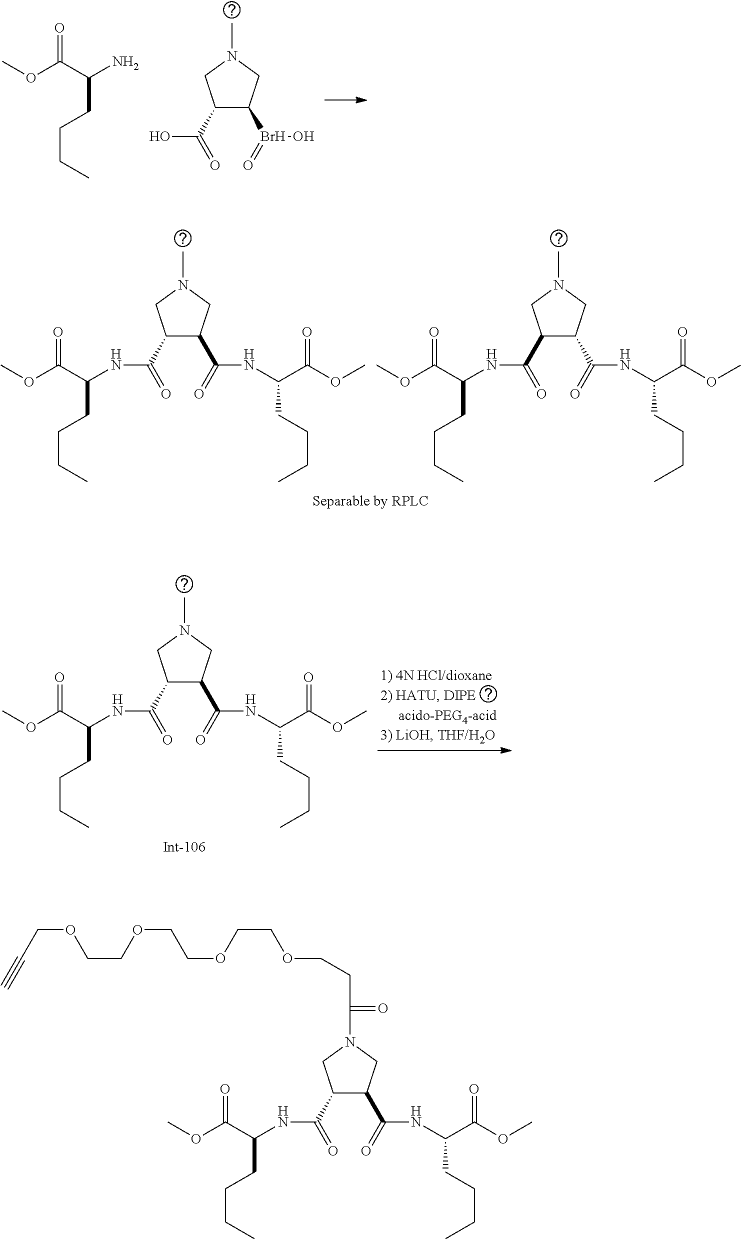

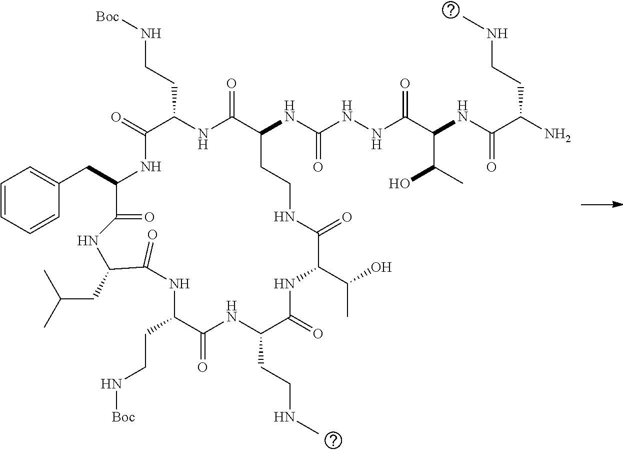

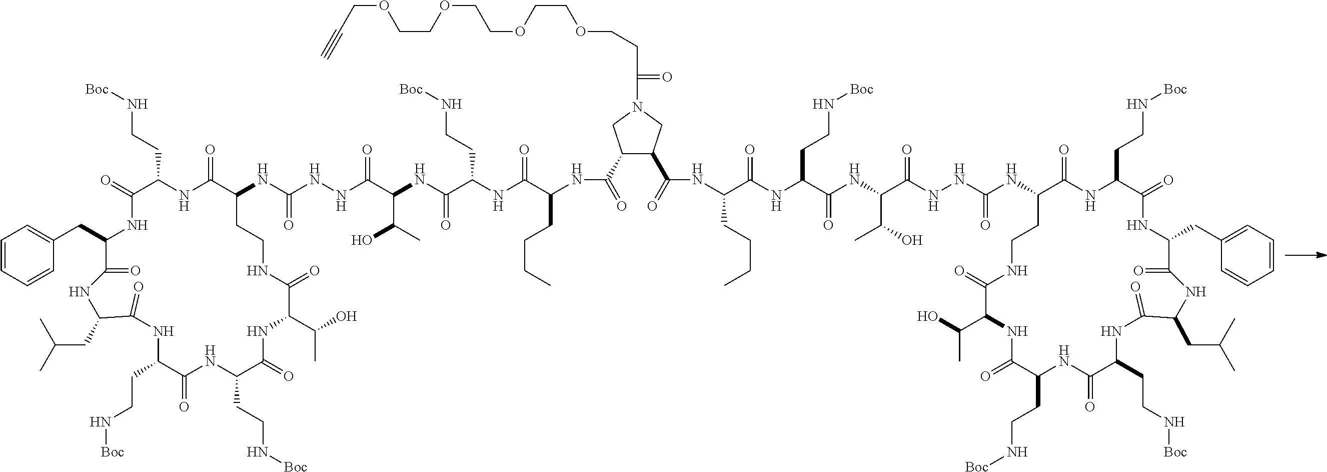

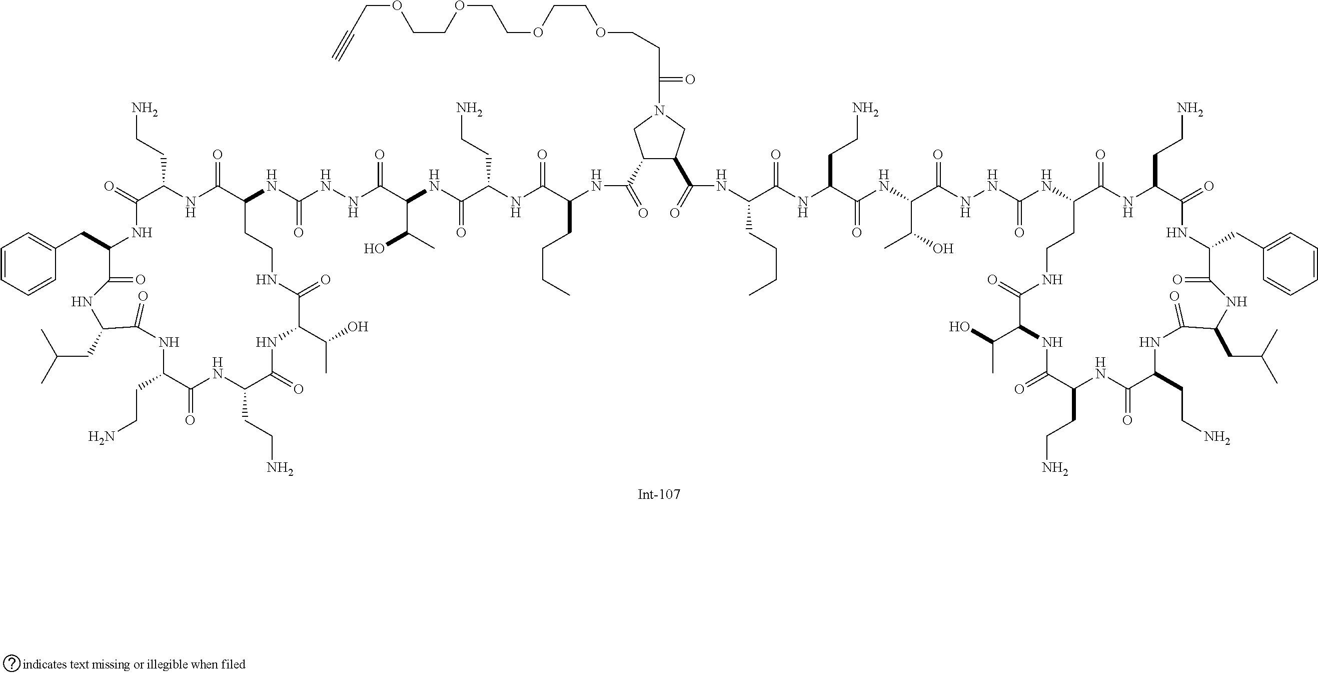

### Step a

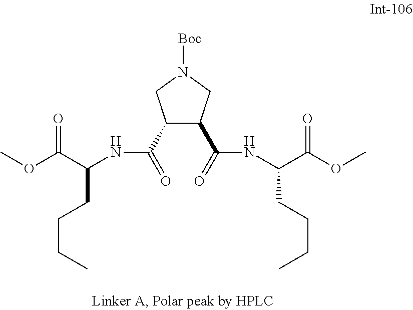

A solution of L-norleucine methyl ester HCl salt (10.014 g, 77.14 mmol), racemic-trans-1-(tert-butoxycarbonyl)pyrrolidine-3,4-dicarboxylic acid (10.000 g, 38.57 mmol), EDCI (18.485 g, 98.43 mmol), HOAt (13.125 g, 98.43 mmol), and sodium bicarbonate (12.962 g, 154.3 mmol) dissolved in dichloromethane/DMF (5:1, 120 mL) was stirred in room temperature for 4 h. The reaction was concentrated and the residue was precipitated in 1.0 N aq. HCl (200 mL, while stirring rapidly). The solid was collected and purified by reversed phase liquid chromatography (RPLC) using an Isco CombiFlash liquid chromatograph eluted with 40% to 60% acetonitrile and water. Two products, Linker A polar by re HPLC and Linker B hydrophobic by re HPLC, were isolated. Yield of Linker A is 8.905 g, 45%, theoretic yield 50%). Ions were found by LCMS (8-minute, 10-90%): Rt=3.36 min., positive mass:(M−Boc+H)⁺=414.2, negative mass: (M−H⁺)=512.2.

### Step b

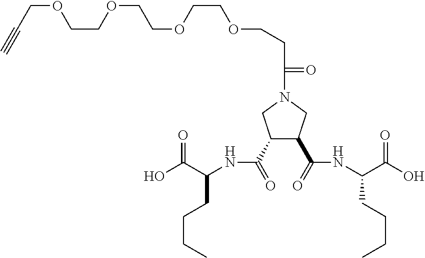 [SMILES: C#CCOCCOCCOCCOCCC(=O)N1C[C@@H](C(=O)N[C@@H](CCCC)C(=O)O)[C@H](C(=O)N[C@@H](CCCC)C(=O)O)C1]

Int-106 (2.000 g, 3.894 mmol) was treated with 4N HCl/dioxane for 1 h. The white HCl salt precipitates as it forms. After removal of excess HCl/dioxane the white solid was dissolved in DMF (30 mL) followed by adding diisopropylethylamine. Propargyl-PEG4-acid (1.115 g, 4.283 mmol) was dissolved in DMF (12 mL) and was added to a stirring mixture of Linker A and DIPEA DMF solution via syringe pump at rate of 3 mL/hr. The mixture was stirred for another 30 minutes and the solvent was removed. Then the residue was applied to reversed phase liquid chromatography (RPLC) using an Isco Combiflash liquid chromatograph eluted with 30% to 70% acetonitrile and water. The solvent was removed by rotovap to give the di-methyl ester as a viscous oil (2.000 g, 78%). Ions found by LCMS (5-minute run, 40-90%): Rt=2.36 min., (M+H)+=656.3.

The dimethyl ester was hydrolyzed stirring in a 1/1 mixture of THF/water (40 mL) containing LiOH (0.1581 g, 6.405 mmol) for 30 minutes. The mixture was adjusted to pH 5 with Amberlite IRN-77 resin. The solution was filtrated and washed with methanol, then concentrated and dried (1.914 g, 100%) without purification for next step reaction. Ion was found by LCMS (5 minute run, 10-90%): Rt=2.86 min., (M+H)⁺=628.3.

### Step c

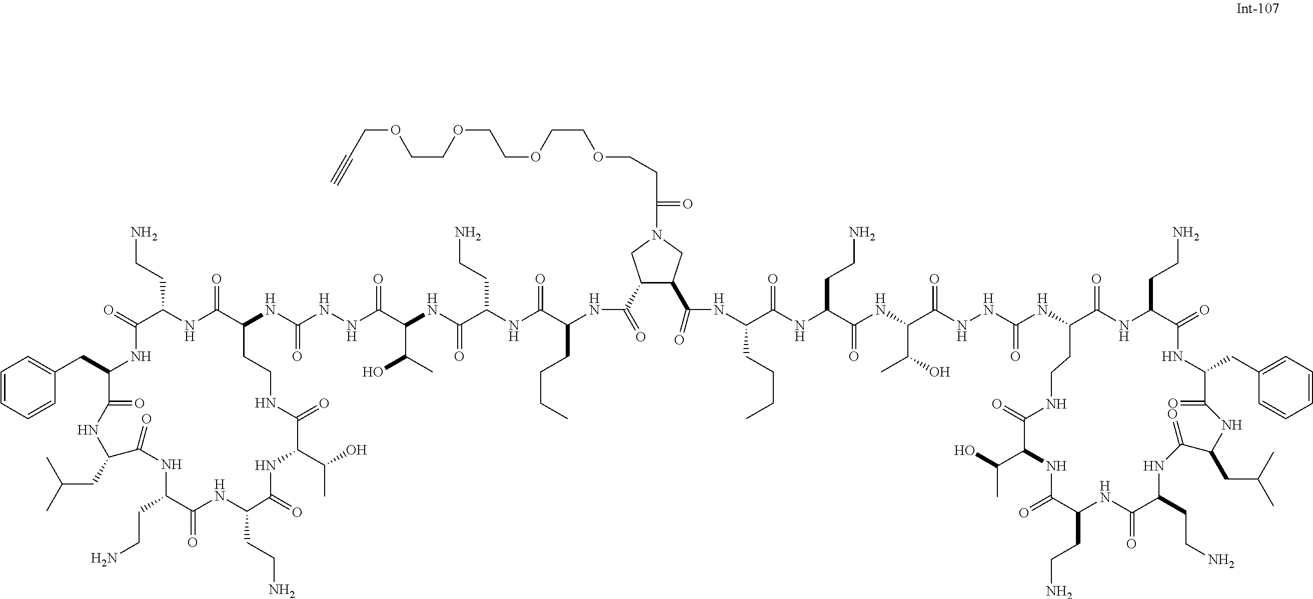

Aza-Gly-PMBD-NH₂ (2.840 g, 1.998 mmol, described in Example 205, step a), central linker diacid (0.6270 g, 0.9989 mmol), EDCI (0.7787 g, 2.497 mmol), HOAt (0.3399 g, 2.497 mmol) and DIPEA (0.70 mL, 4.0 mmol) were mixed. The reaction solution was stirred overnight. The reaction was concentrated, then triturated with EtOH (˜30 mL). Sonication, filtration and EtOH wash were used to purify the product followed by drying to give 2.224 g as a white solid (65%). Ion found by LCMS (7-minute run, 40-95%): Rt=2.40 min, (M−3 Boc+3H⁺)/3=1045.4. The white solid was treated with 30 mL of TFA for 30 min followed by removal of excess TFA. The residue was applied to reversed phase liquid chromatography (RPLC) using an Isco Combiflash liquid chromatograph eluted with 55% to 45% acetonitrile and water using 0.1% TFA modifier to give the desired product as a TFA salt. Yield: 90%. Ion(s) found by LCMS (7-minute, 40-95%): Rt=2.40 min., positive high molecular weight: (M+3H⁺)/3=878.8, (M+4H⁺)/4=659.4, (M+5H⁺)/5=527.6. (M+6H⁺)/6=439.9, (M+7H⁺)/7=377.2.

### Example 207—Synthesis of Int-109

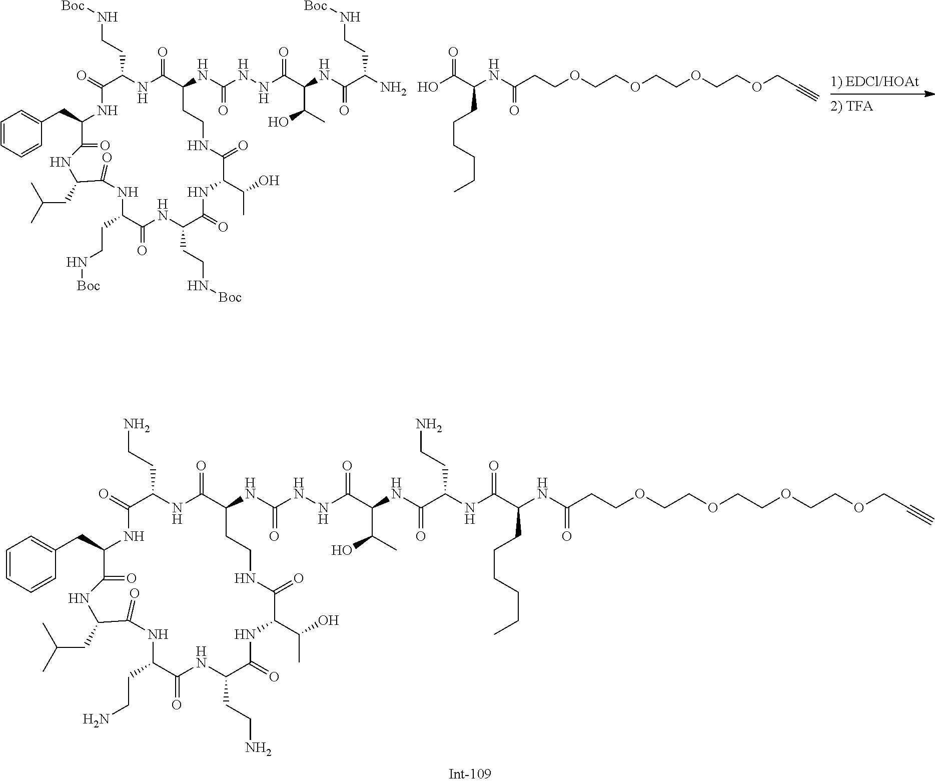 [SMILES: CC(C)C[C@@H]1NC(=O)[C@@H](Cc2ccccc2)NC(=O)[C@H](CCNC(=O)OC(C)(C)C)NC(=O)[C@@H](NC(=O)NNC(=O)[C@@H](NC(=O)[C@@H](N)CCNC(=O)OC(C)(C)C)[C@@H](C)O)CCNC(=O)[C@H]([C@@H](C)O)NC(=O)[C@H](CCNC(=O)OC(C)(C)C)NC(=O)[C@H](CCNC(=O)OC(C)(C)C)NC1=O ; C#CCOCCOCCOCCOCCC(=O)N[C@@H](CCCCCC)C(=O)O ; C#CCOCCOCCOCCOCCC(=O)N[C@@H](CCCCCC)C(=O)N[C@@H](CCN)C(=O)N[C@H](C(=O)NNC(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O]

Tetra-Boc-Aza-Gly-PMBD-NH₂ (0.3541 g, 0.2491 mmol, described in Example 205), propargyl-PEG4-octanoyl-2-amino acid (0.1100 g, 0.2740 mmol, Example 194), EDCI (0.0716 g, 0.374 mmol), HOAt (0.0509 g, 0.374 mmol) and DIPEA (0.087 mL, 0.50 mmol) were mixed in DMF (3 mL). The reaction solution was stirred for overnight, then poured into rapidly stirring 1N aq. HCl (50 mL) to precipitate the crude product. The dried crude solid was dissolved in minimum amount of NMP and purified by reversed phase liquid chromatography (RPLC) using an Isco CombiFlash liquid chromatograph eluted with 45% to 85% methanol and water using 0.1% TFA modifier to give 0.223 g, 50% yield. Ions found by LCMS (7-minute run, 40-95%): Rt=3.27 min. (M−2 Boc+2H⁺)/2=803.2.

The Boc-protected product was treated with 5 mL of TFA for 30 min followed by removal of excess TFA. The residue purified by reversed phase liquid chromatography (RPLC) using an Isco CombiFlash liquid chromatograph eluted with 5% to 45% acetonitrile and water using 0.1% TFA modifier to give the desired product as TFA salt powder after lyophilization. Yield: 70%. Ion(s) found by LCMS (5-minute run, 10-95%): Rt=1.10 min. (M+2H⁺)/2=703.4, (M+3H⁺)/3=469.0, (M+4H⁺)/4=352.0.

### Example 208—Synthesis of Int-110

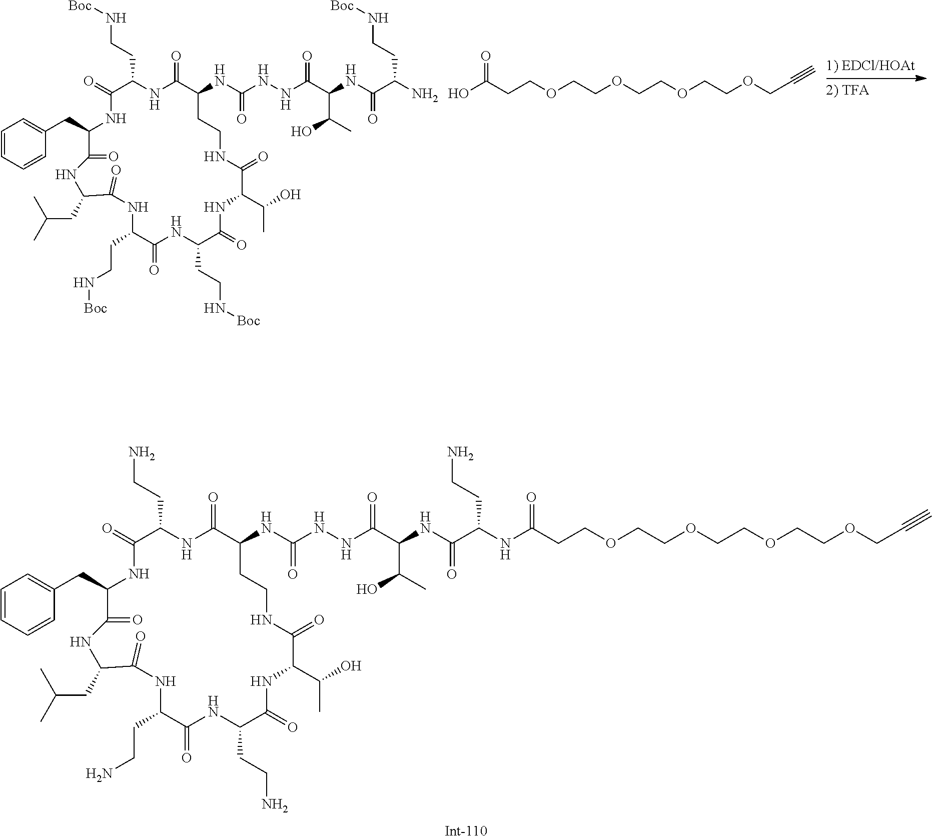 [SMILES: CC(C)C[C@@H]1NC(=O)[C@@H](Cc2ccccc2)NC(=O)[C@H](CCNC(=O)OC(C)(C)C)NC(=O)[C@@H](NC(=O)NNC(=O)[C@@H](NC(=O)[C@@H](N)CCNC(=O)OC(C)(C)C)[C@@H](C)O)CCNC(=O)[C@H]([C@@H](C)O)NC(=O)[C@H](CCNC(=O)OC(C)(C)C)NC(=O)[C@H](CCNC(=O)OC(C)(C)C)NC1=O ; C#CCOCCOCCOCCOCCC(=O)O ; C#CCOCCOCCOCCOCCC(=O)N[C@@H](CCN)C(=O)N[C@H](C(=O)NNC(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O]

Aza-gly-PMBD-NH₂ (0.3277 g, 0.2305 mmol, described in Example 205), propargyl-PEG4 acid (0.0660 g, 0.254 mmol), EDCI (0.0663 g, 0.346 mmol), HOAt (0.0471 g, 0.346 mmol) and DIPEA (0.080 mL, 0.46 mmol) were mixed in DMF (4 mL). The reaction solution was stirred for overnight then poured into 1N aq. HCl (50 mL) to precipitate the crude product. After filtration, the dried crude solid was dissolved in minimum amount of NMP and purified by reversed phase liquid chromatography (RPLC) using an Isco CombiFlash liquid chromatograph eluted with 45% to 85% methanol and water using 0.1% TFA modifier to give 0.173 g, 45% yield, white powder product. Ion(s) found by LCMS (7-minute run, 60-95%): Rt=0.86 min., (M−2 Boc+2H⁺)/2=732.6.

The white solid was treated with 5 mL of TFA for 30 min followed by removal of excess TFA. The residue was purified by reversed phase liquid chromatography (RPLC) using an Isco CombiFlash liquid chromatograph eluted with 5% to 45% acetonitrile and water using 0.1% TFA modifier to give the desired product as TFA salt powder after lyophilization. Yield: 70%. Ion(s) found by LCMS (5-minute run, 10-95%): Rt=1.0 min, (M+2H⁺)/2=632.4. (M+3H⁺)/3=422.0, (M+4H⁺)/4=316.8.

### Example 209—Synthesis of Int-111

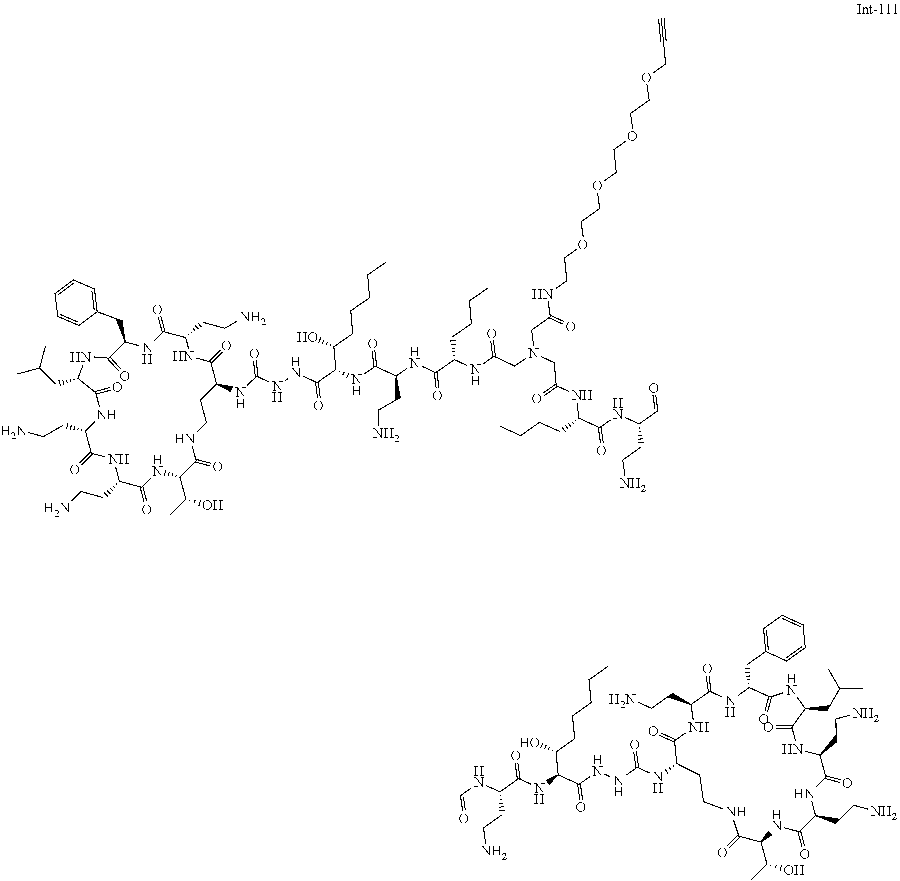

The title compound was prepared analogously to Example 199, where the C3-extended threonine was replaced with C4-extended threonine. Synthesis of extended threonine derivatives is described in Example 177. Ion(s) found by LCMS: [(M+5H)/5]⁺=550.5, [(M+6H)/6]⁺=458.9, [(M+7H)/7]⁺=393.5.

### Example 210—Synthesis of Int-112

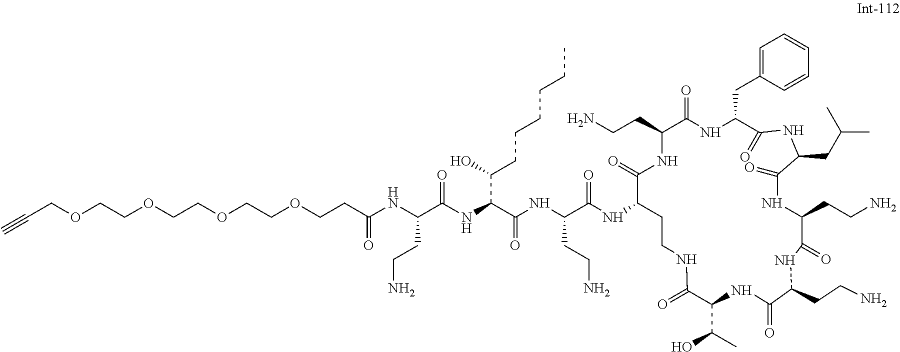

The title compound is prepared analogously to Example 195, where the C5 extended threonine is replaced with C6 extended threonine.

### Example 211—Synthesis of Int-113

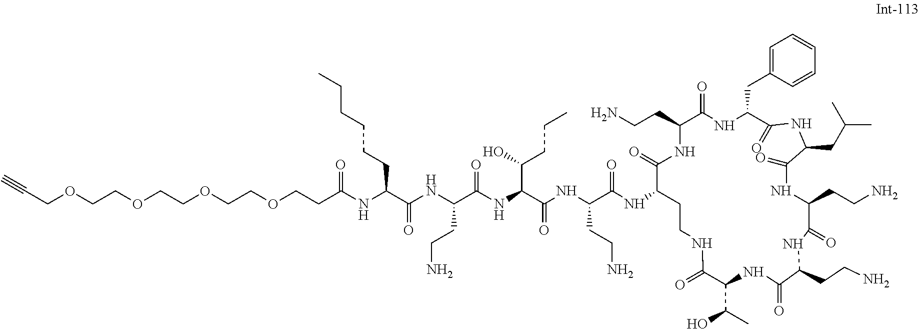

The title compound is prepared analogously to Example 188, where the dimethyl-extended threonine is replaced with a C3-extended threonine.

### Example 212—Synthesis of Int-114 (N-Methyl-PMB-Heptapeptide Intermediate)

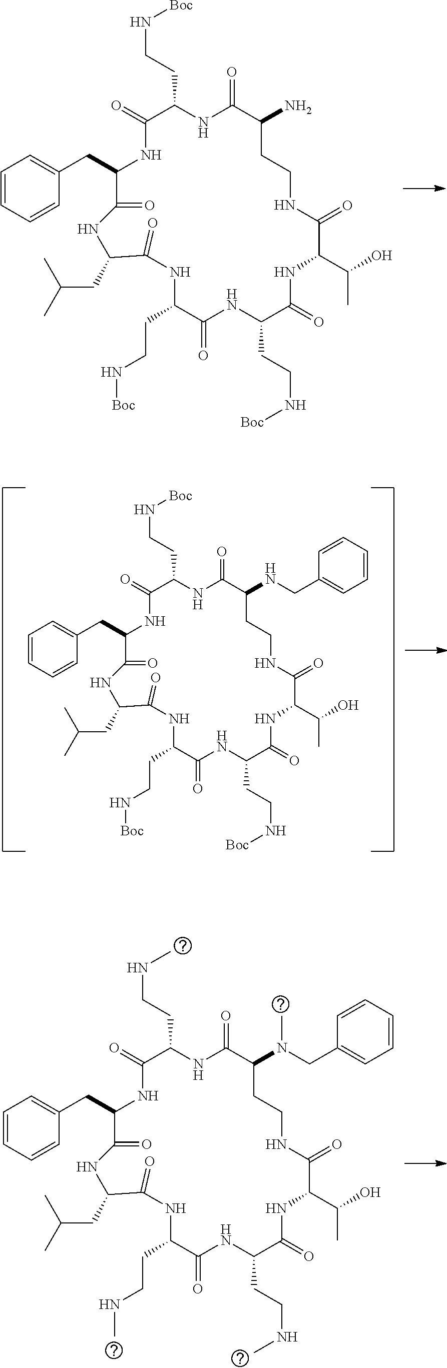

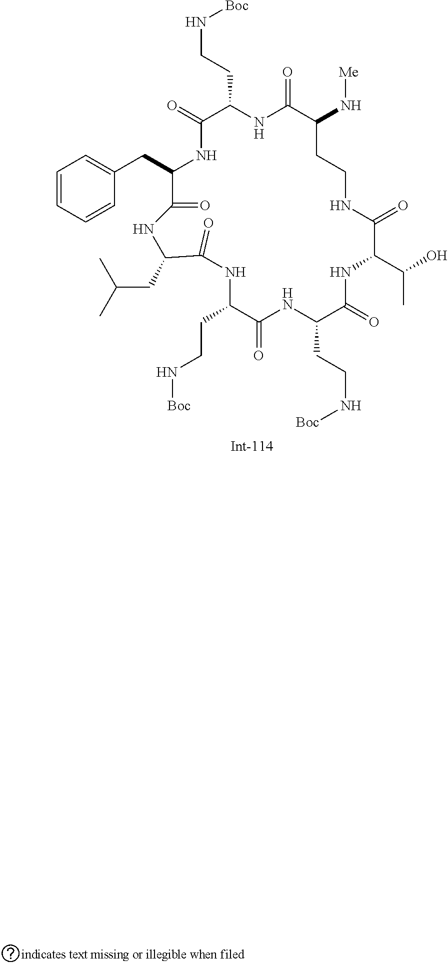

### Step a

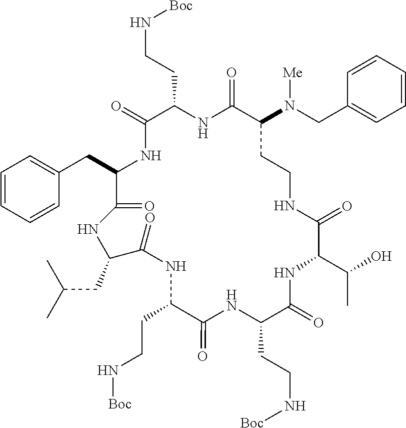

Dissolve PMBH (10 g, 9.42 mmol, described in Example 2) in methanol (100 mL), add a few drops of glacial acetic acid (to pH-5-6), then add benzaldehyde (1.2 g, 11.3 mmol) and let stir for 15 minutes. Cool to 0*C, ice/water bath and add half (1.5 eq, 921 mg, 14.68 mmol) of sodium cyanoborohydride in 3 portions over 10 minutes. Remove ice bath and let stir-12 hours (overnight). Check by LC/MS to insure complete conversion to the mono benzylated PMBH intermediate. Cool to 0*C and add formaldehyde (7 eq, 5 mL, 67.7 mmol) followed by the addition of sodium cyanoborohydride (1 eq, 606 mg, 9.64 mmol) in 3 portions over 10 minutes, let stir at 0*C for 20 minutes and remove ice bath. Let reaction stir for 2 hours then add additional 3 eq of formaldehyde (2.1 mL, 29.0 mmol) and sodium cyanoborohydride (320 mg, 5.1 mmol) stir for 6 more hours. Dilute with saturated sodium bicarb (pH˜8) and remove approx half the methanol by rotovap. Dilute with ethyl acetate and saturated sodium bicarb and extract into ethyl acetate (3×). Dry the combined organic extracts over sodium sulfate, filter and concentrate. Purify by normal phase silica gel chromatography (0-5% methanol in dcm containing 1% triethylamine, 35 minute gradient). The product elutes at ˜3.3% methanol. 8.2 grams (74%) of the product was isolated as a white solid. LC/MS [M-Boc+2H+]=533.8.

### Step b

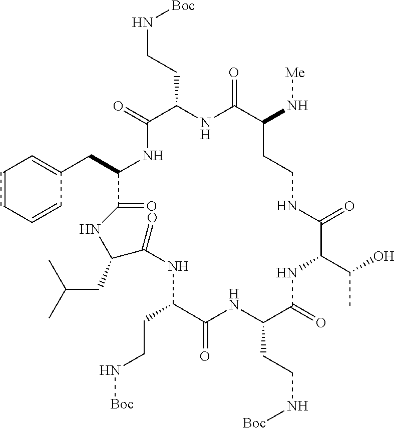

8.2 grams of the N-methyl, benzyl PMBH(trisBoc) intermediate was dissolved in 100 mL of methanol in a 250 round bottom flask. 20% Pd(OH)₂ (2 g) was added followed by a few drops of glacial acetic acid (to pH-6). The flask was evacuated and flushed with hydrogen gas (3×) and then the mixture was stirred under 1 atm of hydrogen gas for 12 hours. The reaction was monitored by LC/MS and HPLC to insure complete removal of the benzyl group. Upon completion the mixture was filtered through celite and the pH was adjusted to ˜7 with a few drops of triethylamine. The solvent was removed by rotovap and the residue was purified by normal phase silica gel chromatography (0-7% methanol in dcm containing 1% triethylamine, 35 minute gradient). The product elutes at ˜4.2% methanol. 6.65 grams (87%) of the product was isolated as a white solid. LC/MS [M-Boc+2H+]=488.8.

### Example 213—Preparation of PEG4-Azido hIgG1 Fc

PEG4-azido-NHS ester (98% pure, 180 μmol, 9.5 equivalents, 71.4 mg in 0.5 mL of DMF and diluted to 3.60 mL with pH 7.4 PBS 1× buffer solution) was added to a solution of hIgG1 Fc (SEQ ID NO: 4) (1103 mg in 70.0 mL of pH 7.4 PBS, MW-58,000 Da, 19.0 μmol) and the mixture was shaken gently for 12 hours at ambient temperature. The solution was concentrated using a centrifugal concentrator (30,000 MWCO) to a volume of ˜1.5 mL. The crude mixture was diluted 1:10 in PBS pH 7.4, and concentrated again. This wash procedure was repeated for total of three times. The small molecule reagent was removed with this wash procedure. The concentrated Fc-PEG4-azide was diluted to 70.0 mL with pH 7.4 PBS 1× buffer and ready for Click conjugation. The purified material was quantified using a Nanodrop™ UV visible spectrophotometer (using a calculated extinction coefficient based on the amino acid sequence of h-IgG1). Yield is quantitative after purification. DAR=4.3 determined by MALDI. Other DAR values can be obtained by adjusting the number of equivalents of PEG4-azido NHS-ester.

### Example 214—Purification of Fc Conjugates

The crude solutions were diluted with pH 7.4 PBS to a final concentration of 1 mg/mL, and ultrafiltered (10,000 MWCO) to a volume of 1 mL, two times. The crude mixtures were then diluted 1:10 in PBS pH 7.4, and purified using MabSelect Sure Resin (GE Healthcare, Chicago, Ill., USA), followed by size exclusion chromatography. Purified material was quantified using a Nanodrop™ UV visible spectrophotometer (using a calculated extinction coefficient based on the amino acid sequence of the Fc used in the conjugation), and concentrated to approximately 10 mg/mL using a centrifugal concentrator (10,000 MWCO). Purified molecules were analyzed using 4-12% Bis Tris SDS PAGE gels by loading 1-2 μg of each molecule into the gel, and staining using instant Blue staining. Each gel included a molecular weight ladder with the indicated molecular weight standards. Yields are typically 40-60%. MALDI MS analysis was used to determine a final average DAR.

### Example 215—Synthesis of Conjugate 80

All solutions were prepared in buffer PBS 7.4. Alkyne derivatized small molecule Int-78 (30.6 mg, 16.7 μmol, described in Example 178) was dissolved in a solution of azido functionalized hIgG1 Fc (30 mg, 0.51 μmole, 1.67 ml, described in Example 213). The mixture was treated with 0.2 M solution of L-ascorbic acid sodium (20.4 μmole, 102 μl) followed by 0.1 M solution of THPTA (10.2 μmol, 0.1 M solution of CuSO₄ (10.2 μmol, 102 μl) and then gently shaken overnight. Maldi TOF analysis of the purified final product gave an average mass of 61,842 Da (DAR=1.9). Yield 14.03 mg, 47% yield.

### Example 216—Synthesis of Conjugate 81

A solution of azido functionalized hIgG1 Fc (100 mg, 1.28 μmol, 6.43 mL, MW=58219 Da. DAR=6) were added alkyne derivatized small molecule Int-80 (Example 180 TFA salt, 106 mg, 0.052 mmol) and then freshly prepared pH 7.4 PBS solutions of CuSO₄ (0.68 mL of 50.0 mM, 20 eq), tris(3-hydroxypropyltriazolylmethyl)-amine (THPTA, 0.68 mL of 50.0 mM, 20 eq), and sodium ascorbate (1.03 mL of 50.0 mM, 30 eq). The resulting homogeneous solution was agitated by rocker table for 12 h. The product was purified using the procedure described in Example 214. Yields are typically 40-60%. MALDI MS analysis showed a range of masses (58,000-75,000) with an average of mass of 74,912. Average DAR=9.5.

### Example 217—Synthesis of Conjugate 82

A solution of azido functionalized hIgG1 Fc (100 mg, 1.72 μmol, 8.9 mL, MW=58219 Da, DAR=6.8) were added alkyne derivatized small molecule Int-84 (Example 184 TFA salt, 71 mg, 0.036 mmol) and then freshly prepared pH 7.4 PBS solutions of CuSO₄ (0.68 mL of 50.0 mM, 20 eq), tris(3-hydroxypropyltriazolylmethyl)-amine (THPTA, 0.68 mL of 50.0 mM, 20 eq), and sodium ascorbate (1.03 mL of 50.0 mM, 30 eq). The resulting homogeneous solution was agitated by rocker table for 12 h. The product was purified using the procedure described in Example 214. Yields are typically 40-60%. MALDI MS analysis showed a range of masses (58,000-75,000) with an average of mass of 69,204. Average DAR=6.6.

### Example 218—Synthesis of Conjugate 83

A solution of azido functionalized hIgG1 Fc (100 mg, 1.72 μmol, 8.9 mL, MW=58219 Da, DAR=6.8) were added alkyne derivatized small molecule Int-83 (Example 183 TFA salt, 71 mg, 0.036 mmol) and then freshly prepared pH 7.4 PBS solutions of CuSO₄ (0.68 mL of 50.0 mM, 20 eq), tris(3-hydroxypropyltriazolylmethyl)-amine (THPTA, 0.68 mL of 50.0 mM, 20 eq), and sodium ascorbate (1.03 mL of 50.0 mM, 30 eq). The resulting homogeneous solution was agitated by rocker table for 12 h. The product was purified using the procedure described in Example 214. Yields are typically 40-60%. MALDI MS analysis showed a range of masses (58,000-75,000) with an average of mass of 69,014. Average DAR=6.8.

### Example 219—Synthesis of Conjugate 84

A solution of azido functionalized hIgG1 Fc (80 mg, 1.37 μmol, 7.11 mL, MW=58219 Da. DAR=6.8) were added alkyne derivatized small molecule Int-87 (Example 187 TFA salt, 59 mg, 0.029 mmol) and then freshly prepared pH 7.4 PBS solutions of CuSO₄ (0.55 mL of 50.0 mM, 20 eq), tris(3-hydroxypropyltriazolylmethyl)-amine (THPTA, 0.55 mL of 50.0 mM, 20 eq), and sodium ascorbate (0.82 mL of 50.0 mM, 30 eq). The resulting homogeneous solution was agitated by rocker table for 12 h. The product was purified using the procedure described in Example 214. Yields are typically 40-60%. MALDI MS analysis showed a range of masses (58,000-75,000) with an average of mass of 68,284. Average DAR=5.6.

### Example 220—Synthesis of Conjugate 86

A solution of azido functionalized hIgG1 Fc (100 mg, 8.9 ml, 1.7182 μmol, Example 213) was added into a 15 ml plastic tube containing alkyne derivatized small molecule Int-92 (Example 192 TFA salt 64.3 mg, 36.08 μmol). After the mixture was gently shaken to dissolve all solids, it was treated with a solution of L-ascorbic acid sodium salt (59.4 mg, 0.3 mmol), copper (II) sulfate (15.9 mg, 0.1 mmol), and THPTA (43.5 mg, 0.1 mmol) in PBS 7.4 buffer. The resulting mixture was gently shaken overnight, then purified by affinity chromatography over a protein A column, followed size exclusion chromatography (See Example 214 for purification details). Maldi TOF analysis of the purified final product gave an average mass of 66,664. Da (DAR=5). Yield 55.35 mg, 55% yield.

### Example 221—Synthesis of Conjugate 86

A solution of azido functionalized hIgG1 Fc (100 mg, 1.37 μmol, 7.11 mL, MW=58219 Da, DAR=6.8) were added alkyne derivatized small molecule Int-94 (Example 194 TFA salt, 56 mg, 0.0275 mmol) and then freshly prepared pH 7.4 PBS solutions of CuSO₄ (0.55 mL of 50.0 mM, 20 eq), tris(3-hydroxypropyltriazolylmethyl)-amine (THPTA, 0.55 mL of 50.0 mM, 20 eq), and sodium ascorbate (0.82 mL of 50.0 mM, 30 eq). The resulting homogeneous solution was agitated by rocker table for 12 h. The product was purified using the procedure described in Example 214. Yields are typically 40-60%. MALDI MS analysis showed a range of masses (58,000-75,000) with an average of mass of 69,708. Average DAR=6.6.

### Example 222—Synthesis of Conjugate 87

A solution of azido functionalized hIgG1 Fc (100 mg, 1.72 μmol, 5.61 mL, MW=58219 Da, DAR=4.7) were added alkyne derivatized small molecule Int-100 (Example 200 TFA salt, 65 mg, 0.034 mmol) and then freshly prepared pH 7.4 PBS solutions of CuSO₄ (0.68 mL of 50.0 mM, 20 eq), tris(3-hydroxypropyltriazolylmethyl)-amine (THPTA, 0.68 mL of 50.0 mM, 20 eq), and sodium ascorbate (1.02 mL of 50.0 mM, 30 eq). The resulting homogeneous solution was agitated by rocker table for 12 h. The product was purified using the procedure described in Example 214. Yields are typically 40-60%. MALDI MS analysis showed a range of masses (58,000-75,000) with an average of mass of 68,412. Average DAR=4.3.

### Example 223—Synthesis of Conjugate 88

A solution of copper (II) sulfate (7.4 mg, 0.047 mmol), THPTA (20.2 mg, 0.047 mmol, THPTA=tris(3-hydroxypropyltriazolylmethyl)amine), and sodium ascorbate (27.7 mg, 0.140 mmol) dissolved in 1 mL pH 7.4 PBS, was added to a solution of alkyne derivatized small molecule Int-105 (Example 205 TFA salt 250 mg, 0.065 mmol, from) and azido functionalized hIgG1 Fc (181 mg, azido DAR=4.3, concentration=17.8 mg/mL pH 7.4 PBS, sequence provided in Example 213). The resulting homogeneous solution was agitated by rocker table for 12 h, then purified by as described in the general methods section (Example 214). Yield is typically 40-60%. MALDI MS analysis showed a range of masses (58000-75000) with an average of mass of 67,328. Average DAR=3.1.

### Example 224—Synthesis of Conjugate 89

Alkyne functionalized small molecule Int-107 (1.21 g, 0.341 mol, described in Example 206) was added to hIgG1 Fc-PEG4-azide solution (1.10 g, 70.0 mL in pH 7.4 PBS 1× buffer, 19.0 mmol, described in Example 213) in a 250-mL bottle followed by adding 0.050 M CuSO₄ pH 7.4 PBS 1× buffer solution (7.58 mL, 0.380 mol), 0.050 M sodium ascorbate pH 7.4 PBS 1× buffer solution (11.37 mL, 0.570 mol), and 0.050 M THPTA pH 7.4 PBS 1× buffer solution (7.58 mL, 0.380 mol). The reaction mixture was gently shaken at room temperature overnight. The product was purified using the procedure described in the general methods section (Example 214). Yields are typically 40-60%.

### Example 225—Synthesis of Int-115

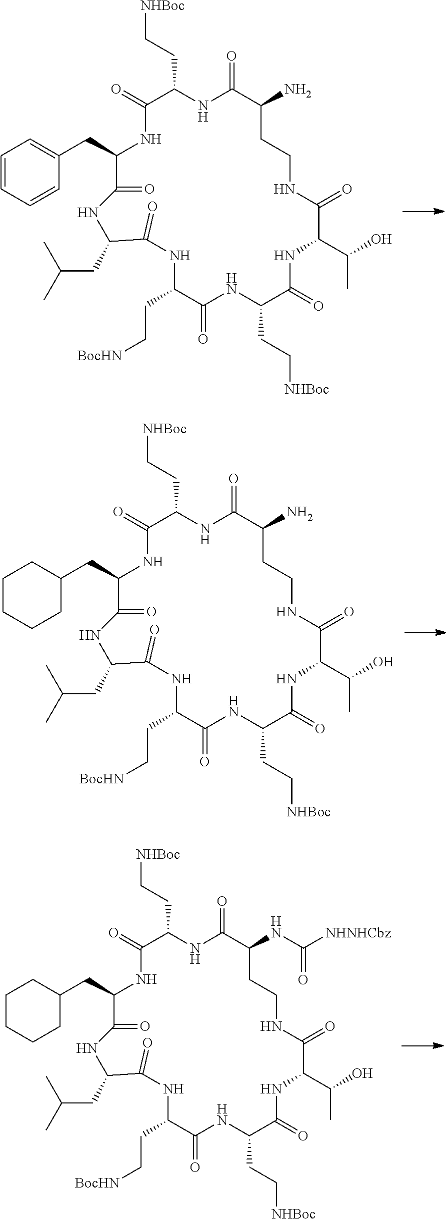

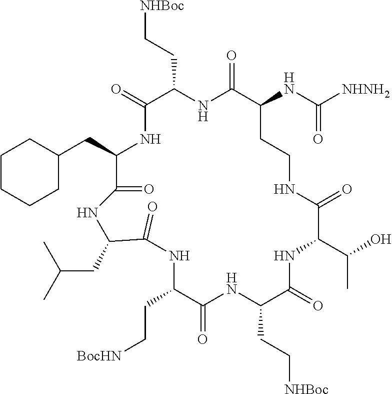 [SMILES: CC(C)C[C@@H]1NC(=O)[C@@H](CC2CCCCC2)NC(=O)[C@H](CCNC(=O)OC(C)(C)C)NC(=O)[C@@H](NC(=O)NN)CCNC(=O)[C@H]([C@@H](C)O)NC(=O)[C@H](CCNC(=O)OC(C)(C)C)NC(=O)[C@H](CCNC(=O)OC(C)(C)C)NC1=O]

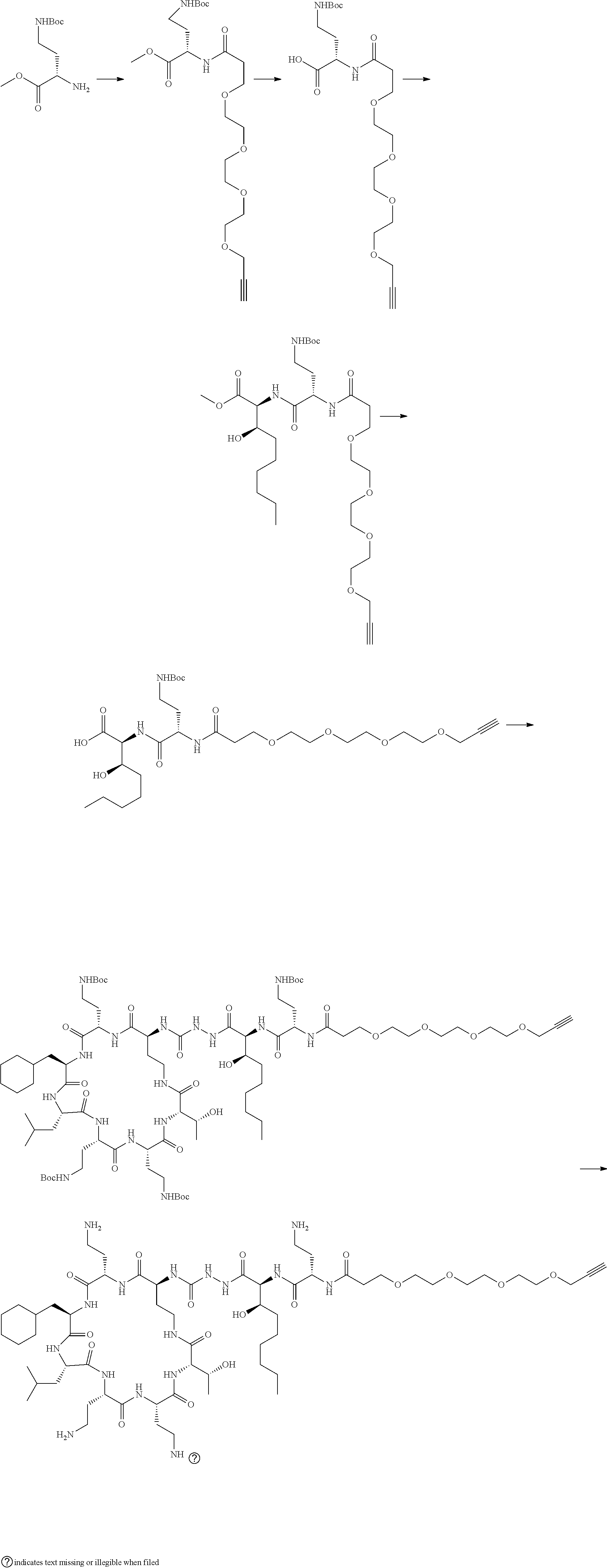

### Step a

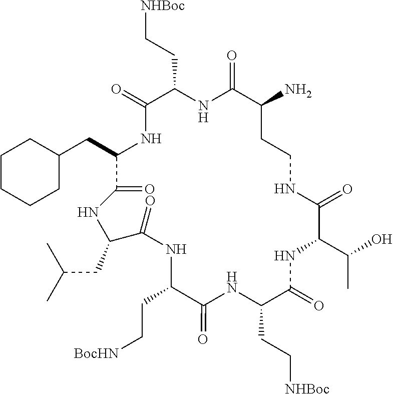

Int-1 (3.189 g, 3.0 mmol) was slowly added into acetic acid (15 mL). After all starting material was dissolved, PtO₂ (1.135 g, 5.0 mmol) was added and the resulting mixture was stirred under hydrogen for one day. The hydrogen was then replaced by nitrogen, and PtO₂ was filtered off through celite. The filtrate was concentrated by rotary evaporation, and the residue was purified through RPLC (100 g, 30 to 70% acetonitrile and water). Yield 1.86 g, 58.1%. Ions found by LCMS: [(M−Boc-tBu+2H)/2]⁺=456.9, [(M−3Boc+2H)/2]⁺=384.8.

### Step b

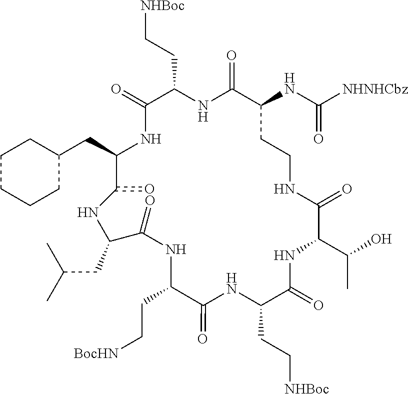

A solution of 1,1′-carbonyldiimidazole (1.12 g, 6.92 mmol) in anhydrous DMF (7 mL) was cooled in an ice-water bath and added with benzyl carbazate (1.208 g, 7.27 mmol). After stirred to dissolve all the reagent, the reaction mixture was mixed with DIPEA (260 mg, 2 mmol) and stirred at ˜4° C. to room temperature for 1 hour. The step-a product was added (1.86 g, 1.74 mmol), and the reaction was continued overnight. The mixture was then dripped into water (150 mL) containing 4N HCl in dioxane (6 mL). The precipitated product was collected by vacuum filtration and washed with water. The material was dissolved in DMF (2 mL) and purified through RPLC (100 g, 30 to 80% acetonitrile and water, using 0.1% TFA as modifier). Yield 1.47 g, 66.9%. Ions found by LCMS: [(M−2Boc+2H)/2]⁺=530.9. [(M−3Boc+2H)/2]⁺=480.9.

### Step c

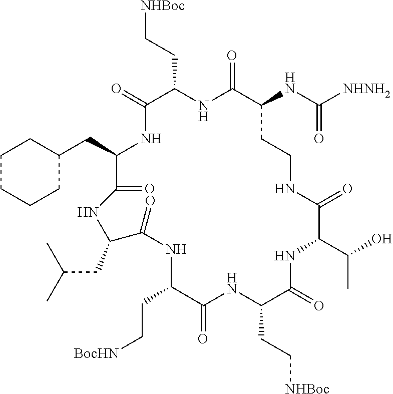

A 100 mL reaction flask was flushed with nitrogen and charged with MeOH (30 mL). Acetyl chloride (109.9 mg, 1.4 mmol) was added, followed by the step-b product (1.47 g, 1.166 mmol) and Pd/C (235 mg). The resulting mixture was stirred under hydrogen for 2 hours. Pd/C was filtered off, and the filtrate was concentrated and purified through RPLC (150 g, 30 to 65% acetonitrile and water). Yield 1.02 g, 73.3%. Ions found by LCMS: [(M−2Boc+2H)/2]⁺=463.8, [(M−2Boc-tBu+2H)/2]⁺=435.8, [(M−3Boc+2H)/2]⁺=413.8.

### Step d

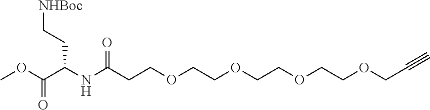 [SMILES: C#CCOCCOCCOCCOCCC(=O)N[C@@H](CCNC(=O)OC(C)(C)C)C(=O)OC]

To a solution of a mixture of propargyl-PEG4-acid (2.521 g, 9.715 mmol) and HATU (3.73 g, 9.8 mmol) in anhydrous DMF (10 mL) was added DIPEA (1.3 g, 10 mmol). After stirring for 10 minutes, the reaction mixture was added to (S)-methyl-2-amino-4-((tert-butoxycarbonyl)amino) butanoate HCl (3.23 g, 11 mmol) and additional DIPEA (1.43 g, 11 mmol). The reaction was stirred for at room temperature overnight. It was then purified by RPLC (150 g SiO2, 5% to 60% acetonitrile/water). Yield 4.3 g, 93.3%. Ions found by LCMS: [M+H]⁺=475.2, [M-Boc+H]⁺=375.2.

### Step e

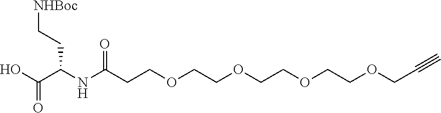 [SMILES: C#CCOCCOCCOCCOCCC(=O)N[C@@H](CCNC(=O)OC(C)(C)C)C(=O)O]

A solution of the step-d product (4.3 g, 9.06 mmol) in MeOH:THF (1:1, 20 mL) was cooled in an ice-water bath and added in portions a solution of LiOH monohydrate (571 mg, 13.6 mmol) in water (20 ml) over 45 minutes. The reaction was stirred for 1.5 more hours, then its pH was adjusted to slightly acidic by ion exchange Dowex 50W×8 hydrogen form (2.3 g). The resin was filtered, and the organic filtrate was concentrated by rotary evaporation at room temperature. Yield 4.17 g, 99.9%. Ions found by LCMS: [M+H]⁺=461.2, [M-Boc+H]⁺=361.2.

### Step f

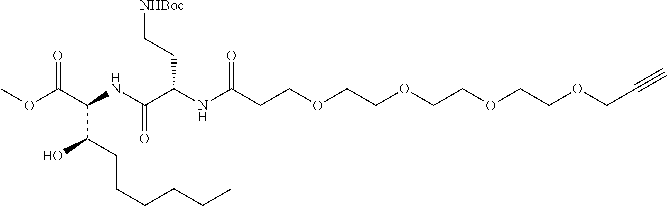

To a solution of a mixture of the step-e product (1.58 g, 3.44 mmol) and HATU (1.33 g, 3.5 mmol) in anhydrous DMF (4 mL) was added DIPEA (455 mg, 3.5 mmol). After stirred for 10 minutes, the reaction mixture was treated with (2S,3R)-2-amino-3-hydroxynonanoic acid methyl ester (1.01 g, 4.2 mmol) and an additional DIPEA (455 mg, 3.5 mmol). The reaction was stirred for 30 more minutes, then purified through RPLC (150 g SiO2, 10% to 55% acetonitrile and water). Yield 1.77 g, 79.7%. Ions found by LCMS: [M+H]⁺=646.4, [M−Boc+H]⁺=546.4.

### Step g

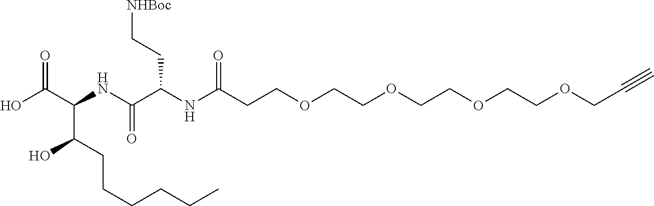 [SMILES: C#CCOCCOCCOCCOCCC(=O)N[C@@H](CCNC(=O)OC(C)(C)C)C(=O)N[C@H](C(=O)O)[C@H](O)CCCCCC]

A solution of the step-f product (1.77 g, 2.7 mmol) in MeOH:THF (1:1, 10 mL) was cooled in an ice-water bath and added in portions a solution of LiOH monohydrate (126 mg, 3 mmol) in water (5 mL) over 45 minutes. After 3 hours, it was acidified slightly by adding 4N HCl solution in dioxane (0.8 mL) and then purified by RPLC (5% to 38% acetonitrile and water). Yield 1.6 g, 96.5%. Ions found by LCMS: [M+H]⁺=632.2, [M-Boc+H]⁺=532.2.

### Step h

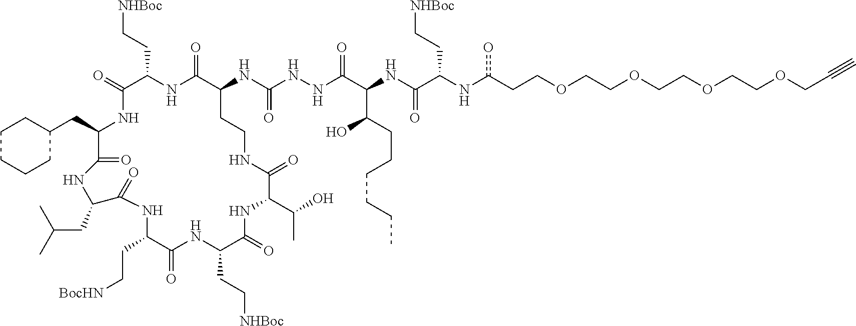

To a mixture of the step-g product (133.8 mg, 0.212 mmol) and HATU (80.5 mg, 0.212 mmol) in anhydrous DMF (1 mL) was added the step-c product (225.3 mg, 0.2 mmol) and DIPEA (39 mg, 0.3 mmol). The reaction mixture was stirred for 30 minutes and then directly purified through RPLC (50 g, 40 to 85% MeOH and water, using 0.1% TFA as modifier). Yield 300.3 mg, 86.3%. Ion found by LCMS: [(M−2Boc+2H)/2]⁺=770.6.

### Step i

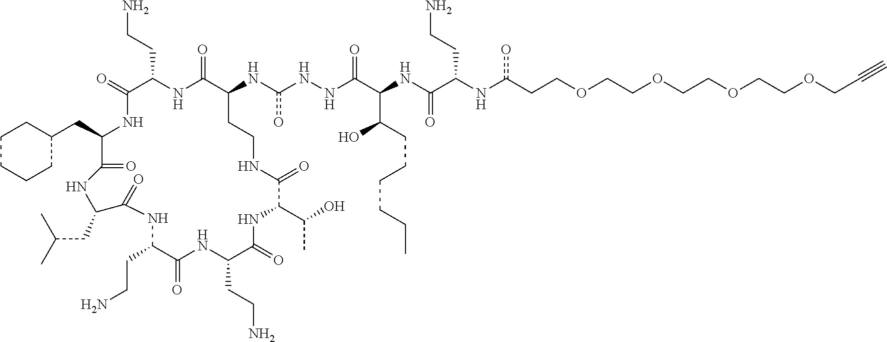

The step-h product (300.3 mg, 0.173 mmol) was dissolved in TFA (˜0.8 mL). The solution was stirred for 20 minutes, then directly purified by HPLC (5 to 35% acetonitrile and water, using 0.1% TFA as the modifier). Yield 165.3 mg, 53.2%. Ions found by LCMS: [(M+2H)/2]⁺=670.5, [(M+3H)/3]⁺=447.4, [(M+4H)/4]⁺=335.8.

### Example 226—Synthesis of Int-116

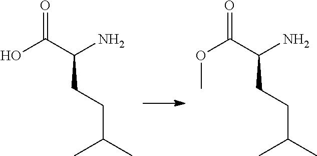

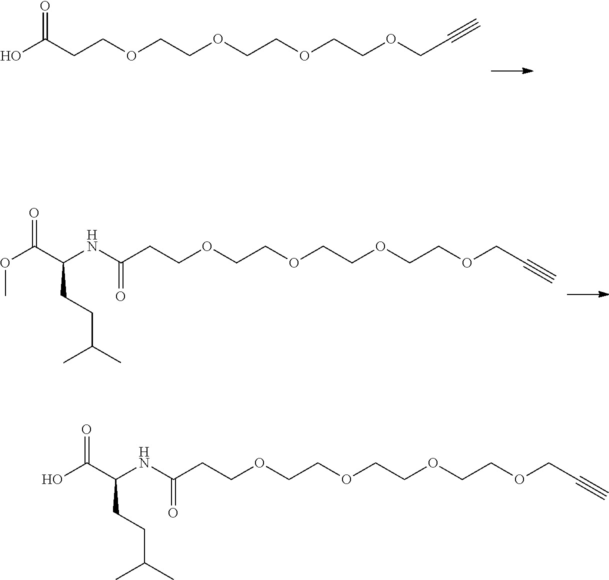 [SMILES: C#CCOCCOCCOCCOCCC(=O)O ; C#CCOCCOCCOCCOCCC(=O)N[C@@H](CCC(C)C)C(=O)OC ; C#CCOCCOCCOCCOCCC(=O)N[C@@H](CCC(C)C)C(=O)O]

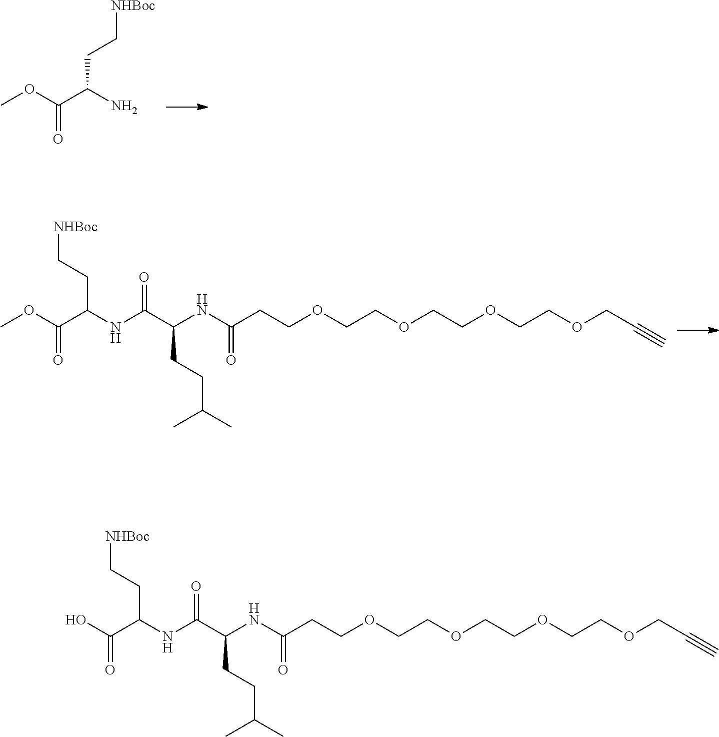 [SMILES: COC(=O)[C@@H](N)CCNC(=O)OC(C)(C)C ; C#CCOCCOCCOCCOCCC(=O)N[C@@H](CCC(C)C)C(=O)NC(CCNC(=O)OC(C)(C)C)C(=O)OC ; C#CCOCCOCCOCCOCCC(=O)N[C@@H](CCC(C)C)C(=O)NC(CCNC(=O)OC(C)(C)C)C(=O)O]

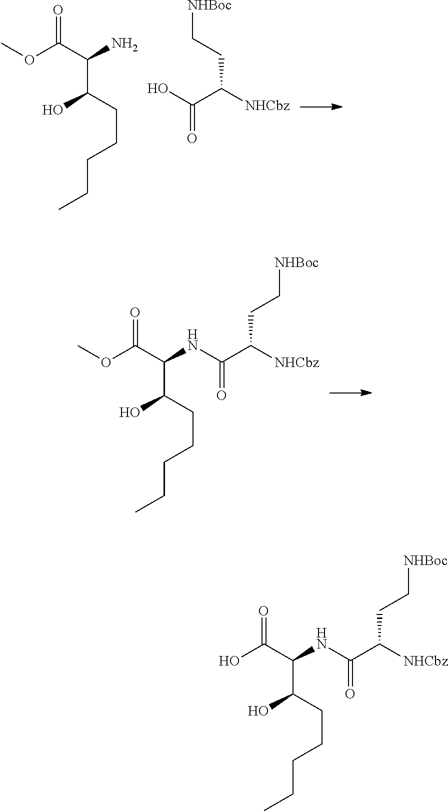 [SMILES: CCCCC[C@@H](O)[C@H](N)C(=O)OC ; CC(C)(C)OC(=O)NCC[C@H](NC(=O)OCc1ccccc1)C(=O)O ; CCCCC[C@@H](O)[C@H](NC(=O)[C@H](CCNC(=O)OC(C)(C)C)NC(=O)OCc1ccccc1)C(=O)OC ; CCCCC[C@@H](O)[C@H](NC(=O)[C@H](CCNC(=O)OC(C)(C)C)NC(=O)OCc1ccccc1)C(=O)O]

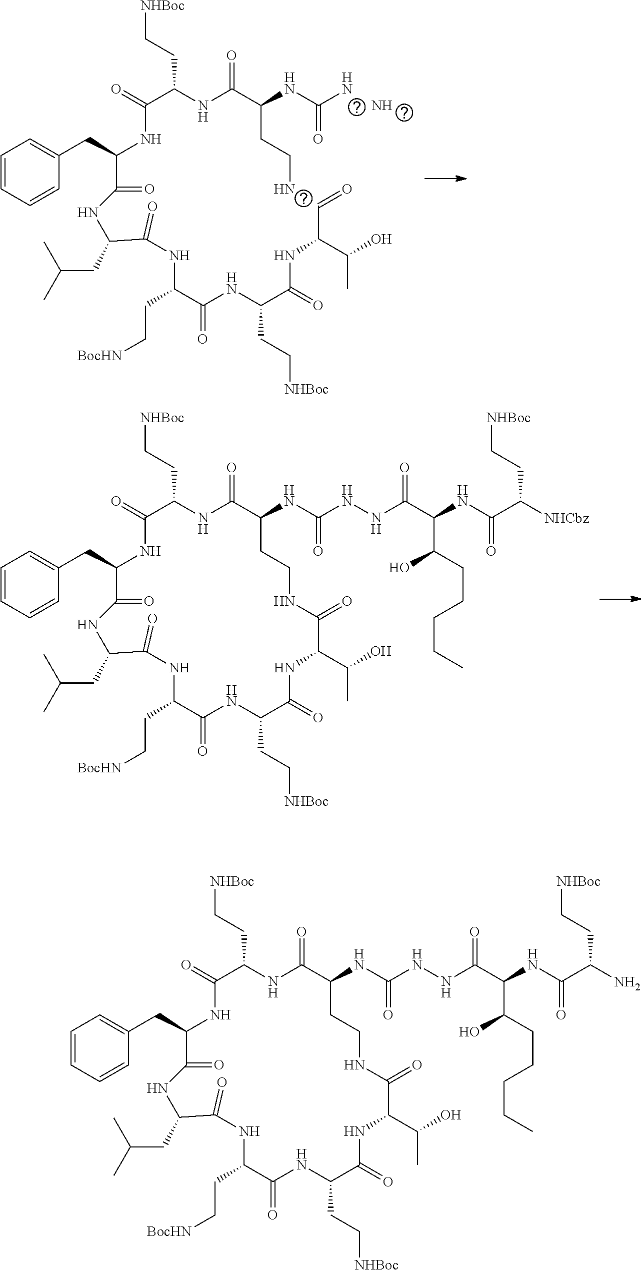

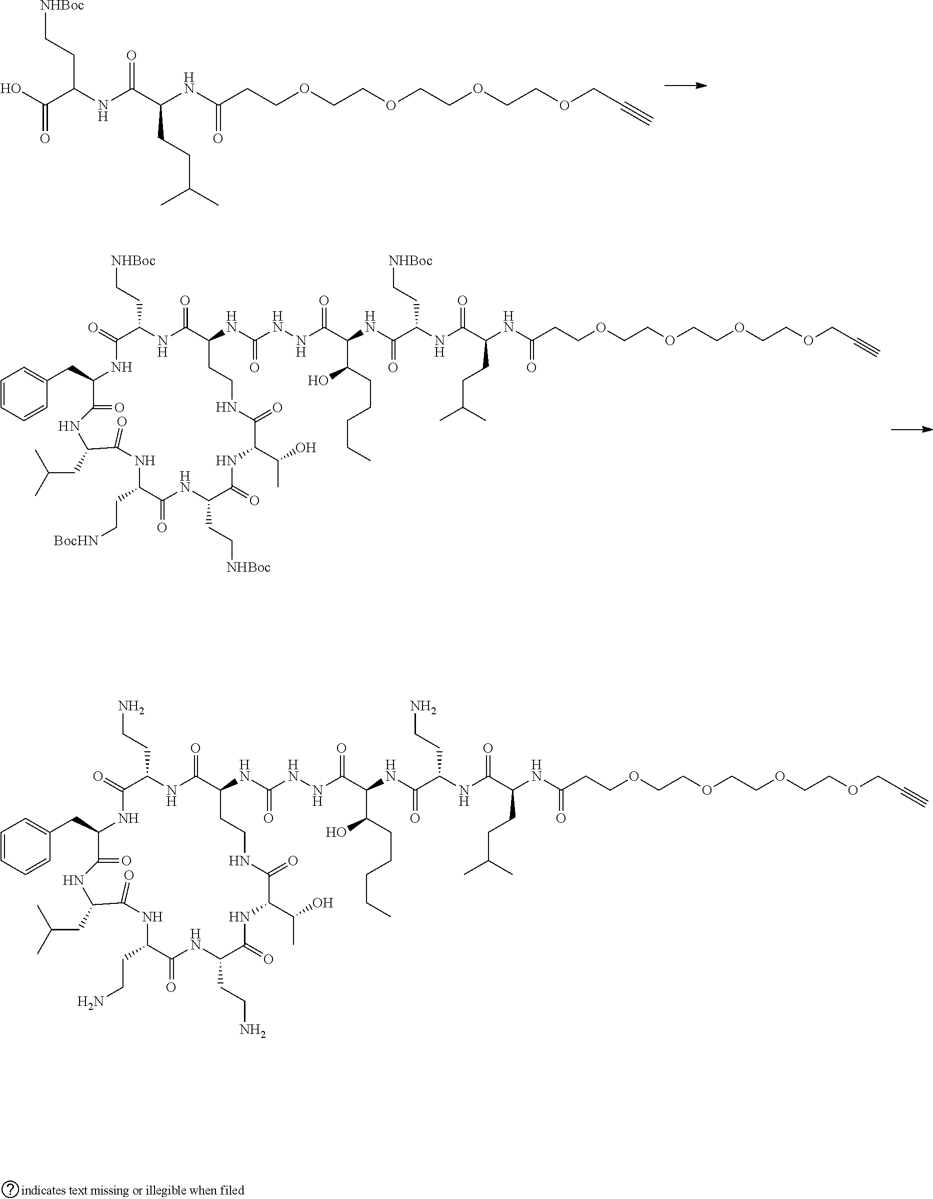

### Step a

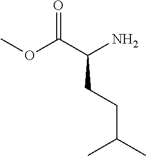

Solid (2S)-2-amino-5-methylhexanoic acid (1.5 g, 10.33 mmol) was added to a round bottom flask under nitrogen gas and at 0° C. To this flask was added anhydrous methanol following by slow addition of trimethylsilylchloride (5.24 mL, 41.32 mmol) (11 mL). The reaction mixture was stirred at 0° C. for 10 minutes then the ice bath was removed and stirred at room temperature overnight. After the reaction was complete, volatiles were removed under reduced pressure to give a yellow foam. This material was used in the next step without further purification. Yield of product was 1.99 g, 121%. Ion(s) found by LCMS: (M+H)+=160.2

### Step b

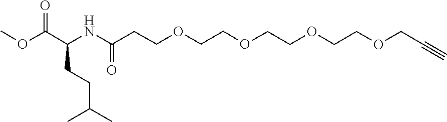 [SMILES: C#CCOCCOCCOCCOCCC(=O)N[C@@H](CCC(C)C)C(=O)OC]

A solution of the amine HCl salt from the previous step (0.95 g, 4.61 mmol), acid-PEG4-alkyne (1.0 g, 3.84 mmol), EDCI (2.21 g, 11.53 mmol), HOAt (1.58 g, 11.53 mmol), and DIEA (4.09 mL, 23.05 mmol) in DMF (9.5 mL) was stirred at room temperature overnight. After the reaction was completed, it was purified by reversed phase liquid chromatography (RPLC) using Isco CombiFlash liquid chromatography eluted with 5% to 40% acetonitrile and water, using 0.1% TFA as modifier. Yield of product was 0.83 g, 54%. Ion(s) found by LCMS: (M+H)+=401.9

### Step c

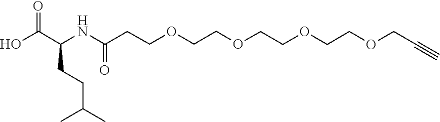 [SMILES: C#CCOCCOCCOCCOCCC(=O)N[C@@H](CCC(C)C)C(=O)O]

A solution of the methyl ester from the previous step (0.83 g, 2.07 mmol) and LiOH (0.074 g, 3.11 mmol) in MeOH:THF:H2O (1:1:2.12 mL) was stirred at room temperature until complete by LCMS. The reaction was acidified with Amberlite®IRN-77, filtered, and washed with methanol then concentrated under reduced pressure. This material was used in the next step without purification. Yield of product was 0.82 g, 101%. Ion(s) found by LCMS: (M−H)+=386.2

### Step d

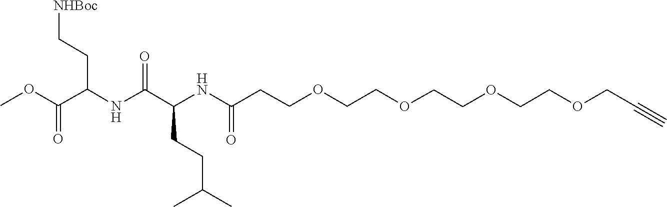 [SMILES: C#CCOCCOCCOCCOCCC(=O)N[C@@H](CCC(C)C)C(=O)NC(CCNC(=O)OC(C)(C)C)C(=O)OC]

A solution of the acid from the previous step (0.50 g, 1.29 mmol), methyl (2S)-2-amino-4-[(tert-butoxy)carbonylamino]butanoate HCl salt (0.42 g, 1.55 mmol), HOAt (0.53 g, 3.87 mmol), EDCI (0.74 g, 3.87 mmol) and DIEA (1.4 mL, 7.74 mmol) in DMF (3 mL) was stirred at room temperature overnight. The reaction mixture was purified by reversed phase liquid chromatography (RPLC) using Isco CombiFlash liquid chromatography eluted with 5% to 60% acetonitrile and water, using 0.1% TFA as modifier. Yield of product was 0.63 g, 81%. Ion(s) found by LCMS: (M+H)+=602.3

### Step e

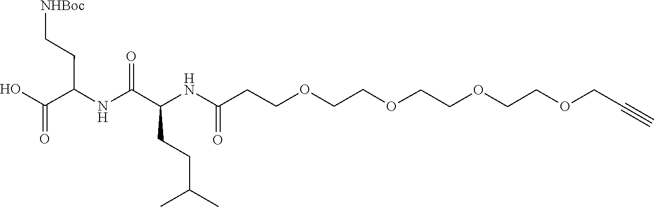 [SMILES: C#CCOCCOCCOCCOCCC(=O)N[C@@H](CCC(C)C)C(=O)NC(CCNC(=O)OC(C)(C)C)C(=O)O]

A solution of the methyl ester from the previous step (0.63 g, 1.04 mmol) and LiOH (0.037 g, 1.57 mmol) in MeOH:THF:H2O (1:1:2, 12 mL) was stirred at room temperature until complete by LCMS. After the reaction was complete, it was acidified with Amberlite® IRN-77 then filtered and washed with methanol. The filtrate was concentrated and used in the next step without further purification. Yield of product was 0.60 g, 98%. Ion(s) found by LCMS: (M−H)+=586.2

### Step f

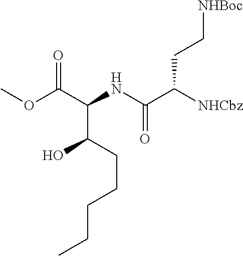 [SMILES: CCCCC[C@@H](O)[C@H](NC(=O)[C@H](CCNC(=O)OC(C)(C)C)NC(=O)OCc1ccccc1)C(=O)OC]

Synthesis of methyl (2S,3R)-2-amino-3-hydroxyoctanoate: Synthesis of the extended Threonine is described in the following reference: David Aiker, Giles Hamblett, Laurence M. Harwood, Sarah M. Robertson, David J. Watkin and Eleri Williams, Tetrahedron 54, 6089-98 (1998).

A solution of methyl (2S,3R)-2-amino-3-hydroxyoctanoate (2.50 g, 13.22 mmol), (2S)-4-[(tert-butoxy)carbonylamino]-2-[(phenylmethoxy)carbonylamino]butanoic acid (4.89 g, 13.88 mmol), EDCI (3.80 g, 19.82 mmol), HOBt (2.73 g, 19.82 mmol) in CH₂Cl₂:DMF (4:1, 15 mL) was stirred at 0° C. under nitrogen gas then NaHCO₃ (2.27 g, 26.43 mmol) was added. The ice bath was removed, and the reaction was stirred at room temperature for 3 hours and monitored by analytical HPLC. After the reaction was completed by LCMS, it was diluted with ethyl acetate and washed with 1N HCl aqueous, saturated NaHCO₃ aqueous and brine. The organic layer was dried over Na₂SO₄ and filtered then concentrated. The residue was purified by normal phase liquid chromatography (NPLC) using Isco CombiFlash liquid chromatography eluted with 0% to 3% dichloromethane and methanol. Yield of product was 5.79 g, 84%. Ion(s) found by LCMS: (M+H)+=424.3

### Step g

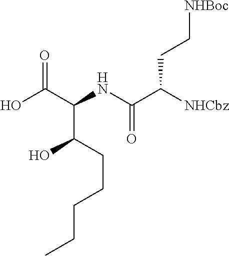 [SMILES: CCCCC[C@@H](O)[C@H](NC(=O)[C@H](CCNC(=O)OC(C)(C)C)NC(=O)OCc1ccccc1)C(=O)O]

A solution of the methyl ester from the previous step (5.78 g, 11.04 mmol) and LiOH (0.28 g, 11.59 mmol) in MeOH:THF:H2O (1:1:2, 28 mL) was stirred at room temperature until completion. The reaction was monitored by LCMS. After the reaction was complete, it was acidified with 1N HCl then volatiles were removed under reduced pressure. The residue in water was dissolved with acetonitrile then lyophilized. Yield of product was 5.65 g, 100%. Ion(s) found by LCMS: (M−Boc+H)+=410.2

### Step h

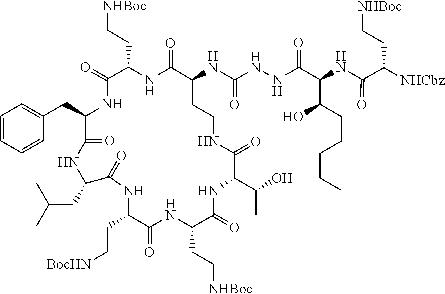 [SMILES: CCCCC[C@@H](O)[C@H](NC(=O)[C@H](CCNC(=O)OC(C)(C)C)NC(=O)OCc1ccccc1)C(=O)NNC(=O)N[C@H]1CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](Cc2ccccc2)NC(=O)[C@H](CCNC(=O)OC(C)(C)C)NC1=O]

A solution of acid from a previous step (0.77 g, 1.47 mmol), tri-Boc PMBH-azagly-NH2 (Step-b product in Example 192; 1.5 g, 1.34 mmol), HOAt (0.37 g, 2.68 mmol), DCC (0.55 g, 2.68 mmol) and DIEA (1.4 mL, 8.03 mmol) in DMF:CH₂Cl₂ (6:1, 14 mL) was stirred at room temperature overnight. Volatiles were removed under reduced pressure. The residue was purified by reversed phase liquid chromatography (RPLC) using Isco CombiFlash liquid chromatography eluted with 10% to 70% acetonitrile and water, using 0.1% TFA as modifier. Yield of product was 1.70 g, 79%. Ion(s) found by LCMS: [(M−2Boc+H)/2]+=706.6

### Step i

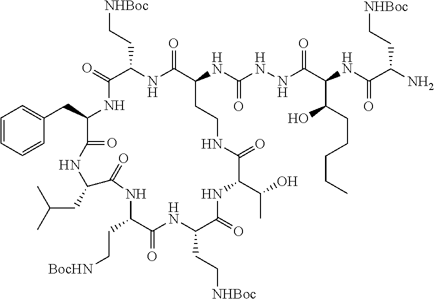 [SMILES: CCCCC[C@@H](O)[C@H](NC(=O)[C@@H](N)CCNC(=O)OC(C)(C)C)C(=O)NNC(=O)N[C@H]1CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](Cc2ccccc2)NC(=O)[C@H](CCNC(=O)OC(C)(C)C)NC1=O]

A solution of the Cbz-protected amine from the previous step (1.7 g, 1.05 mmol) in MeOH (17 mL) and CHCl₃ (0.086 mL) at room temperature was charged with Silica(cat)Pd(0) (0.79 g, 0.16 mmol) and hydrogen gas balloon. The reaction was stirred at room temperature overnight. The reaction mixture was filtered through a pad of Celite®, washed with methanol then concentrated. The residue was purified by reversed phase liquid chromatography (RPLC) using Isco CombiFlash liquid chromatography eluted with 5% to 70% acetonitrile and water. Yield of product was 0.24 g, 16%. Ion(s) found by LCMS: [(M−2Boc+H)/2]+=639.6

### Step j

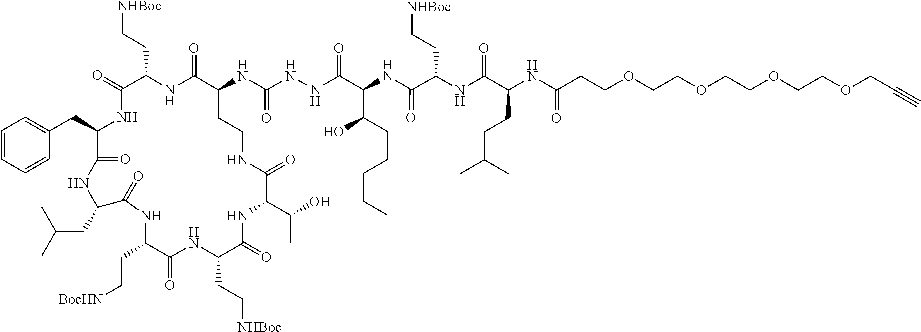 [SMILES: C#CCOCCOCCOCCOCCC(=O)N[C@@H](CCC(C)C)C(=O)N[C@@H](CCNC(=O)OC(C)(C)C)C(=O)N[C@H](C(=O)NNC(=O)N[C@H]1CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](Cc2ccccc2)NC(=O)[C@H](CCNC(=O)OC(C)(C)C)NC1=O)[C@H](O)CCCCC]

A solution of the amine from the previous step (0.24 g, 0.16 mmol), acid from step e (0.07 g, 0.18 mmol), DCC (0.10 g, 0.49 mmol), HOAt (0.037 g, 0.16 mmol) and DIEA (0.17 mL, 0.99 mmol) in DMF:CH₂Cl₂ (5 mL:1 mL) was stirred at room temperature overnight. After the reaction was complete, volatiles were removed under reduced pressure. The resulting residue was purified by reversed phase liquid chromatography (RPLC), using Isco CombiFlash liquid chromatography eluted with 10% to 100% methanol and water, using 0.1% TFA as modifier. Yield of product was 0.28 g, 91%. Ion(s) found by LCMS: [(M−2Boc+H)/2]+=824.4

### Step k

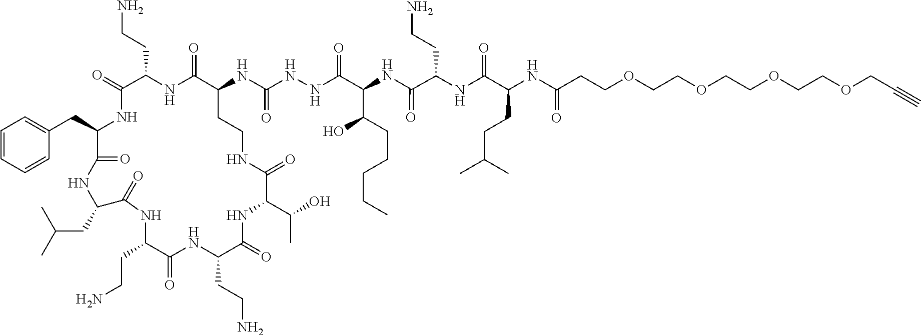 [SMILES: C#CCOCCOCCOCCOCCC(=O)N[C@@H](CCC(C)C)C(=O)N[C@@H](CCN)C(=O)N[C@H](C(=O)NNC(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](O)CCCCC]

A solution of the Boc-protected product from the previous step (0.71 g, 0.38 mmol) and TFA (6 mL) in CH₂Cl₂ (4 mL) was stirred at room temperature for 1 hour then concentrated under reduced pressure. The residue was purified by reversed phase high pressure liquid chromatography (HPLC) using ACCQ-Prep liquid chromatography eluted with 5% to 35% acetonitrile and water, using 0.1% TFA as modifier. Yield of product was 0.39 g, 71%. Ion(s) found by LCMS: [(M+H)/2]+=724.0, [(M+H)/3]+=483.0, [(M+H)/4]+=362.6.

### Example 227—Synthesis of Int-117

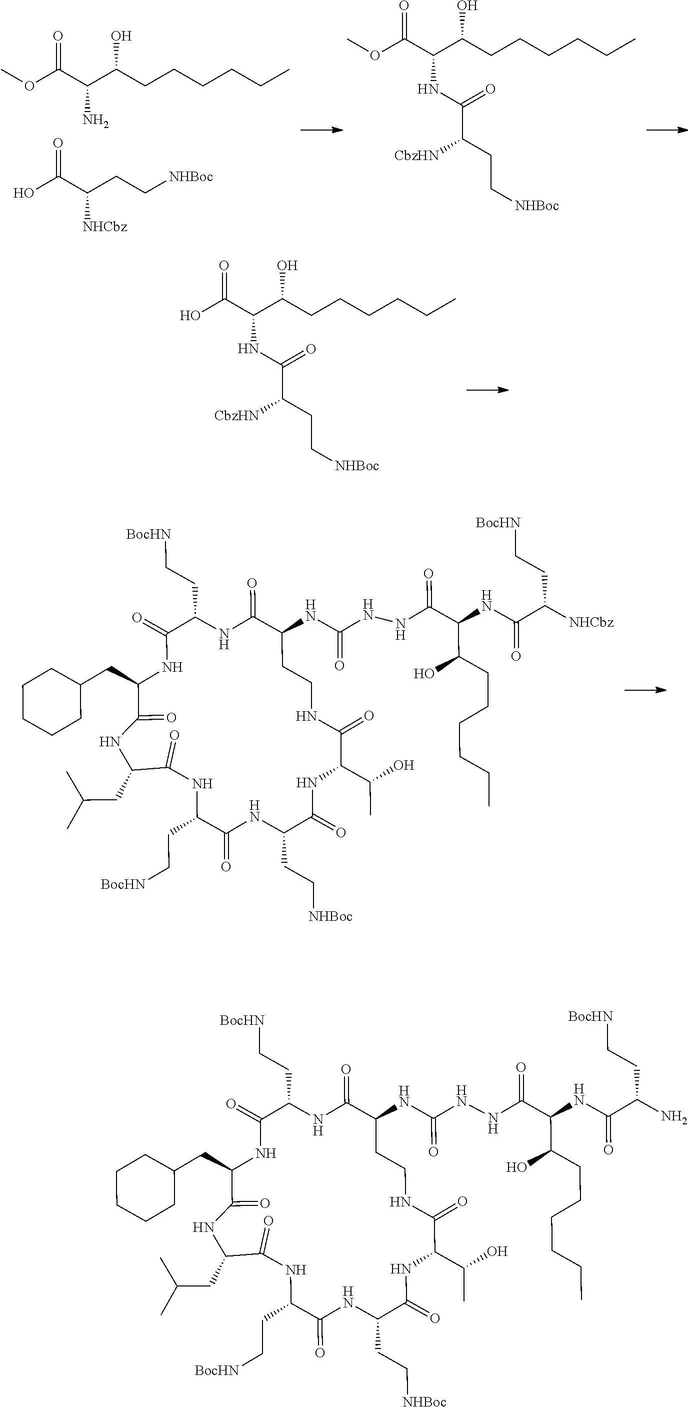

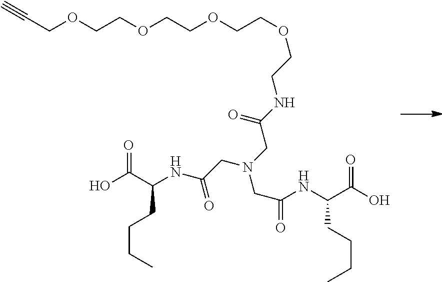 [SMILES: C#CCOCCOCCOCCOCCNC(=O)CN(CC(=O)N[C@@H](CCCC)C(=O)O)CC(=O)N[C@@H](CCCC)C(=O)O]

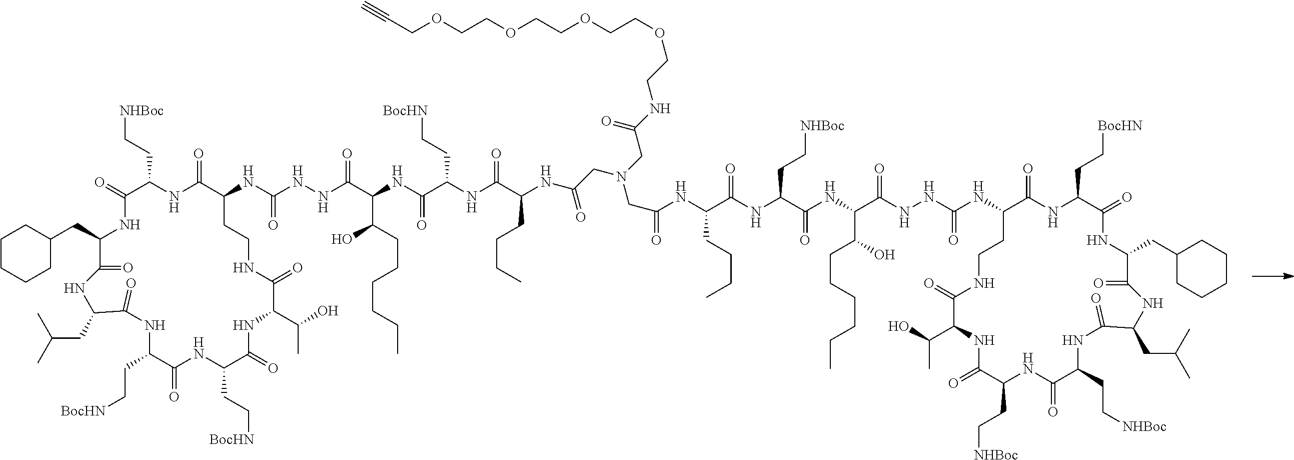

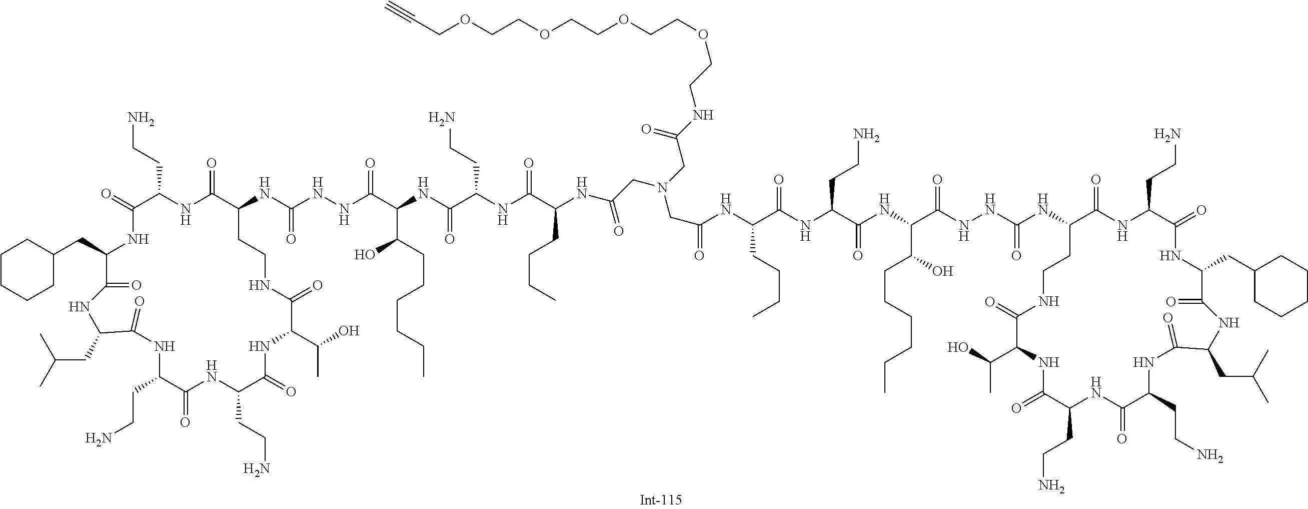 [SMILES: C#CCOCCOCCOCCOCCNC(=O)CN(CC(=O)N[C@@H](CCCC)C(=O)N[C@@H](CCN)C(=O)N[C@H](C(=O)NNC(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](CC2CCCCC2)NC(=O)[C@H](CCN)NC1=O)[C@H](O)CCCCCC)CC(=O)N[C@@H](CCCC)C(=O)N[C@@H](CCN)C(=O)N[C@H](C(=O)NNC(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](CC2CCCCC2)NC(=O)[C@H](CCN)NC1=O)[C@H](O)CCCCCC]

### Step a

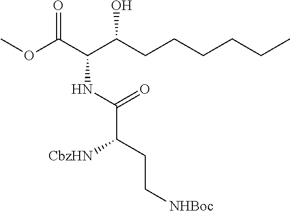 [SMILES: CCCCCC[C@@H](O)[C@H](NC(=O)[C@H](CCNC(=O)OC(C)(C)C)NC(=O)OCc1ccccc1)C(=O)OC]

To a mixture of Nα-Z-Nγ-Boc-L-2,4-diaminobutyric acid (1.41 g, 4 mmol) and C5-extended Thr-OMe (1.068 g, 4.4 mmol) in anhydrous DMF (4 mL) was added HATU (1.52 g, 4 mmol) in portions over 10 minutes, followed by DIPEA (520 mg, 4 mmol). The resulting mixture was stirred for 30 minutes, then purified through RPLC (50 g, 30 to 60% acetonitrile and water). Yield 2.01 g, 93.5%. Ion found by LCMS: [M−Boc+H]⁺=438.2.

### Step b

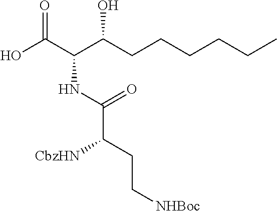 [SMILES: CCCCCC[C@@H](O)[C@H](NC(=O)[C@H](CCNC(=O)OC(C)(C)C)NC(=O)OCc1ccccc1)C(=O)O]

A solution of the step-a product (2.01 g, 3.74 mmol) in MeOH:THF (1:1, 10 mL) was cooled in an ice-water bath and added in portions a solution of LiOH monohydrate (168 mg, 4 mmol) in water (4 mL) over 1 hour. After the reaction mixture was stirred for 2 more hours, it was slightly acidified by 4N HCl solution in dioxane (1 mL) and purified by RPLC (100 g SiO₂, 0% to 65% acetonitrile and water). Yield 1.91 g, 97.7%. Ions found by LCMS: [M-Boc+H]⁺=424.2.

### Step c

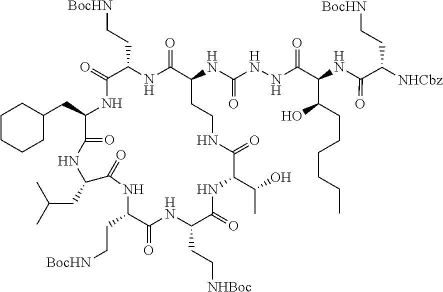 [SMILES: CCCCCC[C@@H](O)[C@H](NC(=O)[C@H](CCNC(=O)OC(C)(C)C)NC(=O)OCc1ccccc1)C(=O)NNC(=O)N[C@H]1CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](CC2CCCCC2)NC(=O)[C@H](CCNC(=O)OC(C)(C)C)NC1=O]

To a mixture of the step-b product (188.5 mg, 0.36 mmol) and the step-c product (337.9 mg, 0.3 mmol, described in Example 225 Int-115) in anhydrous DMF (1 mL) was added HATU (136.9 mg, 0.36 mmol) in portions over 5 minutes, followed by DIPEA (65 mg, 0.5 mmol). The resulting mixture was stirred for 1 hour, then purified by RPLC (50 g SiO₂, 30% to 90% MeOH and water). Yield 276.6 mg, 56.7%. Ion found by LCMS: [(M−2Boc+2H)/2]⁺=716.4.

### Step d

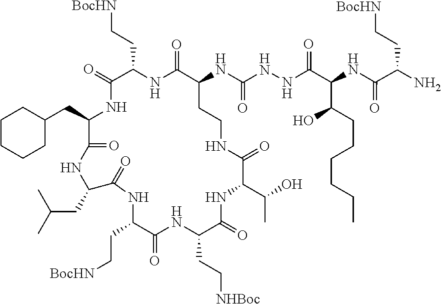 [SMILES: CCCCCC[C@@H](O)[C@H](NC(=O)[C@@H](N)CCNC(=O)OC(C)(C)C)C(=O)NNC(=O)N[C@H]1CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](CC2CCCCC2)NC(=O)[C@H](CCNC(=O)OC(C)(C)C)NC1=O]

A 50 mL reaction flask was flushed with nitrogen and charged with the step-c product (276.6 mg, 0.17 mmol), MeOH (20 mL) and 4N HCl in dioxane (0.05 mL). Pd/C (95 mg) was added, and the resulting mixture was stirred under a hydrogen atmosphere for 2 hours. Pd/C was filtered, and the filtrate was concentrated by rotary evaporation. The residue was then purified by RPLC (100 g SiO₂, 30% to 85% MeOH and water). Yield 178.7 mg, 70.2%. Ion found by LCMS: [(M−Boc+2H)/2]+=699.4.

### Step e

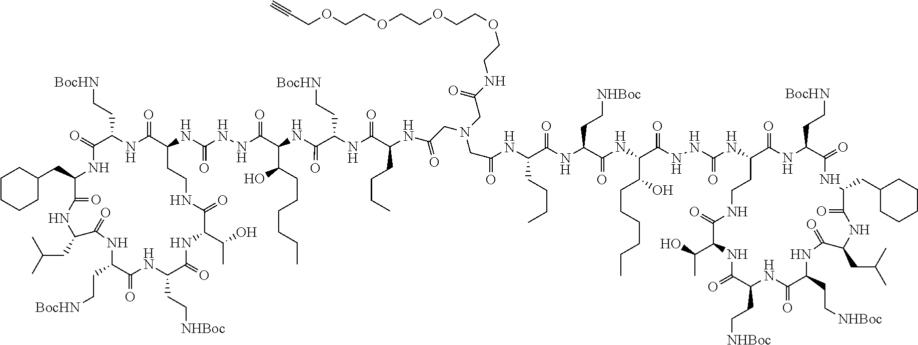

To a solution of the step-d product from Example 77 (Synthesis of Int-42) (28.4 mg, 0.045 mmol) and the step-d product from above (178.7 mg, 0.119 mmol) in anhydrous DMF (1 mL) was added HATU (34.2 mg, 0.09 mmol). After stirred for 5 minutes, DIPEA (52 mg, 0.4 mmol) was added and the reaction mixture was stirred for 30 minutes. It was then directly purified by RPLC (50 g SiO₂, 40% to 100% MeOH and water, using 0.1% TFA as modifier). Yield 157.3 mg, 97.8%. Ion found by LCMS: [(M−3Boc+3H)/3]⁺=1097.

### Step f

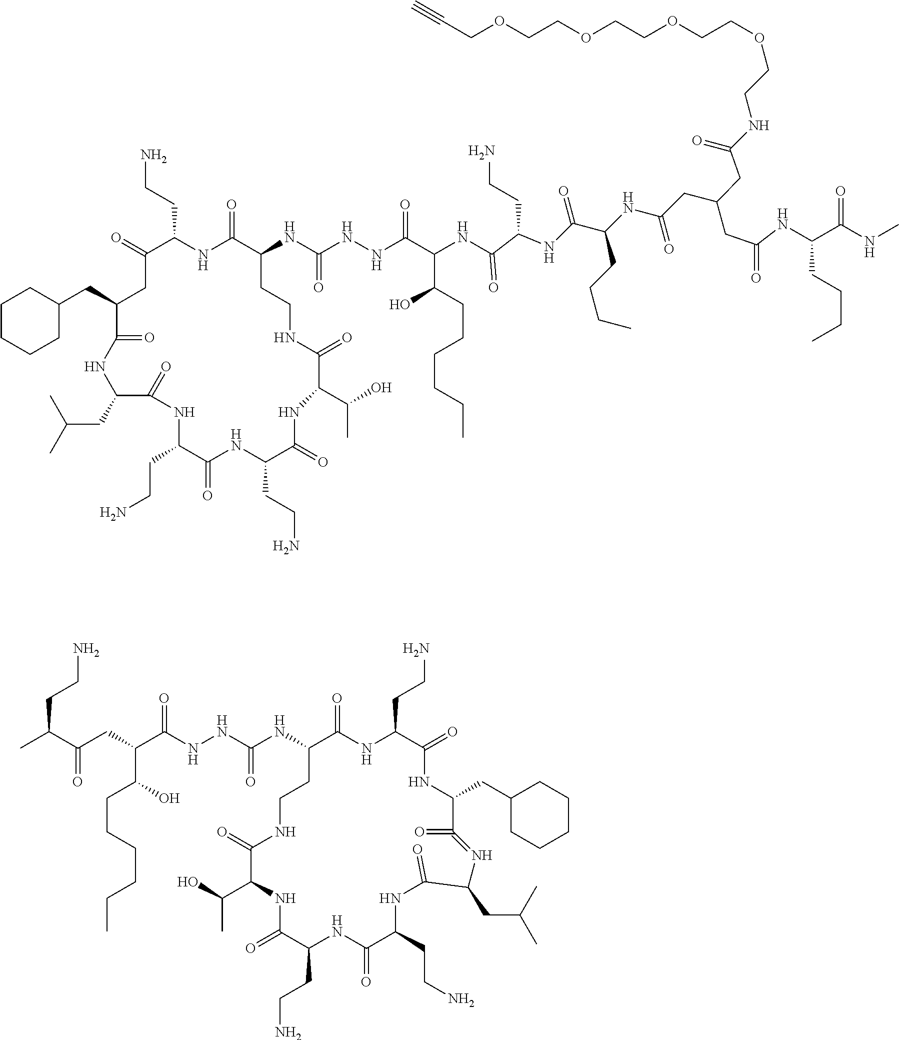

The step-e product from above (157.3 mg, 0.044 mmol) was dissolved in DCM:TFA (1:1, 1 mL). The solution was stirred at 30° C. for 20 minutes. It was then concentrated and purified by HPLC (0% to 25% acetonitrile and water, using 0.1% TFA as modifier). The yield of Int-117 was 105.9 mg, 65%. Ions found by LCMS: [(M+3H)/3]⁺=930.5, [(M+4H)/4]⁺=698.1, [(M+5H)/5]⁺=558.8, [(M+6H)/6]⁺=465.8, [(M+7H)/7]⁺=399.4.

### Example 228—Synthesis of Int-118

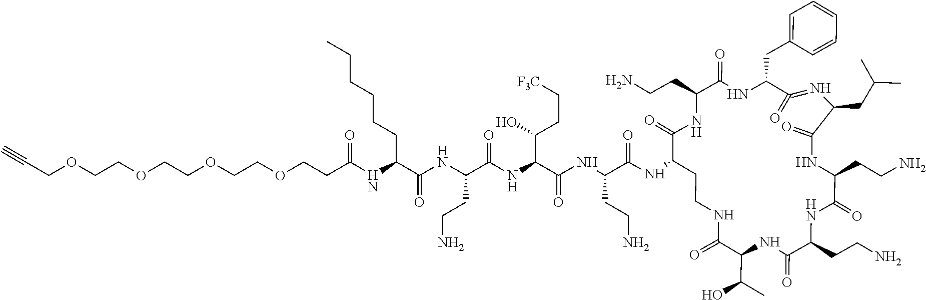

The title compound was prepared analogously to Example 193 (Int-93), where the hexanal was substituted with 4,4,4-trifluorobutanal in step a of the preparation of the methyl ester of the extended threonine. Ion(s) found by LCMS: [(M+2H)/2]+=764.9, [(M+3H)/3]+=510.3, [(M+4H)/4]+1=382.9.

### Example 229—Synthesis of Int-119

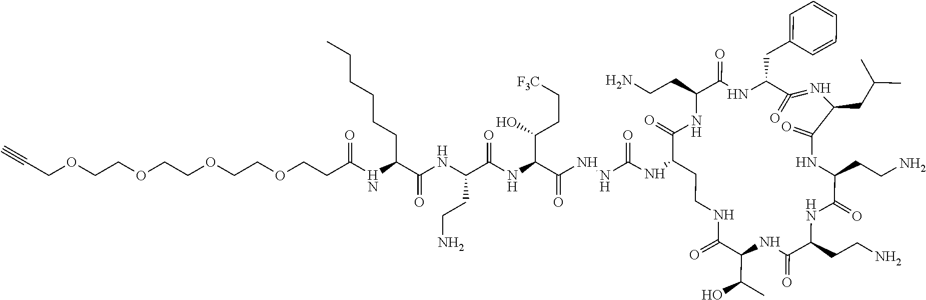

The title compound was prepared analogously to Example 228 Int-118, where the tetra-Boc L-Dab-polymyxin B cycloheptapeptide (product of Step-f, Example 67) was substituted with the Tri-Boc aza-gly-polymyxin B cycloheptapeptide (Step-b product, Example 192). Ion(s) found by LCMS: [(M+2H)/2]+=743.9, [(M+3H)/3]+=496.3, [(M+4H)/4]+=372.5.

### Example 230—Synthesis of Int-120

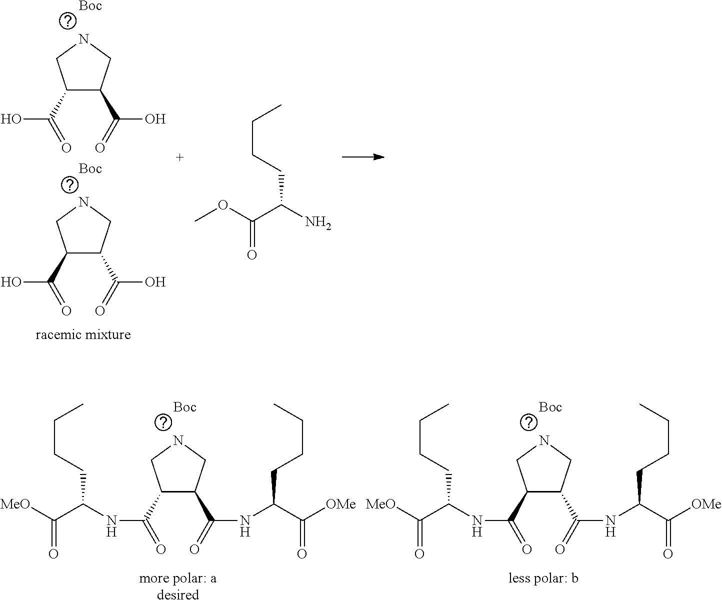

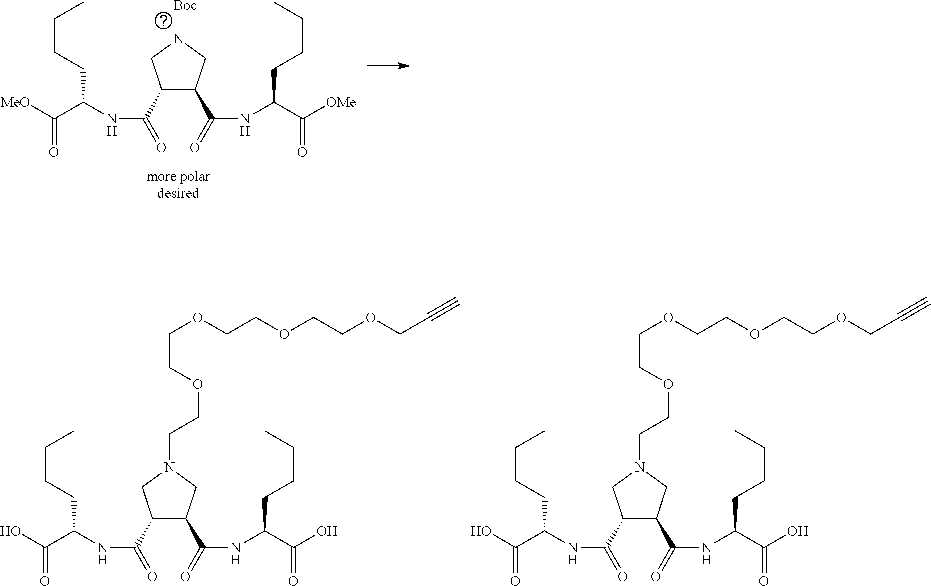 [SMILES: CCCC[C@H](NC(=O)[C@@H]1CNC[C@H]1C(=O)N[C@@H](CCCC)C(=O)OC)C(=O)OC ; "Boc" ; C#CCOCCOCCOCCOCCN1C[C@@H](C(=O)N[C@@H](CCCC)C(=O)O)[C@H](C(=O)N[C@@H](CCCC)C(=O)O)C1 ; C#CCOCCOCCOCCOCCN1C[C@@H](C(=O)N[C@@H](CCCC)C(=O)O)[C@H](C(=O)N[C@@H](CCCC)C(=O)O)C1]

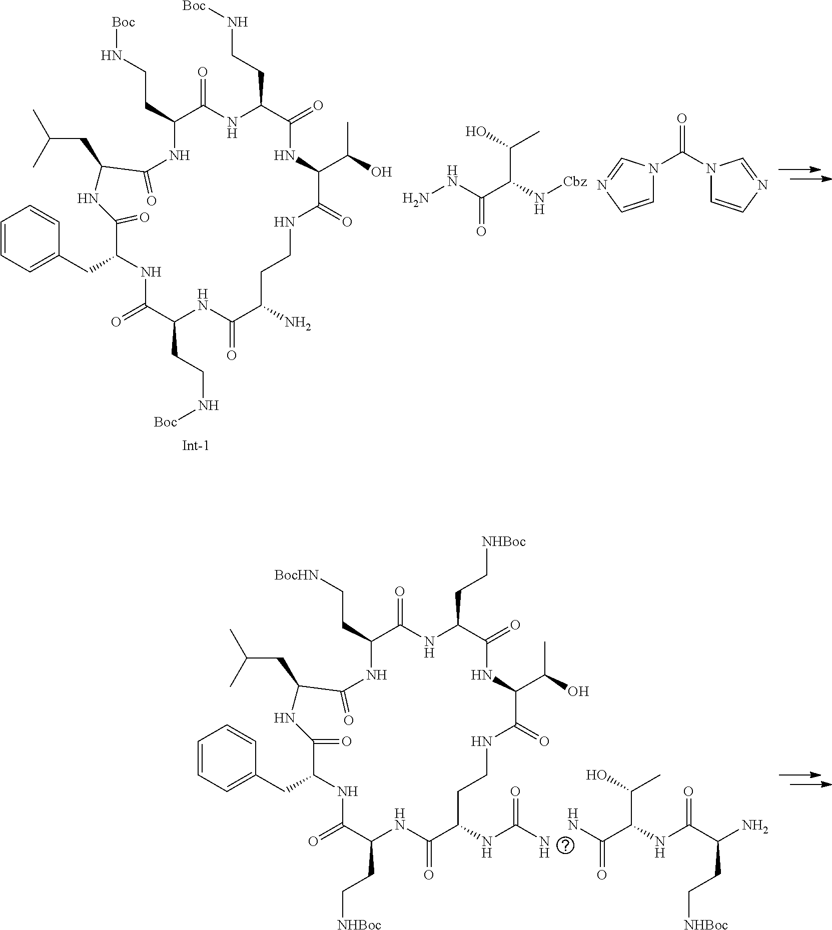

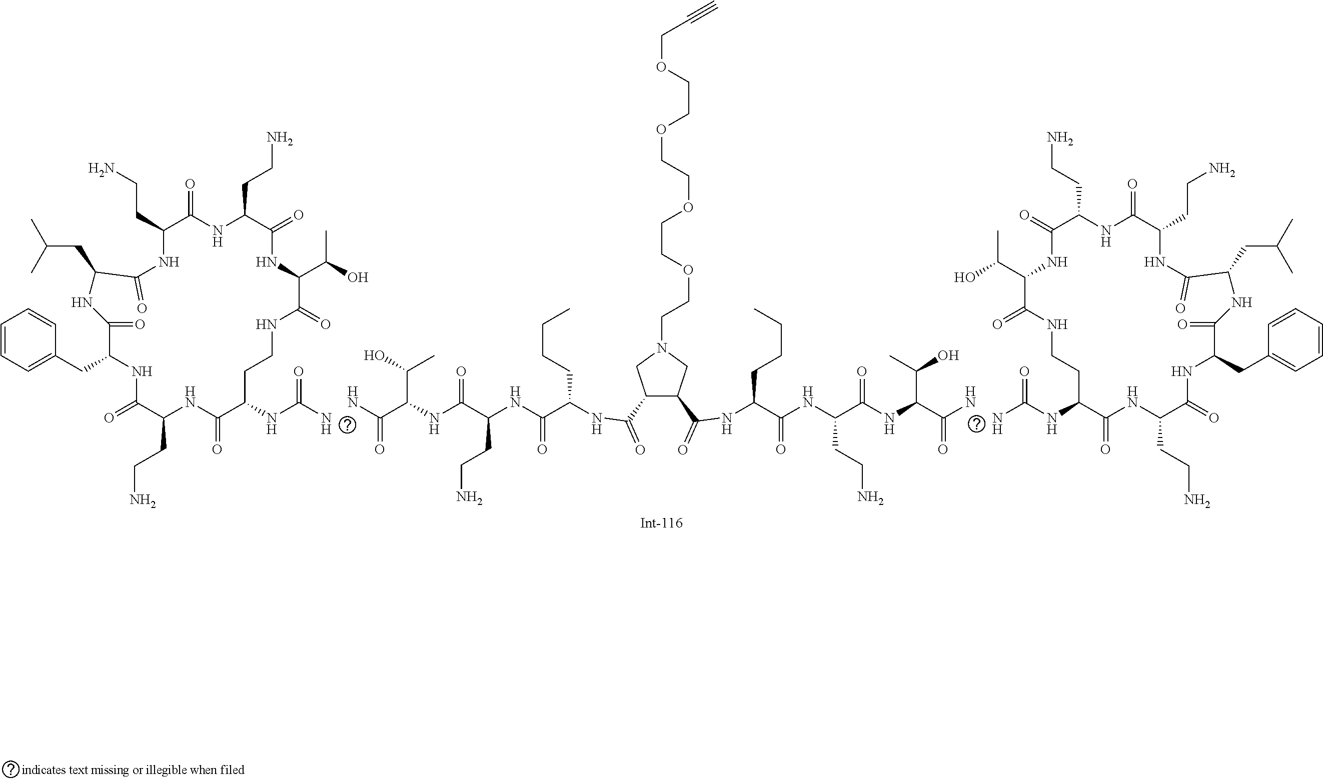

### Step a. Synthesis of Pyrolidine Diamide

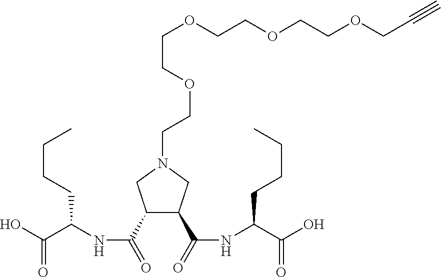 [SMILES: C#CCOCCOCCOCCOCCN1C[C@@H](C(=O)N[C@@H](CCCC)C(=O)O)[C@H](C(=O)N[C@@H](CCCC)C(=O)O)C1]

Racemic-trans1-(tert-butoxycarbonyl)pyrrolidine-3,4-dicarboxylic acid (15.00 g, 57.86 mmol), H-norleu-OMe-hydrochloride (21.02 g, 115.7 mmol), and sodium bicarbonate (19.44 g, 231.4 mmol) were dissolved in DCM/DMF (5:1, 100 mL) followed by addition of EDCI (27.73 g, 144.6 mmol) and HOAt (19.69 g, 144.6 mmol). The mixture was stirred for 5 hours then most of the DCM was removed and the residue was precipitated out in 0.5 N aq. HCl (300 mL). The dried solid was subjected to reversed phase liquid chromatography (RPLC) using an Isco Combiflash liquid chromatograph eluted with 40% to 60% acetonitrile and water using 0.1% TFA modifier. The two diastereomers were separated, pooled and lyophilized separately into the more polar diastereomer (a) and the less polar diastereomer (b):Ion found by LC/MS [M+H]⁺=414.2 (loss of 1 Boc group) for both Boc-protected intermediates. Yield: 77%. The desired diastereomer (a) was stirred in 4N HCl in dioxane (40 mL) for 60 minutes then concentrated to give free amine HCl salt. Ion found by LC/MS [M+H]⁺=414.2. (The stereochemistry of the polar diastereomer was assigned using single crystal x-ray crystallography. Results not shown.) The HCl salt of the amine was dissolved in dry DMF (20 mL) followed by addition of DIPEA (1.64 mL, 9.39 mmol). The solution was heated at 50° C. Propargyl-PEG4-bromide (1.220 g, 4.133 mmol) in 8 mL of DMF was added via syringe pump at 2 mL/hr rate. After the addition of propargyl-PEG4-bromide the reaction mixture was heated at 50° C. for overnight and was cooled to room temperature. DMF was removed and the residue was purified by reversed phase liquid chromatography (RPLC) using an Isco Combiflash liquid chromatograph eluted with 25% to 75% acetonitrile and water without TFA modifier to give 1.54 g of a light color oil, 65% yield. The resulting dimethyl ester was treated with LiOH (0.127 g, 5.152 mmol) in THF/H₂O (1:1) for 1 hour. It was acidified by using Dowex acidic resin. The mixture was filtered and washed with methanol. After concentrating, the di-acid was pure and used for next step without further purification. Yield 100%. Ion found by LCMS: [M+H]+=600.

### Step b. Synthesis of tetra-Boc-azagly-PMBD-NH2

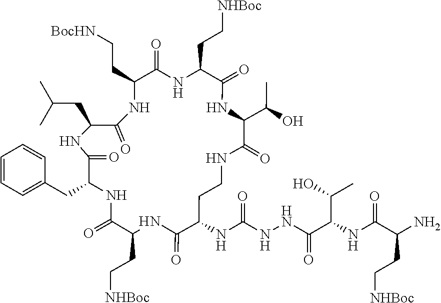 [SMILES: CC(C)C[C@@H]1NC(=O)[C@@H](Cc2ccccc2)NC(=O)[C@H](CCNC(=O)OC(C)(C)C)NC(=O)[C@@H](NC(=O)NNC(=O)[C@@H](NC(=O)[C@@H](N)CCNC(=O)OC(C)(C)C)[C@@H](C)O)CCNC(=O)[C@H]([C@@H](C)O)NC(=O)[C@H](CCNC(=O)OC(C)(C)C)NC(=O)[C@H](CCNC(=O)OC(C)(C)C)NC1=O]

Cbz-Thr-NH—NH2 (7.548 g, 28.24 mmol), CDI (4.579 g, 28.24 mmol), NMM (3.10 mL, 28.24 mmol) and catalytic DMAP (0.230 g, 1.88 mmol) were dissolved in DMF (18 mL) at 80° C. Int-1 (10.00 g, 9.413 mmol) was added and the mixture was stirred for overnight at 80° C. Precipitate was removed by filtration and the filtrate was concentrated and purified by reversed phase liquid chromatography (RPLC) using an Isco Combiflash liquid chromatograph eluted with 30% to 75% acetonitrile and water with 0.1% TFA modifier to give tri-Boc-aza-gly-PMBN-NH-Cbz as a white solid, 11.15 g, 87% yield. Ions found by LCMS: [M−t−Bu−2Boc+2H]⁺/2=550.3, [M−2Boc+2H⁺/2=578.4. The Cbz group of this fully protected aza-gly-PMBN was removed by hydrogenolysis with Pd/C in 0.5% of (CHCl₃/methanol). The product was pure enough for next step reaction without purification. Ions found by LCMS: [M−t−Bu−2Boc+2H⁺]/2=483.4, [M−2Boc+2H⁺/2=511.4, [M-t-Bu-Boc+2H⁺]/2=533.4, [M−2Boc+2H⁺]/2=511.4, [M+2H⁺]/2=561.4, [M+H⁺]=1222.4. A flask was charged with tri-Boc-PMBN-(aza-gly)-Thr-NH2 (11.675 g, 9.559 mmol), Z-Dab(Boc)-OH (3.537, 10.34 mmol), EDCI (2.749 g, 14.34 mmol), HOAt (1.952 g, 14.34 mmol) dissolved in DMF (20 mL) followed by addition of DIPEA (5.10 mL, 28.68 mmol). The reaction mixture was stirred overnight at room temperature. Most DMF was removed under reduced pressure. The residue was dumped into 0.5N aq. HCl to form a precipitate. After filtration and drying, a white solid was obtained. LCMS shows the following ions: [M−4Boc+2H⁺]/2=578.3, [M−t−Bu−2Boc+2H⁺]/2=650.5, [M−2Boc+2H⁺]/2=678.4. The Cbz group of this aza-gly-PMBD was removed by hydrogenolysis with Pd/C in ethanol/ with 2 eq HCl. After removal of ethanol, the residue was purified by reversed phase liquid chromatography (RPLC) using an Isco Combiflash liquid chromatograph eluted with 30% to 75% acetonitrile and water without TFA modifier to give tetra-Boc-azagly-PMBD-NH₂ as a white solid, 10.56 g 78% yield. Ions found by LCMS: [M−3Boc+2H⁺]/2=561.4, [M−2Boc+2H+/2=661.4, [M+H⁺]=1222.4.

### Step c. Synthesis of Int-120

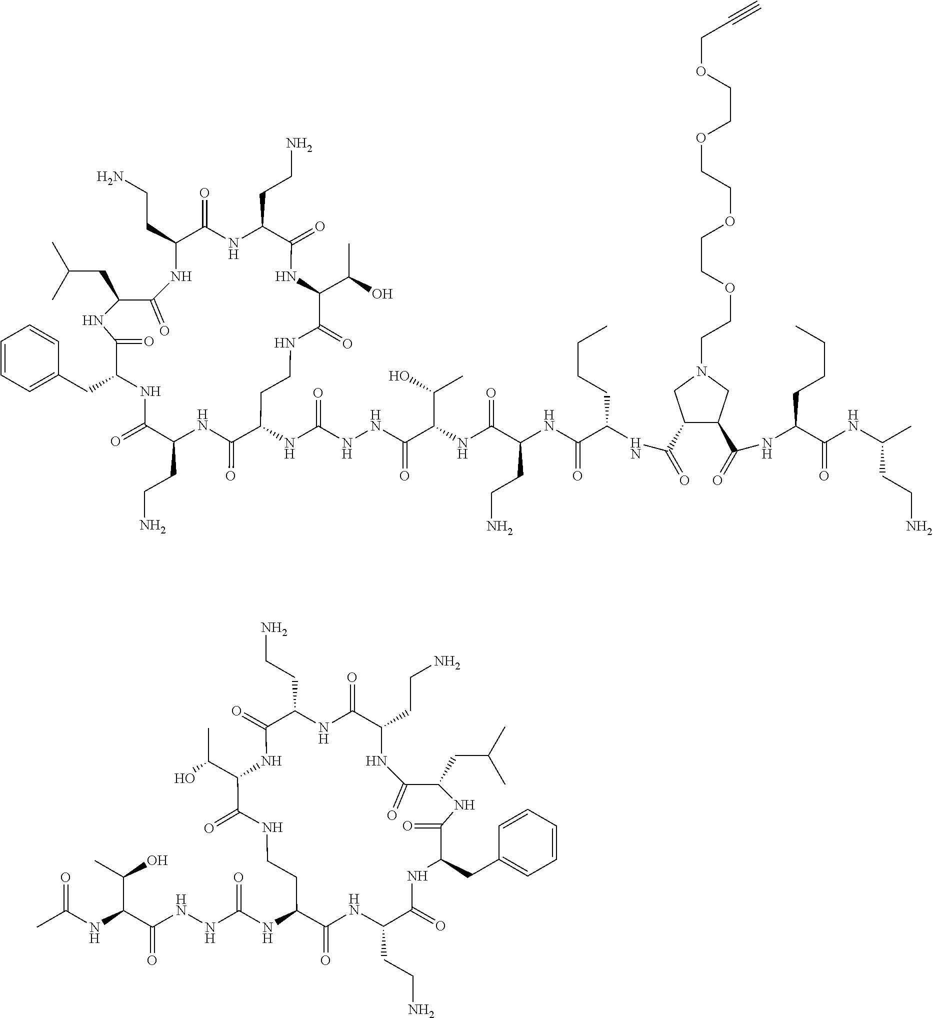 [SMILES: C#CCOCCOCCOCCOCCN1C[C@@H](C(=O)N[C@@H](CCCC)C(=O)N[C@H](C)CCN)[C@H](C(=O)N[C@@H](CCCC)C(=O)N[C@@H](CCN)C(=O)N[C@H](C(=O)NNC(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)C1 ; CC(=O)N[C@H](C(=O)NNC(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O]

PMBD-(aza-gly)-thr-NH2 (2.250 g, 1.583 mmol), diacid central linker from step a (0.475 g, 0.791 mmol), EDCI (0.379 g, 1.98 mmol), HOAt (0.289 g, 1.98 mmol) and DIPEA (0.58 mL, 3.2 mmol) were mixed. The reaction solution was stirred overnight. The reaction was concentrated then dissolved in minimum amount of methanol and purified by reversed phase liquid chromatography (RPLC) using an Isco CombiFlash liquid chromatograph eluted with 60% to 100% acetonitrile and water using 0.1% TFA modifier to give 1.47 g white solid. Ions found by LCMS: (M−3 Boc+3H⁺)/3=1038.2 (M−2 Boc+3H⁺)/3=1069.5. The white solid was treated with 30 mL of TFA for 30 min followed by removal of excess TA. The residue was applied to reversed phase liquid chromatography (RPLC) using an Isco CombiFlash liquid chromatograph eluted with 5% to 45% acetonitrile and water using 0.1% TFA modifier to give the desired product (Int-120) as a white powder. Yield 90%. Ion(s) found by LCMS: (M+3H⁺)/3=869.4. (M+4H⁺)/4=652.4, (M+5H⁺)/5=522.4, (M+6H⁺)/6=435.4.

### Example 231—Synthesis of Int-121

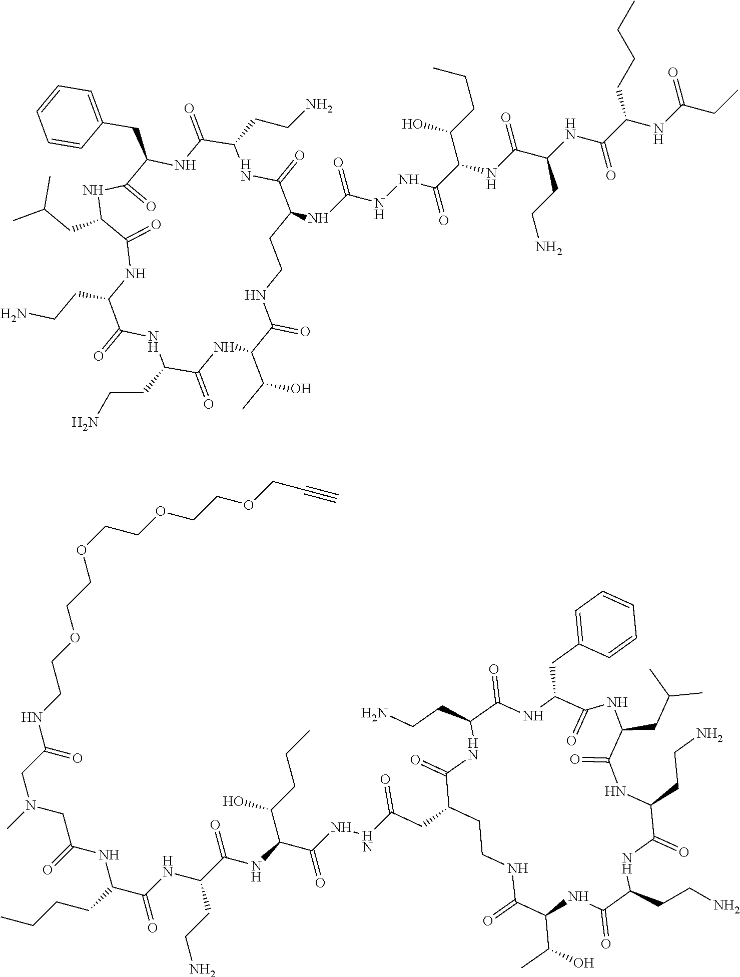

The title compound (Int-121) was prepared analogously to Int-107 in Example 206, where the di-acid employed in step c was substituted with the step-b di-acid product of Example 199. Ion(s) found by LCMS: [(M+5H)/5]+=539.3, [(M+6H)/6]+=449.6, [(M+7H)/7]+=385.5.

### Example 232—Synthesis of Int-122

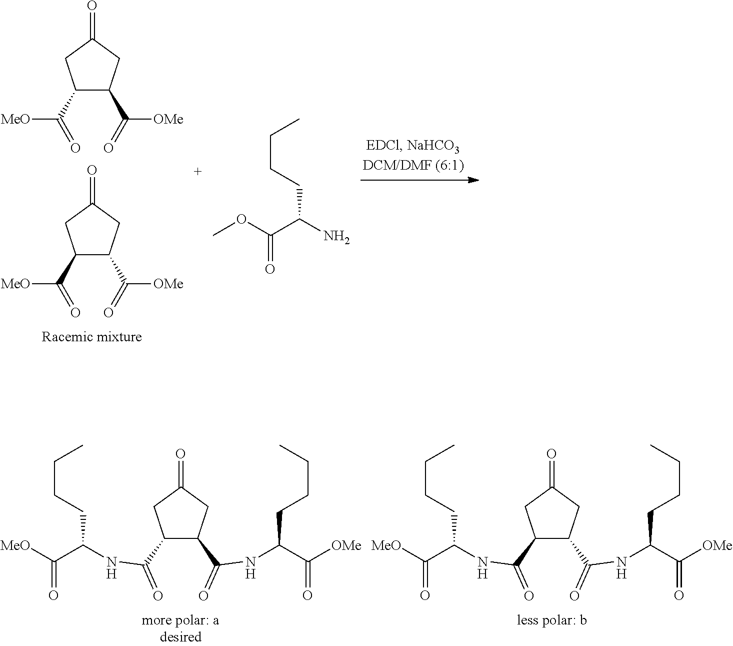

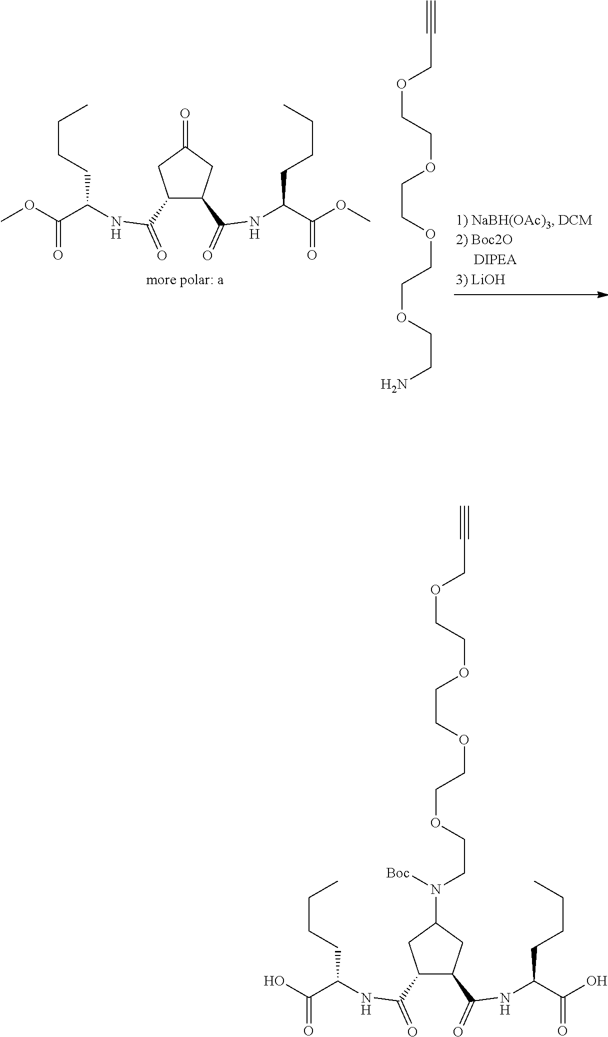

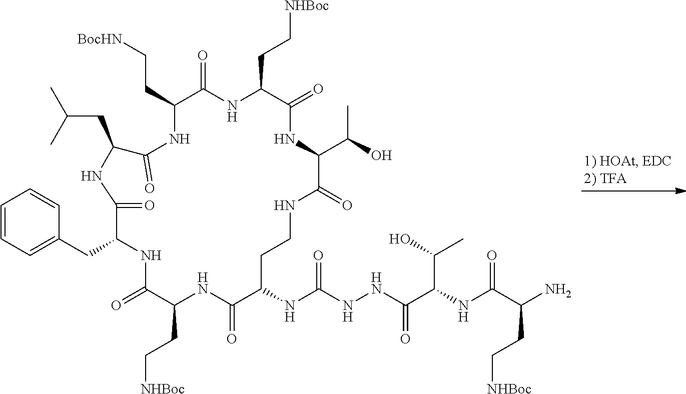

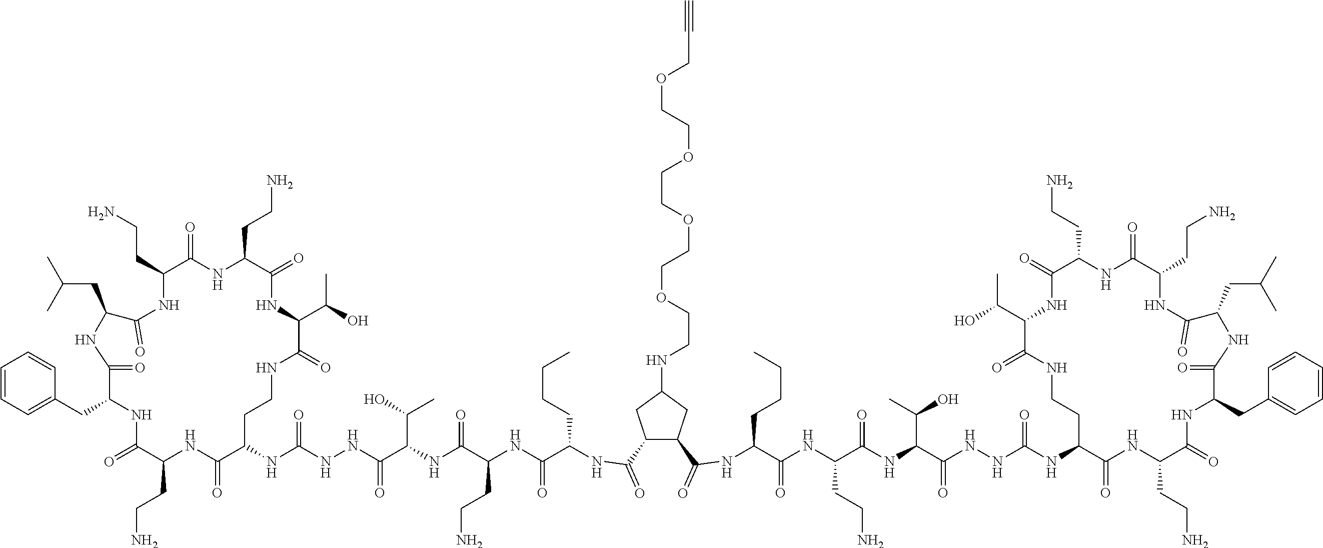

### Step a. Synthesis of Propargyl-PEG4 Central Liner

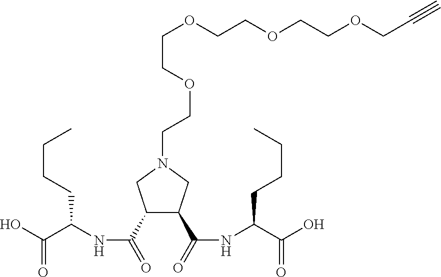 [SMILES: C#CCOCCOCCOCCOCCN1C[C@@H](C(=O)N[C@@H](CCCC)C(=O)O)[C@H](C(=O)N[C@@H](CCCC)C(=O)O)C1]

Racemic-trans-4-oxo-cyclopentane-1,2-dicarboxylic acid (2.369 g, 13.76 mmol), H-norleu-OMe-hydrochloride (5.000 g, 27.52 mmol), and sodium bicarbonate (4.625 g, 55.05 mmol) were dissolved in DCM/DMF (5:1, 30 mL) followed by addition of EDCI (6.596 g, 34.01 mmol) and HOAt (4.683 g, 34.01 mmol). The mixture was stirred for 5 hours then most of the DCM was removed and the residue was precipitated out in 0.5 N aq. HCl (300 mL). The dried solid was subjected to reversed phase liquid chromatography (RPLC) using an Isco Combiflash liquid chromatograph eluted with 30% to 60% acetonitrile and water using 0.1% TFA modifier. The two diastereomers were separated, pooled and lyophilized separately into the more polar diastereomer (a) and the less polar diastereomer (b):Ions found by LC/MS: positive ionization: [M+H⁺]=427.2; negative ionization: [M−H⁺]=425.2 for both intermediates. Yield: 77%. The desired diastereomer (a) was isolated at yield of 42% (based on 50% maximum yield). The stereochemistry of the polar diastereomer was assigned using single crystal x-ray crystallography.

### Step b

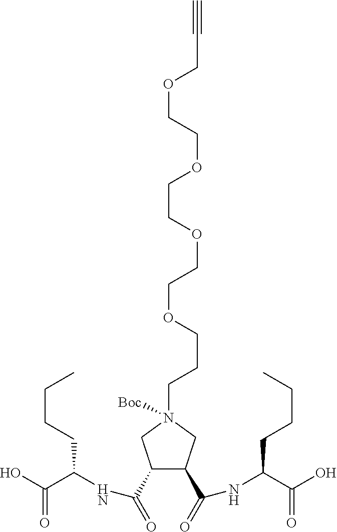

Above polar dimethyl ester (2.450 g, 5.744 mmol), propargyl-PEG4-amine (1.727 g, 7.468 mmol) and acetic acid (0.66 ml, 11.5 mmol) were dissolved in 20 mL of DCM. The mixture was stirred for 1 hour at room temperature followed by addition of sodium triacetatoxyborohydride (2.435 g, 11.49 mmol). The reaction mixture was stirred for overnight (˜15 hours) at room temperature. HPLC analysis showed the completion of the reaction. Ions found by LCMS: M+H⁺=642.4; negative ionization: M−H⁺=640.3. Reaction solvents were removed by rotovap to give a viscous oily residue. The residue was dissolved in 20 mL of THF/10% NaHCO₃ (1:1) and treated with Boc anhydride (2.63 mL, 11.5 mmol) and diisopropylethylamine (2.00 mL, 11.5 mmol). The mixture was stirred for overnight (˜15 hours) at room temperature. Ions found by LCMS, positive ionization: M+H⁺=642.4; negative ionization: M−H⁺=640.3. Most of THF was removed and the residue was extracted with EtOAc. After removal of EtOAc, the residue was dissolved in minimum amount of NMP and purified by reversed phase liquid chromatography (RPLC) using an Isco CombiFlash liquid chromatograph eluted with 30-60% acetonitrile and water. The pure PEG4-amino cyclopentyl dimethyl ester, 3.65 g, was obtained in 86% yield.

The dimethyl ester (3.65 g, 04.920 mmol) was treated with LiOH (0.304 g, 12.3 mmol) in 30 mL of THF/water (1:1), stirred for 1 hr, then acidified with 1N HCl. After removal of THF and extraction with EtOAc, the desired Boc-PEG4-central linker was obtained (3.14 g, 89%). Positive mass ions was found as (M−Boc+H⁺)=615.3, (M+H⁺)=714.4, tr=2.20 minutes with 5-minute 4-95% ACN/water method.

### Step c

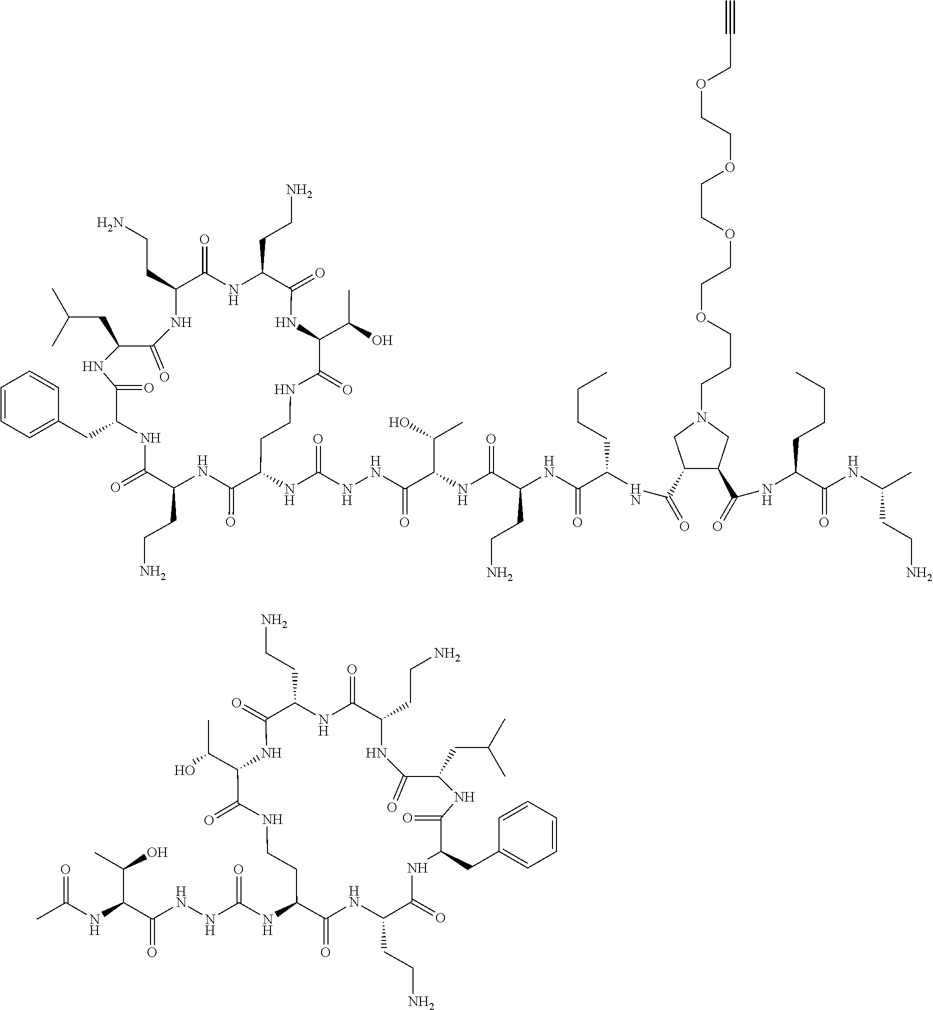 [SMILES: C#CCOCCOCCOCCOCCCN1C[C@@H](C(=O)N[C@@H](CCCC)C(=O)N[C@H](C)CCN)[C@H](C(=O)N[C@@H](CCCC)C(=O)N[C@@H](CCN)C(=O)N[C@H](C(=O)NNC(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)C1 ; CC(=O)N[C@H](C(=O)NNC(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O]

PMBD(aza-gly)-thr-NH2 (1.000 g, 0.703 mmol, see Example 230), central linker diacid from Step a (0.251 g, 0.351 mmol), EDCI (0.169 g, 0.879 mmol), HOAt (0.120 g, 0.879 mmol) and DIPEA (0.25 mL, 1.41 mmol) were mixed. The reaction solution was stirred overnight. DMF was removed by rotary evaporation. The resulting oil was triturated with EtOH. Sonication, filtration and EtOH wash followed by drying to give 1.05 g white solid, 85% mass recovery. Ion(s) found by LCMS: (M−3 Boc+3H⁺)/3=1074.4. The white solid was treated with 30 mL of TFA for 30 min followed by removal of excess TFA. The residue was applied to reversed phase liquid chromatography (RPLC) using an Isco CombiFlash liquid chromatograph eluted with 5% to 45% acetonitrile and water using 0.1% TFA modifier to give the desired product as TFA salt, after lyophilization. Yield 90%. Ion(s) found by LCMS: (M+2H⁺)/2=1310.2, (M+3H⁺)/3=874.2, (M+4H⁺)/4=655.8, (M+5H⁺)/5=525.0, (M+6H⁺)/6=437.6, (M+7H⁺)/7=375.4.

### Example 233—Synthesis of Int-123

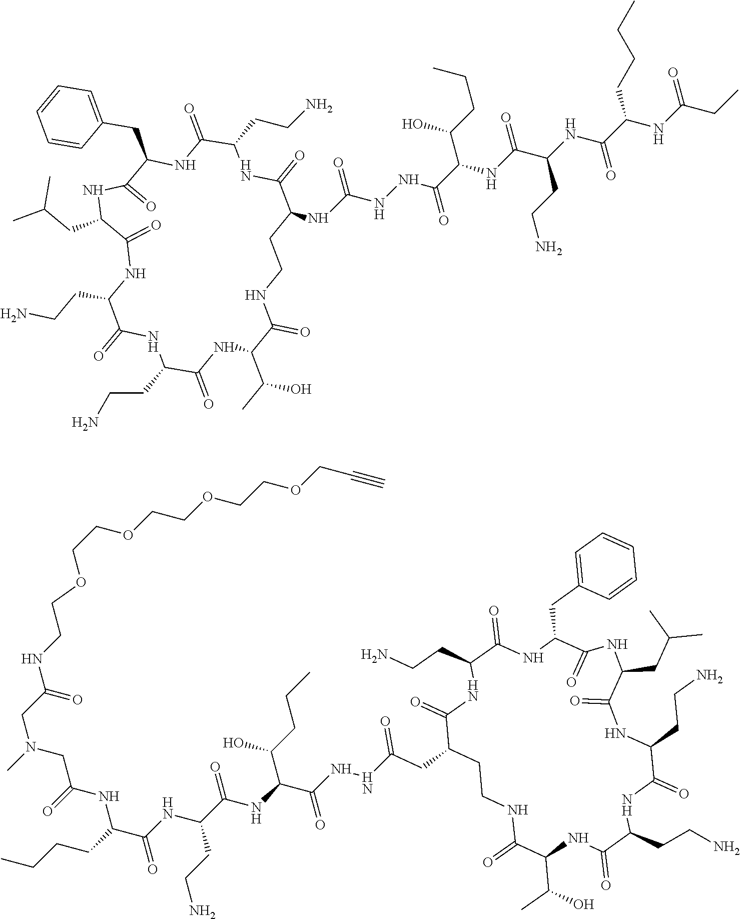

The title compound was prepared analogously to Int-121, where the C2-extended threonine was prepared using the general procedure described in David Aiker, Giles Hamblett, Laurence M. Harwood, Sarah M. Robertson, David J. Watkin and Eleri Williams, Tetrahedron 54, 6089-98 (1998). Ion(s) found by LCMS: [(M+5H)/5]+=539.3, [(M+6H)/6]+=449.6, [(M+7H)/7]+=385.5.

### Example 234: Synthesis of Int-124

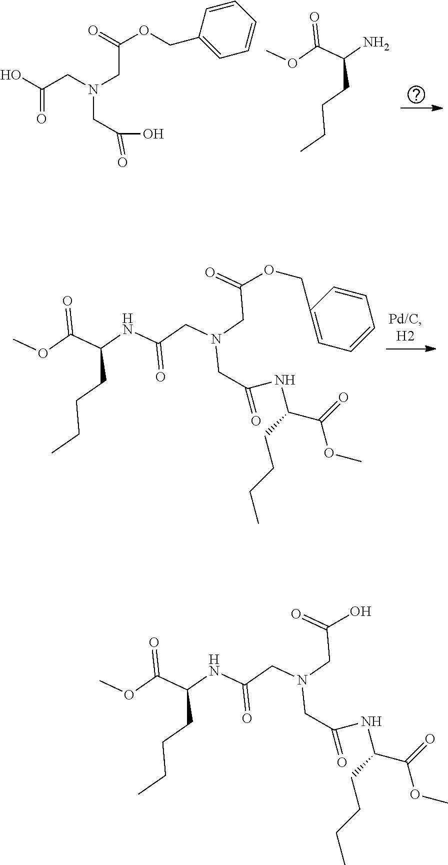

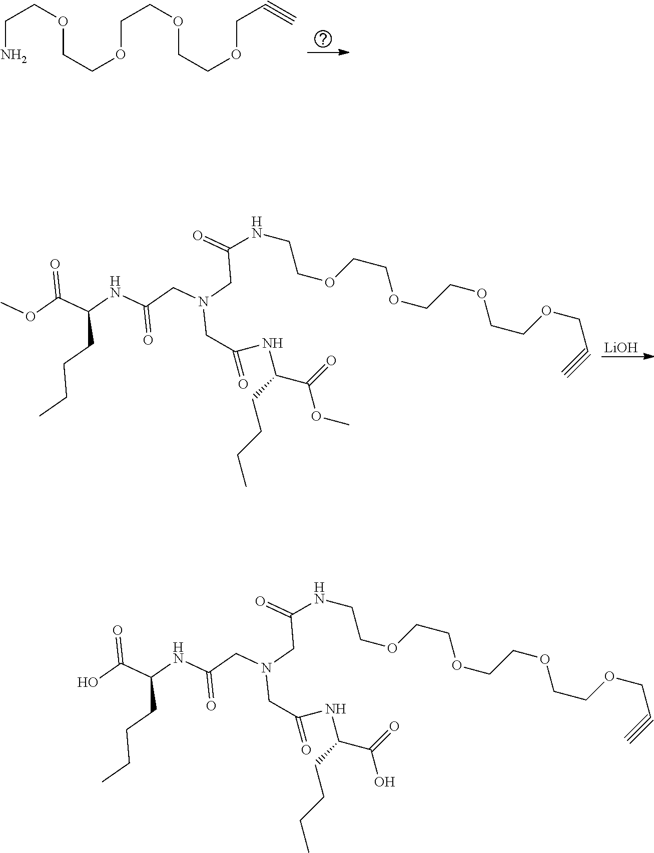

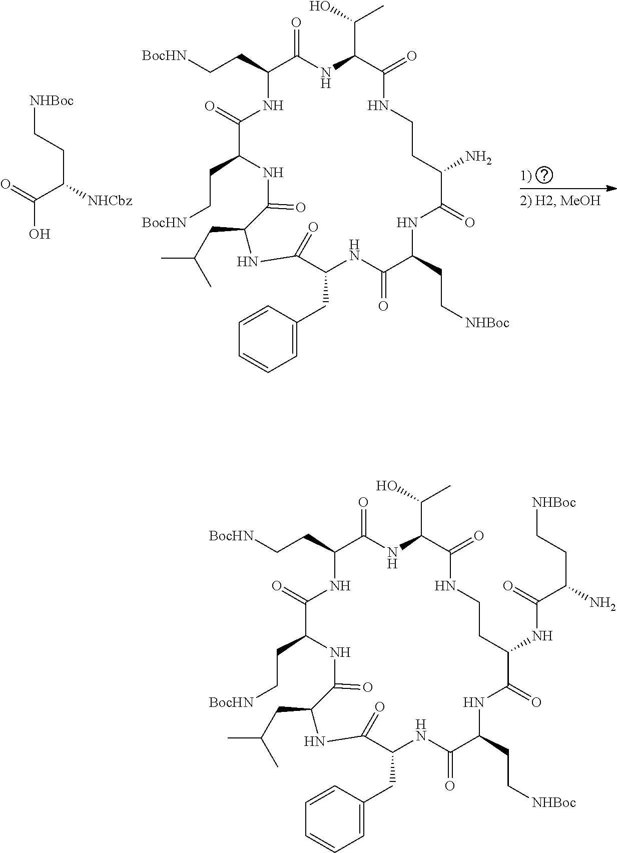 [SMILES: CC(C)(C)OC(=O)NCC[C@H](NC(=O)OCc1ccccc1)C(=O)O ; CC(C)C[C@@H]1NC(=O)[C@@H](Cc2ccccc2)NC(=O)[C@H](CCNC(=O)OC(C)(C)C)NC(=O)[C@@H](N)CCNC(=O)[C@H]([C@@H](C)O)NC(=O)[C@H](CCNC(=O)OC(C)(C)C)NC(=O)[C@H](CCNC(=O)OC(C)(C)C)NC1=O ; CC(C)C[C@@H]1NC(=O)[C@@H](Cc2ccccc2)NC(=O)[C@H](CCNC(=O)OC(C)(C)C)NC(=O)[C@@H](NC(=O)[C@@H](N)CCNC(=O)OC(C)(C)C)CCNC(=O)[C@H]([C@@H](C)O)NC(=O)[C@H](CCNC(=O)OC(C)(C)C)NC(=O)[C@H](CCNC(=O)OC(C)(C)C)NC1=O]

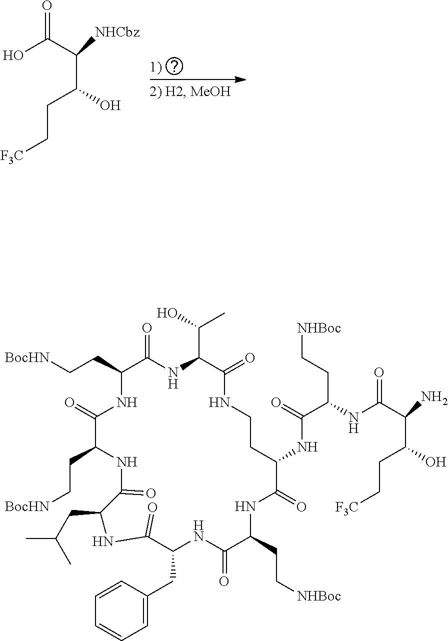 [SMILES: O=C(N[C@H](C(=O)O)[C@H](O)CCC(F)(F)F)OCc1ccccc1 ; CC(C)C[C@@H]1NC(=O)[C@@H](Cc2ccccc2)NC(=O)[C@H](CCNC(=O)OC(C)(C)C)NC(=O)[C@@H](NC(=O)[C@H](CCNC(=O)OC(C)(C)C)NC(=O)[C@@H](N)[C@H](O)CCC(F)(F)F)CCNC(=O)[C@H]([C@@H](C)O)NC(=O)[C@H](CCNC(=O)OC(C)(C)C)NC(=O)[C@H](CCNC(=O)OC(C)(C)C)NC1=O]

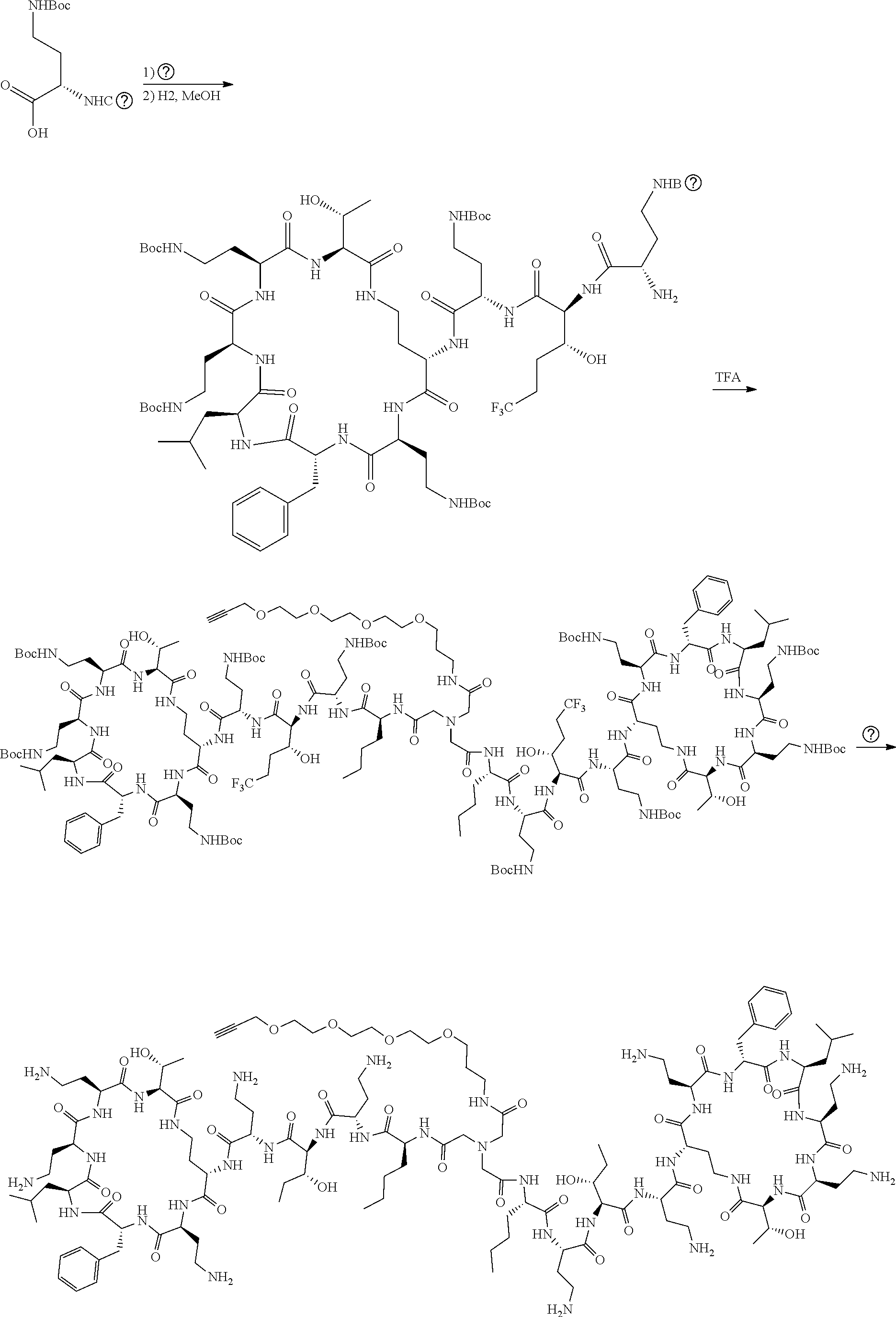

### Step a

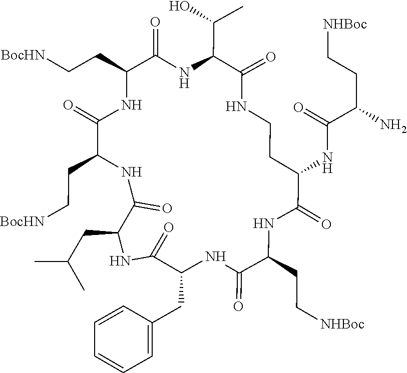 [SMILES: CC(C)C[C@@H]1NC(=O)[C@@H](Cc2ccccc2)NC(=O)[C@H](CCNC(=O)OC(C)(C)C)NC(=O)[C@@H](NC(=O)[C@@H](N)CCNC(=O)OC(C)(C)C)CCNC(=O)[C@H]([C@@H](C)O)NC(=O)[C@H](CCNC(=O)OC(C)(C)C)NC(=O)[C@H](CCNC(=O)OC(C)(C)C)NC1=O]

To a solution of tri-Boc PMB heptapeptide-NH2 (Int-1) (2.1 g, 2 mmol) and (2S)-2-{[(benzyloxy) carbonyl]-amino}-4-[(tert-butoxycarbonyl)amino]butanoic acid (740 mg, 2 mmol) in DMF (30 mL) was added EDC (0.6 g, 3 mmol), HOBT (0.45 g, 3 mmol), and DIEA (0.7 mL, 5 mmol) at room temperature. The solution was stirred overnight. The resulting solution was concentrated, dissolved in 5 mL MeOH, and added drop-wise to 200 mL vigorously stirred water. The resulting heterogenous solution was stirred for one hour, then filtered. The collected filter cake was dissolved into 20 mL methanol, combined with 1 g of 5% Palladium on charcoal, and stirred at room temperature under a hydrogen atmosphere overnight. After the reaction was complete by LCMS, the palladium was removed by filtration through celite. The filtrate was concentrated to yield the title compound (2.2 g, 90%) as white solid, and the crude product was used in the next step without further purification. Ion(s) found by LCMS: [(M+2H)/2]+=631.9, [(M+3H)/3]+=421.6.

### Step b

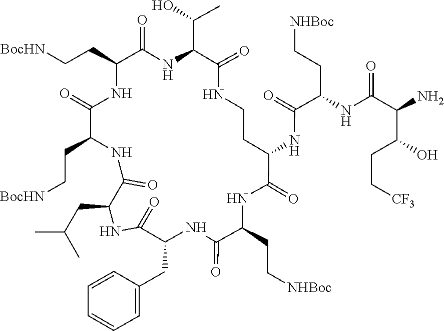 [SMILES: CC(C)C[C@@H]1NC(=O)[C@@H](Cc2ccccc2)NC(=O)[C@H](CCNC(=O)OC(C)(C)C)NC(=O)[C@@H](NC(=O)[C@H](CCNC(=O)OC(C)(C)C)NC(=O)[C@@H](N)[C@H](O)CCC(F)(F)F)CCNC(=O)[C@H]([C@@H](C)O)NC(=O)[C@H](CCNC(=O)OC(C)(C)C)NC(=O)[C@H](CCNC(=O)OC(C)(C)C)NC1=O]

To a solution of step a product (1.2 g, 1 mmol) and 3R-N-[(benzyloxy)carbonyl]-8,6,6-trifluoro-3-hydroxy-L-norleucine (350 mg, 1.05 mmol, prepared as described in David Aiker, Giles Hamblett, Laurence M. Harwood, Sarah M. Robertson, David J. Watkin and Eleri Williams, Tetrahedron 54 (1998)) 6089-6098) in DMF (10 mL) was added EDC (0.3 g, 1.5 mmol), HOBT (0.23 g, 1.5 mmol), and DIEA (0.3 mL, 2 mmol) at room temperature. The reaction was stirred overnight, concentrated, dissolved into 5 mL MeOH, and precipitated by dropwise addition to 100 mL vigorously stirred water. The resulting heterogenous solution was stirred for 1 hour, then filtered. The collected filter cake was dissolved in 20 mL methanol, then combined with 1 g of 5% Palladium on charcoal. The mixture was stirred at room temperature under the hydrogen atmosphere overnight. Palladium charcoal was removed by filtration through celite. The filtrate was concentrated to yield the title compound (1.45 g, 95%) as white solid, and the crude products was used for next step without further purification. Ion(s) found by LCMS: [(M+2H)/2]+=723.4, [(M+3H)/3]+=482.6.

### Step c

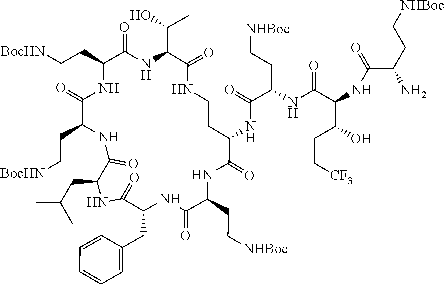 [SMILES: CC(C)C[C@@H]1NC(=O)[C@@H](Cc2ccccc2)NC(=O)[C@H](CCNC(=O)OC(C)(C)C)NC(=O)[C@@H](NC(=O)[C@H](CCNC(=O)OC(C)(C)C)NC(=O)[C@@H](NC(=O)[C@@H](N)CCNC(=O)OC(C)(C)C)[C@H](O)CCC(F)(F)F)CCNC(=O)[C@H]([C@@H](C)O)NC(=O)[C@H](CCNC(=O)OC(C)(C)C)NC(=O)[C@H](CCNC(=O)OC(C)(C)C)NC1=O]

To a solution of the step b product (1.4 g, 1 mmol) and (2S)-2-{[(benzyloxy) carbonyl]-amino}-4-[(tert-butoxycarbonyl)amino]butanoic acid (360 mg, 1 mmol) in DMF (20 mL) was added EDC (0.3 g, 1.5 mmol), HOBT (0.23 g, 1.5 mmol), and Hunigs base (0.3 mL, 2 mmol) at room temperature. The solution was stirred overnight. The resulting solution was concentrated and dissolved into 5 mL MeOH and added dropwise to vigorously stirred water (100 mL), and stirred further for 1 hour, then isolated by filtration. The collected solid was dissolved into 20 mL methanol, then combined with 1 g of 5% palladium on charcoal, and stirred at room temperature under a hydrogen atmosphere overnight. The palladium on charcoal was removed by filtration through celite. The filtrate was concentrated and purified by reversed phase liquid chromatography (RPLC) using an Isco CombiFlash liquid chromatograph eluted with 10% to 100% acetonitrile and water, using 0.1% TFA as the modifier. The pure fractions were lyophilized to yield products 1.1 g, 67% yield. Ion(s) found by LCMS: [(M+2H)/2]+=823.4, [(M+3H)/3]+=549.3, [(M+4H)/4]+=412.2.

### Step d

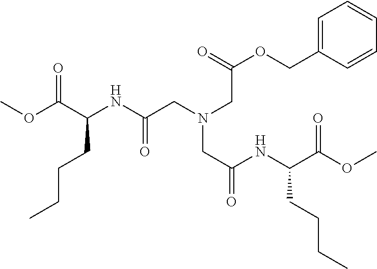 [SMILES: CCCC[C@H](NC(=O)CN(CC(=O)N[C@@H](CCCC)C(=O)OC)CC(=O)OCc1ccccc1)C(=O)OC]

To a solution of step c product (2 g, 7 mmol), and methyl L-norleucinate (2.1 g, 14.2 mmol) in DMF (40 mL) was added EDC (4.0 g, 20 mmol), HOBT (3.1 g, 20 mmol), and DIEA (4.2 mL, 30 mmol) at room temperature. The solution was stirred overnight, then concentrated and purified by reversed phase liquid chromatography (RPLC) using an Isco CombiFlash liquid chromatograph eluted with 5% to 100% acetonitrile and water, using 0.1% TFA as the modifier. The pure fraction was concentrated to yield product 2.7 g, 73% yield. Ion(s) found by LCMS: (M+H)⁺=536.3.

### Step e

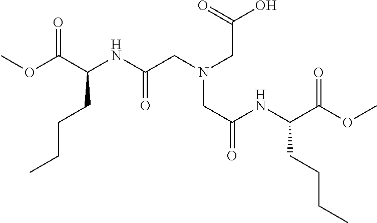

Compound from step d (536 mg, 1 mmol) dissolved in 10 mL of methanol, was treated with 200 mg of 5% palladium on charcoal then stirred at room temperature under the hydrogen atmosphere overnight. The palladium on charcoal was filtered through celite, concentrated to give quantitative yield, and the products was used for next step without further purification.

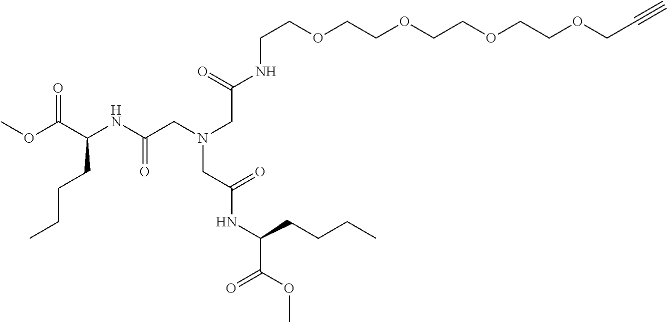 [SMILES: C#CCOCCOCCOCCOCCNC(=O)CN(CC(=O)N[C@@H](CCCC)C(=O)OC)CC(=O)N[C@@H](CCCC)C(=O)OC]

### Step f

A solution of carboxylic acid from step e (450 mg, 1 mmol), propargyl-PEG4-NH2 (230 mg, 1 mmol) dissolved in DMF (5 mL) was treated with EDC (300 mg, 1.5 mmol), HOBT (230 mg, 1.5 mmol), and DIEA (0.3 mL, 1.5 mmol) at room temperature. The solution was stirred overnight, concentrated and purified by reversed phase liquid chromatography (RPLC) using an Isco CombiFlash liquid chromatograph eluted with 5% to 100% acetonitrile and water, using 0.1% TFA as the modifier. The pure fractions were concentrated giving 650 mg, 93% yield. Ion(s) found by LCMS: (M+H)+=659.4.

### Step g

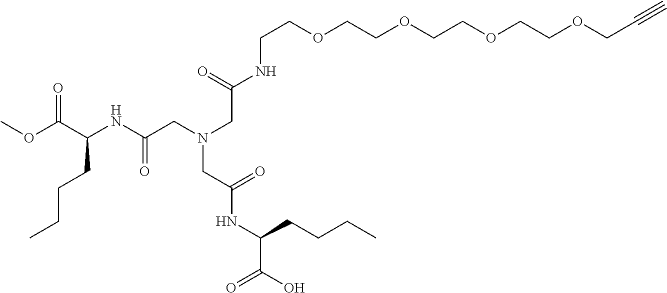 [SMILES: C#CCOCCOCCOCCOCCNC(=O)CN(CC(=O)N[C@@H](CCCC)C(=O)O)CC(=O)N[C@@H](CCCC)C(=O)OC]

Lithium hydroxide (120 mg, 5 mmol) in 2 mL water was added to a solution of di-ester from step f (0.65 g, 1 mmol) dissolved in 2 mL MeOH and 2 mL THF. After consumption of starting material as determined by LCMS, the solution was acidified with Amberlite® IRN-77, ion exchange resin to a pH=1. The solution was filtered, concentrated to give quantitative yield of desired product, which was used in next step without further purification. Ion(s) found by LCMS: (M+H)⁺=631.4.

### Step h

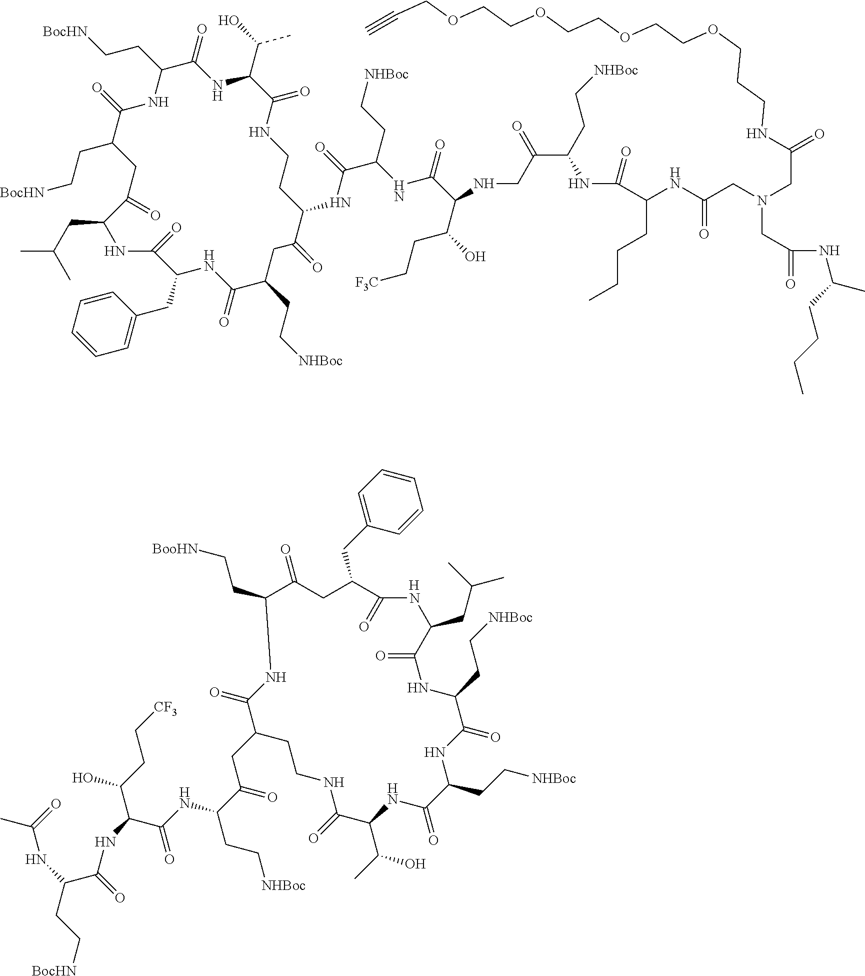

A solution of the compound from step g (56 mg, 0.089 mmol), the product from Step-c above (295 mg, 0.18 mmol, 2.1 eq) dissolved in DMF (5 mL) was treated with EDC (60 mg, 0.3 mmol), HOBT (45 mg, 0.3 mmol), and DIEA (0.14 mL, 1.0 mmol) at room temperature. The solution was stirred overnight, concentrated, dissolved in 2 mL MeOH, and precipitated by adding dropwise to 50 mL vigorously stirred water. The filter cake was used in the next step without additional purification.

### Step i

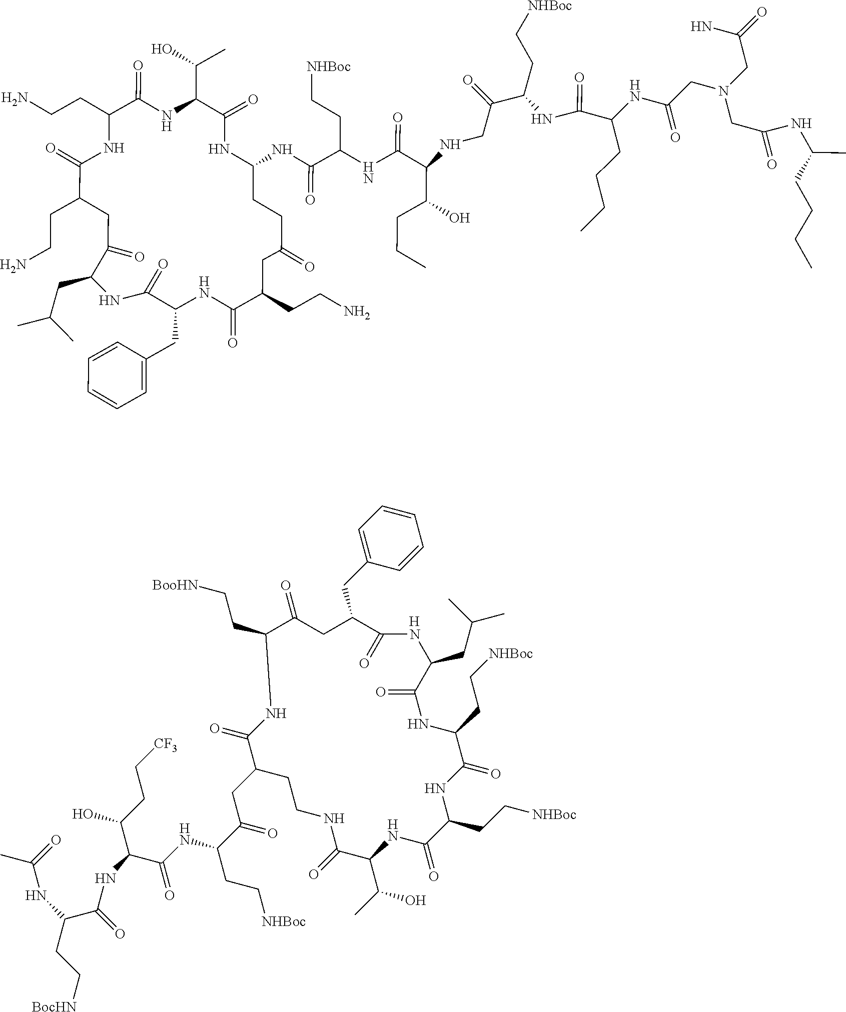

Product from step h was dissolved in 2 mL DCM and 2 mL TFA at room temperature and stirred 10 min, then concentrated and purified by reversed phase liquid chromatography (RPLC) using an Isco CombiFlash liquid chromatograph eluted with 0% to 100% acetonitrile and water, using formic acid as the modifier. Yield 105 mg, two steps, 38% yield. Ion(s) found by LCMS: [(M+5H)/5]+=577.7, [(M+6H)/6]+=481.6, [(M+7H)/7]+=412.9.

### Example 235—Synthesis of Int-125

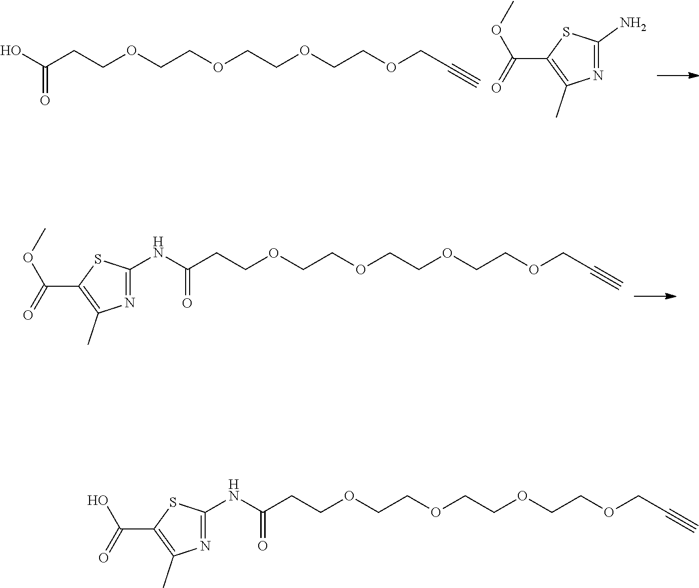

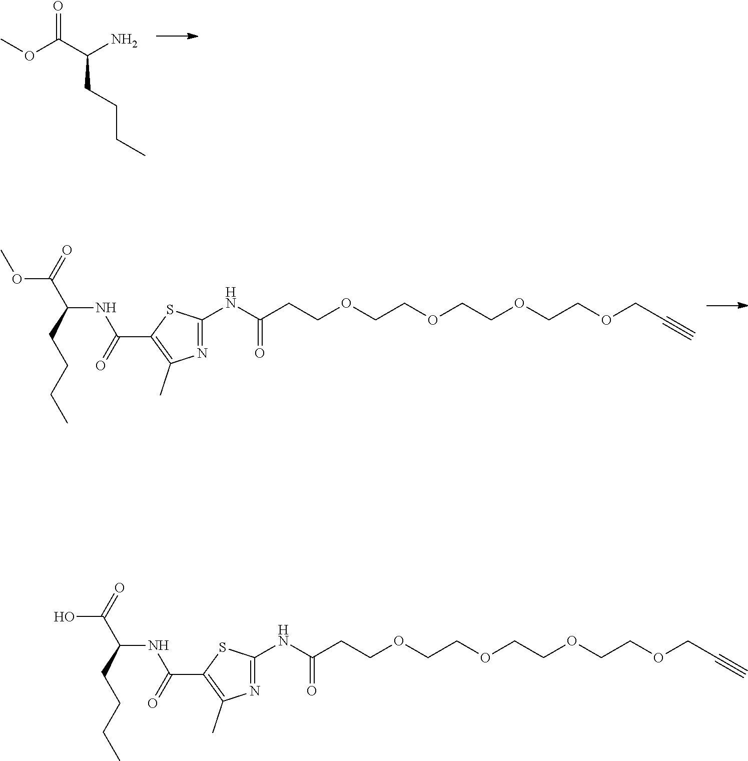

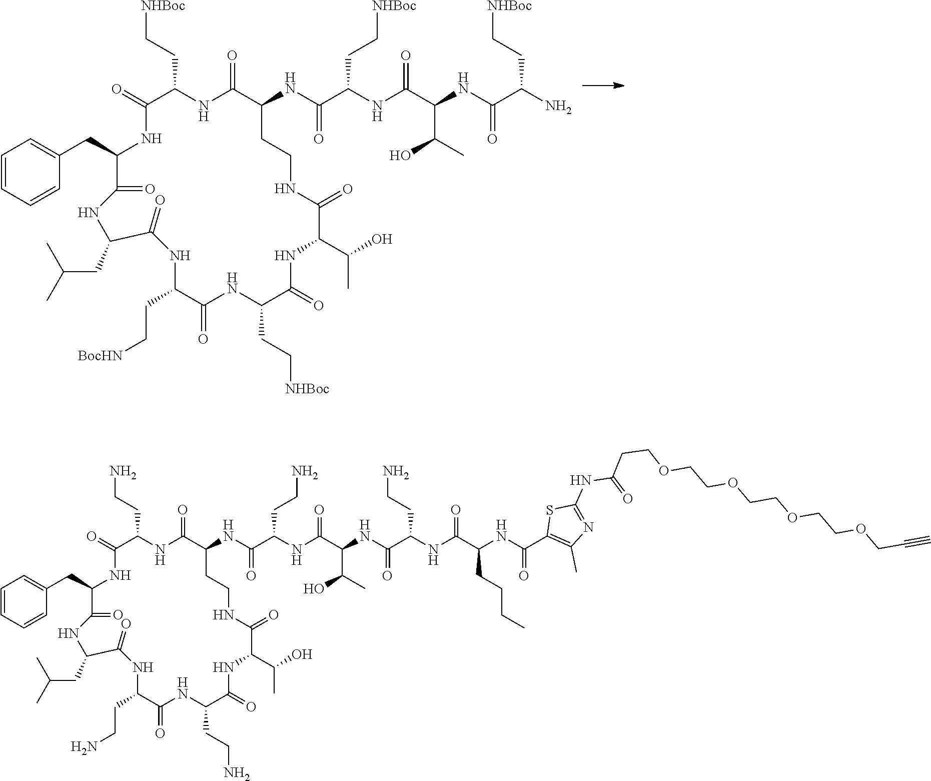 [SMILES: CC(C)C[C@@H]1NC(=O)[C@@H](Cc2ccccc2)NC(=O)[C@H](CCNC(=O)OC(C)(C)C)NC(=O)[C@@H](NC(=O)[C@H](CCNC(=O)OC(C)(C)C)NC(=O)[C@@H](NC(=O)[C@@H](N)CCNC(=O)OC(C)(C)C)[C@@H](C)O)CCNC(=O)[C@H]([C@@H](C)O)NC(=O)[C@H](CCNC(=O)OC(C)(C)C)NC(=O)[C@H](CCNC(=O)OC(C)(C)C)NC1=O ; C#CCOCCOCCOCCOCCC(=O)Nc1nc(C)c(C(=O)N[C@@H](CCCC)C(=O)N[C@@H](CCN)C(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)s1]

### Step a

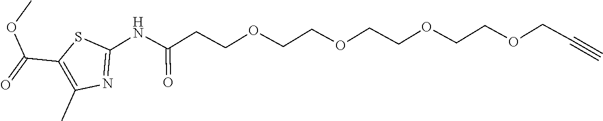 [SMILES: C#CCOCCOCCOCCOCCC(=O)Nc1nc(C)c(C(=O)OC)s1]

Methyl 2-amino-4-methyl-1,3-thiazole-5-carboxylate (0.41 g, 2.31 mmol), and acid-PEG4-alkyne (0.50 g, 1.92 mmol), HOAt (1.31 g, 5.76 mmol), EDCI (1.10 g, 5.76 mmol) and DIEA (2.1 mL, 11.53 mmol) were stirred in anhydrous DMF (8 mL) at mom temperature overnight. Next day, DMF solvent was reduced under reduced pressure then was purified by reversed phase liquid chromatography (RPLC) using Isco CombiFlash liquid chromatography eluted with 5% to 50% acetonitrile and water, using 0.1% TFA as modifier. Yield of product was 0.37 g, 47%. Ion(s) found by LCMS: (M−Boc+H)⁺=424.3

### Step b

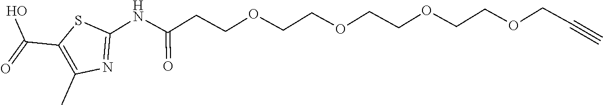 [SMILES: C#CCOCCOCCOCCOCCC(=O)Nc1nc(C)c(C(=O)O)s1]

A solution of the methyl ester from the previous step (0.37 g, 0.09 mmol) and LiOH (0.026 g, 1.10 mmol) were stirred at room temperature in MeOH:THF:H₂O (1:1:2, 12 mL) overnight. The reaction was monitored by analytical HPLC. After day 1, the ratio of was desired product to starting material was 1:5. Another portion of LiOH (0.026 mg, 1.10 mmol) was added then it was stirred for another day. Day 2 showed the reaction conversion's rate was 1:1 ratio of P:SM. NaOH (0.074 g, 1.29 mmol) was added and stirred for several days. The reaction was acidified with Amberlite®IRN-77 then filtered and washed with MeOH. Yield of product was 0.24 g, 66%. Ion(s) found by LCMS: (M+H)+=401.2

### Step c

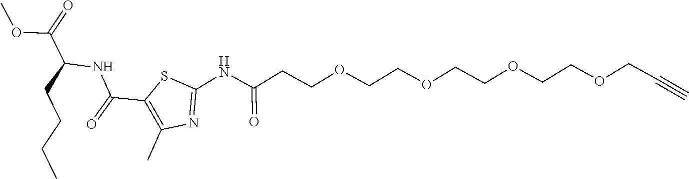 [SMILES: C#CCOCCOCCOCCOCCC(=O)Nc1nc(C)c(C(=O)N[C@@H](CCCC)C(=O)OC)s1]

A solution of the acid product from the previous step (0.24 g, 0.59 mmol), HOAt (0.24 g, 1.78 mmol), EDCI (0.34 g, 1.78 mmol) and DIEA (0.62 mL, 3.57 mmol) in DMF (3 mL) was stirred at room temperature for 15 min then methyl (2S)-2-aminohexanoate (0.13 g, 0.71 mmol) was added. The reaction was stirred for 16 hours. The reaction mixture was purified by reversed phase liquid chromatography (RPLC) using Isco CombiFlash liquid chromatography eluted with 5% to 50% acetonitrile and water, using 0.1% TFA as modifier. Yield of product was 0.13 g, 40%. Ion(s) found by LCMS: (M+H)+=528.2

### Step d

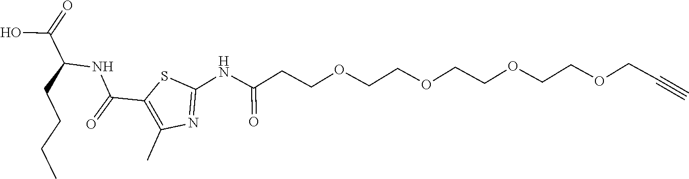 [SMILES: C#CCOCCOCCOCCOCCC(=O)Nc1nc(C)c(C(=O)N[C@@H](CCCC)C(=O)O)s1]

A solution of methyl ester from a previous step (0.13 g, 0.24 mmol) and LiOH (8.6 mg, 0.36 mmol) was stirred in MeOH:THF:H₂O (1:1:2, 8 mL) at room temperature for 2 hours then was acidified with Amberlite®IRN-77. The reaction mixture was filtered and washed with methanol. The filtrate was concentrated to give the desired product. Yield of product was 52 mg, 42%. Ion(s) found by LCMS: (M+H)+=514.2

### Step e

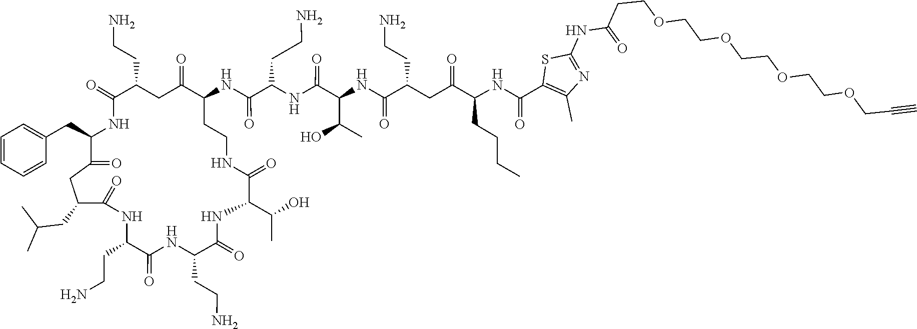

A solution of the product from the previous step (0.056 g, 0.11 mmol), Int-7a (0.14 g, 0.088 mmol, HOAt (0.037 g, 0.26 mmol), and EDCI (0.05 g, 0.26 mmol) were dissolved in DMF (2 mL) then charged with DIEA (0.093 mL, 0.53 mmol). The reaction was stirred at room temperature overnight then purified by reversed phase liquid chromatography (RPLC) using an Isco CombiFlash liquid chromatography eluted with 5% to 80% acetonitrile and water, using 0.1% TFA as modifier to yield the Boc-protected product.

A solution of the deca-boc-protected intermediate from the previous step was stirred in TFA:CH₂Cl₂ (1:1, 6 mL) at ambient temperature for 1 hour then stripped of TFA and CH₂Cl₂ using the rotary evaporator. The residue was purified by reversed phase high pressure liquid chromatography (HPLC) using an Isco ACCQ-Prep liquid chromatography eluted with 0% to 25% acetonitrile and water, using 0.1% TFA as modifier. Yield of product was 0.069 g, 37% for 2 steps. Ion(s) found by LCMS: [(M+2H)/2]+=780.1, [(M+3H)/3]+=520.5, [(M+4H)/4]+=390.5

### Example 236—Synthesis of Int-126

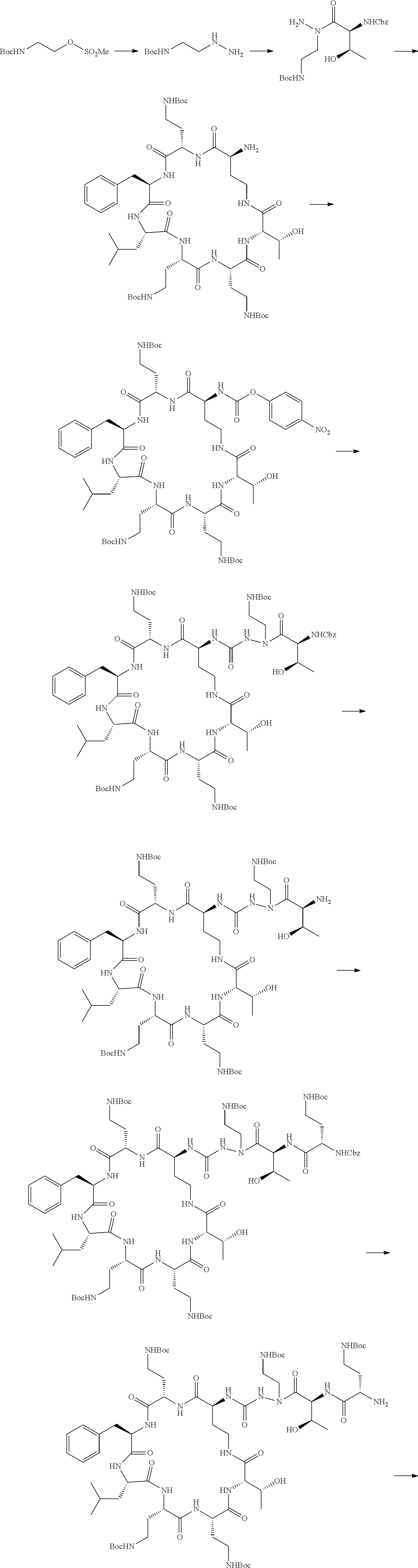

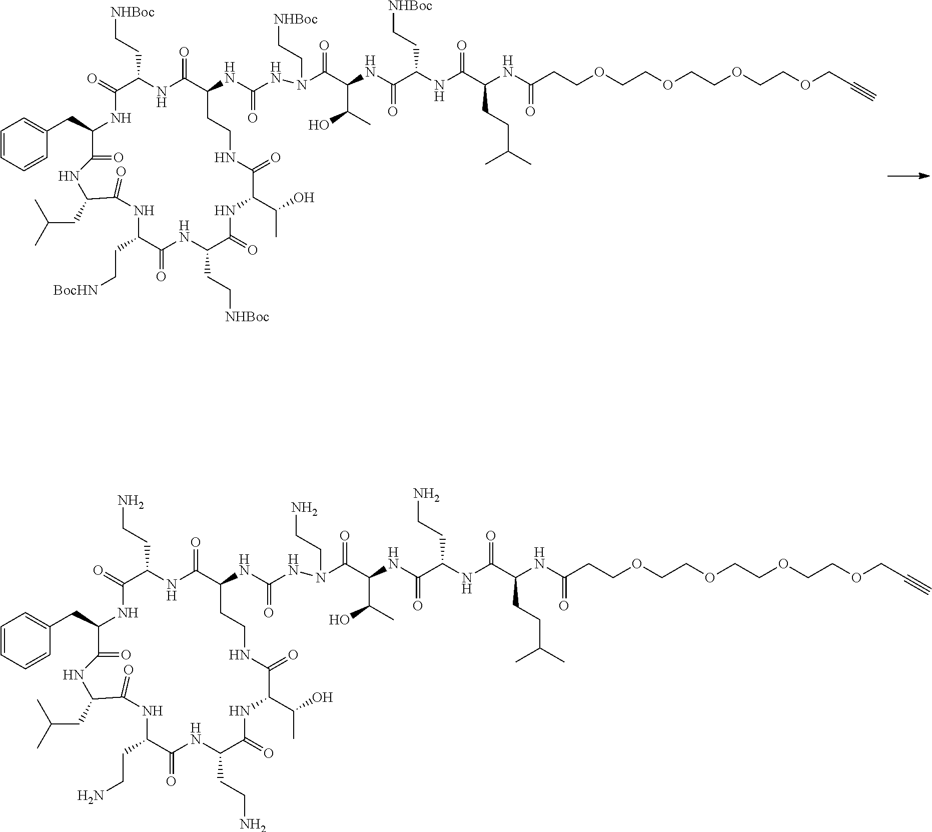

### Step a

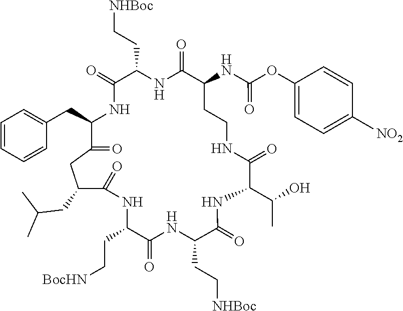 [SMILES: CC(C)C[C@@H]1CC(=O)[C@@H](Cc2ccccc2)NC(=O)[C@H](CCNC(=O)OC(C)(C)C)NC(=O)[C@@H](NC(=O)Oc2ccc([N+](=O)[O-])cc2)CCNC(=O)[C@H]([C@@H](C)O)NC(=O)[C@H](CCNC(=O)OC(C)(C)C)NC(=O)[C@H](CCNC(=O)OC(C)(C)C)NC1=O]

Int-1 (5.315 g, 5 mmol) was slowly added to DCM (50 ml) while stirring, then cooled in an ice-water bath. A solution of sodium bicarbonate (462 mg, 5.5 mmol) in water (10 ml) was added, followed by the addition of 4-nitrophenylchloroformate (1.512 g, 7.5 mmol). The ice-water bath was removed, and the reaction mixture was continued for 1 hour. The product, which turned into gel during the reaction, was filtered and washed with water. It was further dried under high vacuum to a white solid. The crude product was carried to the subsequent step without further purification. Yield 6.14 g, ˜95% pure. Ion found by LCMS: [M-Boc+H]⁺=1127.1, [(M−2Boc−tbu+2H)/2]⁺=486.2, [(M−3Boc+2H)/2]⁺=464.2.

### Step b

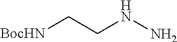 [SMILES: CC(C)(C)OC(=O)NCCNN]

To a solution of tert-butyl N-[2-(methanesulfonyl)ethyl]carbamate (6.31 g, 20 mmol) in anhydrous EtOH (10 ml) was added hydrazine (2.56 g, 80 mmol). The resulting mixture was heated at 80° C. for 1.5 hours, then concentrated by rotary evaporation. The residue was purified by RPLC (150 g SiO2, 0% to 40% acetonitrile and water). Yield 85%, 2.98 g. Ion found by LCMS: [M+H]⁺=176.

### Step c

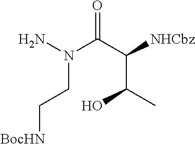 [SMILES: C[C@@H](O)[C@H](NC(=O)OCc1ccccc1)C(=O)N(N)CCNC(=O)OC(C)(C)C]

A mixture of the step-b product (486 mg, 2.77 mmol), Z-Thr-OH (605 mg, 3.39 mmol) and HOBT was dissolved in anhydrous DMF (2.5 mL). EDC HCl (512 mg, 2.68 mmol) was added, and the reaction was stirred for 30 minutes. It was then directly purified through RPLC (100 g SiO2, 5% to 47% acetonitrile and water). Yield 1.25 g, 89.8%. Ion found by LCMS: [M+H]⁺=411.0, [M-Boc+H]⁺=311.0.

### Step d

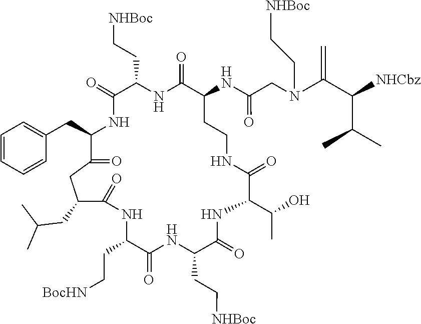

A mixture of the step-c-product (177 mg, 0.432 mmol) and the step-a product (640 mg, 0.52 mmol) was dissolved in anhydrous DMF (1.5 mL). After 1 hour, DIPEA (30 mg, 0.22 mmol) was added and the reaction was continued at room temperature to 55° C. overnight. It was then directly purified by RPLC (100 g SiO2, 40% to 90% MeOH and water). Yield 245.8 mg, 38%. Ions found by LCMS: [(M−2Boc+2H)/2]⁺=650.0, [(M−2Boc-tBu+2H)/2]⁺=621.8, [(M−3Boc+2H)/2]⁺=600.0, [(M−4Boc+2H)/2]⁺=550.0.

### Step e

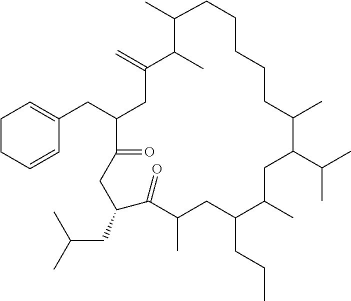 [SMILES: C=C1CC(CC2=CCCC=C2)C(=O)C[C@@H](CC(C)C)C(=O)C(C)CC(CCC)C(C)CC(C(C)C)C(C)CCCCCC(C)C1C]

To the step-d product (245.8 mg, 0.164 mmol) in MeOH (20 mL) was added 4N HCl solution in dioxane (0.05 mL) and a solution of Pd/C (dry basis, 200 mg) in water (0.3 mL) and MeOH (1 mL). The mixture was stirred under hydrogen atmosphere for 1 hour. Pd/C was filtered, and the filtrate was concentrated at room temperature. The residue was purified through RPLC (100 g SiO2, 40% to 80% MeOH and water). Yield 177.7 mg, 79.4%. Ions found by LCMS: [(M−Boc+2H)/2]⁺=632.8, [(M−Boc−tBu+2H)/2]⁺=604.8, [(M−2Boc+2H)/2]⁺=582.8, [(M−2Boc-tBu+2H)/2]⁺=555.0, [(M−3Boc+2H)/2]⁺=532.8, [(M−4Boc+2H)/2]⁺=482.8.

### Step f

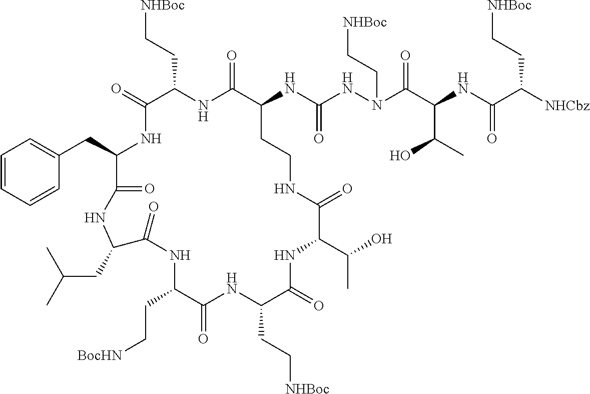 [SMILES: CC(C)C[C@@H]1NC(=O)[C@@H](Cc2ccccc2)NC(=O)[C@H](CCNC(=O)OC(C)(C)C)NC(=O)[C@@H](NC(=O)NN(CCNC(=O)OC(C)(C)C)C(=O)[C@@H](NC(=O)[C@H](CCNC(=O)OC(C)(C)C)NC(=O)OCc2ccccc2)[C@@H](C)O)CCNC(=O)[C@H]([C@@H](C)O)NC(=O)[C@H](CCNC(=O)OC(C)(C)C)NC(=O)[C@H](CCNC(=O)OC(C)(C)C)NC1=O]

To a mixture of Z-Dab-OH (70.5 mg, 0.2 mmol) and HATU (76 mg, 0.2 mmol) in anhydrous DMF (0.5 mL) was added DIEPA (39 mg, 0.3 mmol). After 5 minutes, the step-e product (175 mg, 0.119 mmol) was added, and the reaction mixture was stirred for 30 minutes. It was then directly purified by RPLC (100 g SiO2, 50% to 90% MeOH and water). Yield 177.5 mg, 87.4%. Ions found by LCMS: [(M−2Boc+2H)/2]⁺=749.8, [(M−2Boc-tBu+2H)/2]⁺=721.8, [(M−4Boc+2H)/2]⁺=650.0.

### Step g

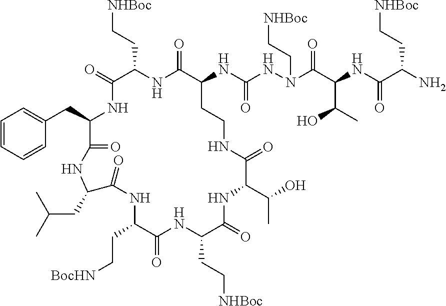 [SMILES: CC(C)C[C@@H]1NC(=O)[C@@H](Cc2ccccc2)NC(=O)[C@H](CCNC(=O)OC(C)(C)C)NC(=O)[C@@H](NC(=O)NN(CCNC(=O)OC(C)(C)C)C(=O)[C@@H](NC(=O)[C@@H](N)CCNC(=O)OC(C)(C)C)[C@@H](C)O)CCNC(=O)[C@H]([C@@H](C)O)NC(=O)[C@H](CCNC(=O)OC(C)(C)C)NC(=O)[C@H](CCNC(=O)OC(C)(C)C)NC1=O]

To the step-f product (177.5 mg, 0.104 mmol) in MeOH (10 mL) was added 4N HCl solution in dioxane (0.025 mL) and a solution of Pd/C (dry basis, 100 mg) in water (0.3 mL):MeOH (1 mL). The mixture was stirred under hydrogen for 1 hour. Pd/C, filtered, and concentrated at room temperature. The residue was purified by RPLC (100 g SiO2, 50% to 90% MeOH and water). Yield 156.4 mg, 96.2%. Ions found by LCMS: [(M−Boc+2H)/2]⁺=733, [(M−Boc-tBu+2H)/2]⁺=705.0, [(M−2Boc+2H)/2]⁺=683.

### Step h

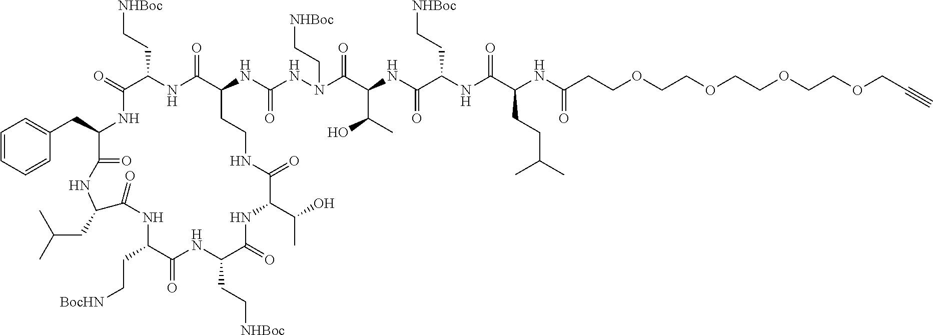 [SMILES: C#CCOCCOCCOCCOCCC(=O)N[C@@H](CCC(C)C)C(=O)N[C@@H](CCNC(=O)OC(C)(C)C)C(=O)N[C@H](C(=O)N(CCNC(=O)OC(C)(C)C)NC(=O)N[C@H]1CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](Cc2ccccc2)NC(=O)[C@H](CCNC(=O)OC(C)(C)C)NC1=O)[C@@H](C)O]

To a mixture of propargyl-PEG4-homoLeu-OH (58 mg, 0.15 mmol, described in Example 226 Int-116) and HATU (62.7 mg, 0.165 mmol) in anhydrous DMF (0.5 mL) was added DIEA (39 mg, 0.3 mmol). After 5 minutes, the step-f product (156.4 mg, 0.1 mmol) was added, and the reaction mixture was stirred for 30 minutes. It was then directly purified by RPLC (100 g SiO2, 50% to 90% MeOH and water). Yield 167.1 mg, 86.4%. Ions found by LCMS: [(M−2Boc+2H)/2]⁺=867.6, [(M−2Boc-tBu+2H)/2]⁺=839.5.

### Step i

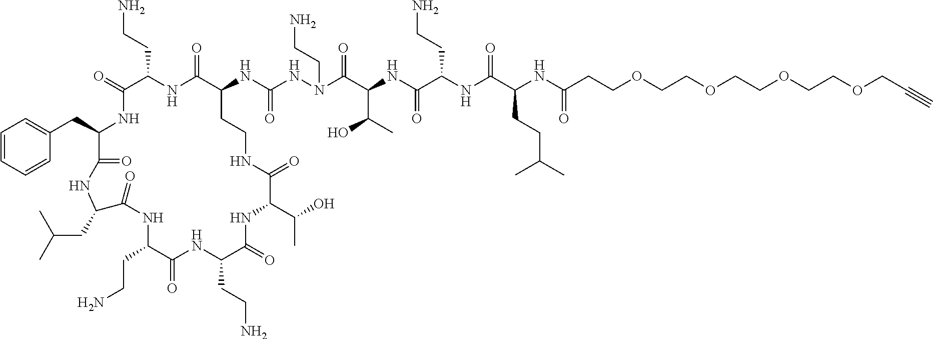 [SMILES: C#CCOCCOCCOCCOCCC(=O)N[C@@H](CCC(C)C)C(=O)N[C@@H](CCN)C(=O)N[C@H](C(=O)N(CCN)NC(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O]

The step-h product (167.1 mg, 0.0864 mmol) was dissolved in TFA/DCM (1:1, 2 mL). The solution was stirred at 30° C. for 20 minutes, then directly purified by HPLC (5% to 30% acetonitrile and water, using 0.1% TFA as modifier). Yield 131.6 mg, 76%. Ions found by LCMS: [(M+2H)/2]⁺=717.6. [(M+3H)/3]⁺=478.8, [(M+4H)/4]⁺=359.4.

### Example 237—Synthesis of Int-127

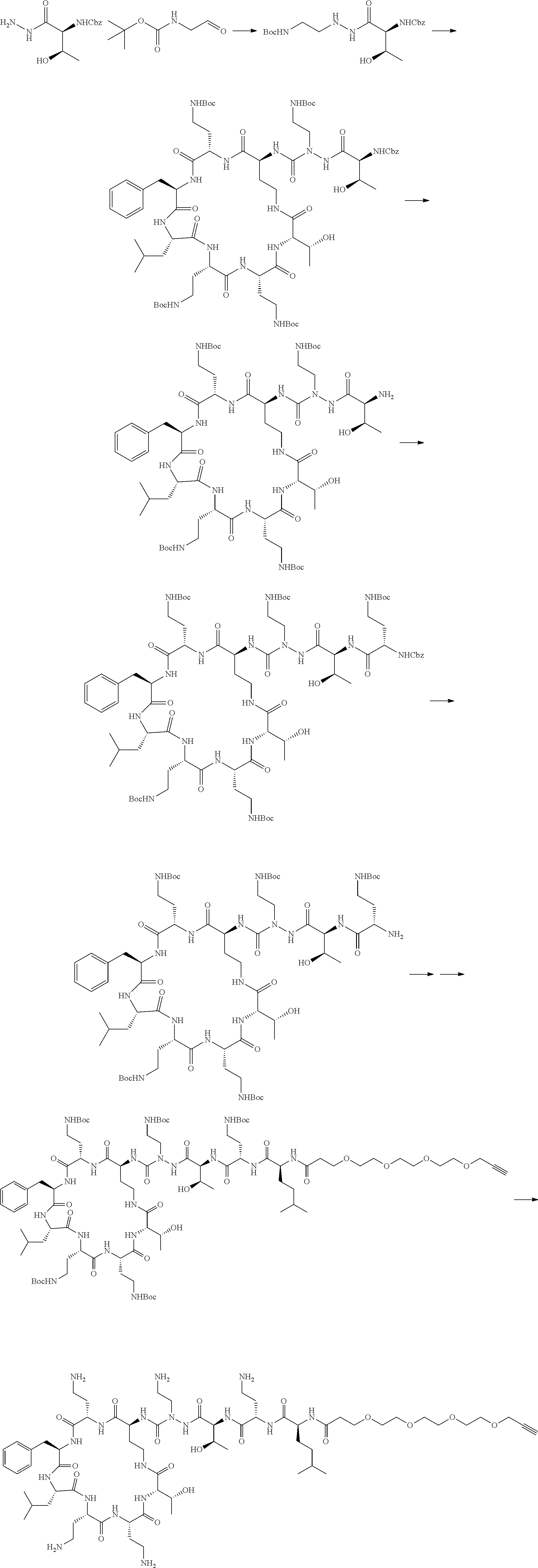

### Step b

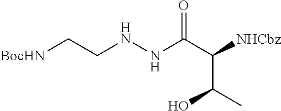 [SMILES: C[C@@H](O)[C@H](NC(=O)OCc1ccccc1)C(=O)NNCCNC(=O)OC(C)(C)C]

To a suspension of (Z)-Thr-hydrazide (1.34 g, 5 mmol) in anhydrous EtOH (20 mL) at 80° C. was added AcOH (60 mg, 1 mmol) and N-Boc-2 aminoacetaldehyde (980 mg, 6.16 mmol). After 1 hour, the resulting mixture was cooled to room temperature and concentrated by rotary evaporation to a yellow foam. The material was re-dissolved in DCM (15 mL), and AcOH (120 mg, 2 mmol) was added, followed by the addition of sodium triacetoxyborohydride (2.7 g, 6 mmol) in portions over 15 minutes. The reaction mixture was stirred at room temperature overnight and monitored by LCMS (additional sodium triacetoxyborohydride may be added if needed). After the reaction was complete, the solid was filtered, and the filtrate concentrated by rotary evaporation. The residue was purified by RPLC (150 g SiO2, 5% to 40% acetonitrile and water). Yield 1.02 g, 48.7%. Ions found by LCMS: [M−H]⁺=411.2, [M-Boc+H]⁺=311.1.

### Step b

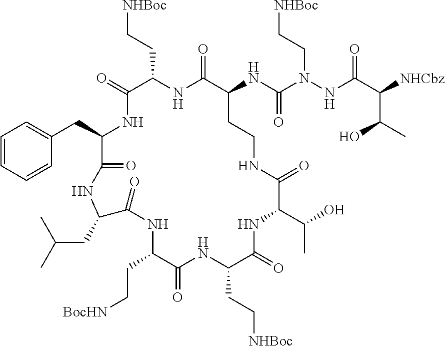 [SMILES: CC(C)C[C@@H]1NC(=O)[C@@H](Cc2ccccc2)NC(=O)[C@H](CCNC(=O)OC(C)(C)C)NC(=O)[C@@H](NC(=O)N(CCNC(=O)OC(C)(C)C)NC(=O)[C@@H](NC(=O)OCc2ccccc2)[C@@H](C)O)CCNC(=O)[C@H]([C@@H](C)O)NC(=O)[C@H](CCNC(=O)OC(C)(C)C)NC(=O)[C@H](CCNC(=O)OC(C)(C)C)NC1=O]

A mixture of the step-a product (572.3, 0.466 mmol, described in Example 236 Int-126) and the product from above (165.3 mg, 0.402 mmol) were dissolved in anhydrous DMF (1.5 mL) and heated at 60° C. for 4 days. The reaction mixture was then directly purified by RPLC (100 g SiO2, 50% to 95% MeOH and water). Yield 430 mg, 71.4%. Ions found by LCMS: [(M−2Boc+2H)/2]⁺=649.8, [(M−2Boc−tBu+2H)/2]⁺=621.8, [(M−3Boc+2H)/2]⁺=599.8, [(M−4Boc+2H)/2]⁺=549.8.

### Step c

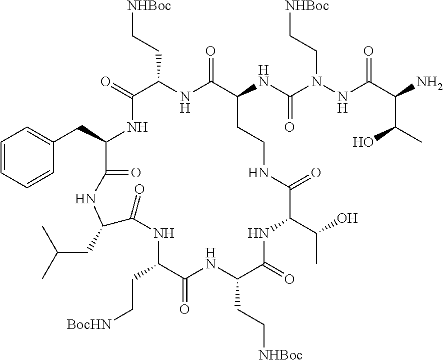 [SMILES: CC(C)C[C@@H]1NC(=O)[C@@H](Cc2ccccc2)NC(=O)[C@H](CCNC(=O)OC(C)(C)C)NC(=O)[C@@H](NC(=O)N(CCNC(=O)OC(C)(C)C)NC(=O)[C@@H](N)[C@@H](C)O)CCNC(=O)[C@H]([C@@H](C)O)NC(=O)[C@H](CCNC(=O)OC(C)(C)C)NC(=O)[C@H](CCNC(=O)OC(C)(C)C)NC1=O]

To the step-b product (396 mg, 0.264 mmol) in MeOH (10 mL) was added 4N HCl solution in dioxane (0.07 mL) and a solution of Pd/C (dry basis, 200 mg) in water (0.3 mL):MeOH (1 mL). The mixture was stirred under hydrogen atmosphere for 1 hour. Pd/C was filtered off, and the filtrate was concentrated at room temperature. The residue was purified by RPLC (100 g SiO2, 50% to 90% MeOH and water). Yield 278.5 mg, 77.3%. Ions found by LCMS: [M+H]⁺=1364.6, [(M−Boc+2H)/2]⁺=633, [(M−Boc-tBu+2H)/2]⁺=604.8, [(M−2Boc+2H)/2]⁺=582.8, [(M−2Boc−tBu+2H)/2]⁺=554.8, [(M−3Boc+2H)/2]⁺=533, [(M−4Boc+2H)/2]⁺=482.8.

### Step d

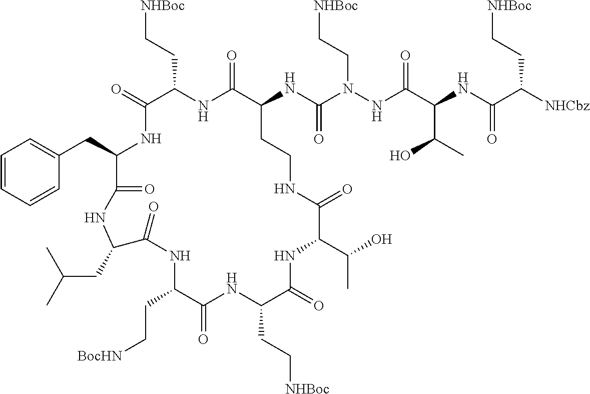 [SMILES: CC(C)C[C@@H]1NC(=O)[C@@H](Cc2ccccc2)NC(=O)[C@H](CCNC(=O)OC(C)(C)C)NC(=O)[C@@H](NC(=O)N(CCNC(=O)OC(C)(C)C)NC(=O)[C@@H](NC(=O)[C@H](CCNC(=O)OC(C)(C)C)NC(=O)OCc2ccccc2)[C@@H](C)O)CCNC(=O)[C@H]([C@@H](C)O)NC(=O)[C@H](CCNC(=O)OC(C)(C)C)NC(=O)[C@H](CCNC(=O)OC(C)(C)C)NC1=O]

To a mixture of αZ-γ-boc-Dab-OH (109.2 mg, 0.31 mmol) and HATU (117.9 mg, 0.31 mmol) in anhydrous DMF (0.5 mL) was added DIEA (78 mg, 0.6 mmol). After 5 minutes, the step-c product (278.5 mg, 0.204 mmol) was added, and the reaction mixture was stirred for 30 minutes. It was directly purified by RPLC (100 g SiO2, 50% to 95% MeOH and water). Yield 303.6 mg, 87.6%. Ions found by LCMS: [(M−2Boc+2H)/2]⁺=750.0, [(M−2Boc−tBu+2H)/2]⁺=722, [(M−4Boc+2H)/2]⁺=649.8.

### Step e

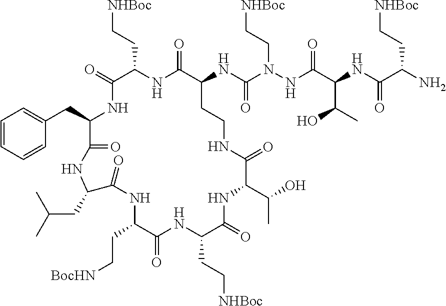 [SMILES: CC(C)C[C@@H]1NC(=O)[C@@H](Cc2ccccc2)NC(=O)[C@H](CCNC(=O)OC(C)(C)C)NC(=O)[C@@H](NC(=O)N(CCNC(=O)OC(C)(C)C)NC(=O)[C@@H](NC(=O)[C@@H](N)CCNC(=O)OC(C)(C)C)[C@@H](C)O)CCNC(=O)[C@H]([C@@H](C)O)NC(=O)[C@H](CCNC(=O)OC(C)(C)C)NC(=O)[C@H](CCNC(=O)OC(C)(C)C)NC1=O]

To the step-d product (303.6 mg, 0.179 mmol) in MeOH (10 mL) was added solution of Pd/C (dry basis, 100 mg) in water (0.3 mL):MeOH (1 mL) and 0.05 mL of 4N HCl solution in dioxane. The mixture was stirred under hydrogen atmosphere for 1 hour. Pd/C was filtered, and the filtrate was concentrated at room temperature and further dried under high vacuum. Yield 285.2 mg, quantitative yield. The product was carried to the subsequent step without further purification. Ions found by LCMS: [(M−Boc+2H)/2]⁺=733, [(M−Boc-tBu+2H)/2]⁺=705.0, [(M−2Boc+2H)/2]⁺=683, [(M−3Boc+2H)/2]⁺=633.

### Step f

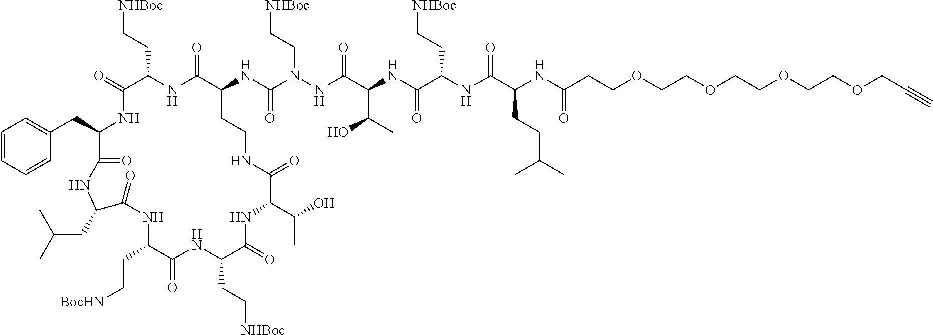 [SMILES: C#CCOCCOCCOCCOCCC(=O)N[C@@H](CCC(C)C)C(=O)N[C@@H](CCNC(=O)OC(C)(C)C)C(=O)N[C@H](C(=O)NN(CCNC(=O)OC(C)(C)C)C(=O)N[C@H]1CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](Cc2ccccc2)NC(=O)[C@H](CCNC(=O)OC(C)(C)C)NC1=O)[C@@H](C)O]

To a mixture of propargyl-PEG4-HomoLeu-OH (85.3 mg, 0.22 mmol, described in Example 226 Int-116) and HATU (92 mg, 0.242 mmol) in anhydrous DMF (0.5 mL) was added DIEPA (52 mg, 0.4 mmol). After 5 minutes, the step-e product (285.2 mg, 0.179 mmol) was added, and the reaction mixture was stirred for 30 minutes. It was then directly purified by RPLC (100 g SiO2, 50% to 90% MeOH and water, using 0.1% TFA as modifier). Yield 322.1 mg, 93%. Ions found by LCMS: [(M−2Boc+2H)/2]⁺=867.6, [(M−2Boc−tBu+2H)/2]⁺=839.5.

### Step g

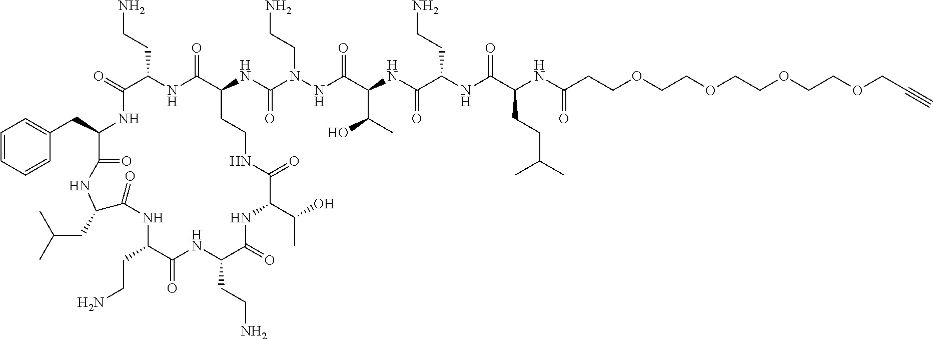 [SMILES: C#CCOCCOCCOCCOCCC(=O)N[C@@H](CCC(C)C)C(=O)N[C@@H](CCN)C(=O)N[C@H](C(=O)NN(CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O]

The step-f product (322.1 mg, 0.167 mmol) was dissolved in TFA:DCM (1:1, ˜1 mL). The solution was stirred for 20 minutes, then directly purified by HPLC (5% to 30% acetonitrile and water, using 0.1% TFA as modifier). Yield 134.2 mg, 40%. Ions found by LCMS: [(M+2H)/2]⁺=717.4, [(M+3H)/3]⁺=478.6, [(M+4H)/4]⁺=359.4.

### Example 238—Synthesis of Int-128 (Z-((S)-hexyl)-Gly]-(γ-Boc)-Dab-OH)

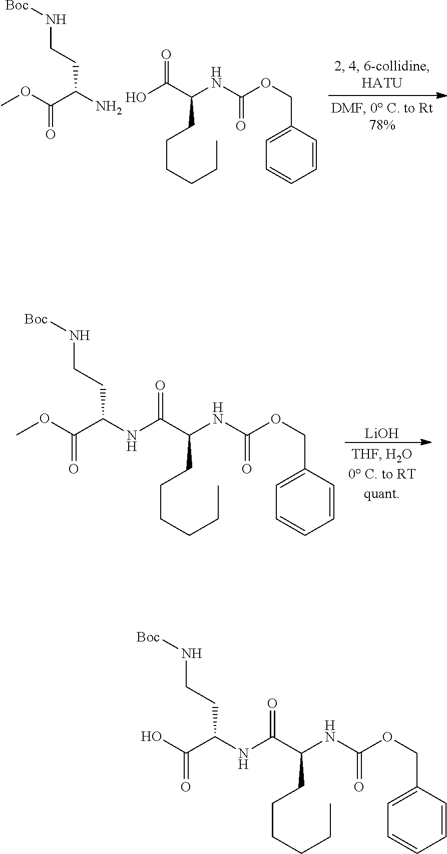

### Step a. Synthesis of [Z-((S)-hexyl)-Gly]-(γ-Boc)-Dab-OMe

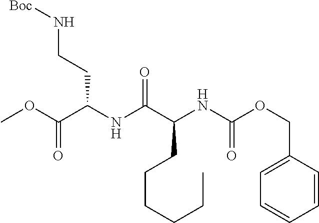 [SMILES: CCCCCC[C@H](NC(=O)OCc1ccccc1)C(=O)N[C@@H](CCNC(=O)OC(C)(C)C)C(=O)OC]

To a 0° C. stirring solution of Nγ-Boc-L-2,4-diaminobutyric acid methyl ester hydrochloride (4.219 g, 15.70 mmol), (S)-(N-benzyloxycarboxyl)-2-aminocaprilyc acid (4.606 g, 15.70 mmol), and 2,4,6-collidine (4.357 mL, 32.97 mmol) in DMF (50 mL), it was added HATU (32 mg, 0.085 mmol). Upon reaction completion, the reaction was concentrated and the product was isolated by normal phase liquid chromatography (silica gel) using an Isco CombiFlash liquid chromatograph eluted with 0% to 10% dichloromethane and methanol. Yield 6.257 g, 78% yield. 1H NMR (Methanol-d4) δ: 7.44-7.20 (m, 5H), 5.09 (br s, 2H), 4.47 (br dd, J=9.5, 4.8 Hz, 1H), 4.10 (dd, J=8.5, 5.7 Hz, 1H), 3.71 (s, 3H), 3.27-2.90 (m, 2H), 2.13-1.49 (m, 4H), 1.37 (d, J=36.9 Hz, 17H), 0.98-0.90 (t, J=7.1 Hz, 3H).

### Step b. Synthesis of [Z-((S)-hexyl)-Gly]-(γ-Boc)-Dab-OH

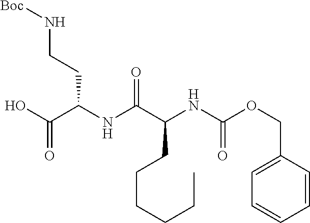 [SMILES: CCCCCC[C@H](NC(=O)OCc1ccccc1)C(=O)N[C@@H](CCNC(=O)OC(C)(C)C)C(=O)O]

To a 0° C. stirring solution of [Z-((S)-hexyl)-Gly]-(γ-Boc)-Dab-OMe (3.00 g, 5.91 mmol), in THF (48 mL), and water (12 mL) it was added lithium hydroxide (0.212 g, 8.86 mmol). The reaction was monitored by HPLC and upon completion, the temperature was raised to ambient. An excess amount of sulfonic acid resin Amberlite IRN-77 was added to the reaction and stirring was continued for 10 minutes, and pH was found acidic. The mixture was filtered through a short celite pad with the aid of methanol. The filtrate was concentrated to dryness per vacuum techniques. Yield 2.92 g. Quantitative yield. This material was used in the next step without further purification.

### Example 239—Synthesis of Int-129

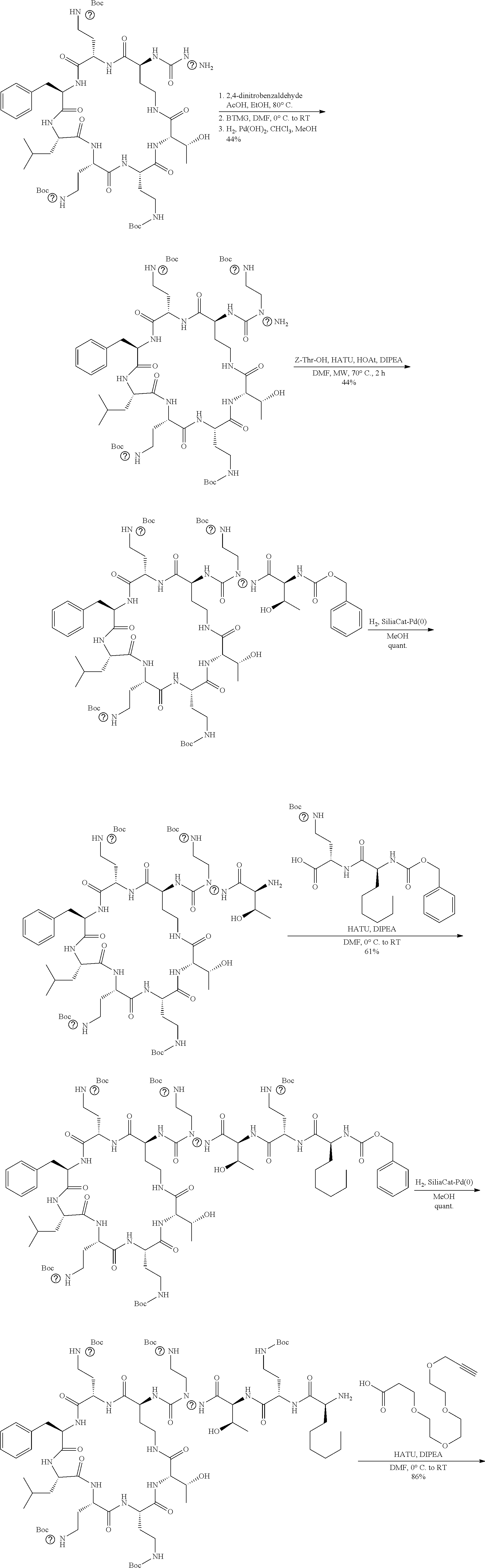

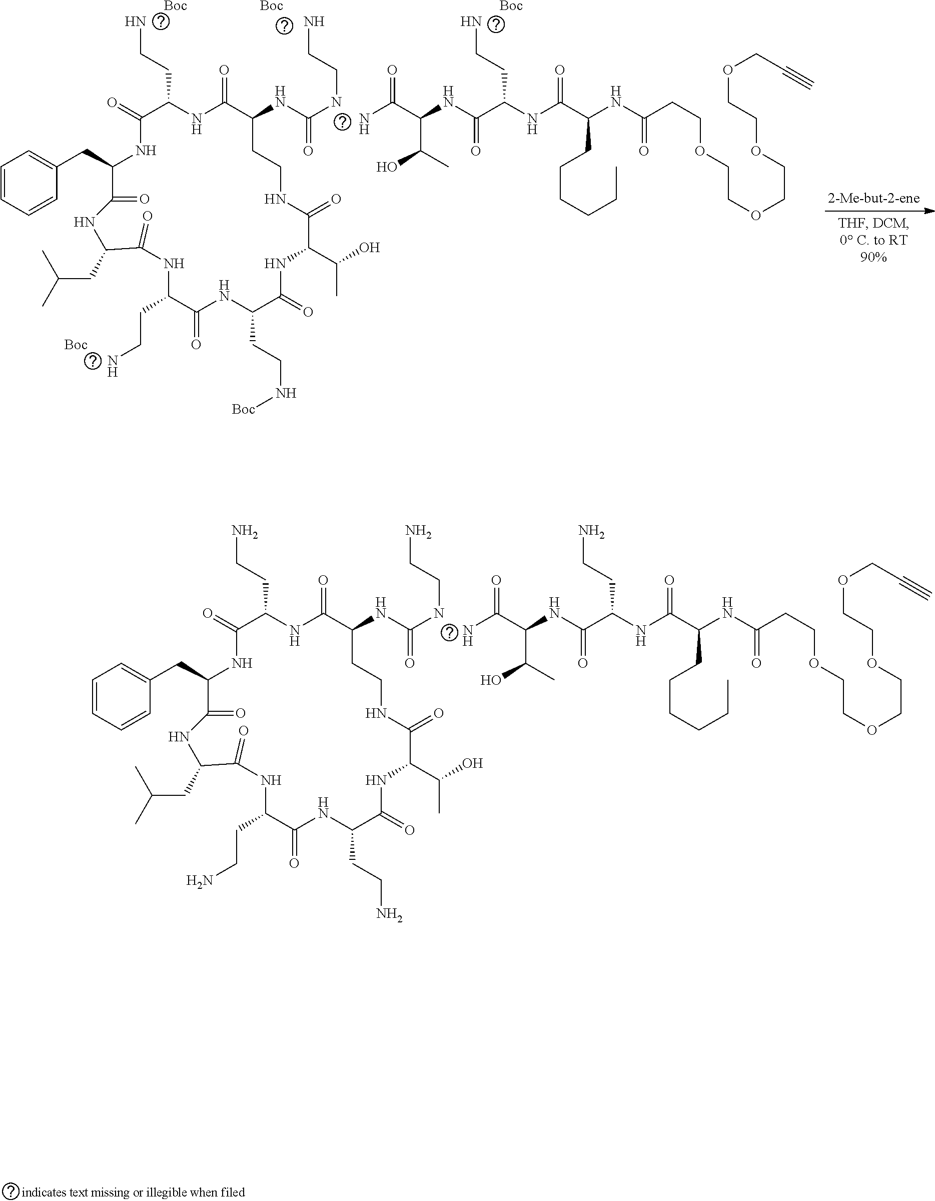

### Step a. Synthesis of Tri-Boc-PMBH-[(γ-Boc)-Aza-Dab]

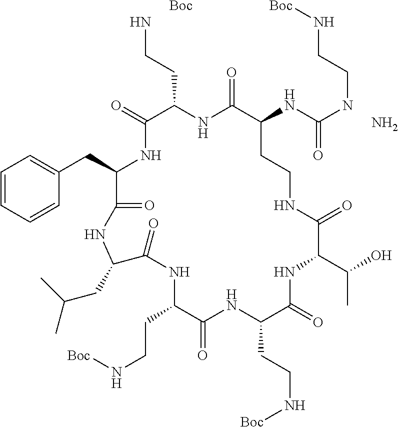

A stirring solution of Tri-Boc-PMBH-aza-Gly (10.0 g, 8.92 mmol. Step b-product, Example 192), 2,4-dinitrobenzaldehyde (1.786 g, 9.10 mmol) and acetic acid (2.5 mL) in ethanol (100 mL) was refluxed overnight. All the volatiles were evaporated per vacuum techniques. The obtained solids were suspended in water (100 mL) and stirred for 15 minutes. The mixture was filtered and the solids washed with two aliquots of water (2×100 mL). The solids were collected and all the volatiles evaporated per vacuum techniques. Yield 11.69 g, quantitative yield, and this material was used in the next step without further purification. Main ions found by LCMS: [(M−2Boc)+2H]/2=549.9, [(M−3Boc)+3H+2Na]/2=521.6, [(M−3Boc)+3H]/2=499.6. To a 0° C. stirring solution of this semicarbazone intermediate (4.50 g, 3.46 mmol) in DMF (90 mL) it was added tert-butyl 2,2-dioxo-1,2,3-oxathiazolidine-3-carboxylate (1.70 g, 7.62 mmol) and immediately after 2-tert-butyl-1,1,3,3-tetramethylguanidine (1.449 mL, 7.10 mmol). Temperature was raise to ambient and the reaction stirred overnight. HPLC analysis revealed full consumption of the starting material, and the reaction was concentrated at the rotatory evaporator. The thick crude liquor was taken up in ethyl acetate (100 mL) and treated under vigorous stirring with a 1.0 M sulfuric acid aqueous solution (100 mL). The organics were separated, dried with brine and magnesium sulfate, and then filtered. The filtrate was concentrated and all the volatiles were evaporated per vacuum techniques. Yield 5.05 g, quantitative yield, and this material was used in the next step without further purification. Main ions found by LCMS: [(M−2Boc)+2H]/2=621.4, [(M−3Boc)+3H+2Na]/2=593.4, [(M−3Boc)+3H]/2=571.1, [(M−4Boc)+4H]/2=521.5. Under hydrogen (1 atm), a suspension of N-alkylated semicarbazone (5.05 g of crude from previous step, 3.46 mmol) and 20% palladium (11) hydroxide on charcoal (2.50 g) in methanol (500 mL) and chloroform (2.5 mL), was stirred for 6 hours. The mixture was filtered over a short celite pad, and the solution was concentrated per vacuum techniques. The desired product was isolated as the TFA salt by reversed phase liquid chromatography (RPLC) using an Isco CombiFlash liquid chromatograph eluted with 30% to 100% methanol and water, using 0.1% TFA as the modifier. Yield 2.080 g, 44% yield over three steps. Ions found by LCMS: [(M−2Boc)+2H]/2=532.4: [(M−3Boc)+3H+2Na]/2=504.3, [(M−3Boc)+3H]/2=482.3, [(M−4Boc)+4H]/2=432.3.

### Step b. Synthesis of Tri-Boc-PMBH-[(γ-Boc)-Aza-Dab]-[Z-Thr]

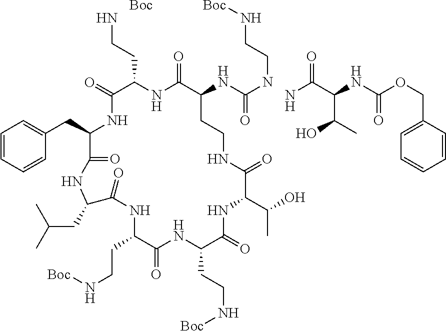

To a solution of tri-Boc-PMBH-[(γ-Boc)-aza-Dab] (250 mg, 0.181 mmol), N-carbobenzyloxy-L-threonine (138 mg, 544 mmol), 1-hydroxy-7-azabenzotriazole (132 mg, 0.580 mmol), N,N-diisopropylethylamine (0.161 mL, 0.926 mmol) in DMF (10 mL), it was added HATU (214 mg, 0.563 mmol), and the mixture was stirred and heated under microwave irradiation at 70° C. for 1 hour. Additional amounts of N-carbobenzyloxy-L-threonine (138 mg, 544 mmol), 1-hydroxy-7-azabenzotriazole (132 mg, 0.580 mmol), N,N-diisopropylethylamine (0.161 mL, 0.926 mmol) and HATU (214 mg, 0.563 mmol) were added, and the reaction was stirred and heated under microwave irradiation at 70° C. for 1 additional hour. All the volatiles were evaporated per vacuum techniques. The desired product was isolated by reversed phase liquid chromatography (RPLC) using an Isco CombiFlash liquid chromatograph eluted with 50% to 100% methanol and water, using no modifier. Yield 121 mg, 44% yield. Ions found by LCMS: [(M−2Boc)/2]+=650.0, [(M−3Boc)+3H+2Na]/2=621.8, [(M−3Boc)+3H]/2=599.7, [(M−4Boc)+4H]/2=549.8.

### Step c. Synthesis of Tri-Boc-PMBH-[(γ-Boc)-Aza-Dab]-Thr

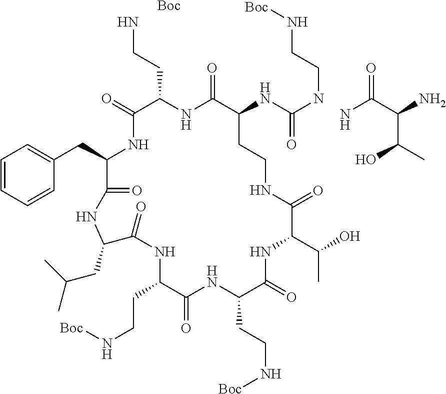

A stirring suspension of tri-Boc-PMBH-[(γ-Boc)-aza-Dab]-[Z-Thr] (121 mg, 0.081 mmol) and SiliaCat-Pd(0) (81 mg; 0.016 mmol) in methanol (5 mL) was subjected to hydrogen gas (1 atm) until full conversion was observed by HPLC analysis. The mixture was filtered through a short celite pad with the aid of methanol. The filtrate was concentrated and all the volatiles were evaporated per vacuum techniques. This material was used in the next step without further purification. Yield 111 mg, quant. Ions found by LCMS: [(M−1Boc)/1]+=632.6, [(M−2Boc)/2]+=582.8, [(M−3Boc)⁺3H+2Na]/2=604.9, [(M−3Boc)+3H]/2=533.2, [(M−3Boc)+3H+2Na]/2=555.0.

### Step d. Synthesis of Tri-Boc-PMBH-[(γ-Boc)-aza-Dab]-Thr-[(γ-Boc)-Dab]-[Z-((S)-Hexyl)-Gly]

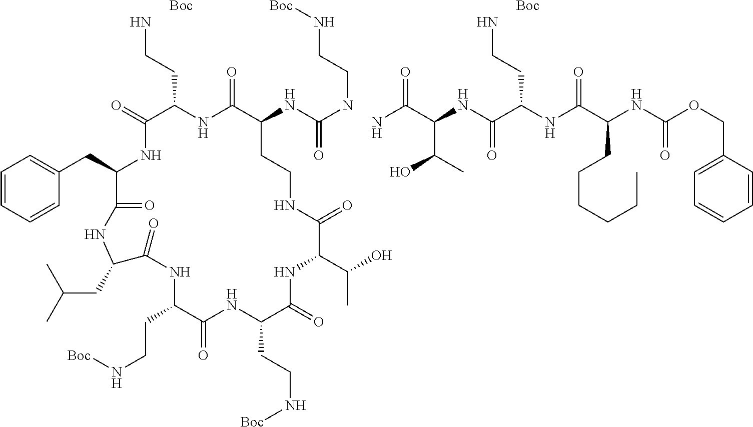

A 0° C. stirring solution of tri-Boc-PMBH-[(γ-Boc)-aza-Dab]-Thr (111 mg, 0.081 mmol), [Z-((S)-hexyl)-Gly]-(γ-Boc)-Dab-OH (Int 128) (42 mg, 0.085 mmol), and DIPEA (0.044 mL, 0.252 mmol) in DMF (3.0 mL), was treated with a solution of HATU (32 mg, 0.085 mmol) in 1.5 mL of DMF, dropwise. Upon completion, all the volatiles were evaporated per vacuum techniques. The desired product was isolated by reversed phase liquid chromatography (RPLC) using an Isco CombiFlash liquid chromatograph eluted with 50% to 100% methanol and water, using no modifier. Yield 92 mg g, 61% yield. Ions found by LCMS: [(M−2Boc)/2]+=820.5, [(M−3Boc)+3H+2Na]/2=792.5.

### Step e. Synthesis of Tri-Boc-PMBH-[(γ-Boc)-Aza-Dab]-Thr-[(γ-Boc)-Dab]-[((S)-Hexyl)-Gly]

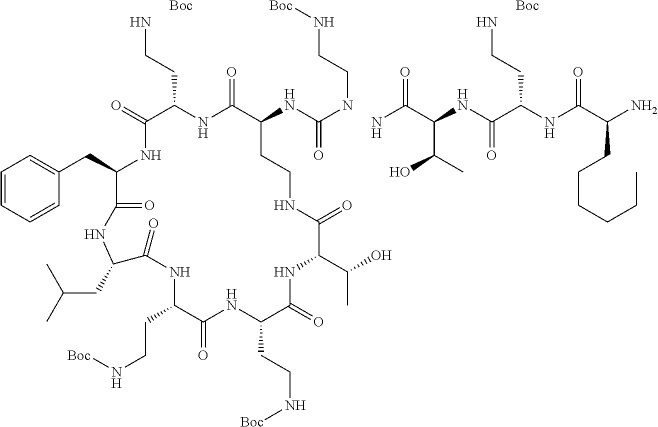

Prepared in the same fashion as tri-Boc-PMBH-[(γ-Boc)-aza-Dab]-Thr from tri-Boc-PMBH-[(γ-Boc)-aza-Dab]-Thr-[(γ-Boc)-Dab]-[Z-((S)-hexyl)-Gly] (92 mg; 0.050 mmol), SiliaCat-Pd(0) (50 mg; 0.010 mmol) and methanol (5.0 mL). Yield 85 mg, quant. Ions found by LCMS: [(M−1Boc)/1]+=803.6, [(M−2Boc)/2]+=753.5, [(M−3Boc)+3H+2Na]/2=775.7.

### Step f. Synthesis of Boc-PMBH-[(γ-Boc)-aza-Dab]-Thr-[(γ-Boc)-Dab]-[((S)-hexyl)-Gly]-[Propargyl-PEG4-Acyl]

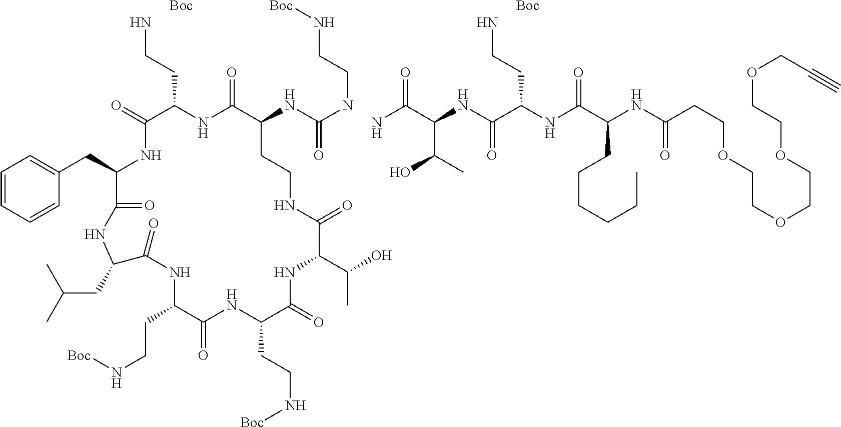

Prepared in the same fashion as tri-Boc-PMBH-[(γ-Boc)-aza-Dab]-[Z-Thr] from tri-Boc-PMBH-[(γ-Boc)-aza-Dab]-Thr-[(γ-Boc)-Dab]-[((S)-hexyl)-Gly] (85 mg, 0.050 mmol), propargyl-PEG4-acid (14 mg, 0.055 mmol), DIPEA (0.027 mL, 0.155 mmol), in DMF (3.0 mL) and HATU (20 mg; 0.052 mmol in 1.5 mL of DMF). Yield 84 mg, 86% yield. Ions found by LCMS: [(M−2Boc)/2]+=875.0, [(M−3Boc)⁺3H+2Na]/2=846.6.

### Step g. Synthesis of Int-129

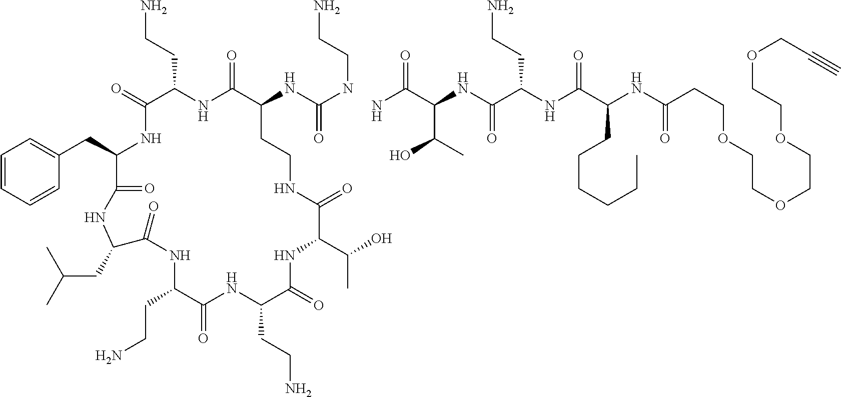

A solution of tri-Boc-PMBH-[(γ-Boc)-aza-Dab]-Thr-[(γ-Boc)-Dab]-[((S)-hexyl)-Gly]-[propargyl-PEG4-acyl] (84 mg, 0.043 mmol), dissolved in DCM (4 mL) and 2-methyl-but-2-ene (0.250 mL), was treated with TFA (2 mL), while stirring at room temperature. After 30 minutes, all the volatiles were evaporated per vacuum techniques. The desired product was isolated as the penta-TFA salt by reversed phase liquid chromatography (RPLC) using an Isco CombiFlash liquid chromatograph eluted with 0% to 100% methanol and water, using TFA as the modifier. Yield 78 mg, 90% yield. Main ions found by LCMS: (M+2H)/2=724.4, (M+3H)/3=483.4. (M+4H)/4=382.8.

### Example 240—Synthesis of Conjugate 90

A solution of azido functionalized hIgG1 Fc (SEQ ID NO: 17) (100 mg, 5.63 mL, 1.718 μmol) was added to a 15 mL centrifuge tube containing alkyne functionalized small molecule (66.3 mg, 0.03694 mmol, described in Example 225 Int-115). After gently shaking to dissolve all solids, the mixture was charged with 257.7 μL of a mixture solution of THPTA (43 mg, 0.1 mmol), L-ascorbic acid sodium (60 mg, 0.3 mmol), and copper (II) sulfate (15.9 mg, 0.1 mmol) in PBS 7.4 buffer (1 mL). The resulting mixture was gently rocked overnight. It was purified by affinity chromatography over a protein A column, followed size exclusion chromatography (see Example 214 for procedure). Maldi TOF analysis of the purified final product gave an average mass of 64777. Da (DAR=4.1). Yield 52 mg, 52% yield.

### Example 241—Synthesis of Conjugate 91

### Step a. Synthesis of PEG4-azido Fc

PEG4-azidoNHS ester (98%, 164 μmol, 9.5 equivalents, 65.2 mg in 0.40 mL of DMF and diluted to 3.29 mL with pH 7.4 PBS 1× buffer solution) was added to a solution of h-IgG1 Fc (1.007 g in 65.0 mL of pH 7.4 PBS, MW˜58,200 Da, 17.3 μmol) and the mixture was shaken gently for 12 hours at ambient temperature. The solution was concentrated using a centrifugal concentrator (30,000 MWCO) to a volume of ˜1.5 mL. The crude mixture was diluted 1:10 in PBS pH 7.4, and concentrated again. This wash procedure was repeated for total of three times. The concentrated Fc-PEG4-azide was diluted to 65.0 mL with pH 7.4 PBS 1× buffer. The purified material was quantified using a Nanodrop™ UV visible spectrophotometer (using a calculated extinction coefficient based on the amino acid sequence of h-IgG1). Yield was quantitative after purification. DAR=2.7 determined by MALDI. The DAR value can be adjusted by altering the equivalents of PEG4-azido NHS ester in near linear relationship.

### Step b. Synthesis of Conjugate 91

To a solution of h-IgG1 Fc-azide (6.452 mL, 1.72 μmol, 15.5 mg/mL, MW=58200 Da) in pH 7.4 PBS 1× were added alkyne derivatized small molecule Int-120 (244.2 mg, 37.8 μmol) and freshly prepared pH 7.4 PBS solutions of CuSO₄ (0.687 mL of 5.0 mM, 20 eq), tris(3-hydroxypropyltriazolylmethyl)-amine (THPTA, 0.687 mL of 5.0 mM, 20 eq), and sodium ascorbate (1.031 mL of 5.0 mM, 30 eq). The resulting solution was agitated gently for overnight at room temperature. The reaction mixture was purified by affinity chromatography over a protein A column, followed by size exclusion chromatography over Superdex200. MALDI TOF analysis of the purified final product gave an average mass of 66091 Da (DAR=2.7). Yield 60.7 mg, 61% yield.

### Example 242—Synthesis of Conjugate 92

A solution of azido-Peg-4-Fc in PBS buffer solution (100 mg, 1.28 μmol, 6.45 mL, MW=58200 Da, DAR=4.3) was combined with alkyne derivatized small molecule Int-121 (28 mg, 0.036 mmol) and freshly prepared pH 7.4 PBS solutions of CuSO₄ (0.68 mL of 50.0 mM, 20 eq), tris(3-hydroxypropyltriazolylmethyl)-amine (THPTA, 0.68 mL of 50.0 mM, 20 eq), and sodium ascorbate (1.00 mL of 50.0 mM, 30 eq). The resulting homogeneous solution was agitated by rocker table for 12 h. The crude solutions were diluted with pH 7.4 PBS to a final concentration of 1 mg/mL, and ultrafiltered (10.000 MWCO) to a volume of 1 mL, two times. The crude mixtures were then diluted 1:10 in PBS pH 7.4, and purified using MabSelect Sure Resin (GE Healthcare, Chicago, Ill., USA), followed by size exclusion chromatography. Purified material was quantified using a Nanodrop™ UV visible spectrophotometer (using a calculated extinction coefficient based on the amino acid sequence of the Fc used in the conjugation, and concentrated to approximately 10 mg/mL using a centrifugal concentrator (10,000 MWCO). Purified molecules were analyzed using 4-12% Bis Tris SDS PAGE gels by loading 1-2 μg of each molecule into the gel, and staining using instant Blue staining. Each gel included a molecular weight ladder with the indicated molecular weight standards. Yields are typically 40-60%. MALDI MS analysis showed a range of masses (70000-90000) with an average of mass of 66543. Average DAR=2.9.

### Example 243—Synthesis of Conjugate 93

### Step a. Synthesis of PEG4-azido Fc

PEG4-azidoNHS ester (98%, 164 μmol, 9.5 equivalents, 65.2 mg in 0.40 mL of DMF and diluted to 3.29 mL with pH 7.4 PBS 1× buffer solution) was added to a solution of h-IgG1 Fc (SEQ ID NO: 17) (1.007 g in 65.0 mL of pH 7.4 PBS, MW-58,200 Da, 17.3 μmol) and the mixture was shaken gently for 12 hours at ambient temperature. The solution was concentrated using a centrifugal concentrator (30,000 MWCO) to a volume of ˜1.5 mL. The crude mixture was diluted 1:10 in PBS pH 7.4, and concentrated again. This wash procedure was repeated for total of three times. The concentrated Fc-PEG4-azide was diluted to 65.0 mL with pH 7.4 PBS 1× buffer. The purified material was quantified using a Nanodrop™ UV visible spectrophotometer (using a calculated extinction coefficient based on the amino acid sequence of h-IgG1). Yield was quantitative after purification. DAR=2.7 determined by MALDI. The DAR value can be adjusted by altering the equivalents of PEG4-azido NHS ester in near linear relationship.

### Step b. Synthesis of Conjugate 93

To a solution of h-IgG1 Fc-azide (5.73 mL, 17.2 μmol, 17.5 mg/mL. MW=58,200 Da) in pH 7.4 PBS 1× were added alkyne derivatized small molecule (TFA salt, 125.4 mg, 34.4 μmol, described in Example 232 Int-122) and freshly prepared pH 7.4 PBS×1 solutions of CuSO₄ (0.687 mL of 5.0 mM, 20 eq), tris(3-hydroxypropyltriazolylmethyl)-amine (THPTA, 0.687 mL of 5.0 mM, 20 eq), and sodium ascorbate (1.031 mL of 5.0 mM, 30 eq). The resulting solution was rocked gently for overnight at room temperature. The reaction mixture was purified by affinity chromatography over a protein A column, followed by size exclusion chromatography with Superdex200. MALDI TOF analysis of the purified final product gave an average mass of 67051 Da (DAR=3.1). Yield 56.3 mg, 56% yield.

### Example 244—Synthesis of Conjugate 94

A solution of azido-Peg-4-Fc in PBS buffer solution (80 mg, 1.1 μmol, 4.8 mL, MW=58200 Da, DAR=4.3) was combined with material from Example 233 Int-123 (70 mg, 0.019 mmol) and freshly prepared pH 7.4 PBS solutions of CuSO₄ (0.54 mL of 50.0 mM, 20 eq), tris(3-hydroxypropyltriazolylmethyl)-amine (THPTA, 0.54 mL of 50.0 mM, 20 eq), and sodium ascorbate (0.82 mL of 50.0 mM, 30 eq). The resulting homogeneous solution was agitated by rocker table for 12 h. The crude solutions were diluted with pH 7.4 PBS to a final concentration of 1 mg/mL, and ultrafiltered (10,000 MWCO) to a volume of 1 mL, two times. The crude mixtures were then diluted 1:10 in PBS pH 7.4, and purified using MabSelect Sure Resin (GE Healthcare, Chicago, Ill., USA), followed by size exclusion chromatography. Purified material was quantified using a Nanodrop™ UV visible spectrophotometer (using a calculated extinction coefficient based on the amino acid sequence of the Fc used in the conjugation, and concentrated to approximately 10 mg/mL using a centrifugal concentrator (10,000 MWCO). Purified molecules were analyzed using 4-12% Bis Tris SDS PAGE gels by loading 1-2 μg of each molecule into the gel, and staining using instant Blue staining. Each gel included a molecular weight ladder with the indicated molecular weight standards. Maldi TOF analysis of the purified final product gave an average mass of 69435 Da (DAR=3.8). Yield 49.4 mg, 62% yield.

### Example 246—Synthesis of Conjugate 95

The title compound was prepared analogously to Example 240 Conjugate 90, using alkyne functionalized small molecule described in Example 237 Int-127. Maldi TOF analysis of the purified final product gave an average mass of 62085 Da (DAR=2.3). Yield 59 mg, 49% yield.

### Example 246—Synthesis of Conjugate 96

Alkyne derivatized small molecule (55 mg, 0.027 mmol, described in Example 239 Int-129) was added to azido h-IgG1 (c-myc) Fc (SEQ ID NO: 17) (0.0003 M, 4.296 mL, DAR 4) solution, followed by 0.966 mL of 0.02 M CuSO₄ solution, 2.900 mL of 0.02 M sodium ascorbate solution, and 0.966 mL of 0.02 M THPTA solution. The reaction was gently rocked at room temperature overnight. The crude mixture was diluted 1:10 in PBS pH 7.4, and purified using MabSelect Sure Resin (GE Healthcare, Chicago, Ill., USA), followed by size exclusion chromatography. Purified material was quantified using a Nanodrop™ UV visible spectrophotometer (using a calculated extinction coefficient based on the amino acid sequence of h-IgG1), and concentrated to approximately 10 mg/mL using a centrifugal concentrator (10,000 MWCO). Purified molecules were analyzed using 4-12% Bis Tris SDS PAGE gels by loading 1-2 μg of each molecule into the gel, and staining using instant Blue staining. Each gel included a molecular weight ladder with the indicated molecular weight standards (FIG. 29). “NR” indicates non-reduced SDS-PAGE gel condition. Maldi TOF analysis of the purified final product gave an average mass of 62602. Da (DAR=2.5). Yield 27.1 mg, 36% yield.

### Example 247—Synthesis of Conjugate 97

### Step a. Synthesis of BCN Modified Fc

BCN-PEG4-NHS ester (16 mg, 0.029 mmol, MW=538.6, BCN=bicyclononyne), in 0.062 mL of DMF was added to a solution of h-IgG1 (c-myc) Fc (SEQ ID NO: 17) (168 mg, 0.0029 mmol) in PBS 7.4 Buffer (10 mL) and the mixture was rocked overnight and then filtered (10×dilution) through a centricon tube (MW cuttoff >30K) 3 times. The resulting solution was gently rocked overnight. It was purified by affinity chromatography over a protein A column, followed size exclusion chromatography. Maldi TOF analysis of the purified final product gave an average mass of 59200. Yield 85 mg, 49% yield.

### Step b. Copper Free Click Conjugation

24.8 mg of Int-7 (4.9 mg, 0.0013 mmol) was added to BCN-functionalized Fc from above (25 mg, 0.00042 mmol) in 1.5 mL of 1× PBS 7.4 buffer and the mixture was gently rocked for 18 hours. The crude solutions were diluted with pH 7.4 PBS to a final concentration of 1 mg/mL, and ultrafiltered (10,000 MWCO) to a volume of 1 mL, two times. The crude mixtures were then diluted 1:10 in PBS pH 7.4, and purified using MabSelect Sure Resin (GE Healthcare, Chicago, Ill., USA), followed by size exclusion chromatography. Purified material was quantified using a Nanodrop™ UV visible spectrophotometer (using a calculated extinction coefficient based on the amino acid sequence of the Fc used in the conjugation, and concentrated to approximately 10 mg/mL using a centrifugal concentrator (10,000 MWCO). Purified molecules were analyzed using 4-12% Bis Tris SDS PAGE gels by loading 1-2 μg of each molecule into the gel, and staining using instant blue staining. Each gel included a molecular weight ladder with the indicated molecular weight standards. Yields are typically 40-60%. MALDI MS analysis showed a range of masses (70000-90000) with an average of mass of 61720. Average DAR=1.1, Yield 17 mg, 65% yield.

### Example 248—Preparation of Conjugate 98

A solution of copper (II) sulfate (10 mM, 15.96 mg, 0.01 mmol), THPTA (10 mM, 43.86 mg, 0.01 mmol, THPTA=tris(3-hydroxypropyltriazolylmethyl)amine), and sodium ascorbate (15 mM, 39.6 mg, 0.20 mmol) was prepared in 10 mL of pH 7.4 PBS. To a solution of alkyne derivatized small molecule Int-116 TFA salt (81.56 mg, 0.043 mmol) and azido functionalized hIgG1 Fc (100.3 mg, azido DAR=2.9, concentration=15.43 mg/mL pH 7.4 PBS) was added the prepared solution of copper (II), THPTA and sodium ascorbate (6.5 mL). The resulting homogeneous solution was agitated by rocker table for 12 h, then purified by protein A affinity chromatography, followed by size exclusion chromatography over Superdex 200. Yield of product was 64%. MALDI MS analysis showed an average mass of 64178. Average DAR=3.5.

### Example 249—Minimum Inhibitory Concentration (MIC) Assays

MIC assays were performed according to CLSI broth microdilution guidelines (M07-A9, M100-S23) with the exception of using a 100 μL assay volume, using RPMI media instead of Mueller-Hinton media for the drug conjugates and preparing stock compounds at 12.5× final concentration. Briefly, stock solutions of all antibacterial agents were prepared fresh in appropriate solvents (i.e. PBS, pH 7.4, DMSO, DI, etc.). Stock concentrations for drug conjugates were made at 12.5× the highest final assay concentration. All were serially diluted 2-fold, 8 or 12 times in a 96-well PCR plate (VWR cat. no. 83007-374). Bacterial cell suspensions generated from Mueller-Hinton agar (MHA) plate cultures were prepared in 0.85% saline and adjusted to ˜0.1 OD₆₀₀ nm. Next, cell suspensions were diluted 1:200 in RPMI containing 5% Luria Broth (LB) to improve bacterial growth (BD Difco, cat. no. 244620) to a concentration of ˜5×10⁵ CFU (colony-forming units)/mL. Due to poor solubility in Mueller-Hinton broth media, drug conjugates were evaluated in RPMI 1640 medium (+) L-glutamine, (+) sodium bicarbonate, pH 7.4 (“RPMI B”; Gibco, cat. no. 11875-085) as has been described as a more physiologically-relevant media for susceptibility testing of antibacterial compounds (1). For the conjugates, 92 μL of each cell suspension in RPMI B were added in the same 96-well plate assay format. A Rainin model 20 μL Liquidator 96 was used to dispense 8 μL of each 12.5× stock compound into the plate containing 92 μL of each strain in RPMI B. For the conjugates, 8 μL volumes were dispensed to achieve higher MIC testing concentrations due to the limits of conjugate stock concentrations. Compounds were tested against a 14-strain panel comprised of E. coli (Ec), A. baumannii (Ab), P. aeruginosa (Pa), and K. pneumoniae (Kp) that were obtained from ATCC (Ec 25922, Ec 2489, Ab 19606, Pa 27853, Pa PAO1, Kp 43816, Kp 10031), Centers for Disease Control and Prevention (Ec AR0349), BEI Resources (Ab AB5075), Micromyx LLC (Ab 8990, Kp 6951), and Belgian Coordinated Collections of Microorganisms (Pa LES431) or generated in-house (Ec 25922 64X-1). Four of the strains were colistin-resistant examples (COL^{R}) including: Ec 64X-1 (spontaneous mutant possessing a mutation in pmrB (A159V) that was selected by plating on MHA containing colistin at 64-fold the MIC concentration). Ec AR0349 (clinical isolate possessing the mobile mcr-1 colistin resistance gene), Ab 8990 (a clinical isolate possessing unknown colistin resistance mechanisms) and Kp 6951 (clinical isolate possessing a mutation in phoQ (T244N)). E. coli strain ATCC 25922 and P. aeruginosa strain LES431 were run in the presence and absence of 50% heat-inactivated fetal bovine serum (FBS; Sigma, cat. no. F4135-100 mL). Plates were mixed by shaking then incubated at 35° C. overnight (16-20 h). MIC values were read visually at 100% growth inhibition for conjugates human IgG1 Fc (Table 10).

TABLE 10

| | | MIC (μg/ml) | | | | | | | | | | | | | | | | |
|---|---|---|---|---|---|---|---|---|---|---|---|---|---|---|---|---|---|---|
| | | | | | | | | | Ab | | | | | | | | | |
| | | | | EC | Ec | | | | AB58705 | | | | | | | | | |
| | | | Ec | 25922 | AR0349 | | | | (COL^{R}) | Ab | | | | Pa | | | Kp | |
| | Conjugate or | Ec | 25922 + | 64X-1 | (mcr-1, | Ec | Ab 19606 | Ab | IpxD, mut. | 8990 | Pa | Pa | Pa | LES431 + | Kp | Kp | 6951 | |
| Media | Intermediate | 25922 | FBS | (COL^{R}) | COL^{R}) | 2469 | (COLhet^{R}) | AB5075 | (no LPS) | (COL^{R}) | 27853 | PAO1 | LES431 | FBS | 10031 | 43816 | (COL^{R}) | Sa RN |
| CA-MHB | Int-17 | 2 | ND | 64 | 16 | 1 | 1 | 1 | ND | ND | 2 | 4 | 1 | ND | 1 | 1 | >128 | ND |
| CA-MHB | Int-41 | 4 | 0.5 | 4 | 4 | 2 | 4 | 4 | ND | 128 | 2 | 2 | 2 | 2 | 4 | 4 | 16 | >128 |
| CA-MHB | Int-105 | 2 | 0.5 | 4 | 4 | 2 | 2 | 2 | ND | >128 | 4 | 4 | 2 | 16 | 2 | 2 | 32 | ND |
| CA-MHB | Int-107 | 2 | 0.5 | 4 | 2 | 2 | 2 | 2 | ND | >128 | 2 | 2 | 2 | 8 | 2 | 2 | 16 | ND |
| CA-MHB | Int-111 | 4 | 8 | 4 | 4 | 4 | 4 | 4 | ND | 32 | 4 | 8 | 4 | 32 | 4 | 4 | 8 | ND |
| CA-MHB | Int-115 | 2 | 2 | 64 | 16 | 2 | 4 | 2 | 64 | >128 | 4 | 8 | 4 | 16 | 2 | 2 | 128 | >128 |
| CA-MHB | Int-116 | 2 | 2 | 64 | 8 | 2 | 1 | 1 | 64 | >128 | 4 | 4 | 4 | 8 | 2 | 2 | >128 | >128 |
| CA-MHB | Int-117 | 8 | 16 | 8 | 8 | 8 | 8 | 8 | 8 | 8 | 8 | 16 | 4 | 64 | 8 | 8 | 16 | 16 |
| CA-MHB | Int-118 | 1 | 0.5 | 32 | 4 | 1 | 2 | 1 | 16 | 128 | 2 | 2 | 1 | 2 | 2 | 2 | >128 | >128 |
| CA-MHB | Int-119 | 2 | 2 | 64 | 16 | 2 | 2 | 1 | 64 | >128 | 4 | 16 | 4 | 8 | 2 | 2 | >128 | >128 |
| CA-MHB | Int-120 | 2 | 0.25 | 2 | 2 | 2 | 2 | 2 | 4 | >128 | 4 | 4 | 2 | 8 | 2 | 2 | 8 | >128 |
| CA-MHB | Int-121 | 2 | 0.5 | 16 | 4 | 2 | 2 | 2 | 4 | >128 | 4 | 8 | 2 | 8 | 2 | 2 | 32 | >128 |
| CA-MHB | Int-122 | 2 | 0.25 | 2 | 2 | 2 | 2 | 2 | 2 | >128 | 4 | 4 | 2 | 8 | 2 | 2 | 8 | >128 |
| CA-MHB | Int-123 | 4 | 1 | 4 | 4 | 2 | 4 | 4 | 4 | >128 | 4 | 4 | 2 | 8 | 4 | 4 | 4 | >128 |
| CA-MHB | Int-124 | 4 | 2 | 4 | 8 | 4 | 4 | 4 | 4 | 32 | 4 | 4 | 4 | 4 | 4 | 4 | 32 | 128 |
| CA-MHB | Int-125 | 1 | 0.5 | 64 | 16 | 1 | 4 | 2 | 32 | >128 | 2 | 2 | 1 | 4 | 1 | 1 | >128 | >128 |
| CA-MHB | Int-126 | 1 | 0.25 | 64 | 8 | 0.5 | 4 | 2 | 64 | >128 | 2 | 2 | 4 | 4 | 1 | 1 | >128 | >128 |
| CA-MHB | Int-127 | 1 | 0.5 | 64 | 8 | 1 | 2 | 2 | 64 | >128 | 2 | 2 | 1 | 4 | 1 | 1 | >128 | >128 |
| CA-MHB | Int-129 | 1 | 0.5 | 64 | 8 | 2 | 2 | 2 | 32 | 128 | 4 | 8 | 2 | 16 | 2 | 2 | >128 | >128 |
| RPMI B | Conjugate 80 | 2 | 0.5 | 16 | 4 | 2 | 2 | 2 | >128 | 4 | 8 | 2 | 8 | 2 | 2 | 32 | >256 | >256 |
| RPMI B | Conjugate 81 | 64 | 256 | 64 | 128 | 64 | 32 | 16 | ND | >256 | >256 | >256 | 32 | 256 | 256 | 256 | 256 | >256 |
| RPMI B | Conjugate 82 | 128 | 256 | 64 | 256 | 128 | 64 | 32 | ND | >256 | >256 | >256 | 64 | 256 | 256 | >256 | >256 | >256 |
| RPMI B | Conjugate 83 | 256 | >256 | 256 | >256 | 256 | >256 | 256 | ND | >256 | >256 | >256 | 128 | >256 | 256 | >256 | >256 | >256 |
| RPMI B | Conjugate 85 | >256 | >256 | >256 | >256 | >256 | 256 | 128 | ND | >256 | >256 | >256 | >256 | >256 | >256 | >256 | >256 | >256 |
| RPMI B | Conjugate 86 | 256 | 256 | 128 | 256 | 128 | 256 | 64 | ND | >256 | >256 | >256 | 128 | >256 | 256 | >256 | >256 | >256 |
| RPMI B | Conjugate 87 | >256 | >256 | >256 | >256 | >256 | >256 | 256 | ND | >256 | >256 | >256 | >256 | >256 | >256 | >256 | >256 | >256 |
| RPMI B | Conjugate 88 | 256 | 256 | 128 | >256 | 256 | 32 | 32 | >256 | >256 | >256 | >256 | 128 | >256 | >256 | >256 | >256 | >256 |
| RPMI B | Conjugate 89 | 64 | 256 | 64 | 128 | 256 | 32 | 32 | >256 | >256 | >256 | >256 | 128 | >256 | >256 | >256 | 256 | >256 |
| RPMI B | Conjugate 90 | >512 | >512 | 512 | >512 | >512 | >512 | 256 | >512 | >512 | >512 | >512 | >512 | >512 | >512 | >512 | >512 | >512 |
| RPMI B | Conjugate 91 | 64 | 128 | 64 | 128 | 64 | 16 | 16 | >512 | >512 | >512 | >512 | 512 | >512 | 512 | 512 | 256 | >512 |
| RPMI B | Conjugate 92 | 64 | 128 | 64 | 128 | 64 | 32 | 32 | >512 | >512 | >512 | >512 | 512 | >512 | 256 | 512 | 512 | >512 |
| RPMI B | Conjugate 93 | 64 | 64 | 64 | 128 | 64 | 32 | 32 | >512 | >512 | >512 | >512 | 256 | >512 | 512 | 512 | 256 | >512 |
| RPMI B | Conjugate 94 | 256 | 256 | 128 | 256 | 128 | 32 | 32 | >512 | >512 | >512 | >512 | 256 | >512 | >512 | >512 | 512 | >512 |
| RPMI B | Conjugate 95 | >256 | >256 | 256 | >256 | >256 | 256 | 256 | >256 | >256 | >256 | >256 | >256 | >256 | >256 | >256 | >256 | >256 |
| RPMI B | Conjugate 96 | >512 | >512 | >512 | >512 | >512 | 512 | >512 | >512 | >512 | >512 | >512 | >512 | >512 | >512 | >512 | >512 | >512 |
| RPMI B | Conjugate 97 | 256 | 512 | 128 | 256 | 256 | 128 | 128 | >512 | >512 | >512 | >512 | 256 | >256 | >512 | >512 | >512 | >512 |
| RPMI B | Conjugate 98 | >512 | >512 | ND | >512 | ND | ND | 256 | ND | ND | ND | >512 | >512 | >512 | ND | >512 | ND | ND |

Abbreviations:
Ec-Escherichia coli,
Ab-Acinetobacter baumannii,
Pa-Pseudomonas aeruginosa,
Kp-Klebsiella pneumoniae,
COL^{R}-colistin-resistant,
COLhet^{R}-colistin-heteroresistant,
FBS-fetal bovine serum (50%, heat-inactivated),
ND-not determined

### Example 250—Protective Effect of Fc Conjugates 82, 83, 85, 86, and 87 Against Endotoxemia

Septic shock, resulting in systemic organ failure, is an important component of many bacterial infections. In infections caused by Gm-negative pathogens, or in some mixed infections, LPS (aka endotoxin) is the dominate mediator of this process which results from hyper-stimulation of the immune system. In a mouse model of endotoxemia we were able to demonstrate the protective effect of Fc conjugates to rescue mice given a lethal dose of LPS.

Sensitized mouse model of endotoxemia. These studies used female immune competent C57BL/6 mice (Jackson Laboratories) between 8 and 10 weeks old. After acclimation for 7 days, mice were challenged IP with 18 ng of purified LPS (Escherichia coli 0111:B4; Sigma #L4130) in PBS mixed with 20 mg of D-galactosamine hydrochloride (Fisher #50-492-412) in a volume of 0.4 ml. Previously this dose was shown to result in 100% mortality of challenged mice within 5 days. The murine LD₁₀₀ of this model is approximately equal to that of humans due to sensitization by D-galactosamine. Study mice were housed and cared for following standard IACUC approved protocols, and clearly moribund mice were humanely euthanized and scored as a death.

Efficacy of Fc conjugates in endotoxemia. C57BL/6 mice were challenged with a lethal dose of LPS as described above. One hour prior to LPS challenge control groups of animals received vehicle (PBS), colistin at 5 mg/kg, or Fc only (hIgG1) at the concentration indicated in Table 11 by IV injection. Fc conjugates were also administered IV at T-1 hour (5 and 25 mg/kg in study 1, and 1, 3, 10 and 30 mg/kg in study 2). As illustrated in Table 11, the majority of mice dosed with vehicle or hIgG1 (Fc only) reached mortality within 48 hours of LPS challenge. In contrast, mice treated with 5 mg/kg colistin achieved 80-100% survival across both studies while Fc conjugates reached 100% survival at the concentrations listed in Table 11. These studies demonstrate the LPS neutralizing capabilities of Fc conjugates and suggest an additional protective mechanism beyond direct killing of bacteria.

TABLE 11
Protection against lethal LPS challenge by Fc conjugates in a mouse
endotoxemia model. Survival at 48 hours post-LPS challenge.
Fc Conjugates

| Conjugate Number | Dose (mg/kg) | % Survival |
|---|---|---|
| Study 1: | | |
| hlgG1 | 25 | 20 |
| Conjugate 82 | 25 | 100 |
| Conjugate 83 | 25 | 100 |
| Conjugate 86 | 5 | 100 |
| Study 2: | | |
| hlgG1 | 30 | 0-20 |
| Conjugate 85 | 30 | 100 |
| Conjugate 87 | 10 | 100 |

### Example 251—Protective Effect of Fc Conjugates 23 and 22 Against Multiple LPS Types in Mouse Lethal Endotoxemia

Sensitized mouse model of endotoxemia. These studies were conducted as described elsewhere except for the type of LPS used. In order to determine the ability of our Fc conjugates to neutralize a broad range of LPS types, the endotoxin from multiple important species were employed as the lethal agent in endotoxemia studies. LIPS types included E. coil 0111:B34 (Sigma L4130), Proteus mirabilis (Sigma SMB00704), Klebsiella pneumoniae (Sigma L4268), Pseudomonas aeruginosa 10 (Sigma L7018), and Escherichia coli 026:1B6 (Sigma L3755). In all studies an LD₉₅₋₁₀₀ was used and the end point was 48 hours, except for Proteus in which the end point was 24 hours.

Ability of Fc conjugates to neutralize multiple LPS types. C57BL/6 mice were challenged with a lethal dose of LPS as described above and elsewhere. One hour prior to LPS challenge control groups of animals received vehicle (PBS), or Fc only (hIgG1) at 25 mg/kg by IV injection. Fc conjugates (either conjugate 23 or conjugate 22) were administered IV at T-1 hour at a 25 mg/kg dose. As indicated in Table 12, only 0-20% of mice dosed with vehicle or hIgG1 survived for 24-48 hours post LIPS challenge, regardless of the LPS type. In contrast, mice treated with Fc conjugates at 25 mg/kg achieved 80-100% survival. These studies demonstrate the ability of Fc conjugates to neutralize the LPS from multiple clinically relevant Gm-negative pathogens.

TABLE 12
LPS Range

| LPS Source | Dose (mg/kg) | % Survival |
|---|---|---|
| E. coli 0111:B4 | | |
| PBS | N/A | 0 |
| hlgG1 | 25 | 0 |
| Conjugate 22 | 25 | 100 |
| E. coli 026:B6 | | |
| PBS | N/A | 0 |
| hlgG1 | 25 | 0 |
| Conjugate 22 | 25 | 100 |
| K. pneumoniae | | |
| PBS | N/A | 20 |
| hlgG1 | 25 | 0 |
| Conjugate 23 | 25 | 100 |
| Proteus mirabilis* | | |
| PBS | N/A | 20 |
| hlgG1 | 25 | 0 |
| Conjugate 22 | 25 | 80 |
| Serratia | | |
| PBS | N/A | 0 |
| hlgG1 | 25 | 0 |
| Conjugate 23 | 25 | 80 |
| Pseudomonas 10 | | |
| PBS | N/A | 20 |
| hlgG1 | 25 | 0 |
| Conjugate 22 | 25 | 100 |

* = 24 hour time point.

### Example 252—Prophylactic Dosing of Fc Conjugates 27 and 87 in a Lethal Mouse Endotoxemia Model

Sensitized mouse model of endotoxemia. This study was conducted using the previously described D-galactosamine endotoxemia model. The only alteration was the timing of the dose, with Fc conjugates administered between 2 and 48 hours prior to LPS challenge (IV). This was to assess the utility of these conjugates as prophylactic therapeutics in patients with increased risk of developing septic infections.

Efficacy of Fc conjugates in endotoxemia. In this study two conjugates were evaluated, conjugate 27 is an un-stabilized molecule while conjugate 87 has been specifically modified to increase its resistance to proteolysis. Both molecules were dosed in separate groups of mice (n=5) at 2, 24, or 48 hours prior to challenge with a lethal dose of LPS. The un-stabilized construct (conjugate 27) demonstrated complete protection when administered at T-2 and partial protection at T-24, however it failed to afford significant protection when given 48 hours prior to LPS challenge. In contrast, conjugate 87 shows complete protection at both T-2 and T-24 and partial protection at 48 hours prior to LPS administration. This data suggests that stabilized Fc constructs have therapeutic potential as prophylactic agents in vulnerable patient populations.

TABLE 13
Prophylactic evaluation of Fc conjugates in a mouse
endotoxemia model. Survival at 48 hours post-LPS challenge.
Prophylaxis

| Conjugate Number | Dose Timing (hour) | % Survival |
|---|---|---|
| Vehicle (PBS) | −2 | 20 |
| hlgG1 | −2 | 20 |
| Conjugate 27 | −2 | 100 |
| Conjugate 27 | −24 | 80 |
| Conjugate 27 | −48 | 20 |
| Conjugate 87 | −2 | 100 |
| Conjugate 87 | −24 | 100 |
| Conjugate 87 | −48 | 60 |

### Example 253—Measurement of Binding of ADC Conjugates to LPS Using the Limulus Amebocyte Lysate (LAL) Assay

The LAL assay (Charles River) was used to determine the binding affinity of test articles to LPS. A decrease in absorbance by test articles demonstrates neutralization of LPS and correlates with binding of test articles to LPS.

For the assay, 25 μL purified LPS from E. coli O111:1B4 (Sigma) at doses ranging from 1-10,000 ng/mL were incubated with 25 μL of test article at concentrations ranging from 0.25-10 NM. After 5 min incubation at 37° C., 50 μL of LAL reagent, which reacts with lipid A portion from LPS, was added to corresponding wells. After 15 min, absorbance at 405 nm was read on EnSpire plate reader (Perkin Elmer). The % LPS neutralization is calculated relative to a PBS control (background) and LPS control at each concentration (positive control).

LPS neutralization=(1−(absorbance test article−PBS with LAL reagent)/(absorbance no test article−PBS with LAL reagent))×100

To obtain a single value of LPS neutralization for each test article, the area under the curve (AUC) for dose-response of test articles from 0.25-10 μm for each LPS concentration was calculated using Prism 7 software. The % LPS neutralization is the sum of the AUCs for each LPS concentration determined relative to the maximal AUC achievable×100.

### Results

Fc conjugates bind to purified LPS from E. coli O111:1B4 in a dose-dependent manner at concentrations ranging from 0.25-10 μM and against concentrations of LPS ranging from 1-10.000 ng/mL. A dependence on drug-to-antibody-ratio (DAR) on LPS neutralization was noted—with a higher DAR correlating with tighter binding. LPS neutralization was greater for Fc conjugates compared to TM alone and ranged from above 75% to complete neutralization (100%) for CTC-140 (Table 14).

TABLE 14
LPS neutralization of Fc conjugates and corresponding
TMs against LPS from E. coli
O111:B4

| | | | % LPS | % LPS |
|---|---|---|---|---|
| | | | neutralization | neutralization |
| Test article | TM (DAR) | | of ADC | of TM |
| Conjugate 81 | Int-80 | (9.5) | 99.6 | 55.7 |
| Conjugate 82 | Int-84 | (6.6) | 100 | 53.7 |
| Conjugate 83 | Int-83 | (6.5) | 82.9 | 30.9 |
| Conjugate 86 | Int-94 | (6.6) | 96.36 | 79.3 |
| Conjugate 87 | Int-100 | (6) | 81.9 | 38.9 |
| Conjugate 88 | Int-105 | (3.1) | 77.5 | 33.2 |

| | | | |
|---|---|---|---|
| hlgG1(Myc)Fc | N/A | 1.2 | N/A |

### Example 264—Measurement of Binding of ADC Conjugates to LPS Using the Limulus Amebocyte Lysate (LAL) Assay

The LAL assay (Charles River) was used to determine the binding affinity of test articles to LPS. A decrease in absorbance by test articles demonstrates neutralization of LPS and correlates with binding of test articles to LPS.

For the assay, 25 μL purified LPS from E. coli O111:B4 (Sigma) at doses ranging from 1-10,000 ng/mL were incubated with 25 μL of test article at concentrations ranging from 0.25-10 μM. After 5 min incubation at 37° C., 50 μL of LAL reagent, which reacts with lipid A portion from LPS, was added to corresponding wells. After 15 min, absorbance at 405 nm was read on EnSpire plate reader (Perkin Elmer). The % LPS neutralization is calculated relative to a PBS control (background) and LPS control at each concentration (positive control).

LPS neutralization=(1−(absorbance test article−PBS with LAL reagent)/(absorbance no test article−PBS with LAL reagent))×100

To obtain a single value of LPS neutralization for each test article, the area under the curve (AUC) for dose-response of test articles from 0.25-10 μm for each LPS concentration was calculated using Prism 7 software. The % LPS neutralization is the sum of the AUCs for each LPS concentration determined relative to the maximal AUC achievable×100.

Results

Targeting Moieties (TMs) and Fc conjugates bind to purified LPS from E. coli O111:B4 in a dose-dependent manner at concentrations ranging from 0.25-10 μM and against concentrations of LPS ranging from 1-10,000 ng/mL. LPS neutralization for TMs and Fc conjugates was greater than that for colistin or PMB (Table 15).

TABLE 15
LPS neutralization of TMS and Fc conjugates against
LPS from E. coli O111:B4

| Test article | % LPS neutralization |
|---|---|
| colistin sulfate | 9.21 |
| PMB sulfate | 18.1 |
| higG1 Fc | 1.8 |
| Int-115 | 38.2 |
| Int-116 | 33.2 |
| Int-118 | 59.0 |
| Int-119 | 18 9 |
| Int-120 | 68.0 |
| Int-121 | 32.6 |
| Int-123 | 64.1 |
| Int-124 | 97.1 |
| Int-126 | 19.7 |
| Int-127 | 13.0 |
| Int-129 | 23.0 |
| Conjugate 91 | 84.5 |
| Conjugate 92 | 67.0 |

### Example 255—Measuring Binding of Fc Conjugates to E. coli BioParticles

As a proxy for measuring LPS binding affinities of Fc conjugates to LPS, binding of immobilized Fc conjugates to fluorescein labeled E. coli (K-12 strain, Invitrogen) bioparticles was measured using an ELISA assay. Briefly, Nunc MaxiSorp 98-well plates (ThermoFisher Scientific) were coated overnight at 4° C. with 100 μl/well (1 Ng) test compound at 10 μg/ml in carbonate-bicarbonate buffer (Sigma). The next day, plates were washed 5 times with 1×PBS pH 7.4 (—CaCl₂—MgCl₂; Life Technologies) containing 0.05% Tween-20 (PBST) and blocked with 200 μl 1% BSA Cohn fraction V (Sigma) in 1×PBS pH 7.4 for 1 h at room temperature with shaking (500 rpm). Plates were washed 5 times with PBST before adding 100 ul of E. coli (K-12 strain) BioParticle fluorescein conjugates (Invitrogen) 2-fold diluted in IX PBS (starting at 5 μg/ml), and incubating for 2 h at room temperature with shaking (500 rpm) in the dark. Plates were washed 4 times with PBST and 100 μl 1× PBS added to all wells. Fluorescence was measured with the Perkin-Elmer Enspire Multimode plate reader (excitation fluorescence 490 nm, emission fluorescence 525 nm) and plotted versus concentration using GraphPad Prism software (version 7.0d, GraphPad Software, Inc.). The area under the curve (AUC) was used to rank-order compounds by E. coli binding activity (Table 16).

TABLE 16
Binding of immobilized conjugates to E. coli (K-12 strain) BioParticles
fluorescein conjugate by ELISA

| | | Binding of conjugates to E. coli |
|---|---|---|
| Compound | EM type | (average AUC) |
| Conjugate 80 | hlgG1 | 8.93E+09 |
| Conjugate 81 | hlgG1 | 8.93E+09 |
| Conjugate 82 | hlgG1 | 3.07E+10 |
| Conjugate 83 | hlgG1 | 3.01E+10 |
| Conjugate 85 | hlgG1 | 2.50E+10 |
| Conjugate 86 | hlgG1 | 2.11E+10 |
| Conjugate 87 | hlgG1 | 8.70E+09 |

### Example 256—Measuring LPS Neutralization Using the HEK-Blue hTLR4 Reporter Cell Line

This method measured the ability of compounds to neutralize lipopolysaccharide (LPS) and prevent the downstream activation of NF-κB-signaling mediated by LPS using the HEK-Blue hTLR4 reporter cell line (Fisher Scientific). HEK-Blue hTLR4 cells were cultured to 50-80% confluency in T150 cm² flasks according to the manufacturer's method, then gently rinsed with 20 ml 1× PBS pH 7.4. After aspirating the rinse, 10 ml 1× PBS was added and incubated for 1-2 min at 37° C., 5% CO₂. Cells were released from the flask by tapping the flask or by using a cell lifter. A single cell suspension was obtained by pipetting up and down, then 10 μl of cell suspension added to 80 μl of 1×PBS and 10 μl of trypan blue (0.4% stock solution) for hemocytometer enumeration. The cell concentration was adjusted to 140,000 cells/ml in HEK-Blue Detection medium (Fisher Scientific). 20 μl of test compound at the desired concentration was added to a flat-bottom tissue culture treated 96-well plate (Corning). 20 μl LPS stock (1 mg/md) was added in a dose-response at the desired concentration to corresponding wells. 180 μl of HEK-Blue hTLR4 cells (˜25,000 cells/well) were added to corresponding wells, and the plate incubated at 37° C., 5% CO₂ for 6-16 h. The absorbance at 650 nm was measured using a Perkin-Elmer Enspire Multimode plate reader. Percent LPS inactivation (1-[absorbance test article with LPS/absorbance no test article with LPS]×100%) was plotted versus concentration using GraphPad Prism software (version 7.0d, GraphPad Software, Inc.). The area under the curve (AUC) was used to rank-order compounds by LPS neutralizing activity (Table 17).

TABLE 17
E. coli O111:B4 LPS neutralization activity of compounds as
measured by inhibition of NF-κB-signaling mediated
by LPS in HEK-Blue hTLR4 reporter cells.

| | | NF-κB inhibition in HEK- | |
|---|---|---|---|
| | | Blue hTLR4 cells (average | |
| | | AUC normalized to colistin) | |
| | | | LPS |
| Compound | Description | TM type | 109 ng/ml |
| Conjugate 85 | hlgG1 | monomer | 104.3 |
| Conjugate 86 | hlgG1 | monomer | 124.8 |
| Conjugate 87 | hlgG1 | monomer | 165.4 |

### Example 257—LPS Neutralization Activity of Compounds in Human Blood

The ability of compounds to sequester LPS and inhibit immune cell activation in human blood was measured to rank order LPS-binding affinities, and demonstrate that test articles prevent LPS mediated immune activation. Hirudin anti-coagulated human blood (complement active) was diluted 1:2 in in RPMI with L-glutamine and phenol red (Life Technologies) and 160 μl added to wells of a non-binding 96-well plate (Corning 3641). 10 μl of 20× compound diluted in RPMI (2.5-0.0006 μM final concentration) was added to wells, followed by 10 μl of 20× LPS from E. coli O111:B4 (Sigma; 100 or 1,000 ng/ml final concentration), and 20 μl RPMI for a final blood concentration of 40%. The plate was incubated under static conditions at 37° C. for 4 h, centrifuged at 500×g for 5 min at room temperature, and 60 μl plasma carefully aspirated and transferred to a non-binding plate for overnight storage at −80° C. The following day, the concentration of TNF-α in plasma samples diluted 1:10 was determined using the human TNF-α ELISA kit, as per the manufacturer's instructions (BioLegend). The percent inhibition of TNF-α release, relative to the blood+LPS positive control (100% TNF-α release), was calculated and plotted for each compound concentration tested. The area under the curve (AUC) was calculated using GraphPad Prism software (version 7.0d, GraphPad Software, Inc.) and used to rank-order compounds by LPS neutralizing activity. LPS stimulation of whole blood is known to trigger the release of pro-inflammatory cytokine TNF-α (PMID: 26993088). Therefore, high levels of TNF-α inhibition, resulting in high AUCs, were indicative of compounds with good LPS neutralizing activity in human blood (Table 18).

TABLE 18
E. coli O111:B4 LPS neutralization activity of compounds
in human blood as measured by the
inhibition of TNF-α release.

| | | | TNF-α inhibition in human | |
|---|---|---|---|---|
| | | | blood (average AUC | |
| | | | normalized to colistin | |
| | | | LPS | LPS |
| Compound | Description | TM type | 100 ng/ml | 1,000 ng/ml |
| Conjugate 81 | hlgG1 | monomer | 177.97 | 177.98 |
| Conjugate 82 | hlgG1 | monomer | 173.9 | 770.4 |
| Conjugate 83 | hlgG1 | monomer | 168.1 | 741.1 |
| Conjugate 85 | hlgG1 | monomer | 92.8 | 1022.2 |
| Conjugate 86 | hlgG1 | monomer | 145.2 | 2170.0 |
| Conjugate 87 | hlgG1 | monomer | 102.72 | 901.88 |

### Example 258—Antibacterial Activity of Compounds in 40% Human Blood

A comparison of the minimum bactericidal concentrations (MBC) of Fc-conjugates in the presence of complement active human blood versus their minimum inhibitory concentrations in standard media demonstrate that Fc-conjugates potentiate immune mediated bacterial killing, likely through complement recruitment. Overnight stationary-phase cultures were centrifuged at 3,500×g for 10 min at room temperature and resuspended in 1×PBS to an optical density at 600 nm (OD₆₀₀) of 0.4 (1×10⁸ colony forming units [CFU]/ml in RPMI with L-glutamine and phenol red (Life Technologies). Bacteria were diluted 1:25 in RPMI (4×10⁸ CFU/ml) and 10 μl added to wells of a non-binding 96-well plate (Corning 3841) containing 80 μl of 50% hirudin anti-coagulated blood (complement active) in RPMI. After adding 5 μl of 20× compound diluted in RPMI (TMs, 0.2-0.0031 μM final concentration; Fc conjugates, 64-1 μg/ml final concentration), and 5 NI RPMI for a final blood concentration 40%, the plate was incubated under static conditions at 37° C. for 90 min MBC was determined by 10-fold serial dilution in 1× PBS, then plating 4 μl spots onto rectangular LB agar plates and overnight incubation at 37° C. MBC was defined as the minimum compound concentration required for complete bacterial clearance (Table 18).

Table 19 shows the minimum bactericidal concentration (MBC) in human blood for polymyxin-Fc conjugate (μg/ml) for Gram-negative pathogenic bacteria E. coli 25922, A. baumannii 5075 and P. aeruginosa 01. Minimum inhibitory concentration (MIC) in RPMI 1640 cell culture medium pH 7.4, with bicarbonate and 5% LB are included for comparison. In most cases, 2-4 fold improvements in antibacterial activity for the conjugates are observed in the presence of complement active human blood.

TABLE 19
Minimum bactericidal concentration (MBC) in human blood

| | MBC in blood (μg/mL) | | |
|---|---|---|---|
| | MIC in RPMI (μg/mL) | | |
| Compound | EC 25922 | AB 5075 | PA 01 |
| Conjugate 82 | 32 | 32 | >64 |
| | 128 | 64 | >256 |
| Conjugate 83 | 64 | 64 | >64 |
| | 256 | 256 | >256 |
| Conjugate 85 | >64 | 64 | >64 |
| | >256 | 128 | >256 |
| Conjugate 86 | 64 | 16 | >64 |
| | 256 | 64 | >256 |
| Conjugate 87 | >64 | >64 | >64 |
| | >256 | 256 | >256 |
| Conjugate 88 | 32 | 8 | >64 |
| | 256 | 32 | >256 |
| Colistin | 0.025 | 0.05 | 0.2 |
| | 0.39 | 0.79 | 1.58 |
| PMB | 0.0125 | 0.025 | 0.2 |
| | <0.18 | 0.36 | 1.44 |
| Int-115 | 0.05 | 0.05 | >0.2 |
| | 2.23 | 1.11 | 8.91 |
| Int-116 | 0.2 | 0.05 | >0.2 |
| | 1.05 | 1.05 | 8.41 |
| Int-117 | >0.2 | >0.2 | >0.2 |
| | 1.08 | 1.08 | 2.16 |
| Int-118 | 0.05 | 0.025 | >0.2 |
| | 0.95 | 0.95 | 3.81 |
| Int-119 | 0.1 | 0.05 | >0.2 |
| | 1.94 | 0.97 | 7.78 |
| Int-120 | 0.025 | 0.025 | >0.2 |
| | 0.6 | 0.6 | 4.8 |
| Int-121 | 0.0125 | 0.0125 | >0.2 |
| | 0.56 | 0.56 | 4.51 |
| Int-122 | 0.0125 | 0.0125 | >0.2 |
| | 0.55 | 0.55 | 2.19 |
| Int-123 | 0.025 | 0.0125 | >0.2 |
| | 1.11 | 1.11 | 2.22 |
| Int-124 | 0.0125 | 0.0125 | 0.2 |
| | 0.99 | 0.99 | 1.99 |
| Int-125 | 0.05 | 0.1 | 0.2 |
| | 0.94 | 1.88 | 3.76 |
| Int-127 | 0.025 | 0.1 | 0.1 |
| | 1.00 | 1.00 | 3.99 |
| Int-129 | 0.05 | 0.05 | >0.2 |
| | 1.00 | 0.05 | >0.2 |
| hlgG1 Fc | >64 | >64 | >64 |
| | >512 | >512 | >512 |
| Conjugate 90 | >64 | 32 | >64 |
| | >512 | 256 | >512 |
| Conjugate 91 | 4 | 16 | >64 |
| | 128 | 32 | >512 |
| Conjugate 92 | 8 | 4 | >64 |
| | 64 | 32 | >512 |
| Conjugate 93 | 8 | 32 | >64 |
| | 64 | 32 | >512 |
| Conjugate 94 | 16 | 8 | >64 |
| | 256 | 32 | >512 |
| Conjugate 95 | 32 | 32 | >64 |
| | >256 | 256 | >256 |
| Conjugate 96 | 64 | >64 | >64 |
| | >512 | >512 | >512 |

Example 259—Binding of Conjugates 62, 83, and 66 to Bacteria Measured by Flow Cytometry Binding of conjugates to gram negative bacteria was measured via flow cytometry. Gram negative bacteria (E. coli ATCC 25922) were grown overnight in Luria-Bertani (LB) (BD Difco™ Cat. 244620) at 37° C. while shaking. The following day, bacteria were grown to log-phase in LB until reaching an OD₆₀₀ of 0.4 (˜10e CFU/mL). 1 mL of bacteria per condition were washed in DPBS pH 7.4 (Gibco 10010-023), resuspended in RPMI (Gibco 11875-093) and 1×10⁵ bacteria were plated per well in a 96-well, untreated, round-bottom plate (Corning 3788). Fc-conjugates or the negative control (hIgG1-Fc) were added at 5 μM and plates were incubated with agitation at 37° C. for 15 minutes. Conjugate 82 (FIG. 113A), Conjugate 83 (FIG. 113B) and Conjugate 88 (FIG. 113C) (shown as open, black histograms in FIGS. 113A-113C) or control molecule hIgG-Fc (shown as filled, gray histograms in FIGS. 113A-113C) for 15 minutes. Bacteria were washed and molecules bound to the surface of the bacteria were detected via flow cytometry using the appropriate fluorescently-conjugated secondary antibodies. Specifically, bacteria were washed 1× in DPBS stained with Zombie NIR fixable viability dye (Biolegend 423105) in PBS for 15 minutes on ice. Bacteria were then washed 1× in staining buffer (20 mM HEPES (Gibco 15630-080)+1% Bovine Serum Albumin (Sigma A5611) in DPBS). Bound hIgG-Fc conjugates were detected by staining with an anti-myc-FITC labeled antibody at 1:50 (Abcam 1394) for 30 minutes on ice in the dark. Finally, bacteria were washed 1× in DPBS and fixed in a 2% solution of paraformaldehyde/DPBS. 50,000 events/sample were acquired on a BD FACS Calibur and analysis was performed using FlowJo software. Viable bacteria were gated as NIR dye negative based on RedFL2 vs SSC. hIgG-Fc conjugate binding was detected as fluorescence in the FITC channel. The data shown in FIGS. 113A-113C demonstrate that Conjugates 82, 83, and 88 tightly bind to lipopolysaccharides on the surface of E. coli ATCC 25922.

### Example 260—Protective Effect of Stabilized Dimers in a Lethal Model of Endotoxemia (Conjugates 88 and 89)

Sensitized mouse model of endotoxemia. These studies used female immune competent C57BL/6 mice (Jackson Laboratories) between 8 and 10 weeks old. After acclimation for 7 days mice were challenged IP with 18 ng of purified LPS (Escherichia coli 0111:B4; Sigma #L4130) in PBS mixed with 20 mg of D-galactosamine hydrochloride (Fisher #50-492-412) in a volume of 0.4 ml. Previously this dose was shown to result in 100% mortality of challenged mice within 5 days. The murine LD₁₀₀ of this model is approximately equal to that of humans due to sensitization by D-galactosamine. Study mice were housed and cared for following standard IACUC approved protocols, and clearly moribund mice were humanely euthanized and scored as a death.

Efficacy of stabilized dimers in endotoxemia. C57BL/6 mice were challenged with a lethal dose of LPS as described above. One hour prior to LPS challenge control groups of animals received vehicle (PBS), colistin at 1 mg/kg, or the indicated ADC at 25, 5, or 1 mg/kg by IV injection. As illustrated in Table 20, the majority of mice dosed with vehicle (PBS) or Fc alone (hIgG1) reached mortality within 24 hours of LPS challenge. In contrast, Conjugate 88 was 100% protective at 25 mg/kg and Conjugate 89 was fully protective down to 5 mg/kg. These studies demonstrates the LPS neutralizing capabilities of stabilized dimers when dosed by IV.

TABLE 20
LPS Range

| Test article | Dose (mg/kg) | % Survival |
|---|---|---|
| PBS | na | 20 |
| hlgG1 | 25 | 0 |
| Conjugate 88 | 25 | 100 |
| Conjugate 89 | 5 | 100 |

* = 24 hour time point.

### Example 261—Activity of Compounds in Bacteremia Mouse Model

In vivo screening of selected compounds was accomplished in an E. coli bacteremia model (N=3). Efficacy was assessed in an animal model of infection using CD-1 mice by evaluating bacteremia in immune competent mice challenged with 2× the LD₉₅ of E. coli 25922 (dosed qd, intraperitoneally (IP) at T+1 hour, survival endpoint).

MICs for compounds against the challenge strain are shown with and without serum. Colistin (COL) and Int-17 were dosed at 0.3 mg/kg and Int-41 was dosed at 3.0 mg/kg. Percent survival was determined 72 hours after bacterial challenge. Compounds with MICs against E. coli 25922 of less than 4 μg/ml were evaluated in the bacteremia model. As indicated in Table 21, compounds were significantly more active in the presence of serum. This serum synergy translated into potent activity in this model with most compounds achieving 100% efficacy.

TABLE 21

| | MIC (μg/ml) | | |
|---|---|---|---|
| | E. coli ATCC 25922 | | % Survival (IP, QD) |
| Test article | No serum | Serum | 0.3 or 3.0 mg/kg |
| Vehicle (PBS) | na | na | 0 |
| COL | 0.5 | 0.25 | 100 |
| Int-17 | 2 | 0.5 | 100 |
| Int-41 | 4 | 1 | 67 |

### Example 262. Testing of Compounds in Mouse Efficacy Models

Int-115 and Int-118 were evaluated for potency in a lethal model of bacteremia. Briefly, immune competent CD-1 mice (N=3, Charles River Laboratories) between 20-22 gm were challenged with approximately 2× the LD₉₀ of E. coli 25922 (in 5% gastric hog mucin) by intraperitoneal (IP) injection. Test articles were dissolved in 1× PBS and injected IV at a dose volume of 10 ml/kg one hour following bacterial challenge. Animals were monitored twice daily and mortality scored over three days. As shown in Table 22, both compounds demonstrated efficacy in the E. coli bacteremia model comparable with colistin. Additionally no adverse effects were observed in the animals when dosed at these concentrations.

Studies (B)-(D) shown in Table 22 summarizes the efficacy of compounds in lethal models of endotoxemia. In these models 6 week old C57I/6J immune competent mice (Jackson Laboratory) were challenged IP with purified lipopolysaccharide (LPS) in PBS at an LD₉₅. For all studies, LPS was obtained from Sigma (catalog numbers in the table footnote) or were purified using a kit obtained from iNtRON Biotechnology Inc. (cat #17141). One hour prior to LPS challenge mice were dosed with test articles as indicated (Table 22). The results of these studies indicate that compounds are able to neutralize the lethal effects of LPS in a severe endotoxemia/sepsis model. Importantly the compounds are able to neutralize multiple LPS types and this observation does not appear to be compound specific since Int-107 is able to neutralize LPS from Neisseria and COL^{R} E. coli.

TABLE 22
Testing of select compounds in bacteremia and endotoxemia models.

| | | Dose | | | |
|---|---|---|---|---|---|
| Model | Test | | | Time | % |
| (Lethal agent) | Article | mg/kg | Route/Frequency | (hr) | survival |
| (A) Bacteremia | COL | 5 | IV, single | +1 | 66 /100* |
| (E. coli) | Int-115 | 10 | IV, single | +1 | 100 |
| | Int-118 | 5 | IV, single | +1 | 66 |
| (B) Endotoxemia | COL | 1 | IV, single | −1 | 100 |
| (Ec LPS) | Int-129 | 1 | IV, single | −1 | 100 |
| (C) Endotoxemia | COL | 5 | IV, single | −1 | 40 |
| (Ng LPS) | Int-107 | 5 | IV, single | −1 | 100 |
| (D) Endotoxemia | COL | 5 | IV, single | −1 | 0 |
| (Ec COL^{R} LPS) | Int-107 | 5 | IV, single | −1 | 80 |
| | Int-115 | 5 | IV, single | −1 | 100 |
| | Int-120 | 5 | IV, single | −1 | 100 |
| | Int-122 | 5 | IV, single | −1 | 80 |

CD-1 mice were used for bacteremia, C57BL/6J mice for endotoxemia. The endpoint was survival after three days for both models. Ec: E. coli 0111:B34 (Sigma cat #L4130); Ng: Neisseria gonorrhoeae 19424 (iNtRON cat #17141); Ec COL^{R}: LPS from a colistin-resistant (MCR-1) strain (AR0349) (iNtRON cat #17141); COL: colistin. *Data from two separate studies.

### Example 263. Testing of Conjugates in Mouse Endotoxemia Models

Murine endotoxemia studies were conducted as described previously in C57BL/6J mice challenged with an LD₉₅ of purified LPS (see Example 262. Table 23 (A) highlights the ability of multiple conjugates to protect against lethal challenge with LPS from E. coli. The dose listed is the lowest which resulted in 80-100% protection. Lower doses of Conjugate 91 and Conjugate 92 were equivalent to colistin in terms of efficacy.

Protection was also achieved by several different dosing mutes (Table 23), including IV, IP, and SC. These data (B) and (C) also demonstrate the ability of conjugates to neutralize LPS isolated from additional, diverse Gram-negative species.

TABLE 23
Testing of select ADCs in endotoxemia models.

| | | Dose | | | |
|---|---|---|---|---|---|
| Model | Test | | Route/ | Time | % |
| (Lethal agent) | Article | mg/kg | Frequency | (hr) | survival |
| (A) Endotoxemia | COL | 5 | IV, single | −1 | 100 |
| (Ec LPS) | Conjugate 91 | 1 | IV, single | −1 | 100 |
| | Conjugate 91 | 10 | SC, single | −1 | 100 |
| | Conjugate 91 | 1 | IP, single | −1 | 100 |
| | Conjugate 92 | 3 | IV, single | −1 | 100 |
| | Conjugate 93 | 10 | IV, single | −1 | 100 |
| | Conjugate 94 | 10 | IV, single | −1 | 100 |
| | Conjugate 95 | 10 | IV, single | −1 | 80 |
| | Conjugate 96 | 10 | IV, single | −1 | 80 |
| (B) Endotoxemia | COL | 5 | IV, single | −1 | 20 |
| (Ab LPS) | Conjugate 92 | 25 | IV, single | −1 | 100 |
| (C) Endotoxemia | COL | 5 | IV, single | −1 | 40 |
| (Ng LPS) | Conjugate 90 | 50 | IV, single | −1 | 80 |

End point was survival after 3 days. Ec: E. coli 0111:1B4 (Sigma cat #L4130); Ab: Acinetobacter baumannii 5075 (iNtRON cat #17141); Ng: Neisseria gonorrhoeae 19424 (iNtRON cat #17141); COL: colistin.

### Example 264. Extended Testing of ADCs Against Multiple LPS Types in Mouse Endotoxemia Models

Due to the diverse types of Gram-negative pathogens capable of generating septic infections (endotoxemia) it was demonstrated that conjugates could recognize and neutralize multiple LPS types. These endotoxemia studies were conducted as described previously in C57BL/6J mice (see Example 262 by challenging them with an LD₉₅ of purified LPS. Conjugate 41 was active against LPS from an E. coli strain with an 0111:1B4 LPS type while the Fc-only control showed no protection (Table 24, (A)). Study (D) expanded this observation and showed that Conjugate 41 also neutralized LPS from an E. coli 026:1B6 strain. Critically, this data also demonstrates that conjugates provide complete protection from LPS isolated from strains of K. pneumoniae and P. aeruginosa, two clinically important pathogens.

These data also demonstrate that conjugates are effective at neutralizing LPS from COL^{R} strains (E. coli AR0349). The colistin-resistant phenotype of this strain is known to be mediated by modification of its LPS, therefore the ability to neutralize this LPS while colistin does not, further supports the superior activity of the conjugates. Lastly, protection was demonstrated against challenge by Proteus mirabilis LPS (40%, Table 24, (E)) death was significantly delayed when animals were treated with Conjugate 92 (see footnote).

TABLE 24
Host range testing of ADCs in endotoxemia
models with various LPS types.

| Model | Test | | Route/ | Time | % |
|---|---|---|---|---|---|
| (Lethal agent) | Article | mg/kg | Frequency | (hr) | survival |
| A) Endotoxemia | COL | 5 | IV, bid × 1 | −1 | 80 |
| (Ec 0111:B4 LPS) | Fc only | 25 | IV, single | −1 | 0 |
| | Conjugate | 25 | IV, single | −1 | 100 |
| | 41 | | | | |
| B) Endotoxemia | Conjugate | 25 | IV, single | −1 | 100 |
| (Ab LPS) | 92 | | | | |
| C) Endotoxemia | Conjugate | 25 | IV, single | −1 | 100 |
| (Kp LPS) | 23 | | | | |
| D) Endotoxemia | Conjugate | 25 | IV, single | −1 | 100 |
| (Ec 026:B6 LPS) | 41 | | | | |
| E) Endotoxemia | Fc only | 25 | IV, single | −1 | 0 |
| (Pm LPS) | Conjugate | 25 | IV, single | −1 | 40* |
| | 41 | | | | |
| F) Endotoxemia | Conjugate | 25 | IV, single | −1 | 80 |
| (Sm LPS) | 41 | | | | |
| G) Endotoxemia ) | Conjugate | 25 | IV, single | −1 | 100 |
| (Pa LPS | 41 | | | | |
| H) Endotoxemia | COL | 5 | IV, single | −1 | 20 |
| (Ec COL^{R} LPS) | Conjugate | 25 | IV, single | −1 | 100 |
| | 92 | | | | |

End point was survival after 3 days. Ec: E. coli 0111:B34, Sigma cat #L4130; Ab: Acinetobacter baumannii 5075; Kp: Klebsiella pneumonia 10, Sigma cat #L4268; Ng: Neisseria gonorrhoeae 19424; Ec 026:B86, Sigma cat #L2762; Pm: Proteus mirabilis, Sigma cat #SMB00704; Sm: Serratia marcescens, Sigma cat #L6016; Pa: Pseudomonas aeruginosa 10. Sigma cat #L8643; Ec COL^{R}: LPS from a colistin-resistant (MCR-1) strain (AR0349). Purified LPS used for all endotoxemia studies and if not denoted by a Sigma catalog number the LPS was purified in-house using an iNtRON #17141 purification kit. *Delayed death, at the time Fc only animals reached 100% mortality all Conjugate 41 mice were still alive.

### Example 265. Compound Activity in Mouse Models of Acinetobacter Baumannii Pneumonia

All animal studies were performed under protocols approved by the UCSD Institutional Animal Use and Care Committee.

The murine pneumonia model was adapted from Lin et al., 2015 (PMID: 26288841). A. baumannii AB5075 was grown to log-phase in LB at 37° C., 200 rpm shaking. Bacteria were washed once with PBS via centrifugation at 4,000 rpm. Bacteria were adjusted to desired concentration in PBS.

Prior to infection, 8-week-old female C57BI/6J mice (Jackson Labs) were anesthetized with 100 mg/kg ketamine and 10 mg/kg xylazine. Using an otoscope, mice were infected into the trachea during inspiration with 1×10⁸ CFU in 30 μL of PBS.

The survival (n=5 per group) was monitored twice daily for 5 days. Mice were treated twice intravenously with 5 mg/kg (FIG. 118) (colistin, Int-116, or Int-118) or 10 mg/kg (FIG. 119) (colistin, Int-115, or Int-129) or PBS as control at t=+1 h and +4 h post-infection, at t=+1 h and 4 h post-infection. A single treatment with Fc conjugate (Conjugate 91) or human IgG1 Fc as control was administered with concentration ranging from 10-75 mg/kg subcutaneously at t=−24 h prior to infection.

Int-116 was fully protective and Int-118 was 40% protective against lethal challenge with A. baumannii 5075. In comparison, colistin used as positive control, was 80% protective and all mice succumbed to infection in the group treated with negative control PBS (FIG. 118).

Int-129 was protective showing 60% survival. Int-115 was able to delay death of all mice until day 4 post-infection. All mice treated with PBS as control succumbed to infection at day 5 (FIG. 119).

Conjugate 91 at 75 mg/kg showed 20% protection at day 5 post-infection, whereas lower concentration or CTP-143 control showed no protection. At 50 mg/kg of Conjugate 91 a delay in death of 0.5 days was observed (FIG. 120).

### OTHER EMBODIMENTS

While the disclosure has been described in connection with specific embodiments thereof, it will be understood that it is capable of further modifications and this application is intended to cover any variations, uses, or adaptations of the disclosure following, in general, the principles of the disclosure and including such departures from the present disclosure come within known or customary practice within the art to which the disclosure pertains and may be applied to the essential features hereinbefore set forth.

All publications, patents and patent applications are herein incorporated by reference in their entirety to the same extent as if each individual publication, patent or patent application was specifically and individually indicated to be incorporated by reference in its entirety.

What is claimed is:

1-34. (canceled)

35. A conjugate described by formula (D-Ia): 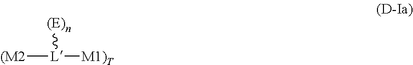
wherein each M1 comprises a first cyclic heptapeptide comprising a linking nitrogen and each M2 comprises a second cyclic heptapeptide comprising a linking nitrogen;
L′ in each M2-L′-M1 is a linker covalently attached to the Fc domain monomer and to the linking nitrogen in each of M1 and M2;
each E is an Fc domain monomer;
n is 1 or 2; and
T is an integer from 1 to 15,
or a pharmaceutically acceptable salt thereof.

36. (canceled)

37. The conjugate of claim 35, wherein L′ in each M2-L′-M1 is described by formula (D-L): 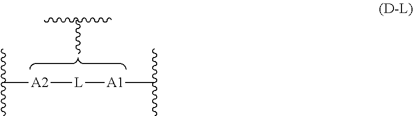
wherein L is a remainder of L′;
A1 is a 1-5 amino acid peptide covalently attached to the linking nitrogen in each M1 or is absent; and
A2 is a 1-5 amino acid peptide covalently attached to the linking nitrogen in each M2 or is absent.

38. The conjugate of claim 35, wherein the conjugate is described by formula (D-IIa): 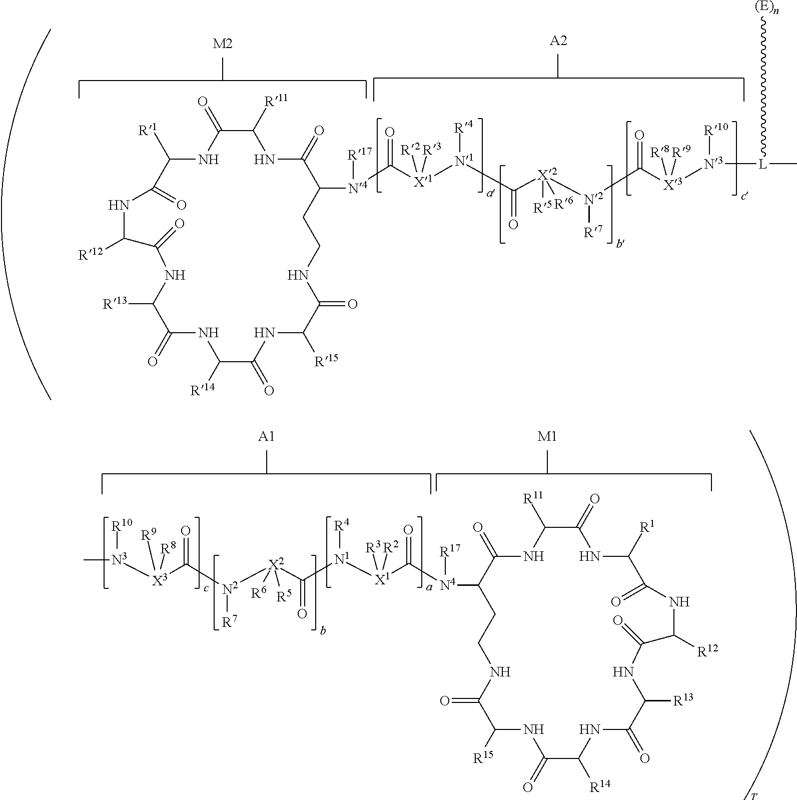
wherein L is a remainder of L′;
each of R¹, R¹², R′¹, and R′¹² is, independently, a lipophilic moiety, a polar moiety, or H;
each of R¹¹, R¹³, R¹⁴, R′¹¹, R′¹³, and R′¹⁴ is, independently, optionally substituted C1-C5 alkamino, a polar moiety, a positively charged moiety, or H;
each of R¹⁵ and R′¹⁵ is, independently, a lipophilic moiety or a polar moiety;
each of R², R³, R⁴, R⁵, R⁶, R⁷, R⁸, R⁹, R¹⁰, R′², R′³, R′⁴, R′⁵, R′⁶, R′⁷, R′⁸, R′⁹, and R′¹⁰ is, independently, a lipophilic moiety, a positively charged moiety, a polar moiety, H, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; or two R groups on the same or adjacent atoms join to form an optionally substituted 3-8 membered ring;
each or R¹⁷ and R′¹⁷ is, independently, H, C1-C3 alkyl, or C2-C3 alkamino;
each of a′, b′, c′, a, b, and c is, independently, 0 or 1;
each of N¹, N², N³, N⁴, N′¹, N′², N′³, and N′⁴ is a nitrogen atom; and
each of X¹, X², X³, X′¹, X′², and X′³ is, independently, a carbon atom or a nitrogen atom,
wherein if X¹ is a nitrogen atom then R³ is absent, if X′¹ is a nitrogen atom then R′³ is absent, if X² is a nitrogen atom then R⁵ is absent, if X′² is a nitrogen atom then R′⁵ is absent, if X³ is a nitrogen atom then R⁹ is absent, and if X′³ is a nitrogen atom then R′⁹ is absent;
or a pharmaceutically acceptable salt thereof.

39-104. (canceled)

105. The conjugate of claim 38, wherein the conjugate is described by formula (D-VI): 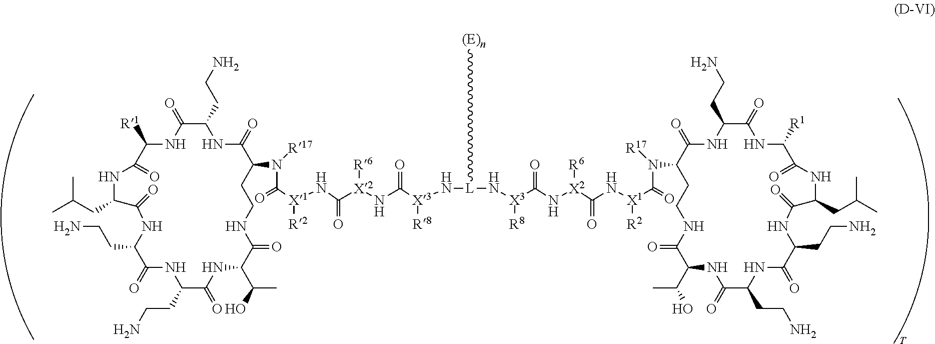
wherein each of R¹ and R′¹ is, independently, optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; and
each of R², R⁶, R⁸, R′², R′⁶, and R′⁸ is, independently, H, a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl,
or a pharmaceutically acceptable salt thereof.

106-229. (canceled)

230. The conjugate of claim 37, wherein L′, L, or L¹ comprises one or more optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, optionally substituted C2-C15 heteroarylene, O, S, NRⁱ, P, carbonyl, thiocarbonyl, sulfonyl, phosphate, phosphoryl, or imino,
wherein Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C3-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl.

231-235. (canceled)

236. The conjugate of claim 37, wherein each L is described by formula (D-L-I): 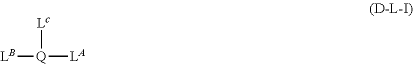
wherein L^{A} is described by formula G^{A1}-(Z^{A1})_{g1}-(Y^{A1})ₕ₁-(Z^{A2})ᵢ₁-(Y^{A2})ⱼ₁-(Z^{A3})ₖ₁-(Y^{A3})ₗ₁-(Z^{A4})ₘ₁-(Y^{A4})ₙ₁-(Z^{A5})_{O}_{₁}-G^{A2};
L^{B} is described by formula G^{B1}-(Z^{B1})_{g2}-(Y^{B1})ₕ₂-(Z^{B2})ᵢ₂-(Y^{B3})ⱼ₂-(Z^{B3})ₖ₂-(Y^{B3})ₗ₂-(Z^{B4})ₘ₂-(Y^{B4})ₙ₂- (Z^{B5})ₒ₂-G²;
L^{C} is described by formula G^{C1}-(Z^{C1})_{g3}-(Y^{C1})ₕ₃-(Z^{C2})ᵢ₃-(Y^{C2})ⱼ₃-(Z^{C3})ₖ₃-(Y^{C3})ₗ₃-(Z^{C4})ₘ₃-(Y^{C4})ₙ₃- (Z^{C5})_{O}_{₃}-G^{C2};
G^{A1} is a bond attached to Q in formula (D-L-I);
G^{A2} is a bond attached to A1 or M1 if A1 is absent;
G^{B1} is a bond attached to Q in formula (D-L-I);
G^{B2} is a bond attached to A2 or M2 if A2 is absent;
G^{C1} is a bond attached to Q in formula (D-L-I);
G^{C2} is a bond attached to E;
each of Z^{A1}, Z^{A2}, Z^{A3}, Z^{A4}, Z^{A5}, Z^{B1}, Z^{B2}, Z^{B3}, Z^{B4}, Z^{B5}, Z^{C1}, Z^{C2}, Z^{C3}, Z^{C4}, and Z^{C5} is, independently, optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene;
each of Y^{A1}, Y^{A2}, Y^{A3}, Y^{A4}, Y^{B1}, Y^{B2}, Y^{B3}, Y^{B4}, Y^{C1}, Y^{C2}, Y^{C3}, and Y^{C4} is, independently, O, S, NRⁱ, P, carbonyl, thiocarbonyl, sulfonyl, phosphate, phosphoryl, or imino;
Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C3-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl;
each of g1, h1, i1, j1, k1, l1, m1, n1, o1, g2, h2, i2, j2, k2, l2, m2, n2, o2, g3, h3, i3, j3, k3, l3, m3, n3, and o3 is, independently, 0 or 1; and
Q is a nitrogen atom, optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene.

237-298. (canceled)

299. The conjugate of claim 35, wherein E has the sequence of any one of SEQ ID NOs: 1-31.

300. The conjugate of claim 35, wherein when n is 2, E dimerizes to form an Fc domain.

301-461. (canceled)

462. A conjugate described by formula (M-Ia): 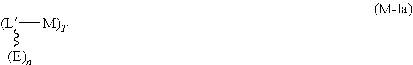
wherein each M comprises a cyclic heptapeptide comprising a linking nitrogen atom;
each E is an Fc domain monomer;
L′ in each L′-M is a linker, wherein L′ is covalently attached to the linking nitrogen atom in M and to E;
n is 1 or 2; and
T is an integer from 1 to 15;
or a pharmaceutically acceptable salt thereof.

463. (canceled)

464. The conjugate of claim 462, wherein L′ in each L′-M is described by formula (M-L′): 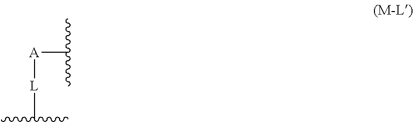
wherein L is a remainder of L′; and
A is 1 to 5 amino acids, wherein at least one A is covalently attached to the linking nitrogen atom in M, or a pharmaceutically acceptable salt thereof.

465. The conjugate claim 462, wherein the conjugate is described by formula M-II): 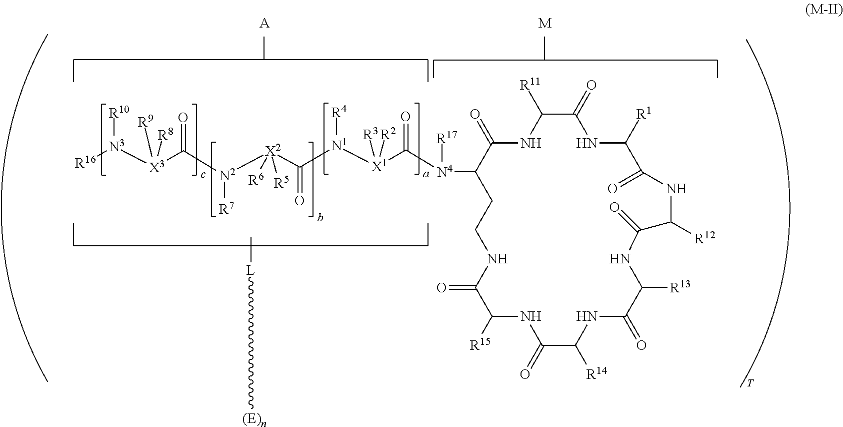
wherein each L is a remainder of L′ and is covalently attached to or may take the place of R², R³, R⁴, R⁵, R⁶, R⁷, R⁸, R⁹, R¹⁰, or R¹⁶;
each of R¹ and R¹² is, independently, a lipophilic moiety, a polar moiety, or H;
each of R¹¹, R¹³, and R¹⁴ is, independently, optionally substituted C1-C5 alkamino, a polar moiety, a positively charged moiety, or H;
each R¹⁵ is a lipophilic moiety or a polar moiety;
each of R², R³, R⁴, R⁵, R⁶, R⁷, R⁸, R⁹, and R¹⁰ is, independently, a lipophilic moiety, a positively charged moiety, a polar moiety, H, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl; or two R groups on the same or adjacent atoms join to form an optionally substituted 3-8 membered ring;
each R¹⁶ is H, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl, or is absent;
R¹⁷ is H, C1-C3 alkyl, or C2-C3 alkamino;
each of a, b, and c is, independently, 0 or 1; and
each of X¹, X², and X³ is a carbon atom or a nitrogen atom,
wherein if X¹ is a nitrogen atom then R³ is absent, if X² is a nitrogen atom then R⁵ is absent, and if X³ is a nitrogen atom then R⁹ is absent;
or a pharmaceutically acceptable salt thereof.

466-567. (canceled)

568. The conjugate of claim 465, wherein the conjugate is described by formula (M-XV): 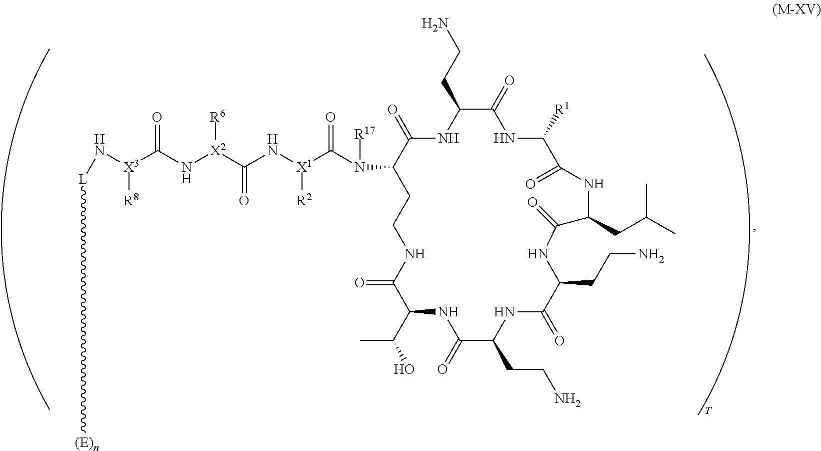
wherein each R¹ is optionally substituted benzyl, CH₂CH(CH₃)₂, cyclopropylmethyl, cyclopentylmethyl, cyclohexylmethyl, naphthylmethyl, optionally substituted thiophenylmethyl, or optionally substituted furanylmethyl; and
each of R², R⁶ and R⁸ is, independently, H, a positively charged moiety, a polar moiety, optionally substituted C1-C5 alkamino, optionally substituted C1-C20 alkyl, optionally substituted C3-C20 cycloalkyl, optionally substituted C4-C20 cycloalkenyl, optionally substituted C8-C20 cycloalkynyl, optionally substituted C5-C15 aryl, optionally substituted C1-C20 heteroalkyl, optionally substituted C3-C20 heterocycloalkyl, optionally substituted C4-C20 heterocycloalkenyl, optionally substituted C8-C20 heterocycloalkynyl, optionally substituted C2-C15 heteroaryl, optionally substituted C6-C35 alkaryl, or optionally substituted C6-C35 heteroalkaryl;
or a pharmaceutically acceptable salt thereof.

569-664. (canceled)

665. The conjugate of claim 462, wherein when n is 2, E dimerizes to form an Fc domain.

666. The conjugate of claim 464, wherein:
each L′ or L comprises one or more optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, optionally substituted C2-C15 heteroarylene, O, S, NRⁱ, P, carbonyl, thiocarbonyl, sulfonyl, phosphate, phosphoryl, or imino; and
Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C3-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl;
or a pharmaceutically acceptable salt thereof.

667-671. (canceled)

672. The conjugate of claim 464, wherein each L is described by formula (M-L-1):
J¹-(Q¹)_{g}-(T¹)ₕ-(Q²)ᵢ-(T²)ⱼ-(Q³)ₖ-(T³)ₗ-(Q⁴)ₘ-(T⁴)ₙ-(Q⁵)ₒ-J² (M-L-1)
wherein:
J¹ is a bond attached to A or M if A is absent;
J² is a bond attached to E;
each of Q¹, Q², Q³, Q⁴, and Q⁵ is, independently, optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene;
each of T¹, T², T³, T4 is, independently, O, S, NRⁱ, P, carbonyl, thiocarbonyl, sulfonyl, phosphate, phosphoryl, or imino;
Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C3-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl; and
each of g, h, i, j, k, l, m, n, and o is, independently, 0 or 1;
or a pharmaceutically acceptable salt thereof.

673-824. (canceled)

825. A pharmaceutical composition comprising a conjugate of claim 35, or a pharmaceutically acceptable salt thereof, and a pharmaceutically acceptable excipient.

826-832. (canceled)

833. A method of;
(a) protecting against or treating a bacterial infection in a subject, said method comprising administering to said subject (1) a conjugate of claim 35 and (2) an antibacterial agent; or
(b) of preventing, stabilizing, or inhibiting the growth of bacteria, or killing bacteria, comprising contacting the bacteria or a site susceptible to bacterial growth with (1) a conjugate of claim 35 and (2) an antibacterial agent.

834-867. (canceled)

868. The conjugate of claim 462, wherein E has the sequence of any one of SEQ ID NOs: 1-31.

869. A pharmaceutical composition comprising a conjugate of claim 462, or a pharmaceutically acceptable salt thereof, and a pharmaceutically acceptable excipient.

870. A method of:
(a) protecting against or treating a bacterial infection in a subject, said method comprising administering to said subject (1) a conjugate of claim 462 and (2) an antibacterial agent; or
(b) of preventing, stabilizing, or inhibiting the growth of bacteria, or killing bacteria, comprising contacting the bacteria or a site susceptible to bacterial growth with (1) a conjugate of claim 462 and (2) an antibacterial agent.


SEQUENCE LISTING (37 sequences)

SEQ ID NO: 1 - protein, 252 aa, Artificial Sequence
  Synthetic Construct
MYRMQLLSCIALSLALVTNSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 2 - protein, 232 aa, Mus musculus
PRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 3 - protein, 251 aa, Artificial Sequence
  Synthetic Construct
MYRMQLLSCIALSLALVTNSMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 231 aa, Artificial Sequence
  Synthetic Construct
MVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 258 aa, Artificial Sequence
  Synthetic Construct
MYRMQLLSCIALSLALVTNSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKHHHHHH

SEQ ID NO: 6 - protein, 238 aa, Artificial Sequence
  Synthetic Construct
PRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKHHHHHH

SEQ ID NO: 7 - protein, 257 aa, Artificial Sequence
  Synthetic Construct
MYRMQLLSCIALSLALVTNSMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 8 - protein, 237 aa, Artificial Sequence
  Synthetic Construct
MVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 9 - protein, 262 aa, Artificial Sequence
  Synthetic Construct
MYRMQLLSCIALSLALVTNSMVRSDKTHTCPPCPPCKCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 10 - protein, 242 aa, Artificial Sequence
  Synthetic Construct
MVRSDKTHTCPPCPPCKCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 11 - protein, 258 aa, Artificial Sequence
  Synthetic Construct
MYRMQLLSCIALSLALVTNSPRGPTIKPCPPCKCPAPNLLGGPSVFIFPPKIKDVLMISLSPIVTCVVVDVSEDDPDVQISWFVNNVEVHTAQTQTHREDYASTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKHHHHHH

SEQ ID NO: 12 - protein, 238 aa, Artificial Sequence
  Synthetic Construct
PRGPTIKPCPPCKCPAPNLLGGPSVFIFPPKIKDVLMISLSPIVTCVVVDVSEDDPDVQISWFVNNVEVHTAQTQTHREDYASTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKHHHHHH

SEQ ID NO: 13 - protein, 257 aa, Artificial Sequence
  Synthetic Construct
MYRMQLLSCIALSLALVTNSMVRSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 14 - protein, 237 aa, Artificial Sequence
  Synthetic Construct
MVRSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 15 - protein, 252 aa, Artificial Sequence
  Synthetic Construct
MKWVTFISLLFLFSSAYSISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 266 aa, Artificial Sequence
  Synthetic Construct
MKWVTFISLLFLFSSAYSISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQKLISEEDL

SEQ ID NO: 17 - protein, 248 aa, Artificial Sequence
  Synthetic Construct
ISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQKLISEEDL

SEQ ID NO: 18 - protein, 252 aa, Artificial Sequence
  Synthetic Construct
MKWVTFISLLFLFSSAYSISAMVRSDKTHTCPPCPAPELLGGPSVFLFPPKPSDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 234 aa, Homo sapiens
ISAMVRSDKTHTCPPCPAPELLGGPSVFLFPPKPSDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 266 aa, Artificial Sequence
  Synthetic Construct
MKWVTFISLLFLFSSAYSISAMVRSDKTHTCPPCPAPELLGGPSVFLFPPKPSDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQKLISEEDL

SEQ ID NO: 21 - protein, 248 aa, Artificial Sequence
  Synthetic Construct
ISAMVRSDKTHTCPPCPAPELLGGPSVFLFPPKPSDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQKLISEEDL

SEQ ID NO: 22 - protein, 266 aa, Artificial Sequence
  Synthetic Construct
MKWVTFISLLFLFSSAYSISAMVRSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQKLISEEDL

SEQ ID NO: 23 - protein, 248 aa, Artificial Sequence
  Synthetic Construct
ISAMVRSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQKLISEEDL

SEQ ID NO: 24 - protein, 266 aa, Artificial Sequence
  Synthetic Construct
MKWVTFISLLFLFSSAYSISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNAYTQKSLSLSPGGGGGSEQKLISEEDL

SEQ ID NO: 25 - protein, 248 aa, Artificial Sequence
  Synthetic Construct
ISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNAYTQKSLSLSPGGGGGSEQKLISEEDL

SEQ ID NO: 26 - protein, 266 aa, Artificial Sequence
  Synthetic Construct
MKWVTFISLLFLFSSAYSISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQFLISEEDL

SEQ ID NO: 27 - protein, 248 aa, Artificial Sequence
  Synthetic Construct
ISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQFLISEEDL

SEQ ID NO: 28 - protein, 266 aa, Artificial Sequence
  Synthetic Construct
MKWVTFISLLFLFSSAYSISAMVRSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQFLISEEDL

SEQ ID NO: 29 - protein, 248 aa, Artificial Sequence
  Synthetic Construct
ISAMVRSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQFLISEEDL

SEQ ID NO: 30 - protein, 259 aa, Artificial Sequence
  Synthetic Construct
MKWVTFISLLFLFSSAYSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQFLISEEDL

SEQ ID NO: 31 - protein, 245 aa, Artificial Sequence
  Synthetic Construct
MKWVTFISLLFLFSSAYSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 6 aa, Artificial Sequence
  Synthetic Construct
HHHHHH

SEQ ID NO: 33 - protein, 20 aa, Artificial Sequence
  Synthetic Construct
MYRMQLLSCIALSLALVTNS

SEQ ID NO: 34 - protein, 330 aa, Homo sapiens [sequence omitted]

SEQ ID NO: 35 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 36 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 37 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK